(19) United States
(12) Patent Application Publication
(10) Pub. No.: US 2024/0358842 A1
(43) Pub. Date: Oct. 31, 2024

(54) PROTEIN STABILIZING COMPOUNDS CONTAINING USP7 LIGANDS

(71) Applicant: Stablix, Inc., New York, NY (US)

(72) Inventors: Kevin Tyler SPROTT, New York, NY (US); Mark George SAULNIER, New York, NY (US); Moses MOUSTAKIM, New York, NY (US); Brian BOWMAN, New York, NY (US); Jorden KASS, New York, NY (US); Richard James STEEL, New York, NY (US)

(21) Appl. No.: 18/574,185

(22) PCT Filed: Jun. 24, 2022
(86) PCT No.: PCT/US2022/034998

§ 371 Date: Dec. 26, 2023

Related U.S. Application Data
(60) Provisional application No. 63/251,520, filed on Oct. 1, 2021.
(60) Provisional application No. 63/215,405, filed on Jun. 25, 2021.

(51) Int. Cl. A61K 47/55; A61K 47/54

(52) U.S. Cl. CPC A61K 47/55; A61K 47/545

(57) ABSTRACT

This invention provides protein stabilizing compounds, compositions, and methods of use thereof, that include a USP7 Targeting Ligand, a Protein Targeting Ligand, and optionally a Linker for the restoration of a Target Ubiquitinated Protein to treat a disorder mediated by deficiencies of the Target Protein.

### CROSS REFERENCE TO RELATED APPLICATIONS

This application claims the benefit of U.S. Provisional Application 63/215,405, filed on Jun. 25, 2021, and U.S. Provisional Application 63/251,520, filed on Oct. 1, 2021, the entirety of each of which is hereby incorporated by reference for all purposes.

### FIELD OF THE INVENTION

This invention provides bifunctional molecules that stabilize Target Ubiquitinated Proteins, compositions, and methods of use thereof. The bifunctional molecules include a USP7 Targeting Ligand, a Ubiquitinated Protein Targeting Ligand, and optionally a Linker that connects the two for the restoration of a Target Ubiquitinated Protein to treat a disorder mediated by deficiencies of the Target Protein.

### BACKGROUND OF THE INVENTION

The ubiquitination of proteins is a dynamic multifaceted post-translational modification that allows the body to mark proteins for degradation, sub-cellular localization, and translocation. Ubiquitin is a 76-amino acid protein that has several locations that can attach to other ubiquitins and other proteins. Ubiquitin commonly attaches to proteins at one of seven lysine residues or on the N-terminus. These reactive sites on ubiquitin can then be modified by other ubiquitin peptides or ubiquitin-like molecules (for example SUMO or NEDD8). The resulting three-dimensional polyubiquitin structure can be complex and can provide a multitude of signals. Swatek et. al., “Ubiquitin Modifications” Cell Research 2016 (26) 399. One of the common signals given by ubiquitin is that of proteasomal degradation. More than 700 E3 ubiquitin ligase proteins have been identified and these ligases can recognize ubiquitinated proteins and then orchestrate a complex cascade that results in protein degradation. Humphreys et. al., “The Role of E3 Ubiquitin Ligases in the Development and Progression of Glioblasoma” Cell Death & Differentiation 2021 (28) 522.

Difficult to treat diseases can occur when ubiquitination signals the degradation of proteins that the body needs. For example, in cystic fibrosis one or more mutations in the cystic fibrosis transmembrane conductance regulator (CFTR) gene causes CFTR to be less efficient in transporting ions in and out of the cellular membrane. Lee et. al., “Interference with Ubiquitination in CFTR Modifies Stability of Core Glycosylated and Cell Surface Pools” Mol. Cell Biol. 2014 (34) 2554. The body recognizes the mutant CFTR proteins as deficient and ubiquitinates them to signal degradation and thus makes the inability to transport ions in and out of the cell membrane even more pronounced. The result is a thickening of mucus, difficulty breathing, and eventual death.

The body has deubiquitinase proteins (DUBs) that partially or fully remove ubiquitin from proteins. There are over one hundred known DUBs. DUBS have been split into five families: the ubiquitin-specific proteases (USPs), the ovarian tumor proteases (OTUs), the ubiquitin C-terminal hydrolases (UCHs), the Josephin family, and the motif interacting with ubiquitin containing novel DUB family (MINDY). Mevissen et. al., “Mechanisms of Deubiquitinase Specificity and Regulation” Annu. Rev. Biochem. 2017 (86) 159. These DUBS have specificity for different functions and cleave different bonds in polyubiquitin.

The Colecraft lab has developed engineered DUB proteins “enDUBs” that have a highly selective nanobody portion connected to a DUB. Kanner et. al., “Targeted Deubiquitination Rescues Distinct Trafficking-Deficient Ion Channelopathies” Nature Methods 2020 (17) 1245. These molecules target a protein of interest, deubiquitinate it, and restore its function. Various enDUBs are disclosed in WO2019/090234, WO2020/198637, and WO2021/146390. Heterobifunctional molecules for targeted protein stabilization are described in WO2021/146386A1.

Locki Therapeutics Limited has described the use of small molecule compounds containing a protein targeting ligand, a linker, and a DUB targeting ligand for deubiquitinating the protein of interest in WO2020/169650.

The Nomura lab has described small molecule compounds containing a protein targeting ligand, a linker, and a DUB targeting ligand to deubiquitinate CFTR. Henning et. al., “Deubiquitinase-Targeting Chimeras for Targeted Protein Stabilization” Nature Chemical Biology 18, 412-421 (2022).

### SUMMARY OF THE INVENTION

Protein stabilizing and/or function restoring bifunctional compounds and their uses and manufacture are provided that stabilize a Target Ubiquitinated Protein by deubiquitinating it and in some embodiments restore at least a partial amount of the protein's function. The protein stabilizing and/or function restoring bifunctional compounds described herein include a ubiquitin specific protease 7 (USP7) Targeting Ligand, a Ubiquitinated Protein Targeting Ligand, and optionally a Linker that links the two. USP7 is a ˜128 kDa cysteine protease that can cleave at least 5 of the major polyubiquitin bonds (K6, K11, K33, K48, and K63-linked modifications).

USP7 is a key regulator of ubiquitination in protein degradation pathways. By interacting with USP7 and a Target Ubiquitinated Protein the protein stabilizing compounds described herein can restore a target protein's function and can thus be used to treat loss of function disorders

When USP7 removes ubiquitins from a protein, the proteasomal degradation of the protein may be prevented or minimized (i.e. the protein is stabilized), and thus the protein may resume its activity (i.e. the protein's function is restored). Alternatively, the deubiquitination may be insufficient to prevent degradation or restore function.

A selected compound described herein removes ubiquitin from the Target Ubiquitinated Protein in a manner that stabilizes the protein and in some embodiments restores the protein's function. For example, a compound of the present invention may increase a target protein's function by at least about 1%, 2.5%, 5%, 7.5%, 10%, 15%, 20%, 25%, 30%, 40%, 50%, 60%, 70%, 80%, 90%, 100%, or more, as compared to the target protein's level of function in the absence of the compound. In certain embodiments, the protein's function may be enhanced over the protein as existing in the cell prior to treatment with the compound described herein. When the target protein has a loss of function mutation a compound of the present invention may restore its function relative to the wild type protein or relative to the mutated form.

By both stabilizing and restoring the protein's function various disorders that are caused by a deficiency of a protein's activity can be treated. For example, disorders caused by loss of function protein mutations or haploid insufficiency can be treated by restoring the function of the downregulated wildtype protein of interest or a mutant thereof. Difficult to treat cancers can also be treated with a protein stabilizing compound of the present invention. For example, cancers that downregulate tumor suppressors can be treated by restoring the function of the tumor suppressor. A protein stabilizing compound described herein can also prompt an immunological response in the treatment of cancer and thus treat the cancer by activating the immune system.

In certain aspects of the invention a protein stabilizing compound is used in combination with a protein activating compound such as an agonist, potentiator, chaperone, or corrector to treat a disease mediated by the Target Ubiquitinated Protein. In other aspects the protein stabilizing compound prevents degradation of the Target Ubiquitinated Protein and that protein forms one or more complexes with downstream phenotypic effects. In certain embodiments the protein stabilizing compound stabilizes and restores the proteins activity.

In certain embodiments the USP7 Targeting Ligand used in the present invention is an inhibitor of USP7. Despite being an inhibitor of USP7 a USP7 Targeting Ligand promotes the deubiquitination, stabilization, and/or restoration of activity for the Targeted Protein when used within a compound described herein. In certain embodiments the USP7 Targeting Ligand binds an allosteric site with inhibitor activity. In other embodiments the USP7 Targeting Ligand binds an active site.

In certain embodiments the USP7 Targeting Ligand used in the present invention is not an inhibitor of USP7. For example, in certain embodiments the USP7 Targeting Ligand is an agonist, activator, potentiator, or ligand without appreciable binding activity.

In certain aspects a protein stabilizing compound of Formula I is provided:

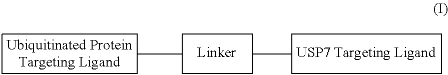

or a pharmaceutically acceptable salt thereof;
wherein:
the Ubiquitinated Protein Targeting Ligand is a ligand that binds a Target Ubiquitinated Protein; in certain embodiments the Protein's biological function can be fully or partially restored by deubiquitination as described herein;
the Linker is a bond or a bivalent moiety that links the Protein Targeting Ligand and the USP7 Targeting; and
the USP7 Targeting Ligand is a USP7 Targeting Ligand described herein for example a compound in FIG. 1 that binds USP7.

In certain embodiments the compound of the present invention is of Formula:

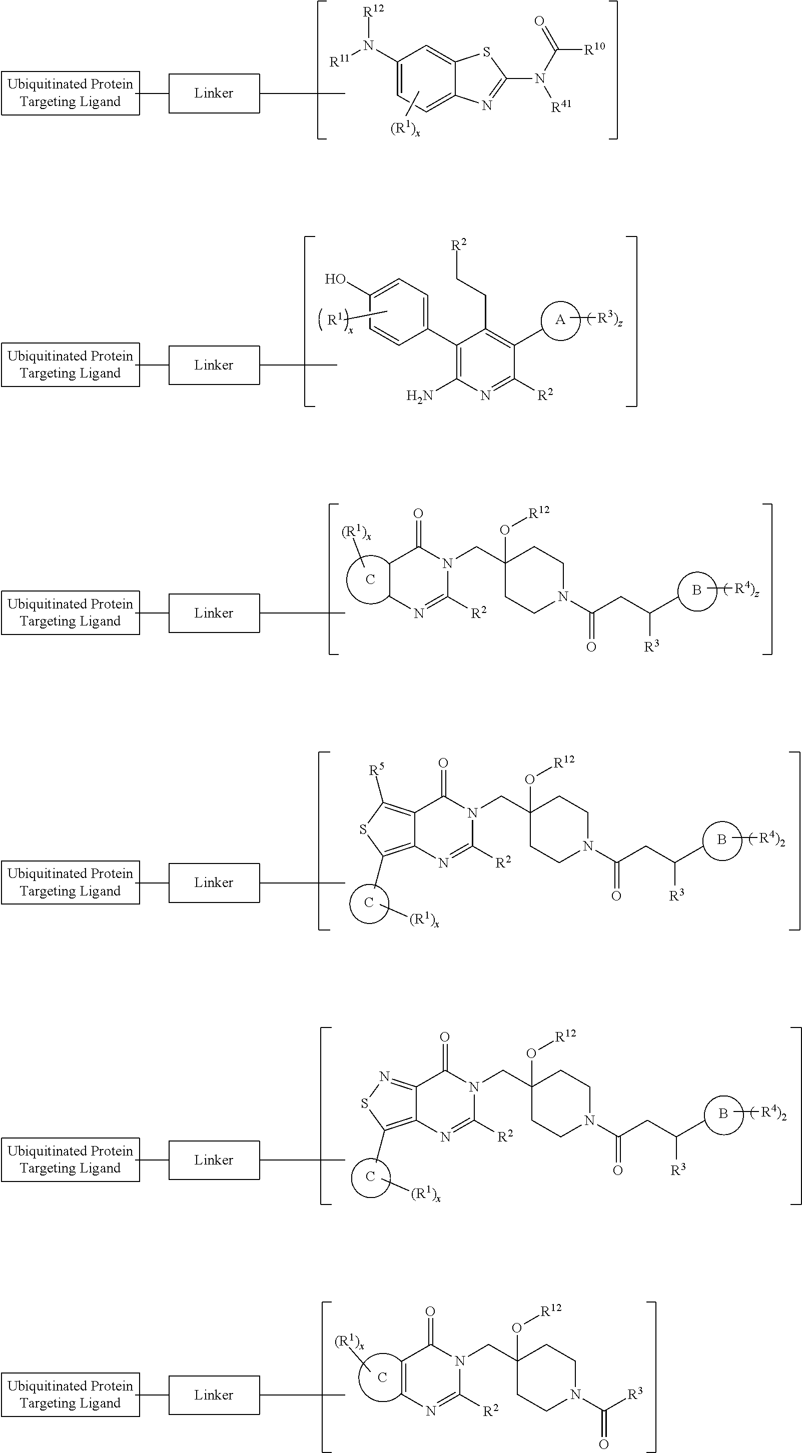

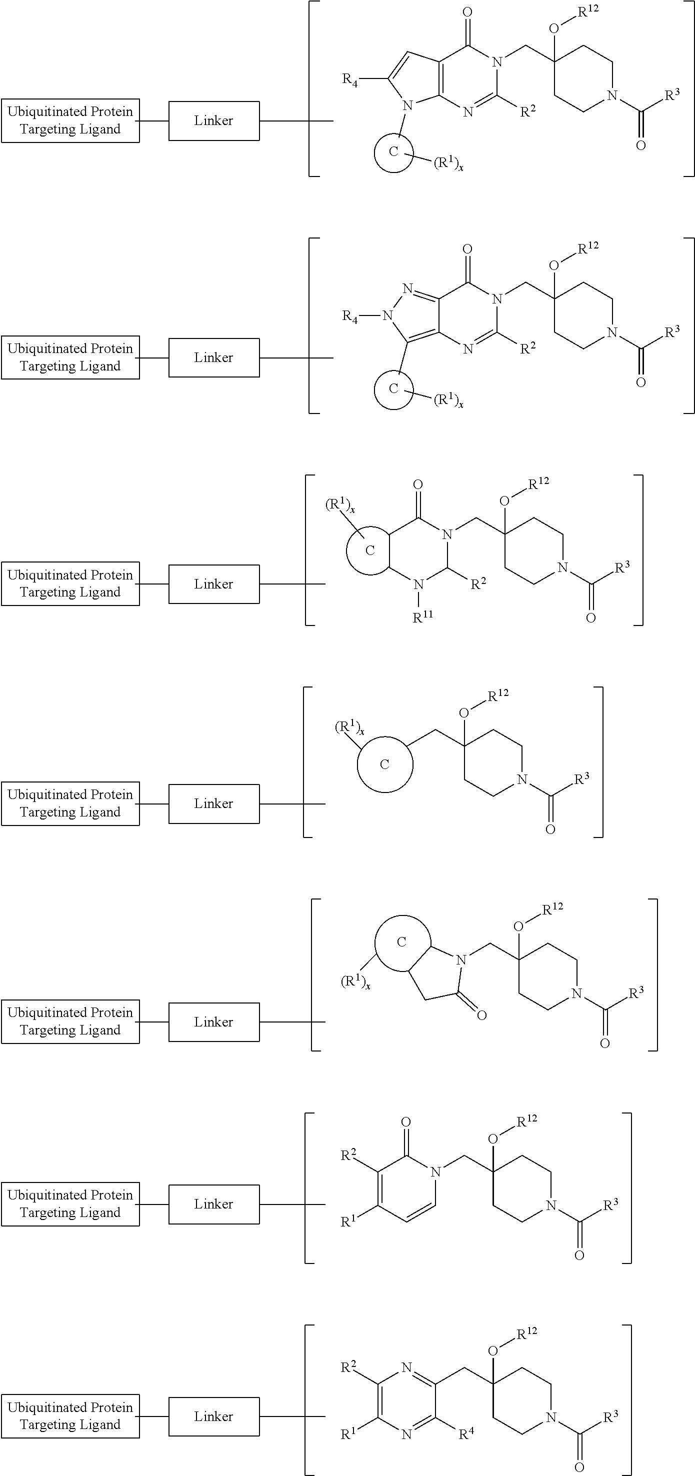

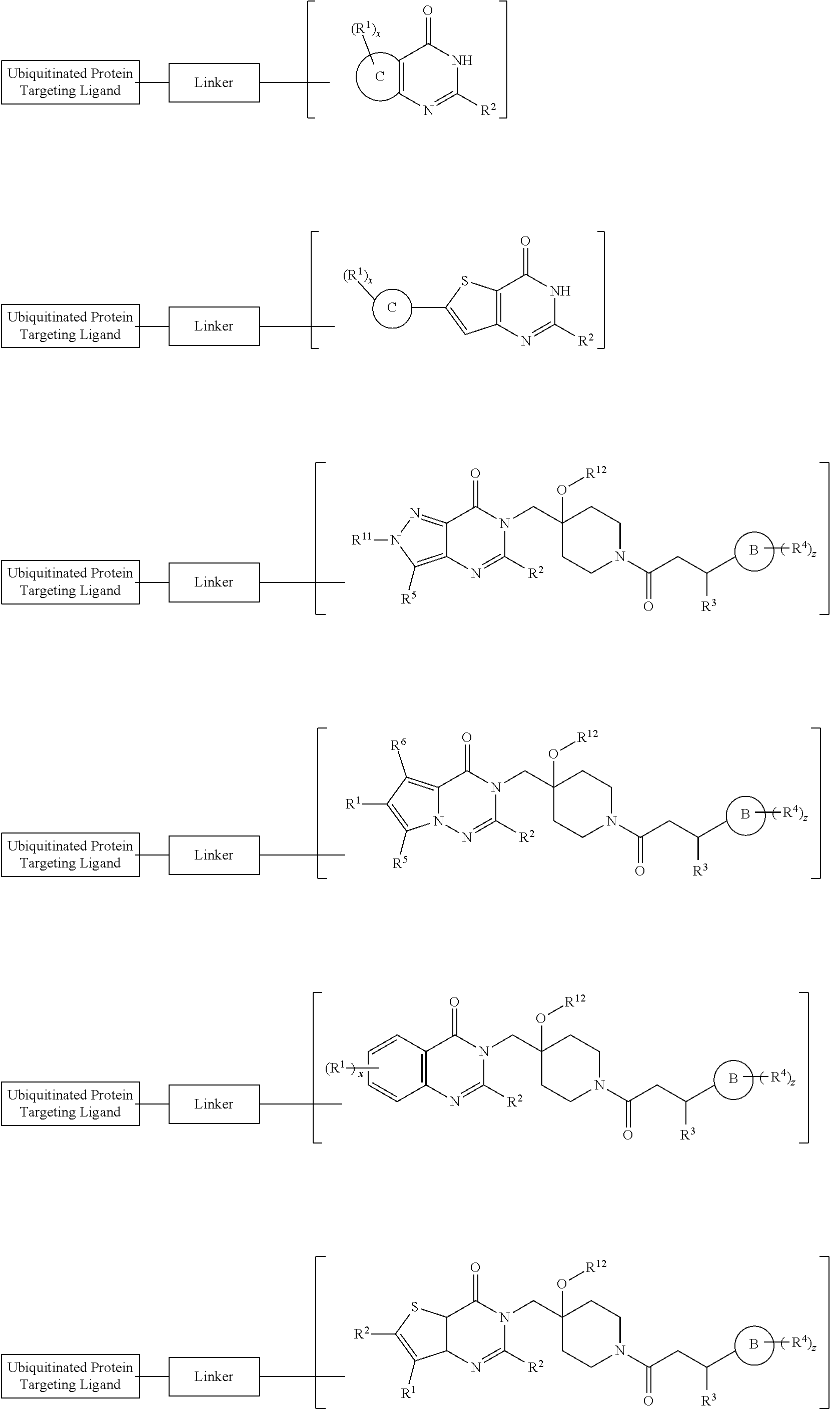

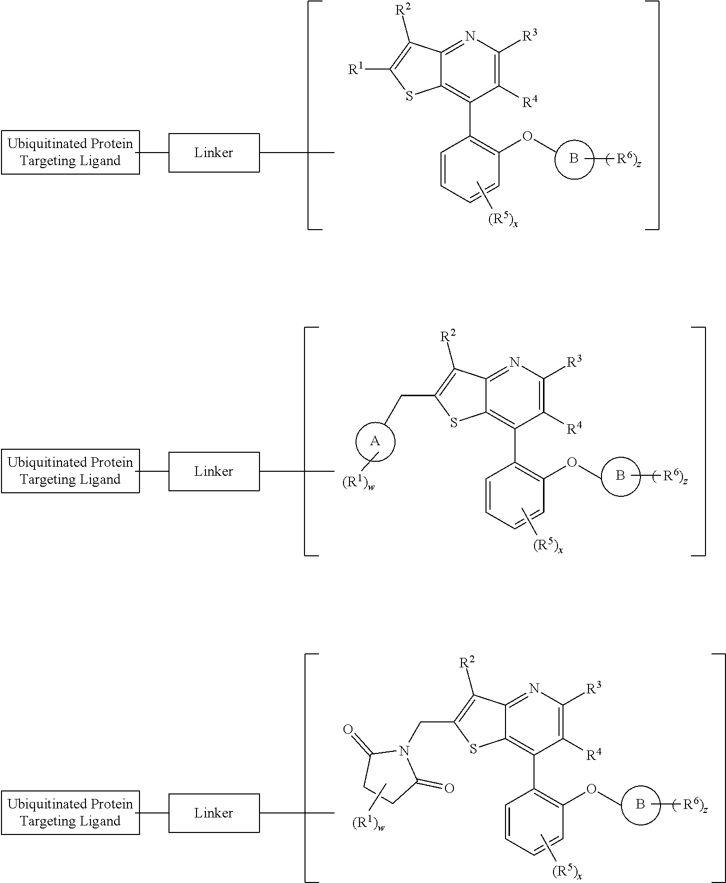

or a pharmaceutically acceptable salt thereof;
wherein:

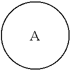

is an aryl, heteroaryl, heterocycle, or cycloalkyl group;

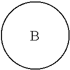

is an aryl, heteroaryl, heterocycle, or cycloalkyl group;

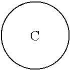

is an aryl, heteroaryl, heterocycle, or cycloalkyl group;

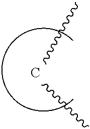

is a fused heterocycle, aryl, heteroaryl, cycloalkyl, or cycloalkenyl group;
x is 0, 1, 2, 3, or 4 as allowed by valence;
z is 0, 1, 2, 3, or 4 as allowed by valence;
w is 0, 1, 2, 3, or 4 as allowed by valence;
R¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹;
R² is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²²;
R³ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²³;
R⁴ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁴;
R⁵ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁵;
R⁶ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁶;
R¹⁰ is independently selected at each instance from hydrogen, alkyl, haloalkyl, alkenyl, alkynyl, —OR¹¹, —NR¹¹R¹², —SR¹¹, aryl, heterocycle, and heteroaryl; each of which alkyl, haloalkyl, alkenyl, alkynyl, heterocycle, aryl, and heteroaryl is optionally substituted as allowed by valence with 1, 2, 3, or 4 substituents selected from R³⁰;
R¹¹ and R¹² are independently selected at each instance from hydrogen, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
R²¹, R²², R²³, R²⁴, R²⁵, and R²⁶ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R³⁰ and R³¹ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R⁴⁰ is independently selected at each instance from hydrogen, alkyl, haloalkyl, alkenyl, alkynyl, aryl, heterocycle, heteroaryl, amino, hydroxyl, alkoxy, —NHalkyl, and —N(alkyl)₂, each of which except hydrogen is optionally substituted as allowed by valence with 1, 2, 3, or 4 substituents selected from R⁴³;
R⁴¹ and R⁴² are independently selected at each instance from hydrogen, alkyl, haloalkyl, alkenyl, alkynyl, aryl, heterocycle, and heteroaryl; each of which except hydrogen is optionally substituted as allowed by valence with 1, 2, 3, or 4 substituents selected from R⁴³; and
R⁴³ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl.

In certain embodiments the Linker-Ubiquitinated Protein Targeting Ligand replaces a R¹, R², R³, R⁴, R⁵, R⁶, R¹⁰, R¹¹, or R¹². In another embodiment Linker-Ubiquitinated Protein Targeting Ligand is covalently attached to a R¹, R², R³, R⁴, R⁵, R⁶, R¹⁰, R¹¹, or R² as allowed by valence. In another embodiment, the Linker is covalently bound in a position other than R¹, R², R³, R⁴, R⁵, R⁶, R¹⁰, R¹¹, or R¹².

In certain embodiments Linker is of Formula:

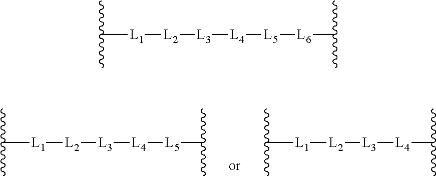

wherein
L₁, L₂, L₃, L₄, L₅, and L₆ are independently selected from the group consisting of a bond, alkyl, alkene, alkyne, haloalkyl, alkoxy, aryl, heterocycle, heteroaryl, bicycle, —C(O)—, —C(O)O—, —OC(O)—, —SO₂—, —S(O)—, —C(S)—, —C(O)NR¹¹—, —NR¹¹C(O)—, —O—, —S—, —NR¹¹—, —P(O)(OR¹¹)O—, —P(O)(OR¹¹)—, polyethylene glycol, lactic acid, and glycolic acid, each of which except bond is optionally substituted with 1, 2, 3, or 4 substituents independently selected from R⁴⁴; wherein L₁, L₂, L₃, L₄, L₅, and L₆ are selected such that there are no more than two of the same moieties connected together (e.g, L₁, L₂, and L₃ cannot all three be —C(O)—) and O and N atoms are not directly linked together except within aromatic rings (e.g. L₁ and L₂ cannot both be —O— or NR¹¹);
R⁴⁴ is independently selected at each instance from hydrogen, alkyl, haloalkyl, alkenyl, alkynyl, aryl, heterocycle, heteroaryl, amino, hydroxyl, alkoxy, —NR¹¹R¹², halogen, cyano, nitro, —OC(O)R⁴⁰, —NR¹¹C(O)R⁴⁰, —C(O)R⁴⁰, —OP(O)(R⁴⁰)₂, —P(O)(R⁴⁰)₂, —NR¹¹P(O)(R⁴⁰)₂, —SR¹¹, —OR¹¹, —S(O)R⁴⁰, —S(O)₂R⁴⁰, and —N(alkyl)C(O)R⁴⁰, each of which except hydrogen is optionally substituted as allowed by valence with 1, 2, 3, or 4 substituents selected from R⁴⁵; and
R⁴⁵ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl.

In certain aspects a protein stabilizing compound of Formula II is provided:

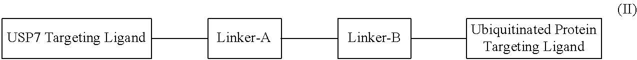

or a pharmaceutically acceptable salt thereof;
wherein
Linker-A is a bivalent moiety that links Linker-B and the USP7 Targeting; and
Linker-B is a bivalent moiety that links the Ubiquitinated Protein Targeting Ligand and Linker-A.

In certain embodiments Linker-A is of Formula:

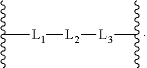

In certain embodiments Linker-B is of Formula:

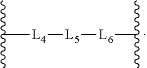

In certain embodiments, the Ubiquitinated Protein Targeting Ligand is a pharmaceutical organic ligand (e.g. not an inorganic substance, that binds to the Target Ubiquitinated Protein adequately to facilitate deubiquitination. In certain embodiments of the invention, the Ubiquitinated Protein Targeting Ligand is a peptide or oligonucleotide that binds to the Target Ubiquitinated Protein adequately to facilitate deubiquitination. In certain embodiments the Ubiquitinated Protein Targeting Ligand is a pharmaceutically active compound or a fragment thereof that binds to the Target Ubiquitinated Protein (for example an approved drug or a compound in development with known binding affinity for the Target Ubiquitinated Protein in either the ubiquitinated or nonubiquitinated form). A plethora of illustrative nonlimiting examples or Ubiquitinated Protein Targeting Ligands for use in the present invention are provided in the Detailed Description and Figures. Additional Ubiquitinated Protein Targeting Ligand are known in the art.

The protein stabilizing compounds described herein stabilize and restore function to a Target Protein by deubiquitinating the corresponding Target Ubiquitinated Protein. For example, when the Ubiquitinated Protein Targeting Ligand is an inhibitor of the Target Ubiquitinated Protein then the protein stabilizing compound will deubiquitinate the Target Ubiquitinated Protein and at least partially restore its function, however, the Target Ubiquitinated Protein's activity will not be increased beyond the activity of the non-ubiquitinated version of the protein. In other embodiments a protein stabilizing compound described herein stabilizes, restores, and activates the Target Ubiquitinated Protein. For example, when the Ubiquitinated Protein Targeting Ligand is an agonist or activator of the Target Ubiquitinated Protein then the protein stabilizing compound will deubiquitinate the Target Ubiquitinated Protein, restore its function, and increase its activity.

By restoring function to proteins which have beneficial activity the compounds described herein can be used to treat a variety of difficult to treat disorders. Non-limiting examples of Target Ubiquitinated Proteins include RIPK1, BRD7, c-Myc, rhodopsin, p53, PAH, CFTR, MSH2, PDCD4, p27-kip1, ABCA4, and ABCB11-4 or a wild type, mutant forms, splice variant, or altered sequence thereof. Additional examples of Target Ubiquitinated Proteins include KEAP1, PKLR, KCNQ1, TK2, STING1, IRAK4, PTEN, SERPINA1, P21, BAX, and RIPK2 or a wild type, mutant forms, splice variant, or altered sequence thereof. In certain embodiments, a method of treating a disorder mediated by a Target Ubiquitinated Protein is provided comprising administering an effective amount of a protein stabilizing compound described herein, or a pharmaceutically acceptable salt thereof, to a patient in need thereof, for example a human, optionally in a pharmaceutically acceptable carrier. For example, in certain embodiments, a protein stabilizing compound of Formula I or Formula II, is administered to a human to treat a cancer or tumor where the protein stabilizing compound has a Ubiquitinated Protein Targeting Ligand that binds the Target Ubiquitinated Protein, and the tumor or cancer is mediated by the Target Ubiquitinated Protein.

In certain embodiments the Target Ubiquitinated Protein is ChAT (for example P17A/P19A mutant ChAT), CYLD (for example missense mutant CYLD), NEMO, AIP (for example missense AIP or nonsense mutant AIP), or Eya1 (for example S454P, L472R, or L550P Eya1).

Non-limiting examples of disorders that can be treated by a protein stabilizing compound of the present invention include cystic fibrosis (for example wherein the compound stabilizes CFTR or a mutant thereof), phenylketonuria (for example wherein the compound stabilizes PAH or a mutant thereof), progressive familial intrahepatic cholestasis (for example wherein the compound stabilizes ABCB11/4 or a mutant thereof), Stargardt Disease (for example wherein the compound stabilizes ABCA4 or a mutant thereof), retinitis pigmentosa (for example wherein the compound stabilizes rhodopsin or a mutant thereof), or a cancer (for example wherein the compound stabilizes p53, cMyc, P27^{Kip1}, PDCD4, MSH2, or RIPK1 or a mutant thereof).

Additional non-limiting examples of disorders that can be treated by a protein stabilizing compound of the present invention include congenital myasthenic syndrome (for example wherein the compound stabilizes ChAT or a mutant thereof), Brooke-Spiegler syndrome (for example wherein the protein stabilizes CYLD or NEMO or a mutant thereof), pituitary adenoma (for example wherein the compound stabilizes AIP or a mutant thereof), or BOR syndrome (for example wherein the protein stabilizes Eya1 or a mutant thereof).

A protein stabilizing compound of the present invention can be administered in any manner that allows the compound to stabilize the Target Ubiquitinated Protein's and/or restore its function. As such, examples of methods to deliver the protein stabilizing compound of the present invention include, but are not limited to, systemic, parenteral, topical, oral, intravenous, buccal, sublingual, subcutaneous, or transnasal administration.

In certain embodiments, the protein stabilizing compound of the present invention has at least one desired isotopic substitution of an atom, at an amount above the natural abundance of the isotope, i.e., enriched.

In certain embodiments, the protein stabilizing compound of the present invention includes a deuterium or multiple deuterium atoms.

Another aspect of the present invention provides a protein stabilizing compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing a disease in which the Target Ubiquitinated Protein plays a role.

In certain embodiments a method of stabilizing and restoring a protein's function is provided. The skilled artisan will recognize how to assess whether or not a protein's function has been restored in vivo or in vitro depending on context. For example, when the Target Ubiquitinated Protein is an ion channel, such as CFTR, surface representation assays or ion current assays can be used to assay protein function restoration in vitro. Additionally, a reduction of symptoms associated with a disease mediated by the Target Ubiquitinated Protein will show in vivo efficacy. For example, when the Target Ubiquitinated Protein is CFTR amelioration of cystic fibrosis symptoms will result from protein function restoration in vivo. When the Target Ubiquitinated Protein is an oncological target, such as p53, cell death assays or cell cycle assays can be used to demonstrate the restoration of function. When the Target Ubiquitinated Protein is an enzyme then its enzymatic activity can be assayed to demonstrate the restoration of function.

Other features and advantages of the present application will be apparent from the following detailed description.

The present invention thus includes at least the following features:
(a) A protein stabilizing compound of Formula I or Formula II as described herein, or a pharmaceutically acceptable salt or isotopic derivative (including a deuterated derivative) thereof;
(b) A method for treating a disorder mediated by a Target Ubiquitinated Protein, comprising administering an effective amount of a protein stabilizing compound of Formula I or Formula II, or pharmaceutically acceptable salt thereof, as described herein, to a patient in need thereof wherein the protein stabilizing compound contains a Ubiquitinated Protein Targeting Ligand that binds the Target Ubiquitinated Protein;
(c) A protein stabilizing compound of Formula I or Formula II, or a pharmaceutically acceptable salt thereof for use in the treatment of a disorder that is mediated by a Target Ubiquitinated Protein, wherein the protein stabilizing compound contains a Ubiquitinated Protein Targeting Ligand that binds the Target Ubiquitinated Protein;
(d) Use of a protein stabilizing compound of Formula I or Formula II, or a pharmaceutically acceptable salt thereof, in an effective amount in the treatment of a patient in need thereof, typically a human, with disorder mediated by a Target Ubiquitinated Protein, wherein the protein stabilizing compound contains a Ubiquitinated Protein Targeting Ligand that binds the Target Ubiquitinated Protein;
(e) Use of a protein stabilizing compound of Formula I or Formula II, or a pharmaceutically acceptable salt thereof in the manufacture of a medicament for the treatment of a disorder mediated by a Ubiquitinated Protein Targeting Ligand that binds the Target Ubiquitinated Protein;
(f) A pharmaceutical composition comprising a protein stabilizing compound of Formula I or Formula II, or a pharmaceutically acceptable salt thereof; and a pharmaceutically acceptable carrier or diluent;
(g) A protein stabilizing compound of Formula I or Formula II, as described herein as a mixture of enantiomers or diastereomers (as relevant), including as a racemate;
(h) A protein stabilizing compound of Formula I or Formula II, as described herein in enantiomerically or diastereomerically (as relevant) enriched form, including an isolated enantiomer or diastereomer (i.e., greater than 85, 90, 95, 97, or 99% pure); and
(i) A process for the preparation of therapeutic products that contain an effective amount of a protein stabilizing compound of Formula I or Formula II, or a pharmaceutically acceptable salt thereof, as described herein.

### BRIEF DESCRIPTION OF THE FIGURES

As used in the figures:
y is 0, 1, 2, or 3;
R⁹⁹ is the attachment point to Linker-Ubiquitinated Protein Targeting Ligand;
R¹⁰⁰ is the attachment point to Linker-USP7 Targeting Ligand;
R²⁰⁰ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹¹, wherein each alkyl, haloalkyl, alkenyl, alkynyl, heterocycle, aryl, and heteroaryl is optionally substituted as allowed by valence with 1, 2, 3, or 4 substituents selected from R²¹.

As used herein, where a cyclic group within a drawn molecule has a number in the middle of the cycle these numbers are used to denote cycles to which the Linker may be attached as allowed by valence.

In certain embodiments the Linker is attached to the cycle marked with a 1.

In certain embodiments the Linker is attached to the cycle marked with a 2.

In certain embodiments the Linker is attached to the cycle marked with a 3.

In certain embodiments the Linker is attached to the cycle marked with a 4.

In certain embodiments the Linker is attached to the cycle marked with a 5.

In certain embodiments the Linker is attached to the cycle marked with a 6.

For example

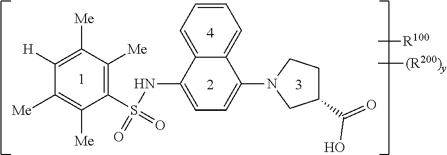

when attached to the Linker in the cycle marked with a 1 includes the following non-limiting exemplary structure:

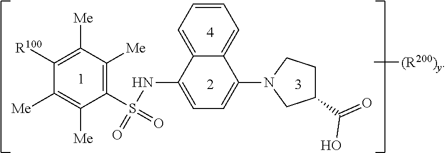

Where a substituent is already on the cycle marked 1, 2, 3, 4, 5, or 6, the linker may be on or replace that substituent as allowed by valence. For example

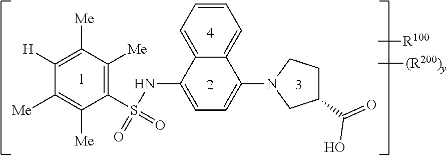

when attached to the Linker in the cycle marked with a 1 also includes the following non-limiting exemplary structures:

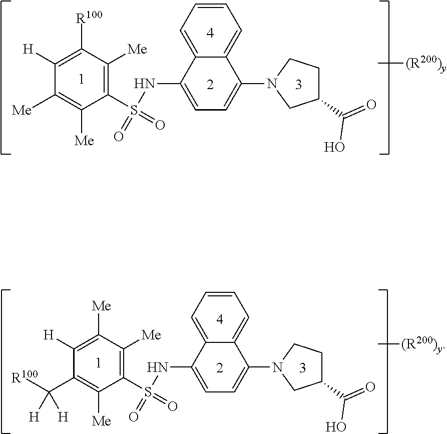

FIG. 1A, FIG. 1B, FIG. 1C, FIG. 1D, FIG. 1E, FIG. 1F, FIG. 1G, FIG. 1H, and FIG. 1I present non-limiting examples of ligands that bind to Ubiquitin Specific Peptidase 7 (USP7), including the compounds 8JM, 8JP, R3Y, R4J, R44, R41, R4D, EZF, 8WN, 8WK, CQ5, 8RN, 8QQ, 9QA, 9HS, 9QD, AJJ, XL203C, I-28, and I-117. For additional non-limiting examples and related ligands, see ligands identified by Kategaya et al., “USP7 small-molecule inhibitors interfere with ubiquitin binding”, Nature, 2017, 550: 534-538; Leger et. al., “Discovery of Potent, Selective, and Orally Bioavailable Inhibitors of USP7 with In Vivo Antitumor Activity”, J Med Chem., 2020, 63: 5398-5420; Li et al., “N-benzylpiperidinol derivatives as novel USP7 inhibitors: Structure-activity relationships and X-ray crystallographic studies”, Eur J Med Chem., 2020, 199: 112279-112279; Turnbull et al., “Molecular basis of USP7 inhibition by selective small-molecule inhibitors”, Nature, 2017, 550: 481-486; O'Dowd et al., “Identification and Structure-Guided Development of Pyrimidinone Based USP7 Inhibitors”, ACS Med Chem Lett., 2018, 9: 238-243; Gavory et al., “Discovery and characterization of highly potent and selective allosteric USP7 inhibitors”, Nat Chem Biol., 2018, 14: 118-125; Lamberto et al., “Structure-Guided Development of a Potent and Selective Non-covalent Active-Site Inhibitor of USP7”, Cell Chem Biol., 2017, 24: 1490-1500.e11; Di Lello et al., “Discovery of Small-Molecule Inhibitors of Ubiquitin Specific Protease 7 (USP7) Using Integrated NMR and in Silico Techniques”, J Med Chem., 2017, 60: 10056-10070; Vamisetti et al., “Halogen Substituents in the Isoquinoline Scaffold Switches the Selectivity of Inhibition between USP2 and USP7”, ChemBioChem, 2019, 20: 282; Li et al. “Design, synthesis, biological evaluation and structure-activity relationship study of quinazolin-4(3H)-one derivatives as novel USP7 inhibitors”, Eur J Med Chem., 2021, 216: 113291; Varca et al., “Identification and Validation of Selective Deubiquitinase Inhibitors” Cell Chem. Bio. 2021; WO2019067503; WO2013030218; CN112047933; WO2016109480; WO2017212010; WO2017212012; US20200095260A1; US20190142834; WO2016150800; CN111808105; WO2018073602; and WO2018183587.

FIG. 2A, FIG. 2B, FIG. 2C, and FIG. 2D present non-limiting examples of ligands that bind to Cystic fibrosis transmembrane conductance regulator (CFTR), including the compounds LIP, CLR, AJP, VX7, POV, FSC, AP5, 4HY, A99, 64N, 64L, and 64O. For additional non-limiting examples and related ligands, see ligands identified by Liu, F., et al., “Structural identification of a hotspot on CFTR for potentiation”, Science, 2019, 364: 1184-1188; Stevers, L. M., et al., “Characterization and small-molecule stabilization of the multisite tandem binding between 14-3-3 and the R domain of CFTR”, Proc Natl Acad Sci USA, 2016,113: E1152-E1161; Lammens, A., Hopfner, K. P., “Structural Basis for Adenylate Kinase Activity in ABC ATPases”, J Mol Biol., 2010, 401: 265-273; Bahl, C. D., et al., “ ”, Angew Chem Int Ed Engl., 2015, 54: 9881-9885; Voellmecke, C., et al., “Conformational Changes in the Catalytic Domain of the Cpx-ATPase Copb-B Upon Nucleotide Binding”, to be published; Kitamura, S., et al., “Rational Design of Potent and Selective Inhibitors of an Epoxide Hydrolase Virulence Factor from Pseudomonas aeruginosa”, J Med Chem., 2016, 59: 4790-4799; Ridley K, et al., “Elexacaftor-Tezacaftor-Ivacaftor: The First Tripie-Combination Cystic Fibrosis Transmembrane Conductance Regulator Modulating” Therapy, J Pediatr Pharmacol Ther. 2020; 25(3):192-197; Ghelani et at, “Emerging Cystic Fibrosis Transmembrane Conductance Regulator Modulators as New Drugs for Cystic Fibrosis: A Portrait of in Vitro Pharmacology and Clinical Translation” ACS Pharmacol. Transl. Sci. 2020, 3, 1, 4-10; Fiedorczuk K, et al., “Mechanism of CFTR Correction by Type I Folding Correctors, bioRxiv prepring 2021, doi.org/10.1101/2021.06.18.449063; Grand et al., “Discovery of Icenticaftor (GBW251), a Cystic Fibrosis Transmembrane Conductance Regulator Potentiator with Clinical Efficacy in Cystic Fibrosis and Chronic Obstructive Pulmonary Disease” J. Med. Chem 2021, 64, 11, 7241-7260; Plas et al.; “Discovery of GLPG2451, a Novel Once Daily Potentiator for the Treatment of Cystic Fibrosis” J. Med. Chem. 2021, 64, 1, 343-353; Hadida et al., “Discovery of N-(2,4-Di-tert-butyl-5-hydroxyphenyl)-4-oxo-1,4-dihydroquinoline-3-carboxamide (VX-770, Ivacaftor), a Potent and Orally Bioavailable CFTR Potentiator” J. Med. Chem. 2014, 57, 23, 9776-9795; Hughes “Patent Review of Synthetic Routes and Crystalline Forms of the CFTR-Modulator Drugs Ivacaftor, Lumacaftor, Tezacaftor, and Elexacaftor” Org. Process Res. Dev. 2019, 23, 11, 2302-2322. Plas et al., “Discovery of N-(3-Carbamoyl-5,5,7,7-tetramethyl-5,7-dihydro-4H-thieno[2,3-c]pyran-2-yl)-IH-pyrazole-5-carboxamide(GLPG1837), a Novel Potentiator Which Can Open Class III Mutant Cystic Fibrosis Transmembrane Conductance Regulator (CFTR) Channels to a High Extent” J. Med. Chem. 2018, 61, 4, 1425-1435; Wang et al., “Discovery of 4-[(2R,4R)-4-({[1-(2,2-Difluoro-1,3-benzodioxol-5-yl)cyclopropyl]carbonyl}amino)-7-(difluoromethoxy)-3,4-dihydro-2H-chromen-2-yl]benzoic Acid (ABBV/GLPG-2222), a Potent Cystic Fibrosis Transmembrane Conductance Regulator (CFTR) Corrector for the Treatment of Cystic Fibrosis” J. Med. Chem. 2018, 61, 4, 1436-1449.

FIG. 3A, FIG. 3B, and FIG. 3C present non-limiting examples of ligands that bind to Phenylalanine Hydroxylase (PAH) including the compounds PHE, HBI, 3QI, H4B, TIH, H2B, XDE, LNR, LDP, DAH, and PIN. For additional non-limiting examples and related ligands, see ligands identified by Ronau et al., “An additional substrate binding site in a bacterial phenylalanine hydroxylase”, Eur Biophys J., 2013, 42: 691-708; Erlandsen et al., “Structural comparison of bacterial and human iron-dependent phenylalanine hydroxylases: similar fold, different stability and reaction rates”, J Mol Biol., 2002, 320: 645-661; Torreblanca et al., “Structural and Mechanistic Basis of the Interaction between a Pharmacological Chaperone and Human Phenylalanine Hydroxylase”, Chembiochem., 2012, 13: 1266; Anderson et al., “Crystal Structure of the Ternary Complex of the Catalytic Domain of Human Phenylalanine Hydroxylase with Tetrahydrobiopterin and 3-(2-thienyl)-L-alanine, and its Implications for the Mechanism of Catalysis and Substrate Activation”, J Mol Biol., 2002, 320: 1095-1108; Erlandsen et al., “Correction of kinetic and stability defects by tetrahydrobiopterin in phenylketonuria patients with certain phenylalanine hydroxylase mutations”, Proc Natl Acad Sci USA, 2004, 101: 16903-16908; Erlandsen et al., “Crystallographic analysis of the human phenylalanine hydroxylase catalytic domain with bound catechol inhibitors at 2.0 A resolution”, Biochemistry, 1998m 37: 15638-15646; Zhuang et al., “Phenylalanine hydroxylase from dictyostelium—BH2 complex”, to be published; Perchik et al., “The Effects of Ligand Deprotonation on the Binding Selectivity of the Phenylalanine Hydroxylase Active Site” Computation and Theoretical Chemistry, 2019, 1153, 19-24.

FIG. 4A, FIG. 4B, and FIG. 4C present non-limiting examples of ligands that bind to Tumor protein P53 (p53). For additional non-limiting examples and related ligands, see ligands identified by Baud et al., “Aminobenzothiazole derivatives stabilize the thermolabile p53 cancer mutant Y220C and show anticancer activity in p53-Y220C cell lines”, Eur J Med Chem., 2018, 152: 101-114; Allen et al., “Discovery and optimization of chromenotriazolopyrimidines as potent inhibitors of the mouse double minute 2-tumor protein 53 protein-protein interaction”, J Med Chem., 2009, 52: 7044-7053; Bauer et al., “A structure-guided molecular chaperone approach for restoring the transcriptional activity of the p53 cancer mutant Y220C”, Future Med Chem., 2019, 11: 2491-2504; Boeckler et al., “Targeted Rescue of a Destabilized Mutant of P53 by an in Silico Screened Drug”, Proc Natl Acad Sci USA, 2008, 105: 10360; Liu et al., “Small molecule induced reactivation of mutant p53 in cancer cells”, Nucleic Acids Res., 2013, 41: 6034-6044; Wilcken et al., “Halogen-Enriched Fragment Libraries as Leads for Drug Rescue of Mutant P53”, J Am Chem Soc., 2012, 134: 6810; Bauer et al., “Harnessing Fluorine-Sulfur Contacts and Multipolar Interactions for the Design of P53 Mutant Y220C Rescue Drugs”, ACS Chem Biol., 2016, 11: 2265; Joerger et al., “Exploiting Transient Protein States for the Design of Small-Molecule Stabilizers of Mutant P53”, Structure, 2015, 23: 2246; Basse et al., “Toward the Rational Design of p53-Stabilizing Drugs: Probing the Surface of the Oncogenic Y220C Mutant”, Chemistry and Biology, 2010, 29, 46-56.

FIG. 5A and FIG. 5B presents non-limiting examples of ligands that bind to Rhodopsin including the compounds DOK, DNZ, DO5, DL2, DLB, DLH, DN5, and 7AB. For additional non-limiting examples and related ligands, see ligands identified by Murakami et al., “Crystallographic Analysis of the Primary Photochemical Reaction of Squid Rhodopsin”, J Mol Biol., 2011, 413: 615-627; Okada et al., “Functional role of internal water molecules in rhodopsin revealed by X-ray crystallography”, Proc Natl Acad Sci USA, 2002, 99: 5982-5987; Mattle et al., “Ligand channel in pharmacologically stabilized rhodopsin”, Proc Natl Acad Sci USA., 2018, 115: 3640-3645; Gulati et al., “Photocyclic behavior of rhodopsin induced by an atypical isomerization mechanism”, Proc Natl Acad Sci USA, 2017, 114: E2608-E2615, Zhou et al. “Structure and Activation of Rhodopsin”, Acta Pharmacol Sin. 2020, 33, 291-299.

FIG. 6A and FIG. 6B present non-limiting examples of ligands that bind to c-Myc including the compounds QUL, 9WP, B06, QUE, Q8P, Q8D, Q8G, Q8S, Q8M, and QF1. For additional non-limiting examples and related ligands, see ligands identified by Dai et al., “Solution Structure of a 2:1 Quindoline-c-MYC G-Quadruplex: Insights into G-Quadruplex-Interactive Small Molecule Drug Design”, J Am Chem Soc., 2011, 133: 17673-17680; Calabrese et al., “Chemical and structural studies provide a mechanistic basis for recognition of the MYC G-quadruplex”, Nat Commun., 2018, 9: 4229-4229; Liu et al., “Structures of 1:1 and 2:1 complexes of BMVC and MYC promoter G-quadruplex reveal a mechanism of ligand conformation adjustment for G4-recognition”, Nucleic Acids Res., 2019, 47: 11931-11942; Kumar et al., “Solution structure for quercetin complexed with c-myc G-quadruplex DNA”, to be published; Chacon Simon et al., “Discovery of WD Repeat-Containing Protein 5 (WDR5)-MYC Inhibitors Using Fragment-Based Methods and Structure-Based Design”, J Med Chem., 2020, 63: 4315-4333; Whitefield et al., “Strategies to Inhibit Myc and Their Clinical Applicability” Front Cell Dev. Biol., 2017, 5, 10.

FIG. 7A, FIG. 7B, FIG. 7C, FIG. 7D, and FIG. 7E present non-limiting examples of ligands that bind to Receptor-interacting protein kinase 1 (RIPK1 or RIP1 kinase) including the compounds L4Y, L8D, NAG, UDP, EJP, EJY, LN4, QOK, RCM, 1HW, 1HX, Q1A, 65U, M5J, JSW, 7MJ, K8K, and G4W. For additional non-limiting examples and related ligands, see ligands identified by Hamilton et al., “Potent and selective inhibitors of receptor-interacting protein kinase 1 that lack an aromatic back pocket group”, Bioorg Med Chem Lett., 2019, 29: 1497-1501; Patel et al., “RIP1 inhibition blocks inflammatory diseases but not tumor growth or metastases”, Cell Death Differ., 2020, 27: 161-175; Ding et al., “Structural and Functional Insights into Host Death Domains Inactivation by the Bacterial Arginine GlcNAcyltransferase Effector”, Mol Cell, 2019, 74: 922; Yoshikawa et al., “Discovery of 7-Oxo-2,4,5,7-tetrahydro-6H-pyrazolo[3,4-c]pyridine Derivatives as Potent, Orally Available, and Brain-Penetrating Receptor Interacting Protein 1 (RIP1) Kinase Inhibitors: Analysis of Structure-Kinetic Relationships”, J Med Chem., 2018, 61: 2384-2409; Pierotti et al., “Potent Inhibition of Necroptosis by Simultaneously Targeting Multiple Effectors of the Pathway”, ACS Chem Biol., 2020, 15: 2702-2713; Rubbelke et al., “Locking mixed-lineage kinase domain-like protein in its auto-inhibited state prevents necroptosis”, Proc Natl Acad Sci USA, 2020, 117: 33272-33281; Xie et al., “Structural Basis of RIP1 Inhibition by Necrostatins”, Structure, 2013, 21: 493-499; Harris et al., “Discovery of Small Molecule RIP1 Kinase Inhibitors for the Treatment of Pathologies Associated with Necroptosis”, ACS Med Chem Lett., 2013, 4: 1238-1243; Harris et al., “DNA-Encoded Library Screening Identifies Benzo[b][1,4]oxazepin-4-ones as Highly Potent and Monoselective Receptor Interacting Protein 1 Kinase Inhibitors”, J Med Chem., 2016, 59: 2163-2178; Harris et al., “Discovery and Lead-Optimization of 4,5-Dihydropyrazoles as Mono-Kinase Selective, Orally Bioavailable and Efficacious Inhibitors of Receptor Interacting Protein 1 (RIP1) Kinase”, J Med Chem., 2019, 62: 5096-5110; Harris et al., “Discovery of a First-in-Class Receptor Interacting Protein 1 (RIP1) Kinase Specific Clinical Candidate (GSK2982772) for the Treatment of Inflammatory Diseases”, J Med Chem., 2017, 60: 1247-1261; Harris et al., “Identification of a RIP1 Kinase Inhibitor Clinical Candidate (GSK3145095) for the Treatment of Pancreatic Cancer”, ACS Med Chem Lett, 2019, 10: 857-862; Wang et al., “RIP1 Kinase Drives Macrophage-Mediated Adaptive Immune Tolerance in Pancreatic Cancer”, Cancer Cell, 2018, 34: 757-774.e7.

FIG. 8 presents non-limiting examples of ligands that bind to DNA mismatch repair protein Msh2 (MSH2, MutS protein homolog 2) in the MSH2-MSH6 complex, including the ligands identified in Vasilyeva et al. DNA Repair, 2009, 8(1): 103-113 and Nair et al. Nucleic Acids Res., 2018, 42: 256-266.

FIG. 9A and FIG. 9B present non-limiting examples of ligands that bind to Cyclin-dependent kinase inhibitor 1B (Cyclin-dependent kinase inhibitor p27, CDKN1B, p27Kip1). For additional non-limiting examples and related ligands, see ligands identified by Frankel et al. J. Biol. Chem. 2008, 283(2): 1026-1033 and Iconaru et al. Sci. Rep. 2015, 5: 15686.

FIG. 10 presents a non-limiting example of a ligand that binds to retinal-specific phospholipid-transporting ATPase ABCA4 (ABCA4, RIM ABC transporter, ATP-binding cassette sub-family A member 4, Stargardt disease protein) including AJP and CLR. For additional non-limiting examples and related ligands, see Liu et al. eLife, 2021, 10: e63524.

FIG. 11A and FIG. 11B present non-limiting examples of ligands that bind to bile salt export pump (ABCB11, ATP-binding cassette sub-family B member 11). For additional non-limiting examples and related ligands, see ligands identified by Ritschel et al., Chem. Res. Toxicol., 2014, 27, 873-881 and Jain et al. J. Comput. Aided Mol. Des. 2017, 31(6): 507-521.

FIG. 12 presents non-limiting examples of ligands that bind to Choline O-acetyltransferase (ChAT, choline acetylase, CHOACTase), including the compound RMW. For additional non-limiting examples and related ligands, see ligands identified by Wiktelius et al. Angew. Chem. Int. Ed. 2021, 60(2): 813-819 and Kim et al. Biochemistry, 2006, 45(49), 14621-14631.

FIG. 13 presents a non-limiting example of a ligand that binds to ubiquitin carboxyl-terminal hydrolyase CYLD (CYLD, deubiquitinating enzyme CYLD, ubiquitin-specific-processing protease CYLD), as identified in Yamanaka et al. Biochem. Biophys. Res. Commun., 2020, 524(1): 1-7.

FIG. 14 presents non-limiting examples of ligands that bind to NF-kappa-B essential modulator (NEMO, FIP-3, IkB kinase-associated protein 1, IKKAP1, IKKG). For additional non-limiting examples and related ligands, see ligands identified by Vincendeau et al., Sci. Rep., 2016, 6: 1894 and De Falco et al. Biochemical Pharmacology, 2016, 104: 83-94.

FIG. 15A and FIG. 15B present non-limiting examples of ligands that bind to AH receptor-interacting protein (AIP, Aryl-hydrocarbon receptor-interacting protein, HBV X-associated protein 2). For additional non-limiting examples and related ligands, see ligands identified by Schmees et al. AACR Annual Meeting 2019, Atlanta, GA, Boitano et al., Science, 2010, 329(5997): 1345-1348, Fukuda et al., Biochem. Biophys. Res. Commun., 2007, 359(3): 822-827, Mukai et al., Archives of Biochemistry and Biophysics, 2010, 501: 134-141, and Smith et al., J. Investig. Dermatol., 2017, 137(10): 2110-2119.

FIG. 16 presents non-limiting examples of ligands that binds to programmed cell death protein 4 (PDCD4). For additional non-limiting examples and related ligands, see ligands identified in Frankel et al., J. Biol. Chem. 2008, 283(2): 1026-1033 and Wang et al., “Targeting Programmed Cell Death 4 (PDCD4) with Biogenic Compounds in ARDS by Gaussian Process-Based QSAR Virtual Screening” Journal of Chemometrics 2016, 30: 621-627.

FIG. 17A, FIG. 17B, FIG. 17C, and FIG. 17D present non-limiting examples of ligands that binds to Receptor-interacting serine/threonine-protein kinase 2 (RIPK2) including OLI, E7N, 9WS, 9XA, BW8, KRE, GEZ, Q9J, M5W, M2B, 6GD, 6GE, K9T, KA2, SB2, IQ7, ACP, XYW, and SR8. For additional non-limiting examples and related ligands, see ligands identified in Hrdinka et al. Small molecule inhibitors reveal an indispensable scaffolding role of RIPK2 in NOD2 signaling. (2018) EMBO J 37. He et al. Identification of Potent and Selective RIPK2 Inhibitors for the Treatment of Inflammatory Diseases. (2017) ACS Med Chem Lett 8: 1048-1053. Canning et al. Inflammatory Signaling by NOD-RIPK2 Is Inhibited by Clinically Relevant Type II Kinase Inhibitors. (2015) Chem Biol 22: 1174-1184. Suubsuwong, et al. Activation loop targeting strategy for design of receptor-interacting protein kinase 2 (RIPK2) inhibitors. (2018) Bioorg Med Chem Lett 28: 577-583. Suebsuwong, et al. Design of 3,5-diaryl-2-aminopyridines as receptor-interacting protein kinase 2 (RIPK2) and nucleotide-binding oligomerization domain (NOD) cell signaling inhibitors. Unpublished. Haile, et al. Identification of Quinoline-Based RIP2 Kinase Inhibitors with an Improved Therapeutic Index to the hERG Ion Channel. (2018) ACS Med Chem Lett 9: 1039-1044. Haffner, et al. Discovery of Pyrazolocarboxamides as Potent and Selective Receptor Interacting Protein 2 (RIP2) Kinase Inhibitors. (2019) ACS Med Chem Lett 10: 1518-1523. Pellegrini, et al. Structures of the inactive and active states of RIP2 kinase inform on the mechanism of activation. (2017) PLoS One 12: e0177161-e0177161. Charnley, et al. Crystal Structures of Human Rip2 Kinase Catalytic Domain Complexed with ATP-Competitive Inhibitors: Foundations for Understanding Inhibitor Selectivity. (2015) Bioorg Med Chem 23: 7000

FIG. 18A, FIG. 18B, and FIG. 18C present non-limiting examples of ligands that binds to apoptosis regulator BAX. For additional non-limiting examples and related ligands, see Li et. al U.S. Pat. No. 9,561,215, Halazy, et al. Preparation of 9-(piperazinylalkyl) carbazoles as Bax-modulators WO2001/029028. Halazy et al, Synthesis of substituted N-acyl/sulfonyl pyrrolidine derivatives as bax inhibitors. WO2001/072705A1. Halazy, et al. Preparation of pyrrolidines as inhibitors of Bax function. WO2001/074769A1. Xingming et al. Preparation of fluoren-9-ylidenemethylpyridine derivatives as Bax agonists WO2013/028543A1. Walensky et al. Preparation of pyrazol-3-ones as activators of pro-apoptotic BAX. WO2013055949A2. Gavathiotis, et al. Direct and selective small-molecule activation of proapoptotic BAX. Nature Chemical Biology 8, 639-645 (2012). Garner et al. Small-molecule allosteric inhibitors of BAX. Nat Chem Biol 15, 322-330 (2019). Stornaiuolo et al. Structure-Based Lead Optimization and Biological Evaluation of BAX Direct Activators as Novel Potential Anticancer Agents J. Med. Chem. 2015, 58, 5, 2135-2148. Spitz et al. Eltrombopag directly inhibits BAX and prevents cell death. Nature Communications 12, 1134 (2021). Reyna et al. Direct Activation of BAX by BTSA1 Overcomes Apoptosis Resistance in Acute Myeloid Leukemia. Cancer Cell 32, 490-505.e10 (2017).

FIG. 19A and FIG. 19B present a non-limiting example of ligands that bind to P21 (CDKN1A, P21Cip1/Waf1, CAP20, Cyclin-Dependent Kinase Inhibitor 1A). For additional non-limiting examples and related ligands, see Weiss et al. US 2015/0132408, Weiss et al. WO 2014/007998, Park et al. High throughput screening of a small molecule one-bead-one-compound combinatorial library to identify attenuators of p21 as chemotherapy sensitizers. Cancer Biology & Therapy, (7), 12, 2015-2022, and Weiss et al. US 2011/0301192.

FIG. 20 presents a non-limiting example of ligands that bind to alpha-1-antitrypsin (AAT, SERPINA1). For additional non-limiting examples, see Smith et al. WO2019/243841. Mallya et al. Small Molecules Block the Polymerization of Z al-Antitrypsin and Increase the Clearance of Intracellular Aggregates. J. Med. Chem. (2007), 50(22), 5357-5363. Patschull, et al. In silico assessment of potential druggable pockets on the surface of al-antitrypsin conformers PLoS One (2012), 7(5), e36612

FIG. 21A, FIG. 21B, and FIG. 21C present non-limiting examples of ligands that bind to pyruvate kinase liver/red blood cell (Pyruvate kinase L/R, PKLR). For additional non-limiting examples, see WO 2019/035863, WO 2019/035863, WO2020198067, and WO2019/075367.

FIG. 22 presents a non-limiting example of ligands that bind to Kelch-like ECH-associated protein 1 (KEAP1). For additional non-limiting examples, see Tran et al. A Comparative Assessment Study of Known Small-Molecule Keap1-Nrf2 Protein-Protein Interaction Inhibitors: Chemical Synthesis, Binding Properties, and Cellular Activity. J Med Chem 62, 8028-8052 (2019).

FIG. 23 presents a non-limiting example of ligands that bind to Phosphatase and Tensin Homolog (PTEN). For additional non-limiting examples, see Li et al. Pretreatment with phosphatase and tensin homolog deleted on chromosome 10 (PTEN) inhibitor SF1670 augments the efficacy of granulocyte transfusion in a clinically relevant mouse model. Blood (2011) 117 (24): 6702-6713.

FIG. 24 presents a non-limiting example of ligands that bind to Interleukin 1 Receptor Associated Kinase 4 (IRAK4). For additional non-limiting examples, see McElroy, W. T. Interleukin-1 receptor-associated kinase 4 (IRAK4) inhibitors: an updated patent review (2016-2018). Expert Opin Ther Pat 29, 243-259 (2019); Lee et al. J. Med. Chem. 2017, 60, 13, 5521-5542, WO 2017205762A1, WO 2017205766A1, WO 2017205769A1

FIG. 25A and FIG. 25B present non-limiting examples of ligands that bind to Thymidine kinase 2, mitochondrial (TK2). For additional non limiting examples, see Van Poeke et al. 3′-[4-Aryl-(1,2,3-triazol-1-yl)]-3′-deoxythymidine Analogues as Potent and Selective Inhibitors of Human Mitochondrial Thymidine Kinase J. Med. Chem. 2010, 53, 7, 2902-2912; Kierdaszuk et al. Substrate/Inhibitor Properties of Human Deoxycitidine Kinase (dCK) and Thymidine Kinases (Tk1 and Tk2) Towards the Sugar Moiety of Nucleosides, Including O′-Alkyl Analogues Nucleosides Nucleotides Nucleic Acids 1999, 18, 1883-1903; and Priego et al. Recent Advances in Thymidine Kinase 2 (TK2) Inhibitors and New Perspectives for Potential Applications. Current Pharmaceutical Design, 2012, 18, 2981-2994

FIG. 26 presents a non-limiting example of ligands that bind to Potassium Voltage-Gated Channel Subfamily Q Member 1 (KCNQ1). For additional non-limiting examples, see Mattmann Identification of (R)—N-(4-(4-methoxyphenyl)thiazol-2-yl)-1-tosylpiperidine-2-carboxamide, ML277, as a novel, potent and selective Kv7.1 (KCNQ1) potassium channel activator. Bioorg Med Chem Lett. 2012 Sep. 15; 22(18): 5936-5941; Salata, J. et al. A Novel Benzodiazapine that Activated Cardiac Slow Delayed Rectifier K+ Currents. Molecular Pharmacology. 1998, 53, 220; Abbott, G. KCNQs: Ligand- and Voltage-Gated Potassium Channels. Front. Physiol. 2020, 11, 583.

FIG. 27 presents a non-limiting example of ligands that bind to Stimulator of Interferon Genes (transmembrane protein 173, ERIS, MITA, TMEM173, encoded by gene STING1). For additional non-limiting examples, see Pryde, D. C. et al. The discovery of potent small molecule activators of human STING. Eur J Med Chem 209, 112869 (2021); Ramanjulu, J. M. et al. Design of amidobenzimidazole STING receptor agonists with systemic activity. Nature 564, 439-443 (2018).

FIG. 28 provides non-limiting examples of compounds of the present invention.

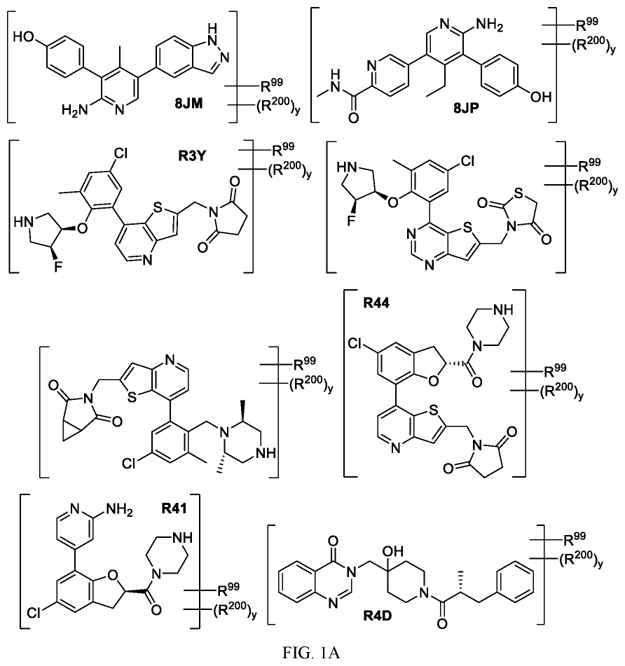

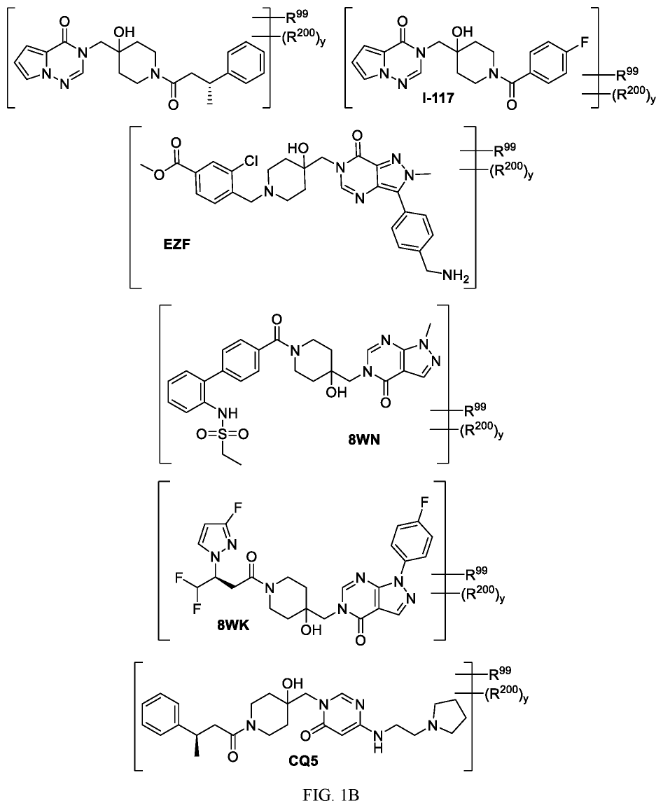

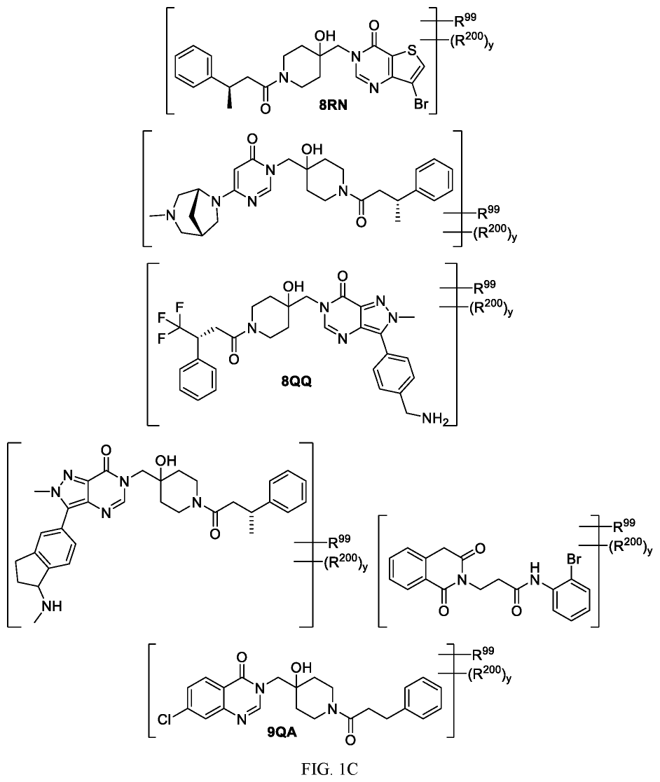

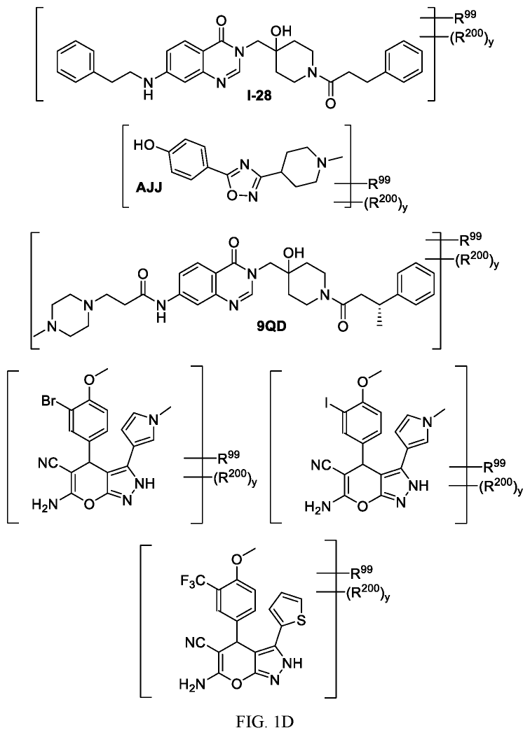

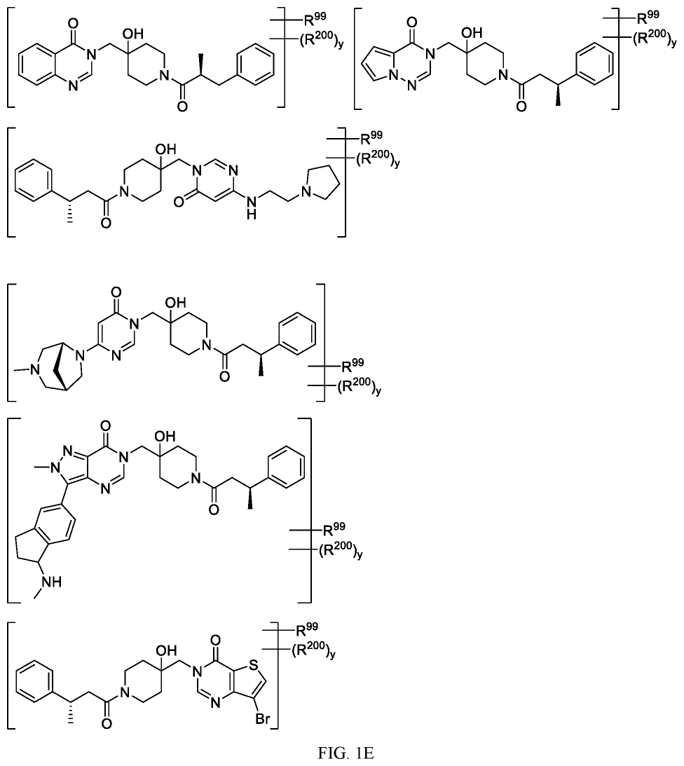

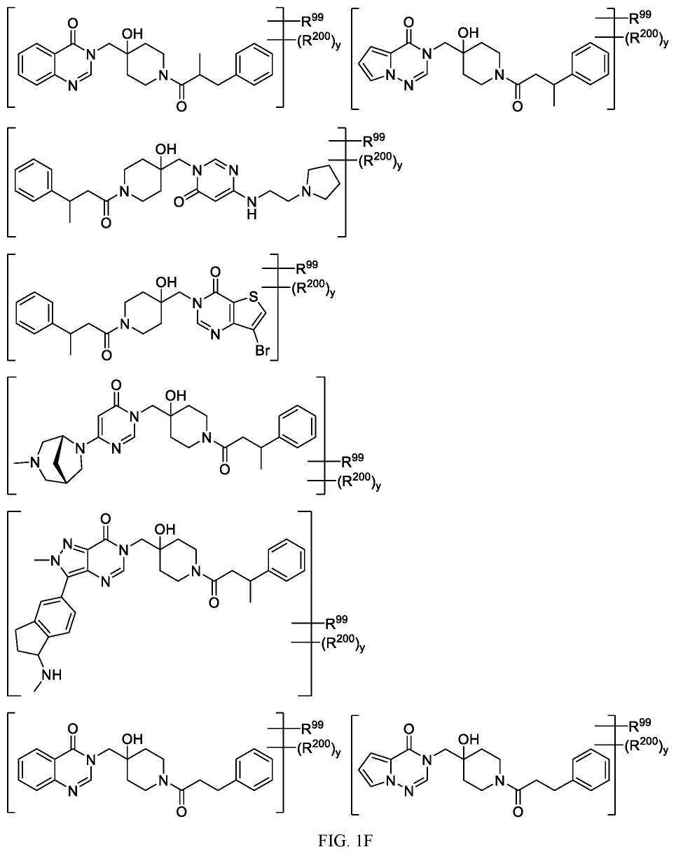

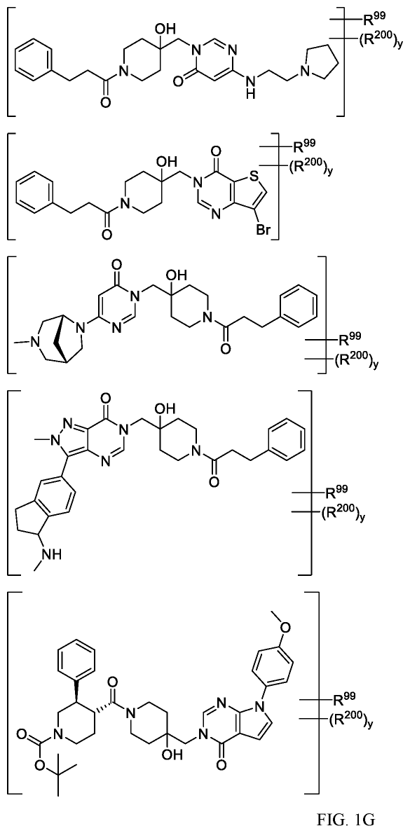

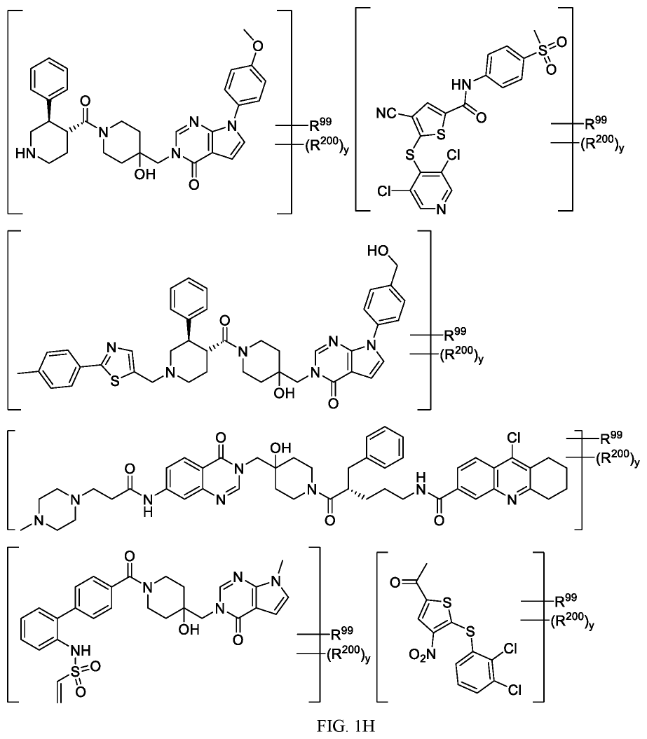

### DETAILED DESCRIPTION OF THE INVENTION

Protein stabilizing and/or function restoring compounds and their uses and manufacture are provided that stabilize a Target Ubiquitinated Protein by deubiquitinating it and in some embodiments restore at least a partial amount of the protein's function. The protein stabilizing and/or function restoring compounds described herein include a USP7 Targeting Ligand, a Ubiquitinated Protein Targeting Ligand, and optionally a Linker. In some embodiments, the protein's function is restored by at least about 1%, 2.5%, 5%, 7.5%, 10%, 15% or more over the native protein or a mutated or altered form of the protein, as relevant in context.

When a deubiquitinase removes ubiquitins from a protein the proteasomal degradation of the protein may be prevented (i.e. the protein is stabilized), the protein may resume its activity (i.e. the protein's function is restored), or the deubiquitination may be insufficient to prevent degradation or restore function. A compound described herein removes ubiquitin from the Target Ubiquitinated Protein in a manner that stabilizes the protein and in some embodiments restore the protein's function (for example restoring at least about 1%, 2.5%, 5%, 7.5%, 10%, 15%, 20%, 25%, 30%, 40%, 50%, 60%, 70%, 80%, 90%, or 100% protein function). By both stabilizing and restoring the protein's function various disorders that are caused by a deficiency of a protein's activity can be treated. For example, disorders caused by loss of function protein mutations or haploid insufficiency can be treated by restoring the function of the downregulated wildtype protein or interest or a mutant thereof. Difficult to treat cancers can also be treated with a protein stabilizing compound of the present invention. For example, cancers that downregulate tumor suppressors can be treated by restoring the function of the tumor suppressor. A protein stabilizing compound described herein can also prompt an immunological response in the treatment of cancer and thus treat the cancer by activating the immune system.

The protein stabilizing compound as described herein in principle embodiments has a stable shelf life for at least 2 months, 3 months, 6 months or 1 year or more neat or as part of a pharmaceutically acceptable dosage form, and itself is pharmaceutically acceptable.

### Embodiments of Formula I

In certain embodiments the protein stabilizing compound of the present invention is selected from:

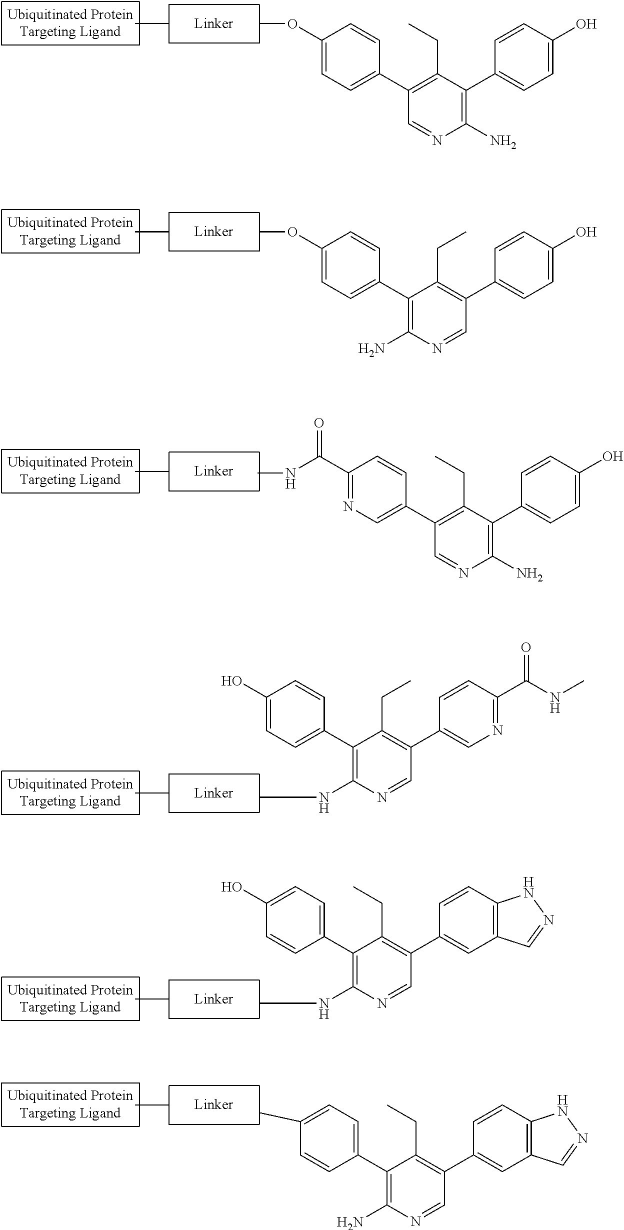

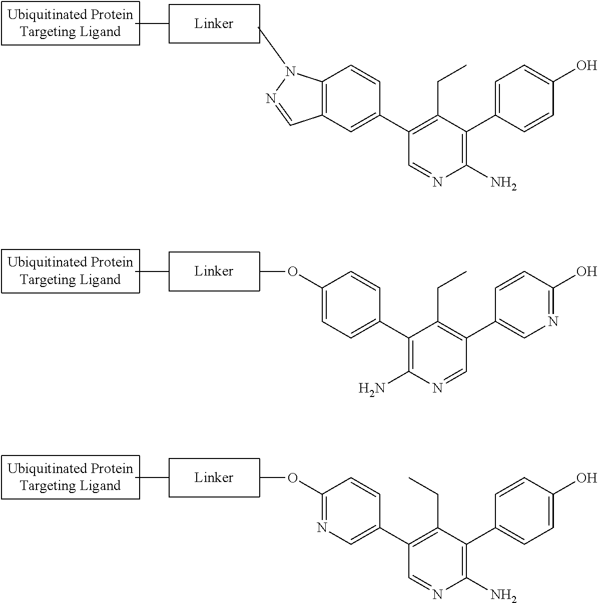

or a pharmaceutically acceptable salt thereof.

In certain embodiments the protein stabilizing compound of the present invention is selected from:

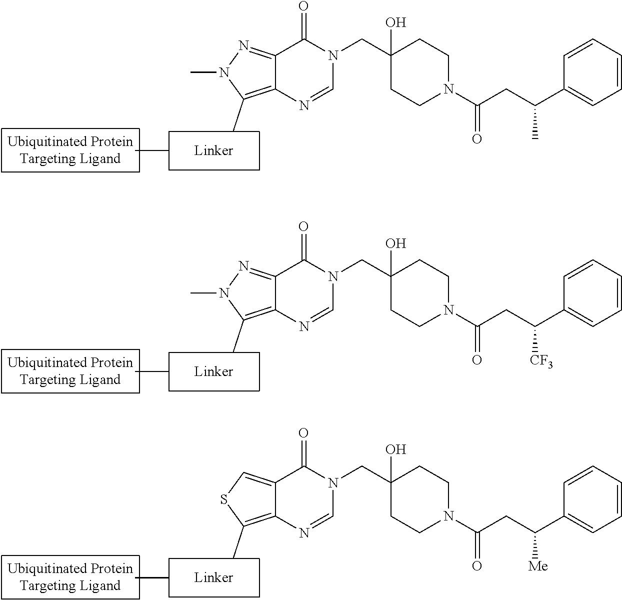

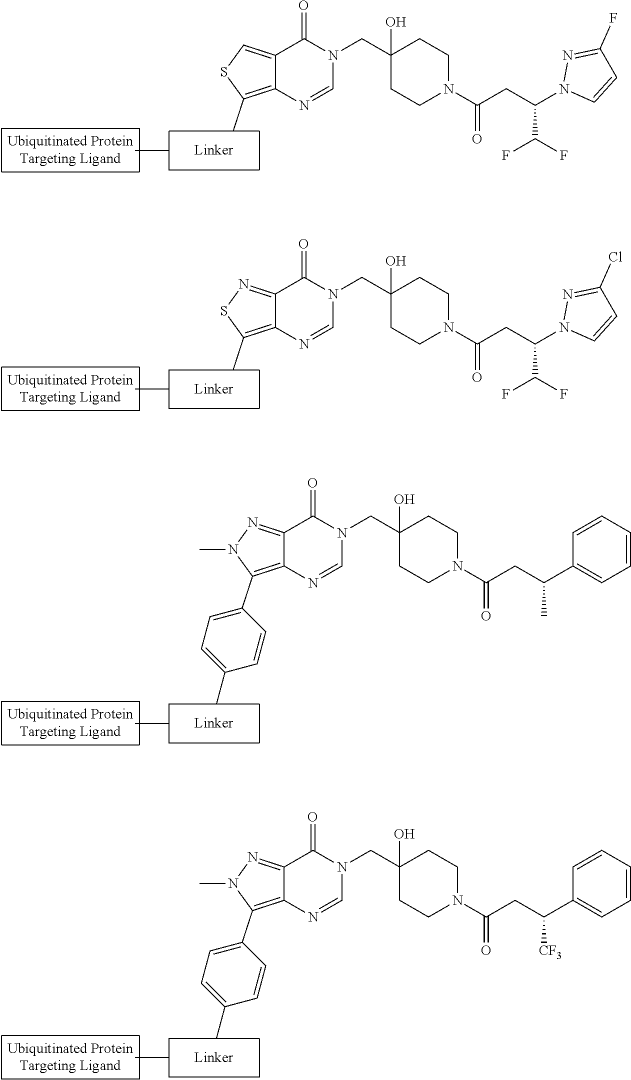

or a pharmaceutically acceptable salt thereof.

In certain embodiments the protein stabilizing compound of the present invention is selected from:

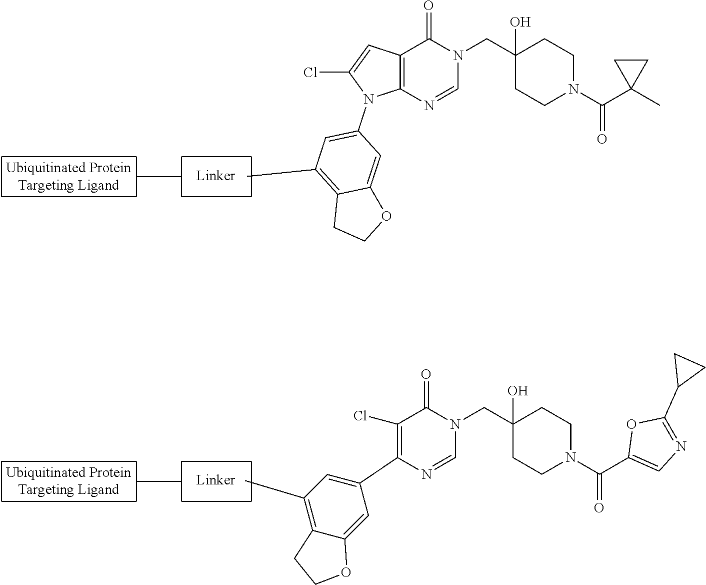

or a pharmaceutically acceptable salt thereof.

In certain embodiments the protein stabilizing compound of the present invention is selected from:

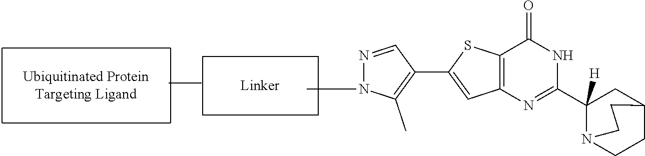

or a pharmaceutically acceptable salt thereof.

In certain embodiments the protein stabilizing compound of the present invention is selected from:

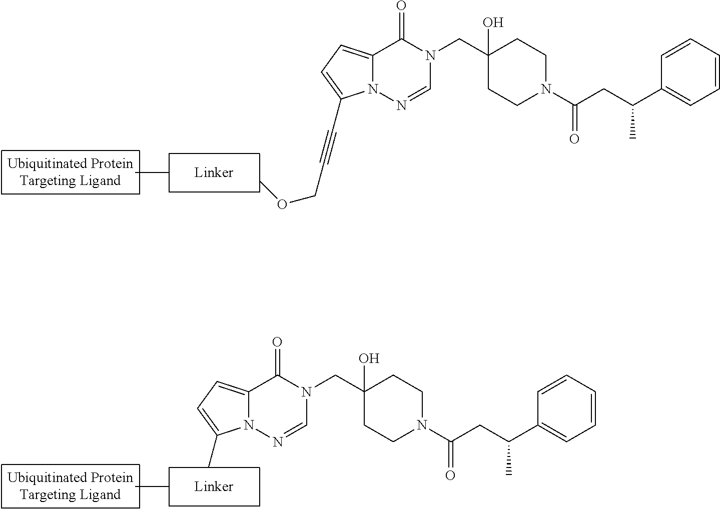

or a pharmaceutically acceptable salt thereof.

In certain embodiments the protein stabilizing compound of the present invention is selected from:

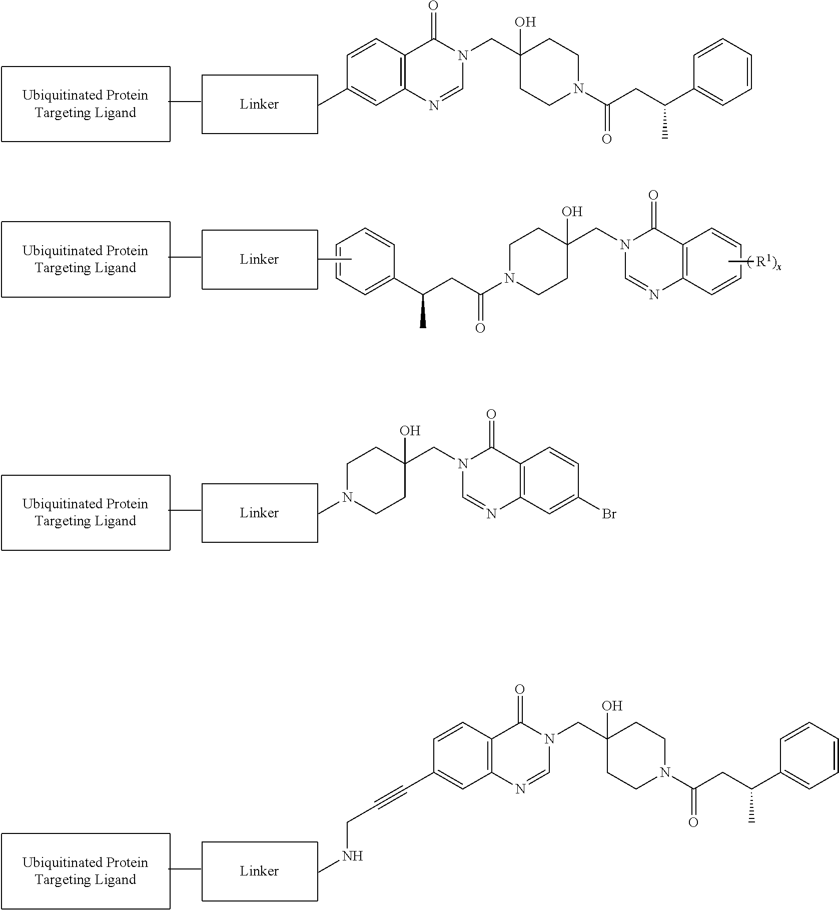

or a pharmaceutically acceptable salt thereof.

In certain embodiments the protein stabilizing compound of the present invention is selected from:

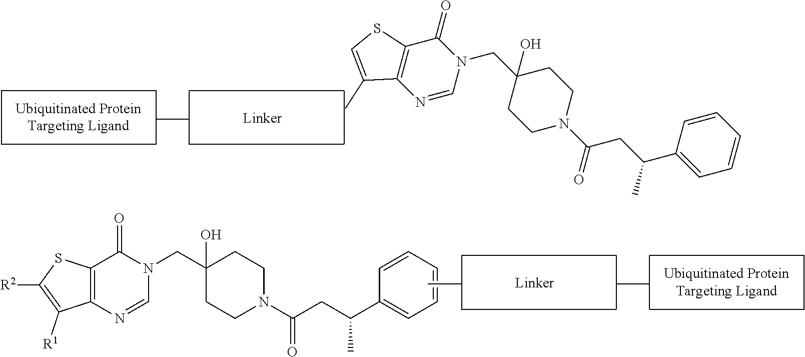

or a pharmaceutically acceptable salt thereof.

In certain embodiments the protein stabilizing compound of the present invention is selected from:

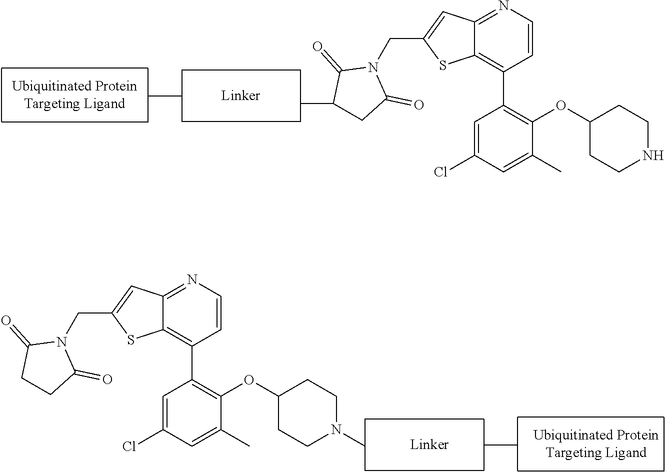

or a pharmaceutically acceptable salt thereof.

In certain embodiments the protein stabilizing compound of the present invention is selected from:

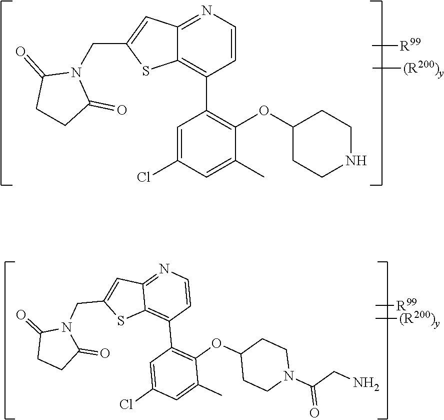

or a pharmaceutically acceptable salt thereof.
wherein:
R⁹⁹ is the attachment point to Linker-Ubiquitinated Protein Targeting Ligand;
R¹⁰⁰ is the attachment point to Linker-USP7 Targeting Ligand; and
R²⁰⁰ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹.

In certain embodiments the protein stabilizing compound of the present invention is selected from:

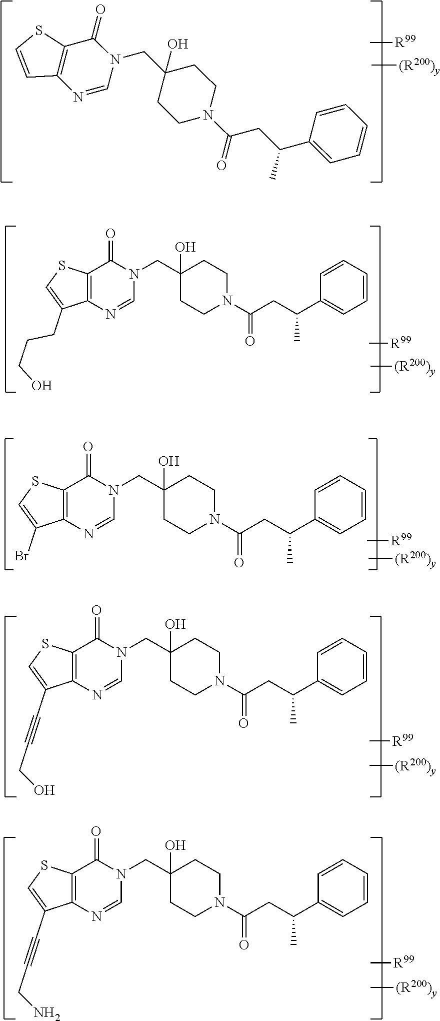

or a pharmaceutically acceptable salt thereof.

In certain embodiments the protein stabilizing compound of the present invention is selected from:

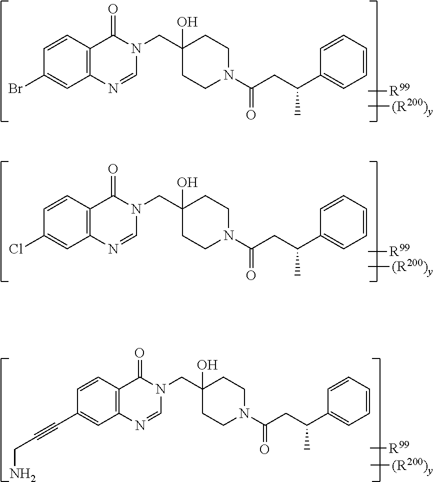

or a pharmaceutically acceptable salt thereof.

In certain embodiments the protein stabilizing compound of the present invention is

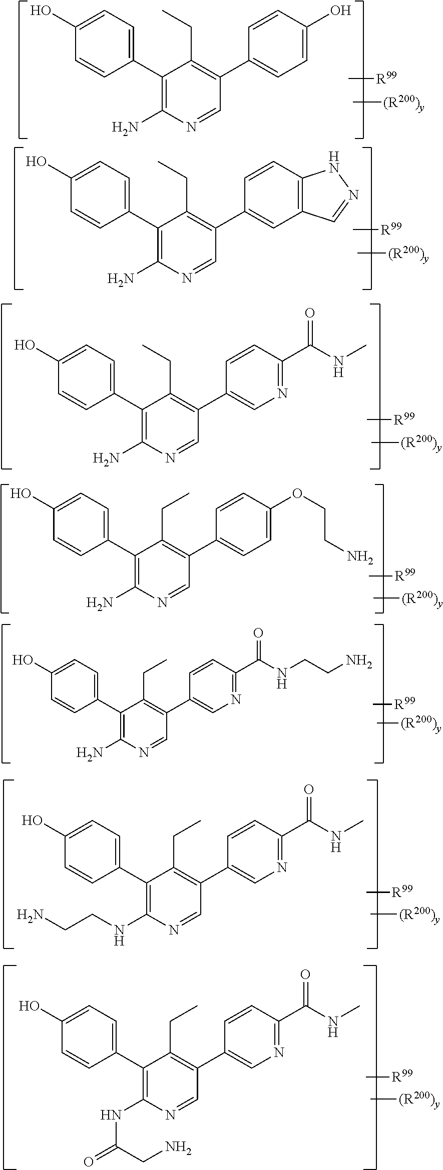

or a pharmaceutically acceptable salt thereof.

In certain embodiments the protein stabilizing compound of the present invention is selected from:

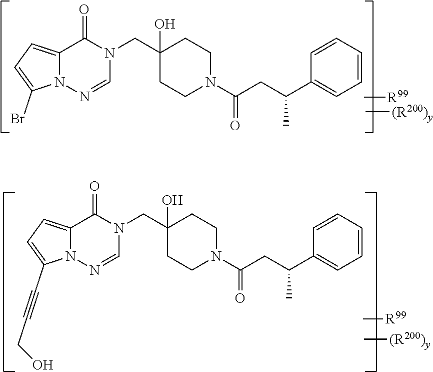

or a pharmaceutically acceptable salt thereof.

In certain embodiments the protein stabilizing compound of the present invention is selected from:

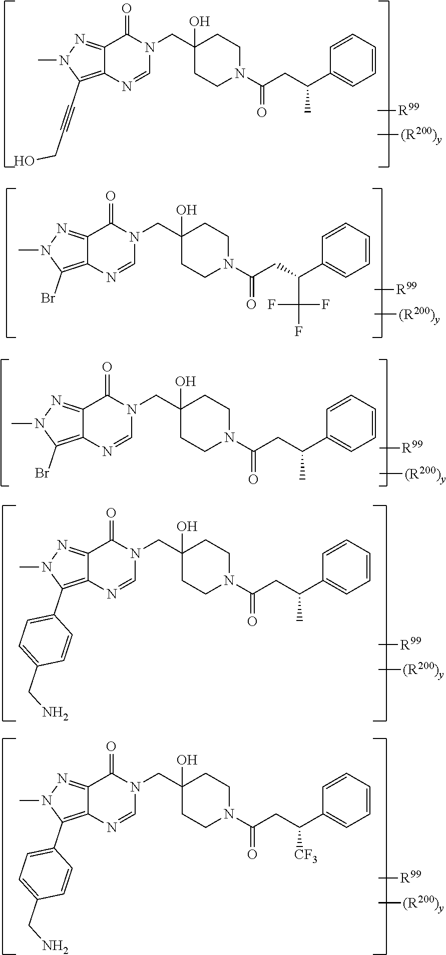

or a pharmaceutically acceptable salt thereof.

In certain embodiments the protein stabilizing compound of the present invention is selected from:

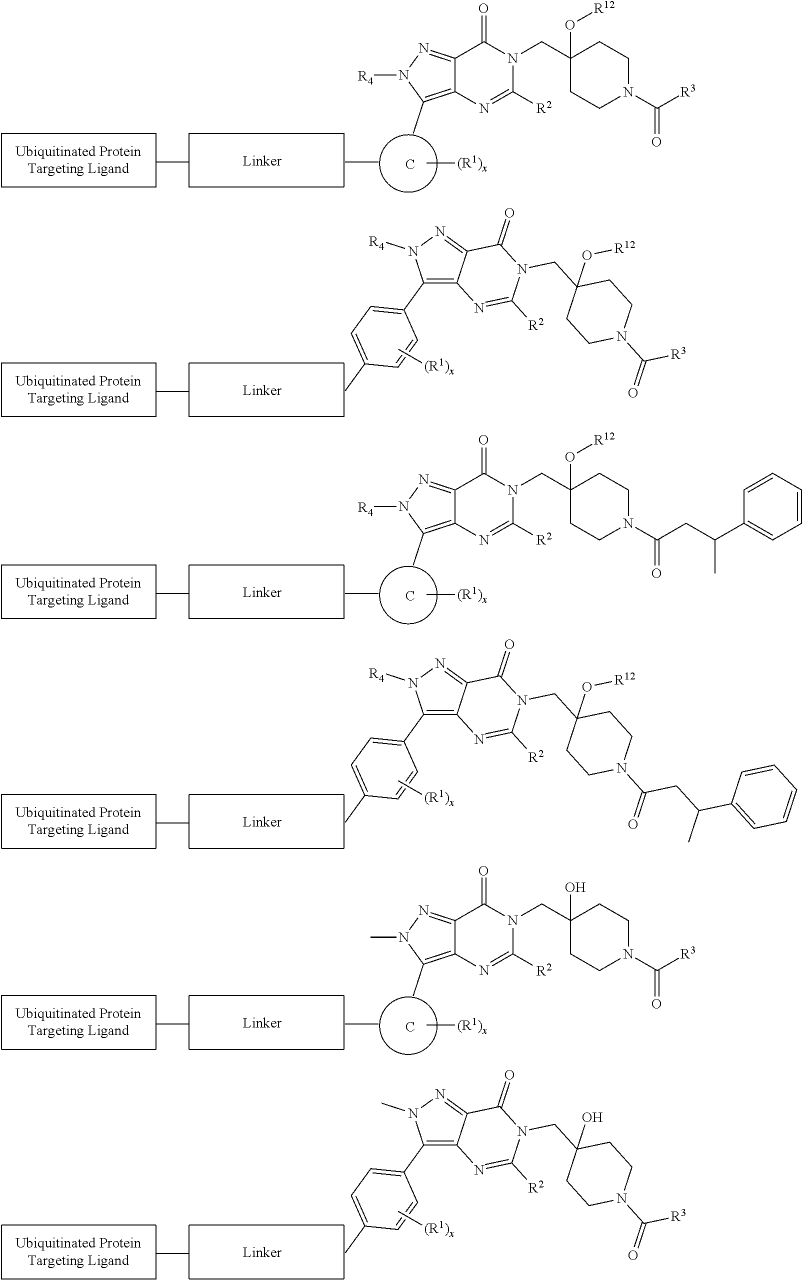

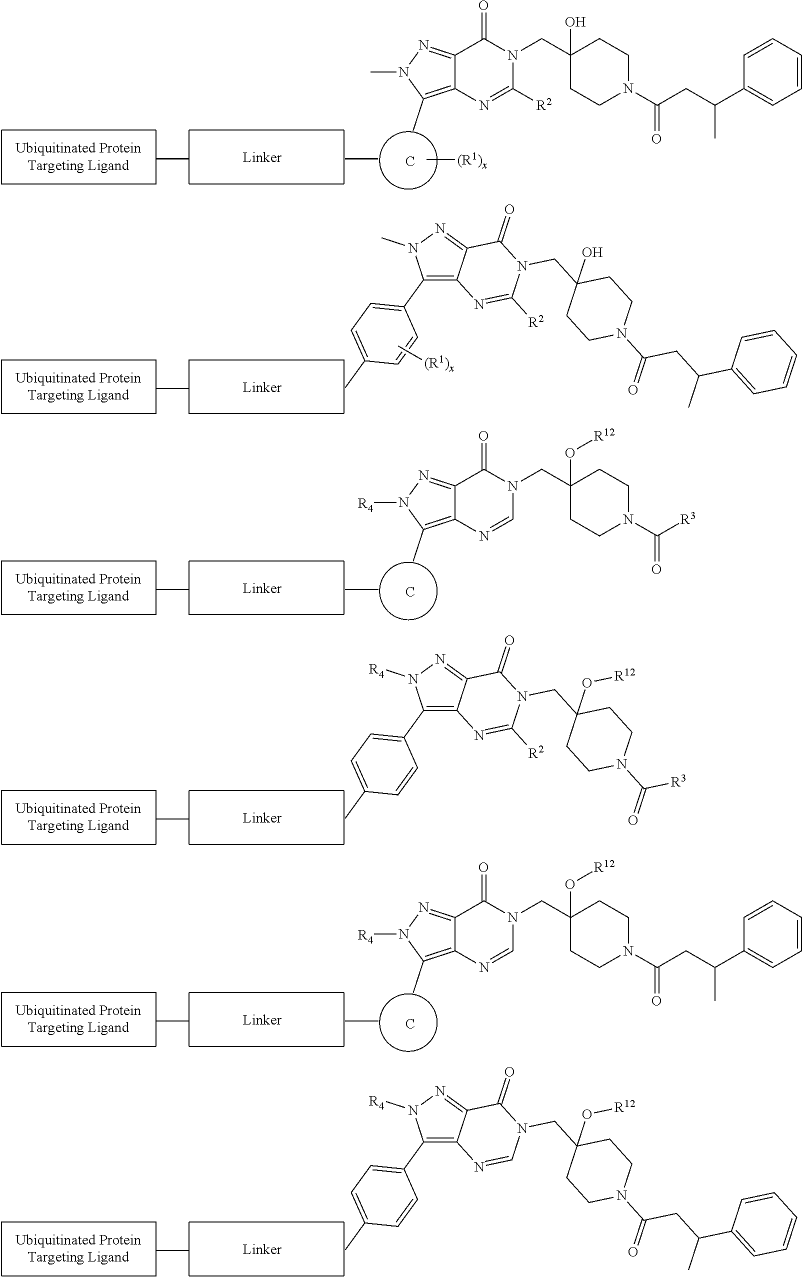

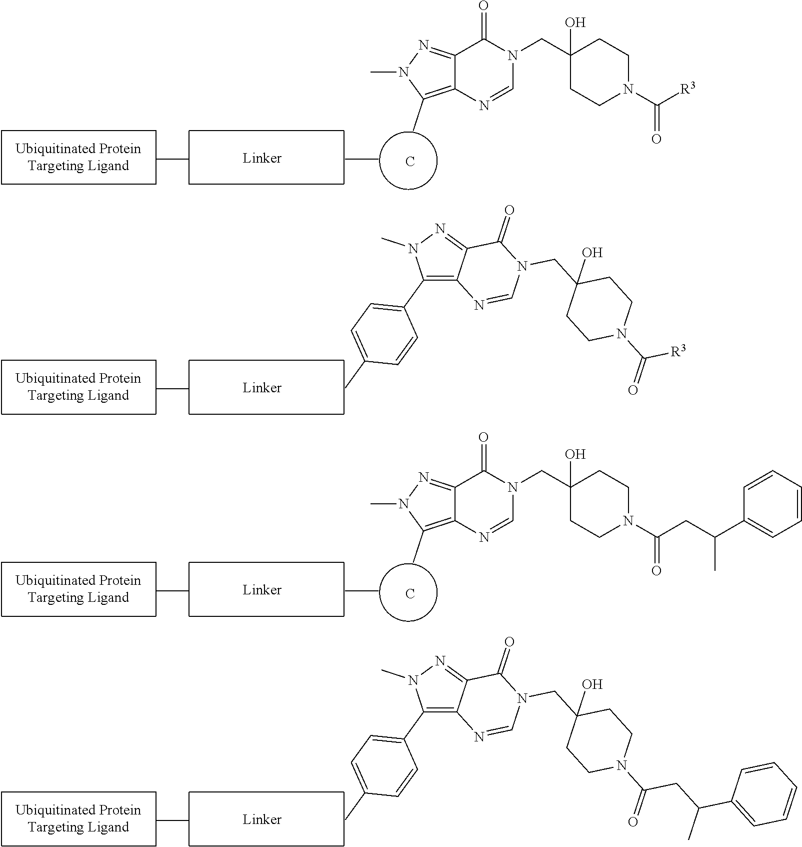

or a pharmaceutically acceptable salt thereof.

In certain embodiments the protein stabilizing compound of the present invention is selected from:

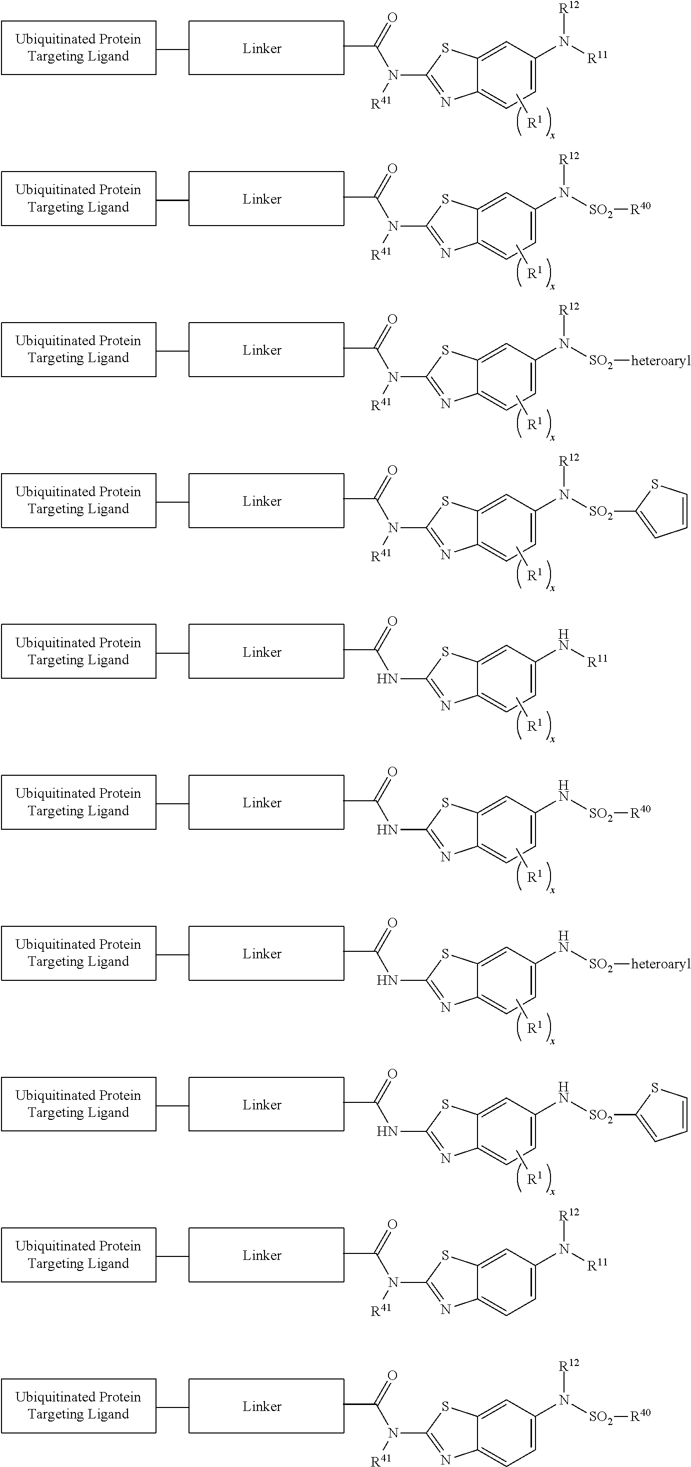

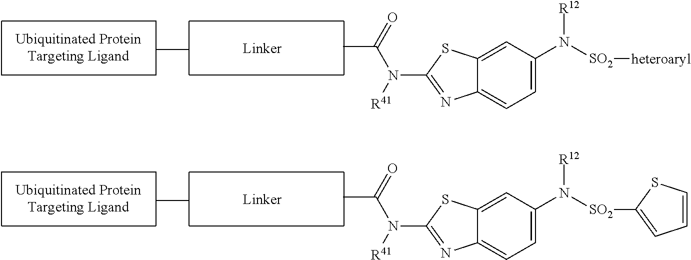

or a pharmaceutically acceptable salt thereof

### Embodiments of

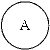

In certain embodiments

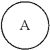

is an aryl group.

In certain embodiments

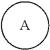

is a phenyl group.

In certain embodiments

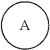

is a heteroaryl group.

In certain embodiments

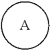

is a heterocycle group.

In certain embodiments

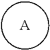

is a cycloalkyl group.

### Embodiments of

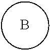

In certain embodiments

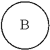

is an aryl group.

In certain embodiments

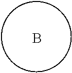

is a phenyl group.

In certain embodiments

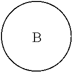

is a heteroaryl group.

In certain embodiments

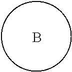

is a heterocycle group.

In certain embodiments

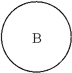

is a cycloalkyl group.

### Embodiments of

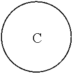

In certain embodiments

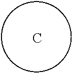

is an aryl group.

In certain embodiments

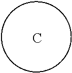

is a phenyl group.

In certain embodiments

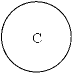

is a heteroaryl group.

In certain embodiments

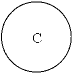

is a heterocycle group.

In certain embodiments

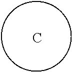

is a cycloalkyl group.

### Embodiments of x, y, and z

In certain embodiments x is 0.

In certain embodiments x is 1.

In certain embodiments x is 2.

In certain embodiments x is 3.

In certain embodiments x is 4.

In certain embodiments y is 0.

In certain embodiments y is 1.

In certain embodiments y is 2.

In certain embodiments y is 3.

In certain embodiments z is 0.

In certain embodiments z is 1.

In certain embodiments z is 2.

In certain embodiments z is 3.

In certain embodiments z is 4.

### Embodiments of R¹

In certain embodiments a R¹ is hydrogen.

In certain embodiments one R¹ is hydrogen.

In certain embodiments all R¹ groups are hydrogen.

In certain embodiments a R¹ is halogen.

In certain embodiments one R¹ is halogen.

In certain embodiments a R¹ is alkyl optionally substituted with 1, 2, 3, or 4 substituents selected from R²¹.

In certain embodiments one R¹ is alkyl optionally substituted with 1, 2, 3, or 4 substituents selected from R²¹.

In certain embodiments a R¹ is haloalkyl optionally substituted with 1, 2, 3, or 4 substituents selected from R²¹.

In certain embodiments one R¹ is haloalkyl optionally substituted with 1, 2, 3, or 4 substituents selected from R²¹.

In certain embodiments a R¹ is alkenyl optionally substituted with 1, 2, 3, or 4 substituents selected from R²¹.

In certain embodiments one R¹ is alkenyl optionally substituted with 1, 2, 3, or 4 substituents selected from R²¹.

In certain embodiments a R¹ is alkynyl optionally substituted with 1, 2, 3, or 4 substituents selected from R²¹.

In certain embodiments one R¹ is alkynyl optionally substituted with 1, 2, 3, or 4 substituents selected from R²¹.

In certain embodiments a R¹ is heterocycle optionally substituted with 1, 2, 3, or 4 substituents selected from R²¹.

In certain embodiments one R¹ is heterocycle optionally substituted with 1, 2, 3, or 4 substituents selected from R²¹.

In certain embodiments a R¹ is aryl optionally substituted with 1, 2, 3, or 4 substituents selected from R²¹.

In certain embodiments one R¹ is aryl optionally substituted with 1, 2, 3, or 4 substituents selected from R²¹.

In certain embodiments a R¹ is heteroaryl optionally substituted with 1, 2, 3, or 4 substituents selected from R²¹.

In certain embodiments one R¹ is heteroaryl optionally substituted with 1, 2, 3, or 4 substituents selected from R²¹.

In certain embodiments a R¹ is cyano.

In certain embodiments one R¹ is cyano.

In certain embodiments a R¹ is nitro.

In certain embodiments one R¹ is nitro.

In certain embodiments a R¹ is —C(O)R¹⁰.

In certain embodiments one R¹ is —C(O)R¹⁰.

In certain embodiments a R¹ is —OC(O)R¹⁰.

In certain embodiments one R¹ is —OC(O)R¹⁰.

In certain embodiments a R¹ is —NR¹¹C(O)R¹⁰.

In certain embodiments one R¹ is —NR¹¹C(O)R¹⁰.

In certain embodiments a R¹ is —OR¹¹.

In certain embodiments one R¹ is —OR¹¹.

In certain embodiments a R¹ is —NR¹¹R¹².

In certain embodiments one R¹ is —NR¹¹R1.

In certain embodiments a R¹ is —S(O)R¹⁰.

In certain embodiments one R¹ is —S(O)R¹⁰.

In certain embodiments a R¹ is —S(O)₂R¹⁰.

In certain embodiments one R¹ is —S(O)₂R¹⁰.

In certain embodiments a R¹ is —OS(O)R¹⁰.

In certain embodiments one R¹ is —OS(O)R¹⁰.

In certain embodiments a R¹ is —OS(O)₂R¹⁰.

In certain embodiments one R¹ is —OS(O)₂R¹⁰.

In certain embodiments a R¹ is —NR¹¹S(O)R¹⁰.

In certain embodiments one R¹ is —NR¹¹S(O)R¹⁰.

In certain embodiments a R¹ is —NR¹¹S(O)₂R¹⁰.

In certain embodiments one R¹ is —NR¹¹S(O)₂R¹⁰.

In certain embodiments a R¹ is —SR¹¹.

In certain embodiments one R¹ is —SR¹¹.

### Embodiments of R²

In certain embodiments a R² is hydrogen.

In certain embodiments one R² is hydrogen.

In certain embodiments all R² groups are hydrogen.

In certain embodiments a R² is halogen.

In certain embodiments one R² is halogen.

In certain embodiments a R² is alkyl optionally substituted with 1, 2, 3, or 4 substituents selected from R²².

In certain embodiments one R² is alkyl optionally substituted with 1, 2, 3, or 4 substituents selected from R²².

In certain embodiments a R² is haloalkyl optionally substituted with 1, 2, 3, or 4 substituents selected from R²².

In certain embodiments one R² is haloalkyl optionally substituted with 1, 2, 3, or 4 substituents selected from R²².

In certain embodiments a R² is alkenyl optionally substituted with 1, 2, 3, or 4 substituents selected from R²².

In certain embodiments one R² is alkenyl optionally substituted with 1, 2, 3, or 4 substituents selected from R²².

In certain embodiments a R² is alkynyl optionally substituted with 1, 2, 3, or 4 substituents selected from R²².

In certain embodiments one R² is alkynyl optionally substituted with 1, 2, 3, or 4 substituents selected from R²².

In certain embodiments a R² is heterocycle optionally substituted with 1, 2, 3, or 4 substituents selected from R²².

In certain embodiments one R² is heterocycle optionally substituted with 1, 2, 3, or 4 substituents selected from R²².

In certain embodiments a R² is aryl optionally substituted with 1, 2, 3, or 4 substituents selected from R²².

In certain embodiments one R² is aryl optionally substituted with 1, 2, 3, or 4 substituents selected from R²².

In certain embodiments a R² is heteroaryl optionally substituted with 1, 2, 3, or 4 substituents selected from R²².

In certain embodiments one R² is heteroaryl optionally substituted with 1, 2, 3, or 4 substituents selected from R²².

In certain embodiments a R² is cyano.

In certain embodiments one R² is cyano.

In certain embodiments a R² is nitro.

In certain embodiments one R² is nitro.

In certain embodiments a R² is —C(O)R¹⁰.

In certain embodiments one R² is —C(O)R¹⁰.

In certain embodiments a R² is —OC(O)R¹⁰.

In certain embodiments one R² is —OC(O)R¹⁰.

In certain embodiments a R² is —NR¹¹C(O)R¹⁰.

In certain embodiments one R² is —NR¹¹C(O)R¹⁰.

In certain embodiments a R² is —OR¹¹.

In certain embodiments one R² is —OR¹¹.

In certain embodiments a R² is —NR¹¹R¹².

In certain embodiments one R² is —NR¹¹R¹².

In certain embodiments a R² is —S(O)R¹⁰.

In certain embodiments one R² is —S(O)R¹⁰.

In certain embodiments a R² is —S(O)₂R¹⁰.

In certain embodiments one R² is —S(O)₂R¹⁰.

In certain embodiments a R² is —OS(O)R¹⁰.

In certain embodiments one R² is —OS(O)R¹⁰.

In certain embodiments a R² is —OS(O)₂R¹⁰.

In certain embodiments one R² is —OS(O)₂R¹⁰.

In certain embodiments a R² is —NR¹¹S(O)R¹⁰.

In certain embodiments one R² is —NR¹¹S(O)R¹⁰.

In certain embodiments a R² is —NR¹¹S(O)₂R¹⁰.

In certain embodiments one R² is —NR¹¹S(O)₂R¹⁰.

In certain embodiments a R² is —SR¹¹.

In certain embodiments one R² is —SR¹¹.

### Embodiments of R³

In certain embodiments a R³ is hydrogen.

In certain embodiments one R³ is hydrogen.

In certain embodiments all R³ groups are hydrogen.

In certain embodiments a R³ is halogen.

In certain embodiments one R³ is halogen.

In certain embodiments a R³ is alkyl optionally substituted with 1, 2, 3, or 4 substituents selected from R²³.

In certain embodiments one R³ is alkyl optionally substituted with 1, 2, 3, or 4 substituents selected from R²³.

In certain embodiments a R³ is haloalkyl optionally substituted with 1, 2, 3, or 4 substituents selected from R²³.

In certain embodiments one R³ is haloalkyl optionally substituted with 1, 2, 3, or 4 substituents selected from R²³.

In certain embodiments a R³ is alkenyl optionally substituted with 1, 2, 3, or 4 substituents selected from R²³.

In certain embodiments one R³ is alkenyl optionally substituted with 1, 2, 3, or 4 substituents selected from R²³.

In certain embodiments a R³ is alkynyl optionally substituted with 1, 2, 3, or 4 substituents selected from R²³.

In certain embodiments one R³ is alkynyl optionally substituted with 1, 2, 3, or 4 substituents selected from R²³.

In certain embodiments a R³ is heterocycle optionally substituted with 1, 2, 3, or 4 substituents selected from R²³.

In certain embodiments one R³ is heterocycle optionally substituted with 1, 2, 3, or 4 substituents selected from R²³.

In certain embodiments a R³ is aryl optionally substituted with 1, 2, 3, or 4 substituents selected from R²³.

In certain embodiments one R³ is aryl optionally substituted with 1, 2, 3, or 4 substituents selected from R²³.

In certain embodiments a R³ is heteroaryl optionally substituted with 1, 2, 3, or 4 substituents selected from R²³.

In certain embodiments one R³ is heteroaryl optionally substituted with 1, 2, 3, or 4 substituents selected from R²³.

In certain embodiments a R³ is cyano.

In certain embodiments one R³ is cyano.

In certain embodiments a R³ is nitro.

In certain embodiments one R³ is nitro.

In certain embodiments a R³ is —C(O)R¹⁰.

In certain embodiments one R³ is —C(O)R¹⁰.

In certain embodiments a R³ is —OC(O)R¹⁰.

In certain embodiments one R³ is —OC(O)R¹⁰.

In certain embodiments a R³ is —NR¹¹C(O)R¹⁰.

In certain embodiments one R³ is —NR¹¹C(O)R¹⁰.

In certain embodiments a R³ is —OR¹¹.

In certain embodiments one R³ is —OR¹¹.

In certain embodiments a R³ is —NR¹¹R¹².

In certain embodiments one R³ is —NR¹¹R¹².

In certain embodiments a R³ is —S(O)R¹⁰.

In certain embodiments one R³ is —S(O)R¹⁰.

In certain embodiments a R³ is —S(O)₂R¹⁰.

In certain embodiments one R³ is —S(O)₂R¹⁰.

In certain embodiments a R³ is —OS(O)R¹⁰.

In certain embodiments one R³ is —OS(O)R¹⁰.

In certain embodiments a R³ is —OS(O)₂R¹⁰.

In certain embodiments one R³ is —OS(O)₂R¹⁰.

In certain embodiments a R³ is —NR¹¹S(O)R¹⁰.

In certain embodiments one R³ is —NR¹¹S(O)R¹⁰.

In certain embodiments a R³ is —NR¹¹S(O)₂R¹⁰.

In certain embodiments one R³ is —NR¹¹S(O)₂R¹⁰.

In certain embodiments a R³ is —SR¹¹.

In certain embodiments one R³ is —SR¹¹.

### Embodiments of R⁴

In certain embodiments a R⁴ is hydrogen.

In certain embodiments one R⁴ is hydrogen.

In certain embodiments all R⁴ groups are hydrogen.

In certain embodiments a R⁴ is halogen.

In certain embodiments one R⁴ is halogen.

In certain embodiments a R⁴ is alkyl optionally substituted with 1, 2, 3, or 4 substituents selected from R²⁴.

In certain embodiments one R⁴ is alkyl optionally substituted with 1, 2, 3, or 4 substituents selected from R²⁴.

In certain embodiments a R⁴ is haloalkyl optionally substituted with 1, 2, 3, or 4 substituents selected from R²⁴.

In certain embodiments one R⁴ is haloalkyl optionally substituted with 1, 2, 3, or 4 substituents selected from R²⁴.

In certain embodiments a R⁴ is alkenyl optionally substituted with 1, 2, 3, or 4 substituents selected from R²⁴.

In certain embodiments one R⁴ is alkenyl optionally substituted with 1, 2, 3, or 4 substituents selected from R²⁴.

In certain embodiments a R⁴ is alkynyl optionally substituted with 1, 2, 3, or 4 substituents selected from R²⁴.

In certain embodiments one R⁴ is alkynyl optionally substituted with 1, 2, 3, or 4 substituents selected from R²⁴.

In certain embodiments a R⁴ is heterocycle optionally substituted with 1, 2, 3, or 4 substituents selected from R²⁴.

In certain embodiments one R⁴ is heterocycle optionally substituted with 1, 2, 3, or 4 substituents selected from R²⁴.

In certain embodiments a R⁴ is aryl optionally substituted with 1, 2, 3, or 4 substituents selected from R²⁴.

In certain embodiments one R⁴ is aryl optionally substituted with 1, 2, 3, or 4 substituents selected from R²⁴.

In certain embodiments a R⁴ is heteroaryl optionally substituted with 1, 2, 3, or 4 substituents selected from R²⁴.

In certain embodiments one R⁴ is heteroaryl optionally substituted with 1, 2, 3, or 4 substituents selected from R²⁴.

In certain embodiments a R⁴ is cyano.

In certain embodiments one R⁴ is cyano.

In certain embodiments a R⁴ is nitro.

In certain embodiments one R⁴ is nitro.

In certain embodiments a R⁴ is —C(O)R¹⁰.

In certain embodiments one R⁴ is —C(O)R¹⁰.

In certain embodiments a R⁴ is —OC(O)R¹⁰.

In certain embodiments one R⁴ is —OC(O)R¹⁰.

In certain embodiments a R⁴ is —NR¹¹C(O)R¹⁰.

In certain embodiments one R⁴ is —NR¹¹C(O)R¹⁰.

In certain embodiments a R⁴ is —OR¹¹.

In certain embodiments one R⁴ is —OR¹¹.

In certain embodiments a R⁴ is —NR¹¹R¹².

In certain embodiments one R⁴ is —NR¹¹R¹².

In certain embodiments a R⁴ is —S(O)R¹⁰.

In certain embodiments one R⁴ is —S(O)R¹⁰.

In certain embodiments a R⁴ is —S(O)₂R¹⁰.

In certain embodiments one R⁴ is —S(O)₂R¹⁰.

In certain embodiments a R⁴ is —OS(O)R¹⁰.

In certain embodiments one R⁴ is —OS(O)R¹⁰.

In certain embodiments a R⁴ is —OS(O)₂R¹⁰.

In certain embodiments one R⁴ is —OS(O)₂R¹⁰.

In certain embodiments a R⁴ is —NR¹¹S(O)R¹⁰.

In certain embodiments one R⁴ is —NR¹¹S(O)R¹⁰.

In certain embodiments a R⁴ is —NR¹¹S(O)₂R¹⁰.

In certain embodiments one R⁴ is —NR¹¹S(O)₂R¹⁰.

In certain embodiments a R⁴ is —SR¹¹.

In certain embodiments one R⁴ is —SR¹¹.

### Embodiments of R⁵

In certain embodiments a R⁵ is hydrogen.

In certain embodiments one R⁵ is hydrogen.

In certain embodiments all R⁵ groups are hydrogen.

In certain embodiments a R⁵ is halogen.

In certain embodiments one R⁵ is halogen.

In certain embodiments a R⁵ is alkyl optionally substituted with 1, 2, 3, or 4 substituents selected from R²⁵.

In certain embodiments one R⁵ is alkyl optionally substituted with 1, 2, 3, or 4 substituents selected from R²⁵.

In certain embodiments a R⁵ is haloalkyl optionally substituted with 1, 2, 3, or 4 substituents selected from R²⁵.

In certain embodiments one R⁵ is haloalkyl optionally substituted with 1, 2, 3, or 4 substituents selected from R²⁵.

In certain embodiments a R⁵ is alkenyl optionally substituted with 1, 2, 3, or 4 substituents selected from R²⁵.

In certain embodiments one R⁵ is alkenyl optionally substituted with 1, 2, 3, or 4 substituents selected from R²⁵.

In certain embodiments a R⁵ is alkynyl optionally substituted with 1, 2, 3, or 4 substituents selected from R²⁵.

In certain embodiments one R⁵ is alkynyl optionally substituted with 1, 2, 3, or 4 substituents selected from R²⁵.

In certain embodiments a R⁵ is heterocycle optionally substituted with 1, 2, 3, or 4 substituents selected from R²⁵.

In certain embodiments one R⁵ is heterocycle optionally substituted with 1, 2, 3, or 4 substituents selected from R²⁵.

In certain embodiments a R⁵ is aryl optionally substituted with 1, 2, 3, or 4 substituents selected from R²⁵.

In certain embodiments one R⁵ is aryl optionally substituted with 1, 2, 3, or 4 substituents selected from R²⁵.

In certain embodiments a R⁵ is heteroaryl optionally substituted with 1, 2, 3, or 4 substituents selected from R²⁵.

In certain embodiments one R⁵ is heteroaryl optionally substituted with 1, 2, 3, or 4 substituents selected from R²⁵.

In certain embodiments a R⁵ is cyano.

In certain embodiments one R⁵ is cyano.

In certain embodiments a R⁵ is nitro.

In certain embodiments one R⁵ is nitro.

In certain embodiments a R⁵ is —C(O)R¹⁰.

In certain embodiments one R⁵ is —C(O)R¹⁰.

In certain embodiments a R⁵ is —OC(O)R¹⁰.

In certain embodiments one R⁵ is —OC(O)R¹⁰.

In certain embodiments a R⁵ is —NR¹¹C(O)R¹⁰.

In certain embodiments one R⁵ is —NR¹¹C(O)R¹⁰.

In certain embodiments a R⁵ is —OR¹¹.

In certain embodiments one R⁵ is —OR¹¹.

In certain embodiments a R⁵ is —NR¹¹R¹².

In certain embodiments one R⁵ is —NR¹¹R¹².

In certain embodiments a R⁵ is —S(O)R¹⁰.

In certain embodiments one R⁵ is —S(O)R¹⁰.

In certain embodiments a R⁵ is —S(O)₂R¹⁰.

In certain embodiments one R⁵ is —S(O)₂R¹⁰.

In certain embodiments a R⁵ is —OS(O)R¹⁰.

In certain embodiments one R⁵ is —OS(O)R¹⁰.

In certain embodiments a R⁵ is —OS(O)₂R¹⁰.

In certain embodiments one R⁵ is —OS(O)₂R¹⁰.

In certain embodiments a R⁵ is —NR¹¹S(O)R¹⁰.

In certain embodiments one R⁵ is —NR¹¹S(O)R¹⁰.

In certain embodiments a R⁵ is —NR¹¹S(O)₂R¹⁰.

In certain embodiments one R⁵ is —NR¹¹S(O)₂R¹⁰.

In certain embodiments a R⁵ is —SR¹¹.

In certain embodiments one R⁵ is —SR¹¹.

### Embodiments of R⁶

In certain embodiments a R⁶ is hydrogen.

In certain embodiments one R⁶ is hydrogen.

In certain embodiments all R⁶ groups are hydrogen.

In certain embodiments a R⁶ is halogen.

In certain embodiments one R⁶ is halogen.

In certain embodiments a R⁶ is alkyl optionally substituted with 1, 2, 3, or 4 substituents selected from R²⁶.

In certain embodiments one R⁶ is alkyl optionally substituted with 1, 2, 3, or 4 substituents selected from R²⁶.

In certain embodiments a R⁶ is haloalkyl optionally substituted with 1, 2, 3, or 4 substituents selected from R²⁶.

In certain embodiments one R⁶ is haloalkyl optionally substituted with 1, 2, 3, or 4 substituents selected from R²⁶.

In certain embodiments a R⁶ is alkenyl optionally substituted with 1, 2, 3, or 4 substituents selected from R²⁶.

In certain embodiments one R⁶ is alkenyl optionally substituted with 1, 2, 3, or 4 substituents selected from R²⁶.

In certain embodiments a R⁶ is alkynyl optionally substituted with 1, 2, 3, or 4 substituents selected from R²⁶.

In certain embodiments one R⁶ is alkynyl optionally substituted with 1, 2, 3, or 4 substituents selected from R²⁶.

In certain embodiments a R⁶ is heterocycle optionally substituted with 1, 2, 3, or 4 substituents selected from R²⁶.

In certain embodiments one R⁶ is heterocycle optionally substituted with 1, 2, 3, or 4 substituents selected from R²⁶.

In certain embodiments a R⁶ is aryl optionally substituted with 1, 2, 3, or 4 substituents selected from R²⁶.

In certain embodiments one R⁶ is aryl optionally substituted with 1, 2, 3, or 4 substituents selected from R²⁶.

In certain embodiments a R⁶ is heteroaryl optionally substituted with 1, 2, 3, or 4 substituents selected from R²⁶.

In certain embodiments one R⁶ is heteroaryl optionally substituted with 1, 2, 3, or 4 substituents selected from R²⁶.

In certain embodiments a R⁶ is cyano.

In certain embodiments one R⁶ is cyano.

In certain embodiments a R⁶ is nitro.

In certain embodiments one R⁶ is nitro.

In certain embodiments a R⁶ is —C(O)R¹⁰.

In certain embodiments one R⁶ is —C(O)R¹⁰.

In certain embodiments a R⁶ is —OC(O)R¹⁰.

In certain embodiments one R⁶ is —OC(O)R¹⁰.

In certain embodiments a R⁶ is —NR¹¹C(O)R¹⁰.

In certain embodiments one R⁶ is —NR¹¹C(O)R¹⁰.

In certain embodiments a R⁶ is —OR¹¹.

In certain embodiments one R⁶ is —OR¹¹.

In certain embodiments a R⁶ is —NR¹¹R¹².

In certain embodiments one R⁶ is —NR¹¹R¹².

In certain embodiments a R⁶ is —S(O)R¹⁰.

In certain embodiments one R⁶ is —S(O)R¹⁰.

In certain embodiments a R⁶ is —S(O)₂R¹⁰.

In certain embodiments one R⁶ is —S(O)₂R¹⁰.

In certain embodiments a R⁶ is —OS(O)R¹⁰.

In certain embodiments one R⁶ is —OS(O)R¹⁰.

In certain embodiments a R⁶ is —OS(O)₂R¹⁰.

In certain embodiments one R⁶ is —OS(O)₂R¹⁰.

In certain embodiments a R⁶ is —NR¹¹S(O)R¹⁰.

In certain embodiments one R⁶ is —NR¹¹S(O)R¹⁰.

In certain embodiments a R⁶ is —NR¹¹S(O)₂R¹⁰.

In certain embodiments one R⁶ is —NR¹¹S(O)₂R¹⁰.

In certain embodiments a R⁶ is —SR¹¹.

In certain embodiments one R⁶ is —SR¹¹.

### Embodiments of R¹⁰

In certain embodiments R¹⁰ is independently selected at each instance from hydrogen, and alkyl.

In certain embodiments each R¹⁰ is hydrogen.

In certain embodiments each R¹⁰ is alkyl.

In certain embodiments each R¹⁰ is methyl.

In certain embodiments a R¹⁰ is alkyl optionally substituted as allowed by valence with 1, 2, 3, or 4 substituents selected from R³¹.

In certain embodiments a R¹⁰ is haloalkyl optionally substituted as allowed by valence with 1, 2, 3, or 4 substituents selected from R³¹.

In certain embodiments a R¹⁰ is alkenyl or alkynyl optionally substituted as allowed by valence with 1, 2, 3, or 4 substituents selected from R³¹.

In certain embodiments a R¹⁰ is —OR¹¹.

In certain embodiments a R¹⁰ is —NR¹¹R¹².

In certain embodiments a R¹⁰ is —SR¹¹.

In certain embodiments a R¹⁰ is aryl optionally substituted as allowed by valence with 1, 2, 3, or 4 substituents selected from R³⁰.

In certain embodiments a R¹⁰ is phenyl optionally substituted as allowed by valence with 1, 2, 3, or 4 substituents selected from R³⁰.

In certain embodiments a R¹⁰ is heterocycle optionally substituted as allowed by valence with 1, 2, 3, or 4 substituents selected from R³⁰.

In certain embodiments a R¹⁰ is heteroaryl optionally substituted as allowed by valence with 1, 2, 3, or 4 substituents selected from R³⁰.

Embodiments of R¹¹ and R¹²

In certain embodiments R¹¹ and R¹² are hydrogen.

In certain embodiments a R¹¹ is hydrogen.

In certain embodiments a R¹² is hydrogen.

In certain embodiments R¹¹ and R¹² are alkyl.

In certain embodiments a R¹¹ is alkyl.

In certain embodiments a R¹² is alkyl.

In certain embodiments R¹¹ and R¹² are methyl.

In certain embodiments a R¹¹ is methyl.

In certain embodiments a R¹² is methyl.

In certain embodiments R¹¹ or R¹² is haloalkyl optionally substituted as allowed by valence with 1, 2, 3, or 4 substituents selected from R³¹.

In certain embodiments R¹¹ or R¹² is alkenyl or alkynyl optionally substituted as allowed by valence with 1, 2, 3, or 4 substituents selected from R³¹.

In certain embodiments R¹¹ or R¹² is aryl optionally substituted as allowed by valence with 1, 2, 3, or 4 substituents selected from R³¹.

In certain embodiments R¹¹ or R¹² is phenyl optionally substituted as allowed by valence with 1, 2, 3, or 4 substituents selected from R³¹.

In certain embodiments R¹¹ or R¹² is heterocycle optionally substituted as allowed by valence with 1, 2, 3, or 4 substituents selected from R³¹.

In certain embodiments R¹¹ or R¹² is heteroaryl optionally substituted as allowed by valence with 1, 2, 3, or 4 substituents selected from R³¹.

In certain embodiments R¹¹ or R¹² is —C(O)R⁴⁰ optionally substituted as allowed by valence with 1, 2, 3, or 4 substituents selected from R³¹.

In certain embodiments R¹¹ or R¹² is —S(O)R⁴⁰ optionally substituted as allowed by valence with 1, 2, 3, or 4 substituents selected from R³¹.

In certain embodiments R¹¹ or R¹² is —S(O)₂R⁴⁰ optionally substituted as allowed by valence with 1, 2, 3, or 4 substituents selected from R³¹.

Embodiments of R²¹, R²², R²³, R²⁴, R²⁵, and R²⁶

In certain embodiments R²¹, R²², R²³, R²⁴, R²⁵, and R²⁶ are selected at each instance from hydrogen, halogen, alkyl, and haloalkyl.

In certain embodiments at least one of R²¹, R²², R²³, R²⁴, R²⁵, and R²⁶ is halogen.

In certain embodiments at least one of R²¹, R²², R²³, R²⁴, R²⁵, and R²⁶ is alkyl optionally substituted as allowed by valence with 1, 2, 3, or 4 substituents selected from R⁴³.

In certain embodiments at least one of R²¹, R²², R²³, R²⁴, R²⁵, and R²⁶ is haloalkyl optionally substituted as allowed by valence with 1, 2, 3, or 4 substituents selected from R⁴³.

In certain embodiments at least one of R²¹, R²², R²³, R²⁴, R²⁵, and R²⁶ is alkenyl optionally substituted as allowed by valence with 1, 2, 3, or 4 substituents selected from R⁴³.

In certain embodiments at least one of R²¹, R²², R²³, R²⁴, R²⁵, and R²⁶ is alkynyl optionally substituted as allowed by valence with 1, 2, 3, or 4 substituents selected from R⁴³.

In certain embodiments at least one of R²¹, R²², R²³, R²⁴, R²⁵, and R²⁶ is heterocycle optionally substituted as allowed by valence with 1, 2, 3, or 4 substituents selected from R⁴³.

In certain embodiments at least one of R²¹, R²², R²³, R²⁴, R²⁵, and R²⁶ is aryl optionally substituted as allowed by valence with 1, 2, 3, or 4 substituents selected from R⁴³.

In certain embodiments at least one of R²¹, R²², R²³, R²⁴, R²⁵, and R²⁶ is heteroaryl optionally substituted as allowed by valence with 1, 2, 3, or 4 substituents selected from R⁴³.

In certain embodiments at least one of R²¹, R²², R²³, R²⁴, R²⁵, and R²⁶ is cyano.

In certain embodiments at least one of R²¹, R²², R²³, R²⁴, R²⁵, and R²⁶ is nitro.

In certain embodiments at least one of R²¹, R²², R²³, R²⁴, R²⁵, and R²⁶ is —C(O)R⁴⁰.

In certain embodiments at least one of R²¹, R²², R²³, R²⁴, R²⁵, and R²⁶ is —OC(O)R⁴⁰.

In certain embodiments at least one of R²¹, R²², R²³, R²⁴, R²⁵, and R²⁶ is —NR⁴¹C(O)R⁴⁰.

In certain embodiments at least one of R²¹, R²², R²³, R²⁴, R²⁵, and R²⁶ is —OR⁴¹.

In certain embodiments at least one of R²¹, R²², R²³, R²⁴, R²⁵, and R²⁶ is —NR⁴¹R⁴².

In certain embodiments at least one of R²¹, R²², R²³, R²⁴, R²⁵, and R²⁶ is —S(O)R⁴⁰.

In certain embodiments at least one of R²¹, R²², R²³, R²⁴, R²⁵, and R²⁶ is —OS(O)R⁴⁰.

In certain embodiments at least one of R²¹, R²², R²³, R²⁴, R²⁵, and R²⁶ is —OS(O)₂R⁴⁰.

In certain embodiments at least one of R²¹, R²², R²³, R²⁴, R²⁵, and R²⁶ is —NR⁴¹S(O)R⁴⁰.

In certain embodiments at least one of R²¹, R²², R²³, R²⁴, R²⁵, and R²⁶ is —NR⁴¹S(O)₂R⁴⁰.

In certain embodiments at least one of R²¹, R²², R²³, R²⁴, R²⁵, and R²⁶ is —SR⁴¹.

Embodiments of R³⁰ and R³¹

In certain embodiments R³⁰ or R³¹ is hydrogen.

In certain embodiments R³⁰ or R³¹ is halogen.

In certain embodiments R³⁰ or R³¹ is alkyl optionally substituted as allowed by valence with 1, 2, 3, or 4 substituents selected from R⁴³.

In certain embodiments R³⁰ or R³¹ is haloalkyl optionally substituted as allowed by valence with 1, 2, 3, or 4 substituents selected from R⁴³.

In certain embodiments R³⁰ or R³¹ is alkenyl optionally substituted as allowed by valence with 1, 2, 3, or 4 substituents selected from R⁴³.

In certain embodiments R³⁰ or R³¹ is alkynyl optionally substituted as allowed by valence with 1, 2, 3, or 4 substituents selected from R⁴³.

In certain embodiments R³⁰ or R³¹ is heterocycle optionally substituted as allowed by valence with 1, 2, 3, or 4 substituents selected from R⁴³.

In certain embodiments R³⁰ or R³¹ is aryl optionally substituted as allowed by valence with 1, 2, 3, or 4 substituents selected from R⁴³.

In certain embodiments R³⁰ or R³¹ is heteroaryl optionally substituted as allowed by valence with 1, 2, 3, or 4 substituents selected from R⁴³.

In certain embodiments R³⁰ or R³¹ is cyano.

In certain embodiments R³⁰ or R³¹ is nitro.

In certain embodiments R³⁰ or R³¹ is —C(O)R⁴⁰.

In certain embodiments R³⁰ or R³¹ is —OC(O)R⁴⁰.

In certain embodiments R³⁰ or R³¹ is —NR⁴¹C(O)R⁴⁰.

In certain embodiments R³⁰ or R³¹ is —OR⁴¹.

In certain embodiments R³⁰ or R³¹ is —NR⁴¹R⁴².

In certain embodiments R³⁰ or R³¹ is —S(O)R⁴⁰.

In certain embodiments R³⁰ or R³¹ is —S(O)₂R⁴⁰.

In certain embodiments R³⁰ or R³¹ is —OS(O)R⁴⁰.

In certain embodiments R³⁰ or R³¹ is —OS(O)₂R⁴⁰.

In certain embodiments R³⁰ or R³¹ is —NR⁴¹S(O)R⁴⁰.

In certain embodiments R³⁰ or R³¹ is —NR⁴¹S(O)₂R⁴⁰.

In certain embodiments R³⁰ or R³¹ is —SR⁴¹.

### Embodiments of R⁴

In certain embodiments a R⁴⁰ is hydrogen.

In certain embodiments a R⁴⁰ is alkyl optionally substituted as allowed by valence with 1, 2, 3, or 4 substituents selected from R⁴³.

In certain embodiments a R⁴⁰ is haloalkyl optionally substituted as allowed by valence with 1, 2, 3, or 4 substituents selected from R⁴³.

In certain embodiments a R⁴⁰ is alkenyl optionally substituted as allowed by valence with 1, 2, 3, or 4 substituents selected from R⁴³.

In certain embodiments a R⁴⁰ is alkynyl optionally substituted as allowed by valence with 1, 2, 3, or 4 substituents selected from R⁴³.

In certain embodiments a R⁴⁰ is aryl optionally substituted as allowed by valence with 1, 2, 3, or 4 substituents selected from R⁴³.

In certain embodiments a R⁴⁰ is heterocycle optionally substituted as allowed by valence with 1, 2, 3, or 4 substituents selected from R⁴³.

In certain embodiments a R⁴⁰ is heteroaryl optionally substituted as allowed by valence with 1, 2, 3, or 4 substituents selected from R⁴³.

In certain embodiments a R⁴⁰ is amino optionally substituted as allowed by valence with 1, 2, 3, or 4 substituents selected from R⁴³.

In certain embodiments a R⁴⁰ is hydroxyl optionally substituted as allowed by valence with 1, 2, 3, or 4 substituents selected from R⁴³.

In certain embodiments a R⁴⁰ is alkoxy optionally substituted as allowed by valence with 1, 2, 3, or 4 substituents selected from R⁴³.

In certain embodiments a R⁴⁰ is heteroaryl optionally substituted as allowed by valence with 1, 2, 3, or 4 substituents selected from R⁴³.

Embodiments of R⁴ and R⁴²

In certain embodiments R⁴¹ and R⁴² are hydrogen.

In certain embodiments a R⁴¹ is hydrogen.

In certain embodiments a R⁴² is hydrogen.

In certain embodiments R⁴¹ and R⁴² are alkyl.

In certain embodiments a R⁴¹ is alkyl.

In certain embodiments a R⁴² is alkyl.

In certain embodiments R⁴¹ and R⁴² are methyl.

In certain embodiments a R⁴¹ is methyl.

In certain embodiments a R⁴² is methyl.

In certain embodiments R⁴¹ or R⁴² is haloalkyl optionally substituted as allowed by valence with 1, 2, 3, or 4 substituents selected from R⁴³.

In certain embodiments R⁴¹ or R⁴² is alkenyl or alkynyl optionally substituted as allowed by valence with 1, 2, 3, or 4 substituents selected from R⁴³.

In certain embodiments R⁴¹ or R⁴² is aryl optionally substituted as allowed by valence with 1, 2, 3, or 4 substituents selected from R⁴³.

In certain embodiments R⁴¹ or R⁴² is phenyl optionally substituted as allowed by valence with 1, 2, 3, or 4 substituents selected from R⁴³.

In certain embodiments R⁴¹ or R⁴² is heterocycle optionally substituted as allowed by valence with 1, 2, 3, or 4 substituents selected from R⁴³.

In certain embodiments R⁴¹ or R⁴² is heteroaryl optionally substituted as allowed by valence with 1, 2, 3, or 4 substituents selected from R⁴³.

In certain embodiments R⁴¹ or R⁴² is —C(O)R⁴⁰ optionally substituted as allowed by valence with 1, 2, 3, or 4 substituents selected from R⁴³.

In certain embodiments R⁴¹ or R⁴² is —S(O)R⁴⁰ optionally substituted as allowed by valence with 1, 2, 3, or 4 substituents selected from R⁴³.

In certain embodiments R⁴¹ or R⁴² is —S(O)₂R⁴⁰ optionally substituted as allowed by valence with 1, 2, 3, or 4 substituents selected from R⁴³.

### Embodiments of R⁴³

In certain embodiments a R⁴³ is halogen.

In certain embodiments a R⁴³ is cyano.

In certain embodiments a R⁴³ is nitro.

In certain embodiments a R⁴³ is alkyl.

In certain embodiments a R⁴³ is haloalkyl.

In certain embodiments a R⁴³ is alkenyl.

In certain embodiments a R⁴³ is alkynyl.

In certain embodiments a R⁴³ is aryl.

In certain embodiments a R⁴³ is heterocycle.

In certain embodiments a R⁴³ is heteroaryl.

In certain embodiments a R43 is amino.

In certain embodiments a R⁴³ is hydroxyl.

In certain embodiments a R⁴³ is alkoxy.

In certain embodiments a R⁴³ is —NHalkyl.

In certain embodiments a R⁴³ is —N(alkyl)₂.

In certain embodiments a R⁴³ is —OC(O)alkyl.

In certain embodiments a R⁴³ is —NHC(O)alkyl.

In certain embodiments a R⁴³ is —N(alkyl)C(O)alkyl.

### Embodiments of R¹⁰¹

In certain embodiments a R¹⁰¹ is halogen.

In certain embodiments a R¹⁰¹ is F.

In certain embodiments a R¹⁰¹ is Cl.

In certain embodiments a R¹⁰¹ is Br.

In certain embodiments a R¹⁰¹ is alkyl.

In certain embodiments a R¹⁰¹ is methyl.

In certain embodiments a R¹⁰¹ is alkyl optionally substituted as allowed by valence with 1, 2, 3, or 4 substituents selected from R²¹.

In certain embodiments a R¹⁰¹ is haloalkyl optionally substituted as allowed by valence with 1, 2, 3, or 4 substituents selected from R²¹.

In certain embodiments a R¹⁰¹ is alkenyl optionally substituted as allowed by valence with 1, 2, 3, or 4 substituents selected from R²¹.

In certain embodiments a R¹⁰¹ is alkynyl optionally substituted as allowed by valence with 1, 2, 3, or 4 substituents selected from R²¹.

In certain embodiments a R¹⁰¹ is heterocycle optionally substituted as allowed by valence with 1, 2, 3, or 4 substituents selected from R²¹.

In certain embodiments a R¹⁰¹ is aryl optionally substituted as allowed by valence with 1, 2, 3, or 4 substituents selected from R²¹.

In certain embodiments a R¹⁰¹ is heteroaryl optionally substituted as allowed by valence with 1, 2, 3, or 4 substituents selected from R²¹.

In certain embodiments a R¹⁰¹ is cyano.

In certain embodiments a R¹⁰¹ is nitro.

In certain embodiments a R¹⁰¹ is —C(O)R¹⁰.

In certain embodiments a R¹⁰¹ is —OC(O)R¹⁰.

In certain embodiments a R¹⁰¹ is —NR¹¹C(O)R¹⁰.

In certain embodiments a R¹⁰¹ is —OR¹¹.

In certain embodiments a R¹⁰¹ is —NR¹¹R¹².

In certain embodiments a R¹⁰¹ is —S(O)R¹⁰.

In certain embodiments a R¹⁰¹ is —S(O)₂R¹⁰.

In certain embodiments a R¹⁰¹ is —OS(O)R¹⁰.

In certain embodiments a R¹⁰¹ is —OS(O)₂R¹⁰.

In certain embodiments a R¹⁰¹ is —NR¹¹S(O)R¹⁰.

In certain embodiments a R¹⁰¹ is —NR¹¹S(O)₂R¹⁰.

In certain embodiments a R¹⁰¹ is —SR¹¹.

### Embodiments of R¹⁰²

In certain embodiments a R¹⁰² is halogen.

In certain embodiments a R¹⁰² is F.

In certain embodiments a R¹⁰² is Cl.

In certain embodiments a R¹⁰² is Br.

In certain embodiments a R¹⁰² is alkyl.

In certain embodiments a R¹⁰² is methyl.

In certain embodiments a R¹⁰² is alkyl optionally substituted as allowed by valence with 1, 2, 3, or 4 substituents selected from R²¹.

In certain embodiments a R¹⁰² is haloalkyl optionally substituted as allowed by valence with 1, 2, 3, or 4 substituents selected from R²¹.

In certain embodiments a R¹⁰² is alkenyl optionally substituted as allowed by valence with 1, 2, 3, or 4 substituents selected from R²¹.

In certain embodiments a R¹⁰² is alkynyl optionally substituted as allowed by valence with 1, 2, 3, or 4 substituents selected from R²¹.

In certain embodiments a R¹⁰² is heterocycle optionally substituted as allowed by valence with 1, 2, 3, or 4 substituents selected from R²¹.

In certain embodiments a R¹⁰² is aryl optionally substituted as allowed by valence with 1, 2, 3, or 4 substituents selected from R²¹.

In certain embodiments a R¹⁰² is heteroaryl optionally substituted as allowed by valence with 1, 2, 3, or 4 substituents selected from R²¹.

In certain embodiments a R¹⁰² is cyano.

In certain embodiments a R¹⁰² is nitro.

In certain embodiments a R¹⁰² is —C(O)R¹⁰.

In certain embodiments a R¹⁰² is —OC(O)R¹⁰.

In certain embodiments a R¹⁰² is —NR¹¹C(O)R¹⁰.

In certain embodiments a R¹⁰² is —OR¹¹.

In certain embodiments a R¹⁰² is —NR¹¹R¹².

In certain embodiments a R¹⁰² is —S(O)R¹⁰.

In certain embodiments a R¹⁰² is —S(O)₂R¹⁰.

In certain embodiments a R¹⁰² is —OS(O)R¹⁰.

In certain embodiments a R¹⁰² is —OS(O)₂R¹⁰.

In certain embodiments a R¹⁰² is —NR¹¹S(O)R¹⁰.

In certain embodiments a R¹⁰² is —NR¹¹S(O)₂R¹⁰.

In certain embodiments a R¹⁰² is —SR¹¹.

### Embodiments of R²⁰⁰

In certain embodiments a R²⁰⁰ is halogen.

In certain embodiments a R²⁰⁰ is F.

In certain embodiments a R²⁰⁰ is Cl.

In certain embodiments a R²⁰⁰ is Br.

In certain embodiments a R²⁰⁰ is alkyl.

In certain embodiments a R²⁰⁰ is methyl.

In certain embodiments a R²⁰⁰ is alkyl optionally substituted as allowed by valence with 1, 2, 3, or 4 substituents selected from R²¹.

In certain embodiments a R²⁰⁰ is haloalkyl optionally substituted as allowed by valence with 1, 2, 3, or 4 substituents selected from R²¹.

In certain embodiments a R²⁰⁰ is alkenyl optionally substituted as allowed by valence with 1, 2, 3, or 4 substituents selected from R²¹.

In certain embodiments a R²⁰⁰ is alkynyl optionally substituted as allowed by valence with 1, 2, 3, or 4 substituents selected from R²¹.

In certain embodiments a R²⁰⁰ is heterocycle optionally substituted as allowed by valence with 1, 2, 3, or 4 substituents selected from R²¹.

In certain embodiments a R²⁰⁰ is aryl optionally substituted as allowed by valence with 1, 2, 3, or 4 substituents selected from R²¹.

In certain embodiments a R²⁰⁰ is heteroaryl optionally substituted as allowed by valence with 1, 2, 3, or 4 substituents selected from R²¹.

In certain embodiments a R²⁰⁰ is cyano.

In certain embodiments a R²⁰⁰ is nitro.

In certain embodiments a R²⁰⁰ is —C(O)R¹⁰.

In certain embodiments a R²⁰⁰ is —OC(O)R¹⁰.

In certain embodiments a R²⁰⁰ is —NR¹¹C(O)R¹⁰.

In certain embodiments a R²⁰⁰ is —OR¹¹.

In certain embodiments a R²⁰⁰ is —NR¹¹R¹².

In certain embodiments a R²⁰⁰ is —S(O)R¹⁰.

In certain embodiments a R²⁰⁰ is —S(O)₂R¹⁰.

In certain embodiments a R²⁰⁰ is —OS(O)R¹⁰.

In certain embodiments a R²⁰⁰ is —OS(O)₂R¹⁰.

In certain embodiments a R²⁰⁰ is —NR¹¹S(O)R¹⁰.

In certain embodiments a R²⁰⁰ is —NR¹¹S(O)₂R¹⁰.

In certain embodiments a R²⁰⁰ is —SR¹¹.

### Embodiments of “Alkyl”

In certain embodiments “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

In an alternative embodiment the “alkynyl” group is optionally substituted.

### Embodiments of “Haloalkyl”

In certain embodiments “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

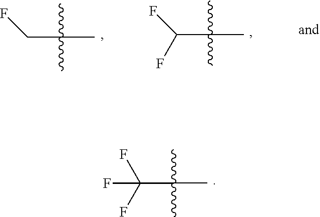

Additional non-limiting examples of “haloalkyl” include:

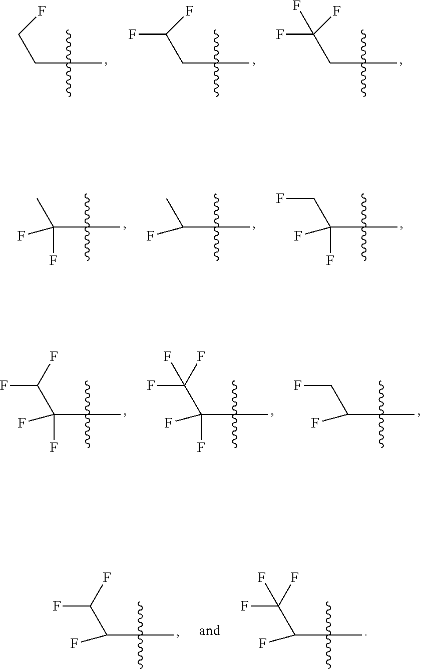

Additional non-limiting examples of “haloalkyl” include:

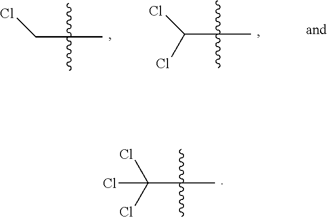

Additional non-limiting examples of “haloalkyl” include:

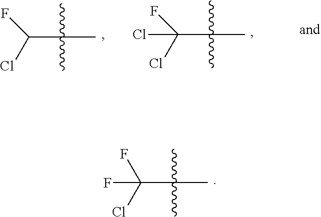

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

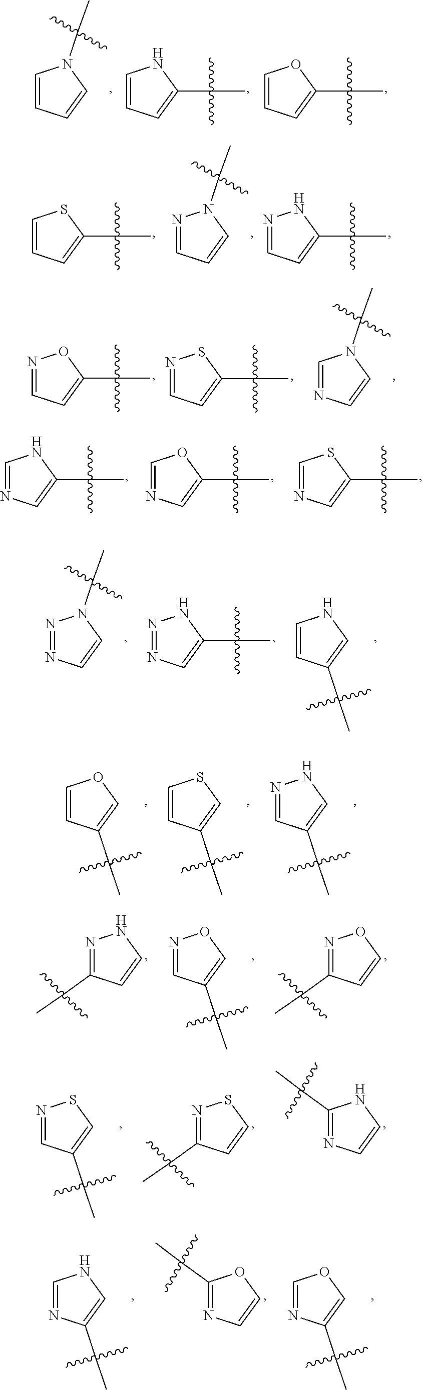

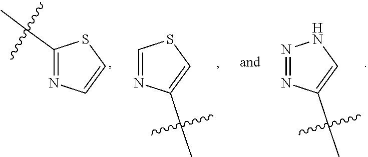

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

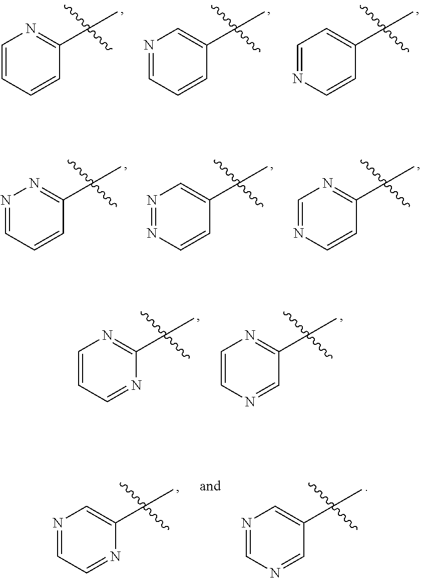

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

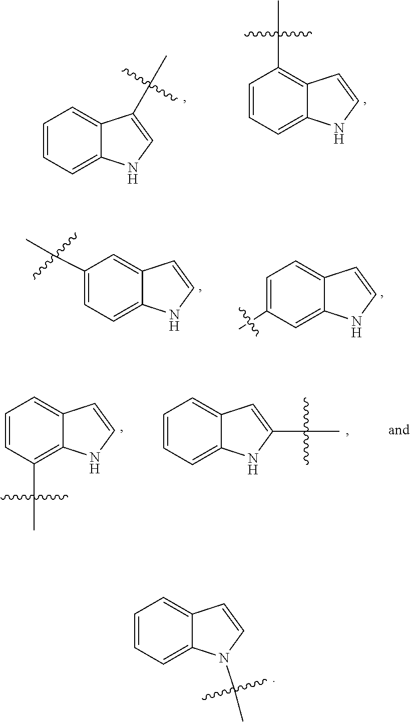

Additional non-limiting examples of “heteroaryl” groups that are bicyclic include:

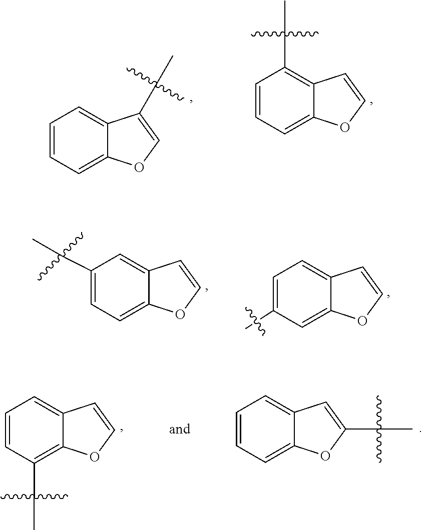

Additional non-limiting examples of “heteroaryl” groups that are bicyclic include:

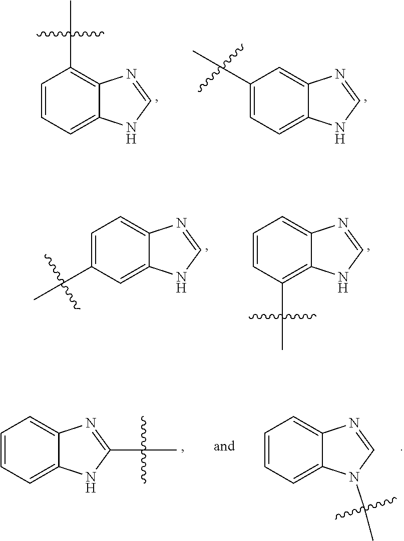 [SMILES: *c1cccc2[nH]cnc12 ; *c1ccc2[nH]cnc2c1 ; *c1ccc2nc[nH]c2c1 ; *c1cccc2nc[nH]c12 ; *c1nc2ccccc2[nH]1 ; *n1cnc2ccccc21]

In certain embodiments “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

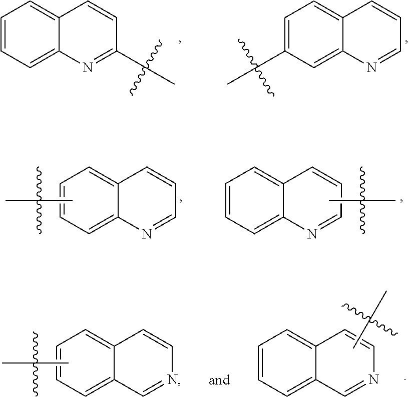

### Embodiments of “Heterocycle”

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

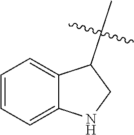 [SMILES: *C1CNc2ccccc21]

is a “heterocycle” group.

However,

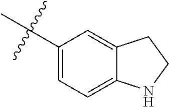 [SMILES: *c1ccc2c(c1)CCN2]

is an “aryl” group.

Non-limiting examples of “heterocycle” also include:

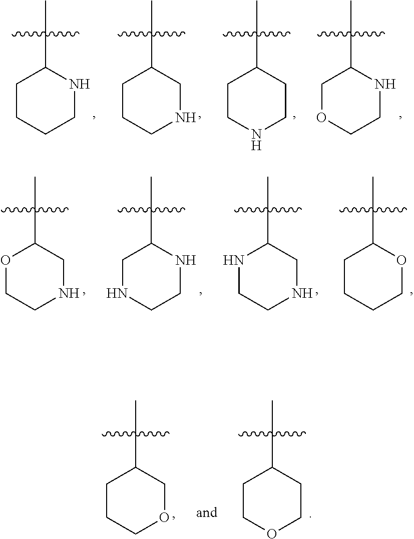

Additional non-limiting examples of “heterocycle” include:

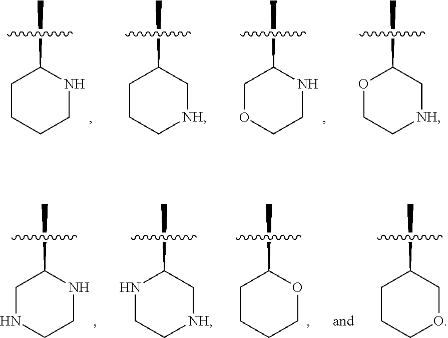

Additional non-limiting examples of “heterocycle” include:

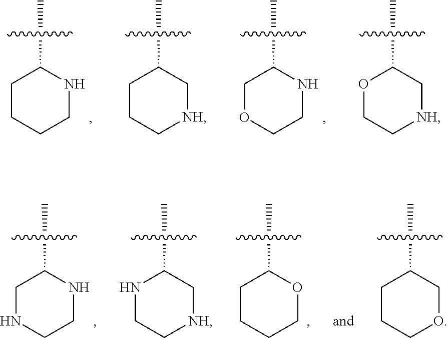

Non-limiting examples of “heterocycle” also include:

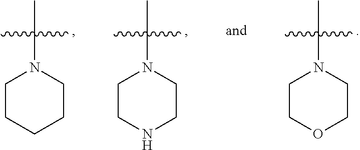 [SMILES: *N1CCCCC1 ; *N1CCNCC1 ; *N1CCOCC1]

Non-limiting examples of “heterocycle” also include:

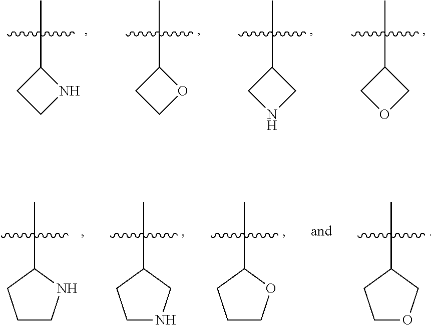

Non-limiting examples of “heterocycle” also include:

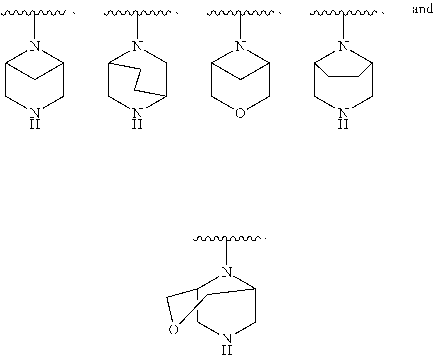

Non-limiting examples of “heterocycle” also include:

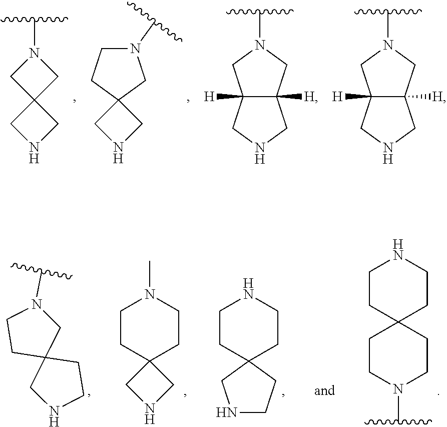

Non-limiting examples of “heterocycle” also include:

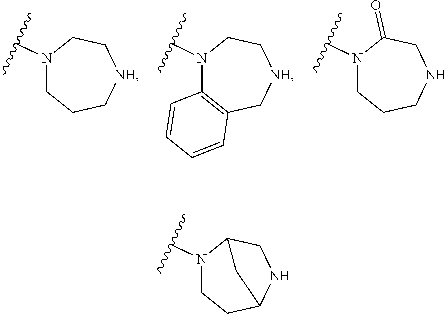 [SMILES: *N1CCCNCC1 ; *N1CCNCc2ccccc21 ; *N1CCCNCC1=O ; *N1CCC2CC1CN2]

Non-limiting examples of “heterocycle” also include:

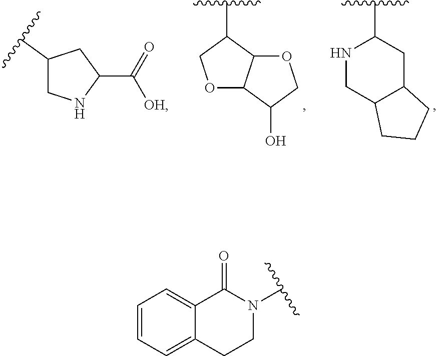 [SMILES: *C1CNC(C(=O)O)C1 ; *C1COC2C(O)COC12 ; *C1CC2CCCC2CN1 ; *N1CCc2ccccc2C1=O]

Non-limiting examples of “heterocycle” also include:

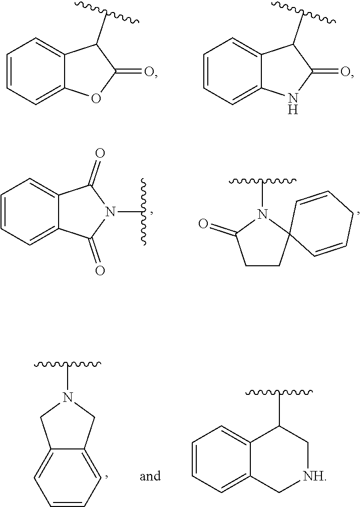

### Embodiments of “Aryl”

In certain embodiments “aryl” is a 6 carbon aromatic group (phenyl).

In certain embodiments “aryl” is a 10 carbon aromatic group (naphthyl).

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

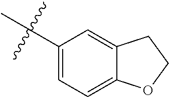 [SMILES: *c1ccc2c(c1)CCO2]

is an “aryl” group.

However,

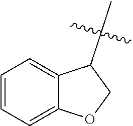 [SMILES: *C1COc2ccccc21]

is a “heterocycle” group.

### Embodiments of “Arylalkyl”

Non-limiting examples of “arylalkyl” include:

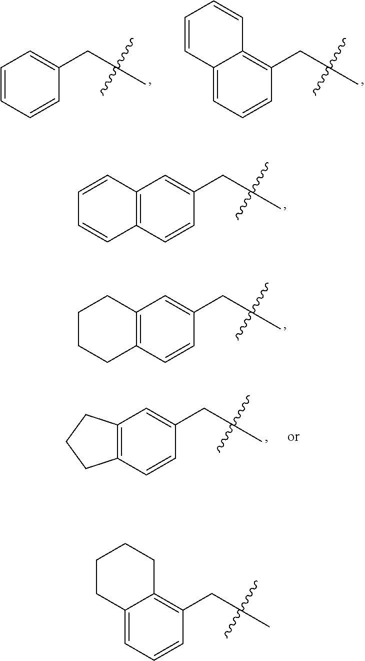 [SMILES: *Cc1ccccc1 ; *Cc1cccc2ccccc12 ; *Cc1ccc2ccccc2c1 ; *Cc1ccc2c(c1)CCCC2 ; *Cc1ccc2c(c1)CCC2 ; *Cc1cccc2c1CCCC2]

In certain embodiments “arylalkyl” is

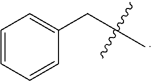 [SMILES: *Cc1ccccc1]

In certain embodiments the “arylalkyl” refers to a 2 carbon alkyl group substituted with an aryl group.

Non-limiting examples of “arylalkyl” include:

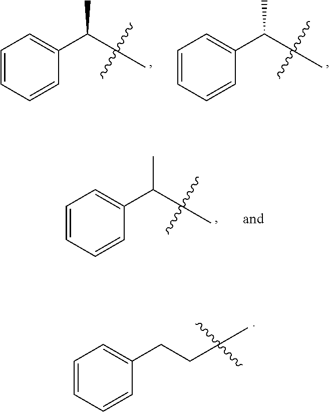

### Terminology

Compounds are described using standard nomenclature. Unless defined otherwise, all technical and scientific terms used herein have the same meaning as is commonly understood by one of skill in the art to which this invention belongs.

The protein stabilizing compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In certain embodiments the present invention includes protein stabilizing compounds with at least one desired isotopic substitution of an atom, at an amount above the natural abundance of the isotope, i.e., enriched. In certain embodiments the present invention includes protein stabilizing compounds that are not isotopically labeled.

Examples of isotopes that can be incorporated into protein stabilizing compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F, ³¹P, ³²P, ³⁵S, ³⁶Cl, and ¹²⁵I respectively.

In one embodiment, isotopically labelled protein stabilizing compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled protein stabilizing compound may be desirable for PET or SPECT studies. Isotopically labeled protein stabilizing compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.). In certain other embodiments, a variable group has a “′” or an “a” designation, which in one embodiment can be deuterated.

The protein stabilizing compound of the present invention may form a solvate with solvents (including water). Therefore, in one embodiment, the invention includes a solvated form of the active protein stabilizing compound. The term “solvate” refers to a molecular complex of a protein stabilizing compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a protein stabilizing compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C=O)NH₂ is attached through carbon of the keto (C=O) group.

The term “substituted”, as used herein, means that any one or more hydrogens on the designated atom or group is replaced with a moiety selected from the indicated group, provided that the designated atom's normal valence is not exceeded and the resulting protein stabilizing compound is stable. For example, when the substituent is oxo (i.e., =O) then two hydrogens on the atom are replaced. For example a pyridyl group substituted by oxo is a pyridone. Combinations of substituents and/or variables are permissible only if such combinations result in stable protein stabilizing compounds or useful synthetic intermediates.

“Alkyl” is a branched, straight chain, or cyclic saturated aliphatic hydrocarbon group. In one embodiment, the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₅alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such protein stabilizing compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

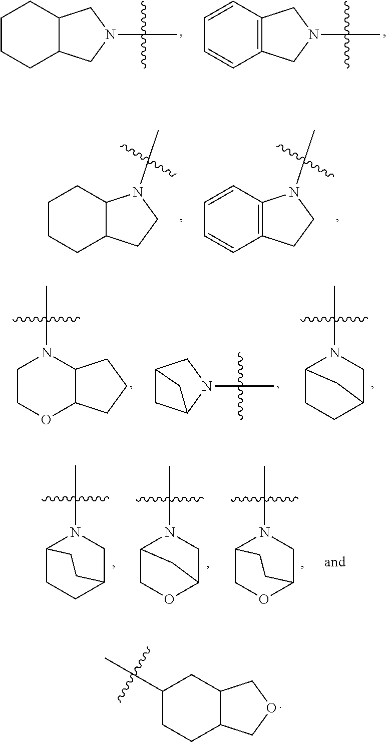

Unless otherwise drawn or clear from the context, the term “bicyclic heterocycle” includes cis and trans diastereomers. Non-limiting examples of chiral bicyclic heterocycles include:

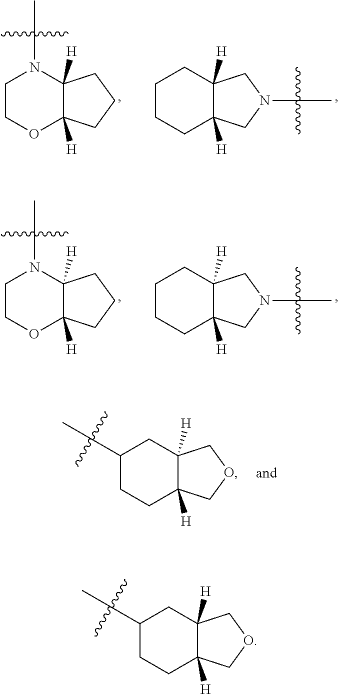

In certain alternative embodiments the term “heterocycle” refers to saturated and partially saturated heteroatom-containing ring radicals, where the heteroatoms may be selected from N, S, O, B, Si, and P.

The term “bicycle” refers to a ring system wherein two rings are fused together and each ring is independently selected from carbocycle, heterocycle, aryl, and heteroaryl. Non-limiting examples of bicycle groups include:

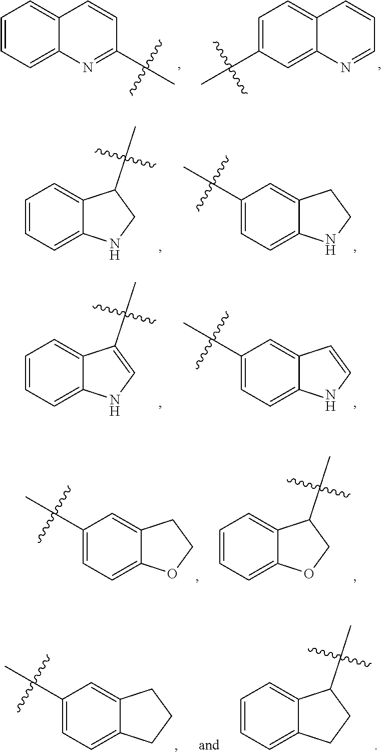

When the term “bicycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent bicycle groups include:

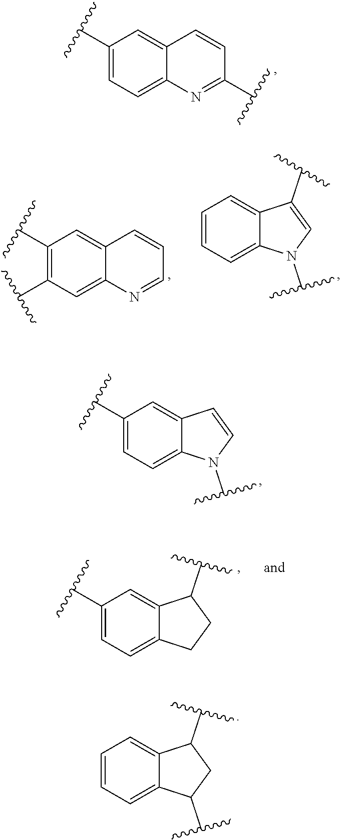

“Heteroaryl” refers to a stable monocyclic, bicyclic, or multicyclic aromatic ring which contains from 1 to 5, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein. “Heteroaryloxy” is a heteroaryl group as described bound to the group it substituted via an oxygen, —O—, linker.

“Heteroarylalkyl” is an alkyl group as described herein substituted with a heteroaryl group as described herein.

“Arylalkyl” is an alkyl group as described herein substituted with an aryl group as described herein.

“Heterocycloalkyl” is an alkyl group as described herein substituted with a heterocyclo group as described herein.

The term “heteroalkyl” refers to an alkyl, alkenyl, alkynyl, or haloalkyl moiety as defined herein wherein a CH₂ group is either replaced by a heteroatom or a carbon atom is substituted with a heteroatom for example, an amine, carbonyl, carboxy, oxo, thio, phosphate, phosphonate, nitrogen, phosphorus, silicon, or boron. In one embodiment, the only heteroatom is nitrogen. In one embodiment, the only heteroatom is oxygen. In one embodiment, the only heteroatom is sulfur. In one embodiment, “heteroalkyl” is used to indicate a heteroaliphatic group (cyclic, acyclic, substituted, unsubstituted, branched or unbranched) having 1-20 carbon atoms. Nonlimiting examples of heteroalkyl moieties include polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a carrier. The present invention includes pharmaceutical compositions of the described compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed protein stabilizing compound in which the parent protein stabilizing compound is modified by making inorganic and organic, pharmaceutically acceptable, acid or base addition salts thereof. The salts of the present protein stabilizing compounds can be synthesized from a parent protein stabilizing compound that contains a basic or acidic moiety by conventional chemical methods. Generally, such salts can be prepared by reacting free acid forms of these protein stabilizing compounds with a stoichiometric amount of the appropriate base (such as Na, Ca, Mg, or K hydroxide, carbonate, bicarbonate, or the like), or by reacting free base forms of these protein stabilizing compounds with a stoichiometric amount of the appropriate acid. Such reactions are typically carried out in water or in an organic solvent, or in a mixture of the two. Salts of the present protein stabilizing compounds further include solvates of the protein stabilizing compounds and of the protein stabilizing compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent protein stabilizing compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active protein stabilizing compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host is a human. A “patient” or “host” or “subject” also refers to for example, a mammal, primate (e.g., human), cow, sheep, goat, horse, dog, cat, rabbit, rat, mice, bird and the like.

A “therapeutically effective amount” of a compound, pharmaceutical composition, or combination of this invention means an amount effective, when administered to a host, provides a therapeutic benefit such as an amelioration of symptoms or reduction or diminution of the disease itself.

### Pharmaceutical Compositions

A protein stabilizing compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the Target Ubiquitinated Protein, as described herein or otherwise well-known for that Target Ubiquitinated Protein.

A protein stabilizing compound of the present invention can be administered in any manner that allows the protein stabilizing compound to stabilize the Target Ubiquitinated Protein. As such, examples of methods to deliver a protein stabilizing compound of the present invention include, but are not limited to, oral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, a protein stabilizing compound of the present invention is provided in a liquid dosage form, a solid dosage form, a gel, particle, etc.

In certain embodiments the protein stabilizing compound of the present invention is administered subcutaneously. Typically, the protein stabilizing compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the protein stabilizing compound of the present invention is administered intravenously. Typically, if administered intravenously, the protein stabilizing compound will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

Therefore, the disclosure provides pharmaceutical compositions comprising an effective amount of protein stabilizing compound or its pharmaceutically acceptable salt together with at least one pharmaceutically acceptable carrier for any appropriate use thereof. The pharmaceutical composition may contain a protein stabilizing compound or salt as the only active agent, or, in an alternative embodiment, the protein stabilizing compound and at least one additional active agent.

The term “pharmaceutically acceptable salt” as used herein refers to a salt of the described protein stabilizing compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed protein stabilizing compounds. These salts can be prepared during the final isolation and purification of the protein stabilizing compounds or by separately reacting the purified protein stabilizing compound in its free form with a suitable organic or inorganic acid and then isolating the salt thus formed. Basic protein stabilizing compounds are capable of forming a wide variety of different salts with various inorganic and organic acids. Acid addition salts of the basic protein stabilizing compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic protein stabilizing compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active protein stabilizing compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active protein stabilizing compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, a protein stabilizing compound disclosed herein or used as described is administered once a day (QD), twice a day (BID), or three times a day (TID). In some embodiments, a protein stabilizing compound disclosed herein or used as described is administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the protein stabilizing compound of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the protein stabilizing compound is sufficient to provide a practical quantity of material for administration per unit dose of the protein stabilizing compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution, citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the protein stabilizing compound of the present invention.

The pharmaceutical compositions/combinations can be formulated for oral administration. These compositions can contain any amount of active protein stabilizing compound that achieves the desired result, for example between 0.1 and 99 weight % (wt. %) of the protein stabilizing compound and usually at least about 1 wt. % of the protein stabilizing compound. Some embodiments contain from about 25 wt. % to about 50 wt. % or from about 5 wt. % to about 75 wt. % of the protein stabilizing compound. Enteric coated oral tablets may also be used to enhance bioavailability of the protein stabilizing compound for an oral route of administration.

Formulations suitable for rectal administration are typically presented as unit dose suppositories. These may be prepared by admixing the active protein stabilizing compound with one or more conventional solid carriers, for example, cocoa butter, and then shaping the resulting mixture.

### Target Ubiquitinated Protein and Ubiquitinated Protein Targeting Ligands

The compounds described herein include a Ubiquitinated Protein Targeting Ligand. In certain embodiments, the Ubiquitinated Protein Targeting Ligand is a small organic molecule (e.g. not an inorganic substance or peptide) that binds to the Target Ubiquitinated Protein adequately to facilitate deubiquitination. In certain embodiments of the invention, the Ubiquitinated Protein Targeting Ligand is a is a peptide or oligonucleotide that binds to the Target Ubiquitinated Protein adequately to facilitate deubiquitination. In certain embodiments the Ubiquitinated Protein Targeting Ligand is a pharmaceutically active compound or a fragment thereof that binds to the Target Ubiquitinated Protein (for example an approved drug or a compound in development with known binding affinity for the Target Ubiquitinated Protein in either the ubiquitinated or nonubiquitinated form). A plethora of illustrative nonlimiting examples or Ubiquitinated Protein Targeting Ligands for use in the present invention are provided in the Detailed Description and Figures. Additional Ubiquitinated Protein Targeting Ligand are known in the art.

In certain embodiments the Ubiquitinated Protein Targeting Ligand binds the Target Ubiquitinated Protein before it is ubiquitinated and prevents ubiquitination or removes ubiquitins that are added subsequently. In other embodiments the Ubiquitinated Protein Targeting Ligand binds the Target Ubiquitinated Protein after it is ubiquitinated and prevents further ubiquitination or removes ubiquitins that are added subsequently.

Where proteins are referred to both wild type and non-wild type versions of the protein are contemplated unless excluded by context. For example, where the Target Ubiquitinated Protein is CFTR the CFTR may be wild-type or have one or more mutations.

In certain embodiments the Target Ubiquitinated Protein is a mediator of a renal disease, for example CLDN16, CLDN19, FXYD2, UMOD, SLC12A3, SLC4A1, SCNN1B, SCNN1G, AVPR2, AQP2, CFTR, GLA, COL4A3, COL4A4, COL4A5, COL4A1, ACTN4, TRPC6, INF2, MYO1E, NPHS1, NPHS2, LAMB2, CTNS, SLC3A1, CLCN5, OCRL, SLC34A3, PHEX, FGF23, DMP1, OCRL, SLC4A4, SLC5A2, SLC5A1, SLC12A1, KCNJ1, BSND.

Non-limiting examples of renal disease include hypomagnesaemia type 2, hypomagnesaemia type 3, hypomagnesaemia type 5, uromodulin-associated kidney disease, gitelman syndrome, distal renal tubular acidosis, liddle syndrome, nephrogenic diabestes insipidus, cystic fibrosis, fabry disease, Alport syndrome, hereditary angiopathy with nephropathy aneurysms and muscle crams (HANAC), focal segmental glomerulosclerosis 1, focal segmental glomerulosclerosis 2, focal segmental glomerulosclerosis 5, focal segmental glomerulosclerosis 6, nephrotic syndrome type 1, nephrotic syndrome type 2, Pierson syndrome, cystinosis, cystinuria type A, Dent's disease 1, Dent's disease 2, hypophosphataemic rickets with hypercalciuria, hypophosphataemic rickets, Lowe syndrome, proimal renal tubular acidosis, renal glucosuria, Bartter syndrome antenatal type 1, Bartter syndrome antenatal type 2, Bartter syndrome type 4,

As used herein 4-character identifier referring to crystal structures are RCS Protein Data Base (PDB) crystal structure identifiers and 3-character identifiers referring to ligands are PDB ligand identifiers. The skilled artisan will recognize that these codes can be entered into the PDB to view crystal structures of the referenced proteins and ligands. These crystal structures provide direction for where to attach the linker to the targeting ligand while maintaining binding efficacy. For example 602P refers to a crystal structure of cystic fibrosis transmembrane conductance regulator protein (CFTR) in complex with ivacaftor. By entering 602P into the PDB (for example at https://www.rcsb.org/) the crystal structure can be viewed.

### CFTR

In certain embodiments the protein stabilizing compound of the present invention includes a CFTR targeting ligand and can be used in the treatment of a CFTR mediated disease such as cystic fibrosis, male infertility, polycystic kidney disease, obstructive lung disease, intestinal obstruction syndromes, liver dysfunction, exocrine and endocrine pancreatic dysfunction, or secretory diarrhea.

CFTR is a glycoprotein with 1480 amino acids and is classified as an ABC (ATP-binding cassette) transporter. The cystic fibrosis transmembrane conductance regulator protein (CFTR) is a cAMP activated chloride ion (Cr) channel responsible for Cl− transport. CFTR is expressed in epithelial cells in mammalian airways, intestine, pancreas and testis. It is there where CFTR provides a pathway for the movement of Cl− ions across the apical membrane and a key point at which to regulate the rate of transepithelial salt and water transport. Hormones, such as a β-adrenergic agonist, or toxins, such as cholera toxin, lead to an increase in cAMP, activation of cAMP-dependent protein kinase, and phosphorylation of the CFTR Cl− channel, which causes the channel to open. An increase in the concentration of Ca2+ in a cell can also activate different apical membrane channels. Phosphorylation by protein kinase C can either open or shut Cl− channels in the apical membrane.

The CFTR protein consists of five domains. There are two nucleotide binding domains (NBD1 and NBD2), regulatory domain (RD) and two transmembrane domains (TMD1 and TMD2). The protein activity is regulated by cAMP-dependent Protein Kinase (PKA) which catalyze phosphorylation of regulatory domain (RD) and also binding of two ATP molecules to NBD1 and NBD2 domains. Nonlimiting examples of CFTR mutant proteins include ΔF508 CFTR, G551D-CFTR, G1349D-CFTR, D1152H-CFTR, E56K, P67L, E92K, L206W. These mutations cause CFTR to be dysfunctional (e.g. operate with less activity that WT CFTR).

Dysfunction of CFTR is associated with a wide spectrum of disease, including cystic fibrosis (CF) and with some forms of male infertility, polycystic kidney disease, obstructive lung disease, intestinal obstruction syndromes, liver dysfunction, exocrine and endocrine pancreatic dysfunction and secretory diarrhea. CF is a hereditary disease that mainly affects the lungs and digestive system, causing progressive disability and early death. With an average life expectancy of around 31 years, CF is one of the most common life-shortening, childhood-onset inherited diseases. This disease is caused by mutation of the gene encoding CFTR, and is autosomal recessive.

In certain embodiments, the Ubiquitinated Protein Targeting Ligand is a ligand for CFTR selected from a small molecule, polypeptide, peptidomimetic, antibody, antibody fragment, antibody-like protein, and nucleic acid. In some embodiments, the CFTR Targeting Ligand is a corrector agent (e.g., a ligand that activates CFTR or rescues CFTR or mutant CFTR from degradation).

In certain embodiments, CFTR correctors are molecules that correct one or more defects by rescuing proteins from endoplasmic reticulum degradation, improving trafficking of CFTR to the cell surface, and/or inhibiting proteins that are involved in the recycling of CFTR in the cell membrane. Several correctors have been identified using high throughput assays (O'Sullivan & Freedman (2009) Lancet 373:1991-2004).

In certain embodiments, CFTR corrector compound is selected from corr-4a (Pedemonte, et al. (2005) J. Clin. Invest. 115:2564) and Lumacaftor (VX-809), which partially alleviate the folding defect and allows some AF508-CFTR to reach the apical membrane (Van Goor, et al. (2009) Pediatr. Pulmonol. 44:S154-S155; Van Goor, et al. (2011) Proc. Natl. Acad. Sci. USA 108:18843-18848).

In certain embodiments the CFTR Targeting Ligand is a compound described in WO2016077413A1, WO2010048125A2, or WO2013070529A1.

In certain embodiments the CFTR Targeting Ligand is a polypeptide. In certain embodiments the polypeptide is at least about 3, 5, 10, 15, 20, 25, 30, 35, 40, 45, 50, 55, 60, 65, 70, 75, 80, 90, 100, 110, 120, 130, 140, 150, 175, 200, 225 or 250 amino acids in length. In certain embodiments, the polypeptide is about 5-10, 5-25, 5-50, 5-75, 5-100, 5-150 or 5-200 amino acids in length. In certain embodiments, the polypeptide is membrane permeable.

In certain embodiments, the CFTR Targeting Ligand comprises a chimeric polypeptide which further comprises one or more fusion domains. Nonlimiting examples of chimeric polypeptides comprising one or more fusion domains include polyhistidine, Glu-Glu, glutathione S transferase (GST), thioredoxin, protein A, protein G, and an immunoglobulin heavy chain constant region (Fc), maltose binding protein (MBP).

In certain embodiments, the CFTR Targeting Ligand comprises a chimeric polypeptide comprising a first portion that is a polypeptide corrector agent, and a second portion that serves as a targeting moiety. In certain embodiments, the targeting moiety targets a subject's lungs, pancreas, liver, intestines, sinuses, and/or sex organs.

In certain embodiments, the CFTR Targeting Ligand may further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the CFTR Targeting Ligand may contain non-amino acid elements, such as lipids, poly- or mono-saccharide, and phosphates.

In certain embodiments, the CFTR Targeting Ligand is a potentiator which enhances the activity of CFTR that is correctly located at the cell membrane. CFTR potentiators are particularly useful in the treatment of subjects with class III mutations.

Non-limiting examples of CFTR potentiators include, but are not limited to, certain flavones and isoflavones, such as genistein, which are capable of stimulating CFTR-mediated chloride transport in epithelial tissues in a cyclic-AMP independent manner (See U.S. Pat. No. 6,329,422, incorporated herein by reference in its entirety); phenylglycine-01 (2-[(2-1H-indol-3-yl-acetyl)-methylamino]-N-(4-isopropylphenyl)-2-phenylacetamide); felodipine (Ethylmethyl-4-(2,3-dichlorophenyl)-2,6-dimethyl-1,4-dihydro-3, 5-pyridinedicarboxylate); sulfonamide SF-01 (6-(ethylphenylsulfamoyl)-4-oxo-1,4-dihydroquinoline-3-carboxylic acid cycloheptylamide); UCCF-152 (3-[2-(benzyloxy) phenyl]-5-(chloromethyl) isoxazole), and Ivacaftor (VX-770; N-(2,-Di-tert-butyl-5-hydroxyphenyl)-4-oxo-1, 4-dihydroquinoline-3-carboxamide).

In certain embodiments, the compounds described herein is used in addition to a dual corrector and potentiator activities. In certain embodiments, non-limiting examples of dual correctors and potentiators include VRT-532 (3-(2-hydroxy-5-methylphenyl)-5-phenylpyrazole) and cyanoquinolines such as N-(2-((3-Cyano-5,7-dimethylquinolin-2-yl) amino) ethyl)-3-methoxybenzamide (CoPo-2), hybrid bithiazole-phenylglycine corrector-potentiators which, when cleaved by intestinal enzymes, yield an active bithiazole corrector and phenylglycine potentiator (Mills, et al. (2010) Bioorg. Med. Chem. Lett. 20:87-91). The only FDA-approved CFTR activator, VX-770, is a “potentiator” developed by the treatment of CF by correcting the channel gating of certain CFTR mutations.

In certain embodiments, the CFTR Targeting Ligand is selected from Ataluren (3˜[5-(2-Fluorophenyl)-1, 2, 4-oxadiazol-3-yl] benzoic acid), Lumacaftor (VX-809; 3-{6-{[1-(2, 2-difluoro-1, 3-benzodioxol-5-yl) cyclopropanecarbonyl] amino}-3-methylpyridin-2-yl}benzoic acid), ivacaftor, VX-661, FDL169, N91115, QBW251, Riociguat, QR-010, lumacaftor, GLPG222, VX-152, VX-440, VX-445, VX-561 (aka CTP-656), VX-659, PTI-428, PTI-801, and PTI-808.

In certain embodiments a compound described herein stabilizes wildtype CFTR and/or mutant CFTR that has been ubiquitinated and thus tagged for proteasomal degradation and removes enough ubiquitins to allow the compound to be trafficked back to the cell membrane and thus restore function.

In certain embodiments the protein stabilizing compound contains lumacaftor or a derivative or fragment thereof:

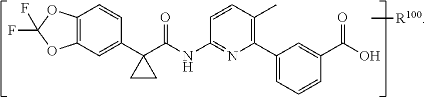

In certain embodiments the protein stabilizing compound contains ivacaftor or a derivative or fragment thereof:

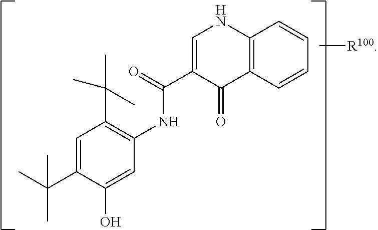

In certain embodiments the protein stabilizing compound contains tezacaftor or a derivative or fragment thereof:

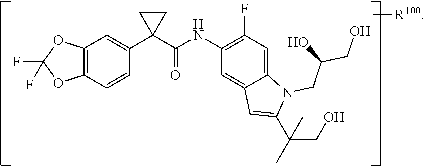

A compound described herein with a CFTR Targeting Ligand removes ubiquitin from Ubiquitinated CFTR in a manner that stabilizes CFTR and in some embodiments restore the CFTR's function. For example, when the Target Ubiquitinated CFTR has a mutation that causes it to incorrectly fold, a compound of the present invention with a CFTR Targeting Ligand that is a corrector may increase its activity by removing ubiquitins and correcting its folding so that it may function correctly. When the Target Ubiquitinated CFTR has a mutation that causes it to less effectively function as a gating and conduction protein, a compound of the present invention with a CFTR Targeting Ligand that is a potentiator may increase its activity by removing ubiquitins and potentiating the protein.

In certain embodiments a compound of the present invention with a CFTR Targeting Ligand or a pharmaceutically acceptable salt thereof is used in combination with a potentiator of CFTR or a pharmaceutically acceptable salt thereof to treat cystic fibrosis. In certain embodiments a compound of the present invention with a CFTR Targeting Ligand or a pharmaceutically acceptable salt thereof is used in combination with a corrector of CFTR or a pharmaceutically acceptable salt thereof to treat cystic fibrosis. Non-limiting examples of CFTR potentiators include ivacaftor, deutivacaftor, and ABBV-974. Non-limiting examples of CFTR correctors include lumacaftor, tezacaftor, posenacaftor, olacaftor, bamocaftor, and elexacaftor. In certain embodiments a compound of the present invention has a CFTR Targeting Ligand that is a potentiator and the compound is used in combination with a CFTR corrector. In certain embodiments a compound of the present invention has a CFTR Targeting Ligand that is a corrector and the compound is used in combination with a CFTR potentiator.

### PAH

In certain embodiments the protein stabilizing compound of the present invention includes a PAH targeting ligand and can be used in the treatment of a PAH-mediated disease such as PAH deficiency (e.g. phenylketonuria (PKU), non-PKU hyperphenylalaninemia (HPA), or variant PKU).

Phenylalanine hydroxylase (PAH) catalyzes the hydroxylation of phenylalanine to tyrosine. It exists as an equilibrium of monomeric and dimeric forms (monomer size 51.9 kDa) and contains a catalytic nonheme iron in the catalytic site. The hydroxylation proceeds through an iron (IV) oxo intermediate generated by the tetrahydrobiopterin cofactor. Although phenylalanine is utilized in protein synthesis, most of the dietary phenylalanine is broken down into carbon dioxide and water over a series of steps. The rate limiting step in phenylalanine catabolism is hydroxylation to tyrosine, which provides a synthetic handle for later enzymes to break down the aromatic side chain. Deficiencies in PAH are inherited in an autosomal recessive manner, and lead to a dangerous buildup of phenylalanine causing seizures, intellectual disability, and microcephaly in infected children. Preventing symptomatic PKU requires strict adherence to a physician prescribed diet to reduce the intake of the amino acid phenylalanine. Additional supplementation with tyrosine and other downstream metabolites is required for proper development.

Non-limiting examples of crystal structures of PAH with Protein Recognition Moieties include 4JPY, 1LTZ, 4ANP, 1KWO, 1TG2, 3PAH, 4PAH, 5PAH, 6PAH, and 5JK5.

In certain embodiments the PAH Targeting Ligand is selected from

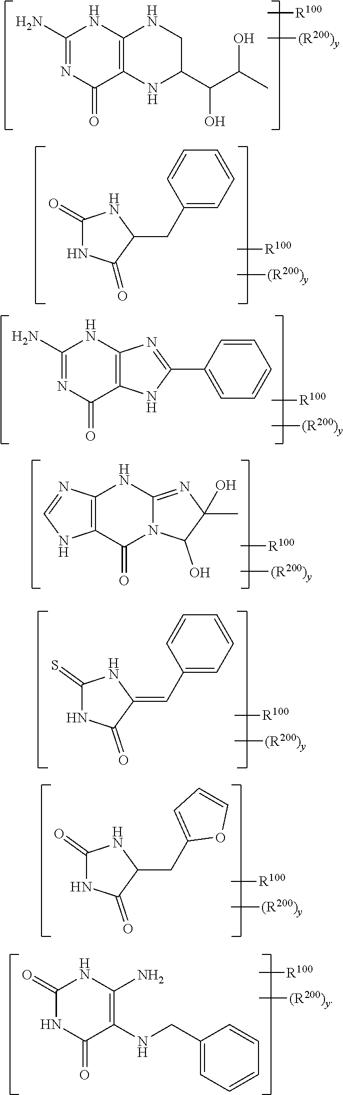

### ABCA4

In certain embodiments the protein stabilizing compound of the present invention includes a ABCA4 Targeting Ligand and can be used in the treatment of a ABCA4-mediated disease such as Stargardt disease or retinal degeneration.

ATP-binding cassette, sub family A, member 4 (ABCA4) is a transporter protein expressed in rod photoreceptors of the eye. The protein consists of two extracellular domains, two intracellular domains, and two transmembrane domains. Upon binding of ATP to the intracellular nucleotide binding site, the transmembrane domain changes shape to facilitate transport of retinoid ligands. As retinoids degrade, they form covalent adducts with phosphatidoethanolamine which generates a charged species that is recognized by ABCA4. In knockout mice, photobleaching the retina with strong light causes a significant buildup of the N-retinyl-phosphatidylethanolamine. Toxic levels of this molecule cause age-related macular degeneration. In humans, mutations of ABCA4 lead to Stargardt macular dystrophy, a juvenile macular degeneration in which the photoreceptors of the macula die off causing central blindness.

In certain embodiments the protein stabilizing compound contains lumacaftor or a derivative or fragment thereof and can be used for the treatment of an ABCA4-mediated disorder such as Stargardt disease:

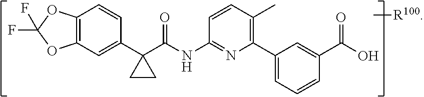

Non-limiting examples of crystal structures of ABCA4 with Protein Recognition Moieties include 7LKP and 7LKZ.

### Rhodopsin

In certain embodiments the protein stabilizing compound of the present invention includes a rhodopsin Targeting Ligand and can be used in the treatment of a rhodopsin-mediated disease such as retinitis pigmentosa, leber congenital amaurosis, or congenital night blindness.

Rhodopsin is a G-protein-coupled receptor (GCPR) expressed in rod cells of the retina and is responsible for vision in low light conditions. Within the seven transmembrane domains lies a photosensitive molecule, retinal. Upon isomerization of the alkenes within retinal, the G protein is activated causing a cGMP messenger cascade. Many retinopathies are caused by mutations in the rhodopsin gene, causing pathological ubiquitinization of rhodopsin. Ubiquitinization of rhodopsin ultimately leads to photoreceptor apoptosis and blindness.

Non-limiting examples of crystal structures of Rhodopsin 1 with Protein Recognition Moieties include 6I9K and 5AWZ. Non-limiting examples of crystal structures of Rhodopsin with Protein Recognition Moieties include 3AYM, 1L9H, 6FK6, 6FK8, 6FK7, 6FKD, 6FKC, 6FKB, 6FKA and 5TE5. Non-limiting examples of crystal structures of Rhodopsin II with Protein Recognition Moieties include 1H2S and 3AM6.

### ABCB4

In certain embodiments the protein stabilizing compound of the present invention includes an ABCB4 Targeting Ligand and can be used in the treatment of an ABCB4-mediated disease such as progressive familial intrahepatic cholestasis (PFIC), for example PFIC3.

ATP-binding cassette 4, or multidrug resistance protein 3, is a transporter protein responsible for transfer of phosphatidylcholine into the bile ducts. The phospholipid is crucial for chaperoning the bile acid into the gut, thereby protecting the duct itself. Mutations in the gene are inherited in an autosomal recessive manner and lead to progressive familial intrahepatic cholestasis-3 (PFIC-3). Patients with PFIC-3 develop bile plugs and infarcts, as well as hepatocellular injury early in childhood. If untreated the disease progresses to liver failure and death before adolescence.

In certain embodiments, the Ubiquitinated Protein Targeting Ligand is a ligand for ABCB4 selected from a small molecule, polypeptide, peptidomimetic, antibody, antibody fragment, antibody-like protein, and nucleic acid.

### ABCB11

In certain embodiments the protein stabilizing compound of the present invention includes an ABCB11 Targeting Ligand and can be used in the treatment of an ABCB11-mediated disease such as progressive familial intrahepatic cholestasis (PFIC), for example PFIC2.

ATP-binding cassette, sub-family B member 11 (ABCB11) is a transmembrane transport protein that is responsible for bile acid homeostasis in the body. Upon binding of ATP, the triphosphate is hydrolyzed causing the transport of one molecule of cholate. Proper transport of bile acids prevents toxic buildup in hepatocytes as well as proper processing of toxins, and absorption of vitamins and fat from the diet. A deficiency in this protein causes excessive pruritis (itching), jaundice, liver cancer, leading to cirrhosis within five to ten years of life. The current treatment options are limited to invasive biliary diversion surgery or complete liver transplant.

In certain embodiments, the Ubiquitinated Protein Targeting Ligand is a ligand for ABCB11 selected from a small molecule, polypeptide, peptidomimetic, antibody, antibody fragment, antibody-like protein, and nucleic acid.

### Dystrophin

In certain embodiments the protein stabilizing compound of the present invention includes a dystrophin Targeting Ligand and can be used in the treatment of an dystrophin-mediated disease such as muscular dystrophy for example Duchenne muscular dystrophy.

Dystrophin is a crucial structural protein responsible for the attachment of muscle cytoskeleton to the surrounding extracellular matrix. The protein is localized between the muscular cell plasma membrane (sarcolemma) and the myofiber, allowing it to attach the muscle fibers to the plasma membrane. This is the fundamental connection between tendons and the motive part of the muscular system. Due to its presence on the X chromosome, deficiencies in this gene are inherited in an X-linked recessive manner and most affected individuals are male. Dystrophin mutations cause a range of diseases known as muscular dystrophy, including Duchenne muscular dystrophy.

Antisense oligonucleotides have been examined as potential therapies, however none have been able to establish statistically significant benefit. There remains tremendous unmet medical need for patients with dystrophin mutations.

In certain embodiments, the Ubiquitinated Protein Targeting Ligand is a ligand for dystrophin selected from a small molecule, polypeptide, peptidomimetic, antibody, antibody fragment, antibody-like protein, and nucleic acid.

### P27 and P27^{Kip1}

In certain embodiments the protein stabilizing compound of the present invention includes a P27 or P27^{Kip1} Targeting Ligand and can be used in the treatment of a P27 or P27^{Kip1}-mediated disease such as a cancer for example oro-pharyngo-laryngeal cancer, oesophageal cancer, gastric cancer, colon cancer, biliary tract cancer, lung cancer, melanoma, glioma, glioblastoma, breast cancer, renal cell cancer, prostate cancer, transitional cell cancer, cervix cancer, endometrial cancer, ovarian cancer, Kaposi sarcoma, soft tissue sarcoma, lymphoma, or leukemia.

P27 (encoded by the CDKN1B gene) is a cell cycle inhibitor that prevents rapid cell division. Transcription of CDKN1B is activated by FoxO, which then serves as a nuclear localization signal for P27 and decreases the levels of a P27 degrading protein COPS5. This process occurs predominanly during quiescence and early G1. To enter the cell cycle, P27 is ubiquitinated by two different proteins, SCFSKP2 kinase associate protein 1 as well as the KIP1 ubiquitylation promoting complex. These complexes polyubiquitinate P27, causing its degradation and release of inhibitory signal. Once the levels of P27 decrease, the cell begins to replicate.

Many cancers are a result of dysfunction in the synthesis, localization, or degradation of P27 and stabilizing its presence is an attractive strategy to limit replication.

Non-limiting examples of crystal structures of P27KIP1 with Protein Recognition Moieties include 3A99.

In certain embodiments the P27 or P27^{Kip1} Targeting Ligand is selected from

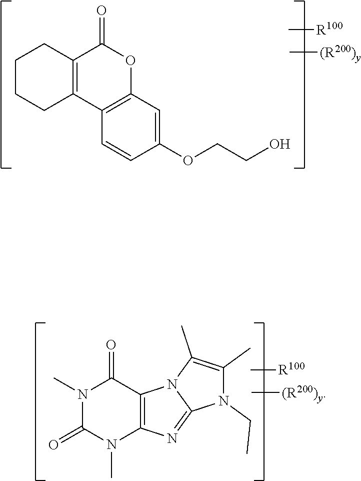

### PDCD4

In certain embodiments the protein stabilizing compound of the present invention includes a PDCD4 Targeting Ligand and can be used in the treatment of a PDCD4-mediated disease such as a cancer for example pregnancy-associated breast cancer, pancreatic cancer, lung cancer, and primary lung cancer.

Programmed cell death protein 4 (PDCD4) is a tumor suppressor protein that regulates transcription in addition to cell proliferation and tumor metastasis. PDCD4 suppresses the expression of protumor kinases JNK and MAP4K1, both proteins responsible for cell cycle initiation. PDCD4 is phosphorylated by S6 kinase (downstream of PI3K-Akt-mTOR signaling) at which point it is ubiquitinylated and then degraded. Removal of PDCD4 either through siRNA knockdown or knockout experiments shows a phenotype of aggressive cellular proliferation. In certain embodiments the PDCD4 Targeting Ligand is a ligand described in Frankel et al. J. Biol. Chem. 2008, 283(2): 1026-1033, for example SEQ ID. 1 UAGCUUAUCAGACUGAUGUUGA.

### P53 Tumor Suppressor

In certain embodiments the protein stabilizing compound of the present invention includes a p53 Targeting Ligand and can be used in the treatment of a p53-mediated disease such as a cancer.

P53 is a 43.7 kDa protein that is responsible for tumor suppression in multicellular vertebrates, and is mutated in over 50% of cancers. It plays multiple roles in preventing the development in cancers, including activation of DNA repair proteins, pausing the cell cycle to allow DNA repair to occur, and initiating apoptosis if the DNA damage is unrepairable. If p53 is mutated or otherwise inoperable, then p21 will not be produced in sufficient quantity to halt DNA replication and cell division. This allows cells with damaged DNA, a hallmark of cancer, to divide uncontrolled. In cells that are unstressed, p53 is produced but rapidly degraded through ubiquitination via Mdm2. However, when cells are stressed, the ubiquitin is cleaved and p53 is allowed to halt replication for the necessary repair processes. Given the significance of aberrant p53 regulation in cancer, it is advantageous to be able to deubiquitinate p53 to slow the growth of tumors.

In certain embodiments the p53 Targeting Ligand targets a p53 mutant protein. For example an amino-terminal (AT) mutation, oligomerization domain (OD) mutation, DBD mutation, or loss of function mutation. In certain embodiments the p53 Targeting Ligand targets p53 with one or more mutations selected from Q136P, Y234H V272M, F270V, P278A, R213L, Y126H, T253N, T253I, R158L, Q136E, P142F, A129D, L194R, R110P, V172G, C176F, I254N, K305R, E285D, T155P, H296D, E258G, G279V, T211A, R213P, C229Y, I232F, E294K, P152R, R196P, M160T, N131S, N131H, K139N, L330H, Y220N, Y220C, E298Q, D148E, L64R, E224D, H168P, N263H, K320N, S227C, E286D, K292T, V203A, M237R, F212L, K132Q, Y236S, Y126S, Q136H, E221A, I232S, Y163H, P190T, C182Y, P142L, Y163S, V218E, I195S, V272A, and/or S106R. In certain embodiments the p53 Targeting Ligand targets Y220C p53 mutant.

Non-limiting examples of crystal structures of p53 with Protein Recognition Moieties include, 5O1C, 5O1F, 6GGA, 6GGE, 6GGC, 2VUK, 6GGN, 3ZME, 4AGN, 4AGO, 4AGM, 4AGP, 4AGQ, 5G4O, and 5ABA.

c-Myc

In certain embodiments the protein stabilizing compound of the present invention includes a c-Myc Targeting Ligand and can be used in the treatment of a c-Myc-mediated disease such as a cancer. Non-limiting examples of crystal structures of c-Myc with Protein Recognition Moieties include 2L7V, 5W77, 6JJ0, 2N6C, 6UIF, 6UHZ, 6UHY, 6UJ4, 6UIK, 6UOZ.

### MSH2

In certain embodiments the protein stabilizing compound of the present invention includes a MSH2 Targeting Ligand and can be used in the treatment of a MSH2-mediated disease such as a cancer, lynch disorder, colon cancer, or endometrial cancer.

DNA mismatch repair protein MSH2 is a tumor suppressor protein that forms a heterodimer with MSH6 which binds to DNA mismatches, stimulating repair. It is involved in transcription coupled repair, homologous recombination, and base excision repair. Loss of the mismatch repair system leads to microsatellite instability, an important component of colon cancer as well as others.

Non-limiting examples of crystal structures of MSH2 with Protein Recognition Moieties include 2O8E.

### RIPK1

In certain embodiments the protein stabilizing compound of the present invention includes a RIPK1 Targeting Ligand and can be used in the treatment of a RIPK1-mediated disease such as an inflammatory disorder, an immune disorder, an inflammatory immune disorder, cancer, or melanoma.

Receptor-interacting protein kinase 1 (RIPK1) is a serine/threonine kinase that is a crucial regulator of TNF-mediated apoptosis. RIPK1 kinase activation has been seen in samples of autoimmune and neurodegenerative conditions. RIPK1 activation begins with polyubiquitination, which then promotes the recruitment of TAK1 kinase and LUBAC complex. This complex in turn leads to necrosis and the generation of proinflammatory signaling.

Non-limiting examples of crystal structures of RIPK1 with Protein Recognition Moieties include 6NW2, 6NYH, 6AC5, 6ACI, 6C4D, 6C3E, 6O5Z, 6ZZ1, 5KO1, 4ITH, 4ITI, 4ITJ, 4NEU, 5HX6, 6OCQ, 6R5F, 5TX5, 6RLN, and 6HHO.

### RIPK2

In certain embodiments the protein stabilizing compound of the present invention includes a RIPK2 Targeting Ligand and can be used in the treatment of a RIPK2-mediated disease such as an inflammatory disorder, an immune disorder, an inflammatory immune disorder, cancer, or melanoma.

Receptor-interacting protein kinase 2 (RIPK2) is a serine/threonine/tyrosine kinase that is involved in immunological signaling as well as an inducer of apoptosis. Once ubiquitinated, RIPK2 recruits MAP3K7 to NEMO and this stimulates the release of NF-kappa-B, ultimately leading to activation of genes involved in cell proliferation and protection against apoptosis.

Non-limiting examples of crystal structures of RIPK1 with Protein Recognition Moieties include 6FU5, 4C8B, 5W5O, 5W5J, 6ESO, 6S1F, SYRN, 6SZJ, 6SZE, 6HMX, 6GGS, 6RNA, 6RN8, 5NG2, 5NGO, 5J7B, 5J79, 5AR8, 5AR7, 5AR5, and 5AR4.

### BAX

In certain embodiments the protein stabilizing compound of the present invention includes a BAX Targeting Ligand and can be used in the treatment of a BAX-mediated disease such as cancer, neurological disorders, neurodegenerative diseases, or inflammatory diseases.

Apoptosis regulator BAX (Bcl-2 like protein 4) is a member of the Bcl-2 family of proteins. BAX acts as an apoptotic activator through depletion of membrane potential in the mitochondria. The protein is located in the mitochondrial outer membrane. BAX deletions have been implicated in progressive neurological disorders that lead to ataxia and granule cell apoptosis. Furthermore BAX is critical in maintaining the number of B cells in both immature and mature stages.

Non-limiting examples of crystal structures of BAX with Protein Recognition Moieties include 4S0O, 3PK1, 4SOP, 4BD5, 5W63, 5W62, 4BD8, 4BD7, 5W61, 5W60, 4BD2, 3PL7.

### Alpha-Antitrypsin

In certain embodiments the protein stabilizing compound of the present invention includes an alpha antitrypsin Targeting Ligand and can be used in the treatment of an alpha antitrypsin-mediated disease such as chronic obstructive pulmonary disease, emphysema, jaundice, and liver related diseases including hepatitis and cirrhosis,

Alpha antitrypsin, encoded by the gene SERPINA1, is a serine protease inhibitor. This protein is produced by the liver and inhibits the digestive enzyme trypsin as well as neutrophil elastase. When there is insufficient alpha antitrypsin, the immune system attacks the alveolar sacs in the lungs which leads to difficulty breathing, COPD, and emphysema.

Non-limiting examples of crystal structures of alpha antitrypsin with Protein Recognition Moieties include 1D5S, 8API, 3DRM, 3DRU, 3CWL, 2QUG, 9API, 7API, 3TIP, 1HP7, 3CWM, 5IO1, 1QLP, 3NE4, 1ATU, 1PSI, 1QMB, 1KCT, 3DNF, 3NDD, 7AEL, 1IZ2, 1OO8, 1OPH, and 1EZX,

### PKLR

In certain embodiments the protein stabilizing compound of the present invention includes a PKLR Targeting Ligand and can be used in the treatment of a PKLR-mediated disease such as chronic hereditary nonspherocytic hemolytic anemia, jaundice, fatigue, dyspnea, Gilbert syndrome, and bone fractures.

PKLR (pyruvate kinase L/R) is a protein that catalyzes the transphosphorylation of phosphoenolpyruvate into pyruvate and ATP. This is the rate limiting step in glycolysis and leads to a lack of ATP in red blood cells. The red blood cells dehydrate and form altered shapes, which leads to hemolytic anemia.

Non-limiting examples of crystal structures of PKLR with Protein Recognition Moieties include 6NN4, 6ECH, 6NN8, 6ECK, 2VGI, 2VGG, 2VGF, 2VGB, 6NN7, 6NN5 4IP7, and 4TMA,

### KEAP1

In certain embodiments the protein stabilizing compound of the present invention includes a KEAP1 Targeting Ligand and can be used in the treatment of a KEAP1-mediated disease such as inflammation, chronic kidney disease, hepatocellular carcinoma and lung cancer.

KEAP1 (Kelch-like ECH-associated protein 1) regulates the activity of a BCR E3 ubiquitin ligase complex. This protein complex is responsible for responding to oxidative stress by regulating the expression of cytoprotective genes. The protein has four domains, including one domain responsible for stress signaling. This domain contains a number of cysteine residues which undergo Michael addition to reactive electrophilic species in the cell, activating KEAP1.

Non-limiting examples of crystal structures of KEAP1 with Protein Recognition Moieties include 6LRZ, 7C60, 7C5E, 2Z32, 5FZN, 5FZJ, 5FNU, 5FNT, 5FNS, 5FNR, 5FNQ, 1X2J, 4CXT, 6ZEZ, 4CXJ, 7K2M, 7K2L, 7K2J, 7K2I, 6ZF8, 6ZF7, 6ZF6, 6ZF5, 6ZF4, 6ZF3, 6ZF2, 6ZF1, 6ZF0, 6ZEY, 6SP4, 6SP1, 5CGJ, 4IFN, 4IFJ, IU6D, 7K2S, 7K2R, 7K2Q, 7K2P, 7K20, 7K2N, 7K2H, 7K2G AND 6ZEX.

### IRAK4

In certain embodiments the protein stabilizing compound of the present invention includes a IRAK4 Targeting Ligand and can be used in the treatment of a IRAK4-mediated disease such as inflammation, infectious disease, autoimmune disease, rheumatoid arthritis and inflammatory bowel disease.

IRAK4 (interleukin-1 receptor-associated kinase 4) is a protein kinase within the toll-like receptor pathway (TLR). IRAK4 activity is required for activation of NF-kappa-B and activation of the mitogen activated protein kinase pathway that induces the cell cycle. The protein is a crucial component to an organism's response to IL-1. Without IRAK4, the animal does not adequately sense the presence of viruses or bacteria and set off the appropriate innate immune response of cytokines and chemokines. In human patients, TRAK4 deficiency presents as a defective immune system.

Non-limiting examples of crystal structures of IRAK4 with Protein Recognition Moieties include

### Methods of Treatment

A protein stabilizing compound described herein can be used to treat a disorder mediated by a Target Ubiquitinated Protein. For example, when restoring the function of the Target Ubiquitinated Protein ameliorates a cancer than the protein stabilizing compound can be used in the treatment of that cancer.

Exemplary cancers which may be treated by a disclosed protein stabilizing compound either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the a disclosed protein stabilizing compound according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioblastoma, recurrent glioblastoma, glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma (Yu et al., “DNA damage induces cdk2 protein levels and histone H2B phosphorylation in SH-SY5Y neuroblastoma cells”, J Alzheimer's Dis., 2005 September; 8(1):7-21).

Additional, non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CVL), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

In certain embodiments the disorder is a renal disease.

Non-limiting examples of renal disease include hypomagnesaemia type 2, hypomagnesaemia type 3, hypomagnesaemia type 5, uromodulin-associated kidney disease, gitelman syndrome, distal renal tubular acidosis, liddle syndrome, nephrogenic diabestes insipidus, cystic fibrosis, fabry disease, Alport syndrome, hereditary angiopathy with nephropathy aneurysms and muscle crams (HANAC), focal segmental glomerulosclerosis 1, focal segmental glomerulosclerosis 2, focal segmental glomerulosclerosis 5, focal segmental glomerulosclerosis 6, nephrotic syndrome type 1, nephrotic syndrome type 2, Pierson syndrome, cystinosis, cystinuria type A, Dent's disease 1, Dent's disease 2, hypophosphataemic rickets with hypercalciuria, hypophosphataemic rickets, Lowe syndrome, proimal renal tubular acidosis, renal glucosuria, Bartter syndrome antenatal type 1, Bartter syndrome antenatal type 2, and Bartter syndrome type 4.

In certain embodiments the disorder is cystic fibrosis.

In certain embodiments the disorder is phenylketonuria (PKU), non-PKU hyperphenylalaninemia (HPA), or variant PKU.

In certain embodiments the disorder is Stargardt disease or retinal degeneration.

In certain embodiments the disorder is retinitis pigmentosa, leber congenital amaurosis, or congenital night blindness.

In certain embodiments the disorder is progressive familial intrahepatic cholestasis (PFIC).

In certain embodiments the disorder is muscular dystrophy for example Duchenne muscular dystrophy.

In certain embodiments the disorder is oro-pharyngo-laryngeal cancer, oesophageal cancer, gastric cancer, colon cancer, biliary tract cancer, lung cancer, melanoma, glioma, glioblastoma, breast cancer, renal cell cancer, prostate cancer, transitional cell cancer, cervix cancer, endometrial cancer, ovarian cancer, Kaposi sarcoma, soft tissue sarcoma, lymphoma, or leukemia.

In certain embodiments the disorder is pregnancy-associated breast cancer, pancreatic cancer, lung cancer, and primary lung cancer.

In certain embodiments the disorder is inflammatory disorder, an immune disorder, an inflammatory immune disorder, cancer, or melanoma.

### Linker

In certain embodiments the USP7 Targeting Ligand and Ubiquitinated Protein Targeting Ligand are linked by a Linker group.

In certain embodiments the Linker-USP7 Targeting Ligand or Linker-Ubiquitinated Protein Targeting Ligand replaces an atom, for example a halogen, alkyl, hydroxy, alkoxy, cyano, or nitro group. For example wherein Linker is

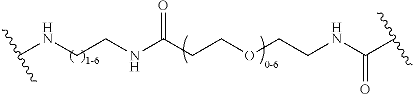

### and the USP7 Targeting Ligand is

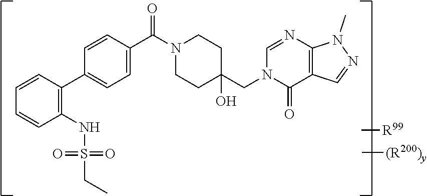

the Linker group can replace the methyl group to form the following compound:

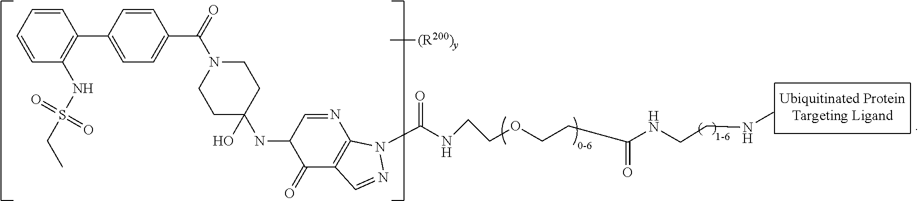

In certain embodiments the Linker-USP7 Targeting Ligand or Linker-Ubiquitinated Protein Targeting Ligand replaces a halogen.

In certain embodiments the Linker-USP7 Targeting Ligand or Linker-Ubiquitinated Protein Targeting Ligand replaces an iodine.

In certain embodiments the Linker-USP7 Targeting Ligand or Linker-Ubiquitinated Protein Targeting Ligand replaces a bromine.

In certain embodiments the Linker-USP7 Targeting Ligand or Linker-Ubiquitinated Protein Targeting Ligand replaces a chlorine.

In certain embodiments the Linker-USP7 Targeting Ligand or Linker-Ubiquitinated Protein Targeting Ligand replaces a fluorine.

In certain embodiments the Linker-USP7 Targeting Ligand or Linker-Ubiquitinated Protein Targeting Ligand replaces an alkyl.

In certain embodiments the Linker-USP7 Targeting Ligand or Linker-Ubiquitinated Protein Targeting Ligand replaces a methyl

In certain embodiments the Linker-USP7 Targeting Ligand or Linker-Ubiquitinated Protein Targeting Ligand replaces a ethyl

In certain embodiments the Linker-USP7 Targeting Ligand or Linker-Ubiquitinated Protein Targeting Ligand replaces an alkoxy.

In certain embodiments the Linker-USP7 Targeting Ligand or Linker-Ubiquitinated Protein Targeting Ligand replaces a cyano.

In certain embodiments the Linker-USP7 Targeting Ligand or Linker-Ubiquitinated Protein Targeting Ligand replaces a nitro.

Non-limiting examples of Linkers that can be used in a protein stabilizing compound of the present invention are exemplified by the compounds drawn herein and the following embodiments.
1. In certain embodiments Linker is:

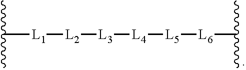

2. The Linker of embodiment 1, wherein L₁ is bond.
3. The Linker of embodiment 1, wherein L₁ is alkyl optionally substituted with 1, 2, 3, or 4 substituents independently selected from R⁴⁴.
4. The Linker of embodiment 1, wherein L₁ is alkene optionally substituted with 1, 2, 3, or 4 substituents independently selected from R⁴⁴.
5. The Linker of embodiment 1, wherein L₁ is alkyne optionally substituted with 1, 2, 3, or 4 substituents independently selected from R⁴⁴.
6. The Linker of embodiment 1, wherein L₁ is haloalkyl optionally substituted with 1, 2, 3, or 4 substituents independently selected from R⁴⁴.
7. The Linker of embodiment 1, wherein L₁ is aryl optionally substituted with 1, 2, 3, or 4 substituents independently selected from R⁴⁴.
8. The Linker of embodiment 1, wherein L₁ is heterocycle optionally substituted with 1, 2, 3, or 4 substituents independently selected from R⁴⁴.
9. The Linker of embodiment 1, wherein L₁ is heteroaryl optionally substituted with 1, 2, 3, or 4 substituents independently selected from R⁴⁴.
10. The Linker of embodiment 1, wherein L₁ is bicycle optionally substituted with 1, 2, 3, or 4 substituents independently selected from R⁴⁴.
11. The Linker of embodiment 1, wherein L₁ is —C(O)—.
12. The Linker of embodiment 1, wherein L₁ is —C(O)O—.
13. The Linker of embodiment 1, wherein L₁ is —OC(O)—.
14. The Linker of embodiment 1, wherein L₁ is —SO₂—.
15. The Linker of embodiment 1, wherein L₁ is —S(O)—.
16. The Linker of embodiment 1, wherein L₁ is —C(S)—.
17. The Linker of embodiment 1, wherein L₁ is —C(O)NR¹¹—.
18. The Linker of embodiment 1, wherein L₁ is —NR¹¹C(O)—.
19. The Linker of embodiment 1, wherein L₁ is —O—.
20. The Linker of embodiment 1, wherein L₁ is —S—.
21. The Linker of embodiment 1, wherein L₁ is —NR¹¹—.
22. The Linker of embodiment 1, wherein L₁ is —P(O)(OR¹¹)O—.
23. The Linker of embodiment 1, wherein L₁ is —P(O)(OR¹¹)—.
24. The Linker of embodiment 1, wherein L₁ is polyethylene glycol.
25. The Linker of embodiment 1, wherein L₁ is lactic acid.
26. The Linker of embodiment 1, wherein L₁ is glycolic acid.
27. The Linker of any one of embodiments 1-26, wherein L₂ is bond.
28. The Linker of any one of embodiments 1-26, wherein L₂ is alkyl optionally substituted with 1, 2, 3, or 4 substituents independently selected from R⁴⁴.
29. The Linker of any one of embodiments 1-26, wherein L₂ is alkene optionally substituted with 1, 2, 3, or 4 substituents independently selected from R⁴⁴.
30. The Linker of any one of embodiments 1-26, wherein L₂ is alkyne optionally substituted with 1, 2, 3, or 4 substituents independently selected from R⁴⁴.
31. The Linker of any one of embodiments 1-26, wherein L₂ is haloalkyl optionally substituted with 1, 2, 3, or 4 substituents independently selected from R⁴⁴.
32. The Linker of any one of embodiments 1-26, wherein L₂ is aryl optionally substituted with 1, 2, 3, or 4 substituents independently selected from R⁴⁴.
33. The Linker of any one of embodiments 1-26, wherein L₂ is heterocycle optionally substituted with 1, 2, 3, or 4 substituents independently selected from R⁴⁴.
34. The Linker of any one of embodiments 1-26, wherein L₂ is heteroaryl optionally substituted with 1, 2, 3, or 4 substituents independently selected from R⁴⁴.
35. The Linker of any one of embodiments 1-26, wherein L₂ is bicycle optionally substituted with 1, 2, 3, or 4 substituents independently selected from R⁴⁴.
36. The Linker of any one of embodiments 1-35, wherein L₃ is bond.
37. The Linker of any one of embodiments 1-35, wherein L₃ is alkyl optionally substituted with 1, 2, 3, or 4 substituents independently selected from R⁴⁴.
38. The Linker of any one of embodiments 1-35, wherein L₃ is alkene optionally substituted with 1, 2, 3, or 4 substituents independently selected from R⁴⁴.
39. The Linker of any one of embodiments 1-35, wherein L₃ is alkyne optionally substituted with 1, 2, 3, or 4 substituents independently selected from R⁴⁴.
40. The Linker of any one of embodiments 1-35, wherein L₃ is haloalkyl optionally substituted with 1, 2, 3, or 4 substituents independently selected from R⁴⁴.
41. The Linker of any one of embodiments 1-35, wherein L₃ is aryl optionally substituted with 1, 2, 3, or 4 substituents independently selected from R⁴⁴.
42. The Linker of any one of embodiments 1-35, wherein L₃ is heterocycle optionally substituted with 1, 2, 3, or 4 substituents independently selected from R⁴⁴.
43. The Linker of any one of embodiments 1-35, wherein L₃ is heteroaryl optionally substituted with 1, 2, 3, or 4 substituents independently selected from R⁴⁴.
44. The Linker of any one of embodiments 1-35, wherein L₃ is bicycle optionally substituted with 1, 2, 3, or 4 substituents independently selected from R⁴⁴.
45. The Linker of any one of embodiments 1-35, wherein L₃ is —C(O)—.
46. The Linker of any one of embodiments 1-35, wherein L₃ is —C(O)O—.
47. The Linker of any one of embodiments 1-35, wherein L₃ is —OC(O)—.
48. The Linker of any one of embodiments 1-35, wherein L₃ is —SO₂—.
49. The Linker of any one of embodiments 1-35, wherein L₃ is —S(O)—.
50. The Linker of any one of embodiments 1-35, wherein L₃ is —C(S)—.
51. The Linker of any one of embodiments 1-35, wherein L₃ is —C(O)NR¹¹—.
52. The Linker of any one of embodiments 1-35, wherein L₃ is —NR¹¹¹C(O)—.
53. The Linker of any one of embodiments 1-35, wherein L₃ is —O—.
54. The Linker of any one of embodiments 1-35, wherein L₃ is —S—.
55. The Linker of any one of embodiments 1-35, wherein L₃ is —NR¹¹—.
56. The Linker of any one of embodiments 1-35, wherein L₃ is —P(O)(OR¹¹)O—.
57. The Linker of any one of embodiments 1-35, wherein L₃ is —P(O)(OR¹¹)—.
58. The Linker of any one of embodiments 1-35, wherein L₃ is polyethylene glycol.
59. The Linker of any one of embodiments 1-35, wherein L₃ is lactic acid.
60. The Linker of any one of embodiments 1-35, wherein L₃ is glycolic acid.
61. The Linker of any one of embodiments 1-60, wherein L₄ is bond.
62. The Linker of any one of embodiments 1-60, wherein L₄ is alkyl optionally substituted with 1, 2, 3, or 4 substituents independently selected from R⁴⁴.
63. The Linker of any one of embodiments 1-60, wherein L₄ is alkene optionally substituted with 1, 2, 3, or 4 substituents independently selected from R⁴⁴.
64. The Linker of any one of embodiments 1-60, wherein L₄ is alkyne optionally substituted with 1, 2, 3, or 4 substituents independently selected from R⁴⁴.
65. The Linker of any one of embodiments 1-60, wherein L₄ is haloalkyl optionally substituted with 1, 2, 3, or 4 substituents independently selected from R⁴⁴.
66. The Linker of any one of embodiments 1-60, wherein L₄ is aryl optionally substituted with 1, 2, 3, or 4 substituents independently selected from R⁴⁴.
67. The Linker of any one of embodiments 1-60, wherein L₄ is heterocycle optionally substituted with 1, 2, 3, or 4 substituents independently selected from R⁴⁴.
68. The Linker of any one of embodiments 1-60, wherein L₄ is heteroaryl optionally substituted with 1, 2, 3, or 4 substituents independently selected from R⁴⁴.
69. The Linker of any one of embodiments 1-60, wherein L₄ is bicycle optionally substituted with 1, 2, 3, or 4 substituents independently selected from R⁴⁴.
70. The Linker of any one of embodiments 1-69, wherein L₅ is bond.
71. The Linker of any one of embodiments 1-69, wherein L₅ is alkyl optionally substituted with 1, 2, 3, or 4 substituents independently selected from R⁴⁴.
72. The Linker of any one of embodiments 1-69, wherein L₅ is alkene optionally substituted with 1, 2, 3, or 4 substituents independently selected from R⁴⁴.
73. The Linker of any one of embodiments 1-69, wherein L₅ is alkyne optionally substituted with 1, 2, 3, or 4 substituents independently selected from R⁴⁴.
74. The Linker of any one of embodiments 1-69, wherein L₅ is haloalkyl optionally substituted with 1, 2, 3, or 4 substituents independently selected from R⁴⁴.
75. The Linker of any one of embodiments 1-69, wherein L₅ is aryl optionally substituted with 1, 2, 3, or 4 substituents independently selected from R⁴⁴.
76. The Linker of any one of embodiments 1-69, wherein L₅ is heterocycle optionally substituted with 1, 2, 3, or 4 substituents independently selected from R⁴⁴.
77. The Linker of any one of embodiments 1-69, wherein L₅ is heteroaryl optionally substituted with 1, 2, 3, or 4 substituents independently selected from R⁴⁴.
78. The Linker of any one of embodiments 1-69, wherein L₅ is bicycle optionally substituted with 1, 2, 3, or 4 substituents independently selected from R⁴⁴.
79. The Linker of any one of embodiments 1-78, wherein L₆ is bond.
80. The Linker of any one of embodiments 1-78, wherein L₆ is alkyl optionally substituted with 1, 2, 3, or 4 substituents independently selected from R⁴⁴.
81. The Linker of any one of embodiments 1-78, wherein L₆ is alkene optionally substituted with 1, 2, 3, or 4 substituents independently selected from R⁴⁴.
82. The Linker of any one of embodiments 1-78, wherein L₆ is alkyne optionally substituted with 1, 2, 3, or 4 substituents independently selected from R⁴⁴.
83. The Linker of any one of embodiments 1-78, wherein L₆ is haloalkyl optionally substituted with 1, 2, 3, or 4 substituents independently selected from R⁴⁴.
84. The Linker of any one of embodiments 1-78, wherein L₆ is aryl optionally substituted with 1, 2, 3, or 4 substituents independently selected from R⁴⁴.
85. The Linker of any one of embodiments 1-78, wherein L₆ is heterocycle optionally substituted with 1, 2, 3, or 4 substituents independently selected from R⁴⁴.
86. The Linker of any one of embodiments 1-78, wherein L₆ is heteroaryl optionally substituted with 1, 2, 3, or 4 substituents independently selected from R⁴⁴.
87. The Linker of any one of embodiments 1-78, wherein L₆ is bicycle optionally substituted with 1, 2, 3, or 4 substituents independently selected from R⁴⁴.
88. The Linker of any one of embodiments 1-87, wherein L₁ is bound to USP7 Targeting Ligand.
89. The Linker of any one of embodiments 1-87, wherein L₁ is bound to Ubiquitinated Protein Targeting Ligand.
90. The Linker of any one of embodiments 1-89, wherein R⁴⁴ is independently selected at each instance from alkyl, halogen, and haloalkyl.
91. The Linker of any one of embodiments 1-89, wherein R⁴⁴ is alkyl optionally substituted as allowed by valence with 1, 2, 3, or 4 substituents selected from R⁴⁵.
92. The Linker of any one of embodiments 1-89, wherein R⁴⁴ is aryl optionally substituted as allowed by valence with 1, 2, 3, or 4 substituents selected from R⁴⁵.
93. The Linker of any one of embodiments 1-89, wherein R⁴⁴ is heterocycle optionally substituted as allowed by valence with 1, 2, 3, or 4 substituents selected from R⁴⁵.
94. The Linker of any one of embodiments 1-89, wherein R⁴⁴ is heteroaryl optionally substituted as allowed by valence with 1, 2, 3, or 4 substituents selected from R⁴⁵.
95. The Linker of any one of embodiments 1-89, wherein R⁴⁴ is amino optionally substituted as allowed by valence with 1, 2, 3, or 4 substituents selected from R⁴⁵.
96. The Linker of any one of embodiments 1-89, wherein R⁴⁴ is hydroxyl optionally substituted as allowed by valence with 1, 2, 3, or 4 substituents selected from R⁴⁵.
97. The Linker of any one of embodiments 1-89, wherein R⁴⁴ is alkoxy optionally substituted as allowed by valence with 1, 2, 3, or 4 substituents selected from R⁴⁵.
98. The Linker of any one of embodiments 1-89, wherein R⁴⁴ is cyano.
99. The Linker of any one of embodiments 1-89, wherein R⁴⁴ is nitro.
100. The Linker of any one of embodiments 1-89, wherein R⁴⁴ is —OC(O)R⁴⁰.
101. The Linker of any one of embodiments 1-89, wherein R⁴⁴ is —NR¹¹C(O)R⁴⁰.
102. The Linker of any one of embodiments 1-89, wherein R⁴⁴ is —C(O)R⁴⁰.
103. The Linker of any one of embodiments 1-89, wherein R⁴⁴ is —OP(O)(R⁴⁰)₂.
104. The Linker of any one of embodiments 1-89, wherein R⁴⁴ is —P(O)(R⁴⁰)₂.
105. The Linker of any one of embodiments 1-89, wherein R⁴⁴ is —NR¹¹P(O)(R⁴⁰)₂.
106. The Linker of any one of embodiments 1-89, wherein R⁴⁴ is —SR¹¹¹.
107. The Linker of any one of embodiments 1-89, wherein R⁴⁴ is —OR¹¹.
108. The Linker of any one of embodiments 1-89, wherein R⁴⁴ is —S(O)R⁴⁰.
109. The Linker of any one of embodiments 1-89, wherein R⁴⁴ is —S(O)₂R⁴⁰.
110. The Linker of any one of embodiments 1-89, wherein R⁴⁴ is —N(alkyl)C(O)R⁴⁰.
111. The Linker of any one of embodiments 90-97, wherein R⁴⁵ is independently selected from halogen, alkyl, and haloalkyl.
112. The Linker of any one of embodiments 90-97, wherein R⁴⁵ is independently selected from amino, hydroxyl, alkoxy, —NHalkyl, —N(alkyl)₂, —OC(O)alkyl, —NHC(O)alkyl, and —N(alkyl)C(O)alkyl.

In certain embodiments, Linker is selected from:

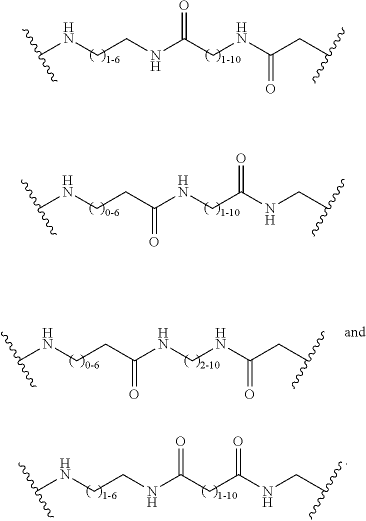 [SMILES: *CC(=O)NCC(=O)NCCN* ; *CNC(=O)CNC(=O)CCN* ; *CC(=O)NCNC(=O)CCN* ; *CNC(=O)CC(=O)NCCN*]

In certain embodiments, Linker is selected from:

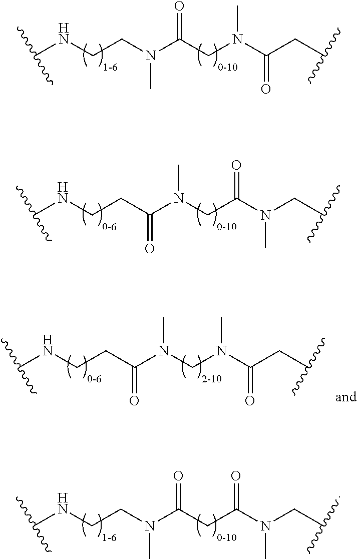

In certain embodiments, Linker is selected from:

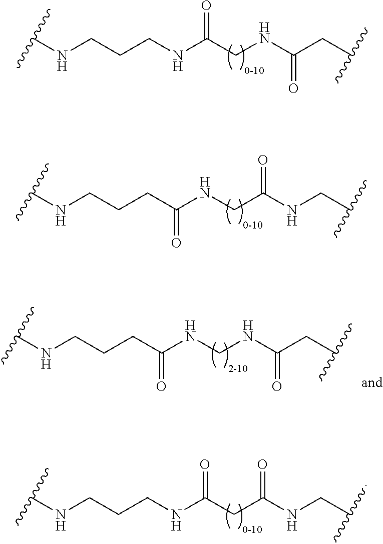

In certain embodiments, Linker is selected from:

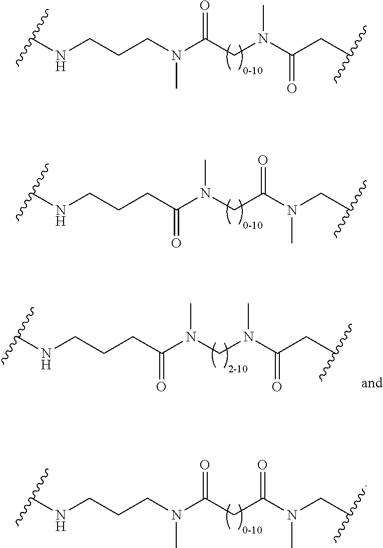

In certain embodiments, Linker is selected from:

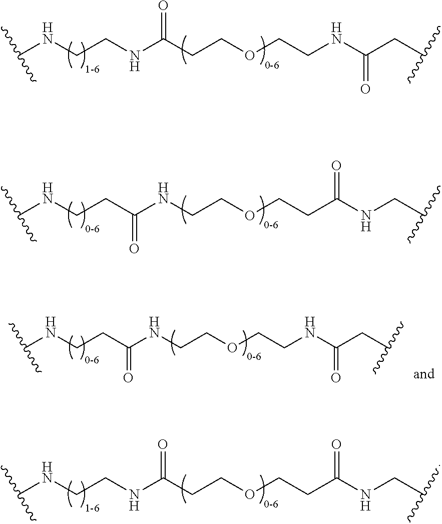

In certain embodiments, Linker is selected from:

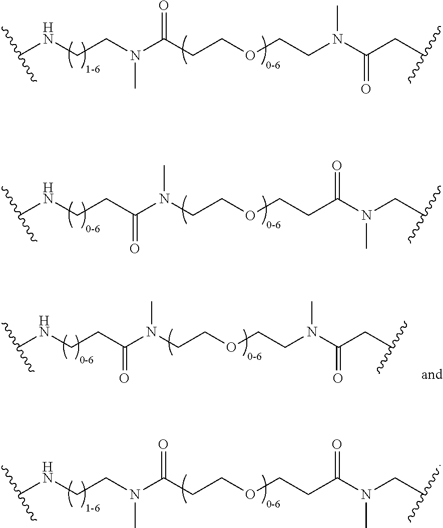

In certain embodiments, Linker is selected from:

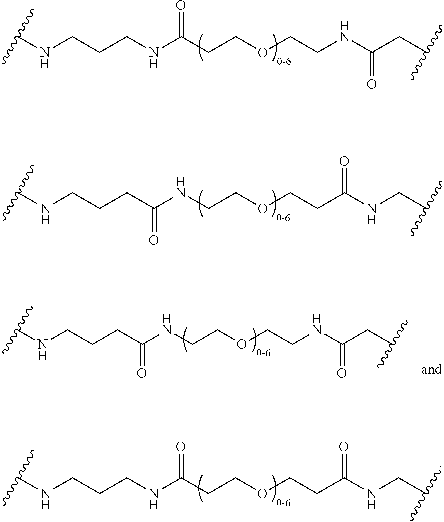

In certain embodiments, Linker is selected from:

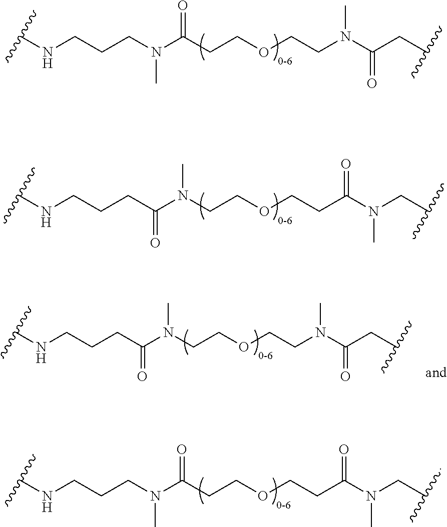

In certain embodiments, Linker is selected from:

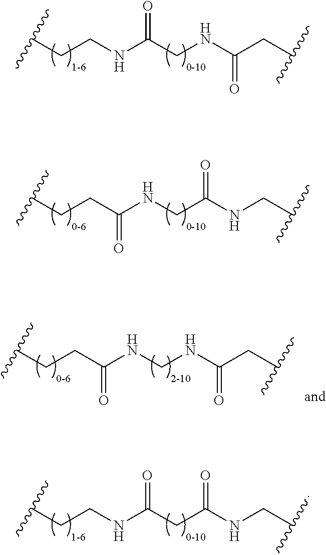

In certain embodiments, Linker is selected from:

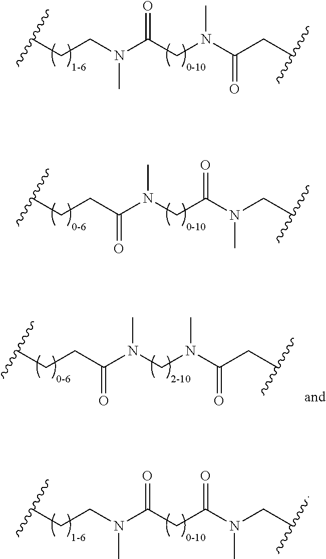

In certain embodiments, Linker is selected from:

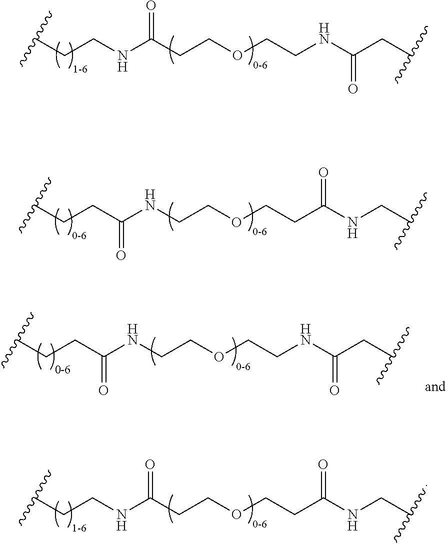

In certain embodiments, Linker is selected from:

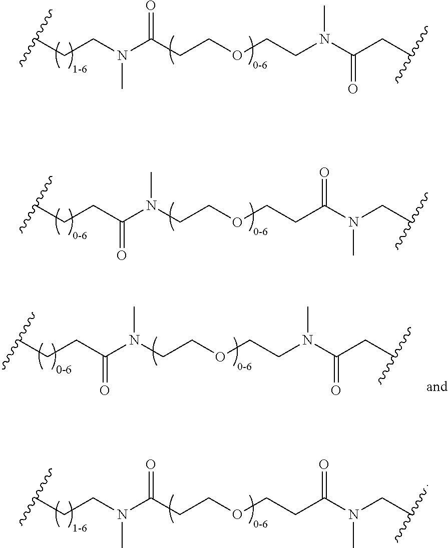

In certain embodiments, Linker is selected from:

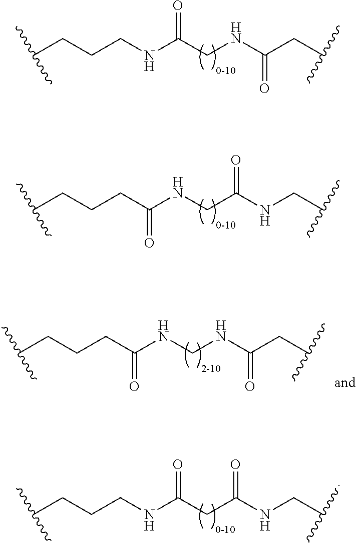

In certain embodiments, Linker is selected from:

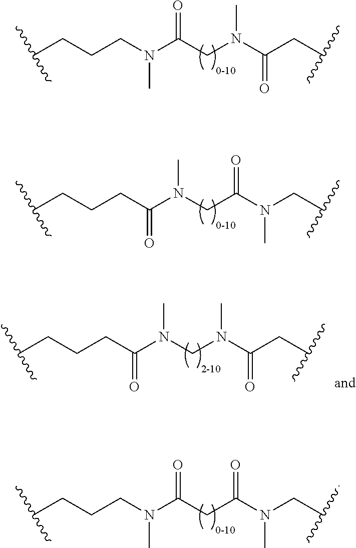

In certain embodiments, Linker is selected from:

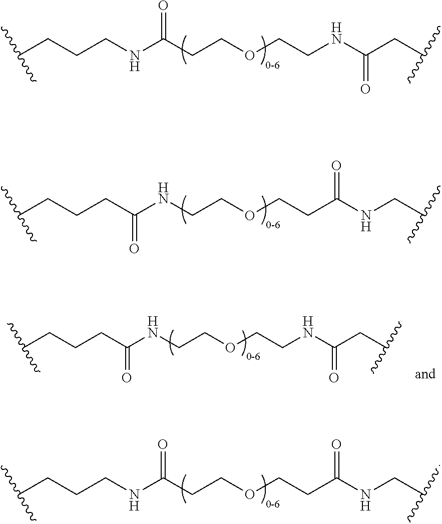

In certain embodiments, Linker is selected from:

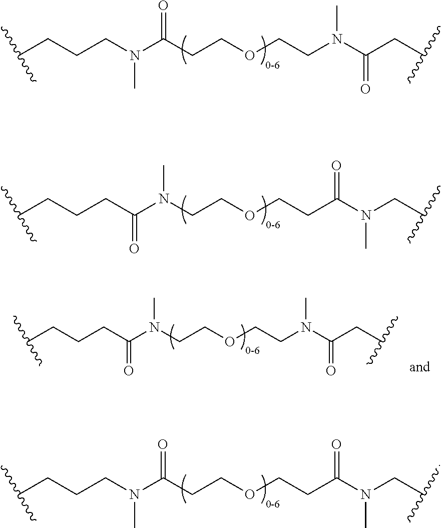

In certain embodiments, Linker is selected from:

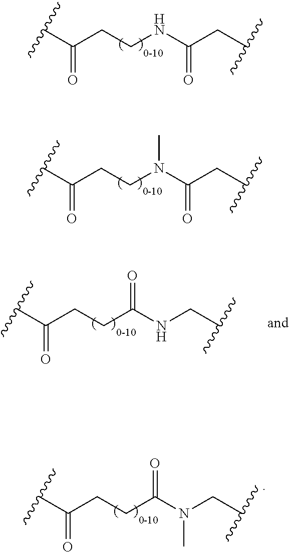

In certain embodiments, Linker is selected from:

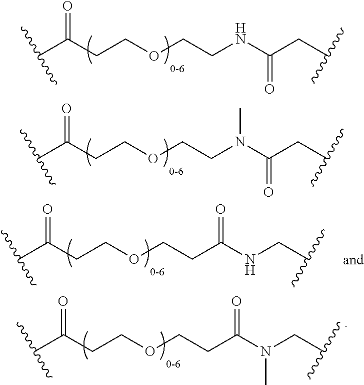

In certain embodiments, Linker is selected from:

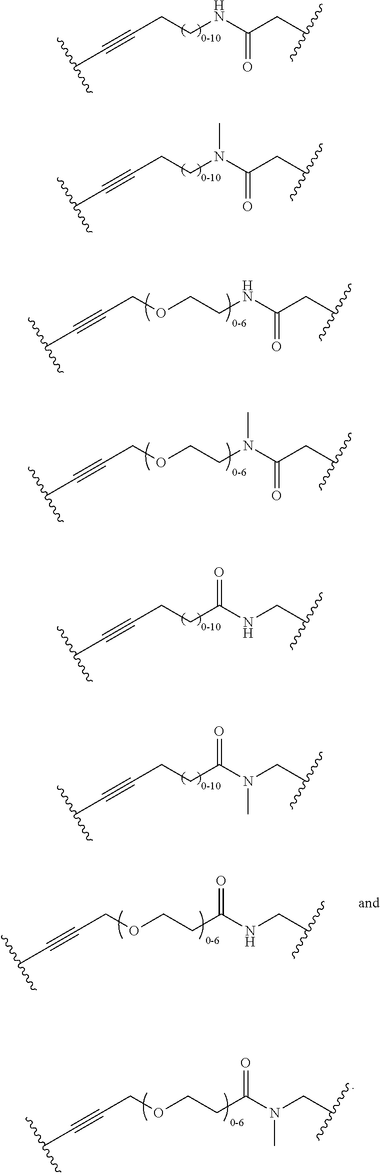

In certain embodiments, Linker is selected from:

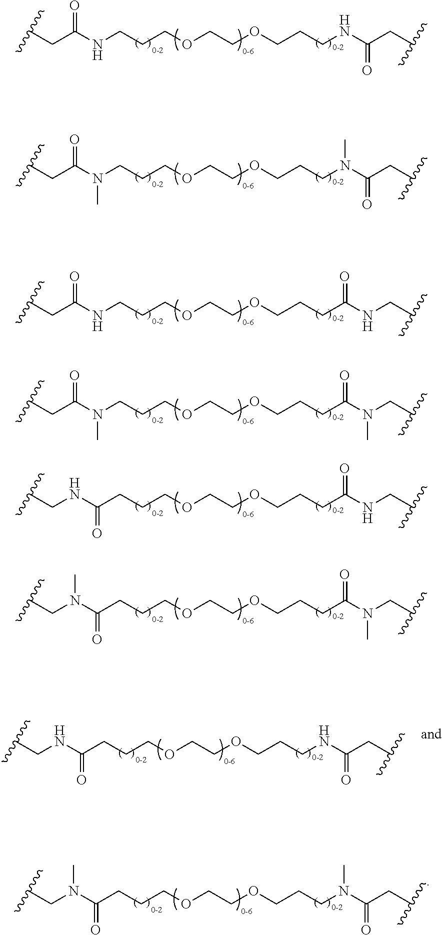

In certain embodiments, Linker is selected from:

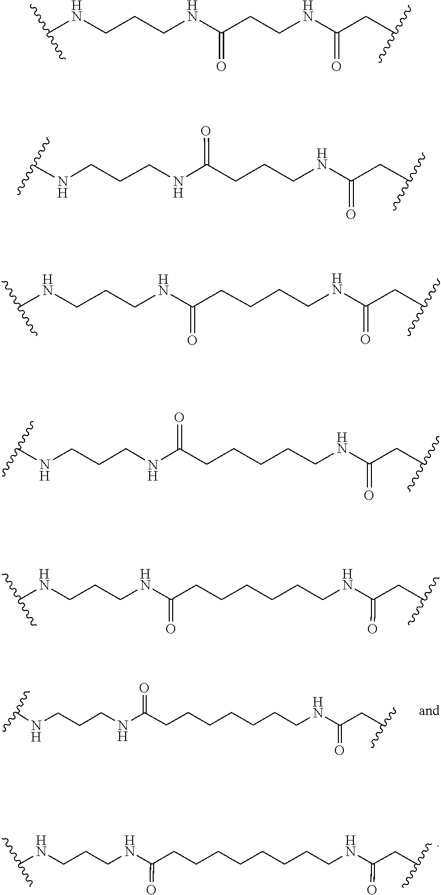

In certain embodiments, Linker is selected from:

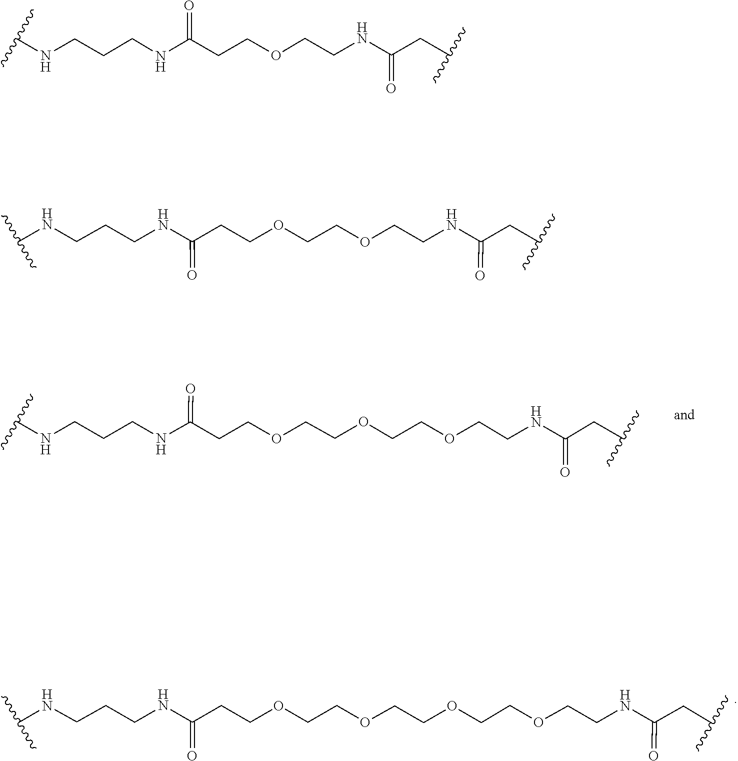 [SMILES: *CC(=O)NCCOCCC(=O)NCCCN* ; *CC(=O)NCCOCCOCCC(=O)NCCCN* ; *CC(=O)NCCOCCOCCOCCC(=O)NCCCN* ; *CC(=O)NCCOCCOCCOCCOCCC(=O)NCCCN*]

In certain embodiments, Linker is selected from:

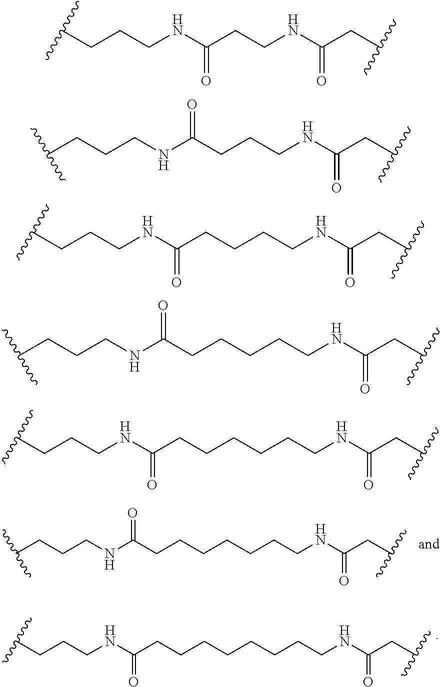

In certain embodiments, Linker is selected from:

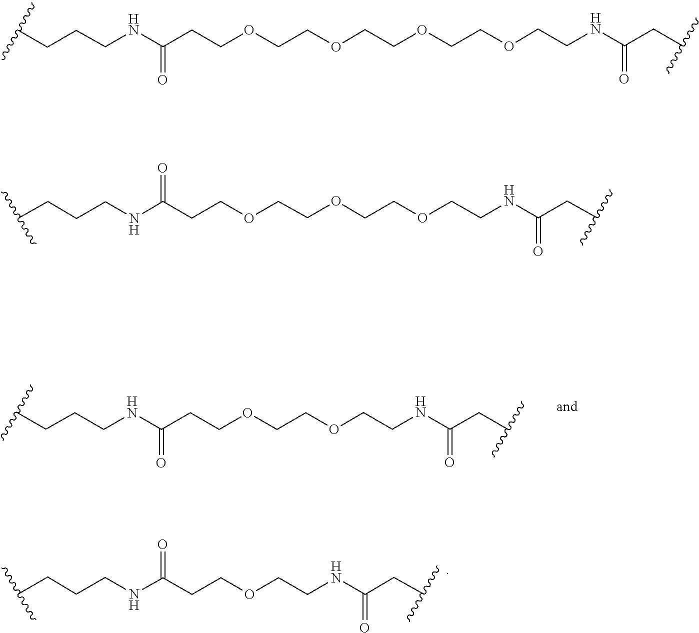 [SMILES: *CCCNC(=O)CCOCCOCCOCCOCCNC(=O)C* ; *CCCNC(=O)CCOCCOCCOCCNC(=O)C* ; *CCCNC(=O)CCOCCOCCNC(=O)C* ; *CCCNC(=O)CCOCCNC(=O)C*]

In certain embodiments, Linker is selected from:

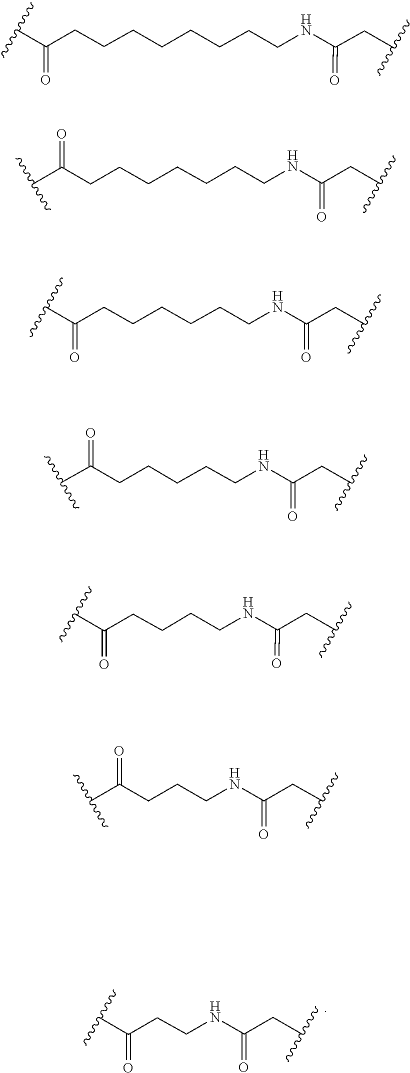

In certain embodiments, Linker is selected from:

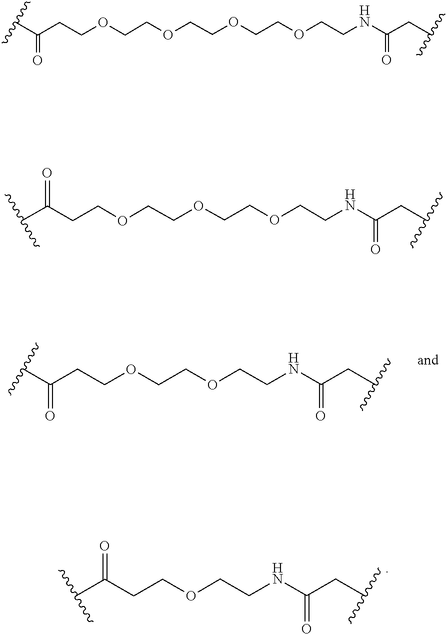 [SMILES: *CC(=O)NCCOCCOCCOCCOCCC(*)=O ; *CC(=O)NCCOCCOCCOCCC(*)=O ; *CC(=O)NCCOCCOCCC(*)=O ; *CC(=O)NCCOCCC(*)=O]

In certain embodiments, Linker is selected from:

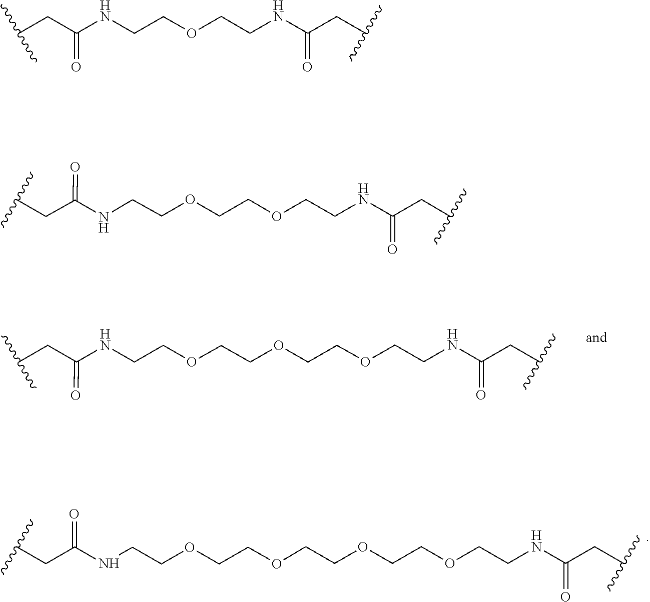 [SMILES: *CC(=O)NCCOCCNC(=O)C* ; *CC(=O)NCCOCCOCCNC(=O)C* ; *CC(=O)NCCOCCOCCOCCNC(=O)C* ; *CC(=O)NCCOCCOCCOCCOCCNC(=O)C*]

In certain embodiments, Linker is selected from:

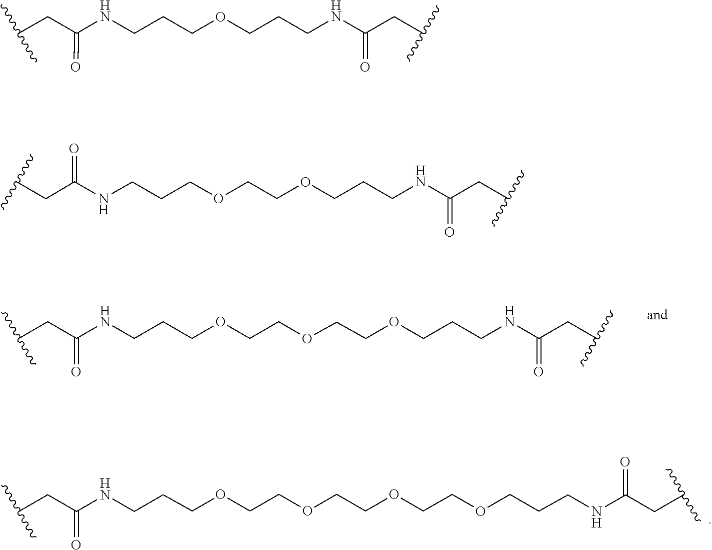 [SMILES: *CC(=O)NCCCOCCCNC(=O)C* ; *CC(=O)NCCCOCCOCCCNC(=O)C* ; *CC(=O)NCCCOCCOCCOCCCNC(=O)C* ; *CC(=O)NCCCOCCOCCOCCOCCCNC(=O)C*]

In certain embodiments Linker is selected from

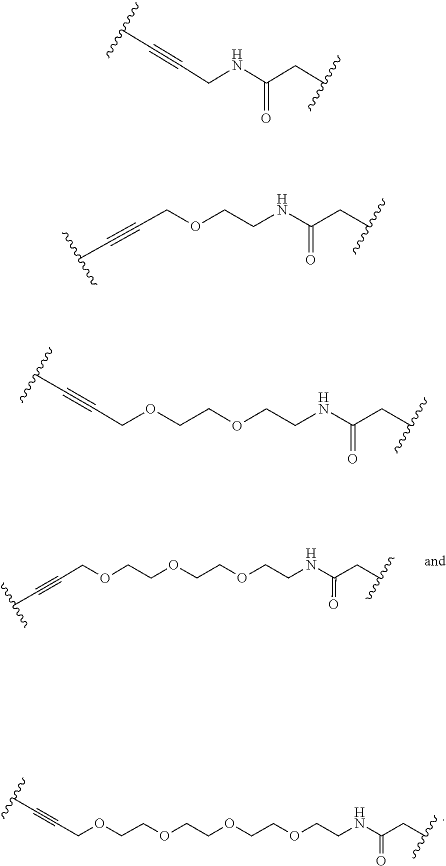

In certain embodiments Linker is selected from:

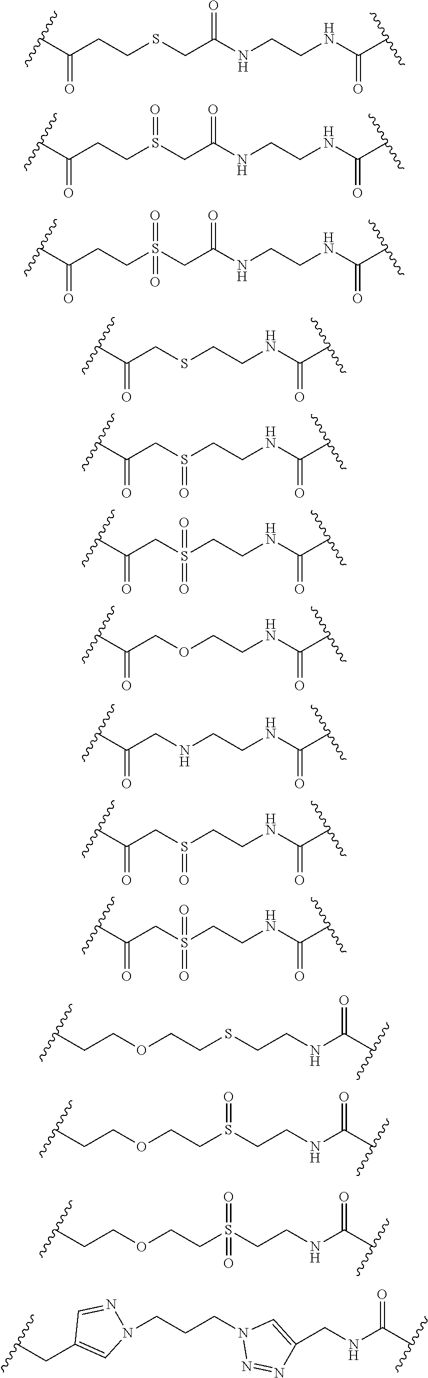

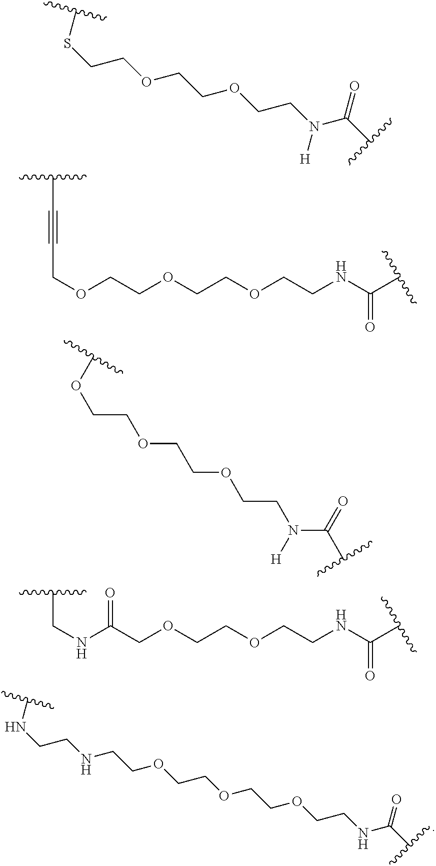 [SMILES: *SCCOCCOCCNC(*)=O ; *C#CCOCCOCCOCCNC(*)=O ; *OCCOCCOCCNC(*)=O ; *CNC(=O)COCCOCCNC(*)=O ; *NCCNCCOCCOCCOCCNC(*)=O]

In certain embodiments, Linker, Linker-A, and/or Linker-B is selected from:

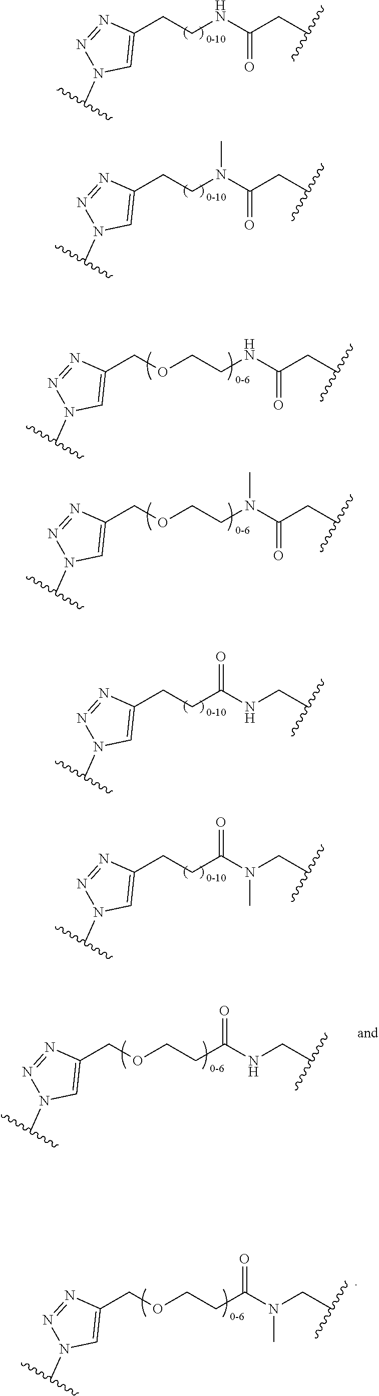

In certain embodiments, Linker, Linker-A, and/or Linker-B is selected from:

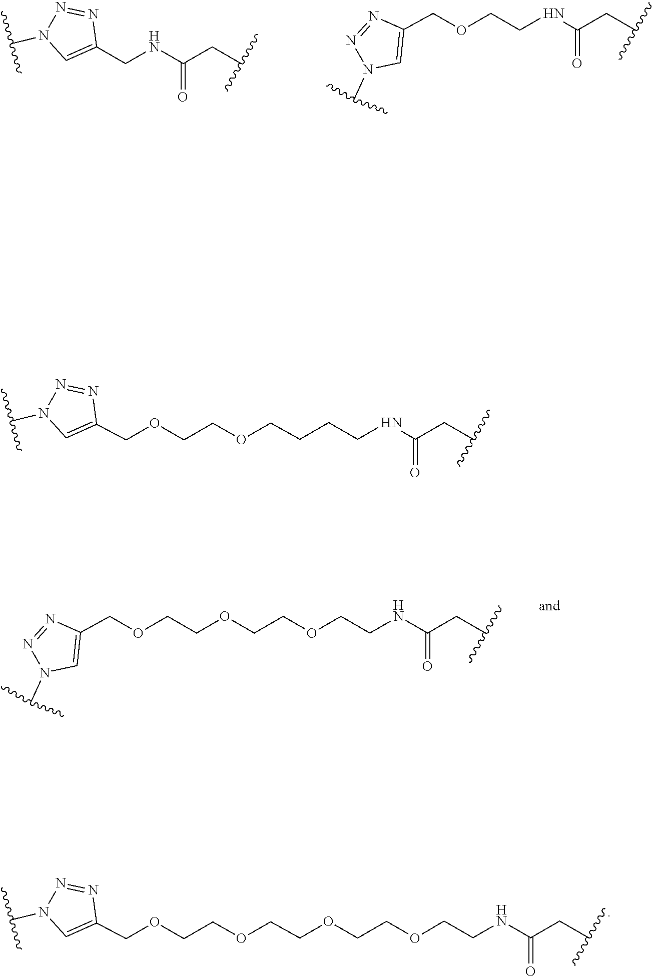

In certain embodiments Linker Linker-A and/or Linker-B is selected from:

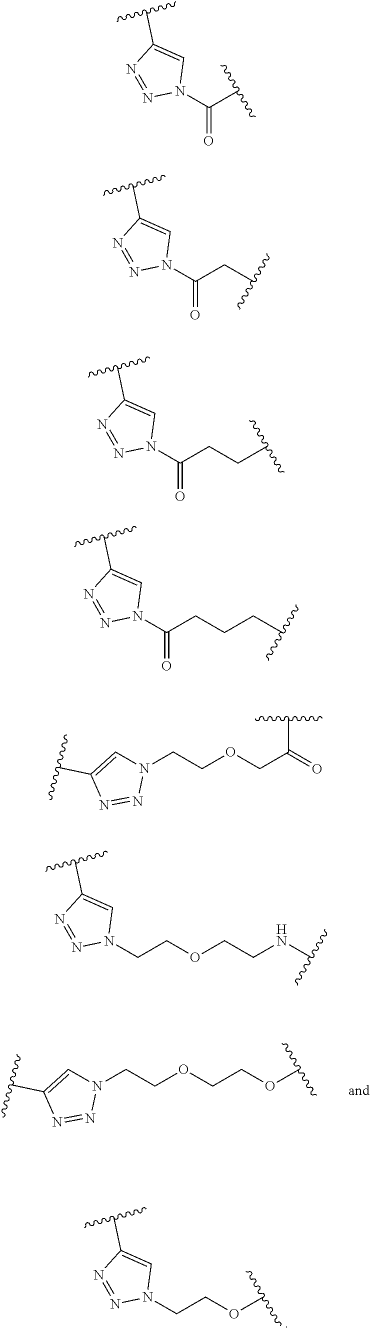

In certain embodiments, Linker-A is selected from:

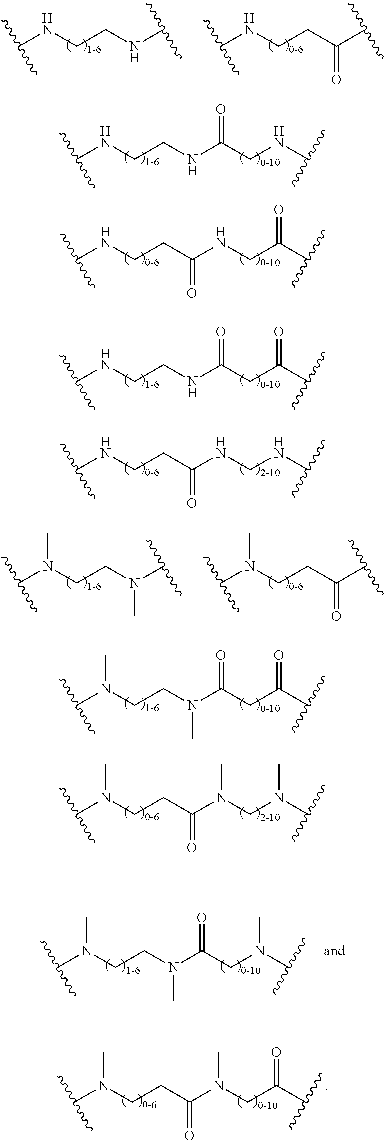

In certain embodiments, Linker-A is selected from:

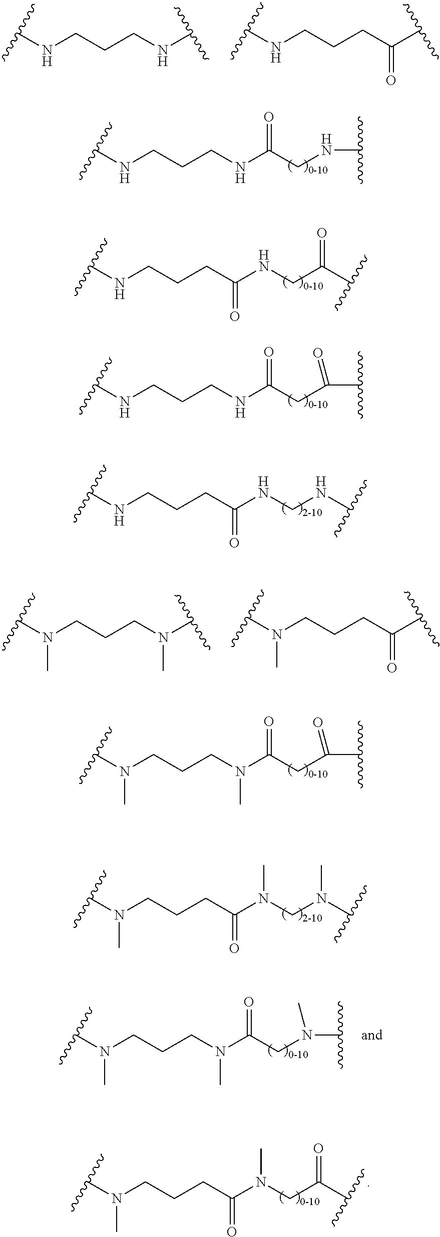

In certain embodiments, Linker-A is selected from:

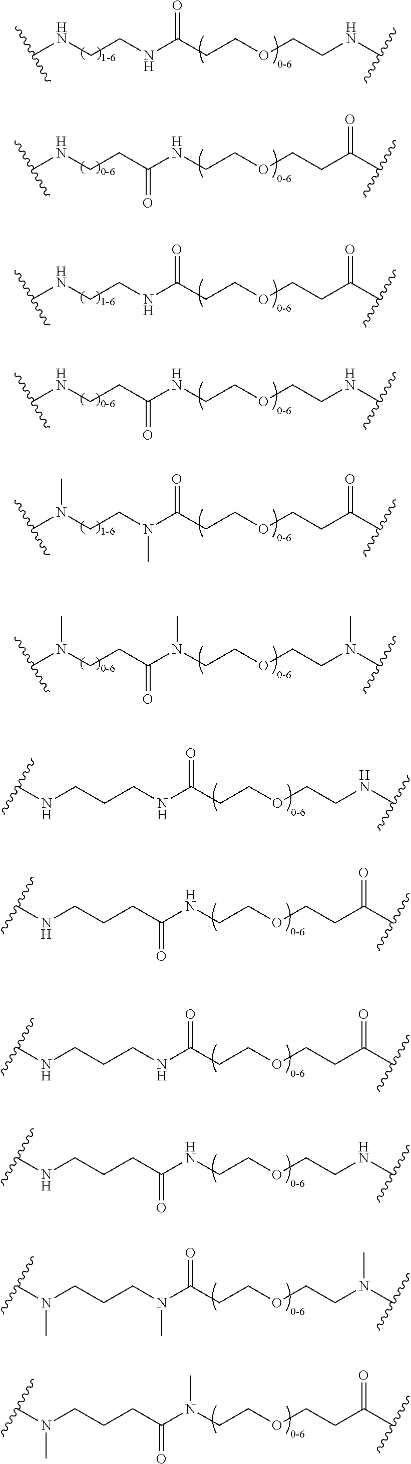

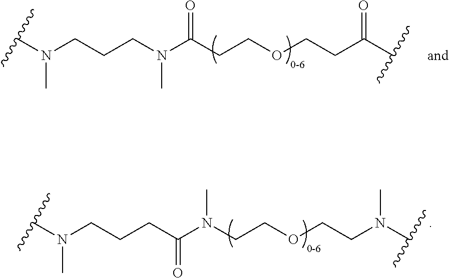

In certain embodiments, Linker-A is selected from:

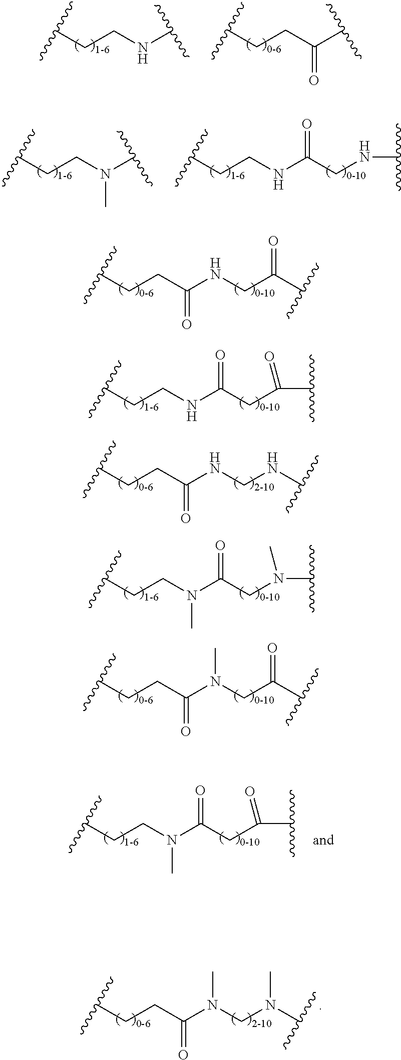

In certain embodiments, Linker-A is selected from:

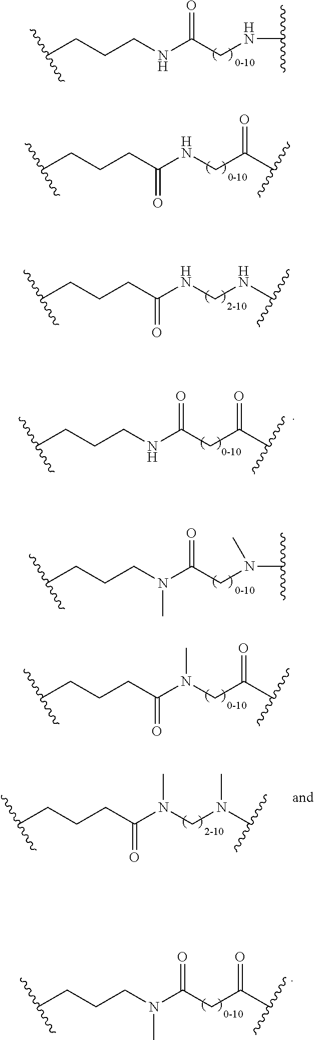

In certain embodiments, Linker-A is selected from:

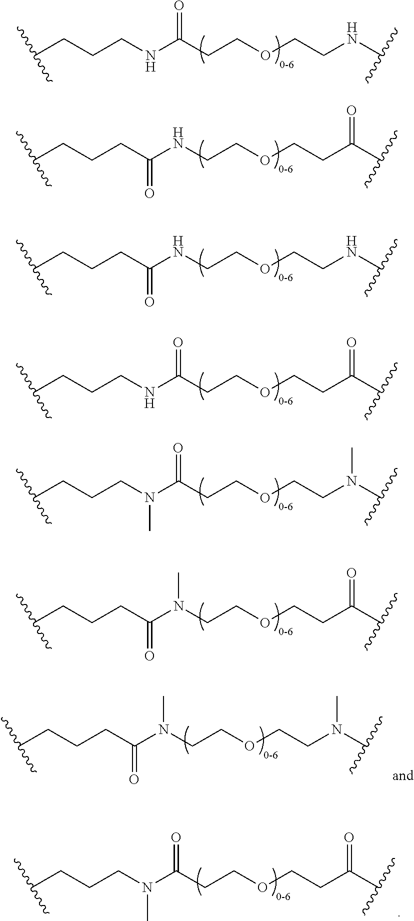

In certain embodiments, Linker-A is selected from:

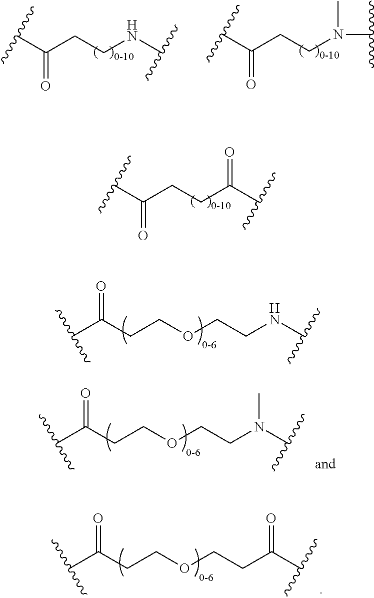

In certain embodiments, Linker-A is selected from:

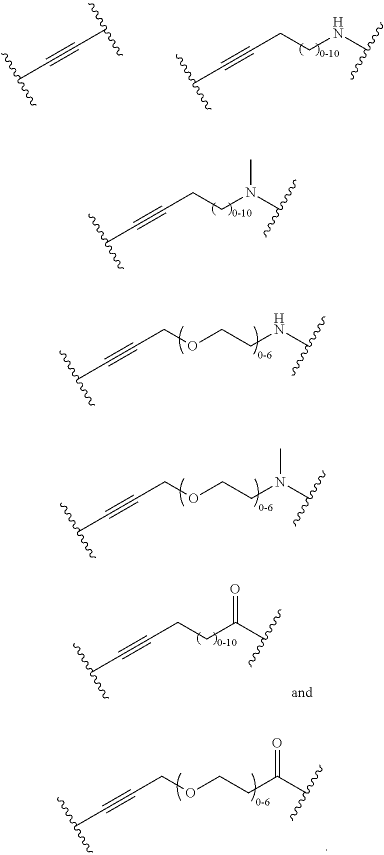

In certain embodiments, Linker-A is selected from:

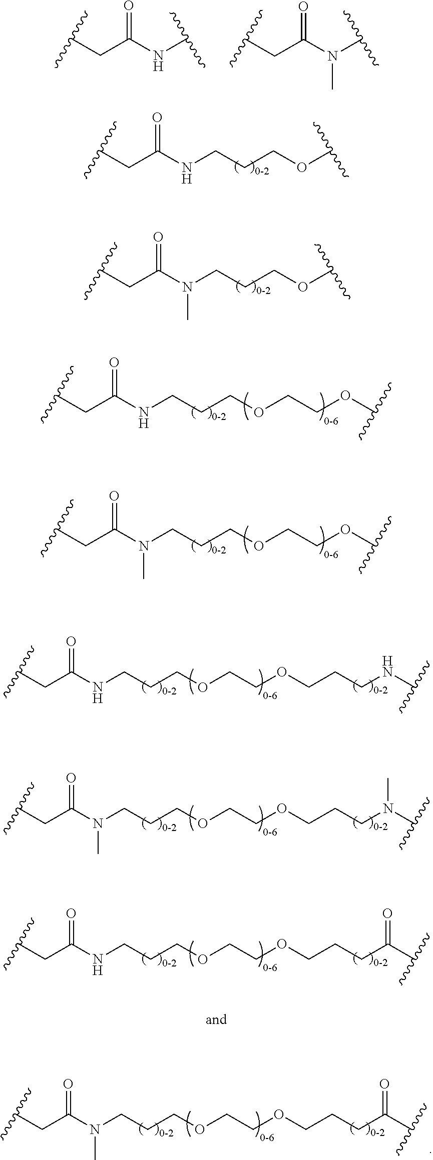

In certain embodiments, Linker-A is selected from:

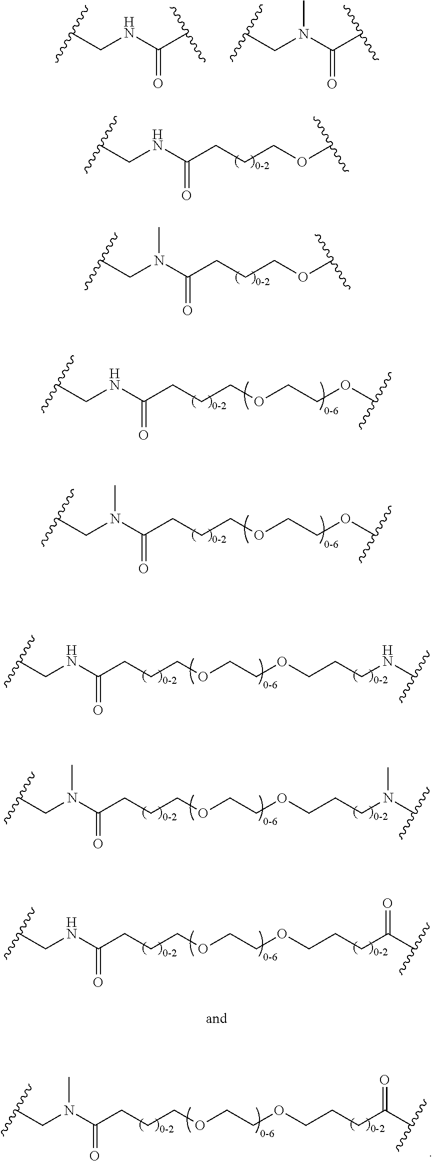

In certain embodiments, Linker-A is selected from:

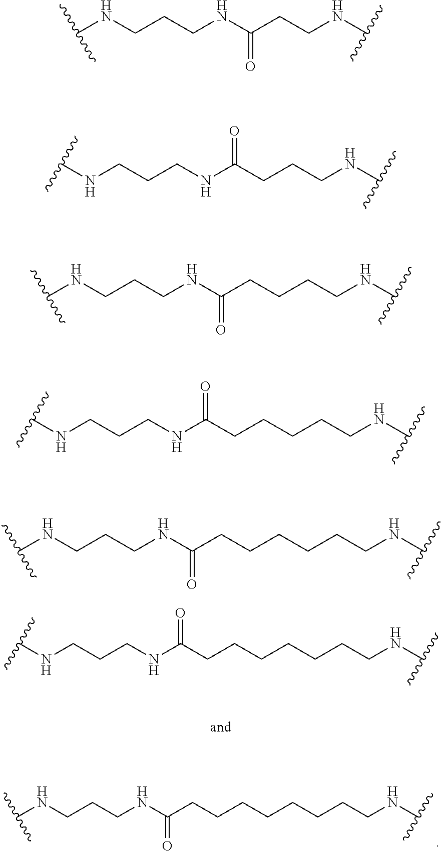

In certain embodiments, Linker-A is selected from:

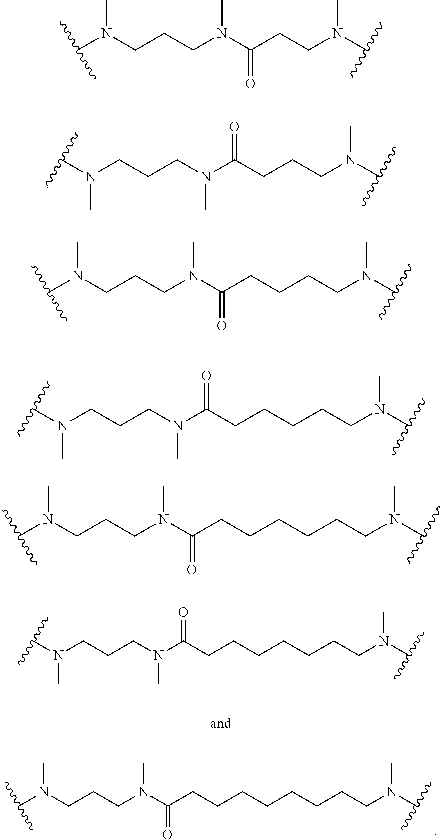

In certain embodiments, Linker-A is selected from:

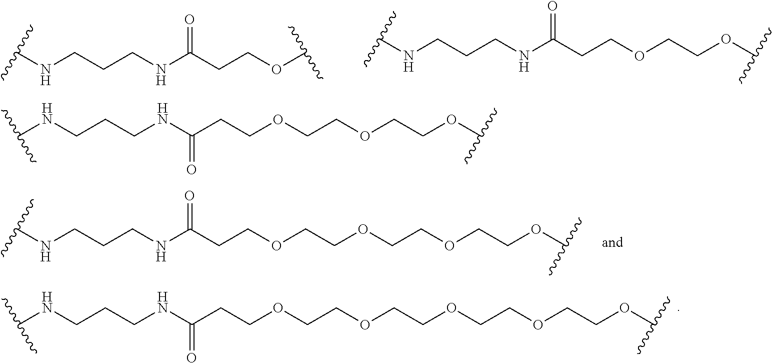

In certain embodiments, Linker-A is selected from:

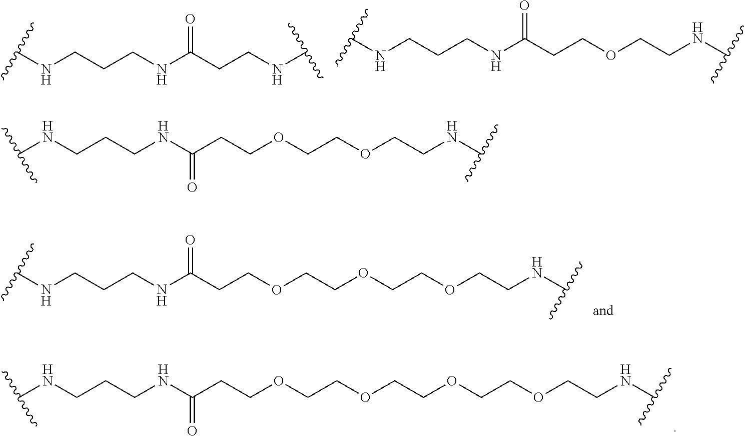 [SMILES: *NCCCNC(=O)CCN* ; *NCCCNC(=O)CCOCCN* ; *NCCCNC(=O)CCOCCOCCN* ; *NCCCNC(=O)CCOCCOCCOCCN* ; *NCCCNC(=O)CCOCCOCCOCCOCCN*]

In certain embodiments, Linker-A is selected from:

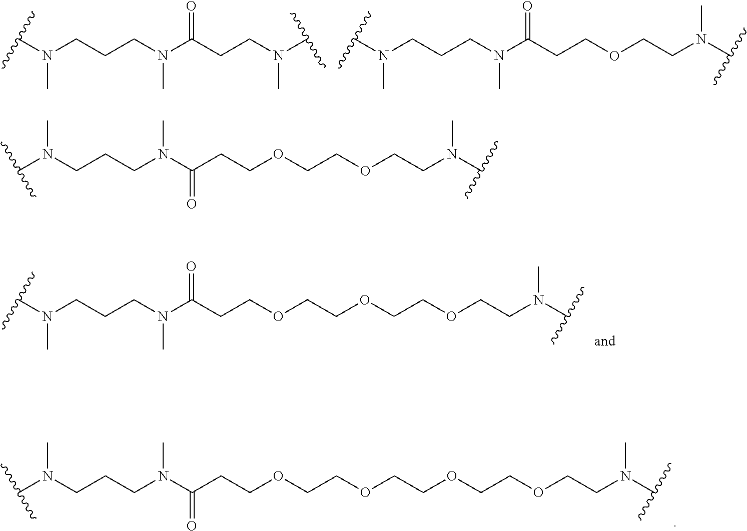

In certain embodiments, Linker-A is selected from:

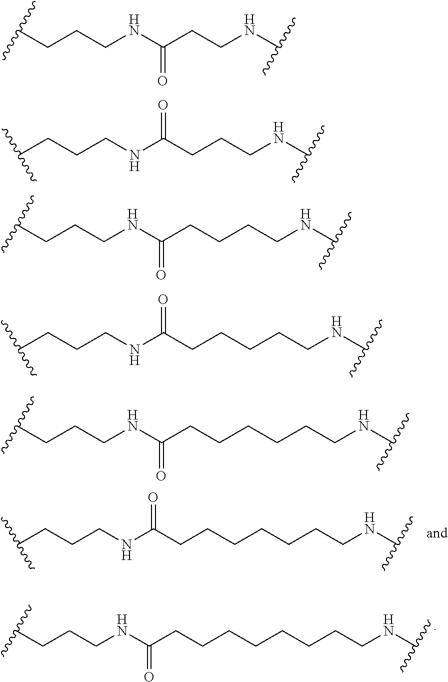

In certain embodiments, Linker-A is selected from:

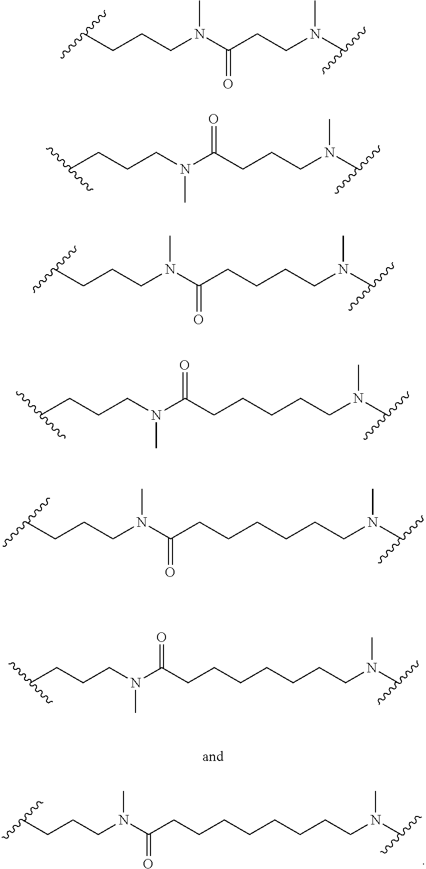

In certain embodiments, Linker-A is selected from:

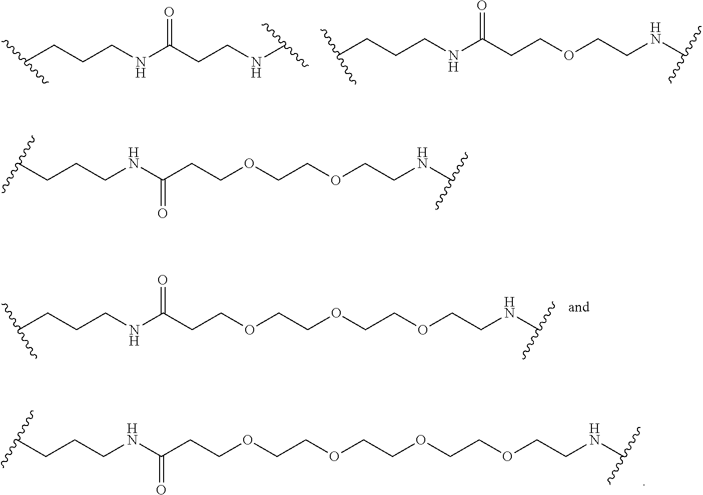 [SMILES: *CCCNC(=O)CCN* ; *CCCNC(=O)CCOCCN* ; *CCCNC(=O)CCOCCOCCN* ; *CCCNC(=O)CCOCCOCCOCCN* ; *CCCNC(=O)CCOCCOCCOCCOCCN*]

In certain embodiments, Linker-A is selected from:

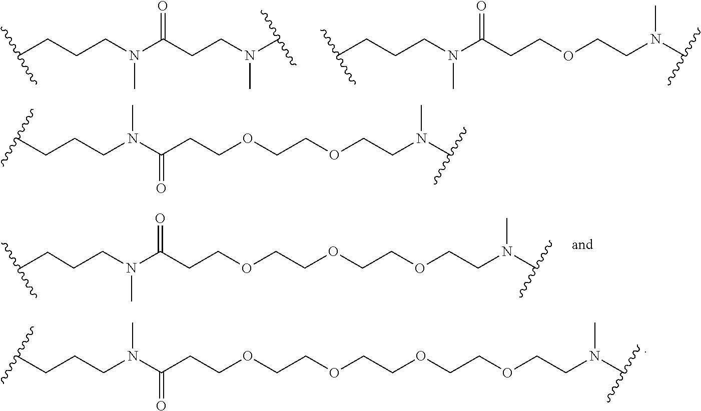

In certain embodiments, Linker-A is selected from:

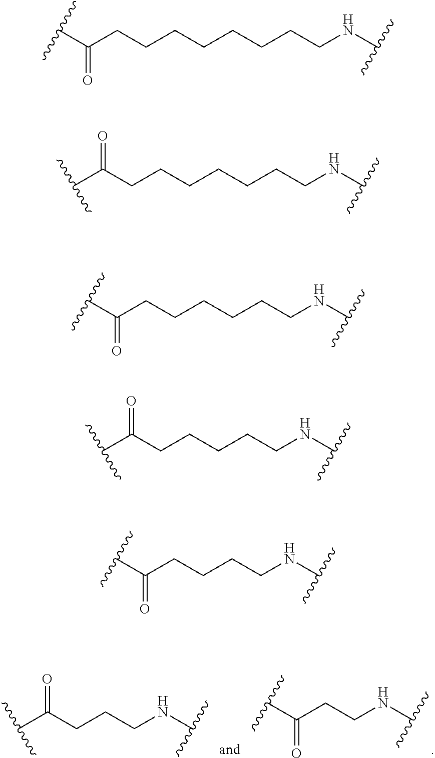

In certain embodiments, Linker-A is selected from:

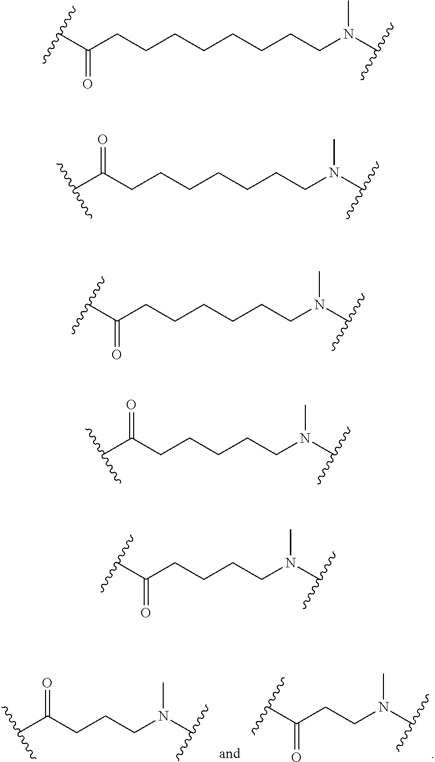

In certain embodiments, Linker-A is selected from:

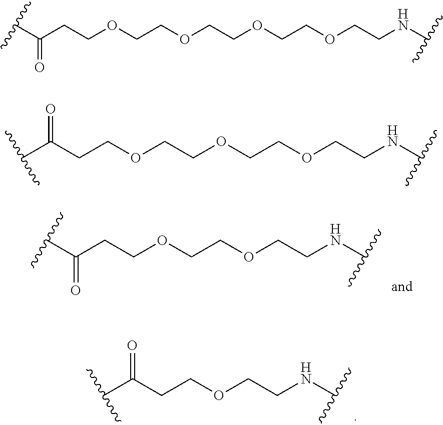

In certain embodiments, Linker-A is selected from:

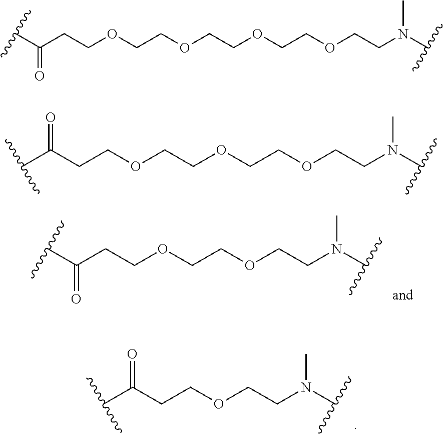

In certain embodiments, Linker-A is selected from:

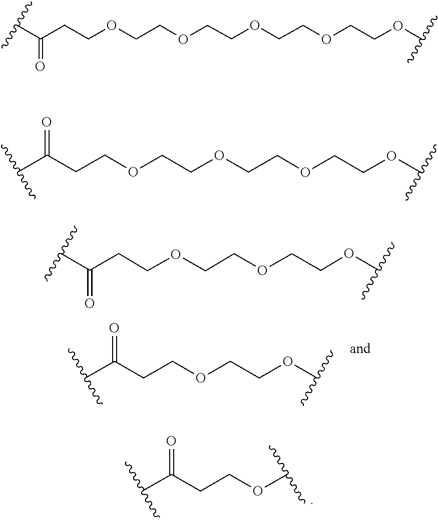

In certain embodiments, Linker-A is selected from:

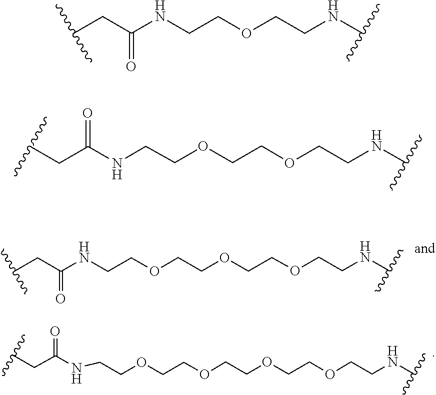

In certain embodiments, Linker-A is selected from:

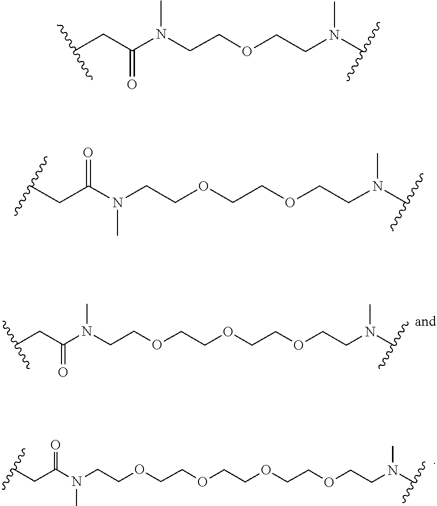 [SMILES: *CC(=O)N(C)CCOCCN(*)C ; *CC(=O)N(C)CCOCCOCCN(*)C ; *CC(=O)N(C)CCOCCOCCOCCN(*)C ; *CC(=O)N(C)CCOCCOCCOCCOCCN(*)C]

In certain embodiments, Linker-A is selected from:

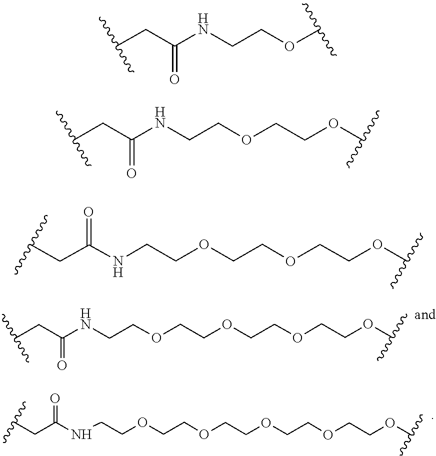

In certain embodiments, Linker-A is selected from:

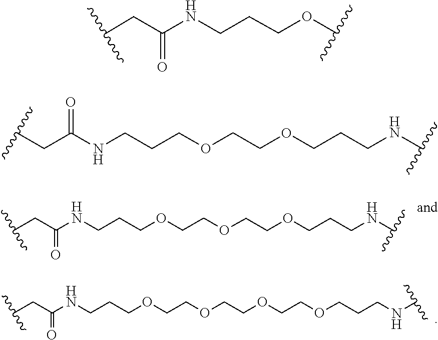 [SMILES: *CC(=O)NCCCO* ; *CC(=O)NCCCOCCOCCCN* ; *CC(=O)NCCCOCCOCCOCCCN* ; *CC(=O)NCCCOCCOCCOCCOCCCN*]

In certain embodiments, Linker-A is selected from:

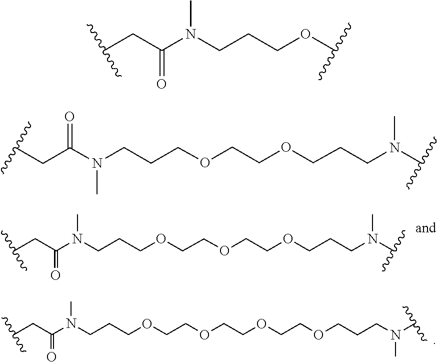

In certain embodiments, Linker-A is selected from:

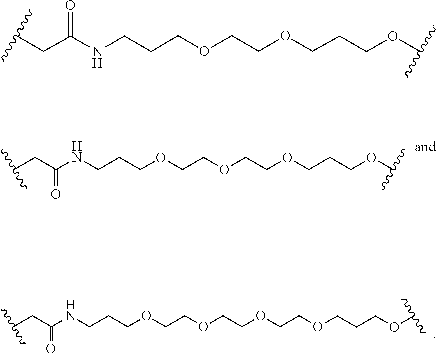 [SMILES: *CC(=O)NCCCOCCOCCCO* ; *CC(=O)NCCCOCCOCCOCCCO* ; *CC(=O)NCCCOCCOCCOCCOCCCO*]

In certain embodiments, Linker-A is selected from:

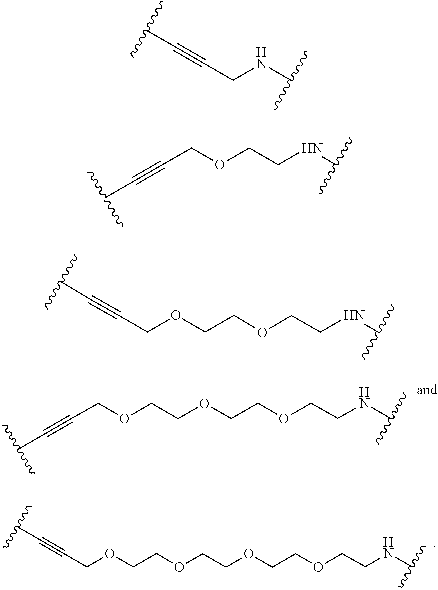 [SMILES: *C#CCN* ; *C#CCOCCN* ; *C#CCOCCOCCN* ; *C#CCOCCOCCOCCN* ; *C#CCOCCOCCOCCOCCN*]

In certain embodiments, Linker-A is selected from:

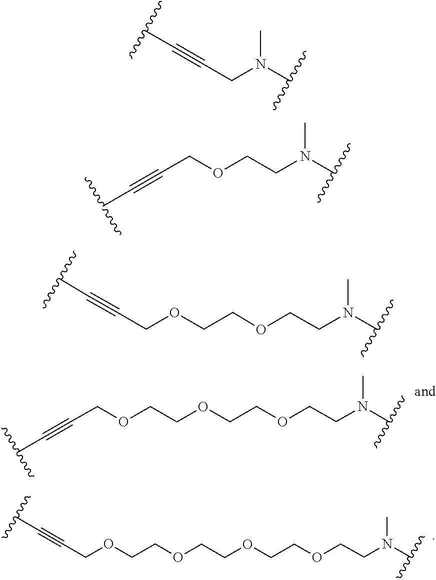

In certain embodiments Linker-A is selected from:

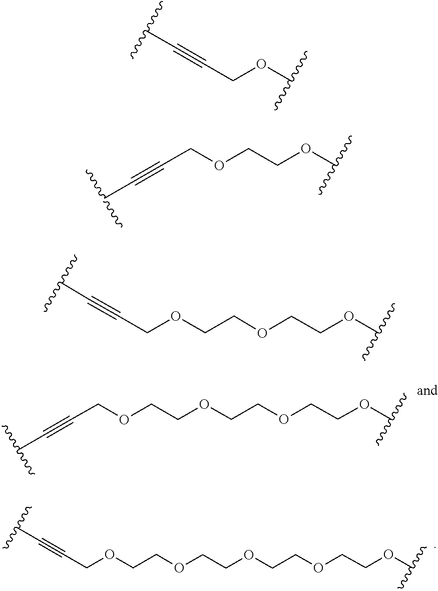 [SMILES: *C#CCO* ; *C#CCOCCO* ; *C#CCOCCOCCO* ; *C#CCOCCOCCOCCO* ; *C#CCOCCOCCOCCOCCO*]

In certain embodiments, Linker-B is selected from:

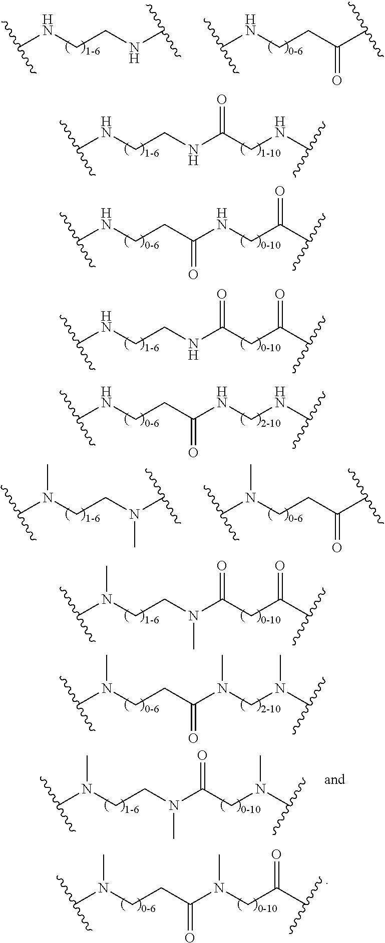

In certain embodiments Linker-B is selected from:

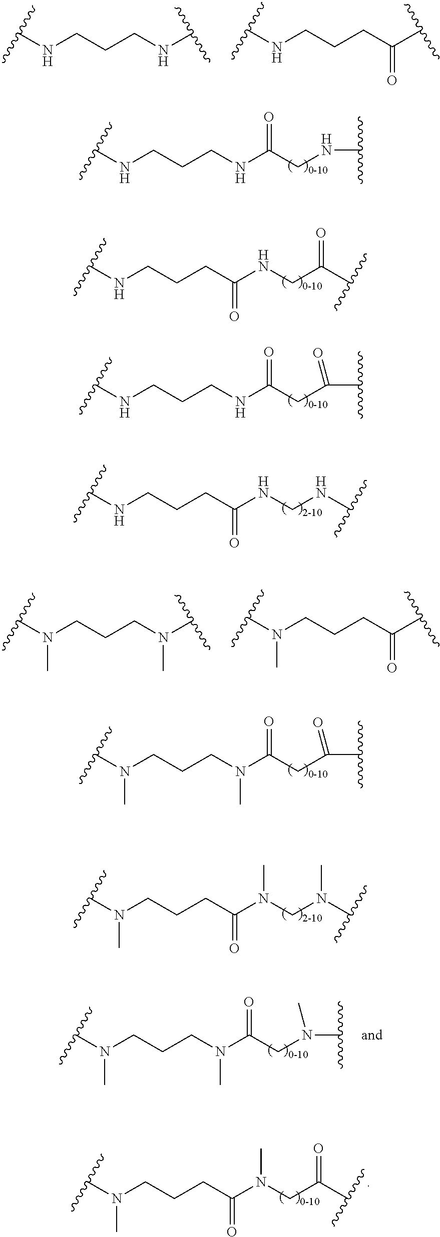

In certain embodiments, Linker-B is selected from:

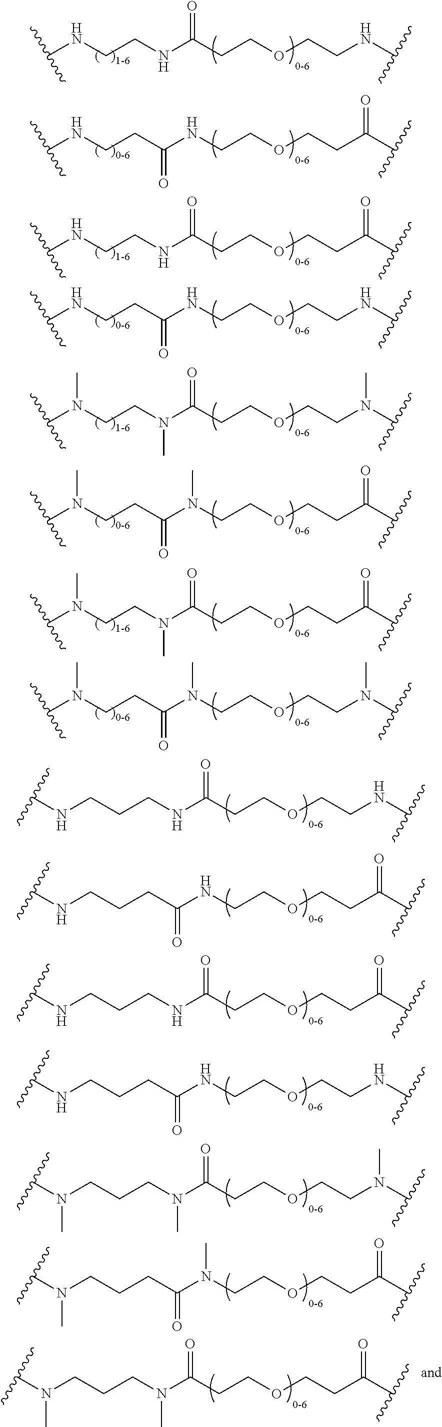

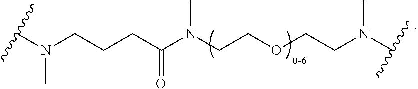

In certain embodiments, Linker-B is selected from:

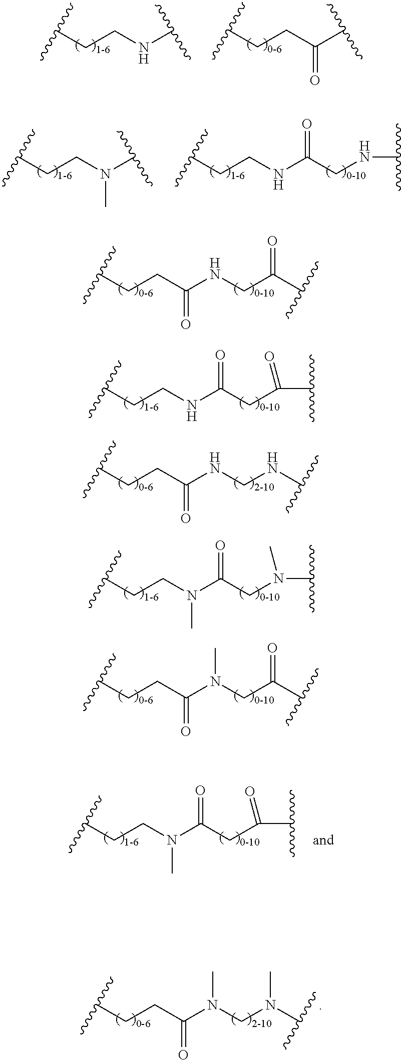

In certain embodiments, Linker-B is selected from:

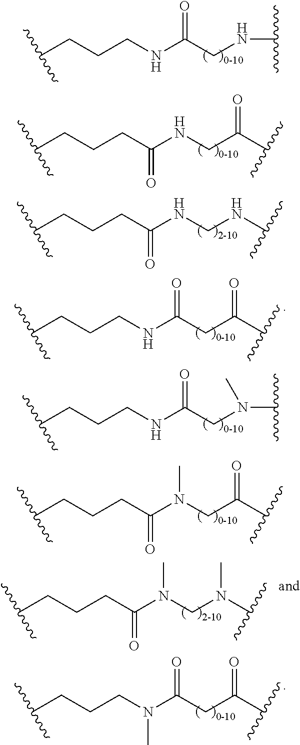

In certain embodiments, Linker-B is selected from:

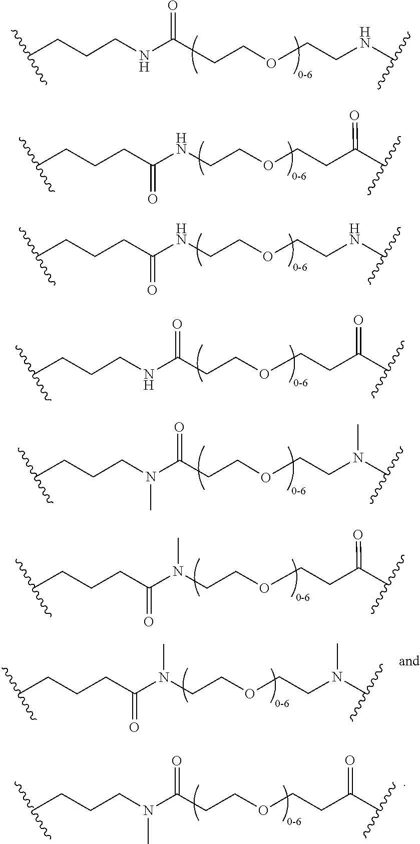

In certain embodiments, Linker-B is selected from:

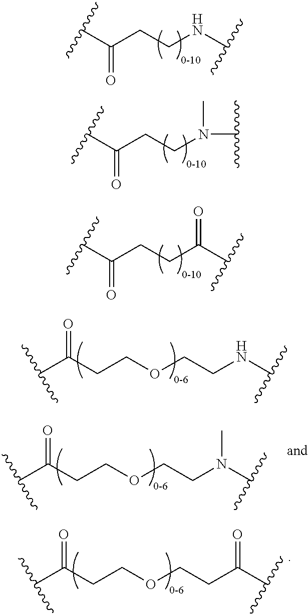

In certain embodiments, Linker-B is selected from:

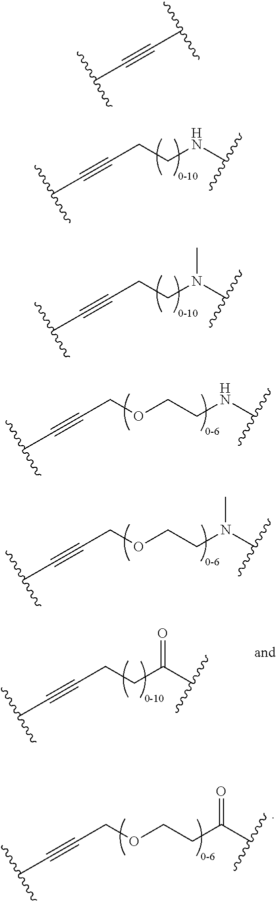

In certain embodiments, Linker-B is selected from:

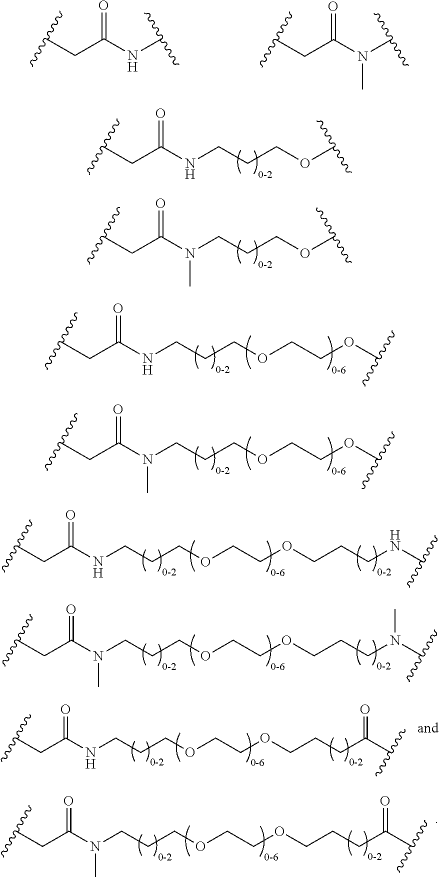

In certain embodiments, Linker-B is selected from:

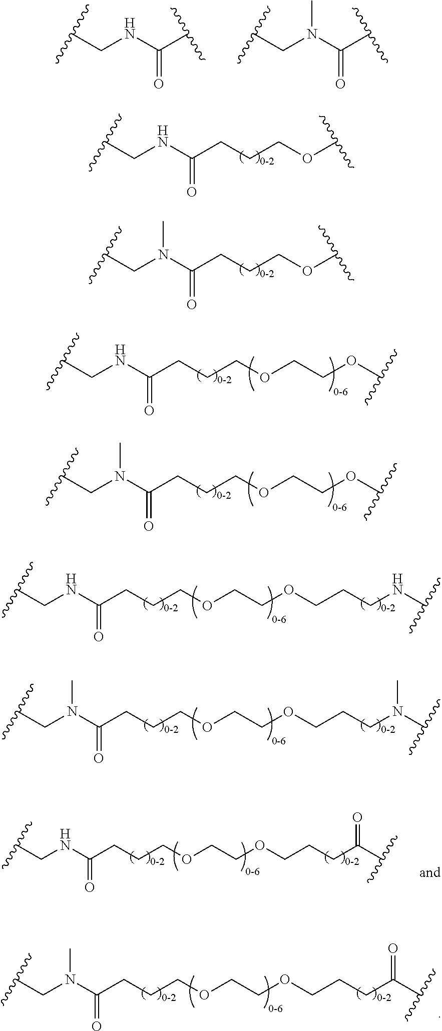

In certain embodiments, Linker-B is selected from:

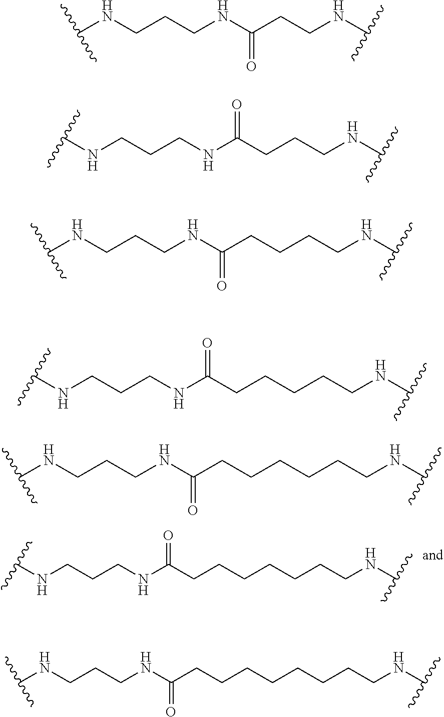

In certain embodiments, Linker-B is selected from:

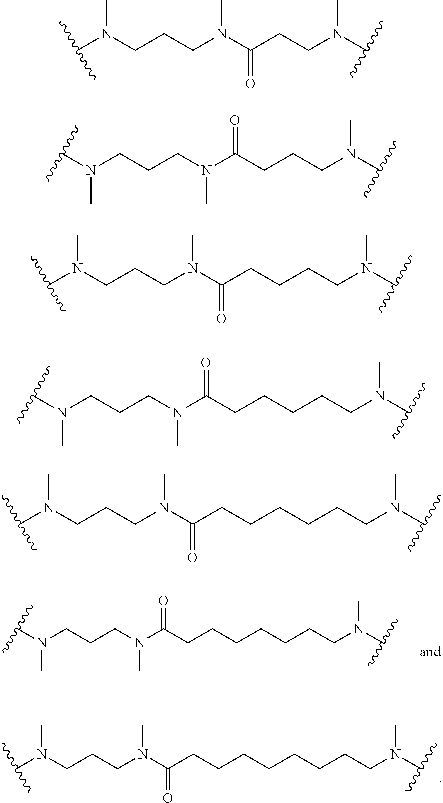

In certain embodiments, Linker-B is selected from:

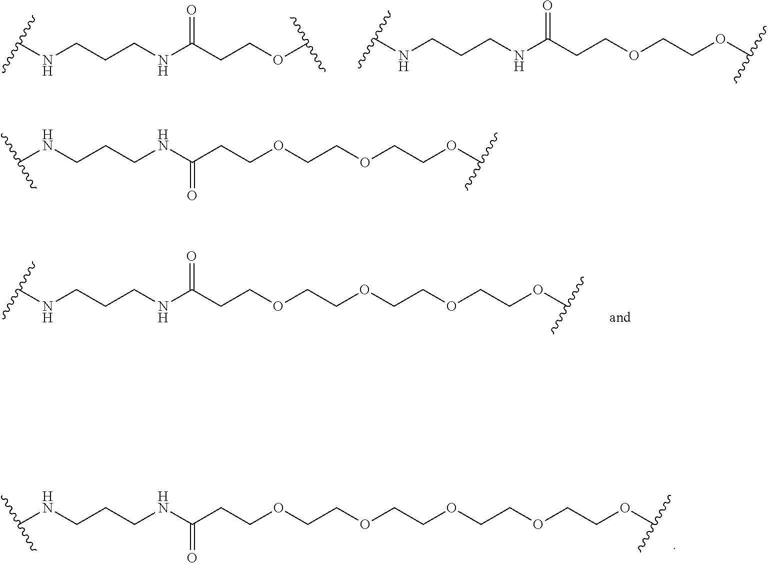 [SMILES: *NCCCNC(=O)CCO* ; *NCCCNC(=O)CCOCCO* ; *NCCCNC(=O)CCOCCOCCO* ; *NCCCNC(=O)CCOCCOCCOCCO* ; *NCCCNC(=O)CCOCCOCCOCCOCCO*]

In certain embodiments, Linker-B is selected from:

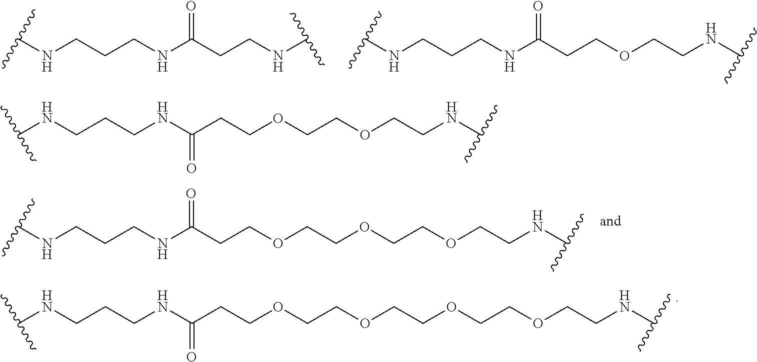

In certain embodiments, Linker-B is selected from:

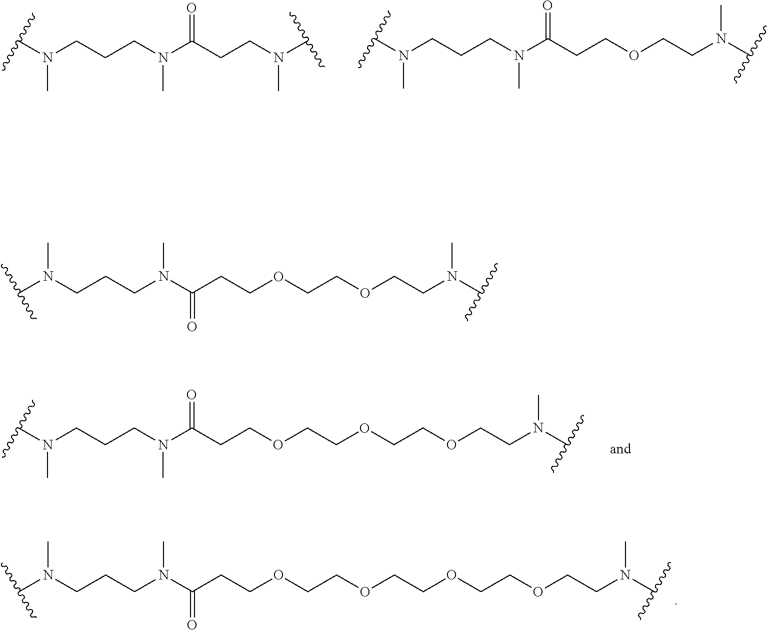 [SMILES: *N(C)CCCN(C)C(=O)CCN(*)C ; *N(C)CCCN(C)C(=O)CCOCCN(*)C ; *N(C)CCCN(C)C(=O)CCOCCOCCN(*)C ; *N(C)CCCN(C)C(=O)CCOCCOCCOCCN(*)C ; *N(C)CCCN(C)C(=O)CCOCCOCCOCCOCCN(*)C]

In certain embodiments, Linker-B is selected from:

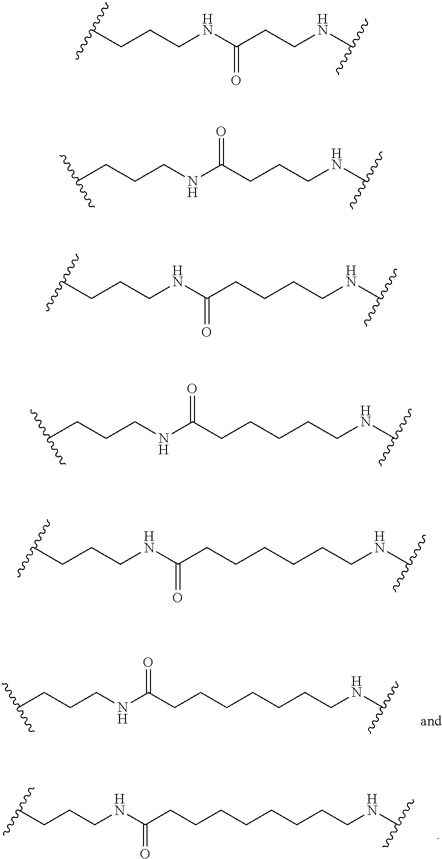

In certain embodiments, Linker-B is selected from:

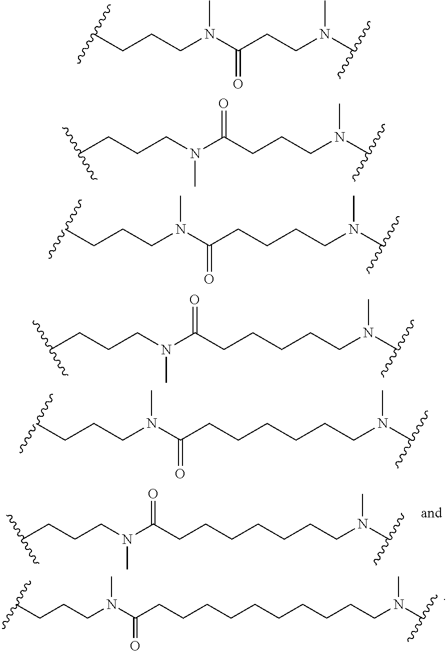

In certain embodiments, Linker-B is selected from:

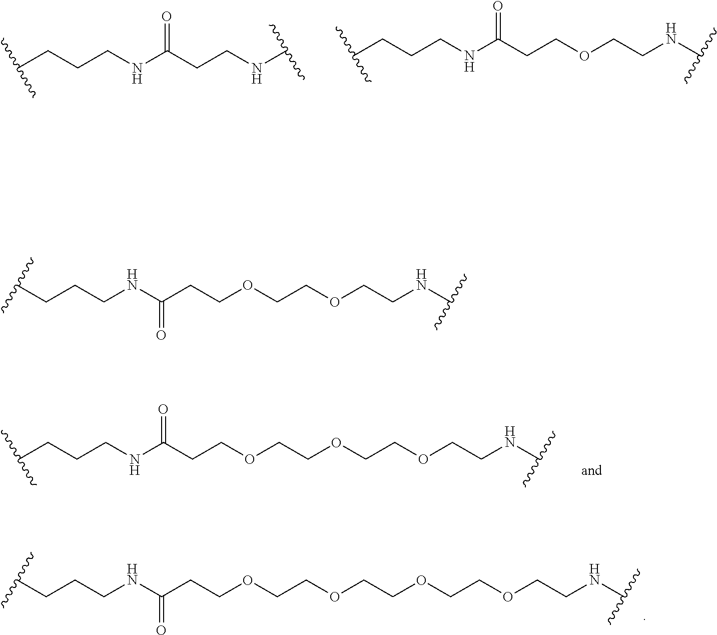 [SMILES: *CCCNC(=O)CCN* ; *CCCNC(=O)CCOCCN* ; *CCCNC(=O)CCOCCOCCN* ; *CCCNC(=O)CCOCCOCCOCCN* ; *CCCNC(=O)CCOCCOCCOCCOCCN*]

In certain embodiments, Linker-B is selected from:

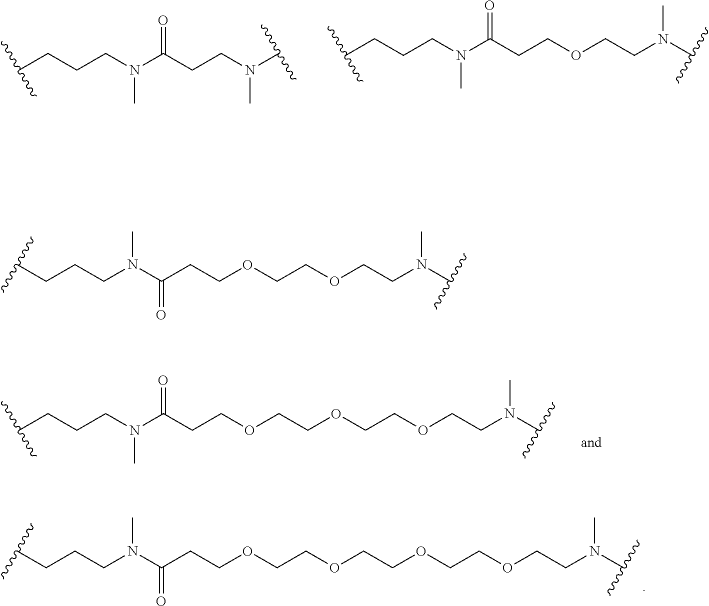 [SMILES: *CCCN(C)C(=O)CCN(*)C ; *CCCN(C)C(=O)CCOCCN(*)C ; *CCCN(C)C(=O)CCOCCOCCN(*)C ; *CCCN(C)C(=O)CCOCCOCCOCCN(*)C ; *CCCN(C)C(=O)CCOCCOCCOCCOCCN(*)C]

In certain embodiments, Linker-B is selected from:

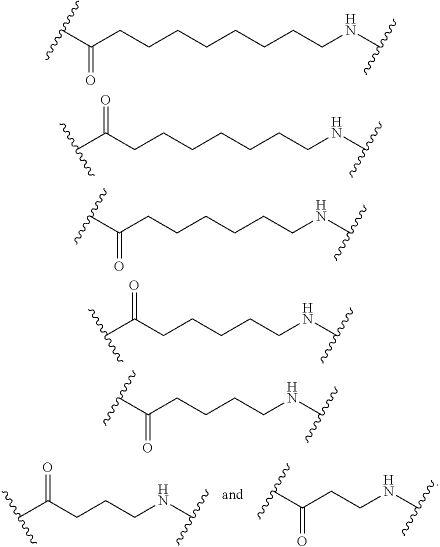

In certain embodiments, Linker-B is selected from:

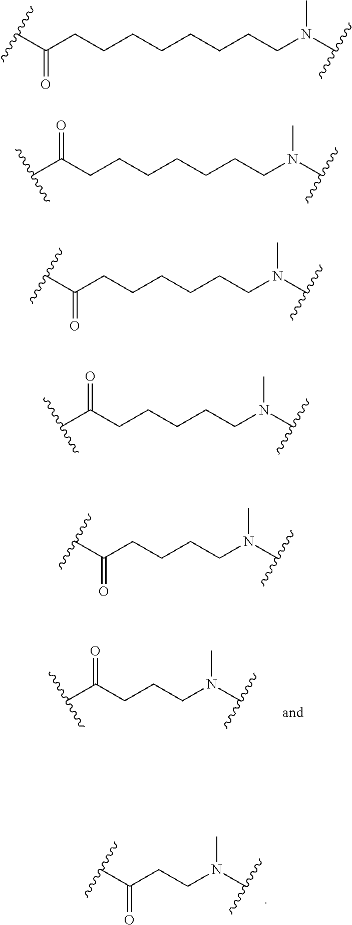

In certain embodiments, Linker-B is selected from:

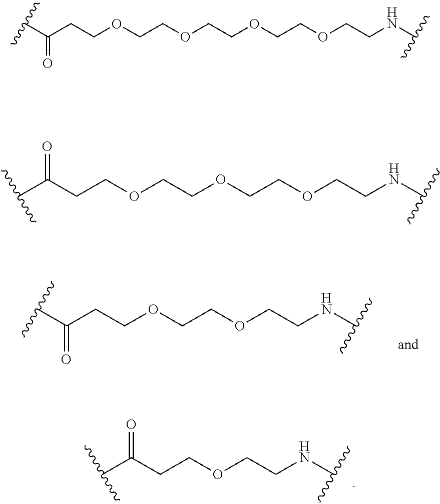 [SMILES: *NCCOCCOCCOCCOCCC(*)=O ; *NCCOCCOCCOCCC(*)=O ; *NCCOCCOCCC(*)=O ; *NCCOCCC(*)=O]

In certain embodiments, Linker-B is selected from:

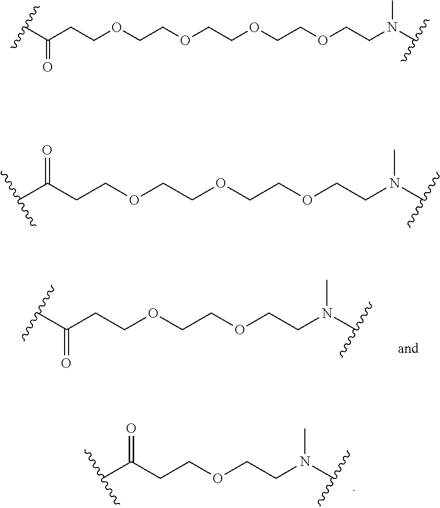 [SMILES: *C(=O)CCOCCOCCOCCOCCN(*)C ; *C(=O)CCOCCOCCOCCN(*)C ; *C(=O)CCOCCOCCN(*)C ; *C(=O)CCOCCN(*)C]

In certain embodiments, Linker-B is selected from:

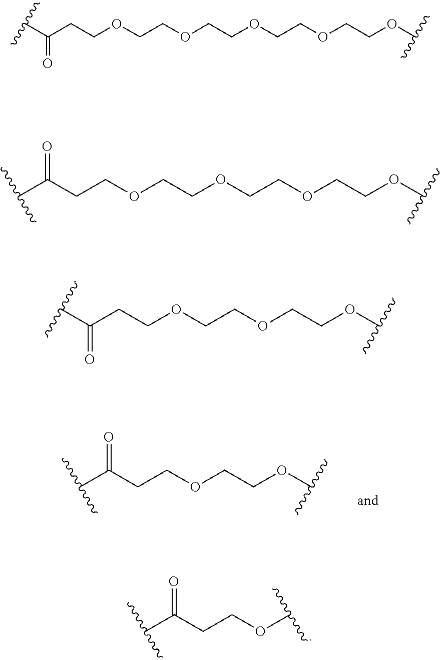 [SMILES: *OCCOCCOCCOCCOCCC(*)=O ; *OCCOCCOCCOCCC(*)=O ; *OCCOCCOCCC(*)=O ; *OCCOCCC(*)=O ; *OCCC(*)=O]

In certain embodiments, Linker-B is selected from:

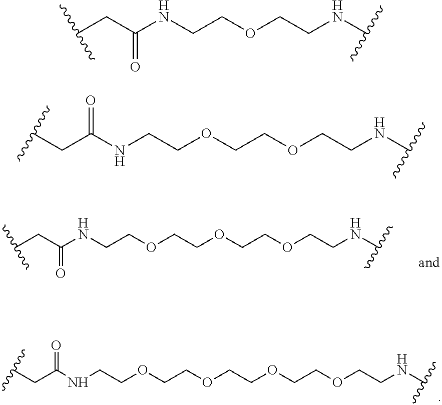 [SMILES: *CC(=O)NCCOCCN* ; *CC(=O)NCCOCCOCCN* ; *CC(=O)NCCOCCOCCOCCN* ; *CC(=O)NCCOCCOCCOCCOCCN*]

In certain embodiments, Linker-B is selected from:

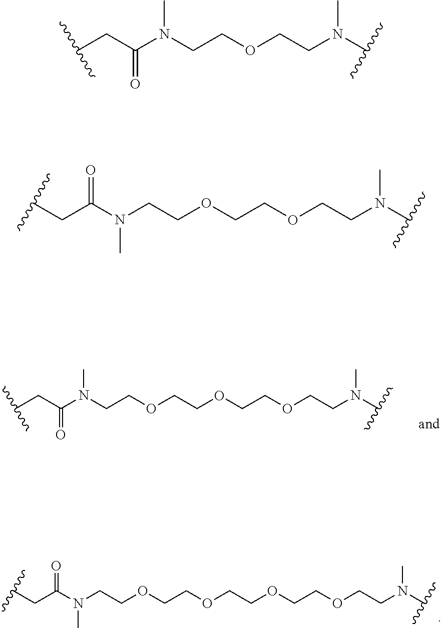 [SMILES: *CC(=O)N(C)CCOCCN(*)C ; *CC(=O)N(C)CCOCCOCCN(*)C ; *CC(=O)N(C)CCOCCOCCOCCN(*)C ; *CC(=O)N(C)CCOCCOCCOCCOCCN(*)C]

In certain embodiments, Linker-B is selected from:

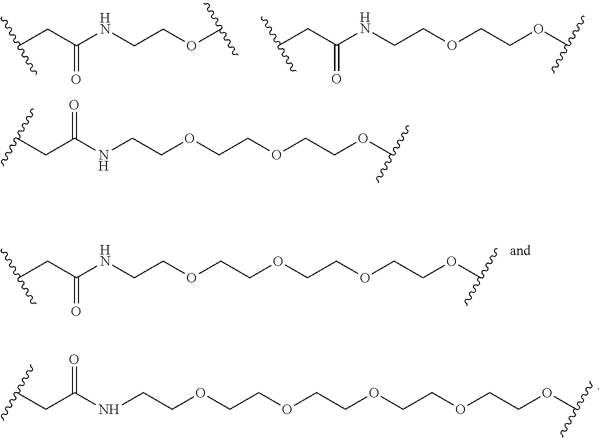

In certain embodiments, Linker-B is selected from:

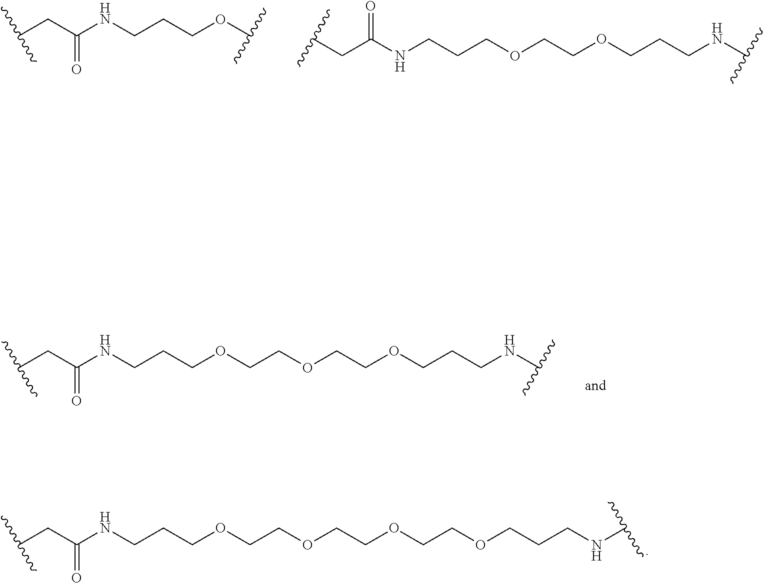 [SMILES: *CC(=O)NCCCO* ; *CC(=O)NCCCOCCOCCCN* ; *CC(=O)NCCCOCCOCCOCCCN* ; *CC(=O)NCCCOCCOCCOCCOCCCN*]

In certain embodiments, Linker-B is selected from:

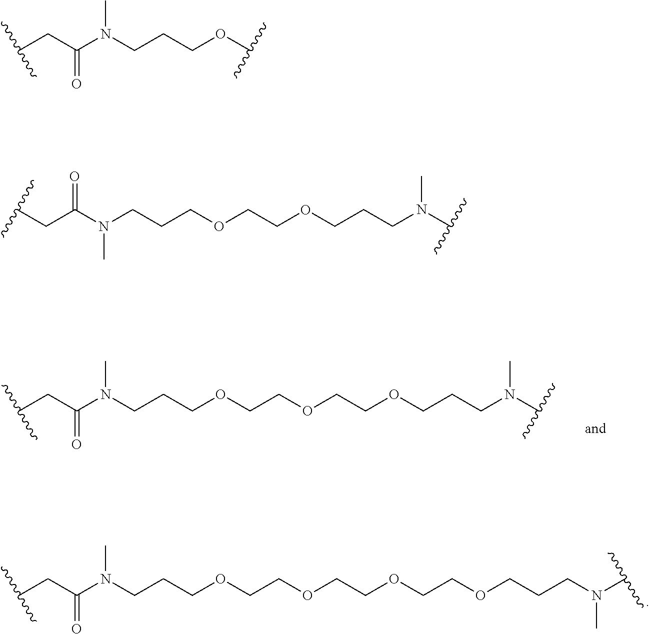 [SMILES: *CC(=O)N(C)CCCO* ; *CC(=O)N(C)CCCOCCOCCCN(*)C ; *CC(=O)N(C)CCCOCCOCCOCCCN(*)C ; *CC(=O)N(C)CCCOCCOCCOCCOCCCN(*)C]

In certain embodiments, Linker-B is selected from:

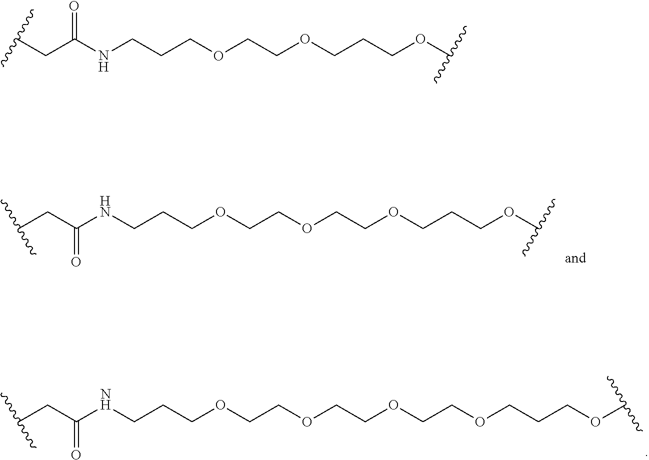 [SMILES: *CC(=O)NCCCOCCOCCCO* ; *CC(=O)NCCCOCCOCCOCCCO* ; *CC(=O)NCCCOCCOCCOCCOCCCO*]

In certain embodiments, Linker-B is selected from:

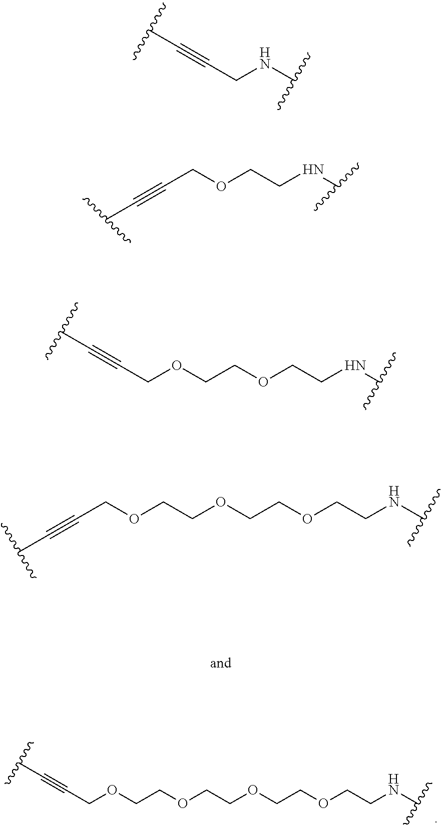 [SMILES: *C#CCN* ; *C#CCOCCN* ; *C#CCOCCOCCN* ; *C#CCOCCOCCOCCN* ; *C#CCOCCOCCOCCOCCN*]

In certain embodiments, Linker-B is selected from:

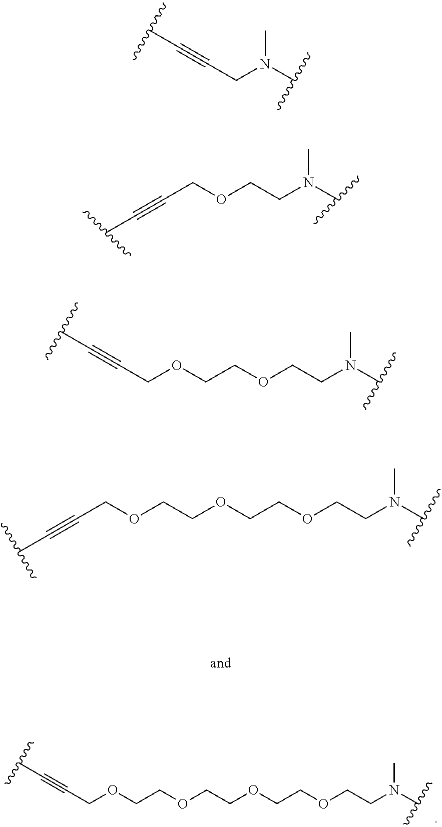

In certain embodiments, Linker-B is selected from:

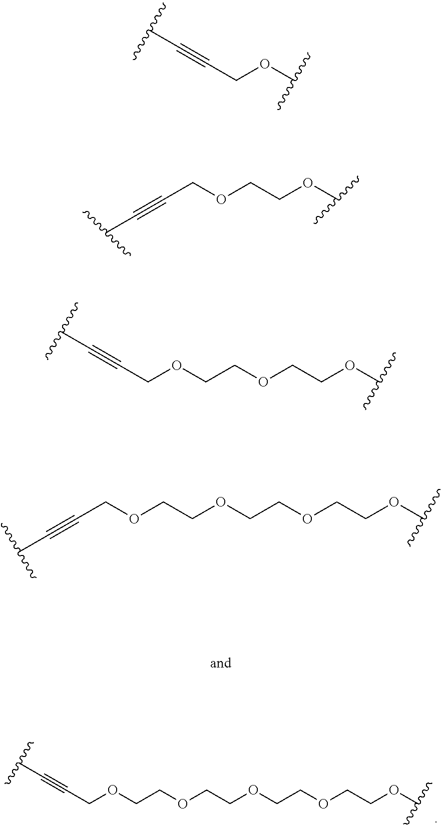 [SMILES: *C#CCO* ; *C#CCOCCO* ; *C#CCOCCOCCO* ; *C#CCOCCOCCOCCO* ; *C#CCOCCOCCOCCOCCO*]

In certain embodiments Linker-A and/or Linker-B is selected from:

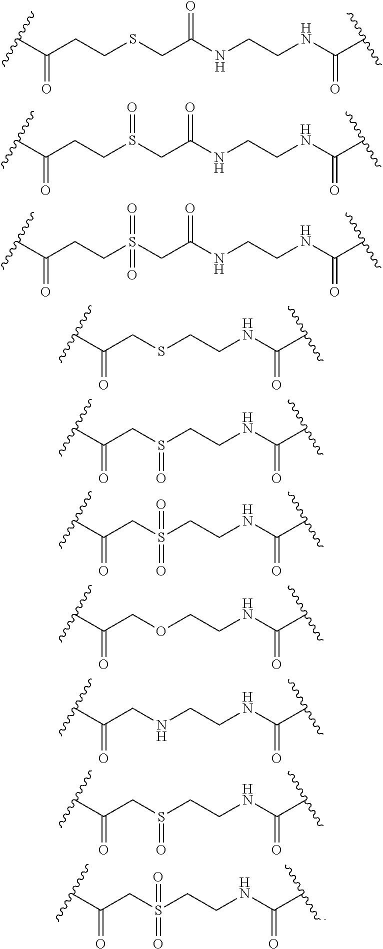

In certain embodiments Linker-A and/or Linker-B is selected from:

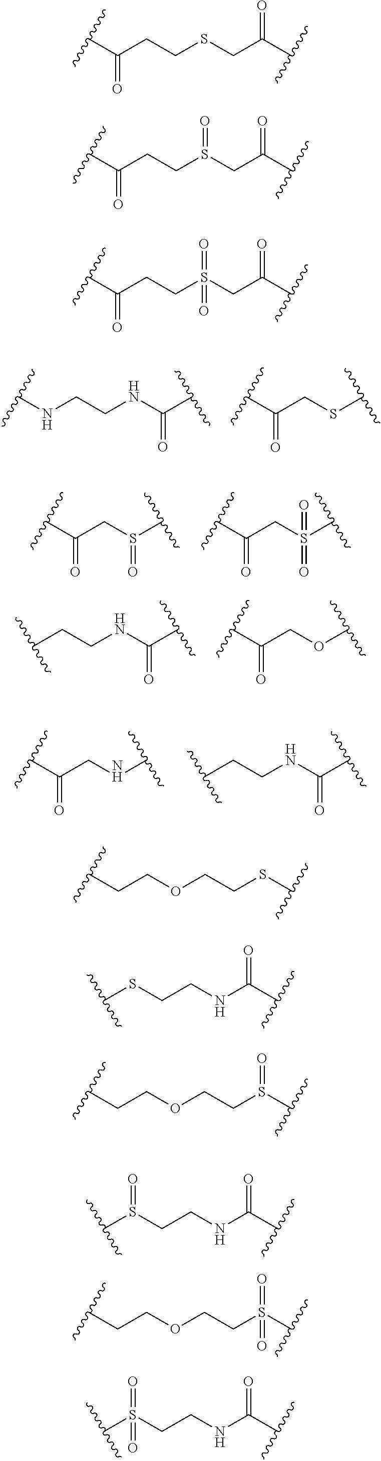

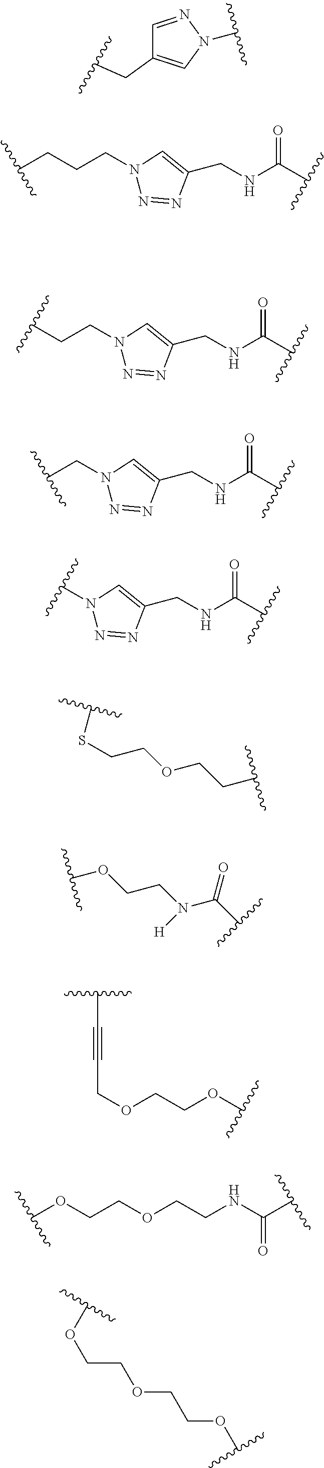

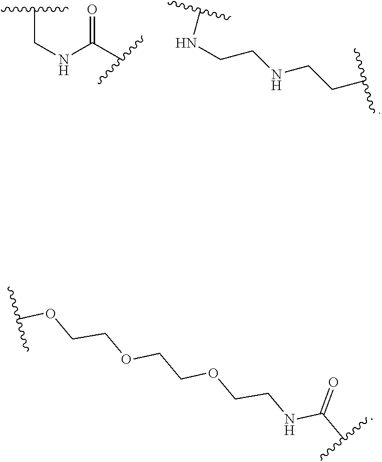 [SMILES: *CNC(*)=O ; *CCNCCN* ; *OCCOCCOCCNC(*)=O]

### USP7 Targeting Ligands

In certain embodiments, the crystal structure of USP7 is searchable by 4 WPH, 4WPI, 1YZE, 4M5X, and 4PYZ (Pfoh et al., “Crystal Structure of USP7 Ubiquitin-like Domains with an ICP0 Peptide Reveals a Novel Mechanism Used by Viral and Cellular Proteins to Target USP7”, PLoS Pathog., 2015, 11: e1004950-e1004950; Saridakis et al., “Structure of the p53 binding domain of HAUSP/USP7 bound to Epstein-Barr nuclear antigen 1 implications for EBV-mediated immortalization”, Mol Cell., 2005, 18: 25-36; Molland et al., “A 2.2 angstrom resolution structure of the USP7 catalytic domain in a new space group elaborates upon structural rearrangements resulting from ubiquitin binding”, Acta Crystallogr Sect F Struct Biol Cryst Commun., 2014, 70: 283-287; Ong et al., “Crystal structure of the first two Ubl domains of Deubiquitylase USP7”, to be published).

Non-limiting examples of ligands that bind USP7 include those described in CN112812111A. In certain embodiments the USP7 Targeting Ligand used in Formula I or Formula II is a compound described in CN112812111A.

In certain embodiments the compound of the present invention is of Formula:

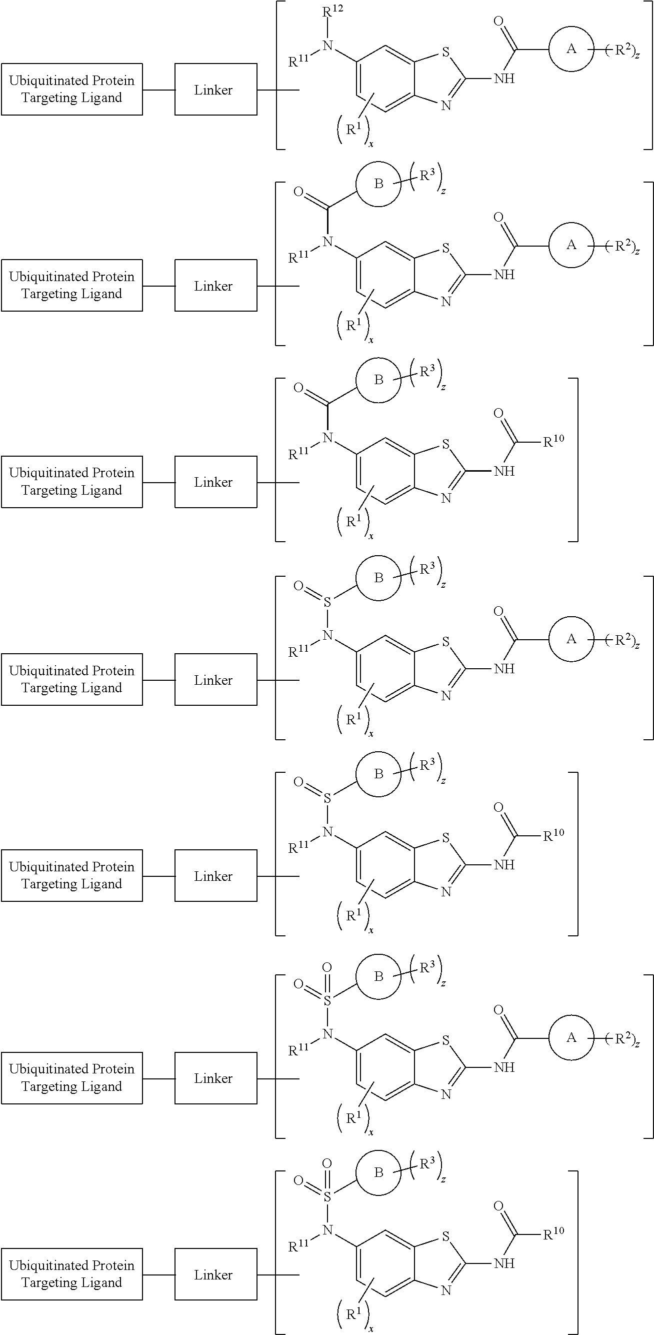

or a pharmaceutically acceptable salt thereof.

Non-limiting examples of ligands that bind USP7 include those described in WO2020086595A1. In certain embodiments the USP7 Targeting Ligand used in Formula I or Formula II is a compound described in WO2020086595A1.

In certain embodiments the compound of the present invention is of Formula:

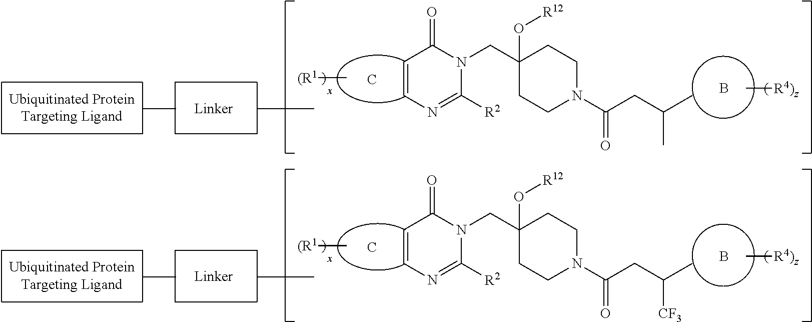

or a pharmaceutically acceptable salt thereof.

In certain embodiments the compound of the present invention is of Formula:

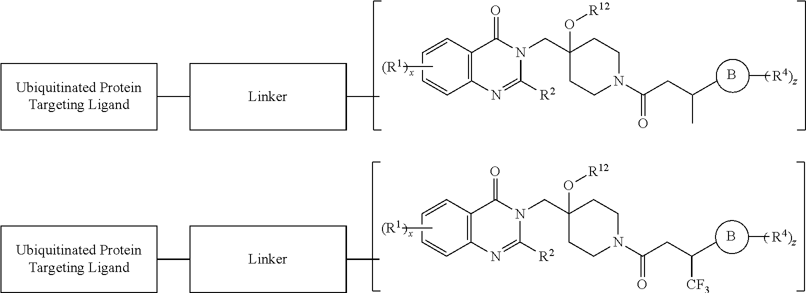

or a pharmaceutically acceptable salt thereof.

In certain embodiments the compound of the present invention is of Formula:

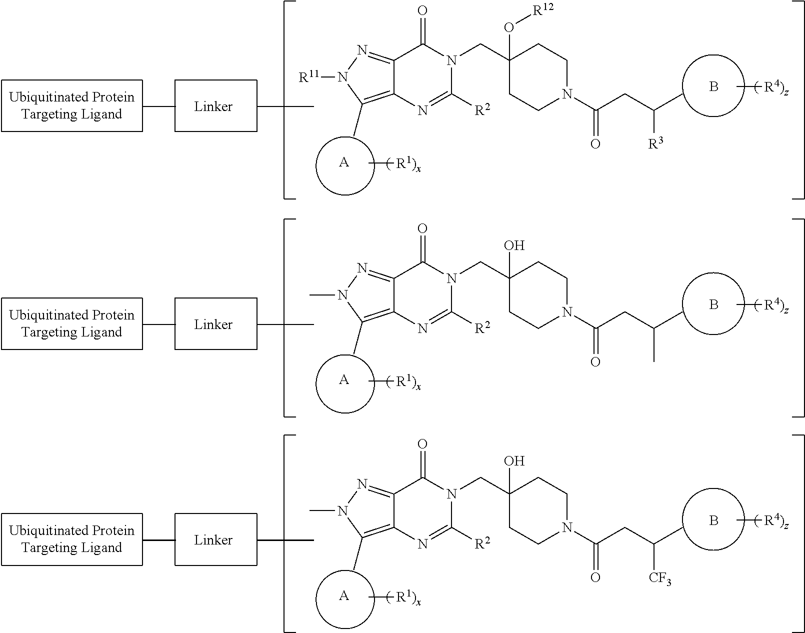

or a pharmaceutically acceptable salt thereof.

In certain embodiments the compound of the present invention is of Formula:

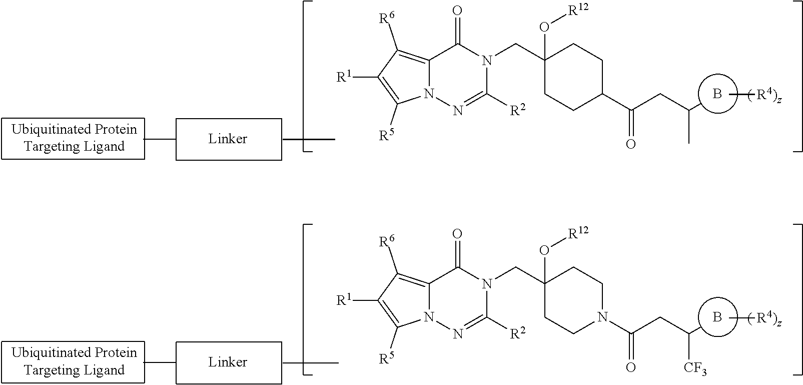

or a pharmaceutically acceptable salt thereof.

In certain embodiments the compound of the present invention is of Formula:

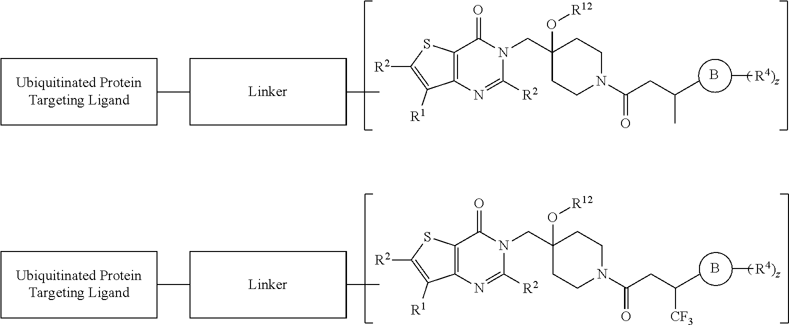

or a pharmaceutically acceptable salt thereof.

In certain embodiments the compound of the present invention is of Formula:

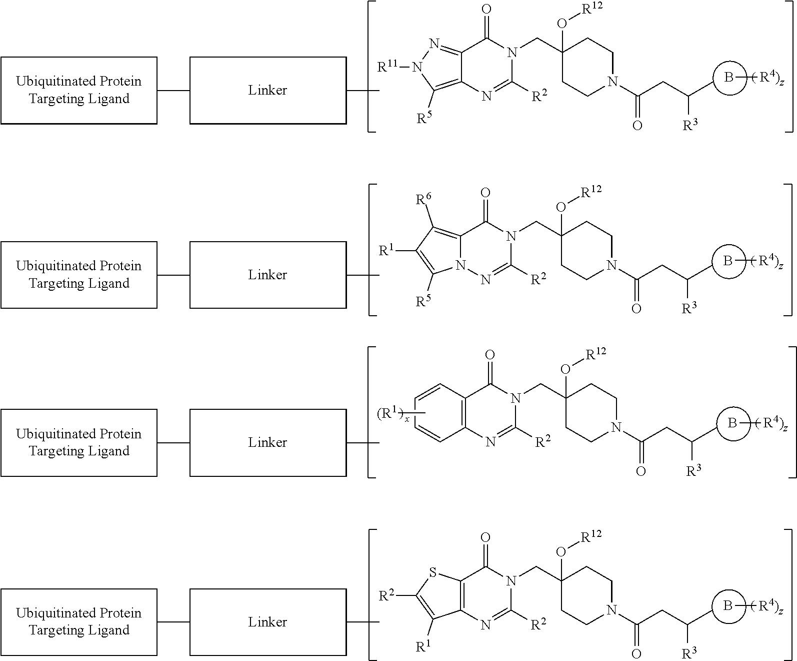

or a pharmaceutically acceptable salt thereof.

In certain embodiments the compound of the present invention is of Formula:

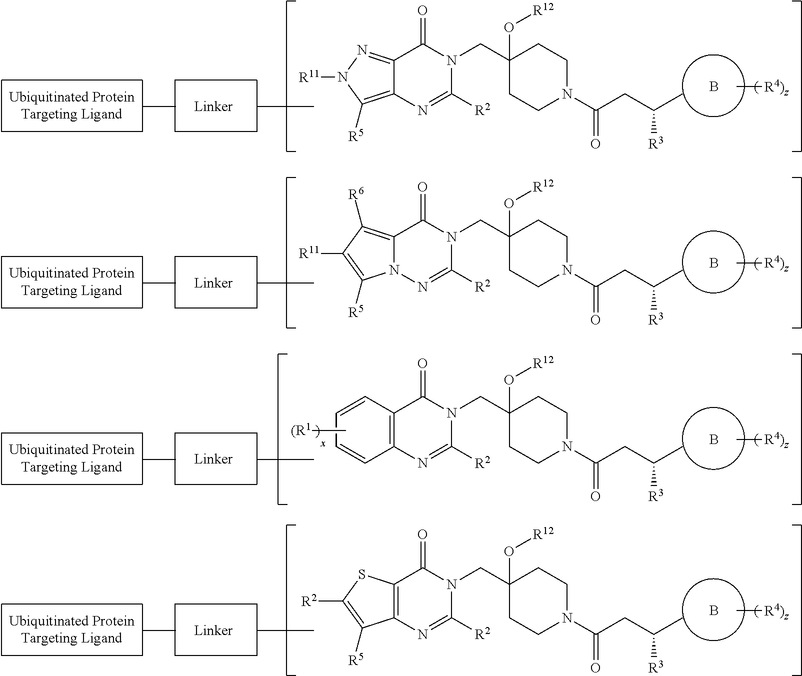

or a pharmaceutically acceptable salt thereof.

In certain embodiments the USP7 Targeting Ligand is selected from:

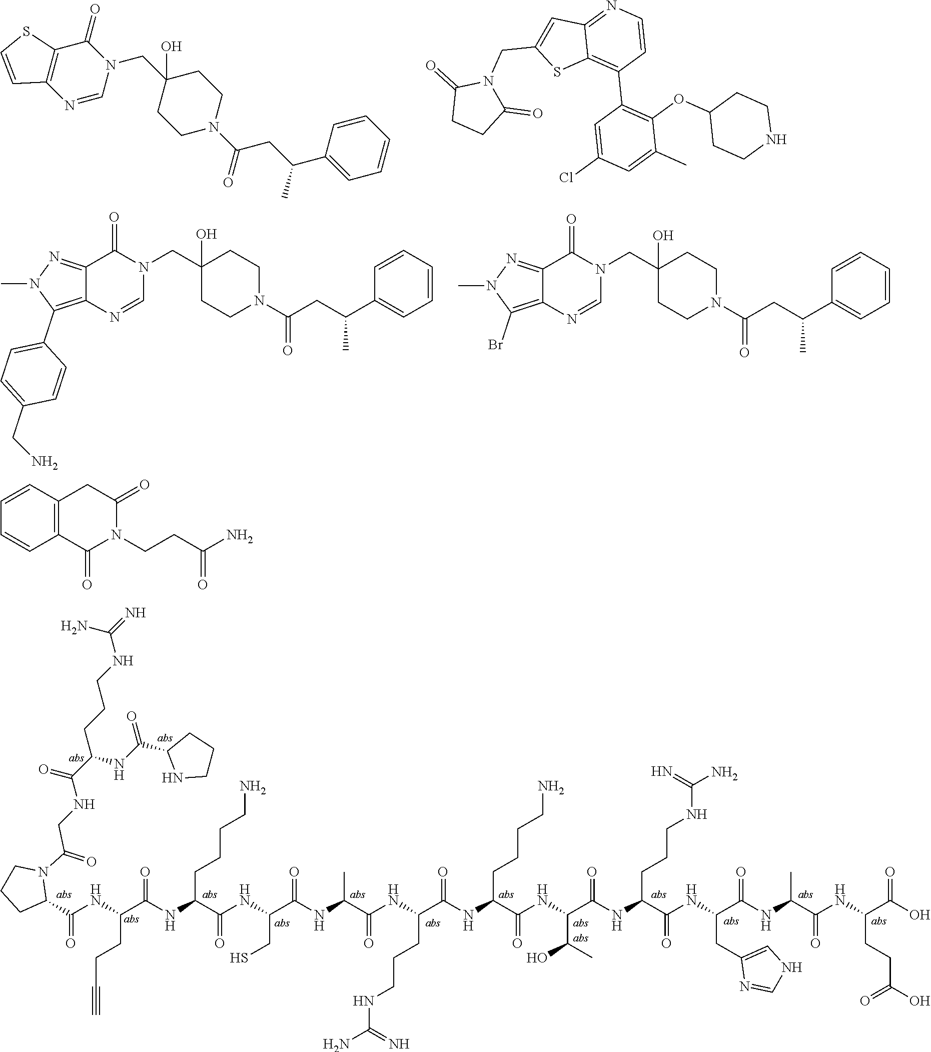

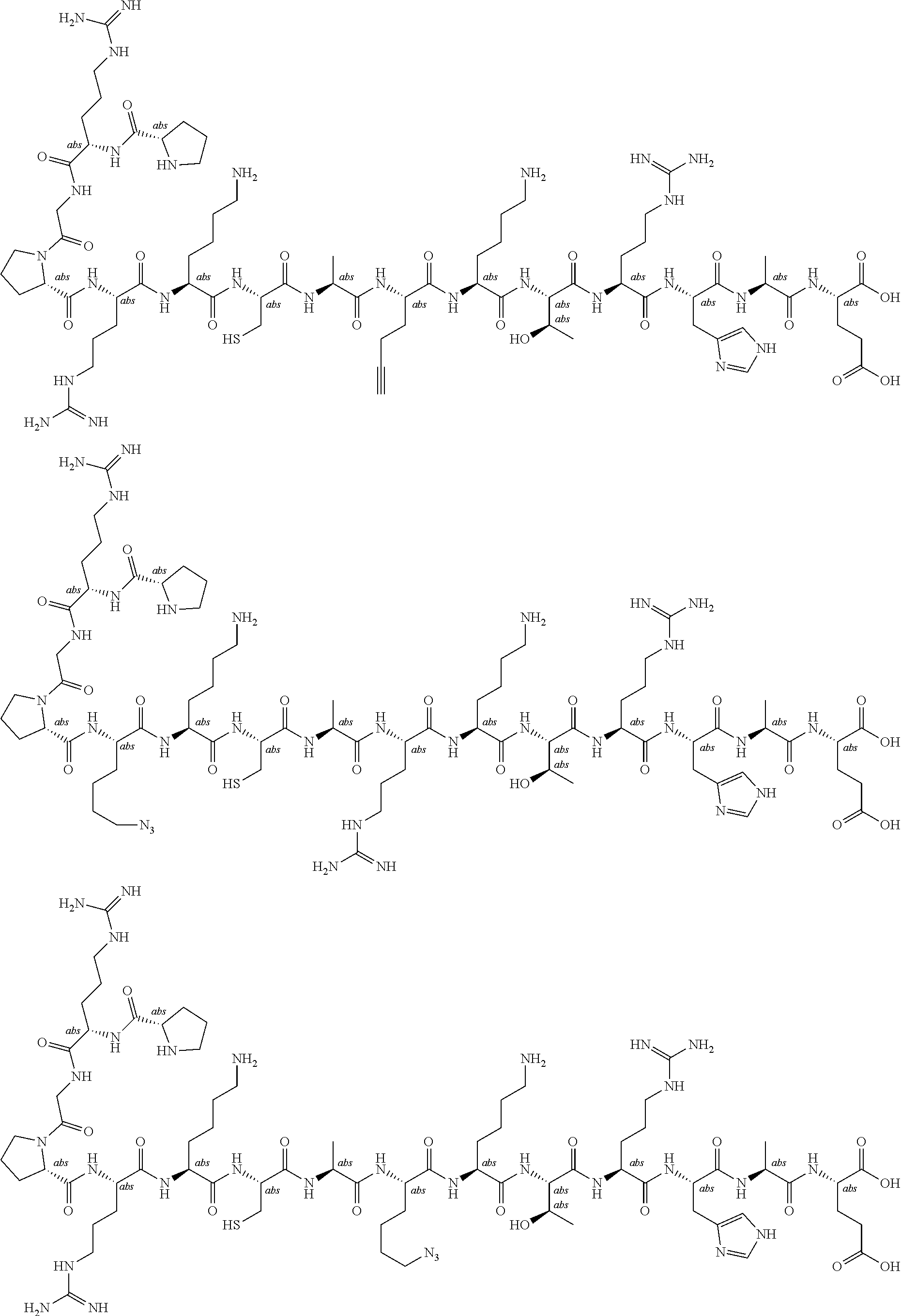

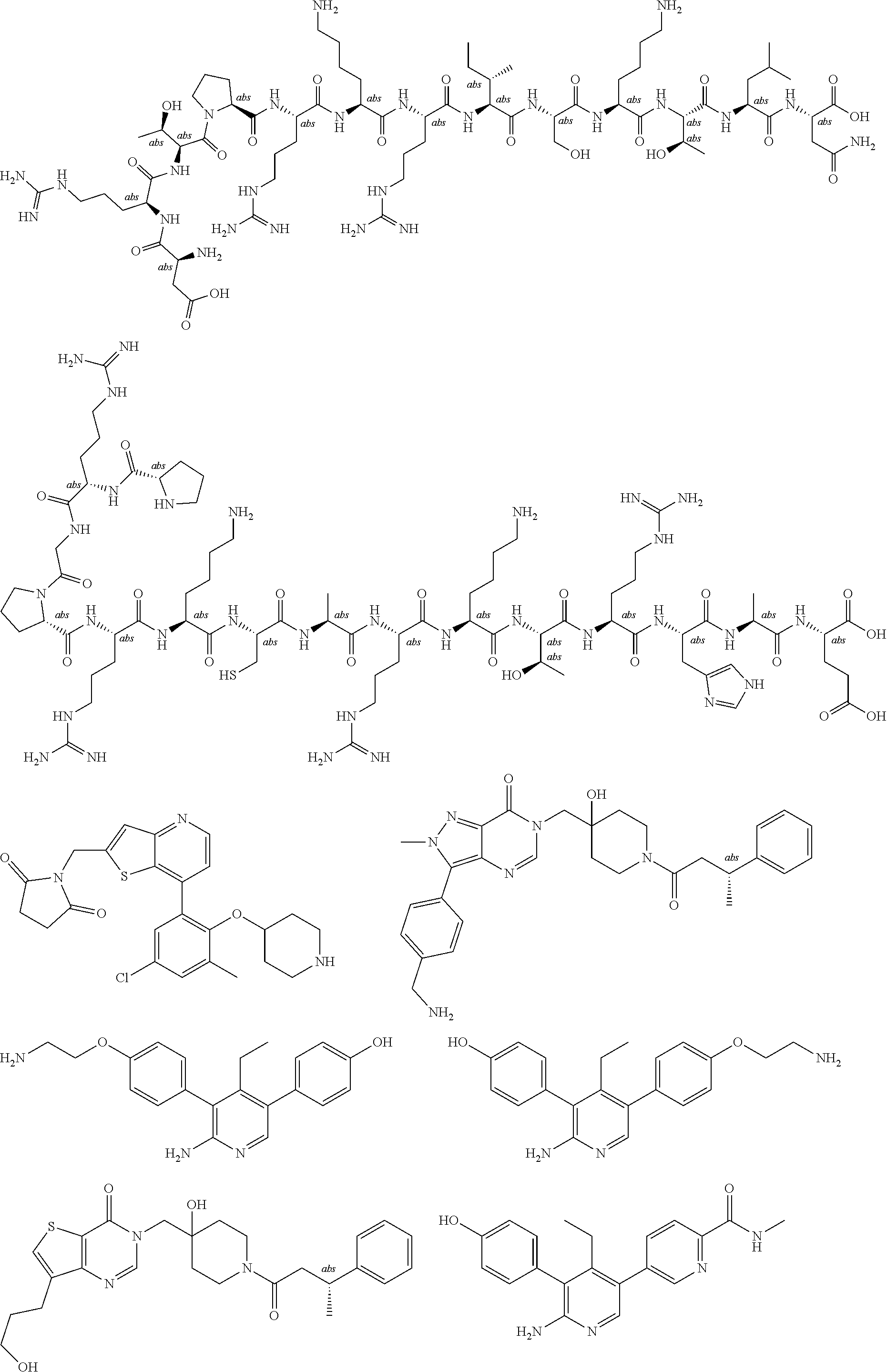

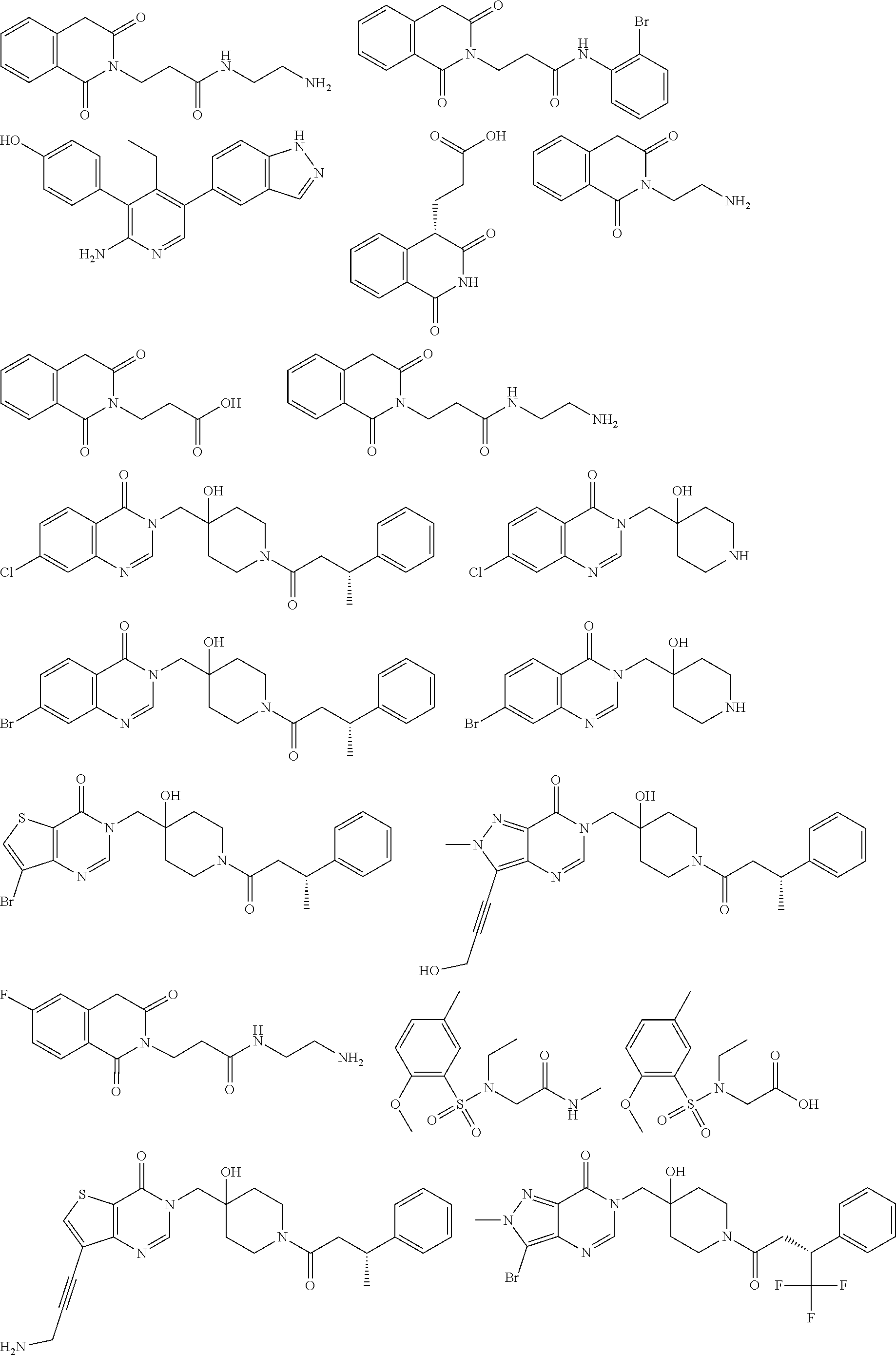

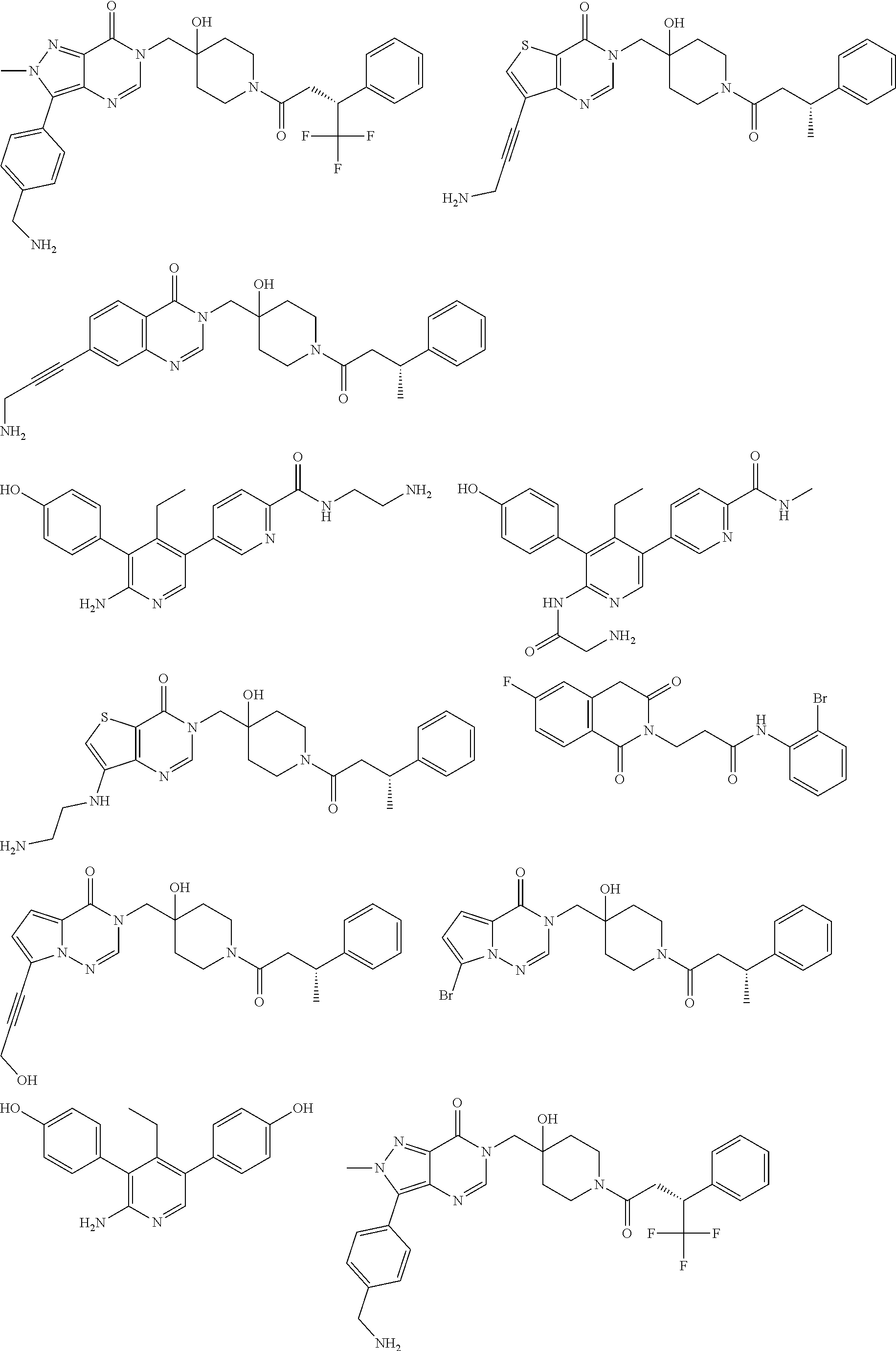

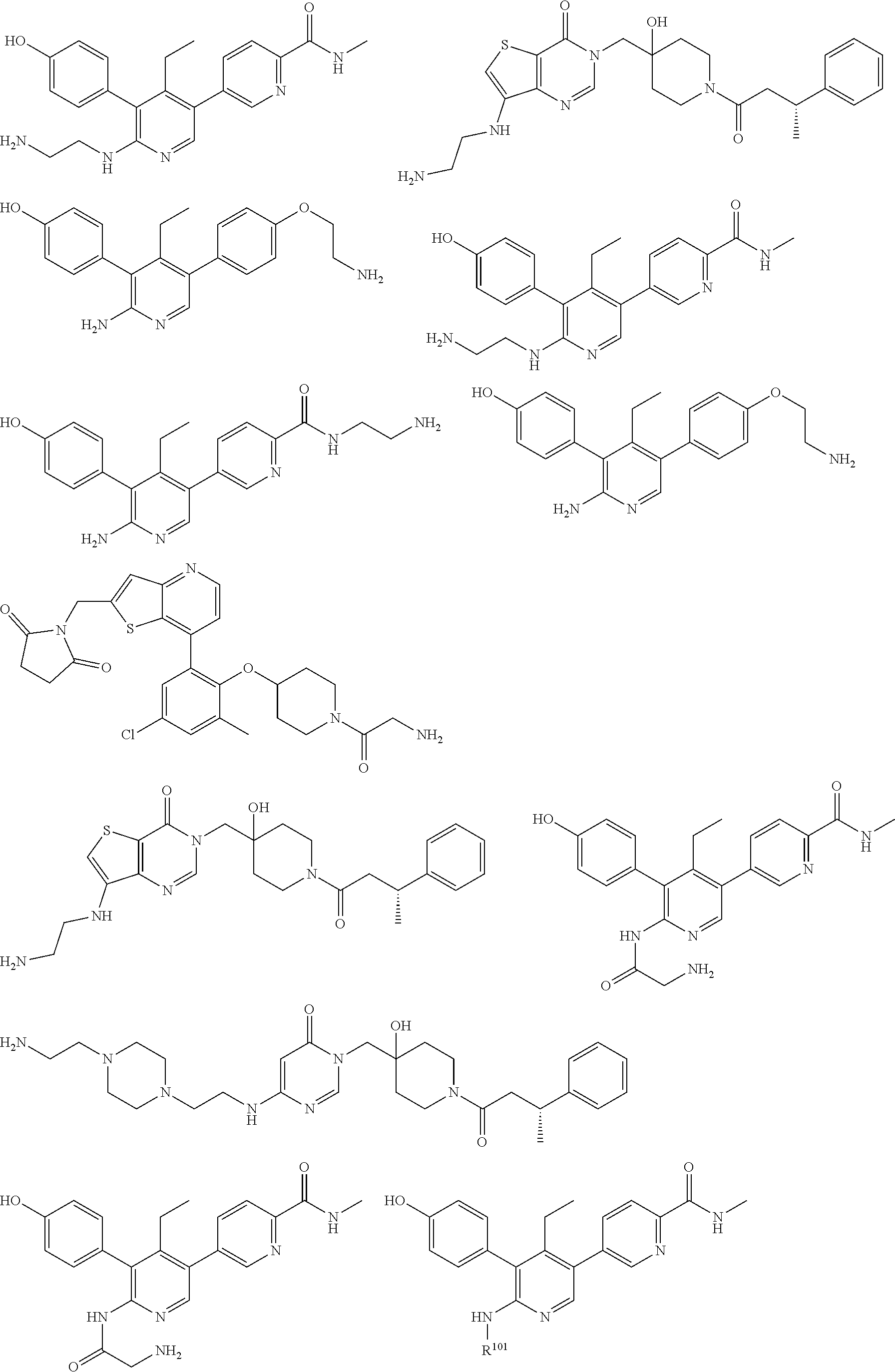

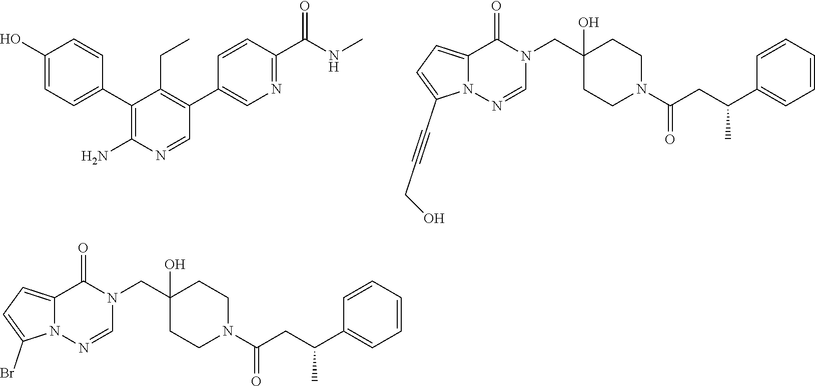 [SMILES: CCc1c(-c2ccc(C(=O)NC)nc2)cnc(N)c1-c1ccc(O)cc1 ; C[C@H](CC(=O)N1CCC(O)(Cn2cnn3c(C#CCO)ccc3c2=O)CC1)c1ccccc1 ; C[C@H](CC(=O)N1CCC(O)(Cn2cnn3c(Br)ccc3c2=O)CC1)c1ccccc1]

or a pharmaceutically acceptable salt thereof, wherein each of the above USP7 Targeting Ligands is substituted by 1-Linker-Ubiquitinated Protein Target Ligand and 0, 1, 2, or 3, R¹⁰¹ substituents; and
R¹⁰¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹.

### USP7 Targeting Ligand-Linker and USP7 Targeting Ligand-LinkerA

In certain embodiments the USP7 Targeting Ligand-Linker or USP7 Targeting Ligand-LinkerA group is selected from:

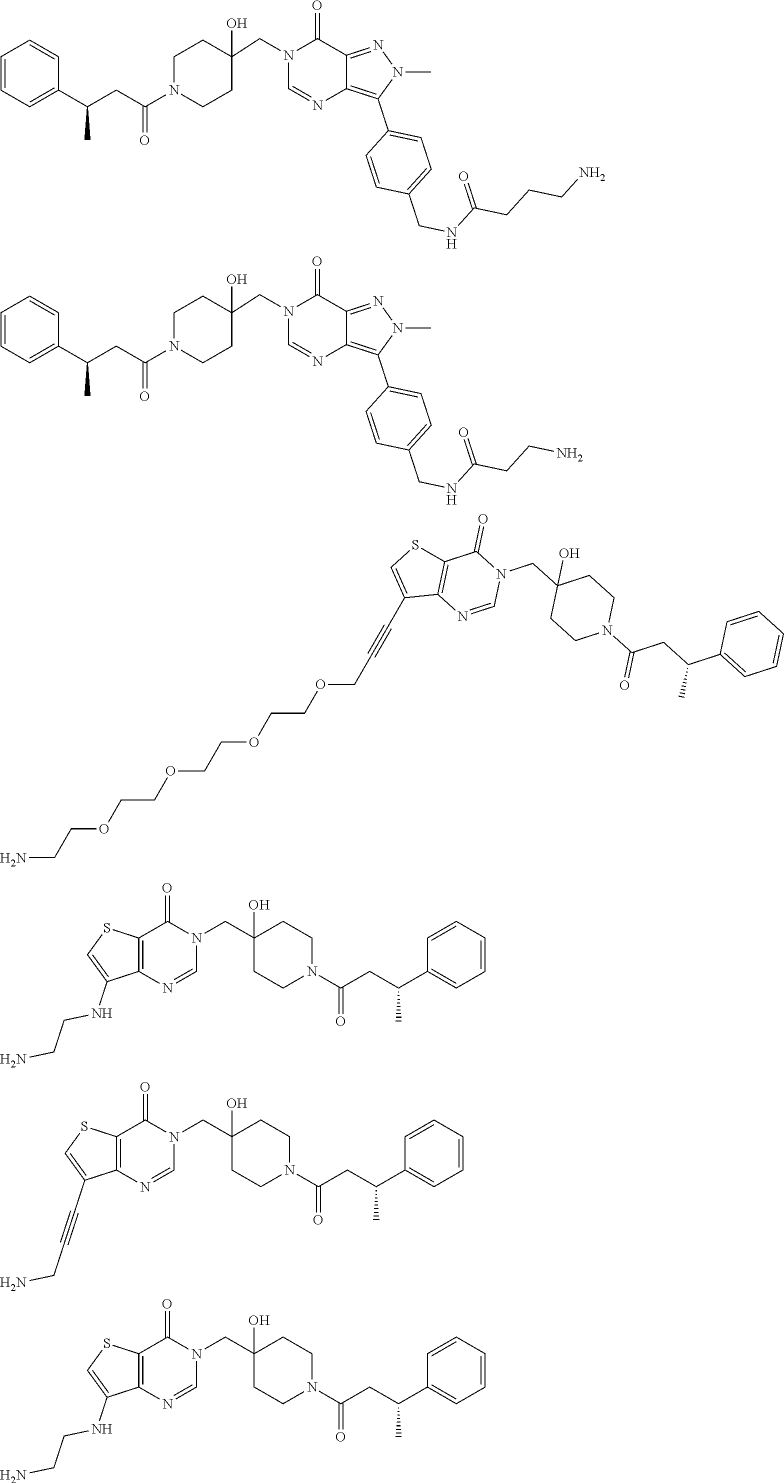

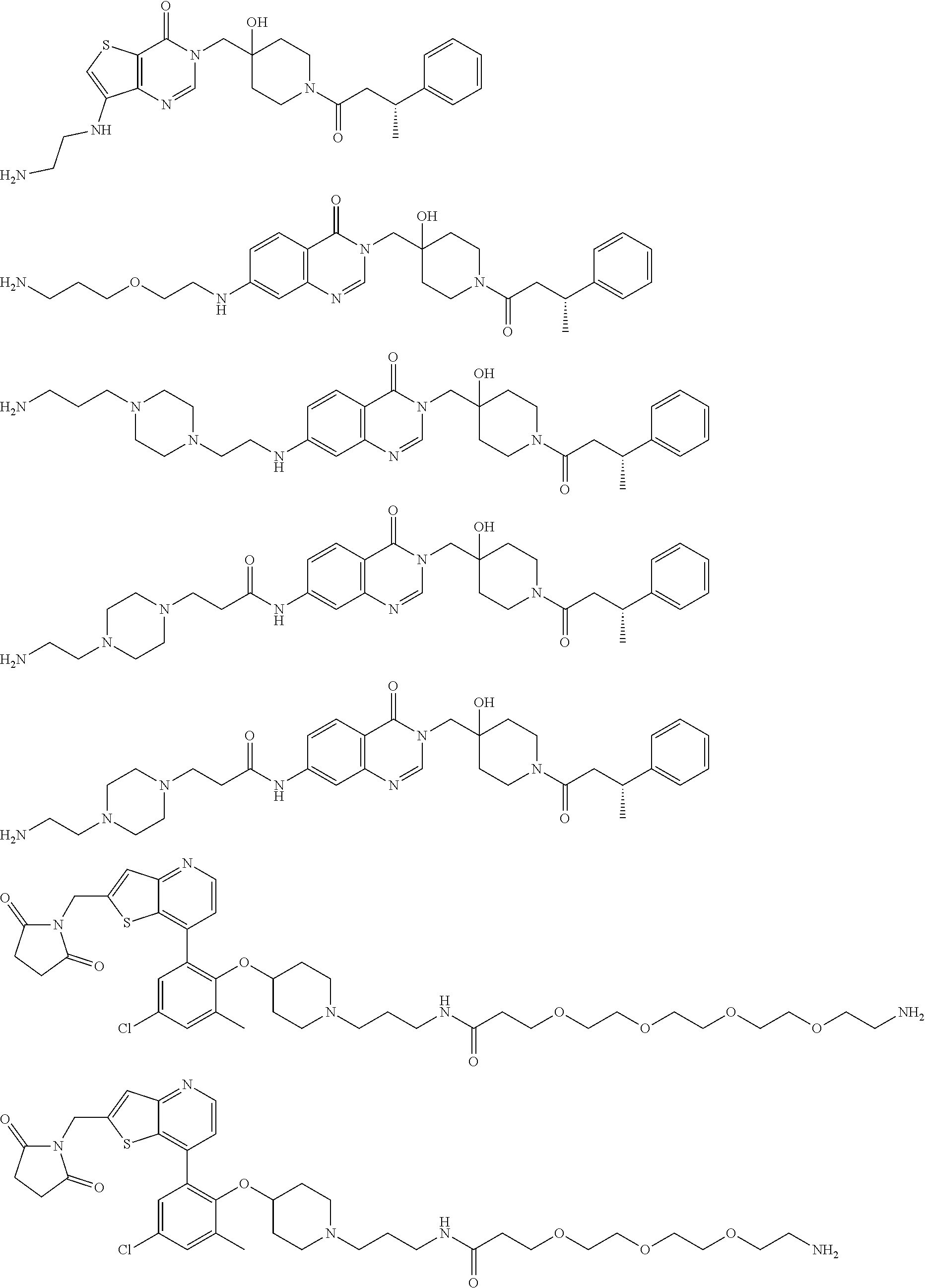

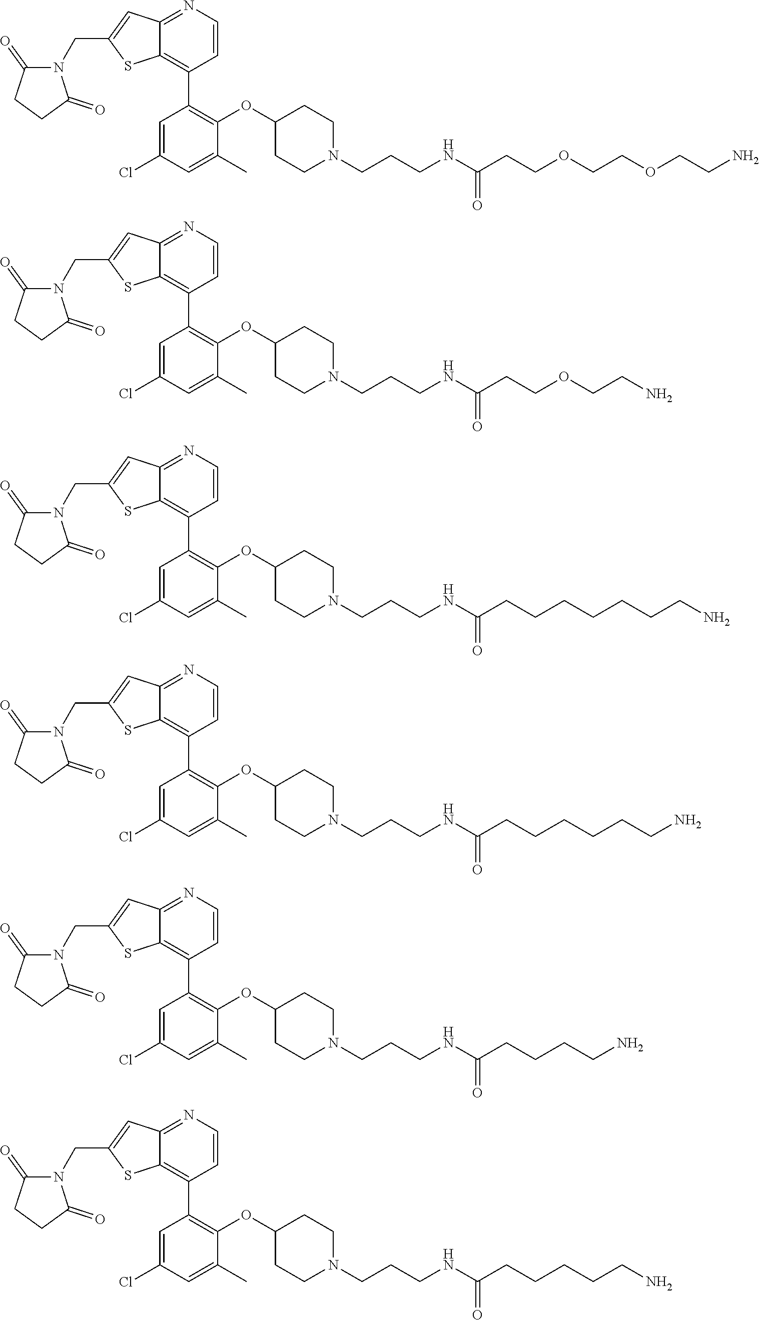 [SMILES: Cc1cc(Cl)cc(-c2ccnc3cc(CN4C(=O)CCC4=O)sc23)c1OC1CCN(CCCNC(=O)CCOCCOCCN)CC1 ; Cc1cc(Cl)cc(-c2ccnc3cc(CN4C(=O)CCC4=O)sc23)c1OC1CCN(CCCNC(=O)CCOCCN)CC1 ; Cc1cc(Cl)cc(-c2ccnc3cc(CN4C(=O)CCC4=O)sc23)c1OC1CCN(CCCNC(=O)CCCCCCCN)CC1 ; Cc1cc(Cl)cc(-c2ccnc3cc(CN4C(=O)CCC4=O)sc23)c1OC1CCN(CCCNC(=O)CCCCCCN)CC1 ; Cc1cc(Cl)cc(-c2ccnc3cc(CN4C(=O)CCC4=O)sc23)c1OC1CCN(CCCNC(=O)CCCCN)CC1 ; Cc1cc(Cl)cc(-c2ccnc3cc(CN4C(=O)CCC4=O)sc23)c1OC1CCN(CCCNC(=O)CCCCCN)CC1]

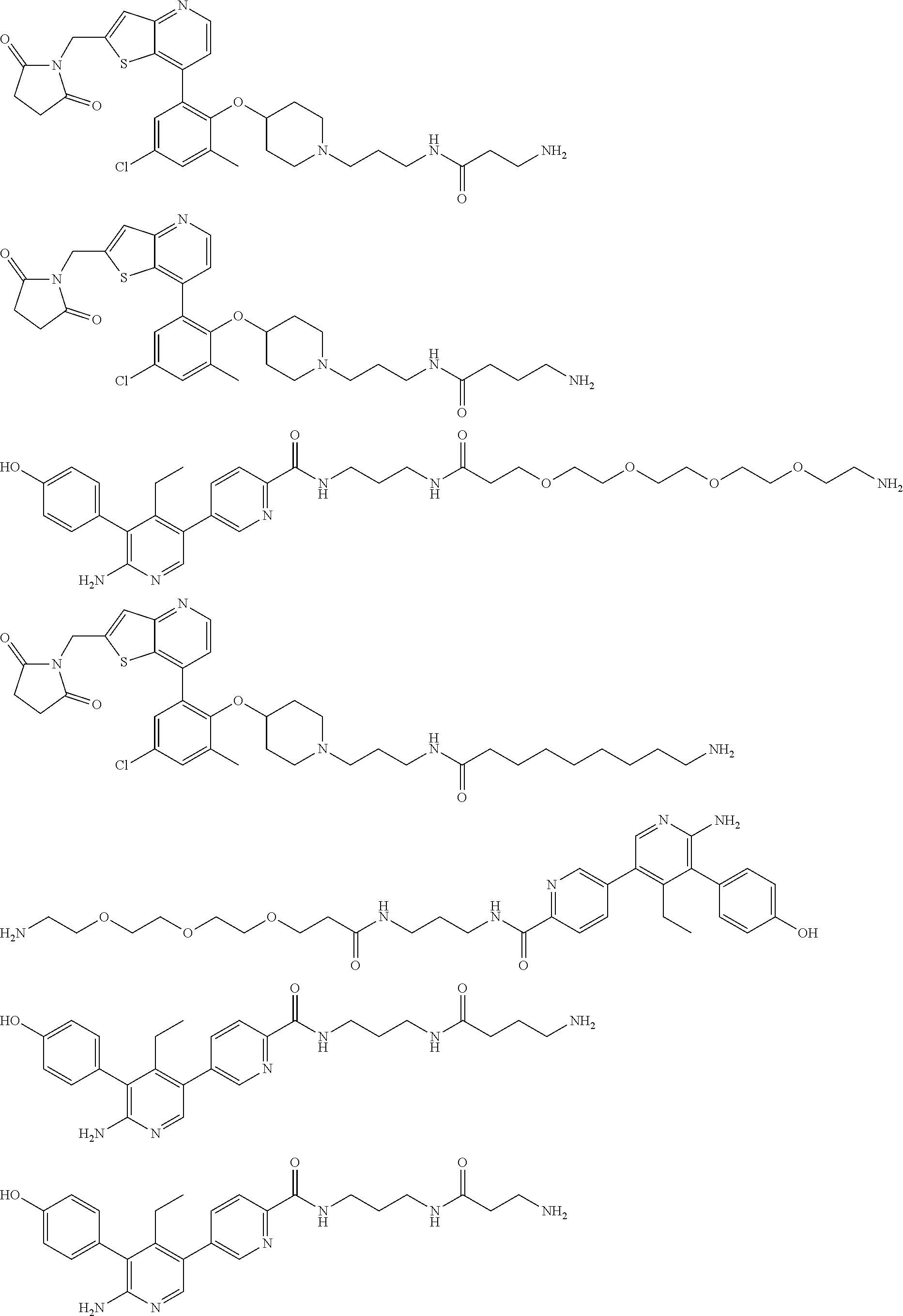

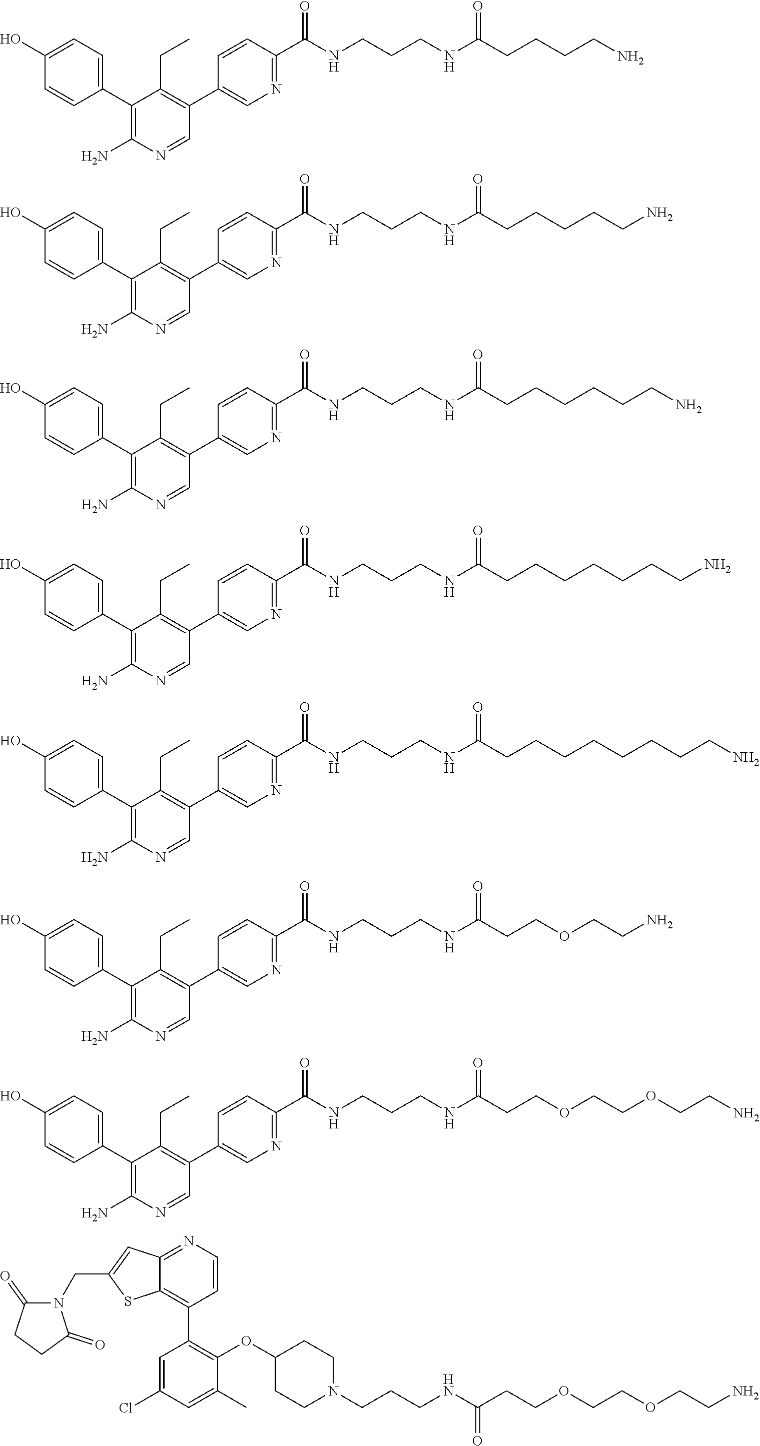

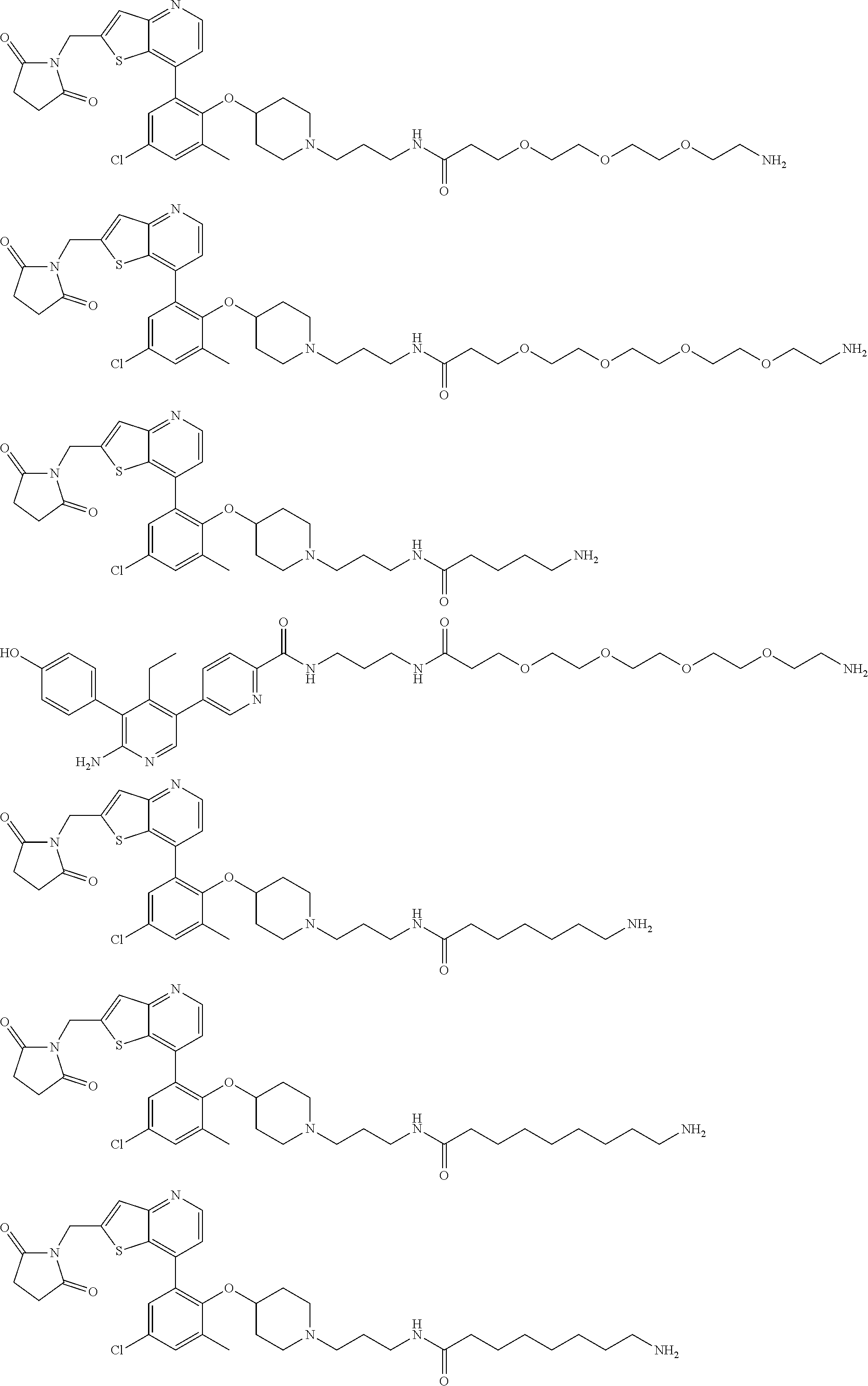

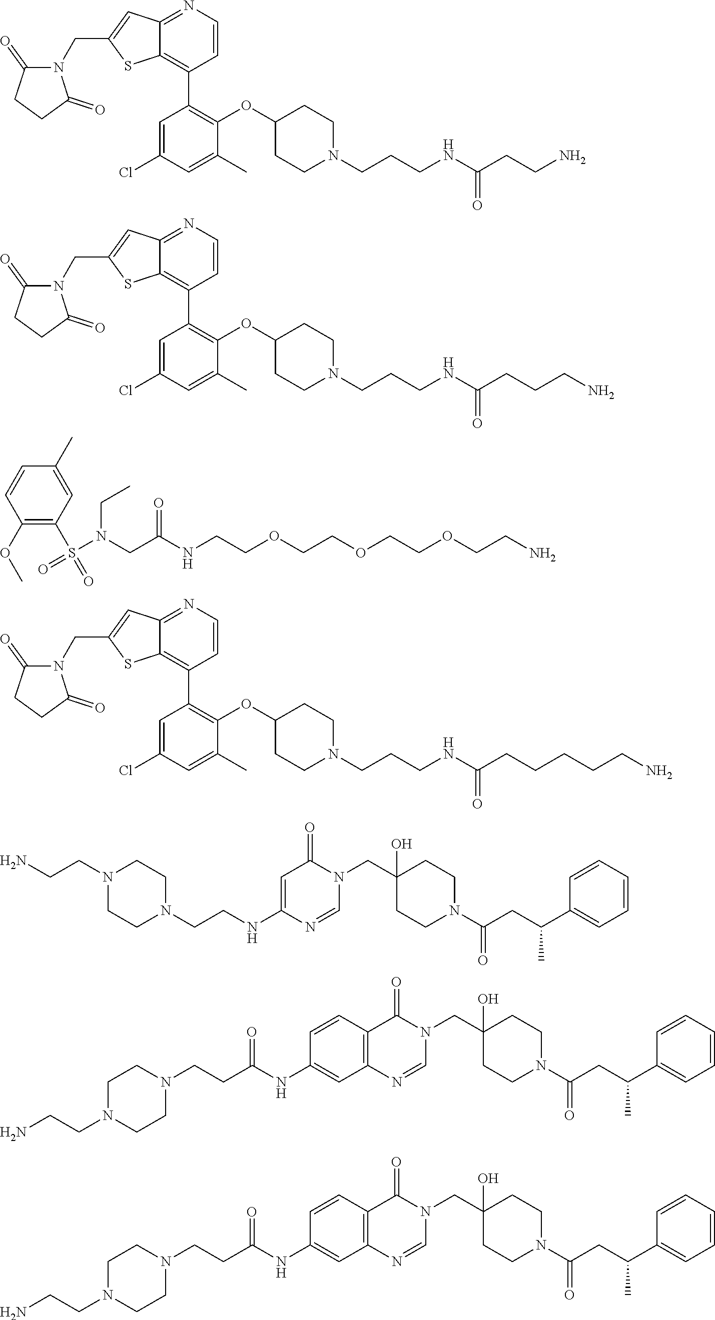

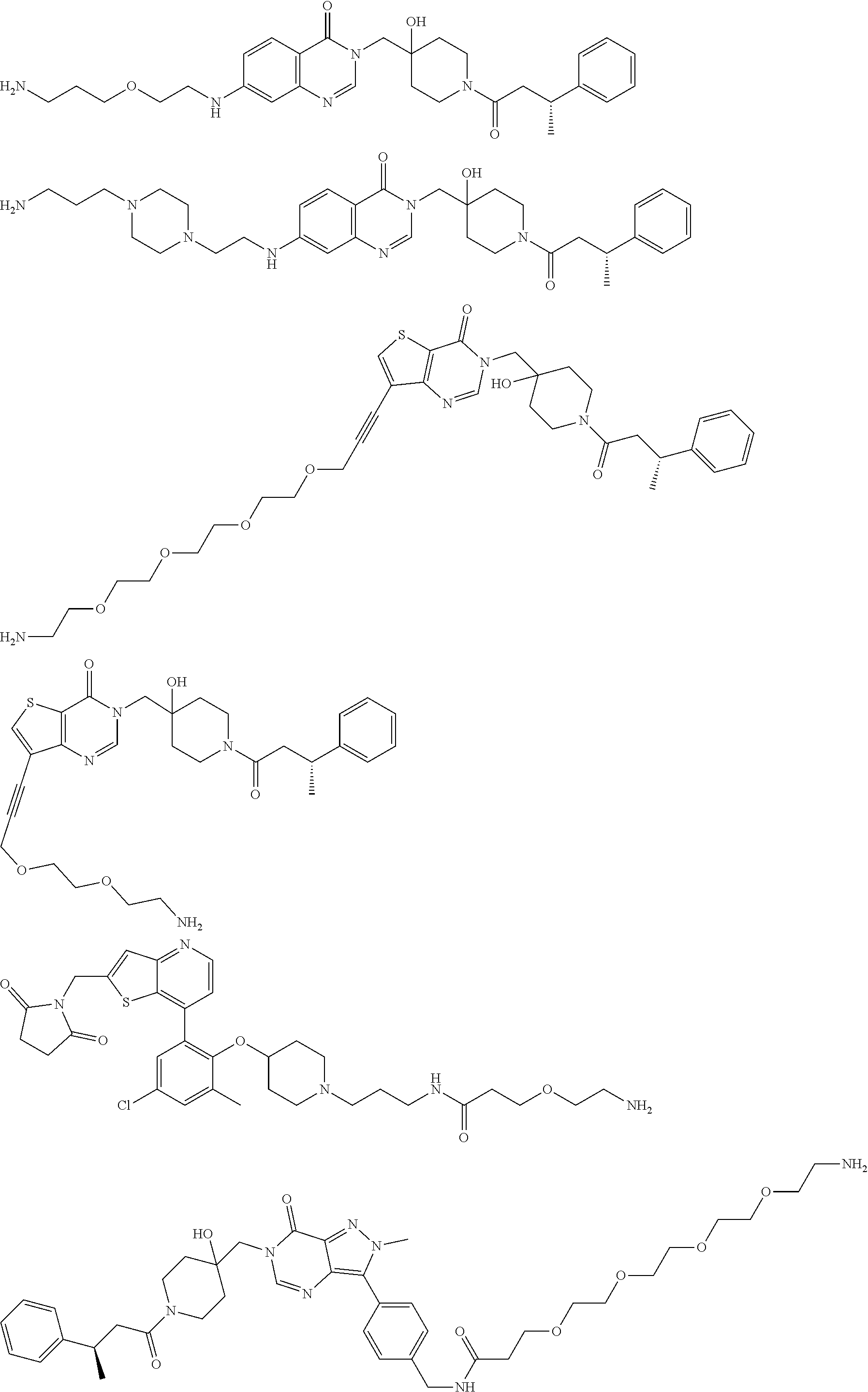

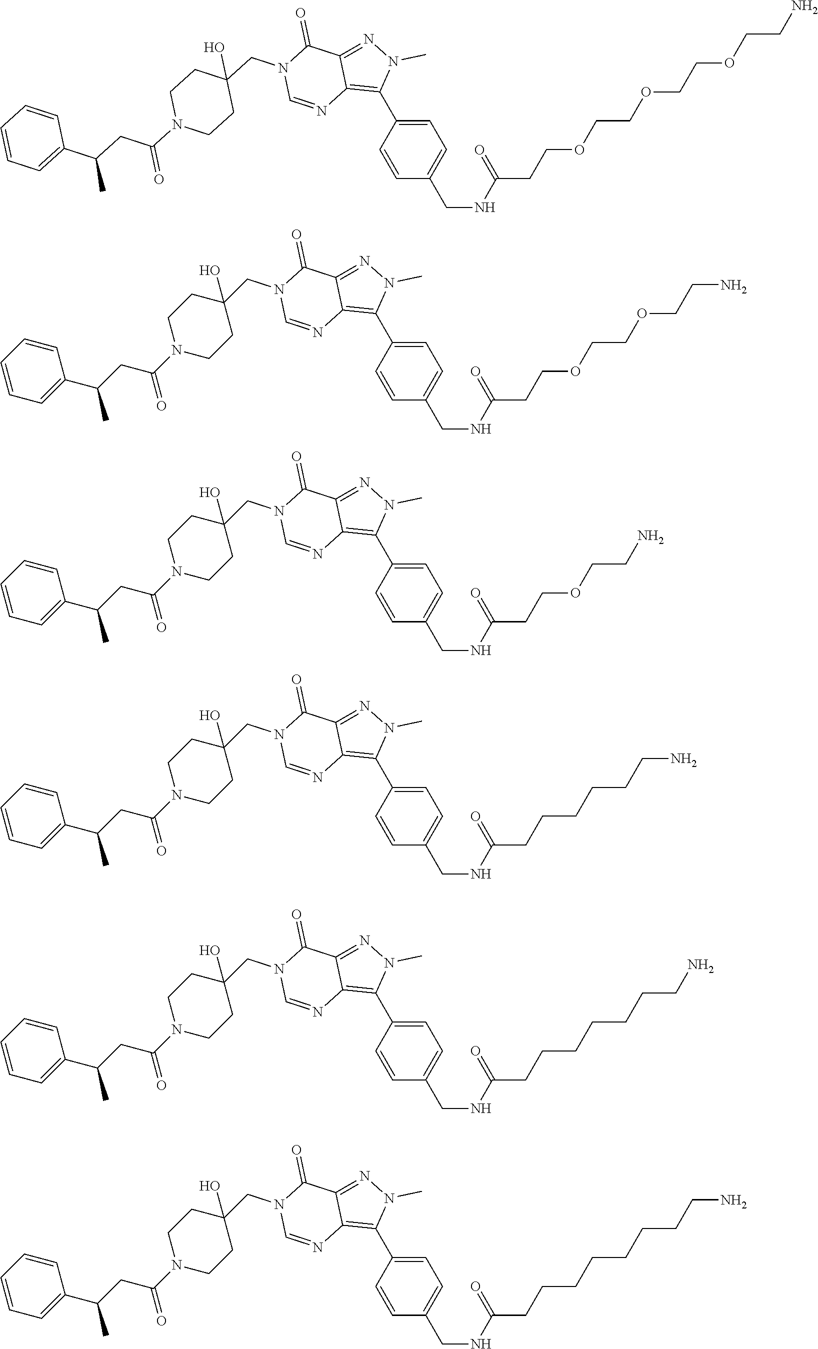

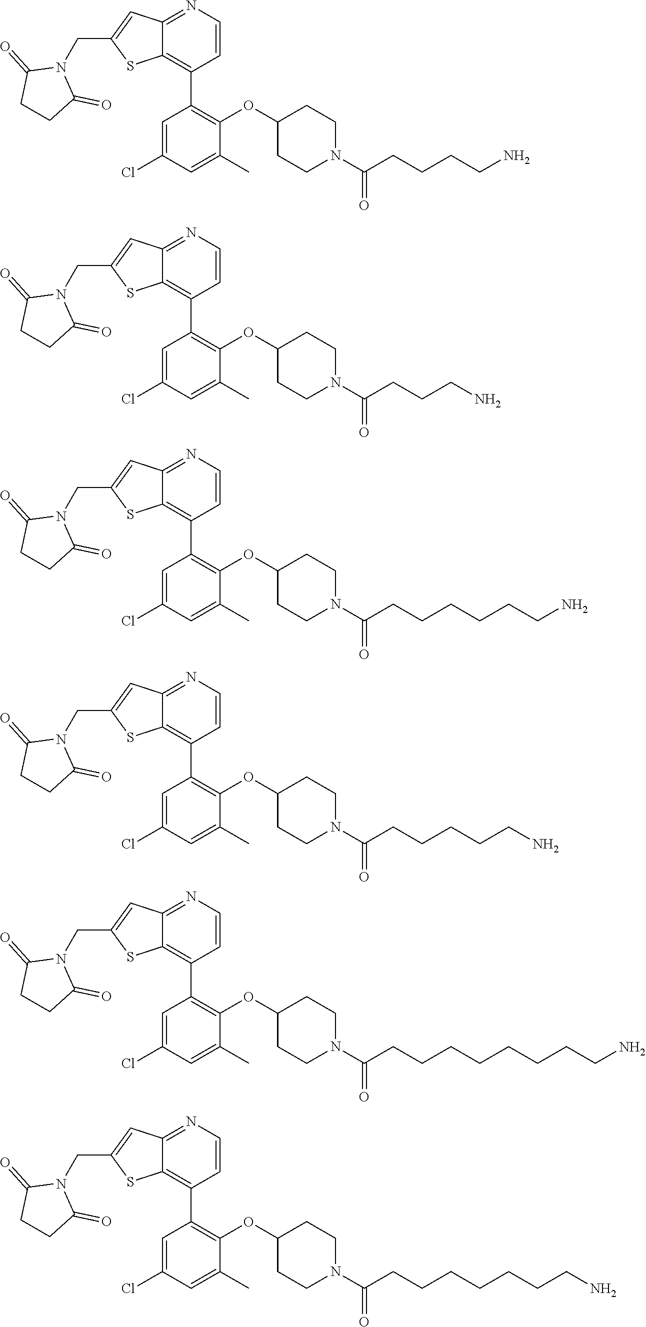 [SMILES: Cc1cc(Cl)cc(-c2ccnc3cc(CN4C(=O)CCC4=O)sc23)c1OC1CCN(C(=O)CCCCN)CC1 ; Cc1cc(Cl)cc(-c2ccnc3cc(CN4C(=O)CCC4=O)sc23)c1OC1CCN(C(=O)CCCN)CC1 ; Cc1cc(Cl)cc(-c2ccnc3cc(CN4C(=O)CCC4=O)sc23)c1OC1CCN(C(=O)CCCCCCN)CC1 ; Cc1cc(Cl)cc(-c2ccnc3cc(CN4C(=O)CCC4=O)sc23)c1OC1CCN(C(=O)CCCCCN)CC1 ; Cc1cc(Cl)cc(-c2ccnc3cc(CN4C(=O)CCC4=O)sc23)c1OC1CCN(C(=O)CCCCCCCCN)CC1 ; Cc1cc(Cl)cc(-c2ccnc3cc(CN4C(=O)CCC4=O)sc23)c1OC1CCN(C(=O)CCCCCCCN)CC1]

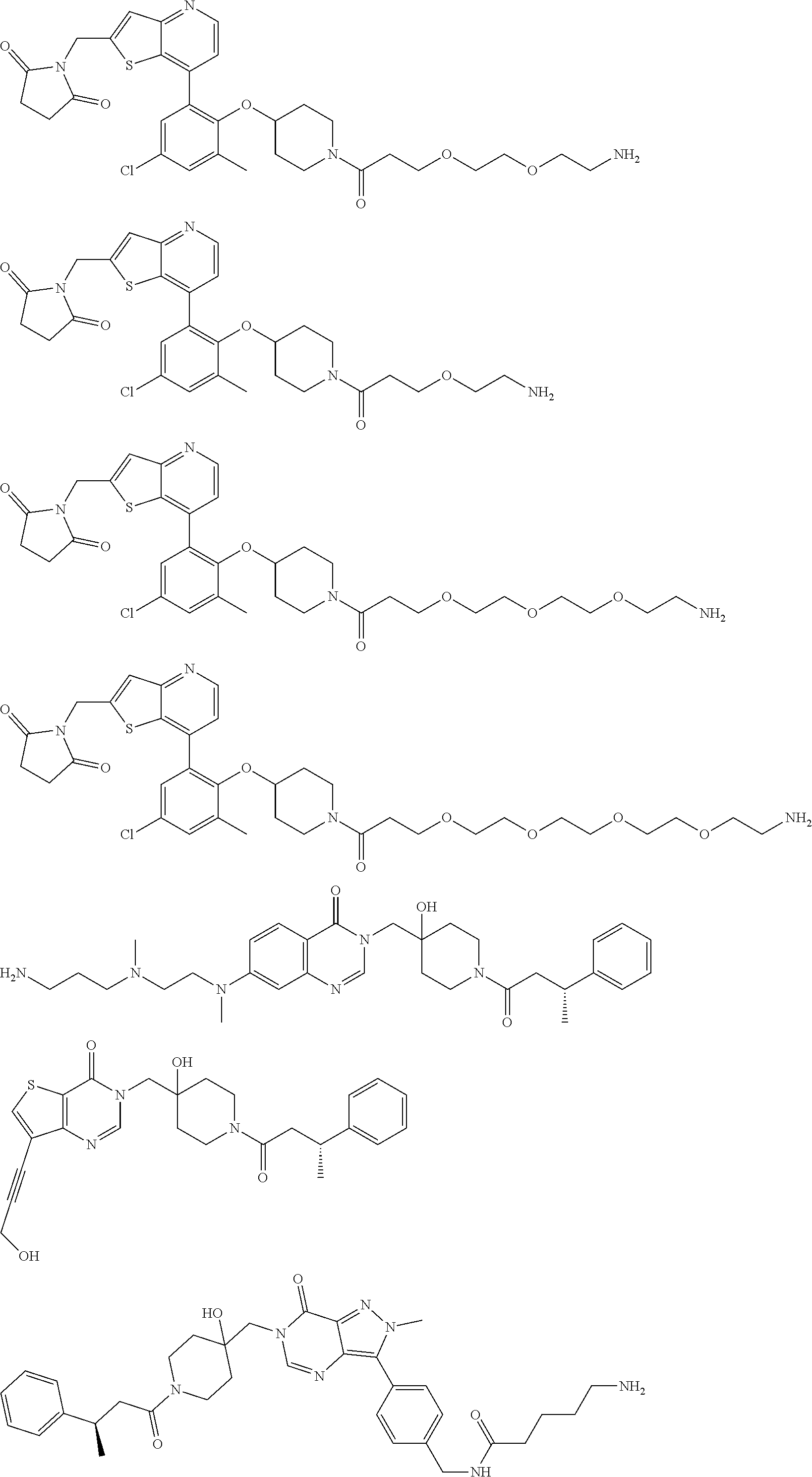

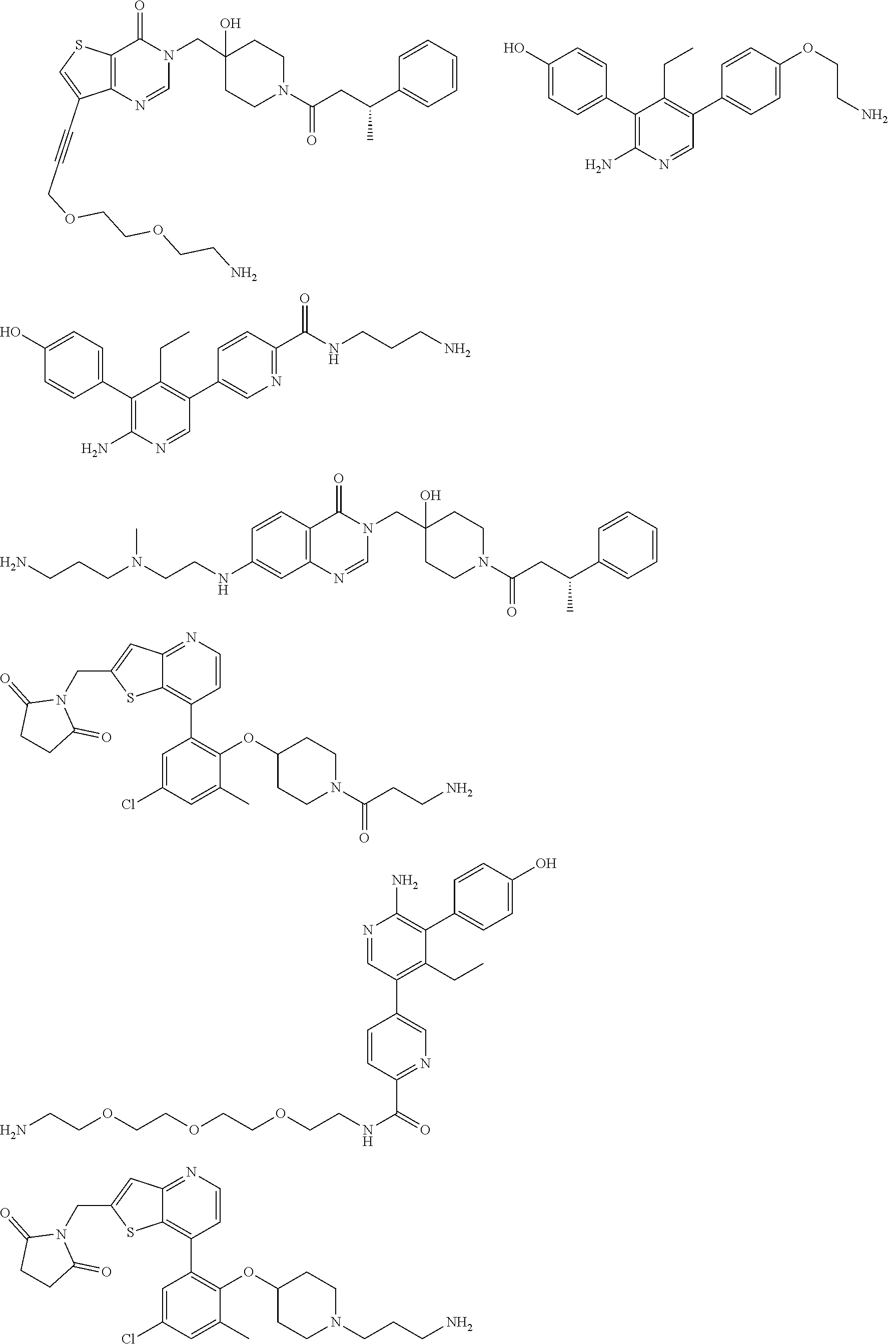

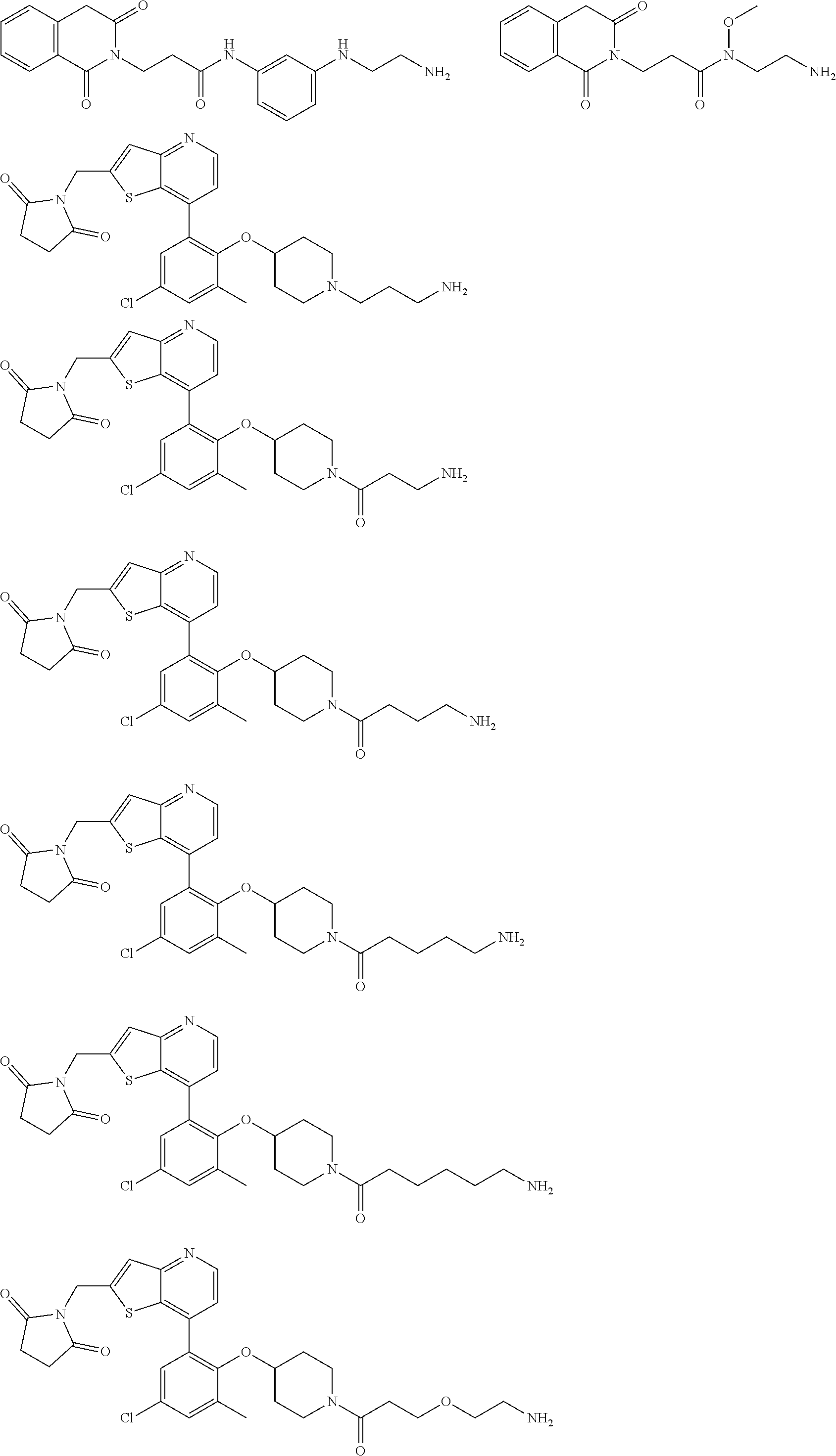

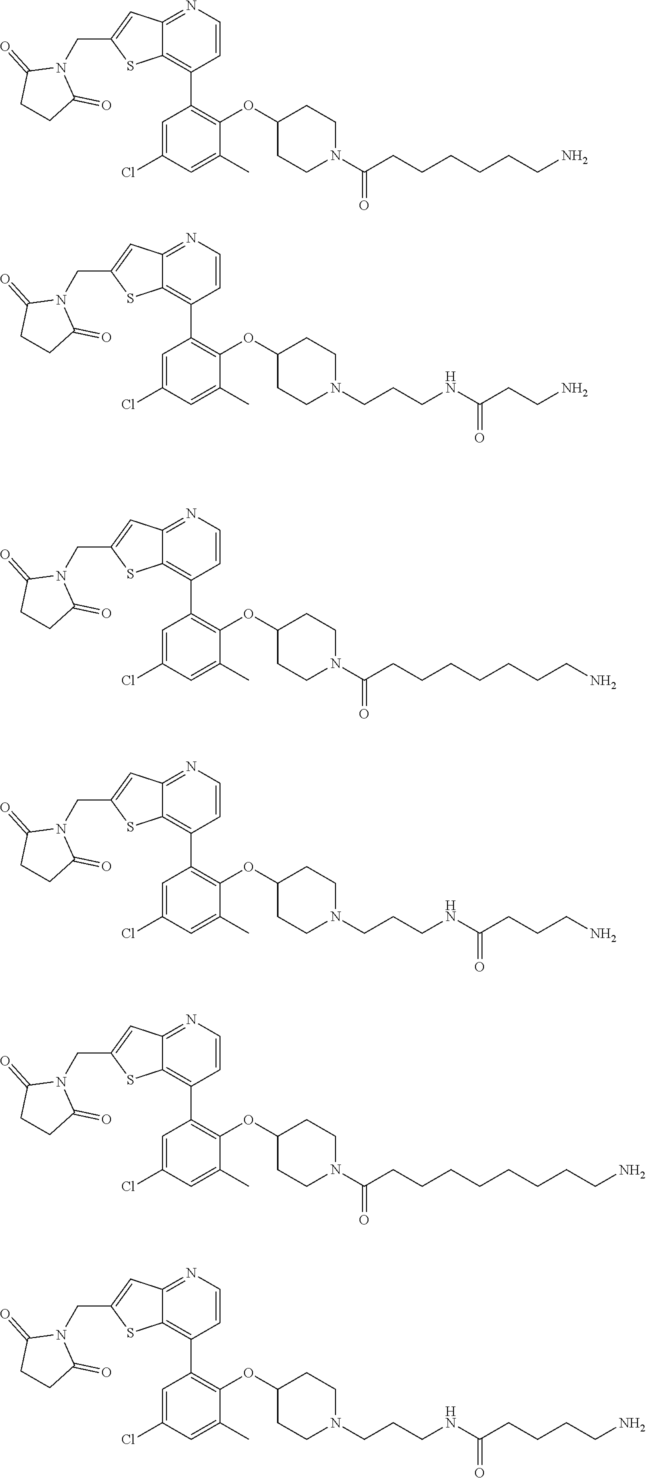 [SMILES: Cc1cc(Cl)cc(-c2ccnc3cc(CN4C(=O)CCC4=O)sc23)c1OC1CCN(C(=O)CCCCCCN)CC1 ; Cc1cc(Cl)cc(-c2ccnc3cc(CN4C(=O)CCC4=O)sc23)c1OC1CCN(CCCNC(=O)CCN)CC1 ; Cc1cc(Cl)cc(-c2ccnc3cc(CN4C(=O)CCC4=O)sc23)c1OC1CCN(C(=O)CCCCCCCN)CC1 ; Cc1cc(Cl)cc(-c2ccnc3cc(CN4C(=O)CCC4=O)sc23)c1OC1CCN(CCCNC(=O)CCCN)CC1 ; Cc1cc(Cl)cc(-c2ccnc3cc(CN4C(=O)CCC4=O)sc23)c1OC1CCN(C(=O)CCCCCCCCN)CC1 ; Cc1cc(Cl)cc(-c2ccnc3cc(CN4C(=O)CCC4=O)sc23)c1OC1CCN(CCCNC(=O)CCCCN)CC1]

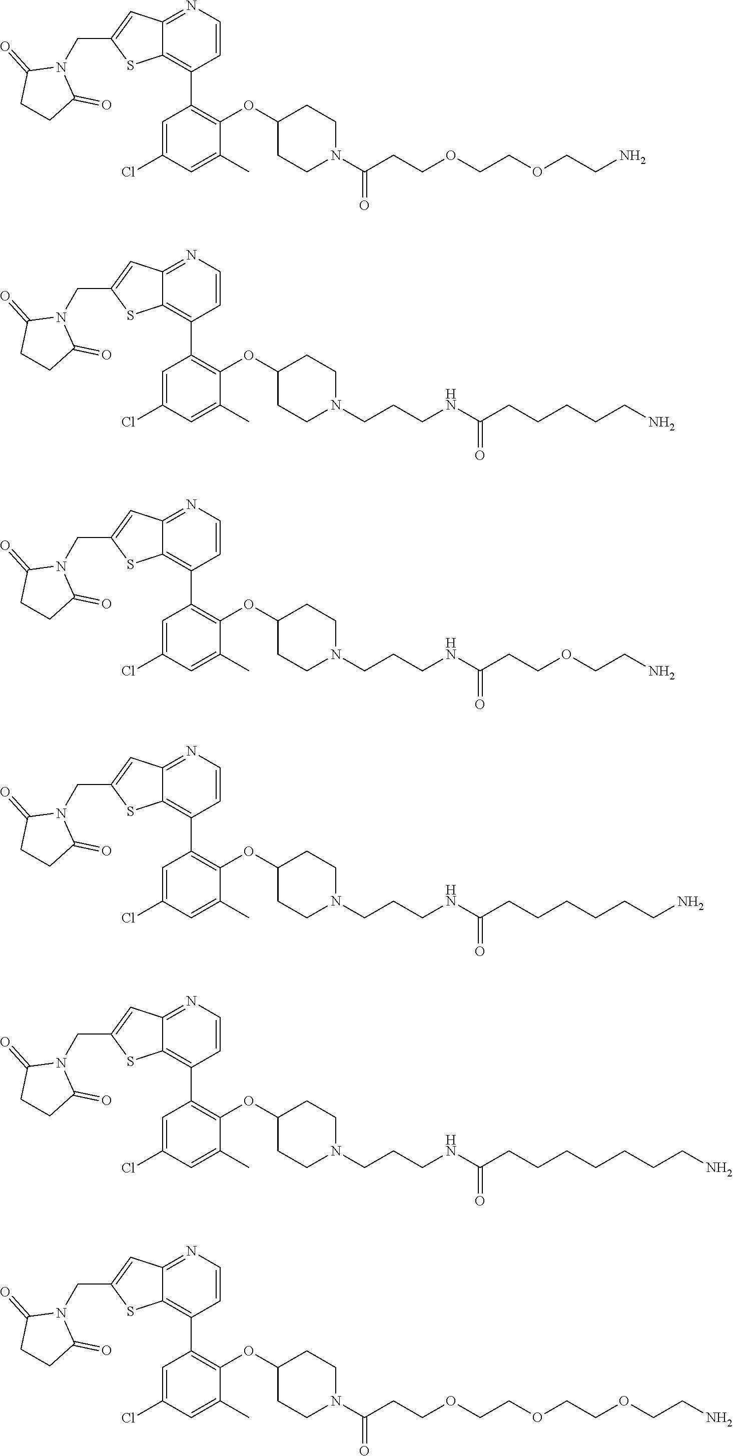 [SMILES: Cc1cc(Cl)cc(-c2ccnc3cc(CN4C(=O)CCC4=O)sc23)c1OC1CCN(C(=O)CCOCCOCCN)CC1 ; Cc1cc(Cl)cc(-c2ccnc3cc(CN4C(=O)CCC4=O)sc23)c1OC1CCN(CCCNC(=O)CCCCCN)CC1 ; Cc1cc(Cl)cc(-c2ccnc3cc(CN4C(=O)CCC4=O)sc23)c1OC1CCN(CCCNC(=O)CCOCCN)CC1 ; Cc1cc(Cl)cc(-c2ccnc3cc(CN4C(=O)CCC4=O)sc23)c1OC1CCN(CCCNC(=O)CCCCCCN)CC1 ; Cc1cc(Cl)cc(-c2ccnc3cc(CN4C(=O)CCC4=O)sc23)c1OC1CCN(CCCNC(=O)CCCCCCCN)CC1 ; Cc1cc(Cl)cc(-c2ccnc3cc(CN4C(=O)CCC4=O)sc23)c1OC1CCN(C(=O)CCOCCOCCOCCN)CC1]

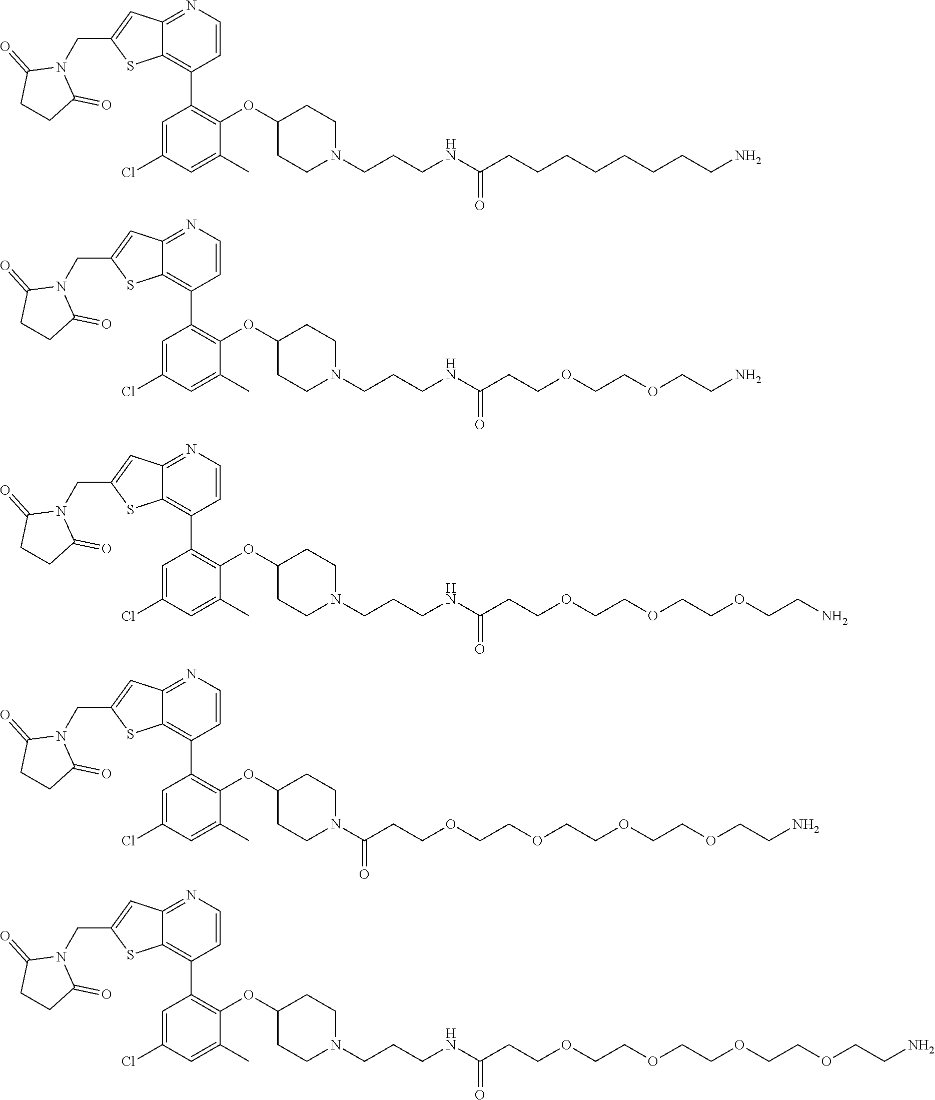 [SMILES: Cc1cc(Cl)cc(-c2ccnc3cc(CN4C(=O)CCC4=O)sc23)c1OC1CCN(CCCNC(=O)CCCCCCCCN)CC1 ; Cc1cc(Cl)cc(-c2ccnc3cc(CN4C(=O)CCC4=O)sc23)c1OC1CCN(CCCNC(=O)CCOCCOCCN)CC1 ; Cc1cc(Cl)cc(-c2ccnc3cc(CN4C(=O)CCC4=O)sc23)c1OC1CCN(CCCNC(=O)CCOCCOCCOCCN)CC1 ; Cc1cc(Cl)cc(-c2ccnc3cc(CN4C(=O)CCC4=O)sc23)c1OC1CCN(C(=O)CCOCCOCCOCCOCCN)CC1 ; Cc1cc(Cl)cc(-c2ccnc3cc(CN4C(=O)CCC4=O)sc23)c1OC1CCN(CCCNC(=O)CCOCCOCCOCCOCCN)CC1]

or a pharmaceutically acceptable salt thereof, wherein each of the above USP7 Targeting Ligand-Linker is substituted by 1 Ubiquitinated Protein Targeting Ligand or LinkerB-Ubiquitinated Protein Targeting Ligand and 0, 1, 2, or 3 R¹⁰² substituents; and
R¹⁰² substituents are independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹1C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹.

### Protein Stabilizing Compounds

Non-limiting examples of CFTR stabilizing compounds of the present invention include:

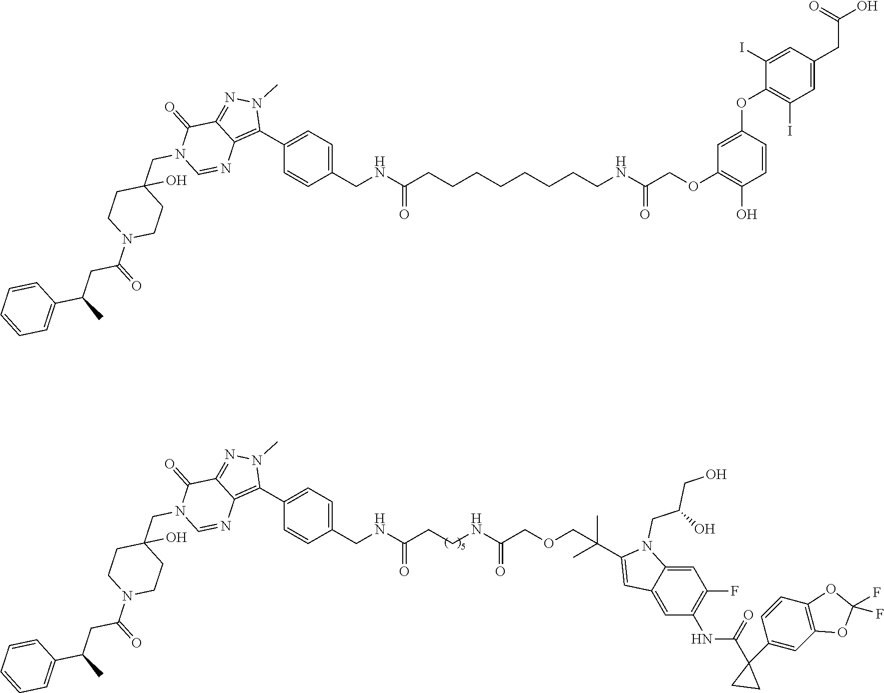

or a pharmaceutically acceptable salt thereof.

In certain embodiments the CFTR Targeting Ligand-Linker is selected from:

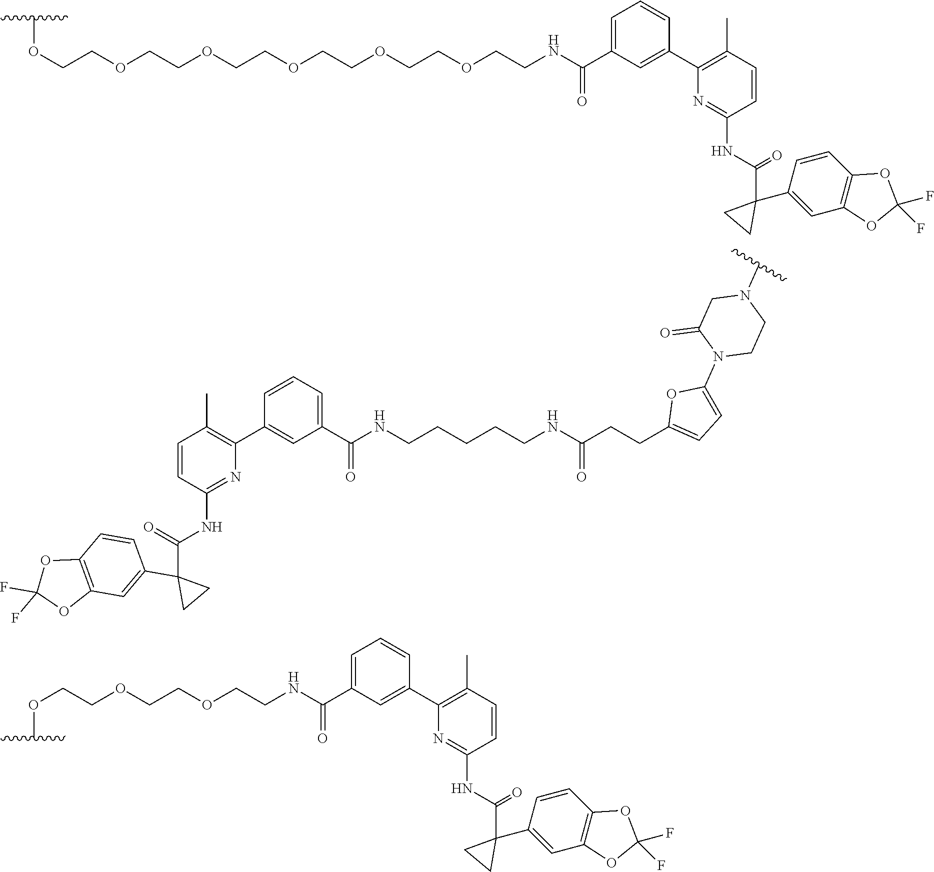

Non-limiting examples of phenylalanine hydroxylase (PAH) stabilizing compounds of the present invention include:

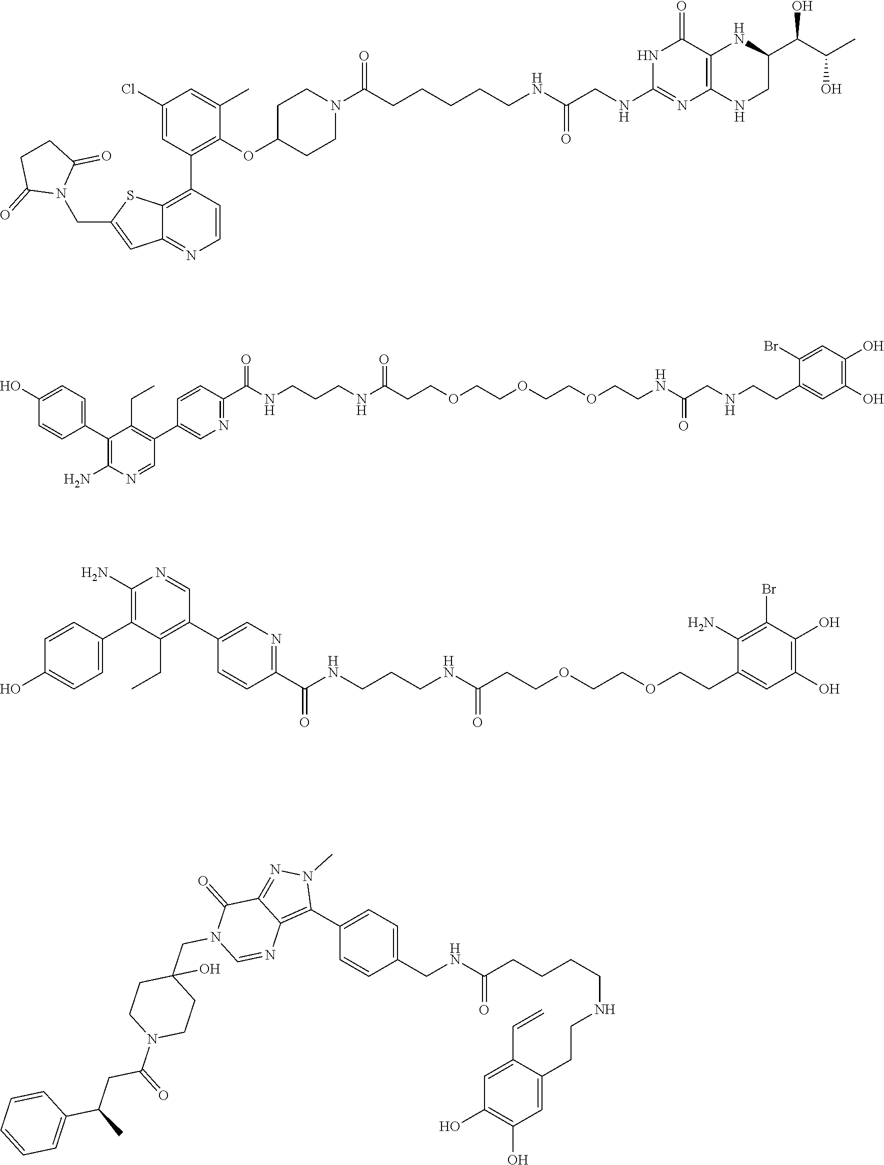 [SMILES: Cc1cc(Cl)cc(-c2ccnc3cc(CN4C(=O)CCC4=O)sc23)c1OC1CCN(C(=O)CCCCCNC(=O)CNc2nc3c(c(=O)[nH]2)N[C@@H]([C@@H](O)[C@H](C)O)CN3)CC1 ; CCc1c(-c2ccc(C(=O)NCCCNC(=O)CCOCCOCCOCCNC(=O)CNCCc3cc(O)c(O)cc3Br)nc2)cnc(N)c1-c1ccc(O)cc1 ; CCc1c(-c2ccc(C(=O)NCCCNC(=O)CCOCCOCCc3cc(O)c(O)c(Br)c3N)nc2)cnc(N)c1-c1ccc(O)cc1 ; C=Cc1cc(O)c(O)cc1CCNCCCCC(=O)NCc1ccc(-c2c3ncn(CC4(O)CCN(C(=O)C[C@@H](C)c5ccccc5)CC4)c(=O)c3nn2C)cc1]

or a pharmaceutically acceptable salt thereof.

Non-limiting examples of tumor protein p53, MDM2, or P53 MDM2 complex stabilizing compounds of the present invention include:

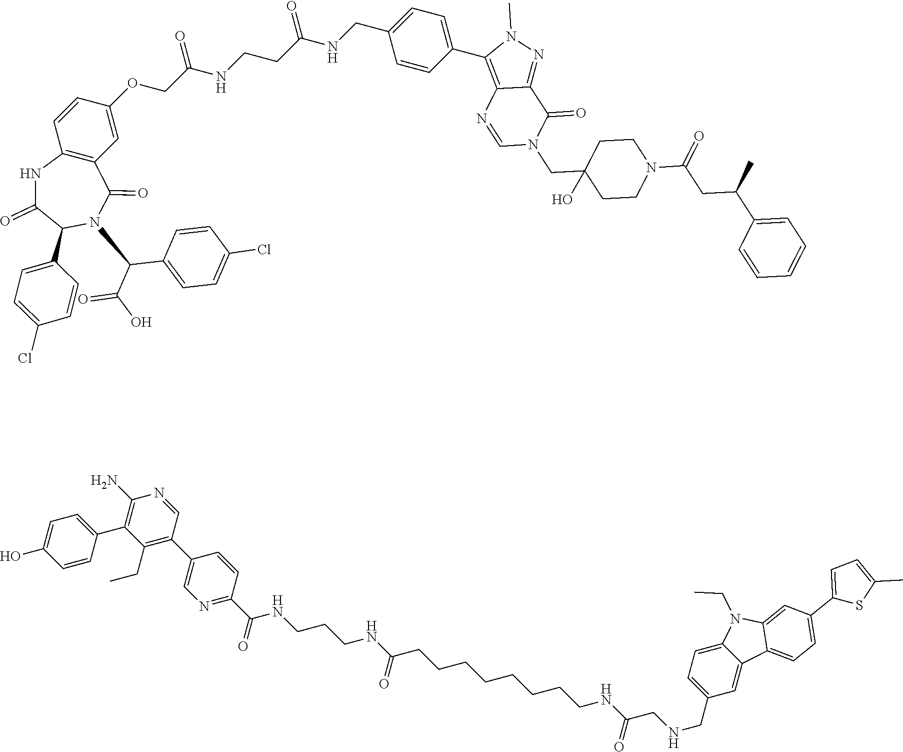 [SMILES: C[C@H](CC(=O)N1CCC(O)(Cn2cnc3c(-c4ccc(CNC(=O)CCNC(=O)COc5ccc6c(c5)C(=O)N([C@H](C(=O)O)c5ccc(Cl)cc5)[C@@H](c5ccc(Cl)cc5)C(=O)N6)cc4)n(C)nc3c2=O)CC1)c1ccccc1 ; CCc1c(-c2ccc(C(=O)NCCCNC(=O)CCCCCCCCNC(=O)CNCc3ccc4c(c3)c3ccc(-c5ccc(C)s5)cc3n4CC)nc2)cnc(N)c1-c1ccc(O)cc1]

or a pharmaceutically acceptable salt thereof.

Non-limiting examples of rhodopsin stabilizing compounds of the present invention include:

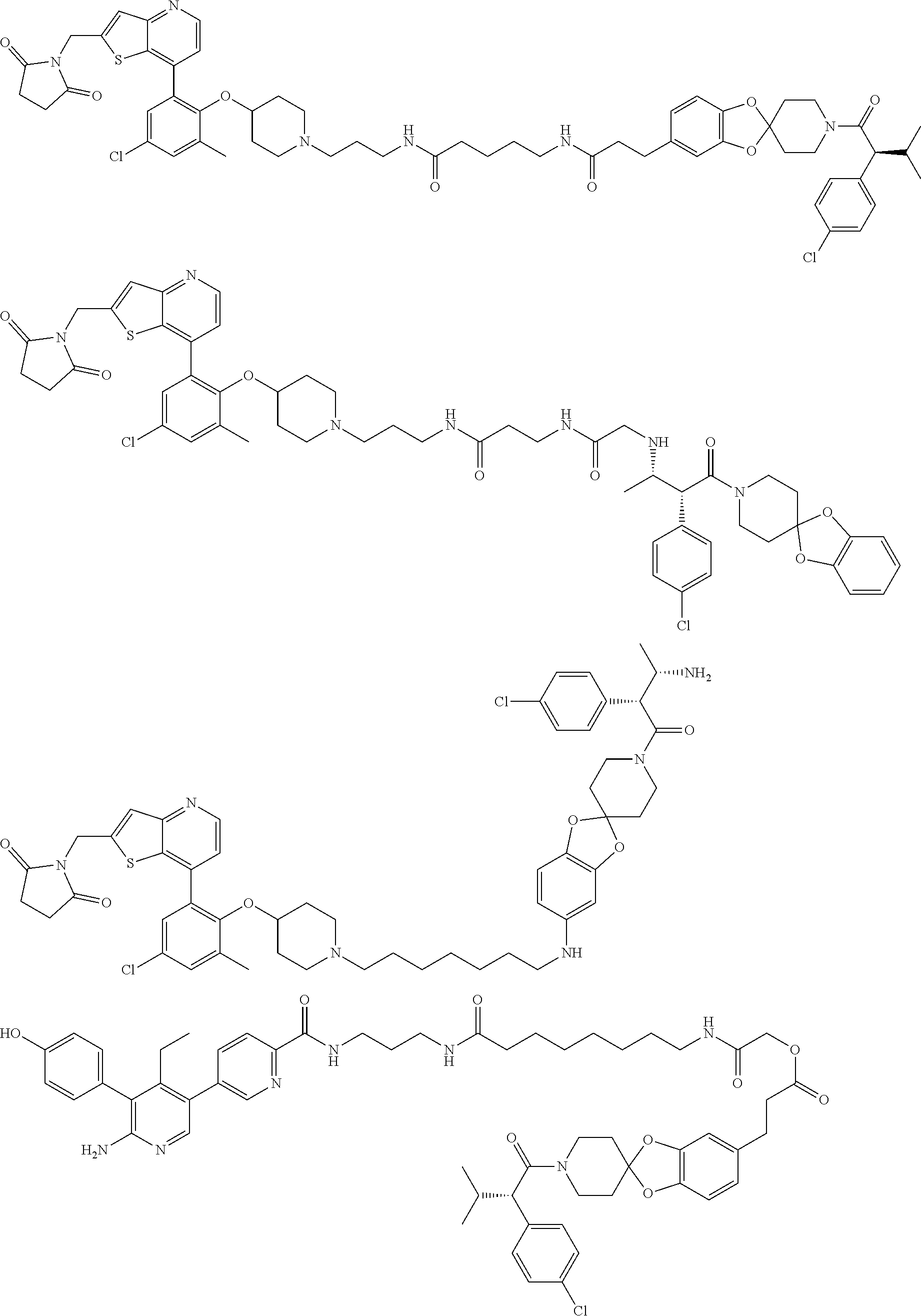

or a pharmaceutically acceptable salt thereof.

Non-limiting examples of c-myc stabilizing compounds of the present invention include:

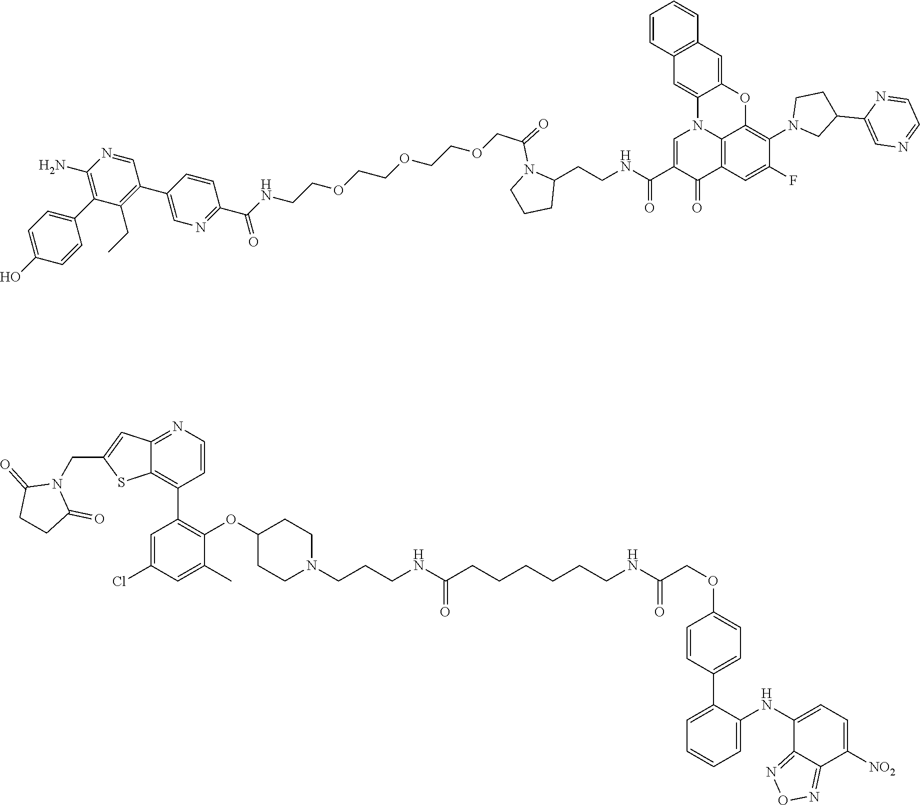 [SMILES: CCc1c(-c2ccc(C(=O)NCCOCCOCCOCC(=O)N3CCCC3CCNC(=O)c3cn4c5c(c(N6CCC(c7cnccn7)C6)c(F)cc5c3=O)Oc3cc5ccccc5cc3-4)nc2)cnc(N)c1-c1ccc(O)cc1 ; Cc1cc(Cl)cc(-c2ccnc3cc(CN4C(=O)CCC4=O)sc23)c1OC1CCN(CCCNC(=O)CCCCCCNC(=O)COc2ccc(-c3ccccc3Nc3ccc([N+](=O)[O-])c4nonc34)cc2)CC1]

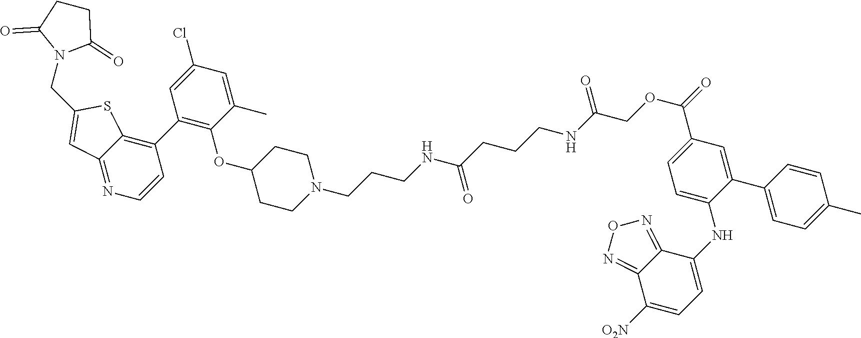 [SMILES: Cc1ccc(-c2cc(C(=O)OCC(=O)NCCCC(=O)NCCCN3CCC(Oc4c(C)cc(Cl)cc4-c4ccnc5cc(CN6C(=O)CCC6=O)sc45)CC3)ccc2Nc2ccc([N+](=O)[O-])c3nonc23)cc1]

or a pharmaceutically acceptable salt thereof.

Non-limiting examples of receptor interacting protein kinase 1 (RIPK1) stabilizing compounds of the present invention include:

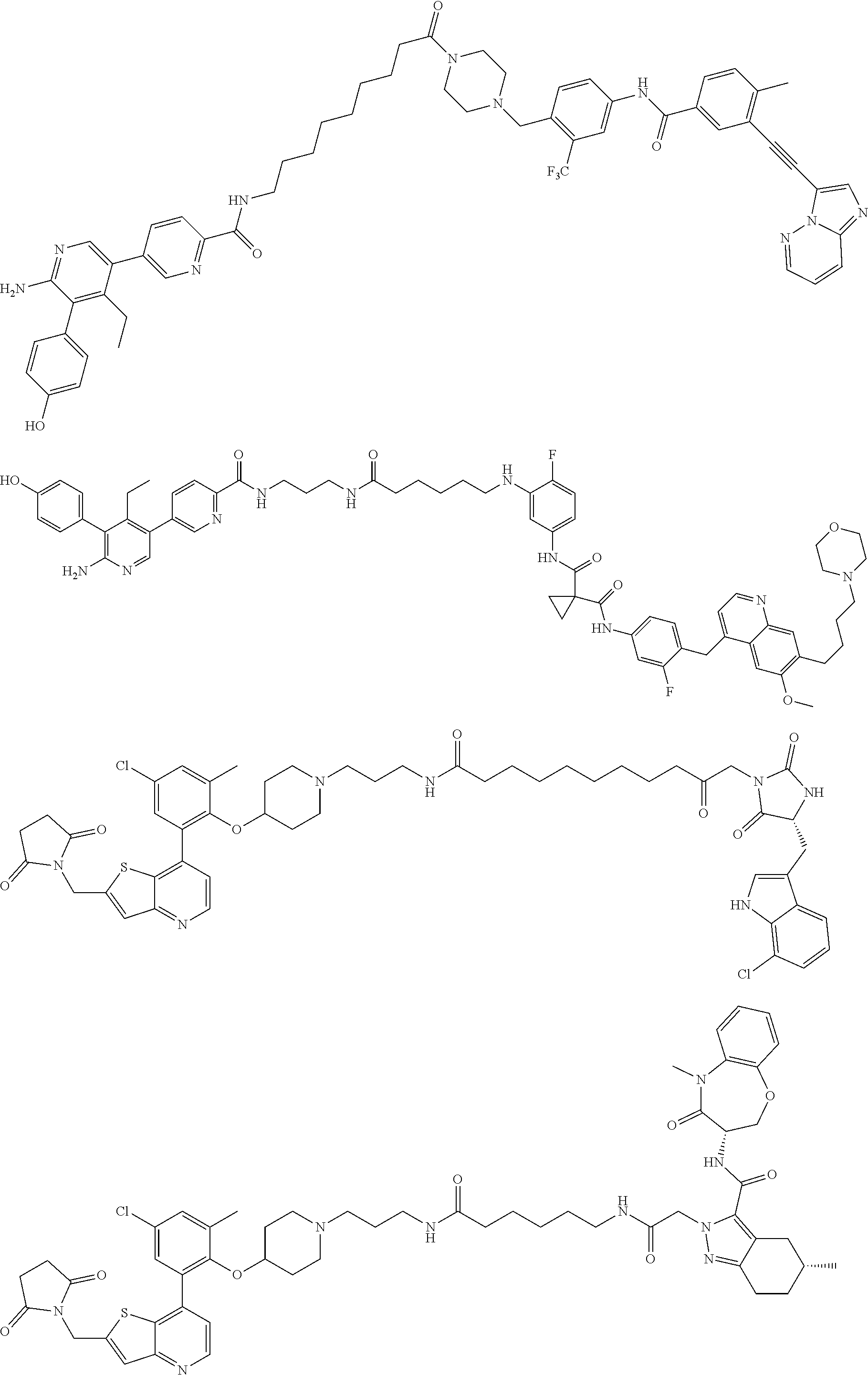

or a pharmaceutically acceptable salt thereof.

Non-limiting examples of MSH2 stabilizing compounds of the present invention include:

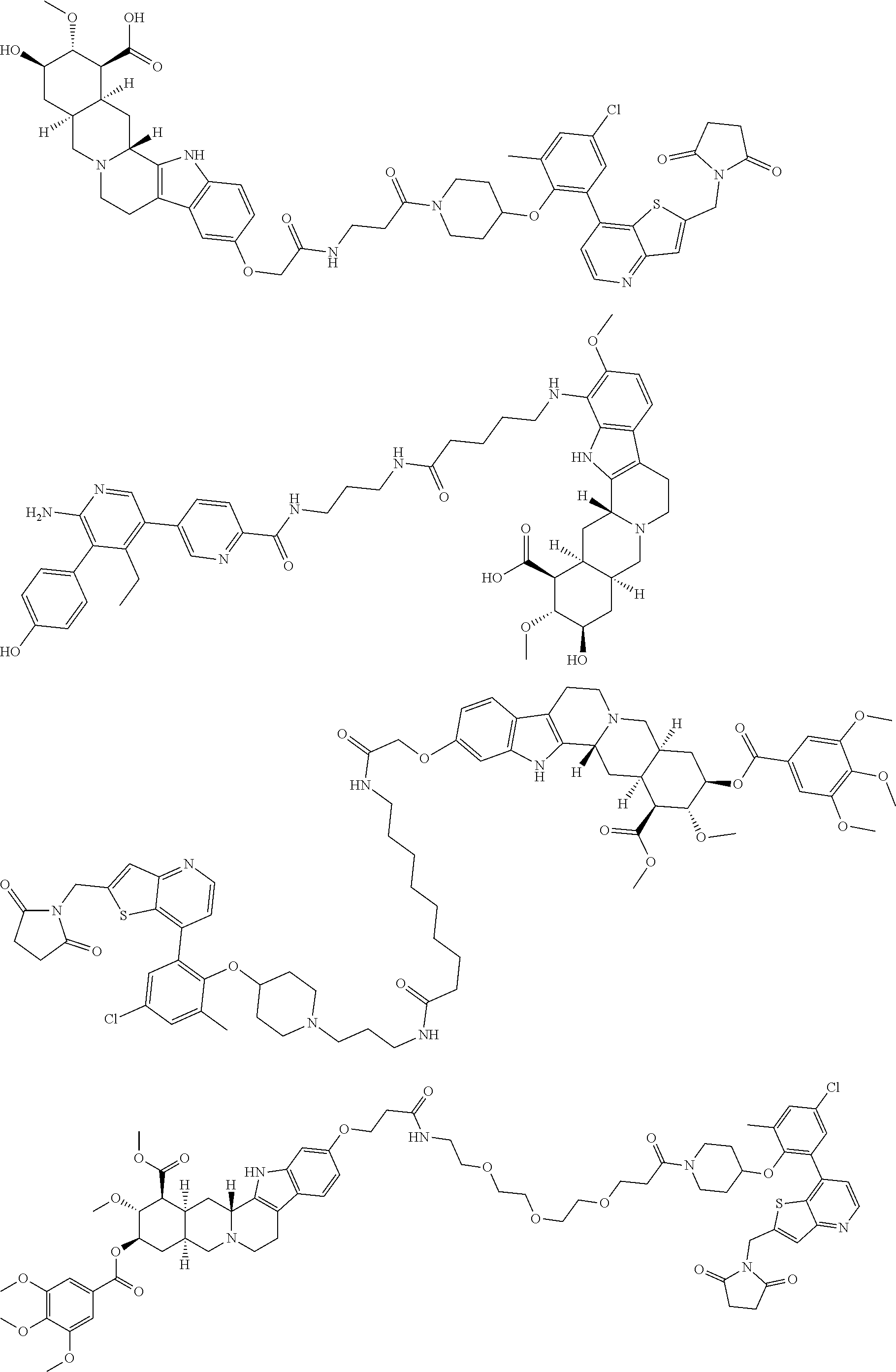

or a pharmaceutically acceptable salt thereof.

Non-limiting examples of p27Kip1 stabilizing compounds of the present invention include:

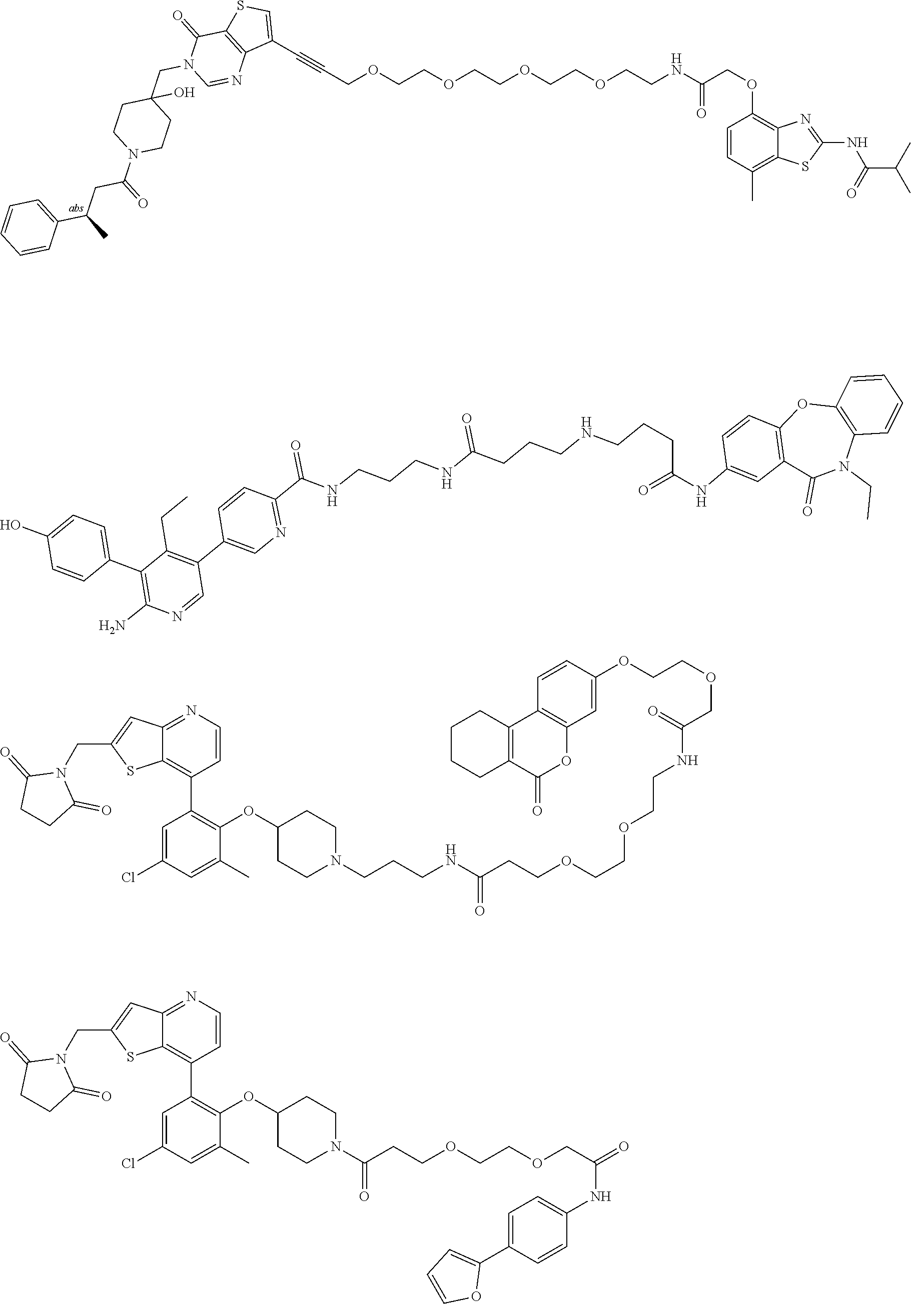 [SMILES: Cc1ccc(OCC(=O)NCCOCCOCCOCCOCC#Cc2csc3c(=O)n(CC4(O)CCN(C(=O)C[C@@H](C)c5ccccc5)CC4)cnc23)c2nc(NC(=O)C(C)C)sc12 ; CCc1c(-c2ccc(C(=O)NCCCNC(=O)CCCNCCCC(=O)Nc3ccc4c(c3)C(=O)N(CC)c3ccccc3O4)nc2)cnc(N)c1-c1ccc(O)cc1 ; Cc1cc(Cl)cc(-c2ccnc3cc(CN4C(=O)CCC4=O)sc23)c1OC1CCN(CCCNC(=O)CCOCCOCCNC(=O)COCCOc2ccc3c4c(c(=O)oc3c2)CCCC4)CC1 ; Cc1cc(Cl)cc(-c2ccnc3cc(CN4C(=O)CCC4=O)sc23)c1OC1CCN(C(=O)CCOCCOCC(=O)Nc2ccc(-c3ccco3)cc2)CC1]

or a pharmaceutically acceptable salt thereof.

Non-limiting examples of ABCA4 stabilizing compounds of the present invention include:

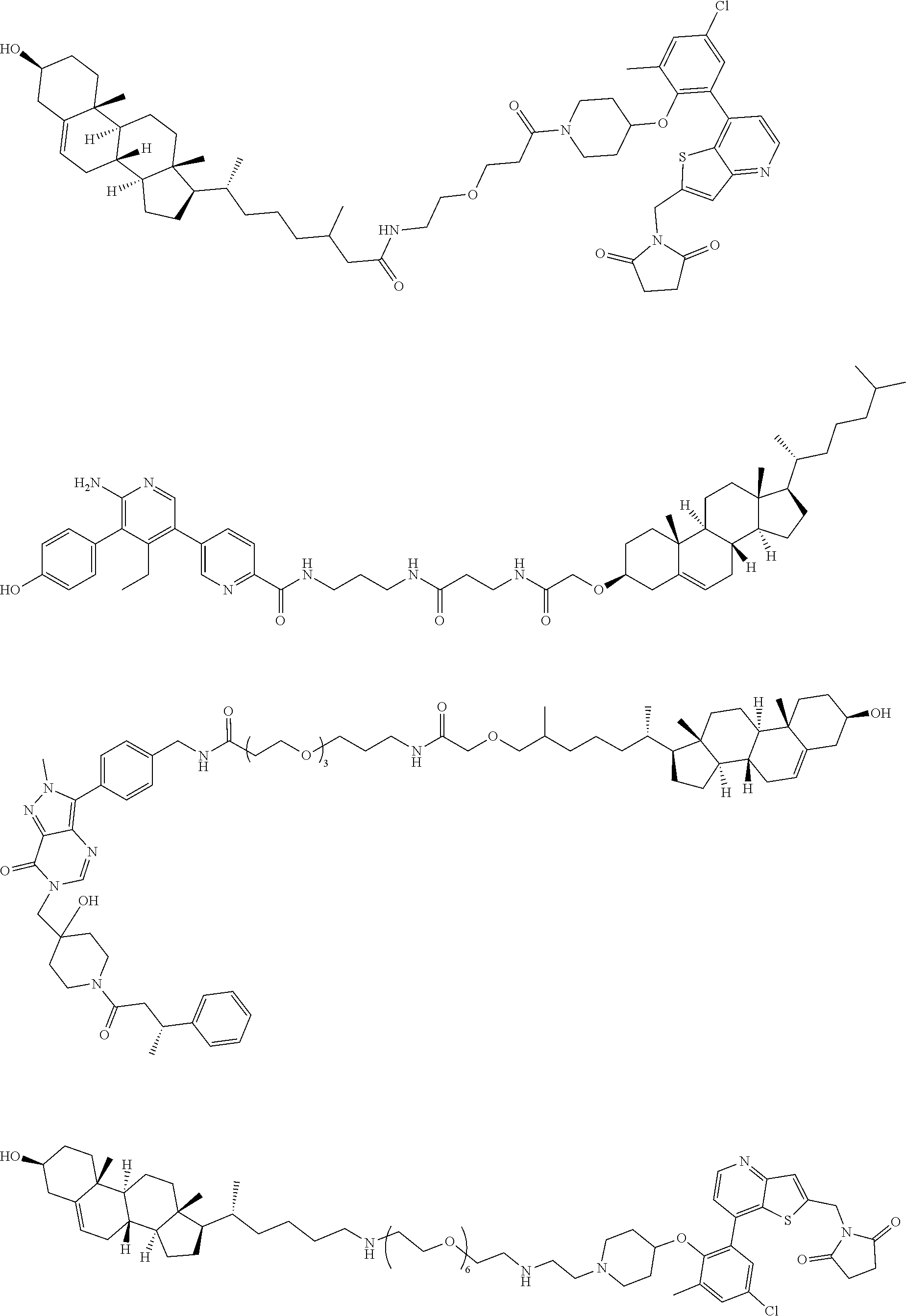

or a pharmaceutically acceptable salt thereof.

Non-limiting examples of ABCB11 stabilizing compounds of the present invention include:

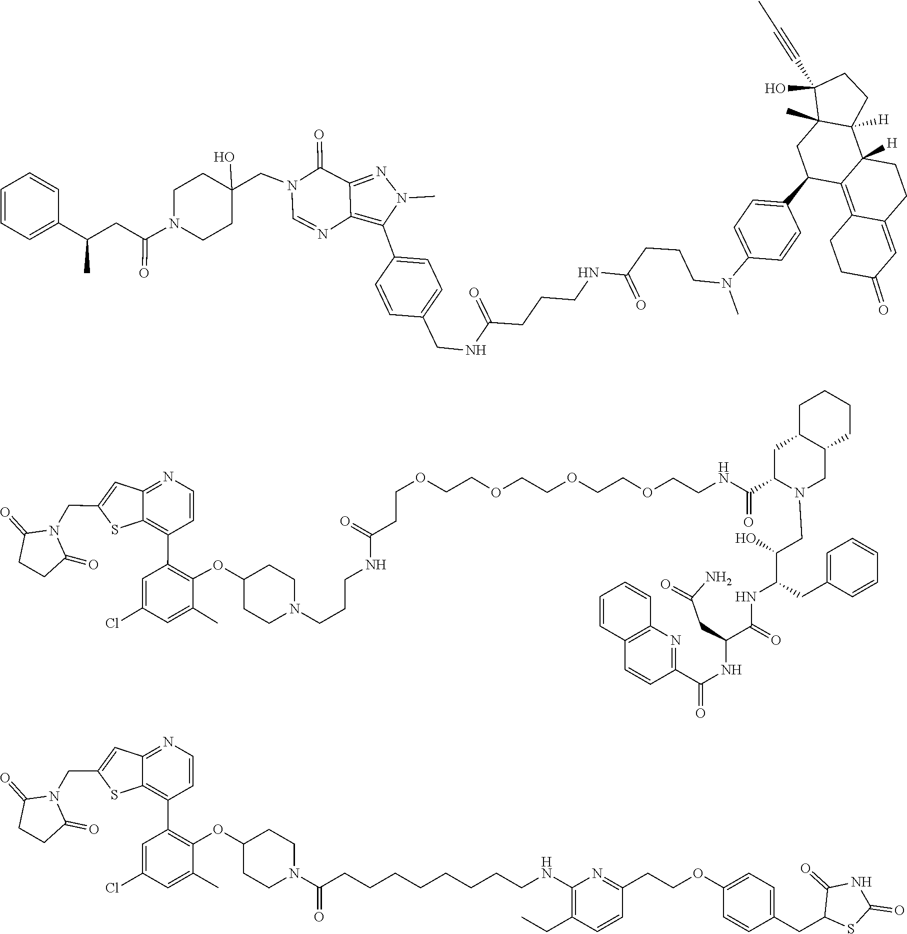 [SMILES: CC#C[C@]1(O)CC[C@H]2[C@@H]3CCC4=CC(=O)CCC4=C3[C@@H](c3ccc(N(C)CCCC(=O)NCCCC(=O)NCc4ccc(-c5c6ncn(CC7(O)CCN(C(=O)C[C@@H](C)c8ccccc8)CC7)c(=O)c6nn5C)cc4)cc3)C[C@@]21C ; Cc1cc(Cl)cc(-c2ccnc3cc(CN4C(=O)CCC4=O)sc23)c1OC1CCN(CCCNC(=O)CCOCCOCCOCCOCCNC(=O)[C@@H]2C[C@@H]3CCCC[C@@H]3CN2C[C@@H](O)[C@H](Cc2ccccc2)NC(=O)[C@H](CC(N)=O)NC(=O)c2ccc3ccccc3n2)CC1 ; CCc1ccc(CCOc2ccc(CC3SC(=O)NC3=O)cc2)nc1NCCCCCCCCC(=O)N1CCC(Oc2c(C)cc(Cl)cc2-c2ccnc3cc(CN4C(=O)CCC4=O)sc23)CC1]

or a pharmaceutically acceptable salt thereof.

Non-limiting examples of ChAT stabilizing compounds of the present invention include:

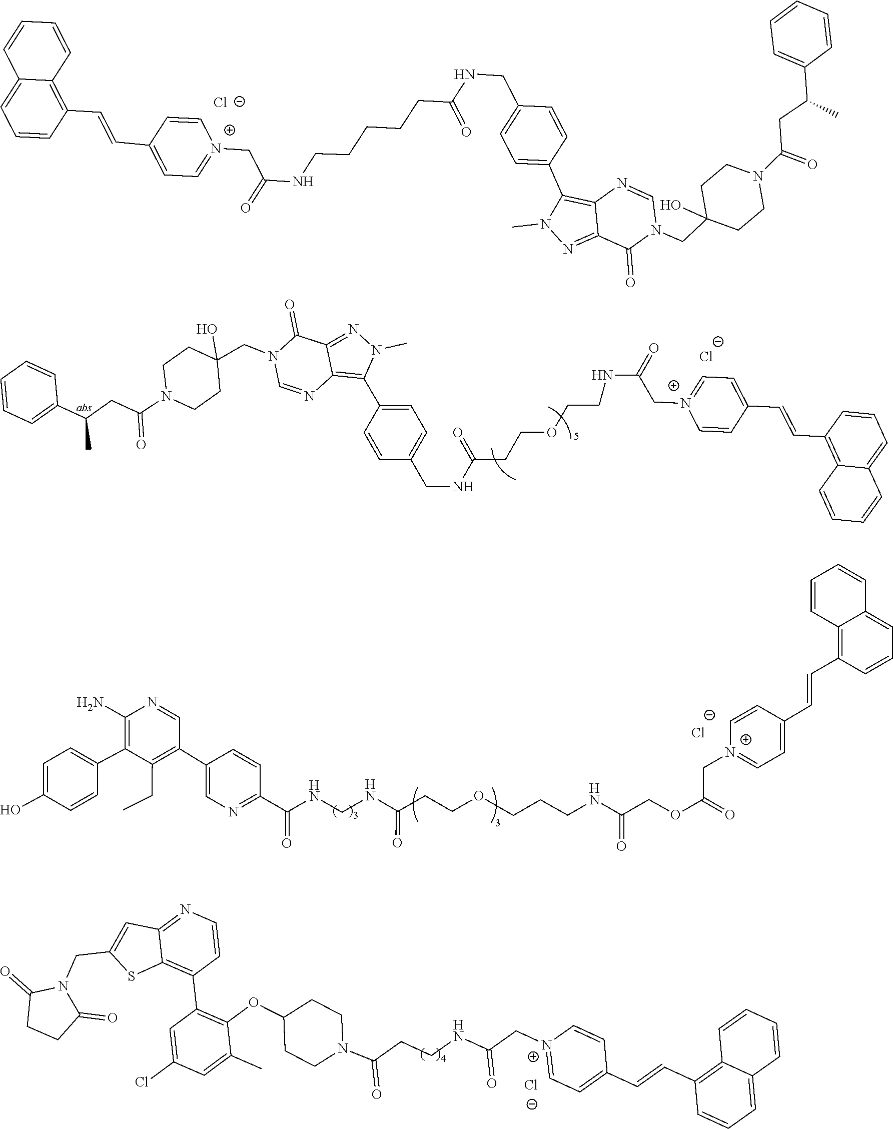

or a pharmaceutically acceptable salt thereof.

Non-limiting examples of CYLD stabilizing compounds of the present invention include:

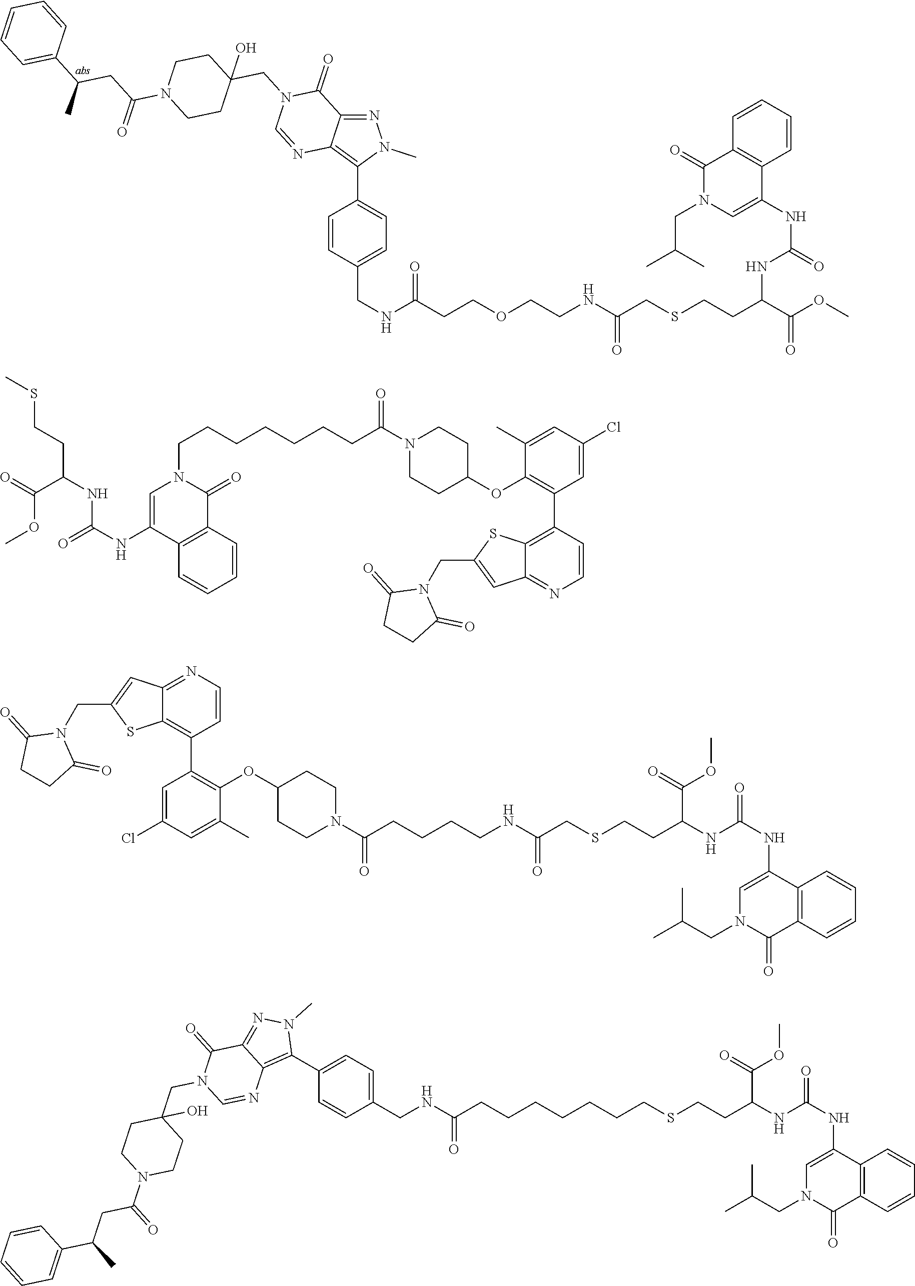 [SMILES: COC(=O)C(CCSCC(=O)NCCOCCC(=O)NCc1ccc(-c2c3ncn(CC4(O)CCN(C(=O)C[C@@H](C)c5ccccc5)CC4)c(=O)c3nn2C)cc1)NC(=O)Nc1cn(CC(C)C)c(=O)c2ccccc12 ; COC(=O)C(CCSC)NC(=O)Nc1cn(CCCCCCCC(=O)N2CCC(Oc3c(C)cc(Cl)cc3-c3ccnc4cc(CN5C(=O)CCC5=O)sc34)CC2)c(=O)c2ccccc12 ; COC(=O)C(CCSCC(=O)NCCCCC(=O)N1CCC(Oc2c(C)cc(Cl)cc2-c2ccnc3cc(CN4C(=O)CCC4=O)sc23)CC1)NC(=O)Nc1cn(CC(C)C)c(=O)c2ccccc12 ; COC(=O)C(CCSCCCCCCCC(=O)NCc1ccc(-c2c3ncn(CC4(O)CCN(C(=O)C[C@@H](C)c5ccccc5)CC4)c(=O)c3nn2C)cc1)NC(=O)Nc1cn(CC(C)C)c(=O)c2ccccc12]

or a pharmaceutically acceptable salt thereof.

Non-limiting examples of NEMO stabilizing compounds of the present invention include:

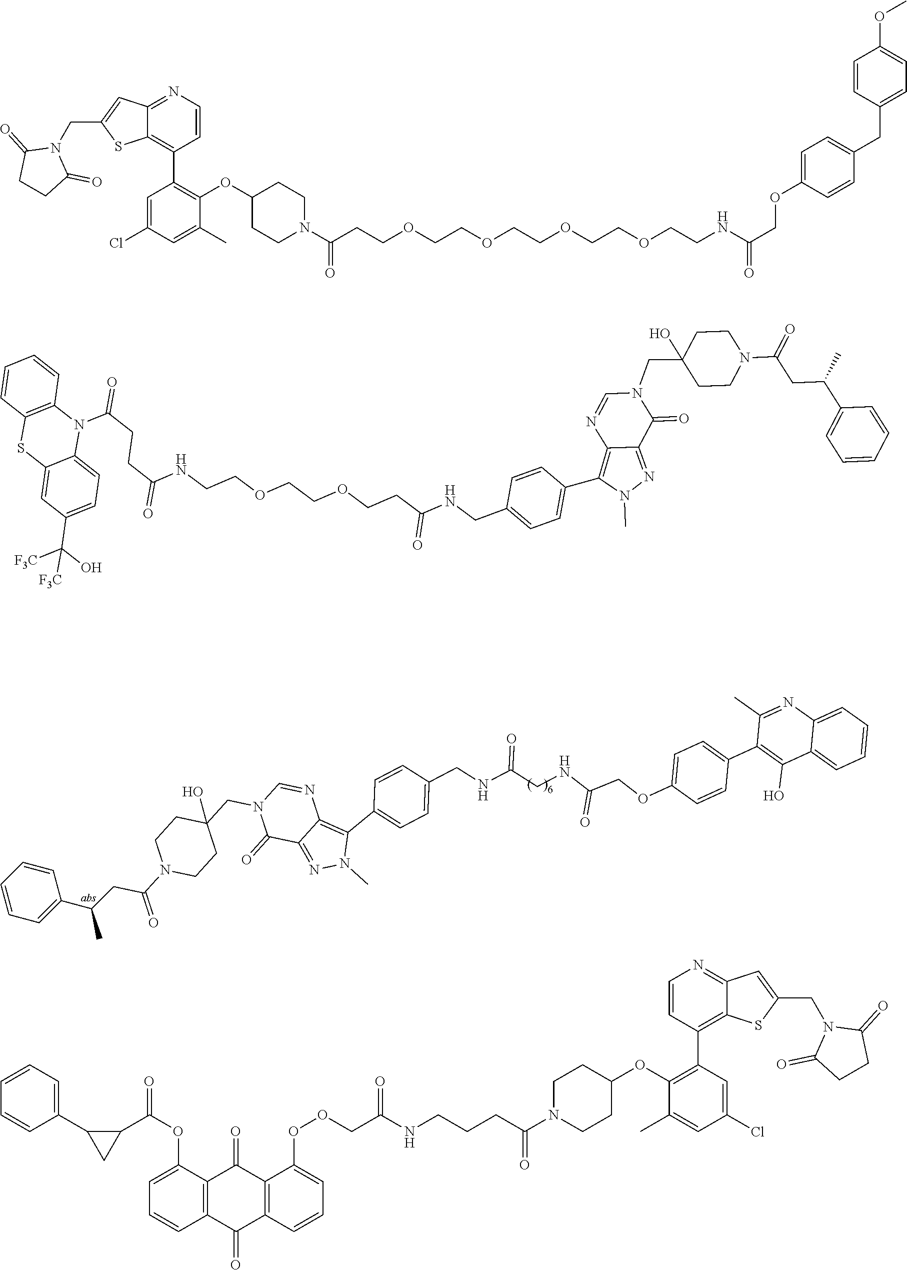

or a pharmaceutically acceptable salt thereof.

Non-limiting examples of AIP stabilizing compounds of the present invention include

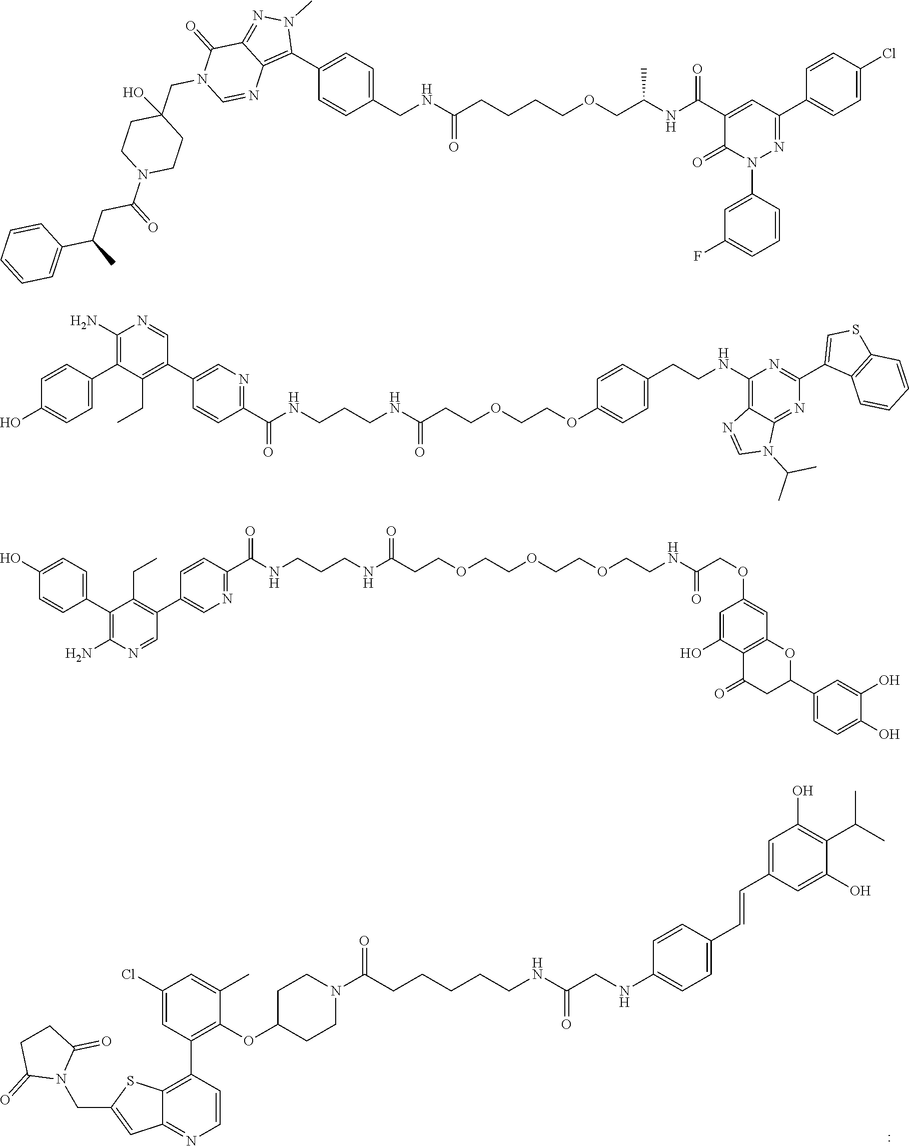 [SMILES: C[C@H](CC(=O)N1CCC(O)(Cn2cnc3c(-c4ccc(CNC(=O)CCCCOC[C@H](C)NC(=O)c5cc(-c6ccc(Cl)cc6)nn(-c6cccc(F)c6)c5=O)cc4)n(C)nc3c2=O)CC1)c1ccccc1 ; CCc1c(-c2ccc(C(=O)NCCCNC(=O)CCOCCOc3ccc(CCNc4nc(-c5csc6ccccc56)nc5c4ncn5C(C)C)cc3)nc2)cnc(N)c1-c1ccc(O)cc1 ; CCc1c(-c2ccc(C(=O)NCCCNC(=O)CCOCCOCCOCCNC(=O)COc3cc(O)c4c(c3)OC(c3ccc(O)c(O)c3)CC4=O)nc2)cnc(N)c1-c1ccc(O)cc1 ; Cc1cc(Cl)cc(-c2ccnc3cc(CN4C(=O)CCC4=O)sc23)c1OC1CCN(C(=O)CCCCCNC(=O)CNc2ccc(/C=C/c3cc(O)c(C(C)C)c(O)c3)cc2)CC1]

or a pharmaceutically acceptable salt thereof.

### Compounds of the Present Invention

In certain embodiments the compound of the present invention is selected from.

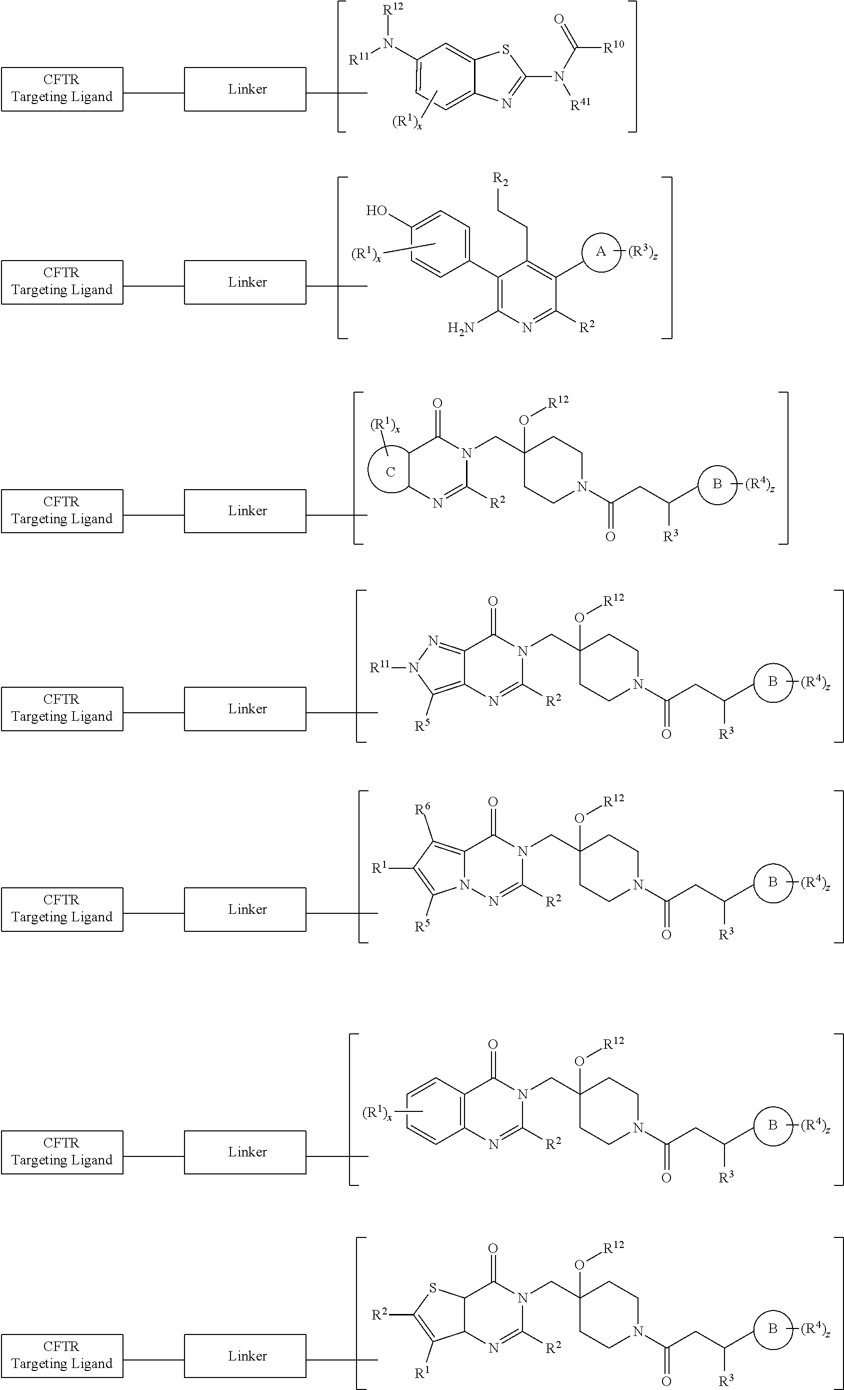

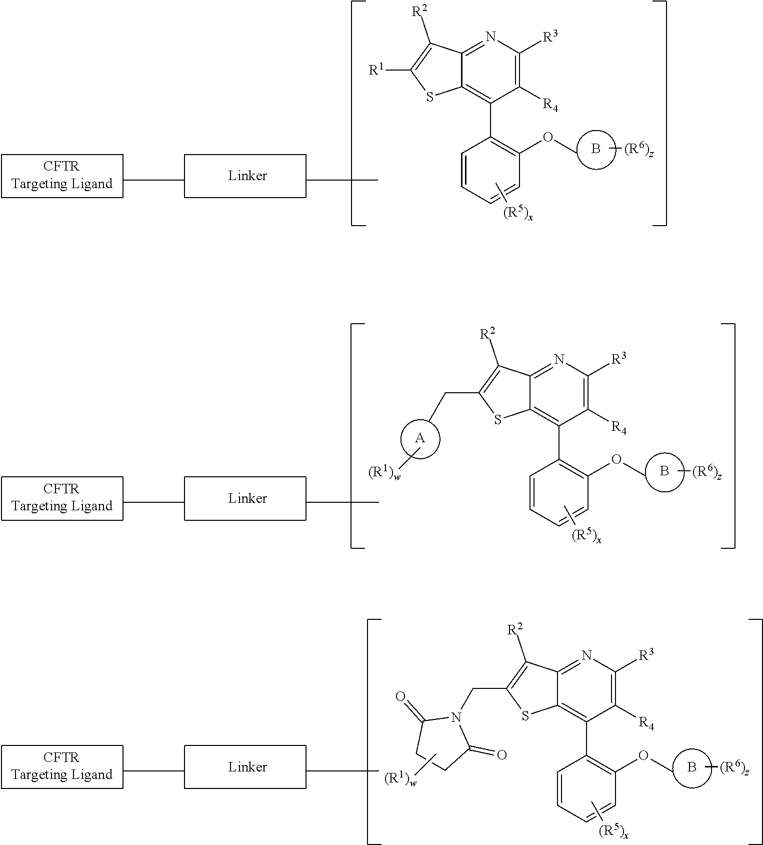

or a pharmaceutically acceptable salt thereof.

In certain embodiments the compound of the present invention is selected from:

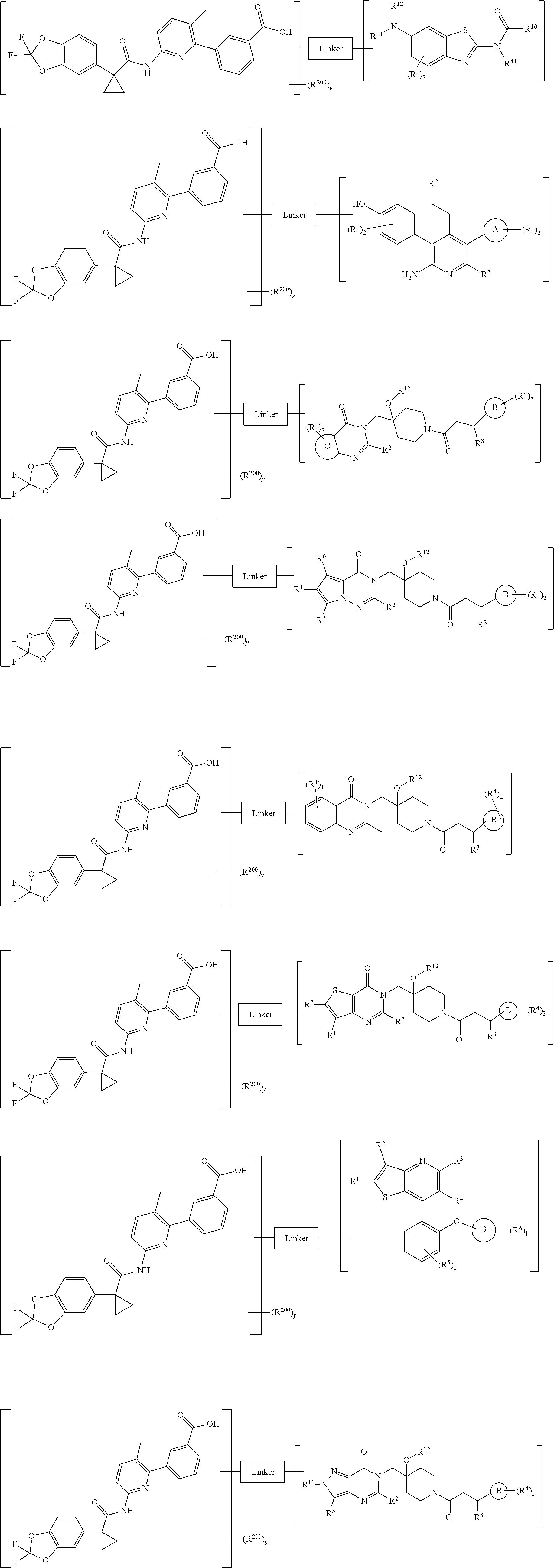

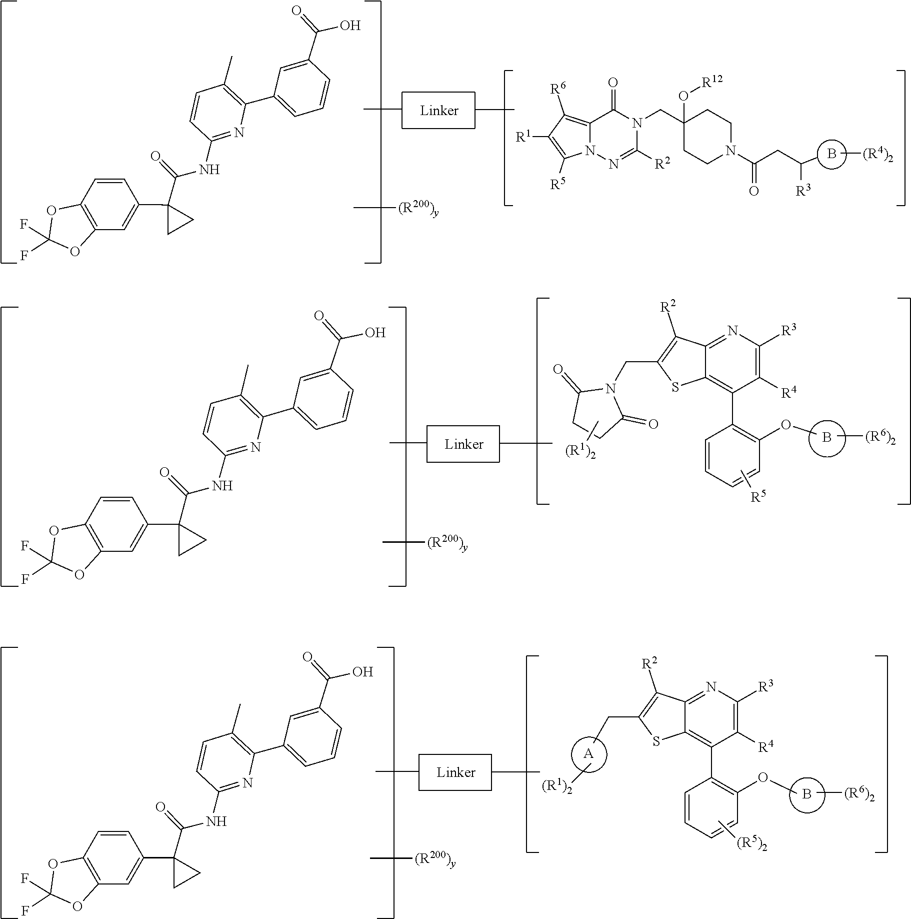

or a pharmaceutically acceptable salt thereof.

In certain embodiments the BAX stabilizing compound of the present invention is selected from:

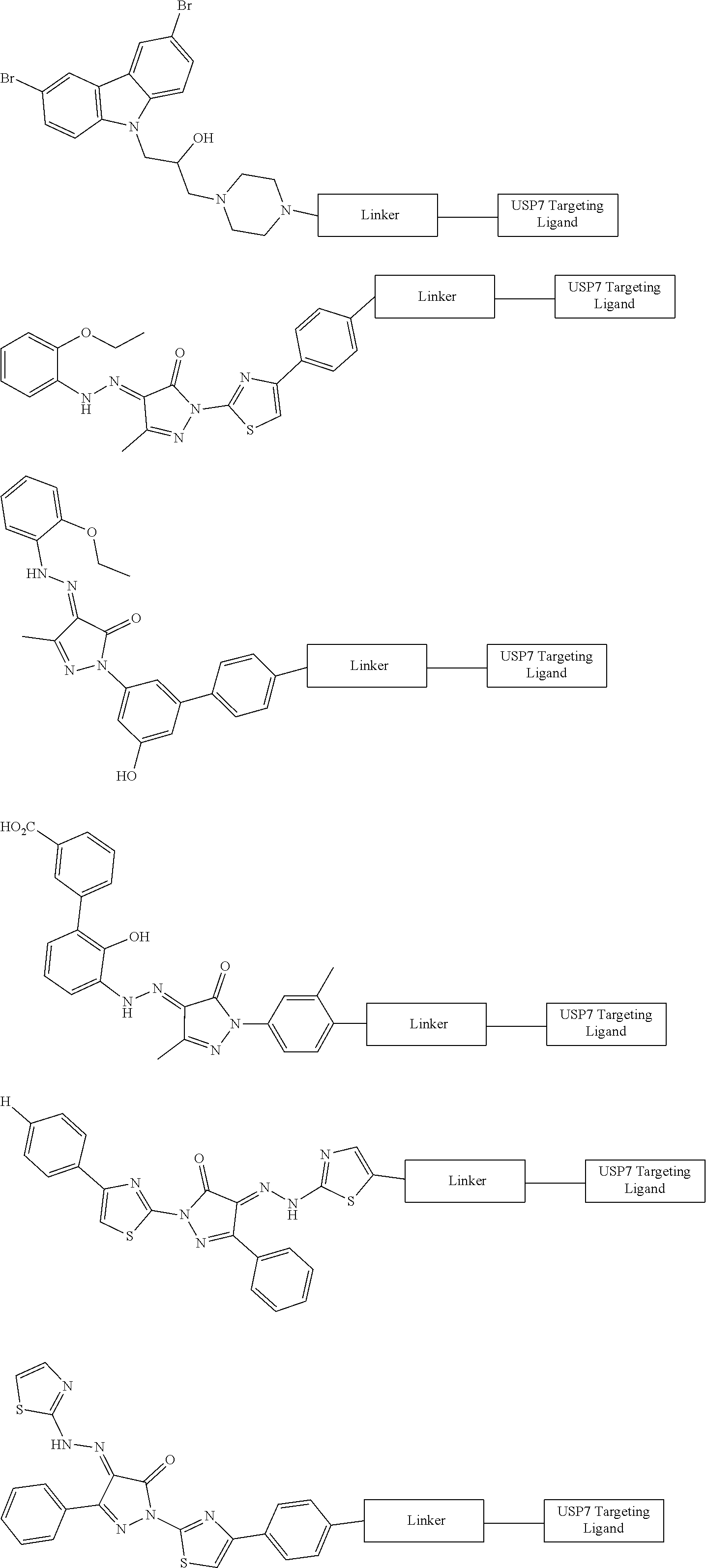 [SMILES: CN1CCN(CC(O)Cn2c3ccc(Br)cc3c3cc(Br)ccc32)CC1 ; CCOc1ccccc1N/N=C1/C(=O)N(c2nc(-c3ccc(C)cc3)cs2)N=C1C ; CCOc1ccccc1N/N=C1/C(=O)N(c2cc(O)cc(-c3ccc(C)cc3)c2)N=C1C ; CC1=NN(c2ccc(C)c(C)c2)C(=O)/C1=N/Nc1cccc(-c2cccc(C(=O)O)c2)c1O ; Cc1cnc(N/N=C2/C(=O)N(c3nc(-c4ccccc4)cs3)N=C2c2ccccc2)s1 ; Cc1ccc(-c2csc(N3N=C(c4ccccc4)/C(=N\Nc4nccs4)C3=O)n2)cc1]

or a pharmaceutically acceptable salt thereof.

In certain embodiments the PKLR stabilizing compound of the present invention is selected from:

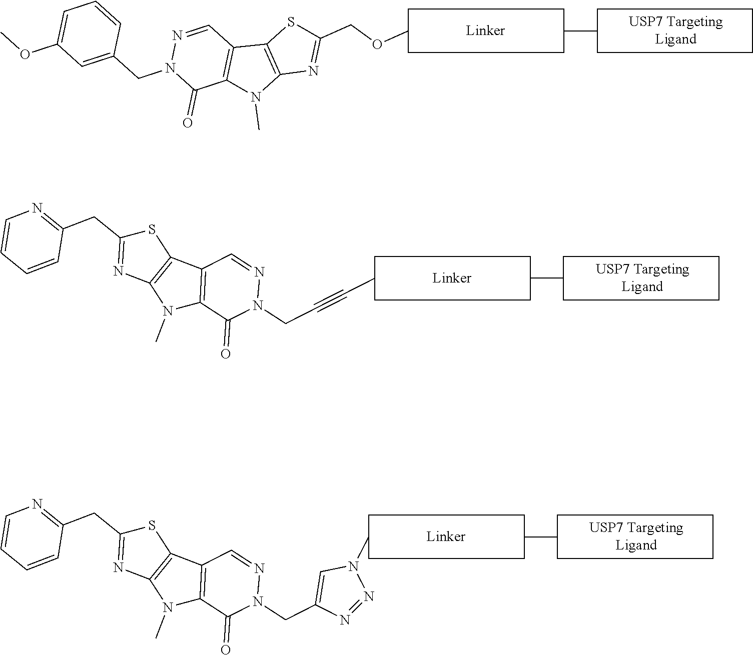 [SMILES: COc1cccc(Cn2ncc3c4sc(COC5CCC(C6CCCCC6)CC5)nc4n(C)c3c2=O)c1 ; Cn1c2nc(Cc3ccccn3)sc2c2cnn(CC#CC3CCC(C4CCCCC4)CC3)c(=O)c21 ; Cn1c2nc(Cc3ccccn3)sc2c2cnn(Cc3cn(C4CCC(C5CCCCC5)CC4)nn3)c(=O)c21]

or a pharmaceutically acceptable salt thereof.

In certain embodiments the KEAP1 stabilizing compound of the present invention is selected from:

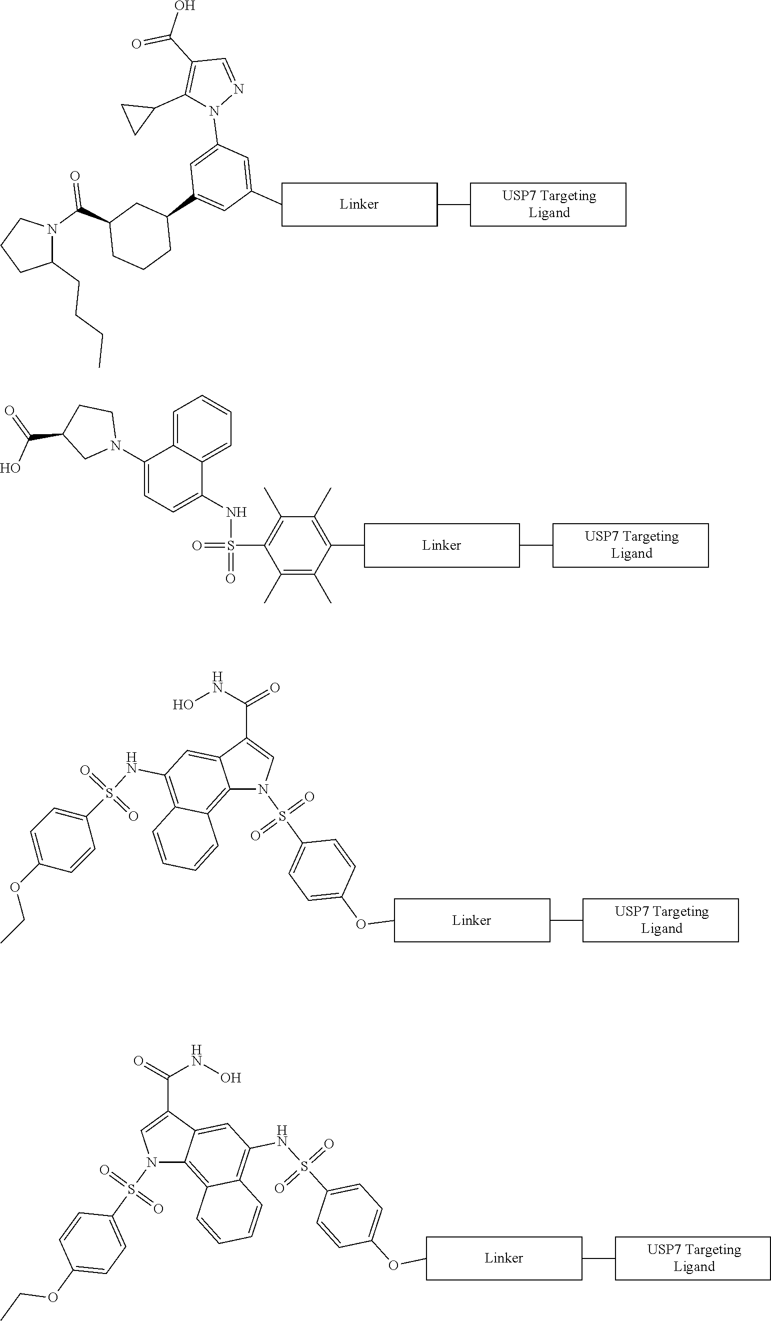

or a pharmaceutically acceptable salt thereof.

In certain embodiments the IRAK4 stabilizing compound of the present invention is selected from:

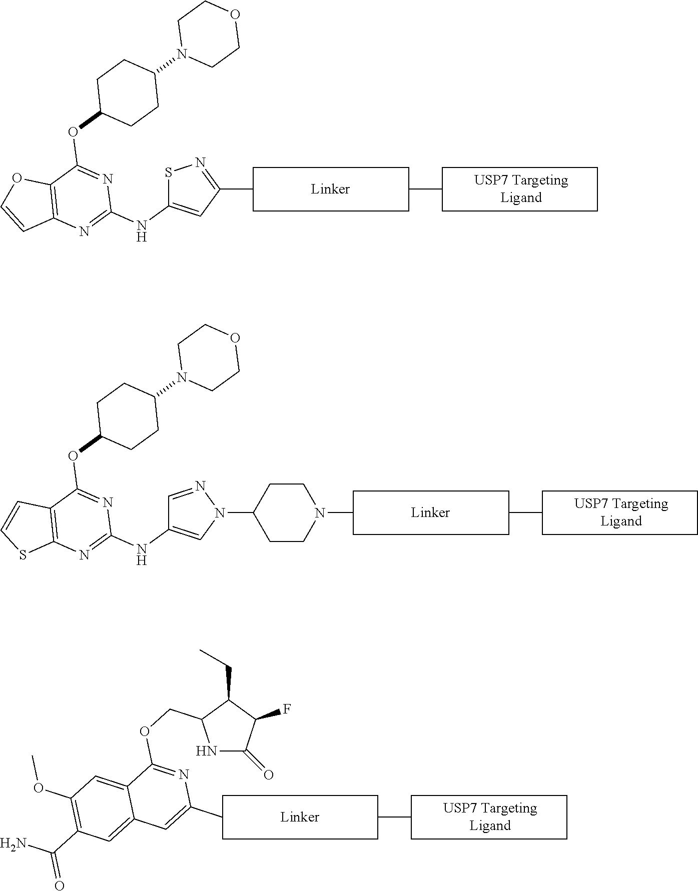

or a pharmaceutically acceptable salt thereof.

In certain embodiments the PTEN stabilizing compound of the present invention is selected from:

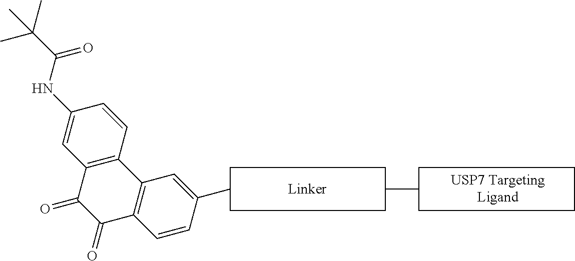 [SMILES: CC(C)(C)C(=O)Nc1ccc2c(c1)C(=O)C(=O)c1ccc(C3CCC(C4CCCCC4)CC3)cc1-2]

or a pharmaceutically acceptable salt thereof.

In certain embodiments the TK2 stabilizing compound of the present invention is selected from:

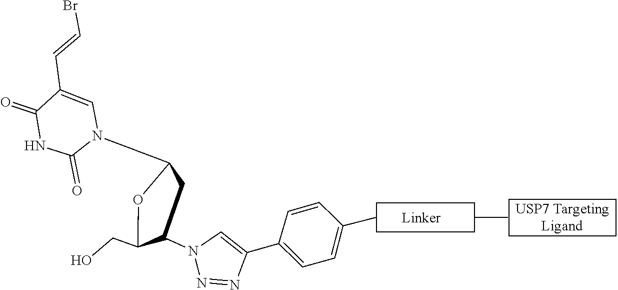

or a pharmaceutically acceptable salt thereof.

In certain embodiments the KCNQ1 stabilizing compound of the present invention is selected from:

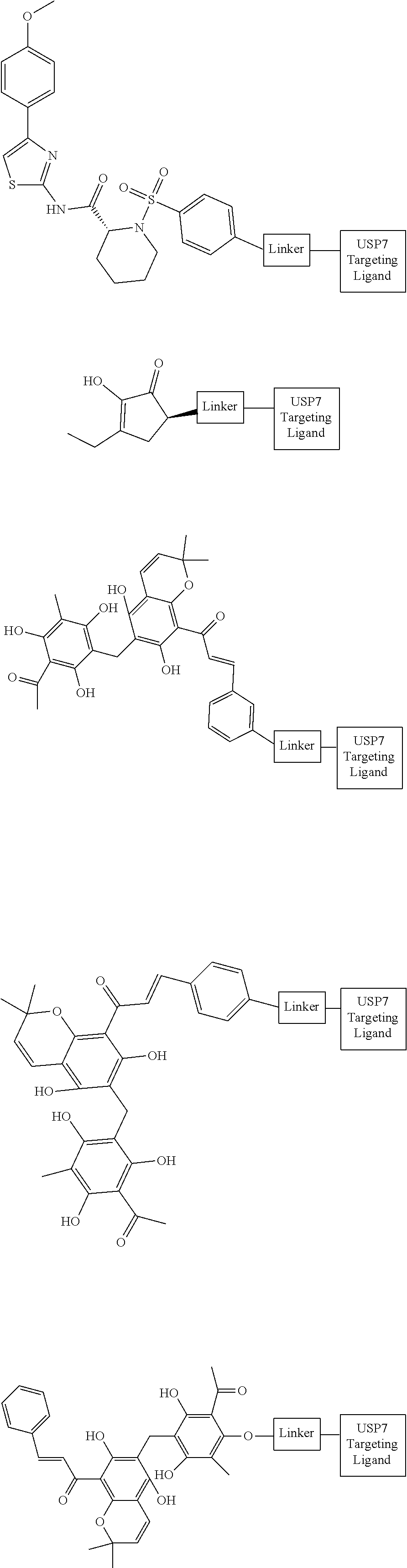

or a pharmaceutically acceptable salt thereof.

In certain embodiments the compound of the present invention is selected from:

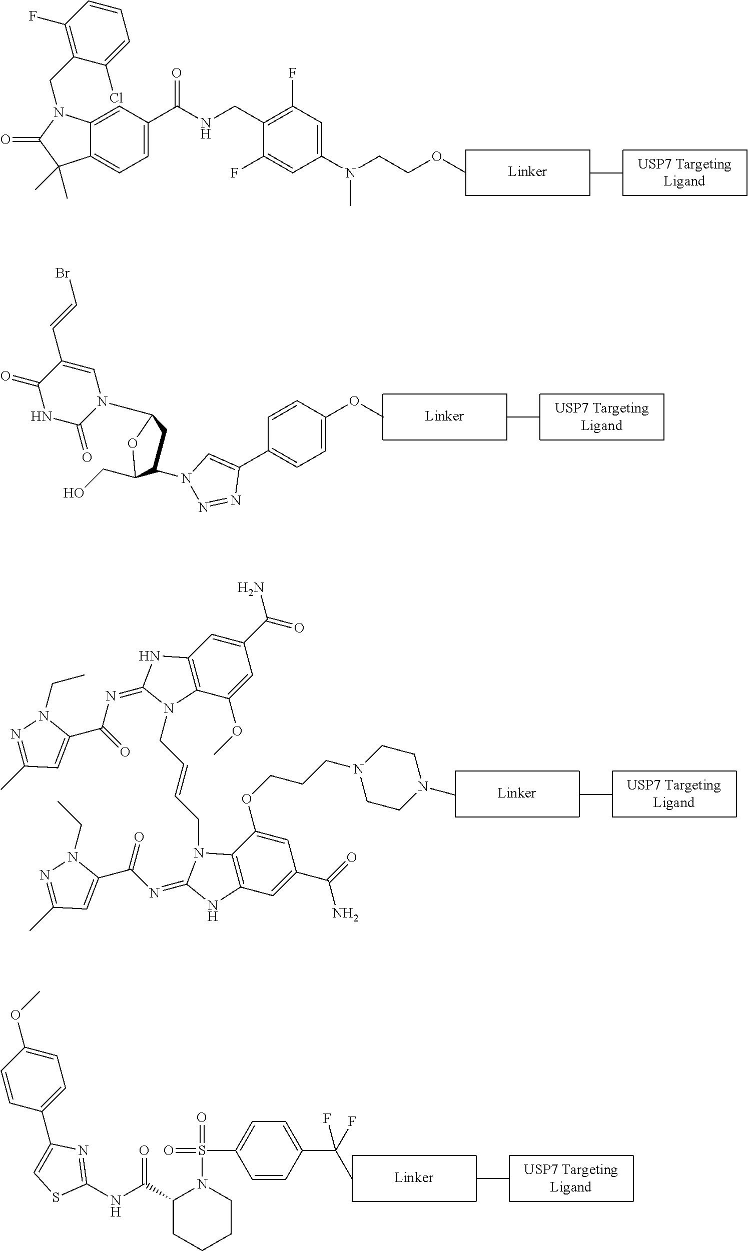

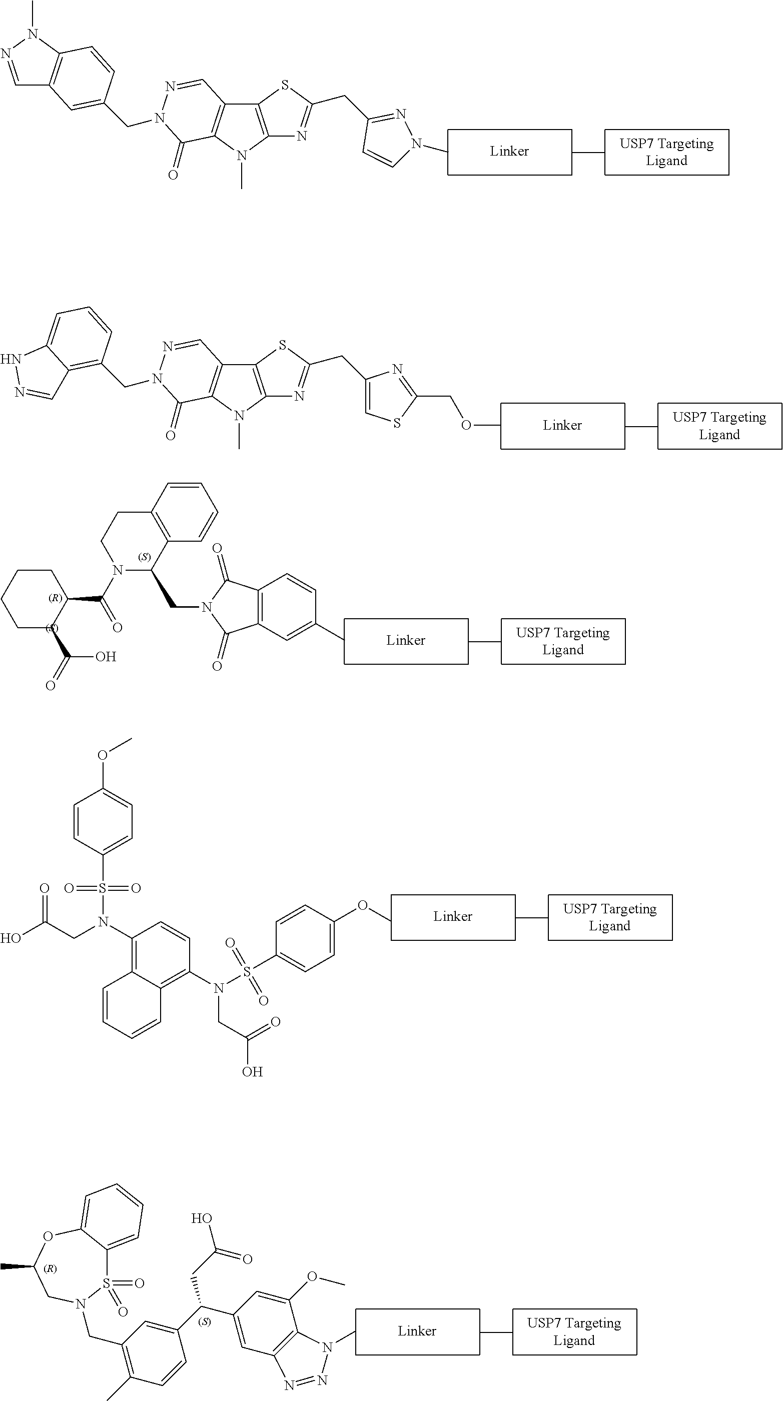

or a pharmaceutically acceptable salt thereof.

### Additional Embodiments of the Present Invention

1. A compound of Formula

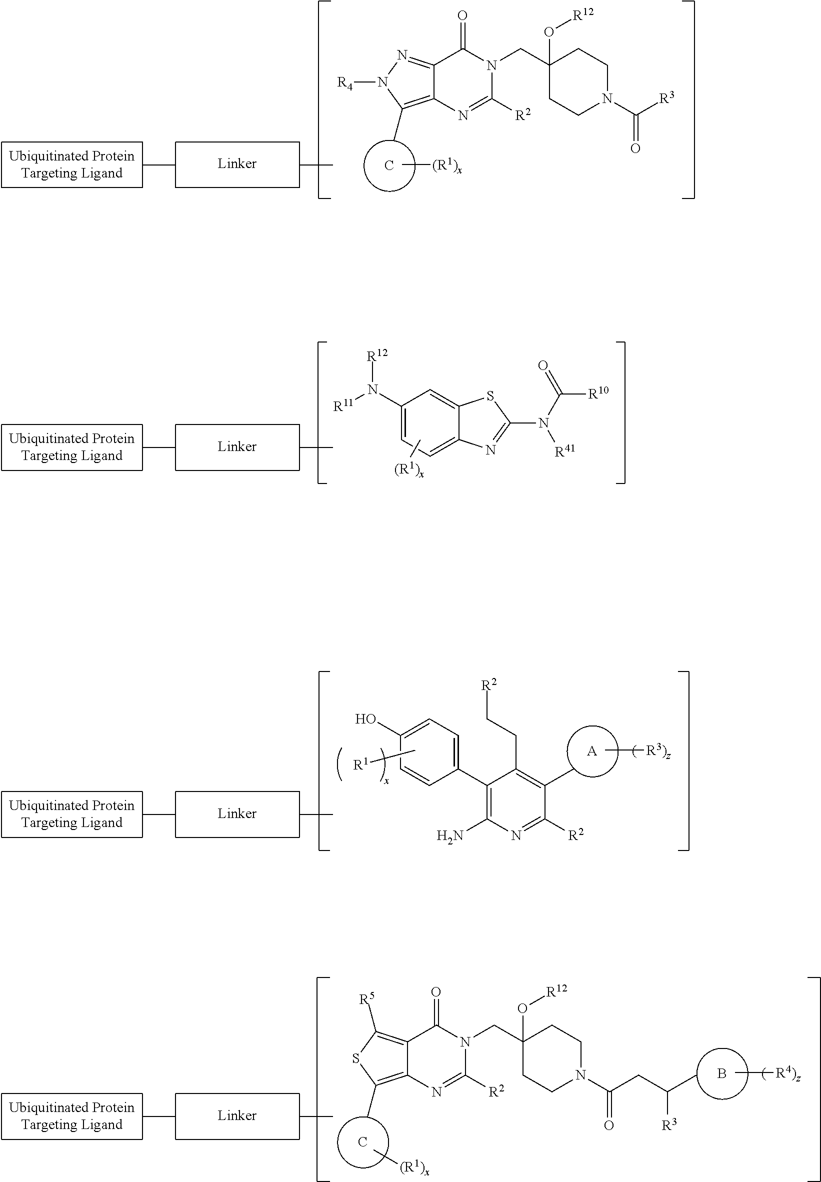

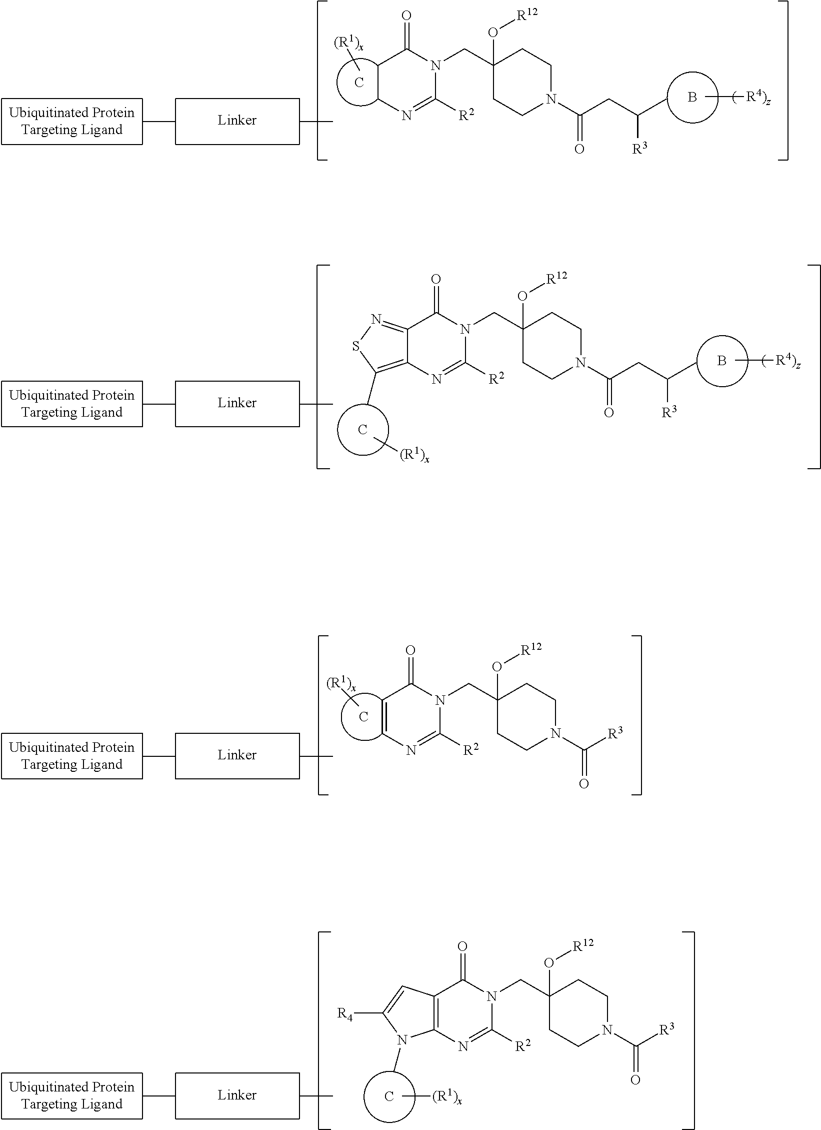

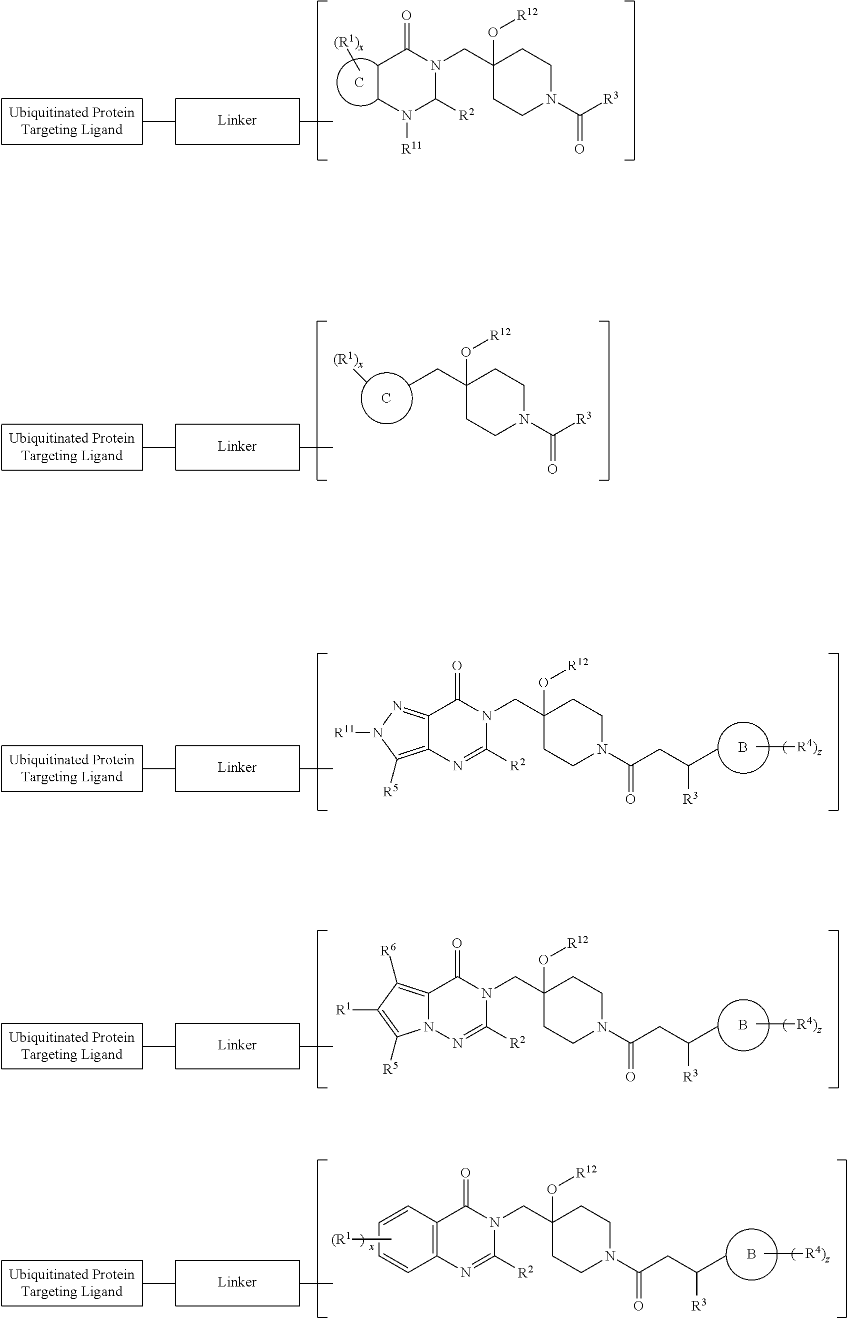

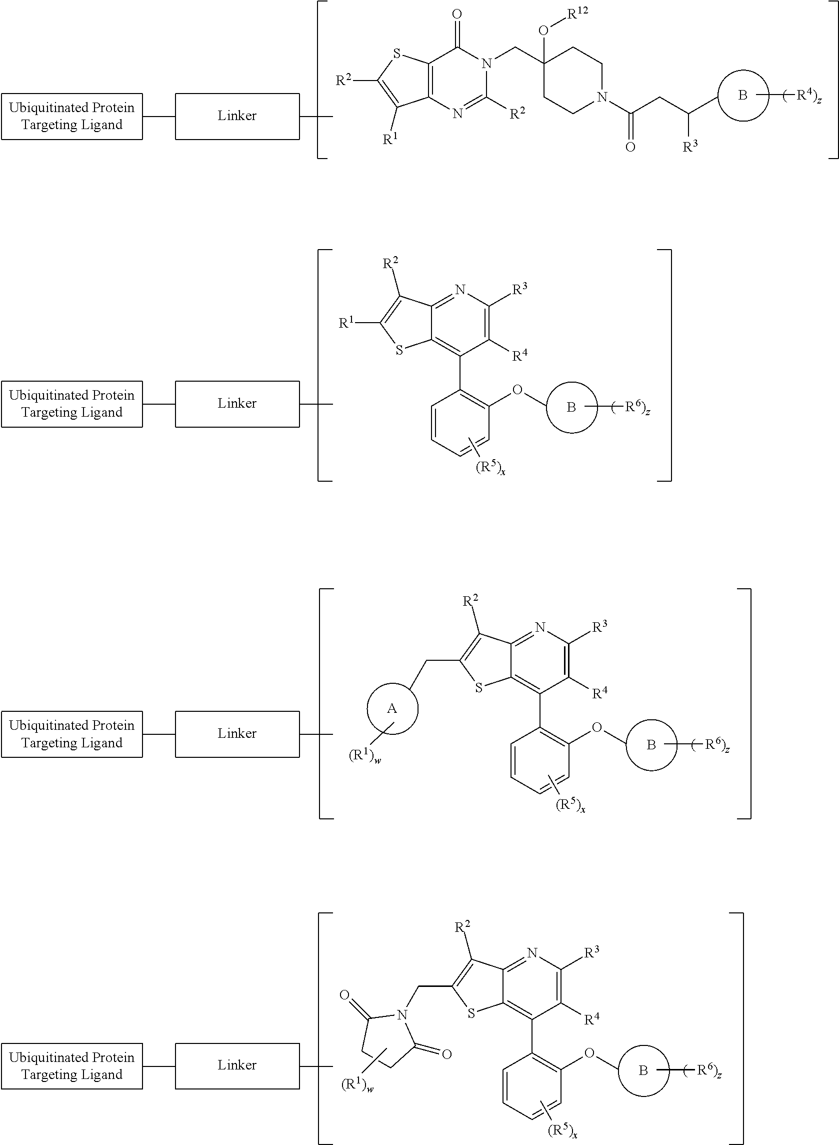

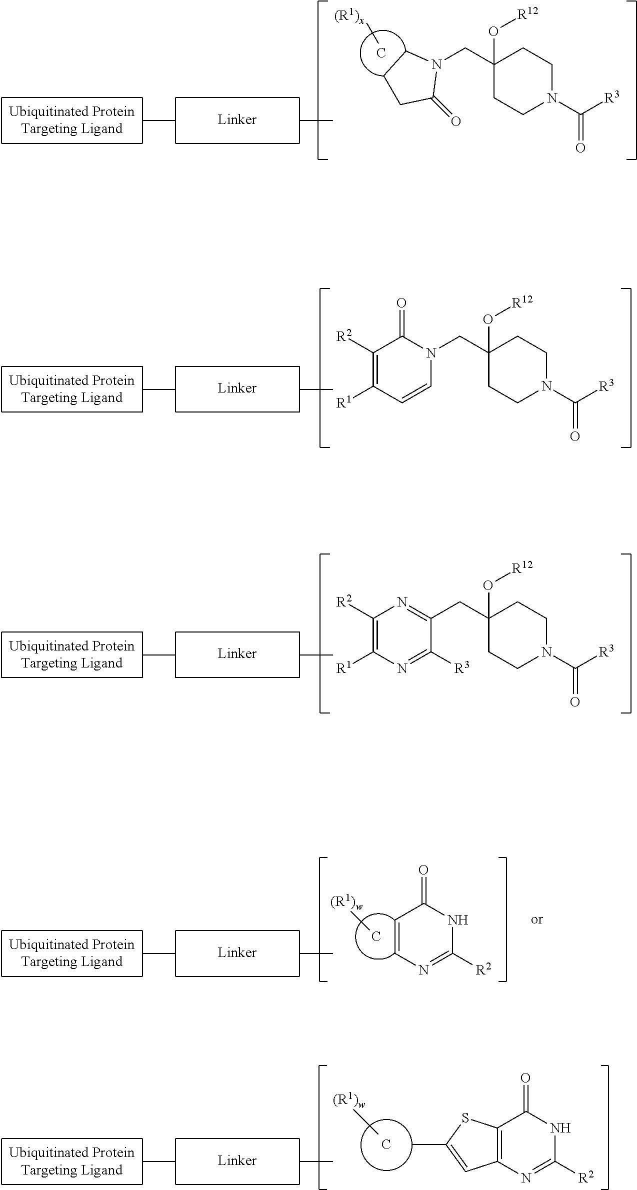

or a pharmaceutically acceptable salt thereof;
wherein:

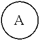

is an aryl, heteroaryl, heterocycle, or cycloalkyl group;

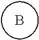

is an aryl, heteroaryl, heterocycle, or cycloalkyl group;

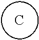

is an aryl, heteroaryl, heterocycle, or cycloalkyl group;

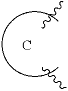

is a fused heterocycle, aryl, heteroaryl, cycloalkyl, or cycloalkenyl group;
x is 0, 1, 2, 3, or 4 as allowed by valence;
z is 0, 1, 2, 3, or 4 as allowed by valence;
w is 0, 1, 2, 3, or 4 as allowed by valence;
R¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹¹, wherein each alkyl, haloalkyl, alkenyl, alkynyl, heterocycle, aryl, and heteroaryl is optionally substituted as allowed by valence with 1, 2, 3, or 4 substituents selected from R²¹;
R² is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²²;
R³ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²³;
R⁴ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁴;
R⁵ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁵;
R⁶ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁶;
R¹⁰ is independently selected at each instance from hydrogen, alkyl, haloalkyl, alkenyl, alkynyl, —OR¹¹, —NR¹¹R¹², —SR¹¹, aryl, heterocycle, and heteroaryl; each of which alkyl, haloalkyl, alkenyl, alkynyl, heterocycle, aryl, and heteroaryl is optionally substituted as allowed by valence with 1, 2, 3, or 4 substituents selected from R³⁰;
R¹¹ and R¹² are independently selected at each instance from hydrogen, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
R²¹, R²², R²³, R²⁴, R²⁵, and R²⁶ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R³⁰ and R³¹ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R⁴⁰ is independently selected at each instance from hydrogen, alkyl, haloalkyl, alkenyl, alkynyl, aryl, heterocycle, heteroaryl, amino, hydroxyl, alkoxy, —NHalkyl, and —N(alkyl)₂, each of which except hydrogen is optionally substituted as allowed by valence with 1, 2, 3, or 4 substituents selected from R⁴³;
R⁴¹ and R⁴² are independently selected at each instance from hydrogen, alkyl, haloalkyl, alkenyl, alkynyl, aryl, heterocycle, and heteroaryl; each of which except hydrogen is optionally substituted as allowed by valence with 1, 2, 3, or 4 substituents selected from R⁴³;
R⁴³ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl;
the Ubiquitinated Protein Targeting Ligand is a ligand that binds a Target Ubiquitinated Protein;
the Linker is a bond or a bivalent moiety that links the Protein Targeting Ligand and the USP7 Targeting; and
and wherein Linker-Ubiquitinated Protein Targeting Ligand replaces a R¹, R², R³, R⁴, R⁵, R⁶, R¹⁰, R¹¹, or R¹² group; or Linker-Ubiquitinated Protein Targeting Ligand is covalently attached to a R¹, R², R³, R⁴, R⁵, R⁶, R¹⁰, R¹¹, or R¹² group as allowed by valence; or Linker-Ubiquitinated Protein Targeting Ligand is covalently attached in a position other than R¹, R², R³, R⁴, R⁵, R⁶, R¹⁰, R¹¹, and R¹².
2. The compound of embodiment 1, wherein Linker-Ubiquitinated Protein Targeting Ligand replaces a R¹, R², R³, R⁴, R⁵, R⁶, R¹⁰, R¹¹, or R2 group.
3. The compound of embodiment 1, wherein Linker-Ubiquitinated Protein Targeting Ligand is covalently attached to a R¹, R², R³, R⁴, R⁵, R⁶, R¹⁰, R¹¹, or R² group as allowed by valence.
4. The compound of embodiment 1, wherein Linker-Ubiquitinated Protein Targeting Ligand is covalently attached in a position other than R¹, R², R³, R⁴, R⁵, R⁶, R¹⁰, R¹¹, and R¹².
5. The compound of embodiment 1, wherein Linker-Ubiquitinated Protein Targeting Ligand replaces R¹.
6. The compound of embodiment 1, wherein Linker-Ubiquitinated Protein Targeting Ligand replaces R².
7. The compound of embodiment 1, wherein Linker-Ubiquitinated Protein Targeting Ligand replaces R³.
8. The compound of embodiment 1, wherein Linker-Ubiquitinated Protein Targeting Ligand replaces R⁴.
9. The compound of embodiment 1, wherein Linker-Ubiquitinated Protein Targeting Ligand replaces R⁵.
10. The compound of embodiment 1, wherein Linker-Ubiquitinated Protein Targeting Ligand replaces R⁶.
11. The compound of embodiment 1, wherein Linker-Ubiquitinated Protein Targeting Ligand replaces R¹⁰.
12. The compound of embodiment 1, wherein Linker-Ubiquitinated Protein Targeting Ligand replaces R¹¹.
13. The compound of embodiment 1, wherein Linker-Ubiquitinated Protein Targeting Ligand replaces R¹².
14. The compound of embodiment 1, wherein the compound is of Formula:

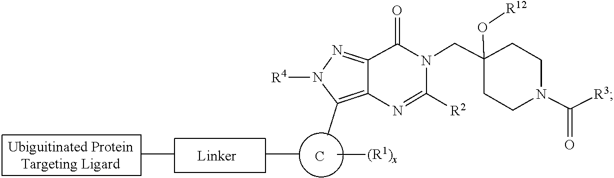

or a pharmaceutically acceptable salt thereof.
15. The compound of embodiment 1, wherein the compound is of Formula:

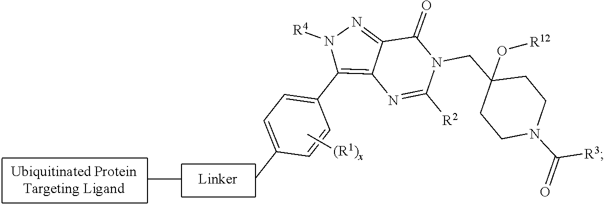

or a pharmaceutically acceptable salt thereof.
16. The compound of embodiment 1, wherein the compound is of Formula:

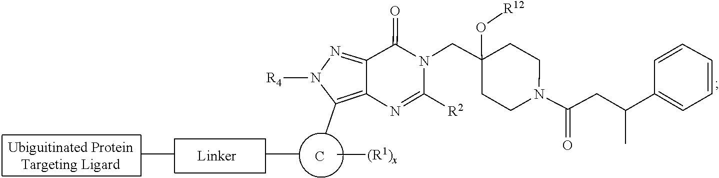

or a pharmaceutically acceptable salt thereof.
17. The compound of any one of embodiments 1-16, wherein R⁴ is methyl.
18. The compound of any one of embodiments 1-16, wherein R⁴ is hydrogen.
19. The compound of any one of embodiments 1-18, wherein R² is hydrogen.
20. The compound of any one of embodiments 1-18, wherein R² is alkyl, haloalkyl, or halogen.
21. The compound of embodiment 1, wherein the compound is of Formula:

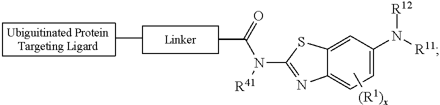

or a pharmaceutically acceptable salt thereof.
22. The compound of embodiment 1, wherein the compound is of Formula:

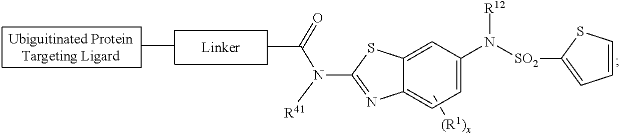

or a pharmaceutically acceptable salt thereof.
23. The compound of embodiment 21 or 22, wherein R⁴¹ is hydrogen.
24. The compound of embodiment 21 or 22, wherein R⁴¹ is alkyl.
25. The compound of any one of embodiments 1-24, wherein R¹² is hydrogen.
26. The compound of any one of embodiments 1-24, wherein R¹² is alkyl.
27. The compound of any one of embodiments 1-26, wherein x is 0.
28. The compound of any one of embodiments 1-26, wherein x is 1.
29. The compound of any one of embodiments 1-26, wherein x is 2.
30. The compound of any one of embodiments 1-26, wherein x is 3.
31. The compound of any one of embodiments 28-30, wherein R¹ is selected from F, Cl, alkyl, and haloalkyl.
32. The compound of any one of embodiments 1-31, wherein Linker is

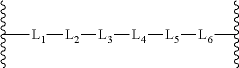

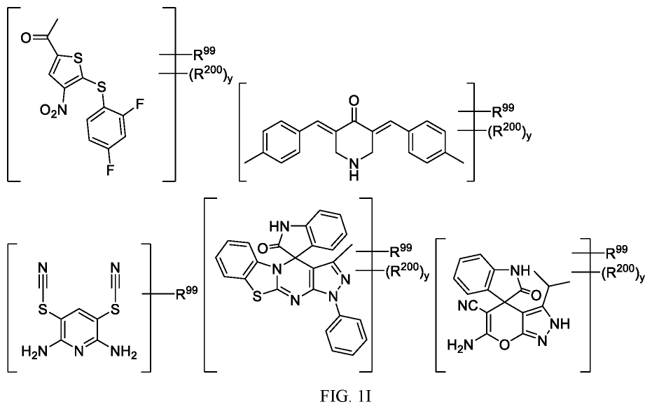

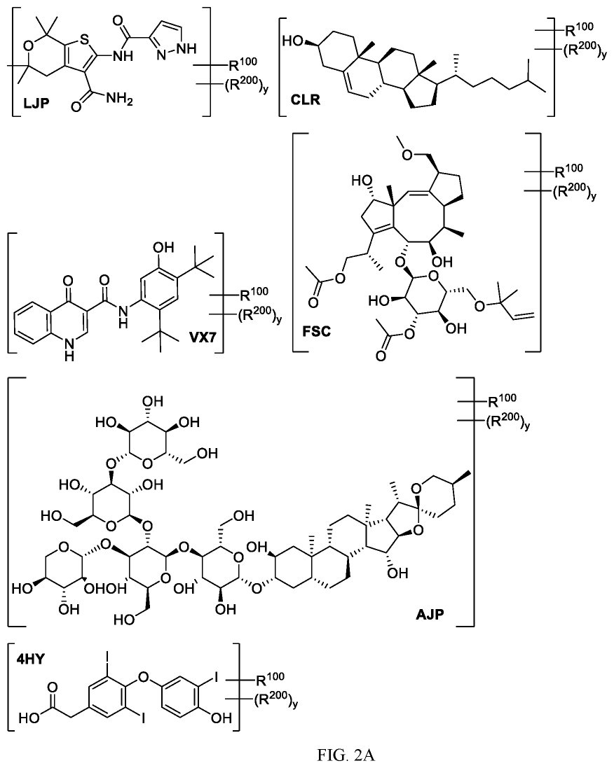

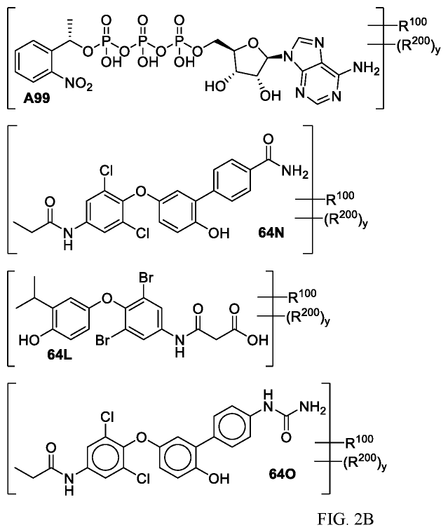

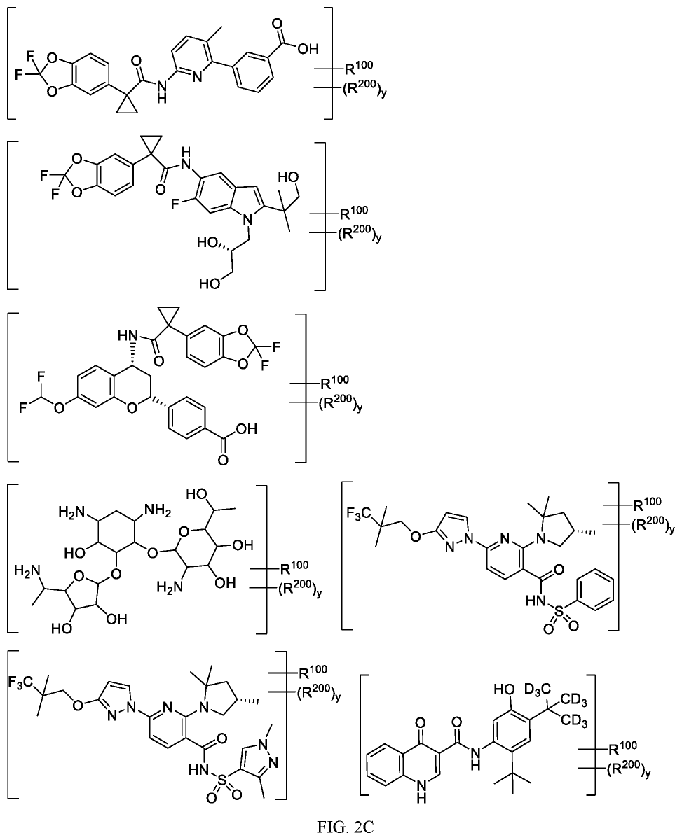

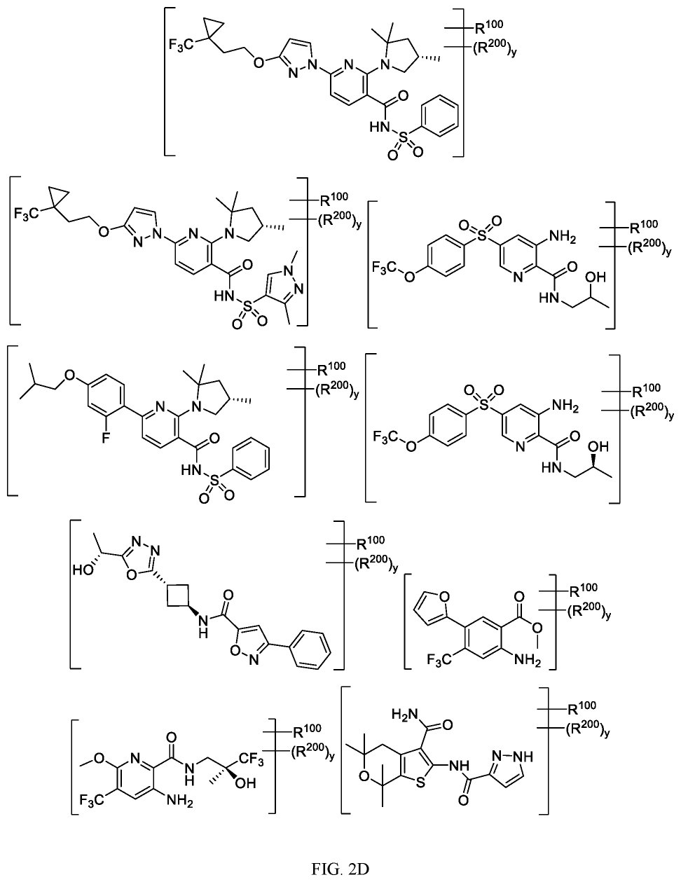

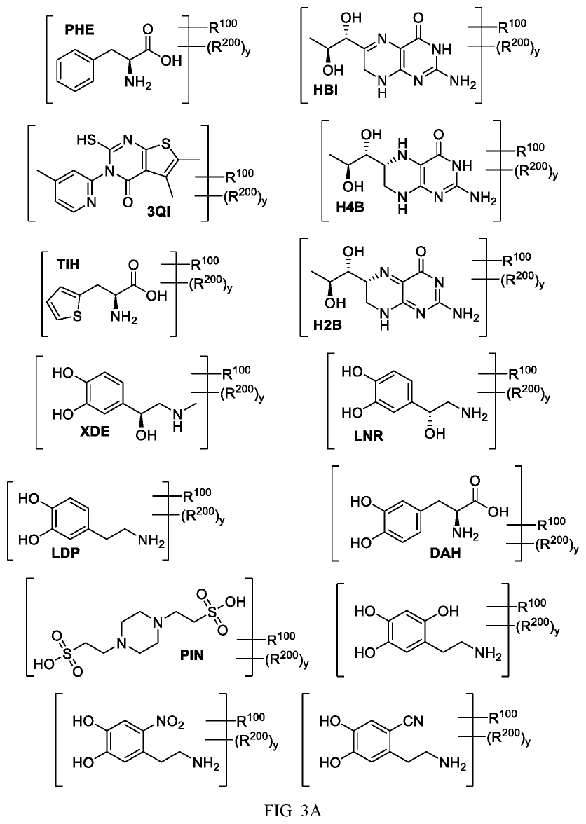

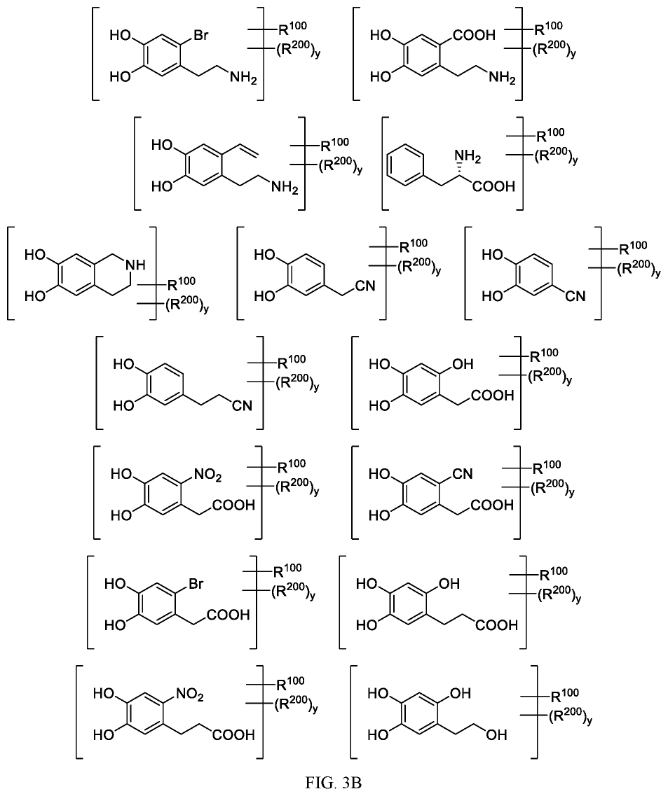

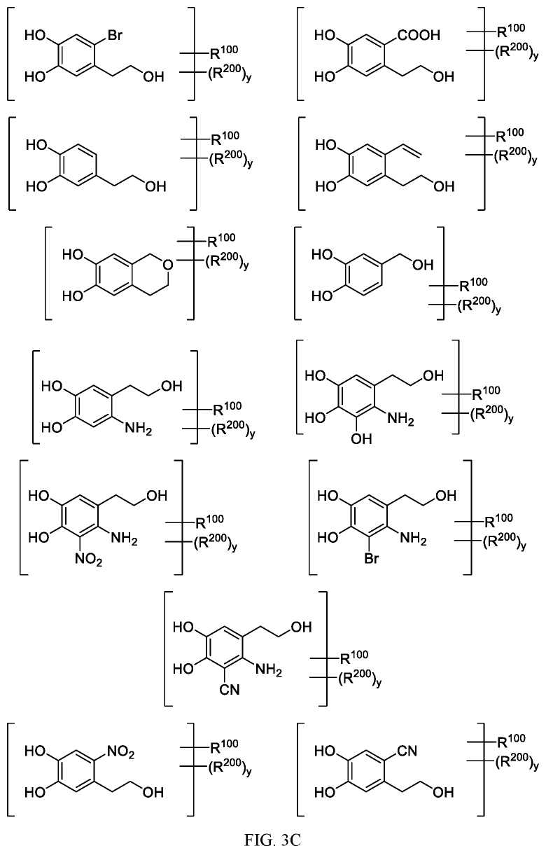

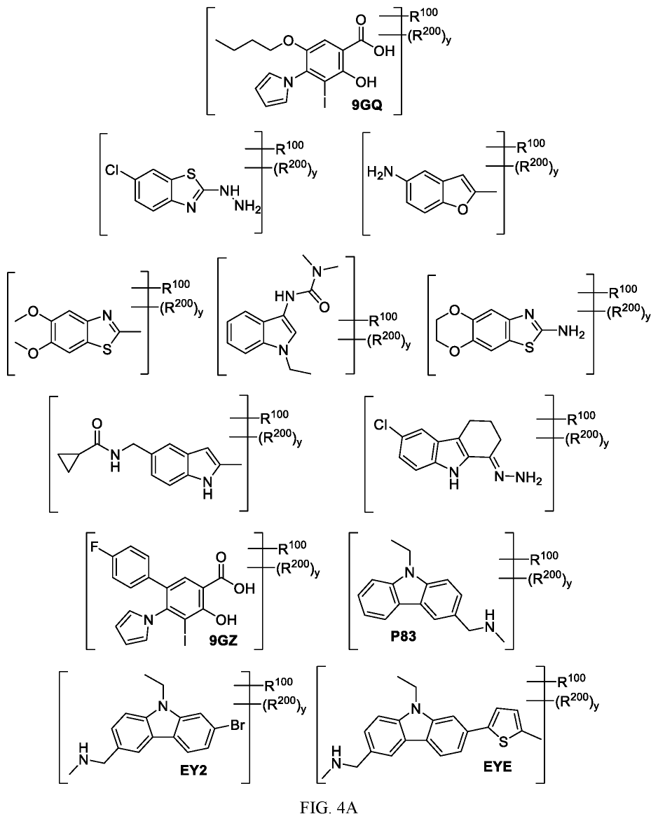

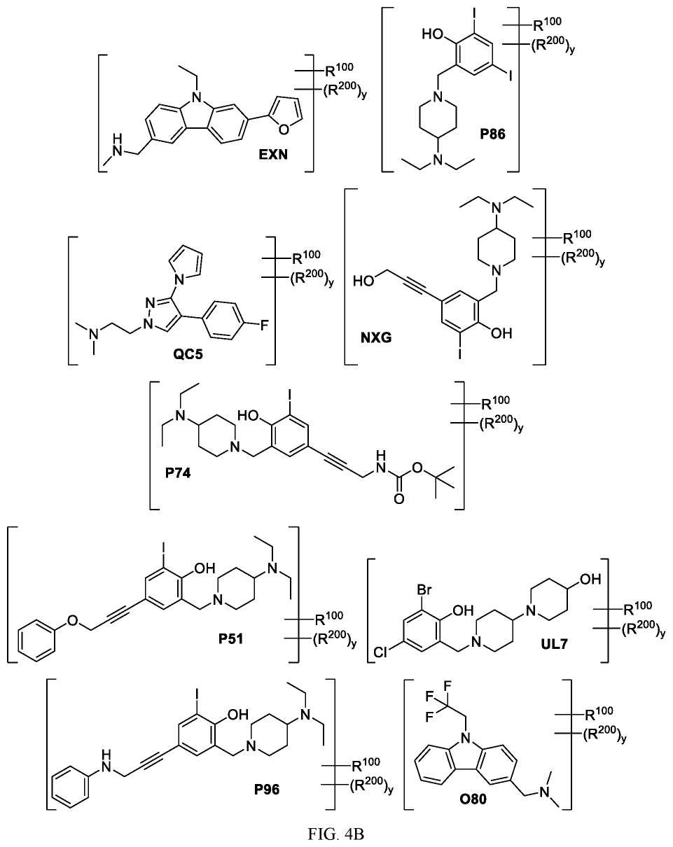

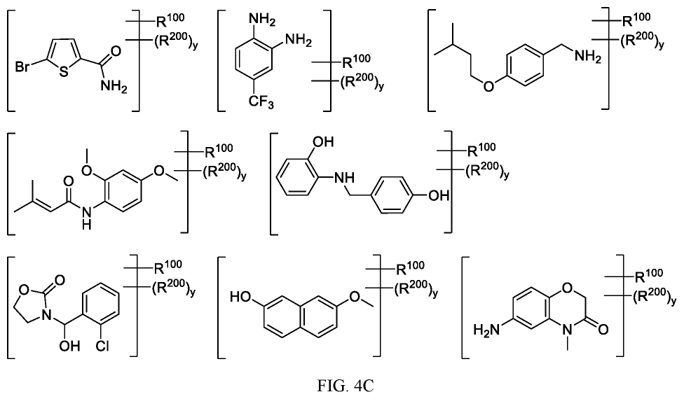

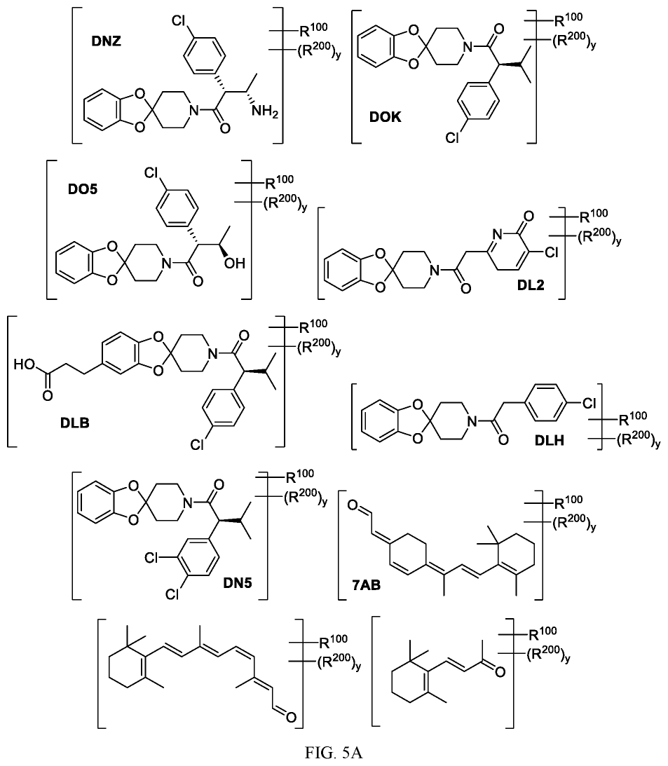

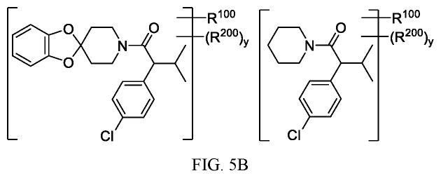

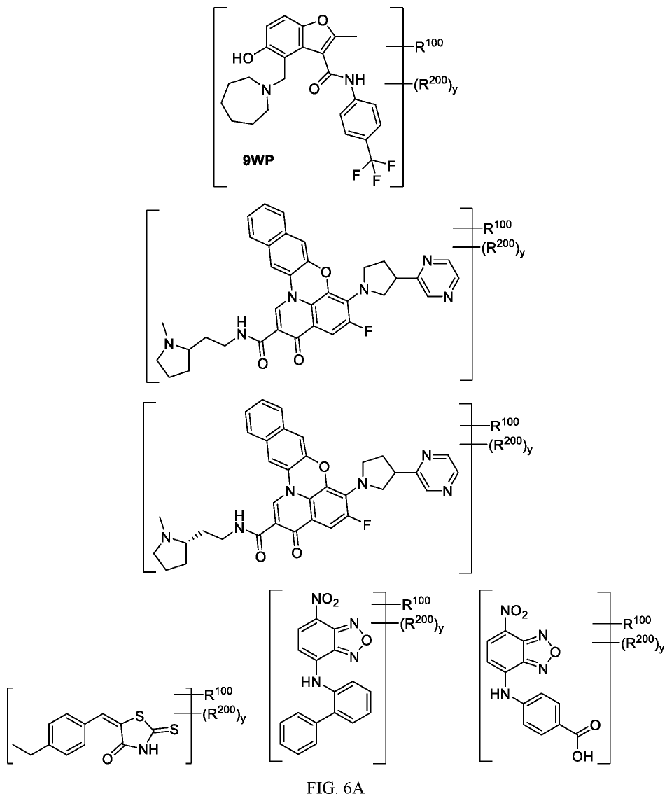

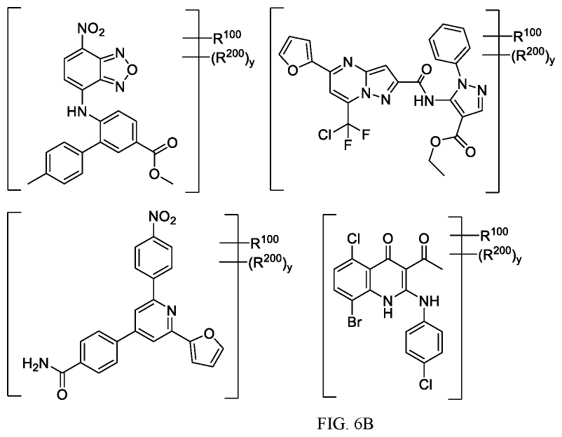

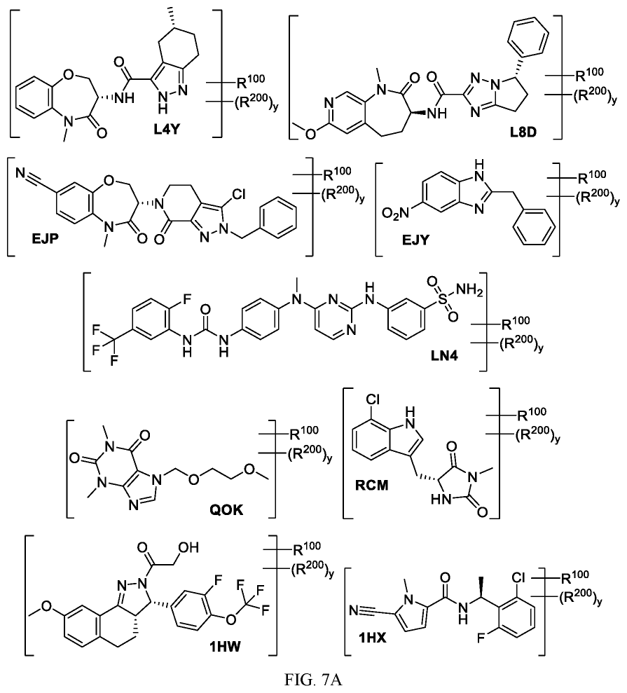

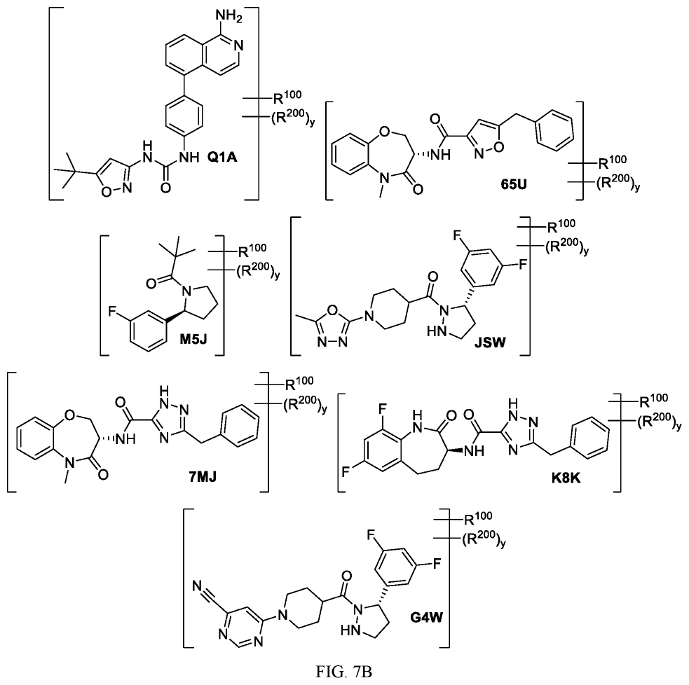

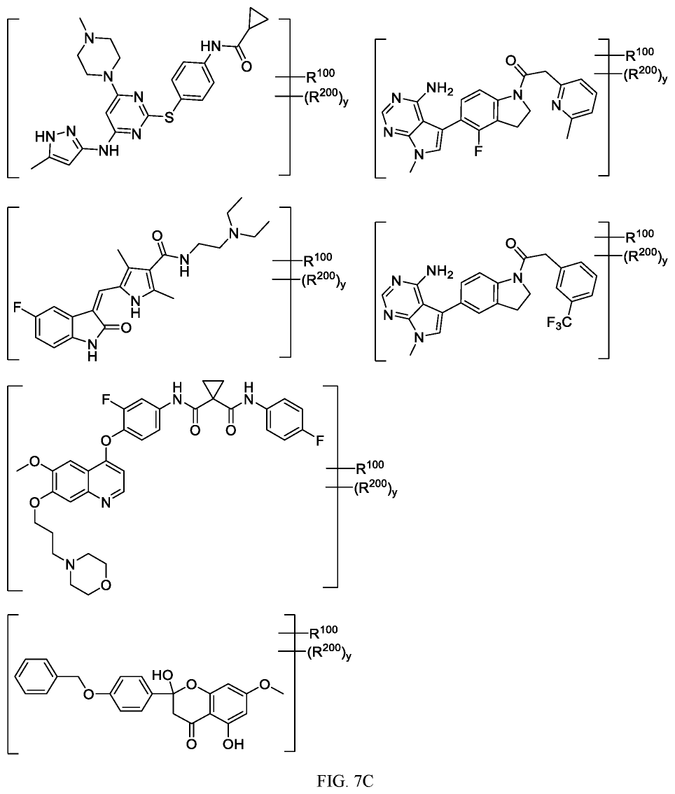

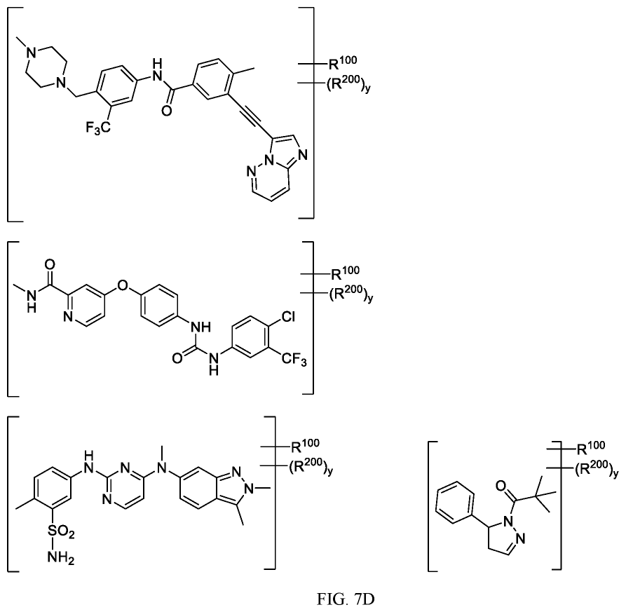

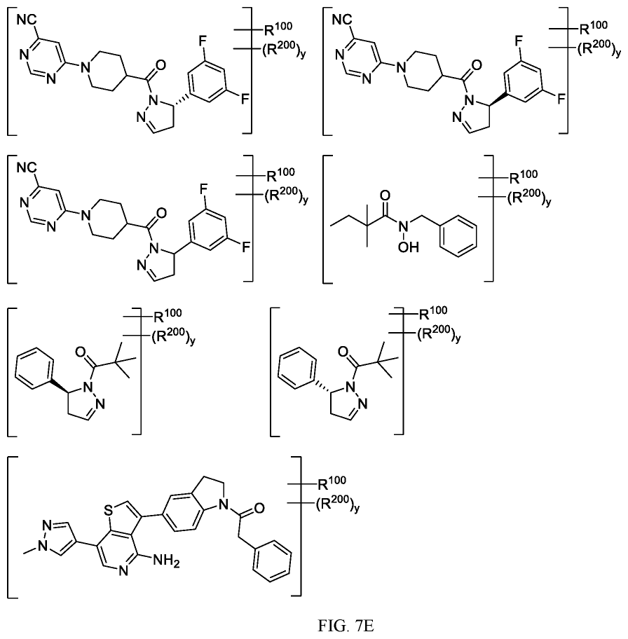

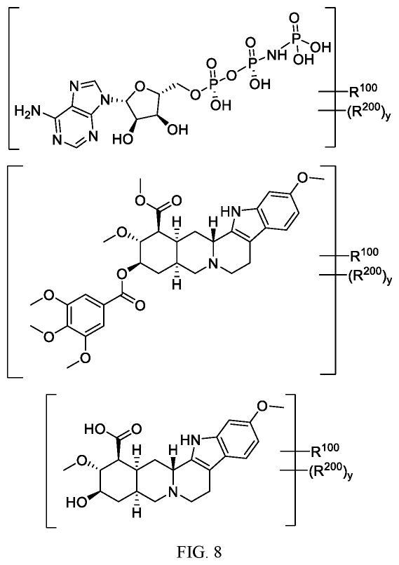

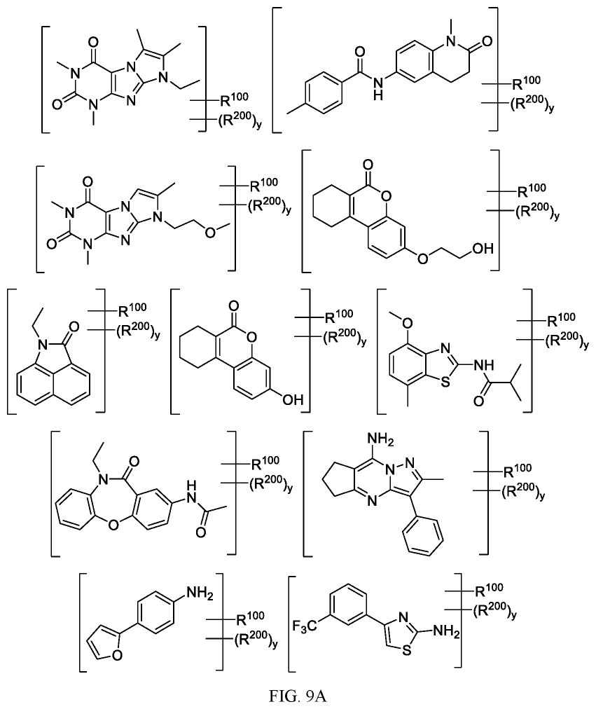

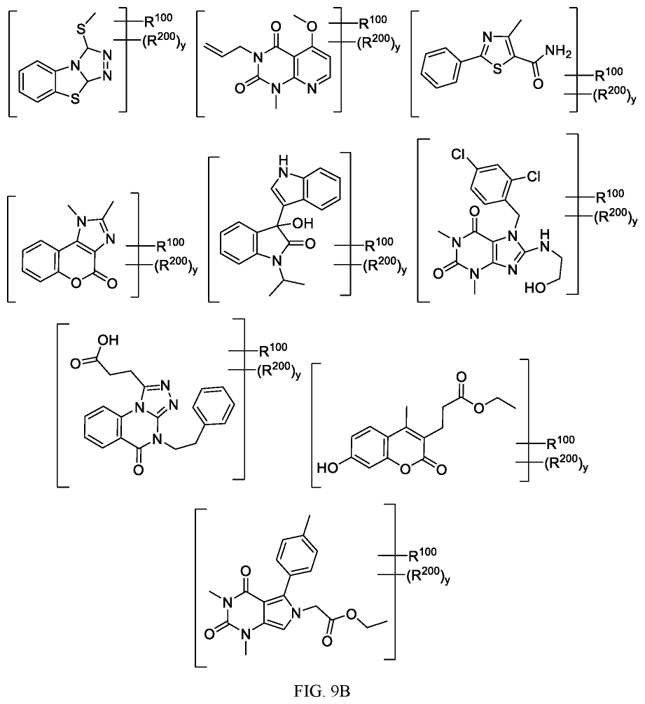

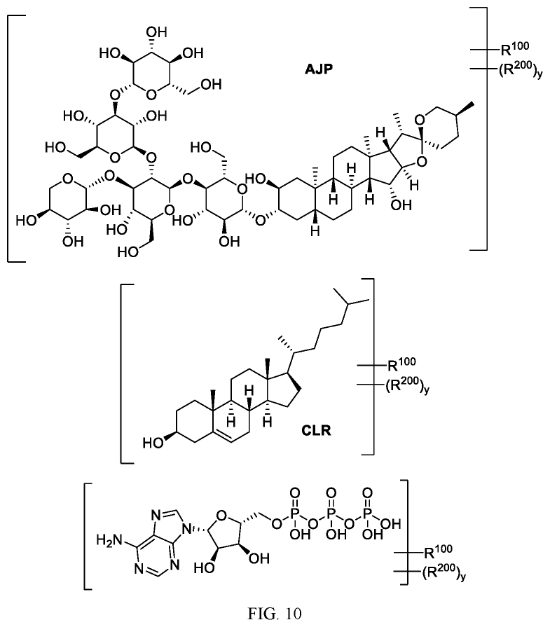

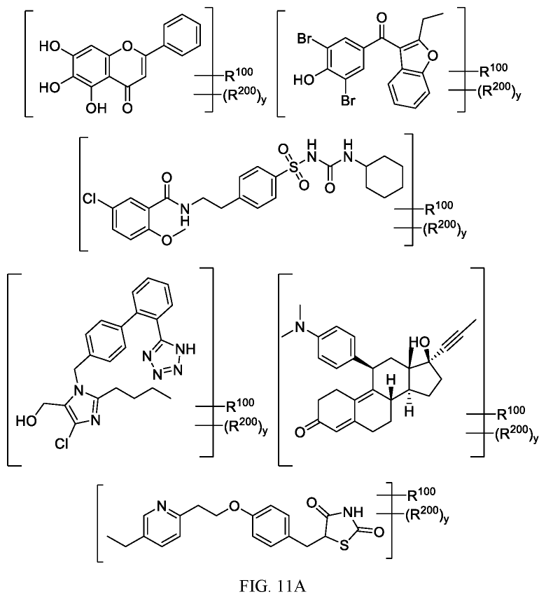

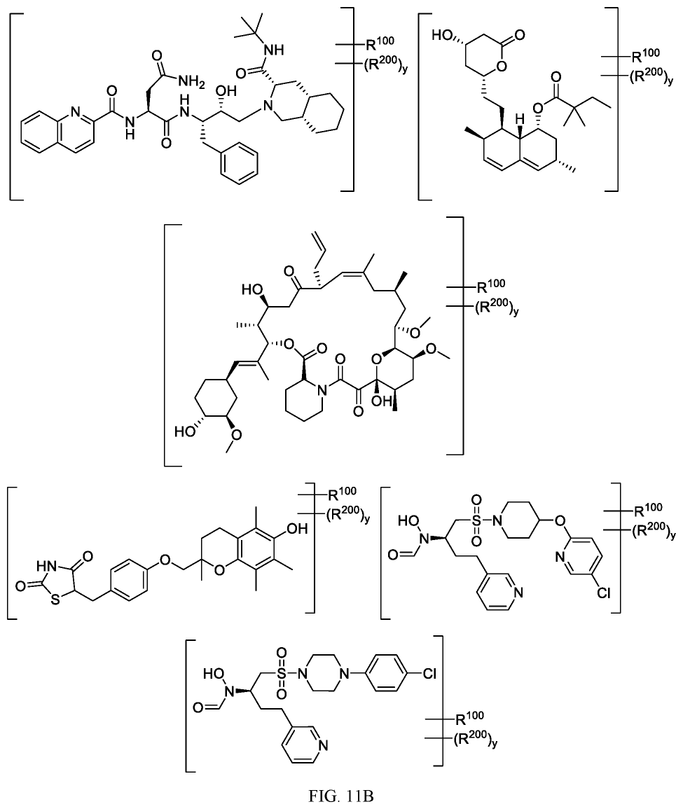

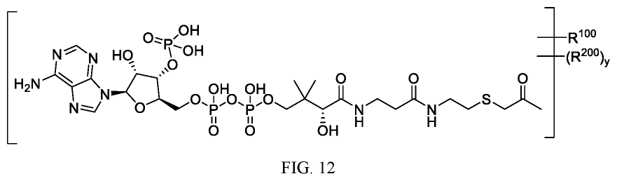

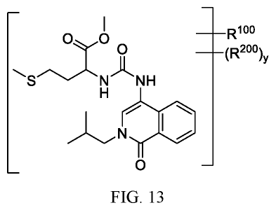

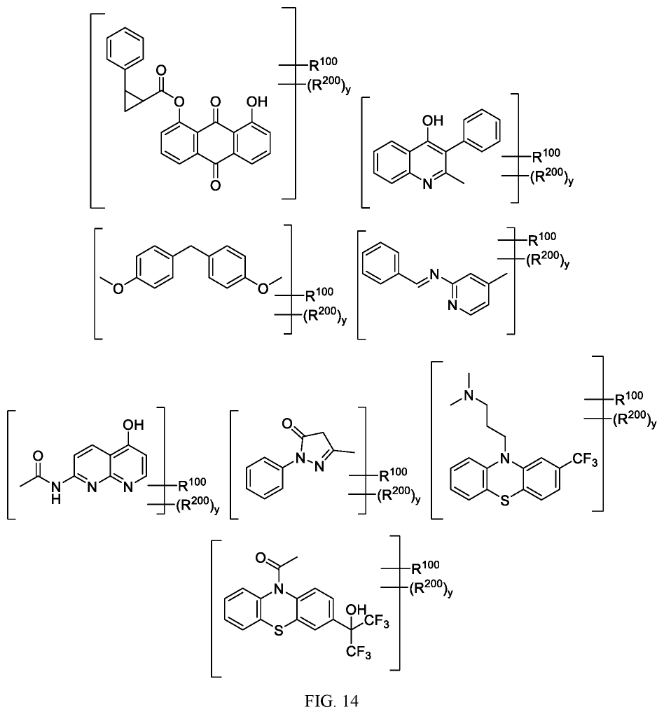

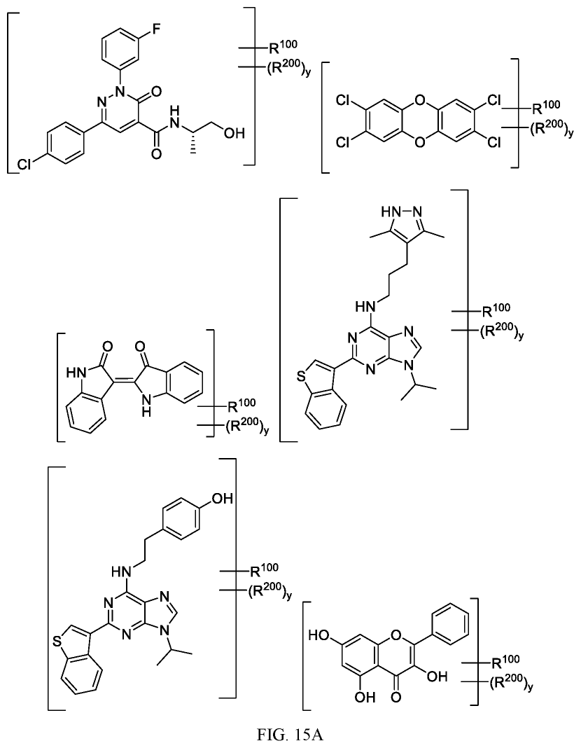

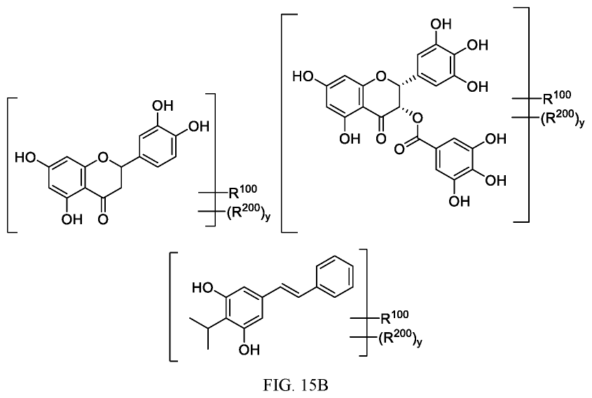

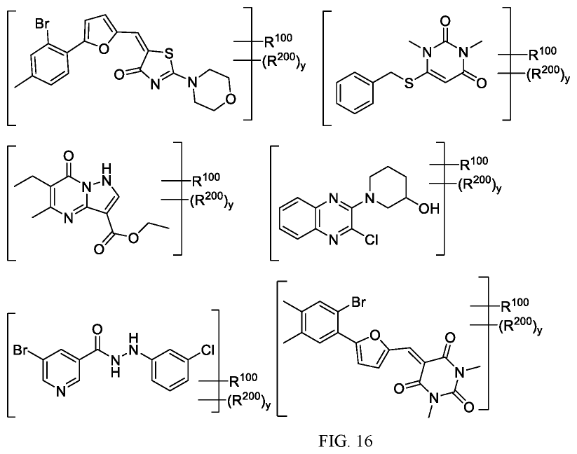

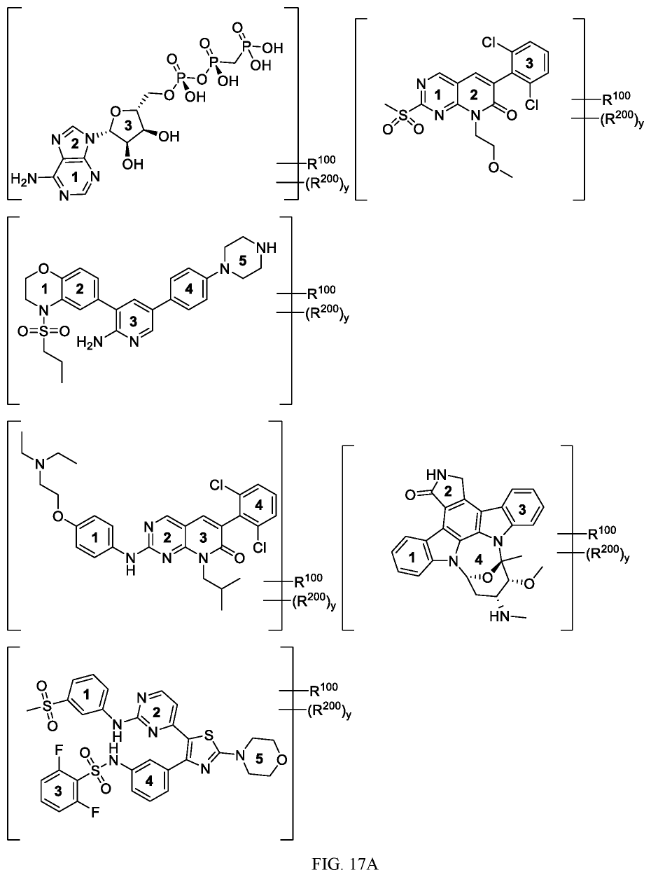

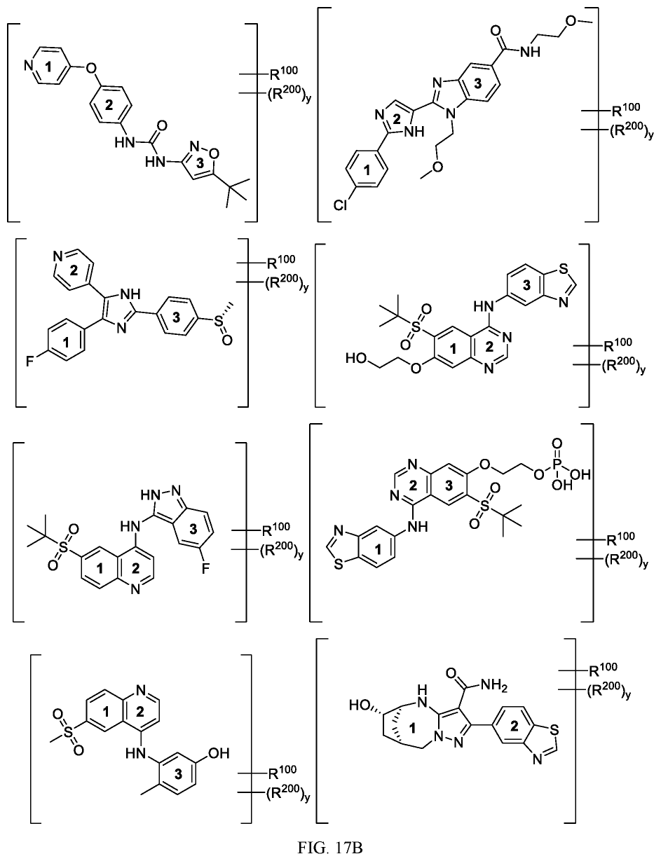

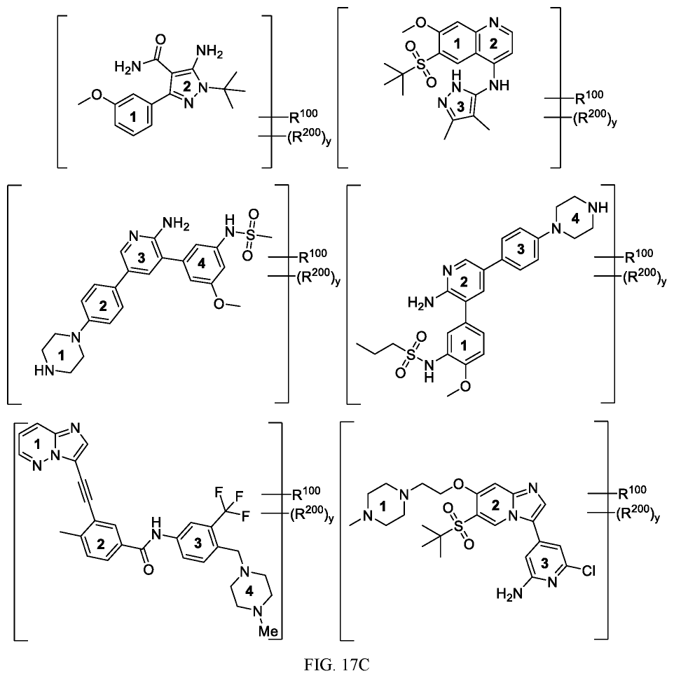

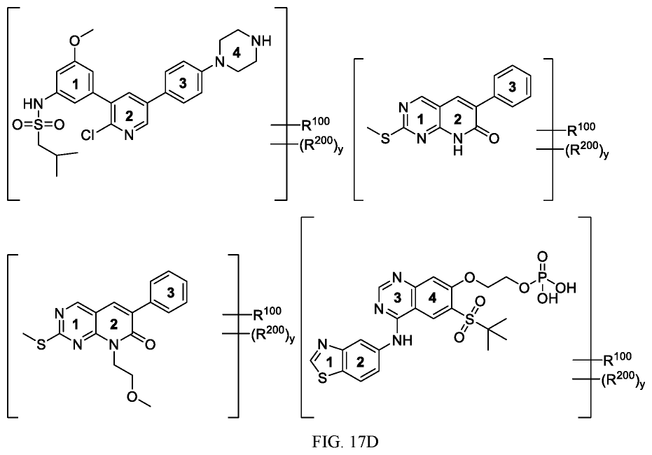

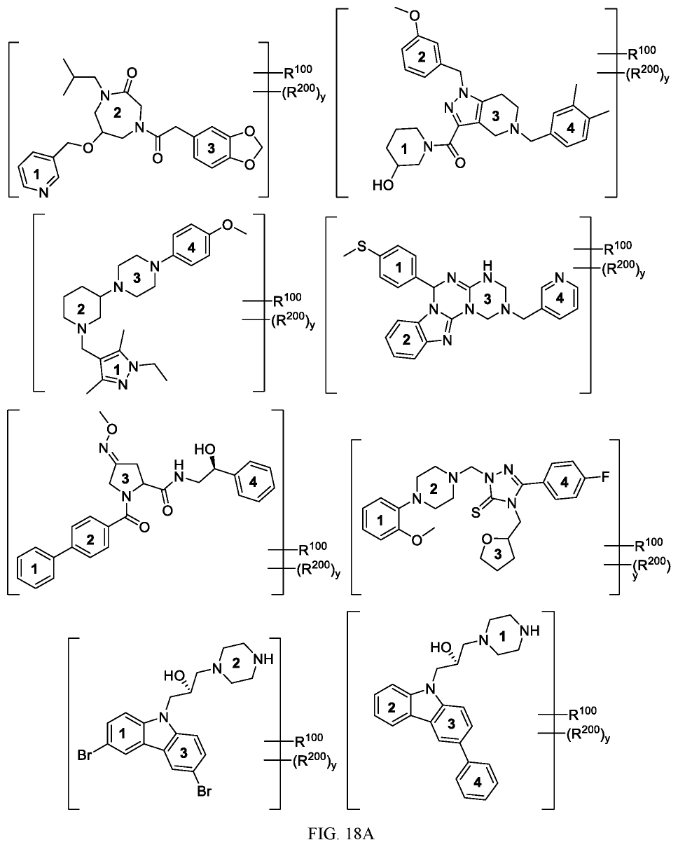

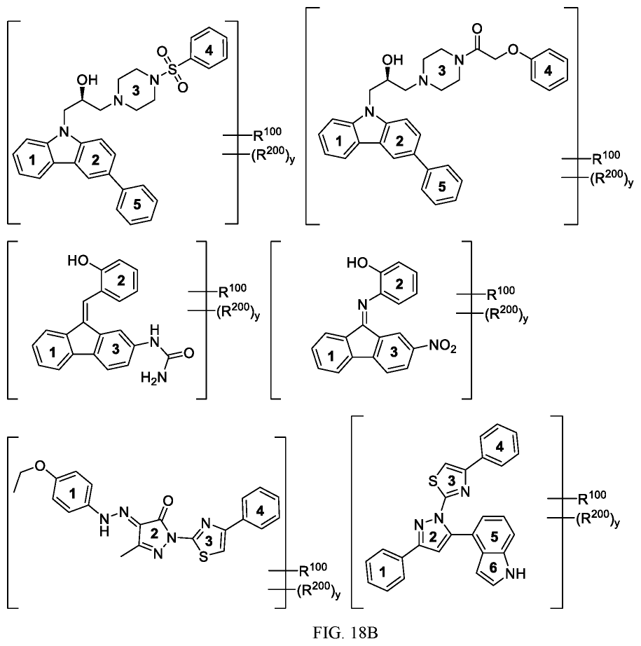

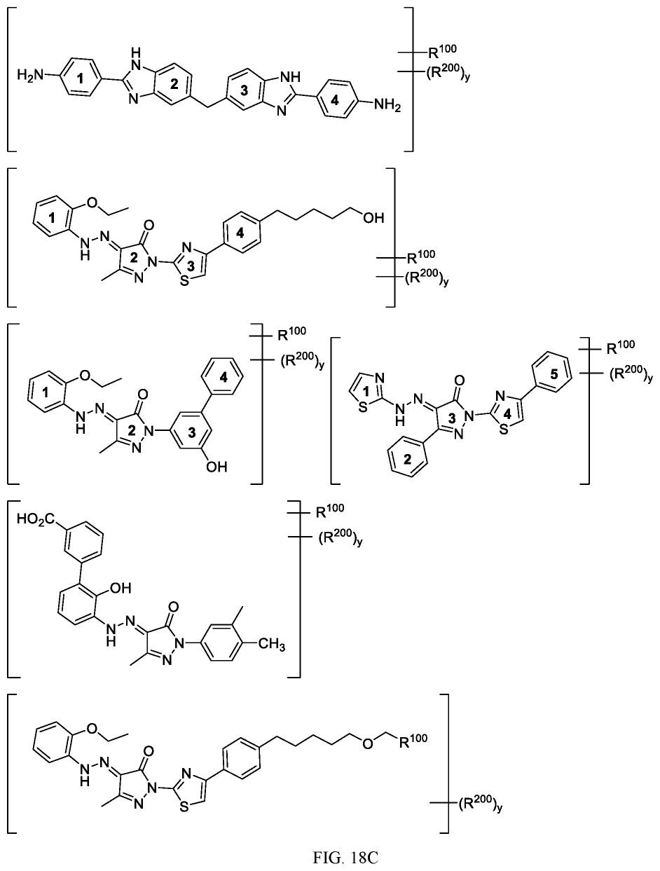

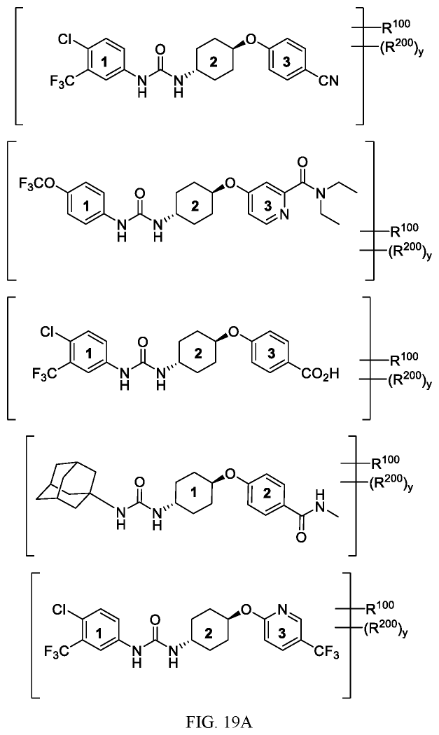

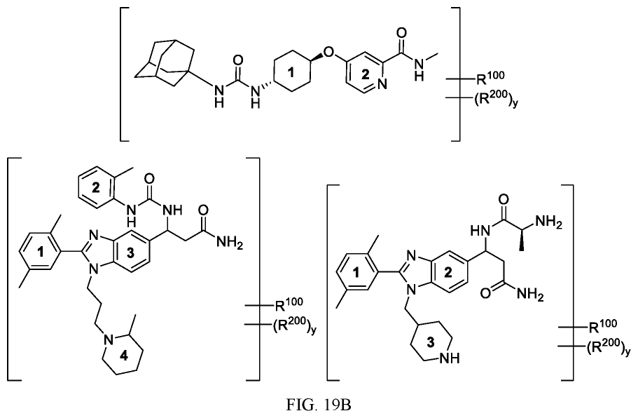

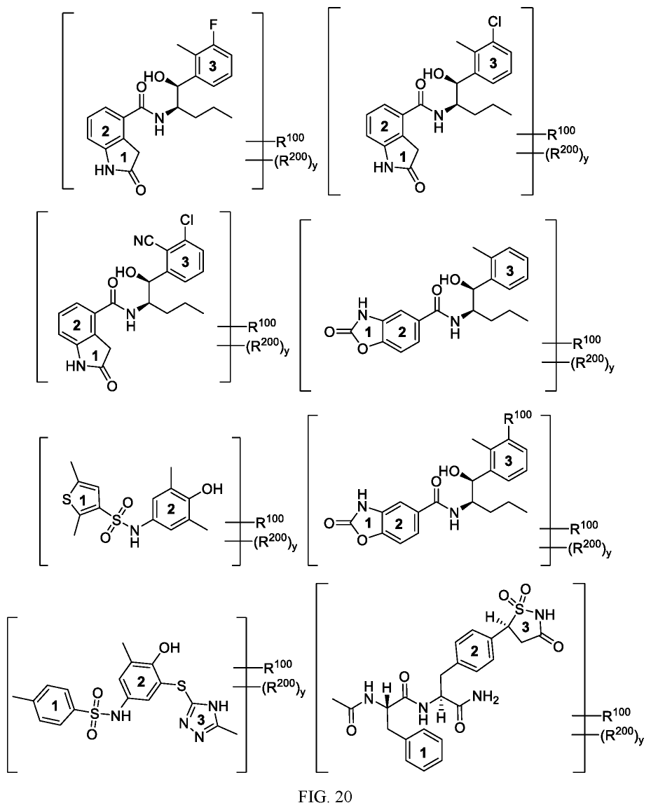

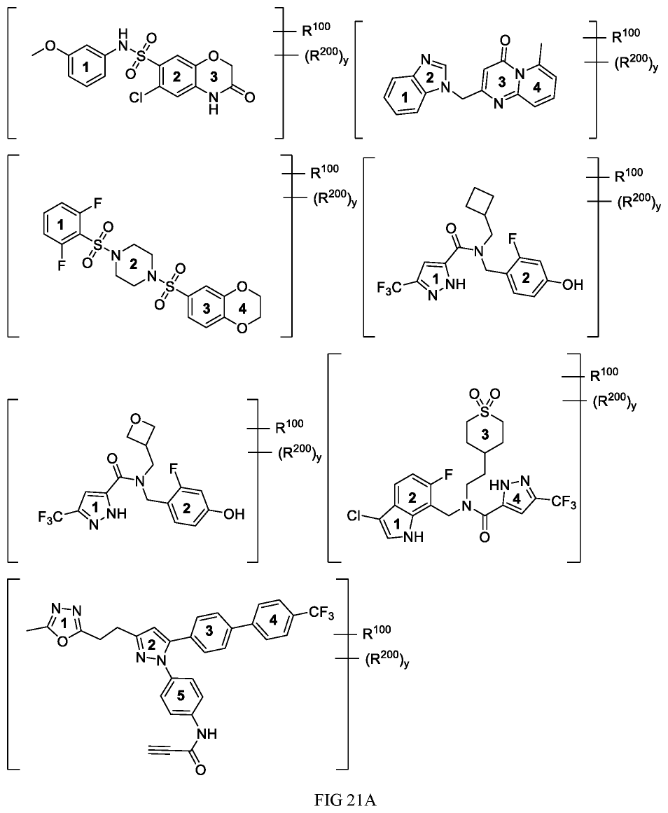

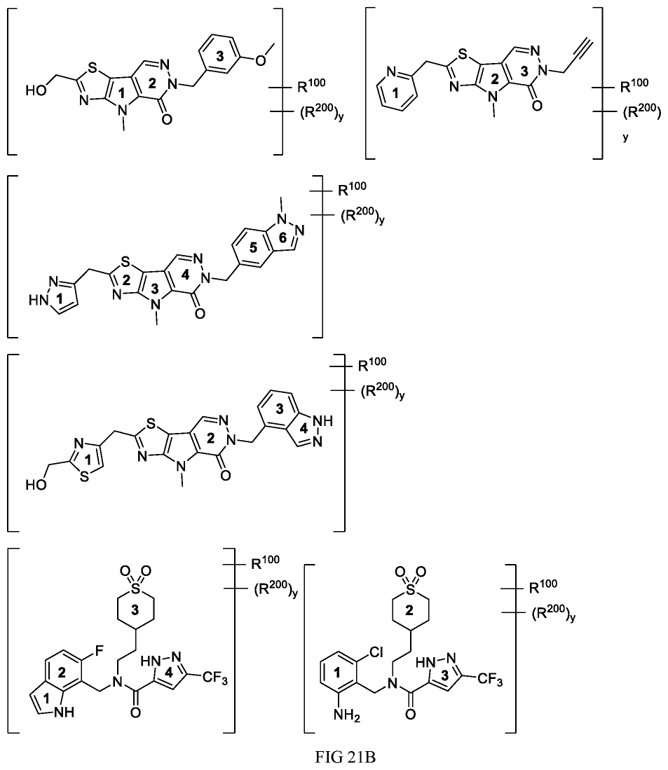

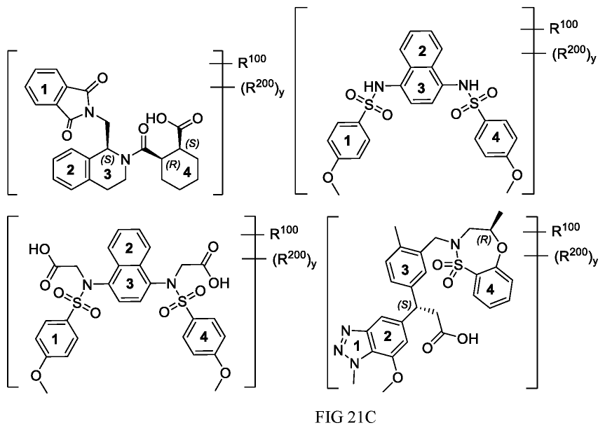

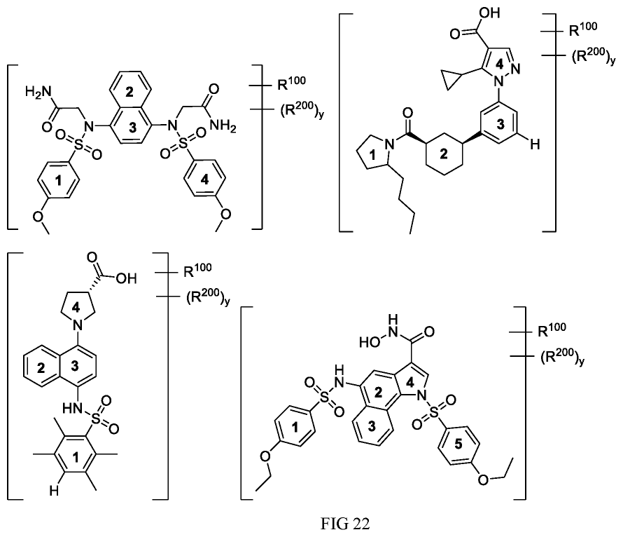

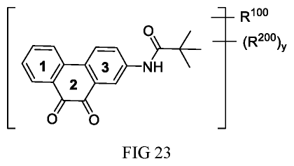

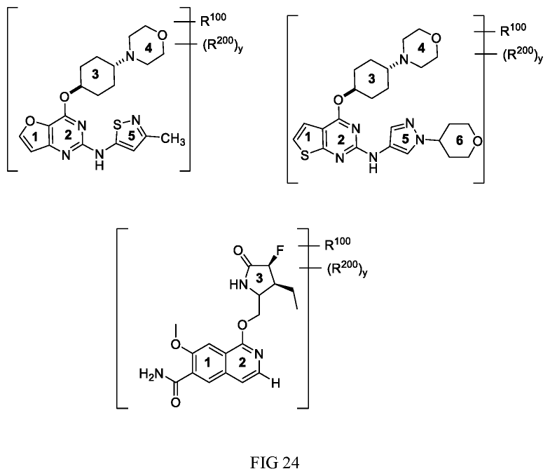

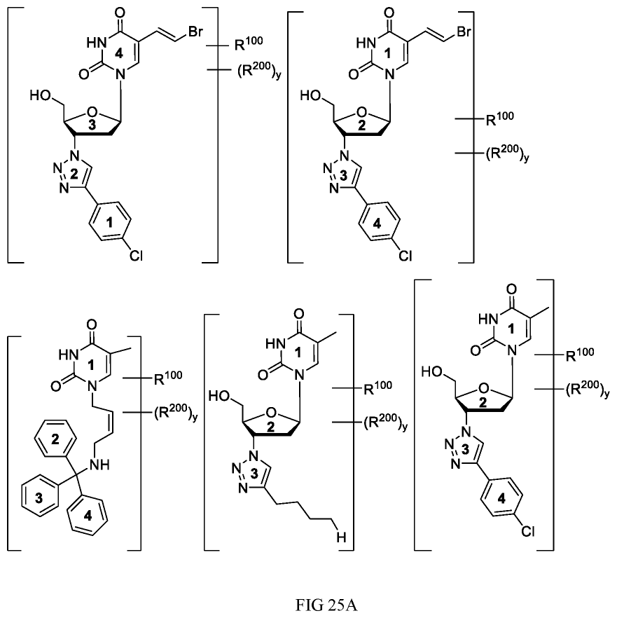

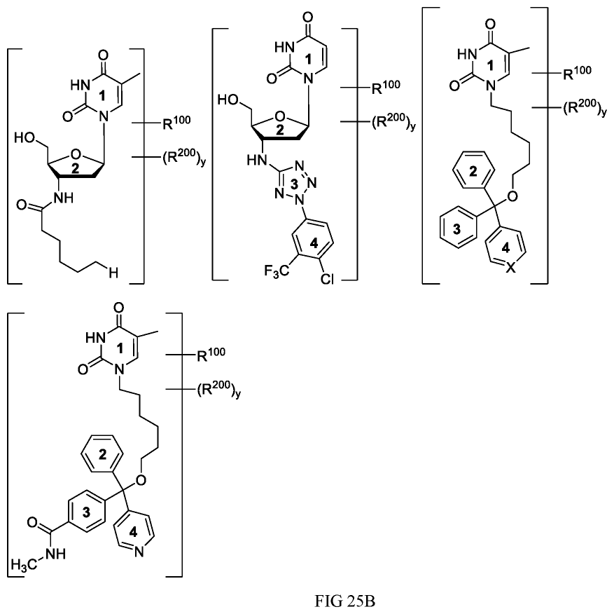

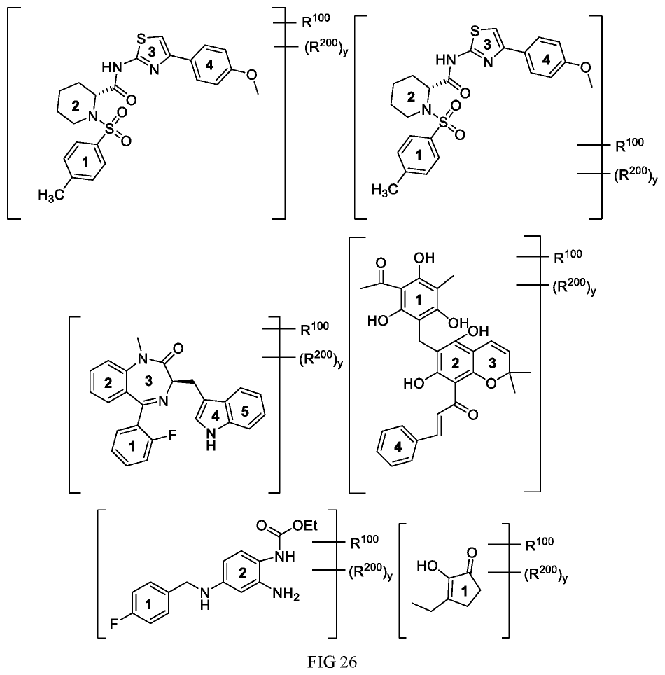

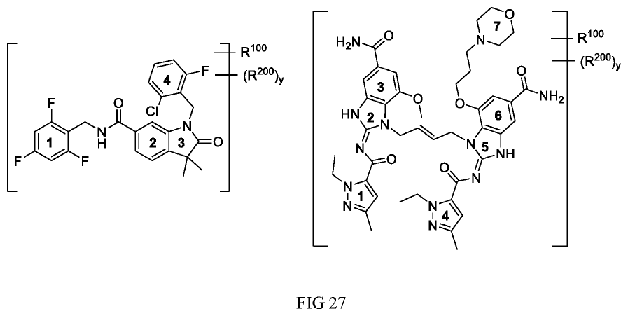

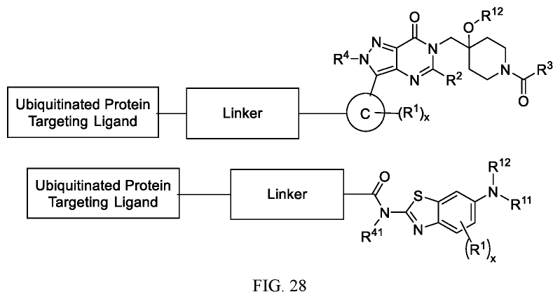

L₁, L₂, L₃, L₄, L₅, and L₆ are independently selected from the group consisting of a bond, alkyl, alkene, alkyne, haloalkyl, alkoxy, aryl, heterocycle, heteroaryl, bicycle, —C(O)—, —C(O)O—, —OC(O)—, —SO₂—, —S(O)—, —C(S)—, —C(O)NR¹¹—, —NR¹¹C(O)—, —O—, —S—, —NR¹¹—, —P(O)(OR¹¹)O—, —P(O)(OR¹¹)—, polyethylene glycol, lactic acid, and glycolic acid, each of which except bond is optionally substituted with 1, 2, 3, or 4 substituents independently selected from R⁴⁴; wherein L₁, L₂, L₃, L₄, L₅, and L₆ are selected such that there are no more than two of the same moieties connected together (e.g, L₁, L₂, and L₃ cannot all three be —C(O)—) and O and N atoms are not directly linked together except within aromatic rings (e.g. L₁ and L₂ cannot both be —O— or NR¹¹);
R⁴⁴ is independently selected at each instance from hydrogen, alkyl, haloalkyl, alkenyl, alkynyl, aryl, heterocycle, heteroaryl, amino, hydroxyl, alkoxy, —NR¹¹R¹², halogen, cyano, nitro, —OC(O)R⁴⁰, —NR¹¹C(O)R⁴⁰, —C(O)R⁴⁰, —OP(O)(R⁴⁰)₂, —P(O)(R⁴⁰)₂, —NR¹¹P(O)(R⁴⁰)₂, —SR¹¹, —OR¹¹, —S(O)R⁴⁰, —S(O)₂R⁴⁰, and —N(alkyl)C(O)R⁴⁰, each of which except hydrogen is optionally substituted as allowed by valence with 1, 2, 3, or 4 substituents selected from R⁴⁵; and
R⁴⁵ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl.
33. The compound of embodiment 31, wherein L₁ is bond.
34. The compound of embodiment 31, wherein L₁ is alkyl optionally substituted with 1, 2, 3, or 4 substituents independently selected from R⁴⁴.
35. The compound of embodiment 31, wherein L₁ is haloalkyl optionally substituted with 1, 2, 3, or 4 substituents independently selected from R⁴⁴.
36. The compound of embodiment 31, wherein L₁ is aryl optionally substituted with 1, 2, 3, or 4 substituents independently selected from R⁴⁴.
37. The compound of embodiment 31, wherein L₁ is heterocycle optionally substituted with 1, 2, 3, or 4 substituents independently selected from R⁴⁴.
38. The compound of embodiment 31, wherein L₁ is heteroaryl optionally substituted with 1, 2, 3, or 4 substituents independently selected from R⁴⁴.
39. The compound of embodiment 31, wherein L₁ is bicycle optionally substituted with 1, 2, 3, or 4 substituents independently selected from R⁴⁴.
40. The compound of embodiment 31, wherein L₁ is —C(O)—.
41. The compound of embodiment 31, wherein L₁ is —SO₂—.
42. The compound of embodiment 31, wherein L₁ is —C(O)O—, —OC(O)—, —NR¹¹C(O)—, and —C(O)NR¹¹—.
43. The compound of embodiment 31, wherein L₁ is —O—.
44. The compound of embodiment 31, wherein L₁ is —S—.
45. The compound of embodiment 31, wherein L₁ is —NR¹¹—.
46. The compound of embodiment 31, wherein L₁ is polyethylene glycol.
47. The compound of embodiment 31, wherein L₁ is lactic acid or glycolic acid.
48. The compound of any one of embodiments 31-47, wherein L₂ is bond.
49. The compound of any one of embodiments 31-47, wherein L₂ is alkyl optionally substituted with 1, 2, 3, or 4 substituents independently selected from R⁴⁴.
50. The compound of any one of embodiments 31-47, wherein L₂ is haloalkyl optionally substituted with 1, 2, 3, or 4 substituents independently selected from R⁴⁴.
51. The compound of any one of embodiments 31-47, wherein L₂ is aryl optionally substituted with 1, 2, 3, or 4 substituents independently selected from R⁴⁴.
52. The compound of any one of embodiments 31-47, wherein L₂ is heterocycle optionally substituted with 1, 2, 3, or 4 substituents independently selected from R⁴⁴.
53. The compound of any one of embodiments 31-47, wherein L₂ is heteroaryl optionally substituted with 1, 2, 3, or 4 substituents independently selected from R⁴⁴.
54. The compound of any one of embodiments 31-47, wherein L₂ is bicycle optionally substituted with 1, 2, 3, or 4 substituents independently selected from R⁴⁴.
55. The compound of any one of embodiments 31-47, wherein L₂ is polyethylene glycol.
56. The compound of any one of embodiments 31-47, wherein L₂ is lactic acid or glycolic acid.
57. The compound of any one of embodiments 31-56, wherein L₃ is bond.
58. The compound of any one of embodiments 31-56, wherein L₃ is alkyl optionally substituted with 1, 2, 3, or 4 substituents independently selected from R⁴⁴.
59. The compound of any one of embodiments 31-56, wherein L₃ is haloalkyl optionally substituted with 1, 2, 3, or 4 substituents independently selected from R⁴⁴.
60. The compound of any one of embodiments 31-56, wherein L₃ is aryl optionally substituted with 1, 2, 3, or 4 substituents independently selected from R⁴⁴.
61. The compound of any one of embodiments 31-56, wherein L₃ is heterocycle optionally substituted with 1, 2, 3, or 4 substituents independently selected from R⁴⁴.
62. The compound of any one of embodiments 31-56, wherein L₃ is heteroaryl optionally substituted with 1, 2, 3, or 4 substituents independently selected from R⁴⁴.
63. The compound of any one of embodiments 31-56, wherein L₃ is bicycle optionally substituted with 1, 2, 3, or 4 substituents independently selected from R⁴⁴.
64. The compound of any one of embodiments 31-56, wherein L₃ is —C(O)—.
65. The compound of any one of embodiments 31-56, wherein L₃ is —SO₂—.
66. The compound of any one of embodiments 31-56, wherein L₃ is —C(O)O—, —OC(O)—, —NR¹¹C(O)—, and —C(O)NR¹¹—.
67. The compound of any one of embodiments 31-56, wherein L₃ is —O—.
68. The compound of any one of embodiments 31-56, wherein L₃ is —S—.
69. The compound of any one of embodiments 31-56, wherein L₃ is —NR¹¹_.
70. The compound of any one of embodiments 31-56, wherein L₃ is polyethylene glycol.
71. The compound of any one of embodiments 31-56, wherein L₃ is lactic acid or glycolic acid.
72. The compound of any one of embodiments 31-71, wherein L₄ is bond.
73. The compound of any one of embodiments 31-71, wherein L₄ is alkyl optionally substituted with 1, 2, 3, or 4 substituents independently selected from R⁴⁴.
74. The compound of any one of embodiments 31-71, wherein L₄ is haloalkyl optionally substituted with 1, 2, 3, or 4 substituents independently selected from R⁴⁴.
75. The compound of any one of embodiments 31-71, wherein L₄ is aryl optionally substituted with 1, 2, 3, or 4 substituents independently selected from R⁴⁴.
76. The compound of any one of embodiments 31-71, wherein L₄ is heterocycle optionally substituted with 1, 2, 3, or 4 substituents independently selected from R⁴⁴.
77. The compound of any one of embodiments 31-71, wherein L₄ is heteroaryl optionally substituted with 1, 2, 3, or 4 substituents independently selected from R⁴⁴.
78. The compound of any one of embodiments 31-71, wherein L₄ is bicycle optionally substituted with 1, 2, 3, or 4 substituents independently selected from R⁴⁴.
79. The compound of any one of embodiments 31-71, wherein L₄ is polyethylene glycol.
80. The compound of any one of embodiments 31-71, wherein L₄ is lactic acid or glycolic acid.
81. The compound of any one of embodiments 31-80, wherein L₅ is bond.
82. The compound of any one of embodiments 31-80, wherein L₅ is alkyl optionally substituted with 1, 2, 3, or 4 substituents independently selected from R⁴⁴.
83. The compound of any one of embodiments 31-80, wherein L₅ is haloalkyl optionally substituted with 1, 2, 3, or 4 substituents independently selected from R⁴⁴.
84. The compound of any one of embodiments 31-80, wherein L₅ is aryl optionally substituted with 1, 2, 3, or 4 substituents independently selected from R⁴⁴.
85. The compound of any one of embodiments 31-80, wherein L₅ is heterocycle optionally substituted with 1, 2, 3, or 4 substituents independently selected from R⁴⁴.
86. The compound of any one of embodiments 31-80, wherein L₅ is heteroaryl optionally substituted with 1, 2, 3, or 4 substituents independently selected from R⁴⁴.
87. The compound of any one of embodiments 31-80, wherein L₅ is bicycle optionally substituted with 1, 2, 3, or 4 substituents independently selected from R⁴⁴.
88. The compound of any one of embodiments 31-80, wherein L₅ is —C(O)—.
89. The compound of any one of embodiments 31-80, wherein L₅ is —SO₂—.
90. The compound of any one of embodiments 31-80, wherein L₅ is —C(O)O—, —OC(O)—, —NR¹¹C(O)—, and —C(O)NR¹¹—.
91. The compound of any one of embodiments 31-80, wherein L₅ is —O—.
92. The compound of any one of embodiments 31-80, wherein L₅ is —S—.
93. The compound of any one of embodiments 31-80, wherein L₅ is —NR¹¹—.
94. The compound of any one of embodiments 31-80, wherein L₅ is polyethylene glycol.
95. The compound of any one of embodiments 31-80, wherein L₅ is lactic acid or glycolic acid.
96. The compound of any one of embodiments 31-95, wherein L₆ is bond.
97. The compound of any one of embodiments 31-95, wherein L₆ is alkyl optionally substituted with 1, 2, 3, or 4 substituents independently selected from R⁴⁴.
98. The compound of any one of embodiments 31-95, wherein L₆ is haloalkyl optionally substituted with 1, 2, 3, or 4 substituents independently selected from R⁴⁴.
99. The compound of any one of embodiments 31-95, wherein L₆ is aryl optionally substituted with 1, 2, 3, or 4 substituents independently selected from R⁴⁴.
100. The compound of any one of embodiments 31-95, wherein L₆ is heterocycle optionally substituted with 1, 2, 3, or 4 substituents independently selected from R⁴⁴.
101. The compound of any one of embodiments 31-95, wherein L₆ is heteroaryl optionally substituted with 1, 2, 3, or 4 substituents independently selected from R⁴⁴.
102. The compound of any one of embodiments 31-95, wherein L₆ is bicycle optionally substituted with 1, 2, 3, or 4 substituents independently selected from R⁴⁴.
103. The compound of any one of embodiments 31-95, wherein L₆ is polyethylene glycol.
104. The compound of any one of embodiments 31-95, wherein L₆ is lactic acid or glycolic acid.
105. The compound of any one of embodiments 31-95, wherein L₁ is bound to USP7 Targeting Ligand.
106. The compound of any one of embodiments 31-95, wherein L₁ is bound to Ubiquitinated Protein Targeting Ligand.
107. The compound of any one of embodiments 31-106, wherein R⁴⁴ is independently selected at each instance from alkyl, halogen, and haloalkyl.
108. The compound of any one of embodiments 31-106, wherein R⁴⁴ is alkyl optionally substituted as allowed by valence with 1, 2, 3, or 4 substituents selected from R⁴⁵.
109. The compound of any one of embodiments 31-106, wherein R⁴⁴ is aryl optionally substituted as allowed by valence with 1, 2, 3, or 4 substituents selected from R⁴⁵.
110. The compound of any one of embodiments 31-106, wherein R⁴⁴ is heterocycle optionally substituted as allowed by valence with 1, 2, 3, or 4 substituents selected from R⁴⁵.
111. The compound of any one of embodiments 31-106, wherein R⁴⁴ is heteroaryl optionally substituted as allowed by valence with 1, 2, 3, or 4 substituents selected from R⁴⁵.
112. The compound of any one of embodiments 31-106, wherein R⁴⁴ is amino optionally substituted as allowed by valence with 1, 2, 3, or 4 substituents selected from R⁴⁵.
113. The compound of any one of embodiments 31-106, wherein R⁴⁴ is hydroxyl optionally substituted as allowed by valence with 1, 2, 3, or 4 substituents selected from R⁴⁵.
114. The compound of any one of embodiments 31-106, wherein R⁴⁴ is alkoxy optionally substituted as allowed by valence with 1, 2, 3, or 4 substituents selected from R⁴⁵.
115. The compound of any one of embodiments 31-114, wherein R⁴⁵ is independently selected from halogen, alkyl, and haloalkyl.
116. The compound of any one of embodiments 31-114, wherein R⁴⁵ is independently selected from amino, hydroxyl, alkoxy, —NHalkyl, —N(alkyl)₂, —OC(O)alkyl, —NHC(O)alkyl, and —N(alkyl)C(O)alkyl.
117. The compound of any one of embodiments 1-116, wherein the Ubiquitinated Protein Targeting Ligand is a ligand that binds CFTR.
118. The compound of embodiment 117, wherein the Ubiquitinated Protein Targeting Ligand is selected from FIG. 2A, FIG. 2B, FIG. 2C, and FIG. 2D.
119. The compound of any one of embodiments 1-116, wherein the Ubiquitinated Protein Targeting Ligand is a ligand that binds phenylalanine hydroxylase.
120. The compound of embodiment 119, wherein the Ubiquitinated Protein Targeting Ligand is selected from FIG. 3A, FIG. 3B, and FIG. 3C.
121. The compound of any one of embodiments 1-116, wherein the Ubiquitinated Protein Targeting Ligand is a ligand that binds p53.
122. The compound of embodiment 121, wherein the Ubiquitinated Protein Targeting Ligand is selected from FIG. 4A, FIG. 4B, and FIG. 4C.
123. The compound of any one of embodiments 1-116, wherein the Ubiquitinated Protein Targeting Ligand is a ligand that binds rhodopsin.
124. The compound of embodiment 123, wherein the Ubiquitinated Protein Targeting Ligand is selected from FIG. 5A and FIG. 5B.
125. The compound of any one of embodiments 1-116, wherein the Ubiquitinated Protein Targeting Ligand is a ligand that binds c-myc.
126. The compound of embodiment 125, wherein the Ubiquitinated Protein Targeting Ligand is selected from FIG. 6A and FIG. 6B.
127. The compound of any one of embodiments 1-116, wherein the Ubiquitinated Protein Targeting Ligand is a ligand that binds RIPK1.
128. The compound of embodiment 127, wherein the Ubiquitinated Protein Targeting Ligand is selected from FIG. 7A, FIG. 7B, FIG. 7C, FIG. 7D, and FIG. 7E.
129. The compound of any one of embodiments 1-116, wherein the Ubiquitinated Protein Targeting Ligand is a ligand that binds RIPK1.
130. The compound of embodiment 129, wherein the Ubiquitinated Protein Targeting Ligand is selected from FIG. 8.
131. The compound of any one of embodiments 1-116, wherein the Ubiquitinated Protein Targeting Ligand is a ligand that binds CDKN1B.
132. The compound of embodiment 131, wherein the Ubiquitinated Protein Targeting Ligand is selected from FIG. 9A and FIG. 9B.
133. The compound of any one of embodiments 1-116, wherein the Ubiquitinated Protein Targeting Ligand is a ligand that binds ABCA4.
134. The compound of embodiment 133, wherein the Ubiquitinated Protein Targeting Ligand is selected from FIG. 10.
135. The compound of any one of embodiments 1-116, wherein the Ubiquitinated Protein Targeting Ligand is a ligand that binds ABCB11.
136. The compound of embodiment 136, wherein the Ubiquitinated Protein Targeting Ligand is selected from FIG. 11A and FIG. 11B.
137. The compound of any one of embodiments 1-116, wherein the Ubiquitinated Protein Targeting Ligand is a ligand that binds choline acetylase.
138. The compound of embodiment 137, wherein the Ubiquitinated Protein Targeting Ligand is selected from FIG. 12.
139. The compound of any one of embodiments 1-116, wherein the Ubiquitinated Protein Targeting Ligand is a ligand that binds CYLD.
140. The compound of embodiment 139, wherein the Ubiquitinated Protein Targeting Ligand is selected from FIG. 13.
141. The compound of any one of embodiments 1-116, wherein the Ubiquitinated Protein Targeting Ligand is a ligand that binds NEMO.
142. The compound of embodiment 141, wherein the Ubiquitinated Protein Targeting Ligand is selected from FIG. 14.
143. The compound of any one of embodiments 1-116, wherein the Ubiquitinated Protein Targeting Ligand is a ligand that binds AH receptor-interacting protein.
144. The compound of embodiment 143, wherein the Ubiquitinated Protein Targeting Ligand is selected from FIG. 15A and FIG. 15B.
145. The compound of any one of embodiments 1-116, wherein the Ubiquitinated Protein Targeting Ligand is a ligand that binds PDCD4.
146. The compound of embodiment 145, wherein the Ubiquitinated Protein Targeting Ligand is selected from FIG. 16.
147. The compound of any one of embodiments 1-116, wherein the Ubiquitinated Protein Targeting Ligand is a ligand that binds RIPK2.
148. The compound of embodiment 147, wherein the Ubiquitinated Protein Targeting Ligand is selected from FIG. 17A, FIG. 17B, FIG. 17C, and FIG. 17D.
149. The compound of any one of embodiments 1-116, wherein the Ubiquitinated Protein Targeting Ligand is a ligand that binds BAX.
150. The compound of embodiment 149, wherein the Ubiquitinated Protein Targeting Ligand is selected from FIG. 18A, FIG. 18B, and FIG. 18C.
151. The compound of any one of embodiments 1-116, wherein the Ubiquitinated Protein Targeting Ligand is a ligand that binds P21.
152. The compound of embodiment 151, wherein the Ubiquitinated Protein Targeting Ligand is selected from FIG. 19A and FIG. 19B.
153. The compound of any one of embodiments 1-116, wherein the Ubiquitinated Protein Targeting Ligand is a ligand that binds SERPINA1.
154. The compound of embodiment 153, wherein the Ubiquitinated Protein Targeting Ligand is selected from FIG. 20.
155. The compound of any one of embodiments 1-116, wherein the Ubiquitinated Protein Targeting Ligand is a ligand that binds PKLR.
156. The compound of embodiment 155, wherein the Ubiquitinated Protein Targeting Ligand is selected from FIG. 21A, FIG. 21B, and FIG. 21C.
157. The compound of any one of embodiments 1-116, wherein the Ubiquitinated Protein Targeting Ligand is a ligand that binds KEAP1.
158. The compound of embodiment 157, wherein the Ubiquitinated Protein Targeting Ligand is selected from FIG. 22.
159. The compound of any one of embodiments 1-116, wherein the Ubiquitinated Protein Targeting Ligand is a ligand that binds PTEN.
160. The compound of embodiment 159, wherein the Ubiquitinated Protein Targeting Ligand is selected from FIG. 23.
161. The compound of any one of embodiments 1-116, wherein the Ubiquitinated Protein Targeting Ligand is a ligand that binds IRAK4.
162. The compound of embodiment 161, wherein the Ubiquitinated Protein Targeting Ligand is selected from FIG. 24.
163. The compound of any one of embodiments 1-116, wherein the Ubiquitinated Protein Targeting Ligand is a ligand that binds TK2.
164. The compound of embodiment 163, wherein the Ubiquitinated Protein Targeting Ligand is selected from FIG. 25A and FIG. 25B.
165. The compound of any one of embodiments 1-116, wherein the Ubiquitinated Protein Targeting Ligand is a ligand that binds KCNQ1.
166. The compound of embodiment 165, wherein the Ubiquitinated Protein Targeting Ligand is selected from FIG. 26.
167. The compound of any one of embodiments 1-116, wherein the Ubiquitinated Protein Targeting Ligand is a ligand that binds STING1.
168. The compound of embodiment 167, wherein the Ubiquitinated Protein Targeting Ligand is selected from FIG. 27.
169. A pharmaceutical composition comprising an effective amount of a compound of any one of embodiments 1-168 or a pharmaceutically acceptable salt thereof and a pharmaceutically acceptable carrier.
170. A method of treating a disorder mediated by the Target Ubiquitinated Protein in a human comprising administering an effective amount of a compound or a pharmaceutically acceptable salt thereof of any one of embodiments 1-168.
171. A compound of any one of embodiments 1-168 or a pharmaceutically acceptable salt thereof for use in the manufacture of a medicament for the treatment of a disorder mediated by the Target Ubiquitinated Protein.
172. Use of a compound of any one of embodiments 1-168 or a pharmaceutically acceptable salt thereof in the treatment of a disorder mediated by the Target Ubiquitinated Protein in a human.
173. A pharmaceutical composition that comprises an effective amount of a compound of any one of embodiments 1-168 or a pharmaceutically acceptable salt thereof for use in the treatment of a disorder mediated by the Target Ubiquitinated Protein in a human optionally with a pharmaceutically acceptable carrier.

### Protein Function Restoration Assays

In certain embodiments a method of stabilizing and restoring a protein's function is provided. The skilled artisan will recognize how to assess whether or not a protein's function has been restored in vivo or in vitro depending on context. For example, when the Target Ubiquitinated Protein is an ion channel, such as CFTR, surface representation assays or ion current assays can be used to assay protein function restoration in vitro. Additionally, a reduction of symptoms associated with a disease mediated by the Target Ubiquitinated Protein will show in vivo efficacy. For example, when the Target Ubiquitinated Protein is CFTR amelioration of cystic fibrosis symptoms will result from protein function restoration in vivo. When the Target Ubiquitinated Protein is an oncological target, such as p53, cell death assays or cell cycle assays can be used to demonstrate the restoration of function. When the Target Ubiquitinated Protein is an enzyme then its enzymatic activity can be assayed to demonstrate the restoration of function. Non-limiting examples of these assays are provided below.

### Ubiquitination Status Assays

The degree of deubiquitination of a protein target of interest in a cell upon treatment with varying concentrations of a compound of the current invention can be assessed. Briefly, cells that express the target of interest and that have been treated with varying concentrations of a compound of the current invention will be washed once with PBS without Ca²⁺, harvested, and resuspended in RIPA lysis buffer containing (in mM) Tris (20, pH 7.4), EDTA (1), NaCl (150), 0.1% (wt/vol) SDS, 1% Triton X-100, 1% sodium deoxycholate and supplemented with protease inhibitor mixture (10 μL/mL, Sigma-Aldrich), PMSF (1 mM, Sigma-Aldrich), N-ethylmaleimide (2 mM, Sigma-Aldrich) and PR-619 deubiquitinase inhibitor (50 μM, LifeSensors). Lysates will be prepared by incubation at 4° C. for 1 hr, with occasional vortex, and cleared by centrifugation (10,000×g, 10 min, 4° C.). Supernatants will be transferred to new tubes, with aliquots removed for quantification of total protein concentration determined by the bis-cinchonic acid protein estimation kit (Pierce Technologies). Lysates will be pre-cleared by incubation with 10 μL Protein A/G Sepharose beads (Rockland) for 40 min at 4° C. and then incubated with 0.75 μg anti-Q1 antibody (Alomone) for 1 hr at 4° C. Equivalent total protein amounts will be added to spin-columns containing 25 μL Protein A/G Sepharose beads, tumbling overnight at 4° C. Equivalent total protein amounts of pre-cleared lysates for the target of interest pulldowns will be added directly to 20 μL RFP-Trap conjugated agarose beads (Chromotek, rta-20), tumbling overnight at 4° C. Immunoprecipitates will be washed twice with RIPA buffer, 3 times with high salt RIPA (500 mM NaCl), spun down at 500×g, and eluted with 40 μL of warmed sample buffer [50 mM Tris, 10% (vol/vol) glycerol, 2% SDS, 100 mM DTT, and 0.2 mg/mL bromophenol blue], and boiled (55° C., 15 min). Proteins will be resolved on a 4-12% Bis Tris gradient precast gel (Life Technologies) in Mops-SDS running buffer (Life Technologies) at 200 V constant for ˜1 h. Protein bands will be transferred by tank transfer onto a nitrocellulose membrane in transfer buffer (25 mM Tris pH 8.3, 192 mM glycine, 15% (vol/vol) methanol, and 0.1% SDS). The membranes will be blocked with a solution of 5% nonfat milk in tris-buffered saline-tween (TBS-T) (25 mM Tris pH 7.4, 150 mM NaCl, and 0.1% Tween-20) for 1 hr at RT and then incubated overnight at 4° C. with primary antibodies against the target of interest in blocking solution. The blots will be washed with TBS-T three times for 10 min each and then incubated with secondary horseradish peroxidase-conjugated antibody for 1 hr at RT. After washing in TBS-T, the blots will be developed with a chemiluminiscent detection kit (Pierce Technologies) and then visualized on a gel imager. Membranes can then be stripped with harsh stripping buffer (2% SDS, 62 mM Tris pH 6.8, 0.8% β-mercaptoethanol) at 50° C. for 30 min, rinsed under running water for 2 min, and washed with TBST (3×, 10 min). Membranes can then be pre-treated with 0.5% glutaraldehyde and re-blotted with an anti-ubiquitin antibody (LifeSensors VU1, 1:500) to assess the effect of a compound of the current invention treatment on the amount of ubiquitin present on the target.

### Protein Stabilization Assays

### I. Cell Line Overview

HiBiT Stable Cell Lines are generated by using site-specific insertion via CRISPR-Cas9 to fuse the 11-amino-acid HiBiT peptide tag to either the N′ or C′ terminus of the protein of interest (POI) depending on factors such as success of tagged POI expression or tag location (intracellular vs. extracellular side of a membrane protein). POI may include but are not limited to intracellular or intramembrane proteins. In the case of heterologous cells (i.e. HEK293), the HiBiT Stable Cell Line may also stably express intracellular NanoLuc luciferase-based LgBiT protein. The HiBiT and LgBiT proteins, when combined, reconstitute the active NanoBiT luciferase enzyme, which emits a luminescent signal in the presence of substrate (i.e. Nano-Glo Live Cell furimazine-based substrates). Stable Cells may stably express the HiBiT protein as a pool of cells or as a single clone (heterozygous or homozygous expression depending on target).

### II. HiBiT Kinetic Assay Protocol to Determine Protein Stabilization

The following protocol describes a high throughput assay capable of screening multiple compounds at several doses on a HiBiT-tagged POI.
1. HiBiT cell lines are plated up to 1 day prior to the assay in a tissue-culture-treated white 96 well plate with a lid using 100 μl DMEM+8% FBS+1% penicillin/streptomycin/glutamine media/well at a cell density of 5-20 k cells/well.
2. The following day, cells are equilibrated for 2.5 hours with 1× Nano-Glo Endurazine Live Cell substrate (50 μl/well) in CO₂ independent media+8% FBS+1% penicillin/streptomycin/glutamine to generate a stable background luminescent signal.
3. Cycloheximide is added at 2× concentration (i.e. 200 μM) in 50 μl/well to achieve a final 100 μM per well. For dose response measurement of compounds, suitable stock solutions are prepared at desired concentrations and are added concomitantly with the cycloheximide treatment.
4. Well Plates with cells are immediately moved to a plate reader capable of measuring luminescence with temperature set at 37° C. (e.g. Promega Glomax).
5. Luminescence signal is measured at 1-3 time points *optimized to the POI to observe differences in protein levels. At the final time point, cells are assessed for compound toxicity via CellTiter-Glo (see separate protocol).
6. Raw Data is converted to fold change over DMSO control at the specific time point and normalized with cell viability data to account for protein levels that may change with cell viability.
7. Compounds are selected for a secondary screen if protein levels from co-treatment with cycloheximide are significantly higher than that of with cycloheximide-only treatment.
8. Cells treated with compounds in a secondary screen (follow Protocol item 1-4) are assessed over a continuous time course as the cells are incubated in compound, with an integration time of 0.5-2 seconds every 1-2 hrs for 24-72 hrs (depending on half-life of assayed POI).
9. Raw Data is converted to fold change over DMSO control at the specific time point and plotted as a one phase decay plot. Half life calculations of the POI are determined based on the decay plot and compared between cycloheximide alone (steady-state POI degradation) cell treatment and cell treatment with cycloheximide plus the compound. Compounds that significantly extend the half-life of the POI are considered to stabilize the POI by deubiquitination from the recruited DUB.
   *NOTE: optimization of this time point is based on running a continuous 24-72 hr kinetic assay on the POI using cycloheximide, which generates data on protein half life. Each new target must be assessed initially in a cycloheximide chase screen before running the compound screen.

### Ion Channel Function Assays

Cell surface and total ion channel pools will be assayed by flow cytometry in live, transfected HEK293 cells that are treated with varying concentrations of compounds. 48 hrs post-transfection, cells cultured in 12-well plates will be gently washed with ice cold PBS containing Ca²⁺ and Mg²⁺ (in mM: 0.9 CaCl₂, 0.49 MgCl₂, pH 7.4), and incubated for 30 min in blocking medium (DMEM with 3% BSA) at 4° C. HEK293 cells expressing the ion channel of import will then be incubated with 1 μM Alexa Fluor 647 conjugated α-bungarotoxin (BTX₆₄₇; Life Technologies) in DMEM13% BSA on a rocker at 4° C. for 1 hr, followed by washing three times with PBS (containing Ca²⁺ and Mg²⁺). Cells will be harvested in Ca²⁺-free PBS, and assayed by flow cytometry. CFP- and YFP-tagged proteins are excited at 405 and 488 nm, respectively, and Alexa Fluor 647 is excited at 633 nm. The amount of ion channel at the surface (strength of fluorescent signal with Alexa Fluor 647) will be compared across the cell samples treated with differing amounts of compounds.

To measure the functional restoration of ion channels upon compound treatment electrophysiology experiments will be performed. For potassium channel measurements, whole-cell membrane currents will be recorded at room temperature in CHO cells using a patch-clamp amplifier. A coverslip with adherent CHO cells will be placed on the glass bottom of a recording chamber (0.7-1 mL in volume) mounted on the stage of an inverted microscope. An internal solution containing (mM): 133 KCl, 0.4 GTP, 10 EGTA, 1 MgSO₄, 5 K₂ATP, 0.5 CaCl₂, and 10 HEPES (pH 7.2) and an external solution containing (in mM): 147 NaCl, 4 KCl, 2 CaCl₂, and 10 HEPES (pH 7.4) will be used. Pipette resistance will be typically 1.5 MΩ when filled with the internal solution. I-V curves will be generated from a family of step depolarizations (−40 to +100 mV in 10 mV steps from a holding potential of −80 mV). Currents will be sampled at 20 kHz and filtered at 5 kHz. Traces will be acquired at a repetition interval of 10 s.

For whole-cell recordings of cardiomyocytes (KCQN1 target), they will be performed 48-72 hrs after expression of the channel and treatment with the compounds. The same internal and external solutions as are being used above will be used for the experiments. A slow voltage ramp protocol (from −80 my to +100 mV over 2 s) will be used to evoke whole-cell currents. Action potential recordings under current clamp will be obtained via 0.25 Hz stimulation with short current pulses (150 pA. 10 ms).

For CFTR channel measurements, whole-cell recordings will be carried out in HEK293 and FRT cells at room temperature. An internal solution containing (mM): 113 L-aspartic acid, 113 CsOH, 27 CsCl, 1 NaCl, 1 MgCl₂, 1 EGTA, 10 TES, 3 MgATP (pH 7.2) and an external solution containing (in mM): 145 NaCl, 4 CsCl, 1 CaCl₂, 1 MgCl₂, 10 glucose, and 10 TES (pH 7.4) will be used for the experiments. I-V curves will be generated from a family of step depolarizations (−80 to +80 mV in 20 mV steps from a holding potential of −40 mV). CFTR currents are activated by perfusion with 10 μM forskolin. In experiments utilizing VX809 (3 μM) (as a positive control), the drug will be added for 24 hrs post-transfection and incubated at 37° C. VX770 (positive control) will be used acutely at 5 μM concentration. For experiments using compounds, multiple concentrations will be tried. Currents will be sampled at 20 kHz and filtered at 7 kHz. Traces will be acquired at a repetition interval of 10 sec.

### Cell Death Assays

A luciferase-based assay reaction will be used to assess cell viability. This assay can be used to determine the effects on cell viability with differing treatments of a test agent. The assay format results in cell lysis and generation of a luminescent signal that is proportional to the amount of ATP present. The amount of ATP is directly proportional to the number of live cells present in a test sample. Briefly, in opaque-walled multiwell plates mammalian cells will be plated at a density of 20 k/well in culture medium. Prepare control wells containing medium without cells to determine background signal. After 24 hrs. add compounds to experimental wells and incubate for another 24 hrs. Equilibrate the plate and its contents to room temperature for approximately 30 minutes. Add 100 ul of pre-equilibrated test reagent volume (i.e. CellTiter-Glo® 2.0 Reagent) to each well equal to the volume of cell culture medium present in each well. Mix the contents for 2 minutes on an orbital shaker to induce cell lysis on a plate shaker at 500-700 rpm. Record luminescence using an integration time of 0.25-1 second per well as a guideline. The brighter the luminescent signal the more live cells you have in the sample. Viability curves versus amount of compound added can be analyzed to assess the effect of a compound on the restoration of a target of interest that results in increased cell viability.

### Cell Cycle Assays

The ability of a stabilizing compound described herein to restore the function of a protein such as a tumor suppressor can result in the cell persisting in a particular phase of the cell cycle leading to prolonging of the cell cycle and ultimately programmed cell death. The cell cycle stage at which a population of cells exists can be determined by analyzing the DNA content and distribution of the cellular DNA using flow cytometry. The assays described in Gray et al., “Cell cycle analysis using flow cytometry” International Journal of Radiation Biology and Related Studies in Physics, Chemistry and Medicine 1986, (49:2), 237-255, can be used to determine which phase of the cell cycle a cell population is in and allow for the monitoring of cell cycle changes as populations of cells are perturbed in the presence or absence of a test article.

### Enzymatic Activity Assays

Enzymatic assays will be run on targets that are enzymes such as phenylalanine hydroxylase, (PAH). Patient derived primary cells or stable cell-lines (i.e. HEK293) expressing wild type or clinically relevant mutations of PAH (i.e. R261Q or Y414C) will be used for further study. These cells will be treated with various concentrations of compounds to quantify their restorative affect. Cells will be harvested and lysed using 3× freeze-thaw cycles in Tris-KCL (0.03 uM Tris, 0.2M KCL, pH7.2) lysis buffer containing protease inhibitors. Cell lysates will be clarified for 20 min centrifugation at 3000 rcf at 4° C. The lysates will be used for activity assays. 20 ul of lysate will be incubated with 1M phenylalanine and 1 mg/ml catalase for 5 min at room temperature in 15 mM HEPES pH 7.3 followed by 1 min incubation with 10 uM ferrous ammonium sulfate. The reaction will be initiated by addition of 75 uM BH₄ stabilized in 2 mM DTT for 60 min at 25° C. and stopped by acetic acid followed by 10 min incubation at 95° C. Total reaction volume is 100 ul. The amount of tyrosine production will be measured and quantified by HPLC. The more amount of tyrosine produced will correlate with increased amounts of the PAH enzyme produced and stabilized as a function of cell treatment with a compound.

### Immunology and Immuno-Oncology Assays (Part 1)

Assays to monitor cytokine expression and release upon cell treatment with a compound will be run. To monitor the gene expression of a cytokine it is possible to use a real time RT-PCR approach. Briefly, purify cellular RNA from cells that are both treated (experimental set) and untreated (control) with Compounds. Using at least 10⁶ cells aspirate media and wash with ice cold PBS. Aspirate PBS and add 1 ml TRizol. Scrape the plate and transfer the TRizol/cell lysate into an 1.5 ml tube. Leave at RT for 5 min. Add 250 ul of chloroform and shake tube vigorously for 15 sec. Leave at RT for 5 min and then centrifuge sample at 10 k for 5 min. The resultant mixture will have three phases; remove the top phase (aqueous) and place in another tube. Add 550 ul of isopropanol to the aqueous phase and mix gently. Let sit at RT for 5 min. Centrifuge at 14 k rpm for 30 min. Place samples on ice. Pour off isopropanol and wash pellet with 75% ethanol. Recentrifuge at 9.5K rpm for 5 min. Resuspend the pellet in 25 ul of water. The resulting RNA prep should have a 260/280 ratio of >1.8. The purified RNA can now be used to create cDNA. Briefly, prepare the following reaction tube with 5 ug total RNA, 3 ul random hexamer primers (50 ng/ul), 10 mM dNTP, and bring up to 10 ul with water. Incubate the samples at 65° C. for 5 min and then on ice for at least 1 min. For each reaction add 4 ul of 25 mM MgCl₂, 1M DTT, and RNAase inhibitor, mix briefly, and then place at room temperature for 2 min. Add 50 units of reverse transcriptase to each reaction, mix and incubate at 25° C. for 10 min. Incubate the reactions at 42° C. for 50 min, heat inactivate at 70° C. for 15 min, and then chill on ice. Add 1 μl RNase H and incubate at 37° C. for 20 min. Store the cDNA at −20° C. for use in the real-time PCR experiment.

For Real time PCR design primers specific for the cytokine gene of interest you are looking to analyze the change in expression upon compound treatment. For each gene-specific forward and reverse primer pair add 2 ul of a 5 pmol/ul stock, 0.5 ul cDNA (5 ng total), 25 ul SYBR green mix, 22.5 ul water.

Run the PCR reaction in a Real Time PCR machine with the following extension times:
1. 50° C. 2 min, 1 cycle
2. 95° C. 10 min, 1 cycle
3. 95° C. 15 s->60° C. 30 s->72° C. 30 s, 40 cycles
4. 72° C. 10 min, 1 cycle
   After the PCR is finished perform a dissociation curve analysis comparing the compound treated samples to the untreated control set. A decrease of the cycle time for amplification of a particular cytokine gene under an experimental condition (compound treatment) suggests that restoration of a target of interest has led to an increase in the gene expression of a particular cytokine.

In addition to looking at cytokine expression at the transcriptional level, it is possible to analyze cytokine protein expression levels that are either secreted or produced internally in cells that are treated with varying amounts of compounds. The use of cytokine arrays has the advantage of looking at multiple cytokines at once. Briefly, seed plates and transfer media to low-serum medium (<0.2% calf serum). Treat cells with varying amounts of compounds (experimental). After 24 hrs. Collect the conditioned media. Spin at 1000 g at 4° C. for 10 min. Remove supernatant and freeze until use. Use protein concentration of cell lysate to normalize the protein amounts for the array. The cytokine array procedure is based on the sandwich ELISA technique. Commercially available membranes with immobilized antibodies to the cytokines of interest will be used. Block the membranes with bovine serum albumin for 30 min at room temperature. Incubate the membrane with sample conditioned media at room temperature for 1-2 hr. Wash membranes with TBS/Tween-20. Incubate membranes with biotin-labeled secondary antibodies at room temperature for 1-2 hours. Wash membrane with TBS/Tween-20. Incubate membranes with Horseradish peroxidate-streptavidn (HRP) at room temperature for 1 hr. Wash membranes, add HRP substrate, and visualize signal. Wells that light up are indicative of the presence of a particular cytokine secreted into the conditioned media. Comparing the signals between the test sample and the controls will allow determination of cytokine production in response to compound treatment.

### Immunology and Immuno-Oncology Assays (Part 2)

In vitro assays to analyze the effect of compounds on Tcell function will be run. For example a luciferase based assay to determine T cell proliferation in response to compound treatment will be run that is similar to the viability assay described above in the Cell Death Assays. Briefly human primary blood mononuclear cells will be seeded and treated with varying concentrations of compounds. The population of cells will then be stimulated with anti-CD28 and anti-CD03 antibodies (10 ug/ml) and the cell proliferation measured 2-day and 5-days post treatment. Cell proliferation will be measured using the amount of ATP as a surrogate for live cell proliferation (i.e. CellTiter-Glo® 2.0 Reagent). Differences in cell number between compound treated samples and untreated samples will be assessed for restoration of target function and their subsequent effect on Tcell proliferation.

### Ub-Rho Cleavage Assay

All fluorescence measurements were performed on a Molecular Devices FlexStation3 with excitation at 480 nm and emission 540 nm, PMT Gain: Medium, Flash Number: 10. The assay was performed in OptiPlate-384, White Opaque 384-well Microplate (Perkin Elmer). Assay buffer for all measurements was 50 mM HEPES, 100 mM NaCl, 0.5 mM EDTA, 1 mM TCEP, 0.1 mg/ml BSA, 0.01% Tween-20, pH 7.8. Recombinant USP 7 and Ub-Rho (R&D Systems; U-555-050) were diluted to 0.6 nM and 300 nM respectively in assay buffer to yield 2× final concentration. Serial dilutions were made with a Mosquito HTS (SPT Labtech) nanoliter liquid handler. 200 nL of each compound in duplicate or DMSO control were transferred to the assay plate. The first two columns served as positive controls. 9.8 μl per well of USP7 working solution was added to the assay plate. Compound+Protein was incubated for 25 min at 25° C. 10 uL of Ub-Rho substrate was added per well and incubated for an additional 15 min at 25° C. Fluorescence was then measured. Percent response relative to DMSO controls was calculated in GraphPad or Scinamic, and the data was fitted to a non-linear regression to determine IC₅₀ values.

DUB stock solutions were diluted in reaction buffer (50 mm Tris pH 7.6, 0.5 mm EDTA, 5 mm DTT, 0.1% (w/v) BSA) to a concentration of 2.5 nM for UCHL1 or 0.025 nM for UCHL3. Stock solutions of Ub-Rhodamine 110 (U-555, Boston Biochem, Cambridge, MA, USA); 125 nM for UCHL1 assay, and 250 nM for UCHL3 assay) were prepared in the same buffer. A 10 mM stock solution was made for each inhibitor in DMSO, then a dilution of 600 μM in reaction buffer was made followed eight by 1:1 serial dilutions. To each well was added 20 μL of DUB stock solution and 10 μL of inhibitor solutions for nine final inhibitor concentrations of ranging from 0.78 μM-200 μM along with a DMSO only control well. These were allowed to incubate, while sealed, for the 3 h at room temperature. After incubation 20 μL of each Ub-Rho stock solution was added to the respective wells for each DUB to yield final concentration of 50 nM for UCHL1 or 100 nM for UCHL3, respectively. Plates were read immediately and fluorescence of cleaved Rhodamine 110 fluorophore was monitored at λₑₓ=485 nm, λₑₘ=535 nm continuously for 20 min on a Synergy Neo2 instrument (BioTek, Winooski, VT, USA) The raw data was loaded into GraphPad Prism 8 (GraphPad Software, San Diego, CA, USA; www.graphpad.com, accessed on 12 Jan. 2021) and the slope of the linear portion of the fluorescence vs. time curves was calculation for each inhibitor concentration and % activity of the enzyme was determined compared to DMSO-treated controls. The % activity was plotted as a function of inhibitor concentration and the data was fitted with non-linear regression analysis to calculate the IC₅₀ values.

### Surface Plasmon Resonance Assay

The surface plasmon resonance experiments were performed using a Cytiva (formerly GE Healthcare) Biacore 8K equipped with a Series S Sensor Chip SA. The ligands were immobilized via a biotin-modified biotin acceptor peptide. USP7 Ligands were diluted in running buffer (HBS-P+2% DMSO; 10 mM HEPES, pH 7.4, 150 mM NaCl, 1 mM DTT, 0.05% P20, 2% DMSO) to 100 μg/mL and injected at 10 l/min until a density between 500-10,000 RU was reached on flow-cell two of each channel, and flow cell one was left blank to serve as a reference surface. Both surfaces were washed until a stable baseline was achieved then 30 startup cycles to condition the surface. To collect kinetic and steady-state binding data, the small molecule analytes were prepared in three-fold dilution series in HBS-P+2% DMSO running buffer. Analytes were injected over both flow cells at a flow rate of 30 l/min at 25° C. The complex was allowed to associate for 60 seconds and dissociate for 300 seconds. Data were collected at 10 Hz. The data were fit to a simple 1:1 interaction model global data analysis within Cytiva Biacore Insight Evaluation Software.

| | | SPR | Ub |
|---|---|---|---|
| | | USP7 CD + | (Rho) |
| | | UBL 1- | USP7.1 |
| | USP7 | 5 (KD_ss) | (IC₅₀) |
| Structure | Binder | [nM] | [nM] |
| 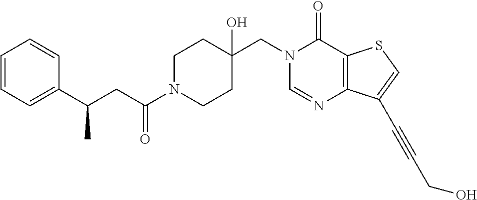 [SMILES: C[C@H](CC(=O)N1CCC(O)(Cn2cnc3c(C#CCO)csc3c2=O)CC1)c1ccccc1] | U1 | *** | *** |
| 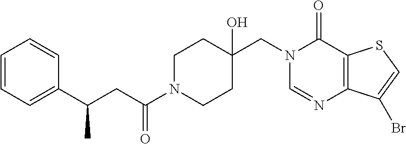 [SMILES: C[C@H](CC(=O)N1CCC(O)(Cn2cnc3c(Br)csc3c2=O)CC1)c1ccccc1] | U2 | *** | *** |
| 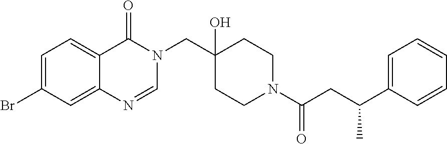 [SMILES: C[C@H](CC(=O)N1CCC(O)(Cn2cnc3cc(Br)ccc3c2=O)CC1)c1ccccc1] | U3 | ** | ** |
| 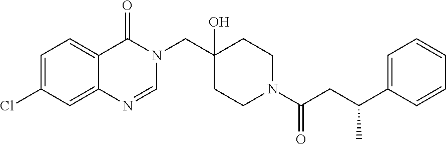 [SMILES: C[C@H](CC(=O)N1CCC(O)(Cn2cnc3cc(Cl)ccc3c2=O)CC1)c1ccccc1] | U4 | ** | ** |
| 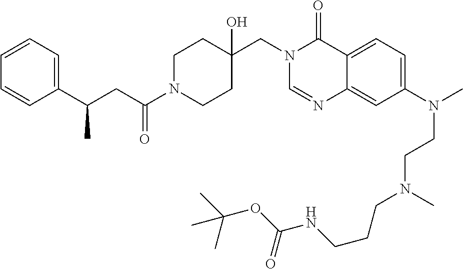 [SMILES: C[C@H](CC(=O)N1CCC(O)(Cn2cnc3cc(N(C)CCN(C)CCCNC(=O)OC(C)(C)C)ccc3c2=O)CC1)c1ccccc1] | U5 | *** | *** |
| 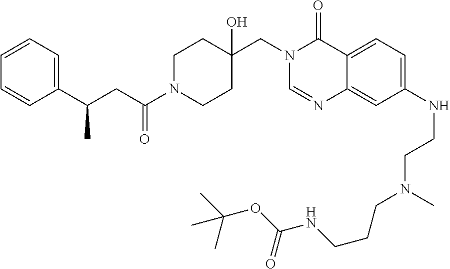 [SMILES: C[C@H](CC(=O)N1CCC(O)(Cn2cnc3cc(NCCN(C)CCCNC(=O)OC(C)(C)C)ccc3c2=O)CC1)c1ccccc1] | U6 | *** | *** |
| 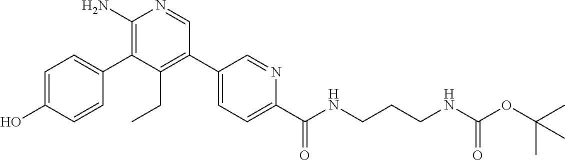 [SMILES: CCc1c(-c2ccc(C(=O)NCCCNC(=O)OC(C)(C)C)nc2)cnc(N)c1-c1ccc(O)cc1] | U7 | ** | ** |
| 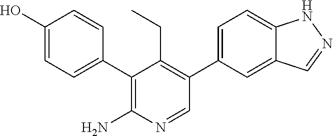 [SMILES: CCc1c(-c2ccc3[nH]ncc3c2)cnc(N)c1-c1ccc(O)cc1] | U8 | ** | ** |
| 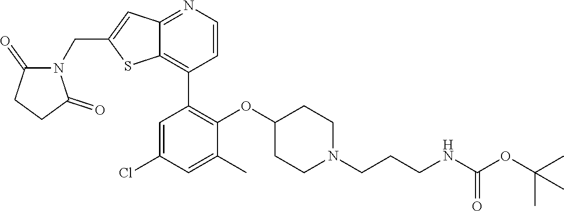 [SMILES: Cc1cc(Cl)cc(-c2ccnc3cc(CN4C(=O)CCC4=O)sc23)c1OC1CCN(CCCNC(=O)OC(C)(C)C)CC1] | U9 | *** | *** |
| 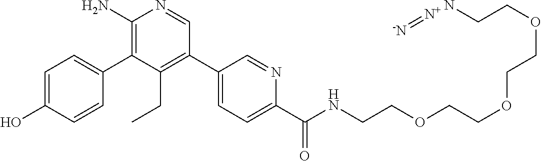 | U10 | ** | ** |
| 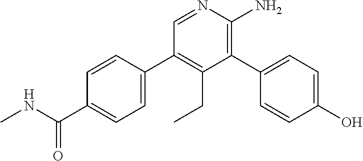 [SMILES: CCc1c(-c2ccc(C(=O)NC)cc2)cnc(N)c1-c1ccc(O)cc1] | U11 | ** | ** |
| 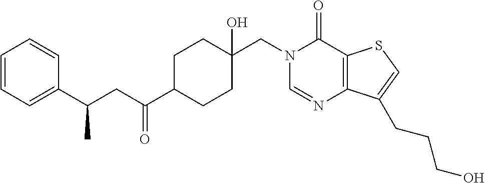 [SMILES: C[C@H](CC(=O)C1CCC(O)(Cn2cnc3c(CCCO)csc3c2=O)CC1)c1ccccc1] | U12 | ** | ** |
| 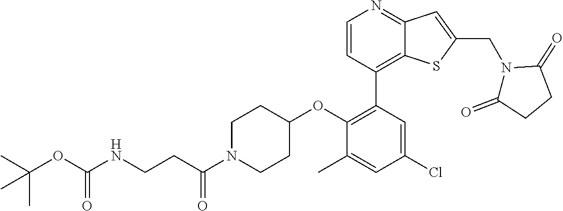 [SMILES: Cc1cc(Cl)cc(-c2ccnc3cc(CN4C(=O)CCC4=O)sc23)c1OC1CCN(C(=O)CCNC(=O)OC(C)(C)C)CC1] | U13 | ** | ** |
| 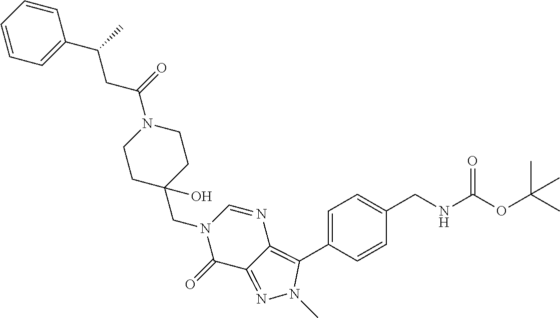 [SMILES: C[C@H](CC(=O)N1CCC(O)(Cn2cnc3c(-c4ccc(CNC(=O)OC(C)(C)C)cc4)n(C)nc3c2=O)CC1)c1ccccc1] | U14 | *** | *** |
| 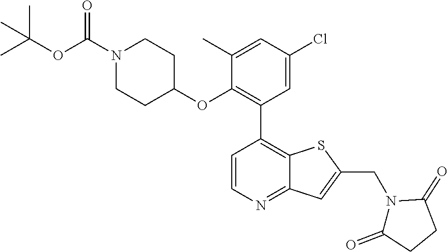 [SMILES: Cc1cc(Cl)cc(-c2ccnc3cc(CN4C(=O)CCC4=O)sc23)c1OC1CCN(C(=O)OC(C)(C)C)CC1] | U15 | ** | * |
| 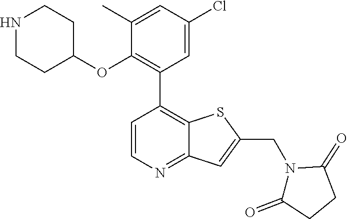 [SMILES: Cc1cc(Cl)cc(-c2ccnc3cc(CN4C(=O)CCC4=O)sc23)c1OC1CCNCC1] | U16 | *** | *** |
| 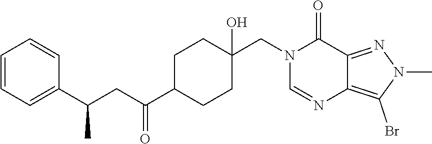 [SMILES: C[C@H](CC(=O)C1CCC(O)(Cn2cnc3c(Br)n(C)nc3c2=O)CC1)c1ccccc1] | U17 | *** | *** |
| 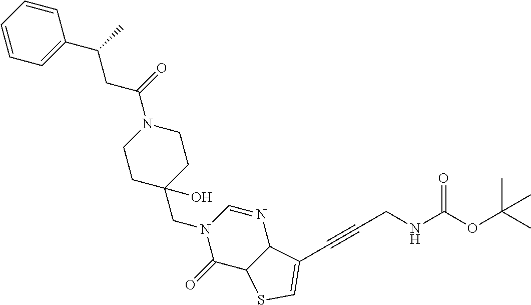 [SMILES: C[C@H](CC(=O)N1CCC(O)(CN2C=NC3C(C#CCNC(=O)OC(C)(C)C)=CSC3C2=O)CC1)c1ccccc1] | U18 | ** | *** |
| 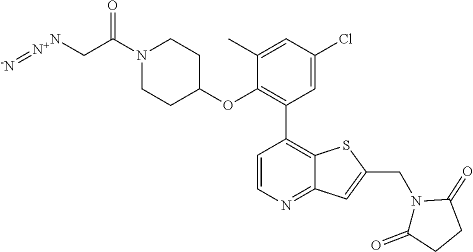 | U19 | *** | *** |
| 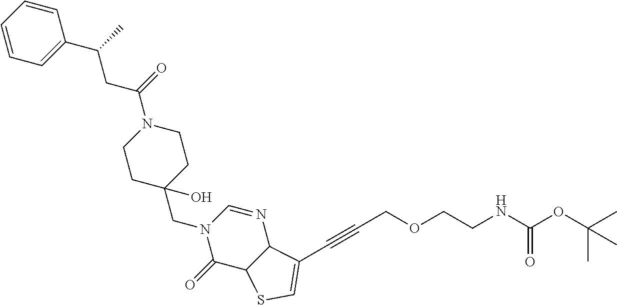 [SMILES: C[C@H](CC(=O)N1CCC(O)(CN2C=NC3C(C#CCOCCNC(=O)OC(C)(C)C)=CSC3C2=O)CC1)c1ccccc1] | U20 | ** | ** |
| 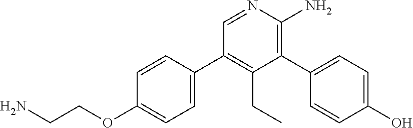 [SMILES: CCc1c(-c2ccc(OCCN)cc2)cnc(N)c1-c1ccc(O)cc1] | U21 | ** | ** |
| 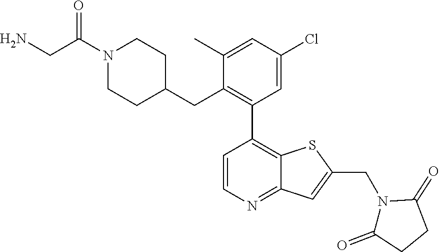 [SMILES: Cc1cc(Cl)cc(-c2ccnc3cc(CN4C(=O)CCC4=O)sc23)c1CC1CCN(C(=O)CN)CC1] | U22 | *** | *** |
| 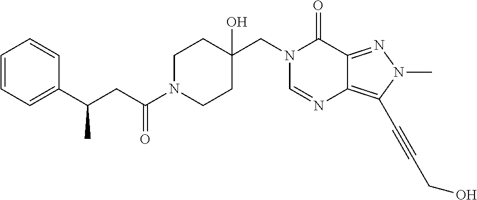 [SMILES: C[C@H](CC(=O)N1CCC(O)(Cn2cnc3c(C#CCO)n(C)nc3c2=O)CC1)c1ccccc1] | U23 | *** | *** |
| 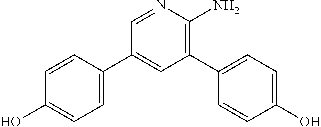 [SMILES: Nc1ncc(-c2ccc(O)cc2)cc1-c1ccc(O)cc1] | U24 | ** | ** |
| 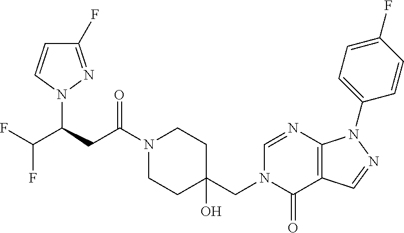 [SMILES: O=C(C[C@@H](C(F)F)n1ccc(F)n1)N1CCC(O)(Cn2cnc3c(cnn3-c3ccc(F)cc3)c2=O)CC1] | U25 | *** | *** |
| 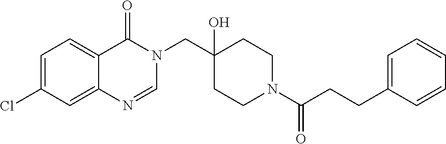 [SMILES: O=C(CCc1ccccc1)N1CCC(O)(Cn2cnc3cc(Cl)ccc3c2=O)CC1] | U26 | ** | |
| 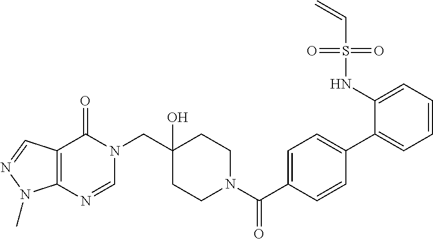 [SMILES: C=CS(=O)(=O)Nc1ccccc1-c1ccc(C(=O)N2CCC(O)(Cn3cnc4c(cnn4C)c3=O)CC2)cc1] | U27 | ** | |
| 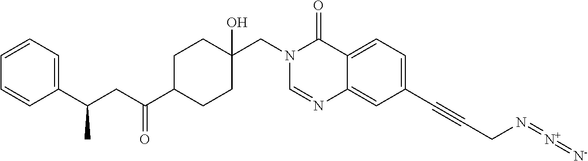 [SMILES: C[C@H](CC(=O)C1CCC(O)(Cn2cnc3cc(C#CCN=[N+]=[N-])ccc3c2=O)CC1)c1ccccc1] | U28 | *** | ** |
| 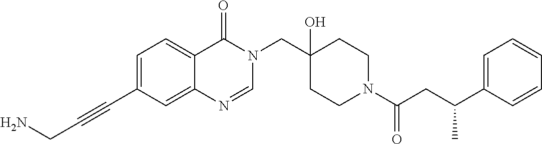 [SMILES: C[C@H](CC(=O)N1CCC(O)(Cn2cnc3cc(C#CCN)ccc3c2=O)CC1)c1ccccc1] | U29 | *** | * |
| 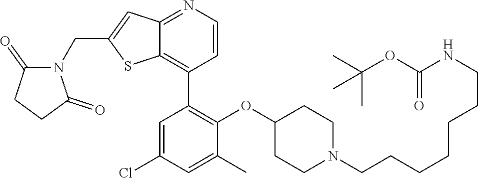 [SMILES: Cc1cc(Cl)cc(-c2ccnc3cc(CN4C(=O)CCC4=O)sc23)c1OC1CCN(CCCCCCCNC(=O)OC(C)(C)C)CC1] | U30 | *** | *** |
| 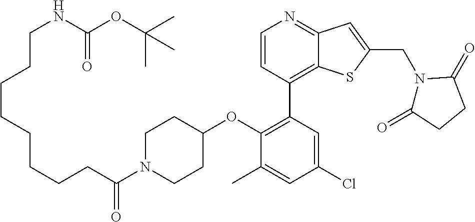 [SMILES: Cc1cc(Cl)cc(-c2ccnc3cc(CN4C(=O)CCC4=O)sc23)c1OC1CCN(C(=O)CCCCCCCCNC(=O)OC(C)(C)C)CC1] | U31 | ** | * |
| 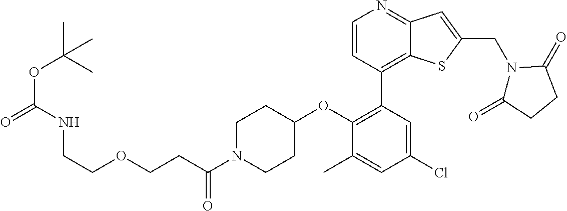 [SMILES: Cc1cc(Cl)cc(-c2ccnc3cc(CN4C(=O)CCC4=O)sc23)c1OC1CCN(C(=O)CCOCCNC(=O)OC(C)(C)C)CC1] | U32 | ** | ** |
| 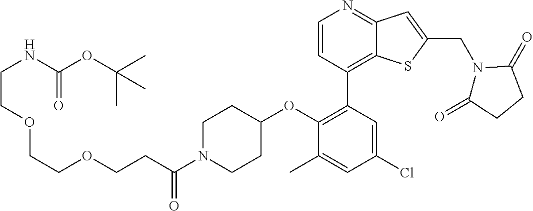 [SMILES: Cc1cc(Cl)cc(-c2ccnc3cc(CN4C(=O)CCC4=O)sc23)c1OC1CCN(C(=O)CCOCCOCCNC(=O)OC(C)(C)C)CC1] | U33 | ** | ** |
| 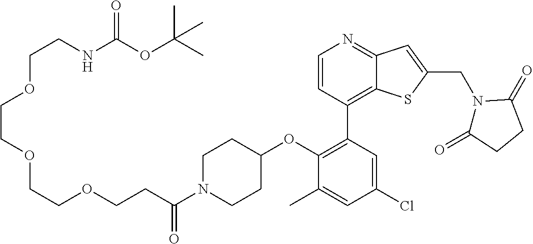 [SMILES: Cc1cc(Cl)cc(-c2ccnc3cc(CN4C(=O)CCC4=O)sc23)c1OC1CCN(C(=O)CCOCCOCCOCCNC(=O)OC(C)(C)C)CC1] | U34 | ** | ** |
| 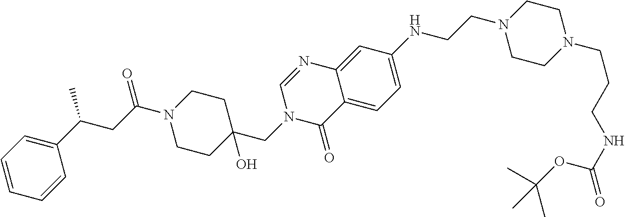 [SMILES: C[C@H](CC(=O)N1CCC(O)(Cn2cnc3cc(NCCN4CCN(CCCNC(=O)OC(C)(C)C)CC4)ccc3c2=O)CC1)c1ccccc1] | U35 | *** | *** |
| 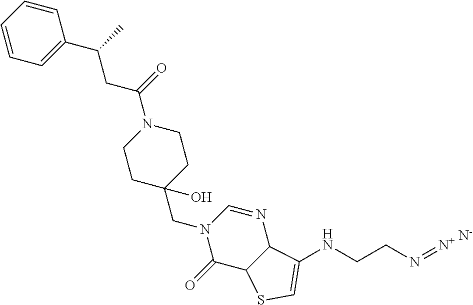 | U36 | ** | *** |
| 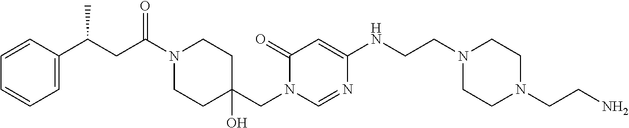 [SMILES: C[C@H](CC(=O)N1CCC(O)(Cn2cnc(NCCN3CCN(CCN)CC3)cc2=O)CC1)c1ccccc1] | U37 | *** | *** |
| 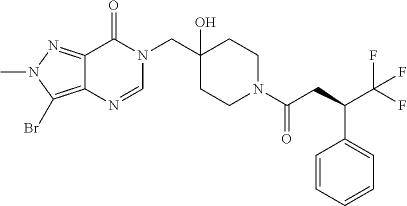 [SMILES: Cn1nc2c(=O)n(CC3(O)CCN(C(=O)C[C@H](c4ccccc4)C(F)(F)F)CC3)cnc2c1Br] | U38 | ** | ** |
| 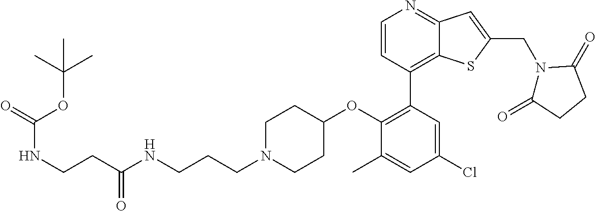 [SMILES: Cc1cc(Cl)cc(-c2ccnc3cc(CN4C(=O)CCC4=O)sc23)c1OC1CCN(CCCNC(=O)CCNC(=O)OC(C)(C)C)CC1] | U39 | *** | *** |
| 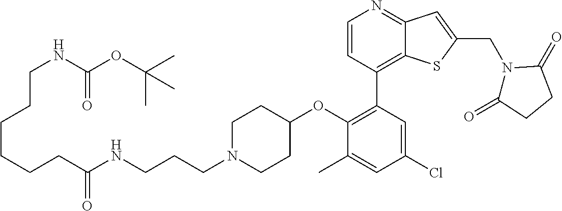 [SMILES: Cc1cc(Cl)cc(-c2ccnc3cc(CN4C(=O)CCC4=O)sc23)c1OC1CCN(CCCNC(=O)CCCCCCNC(=O)OC(C)(C)C)CC1] | U40 | *** | *** |
| 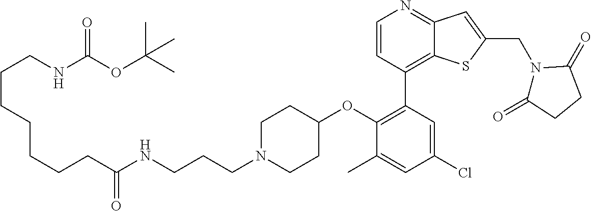 [SMILES: Cc1cc(Cl)cc(-c2ccnc3cc(CN4C(=O)CCC4=O)sc23)c1OC1CCN(CCCNC(=O)CCCCCCCNC(=O)OC(C)(C)C)CC1] | U41 | *** | *** |
| 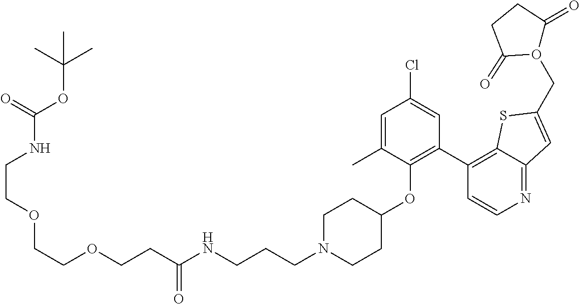 | U42 | *** | *** |
| 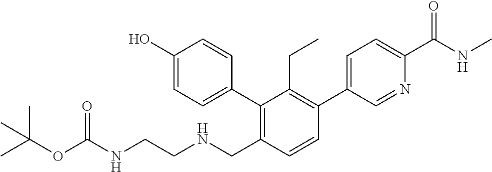 [SMILES: CCc1c(-c2ccc(C(=O)NC)nc2)ccc(CNCCNC(=O)OC(C)(C)C)c1-c1ccc(O)cc1] | U43 | ** | ** |
| 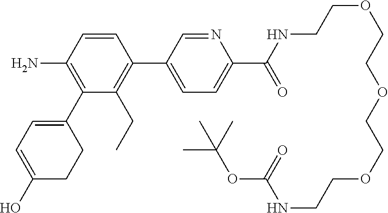 [SMILES: CCc1c(-c2ccc(C(=O)NCCOCCOCCOCCNC(=O)OC(C)(C)C)nc2)ccc(N)c1C1=CC=C(O)CC1] | U44 | ** | ** |
| 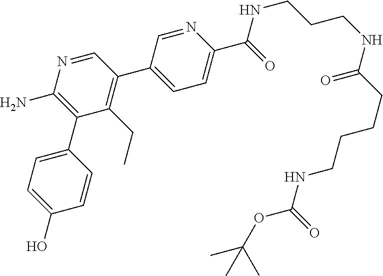 [SMILES: CCc1c(-c2ccc(C(=O)NCCCNC(=O)CCCCNC(=O)OC(C)(C)C)nc2)cnc(N)c1-c1ccc(O)cc1] | U45 | ** | ** |
| 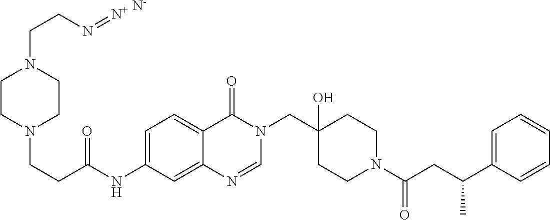 | U46 | *** | *** |
| 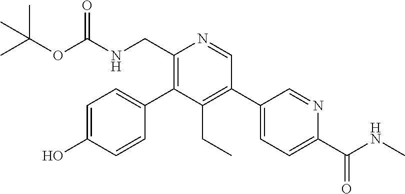 [SMILES: CCc1c(-c2ccc(C(=O)NC)nc2)cnc(CNC(=O)OC(C)(C)C)c1-c1ccc(O)cc1] | U47 | ** | ** |
| 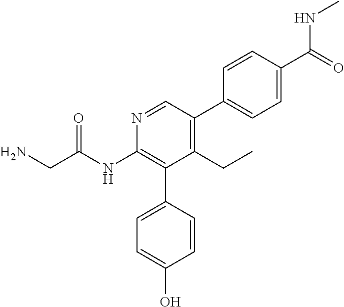 [SMILES: CCc1c(-c2ccc(C(=O)NC)cc2)cnc(NC(=O)CN)c1-c1ccc(O)cc1] | U48 | ** | * |
| 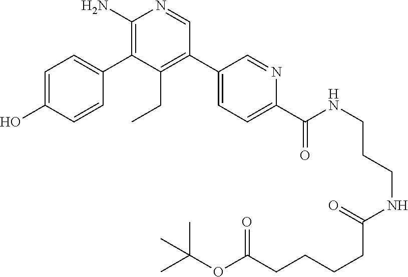 [SMILES: CCc1c(-c2ccc(C(=O)NCCCNC(=O)CCCCC(=O)OC(C)(C)C)nc2)cnc(N)c1-c1ccc(O)cc1] | U49 | ** | ** |
| 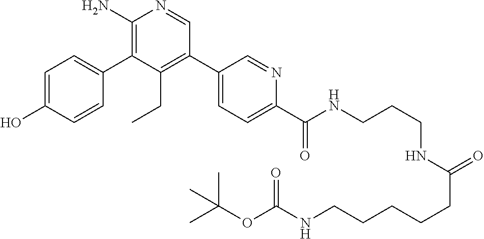 [SMILES: CCc1c(-c2ccc(C(=O)NCCCNC(=O)CCCCCNC(=O)OC(C)(C)C)nc2)cnc(N)c1-c1ccc(O)cc1] | U50 | ** | ** |
| 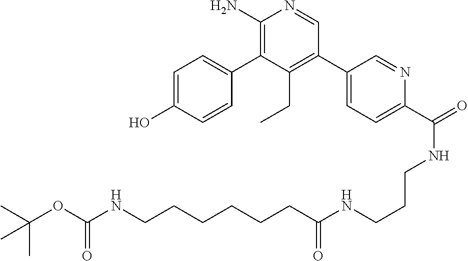 [SMILES: CCc1c(-c2ccc(C(=O)NCCCNC(=O)CCCCCCNC(=O)OC(C)(C)C)nc2)cnc(N)c1-c1ccc(O)cc1] | U51 | ** | * |
| 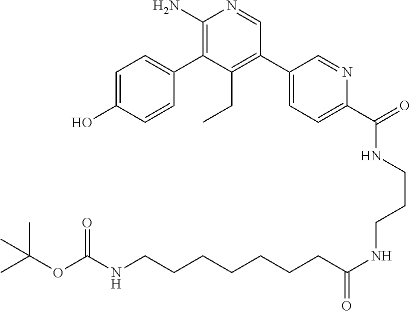 [SMILES: CCc1c(-c2ccc(C(=O)NCCCNC(=O)CCCCCCCNC(=O)OC(C)(C)C)nc2)cnc(N)c1-c1ccc(O)cc1] | U52 | ** | * |
| 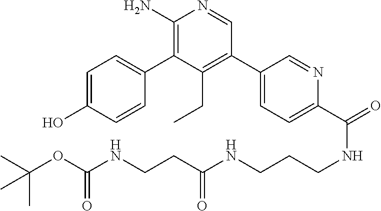 [SMILES: CCc1c(-c2ccc(C(=O)NCCCNC(=O)CCNC(=O)OC(C)(C)C)nc2)cnc(N)c1-c1ccc(O)cc1] | U53 | ** | ** |
| 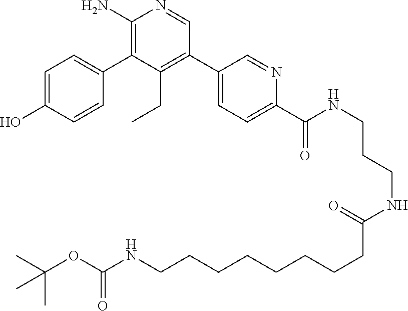 [SMILES: CCc1c(-c2ccc(C(=O)NCCCNC(=O)CCCCCCCCNC(=O)OC(C)(C)C)nc2)cnc(N)c1-c1ccc(O)cc1] | U54 | ** | * |
| 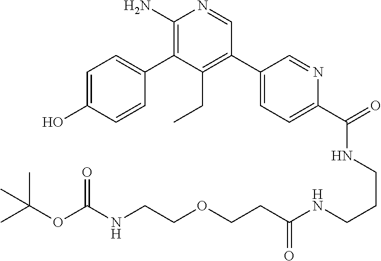 [SMILES: CCc1c(-c2ccc(C(=O)NCCCNC(=O)CCOCCNC(=O)OC(C)(C)C)nc2)cnc(N)c1-c1ccc(O)cc1] | U55 | ** | ** |
| 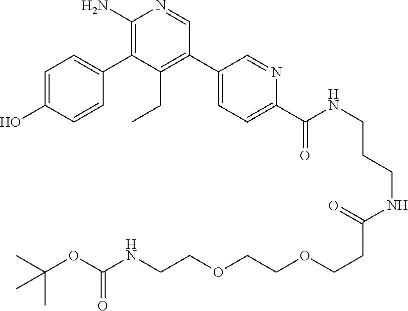 [SMILES: CCc1c(-c2ccc(C(=O)NCCCNC(=O)CCOCCOCCNC(=O)OC(C)(C)C)nc2)cnc(N)c1-c1ccc(O)cc1] | U56 | ** | ** |
| 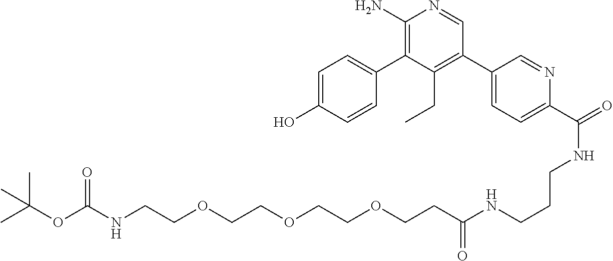 [SMILES: CCc1c(-c2ccc(C(=O)NCCCNC(=O)CCOCCOCCOCCNC(=O)OC(C)(C)C)nc2)cnc(N)c1-c1ccc(O)cc1] | U57 | ** | ** |
| 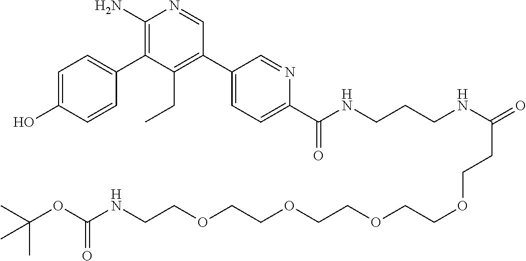 [SMILES: CCc1c(-c2ccc(C(=O)NCCCNC(=O)CCOCCOCCOCCOCCNC(=O)OC(C)(C)C)nc2)cnc(N)c1-c1ccc(O)cc1] | U58 | ** | ** |
| 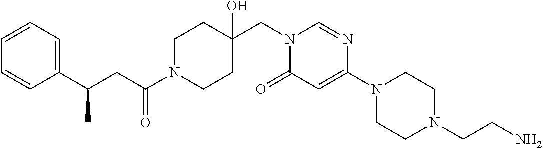 [SMILES: C[C@H](CC(=O)N1CCC(O)(Cn2cnc(N3CCN(CCN)CC3)cc2=O)CC1)c1ccccc1] | U59 | *** | ** |
| 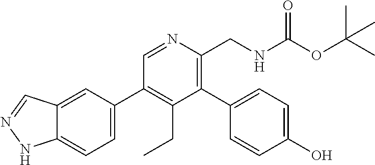 [SMILES: CCc1c(-c2ccc3[nH]ncc3c2)cnc(CNC(=O)OC(C)(C)C)c1-c1ccc(O)cc1] | U60 | ** | ** |
| 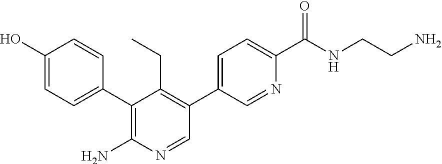 [SMILES: CCc1c(-c2ccc(C(=O)NCCN)nc2)cnc(N)c1-c1ccc(O)cc1] | U61 | *** | *** |
| 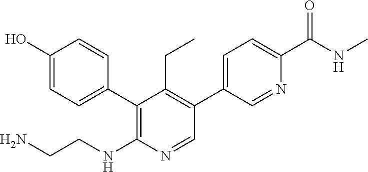 [SMILES: CCc1c(-c2ccc(C(=O)NC)nc2)cnc(NCCN)c1-c1ccc(O)cc1] | U62 | *** | ** |
| 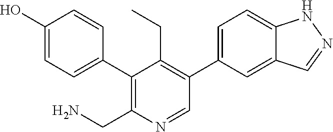 [SMILES: CCc1c(-c2ccc3[nH]ncc3c2)cnc(CN)c1-c1ccc(O)cc1] | U63 | ** | ** |
| 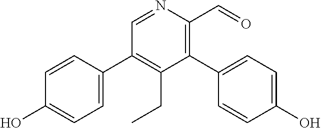 [SMILES: CCc1c(-c2ccc(O)cc2)cnc(C=O)c1-c1ccc(O)cc1] | U64 | ** | ** |
| 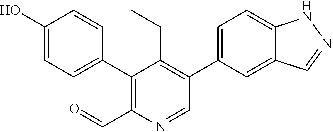 [SMILES: CCc1c(-c2ccc3[nH]ncc3c2)cnc(C=O)c1-c1ccc(O)cc1] | U65 | ** | ** |
| 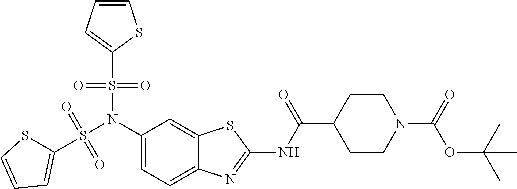 [SMILES: CC(C)(C)OC(=O)N1CCC(C(=O)Nc2nc3ccc(N(S(=O)(=O)c4cccs4)S(=O)(=O)c4cccs4)cc3s2)CC1] | U66 | ** | * |
| 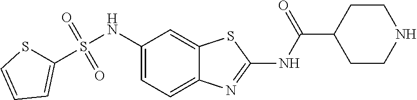 [SMILES: O=C(Nc1nc2ccc(NS(=O)(=O)c3cccs3)cc2s1)C1CCNCC1] | U67 | ** | * |
| 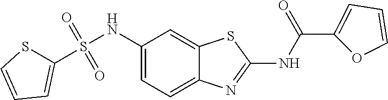 [SMILES: O=C(Nc1nc2ccc(NS(=O)(=O)c3cccs3)cc2s1)c1ccco1] | U68 | ** | * |
| 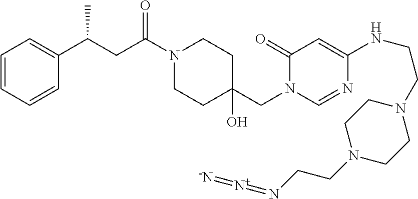 | U69 | *** | *** |
| 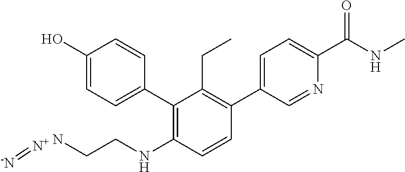 | U70 | ** | ** |
| 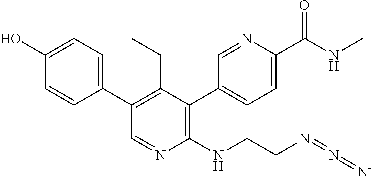 [SMILES: CCc1c(-c2ccc(O)cc2)cnc(NCCN=[N+]=[N-])c1-c1ccc(C(=O)NC)nc1] | U71 | *** | ** |
| 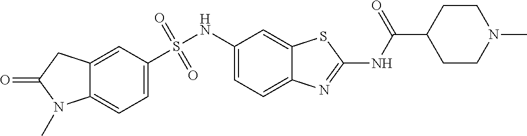 [SMILES: CN1CCC(C(=O)Nc2nc3ccc(NS(=O)(=O)c4ccc5c(c4)CC(=O)N5C)cc3s2)CC1] | U72 | * | * |
| 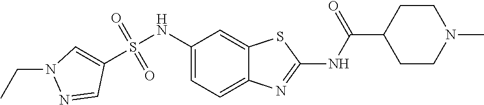 [SMILES: CCn1cc(S(=O)(=O)Nc2ccc3nc(NC(=O)C4CCN(C)CC4)sc3c2)cn1] | U73 | * | ** |
| 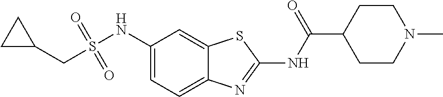 [SMILES: CN1CCC(C(=O)Nc2nc3ccc(NS(=O)(=O)CC4CC4)cc3s2)CC1] | U74 | ** | * |
| 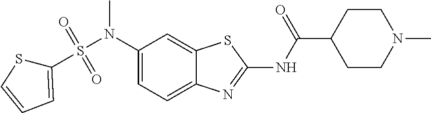 [SMILES: CN1CCC(C(=O)Nc2nc3ccc(N(C)S(=O)(=O)c4cccs4)cc3s2)CC1] | U75 | * | * |
| 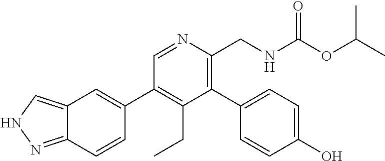 [SMILES: CCc1c(-c2ccc3n[nH]cc3c2)cnc(CNC(=O)OC(C)C)c1-c1ccc(O)cc1] | U76 | ** | ** |
| 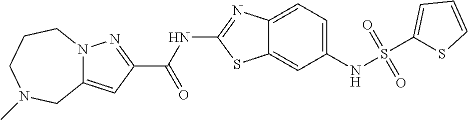 [SMILES: CN1CCCn2nc(C(=O)Nc3nc4ccc(NS(=O)(=O)c5cccs5)cc4s3)cc2C1] | U77 | ** | * |
| 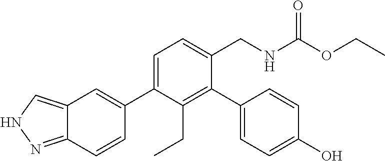 [SMILES: CCOC(=O)NCc1ccc(-c2ccc3n[nH]cc3c2)c(CC)c1-c1ccc(O)cc1] | U78 | ** | ** |
| 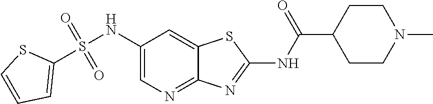 [SMILES: CN1CCC(C(=O)Nc2nc3ncc(NS(=O)(=O)c4cccs4)cc3s2)CC1] | U79 | * | * |
| 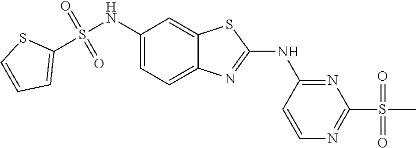 [SMILES: CS(=O)(=O)c1nccc(Nc2nc3ccc(NS(=O)(=O)c4cccs4)cc3s2)n1] | U80 | ** | * |
| 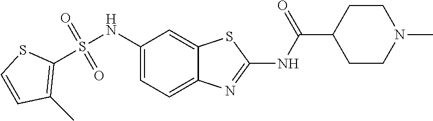 [SMILES: Cc1ccsc1S(=O)(=O)Nc1ccc2nc(NC(=O)C3CCN(C)CC3)sc2c1] | U81 | ** | * |
| 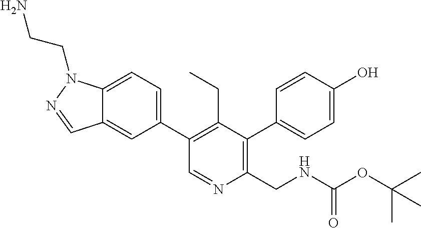 [SMILES: CCc1c(-c2ccc3c(cnn3CCN)c2)cnc(CNC(=O)OC(C)(C)C)c1-c1ccc(O)cc1] | U82 | ** | ** |
| 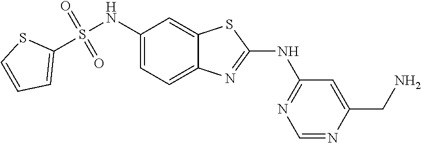 [SMILES: NCc1cc(Nc2nc3ccc(NS(=O)(=O)c4cccs4)cc3s2)ncn1] | U83 | ** | * |
| 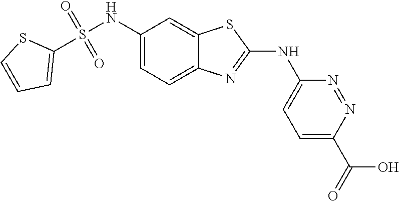 [SMILES: O=C(O)c1ccc(Nc2nc3ccc(NS(=O)(=O)c4cccs4)cc3s2)nn1] | U84 | ** | * |
| 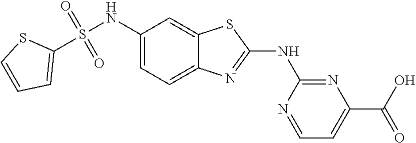 [SMILES: O=C(O)c1ccnc(Nc2nc3ccc(NS(=O)(=O)c4cccs4)cc3s2)n1] | U85 | * | * |
| 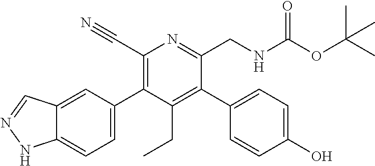 [SMILES: CCc1c(-c2ccc3[nH]ncc3c2)c(C#N)nc(CNC(=O)OC(C)(C)C)c1-c1ccc(O)cc1] | U86 | ** | ** |
| 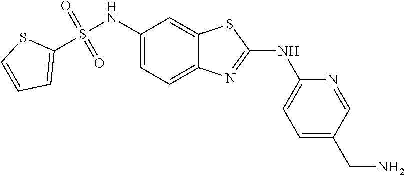 [SMILES: NCc1ccc(Nc2nc3ccc(NS(=O)(=O)c4cccs4)cc3s2)nc1] | U87 | ** | * |
| 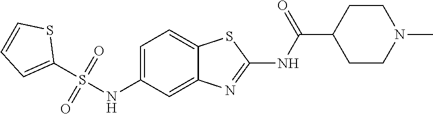 [SMILES: CN1CCC(C(=O)Nc2nc3cc(NS(=O)(=O)c4cccs4)ccc3s2)CC1] | U88 | * | * |
| 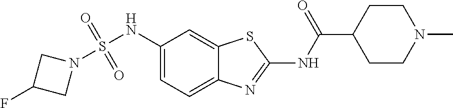 [SMILES: CN1CCC(C(=O)Nc2nc3ccc(NS(=O)(=O)N4CC(F)C4)cc3s2)CC1] | U89 | * | * |
| 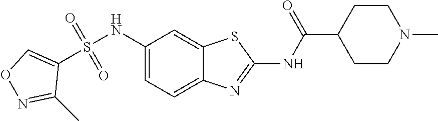 [SMILES: Cc1nocc1S(=O)(=O)Nc1ccc2nc(NC(=O)C3CCN(C)CC3)sc2c1] | U90 | ** | * |
| 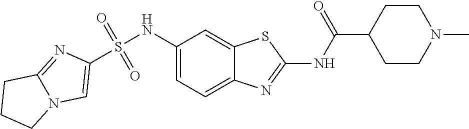 [SMILES: CN1CCC(C(=O)Nc2nc3ccc(NS(=O)(=O)c4cn5c(n4)CCC5)cc3s2)CC1] | U91 | ** | * |
| 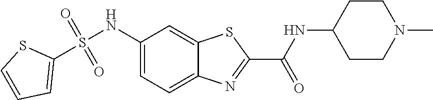 [SMILES: CN1CCC(NC(=O)c2nc3ccc(NS(=O)(=O)c4cccs4)cc3s2)CC1] | U92 | * | * |
| 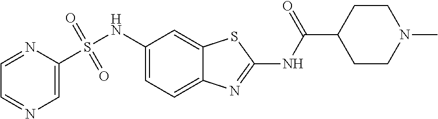 [SMILES: CN1CCC(C(=O)Nc2nc3ccc(NS(=O)(=O)c4cnccn4)cc3s2)CC1] | U93 | ** | * |
| 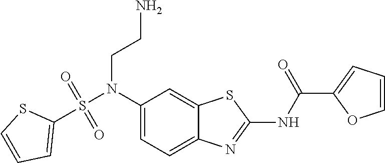 [SMILES: NCCN(c1ccc2nc(NC(=O)c3ccco3)sc2c1)S(=O)(=O)c1cccs1] | U94 | ** | * |
| 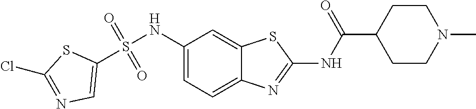 [SMILES: CN1CCC(C(=O)Nc2nc3ccc(NS(=O)(=O)c4cnc(Cl)s4)cc3s2)CC1] | U95 | * | * |
| 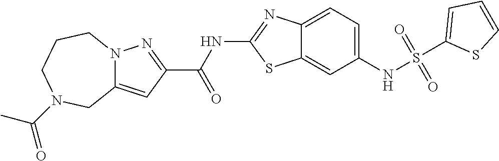 [SMILES: CC(=O)N1CCCn2nc(C(=O)Nc3nc4ccc(NS(=O)(=O)c5cccs5)cc4s3)cc2C1] | U96 | ** | * |
| 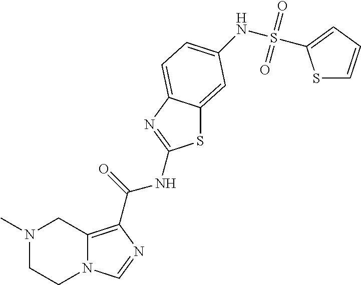 [SMILES: CN1CCn2cnc(C(=O)Nc3nc4ccc(NS(=O)(=O)c5cccs5)cc4s3)c2C1] | U97 | ** | * |
| 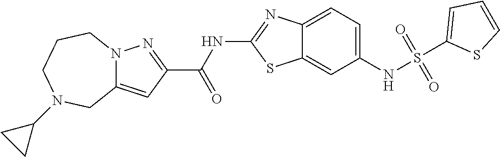 [SMILES: O=C(Nc1nc2ccc(NS(=O)(=O)c3cccs3)cc2s1)c1cc2n(n1)CCCN(C1CC1)C2] | U98 | ** | ** |
| 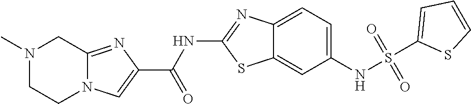 [SMILES: CN1CCn2cc(C(=O)Nc3nc4ccc(NS(=O)(=O)c5cccs5)cc4s3)nc2C1] | U99 | ** | * |
| 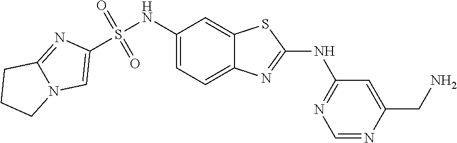 [SMILES: NCc1cc(Nc2nc3ccc(NS(=O)(=O)c4cn5c(n4)CCC5)cc3s2)ncn1] | U100 | ** | * |
| 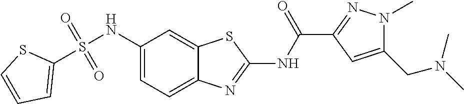 [SMILES: CN(C)Cc1cc(C(=O)Nc2nc3ccc(NS(=O)(=O)c4cccs4)cc3s2)nn1C] | U101 | ** | * |
| 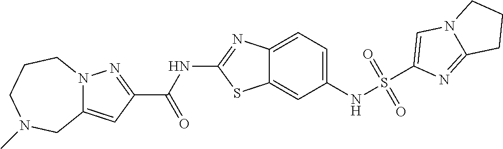 [SMILES: CN1CCCn2nc(C(=O)Nc3nc4ccc(NS(=O)(=O)c5cn6c(n5)CCC6)cc4s3)cc2C1] | U102 | ** | * |
| 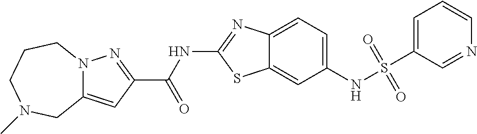 [SMILES: CN1CCCn2nc(C(=O)Nc3nc4ccc(NS(=O)(=O)c5cccnc5)cc4s3)cc2C1] | U103 | ** | * |
| 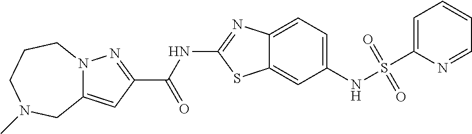 [SMILES: CN1CCCn2nc(C(=O)Nc3nc4ccc(NS(=O)(=O)c5ccccn5)cc4s3)cc2C1] | U104 | ** | * |
| 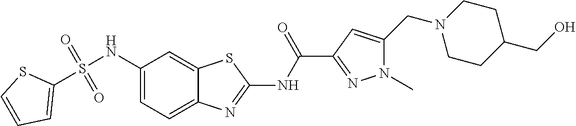 [SMILES: Cn1nc(C(=O)Nc2nc3ccc(NS(=O)(=O)c4cccs4)cc3s2)cc1CN1CCC(CO)CC1] | U105 | ** | * |
| 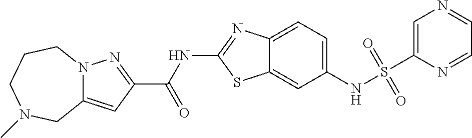 [SMILES: CN1CCCn2nc(C(=O)Nc3nc4ccc(NS(=O)(=O)c5cnccn5)cc4s3)cc2C1] | U106 | * | * |
| 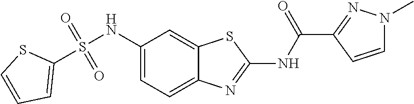 [SMILES: Cn1ccc(C(=O)Nc2nc3ccc(NS(=O)(=O)c4cccs4)cc3s2)n1] | U107 | ** | * |
| 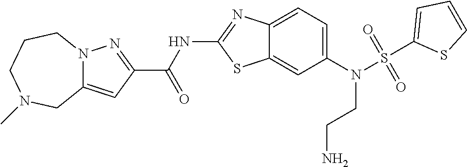 [SMILES: CN1CCCn2nc(C(=O)Nc3nc4ccc(N(CCN)S(=O)(=O)c5cccs5)cc4s3)cc2C1] | U108 | * | * |
| 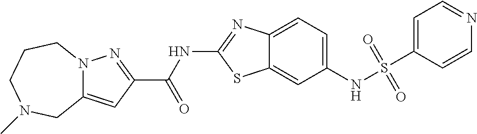 [SMILES: CN1CCCn2nc(C(=O)Nc3nc4ccc(NS(=O)(=O)c5ccncc5)cc4s3)cc2C1] | U109 | * | * |
| 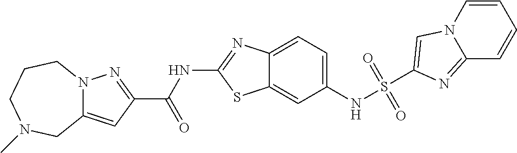 [SMILES: CN1CCCn2nc(C(=O)Nc3nc4ccc(NS(=O)(=O)c5cn6ccccc6n5)cc4s3)cc2C1] | U110 | ** | * |
| 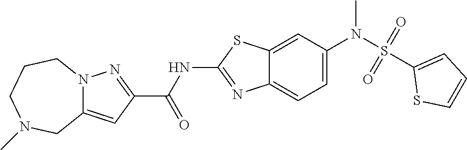 [SMILES: CN1CCCn2nc(C(=O)Nc3nc4ccc(N(C)S(=O)(=O)c5cccs5)cc4s3)cc2C1] | U111 | * | * |
| 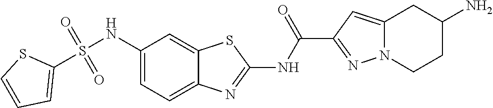 [SMILES: NC1CCn2nc(C(=O)Nc3nc4ccc(NS(=O)(=O)c5cccs5)cc4s3)cc2C1] | U112 | ** | * |
| 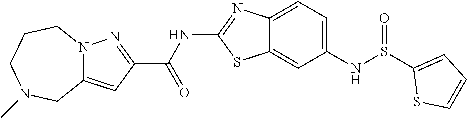 [SMILES: CN1CCCn2nc(C(=O)Nc3nc4ccc(NS(=O)c5cccs5)cc4s3)cc2C1] | U113 | ** | * |
| 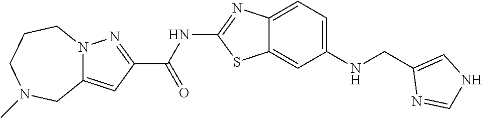 [SMILES: CN1CCCn2nc(C(=O)Nc3nc4ccc(NCc5c[nH]cn5)cc4s3)cc2C1] | U114 | * | * |
| 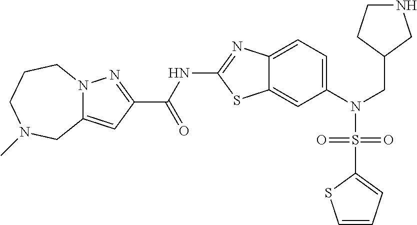 [SMILES: CN1CCCn2nc(C(=O)Nc3nc4ccc(N(CC5CCNC5)S(=O)(=O)c5cccs5)cc4s3)cc2C1] | U115 | ** | * |
| 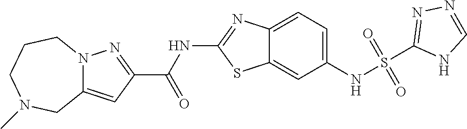 [SMILES: CN1CCCn2nc(C(=O)Nc3nc4ccc(NS(=O)(=O)c5nnc[nH]5)cc4s3)cc2C1] | U116 | ** | * |
| 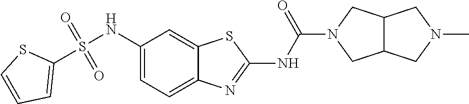 [SMILES: CN1CC2CN(C(=O)Nc3nc4ccc(NS(=O)(=O)c5cccs5)cc4s3)CC2C1] | U117 | ** | * |
| 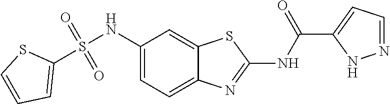 [SMILES: O=C(Nc1nc2ccc(NS(=O)(=O)c3cccs3)cc2s1)c1ccn[nH]1] | U118 | ** | * |
| 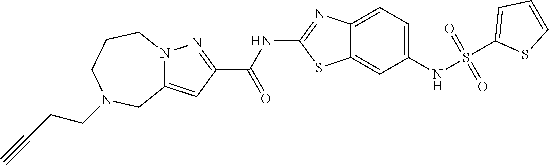 [SMILES: C#CCCN1CCCn2nc(C(=O)Nc3nc4ccc(NS(=O)(=O)c5cccs5)cc4s3)cc2C1] | U119 | ** | * |
| 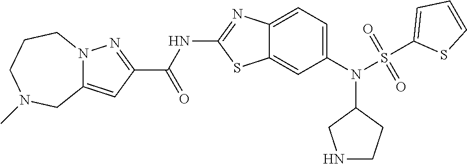 [SMILES: CN1CCCn2nc(C(=O)Nc3nc4ccc(N(C5CCNC5)S(=O)(=O)c5cccs5)cc4s3)cc2C1] | U120 | ** | * |
| 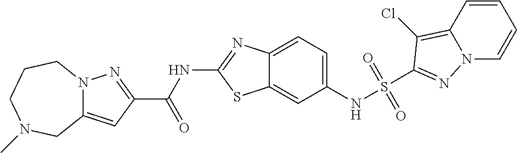 [SMILES: CN1CCCn2nc(C(=O)Nc3nc4ccc(NS(=O)(=O)c5nn6ccccc6c5Cl)cc4s3)cc2C1] | U121 | ** | * |
| 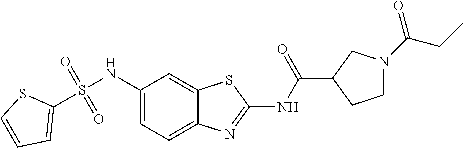 [SMILES: CCC(=O)N1CCC(C(=O)Nc2nc3ccc(NS(=O)(=O)c4cccs4)cc3s2)C1] | U122 | ** | * |
| 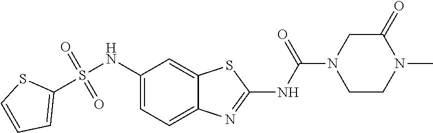 [SMILES: CN1CCN(C(=O)Nc2nc3ccc(NS(=O)(=O)c4cccs4)cc3s2)CC1=O] | U123 | * | * |
| 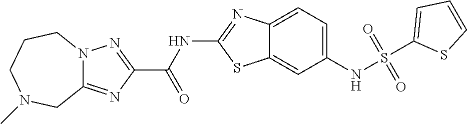 [SMILES: CN1CCCn2nc(C(=O)Nc3nc4ccc(NS(=O)(=O)c5cccs5)cc4s3)nc2C1] | U124 | * | ** |
| 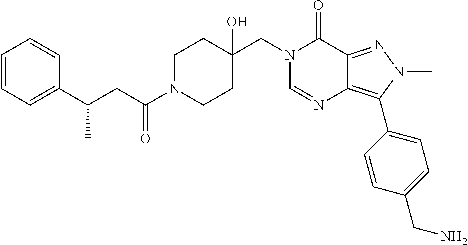 [SMILES: C[C@@H](CC(=O)N1CCC(O)(Cn2cnc3c(-c4ccc(CN)cc4)n(C)nc3c2=O)CC1)c1ccccc1] | U125 | ** | *** |
| 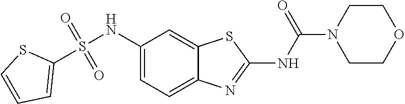 [SMILES: O=C(Nc1nc2ccc(NS(=O)(=O)c3cccs3)cc2s1)N1CCOCC1] | U126 | ** | * |
| 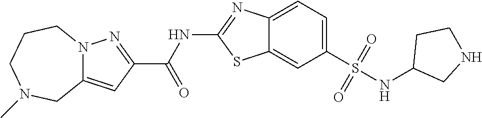 [SMILES: CN1CCCn2nc(C(=O)Nc3nc4ccc(S(=O)(=O)NC5CCNC5)cc4s3)cc2C1] | U127 | ** | * |
| 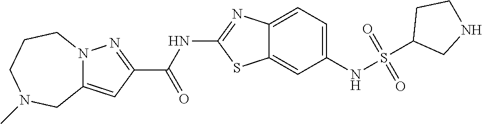 [SMILES: CN1CCCn2nc(C(=O)Nc3nc4ccc(NS(=O)(=O)C5CCNC5)cc4s3)cc2C1] | U128 | ** | * |
| 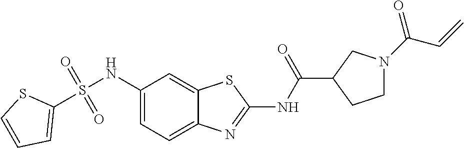 [SMILES: C=CC(=O)N1CCC(C(=O)Nc2nc3ccc(NS(=O)(=O)c4cccs4)cc3s2)C1] | U129 | ** | * |
| 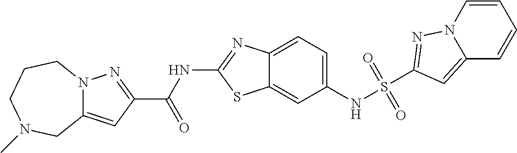 [SMILES: CN1CCCn2nc(C(=O)Nc3nc4ccc(NS(=O)(=O)c5cc6ccccn6n5)cc4s3)cc2C1] | U130 | ** | * |

* is > 100 μM; ** is <100 μM and > than 1 μM; *** is < 1 μM

| | | SPR | SPR | |
|---|---|---|---|---|
| | | USP7 | USP7 | |
| | | CD + | CD + | |
| | | UBL | UBL | Ub |
| | | 1-5 | 1-5 | (Rho) |
| | | (KD_ | (KD_ | USP7.1 |
| | USP7 | kinetic) | ss) | (IC50) |
| Structure | Binder | [nM] | [nM] | [nM] |
| 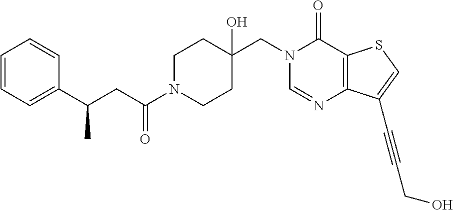 [SMILES: C[C@H](CC(=O)N1CCC(O)(Cn2cnc3c(C#CCO)csc3c2=O)CC1)c1ccccc1] | U1 | *** | *** | *** |
| 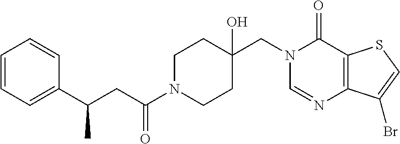 [SMILES: C[C@H](CC(=O)N1CCC(O)(Cn2cnc3c(Br)csc3c2=O)CC1)c1ccccc1] | U2 | *** | *** | *** |
| 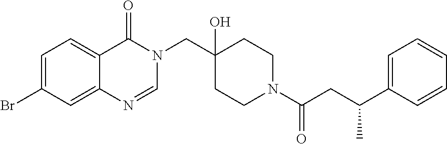 [SMILES: C[C@H](CC(=O)N1CCC(O)(Cn2cnc3cc(Br)ccc3c2=O)CC1)c1ccccc1] | U3 | ** | ** | ** |
| 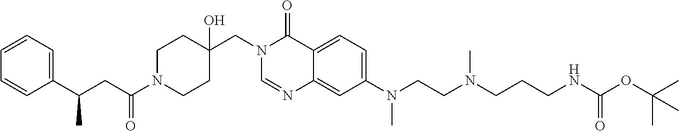 [SMILES: C[C@H](CC(=O)N1CCC(O)(Cn2cnc3cc(N(C)CCN(C)CCCNC(=O)OC(C)(C)C)ccc3c2=O)CC1)c1ccccc1] | U5 | *** | *** | *** |
| 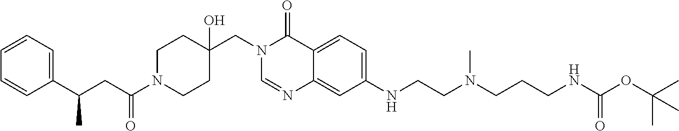 [SMILES: C[C@H](CC(=O)N1CCC(O)(Cn2cnc3cc(NCCN(C)CCCNC(=O)OC(C)(C)C)ccc3c2=O)CC1)c1ccccc1] | U6 | *** | *** | *** |
| 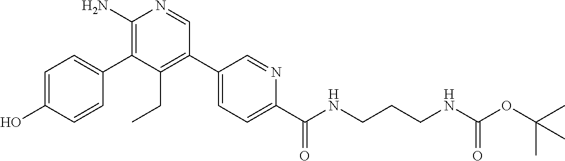 [SMILES: CCc1c(-c2ccc(C(=O)NCCCNC(=O)OC(C)(C)C)nc2)cnc(N)c1-c1ccc(O)cc1] | U7 | ** | ** | ** |
| 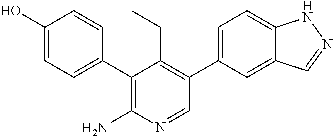 [SMILES: CCc1c(-c2ccc3[nH]ncc3c2)cnc(N)c1-c1ccc(O)cc1] | U8 | ** | ** | ** |
| 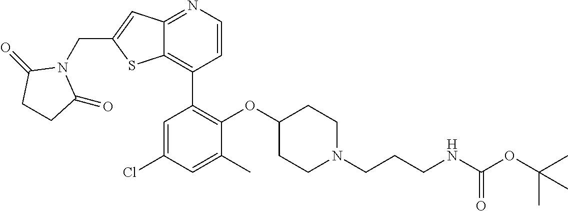 [SMILES: Cc1cc(Cl)cc(-c2ccnc3cc(CN4C(=O)CCC4=O)sc23)c1OC1CCN(CCCNC(=O)OC(C)(C)C)CC1] | U9 | *** | *** | *** |
| 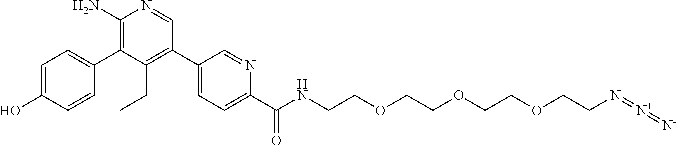 [SMILES: CCc1c(-c2ccc(C(=O)NCCOCCOCCOCCN=[N+]=[N-])nc2)cnc(N)c1-c1ccc(O)cc1] | U10 | ** | ** | ** |
| 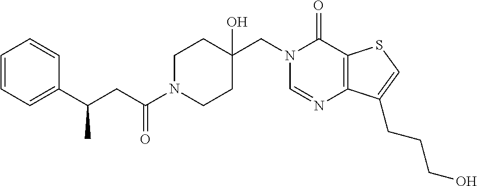 [SMILES: C[C@H](CC(=O)N1CCC(O)(Cn2cnc3c(CCCO)csc3c2=O)CC1)c1ccccc1] | U12 | ** | ** | ** |
| 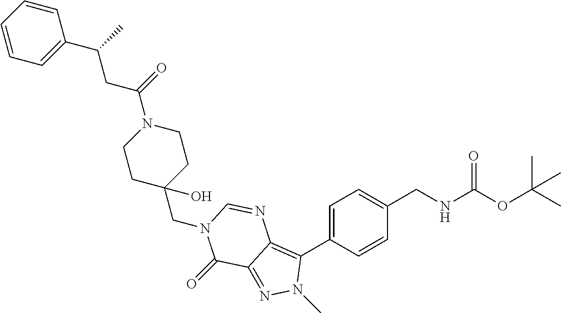 [SMILES: C[C@H](CC(=O)N1CCC(O)(Cn2cnc3c(-c4ccc(CNC(=O)OC(C)(C)C)cc4)n(C)nc3c2=O)CC1)c1ccccc1] | U14 | *** | *** | *** |
| 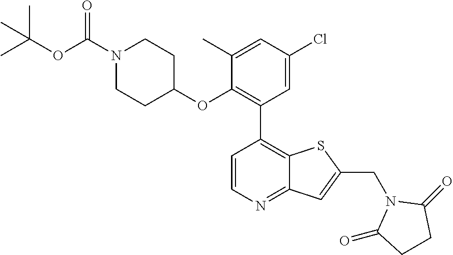 [SMILES: Cc1cc(Cl)cc(-c2ccnc3cc(CN4C(=O)CCC4=O)sc23)c1OC1CCN(C(=O)OC(C)(C)C)CC1] | U15 | ** | ** | * |
| 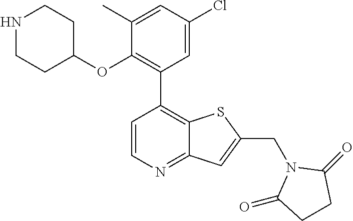 [SMILES: Cc1cc(Cl)cc(-c2ccnc3cc(CN4C(=O)CCC4=O)sc23)c1OC1CCNCC1] | U16 | *** | *** | *** |
| 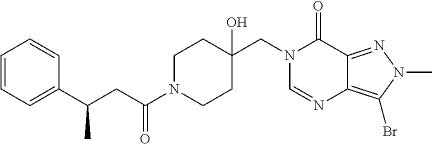 [SMILES: C[C@H](CC(=O)N1CCC(O)(Cn2cnc3c(Br)n(C)nc3c2=O)CC1)c1ccccc1] | U17 | *** | *** | *** |
| 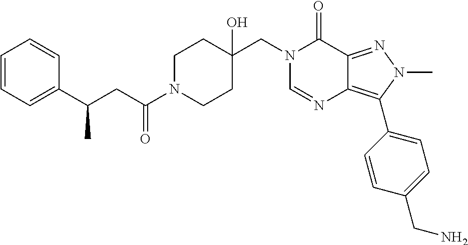 [SMILES: C[C@H](CC(=O)N1CCC(O)(Cn2cnc3c(-c4ccc(CN)cc4)n(C)nc3c2=O)CC1)c1ccccc1] | U125 | *** | | *** |
| 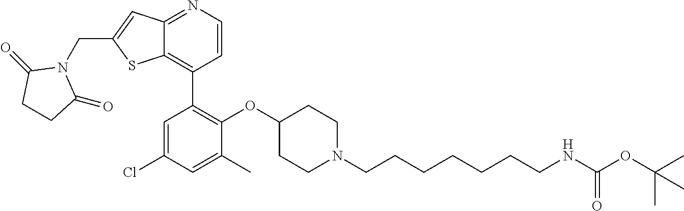 [SMILES: Cc1cc(Cl)cc(-c2ccnc3cc(CN4C(=O)CCC4=O)sc23)c1OC1CCN(CCCCCCCNC(=O)OC(C)(C)C)CC1] | U30 | *** | *** | *** |
| 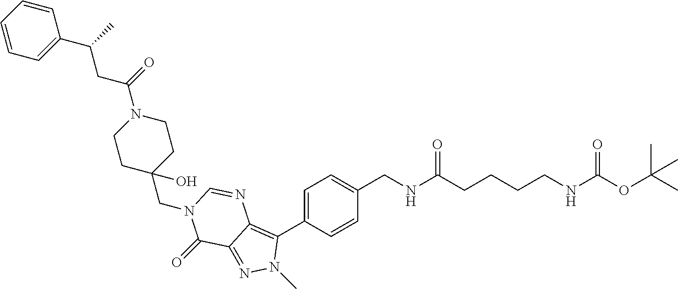 [SMILES: C[C@H](CC(=O)N1CCC(O)(Cn2cnc3c(-c4ccc(CNC(=O)CCCCNC(=O)OC(C)(C)C)cc4)n(C)nc3c2=O)CC1)c1ccccc1] | U131 | *** | | *** |
| 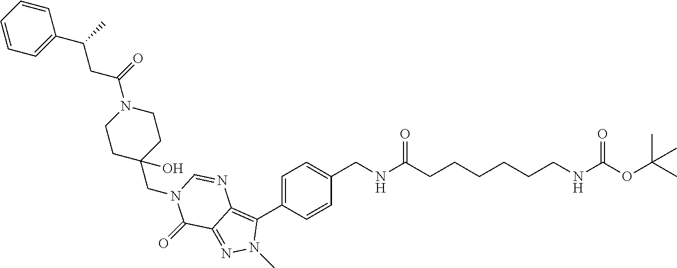 [SMILES: C[C@H](CC(=O)N1CCC(O)(Cn2cnc3c(-c4ccc(CNC(=O)CCCCCCNC(=O)OC(C)(C)C)cc4)n(C)nc3c2=O)CC1)c1ccccc1] | U132 | *** | | *** |
| 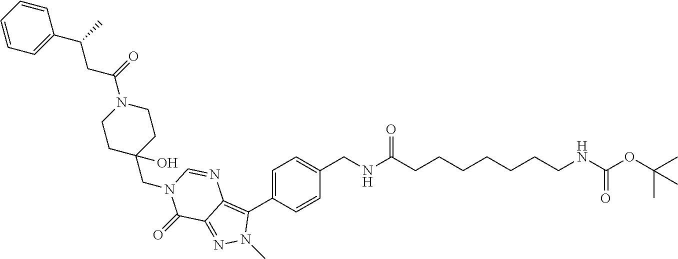 [SMILES: C[C@H](CC(=O)N1CCC(O)(Cn2cnc3c(-c4ccc(CNC(=O)CCCCCCCNC(=O)OC(C)(C)C)cc4)n(C)nc3c2=O)CC1)c1ccccc1] | U133 | *** | | *** |
| 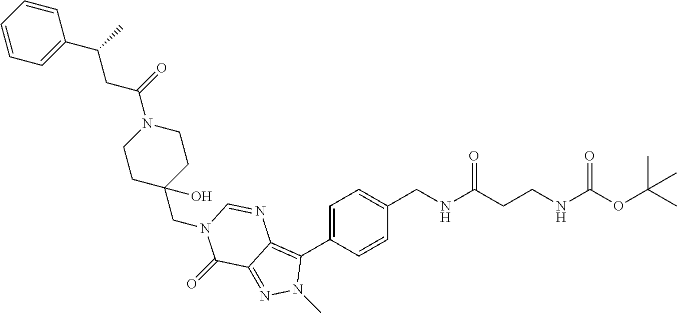 [SMILES: C[C@H](CC(=O)N1CCC(O)(Cn2cnc3c(-c4ccc(CNC(=O)CCNC(=O)OC(C)(C)C)cc4)n(C)nc3c2=O)CC1)c1ccccc1] | U134 | *** | | *** |
| 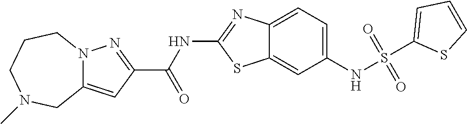 [SMILES: CN1CCCn2nc(C(=O)Nc3nc4ccc(NS(=O)(=O)c5cccs5)cc4s3)cc2C1] | U77 | ** | ** | * |
| 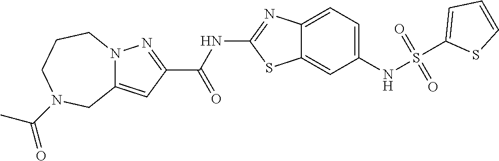 [SMILES: CC(=O)N1CCCn2nc(C(=O)Nc3nc4ccc(NS(=O)(=O)c5cccs5)cc4s3)cc2C1] | U96 | | ** | * |
| 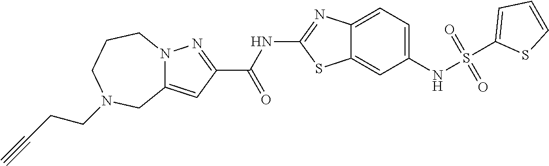 [SMILES: C#CCCN1CCCn2nc(C(=O)Nc3nc4ccc(NS(=O)(=O)c5cccs5)cc4s3)cc2C1] | U119 | | ** | * |

* is > 100 μM; ** is <100 μM and > than 1 μM; *** is < 1 μM

### Processes of Manufacture:

The protein stabilizing compound of the present invention can be manufactured according to routes described in the Working Examples below or as otherwise known in the patent or scientific literature and if appropriate supported by the knowledge of the ordinary worker or common general knowledge.

Some of the carbons in the compounds described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers—often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

TABLE 1
Abbreviations table

| Abbrev. | Name |
|---|---|
| 4-DMAP | 4-Dimethylaminopyridine |
| AcCN | acetyl cyanide |
| ACN | acetonitrile |
| AcOH, HOAc | acetic acid |
| AcONa, NaOAc | sodium acetate, |
| B₂Pin₂ | Bis(pinacolato)diboron |
| Boc₂O | Di-tert-butyl dicarbonate |
| BOP-Cl | Bis(2-oxo-3-oxazolidinyl)phosphinic chloride |
| Br₂ | bromine gas |
| BuOH | butanol |
| CbzCl | Benzyl chloroformate, |
| ClCH₂COCl | chloroacetyl chloride |
| Cs₂CO₃ | cesium carbonate |
| CuI | copper (I) iodide |
| CuSO₄ | copper (II) sulfate |
| DCE | 1,2-dichloroethane |
| DCM | dichloromethane |
| DIAD | Diisopropyl azodicarboxylate |
| DIEA, DIPEA | N,N-Diisopropylethylamine |
| DMF | Dimethylformamide |
| DMSO | dimethylsulfoxide |
| DPPA | Diphenylphosphoryl azide |
| DTT | Dithiothreitol |
| EA | ethyl acetate |
| EDCI | 1-Ethyl-3-(3-dimethylaminopropyl)carbodiimide |
| Et₃N, TEA | triethylamine |
| Et₃SiH | Triethylsilane |
| EtOH | ethanol |
| FA, HCO₂H | formic acid |
| Fe | iron |
| H₂ | hydrogen gas |
| H₂O | water |
| H₂SO₄ | sulfuric acid |
| HATU | 1-[Bis(dimethylamino)methylene]-1H- |
| | 1,2,3-triazolo[4,5-b]pyridinium 3- |
| | oxide hexafluorophosphate, |
| HCl | hydrochloric acid |
| HOAt | 1-Hydroxy-7-azabenzotriazole |
| HOBt | Hydroxybenzotriazole |
| HPLC | high performance liquid chromatography |
| K₂CO₃ | potassium carbonate |
| K₃PO₄ | potassium phosphate |
| KI | potassium iodide |
| KOAc | potassium acetate |
| KSCOCH₃ | Potassium thioacetate |
| LCMS | liquid chromatography mass spectrometry |
| m-CPBA | meta-Chloroperoxybenzoic acid |
| MeMgBr | Methylmagnesium bromide |
| MeOH | methanol |
| MsCl | Methanesulfonyl chloride |
| MTBE | methyl tert-butyl ether |
| N₂ | nitrogen gas |
| Na₂CO₃ | sodium carbonate |
| Na₂SO₃ | sodium sulfite |
| Na₂SO₄ | sodium sulfate |
| NaBH₃CN | Sodium cyanoborohydride |
| NaBH₄ | sodium borohydride |
| NaCl | sodium chloride |
| NaClO | sodium chlorite |
| NaH | sodium hydride |
| NaHCO₃ | sodium hydrogen carbonate |
| NaOH | sodium hydroxide |
| NBS | N-Bromosuccinimide |
| n-BuLi | n-Butyllithium |
| n-BuOH | butyl alcohol (1-butanol) |
| NH₂Boc, BocNH₂ | tert-Butyl carbamate; Boc-amide |
| NH₃ | ammonia |
| NH₃H₂O | ammonia solution |
| NH₄Cl | ammonium chloride |
| NHMeOMe | N,O-Dimethylhydroxylamine hydrochloride |
| NIS | N-Iodosuccinimide |
| NMM | N-Methylmorpholine |
| Pd(dppf)Cl₂ | 1,1′-Bis(diphenylphosphino) |
| | ferrocenedichloropalladium(II) |
| Pd(dtbpf)Cl₂ | [1,1′-Bis(di-tert-butylphosphino)ferrocene] |
| | dichloropalladium(II) |
| Pd(OAc)₂ | Palladium(II) acetate |
| Pd(OH)₂ | Palladium hydroxide |
| Pd(PPh₃)₂Cl₂, | Bis(triphenylphosphine)palladium chloride |
| Pd(Ph₃P)₂Cl₂ | |
| Pd(PPh₃)₄ | Tetrakis(triphenylphosphine)palladium(0) |
| Pd/C | palladium carbon |
| Pd-PEPPSI(HeptCl) | Dichloro[1,3-bis(2,6-di-4-heptylphenyl)imidazol- |
| | 2-ylidene](3-chloropyridyl)palladium(II) |
| Pd-PEPPSI-IPent | 1,3-bis[2,6-bis(1-ethylpropyl)phenyl]-2H- |
| | imidazole; 3-chloropyridine; dichloropalladium |
| PE | petroleum ether |
| PMA | para-Methoxyamphetamine |
| PPh₃, Ph₃P | Triphenylphosphine |
| PyBOP | (benzotriazol-1-yloxytripyrrolidinophosphonium |
| | hexafluorophosphate) |
| Sat. | saturated |
| SFC | Supercritical fluid chromatography |
| SiO₂ | silicon dioxide |
| SOCl₂ | thionyl chloride |
| SPDP | succinimidyl 3-(2-pyridyldithio)propionate |
| TBAF | Tetra-n-butylammonium fluoride |
| TBD | Triazabicyclodecene |
| t-BiXphos-Pd-G3 | [(2-Di-tert-butylphosphino-2′,4′,6′-triisopropyl-1, |
| | 1′-biphenyl)-2-(2′-amino-1,1′-biphenyl)] |
| | palladium(II) methanesulfonate |
| t-BuNH₂ | tert-Butylamine |
| TEA | triethylamine |
| TFA | trifluoroacetic acid |
| THF | tetrahydrofuran |
| THPTA | tris-hydroxypropyltriazolylmethylamine |
| TLC | thin layer chromatography |
| TMPMgCl | 2,2,6,6 Tetramethylpiperidinylmagnesium |
| | chloride |
| tol., PhCH₃ | toluene |
| TSO₂ | thiophene sulfone |

### Example 1. General Schemes

The compounds of the present invention can by synthesized in a modular manner using techniques known to the skilled artisan. Provided in this example are general strategies for linking a USP7 Targeting Ligand described herein to a Ubiquitinated Protein Targeting Ligand described herein. These strategies can be used to install multiple linking moieties together (for example Linker-A and Linker-B) in a stepwise fashion. The reagents listed in this example are non-limiting reagents to perform routine chemical reactions and can be readily substituted for other reagents known in the art as desired.

### Example 1A. Attachment of Triazole-Containing Alkyl or Polyethylene Glycol Chains as Linker

### For Linear Alkyl:

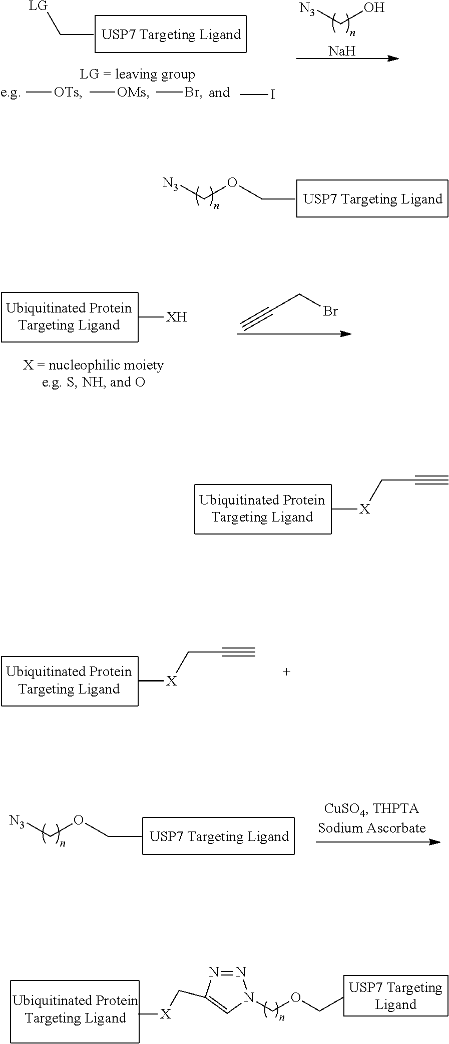

### For Polyethylene Glycol:

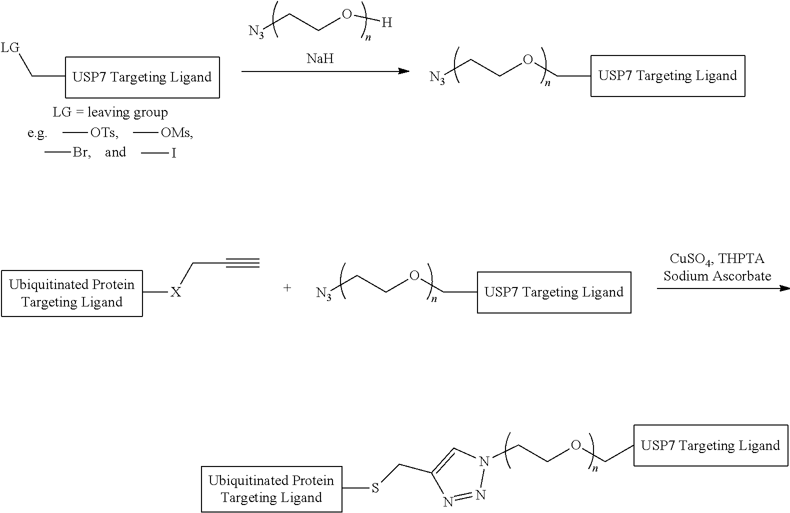

In certain embodiments, the reactive groups on the ligands shown herein are switched. For example, the nucleophilic moiety is bonded to the USP7 Targeting Ligand and the leaving group is on the Ubiquitinated Protein Targeting Ligand.

### Example 1B. Attachment of Succinimide-Containing Groups as Linker

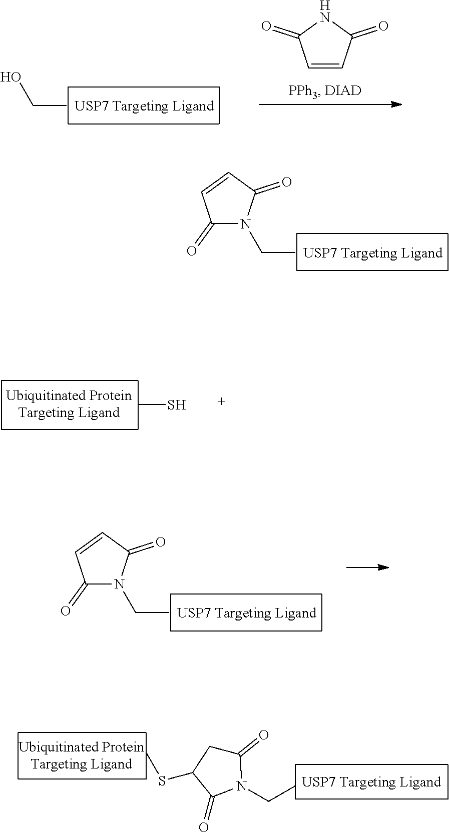

In certain embodiments, the reactive groups on the ligands shown herein are switched. For example, the electrophilic maleimide moiety is bonded to the Ubiquitinated Protein Targeting Ligand and the nucleophilic moiety is on the USP7 Targeting Ligand.

### Example 1C. Attachment of Amide-Containing Alkyl or Polyethylene Glycol Chains as Linker

### For Linear Alkyl:

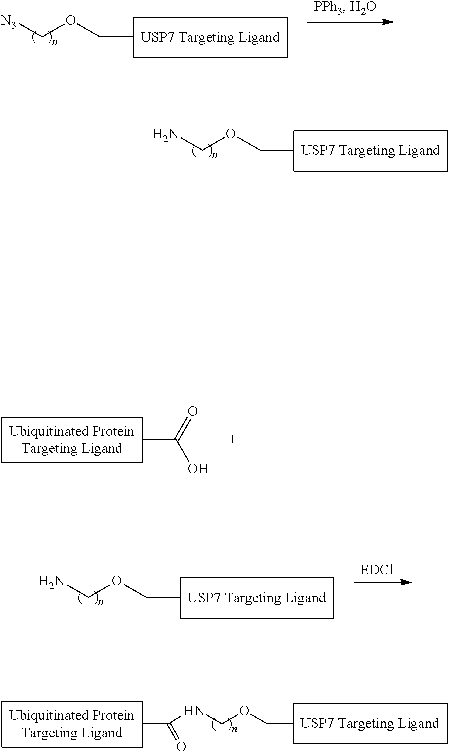

### For Polyethylene Glycol:

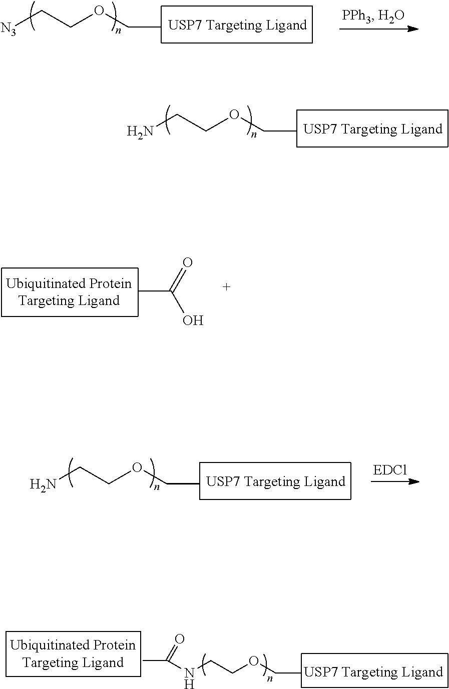

In certain embodiments, the reactive groups on the ligands shown herein are switched. For example, the amine moiety is bonded to the Ubiquitinated Protein Targeting Ligand and the carboxylic acid moiety is on the USP7 Targeting Ligand.

### Example 1D Attachment of Triazole-Containing Alkyl or Polyethylene Glycol Chains as Linker-A or Linker-B

### Linear Alkyl as Linker-A:

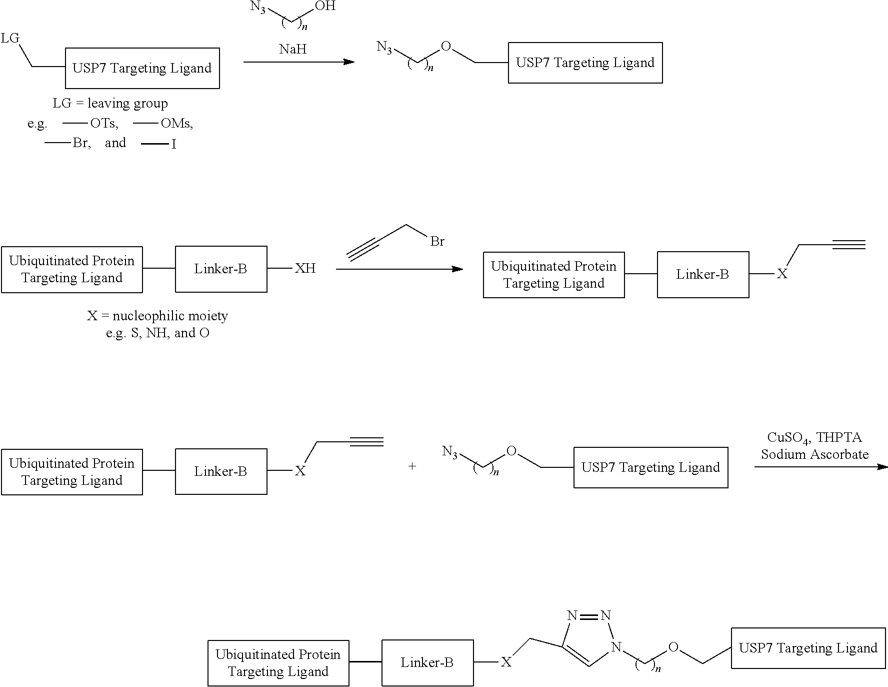

In certain embodiments, the reactive groups on the ligands shown herein are switched. For example, the leaving group moiety is bonded to the Ubiquitinated Protein Targeting Ligand and the nucleophilic moiety is on the Linker-B.

### Alternatively for Linear Alkyl as Linker-B:

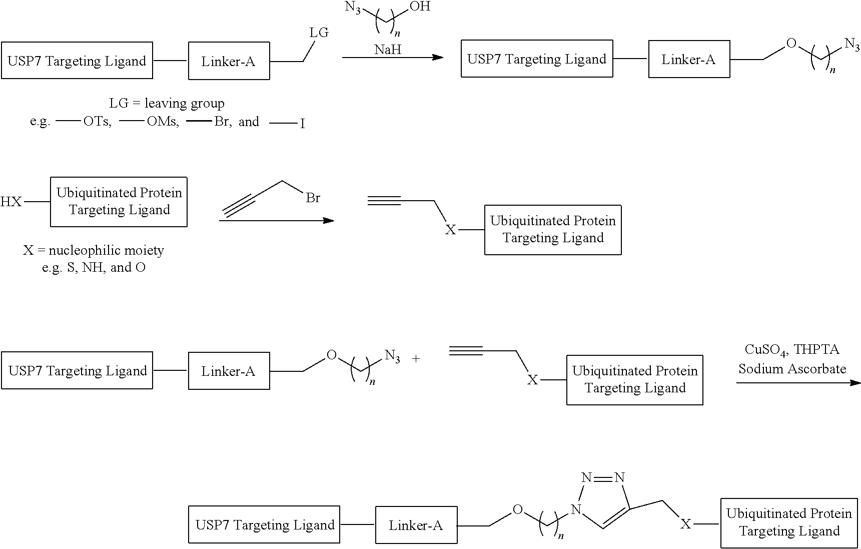

In certain embodiments, the reactive groups on the ligands shown herein are switched. For example, the leaving group moiety is bonded to the Ubiquitinated Protein Targeting Ligand and the nucleophilic moiety is on the Linker-A.

### For Polyethylene Glycol as Linker-A:

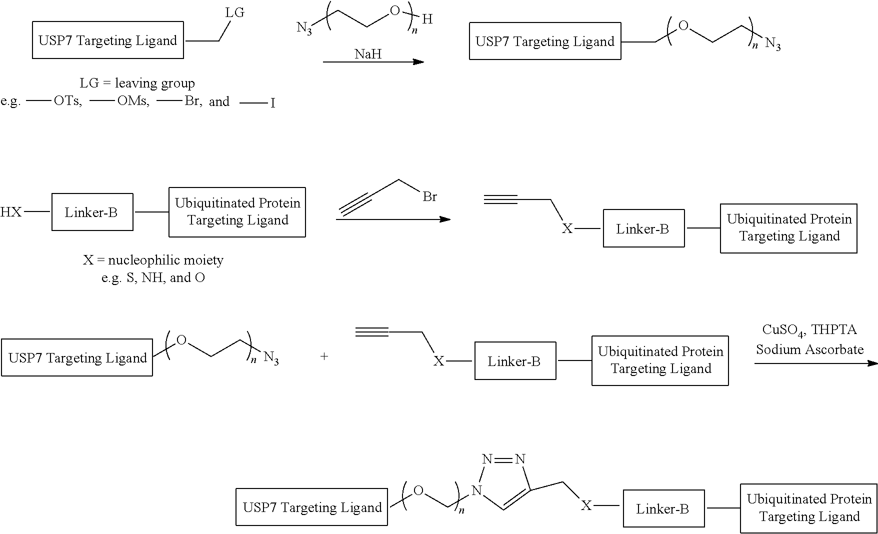

In certain embodiments, the reactive groups on the ligands shown herein are switched. For example, the leaving group moiety is bonded to the Linker-B and the nucleophilic moiety is on the USP7 Targeting Ligand.

### Alternatively, for Polyethylene Glycol as Linker-B:

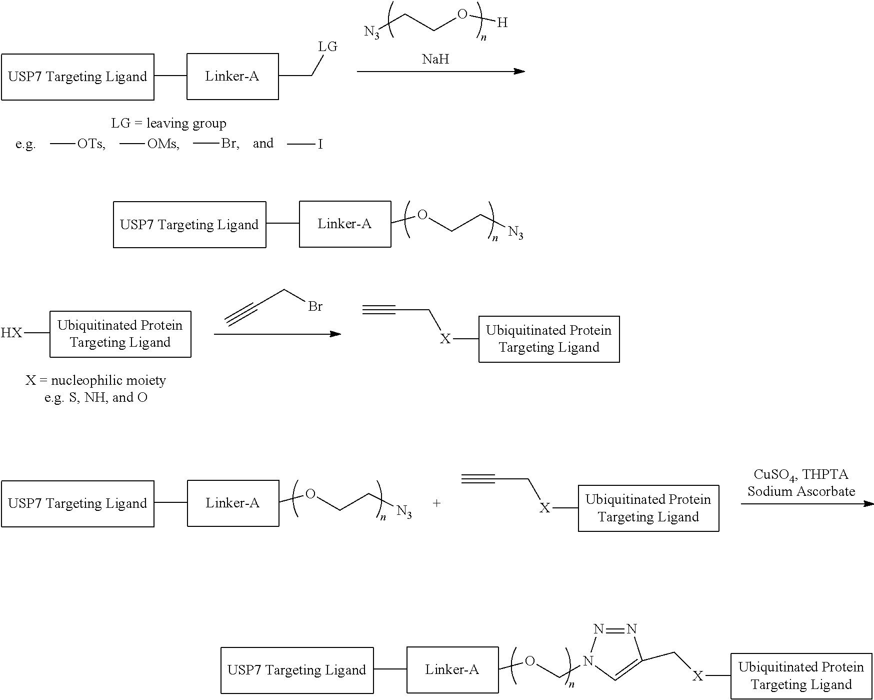

In certain embodiments, the reactive groups on the ligands shown herein are switched. For example, the leaving group moiety is bonded to the Ubiquitinated Protein Targeting Ligand and the nucleophilic moiety is on the Linker-A.

### Example 1E. Attachment of Succinimide-Containing Groups as Linker-A or Linker-B

### Succinimide-Containing Group as Linker-A:

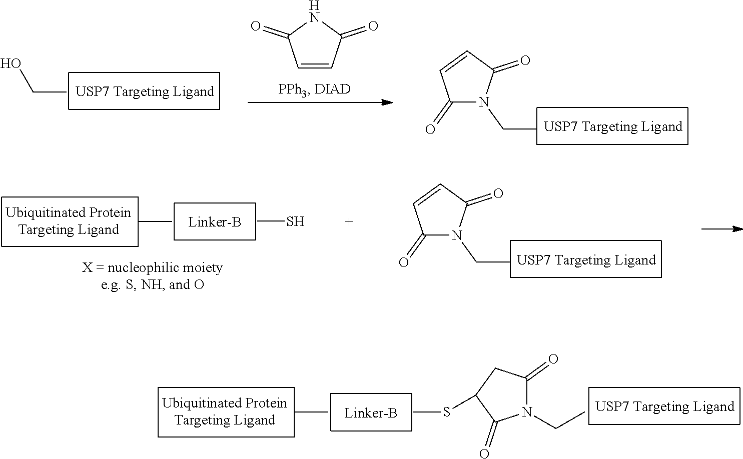

In certain embodiments, the reactive groups on the ligands shown herein are switched. For example, the electrophilic maleimide moiety is bonded to the Linker-B and the nucleophilic moiety is on the USP7 Targeting Ligand.

### Succinimide-Containing Group as Linker-B:

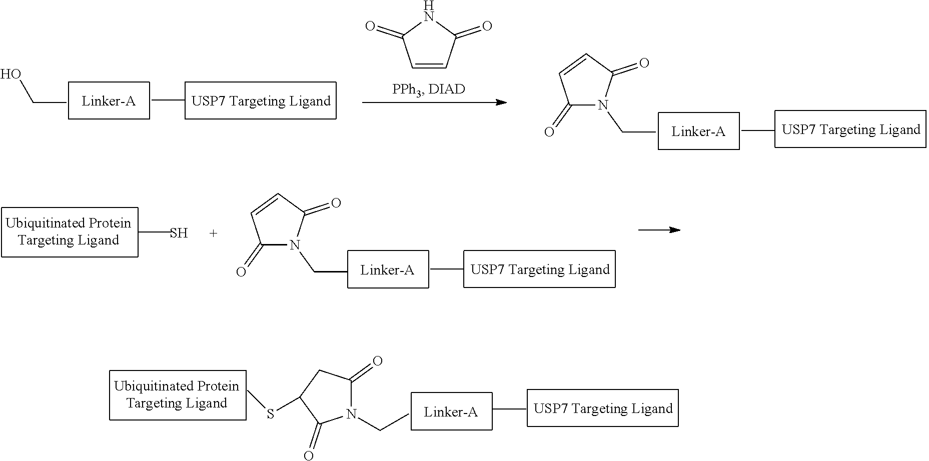

In certain embodiments, the reactive groups on the ligands shown herein are switched. For example, the electrophilic maleimide moiety is bonded to the Ubiquitinated Protein Targeting Ligand and the nucleophilic moiety is on the Linker-A.

### Example 1F. Attachment of Amide-Containing Alkyl or Polyethylene Glycol Chains as Linker

### For Linear Alkyl as Linker-A:

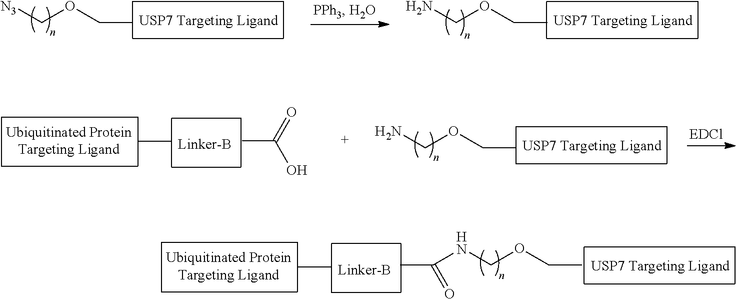

### For Polyethylene Glycol as Linker-A:

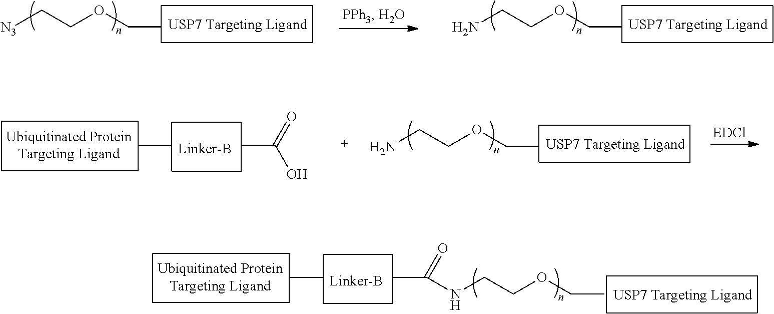

### For Linear Alkyl as Linker-B:

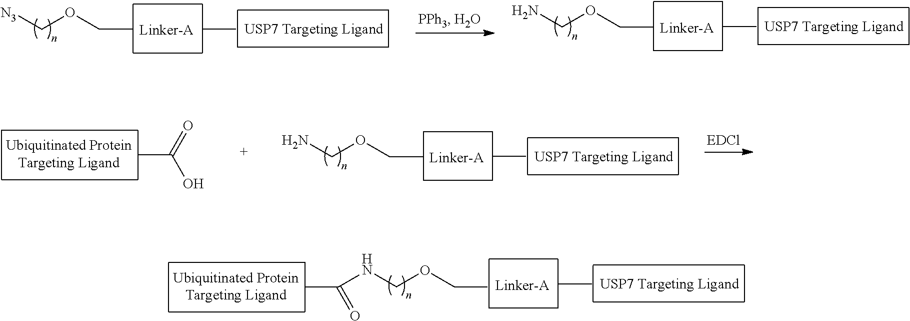

### For Polyethylene Glycol as Linker-B:

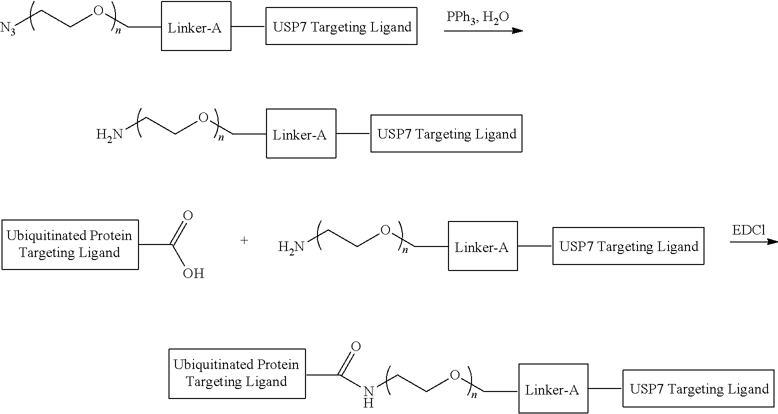

### Example 1G. Attachment Point of Linker

The compounds of the present invention can be prepared using a desired attachment point linking the Ubiquitinated Protein Targeting Ligand by preparing or procuring appropriate starting materials with corresponding functionality. For example,

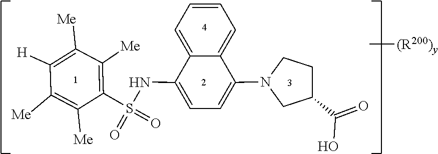

when attached to the Linker in the cycle marked with a 1 includes the following non-limiting exemplary structure:

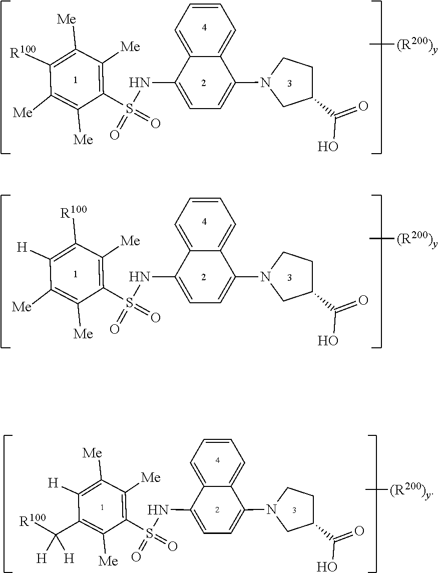

The synthesis of this KEAP1 Targeting Ligand has been reported in the literature. For example in Journal of Medicinal Chemistry (2019), 62(17), 8028-8052:

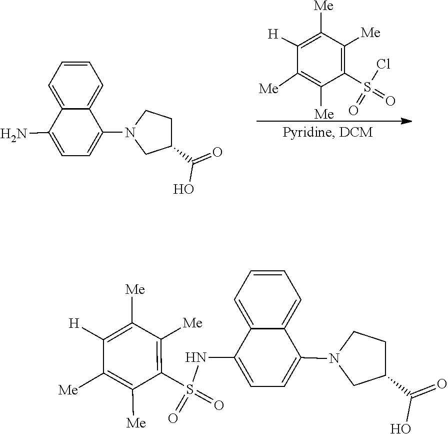 [SMILES: Nc1ccc(N2CC[C@H](C(=O)O)C2)c2ccccc12 ; Cc1cc(C)c(C)c(S(=O)(=O)Cl)c1C ; Cc1cc(C)c(C)c(S(=O)(=O)Nc2ccc(N3CC[C@H](C(=O)O)C3)c3ccccc23)c1C]

The starting materials in this synthesis can be replaced as necessary to provide functional groups that can be linked at the cycle 1 position. For example:

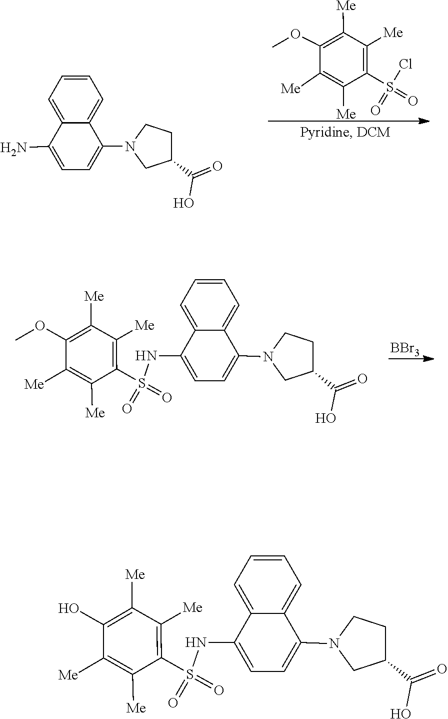 [SMILES: Nc1ccc(N2CC[C@H](C(=O)O)C2)c2ccccc12 ; COc1c(C)c(C)c(S(=O)(=O)Cl)c(C)c1C ; COc1c(C)c(C)c(S(=O)(=O)Nc2ccc(N3CC[C@H](C(=O)O)C3)c3ccccc23)c(C)c1C ; "BBr3" ; Cc1c(C)c(S(=O)(=O)Nc2ccc(N3CC[C@H](C(=O)O)C3)c3ccccc23)c(C)c(C)c1O]

Additional transformations can be employed as needed to use other linking locations. For example:

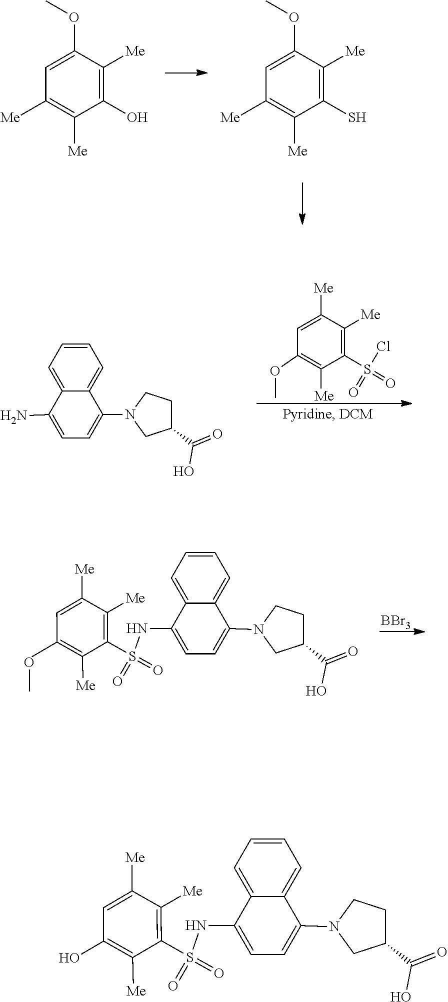

These techniques as well as other well-known reactions such as nucleophilic substitutions and coupling reactions can be used to prepare compounds that are linked differently to cycle 1 than those described above. Additional non-limiting examples of starting materials that can be employed to attach a linker to cycle 1 include:

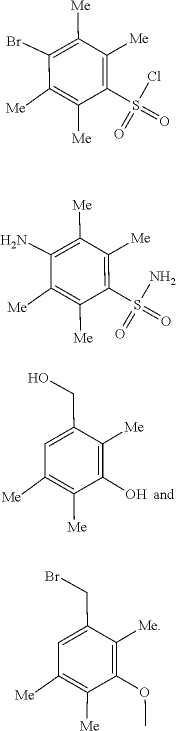

### Example 1H: General Methods

### LCMS Analysis Condition:

Instrument name: Agilent Technologies 1290 infinity 11.

Method A: Method: A—0.1% Formic Acid in H₂O, B—0.1% FA in ACN; flow rate: 2.0 mL/min; column: XBridge C8 (50×4.6 mm, 3.5 μm), +ve and −ve mode

Method B: Method: A—0.1% TFA in H₂O, B—0.1% TFA in ACN; flow rate: 2.0 mL/min; column: XBridge C8 (50×4.6 mm, 3.5 μm), +ve mode

Method C: Method: A—10 mM NH₄HCO₃ in H₂O, B—ACN; flow rate: 1.0 mL/min; column: XBridge C8 (50×4.6 mm, 3.5 μm), +ve and −ve mode

### HPLC Analysis Condition:

Instrument name: Agilent 1200 Series instruments as followed using % with UV detection (maxplot).

Method A: Method: A—0.1% TFA in H₂O, B—0.1% TFA in ACN; flow rate: 2.0 mL/min; column: XBridge C8 (50×4.6 mm, 3.5 μm).

Method B: Method: A—0.1% Formic acid in H₂O, B-ACN; flow rate: 2.0 mL/min; column: XBridge C8 (50×4.6 mm, 3.5 μm).

Method C: Method: A—10 mM ammonium bicarbonate in H₂O, B-ACN; flow rate: 1.0 mL/min; column: XBridge C8 (50×4.6 mm, 3.5 μm).

### Example 2. Synthesis of tert-butyl (2-(3-(1,3-dioxo-3,4-dihydroisoquinolin-2(1H)-yl)propanamido)ethyl)carbamate (Intermediate 2-3)

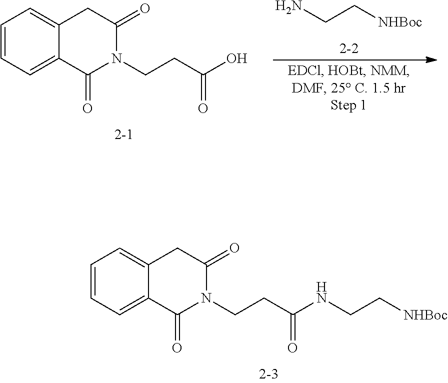 [SMILES: O=C(O)CCN1C(=O)Cc2ccccc2C1=O ; CC(C)(C)OC(=O)NCCN ; CC(C)(C)OC(=O)NCCNC(=O)CCN1C(=O)Cc2ccccc2C1=O]

Step 1: To a solution of Intermediate 2-1 (0.5 g, 2.14 mmol, 1 eq) in DMF (5 mL) was added EDCI (821.98 mg, 4.29 mmol, 2 eq) and HOBt (144.85 mg, 1.07 mmol, 0.5 eq) and NMM (1.08 g, 10.72 mmol, 1.18 mL, 5 eq) stirred 0.5 hr at 25° C., then added Intermediate 2-2 (412.18 mg, 2.57 mmol, 404.10 μL, 1.2 eq) in the mixture was stirred at 25° C. for 1 hr. LCMS showed desired molecular weight was detected. The mixture was washed with water (5 mL) and extracted with EA (10 mL*3). The combined organic layers were dried over anhydrous sodium sulfate and concentrated under reduced pressure to give crude product. The residue was purified by prep-HPLC (FA condition) and lyophilized to afford Intermediate 2-3 (0.35 g, 932.29 μmol, 43.49% yield, 100% purity) as a pink solid and confirmed by LCMS and HNMR. Mass Found LCMS: Retention time: 0.784 min, (M+H−100)=276.1; LCMS: Retention time: 0.824 min, (M+H)=376.0; ¹H NMR (400 MHz, DMSO-d₆) δ=8.05 (d, J=7.6 Hz, 1H), 7.97-7.89 (m, 1H), 7.71-7.63 (m, 1H), 7.52-7.44 (m, 1H), 7.40 (d, J=7.6 Hz, 1H), 6.78-6.74 (m, 1H), 4.14 (s, 2H), 4.10-4.00 (m, 2H), 3.07-3.01 (m, 2H), 2.98-2.93 (m, 2H), 2.36-2.31 (m, 2H), 1.38 (s, 9H).

### Example 3. Synthesis of tert-butyl (2-(3-(1,3-dioxo-3,4-dihydroisoquinolin-2(1H)-yl)-N-methoxypropanamido)ethyl)carbamate (Intermediate 3-5)

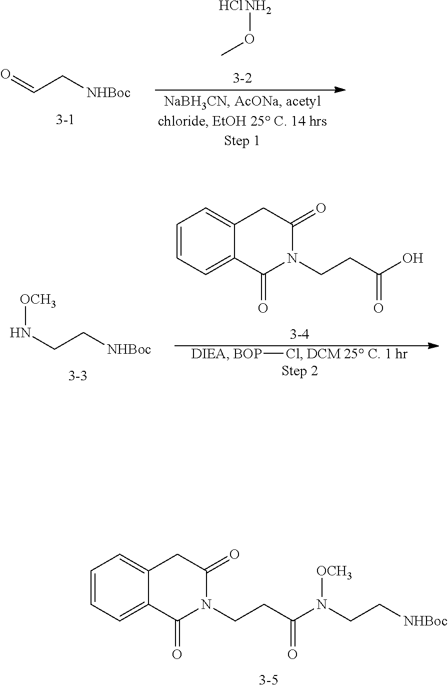

Step 1: To a solution of Intermediate 3-1 (2 g, 12.56 mmol, 1 eq) in EtOH (20 mL) was added Intermediate 3-2 (1.26 g, 15.08 mmol, 1.2 eq, HCl) and AcONa (2.06 g, 25.13 mmol, 2 eq), the mixture was stirred at 25° C. for 12 hrs. A solution of acetyl chloride (3.15 g, 40.08 mmol, 2.86 mL, 3.19 eq) in EtOH (40 mL) was added into the reaction and added NaBH₃CN (789.56 mg, 12.56 mmol, 1 eq), then the mixture was stirred at 25° C. for 2 hrs. TLC indicated one major new spot was detected. The mixture was concentrated to give Intermediate 3-3 (2.5 g, crude) as colorless oil and confirmed by HNMR (400 MHz, DMSO-d₆) δ=3.37 (s, 3H), 2.81-2.77 (m, 2H), 2.73-2.66 (m, 2H), 0.91 (s, 9H).

Step 2: To a solution of Intermediate 3-4 (0.05 g, 214.39 μmol, 1 eq) in DCM (0.5 mL) was added DIEA (83.13 mg, 643.17 μmol, 112.03 μL, 3 eq) and BOP—Cl (65.49 mg, 257.27 μmol, 1.2 eq) and the mixture was stirred 0.5 hr at 25° C., then added Intermediate 3-3 (48.94 mg, 257.27 μmol, 404.10 μL, 1.2 eq) in the mixture and stirred at 25° C. for 0.5 hr. LCMS showed desired mass was detected. The mixture was washed with water (0.5 mL) and extracted with DCM (1 ml*3), the organic layers were dried over anhydrous Na₂SO₄, filtered and concentrated to give a crude product. The residue was purified by prep-HPLC (column: Phenomenex Synergi C18 150*25 mm*10 μm; mobile phase: [water (0.225% FA)-ACN]; B %: 30%-60%, 10 min) and lyophilized to afford Intermediate 3-5 (0.01 g, 24.54 μmol, 11.45% yield, 99.497% purity) as yellow oil and confirmed by LCMS and HNMR. Mass Found, LCMS: Retention time: 0.811 min, (M+H−100)=306.1; LCMS: Retention time: 0.876 min, (M+H−100)=306.2; 1H NMR (400 MHz, Chloroform-d) δ=8.14 (d, J=8.0 Hz, 1H), 7.54-7.49 (m, 1H), 7.40-7.33 (m, 1H), 7.21 (s, 1H), 5.14 (s, 1H), 4.27 (t, J=7.2 Hz, 2H), 3.99 (s, 2H), 3.64 (t, J=5.6 Hz, 2H), 3.60 (s, 3H), 3.30-3.25 (m, 2H), 2.70 (t, J=7.2 Hz, 2H), 1.38 (s, 9H).

### Example 4. Synthesis of tert-butyl (2-((3-(3-(1,3-dioxo-3,4-dihydroisoquinolin-2(1H)-yl)propanamido)phenyl)amino)ethyl)carbamate (Intermediate 4-5)

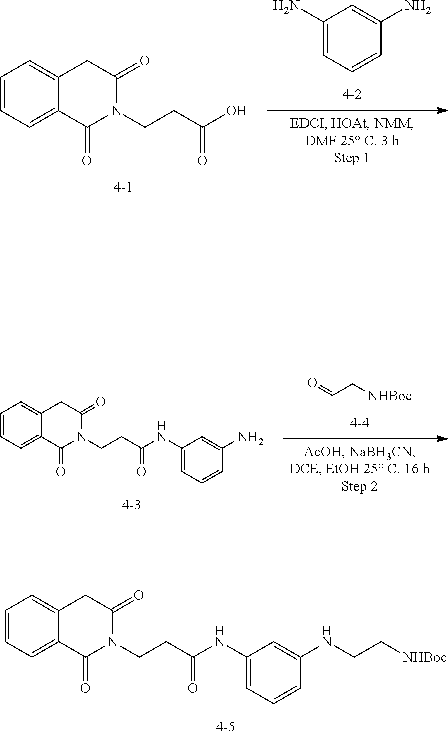

Step 1: To a solution of Intermediate 4-1 (500 mg, 2.14 mmol, 1 eq) and Intermediate 4-2 (1.16 g, 10.72 mmol, 5 eq) in DMF (5 mL) was added EDCI (821.98 mg, 4.29 mmol, 2 eq), NMM (1.08 g, 10.72 mmol, 1.18 mL, 5 eq) and HOAt (145.90 mg, 1.07 mmol, 149.95 μL, 0.5 eq). The mixture was stirred at 25° C. for 16 hr. LCMS showed Intermediate 4-1 was consumed completely and one major peak with desired mass was detected. The mixture was diluted with H₂O 10 mL and extracted with EA 60 mL (20 mL*3). The combined organic layers were washed with Sat. NaCl 10 mL, dried over Na₂SO₄, filtered and concentrated under reduced pressure to give a residue. The crude product was purified by reverse-phase HPLC (0.1% FA condition). The eluent was lyophilized to afford product. Intermediate 4-3 (500 mg, 1.55 mmol, 72.13% yield) was obtained as a black solid, which was confirmed by HNMR. Mass Found, LCMS: Retention time: 0.593 min, (M+H)=324.1; 1H NMR (400 MHz, DMSO-d₆) δ=9.68 (s, 1H), 8.19 (d, J=0.6 Hz, 1H), 8.05 (d, J=7.8 Hz, 1H), 7.68-7.64 (m, 1H), 7.53-7.43 (m, 1H), 7.39 (d, J=7.6 Hz, 1H), 6.97-6.84 (m, 2H), 6.64 (s, 1H), 6.30-6.16 (m, 1H), 4.24-4.03 (m, 4H), 3.60-3.49 (m, 2H).

Step 2: To a mixture of Intermediate 4-3 (500 mg, 1.55 mmol, 1 eq), Intermediate 4-4 (246.15 mg, 1.55 mmol, 1 eq) and AcOH (92.86 mg, 1.55 mmol, 88.44 μL, 1 eq) in DCE (5 mL) and EtOH (2.5 mL) was stirred at 25° C. for 0.5 hr. Then NaBH₃CN (388.70 mg, 6.19 mmol, 4 eq) was added to the mixture and stirred at 25° C. for 15.5 hrs. LCMS showed 14% of desired mass was detected and 12% of reactant 1 remained. The mixture was diluted with H₂O 20 mL and extracted with EA 60 mL (20 mL*3). The combined organic layers were washed with Sat. NaCl 30 mL, dried over Na₂SO₄, filtered and concentrated under reduced pressure to give a residue. The residue was purified by prep-HPLC (column: Phenomenex Synergi C18 150*25 mm*10 μm; mobile phase: [water (0.225% FA)-ACN]; B %: 40%-60%, 10 min). The eluent was lyophilized to afford Intermediate 4-5 (40 mg, 81.45 μmol, 5.27% yield, 95% purity) was obtained as an off-white solid, which was confirmed by LCMS and HNMR. Mass Found, LCMS: Retention time: 0.790 min, (M+H)=467.1; LCMS: Retention time: 0.790 min, (M+H)=467.2; 1H NMR (400 MHz, DMSO-d₆) δ=9.70 (s, 1H), 8.09-8.02 (m, 1H), 7.68-7.64 (m, 1H), 7.52-7.45 (m, 1H), 7.40 (d, J=7.6 Hz, 1H), 6.97-6.93 (m, 1H), 6.91-6.82 (m, 2H), 6.69 (br d, J=8.4 Hz, 1H), 6.26-6.24 (m, 1H), 5.56 (br d, J=5.2 Hz, 1H), 4.18-4.07 (m, 4H), 3.11-3.06 (m, 2H), 3.04-2.95 (m, 2H), 2.58-2.53 (m, 2H), 1.38 (s, 9H).

### Example 5. Synthesis of 4-(6-amino-5-(4-(2-azidoethoxy) phenyl)-4-ethylpyridin-3-yl) phenol (Intermediate 5-3)

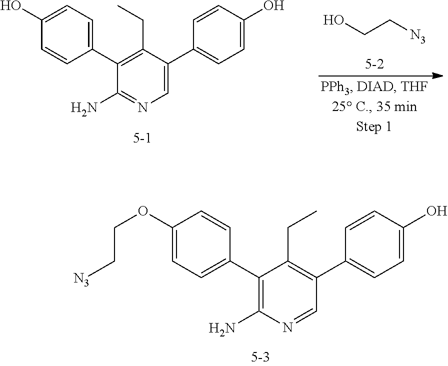 [SMILES: CCc1c(-c2ccc(O)cc2)cnc(N)c1-c1ccc(O)cc1 ; [N-]=[N+]=NCCO ; CCc1c(-c2ccc(O)cc2)cnc(N)c1-c1ccc(OCCN=[N+]=[N-])cc1]

Step 1: To a solution of PPh₃ (47.09 mg, 179.53 μmol, 1.1 eq) in THE (0.05 mL) was added DIAD (36.30 mg, 179.53 μmol, 34.91 μL, 1.1 eq) and stirred for 5 min at 20° C. until yellow precipitate formed. To a solution of Intermediate 5-1 (0.05 g, 163.21 μmol, 1 eq) and Intermediate 5-2 (15.63 mg, 179.53 μmol, 1.1 eq) in THE (0.2 mL) was added to the mixture and the resulting mixture was sonicated at 25° C. for 30 min. LCMS showed desired molecular weight was detected. The reaction mixture was added H₂O (5 mL) and then extracted with EA (10 mL*3), the combined organic phase was washed with brine (10 mL), dried by Na₂SO₄, filtered and concentrated to give residue was purified by prep-HPLC (column: Phenomenex Synergi C18 150*25 mm*10 um; mobile phase: [water (0.225% FA)-ACN];B %: 11%-41%, 10 min) the eluent was concentrated to remove ACN and lyophilized to afford Intermediate 5-3 (4.54 mg, 12.09 μmol, 7.41% yield, 100% purity) as a white solid and confirmed by HNMR, 2D NMR and LCMS. Mass Found, LCMS: Retention time: 0.756 min, (M+H)=376.1, and LCMS: Retention time: 0.798 min, (M+H)=376.0; NMR Data, 1H NMR (400 MHz, DMSO-d₆) δ=7.67 (s, 1H), 7.22-7.17 (m, 2H), 7.12-7.05 (m, 4H), 6.82-6.76 (m, 2H), 4.93 (s, 2H), 4.22 (t, J=4.8 Hz, 2H), 3.71-3.66 (m, 2H), 2.23-2.20 (m, 2H), 0.62-0.59 (m, 3H).

### Example 6. Synthesis of tert-butyl (2-(4-(6-amino-4-ethyl-5-(4-hydroxyphenyl) pyridin-3-yl)phenoxy)ethyl) carbamate (Intermediate 6-2) and tert-butyl (2-(4-(2-amino-4-ethyl-5-(4-hydroxyphenyl)pyridin-3-yl)phenoxy)ethyl)carbamate (Intermediate 6-3) and di-tert-butyl ((((2-amino-4-ethylpyridine-3,5-diyl)bis(4,1-phenylene))bis(oxy))bis(ethane-2,1-diyl))dicarbamate (Intermediate 6-4)

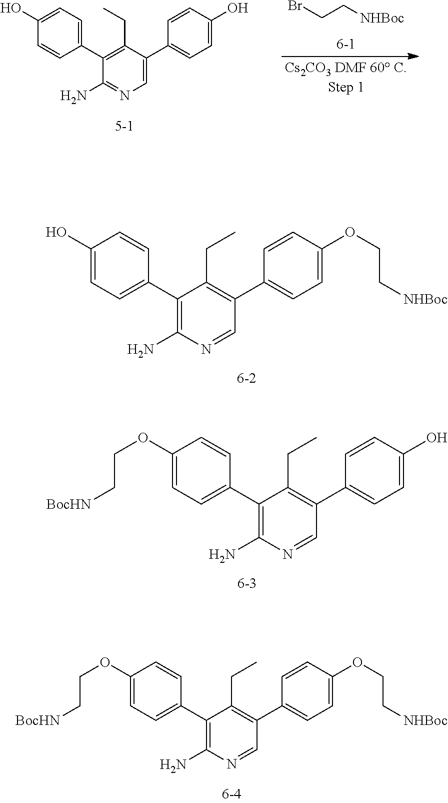 [SMILES: CCc1c(-c2ccc(O)cc2)cnc(N)c1-c1ccc(O)cc1 ; CC(C)(C)OC(=O)NCCBr ; CCc1c(-c2ccc(OCCNC(=O)OC(C)(C)C)cc2)cnc(N)c1-c1ccc(O)cc1 ; CCc1c(-c2ccc(O)cc2)cnc(N)c1-c1ccc(OCCNC(=O)OC(C)(C)C)cc1 ; CCc1c(-c2ccc(OCCNC(=O)OC(C)(C)C)cc2)cnc(N)c1-c1ccc(OCCNC(=O)OC(C)(C)C)cc1]

Step 1: To a solution of Intermediate 5-1 (0.05 g, 163.21 μmol, 1 eq) and Intermediate 6-1 (36.57 mg, 163.21 μmol, 1 eq) in DMF (0.5 mL) was added Cs₂CO₃ (106.35 mg, 326.42 μmol, 2 eq). The mixture was stirred at 60° C. for 3 hrs. LCMS showed Intermediate 5-1 was consumed completely and one mainly peak with desired mass was detected. The reaction mixture was added H₂O (5 mL) and then extracted with EA (10 mL*3), the combined organic phase was washed with brine (10 mL), dried by Na₂SO₄, filtered and concentrated. The residue was purified by prep-HPLC (column: Phenomenex Synergi C18 150*25 mm*10 μm; mobile phase: [water (0.225% FA)-ACN]; B %: 13%-46%, 11 min). Intermediate 6-4 (0.01 g, 16.87 μmol, 10.34% yield) was obtained as a white solid and by prep-HPLC (column: Phenomenex Synergi C18 150*25 mm*10 um; mobile phase: [water (0.225% FA)-ACN]; B %: 23%-53%, 10 min), the eluent was concentrated to remove ACN and lyophilized to afford Intermediate 6-4 (3.89 mg, 6.56 μmol, 38.90% yield) as a white solid and confirmed by HNMR and LCMS. Intermediate 6-2 and Intermediate 6-3 (0.01 g, 22.24 μmol, 13.63% yield, mixture) was obtained as a white solid. The crude product was purified by SFC (column: DAICEL CHIRALPAK AD (250 mm*30 mm, 10 μm); mobile phase: [0.1% NH₃H₂O MEOH]; B %: 40%-40%, 4.6 min; 30 min) and the eluent was concentrated to afford Intermediate 6-2 (5.11 mg, 11.37 μmol, 5.11% yield) as yellow oil and confirmed by HNMR, LCMS and 2D NMR. Intermediate 6-3 (7.35 mg, 16.35 μmol, 7.35% yield) was obtained as yellow oil and confirmed by HNMR, LCMS and 2D NMR.

Mass Found, LCMS: Retention time: 0.786 min 0.872 min, (M+H)=450.2, 593.3, LCMS: Retention time: 0.902 min, (M+H)=593.4, LCMS: Retention time: 0.902 min, (M+H)=450.4, and LCMS: Retention time: 0.902 min, (M+H)=450.4; NMR Data, 1H NMR (400 MHz, DMSO-d₆) δ=9.65-9.40 (m, 1H), 7.66 (s, 1H), 7.26-7.17 (m, 2H), 7.08-7.02 (m, 2H), 6.99-6.93 (m, 2H), 6.92-6.84 (m, 2H), 4.93 (s, 2H), 4.03-3.94 (m, 2H), 3.32-3.27 (m, 2H), 2.24-2.20 (m, 2H), 1.38 (s, 9H), 0.61-0.59 (m, 3H); 1H NMR (400 MHz, DMSO-d₆) δ=9.48-9.30 (m, 1H), 7.63 (s, 1H), 7.21-7.13 (m, 2H), 7.11-7.07 (m, 2H), 7.06-7.02 (m, 2H), 6.79 (d, J=8.4 Hz, 2H), 4.92 (s, 2H), 4.00-3.98 (m, 2H), 3.33-3.29 (m, 2H), 2.23-2.20 (m, 2H), 1.39 (s, 9H), 0.61-0.59 (m, 3H); 1H NMR (400 MHz, DMSO-d₆) δ=8.41 (s, 1H), 7.68 (s, 1H), 7.19-7.15 (m, 4H), 7.07-7.03 (m, 2H), 7.03-7.00 (m, 1H), 6.99-6.94 (m, 2H), 4.96 (s, 2H), 4.05-3.94 (m, 4H), 3.32 (d, J=6.4 Hz, 4H), 2.28-2.18 (m, 2H), 1.39 (s, 18H), 0.61-0.59 (m, 3H). SFC Data, SFC: Retention time: 1.816 min, SFC: Retention time: 2.276 min.

### Example 7. Synthesis of N-(2-bromophenyl)-3-(1,3-dioxo-3,4-dihydroisoquinolin-2(1H)-yl)propanamide (Intermediate 7-6)

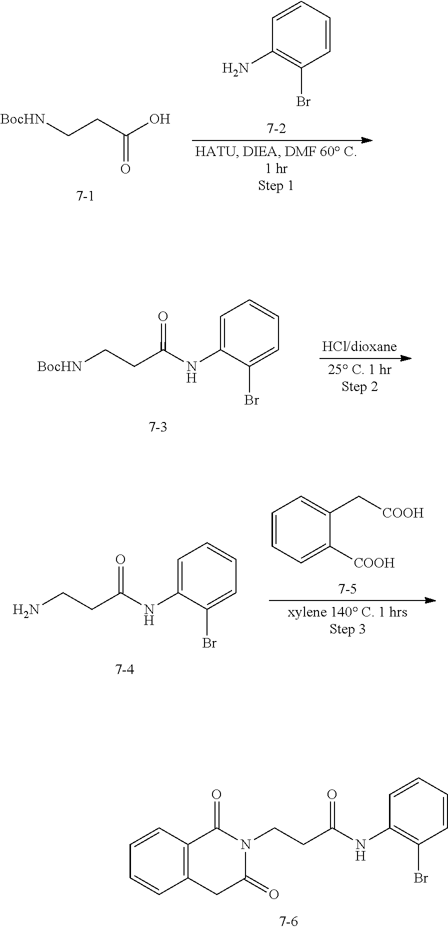

Step 1: To a solution of Intermediate 7-2 (2 g, 11.63 mmol, 404.10 μL, 1 eq) and Intermediate 7-1 (2.20 g, 11.63 mmol, 1 eq) in DMF (15 mL) was added HATU (8.84 g, 23.25 mmol, 2 eq) and DIEA (3.01 g, 23.25 mmol, 4.05 mL, 2 eq) at 25° C., then the mixture was stirred at 60° C. for 1 hr. LCMS showed desired molecular weight was detected. The mixture was washed with water (15 mL) and extracted with DCM (20 mL*3). The combined organic layers were dried over anhydrous sodium sulfate and concentrated under reduced pressure to give crude product. The residue was purified by flash silica gel chromatography (ISCO®; 40 g SepaFlash® Silica Flash Column, Eluent of 0-50% EA/PE, PE/EA=3:1, Rf=0.6) and the eluent was concentrated to give Intermediate 7-3 (3 g, 8.74 mmol, 75.18% yield, N/A purity) as white solid and confirmed by LCMS. Mass Found LCMS: Retention time: 0.831 min, (M+H−100)=243.1, and LCMS: Retention time: 0.813 min, (M+H−100)=243.0.

Step 2: To a solution of Intermediate 7-3 (600 mg, 1.75 mmol, 1 eq) in HCl/dioxane (4 M, 6 mL, 13.73 eq), then the mixture was stirred at 25° C. for 1 hr. LCMS showed desired molecular weight was detected. The mixture was concentrated to give Intermediate 7-4 (0.48 g, 1.72 mmol, 98.22% yield, HCl) as white solid without further purification. Mass Found, LCMS: Retention time: 0.345 min, (M+H)=243.0.

Step 3: The solution of Intermediate 7-5 (0.33 g, 1.70 mmol, 1 eq) and Intermediate 7-4 (475.09 mg, 1.70 mmol, 404.44 μL, 1 eq, HCl) in xylene (8 mL) was stirred at 140° C. for 1 hr. LCMS showed desired molecular weight was detected. The mixture was concentrated to give crude product. The residue was purified by prep-HPLC (column: Phenomenex Synergi C18 150*25 mm*10 um; mobile phase: [water (0.225% FA)-ACN]; B %: 38%-68%, 10 min) and lyophilized to afford Intermediate 7-6 (0.132 g, 340.89 μmol, 20.06% yield, 100% purity) was white solid and confirmed by LCMS and HNMR. Mass Found LCMS: Retention time: 0.822 min, (M+H)=387.1, and LCMS: Retention time: 0.875 min, (M+H)=388.8; ¹H NMR (400 MHz, DMSO-d₆) δ=9.57 (s, 1H), 8.07 (d, J=7.6 Hz, 1H), 7.70-7.62 (m, 2H), 7.54 (d, J=7.6 Hz, 1H), 7.52-7.46 (m, 1H), 7.43-7.34 (m, 2H), 7.17-7.10 (m, 1H), 4.21-4.13 (m, 4H), 2.64 (t, J=6.4 Hz, 2H).

### Example 8. Synthesis of (R)-7-bromo-3-((4-hydroxy-1-(3-phenylbutanoyl)piperidin-4-yl)methyl)thieno[3,2-d]pyrimidin-4(3H)-one (Intermediate 8-6)

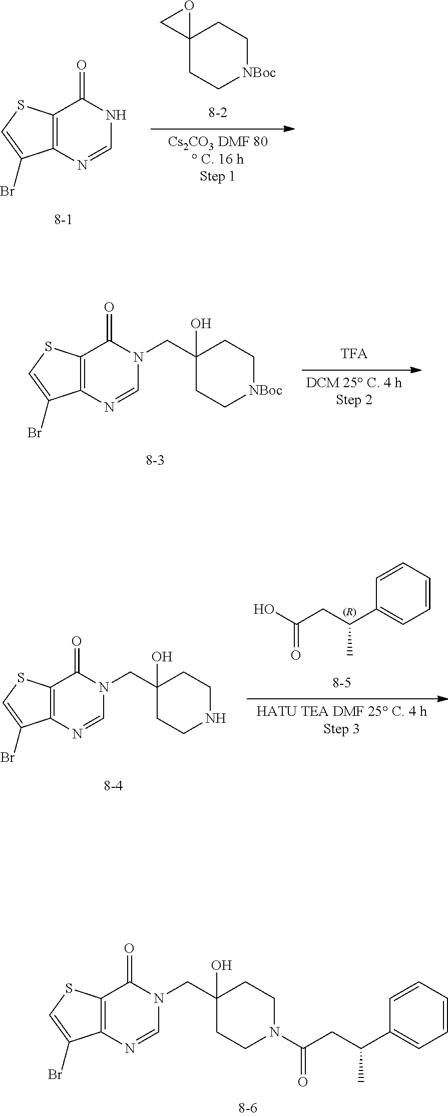

Step 1: To a solution of Intermediate 8-1 (0.5 g, 2.16 mmol, 1 eq) and Intermediate 8-2 (553.79 mg, 2.60 mmol, 1.2 eq) in DMF (5 mL) was added Cs₂CO₃ (2.12 g, 6.49 mmol, 3 eq). The mixture was stirred at 80° C. for 16 hr. LCMS showed one major peak with desired mass was detected. The reaction mixture was diluted with H₂O (20 mL) and extracted with EA 60 mL (20 mL*3). Then diluted with saturation of NaCl (10 mL) and dried over Na₂SO₄, filtered and concentrated under reduced pressure to give a residue. The residue was purified by flash silica gel chromatography (ISCO®; 20 g SepaFlash® Silica Flash Column, Eluent of 90˜100% Ethyl acetate/Petroleum ether gradient @ 40 mL/min), and the eluent was concentrated to give Intermediate 8-3 (800 mg, 1.70 mmol, 78.35% yield, 94.16% purity) was obtained as a white solid, which was confirmed by LCMS. Mass Found, LCMS: Retention time: 0.824 min, (M+H)=387.7, and LCMS: Retention time: 0.825 min, (M+H)=387.8.

Step 2: To a mixture of Intermediate 8-3 (800 mg, 1.80 mmol, 1 eq) in DCM (8 mL) and TFA (2 mL) was stirred at 25° C. for 4 hr. LCMS showed Intermediate 8-3 was consumed completely and one main peak with desired mass was detected. The reaction mixture was concentrated under reduced pressure to give a residue as crude product. The crude product Intermediate 8-4 (1.2 g, crude, TFA) as yellow solid and used into the next step without further purification. Mass Found, LCMS: Retention time: 0.326 min, (M+H)=343.8.

Step 3: To a solution of Intermediate 8-4 (1.1 g, 1.68 mmol, 70% purity, 1 eq, TFA) and Intermediate 8-5 (331.09 mg, 2.02 mmol, 217.82 μL, 1.2 eq) in DMF (11 mL) was added HATU (1.28 g, 3.36 mmol, 2 eq) and TEA (680.12 mg, 6.72 mmol, 935.52 μL, 4 eq). The mixture was stirred at 25° C. for 4 hr. LCMS showed no Intermediate 8-4 remained and 38.11% of desired compound was detected. The reaction mixture was filtered under reduced pressure to give a white solid as the product. The product Intermediate 8-6 (700 mg, 1.33 mmol, 78.99% yield, 92.99% purity) as a white solid was confirmed by LCMS, HNMR, and SFC. Mass Found, LCMS: Retention time: 0.826 min, (M+H)=489.8, and LCMS: Retention time: 0.825 min, (M+H)=489.9; SFC data, SFC: Retention time: 0.799 min; NMR Data, 1H NMR (400 MHz, DMSO-d₆) δ=8.46-8.33 (m, 2H), 7.30-7.20 (m, 4H), 7.19-7.11 (m, 1H), 4.97 (d, J=4.4 Hz, 1H), 4.12-3.98 (m, 2H), 3.96 (s, 1H), 3.71-3.59 (m, 1H), 3.27-3.09 (m, 2H), 2.91-2.79 (m, 1H), 2.66-2.53 (m, 2H), 1.57-1.24 (m, 4H), 1.20 (br d, J=6.4 Hz, 3H).

### Example 9. Synthesis of (R)-3-((4-hydroxy-1-(3-phenylbutanoyl)piperidin-4-yl)methyl)-7-(3-hydroxyprop-1-yn-1-yl)thieno[3,2-d]pyrimidin-4(3H)-one (Intermediate 9-3)

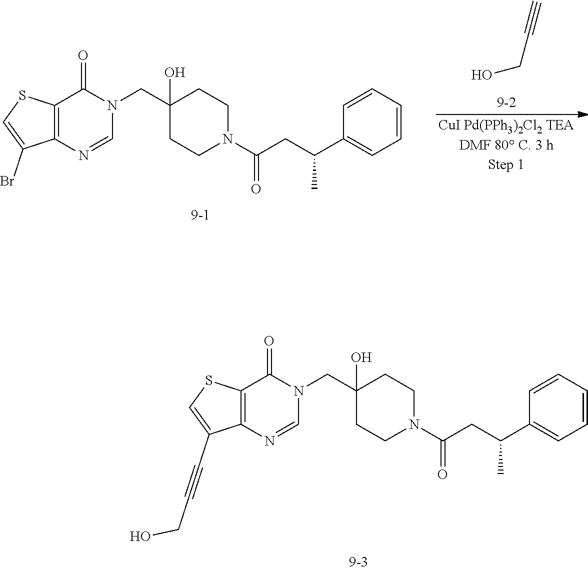

Step 1: To a solution of Intermediate 9-1 (500 mg, 1.02 mmol, 1 eq) and Intermediate 9-2 (571.59 mg, 10.20 mmol, 602.31 μL, 10 eq) in DMF (5 mL) was added TEA (309.50 mg, 3.06 mmol, 425.73 μL, 3 eq), Pd(PPh₃)₂Cl₂ (71.56 mg, 101.96 μmol, 0.1 eq) and CuI (19.42 mg, 101.96 μmol, 0.1 eq). The mixture was stirred at 80° C. under N₂ for 3 hr. LCMS showed 74.42% of desired mass was detected. The reaction was cooled to room temperature and diluted with H₂O (20 mL), extracted with ethyl acetate 90 mL (30 mL*3), The organic phase was washed with saturated aqueous NaHCO₃ (20 mL). Then dried over Na₂SO₄, filtered and concentrated under reduced pressure to give a residue. The water phase was washed with saturated aqueous NaClO (50 mL), until starch potassium iodide paper turn to blue. The residue was purified by prep-HPLC (column: Phenomenex luna C18 250*50 mm*15 μm; mobile phase: [water (0.2% FA)-ACN]; B %: 30%-50%, 10 min). The eluent was lyophilized to afford Intermediate 9-3 (270 mg, 555.47 μmol, 54.48% yield, 95.78% purity) was obtained as a yellow solid. It was confirmed by LCMS, HNMR, and SFC. Mass Found: LCMS Retention time: 0.776 min, (M+H)⁺=466.1, and Retention time: 0.776 min, (M+H)⁺=466.1; SFC data: Retention time: 1.806 min; 1H NMR (400 MHz, DMSO-d₆) δ=8.37 (s, 1H), 8.33 (d, J=10.4 Hz, 1H), 7.30-7.22 (m, 4H), 7.19-7.11 (m, 1H), 5.43-5.40 (m, 1H), 4.95 (br d, J=4.4 Hz, 1H), 4.34 (d, J=5.6 Hz, 2H), 4.12-3.98 (m, 2H), 3.97-3.92 (m, 1H), 3.72-3.58 (m, 1H), 3.24-3.09 (m, 2H), 2.92-2.79 (m, 1H), 2.65-2.53 (m, 2H), 1.59-1.23 (m, 4H), 1.22-1.16 (m, 3H).

### Example 10. Synthesis of (R)-3-((4-hydroxy-1-(3-phenylbutanoyl)piperidin-4-yl)methyl)-7-(3-hydroxypropyl)thieno[3,2-d]pyrimidin-4(3H)-one (Intermediate 10-2)

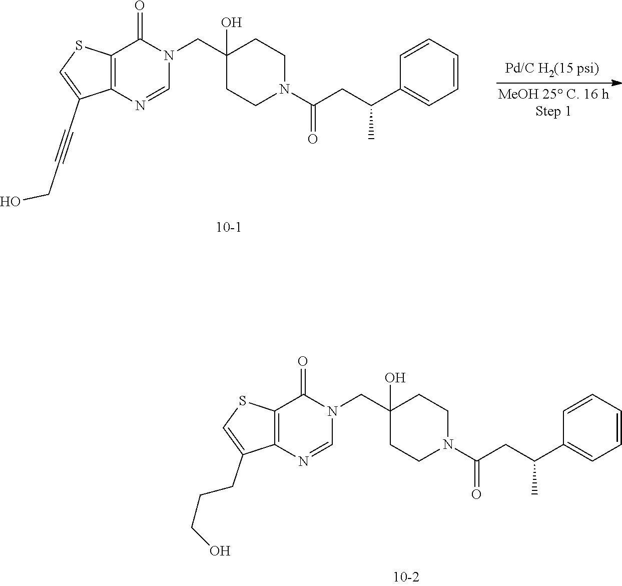 [SMILES: C[C@H](CC(=O)N1CCC(O)(Cn2cnc3c(C#CCO)csc3c2=O)CC1)c1ccccc1 ; C[C@H](CC(=O)N1CCC(O)(Cn2cnc3c(CCCO)csc3c2=O)CC1)c1ccccc1]

Step 1: To a solution of Pd/C (45.72 mg, 42.96 μmol, 10% purity, 1 eq) under N₂ atmosphere was added into a solution of Intermediate 10-1 (20 mg, 42.96 μmol, 1 eq) in MeOH (2 mL). The suspension was degassed and purged with H₂ for 3 times. The mixture was stirred under H₂ (15 Psi or atm.) at 25° C. for 16 hr. LCMS showed Intermediate 10-1 was consumed completely and one main peak with or desired mass was detected. The reaction was filtered to give a residue. The residue was purified by prep-HPLC (column: Phenomenex Synergi C18 150*25 mm*10 um; mobile phase: [water (0.225% FA)-ACN]; B %: 23%-53%, 10 min). The eluent was lyophilized to afford Intermediate 10-2 (7 mg, 14.91 μmol, 34.70% yield, 100% purity, FA) was obtained as a white solid. It was confirmed by HNMR, LCMS, and SFC. Mass Found: LCMS Retention time: 0.768 min, (M+H)⁺=470.1, and Retention time: 0.815 min, (M+H)⁺=470.1; SFC data, SFC: Retention time: 1.603 min; NMR Data, 1H NMR (400 MHz, DMSO-d₆) δ=8.30 (d, J=10.4 Hz, 1H), 7.84 (s, 1H), 7.30-7.22 (m, 4H), 7.19-7.10 (m, 1H), 4.96 (br d, J=3.2 Hz, 1H), 4.51 (br s, 1H), 4.10-3.98 (m, 2H), 3.97-3.92 (m, 1H), 3.71-3.58 (m, 1H), 3.47-3.42 (m, 2H), 3.24-3.10 (m, 2H), 2.92-2.83 (m, 1H), 2.79-2.75 (m, 2H), 2.64-2.56 (m, 2H), 1.83-1.76 (m, 2H), 1.53-1.23 (m, 4H), 1.20 (br d, J=6.8 Hz, 3H).

### Example 11. Synthesis of (R)-tert-butyl (2-(2-((3-(3-((4-hydroxy-1-(3-phenylbutanoyl)piperidin-4-yl)methyl)-4-oxo-3,4-dihydrothieno[3,2-d]pyrimidin-7-yl)prop-2-yn-1-yl)oxy)ethoxy)ethyl)carbamate (Intermediate 11-3)

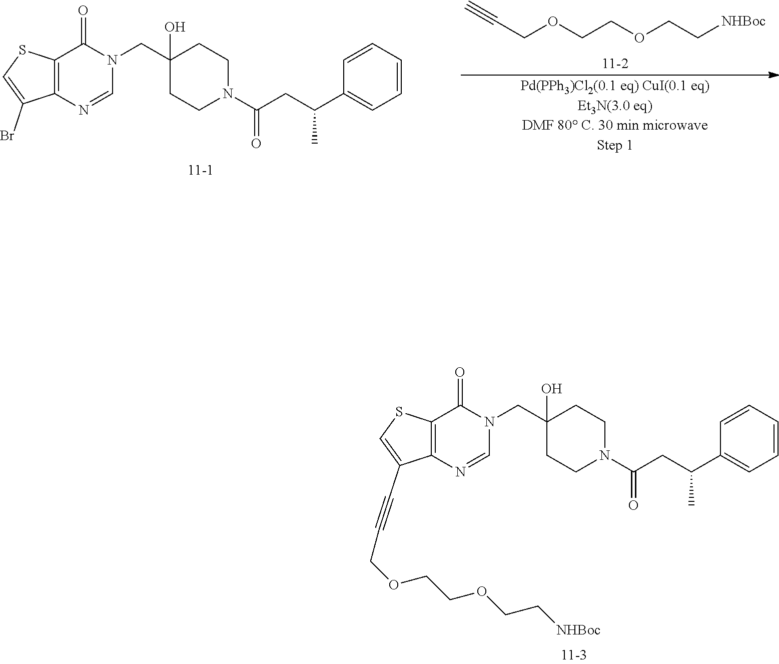 [SMILES: C[C@H](CC(=O)N1CCC(O)(Cn2cnc3c(Br)csc3c2=O)CC1)c1ccccc1 ; C#CCOCCOCCNC(=O)OC(C)(C)C ; C[C@H](CC(=O)N1CCC(O)(Cn2cnc3c(C#CCOCCOCCNC(=O)OC(C)(C)C)csc3c2=O)CC1)c1ccccc1]

Step 1: To a solution of Intermediate 11-1 (50 mg, 101.96 μmol, 1 eq), Intermediate 11-2 (124.03 mg, 509.78 μmol, 5 eq), Pd(PPh₃)₂Cl₂ (7.16 mg, 10.20 μmol, 0.1 eq), CuI (1.94 mg, 10.20 μmol, 0.1 eq) and TEA (30.95 mg, 305.87 μmol, 42.57 μL, 3 eq) were taken up into a microwave tube in DMF (0.5 mL). The sealed tube was heated at 80° C. for 30 min under microwave. LCMS showed new peaks were shown on LCMS and 30.51% of desired compound was detected. The reaction was cooled to room temperature and was diluted with H₂O (5 mL), extracted with ethyl acetate 30 mL (10 mL*3), The organic phase was washed with saturated aqueous NaHCO₃ (5 mL). Then dried over Na₂SO₄, filtered and concentrated under reduced pressure to give a residue. The residue was purified by prep-HPLC (column: Phenomenex Synergi C18 150*25 mm*10 um; mobile phase: [water (0.225% FA)-ACN]; B %: 44%-71%, 9 min), The eluent was lyophilized to afford product Intermediate 11-3 (15 mg, 21.37 μmol, 20.96% yield, 93% purity) was obtained as a yellow gum. It was confirmed by LCMS, HNMR, and SFC. Mass Found: LCMS Retention time: 0.880 min, (M+H)⁺=653.3, and Retention time: 0.947 min, (M+H)+=653.2; SFC data, SFC: Retention time: 2.090 min; NMR Data, 1H NMR (400 MHz, DMSO-d₆) δ=8.43 (s, 1H), 8.34 (d, J=10.0 Hz, 1H), 7.31-7.20 (m, 4H), 7.17-7.13 (m, 1H), 6.81-6.72 (m, 1H), 4.95 (br d, J=3.2 Hz, 1H), 4.44 (s, 2H), 4.14-3.99 (m, 2H), 3.98-3.91 (m, 1H), 3.69-3.61 (m, 3H), 3.59-3.53 (m, 2H), 3.41-3.37 (m, 2H), 3.22-3.13 (m, 2H), 3.09-3.04 (m, 2H), 2.92-2.76 (m, 1H), 2.65-2.54 (m, 2H), 1.47-1.23 (m, 13H), 1.20 (br d, J=6.0 Hz, 3H).

### Example 12. Synthesis of tert-butyl (3-(4-(4-chloro-2-(2-((2,5-dioxopyrrolidin-1-yl)methyl)thieno[3,2-b]pyridin-7-yl)-6-methylphenoxy)piperidin-1-yl)propyl)carbamate (Intermediate 12-14)

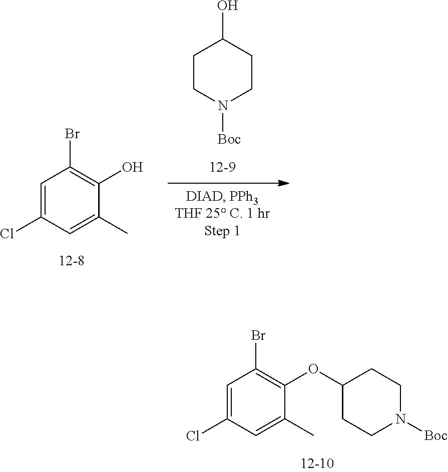

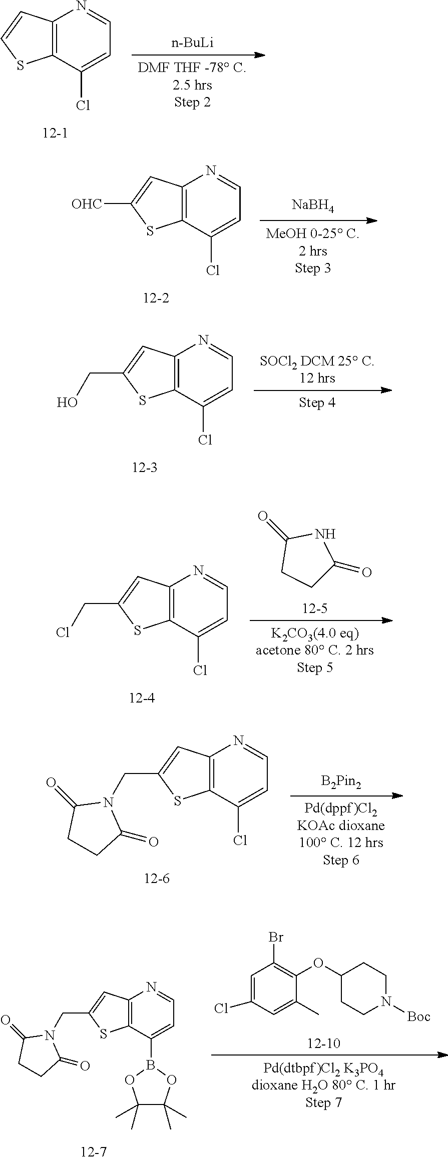

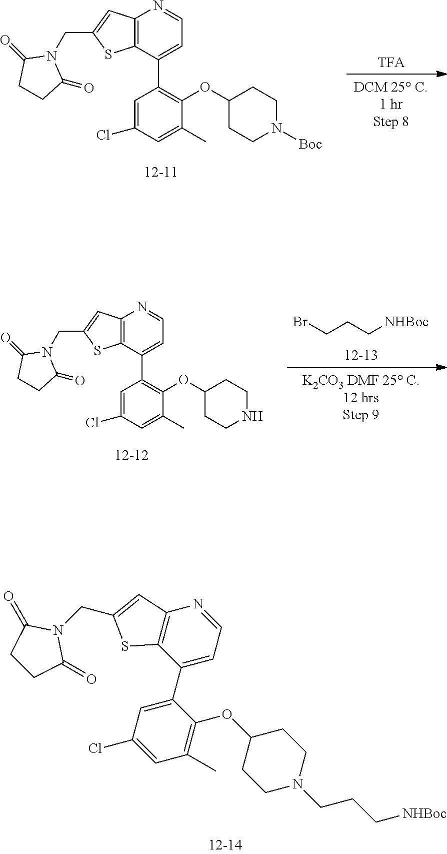

Step 1: To a solution of DIAD (4.56 g, 22.58 mmol, 4.39 mL, 1 eq) and PPh₃ (5.92 g, 22.58 mmol, 1 eq) in THE (10 mL) was added Intermediate 12-8 (5 g, 22.58 mmol, 1 eq) and Intermediate 12-9 (4.54 g, 22.58 mmol, 1 eq), then the mixture was stirred at 25° C. for 1 hr. LCMS showed desired molecular weight was detected. The mixture was diluted with DCM (7 mL) and then purified by flash silica gel chromatography (ISCO®; 80 g SepaFlash® Silica Flash Column, Eluent of 0-60% EA/PE, PE:EA=3:1, Rf=0.5) and the eluent was concentrated to give Intermediate 12-10 (3 g, 7.41 mmol, 32.83% yield) as yellow oil and confirmed by LCMS. Mass Found, LCMS: Retention time: 1.082 min, (M+H−56)=350.0, and LCMS: Retention time: 1.072 min, (M+H−56)=349.8.

Step 2: To a solution of Intermediate 12-1 (30 g, 176.85 mmol, 1 eq) in THE (300 mL) was added n-BuLi (2.5 M, 84.89 mL, 1.2 eq) at −78° C. under N₂ atmosphere and the mixture was stirred for 0.5 hr, then added DMF (71.25 g, 974.77 mmol, 75.00 mL, 5.51 eq) into the mixture and stirred 2 hrs at −78° C. LCMS showed desired molecular weight was detected. The mixture was washed with water (200 mL) and extracted with EA (300 mL*3). The combined organic layers were dried over anhydrous sodium sulfate, filtered and concentrated under reduced pressure to give crude product. The crude product was washed with MTBE (200 mL) and filtered to give a yellow solid, then concentrated to give Intermediate 12-2 (30 g, 151.79 mmol, 85.83% yield) was yellow solid and confirmed by HNMR. Mass Found, LCMS: Retention time: 0.771 min, (M+H)=198.0; NMR Data, ¹H NMR (400 MHz, DMSO-d₆) δ=10.25 (s, 1H), 8.82 (d, J=5.2 Hz, 1H), 8.64 (s, 1H), 7.81 (d, J=5.2 Hz, 1H).

Step 3: To a solution of Intermediate 12-2 (29 g, 146.73 mmol, 1 eq) in MeOH (290 mL) was added NaBH₄ (8.88 g, 234.77 mmol, 1.6 eq) at 0° C., then the mixture was stirred at 25° C. for 2 hrs. LCMS showed desired molecular weight was detected. The mixture was washed with water (300 mL) and filtered to give Intermediate 12-3 (20 g, 100.17 mmol, 68.27% yield) as white solid and confirmed by LCMS and HNMR. Mass Found, LCMS: Retention time: 0.556 min, (M+H)=199.9, and LCMS: Retention time: 0.580 min, (M+H)=199.9; NMR Data, ¹H NMR (400 MHz, Methanol-d₄) δ=8.53 (d, J=5.2 Hz, 1H), 7.46-7.38 (m, 2H), 4.95 (d, J=1.2 Hz, 2H).

Step 4: To a solution of Intermediate 12-3 (11 g, 55.09 mmol, 1 eq) in DCM (110 mL) was added SOCl₂ (19.66 g, 165.28 mmol, 11.99 mL, 3 eq) at 25° C., then the mixture was stirred at 25° C. for 12 hrs. LCMS showed desired molecular weight was detected. The mixture was concentrated under reduced pressure to give Intermediate 12-4 (12 g, crude) as white solid and used in next step directly. Mass Found, LCMS: Retention time: 0.827 min, (M+H)=217.9.

Step 5: To a solution of Intermediate 12-4 (12 g, 55.02 mmol, 1 eq) in acetone (250 mL) was added Intermediate 12-5 (11.99 g, 121.04 mmol, 2.2 eq) and K₂CO₃ (30.42 g, 220.08 mmol, 4 eq), then the mixture was stirred at 80° C. for 2 hrs. LCMS showed desired molecular weight was detected. The mixture was washed with water (200 mL) and extracted with DCM (200 mL*3). The combined organic layers were dried over anhydrous sodium sulfate and concentrated under reduced pressure to give Intermediate 12-6 (15 g, 53.43 mmol, 97.11% yield, N/A purity) was brown solid and confirmed by LCMS. Mass Found, LCMS: Retention time: 0.713 min, (M+H)=281.1, and LCMS: Retention time: 0.695 min, (M+H)=281.0.

Step 6: To a solution of Intermediate 12-6 (1 g, 3.56 mmol, 1 eq) and B₂Pin₂ (2.71 g, 10.69 mmol, 3 eq) in dioxane (10 mL) was added KOAc (1.05 g, 10.69 mmol, 3 eq) and Pd(dppf)Cl₂ (521.29 mg, 712.43 μmol, 0.2 eq), then the mixture was stirred at 100° C. for 12 hrs under N₂ atmosphere. LCMS showed desired molecular weight was detected. The mixture was filtered, the organic phase was concentrated under reduced pressure to give Intermediate 12-7 (1.3 g, 3.49 mmol, 98.04% yield) as brown solid.

Step 7: To a solution of Intermediate 12-7 (1 g, 2.69 mmol, 1 eq) and Intermediate 12-10 (1.09 g, 2.69 mmol, 1 eq) in dioxane (10 mL) and H₂O (2 mL) was added Pd(dtbpf)Cl₂ (175.08 mg, 268.64 μmol, 0.1 eq) and K₃PO₄ (1.71 g, 8.06 mmol, 3 eq), then the mixture was stirred at 80° C. for 1 hr under N₂ atmosphere. LCMS showed desired molecular weight was detected. The mixture was filtered, the organic phase was concentrated under reduced pressure to give crude product. The residue was purified by column chromatography (SiO₂, Petroleum ether/Ethyl acetate=100/0 to 0/100, PE:EA=1:1 Rt=0.2) and concentrated to give Intermediate 12-11 (0.8 g, 1.40 mmol, 52.24% yield) as brown oil and confirmed by LCMS. Mass Found, LCMS: Retention time: 0.919 min, (M+H)=570.1, and LCMS: Retention time: 0.954 min, (M+H)=570.2.

Step 8: To a solution of Intermediate 12-11 (0.7 g, 1.23 mmol, 1 eq) in DCM (7 mL) was added TFA (2.16 g, 18.91 mmol, 1.40 mL, 15.40 eq) at 25° C., then the mixture was stirred at 25° C. for 1 hr. LCMS showed desired molecular weight was detected. The mixture was concentrated to give Intermediate 12-12 (0.57 g, 1.21 mmol, 98.77% yield) as brown oil and confirmed by HPLC. Mass Found, LCMS: Retention time: 0.725 min, (M+H)=470.1.

Step 9: To a solution of Intermediate 12-12 (0.57 g, 1.21 mmol, 1 eq) and Intermediate 12-13 (404.31 mg, 1.70 mmol, 1.4 eq) in DMF (6 mL) was added K₂CO₃ (670.47 mg, 4.85 mmol, 4 eq) at 25° C., then the mixture was stirred at 25° C. for 12 hrs. LCMS showed desired molecular weight was detected. The mixture was filtered and the filter liquor was used purification. The residue was purified by prep-HPLC (column: Welch Ultimate XB-CN 250*50*10 um; mobile phase: [Hexane-IPA]; B %: 25%-65%, 15 min) and lyophilized to give Intermediate 12-14 (0.28 g, 415.91 μmol, 34.29% yield, 100% purity, FA) as yellow gum and confirmed by LCMS and HNMR. Mass Found, LCMS: Retention time: 0.781 min, (M+H)=627.2, and LCMS: Retention time: 0.767 min, (M+H)=627.3; NMR Data, ¹H NMR (400 MHz, DMSO+D₂O) δ=8.67 (d, J=4.4 Hz, 1H), 8.30 (d, J=3.6 Hz, 1H), 7.51-7.43 (m, 2H), 7.36 (d, J=4.8 Hz, 1H), 7.29 (d, J=2.0 Hz, 1H), 4.84 (s, 2H), 3.54-3.52 (m, 1H), 2.82 t, J=6.4 Hz, 2H), 2.68 (s, 4H), 2.42-2.18 (m, 7H), 2.13-1.93 (m, 2H), 1.46-1.27 (m, 15H).

### Example 13. 1 Synthesis of tert-butyl (3-(4-(4-chloro-2-(2-((2,5-dioxopyrrolidin-1-yl)methyl)thieno[3,2-b]pyridin-7-yl)-6-methylphenoxy)piperidin-1-yl)-3-oxopropyl)carbamate (Intermediate 13-3)

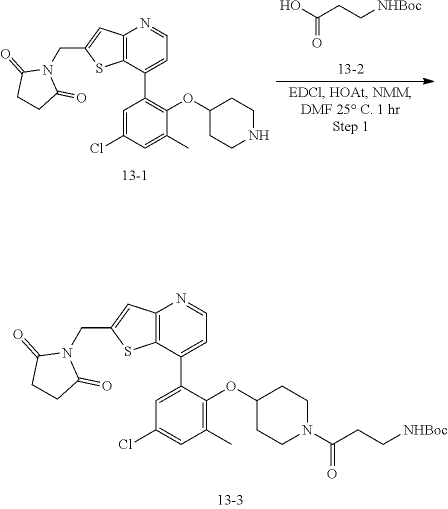 [SMILES: Cc1cc(Cl)cc(-c2ccnc3cc(CN4C(=O)CCC4=O)sc23)c1OC1CCNCC1 ; CC(C)(C)OC(=O)NCCC(=O)O ; Cc1cc(Cl)cc(-c2ccnc3cc(CN4C(=O)CCC4=O)sc23)c1OC1CCN(C(=O)CCNC(=O)OC(C)(C)C)CC1]

Step 1: To a solution of Intermediate 13-1 (0.02 g, 42.55 μmol, 1 eq) and Intermediate 13-2 (12.08 mg, 63.83 μmol, 1.5 eq) in DMF (0.2 mL) was added EDCI (16.32 mg, 85.11 μmol, 2 eq) and NMM (21.52 mg, 212.77 μmol, 23.39 μL, 5 eq) and HOAt (2.90 mg, 21.28 μmol, 2.98 μL, 0.5 eq) at 25° C., then the mixture was stirred at 25° C. for 1 hr. LCMS showed desired molecular weight was detected. The reaction was poured into water (0.2 mL) and extracted with EA 1 mL (0.3 mL*3). The organic layers were dried over anhydrous Na₂SO₄, filtered and concentrated to give a residue. The residue was purified by prep-HPLC (column: Phenomenex Synergi C18 150*25 mm*10 um; mobile phase: [water (0.225% FA)-ACN]; B %: 48%-78%, 10 min) and lyophilized to give Intermediate 13-3 (9.95 mg, 15.52 μmol, 49.75% yield, 100% purity) as yellow gum and confirmed by LCMS and HNMR. Mass Found, LCMS: Retention time: 0.868 min, (M+H)=641.2, and LCMS: Retention time: 0.933 min, (M+H)=641.2; NMR Data, ¹H NMR (400 MHz, Chloroform-d) δ=8.72 (d, J=4.8 Hz, 1H), 7.60 (s, 1H), 7.31 (d, J=2.8 Hz, 1H), 7.29 (s, 1H), 7.27 (s, 1H), 5.23 (s, 1H), 4.98 (s, 2H), 3.72-3.62 (m, 1H), 3.60-3.52 (m, 1H), 3.38-3.33 (m, 2H), 3.31-3.22 (m, 1H), 2.92-2.81 (m, 2H), 2.79 (s, 4H), 2.41-2.34 (m, 5H), 1.46 (s, 2H), 1.43 (s, 9H), 1.37-1.30 (m, 2H).

### Example 14. Synthesis of tert-butyl (3-(6′-amino-4′-ethyl-5′-(4-hydroxyphenyl)-[3,3′-bipyridine]-6-carboxamido)propyl)carbamate (Intermediate 14-9)

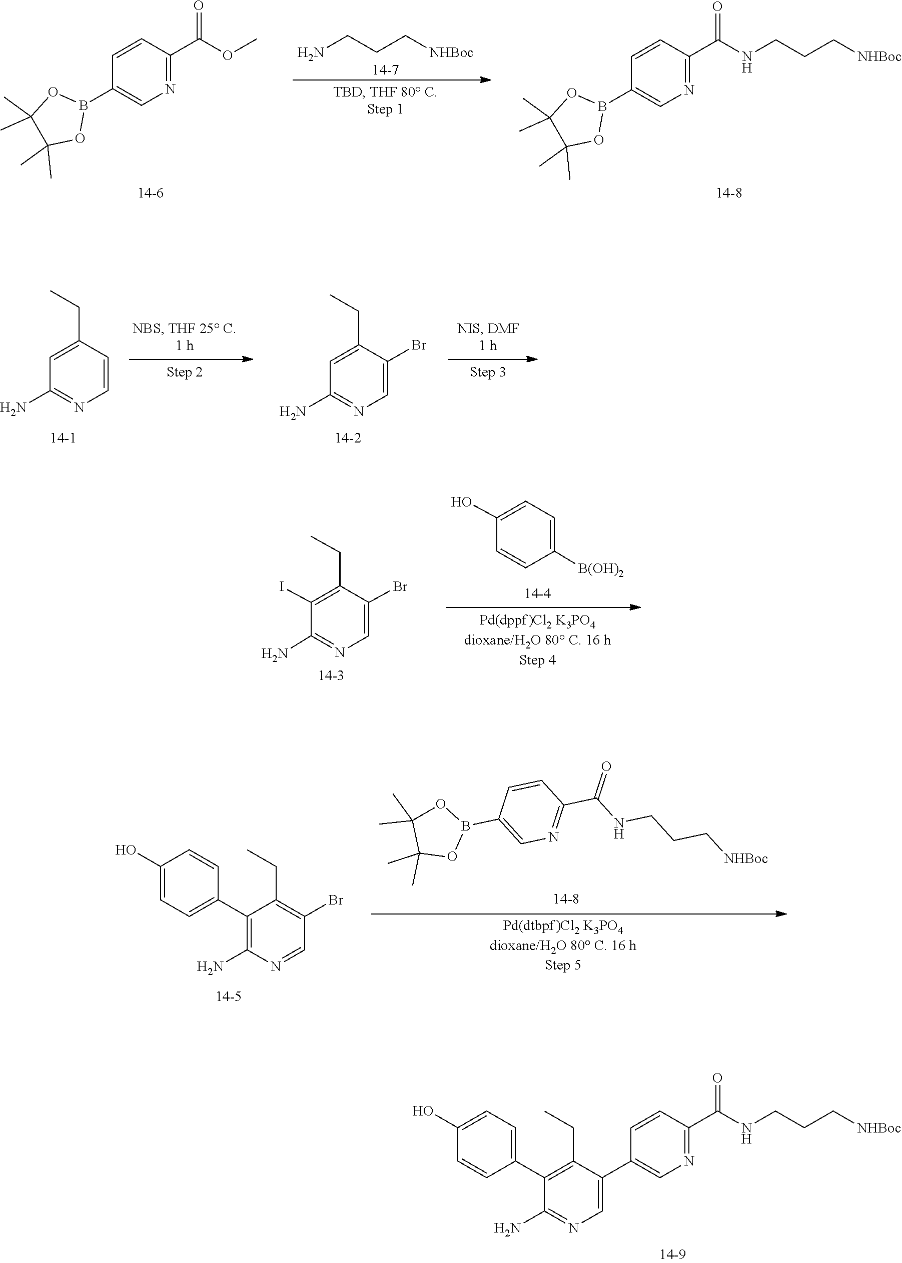

Step 1: To a solution of Intermediate 14-6 (2.5 g, 9.50 mmol, 1 eq) and Intermediate 14-7 (2.48 g, 14.25 mmol, 2.49 mL, 1.5 eq) in THF (25 mL) was added TBD (1.32 g, 9.50 mmol, 1.0 eq) at 25° C. Then the mixture was stirred for 16 h at 80° C. LCMS showed desired MW was detected. The reaction mixture was washed with H₂O (25 mL) and extracted with EA 10 mL (25 mL*2). The combined organic layers were concentrated under reduced pressure to give a residue. The crude product was purified by Prep-HPLC (column: Welch Ultimate XB-CN 250*50*10 um; mobile phase: [Hexane-EtOH (0.1% NH₃·H₂O]; B %: 1%-35%, 15 min) and lyophilized to give desired product Intermediate 14-8 (3 g, 6.66 mmol, 70.11% yield, 90% purity) as brown solid which was confirmed by HNMR. Mass found: LCMS Retention time: 0.697 min, (M+H)=324.2; NMR data: ¹HNMR 400 MHz, Chloroform-d) δ=8.27 (br d, J=9.6 Hz, 1H), 8.23-8.21 (m, 1H), 8.16-8.13 (m, 1H), 3.56-3.53 (m, 2H), 3.20-3.17 (m, 2H), 1.79-1.77 (m, 2H), 1.44 (s, 9H), 1.37 (s, 13H).

Step 2: To a solution of Intermediate 14-1 (2.0 g, 16.37 mmol, 1 eq) in THF (40 mL) was added NBS (2.91 g, 16.37 mmol, 1.0 eq) at 0° C., then the mixture was stirred for 30 min. TLC (PE/EA=3:1, Rf=0.5) showed a new spot was detected. The mixture was washed with water (50 ml) and extracted with EA (50 ML*3), the organic layer was separated and concentrated to give crude product. The residue was purified by column chromatography (SiO₂, Petroleum ether/Ethyl acetate=100/1 to 50/1) to give Intermediate 14-2 (3.0 g, 14.92 mmol, 91.14% yield) as brown solid which was confirmed by HNMR. NMR Data: 1HNMR (400 MHz, Chloroform-d) δ=8.00 (s, 1H), 6.32 (s, 1H), 4.19 (br s, 2H), 2.57-2.51 (m, 2H), 1.14-1.10 (m, 3H).

Step 3: To a solution of Intermediate 14-2 (1 g, 4.97 mmol, 1 eq) and TFA (680.52 mg, 5.97 mmol, 441.90 μL, 1.2 eq) in DMF (20 mL) was added portion-wise NIS (1.68 g, 7.46 mmol, 1.5 eq) at 0° C. The reaction mixture was stirred at 55° C. for 2 h. LCMS showed desired mass was detected. The reaction mixture was quenched with ice water (30 mL) and sodium thiosulphate solution (10 mL), and then precipitated by adding saturated NaHCO₃ solution 5 mL, stirring for 10 min. The solid compound was collected by filtration to give a residue. The residue was purified by flash silica gel chromatography (ISCO®; 40 g SepaFlash® Silica Flash Column, Eluent of 0˜10% Ethyl acetate/Petroleum ether gradient @ 30 mL/min). Intermediate 14-3 (1.4 g, 3.97 mmol, 79.92% yield, 92.828% purity) was obtained as a yellow solid, which was confirmed by LCMS Retention time: 0.817 min, (M+H)=326.8.

Step 4: To a solution of Intermediate 14-3 (1.3 g, 3.98 mmol, 1 eq) and Intermediate 14-4 (658.09 mg, 4.77 mmol, 1.2 eq) in dioxane (13 mL) and H₂O (3.25 mL) was added K₃PO₄ (1.69 g, 7.95 mmol, 2 eq) and cyclopentyl(diphenyl)phosphane;dichloropalladium;iron (290.93 mg, 397.60 μmol, 0.1 eq). The reaction mixture was stirred at 80° C. for 6 hrs. LC-MS showed desired mass was detected. The reaction mixture was quenched by addition water 20 mL, extracted with EA 30 mL (10 mL*3). The combined organic layers were dried over Na₂SO₄, filtered and concentrated under reduced pressure to give a residue. The residue was purified by flash silica gel chromatography (ISCO®; 80 g SepaFlash® Silica Flash Column, Eluent of 50-100% Ethyl acetate/Petroleum ether gradient @ 50 mL/min). Intermediate 14-5 (0.7 g, 2.39 mmol, 60.05% yield, N/A purity) was obtained as a yellow solid which was confirmed by 1H NMR. Mass Found: LCMS Retention time: 0.731 min, (M+H)=295.0; NMR Data: 1H NMR (400 MHz, Chloroform-d) δ=8.15 (s, 1H), 8.05 (s, 1H), 7.50-7.46 (m, 1H), 7.36-7.31 (m, 1H), 7.13-7.06 (m, 2H), 7.05-6.97 (m, 2H), 5.23-5.03 (m, 2H), 4.53 (br s, 1H), 2.55-2.49 (m, 2H), 1.30-1.21 (m, 3H).

Step 5: To a solution of Intermediate 14-5 (0.4 g, 1.36 mmol, 1 eq) and Intermediate 14-8 (663.61 mg, 1.64 mmol, 1.2 eq) in dioxane (4 mL) and H₂O (1 mL) was added K₃PO₄ (579.25 mg, 2.73 mmol, 2 eq) and di-tert-butyl(cyclopentyl)phosphane;dichloropalladium;iron (88.93 mg, 136.44 μmol, 0.1 eq). The reaction mixture was stirred at 80° C. for 6 hrs. Extra Intermediate 14-8 (663.61 mg, 1.64 mmol, 1.2 eq), di-tertbutyl(cyclopentyl)phosphane;dichloropalladium;iron (88.93 mg, 136.44 μmol, 0.1 eq) and K₃PO₄ (579.25 mg, 2.73 mmol, 2 eq) were added into the reaction mixture. The mixture was stirred at 80° C. for 12 hrs. LCMS showed desired mass was detected. The reaction mixture was diluted with water 15 mL and extracted with EA 30 mL (10 mL*3). The combined organic layers were dried over Na₂SO₄, filtered and concentrated under reduced pressure to give a residue. The residue was purified by prep-HPLC (FA condition; column: Phenomenex Synergi C18 150*25 mm*10 um; mobile phase: [water (0.225% FA)-ACN]; B %: 8%-41%, 11 min). Intermediate 14-9 (0.383 g, 779.12 μmol, 57.10% yield, 100% purity) was obtained as a yellow solid. Mass Found: LCMS Retention time: 0.729 min, (M+H)=492.3, and Retention time: 0.805 min, (M+H)=492.1; NMR Data: ¹H NMR (400 MHz, Methanol-d₄) δ=8.62 (d, J=1.6 Hz, 1H), 8.15 (d, J=8.0 Hz, 1H), 7.98-7.94 (m, 1H), 7.75 (s, 1H), 7.15-7.10 (m, 2H), 7.00-6.91 (m, 2H), 3.50-3.46 (m, 2H), 3.17-3.13 (m, 2H), 2.42-2.36 (m, 2H), 1.82-1.75 (m, 2H), 1.43 (s, 9H), 0.73-0.68 (m, 3H).

### Example 15. Synthesis of 6′-amino-N-(2-(2-(2-(2-azidoethoxy)ethoxy)ethoxy)ethyl)-4′-ethyl-5′-(4-hydroxyphenyl)-[3,3′-bipyridine]-6-carboxamide (Intermediate 15-9)

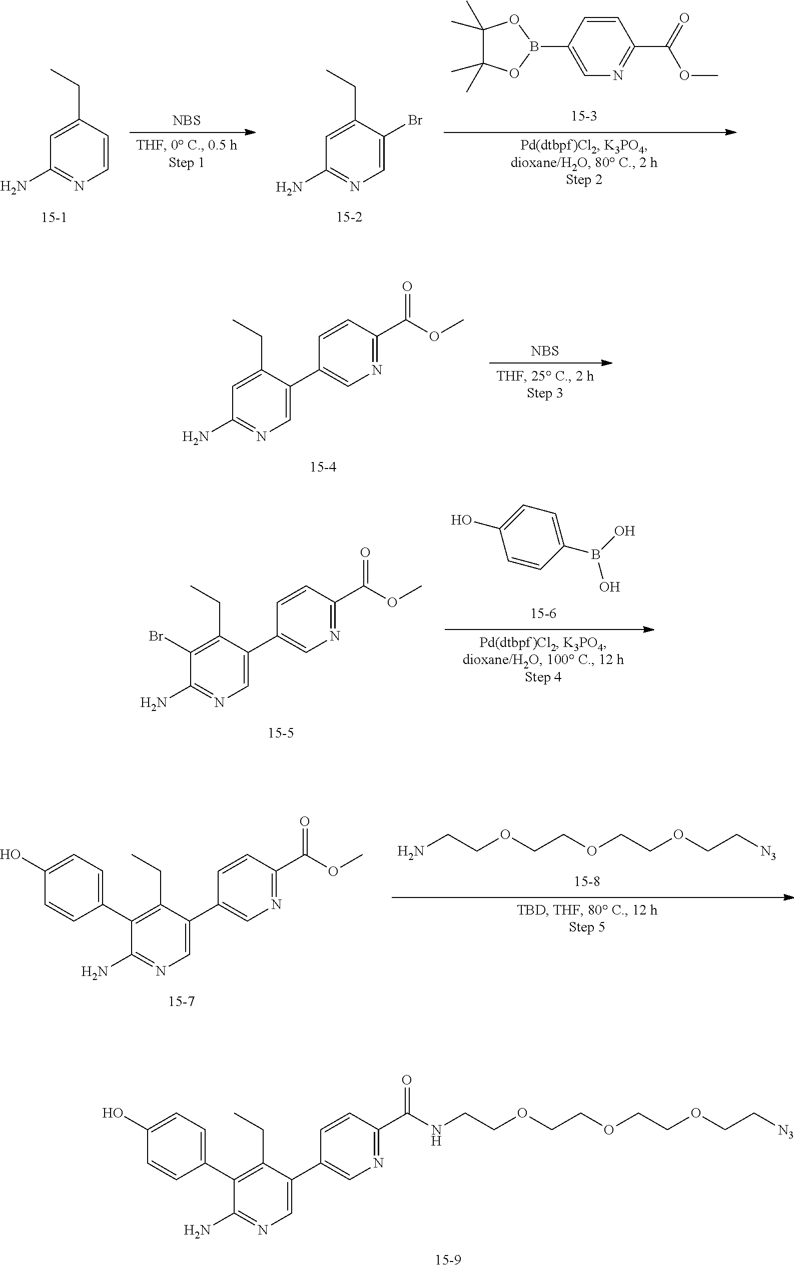

Step 1: To a solution of Intermediate 15-1 (2.0 g, 16.37 mmol, 1 eq) in THF (40 mL) was added NBS (2.91 g, 16.37 mmol, 1.0 eq) at 0° C., then the mixture was stirred for 30 min. TLC (PE/EA=3:1, Rf=0.5) showed a new spot was detected. The mixture was washed with water (50 ml) and extracted with EA (50 mL*3), the organic layer was separated and concentrated to give crude product. The crude product was purified by column chromatography (SiO₂, Petroleum ether/Ethyl acetate=100/1 to 50/1) to give Intermediate 15-2 (3.0 g, 14.92 mmol, 91.14% yield) as brown solid which was confirmed by ¹HNMR. NMR Data, ¹H NMR (400 MHz, Chloroform-d) δ=8.00 (s, 1H), 6.32 (s, 1H), 4.19 (br s, 2H), 2.57-2.51 (m, 2H), 1.14-1.10 (m, 3H).

Step 2: To a solution of Intermediate 15-2 (1.0 g, 4.97 mmol, 1 eq) and Intermediate 15-3 (1.57 g, 5.97 mmol, 1.2 eq) in dioxane (8 mL) and H₂O (2 mL) was added K₃PO₄ (3.17 g, 14.92 mmol, 3.0 eq) and Pd(dtbpf)Cl₂ (162.07 mg, 248.68 μmol, 0.05 eq) at 25° C. Then the mixture was stirred for 2 hrs at 80° C. LCMS showed desired MW was detected. The mixture was washed with water (5 ml) and extracted with EA (10 mL*2), the organic layer was separated and concentrated to give crude product. The crude product was purified by reverse-phase (0.1% FA) and the eluent was concentrated to give Intermediate 15-4 (1.0 g, 3.89 mmol, 78.15% yield) as brown solid which was confirmed by HNMR. Mass Found, LCMS: Retention time: 0.557 min, (M+H)=258.1; NMR Data, ¹H NMR (400 MHz, Chloroform-d) δ=8.69 (d, J=1.8 Hz, 1H), 8.20 (d, J=8.0 Hz, 1H), 7.86 (s, 1H), 7.78-7.76 (m, 1H), 6.50 (s, 1H), 4.05 (s, 3H), 2.55-2.49 (m, 2H), 1.11-1.08 (m, 3H).

Step 3: To a mixture of Intermediate 15-4 (513 mg, 1.99 mmol, 1 eq) in THF (5 mL) was added NBS (354.88 mg, 1.99 mmol, 1 eq) and then the mixture was stirred at 25° C. for 2 hrs. LCMS showed Reactant 1 was consumed completely and one major peak with desired mass was detected. The mixture was poured into water 5 mL and extracted with EA 15 mL (5 mL*3). The combined organic layers were dried over anhydrous Na₂SO₄, filtered and concentrated to give a residue. The residue was purified by flash silica gel chromatography (ISCO®; 40 g SepaFlash® Silica Flash Column, Eluent of 0˜100% Ethyl acetate/Petroleum ether gradient @ 30 mL/min) and concentrated to afford Intermediate 15-5 (290 mg, 845.11 μmol, 42.39% yield, 97.97% purity) was obtained as a yellow solid, which was confirmed by LCMS and ¹HNMR. Mass Found, LCMS: Retention time: 0.703 min, (M+H)=335.9, and LCMS: Retention time: 0.754 min, (M+H)=336.0; NMR Data, ¹H NMR (400 MHz, Chloroform-d) δ=8.74-8.67 (m, 1H), 8.21 (d, J=8.0 Hz, 1H), 7.83 (s, 1H), 7.80-7.74 (m, 1H), 5.16 (br s, 2H), 4.05 (d, J=0.8 Hz, 3H), 2.71-2.64 (m, 2H), 1.10-1.06 (m, 3H).

Step 4: To a mixture of Intermediate 15-5 (290 mg, 862.62 μmol, 1 eq), Intermediate 15-6 (356.94 mg, 2.59 mmol, 3 eq) and K₃PO₄ (549.32 mg, 2.59 mmol, 3 eq) in dioxane (3 mL) and H₂O (0.6 mL) was added Pd(dtbpf)Cl₂ (56.22 mg, 86.26 μmol, 0.1 eq) and then the mixture was stirred at 100° C. for 12 hrs under N₂. LCMS showed 14% of desired molecular weight was detected. The reaction was poured into water 5 mL and extracted with EA 15 mL (5 mL*3). The organic layers were dried over anhydrous Na₂SO₄, filtered and concentrated to give a residue. The residue was purified by reversed phase (0.1% FA condition) and the eluent was concentrated to give the residue. The residue was repurified by reversed phase (0.1% FA condition) and the eluent was concentrated to afford Intermediate 15-7 (17 mg, 48.66 μmol, 5.64% yield) was obtained as a yellow solid, which was confirmed by HNMR and LCMS. Mass Found, LCMS: Retention time: 0.684 min, (M+H)=350.1, and LCMS: Retention time: 0.657 min, (M+H)=350.1; NMR Data, ¹H NMR (400 MHz, Chloroform-d) δ=8.75 (d, J=1.2 Hz, 1H), 8.37-8.30 (m, 1H), 8.25 (d, J=8.0 Hz, 1H), 7.90-7.82 (m, 1H), 7.71 (s, 1H), 7.17-7.08 (m, 2H), 7.08-6.99 (m, 2H), 6.45-6.05 (m, 2H), 4.06 (s, 3H), 2.50-2.26 (m, 2H), 0.74-0.69 (m, 3H).

Step 5: To a mixture of Intermediate 15-7 (17 mg, 48.66 μmol, 1 eq) and Intermediate 15-8 (23.36 mg, 107.05 μmol, 2.2 eq) in THE (0.5 mL) was added TBD (6.77 mg, 48.66 μmol, 1 eq) and then the mixture was stirred at 80° C. for 12 hrs. LCMS showed desired molecular weight was detected. The mixture was concentrated under reduced pressure to give a residue. The residue was purified by prep-HPLC (column: Phenomenex luna C18 150*25 mm*10 μm; mobile phase: [water (0.225% FA)-ACN]; B %: 8%-38%, 10 min) and lyophilized to afford Intermediate 15-9 (8.14 mg, 15.13 μmol, 31.09% yield, 99.551% purity) as obtained as yellow gum which was confirmed by HNMR and LCMS. Mass Found, LCMS: Retention time: 0.710 min, (M+H)=536.2, and LCMS: Retention time: 0.809 min, (M+H)=536.2; NMR Data, ¹H NMR (400 MHz, Chloroform-d) δ=8.54 (d, J=1.6 Hz, 1H), 8.45-8.32 (m, 1H), 8.26 (d, J=8.0 Hz, 1H), 7.89-7.76 (m, 2H), 7.16 (d, J=8.4 Hz, 2H), 7.01 (d, J=8.4 Hz, 2H), 4.77 (br s, 2H), 3.76-3.65 (m, 14H), 3.40-3.37 (m, 2H), 2.38-2.31 (m, 2H), 0.73-0.68 (m, 3H).

### Example 16. Synthesis of 7-bromo-3-((4-hydroxypiperidin-4-yl)methyl)quinazolin-4(3H)-one (Intermediate 16-5)

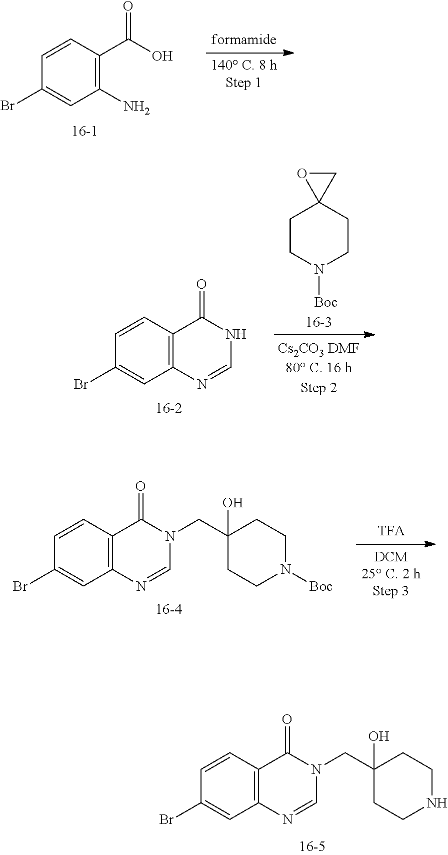 [SMILES: Nc1cc(Br)ccc1C(=O)O ; O=c1[nH]cnc2cc(Br)ccc12 ; CC(C)(C)OC(=O)N1CCC2(CC1)CO2 ; CC(C)(C)OC(=O)N1CCC(O)(Cn2cnc3cc(Br)ccc3c2=O)CC1 ; O=c1c2ccc(Br)cc2ncn1CC1(O)CCNCC1]

Step 1: To a mixture of Intermediate 16-1 (2.0 g, 9.26 mmol, 1 eq) in formamide (1.5 mL) was stirred at 140° C. for 8 hr. LCMS showed Intermediate 16-1 was consumed completely and one major peak with desired mass was detected. The reaction mixture was diluted with H₂O (10 mL) and the mixture was filtered and concentrated to give a brown solid, which was the crude product Intermediate 16-2 (1.6 g, 7.04 mmol, 76.09% yield, 99.08% purity as a brown solid, it was used into the next step without further purification. It was confirmed by LCMS and HNMR. Mass Found, LCMS: Retention time: 0.645 min, (M+H)=224.9, and LCMS: Retention time: 0.707 min, (M+H)=225.1; NMR Data, 1H NMR (400 MHz, DMSO-d₆) δ=12.38 (br s, 1H), 8.13 (s, 1H), 8.02 (d, J=8.4 Hz, 1H), 7.86 (d, J=2.0 Hz, 1H), 7.68-7.65 (m, 1H).

Step 2: To a solution of Intermediate 16-2 (1.5 g, 6.67 mmol, 1 eq) and tert-butyl 1-oxa-6-azaspiro[2.5]octane-6-carboxylate (1.71 g, 8.00 mmol, 1.2 eq) in DMF (14 mL) was added Cs₂CO₃ (6.52 g, 20.00 mmol, 3 eq). The mixture was stirred at 80° C. for 16 hr. LCMS showed Intermediate 16-2 was consumed completely and one major peak with desired mass was detected. The reaction mixture was diluted with H₂O (10 mL) and extracted with EA 60 mL (20 mL*3). Then dried over Na₂SO₄, filtered and concentrated under reduced pressure to give a residue. The residue was purified by flash silica gel chromatography (ISCO®; 40 g SepaFlash® Silica Flash Column, Eluent of 60˜90% Ethyl acetate/Petroleum ether gradient @ 40 mL/min). The eluent was concentrated to give Intermediate 16-4 (2.7 g, 5.58 mmol, 83.72% yield, 90.59% purity) was obtained as a white solid, which was confirmed by LCMS, and HNMR. Mass Found, LCMS: Retention time: 0.873 min, (M+H−56)=382.0, and LCMS: Retention time: 0.871 min, (M+H−56)=382.0; NMR Data, 1H NMR (400 MHz, DMSO-d₆) δ=8.27 (s, 1H), 8.07 (d, J=8.4 Hz, 1H), 7.90 (d, J=2.0 Hz, 1H), 7.72-7.69 (m, 1H), 4.92 (s, 1H), 4.01-3.95 (m, 2H), 3.65 (br d, J=12.8 Hz, 2H), 3.05 (br d, J=7.6 Hz, 2H), 1.47-1.43 (m, 2H), 1.42-1.31 (m, 11H).

Step 3: To a solution of Intermediate 16-4 (2.7 g, 6.16 mmol, 1 eq) in DCM (20 mL) was added TFA (7 mL), then the mixture was stirred at 25° C. for 4 hr. LCMS showed Intermediate 16-4 was consumed completely and one major peak with desired mass was detected. The reaction mixture was concentrated under reduced pressure to give a residue. The residue was purified by prep-HPLC (column: Welch Ultimate XB-CN 250*70*10 um; mobile phase: [Hexane-EtOH (0.1% NH3·H2O]; B %: 40%-80%, 15 min). The eluent was concentrated to afford Intermediate 16-5 (3.5 g, crude, TFA) was obtained as a yellow solid, which was confirmed by LCMS, HNMR, and FNMR. Mass Found, LCMS: Retention time: 0.561 min, (M+H)=337.8, and LCMS: Retention time: 0.565 min, (M+H)=338.0; NMR Data, 1H NMR (400 MHz, DMSO-d₆) δ=8.29 (s, 1H), 8.10-8.01 (m, 1H), 7.86 (d, J=1.6 Hz, 1H), 7.67-7.65 (m, 1H), 5.39 (br s, 1H), 4.04 (s, 2H), 3.20-3.11 (m, 2H), 3.10-2.96 (m, 2H), 1.86-1.70 (m, 2H), 1.59 (br d, J=14.0 Hz, 2H); 19F NMR (377 MHz, DMSO-d6).

### Example 17. Synthesis of (R)-7-bromo-3-((4-hydroxy-1-(3-phenylbutanoyl)piperidin-4-yl)methyl)quinazolin-4(3H)-one (Intermediate 17-3)

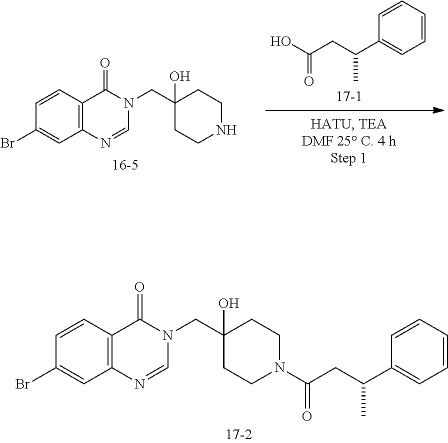 [SMILES: O=c1c2ccc(Br)cc2ncn1CC1(O)CCNCC1 ; C[C@H](CC(=O)O)c1ccccc1 ; C[C@H](CC(=O)N1CCC(O)(Cn2cnc3cc(Br)ccc3c2=O)CC1)c1ccccc1]

Step 1: To a solution of Intermediate 16-5 (2.5 g, 5.53 mmol, 1 eq, TFA) and 3-phenylbutanoic acid (1.09 g, 6.63 mmol, 716.64 μL, 1.2 eq) in DMF (25 mL) was added HATU (4.20 g, 11.06 mmol, 2 eq) and TEA (1.68 g, 16.58 mmol, 2.31 mL, 3 eq). The mixture was stirred at 25° C. for 4 hr. LCMS showed one main peak with desired mass was detected. The reaction mixture was diluted with H₂O (20 mL) and extracted with EA 60 mL (20 mL*3). Then diluted with saturation of NaCl (10 mL) and dried over Na₂SO₄, filtered and concentrated under reduced pressure to give a residue. The residue was purified by prep-HPLC (column: Phenomenex luna C18 250*50 mm*15 um; mobile phase: [water (0.2% FA)-ACN]; B %: 45%-65%, 10 min), the eluent was lyophilized to afford Intermediate 17-2 (1.0 g, 2.06 mmol, 37.34% yield, 100% purity) was obtained as a yellow solid, it was confirmed by LCMS, SFC, and HNMR. Mass Found: LCMS Retention time: 0.861 min, (M+H)⁺=483.8, and Retention time: 0.909 min, (M+H+2)⁺=485.9; SFC data, SFC: Retention time: 1.117 min; NMR Data, 1H NMR (400 MHz, DMSO-d₆) δ=8.26 (br d, J=10.4 Hz, 1H), 8.14-8.00 (m, 1H), 7.90 (s, 1H), 7.72-7.69 (m, 1H), 7.42-7.19 (m, 4H), 7.19-7.05 (m, 1H), 4.94 (br s, 1H), 4.19-3.94 (m, 2H), 3.93-3.82 (m, 1H), 3.73-3.53 (m, 1H), 3.23-3.05 (m, 2H), 2.96-2.72 (m, 1H), 2.68-2.55 (m, 2H), 1.59-1.25 (m, 4H), 1.24-1.13 (m, 3H).

### Example 18. Synthesis of (R)-tert-butyl (3-((2-((3-((4-hydroxy-1-(3-phenylbutanoyl)piperidin-4-yl)methyl)-4-oxo-3,4-dihydroquinazolin-7-yl)amino)ethyl)(methyl)amino)propyl)carbamate (Intermediate 18-6)

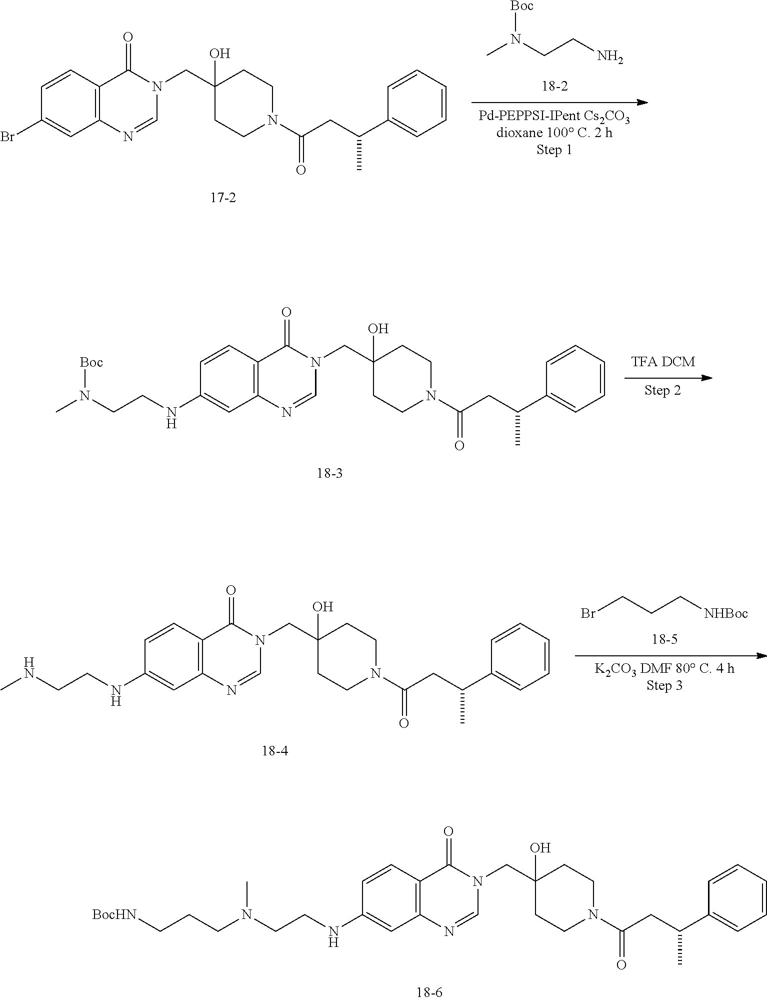

Step 1: To a solution of Intermediate 17-2 (100 mg, 206.45 μmol, 1 eq) and Intermediate 3-2 (107.91 mg, 619.34 μmol, 110.68 μL, 3 eq) in dioxane (1.0 mL) was added 1,3-bis[2,6-bis(1-ethylpropyl)phenyl]-2H-imidazole; 3-chloropyridine; dichloropalladium (8.19 mg, 10.32 μmol, 0.05 eq) and Cs₂CO₃ (201.79 mg, 619.34 μmol, 3 eq). The mixture was stirred at 100° C. for 24 hr. LCMS showed one major peak with desired mass was detected, no reactant 1 remained. The mixture was filtered to give a residue. The crude product was purified by reversed-phase HPLC (0.1% FA condition, 40 g C18 Column, Eluent of 30˜60% ACN/H₂O ether gradient @ 40 mL/min). The eluent was lyophilized to give Intermediate 18-3 (60 mg, 98.31 μmol, 47.62% yield, 94.66% purity) was obtained as a white solid, it was confirmed by LCMS. Mass Found: LCMS Retention time: 0.857 min (M+H)⁺=578.2, and Retention time: 0.813 mi (M+H)⁺=578.3.

Step 2: To a mixture of Intermediate 18-3 (60 mg, 103.86 μmol, 1 eq) in DCM (0.5 mL) and TFA (0.1 mL) was stirred at 25° C. for 2 hr. LCMS showed Reactant 1 was consumed completely and one main peak with desired mass was detected. The mixture was filtered and concentrated under reduced pressure to give a residue. The crude product was purified by reversed-phase HPLC (0.1% FA condition). The eluent was lyophilized to give Intermediate 18-4 (40 mg, 80.61 μmol, 77.62% yield, 96.25% purity) was obtained as an off-white solid, which was confirmed by LCMS. Mass Found: Retention time: 0.650 min (M+H)⁺=478.4, and Retention time: 0.652 min (M+H)⁺=478.3.

Step 3: To a solution of Intermediate 18-4 (40 mg, 67.61 μmol, 1.0 eq, TFA) and Intermediate 18-5 (48.30 mg, 202.83 μmol, 3 eq) in DMF (0.4 mL) was added K₂CO₃ (46.72 mg, 338.05 μmol, 5 eq). The mixture was stirred at 80° C. for 4 hr. LCMS Reactant 1 was consumed completely and one main peak with desired mass was detected. The mixture was filtered to give a residue. The residue was purified by prep-HPLC (column: Phenomenex luna C18 250*50 mm*15 μm; mobile phase: [water (0.2% FA)-ACN]; B %: 14%-44%, 10 min). The eluent was lyophilized to give product, HNMR showed an impurity, the crude product was purified by prep-HPLC (column: Waters Xbridge 150*25 mm*5 um; mobile phase: [water (0.05% ammonia hydroxide v/v)-ACN]; B %: 33%-63%, 9 min), The eluent was lyophilized to afford Intermediate 18-6 (30 mg, 47.26 μmol, 69.90% yield, 100% purity) was obtained as a white solid, which was confirmed by LCMS, SFC, and HNMR. Mass Found: LCMS Retention time: 0.728 min, (M+H)⁺=635.3, and Retention time: 0.724 min, (M+H)⁺=635.5; SFC data, SFC: Retention time: 1.515 min; NMR Data, 1H NMR (400 MHz, Methanol-d₄) δ=8.20-8.04 (m, 1H), 7.95-7.91 (m, 1H), 7.38-7.21 (m, 4H), 7.21-7.13 (m, 1H), 6.89 (br d, J=8.0 Hz, 1H), 6.65 (s, 1H), 4.27-4.09 (m, 1H), 4.09-3.79 (m, 2H), 3.72-3.59 (m, 1H), 3.37-3.33 (m, 2H), 3.29-3.15 (m, 2H), 3.14-3.06 (m, 2H), 3.05-2.87 (m, 1H), 2.83-2.71 (m, 1H), 2.68-2.65 (m, 2H), 2.63-2.44 (m, 3H), 2.31 (s, 3H), 1.74-1.46 (m, 4H), 1.42 (s, 10H), 1.36-1.28 (m, 4H).

### Example 19. Synthesis of (R)-tert-butyl (3-((2-((3-((4-hydroxy-1-(3-phenylbutanoyl)piperidin-4-yl)methyl)-4-oxo-3,4-dihydroquinazolin-7-yl)(methyl)amino)ethyl)(methyl)amino)propyl)carbamate (Intermediate 19-5)

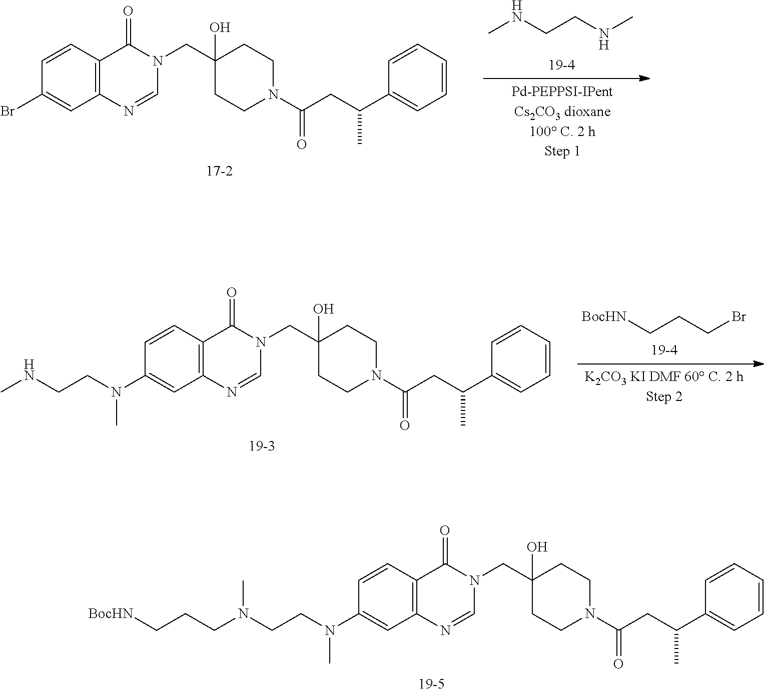

Step 1: To a solution of Intermediate 17-2 (100 mg, 206.45 μmol, 1 eq) and Intermediate 19-2 (90.99 mg, 1.03 mmol, 111.10 μL, 5 eq) in dioxane (1 mL) was added 1,3-bis[2,6-bis(1-ethylpropyl)phenyl]-2H-imidazole; 3-chloropyridine;dichloropalladium (8.19 mg, 10.32 μmol, 0.05 eq) and Cs₂CO₃ (201.79 mg, 619.35 μmol, 3 eq). The mixture was stirred at 100° C. for 2 hr. LCMS showed one major peak with desired mass was detected, no reactant 1 remained. The mixture was filtered to give a residue. The crude product was purified by reversed-phase HPLC (0.1% FA condition, 40 g C18 Column, Eluent of 30˜60% ACN/H₂O ether gradient @ 40 mL/min). The eluent was lyophilized to give Intermediate 19-3 (80 mg, 148.89 μmol, 72.12% yield, 91.5% purity) was obtained as a white solid, it was confirmed by LCMS and, HNMR. Mass Found: LCMS Retention time: 0.679 min, (M+H)⁺=492.3, and Retention time: 0.671 min, (M+H)⁺=492.3; NMR Data, 1H NMR (400 MHz, DMSO-d₆) δ=8.08 (d, J=10.4 Hz, 1H), 7.90 (d, J=9.2 Hz, 1H), 7.31-7.20 (m, 4H), 7.18-7.10 (m, 1H), 6.98-6.95 (m, 1H), 6.71 (d, J=2.0 Hz, 1H), 5.34-4.47 (m, 1H), 4.07-3.89 (m, 2H), 3.85 (br s, 1H), 3.58 (br s, 4H), 3.24-3.12 (m, 2H), 3.04 (s, 3H), 2.94-2.82 (m, 1H), 2.78 (br s, 2H), 2.66-2.52 (m, 2H), 2.38 (br s, 3H), 1.55-1.24 (m, 4H), 1.19 (br d, J=6.8 Hz, 3H).

Step 2: To a solution of Intermediate 19-3 (77.50 mg, 325.45 μmol, 2.0 eq) in DMF (1 mL) was added K₂CO₃ (67.47 mg, 488.18 μmol, 3 eq) and KI (5.40 mg, 32.55 μmol, 0.2 eq). The mixture was stirred at 60° C. for 2 hr. LCMS showed Reactant 1 was consumed completely and one main peak with desired m/z or desired mass was detected. The mixture was filtered to give a residue. The residue was purified by prep-HPLC (column: Phenomenex luna C18 250*50 mm*15 μm; mobile phase: [water (0.2% FA)-ACN]; B %: 18%-38%, 10 min). The eluent was lyophilized to afford Intermediate 19-5 (80 mg, 111.32 μmol, 68.41% yield, 96.69% purity, FA) was obtained as an off-white solid, which was confirmed by LCMS, HNMR, and SFC. Mass Found: LCMS Retention time: 0.736 min, (M+H)⁺=649.3, and Retention time: 0.734 min, (M+H)⁺=649.3; SFC data, SFC: Retention time: 2.613 min; NMR Data, 1H NMR (400 MHz, DMSO+D₂O) δ=8.05 (br d, J=14.4 Hz, 1H), 7.92 (br d, J=8.8 Hz, 1H), 7.27-7.16 (m, 4H), 7.16-7.07 (m, 1H), 6.97 (br d, J=8.8 Hz, 1H), 6.68 (s, 1H), 3.84-3.71 (m, 3H), 3.66-3.51 (m, 3H), 3.44-3.32 (m, 1H), 3.24-3.05 (m, 2H), 3.01 (s, 3H), 2.96-2.76 (m, 4H), 2.65 (br s, 2H), 2.62-2.54 (m, 2H), 2.38 (br s, 3H), 1.63-1.42 (m, 3H), 1.35-1.30 (m, 12H), 1.19-1.16 (m, 3H).

### Example 20. Synthesis of 7-chloro-3-((4-hydroxypiperidin-4-yl)methyl)quinazolin-4(3H)-one (Intermediate 20-5)

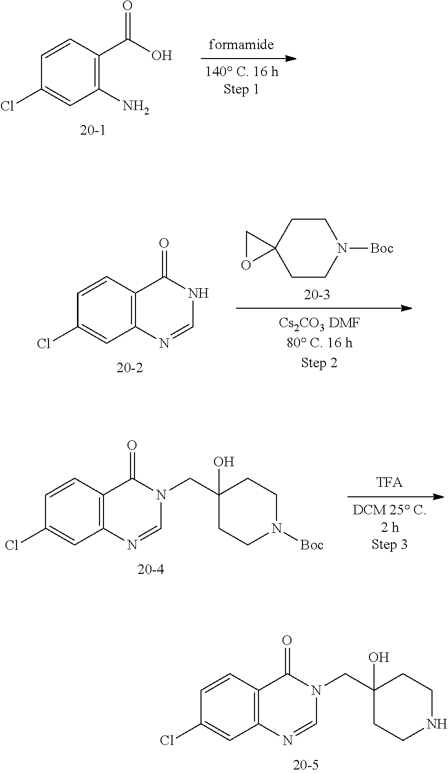 [SMILES: Nc1cc(Cl)ccc1C(=O)O ; O=c1[nH]cnc2cc(Cl)ccc12 ; CC(C)(C)OC(=O)N1CCC2(CC1)CO2 ; CC(C)(C)OC(=O)N1CCC(O)(Cn2cnc3cc(Cl)ccc3c2=O)CC1 ; O=c1c2ccc(Cl)cc2ncn1CC1(O)CCNCC1]

Step 1: To a mixture of Intermediate 20-1 (3.0 g, 17.48 mmol, 1 eq) in formamide (4 mL) was stirred at 140° C. for 16 hr. LCMS showed Intermediate 20-1 was consumed completely and one major peak with desired mass was detected. The reaction mixture was diluted with H₂O (10 mL) and the mixture was filtered and concentrated to give a brown solid, which was the crude product Intermediate 20-2 (2.4 g, 12.97 mmol, 74.21% yield, 97.63% purity) as a brown solid, it was used into the next step without further purification. It was confirmed by LCMS. Mass Found, LCMS: Retention time: 0.619 min, (M+H)=180.9, and LCMS: Retention time: 0.616 min, (M+H)=180.9; NMR Data, 1H NMR (400 MHz, DMSO-d₆) δ=12.38 (br s, 1H), 8.22-7.94 (m, 2H), 7.71 (d, J=2.0 Hz, 1H), 7.55-7.52 (m, 1H).

Step 2: To a solution of Intermediate 20-2 (1.9 g, 10.52 mmol, 1 eq) and tert-butyl 1-oxa-6-azaspiro[2.5]octane-6-carboxylate (2.69 g, 12.63 mmol, 1.2 eq) in DMF (19 mL) was added Cs₂CO₃ (10.28 g, 31.56 mmol, 3 eq). The mixture was stirred at 80° C. for 16 hr. LCMS showed Intermediate 20-2 was consumed completely and one major peak with desired mass was detected. The reaction mixture was diluted with H₂O (10 mL) and extracted with EA 60 mL (20 mL*3), combined all organic layers were washed with brine (50 mL). Then dried over Na₂SO₄, filtered and concentrated under reduced pressure to give a residue. The residue was purified by flash silica gel chromatography (ISCO®; 80 g SepaFlash® Silica Flash Column, Eluent of 60˜90% Ethyl acetate/Petroleum ether gradient @ 80 mL/min). Intermediate 20-4 (3.0 g, 7.62 mmol, 72.40% yield) was obtained as a white solid, which was confirmed by LCMS and HNMR. Mass Found, LCMS: Retention time: 0.852 min, (M+H−56)=337.9, and LCMS: Retention time: 0.853 min, (M+H−56)=337.9; NMR Data, 1H NMR (400 MHz, DMSO-d6) δ=8.28 (s, 1H), 8.15 (d, J=8.6 Hz, 1H), 7.75 (d, J=2.0 Hz, 1H), 7.58 (dd, J=2.1, 8.6 Hz, 1H), 4.92 (s, 1H), 4.06-3.94 (m, 2H), 3.65 (br d, J=12.8 Hz, 2H), 3.05 (br d, J=6.4 Hz, 2H), 1.53-1.43 (m, 2H), 1.39 (s, 11H).

Step 3: To a mixture of Intermediate 20-4 (3.0 g, 7.62 mmol, 1 eq) in TFA (5 mL) and DCM (25 mL) was stirred at 25° C. for 2 hr. LCMS showed Intermediate 20-4 was consumed completely and one major peak with desired mass was detected. The reaction mixture was concentrated under reduced pressure to give a residue. The residue was purified by prep-HPLC (column: Kromasil Eternity XT 250*80 mm*10 μm; mobile phase: [water (10 mM NH₄HCO₃)-ACN]; B %: 18ACN %-48%, 10 min). Intermediate 20-5 (2.7 g, 6.45 mmol, 84.63% yield, 97.35% purity) was obtained as a white solid, which was confirmed by LCMS, HNMR, and FNMR. Mass Found, LCMS: Retention time: 0.525 min, (M+H)=294.0, and LCMS: Retention time: 0.538 min, (M+H)=294.0; NMR Data, 1H NMR (400 MHz, Methanol-d₄) δ=8.29 (s, 1H), 8.22 (d, J=8.8 Hz, 1H), 7.70 (d, J=2.0 Hz, 1H), 7.60-7.53 (m, 1H), 4.14 (s, 2H), 3.22-3.11 (m, 4H), 1.97-1.81 (m, 2H), 1.70 (br d, J=13.2 Hz, 2H); 19F NMR (377 MHz, Methanol-d4).

### Example 21. Synthesis of (R)-7-chloro-3-((4-hydroxy-1-(3-phenylbutanoyl)piperidin-4-yl)methyl)quinazolin-4(3H)-one (Intermediate 21-3)

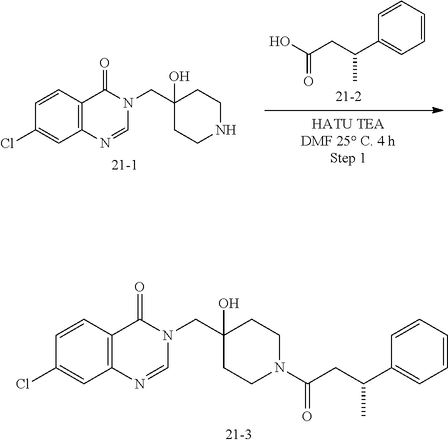 [SMILES: O=c1c2ccc(Cl)cc2ncn1CC1(O)CCNCC1 ; C[C@H](CC(=O)O)c1ccccc1 ; C[C@H](CC(=O)N1CCC(O)(Cn2cnc3cc(Cl)ccc3c2=O)CC1)c1ccccc1]

Step 1: To a solution of Intermediate 21-1 (200 mg, 490.47 μmol, 1 eq, TFA) and 3-phenylbutanoic acid (96.64 mg, 588.57 μmol, 63.58 μL, 1.2 eq) in DMF (2 mL) was added HATU (372.98 mg, 980.94 μmol, 2 eq) and TEA (148.89 mg, 1.47 mmol, 204.80 μL, 3 eq). The mixture was stirred at 25° C. for 4 hr. LCMS showed no Intermediate 21-1 remained and 49.66% of desired mass was detected. The reaction mixture was diluted with H₂O (5 mL) and extracted with EA 30 mL (10 mL*3). Then diluted with saturation of NaCl (5 mL) and dried over Na₂SO₄, filtered and concentrated under reduced pressure to give a residue. The residue was purified by prep-HPLC (column: Phenomenex luna C18 250*50 mm*15 um; mobile phase: [water (0.2% FA)-ACN]; B %: 38%-68%, 10 min). Intermediate 21-3 (120 mg, 261.60 μmol, 53.34% yield, 95.906% purity) was obtained as a yellow solid, it was confirmed by LCMS, SFC, and HNMR. Mass Found: LCMS Retention time: 0.851 min, (M+H)⁺=439.9, and Retention time: 0.852 min, (M+H)⁺=440.0; SFC data, SFC: Retention time: 1.438 min; NMR Data, 1H NMR (400 MHz, DMSO-d₆) δ=8.27 (d, J=10.4 Hz, 1H), 8.16 (d, J=8.8 Hz, 1H), 7.75 (d, J=2.0 Hz, 1H), 7.59-7.57 (m, 1H), 7.29-7.22 (m, 4H), 7.17-7.13 (m, 1H), 4.93 (br d, J=4.0 Hz, 1H), 4.09-3.95 (m, 2H), 3.94-3.88 (m, 1H), 3.72-3.59 (m, 1H), 3.22-3.11 (m, 2H), 2.91-2.80 (m, 1H), 2.64-2.53 (m, 2H), 1.58-1.26 (m, 4H), 1.20 (d, J=6.0 Hz, 3H).

### Example 22. Synthesis of tert-butyl (2-(1,3-dioxo-3,4-dihydroisoquinolin-2(1H)-yl)ethyl)carbamate (Intermediate 22-3)

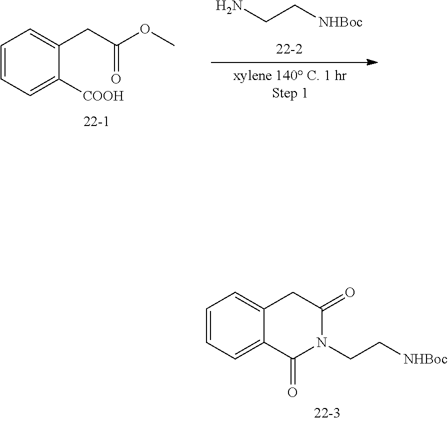 [SMILES: CC(C)(C)OC(=O)NCCN ; COC(=O)Cc1ccccc1C(=O)O ; CC(C)(C)OC(=O)NCCN1C(=O)Cc2ccccc2C1=O]

Step 1: To a solution of Intermediate 22-1 (0.5 g, 2.57 mmol, 1 eq) and Intermediate 22-2 (412.53 mg, 2.57 mmol, 404.44 μL, 1 eq) in xylene (10 mL) was stirred at 140° C. for 1 hr. LCMS showed desired molecular weight was detected. The mixture was concentrated to give crude product. The residue was purified by flash silica gel chromatography (ISCO®; 40 g SepaFlash® Silica Flash Column, Eluent of 0-100% EA/PE, PE/EA=1:1, Rf=0.6) and the eluent was concentrated to give Intermediate 22-3 (0.55 g, 1.69 mmol, 65.49% yield, 93.311% purity) as yellow solid and confirmed by LCMS and HNMR. Mass Found, LCMS: Retention time: 0.802 min, (M+H−100)=205.1, and LCMS: Retention time: 0.871 min, (M+H−100)=205.0; NMR Data, ¹H NMR (400 MHz, DMSO-d₆) δ=8.05 (d, J=8.0 Hz, 1H), 7.71-7.59 (m, 1H), 7.52-7.44 (m, 1H), 7.39 (d, J=7.6 Hz, 1H), 6.86-6.82 (m, 1H), 4.09 (s, 2H), 3.96-3.92 (m, 2H), 3.18-3.12 (m, 2H), 1.29 (s, 9H).

### Example 23. Synthesis of N-(2-aminoethyl)-3-(1,3-dioxo-3,4-dihydroisoquinolin-2(1H)-yl)propanamide (Intermediate 23-2)

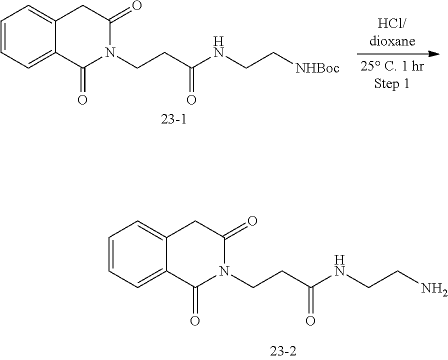 [SMILES: CC(C)(C)OC(=O)NCCNC(=O)CCN1C(=O)Cc2ccccc2C1=O ; NCCNC(=O)CCN1C(=O)Cc2ccccc2C1=O]

Step 1: To a solution of Intermediate 23-1 (0.73 g, 1.94 mmol, 1 eq) in HCl/dioxane (4 M, 7.3 mL, 15.02 eq) was stirred at 25° C. for 1 hr. LCMS showed desired molecular weight was detected. The mixture was concentrated to give a yellow solid. The residue was purified by prep-HPLC (FA condition) and lyophilized to give Intermediate 23-2 (0.316 g, 993.32 μmol, 51.08% yield, 98% purity, HCl salt) as brown solid and confirmed by LCMS, HPLC, and HNMR. Mass Found, LCMS: Retention time: 0.624 min, (M+H)=276.1, and LCMS: Retention time: 0.103 min, (M+H)=276.1; NMR Data, ¹H NMR (400 MHz, DMSO+D₂O) δ=8.03 (d, J=8.0 Hz, 1H), 7.65 (t, J=7.6 Hz, 1H), 7.50-7.44 (m, 1H), 7.38 (d, J=7.6 Hz, 1H), 4.07 (t, J=7.2 Hz, 2H), 4.03-4.00 (m, 2H), 3.25 (t, J=6.0 Hz, 2H), 2.84 (t, J=6.0 Hz, 2H), 2.37 (t, J=7.2 Hz, 2H).

### Example 24. Synthesis of 4-(4-iodophenyl)butanoic acid (Intermediate 24-2)

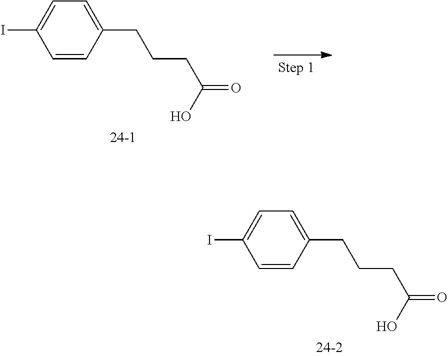 [SMILES: O=C(O)CCCc1ccc(I)cc1 ; O=C(O)CCCc1ccc(I)cc1]

LCMS Retention time: 0.441 min, (M−H)=288.9; NMR Data: ¹H NMR (400 MHz, DMSO-d₆) δ=7.83-7.82 (m, 1H), 7.63 (d, J=8.0 Hz, 2H), 7.02 (d, J=8.0 Hz, 2H), 6.75-6.73 (m, 1H), 3.52-3.45 (m, 8H), 3.41-3.35 (m, 4H), 3.19-3.18 (m, 2H), 3.06-3.03 (m, 2H), 2.52 (br s, 1H), 2.49 (br s, 1H), 2.06-2.04 (m, 2H), 1.76-1.73 (m, 2H), 1.37 (s, 9H).

### Example 25. Synthesis of 5-hexylthiophene-2-carboxylic acid (Intermediate 25-2)

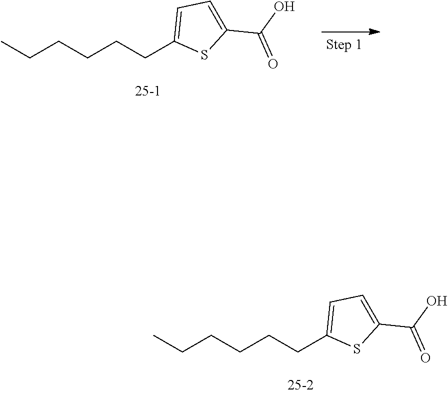

0.678 min, (M−H)=211.0; NMR data: 1H NMR (400 MHz, DMSO-d₆) δ=13.61-12.11 (m, 1H), 7.55 (d, J=3.6 Hz, 1H), 6.91 (d, J=3.6 Hz, 1H), 2.81-2.78 (m, 2H), 1.61-1.57 (m, 2H), 1.37-1.20 (m, 6H), 0.90-0.79 (m, 3H).

### Example 26. Synthesis of 4-(4-iodophenyl)-N-methylbutanamide (Intermediate 26-3)

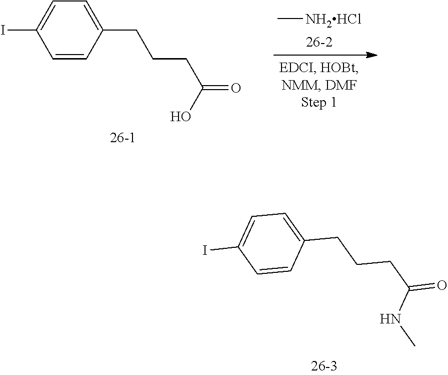

Step 1: To a solution of Intermediate 26-1 (0.2 g, 689.43 μmol, 1 eq) in DMF (2 mL) was added EDCI (264.33 mg, 1.38 mmol, 2 eq) HOAt (46.92 mg, 344.71 μmol, 48.22 μL, 0.5 eq) and NMM (348.67 mg, 3.45 mmol, 378.99 μL, 5 eq) the mixture was stirred at 25° C. for 1 h, and then Intermediate 26-2 (93.10 mg, 1.38 mmol, 2 eq, HCl) was added the mixture at 25° C. The resulting mixture was stirred at 25° C. for 4 hr. LCMS showed desired molecular weight was detected. The reaction mixture was added H₂O (5 mL) and then extracted with EA (10 mL*3), the combined organic phase was washed with brine (10 mL), dried by Na₂SO₄, filtered and concentrated to get residue. The residue was purified by prep-HPLC (column: Phenomenex Synergi C18 150*25 mm*10 um; mobile phase: [water (0.225% FA)-ACN]; B %: 29%-62%, 11 min), the eluent was concentrated to remove ACN and lyophilized to afford Intermediate 26-3 (86.74 mg, 286.14 μmol, 41.50% yield, 100% purity) was obtained as a white solid and confirmed by ¹H NMR and LCMS. Mass found: LCMS Retention time: 0.854 min, (M+H)=304.0, and Retention time: 0.853 min, (M+H)=304.1; NMR Data: ¹H NMR (400 MHz, DMSO-d₆) δ=7.69 (br s, 1H), 7.64-7.58 (m, 2H), 7.01 (d, J=8.4 Hz, 2H), 3.32 (s, 3H), 2.55-2.54 (m, 2H), 2.04-2.01 (m, 2H), 1.75-1.71 (m, 2H).

### Example 27. Synthesis of 5-hexyl-N-methyl-thiophene-2-carboxamide (Intermediate 27-1)

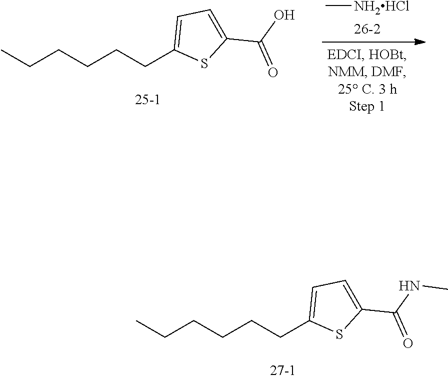

Step 1: To a solution of Intermediate 25-1 (0.1 g, 471.01 μmol, 1 eq) in DCM (1 mL) was added EDCI (180.59 mg, 942.03 μmol, 2 eq), HOAt (32.06 mg, 235.51 μmol, 32.94 μL, 0.5 eq) and NMM (238.21 mg, 2.36 mmol, 258.92 μL, 5 eq) the mixture was stirred at 25° C. for 1 h, and then Intermediate 26-2 (38.16 mg, 565.22 μmol, 1.2 eq, HCl) was added the mixture at 25° C. The resulting mixture was stirred at 25° C. for 3 hr. LCMS showed desired molecular weight was detected. The reaction mixture was concentrated under reduced pressure to give a residue. The residue was purified by prep-HPLC (column: Phenomenex Synergi C18 150*25 mm*10 um; mobile phase: [water (0.225% FA)-ACN]; B %: 42%-72%, 10 min) the eluent was concentrated to remove ACN and lyophilized to afford Intermediate 27-1 (54.33 mg, 232.75 μmol, 49.41% yield, 96.540% purity) was obtained as a white solid and confirmed by 1HNMR and LCMS. Mass found: LCMS Retention time: 0.889 min, (M+H)=226.2, and Retention time: 0.928 min, (M+H)=226.1; NMR Data: 1H NMR (400 MHz, DMSO-d₆) δ=8.29 (br d, J=4.4 Hz, 1H), 7.49 (d, J=3.6 Hz, 1H), 6.84 (d, J=3.6 Hz, 1H), 2.80-2.74 (m, 2H), 2.74-2.71 (m, 3H), 1.65-1.53 (m, 2H), 1.36-1.20 (m, 6H), 0.89-0.81 (m, 3H).

### Example 28. Synthesis of tert-butyl (16-(4-iodophenyl)-13-oxo-3, 6, 9-trioxa-12-azahexadecyl)carbamate (Intermediate 28-2)

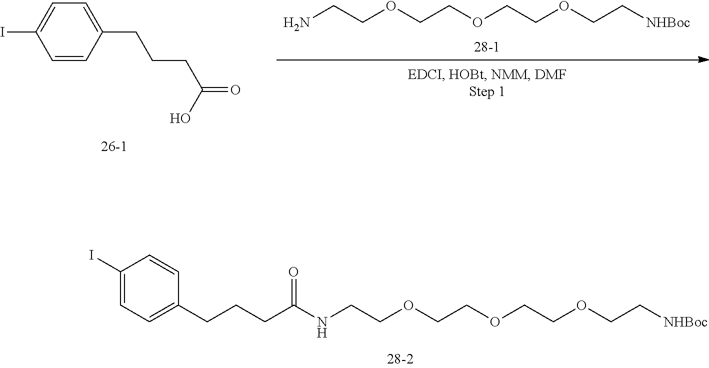 [SMILES: O=C(O)CCCc1ccc(I)cc1 ; CC(C)(C)OC(=O)NCCOCCOCCOCCN ; CC(C)(C)OC(=O)NCCOCCOCCOCCNC(=O)CCCc1ccc(I)cc1]

Step 1: To a solution of Intermediate 26-1 (0.2 g, 689.43 μmol, 1 eq) in DMF (2 mL) was added EDCI (264.33 mg, 1.38 mmol, 2 eq) HOAt (46.92 mg, 344.72 μmol, 48.22 μL, 0.5 eq) and NMM (348.67 mg, 3.45 mmol, 378.99 μL, 5 eq) the mixture was stirred at 25° C. for 1 h, and then Intermediate 28-1 (241.88 mg, 827.32 μmol, 1.2 eq) was added the mixture at 25° C. The resulting mixture was stirred at 25° C. for 2 hr. LCMS showed desired molecular weight was detected. The reaction mixture was concentrated under reduced pressure to give a residue. The residue was purified by prep-HPLC (column: Phenomenex Synergi C18 150*25 mm*10 um; mobile phase: [water (0.225% FA)-ACN]; B %: 41%-71%, 10 min) and the eluent was concentrated to remove ACN and lyophilized to afford Intermediate 28-2 (110.13 mg, 195.11 μmol, 28.30% yield) as yellow oil and confirmed by 1HNMR and LCMS. Mass found: LCMS Retention time: 0.915 min, (M+H)=565.1, and Retention time: 0.932 min, (M+H)=565.3; NMR Data: 1H NMR (400 MHz, Chloroform-d) δ=7.62-7.57 (m, 2H), 6.95 (d, J=8.4 Hz, 2H), 6.12 (br d, J=0.8 Hz, 1H), 5.06 (br s, 1H), 3.68-3.59 (m, 8H), 3.59-3.51 (m, 4H), 3.46-3.45 (m, 2H), 3.31 (d, J=3.6 Hz, 2H), 2.60-2.58 (m, 2H), 2.18-2.16 (m, 2H), 1.99-1.90 (m, 2H), 1.45 (s, 9H).

### Example 29. Synthesis of tert-butyl N-[2-[2-[2-[2-[(5-hexylthiophene-2-carbonyl) amino]ethoxy]ethoxy]ethoxy]ethyl]carbamate (Intermediate 29-1)

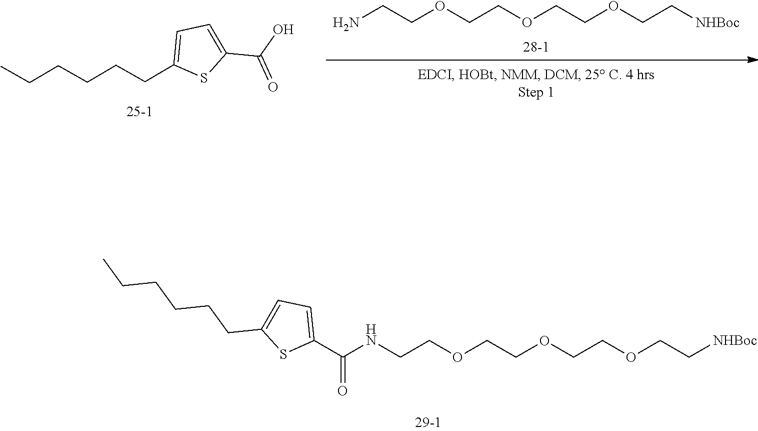 [SMILES: CC(C)(C)OC(=O)NCCOCCOCCOCCN ; CCCCCCc1ccc(C(=O)O)s1 ; CCCCCCc1ccc(C(=O)NCCOCCOCCOCCNC(=O)OC(C)(C)C)s1]

Step 1: To a solution of Intermediate 25-1 (0.1 g, 471.01 μmol, 1 eq) in DCM (0.1 mL) was added EDCI (180.59 mg, 942.03 μmol, 2 eq) HOAt (32.06 mg, 235.51 μmol, 32.94 μL, 0.5 eq) and NMM (238.21 mg, 2.36 mmol, 258.92 μL, 5 eq) the mixture was stirred at 25° C. for 1 hr, and then Intermediate 28-1 (165.25 mg, 565.22 μmol, 1.2 eq) was added the mixture at 25° C. The resulting mixture was stirred at 25° C. for 3 hrs. LCMS showed desired molecular weight was detected. The reaction mixture was concentrated under reduced pressure to give a residue. The residue was purified by prep-HPLC (column: Phenomenex Synergi C18 150*25 mm*10 μm; mobile phase: [water (0.225% FA)-ACN]; B %: 51%-81%, 11 min) and the eluent was concentrated to remove ACN and lyophilized to afford Intermediate 29-1 (108.9 mg, 223.77 μmol, 47.51% yield, 100% purity) as yellow oil which was confirmed by HNMR and LCMS. Mass Found, LCMS: Retention time: 0.964 min, (M+H)=487.3, and LCMS: Retention time: 1.008 min, (M+H)=487.4; NMR Data, 1H NMR (400 MHz, Methanol-d₄) δ=7.51 (d, J=3.6 Hz, 1H), 6.82 (d, J=3.6 Hz, 1H), 6.64-6.54 (m, 1H), 3.67-3.61 (m, 8H), 3.60-3.55 (m, 2H), 3.55-3.50 (m, 2H), 3.48-3.46 (m, 2H), 3.23-3.17 (m, 2H), 2.84-2.81 (m, 2H), 1.73-1.64 (m, 2H), 1.43 (s, 9H), 1.39 (d, J=7.2 Hz, 6H), 0.95-0.87 (m, 3H).

### Example 30. Synthesis of 3-(1,3-dioxo-1,2,3,4-tetrahydroisoquinolin-4-yl)propanoic acid (Intermediate 30-3)

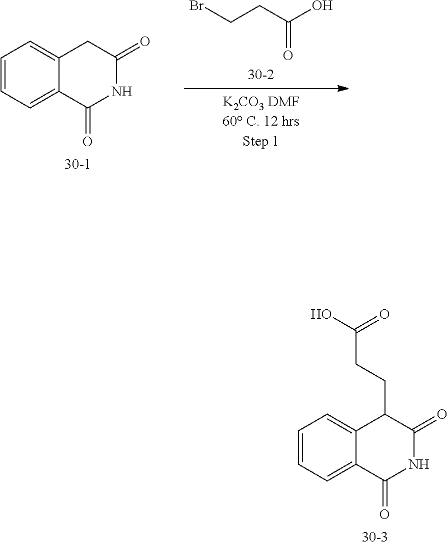 [SMILES: O=C1Cc2ccccc2C(=O)N1 ; O=C(O)CCBr ; O=C(O)CCC1C(=O)NC(=O)c2ccccc21]

Step 1: To a solution of Intermediate 30-1 (0.1 g, 620.51 μmol, 1 eq) and Intermediate 30-2 (113.91 mg, 744.62 μmol, 76.96 μL, 1.2 eq) in DMF (1 mL) was added K₂CO₃ (171.52 mg, 1.24 mmol, 2 eq), then the mixture was stirred at 60° C. for 12 hrs. LCMS showed desired molecular weight was detected. The mixture was wash with water (1 mL) and extracted with EA (1 mL*3). The combined organic layers were washed with brine 3 mL, dried over anhydrous sodium sulfate, filtered and concentrated under reduced pressure to give a residue. The residue was purified by flash silica gel chromatography (ISCO®; 12 g SepaFlash® Silica Flash Column, Eluent of 0-100% EA/MeOH, EA/MeOH=1:1, Rf=0.5) and the eluent was concentrated to afford Intermediate 30-3 (0.02 g, 85.76 μmol, 13.82% yield) was colorless oil and confirmed by HNMR, LCMS and 2D NMR. Mass Found, LCMS: Retention time: 0.591 min, (M+H)=234.3; and LCMS: Retention time: 0.750 min, (M+H−17)=216.2; NMR Data, ¹H NMR (400 MHz, DMSO-d₆) δ=7.89-7.86 (m, 1H), 7.60-7.56 (m, 1H), 7.42-7.38 (m, 1H), 7.34 (d, J=8.0 Hz, 1H), 3.88-3.85 (m, 1H), 2.87-2.74 (m, 2H), 2.27-2.20 (m, 2H).

### Example 31. Synthesis of 1-((7-(5-chloro-3-methyl-2-(piperidin-4-yloxy)phenyl)thieno[3,2-b]pyridin-2-yl)methyl)pyrrolidine-2,5-dione (Intermediate 31-2)

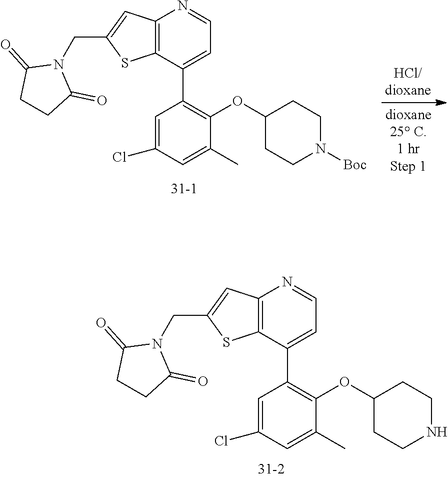 [SMILES: Cc1cc(Cl)cc(-c2ccnc3cc(CN4C(=O)CCC4=O)sc23)c1OC1CCN(C(=O)OC(C)(C)C)CC1 ; Cc1cc(Cl)cc(-c2ccnc3cc(CN4C(=O)CCC4=O)sc23)c1OC1CCNCC1]

Step 1: To a solution of Intermediate 31-1 (0.05 g, 87.70 μmol, 1 eq) in dioxane (0.2 mL) was added HCl/dioxane (4 M, 0.3 mL, 13.68 eq) at 25° C., then the mixture was stirred at 25° C. for 1 hr. LCMS showed desired mass was detected. The mixture was concentrated to give a crude product. The residue was purified by prep-HPLC (column: Phenomenex Synergi C18 150*25 mm*10 μm; mobile phase: [water (0.225% FA)-ACN]; B %: 12%-42%, 10 min) and lyophilized to give Intermediate 31-2 (0.0119 g, 23.06 μmol, 26.29% yield, 100% purity, FA) as yellow gum and confirmed by LCMS and HNMR. Mass Found, LCMS: Retention time: 0.704 min, (M+H)=470.0, and LCMS: Retention time: 0.806 min, (M+H)=469.9; NMR Data, ¹H NMR (400 MHz, CHLOROFORM-d) δ=8.61 (d, J=4.8 Hz, 1H), 8.26 (s, 1H), 7.48 (s, 1H), 7.22 (d, J=2.0 Hz, 1H), 7.20-7.15 (m, 2H), 4.88 (s, 2H), 3.53 (s, 1H), 2.71 (s, 4H), 2.65 (d, J=8.8 Hz, 2H), 2.59-2.49 (m, 2H), 2.27 (s, 3H), 1.64-1.53 (m, 2H), 1.47 (d, J=3.2 Hz, 2H).

### Example 32. Synthesis of 4-(2-amino-4-ethyl-5-(1H-indazol-5-yl)pyridin-3-yl)phenol (Intermediate 32-3)

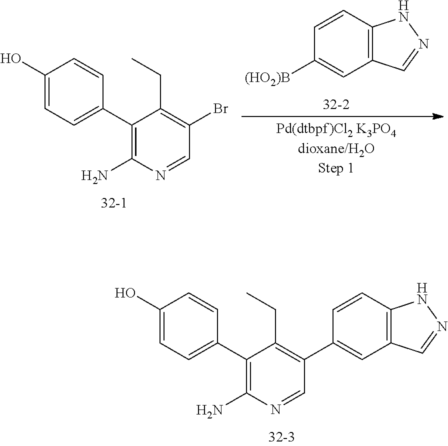

Step 1: To a solution of Intermediate 32-1 (0.05 g, 170.56 μmol, 1 eq) and Intermediate 32-2 (27.62 mg, 170.56 μmol, 1 eq) in dioxane (0.5 mL) and H₂O (0.125 mL) was added K₃PO₄ (72.41 mg, 341.11 μmol, 2 eq) and di-tert-butyl(cyclopentyl)phosphane;dichloropalladium;iron (11.12 mg, 17.06 μmol, 0.1 eq). The reaction mixture was stirred at 80 C for 12 hrs. Extra Intermediate 32-2 (27.62 mg, 170.56 μmol, 1 eq) di-tertbutyl(cyclopentyl)phosphane;dichloropalladium;iron (11.12 mg, 17.06 μmol, 0.1 eq) and K₃PO₄ (72.41 mg, 341.11 μmol, 2 eq) were added into the reaction mixture. The mixture was stirred at 80 C for another 12 hrs. LCMS showed desired mass was detected. The reaction mixture was diluted with water 10 mL and extracted with EA 15 mL (5 mL*3). The combined organic layers were dried over Na₂SO₄, filtered and concentrated under reduced pressure to give a residue. The residue was purified by prep-HPLC (FA condition; column: Phenomenex Synergi C18 150*25 mm*10 μm; mobile phase: [water (0.225% FA)-ACN];B %: 0%-30%, 10 min). Intermediate 32-3 (0.03 g, 90.80 μmol, 53.24% yield, 100% purity) was obtained as a white solid. Mass Found: LCMS Retention time: 0.667 min, (M+H)=331.1, and Retention time: 0.748 min, (M+H)=331.0; NMR Data: ¹H NMR (400 MHz, Methanol-d₄) δ=8.35 (br s, 1H), 8.09 (d, J=0.8 Hz, 1H), 7.78-7.70 (m, 2H), 7.61 (d, J=8.4 Hz, 1H), 7.39-7.35 (m, 1H), 7.22-7.07 (m, 2H), 7.01-6.92 (m, 2H), 2.45-2.38 (m, 2H), 0.72-0.67 (m, 3H).

### Example 33. Synthesis of tert-butyl 4-(4-chloro-2-(2-((2,5-dioxopyrrolidin-1-yl)methyl)thieno[3,2-b]pyridin-7-yl)-6-methylphenoxy)piperidine-1-carboxylate (Intermediate 33-1)

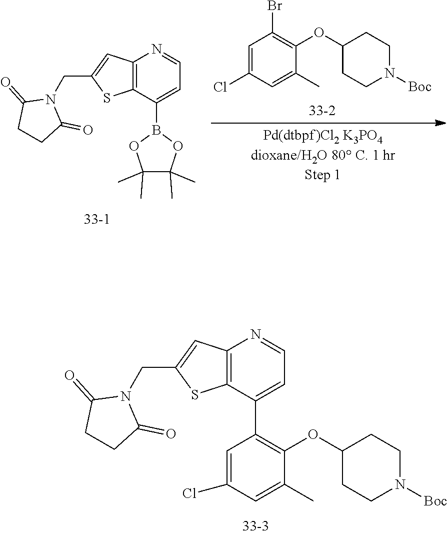 [SMILES: CC1(C)OB(c2ccnc3cc(CN4C(=O)CCC4=O)sc23)OC1(C)C ; Cc1cc(Cl)cc(Br)c1OC1CCN(C(=O)OC(C)(C)C)CC1 ; Cc1cc(Cl)cc(-c2ccnc3cc(CN4C(=O)CCC4=O)sc23)c1OC1CCN(C(=O)OC(C)(C)C)CC1]

Step 1: To a solution of Intermediate 33-1 (3.5 g, 9.40 mmol, 1 eq) and Intermediate 33-2 (3.81 g, 9.40 mmol, 1 eq) in dioxane (35 mL) and H₂O (7 mL) was added Pd(dtbpf)Cl₂ (612.80 mg, 940.24 μmol, 0.1 eq) and K₃PO₄ (5.99 g, 28.21 mmol, 3 eq), then the mixture was stirred at 80° C. for 1 hr under N₂ atmosphere. LCMS showed desired molecular weight was detected. The mixture was filtered, the organic phase was concentrated under reduced pressure to give crude product. The residue was purified by column chromatography (SiO₂, Petroleum ether/Ethyl acetate=100/0 to 0/100, PE:EA=1:1 Rt=0.2) and concentrated to give product 1 (5.3 g, 9.30 mmol, 98.88% yield) as brown oil. product 1 (30 mg, 52.62 mmol) was purified by prep-HPLC (column: Phenomenex Synergi C18 150*25 mm*10 μm; mobile phase: [water (0.225% FA)-ACN];B %: 63%-83%, 10 min) and lyophilized to give Intermediate 33-3 (0.00883 g, 15.49 μmol, 29.43% yield, 100% purity) as yellow oil and confirmed by LCMS and HNMR. Mass Found, LCMS: Retention time: 0.938 min, (M+H)=570.2, and LCMS: Retention time: 0.980 min, (M+H)=570.1; NMR Data, ¹H NMR (400 MHz, CHLOROFORM-d) δ=8.72 (d, J=4.8 Hz, 1H), 7.59 (s, 1H), 7.31-7.29 (m, 1H), 7.27 (s, 2H), 4.98 (s, 2H), 3.60-3.38 (m, 3H), 2.79 (s, 4H), 2.67-2.60 (m, 2H), 2.37 (s, 3H), 1.40 (s, 11H), 1.31-1.24 (m, 2H).

### Example 34. Synthesis of (R)-3-bromo-6-((4-hydroxy-1-(3-phenylbutanoyl)piperidin-4-yl)methyl)-2-methyl-2H-pyrazolo[4,3-d]pyrimidin-7(6H)-one (Intermediate 34-10)

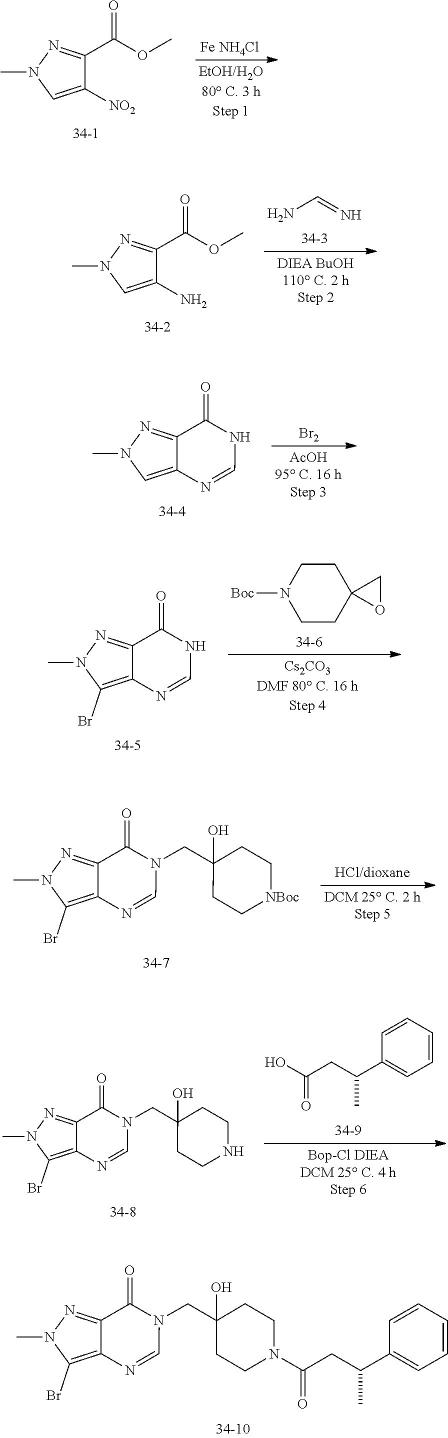

Step 1: To a solution of Intermediate 34-1 (4.5 g, 24.31 mmol, 1 eq) in EtOH (33.75 mL) and H₂O (11.25 mL) was added NH₄Cl (6.50 g, 121.53 mmol, 5 eq) and Fe (4.07 g, 72.92 mmol, 3 eq) The mixture was stirred at 80° C. for 3 hr. TLC (PE/EA=0:1) indicated Reactant 1 (R_{f}=0.55) was consumed completely and one new spot (Rf=0.30) formed. The reaction was clean according to TLC. The mixture was filtered and concentrated under reduced pressure to give a residue. Then diluted with DCM (20 mL) and dried over Na₂SO₄, filtered and concentrated under reduced pressure to give a residue as crude product Intermediate 34-2 (4.2 g, crude) was a brown solid and it used into the next step without further purification, it was confirmed by HNMR. NMR Data 1H NMR (400 MHz, DMSO-d6) δ=7.12 (s, 1H), 4.68 (br s, 2H), 3.74 (d, J=3.6 Hz, 6H).

Step 2: To a mixture of Intermediate 34-2 (4.1 g, 26.43 mmol, 1 eq) and Intermediate 34-3 (5.50 g, 52.85 mmol, 2 eq) in n-BuOH (20 mL) and DIEA (20 mL) was stirred at 110° C. for 2 hr. LCMS showed Intermediate 34-2 was consumed completely and one main peak with desired mass was detected. The reaction mixture was diluted with MTBE (50 mL), and then the mixture was filtered to give a brown solid, which was the crude product Intermediate 34-4 (4.5 g, crude) was a brown solid, and it used into the next step without further purification. It was confirmed by LCMS and HNMR. Mass Found, LCMS: Retention time: 0.184 min, (M+H)=151.0, and LCMS: Retention time: 0.183 min, (M+H)=151.0; NMR Data, 1H NMR (400 MHz, DMSO-d₆) δ=8.29 (s, 1H), 7.76 (s, 1H), 4.07 (s, 3H).

Step 3: To a solution of Intermediate 34-4 (4.3 g, 28.64 mmol, 1 eq) in AcOH (30 mL) was added Br₂ (13.73 g, 85.92 mmol, 4.43 mL, 3 eq). The mixture was stirred at 95° C. for 16 hr. LCMS showed Intermediate 34-4 was consumed completely and one major peak with desired mass was detected. The reaction mixture was diluted with H₂O (20 mL), Then MTBE (10 mL) was added in, the mixture was filtered to give a yellow solid, which was the crude product, The water phase was washed with saturated aqueous Na₂SO₃ (100 mL), until starch potassium iodide paper turn to white. The crude product Intermediate 34-5 (5.0 g, crude) was a yellow solid, and it was used into the next step without further purification. It was confirmed by HNMR. Mass Found, LCMS: Retention time: 0.595 min, (M+H)=228.9; NMR Data, 1H NMR (400 MHz, DMSO-d6) δ=12.24-11.75 (m, 1H), 7.85 (s, 1H), 4.07 (s, 3H).

Step 4: To a solution of Intermediate 34-5 (5 g, 21.83 mmol, 1 eq) and Intermediate 34-6 (5.59 g, 26.20 mmol, 1.2 eq) in DMF (50 mL) was added Cs₂CO₃ (7.11 g, 21.83 mmol, 1 eq). The mixture was stirred at 80° C. for 16 hr. LCMS showed Reactant 1 was consumed completely and one major peak with desired mass was detected. The reaction mixture was diluted with H₂O (50 mL) and extracted with EA 300 mL (100 mL*3). Then dried over Na₂SO₄, filtered and concentrated under reduced pressure to give a residue. The residue was purified by flash silica gel chromatography (ISCO®; 220 g SepaFlash® Silica Flash Column, Eluent of 50˜100% Ethyl acetate/Petroleum ether gradient @ 100 mL/min). The eluent was concentrated to give product Intermediate 34-7 (8 g, 16.10 mmol, 73.74% yield, 89% purity) was obtained as a white solid which was confirmed by LCMS, HNMR. Mass Found, LCMS: Retention time: 0.744 min, (M+H)=386.0, and LCMS: Retention time: 0.742 min, (M+H)=386.0; NMR Data, 1H NMR (400 MHz, DMSO-d₆) δ=8.04 (s, 1H), 4.86 (s, 1H), 4.08 (s, 3H), 3.96 (s, 2H), 3.65 (br d, J=12.8 Hz, 2H), 2.50-2.49 (m, 2H), 1.47-1.33 (m, 13H).

Step 5: To a mixture of Intermediate 34-7 (1.4 g, 3.17 mmol, 1 eq) in DCM (10 mL) and HCl/dioxane (4 mL, 4M) was stirred at 25° C. for 2 hr. LCMS showed Intermediate 34-7 was consumed completely and one major peak with desired mass was detected. The reaction mixture was concentrated to give a white solid, which was the crude product Intermediate 34-8 (1.0 g, crude, HCl) was a white solid, and it was used into the next step without further purification. It was confirmed by HNMR. Mass Found, LCMS: Retention time: 0.512 min, (M+H)=344.0; NMR Data, 1H NMR (400 MHz, DMSO-d₆) δ=8.13 (s, 1H), 4.08 (s, 3H), 4.03 (s, 2H), 3.18-3.04 (m, 2H), 3.03-2.91 (m, 2H), 1.85-1.71 (m, 2H), 1.56 (br d, J=14.0 Hz, 2H).

Step 6: To a solution of Intermediate 34-8 (1 g, 2.64 mmol, 1 eq, HCl) and Intermediate 34-9 (520.37 mg, 3.17 mmol, 342.35 μL, 1.2 eq) in DCM (10 mL) was added DIEA (1.71 g, 13.20 mmol, 2.30 mL, 5 eq) and BOP—Cl (806.75 mg, 3.17 mmol, 1.2 eq). The mixture was stirred at 25° C. for 4 hr. LCMS showed Intermediate 34-8 was consumed completely and one main peak with desired mass was detected. The mixture was filtered and concentrated under reduced pressure to give a residue. The crude product was purified by reversed-phase HPLC (0.1% FA condition). The eluent was lyophilized to give product. The crude product (900 mg, 1.84 mmol, 69.78% yield) was obtained as a white solid. The crude product (50 mg) was purified by prep-HPLC (column: Phenomenex C18 75*30 mm*3 μm; mobile phase: [water (FA)-ACN]; B %: 25%-55%, 7 min). The eluent was lyophilized to afford Intermediate 34-10 (40 mg, 74.05 μmol, 72.33% yield, 90.415% purity) was obtained as a white solid. It was confirmed by LCMS, HNMR, and SFC. Mass Found, LCMS: Retention time: 0.750 min, (M+H)=489.8, and LCMS: Retention time: 0.826 min, (M+H+2)=490.1; NMR Data, 1H NMR (400 MHz, DMSO-d₆) δ=8.02 (d, J=10.0 Hz, 1H), 7.30-7.22 (m, 4H), 7.17-7.14 (m, 1H), 4.87 (d, J=4.8 Hz, 1H), 4.08 (s, 3H), 4.06-3.93 (m, 2H), 3.93-3.84 (m, 1H), 3.69-3.58 (m, 1H), 3.27-3.11 (m, 2H), 2.91-2.79 (m, 1H), 2.65-2.53 (m, 2H), 1.55-1.22 (m, 4H), 1.20 (d, J=7.0 Hz, 3H).

### Example 35. Synthesis of (R)-tert-butyl 4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzylcarbamate (Intermediate 35-2)

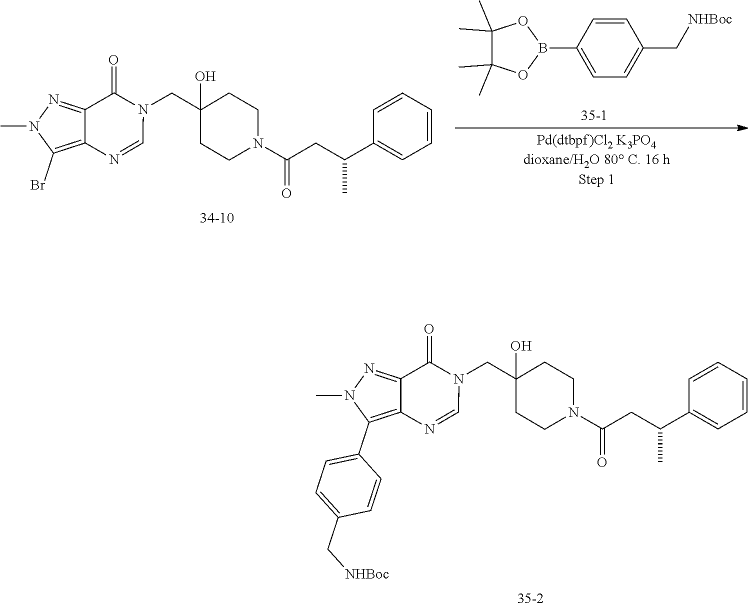 [SMILES: C[C@H](CC(=O)N1CCC(O)(Cn2cnc3c(Br)n(C)nc3c2=O)CC1)c1ccccc1 ; CC(C)(C)OC(=O)NCc1ccc(B2OC(C)(C)C(C)(C)O2)cc1 ; C[C@H](CC(=O)N1CCC(O)(Cn2cnc3c(-c4ccc(CNC(=O)OC(C)(C)C)cc4)n(C)nc3c2=O)CC1)c1ccccc1]

Step 1: To a solution of Intermediate 34-10 (50 mg, 102.38 μmol, 1 eq) and Intermediate 35-1 (68.23 mg, 204.76 μmol, 2 eq) in dioxane (0.4 mL) and H₂O (0.1 mL) was added di-tert-butyl(cyclopentyl)phosphane;dichloropalladium;iron (6.67 mg, 10.24 μmol, 0.1 eq) and K₃PO₄ (65.20 mg, 307.14 μmol, 3 eq). The mixture was stirred at 80° C. for 16 hr. LCMS showed Reactant 1 was consumed completely and one major peak with desired mass was detected. The reaction mixture was diluted with H₂O (5 mL) and extracted with EA 30 mL (10 mL*3). Then dried over Na₂SO₄ and filtered and concentrated under reduced pressure to give a residue. The residue was purified by prep-HPLC (column: Phenomenex Synergi C18 150*25 mm*10 μm; mobile phase: [water (0.225% FA)-ACN]; B %: 42%-72%, 10 min). The eluent was lyophilized to give Intermediate 35-2 (30 mg, 46.37 μmol, 45.29% yield, 95.02% purity) was obtained as a yellow solid. It was confirmed by HNMR, SFC and LCMS. Mass Found: LCMS: Retention time: 0.859 min, (M+H)⁺=615.3, and LCMS: Retention time: 0.859 min, (M+H)⁺=615.3; SFC data, SFC: Retention time: 2.312 min; NMR Data, 1H NMR (400 MHz, DMSO-d₆) δ=7.98 (d, J=10.2 Hz, 1H), 7.67 (br d, J=8.0 Hz, 2H), 7.52-7.49 (m, 1H), 7.44 (br d, J=8.0 Hz, 2H), 7.31-7.23 (m, 4H), 7.21-7.12 (m, 1H), 4.89 (br s, 1H), 4.23 (br d, J=6.0 Hz, 2H), 4.18-4.07 (m, 3H), 4.07-3.87 (m, 3H), 3.71-3.59 (m, 1H), 3.22-3.10 (m, 2H), 2.95-2.81 (m, 1H), 2.67-2.54 (m, 2H), 1.56-1.25 (m, 13H), 1.21 (d, J=6.8 Hz, 3H).

### Example 36. Synthesis of (R)-1-(3,6-dibromo-9H-carbazol-9-yl)-3-(piperazin-1-yl)propan-2-ol bis 2,2,2 trifluoroacetic acid (Intermediate 36)

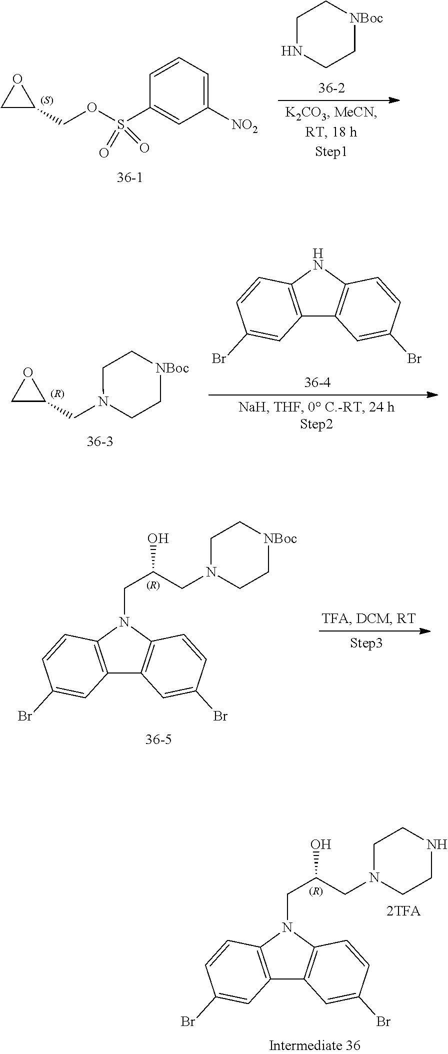

### Step 1: tert-butyl (R)-4-(oxiran-2-ylmethyl)piperazine-1-carboxylate (36-3)

To a stirred solution of (S)-oxiran-2-ylmethyl 3-nitrobenzenesulfonate (36-1, 10.72 g, 40.9 mmol) in acetonitrile (140 mL) at RT, potassium carbonate (5.71 g, 40.9 mmol) and tert-butyl piperazine-1-carboxylate (36-2, 7.0 g, 37.2 mmol) were added, and the reaction mixture was stirred at RT for 16 h. After completion (monitored by TLC), the reaction mixture was filtered and the solid was washed with acetonitrile. The combined filtrated was concentrated under reduced pressure. The crude residue was purified by flash column chromatography using 230-400 mesh silica gel eluting with 0-10% MeOH in DCM as gradient to afford the title compound (36-3, 4.5 g, 39% yield) as a pale-yellow oil. ¹H NMR (300 MHz, DMSO-d₆) δ=3.32 (t, J=5.1 Hz, 4H), 3.09-2.95 (m, 1H), 2.74-2.60 (m, 2H), 2.46-2.31 (m, 5H), 2.26-2.15 (m, 1H), 1.39 (s, 9H). LCMS: (Method C) 243.2 (M+H)⁺, Rt. 2.17 min, 77.45% (Max).

### Step 2: tert-butyl (R)-4-(3-(3,6-dibromo-9H-carbazol-9-yl)-2-hydroxypropyl)piperazine-1-carboxylate (36-5)

To a stirred solution of 3,6-dibromo-9H-carbazole (36-4, 3 g, 9.23 mmol) in THE (60 mL) at 0° C., sodium hydride (0.554 g, 13.85 mmol, 60% suspension) was added in portions over 5 min. After 5 minutes of stirring, tert-butyl (R)-4-(oxiran-2-ylmethyl)piperazine-1-carboxylate (36-3, 2.24 g, 9.23 mmol) was added, the temperature was slowly increased to RT and stirring was continued for 16 h at RT. After completion of reaction (monitored by TLC), the reaction mixture was quenched with ice-cold water (30 mL) and extracted with EtOAc (2×40 mL). The combined organic extract was washed with brine (30 mL), dried over anhydrous sodium sulphate, filtered, and concentrated under reduced pressure. The crude residue was purified by flash column chromatography (silica-gel, 230-400 mesh) using EtOAc in Pet ether (50 to 70%) as an eluent to afford the title compound (36-5, 2.8 g, 53% Yield) as an off-white solid. ¹H NMR (300 MHz, DMSO-d₆) δ=8.45 (d, J=1.1 Hz, 2H), 7.67-7.56 (m, 4H), 4.99 (d, J=5.0 Hz, 1H), 4.51-4.40 (m, 1H), 4.30 (dd, J=6.7, 14.8 Hz, 1H), 4.08-4.00 (m, 1H), 3.32-3.25 (m, 4H), 2.44-2.23 (m, 6H), 1.39 (s, 9H). LCMS: (Method B) 568.0 (M+H)⁺, Rt. 2.15 min, 99.61% (Max).

### Step 3: (R)-1-(3,6-dibromo-9H-carbazol-9-yl)-3-(piperazin-1-yl)propan-2-ol bis(2,2,2-trifluoroacetate) (Intermediate 36)

To a stirred solution of tert-butyl (R)-4-(3-(3,6-dibromo-9H-carbazol-9-yl)-2-hydroxypropyl)piperazine-1-carboxylate (36-5, 4.0 g, 7.05 mmol) in DCM (68.2 mL) at 0° C., was added trifluoroacetic acid (9.26 mL, 120 mmol) slowly and the reaction mixture was stirred for 3 h at RT. The reaction was monitored by LCMS and the starting material was consumed. The reaction mixture was concentrated, the residue was suspended in MTBE and the mixture was stirred for 30 min at RT. The solvent was decanted, and the residue was dried under vacuum to afford the title compound (6, 3.8 g, 73% yield) as an off-white solid. ¹H NMR (300 MHz, DMSO-d₆) δ=9.06-8.78 (m, 2H), 8.49 (d, J=1.6 Hz, 2H), 7.70-7.59 (m, 4H), 4.50-4.17 (m, 5H), 3.34 (m, 5H), 3.07-2.91 (m, 3H). LCMS: (Method C) 467.8 (M+H)⁺, Rt. 3.06 min, 94.54% (Max).

### Example 37. Synthesis of (R)-1-(3,6-dibromo-9H-carbazol-9-yl)-3-(piperazin-1-yl)propan-2-ol dihydrochloride (Intermediate 37)

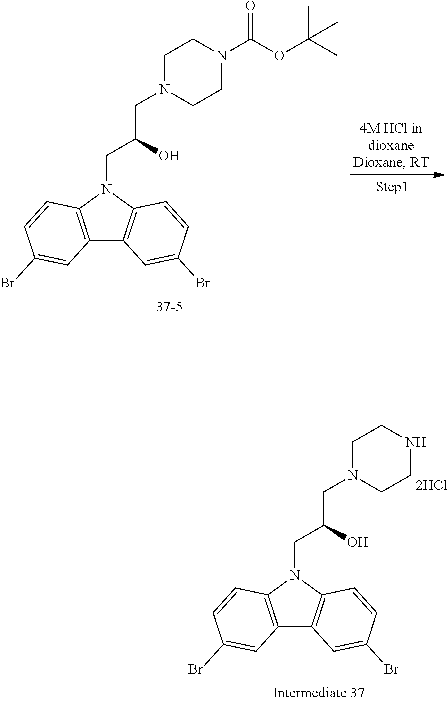 [SMILES: CC(C)(C)OC(=O)N1CCN(C[C@@H](O)Cn2c3ccc(Br)cc3c3cc(Br)ccc32)CC1 ; O[C@H](CN1CCNCC1)Cn1c2ccc(Br)cc2c2cc(Br)ccc21]

A solution of tert-butyl (R)-4-(3-(3,6-dibromo-9H-carbazol-9-yl)-2-hydroxypropyl)piperazine-1-carboxylate (37-5, 2 g, 3.53 mmol) in dioxane (40 mL) was cooled to 0° C. To the solution was added HCl in dioxane (17.63 mL, 4 M, 70.5 mmol) and the resulting mixture was stirred for 3 h at RT. After completion (monitored by TLC), the reaction mixture concentrated, and the residue was suspended in MTBE, and the mixture was stirred for 30 min at RT. The mixture was filtered to collect the solid which was dried under vacuum to get the title compound (1.92 g, 86% yield) as an off-white solid. ¹H NMR (300 MHz, DMSO-d₆) δ=11.27-10.56 (m, 0.5H), 9.99-9.12 (m, 2H), 8.49 (d, J=1.9 Hz, 2H), 7.75-7.68 (m, 2H), 7.65-7.58 (m, 2H), 6.12-5.55 (m, 0.5H), 4.42 (br s, 3H), 3.51-3.45 (m, 4H), 3.41-3.38 (m, 4H). LCMS: (Method A) 467.6 (M+H)⁺, Rt. 1.68 min, 97.92% (Max).

### Example 38. Synthesis of [[5-methyl-N-(6-(thiophene-2-sulfonamido)benzo[d]thiazol-2-yl)-5,6,7,8-tetrahydro-4H-pyrazolo[1,5-a][1,4]diazepine-2-carboxamide]] (Intermediate 38)

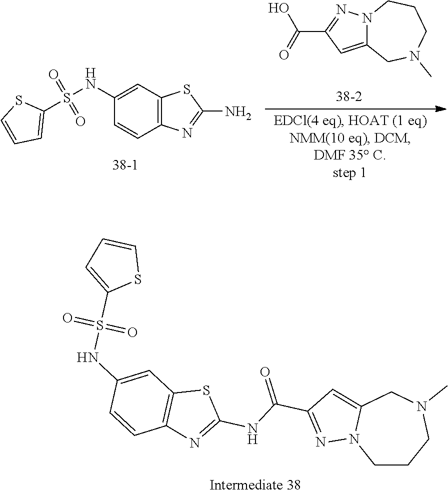 [SMILES: Nc1nc2ccc(NS(=O)(=O)c3cccs3)cc2s1 ; CN1CCCn2nc(C(=O)O)cc2C1 ; CN1CCCn2nc(C(=O)Nc3nc4ccc(NS(=O)(=O)c5cccs5)cc4s3)cc2C1]

### Step 1: Synthesis of Intermediate 38

Detailed Synthetic Procedure: To a solution of 38-1 (75.2 mg, 385 umol, 1.50 eq.) in DCM (1.50 mL) was added EDCI (197 mg, 1.03 mmol, 4.00 eq.), HOAt (35.0 mg, 257 umol, 35.9 uL, 1.00 eq.) and NMM (260 mg, 2.57 mmol, 282 μL, 10.0 eq.) and then the mixture was stirred at 25° C. for 30 min. Then 38-2 (80.0 mg, 257.0 umol, 1.00 eq.) which dissolved in DMF (0.50 mL) was added into the previous mixture and the solution was stirred at 35° C. for 2.5 hr. LCMS showed desired molecular weight was detected. The reaction mixture was filtered and filtrate was concentrated to give crude product. The crude product was dissolved in MeOH (1 ml) and purified by prep-HPLC directly (column: Phenomenex luna C18 150×25 mm×10 um; mobile phase: [water (HCl)-ACN]; B %: 9%-39%, 10 min) to give compound 1 (22.3 mg, 43.7 umol, 17.0% yield, 95.7% purity) as a white solid and it was confirmed by HNMR and LCMS

### Mass Found:

Retention time: 0.310 min, (M+H)=489.0
Retention time: 1.434 min, (M+H)=489.2

### NMR Data:

1H NMR (400 MHz, DMSO-d6) δ=12.46 (s, 1H), 11.34-11.12 (m, 1H), 10.48 (s, 1H), 7.89-7.86 (m, 1H), 7.75 (d, J=2.0 Hz, 1H), 7.67 (d, J=8.8 Hz, 1H), 7.55-7.52 (m, 1H), 7.22-7.17 (m, 2H), 7.12-7.08 (m, 1H), 4.77-4.55 (m, 4H), 3.68-3.49 (m, 2H), 2.77-2.72 (m, 3H), 2.25-2.07 (m, 2H)

### Example 39. Synthesis of [[5-acetyl-N-[6-(2-thienylsulfonylamino)-1,3-benzothiazol-2-yl]-4,6,7,8-tetrahydropyrazolo[1,5-a][1,4]diazepine-2-carboxamide]] (Intermediate 39)

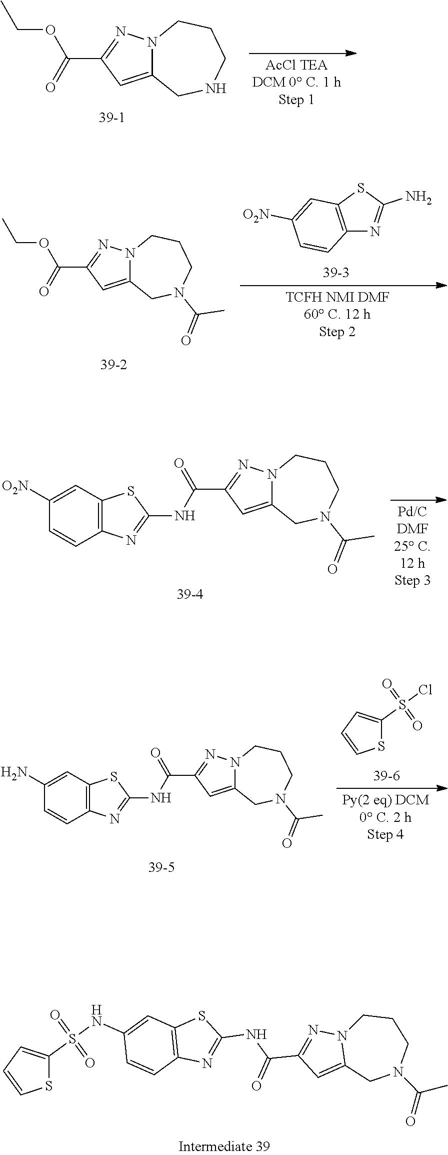

### Step 1: Synthesis of Intermediate 39-2

Detailed Synthetic Procedure: To a solution of Intermediate 39-1 (500 mg, 2.39 mmol, 1 eq) in DCM (5 mL) was added acetyl chloride (375.15 mg, 4.78 mmol, 341.04 uL, 2 eq) and TEA (1.21 g, 11.95 mmol, 1.66 mL, 5 eq). The mixture was stirred at 25° C. for 1 hr. LCMS (EC4311-83-P1A1) showed Intermediate 39-1 was consumed completely and one main peak with desired mass was detected. TLC (EA:MeOH=5:1) indicated Intermediate 39-1 (Rf=0.0) was consumed completely and one new spot (Rf=0.8) formed. The reaction was clean according to TLC. The reaction was diluted with H2O (5 mL), extracted with DCM 30 mL (10 mL*3). The organic phase was washed with saturated aqueous NaCl (5 mL). Then dried over Na2SO4, filtered and concentrated under reduced pressure to give a residue. The residue was purified by flash silica gel chromatography (ISCO®; 20 g SepaFlash® Silica Flash Column, Eluent of 0˜60% Ethyl acetate/MeOH ether gradient @ 40 mL/min). The eluent was concentrated to afford product. Intermediate 39-2 (500 mg, 1.99 mmol, 83.27% yield, N/A purity) was obtained as a white solid. It was confirmed by LCMS, HNMR, VT-NMR (80° C.).

### Mass Found

LCMS: Retention time: 0.282 min, (M+H)=252.1

LCMS: Retention time: 0.269 min, (M+H)=251.8

### NMR Data

1H NMR (400 MHz, DMSO-d6) δ=6.98-6.43 (m, 1H), 4.69-4.56 (m, 2H), 4.53-4.45 (m, 2H), 4.24-4.21 (m, 2H), 3.81-3.70 (m, 2H), 2.01-1.97 (m, 3H), 1.90-1.84 (m, 1H), 1.79-1.71 (m, 1H), 1.28-1.24 (m, 3H).

1H NMR (400 MHz, DMSO-d6) δ=6.83-6.49 (m, 1H), 4.72-4.58 (m, 2H), 4.54-4.42 (m, 2H), 4.28-4.23 (m, 2H), 3.81-3.72 (m, 2H), 2.00 (s, 3H), 1.91 (s, 1H), 1.83 (br d, J=7.2 Hz, 1H), 1.29 (t, J=7.2 Hz, 3H)

### Step 2: Synthesis of Intermediate 39-4

Detailed Synthetic Procedure: To a solution of Intermediate 39-2 (60 mg, 238.78 umol, 1 eq) and Intermediate 39-3 (93.22 mg, 477.55 umol, 2 eq) in THE (0.5 mL) was added 3,4,6,7,8,9-hexahydro-2H-pyrimido[1,2-a]pyrimidine (66.48 mg, 477.55 umol, 2 eq). The mixture was stirred at 80° C. for 12 hr. LCMS (EC4311-91-P1A2) showed a new peaks were shown on LCMS and 34% of desired compound was detected. TLC (DCM:MeOH=10:1) indicated Intermediate 39-2 (Rf=0.46) was consumed completely and one new spot (Rf=0.72) formed. The reaction was clean according to TLC. The reaction was cooled to room temperature, which was diluted with H2O (5 mL), extracted with DCM 30 mL (10 mL*3). The organic phase was washed with saturated aqueous NaCl (5 mL). Then dried over Na2SO4, filtered and concentrated under reduced pressure to give a residue. The residue was purified by flash silica gel chromatography (ISCO®; 20 g SepaFlash® Silica Flash Column, Eluent of 0˜70% Ethyl acetate/Petroleum ether gradient @ 30 mL/min). The eluent was concentrated to afford product. Intermediate 39-4 (80 mg, 199.79 umol, 83.67% yield) was obtained as a yellow solid. It was confirmed by HNMR.

### Mass Found

LCMS: Retention time: 0.633 min, (M+H)=401.0

### NMR Data

1H NMR (400 MHz, DMSO-d6) δ=12.80 (br s, 1H), 9.13-9.05 (m, 1H), 8.31-8.28 (m, 1H), 7.93 (d, J=8.8 Hz, 1H), 7.23-6.93 (m, 1H), 4.78-4.64 (m, 2H), 4.63-4.54 (m, 2H), 3.80 (br d, J=4.8 Hz, 2H), 2.03 (d, J=12.8 Hz, 3H), 1.98-1.76 (m, 2H)

### Step 3: Synthesis of Intermediate 39-5

Detailed Synthetic Procedure: To a solution of Intermediate 39-4 (80 mg, 199.79 umol, 1 eq) in DMF (3 mL) was added Pd/C (100 mg, 10% purity) under N2 atmosphere. The suspension was degassed under vacuum and purged with H2 several times. The mixture was stirred under H2 (15 Psi) at 25° C. for 12 hr. LCMS (EC4466-134-P1A2) showed Intermediate 39-4 was consumed and desired mass was detected. The reaction was diluted with MeOH 5 mL*3 (15 mL), filtered and concentrated under reduced pressure to give a residue. The crude product Intermediate 39-5 (60 mg, crude) was a yellow solid and it was used into the next step without further purification.

### Mass Found

LCMS: Retention time: 0.321 min, (M+H)=371.0

### Step 4: Synthesis of Intermediate 39

Detailed Synthetic Procedure: To a solution of Intermediate 39-5 (60 mg, 161.97 umol, 1 eq) in DCM (1 mL) was added Py (25.62 mg, 323.95 umol, 26.15 uL, 2 eq) and Intermediate 39-6 (35.50 mg, 194.37 umol, 1.2 eq). The mixture was stirred at 0° C. for 2 hr. showed Intermediate 39-5 was consumed and desired mass was detected. The reaction was concentrated under reduced pressure to give a residue. The residue was diluted with MeOH (2 mL). The residue was purified by prep-HPLC (column: Phenomenex luna C18 150*25 mm*10 um; mobile phase: [water (NH4HCO3)-ACN]; B %: 22%-52%, 8 min). The eluent was lyophilized to give product. Compound 1 (14 mg, 26.54 umol, 16.38% yield, 97.92% purity) was obtained as a yellow solid. It was confirmed by LCMS, HNMR, and VT-NMR (80° C.).

### Mass Found

LCMS: Retention time: 0.628 min, (M+H)=516.9

LCMS: Retention time: 0.342 min, (M+H)=517.0

### NMR Data

1H NMR (400 MHz, DMSO-d6) δ=12.40-12.10 (m, 1H), 10.64-10.26 (m, 1H), 7.87 (d, J=4.8 Hz, 1H), 7.77-7.70 (m, 1H), 7.69-7.61 (m, 1H), 7.57-7.48 (m, 1H), 7.19-7.18 (m, 1H), 7.13-7.08 (m, 1H), 7.07-6.88 (m, 1H), 4.74-4.62 (m, 2H), 4.55 (d, J=7.6 Hz, 2H), 3.79 (d, J=4.0 Hz, 2H), 2.06-1.99 (m, 3H), 1.97-1.76 (m, 2H).

1H NMR (400 MHz, DMSO-d6) δ=7.83 (d, J=5.2 Hz, 1H), 7.71 (d, J=1.6 Hz, 1H), 7.64 (d, J=8.8 Hz, 1H), 7.56-7.46 (m, 1H), 7.22-7.21 (m, 1H), 7.09 (t, J=4.4 Hz, 1H), 7.05-6.84 (m, 1H), 4.74-4.64 (m, 2H), 4.58-4.52 (m, 2H), 3.82-3.77 (m, 2H), 2.03 (br s, 3H), 2.00-1.79 (m, 2H).

### Example 40. Synthesis of [[5-but-3-ynyl-N-[6-(2-thienylsulfonylamino)-1,3-benzothiazol-2-yl]-4,6,7,8-tetrahydropyrazolo[1,5-a][1,4]diazepine-2-carboxamide]] (Intermediate 40)

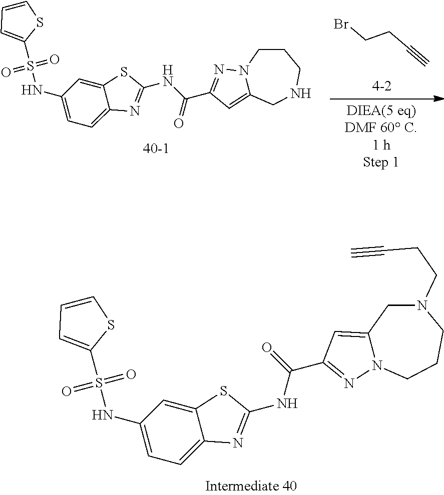

### Step 1: Synthesis of Intermediate 40

Detailed Synthetic Procedure: To a solution of Intermediate 40-1 (50 mg, 97.84 umol, 1 eq, HCl) and Intermediate 40-2 (39.03 mg, 293.52 umol, 3 eq) in DMF (0.5 mL) was added DIEA (63.23 mg, 489.20 umol, 85.21 uL, 5 eq). The mixture was stirred at 60° C. for 1 hr. The showed the desired mass was detected. The mixture was filtered to give a residue. The residue was diluted with MeOH (2 mL) and purified by prep-HPLC (column: Phenomenex luna C18 150*25 mm*10 um; mobile phase: [water(FA)-ACN];B %: 8%-38%, 10 min), the eluent was lyophilized to give Compound 1 (10.2 mg, 18.40 umol, 18.81% yield, 95.024% purity) as brown solid which was confirmed by LCMS and HNMR.

### Mass Found

LCMS: Retention time: 0.344 min, (M+H)=527.1

LCMS: Retention time: 0.343 min, (M+H)=527.2

1H NMR (400 MHz, DMSO-d6) δ=7.83 (d, J=4.4 Hz, 1H), 7.72 (d, J=2.0 Hz, 1H), 7.66 (d, J=8.8 Hz, 1H), 7.53 (d, J=3.2 Hz, 1H), 7.23-7.15 (m, 2H), 7.09 (t, J=4.4 Hz, 1H), 4.72 (s, 2H), 4.58 (s, 2H), 3.63 (s, 2H), 3.20 (s, 2H), 3.03 (s, 1H), 2.77-2.68 (m, 2H), 2.15 (s, 2H).

### Example 41. Synthesis of 2-((3-((2-fluoro-9H-fluoren-9-ylidene)methyl)pyridin-2-yl)oxy)ethan-1-amine hydrochloride (Intermediate 41)

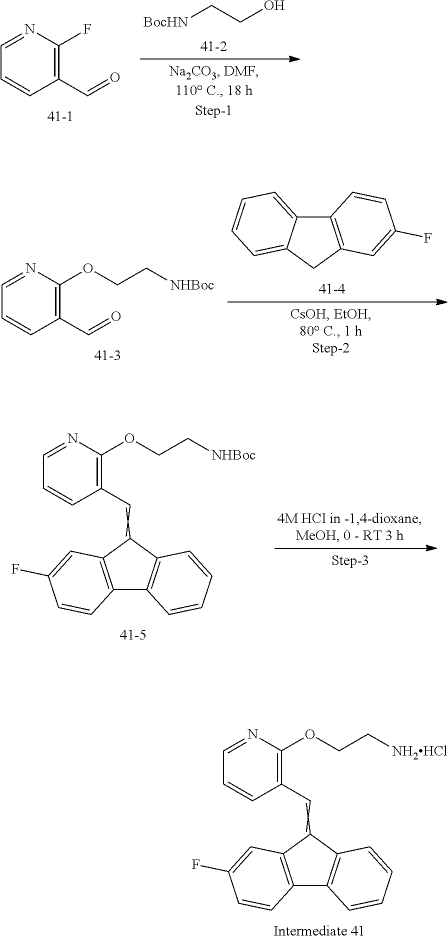

### Step 1: tert-butyl (2-((3-formylpyridin-2-yl)oxy)ethyl)carbamate (41-3)

To a solution of 2-fluoronicotinaldehyde (41-1, 10 g, 80 mmol) and tert-butyl (2-hydroxyethyl)carbamate (41-2, 25.8 g, 160 mmol) in DMF (100 mL), Na₂CO₃ (17.12 g, 160 mmol) was added at RT and the reaction mixture was heated to 110° C. for 24 h. After completion (monitored by LCMS), the reaction mixture was cooled to RT and diluted with water (200 mL) and extracted with EtOAc (2×200 mL). The combined organic extract was washed with brine (50 mL), dried over anhydrous sodium sulphate, filtered, and concentrated under reduced. The crude residue was purified by flash column chromatography (silica-gel, 230-400mesh size) using EtOAc-hexane (30 to 60%) as an eluent to obtain the title compound (5.7 g, 23% yield) as an off-white solid. ¹H NMR (400 MHz, CHLOROFORM-d) δ=10.45-10.37 (m, 1H), 8.38 (dd, J=2.0, 4.9 Hz, 1H), 8.15 (dd, J=2.1, 7.5 Hz, 1H), 7.08-7.03 (m, 1H), 5.00-4.91 (m, 1H), 4.56 (t, J=5.3 Hz, 2H), 3.66-3.59 (m, 2H), 1.47 (s, 9H). LCMS: (Method C) 267.1 (M+H)⁺, Rt. 2.46 min, 87.44% (Max).

### Step 2: tert-butyl (2-((3-((2-fluoro-9H-fluoren-9-ylidene)methyl)pyridin-2-yl)oxy)ethyl)carbamate (41-5)

To a stirred solution of 2-fluoro-9H-fluorene (41-4, 2.07 g, 11.27 mmol) in ethanol (30 mL) at RT were added CsOH·H₂O (0.315 g, 1.88 mmol) followed by tert-butyl (2-((3-formylpyridin-2-yl)oxy)ethyl)carbamate (41-3, 2.5 g, 9.39 mmol), and the reaction mixture was heated to 60° C. for 1.5 h. After cooling, the precipitated solid was collected by filtration, washed with cold EtOH (5 mL), and dried under vacuum to get the title compound (5, 4.1 g, 92% Yield) as a pale-yellow solid. ¹H NMR (400 MHz, CHLOROFORM-d) δ=8.28-8.21 (m, 1H), 7.98-7.91 (m, 1H), 7.86-7.70 (m, 1H), 7.70-7.64 (m, 2H), 7.60-7.47 (m, 2H), 7.45-7.30 (m, 2H), 7.24-7.00 (m, 3H), 4.92-4.80 (m, 1H), 4.55-4.47 (m, 2H), 3.58-3.49 (m, 2H), 1.42 (s, 9H). LCMS: (Method A) 433.3 (M+H), Rt. 2.869 min, 92.88% (Max).

### Step 3: 2-((3-((2-fluoro-9H-fluoren-9-ylidene)methyl)pyridin-2-yl)oxy)ethan-1-amine hydrochloride (Intermediate 41)

To a stirred solution of tert-butyl (2-((3-((2-fluoro-9H-fluoren-9-ylidene)methyl)pyridin-2-yl)oxy)ethyl)carbamate (41-5, 0.15 g, 0.35 mmol) in MeOH (2 mL) was added HCl in 1,4-dioxane (0.87 mL, 4 M, 3.47 mmol) at 0° C. and the reaction mixture was stirred at RT for 18 h. After completion (monitored by LCMS), the reaction mixture was concentrated under vacuum to get the title compound (0.125 g, 95% yield) as a pale-yellow solid. ¹H NMR (400 MHz, DMSO-d₆) δ=8.33-8.27 (m, 1H), 8.24-8.08 (m, 4H), 8.06-7.84 (m, 4H), 7.55-7.35 (m, 2H), 7.30-7.12 (m, 3H), 4.65-4.49 (m, 2H), 3.30-3.20 (m, 2H). LCMS: (Method A) 333.2 (M+H), Rt. 1.72 min, 97.34% (Max).

### Example 36. Synthesis of (R)-3-(6-(1-(2,2-difluorobenzo[d][1,3]dioxol-5-yl)cyclopropane-1-carboxamido)-3-methylpyridin-2-yl)-N-(15-(3-((4-hydroxy-1-(3-phenylbutanoyl)piperidin-4-yl)methyl)-4-oxo-3,4-dihydrothieno[3,2-d]pyrimidin-7-yl)-3,6,9,12-tetraoxapentadec-14-yn-1-yl)benzamide (Compound 1)

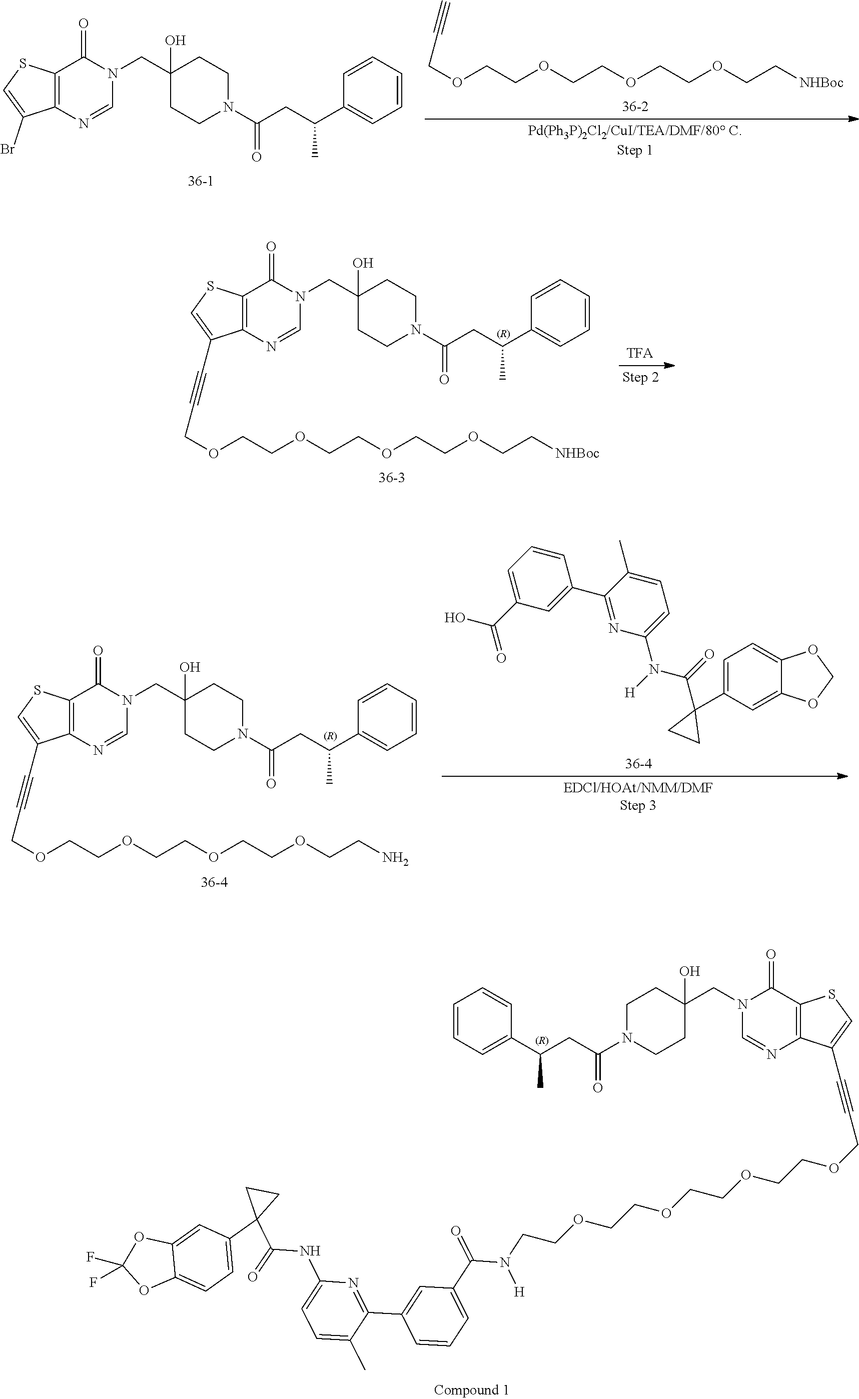

### Example 37. Synthesis of (R)-3-(6-(1-(2,2-difluorobenzo[d][1,3]dioxol-5-yl)cyclopropane-1-carboxamido)-3-methylpyridin-2-yl)-N-(1-((3-((4-hydroxy-1-(3-phenylbutanoyl)piperidin-4-yl)methyl)-4-oxo-3,4-dihydrothieno[3,2-d]pyrimidin-7-yl)amino)-6,9,12-trioxa-3-azatetradecan-14-yl)benzamide (Compound 2)

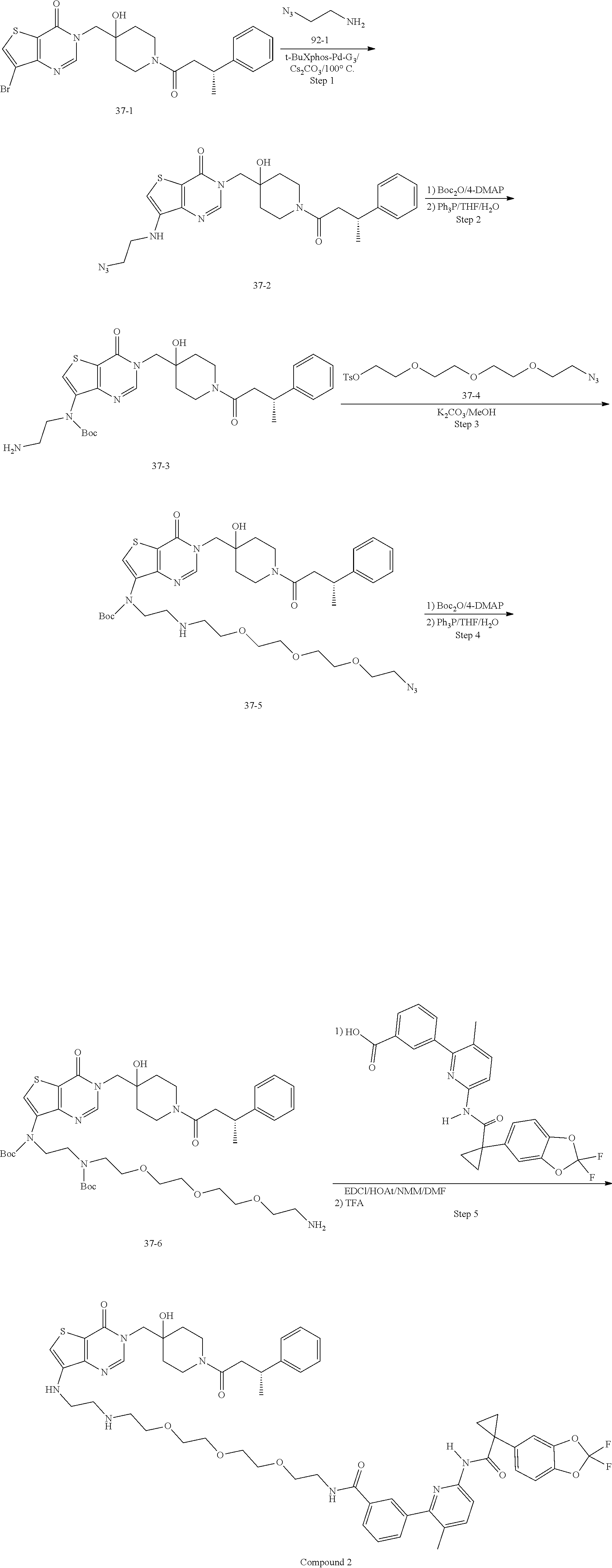

### Example 38. Synthesis of (R)-3-(6-(1-(2,2-difluorobenzo[d][1,3]dioxol-5-yl)cyclopropane-1-carboxamido)-3-methylpyridin-2-yl)-N-(1-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)phenyl)-3-oxo-5,8,11-trioxa-2-azatridecan-13-yl)benzamide (Compound 3)

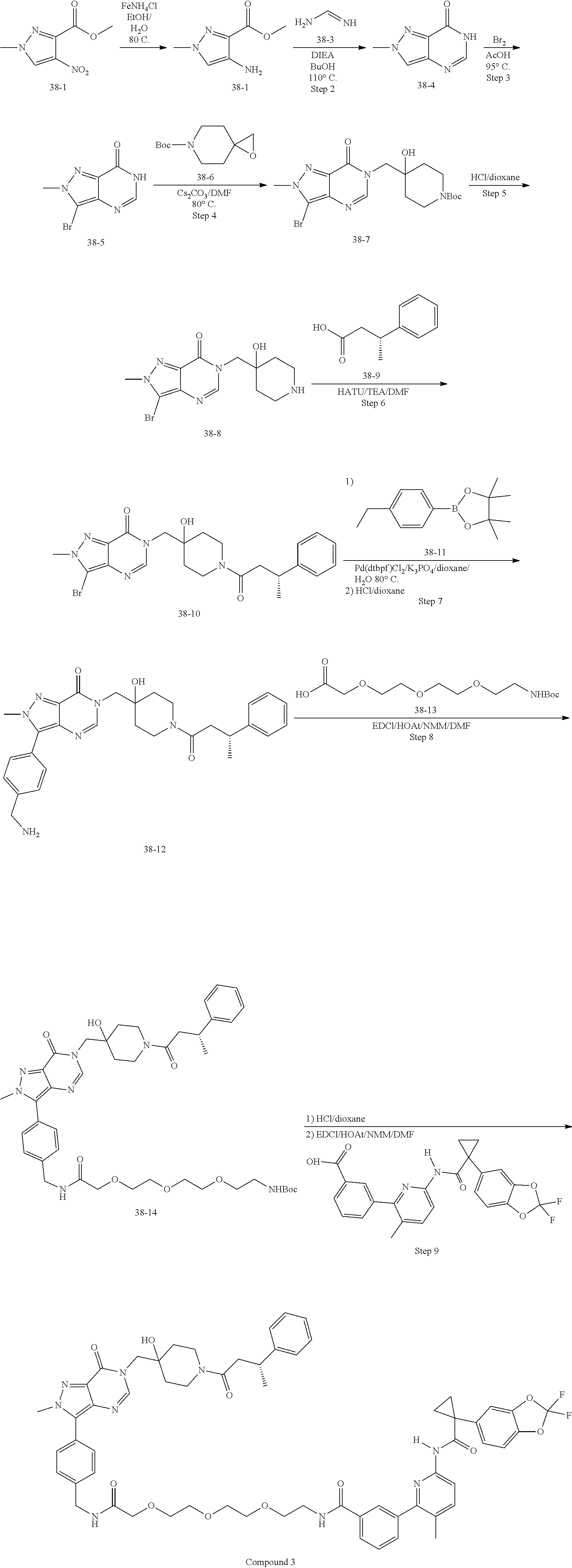

### Example 39. Synthesis of (R)-3-(6-(1-(2,2-difluorobenzo[d][1,3]dioxol-5-yl)cyclopropane-1-carboxamido)-3-methylpyridin-2-yl)-N-(15-(3-((4-hydroxy-1-(3-phenylbutanoyl)piperidin-4-yl)methyl)-4-oxo-3,4-dihydroquinazolin-7-yl)-11-oxo-3,6,9-trioxa-12-azapentadec-14-yn-1-yl)benzamide (Compound 4)

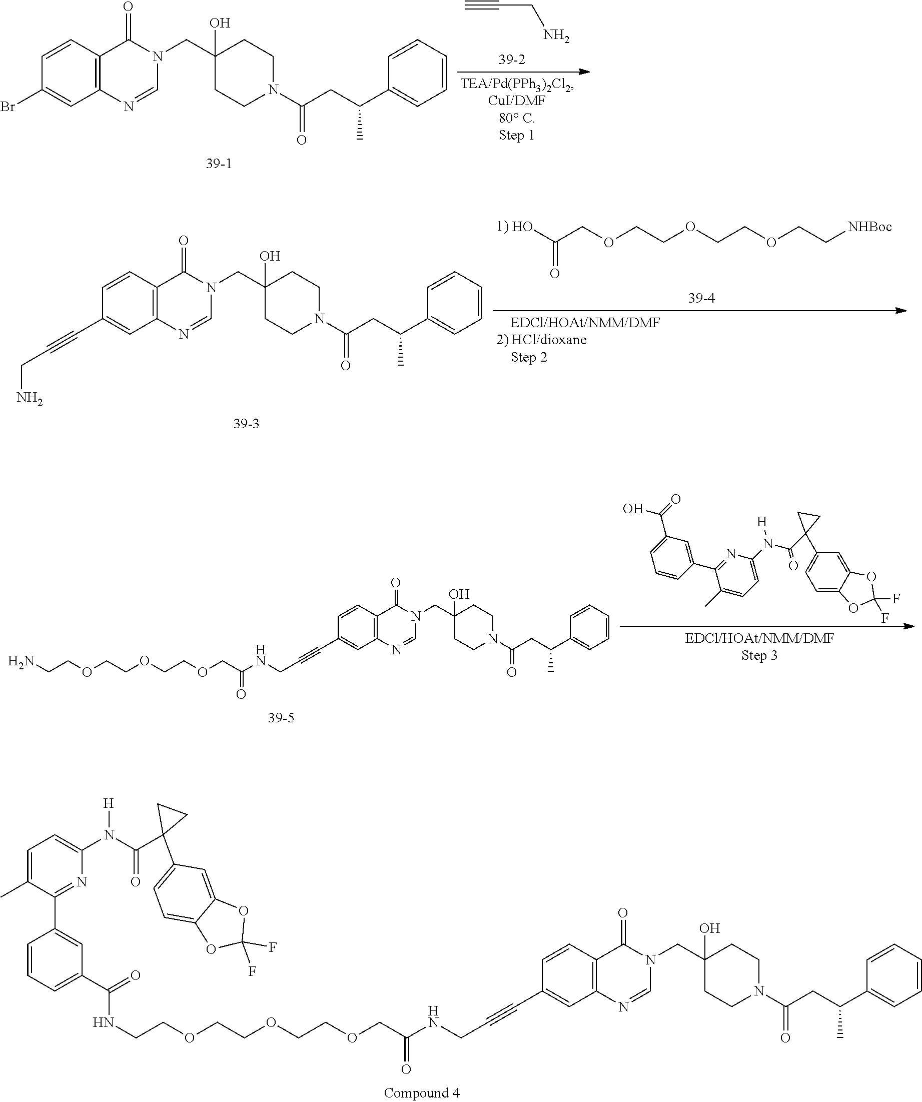

### Example 40. Synthesis of (R)-3-(6-(1-(2,2-difluorobenzo[d][1,3]dioxol-5-yl)cyclopropane-1-carboxamido)-3-methylpyridin-2-yl)-N-(1-(4-(2-((1-((4-hydroxy-1-(3-phenylbutanoyl)piperidin-4-yl)methyl)-6-oxo-1,6-dihydropyrimidin-4-yl)amino)ethyl)piperazin-1-yl)-4-oxo-6,9,12-trioxa-3-azatetradecan-14-yl)benzamide (Compound 5)

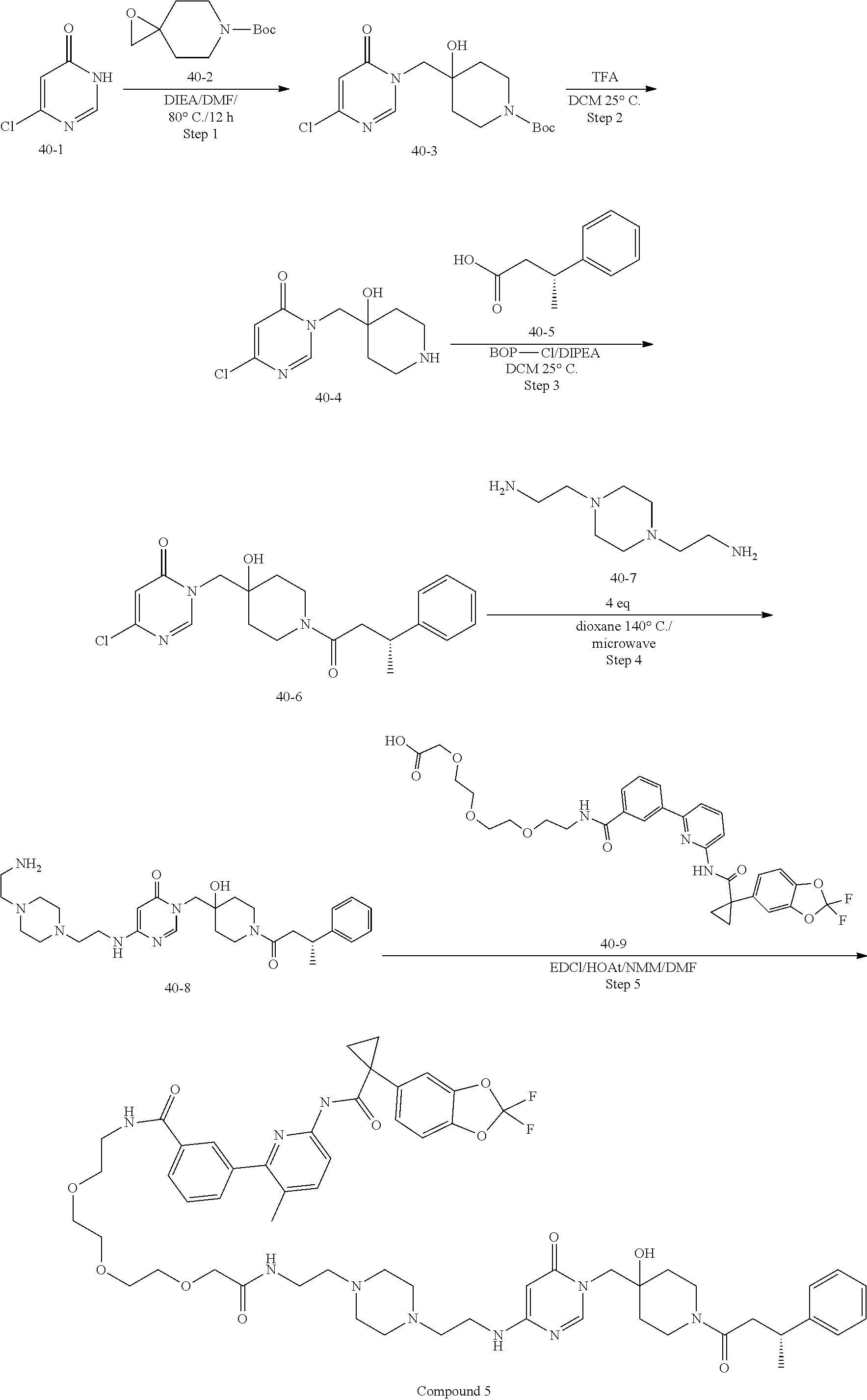

### Example 41. Synthesis of N-(2-(2-(2-(4-(2-amino-4-ethyl-5-(1H-indazol-5-yl)pyridin-3-yl)phenoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 6)

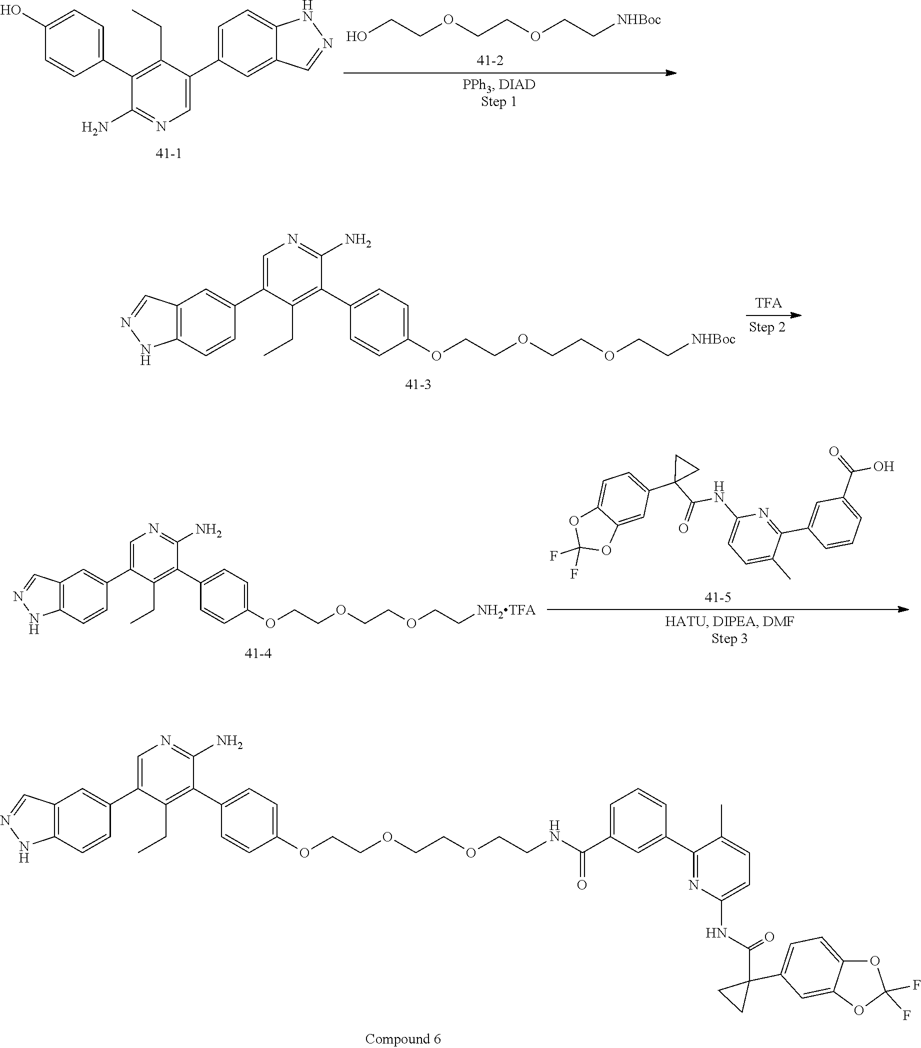

### Example 42. Synthesis of N-(2-(4-((2-(2-(4-(2-amino-4-ethyl-5-(1H-indazol-5-yl)pyridin-3-yl)phenoxy)ethoxy)ethoxy)methyl)-1H-1,2,3-triazol-1-yl)ethyl)-3-(6-(1-(2,2-difluorobenzo[d][1,3]dioxol-5-yl)cyclopropane-1-carboxamido)-3-methylpyridin-2-yl)benzamide (Compound 7)

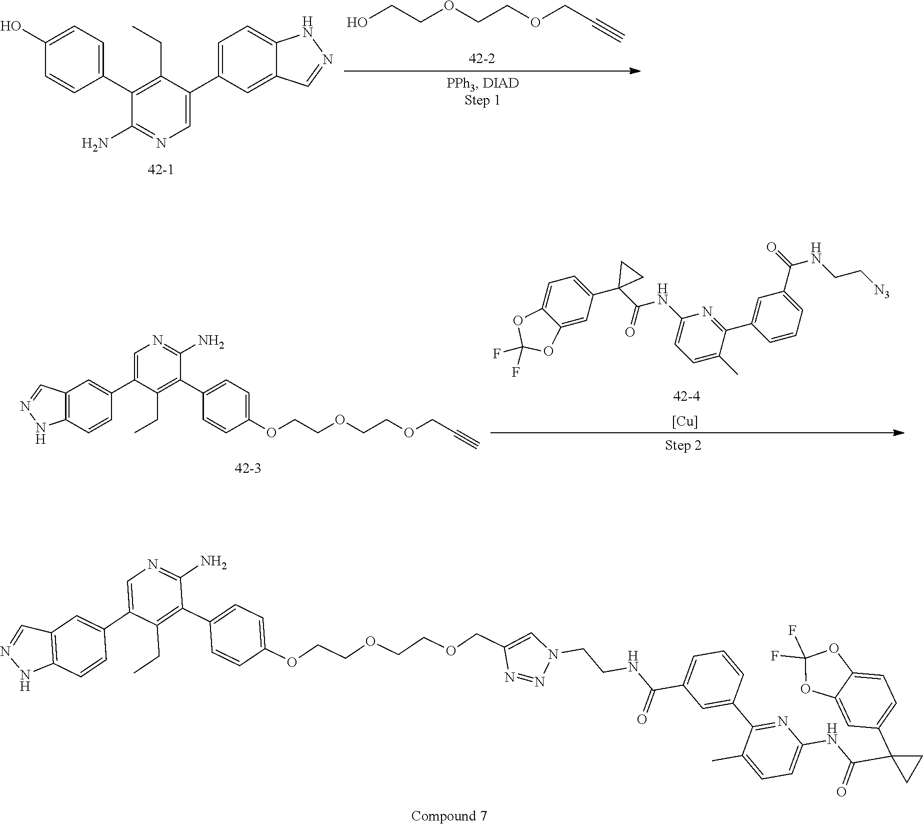

### Example 43. Synthesis of N-(2-(2-(3-((6′-amino-4′-ethyl-5′-(4-hydroxyphenyl)-[3,3′-bipyridin]-6-yl)amino)-3-oxopropoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 8)

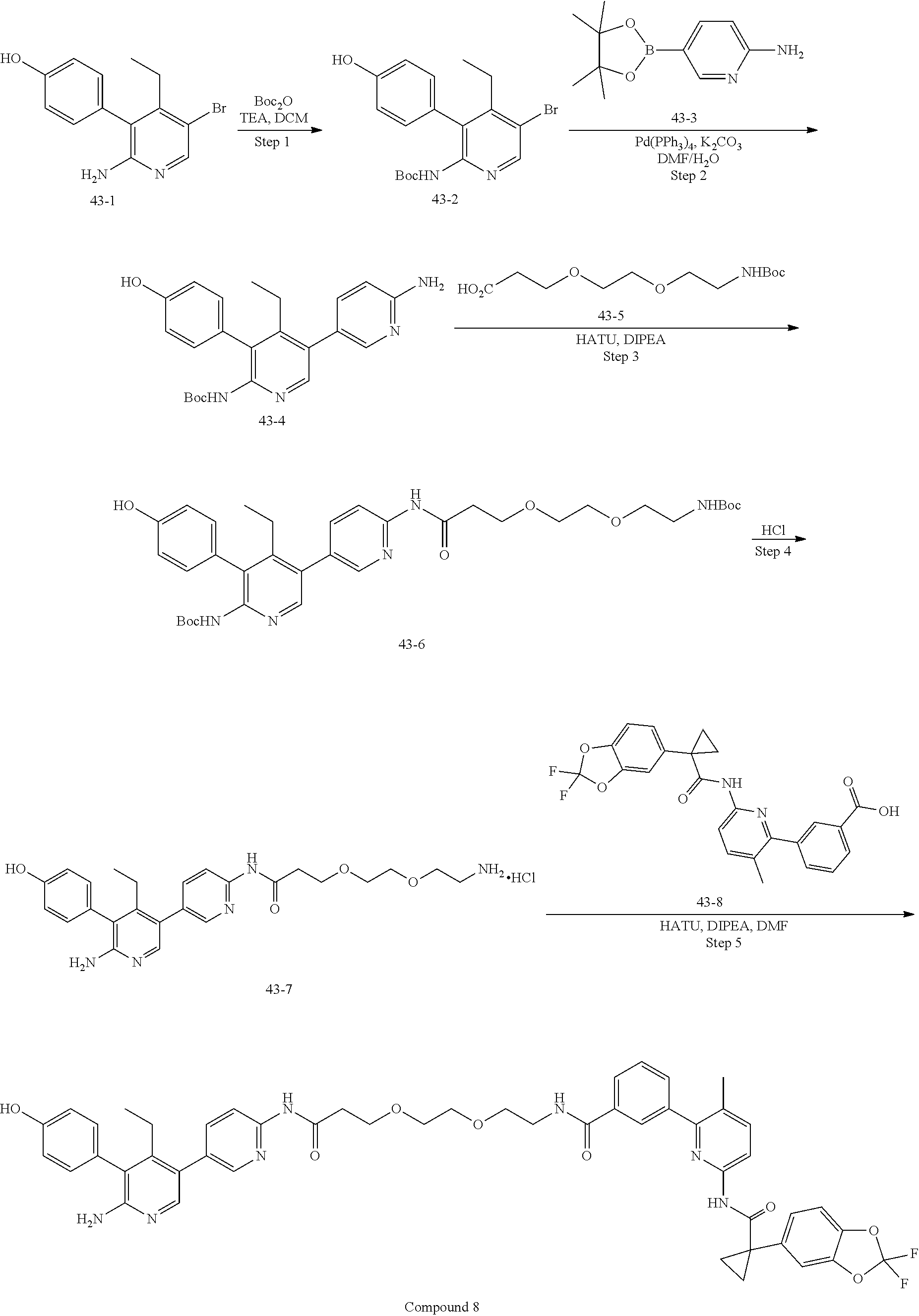

### Example 44. Synthesis of N-(2-(4-((2-(3-((6′-amino-4′-ethyl-5′-(4-hydroxyphenyl)-[3,3′-bipyridin]-6-yl)amino)-3-oxopropoxy)ethoxy)methyl)-1H-1,2,3-triazol-1-yl)ethyl)-3-(6-(1-(2,2-difluorobenzo[d][1,3]dioxol-5-yl)cyclopropane-1-carboxamido)-3-methylpyridin-2-yl)benzamide (Compound 9)

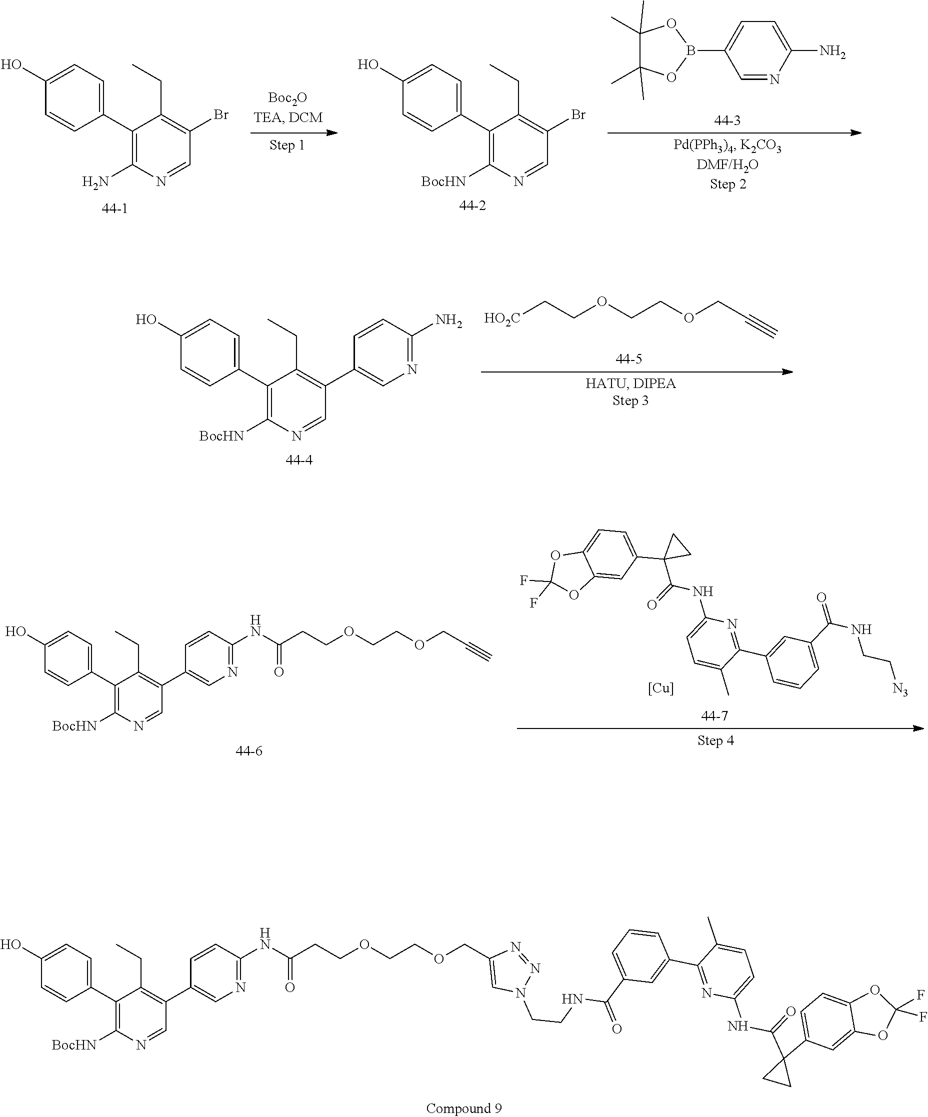

### Example 45. Synthesis of N-(2-(2-(2-((3R,4S)-3-(4-chloro-2-(2-((2,4-dioxothiazolidin-3-yl)methyl)thieno[3,2-b]pyridin-7-yl)-6-methylphenoxy)-4-fluoropyrrolidin-1-yl)-2-oxoethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 10)

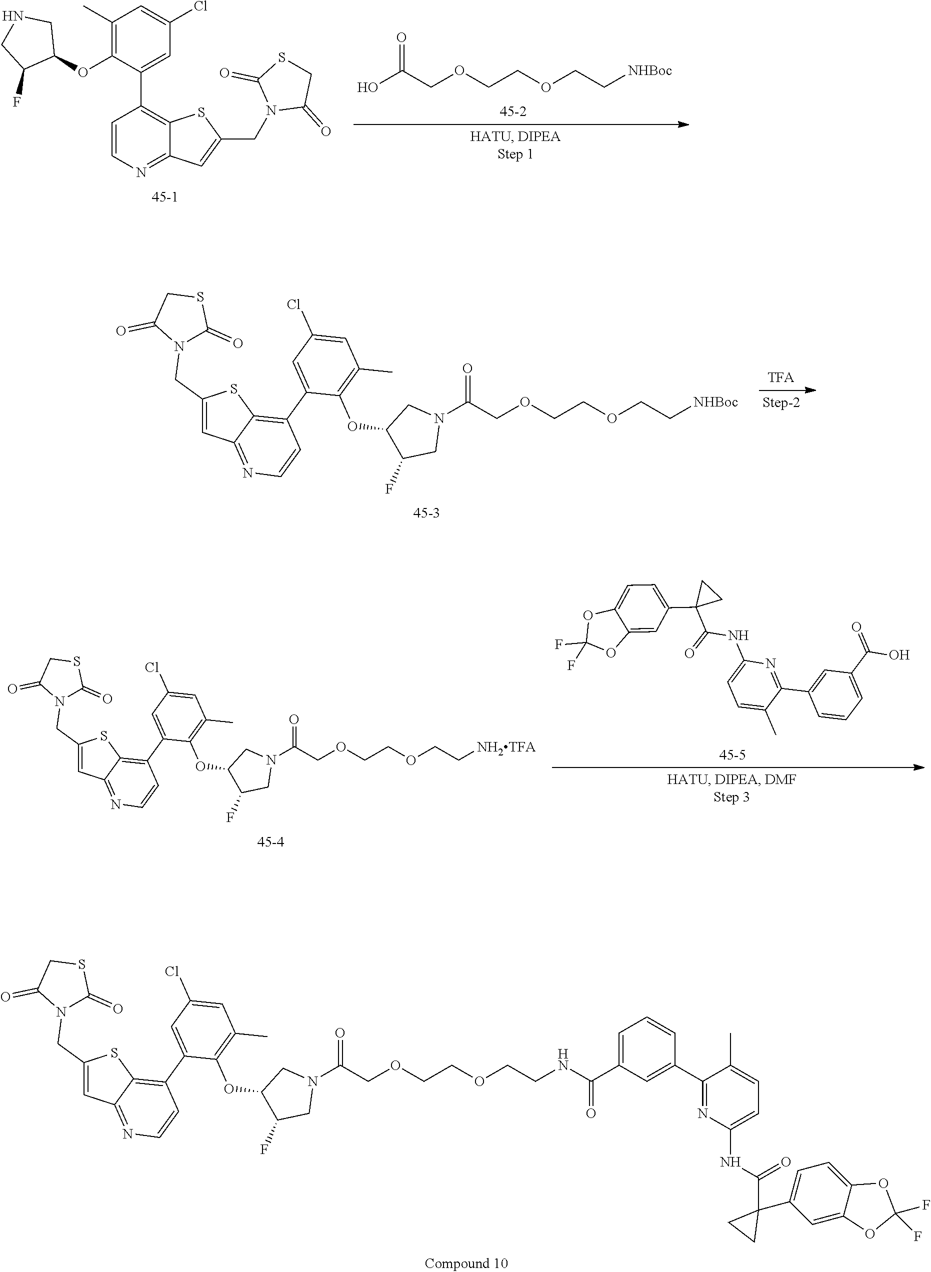

### Example 46. Synthesis of N-(2-(4-((2-(3-((3R,4S)-3-(4-chloro-2-(2-((2,4-dioxothiazolidin-3-yl)methyl)thieno[3,2-b]pyridin-7-yl)-6-methylphenoxy)-4-fluoropyrrolidin-1-yl)-3-oxopropoxy)ethoxy)methyl)-1H-1,2,3-triazol-1-yl)ethyl)-3-(6-(1-(2,2-difluorobenzo[d][1,3]dioxol-5-yl)cyclopropane-1-carboxamido)-3-methylpyridin-2-yl)benzamide (Compound 11)

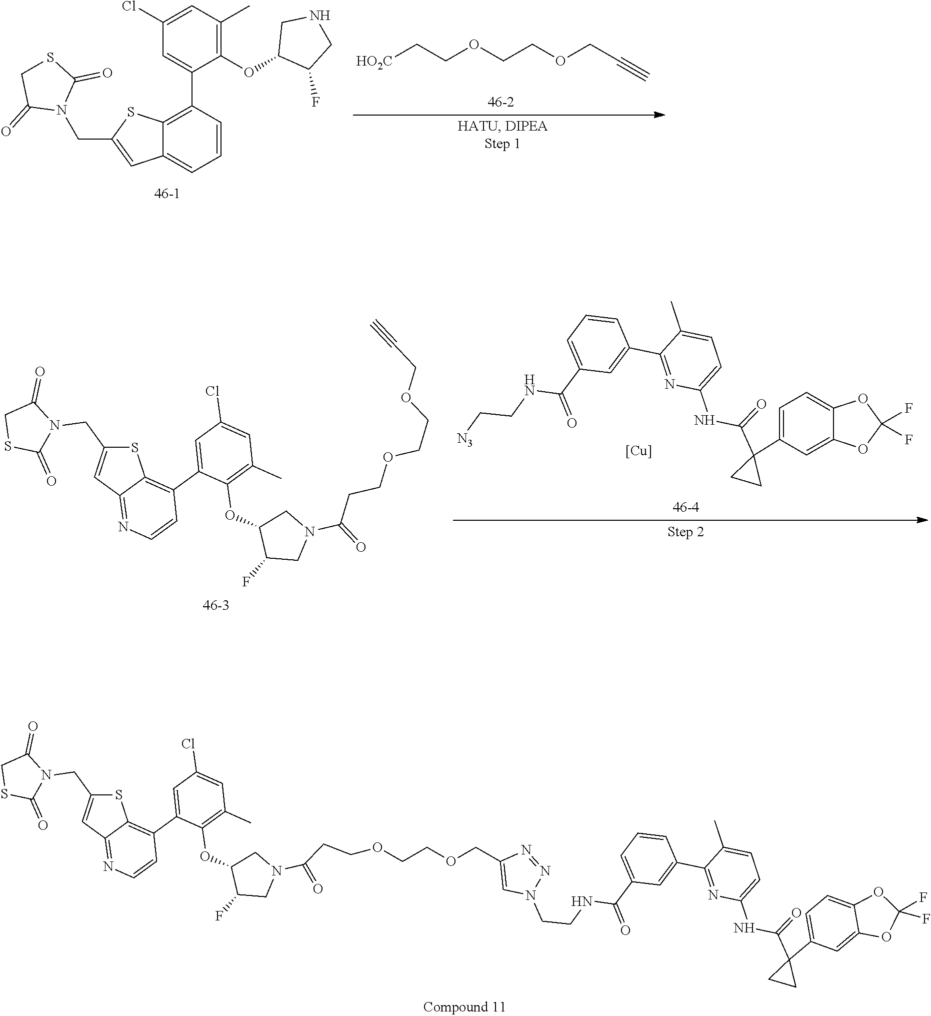

### Example 47. Synthesis of (R)—N-(2-(2-(2-(4-(5-chloro-7-(2-((2,5-dioxopyrrolidin-1-yl)methyl)thieno[3,2-b]pyridin-7-yl)-2,3-dihydrobenzofuran-2-carbonyl)piperazin-1-yl)-2-oxoethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 12)

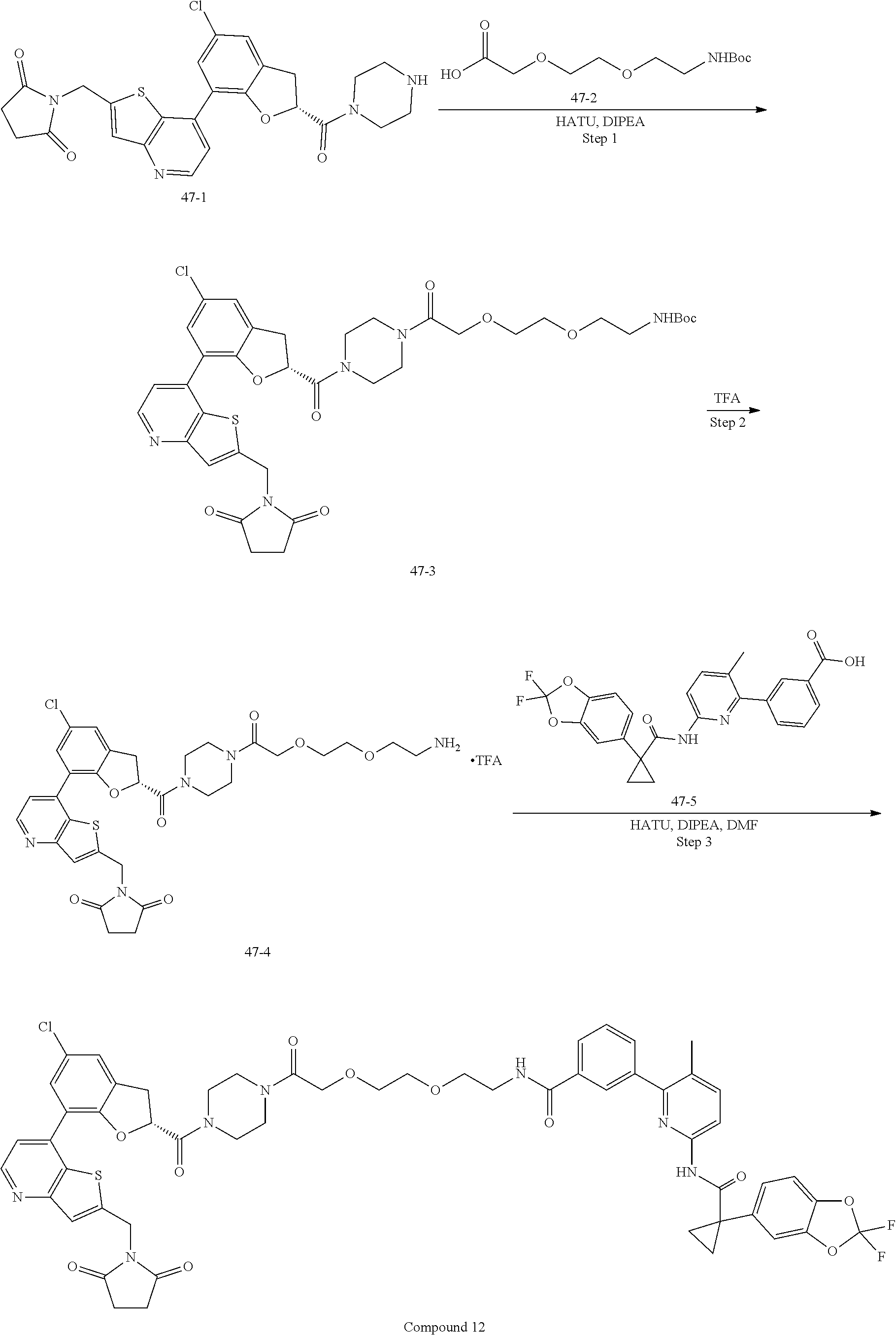

### Example 48. Synthesis of (R)—N-(2-(4-((2-(3-(4-(5-chloro-7-(2-((2,5-dioxopyrrolidin-1-yl)methyl)thieno[3,2-b]pyridin-7-yl)-2,3-dihydrobenzofuran-2-carbonyl)piperazin-1-yl)-3-oxopropoxy)ethoxy)methyl)-1H-1,2,3-triazol-1-yl)ethyl)-3-(6-(1-(2,2-difluorobenzo[d][1,3]dioxol-5-yl)cyclopropane-1-carboxamido)-3-methylpyridin-2-yl)benzamide (Compound 13)

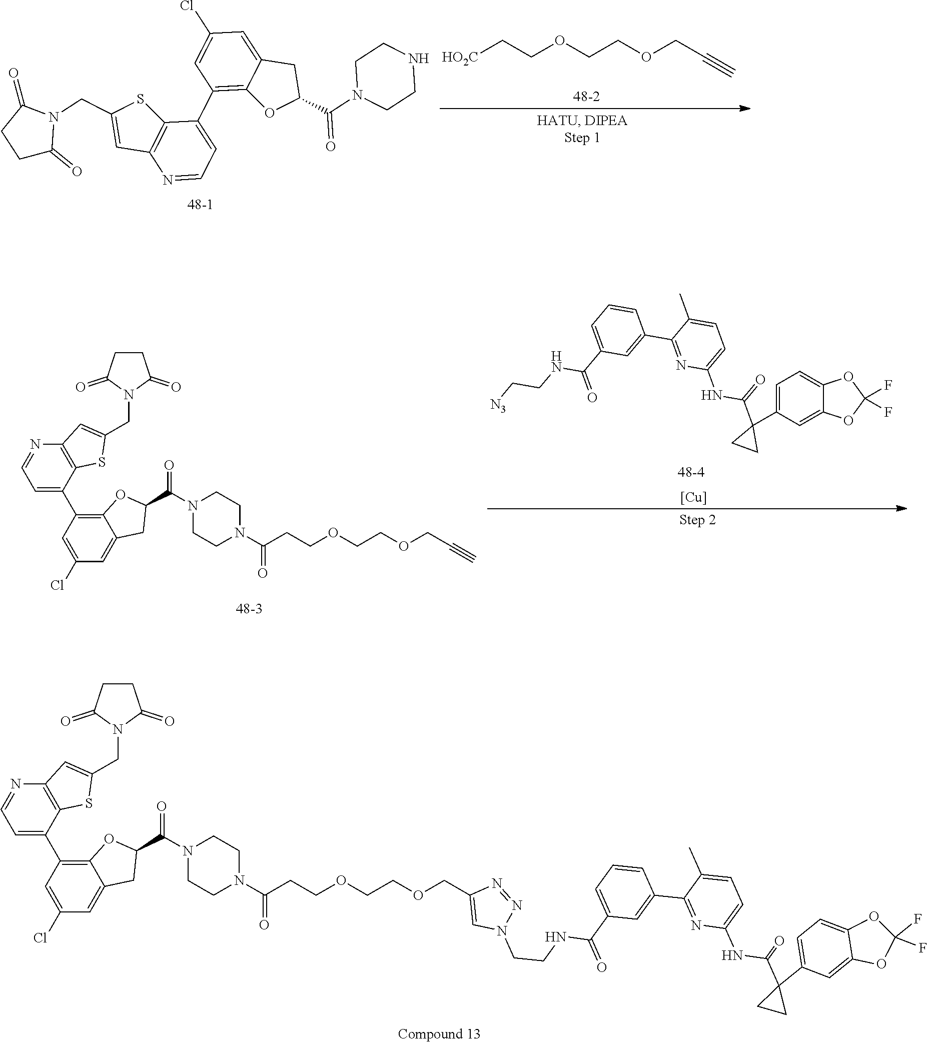

### Example 49. Synthesis of (R)-3-(6-(1-(2,2-difluorobenzo[d][1,3]dioxol-5-yl)cyclopropane-1-carboxamido)-3-methylpyridin-2-yl)-N-(2-(2-(2-((2-((3-((4-hydroxy-1-(2-methyl-3-phenylpropanoyl)piperidin-4-yl)methyl)-4-oxo-3,4-dihydroquinazolin-7-yl)amino)ethyl)amino)-2-oxoethoxy)ethoxy)ethyl)benzamide (Compound 14)

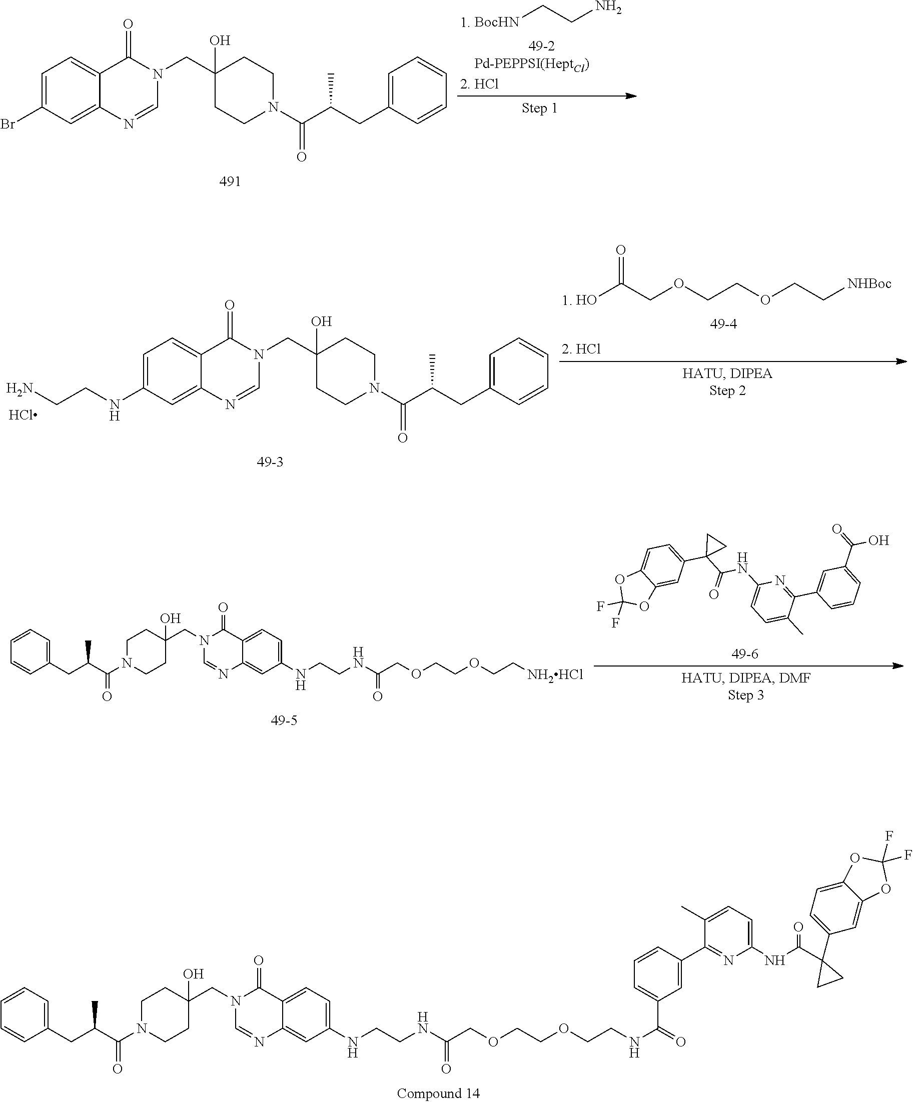

### Example 50. Synthesis of 6′-amino-5′-(4-(2-(2-(2-(3-(6-(1-(2,2-difluorobenzo[d][1,3]dioxol-5-yl)cyclopropane-1-carboxamido)-3-methylpyridin-2-yl)benzamido)ethoxy)ethoxy)ethoxy)phenyl)-4′-ethyl-N-methyl-[3,3′-bipyridine]-6-carboxamide (Compound 15)

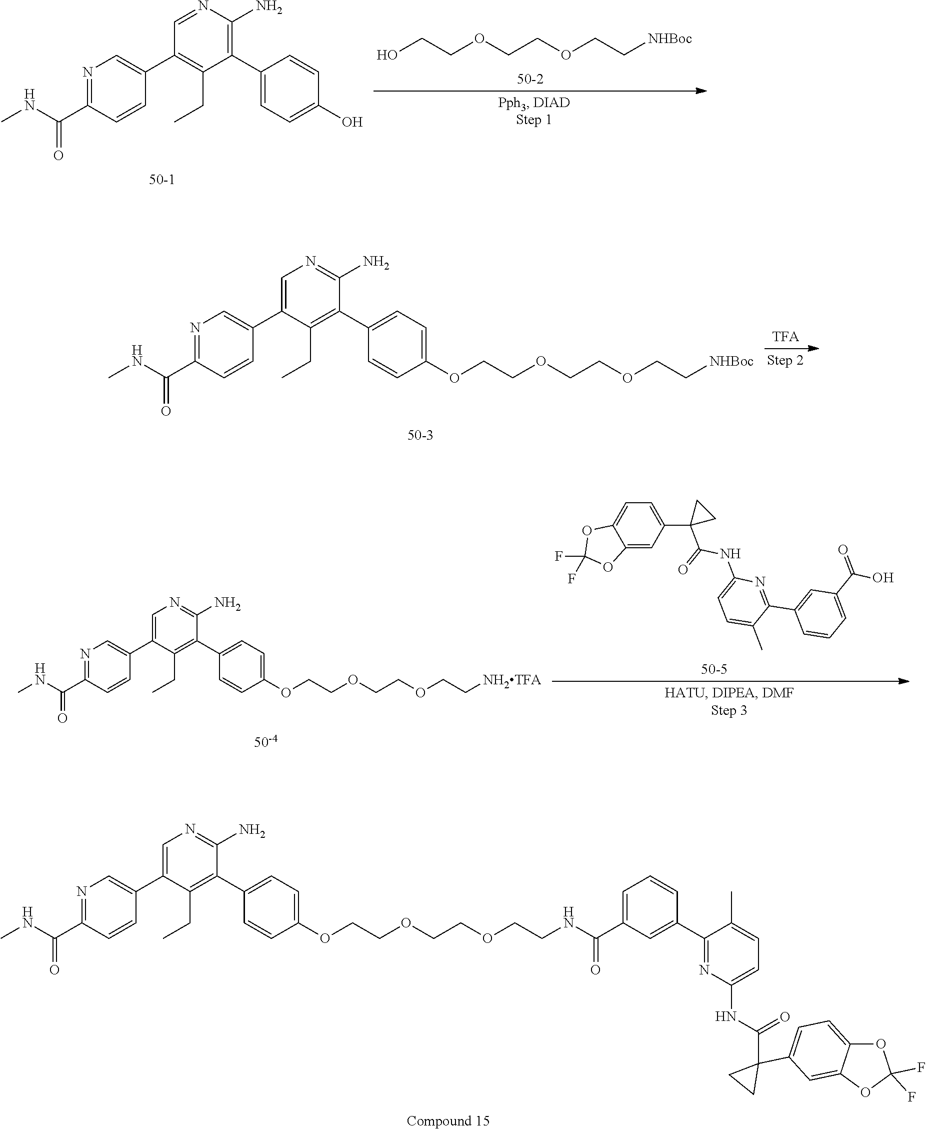

### Example 51. Synthesis of methyl 3-chloro-4-((4-((3-(4-((3-(2-((1-(2-(3-(6-(1-(2,2-difluorobenzo[d][1,3]dioxol-5-yl)cyclopropane-1-carboxamido)-3-methylpyridin-2-yl)benzamido)ethyl)-1H-1,2,3-triazol-4-yl)methoxy)ethoxy)propanamido)methyl)phenyl)-2-methyl-7-oxo-2,7-dihydro-6H-pyrazolo[4,3-d]pyrimidin-6-yl)methyl)-4-hydroxypiperidin-1-yl)methyl)benzoate (Compound 16)

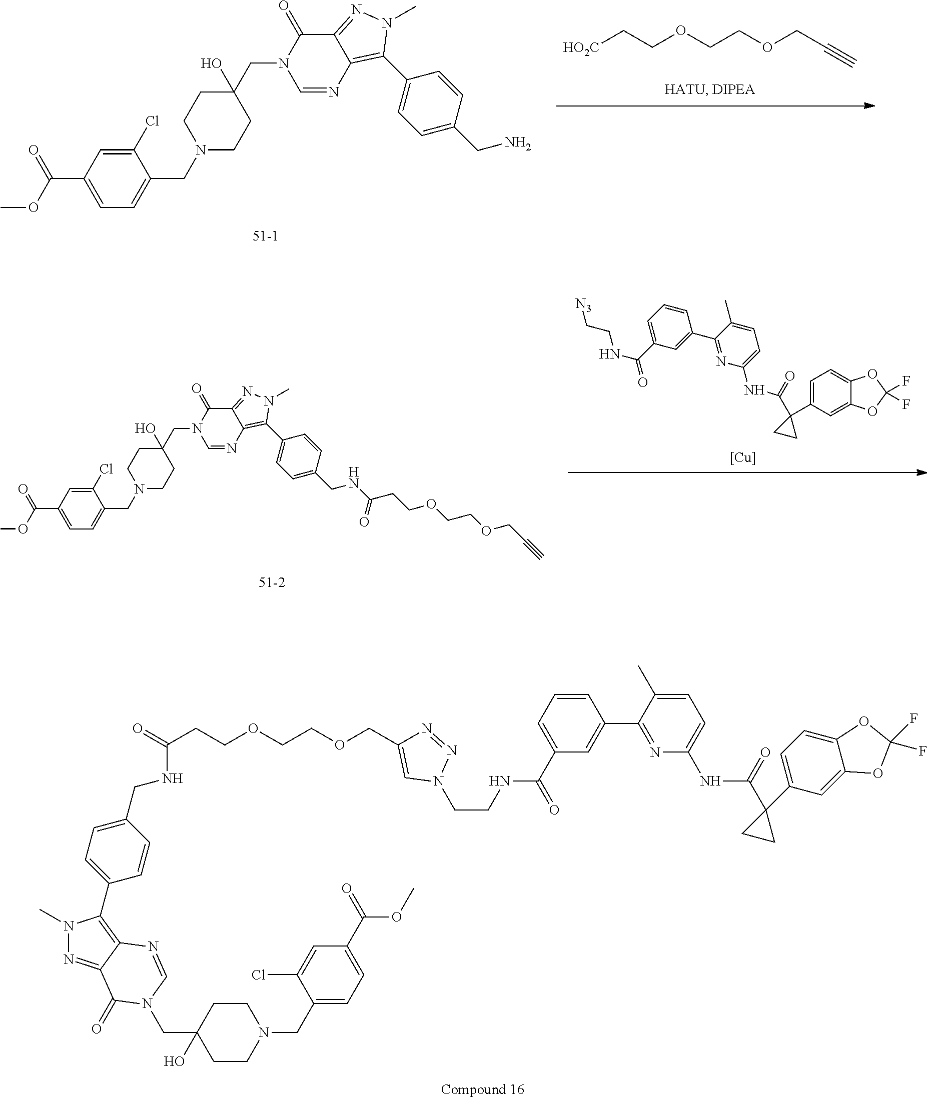

### Example 52. Synthesis of (R)-3-(6-(1-(2,2-difluorobenzo[d][1,3]dioxol-5-yl)cyclopropane-1-carboxamido)-3-methylpyridin-2-yl)-N-(2-((1-((4-hydroxy-1-(3-phenylbutanoyl)piperidin-4-yl)methyl)-6-oxo-1,6-dihydropyrimidin-4-yl)amino)ethyl)benzamide (Compound 17)

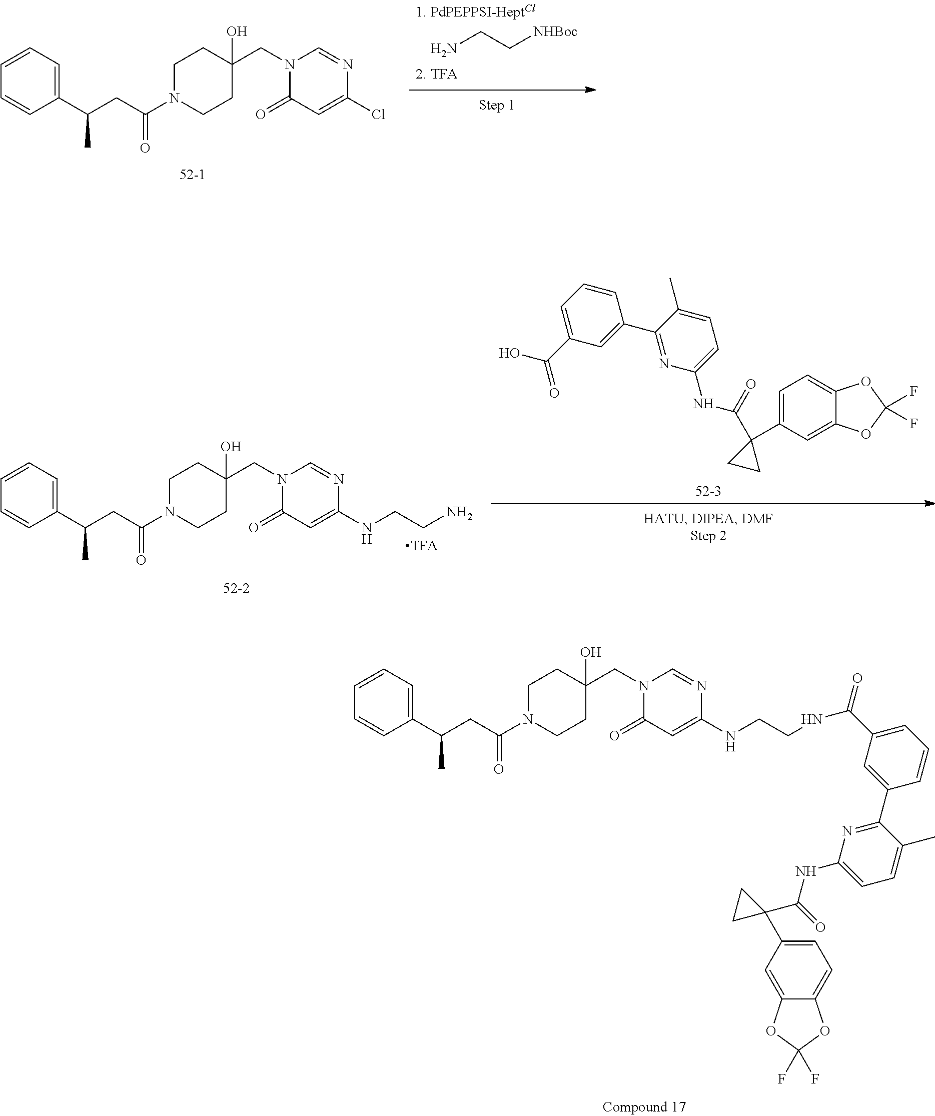

### Example 53. Synthesis of 3-(6-(1-(2,2-difluorobenzo[d][1,3]dioxol-5-yl)cyclopropane-1-carboxamido)-3-methylpyridin-2-yl)-N-(2-(2-(2-((2-((3-((4-hydroxy-1-(3-phenylpropanoyl)piperidin-4-yl)methyl)-4-oxo-3,4-dihydroquinazolin-7-yl)amino)ethyl)amino)-2-oxoethoxy)ethoxy)ethyl)benzamide (Compound 18)

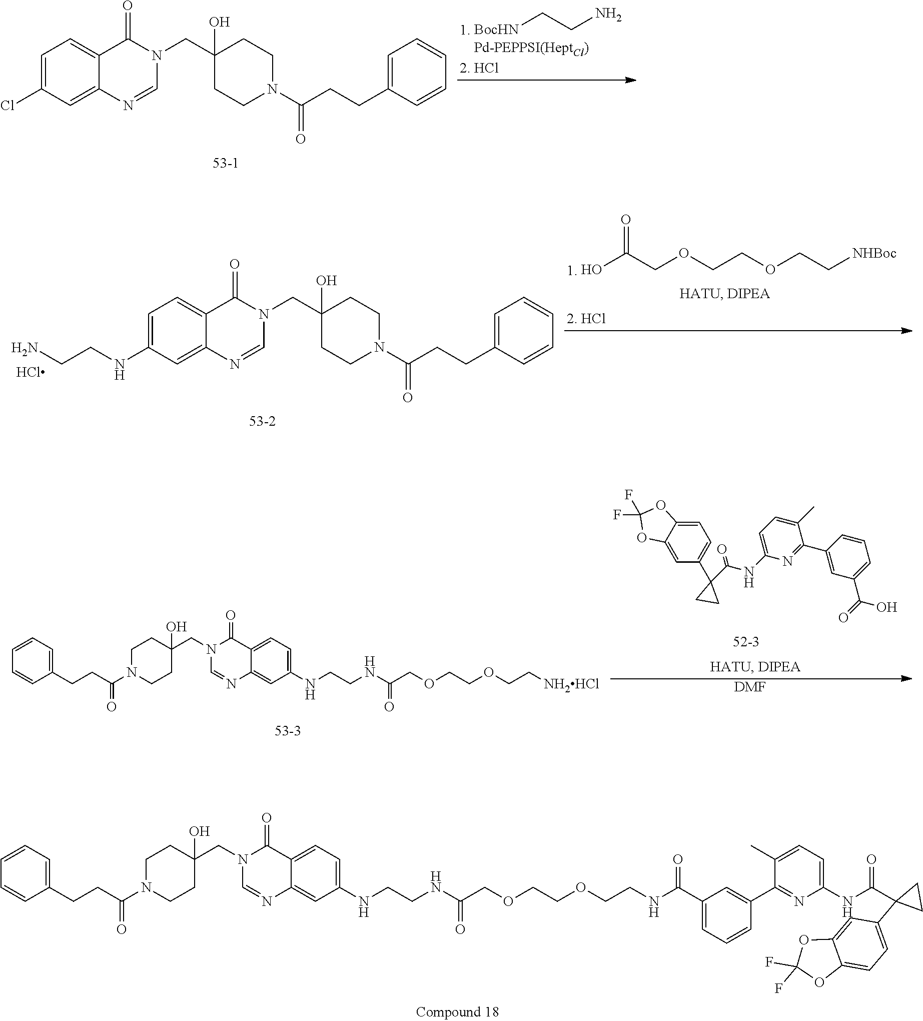

### Example 54. Synthesis of 3-(6-(1-(2,2-difluorobenzo[d][1,3]dioxol-5-yl)cyclopropane-1-carboxamido)-3-methylpyridin-2-yl)-N-(2-(2-(2-(4-(3-(1-methylpiperidin-4-yl)-1,2,4-oxadiazol-5-yl)phenoxy)ethoxy)ethoxy)ethyl)benzamide (Compound 19)

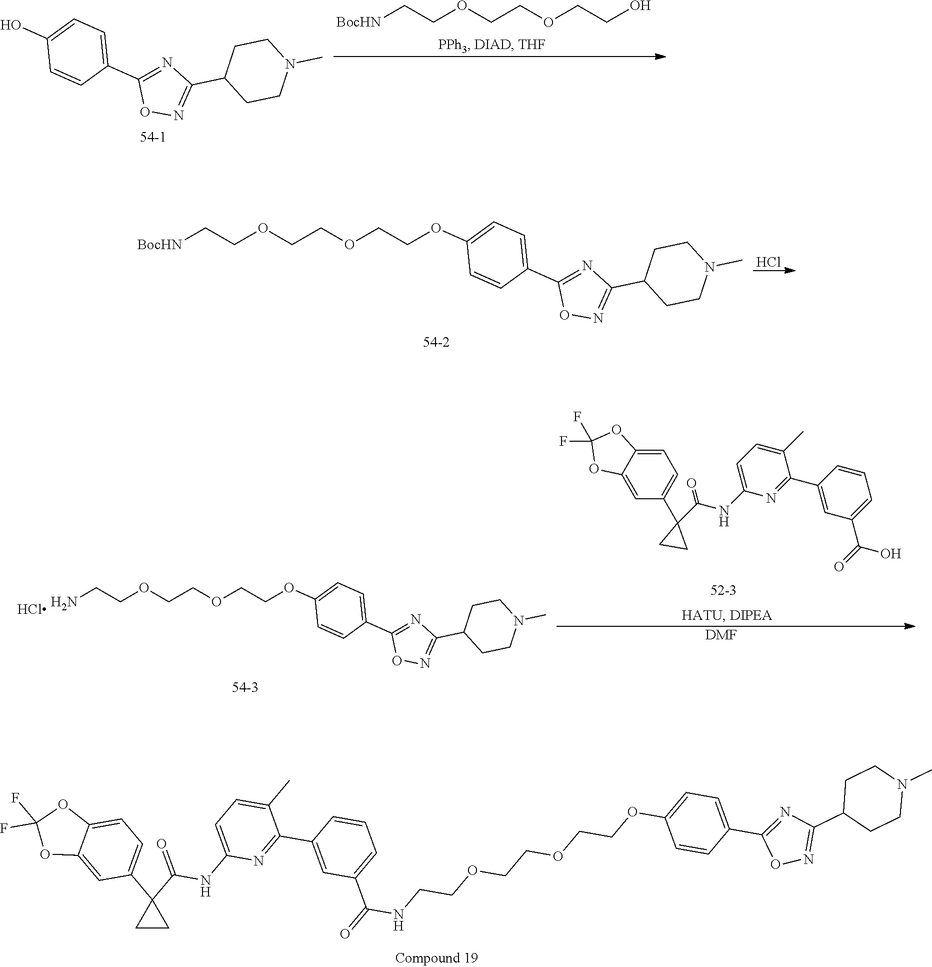

### Example 55. Synthesis of 1-(2,2-difluorobenzo[d][1,3]dioxol-5-yl)-N-(6-(3-((1R,5R)-6-(1-((4-hydroxy-1-((R)-3-phenylbutanoyl)piperidin-4-yl)methyl)-6-oxo-1,6-dihydropyrimidin-4-yl)-3,6-diazabicyclo[3.2.1]octane-3-carbonyl)phenyl)-5-methylpyridin-2-yl)cyclopropane-1-carboxamide (Compound 20)

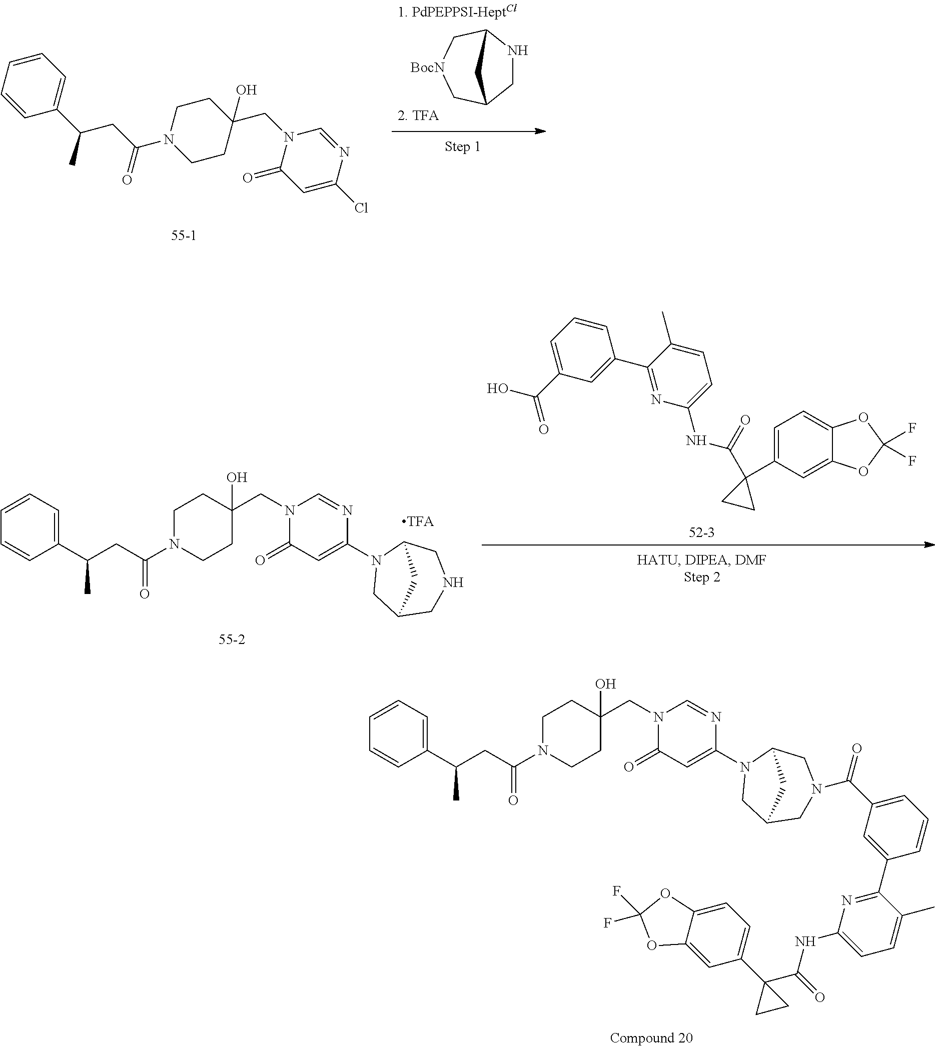

### Example 56. Synthesis of N-(2-(2-(2-(4-(6-amino-5-cyano-3-(1-methyl-1H-pyrrol-3-yl)-2,4-dihydropyrano[2,3-c]pyrazol-4-yl)-2-iodophenoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 21)

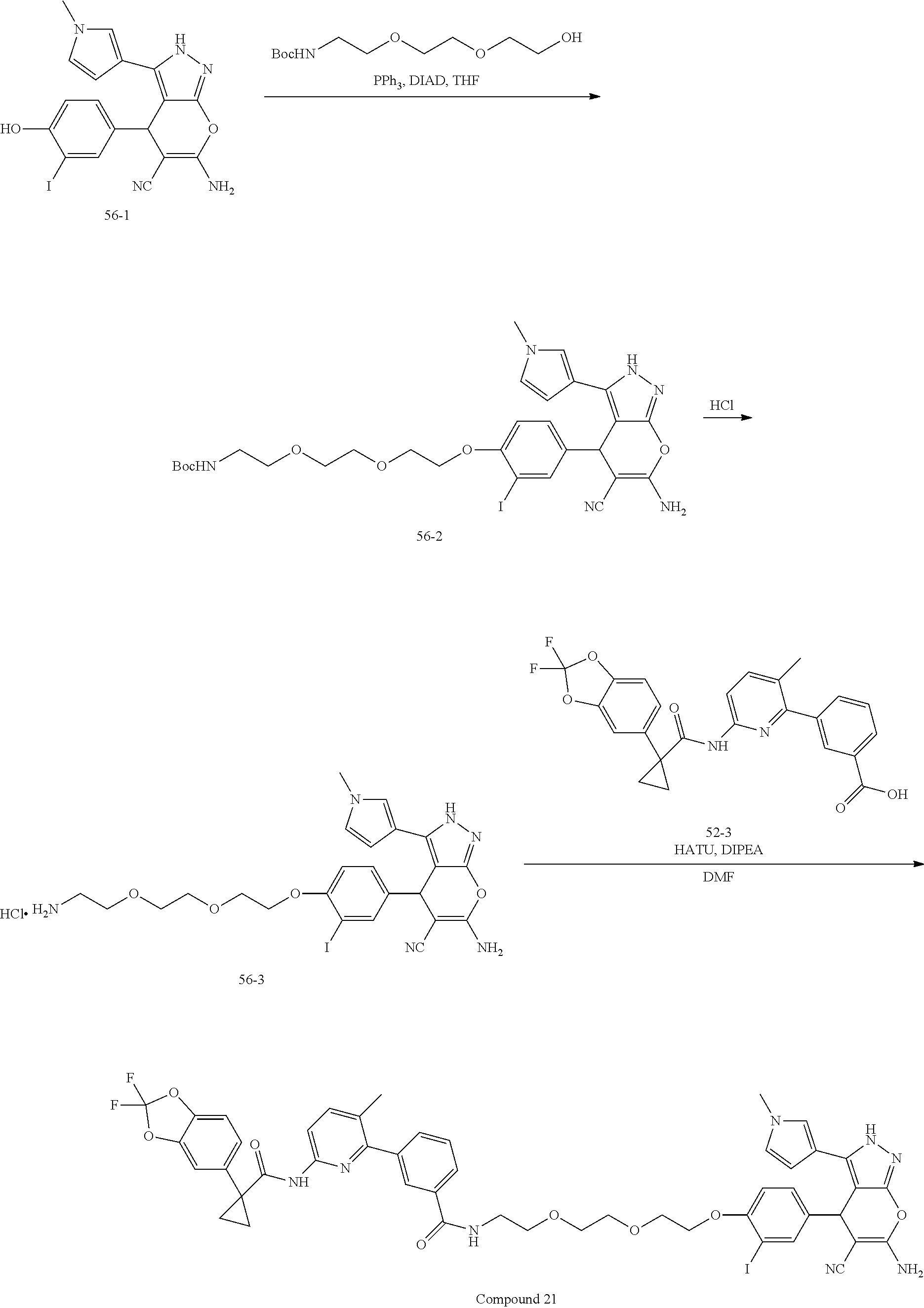

### Example 57 N-(2-(2-(2-(2-aminoethoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Intermediate 57-5)

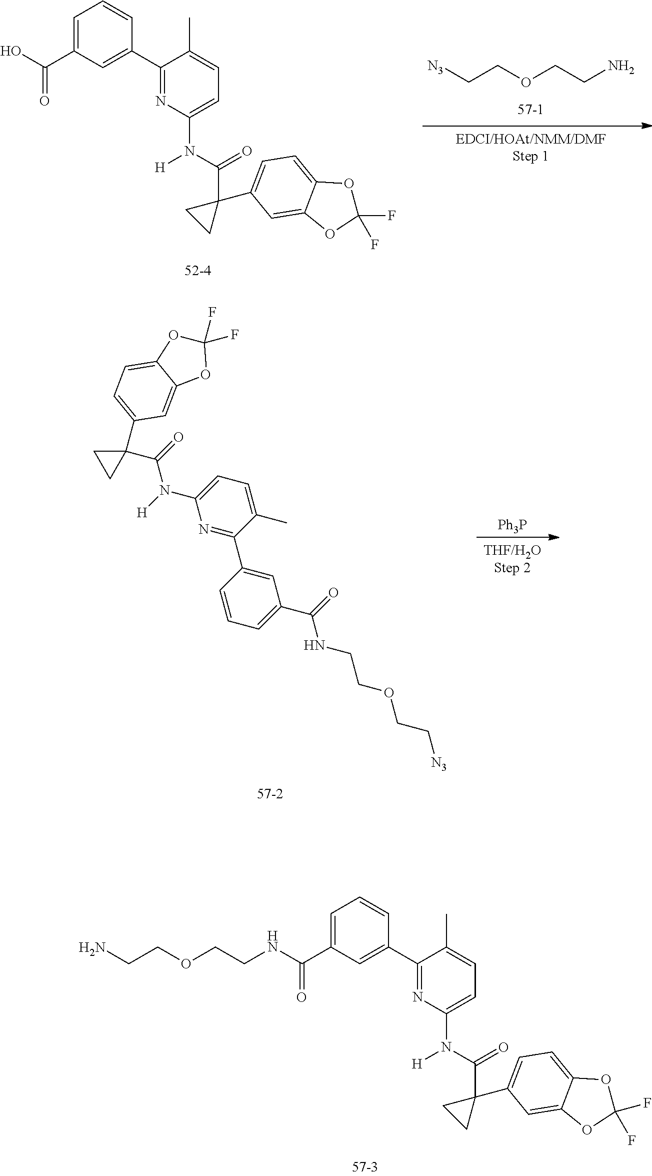 [SMILES: Cc1ccc(NC(=O)C2(c3ccc4c(c3)OC(F)(F)O4)CC2)nc1-c1cccc(C(=O)O)c1 ; [N-]=[N+]=NCCOCCN ; Cc1ccc(NC(=O)C2(c3ccc4c(c3)OC(F)(F)O4)CC2)nc1-c1cccc(C(=O)NCCOCCN=[N+]=[N-])c1 ; "Ph3P" ; "THF/H2OStep 2" ; Cc1ccc(NC(=O)C2(c3ccc4c(c3)OC(F)(F)O4)CC2)nc1-c1cccc(C(=O)NCCOCCN)c1]

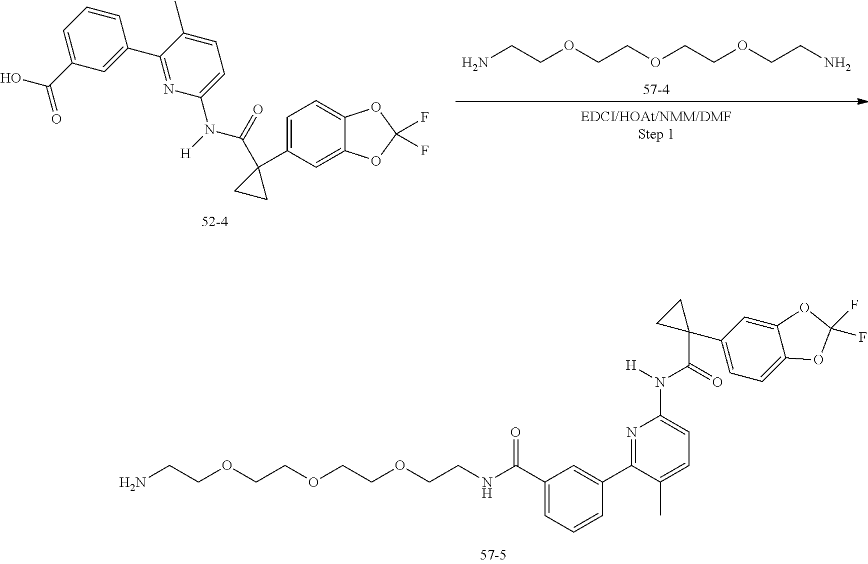

### Example 58. 3-(6-(1-(2,2-difluorobenzo[d][1,3]dioxol-5-yl)cyclopropane-1-carboxamido)-3-methylpyridin-2-yl)-N-(2-(2-(2-(prop-2-yn-1-yloxy)ethoxy)ethoxy)ethyl)benzamide (Intermediate 58-6)

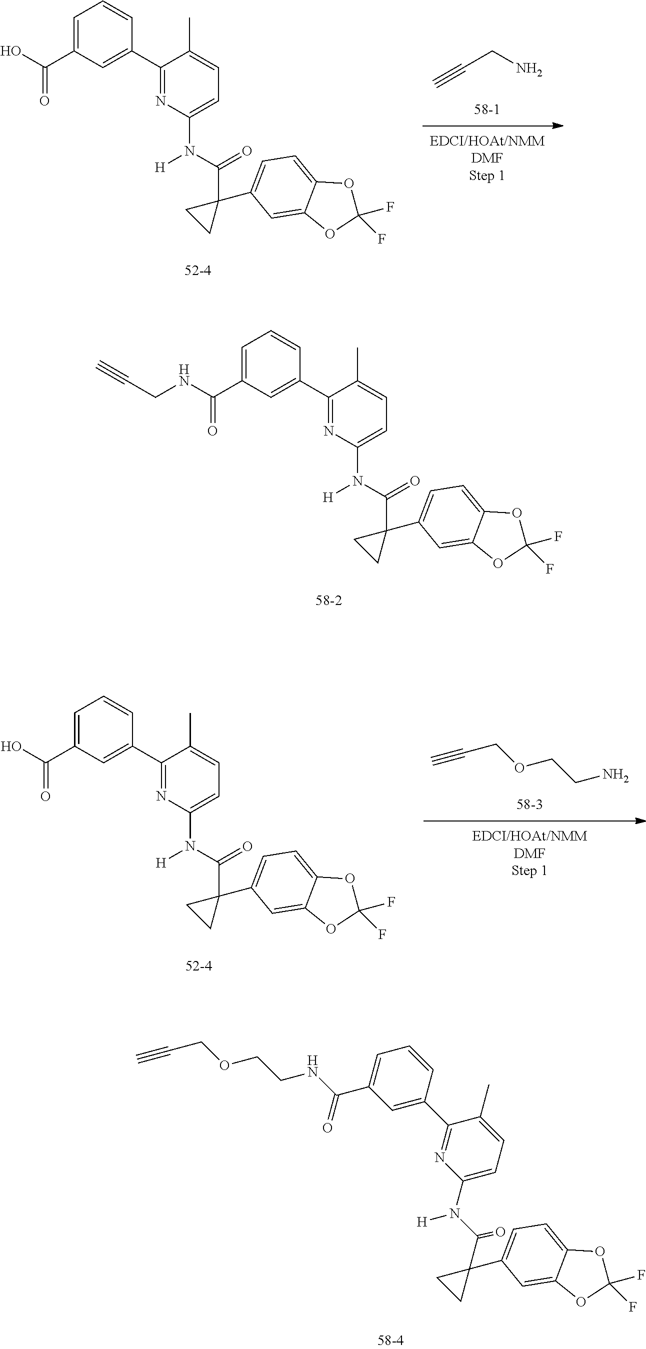

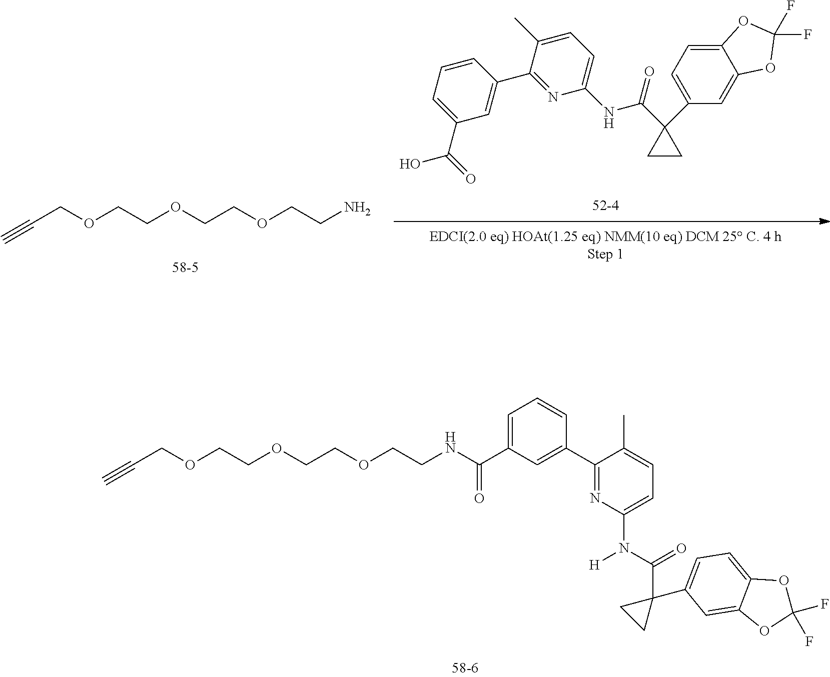 [SMILES: Cc1ccc(NC(=O)C2(c3ccc4c(c3)OC(F)(F)O4)CC2)nc1-c1cccc(C(=O)O)c1 ; C#CCOCCOCCOCCN ; C#CCOCCOCCOCCNC(=O)c1cccc(-c2nc(NC(=O)C3(c4ccc5c(c4)OC(F)(F)O5)CC3)ccc2C)c1]

### Example 59. N-(2-azidoethyl)-3-(6-(1-(2,2-difluorobenzo[d][1,3]dioxol-5-yl)cyclopropane-1-carboxamido)-3-methylpyridin-2-yl)benzamide (Intermediate 59-8)

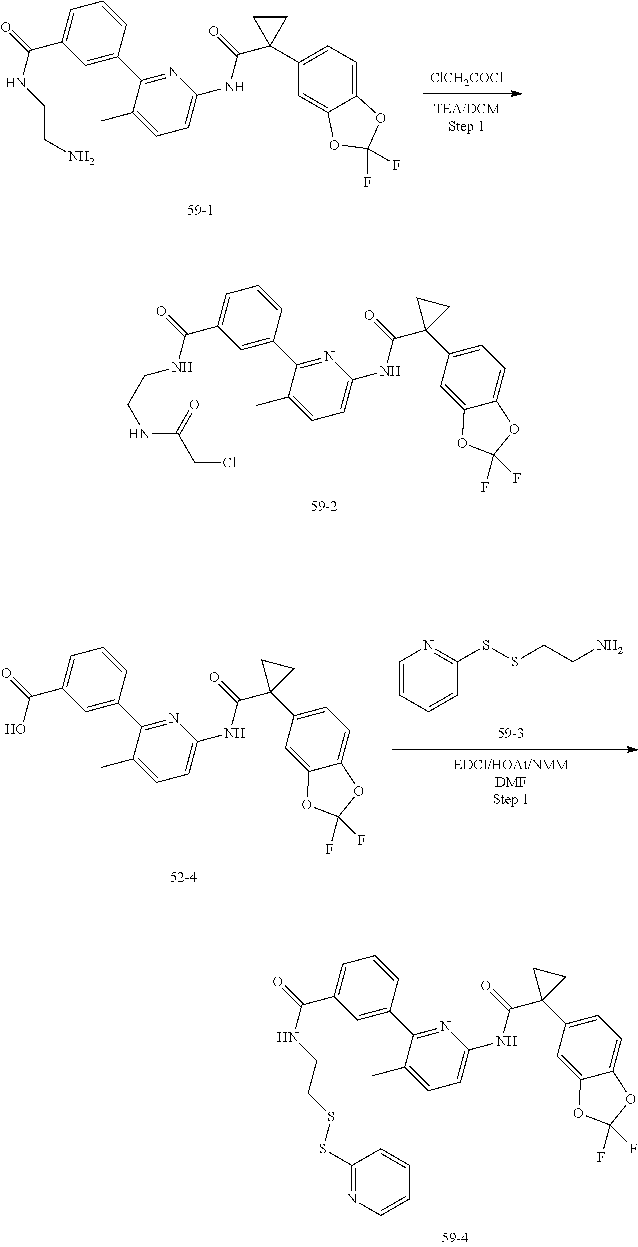 [SMILES: Cc1ccc(NC(=O)C2(c3ccc4c(c3)OC(F)(F)O4)CC2)nc1-c1cccc(C(=O)NCCN)c1 ; "ClCH2COCl" ; Cc1ccc(NC(=O)C2(c3ccc4c(c3)OC(F)(F)O4)CC2)nc1-c1cccc(C(=O)NCCNC(=O)CCl)c1 ; Cc1ccc(NC(=O)C2(c3ccc4c(c3)OC(F)(F)O4)CC2)nc1-c1cccc(C(=O)O)c1 ; NCCSSc1ccccn1 ; Cc1ccc(NC(=O)C2(c3ccc4c(c3)OC(F)(F)O4)CC2)nc1-c1cccc(C(=O)NCCSSc2ccccn2)c1]

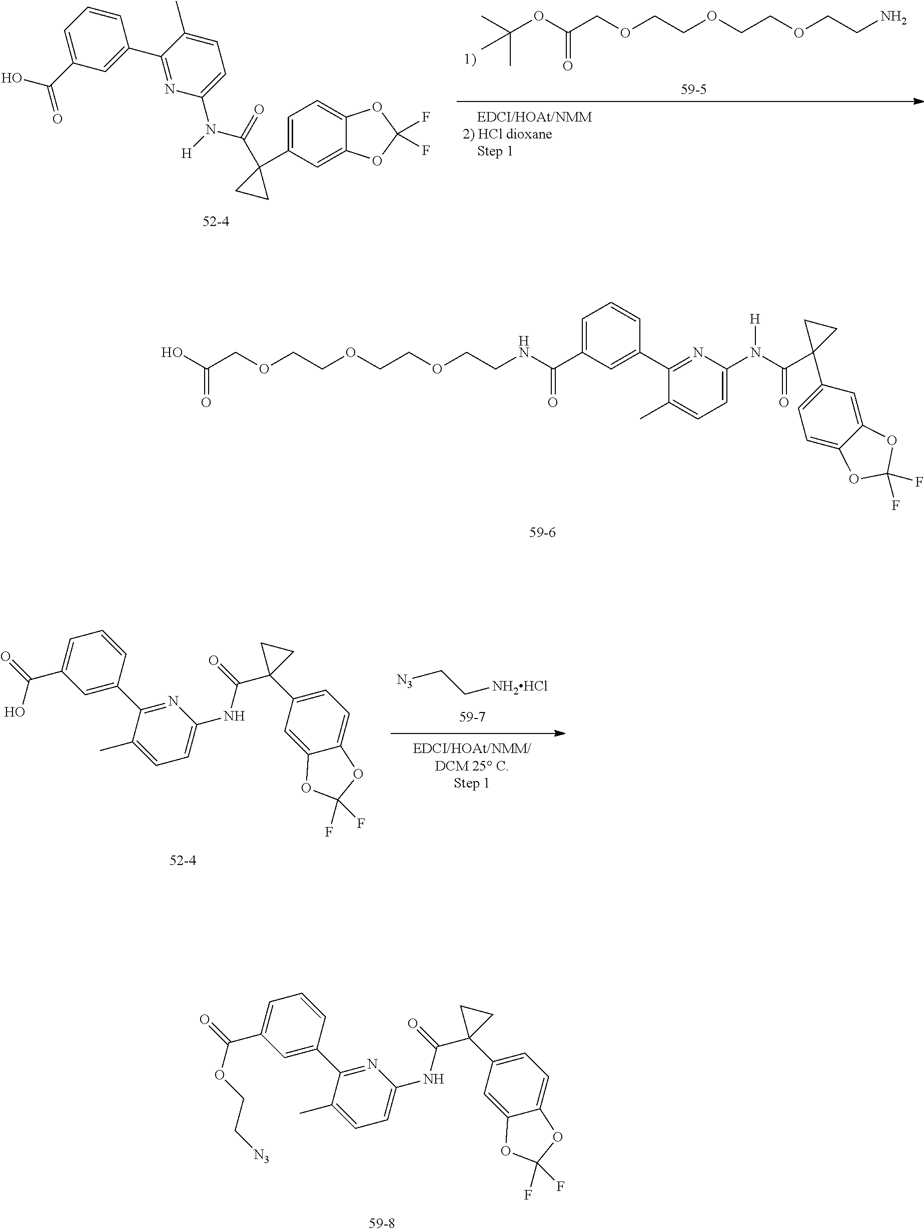

### Example 60. Synthesis of 4,4′-(2-amino-4-ethyl-6-((prop-2-yn-1-yloxy)methyl)pyridine-3,5-diyl)diphenol (Intermediate 60-9)

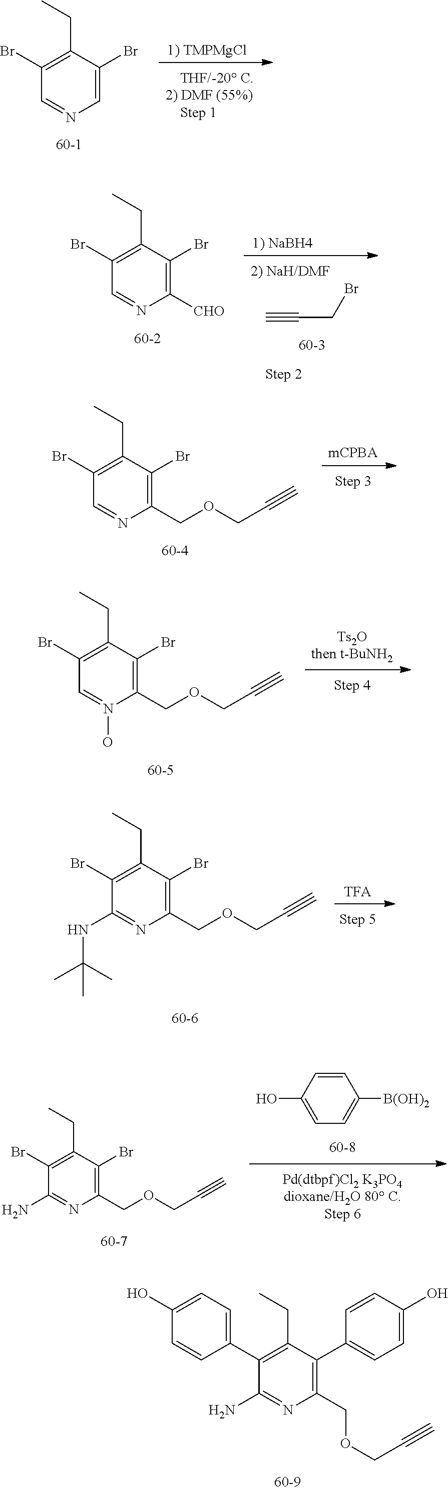

### Example 61. Synthesis of N-(2-(2-(4-(((6-amino-4-ethyl-3,5-bis(4-hydroxyphenyl)pyridin-2-yl)methoxy)methyl)-1H-1,2,3-triazol-1-yl)ethoxy)ethyl)-3-(6-(1-(2,2-difluorobenzo[d][1,3]dioxol-5-yl)cyclopropane-1-carboxamido)-3-methylpyridin-2-yl)benzamide (Compound 22)

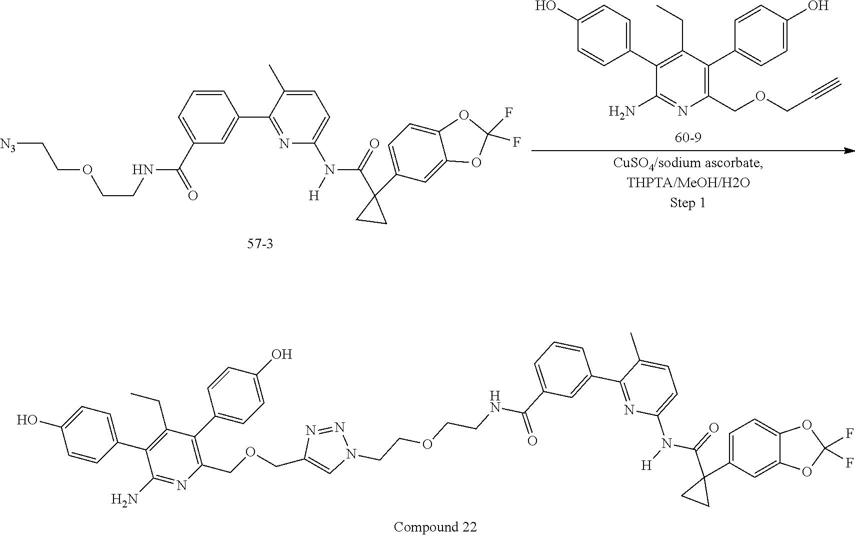 [SMILES: Cc1ccc(NC(=O)C2(c3ccc4c(c3)OC(F)(F)O4)CC2)nc1-c1cccc(C(=O)NCCOCCN=[N+]=[N-])c1 ; C#CCOCc1nc(N)c(-c2ccc(O)cc2)c(CC)c1-c1ccc(O)cc1 ; CCc1c(-c2ccc(O)cc2)c(N)nc(COCc2cn(CCOCCNC(=O)c3cccc(-c4nc(NC(=O)C5(c6ccc7c(c6)OC(F)(F)O7)CC5)ccc4C)c3)nn2)c1-c1ccc(O)cc1]

### Example 62. Synthesis of benzyl (2-(2-aminoethoxy)ethyl)((4-ethyl-3,5-bis(4-hydroxyphenyl)pyridin-2-yl)methyl)carbamate (Intermediate 62-4)

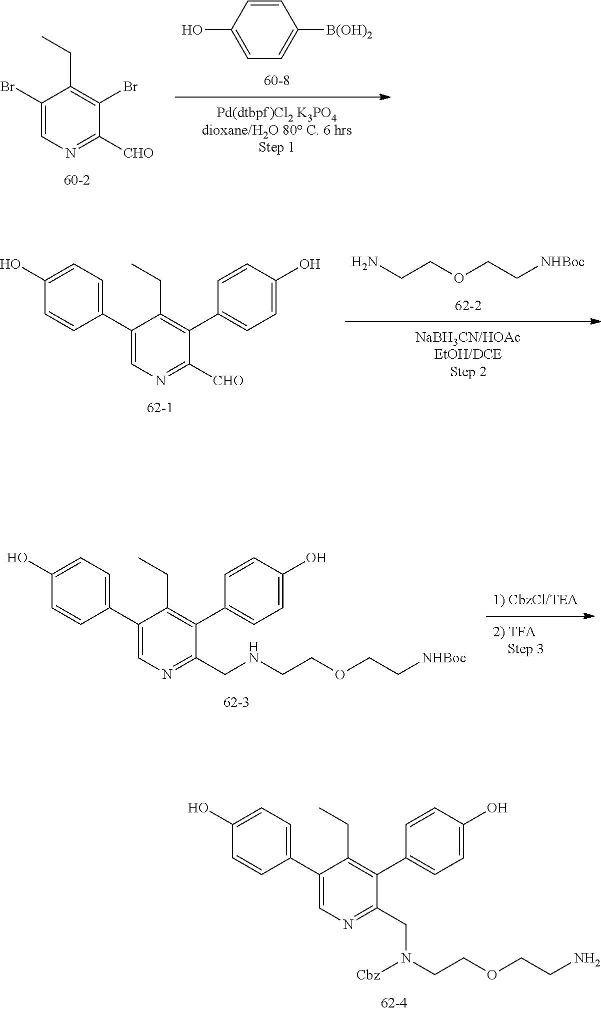 [SMILES: CCc1c(Br)cnc(C=O)c1Br ; OB(O)c1ccc(O)cc1 ; CCc1c(-c2ccc(O)cc2)cnc(C=O)c1-c1ccc(O)cc1 ; CC(C)(C)OC(=O)NCCOCCN ; CCc1c(-c2ccc(O)cc2)cnc(CNCCOCCNC(=O)OC(C)(C)C)c1-c1ccc(O)cc1 ; CCc1c(-c2ccc(O)cc2)cnc(CN(CCOCCN)C(=O)OCc2ccccc2)c1-c1ccc(O)cc1]

### Example 63. Synthesis of 3-(6-(1-(2,2-difluorobenzo[d][1,3]dioxol-5-yl)cyclopropane-1-carboxamido)-3-methylpyridin-2-yl)-N-(2-(2-(((4-ethyl-3,5-bis(4-hydroxyphenyl)pyridin-2-yl)methyl)amino)ethoxy)ethyl)benzamide (Compound 23)

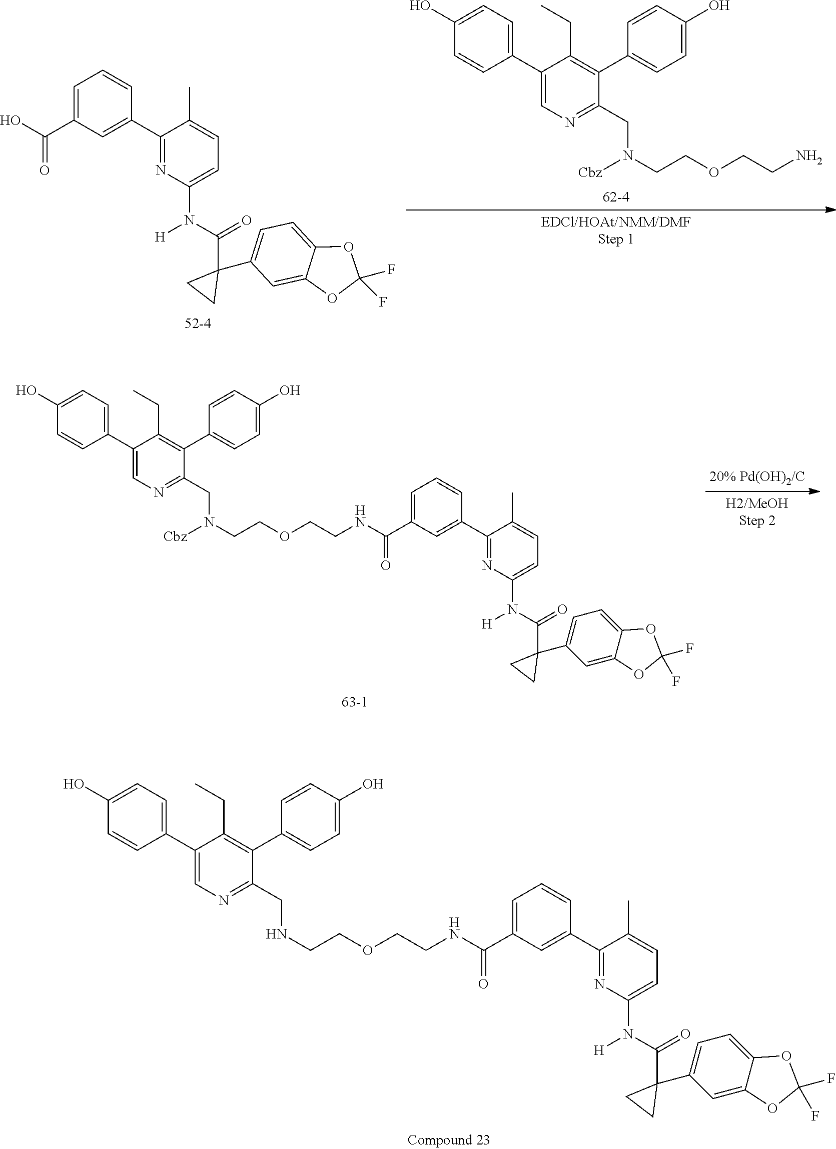 [SMILES: Cc1ccc(NC(=O)C2(c3ccc4c(c3)OC(F)(F)O4)CC2)nc1-c1cccc(C(=O)O)c1 ; CCc1c(-c2ccc(O)cc2)cnc(CN(CCOCCN)C(=O)OCc2ccccc2)c1-c1ccc(O)cc1 ; CCc1c(-c2ccc(O)cc2)cnc(CN(CCOCCNC(=O)c2cccc(-c3nc(NC(=O)C4(c5ccc6c(c5)OC(F)(F)O6)CC4)ccc3C)c2)C(=O)OCc2ccccc2)c1-c1ccc(O)cc1 ; CCc1c(-c2ccc(O)cc2)cnc(CNCCOCCNC(=O)c2cccc(-c3nc(NC(=O)C4(c5ccc6c(c5)OC(F)(F)O6)CC4)ccc3C)c2)c1-c1ccc(O)cc1]

### Example 64. Synthesis of 6′-amino-N-(1-(3-(6-(1-(2,2-difluorobenzo[d][1,3]dioxol-5-yl)cyclopropane-1-carboxamido)-3-methylpyridin-2-yl)phenyl)-1-oxo-5,8,11-trioxa-2-azatridecan-13-yl)-4′-ethyl-5′-(4-hydroxyphenyl)-[3,3′-bipyridine]-6-carboxamide (Compound 24)

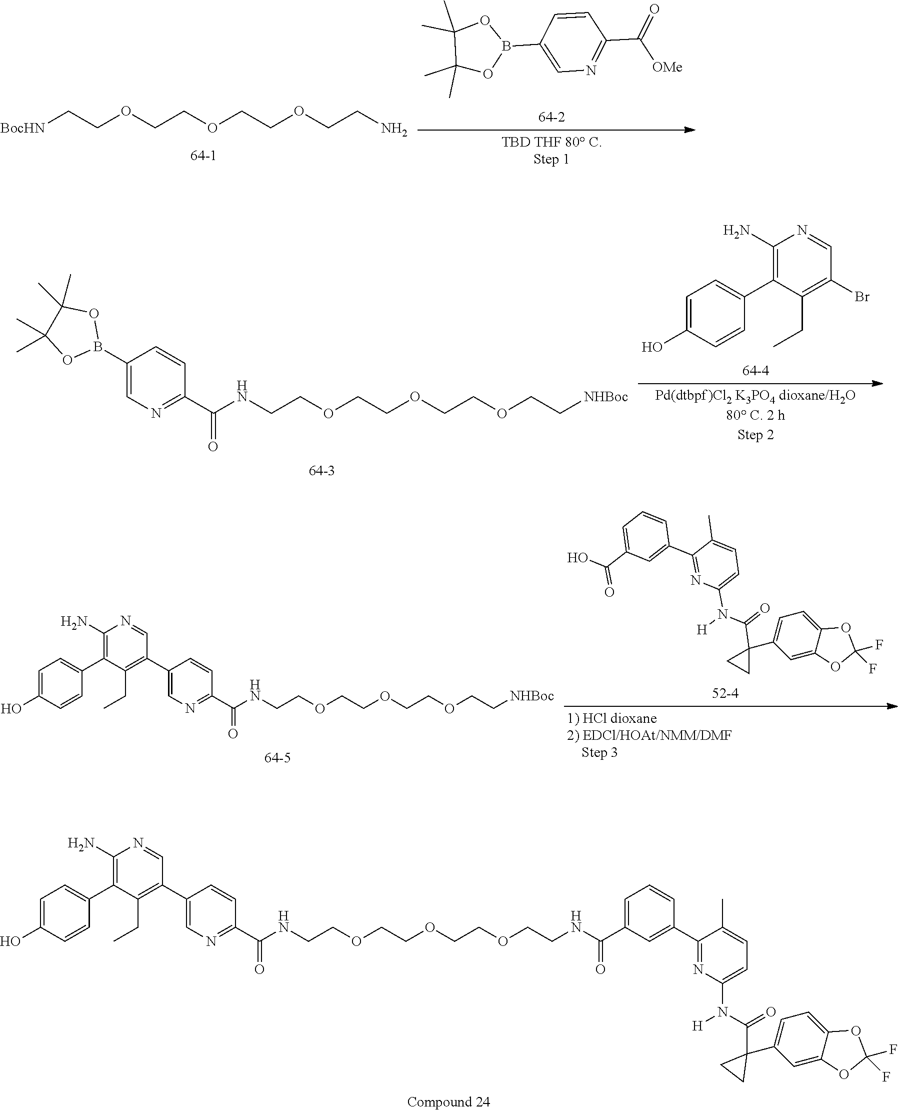

### Example 65. Synthesis of 6′-(aminomethyl)-N-(1-(3-(6-(1-(2,2-difluorobenzo[d][1,3]dioxol-5-yl)cyclopropane-1-carboxamido)-3-methylpyridin-2-yl)phenyl)-1-oxo-5,8,11-trioxa-2-azatridecan-13-yl)-4′-ethyl-5′-(4-hydroxyphenyl)-[3,3′-bipyridine]-6-carboxamide (Compound 25)

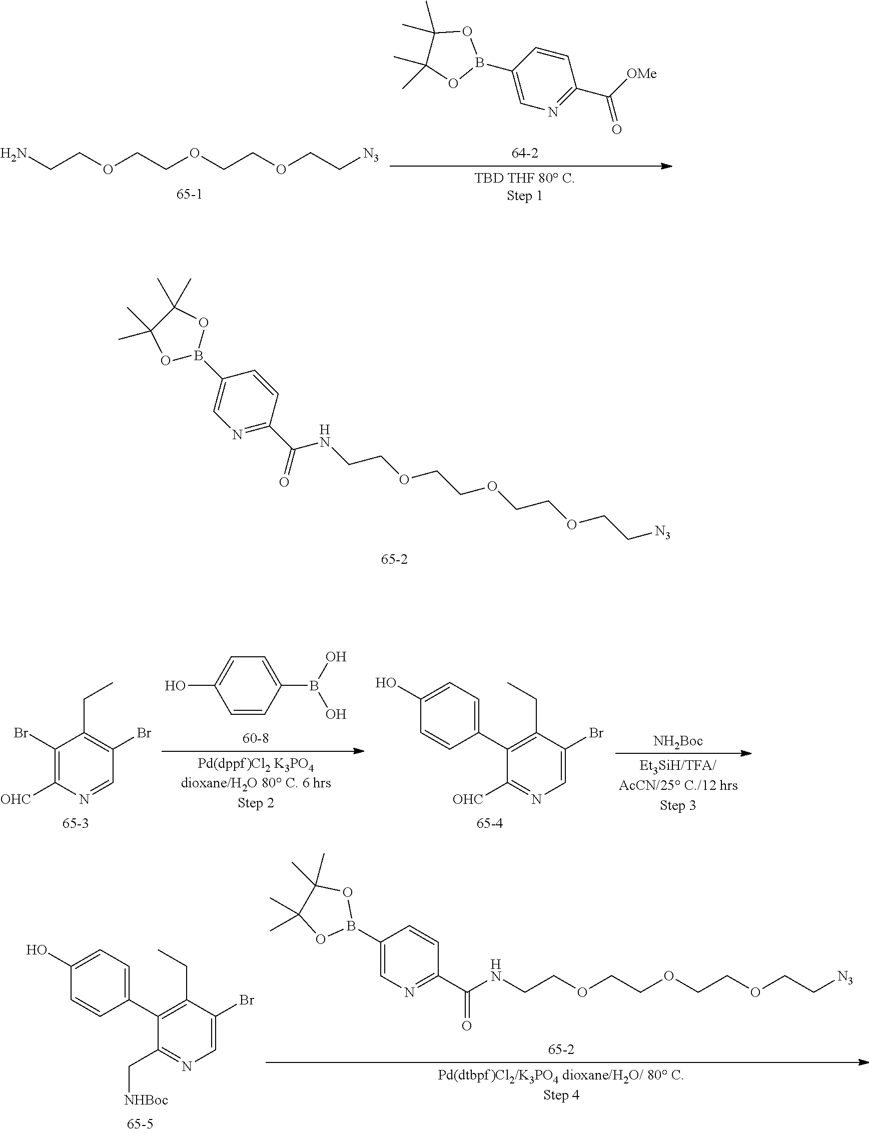

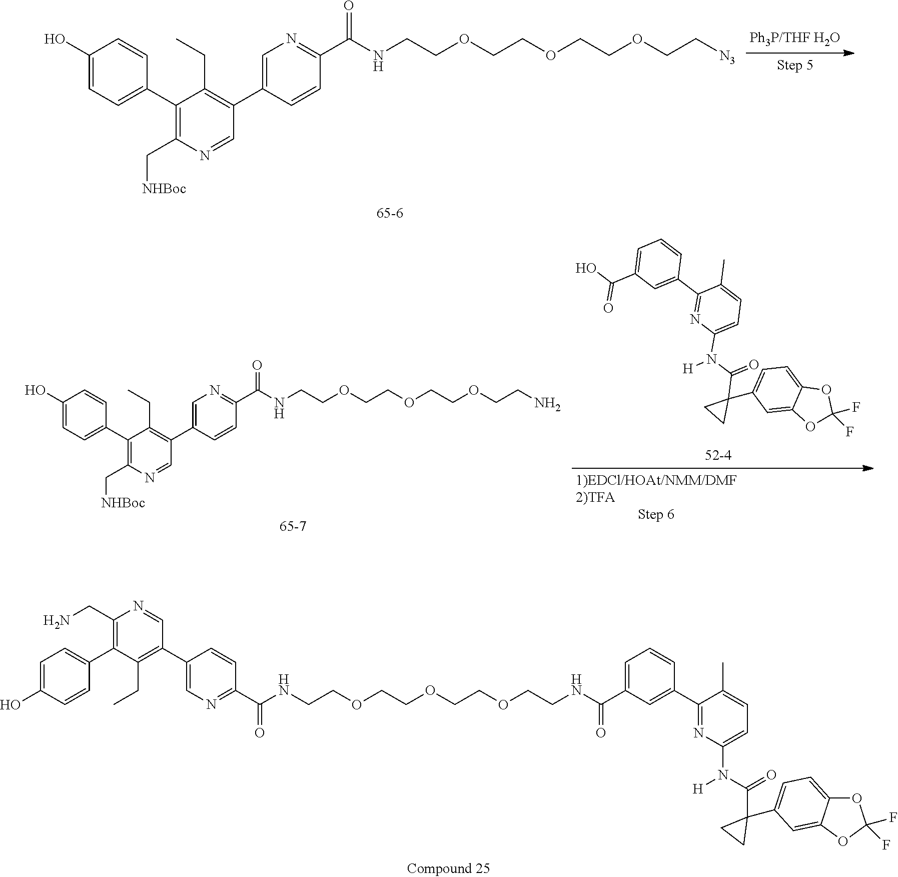 [SMILES: CCc1c(-c2ccc(C(=O)NCCOCCOCCOCCN=[N+]=[N-])nc2)cnc(CNC(=O)OC(C)(C)C)c1-c1ccc(O)cc1 ; CCc1c(-c2ccc(C(=O)NCCOCCOCCOCCN)nc2)cnc(CNC(=O)OC(C)(C)C)c1-c1ccc(O)cc1 ; Cc1ccc(NC(=O)C2(c3ccc4c(c3)OC(F)(F)O4)CC2)nc1-c1cccc(C(=O)O)c1 ; CCc1c(-c2ccc(C(=O)NCCOCCOCCOCCNC(=O)c3cccc(-c4nc(NC(=O)C5(c6ccc7c(c6)OC(F)(F)O7)CC5)ccc4C)c3)nc2)cnc(CN)c1-c1ccc(O)cc1]

### Example 66. Synthesis of 3-((7-(2-(((2S,6S)-4-(2-(2-(2-aminoethoxy)ethoxy)acetyl)-2,6-dimethylpiperazin-1-yl)methyl)-5-chloro-3-methylphenyl)thieno[3,2-b]pyridin-2-yl)methyl)-3-azabicyclo[3.1.0]hexane-2,4-dione (Intermediate 66-7)

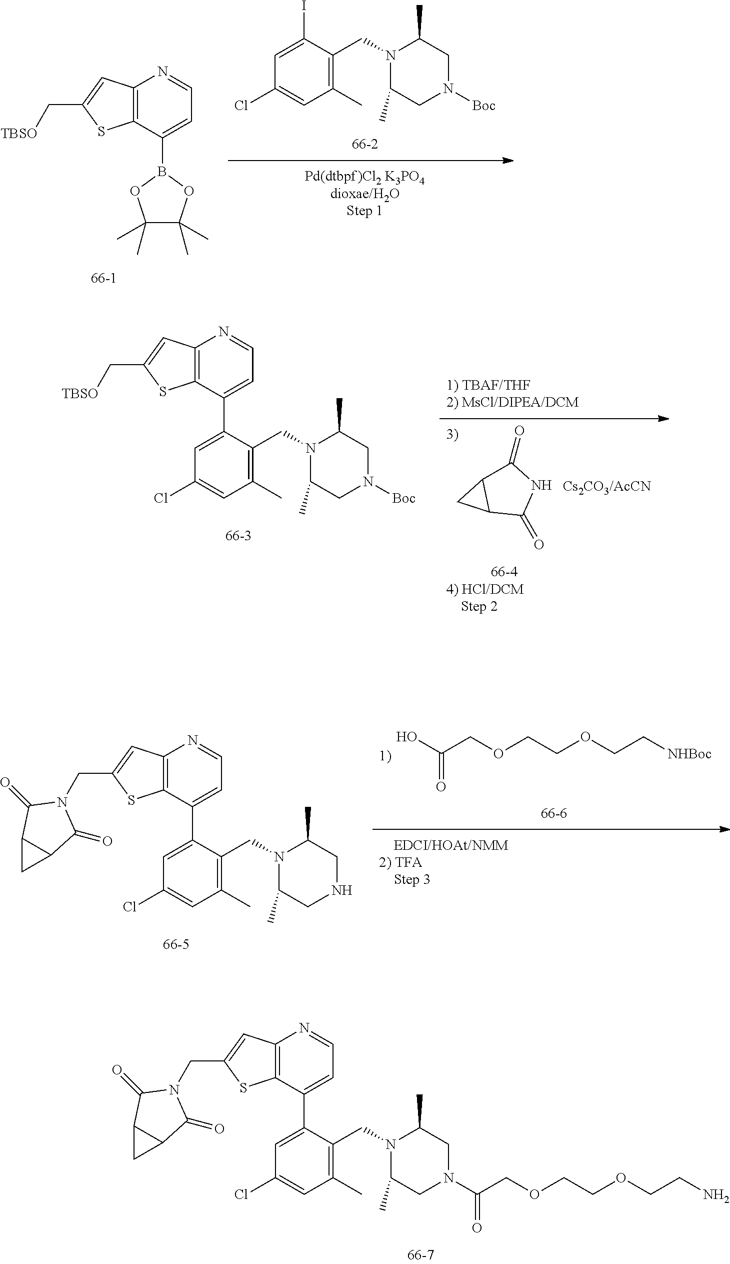

### Example 67. Synthesis of N-(2-(2-(2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-2-oxoethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 26)

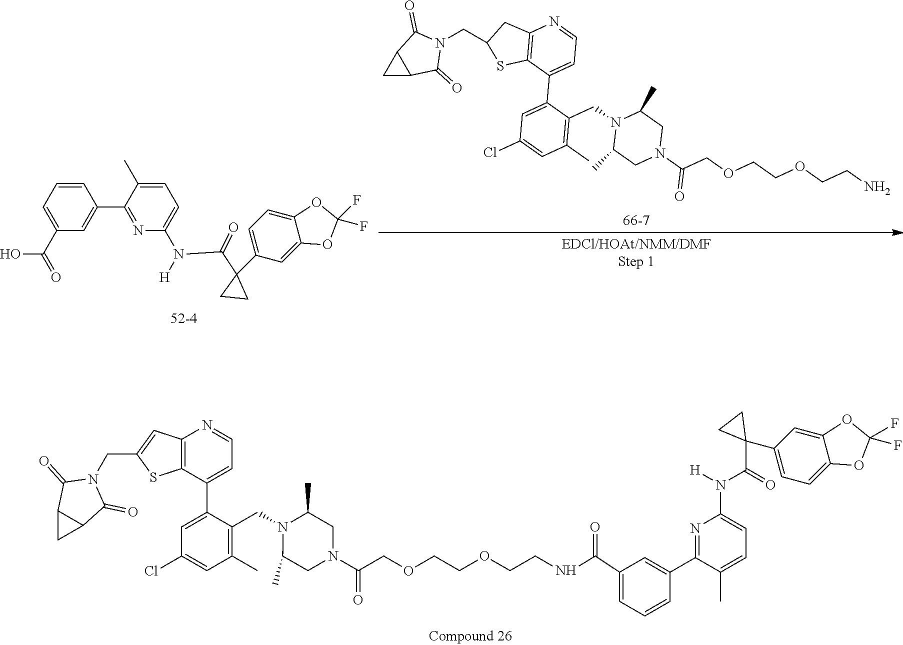

### Example 68. Synthesis of N-(2-(2-((3-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-3-oxopropyl)thio)acetamido)ethyl)-3-(6-(1-(2,2-difluorobenzo[d][1,3]dioxol-5-yl)cyclopropane-1-carboxamido)-3-methylpyridin-2-yl)benzamide (Compound 27)

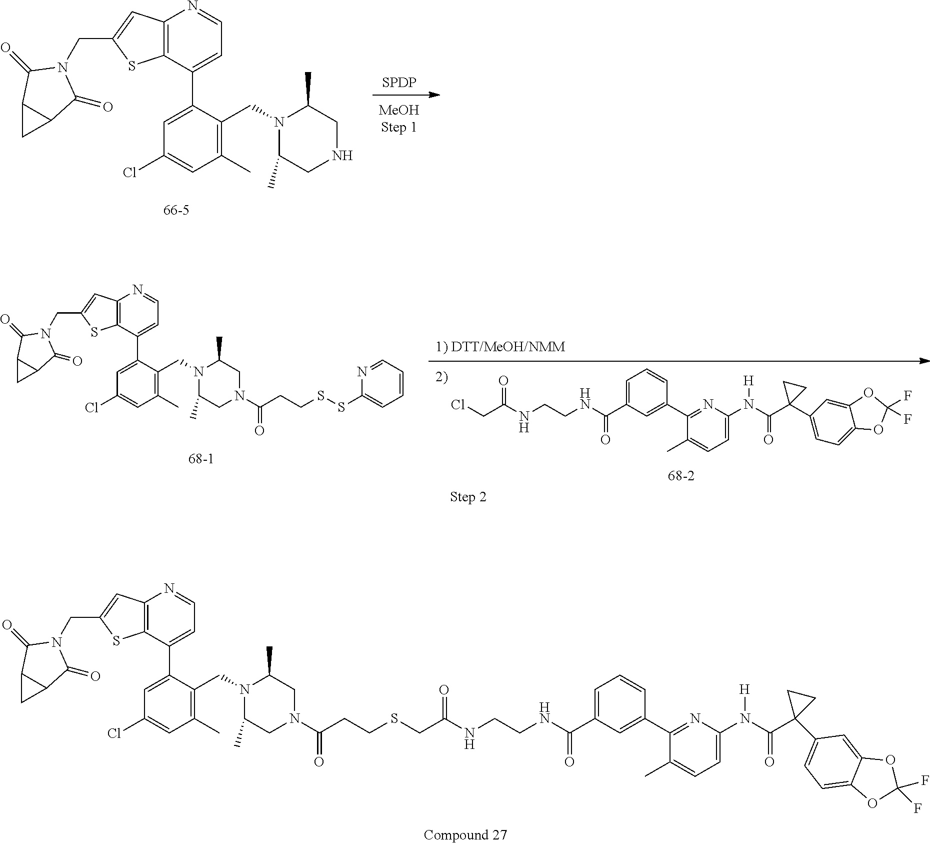

### Example 69. Synthesis of N-(2-(2-((3-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-3-oxopropyl)sulfinyl)acetamido)ethyl)-3-(6-(1-(2,2-difluorobenzo[d][1,3]dioxol-5-yl)cyclopropane-1-carboxamido)-3-methylpyridin-2-yl)benzamide (Compound 28) and N-(2-(2-((3-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-3-oxopropyl)sulfonyl)acetamido)ethyl)-3-(6-(1-(2,2-difluorobenzo[d][1,3]dioxol-5-yl)cyclopropane-1-carboxamido)-3-methylpyridin-2-yl)benzamide (Compound 29)

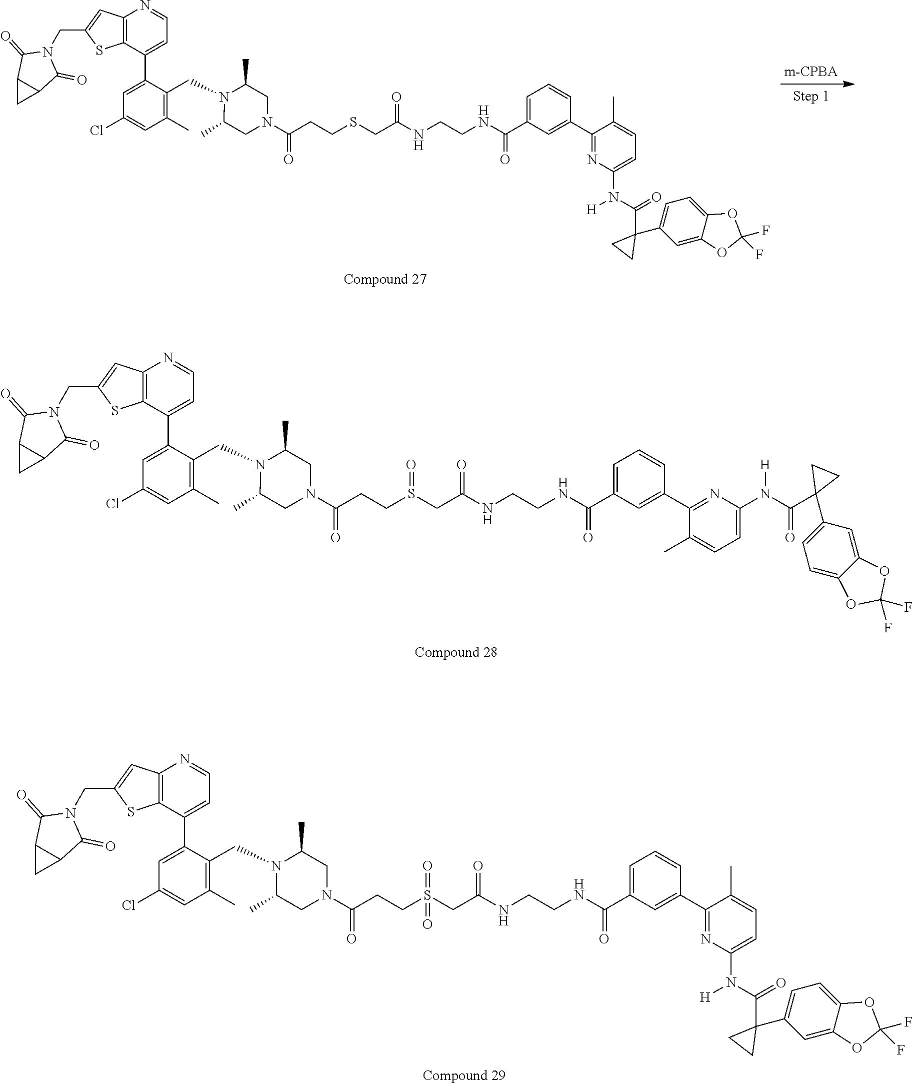

### Example 70. Synthesis of N-(2-((2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-2-oxoethyl)thio)ethyl)-3-(6-(1-(2,2-difluorobenzo[d][1,3]dioxol-5-yl)cyclopropane-1-carboxamido)-3-methylpyridin-2-yl)benzamide (Compound 30)

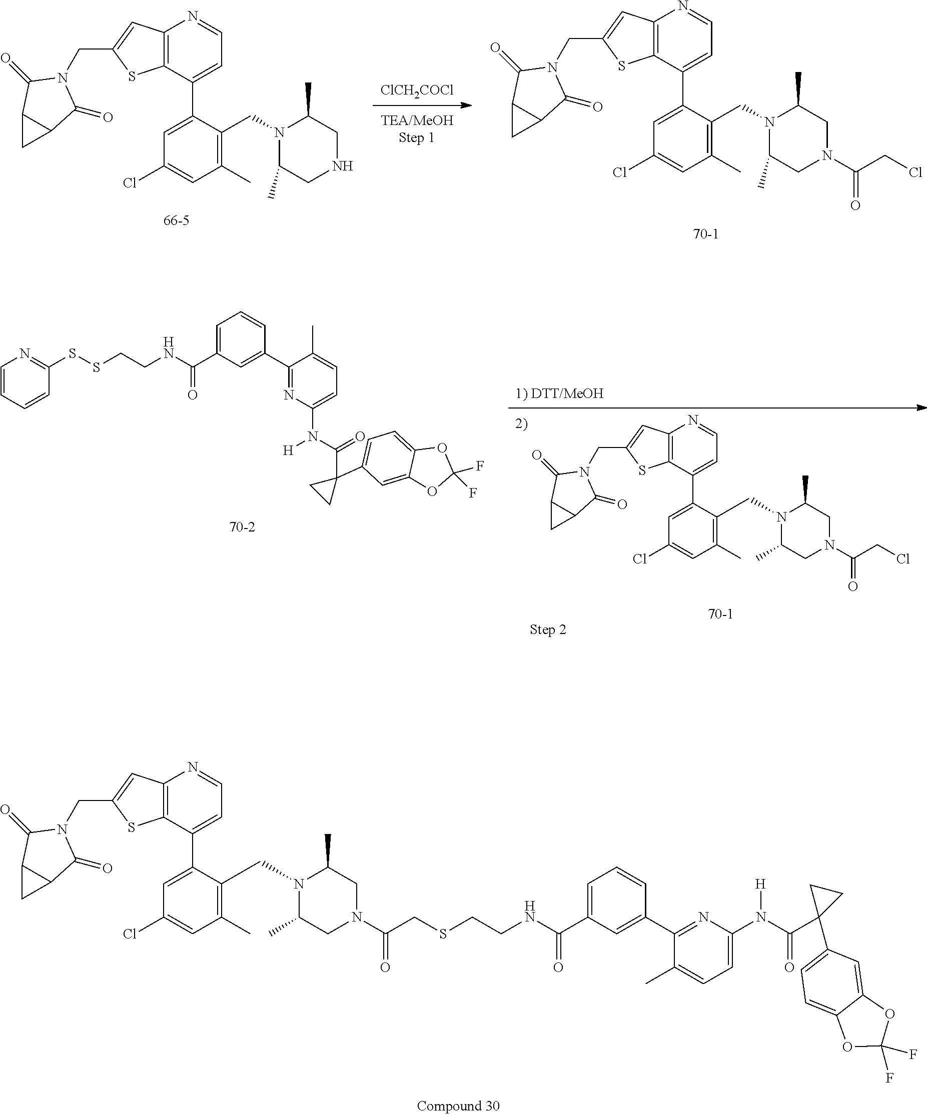

### Example 71. Synthesis of N-(2-((2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-2-oxoethyl)sulfinyl)ethyl)-3-(6-(1-(2,2-difluorobenzo[d][1,3]dioxol-5-yl)cyclopropane-1-carboxamido)-3-methylpyridin-2-yl)benzamide (Compound 31) and N-(2-((2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)-2-oxoethyl)sulfonyl)ethyl)-3-(6-(1-(2,2-difluorobenzo[d][1,3]dioxol-5-yl)cyclopropane-1-carboxamido)-3-methylpyridin-2-yl)benzamide (Compound 32)

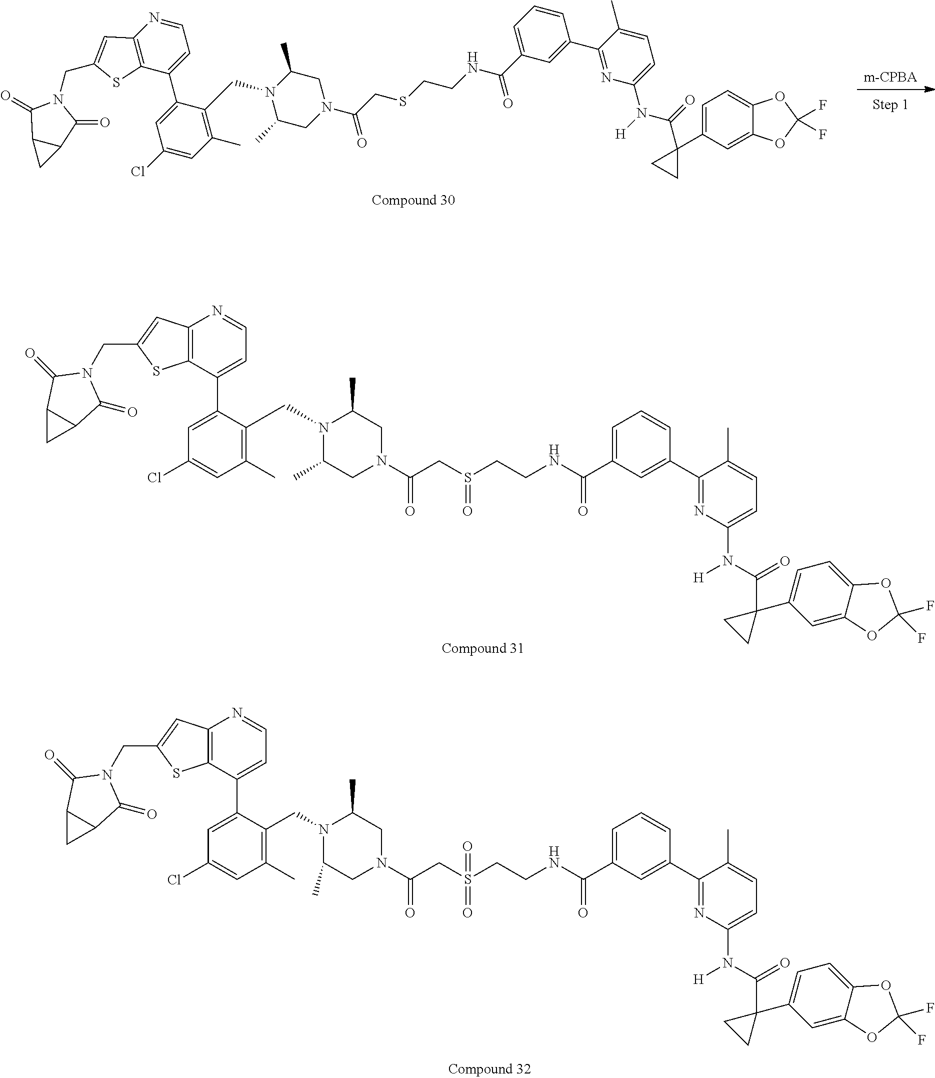

### Example 72. Synthesis of N-(2-((2-(2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)ethoxy)ethyl)thio)ethyl)-3-(6-(1-(2,2-difluorobenzo[d][1,3]dioxol-5-yl)cyclopropane-1-carboxamido)-3-methylpyridin-2-yl)benzamide (Compound 33)

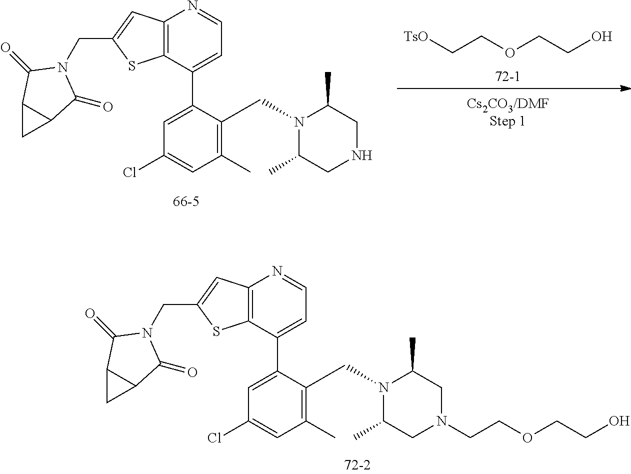

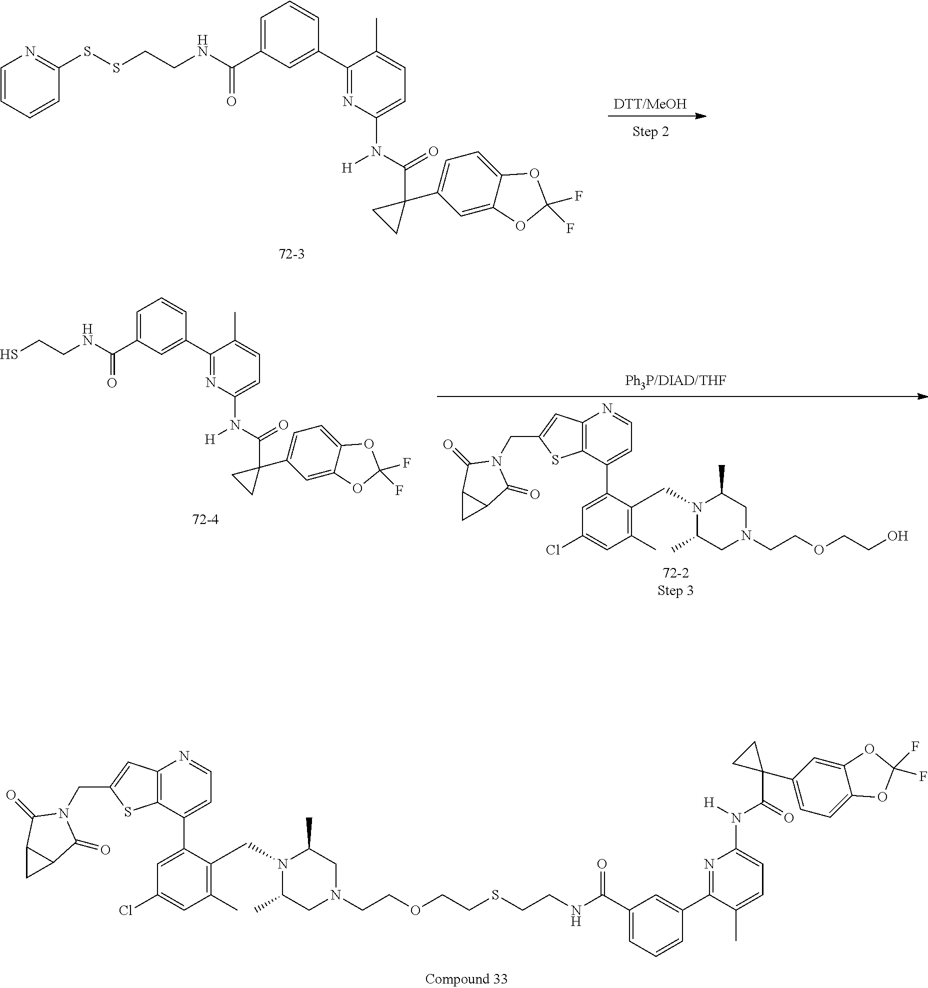

### Example 73. Synthesis of N-(2-((2-(2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)ethoxy)ethyl)sulfinyl)ethyl)-3-(6-(1-(2,2-difluorobenzo[d][1,3]dioxol-5-yl)cyclopropane-1-carboxamido)-3-methylpyridin-2-yl)benzamide (Compound 34) and N-(2-((2-(2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)ethoxy)ethyl)sulfonyl)ethyl)-3-(6-(1-(2,2-difluorobenzo[d][1,3]dioxol-5-yl)cyclopropane-1-carboxamido)-3-methylpyridin-2-yl)benzamide (Compound 35)

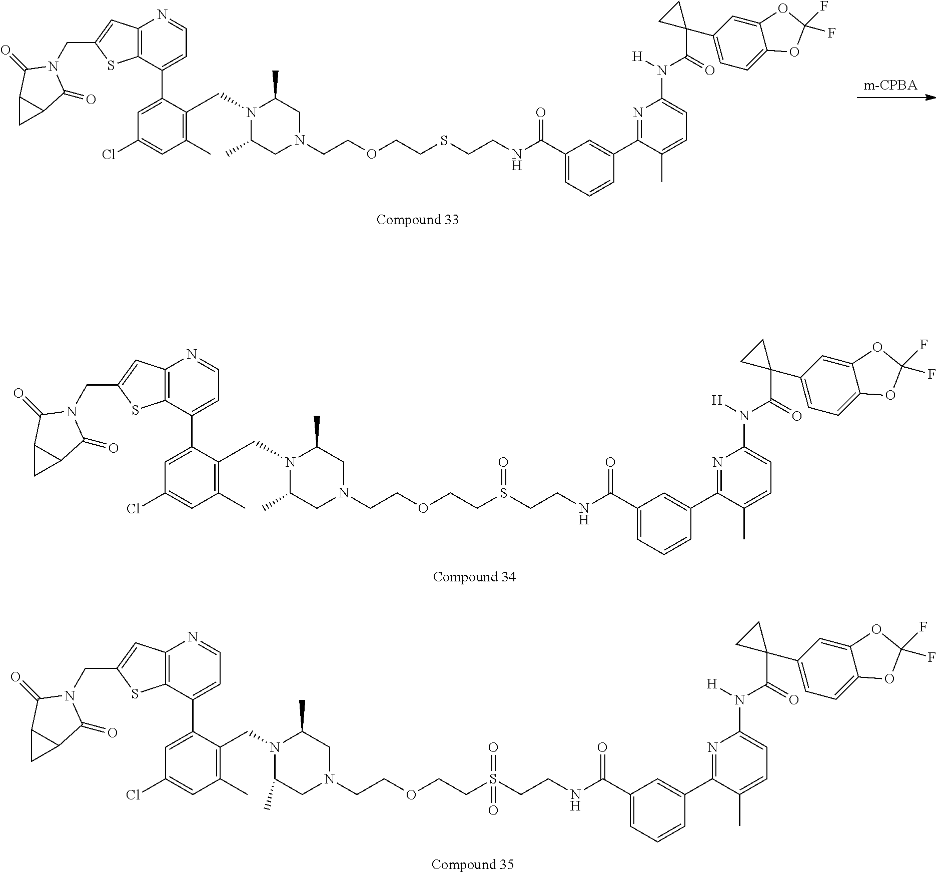

### Example 74. Synthesis of N-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-3-(6-(1-(2,2-difluorobenzo[d][1,3]dioxol-5-yl)cyclopropane-1-carboxamido)-3-methylpyridin-2-yl)benzamide (Compound 36)

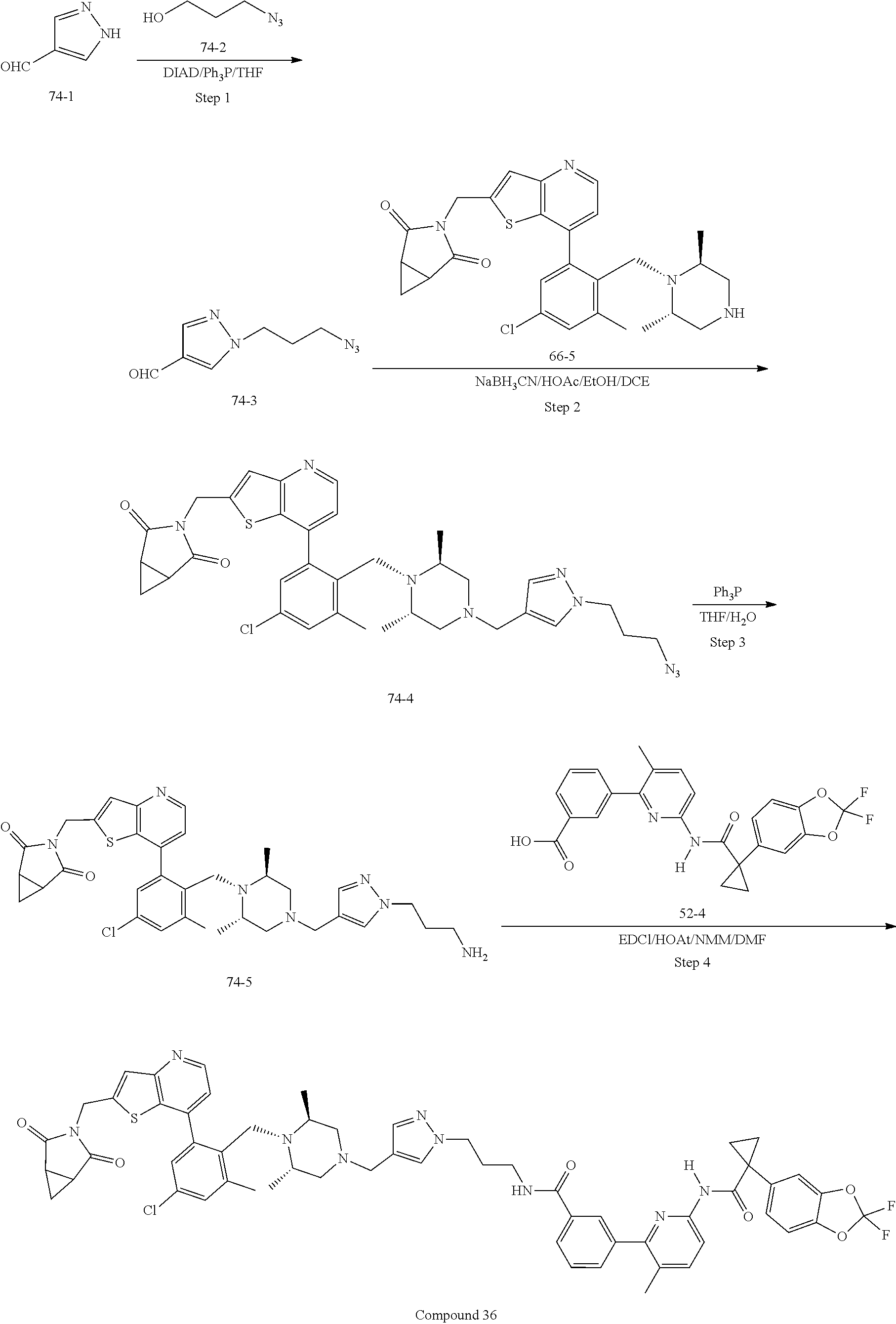

### Example 75. Synthesis of N-((1-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-1H-1,2,3-triazol-4-yl)methyl)-3-(6-(1-(2,2-difluorobenzo[d][1,3]dioxol-5-yl)cyclopropane-1-carboxamido)-3-methylpyridin-2-yl)benzamide (Compound 37)

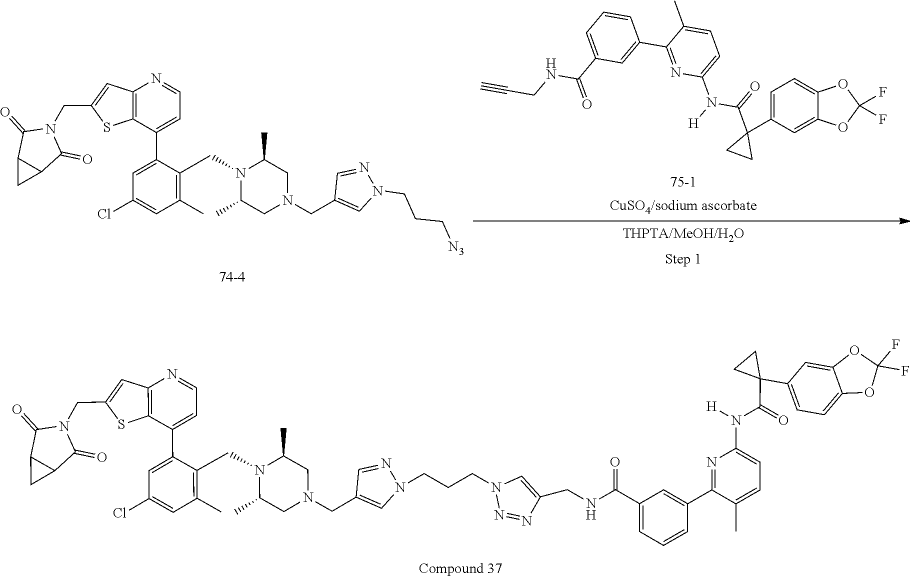

### Example 76. Synthesis of N-(2-(2-(2-(2-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)ethoxy)ethoxy)acetamido)ethyl)-3-(6-(1-(2,2-difluorobenzo[d][1,3]dioxol-5-yl)cyclopropane-1-carboxamido)-3-methylpyridin-2-yl)benzamide (Compound 38)

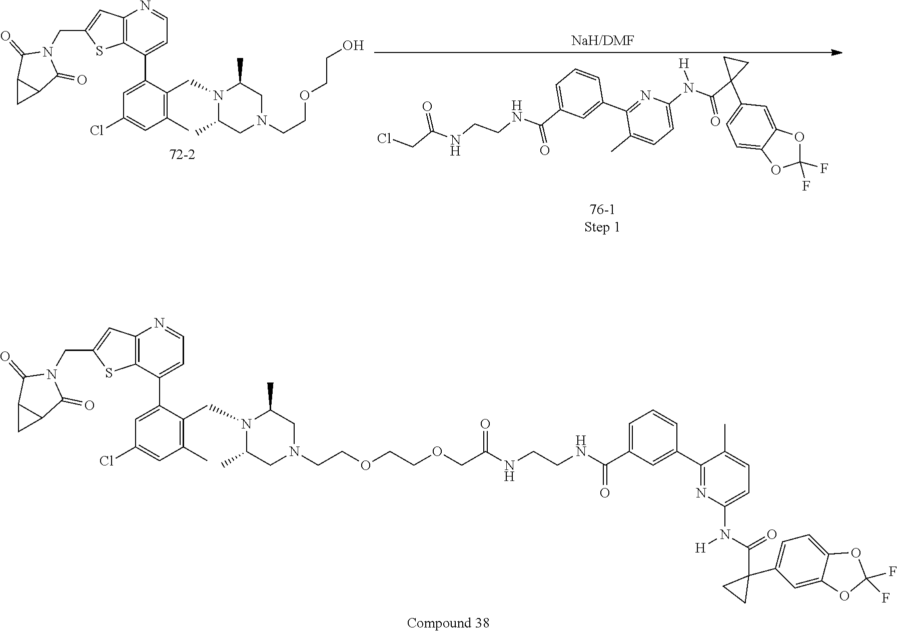

### Example 77. Synthesis of N-(3-((3-(4-(4-chloro-2-(2-((2,5-dioxopyrrolidin-1-yl)methyl)thieno[3,2-b]pyridin-7-yl)-6-methylphenoxy)piperidin-1-yl)propyl)amino)-3-oxopropyl)-3-(6-(1-(2,2-difluorobenzo[d][1,3]dioxol-5-yl)cyclopropane-1-carboxamido)-3-methylpyridin-2-yl)benzamide (Compound 39)

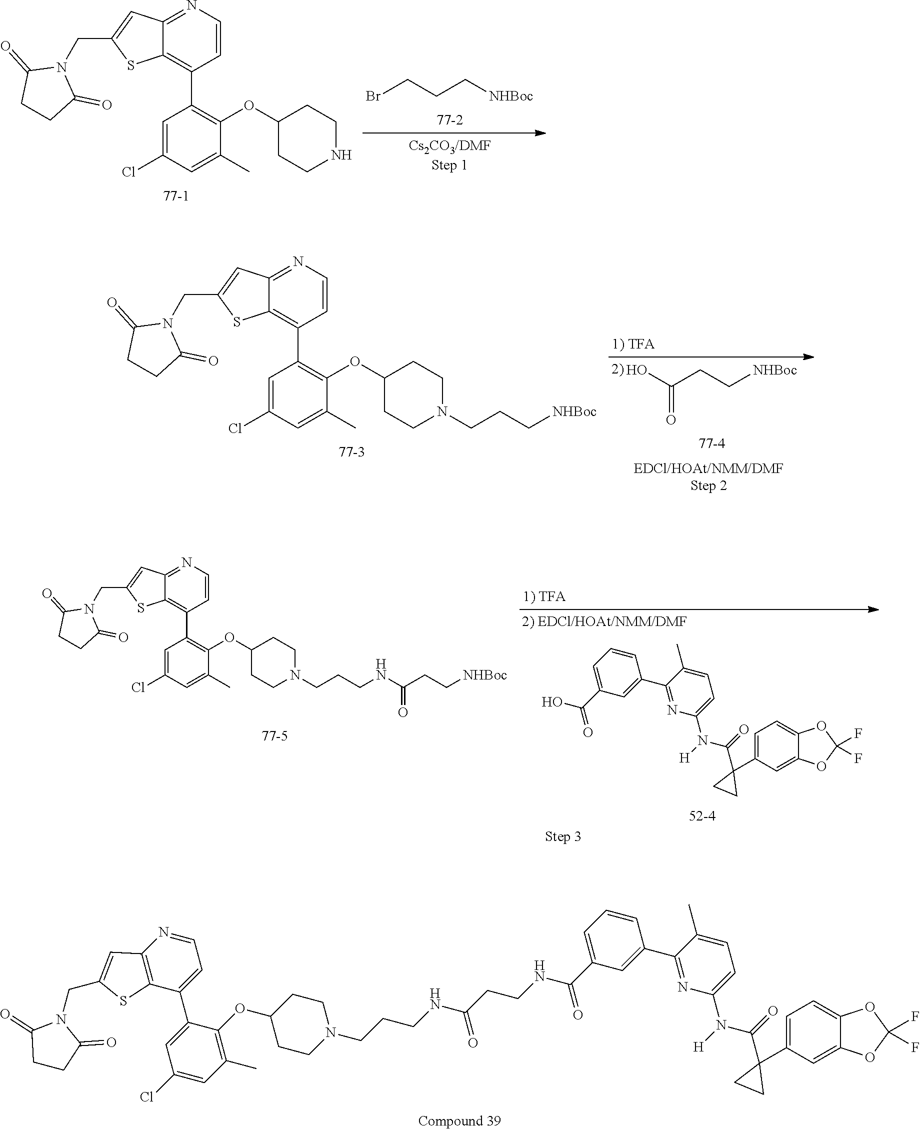

### Example 78. Synthesis of N-(2-(2-(3-(4-(4-chloro-2-(2-((2,5-dioxopyrrolidin-1-yl)methyl)thieno[3,2-b]pyridin-7-yl)-6-methylphenoxy)piperidin-1-yl)-3-oxopropoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 40)

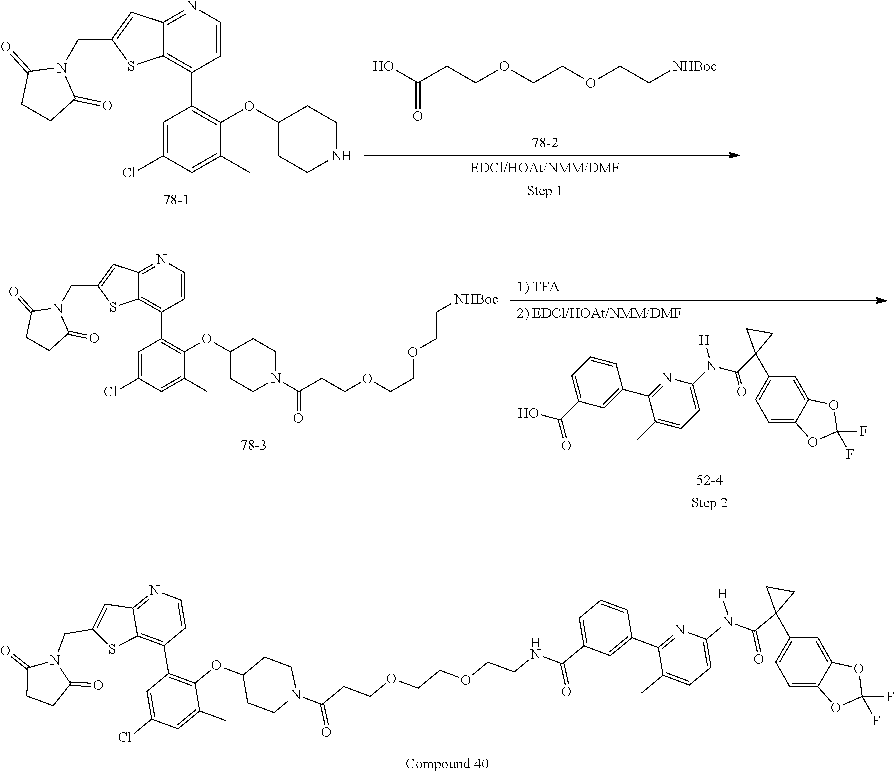 [SMILES: Cc1cc(Cl)cc(-c2ccnc3cc(CN4C(=O)CCC4=O)sc23)c1OC1CCNCC1 ; CC(C)(C)OC(=O)NCCOCCOCCC(=O)O ; Cc1cc(Cl)cc(-c2ccnc3cc(CN4C(=O)CCC4=O)sc23)c1OC1CCN(C(=O)CCOCCOCCNC(=O)OC(C)(C)C)CC1 ; Cc1ccc(NC(=O)C2(c3ccc4c(c3)OC(F)(F)O4)CC2)nc1-c1cccc(C(=O)O)c1 ; Cc1ccc(NC(=O)C2(c3ccc4c(c3)OC(F)(F)O4)CC2)nc1-c1cccc(C(=O)NCCOCCOCCC(=O)N2CCC(Oc3c(C)cc(Cl)cc3-c3ccnc4cc(CN5C(=O)CCC5=O)sc34)CC2)c1]

### Example 79. Synthesis of (R)—N-((1-(2-(2-(2-(4-(7-(2-aminopyridin-4-yl)-5-chloro-2,3-dihydrobenzofuran-2-carbonyl)piperazin-1-yl)ethoxy)ethoxy)ethyl)-1H-1,2,3-triazol-4-yl)methyl)-3-(6-(1-(2,2-difluorobenzo[d][1,3]dioxol-5-yl)cyclopropane-1-carboxamido)-3-methylpyridin-2-yl)benzamide (Compound 41)

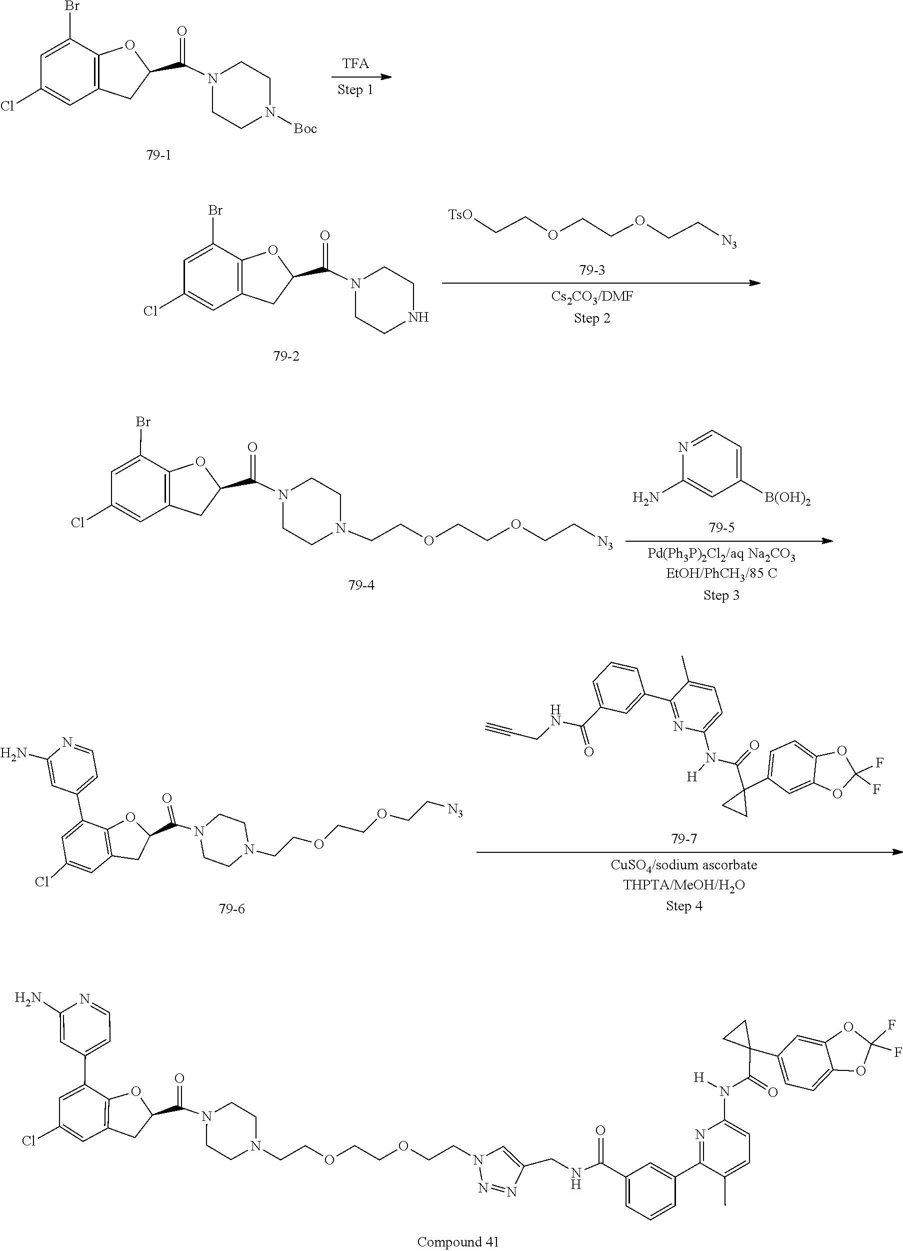

### Example 80. Synthesis of (R)—N-(2-(2-(2-(4-(7-(2-aminopyridin-4-yl)-5-chloro-2,3-dihydrobenzofuran-2-carbonyl)piperazin-1-yl)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 42)

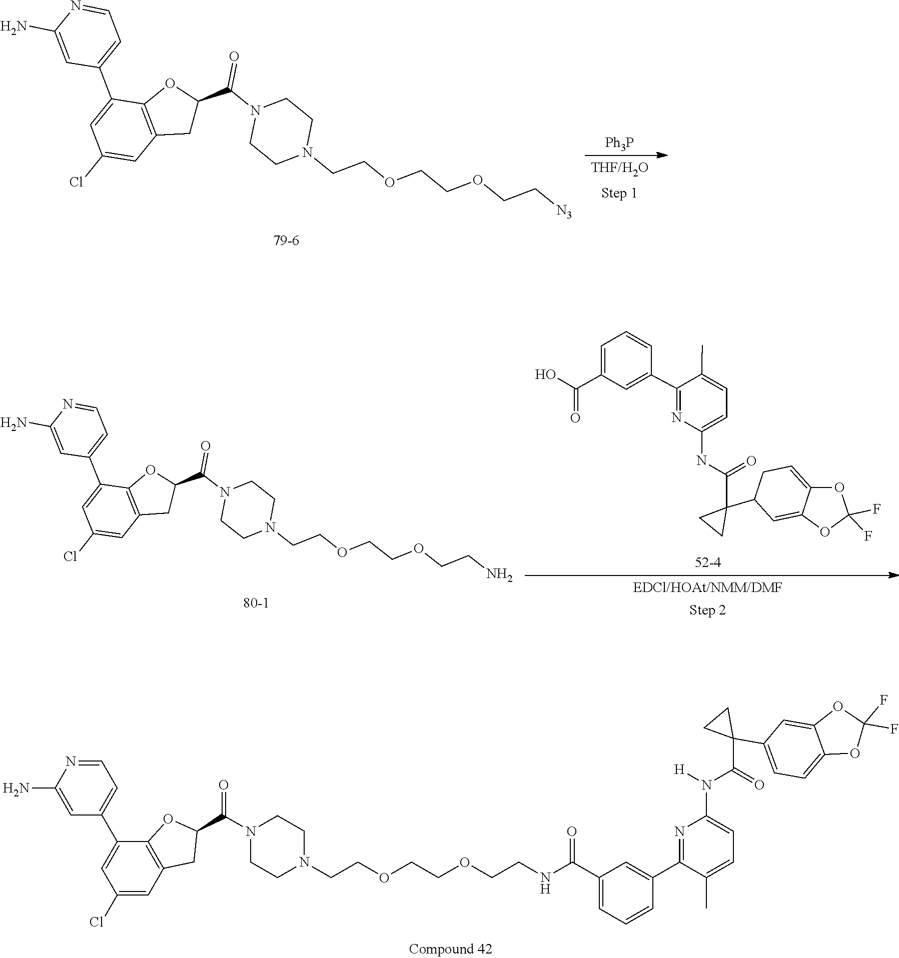

### Example 81. Synthesis of (R)—N-(2-((2-(4-(7-(2-aminopyridin-4-yl)-5-chloro-2,3-dihydrobenzofuran-2-carbonyl)piperazin-1-yl)-2-oxoethyl)thio)ethyl)-3-(6-(1-(2,2-difluorobenzo[d][1,3]dioxol-5-yl)cyclopropane-1-carboxamido)-3-methylpyridin-2-yl)benzamide (Compound 43)

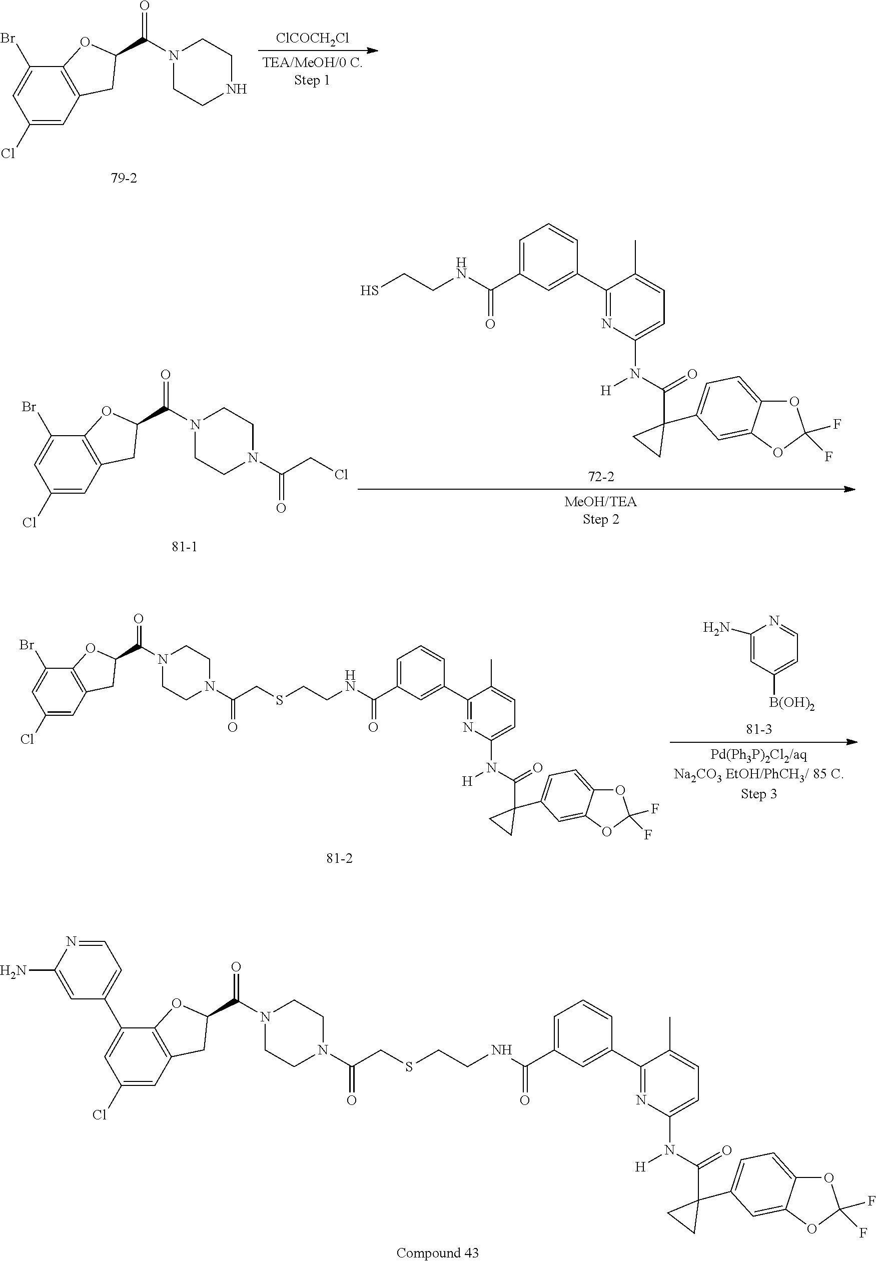

### Example 82. Synthesis of N-(2-((2-(4-((R)-7-(2-aminopyridin-4-yl)-5-chloro-2,3-dihydrobenzofuran-2-carbonyl)piperazin-1-yl)-2-oxoethyl)sulfinyl)ethyl)-3-(6-(1-(2,2-difluorobenzo[d][1,3]dioxol-5-yl)cyclopropane-1-carboxamido)-3-methylpyridin-2-yl)benzamide (Compound 44) and (R)—N-(2-((2-(4-(7-(2-aminopyridin-4-yl)-5-chloro-2,3-dihydrobenzofuran-2-carbonyl)piperazin-1-yl)-2-oxoethyl)sulfonyl)ethyl)-3-(6-(1-(2,2-difluorobenzo[d][1,3]dioxol-5-yl)cyclopropane-1-carboxamido)-3-methylpyridin-2-yl)benzamide (Compound 45)

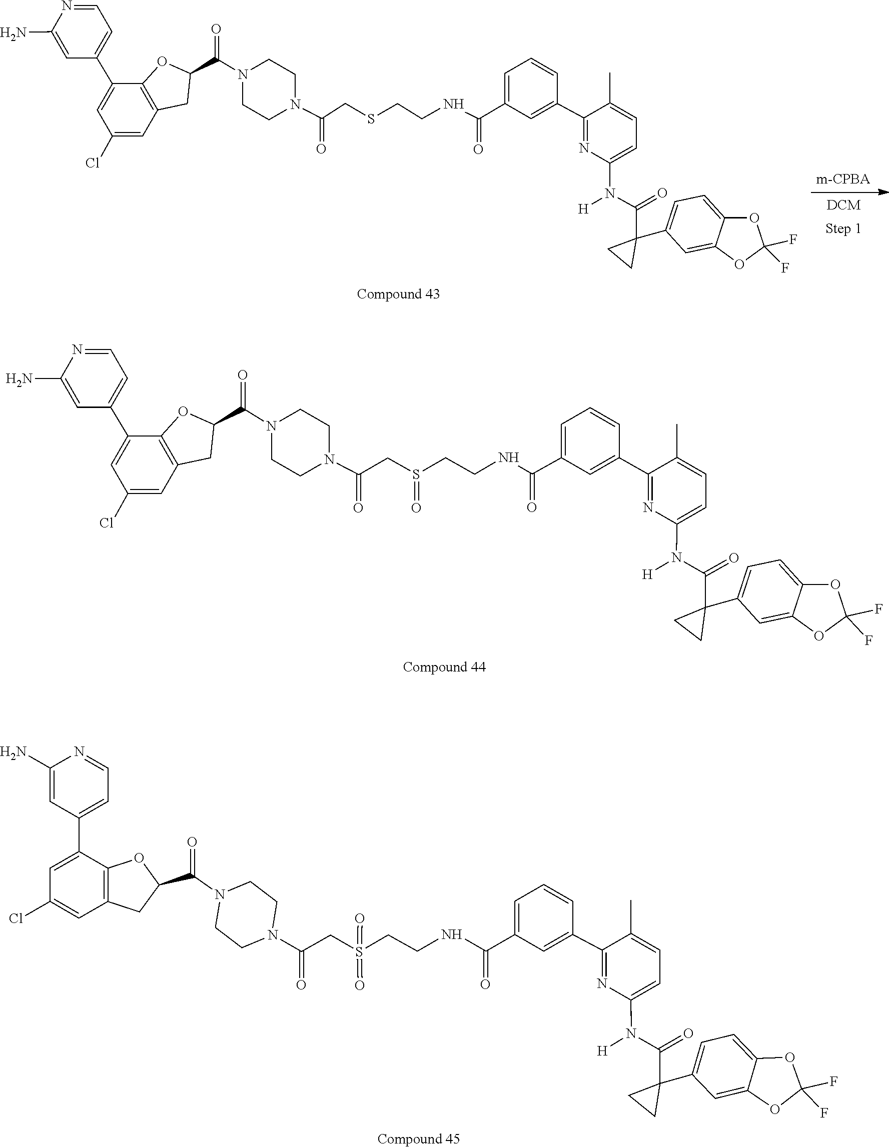 [SMILES: Cc1ccc(NC(=O)C2(c3ccc4c(c3)OC(F)(F)O4)CC2)nc1-c1cccc(C(=O)NCCSCC(=O)N2CCN(C(=O)[C@H]3Cc4cc(Cl)cc(-c5ccnc(N)c5)c4O3)CC2)c1 ; Cc1ccc(NC(=O)C2(c3ccc4c(c3)OC(F)(F)O4)CC2)nc1-c1cccc(C(=O)NCCS(=O)CC(=O)N2CCN(C(=O)[C@H]3Cc4cc(Cl)cc(-c5ccnc(N)c5)c4O3)CC2)c1 ; Cc1ccc(NC(=O)C2(c3ccc4c(c3)OC(F)(F)O4)CC2)nc1-c1cccc(C(=O)NCCS(=O)(=O)CC(=O)N2CCN(C(=O)[C@H]3Cc4cc(Cl)cc(-c5ccnc(N)c5)c4O3)CC2)c1]

### Example 83. Synthesis of (R)—N-(2-(2-(2-(2-(4-(7-(2-aminopyridin-4-yl)-5-chloro-2,3-dihydrobenzofuran-2-carbonyl)piperazin-1-yl)-2-oxoethoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 46)

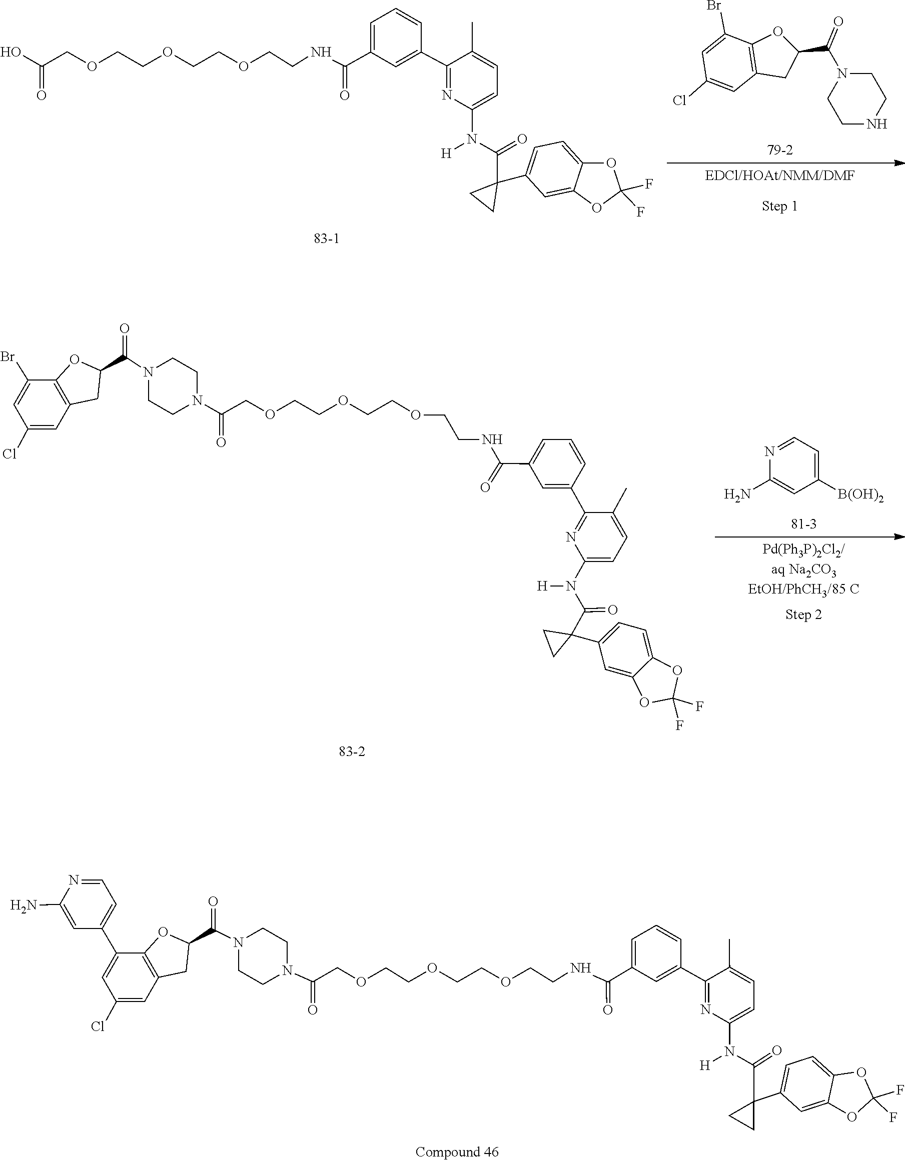 [SMILES: Cc1ccc(NC(=O)C2(c3ccc4c(c3)OC(F)(F)O4)CC2)nc1-c1cccc(C(=O)NCCOCCOCCOCC(=O)O)c1 ; O=C([C@H]1Cc2cc(Cl)cc(Br)c2O1)N1CCNCC1 ; Cc1ccc(NC(=O)C2(c3ccc4c(c3)OC(F)(F)O4)CC2)nc1-c1cccc(C(=O)NCCOCCOCCOCC(=O)N2CCN(C(=O)[C@H]3Cc4cc(Cl)cc(Br)c4O3)CC2)c1 ; Nc1cc(B(O)O)ccn1 ; Cc1ccc(NC(=O)C2(c3ccc4c(c3)OC(F)(F)O4)CC2)nc1-c1cccc(C(=O)NCCOCCOCCOCC(=O)N2CCN(C(=O)[C@H]3Cc4cc(Cl)cc(-c5ccnc(N)c5)c4O3)CC2)c1]

### Example 84. Synthesis of (R)—N-(2-(2-(2-(2-(4-(7-(2-aminopyridin-4-yl)-5-chloro-2,3-dihydrobenzofuran-2-carbonyl)piperazin-1-yl)ethoxy)ethoxy)ethoxy)ethyl)-3-(6-(1-(2,2-difluorobenzo[d][1,3]dioxol-5-yl)cyclopropane-1-carboxamido)-3-methylpyridin-2-yl)benzamide (Compound 47)

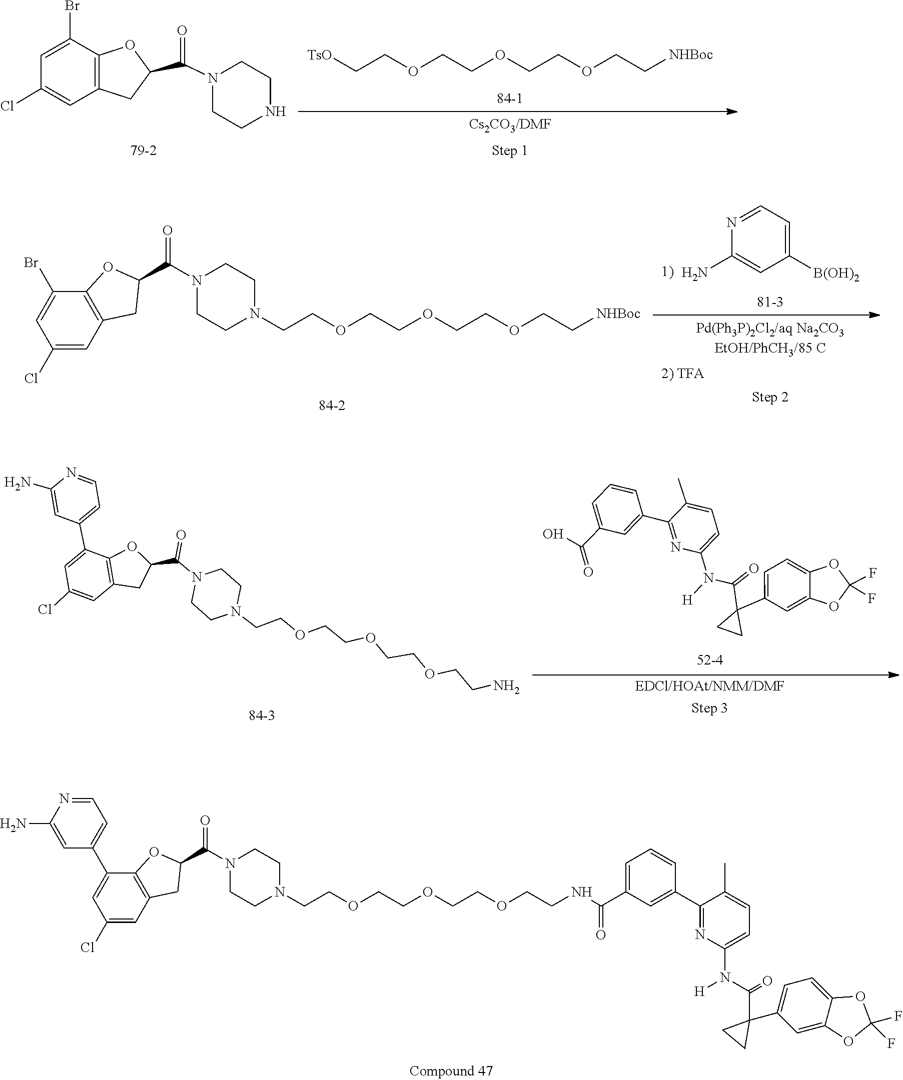

### Example 85. Synthesis of (R)—N-(1-(3-(6-(1-(2,2-difluorobenzo[d][1,3]dioxol-5-yl)cyclopropane-1-carboxamido)-3-methylpyridin-2-yl)phenyl)-1-oxo-5,8,11-trioxa-2-azatridecan-13-yl)-3-((4-hydroxy-1-(3-phenylbutanoyl)piperidin-4-yl)methyl)-4-oxo-3,4-dihydropyrrolo[2,1-f][1,2,4]triazine-7-carboxamide (Compound 48)

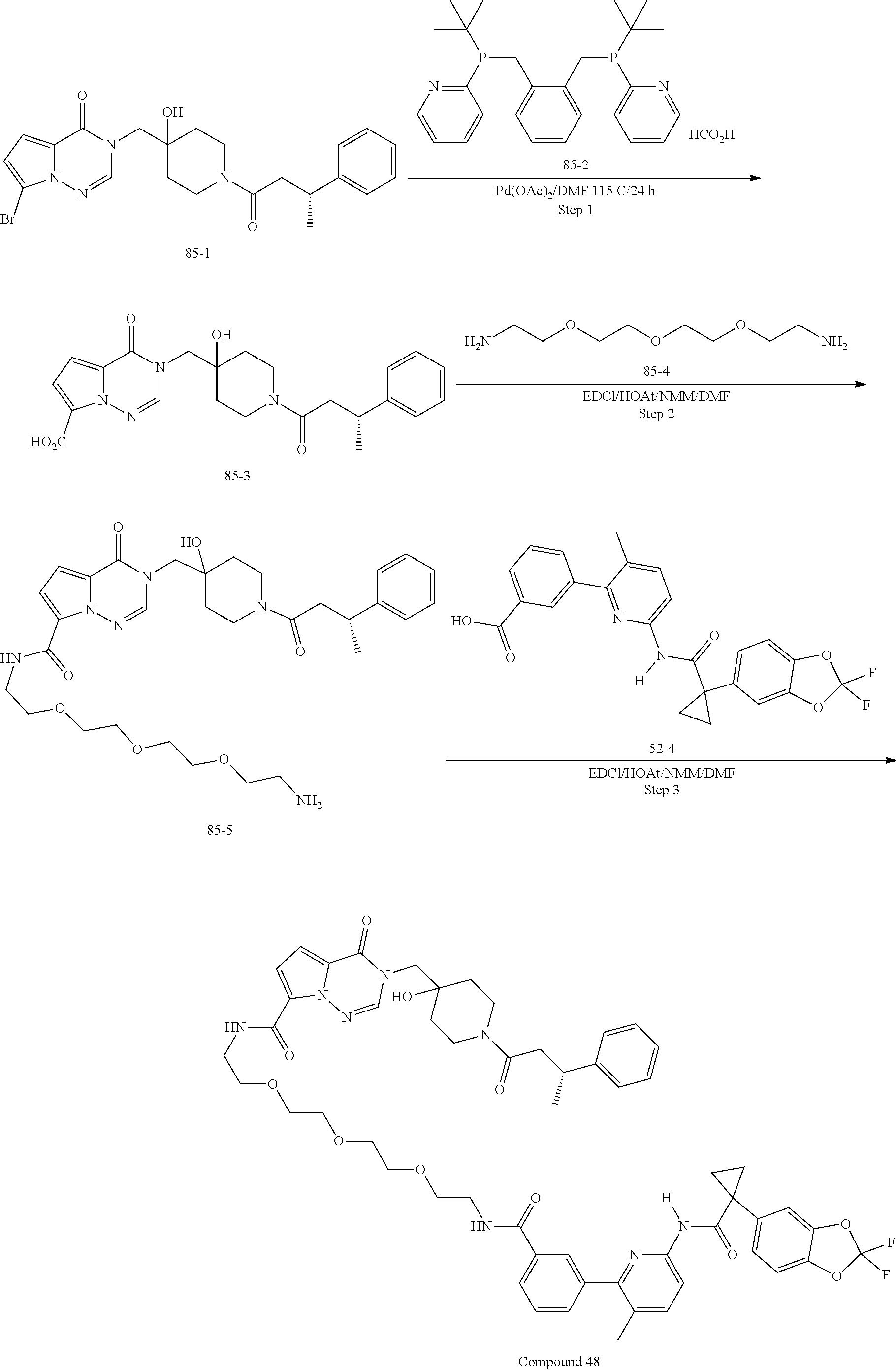

### Example 86. Synthesis of (R)-3-(6-(1-(2,2-difluorobenzo[d][1,3]dioxol-5-yl)cyclopropane-1-carboxamido)-3-methylpyridin-2-yl)-N-(2-(2-(2-((3-((4-hydroxy-1-(3-phenylbutanoyl)piperidin-4-yl)methyl)-4-oxo-3,4-dihydropyrrolo[2,1-f][1,2,4]triazin-7-yl)thio)ethoxy)ethoxy)ethyl)benzamide (Compound 49)

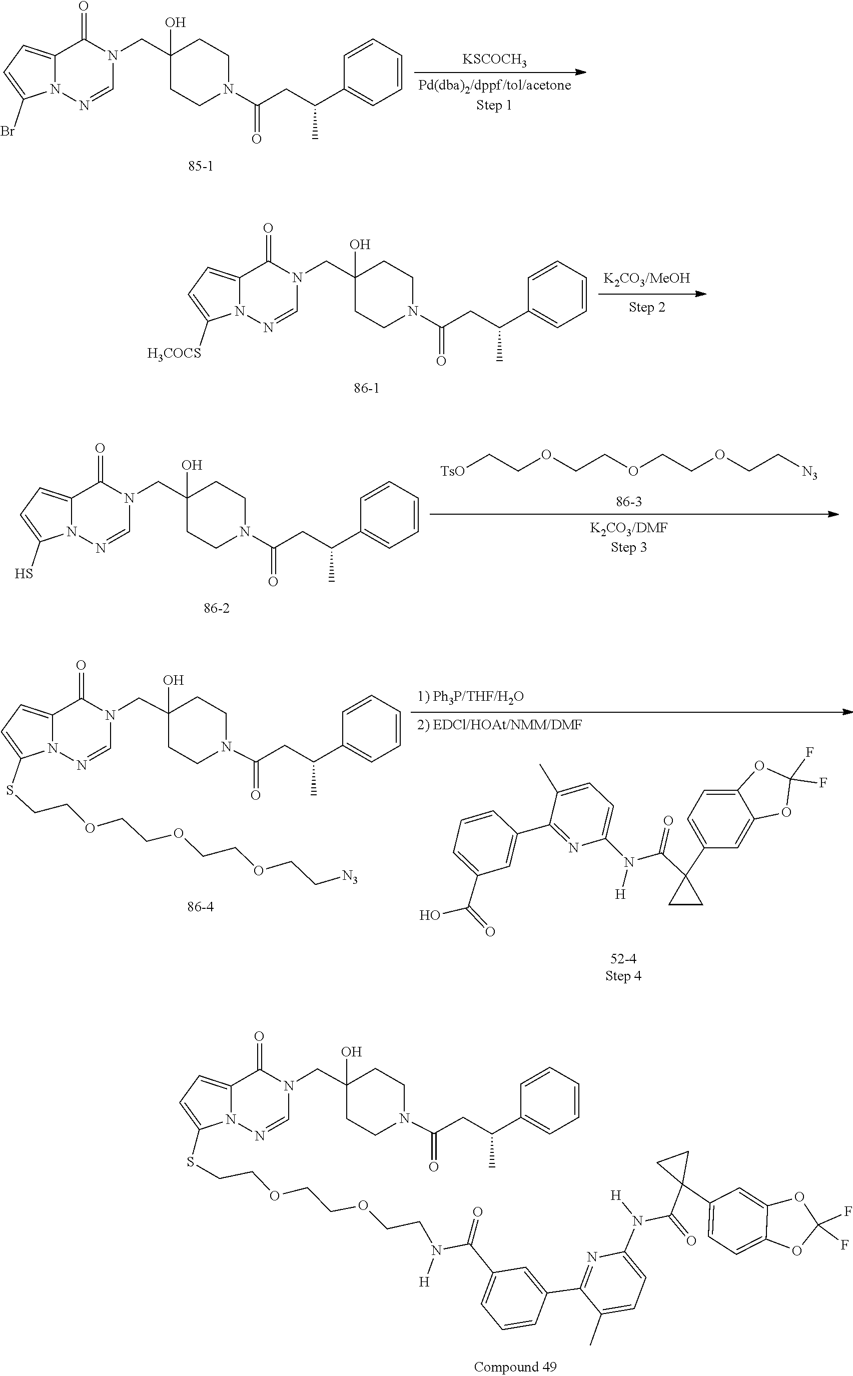

### Example 87. Synthesis of (R)-3-(6-(1-(2,2-difluorobenzo[d][1,3]dioxol-5-yl)cyclopropane-1-carboxamido)-3-methylpyridin-2-yl)-N-(2-(2-(2-((3-(3-((4-hydroxy-1-(3-phenylbutanoyl)piperidin-4-yl)methyl)-4-oxo-3,4-dihydropyrrolo[2,1-f][1,2,4]triazin-7-yl)prop-2-yn-1-yl)oxy)ethoxy)ethoxy)ethyl)benzamide (Compound 50)

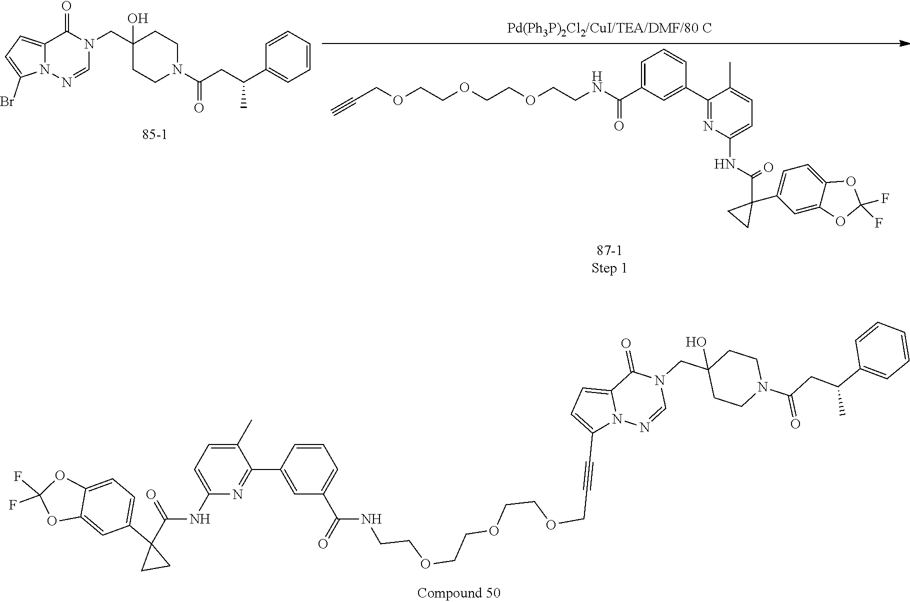 [SMILES: C[C@H](CC(=O)N1CCC(O)(Cn2cnn3c(Br)ccc3c2=O)CC1)c1ccccc1 ; C#CCOCCOCCOCCNC(=O)c1cccc(-c2nc(NC(=O)C3(c4ccc5c(c4)OC(F)(F)O5)CC3)ccc2C)c1 ; Cc1ccc(NC(=O)C2(c3ccc4c(c3)OC(F)(F)O4)CC2)nc1-c1cccc(C(=O)NCCOCCOCCOCC#Cc2ccc3c(=O)n(CC4(O)CCN(C(=O)C[C@@H](C)c5ccccc5)CC4)cnn23)c1]

### Example 88. Synthesis of (R)-3-(6-(1-(2,2-difluorobenzo[d][1,3]dioxol-5-yl)cyclopropane-1-carboxamido)-3-methylpyridin-2-yl)-N-(2-(2-(2-((3-((4-hydroxy-1-(3-phenylbutanoyl)piperidin-4-yl)methyl)-4-oxo-3,4-dihydropyrrolo[2,1-f][1,2,4]triazin-7-yl)amino)-2-oxoethoxy)ethoxy)ethyl)benzamide (Compound 51)

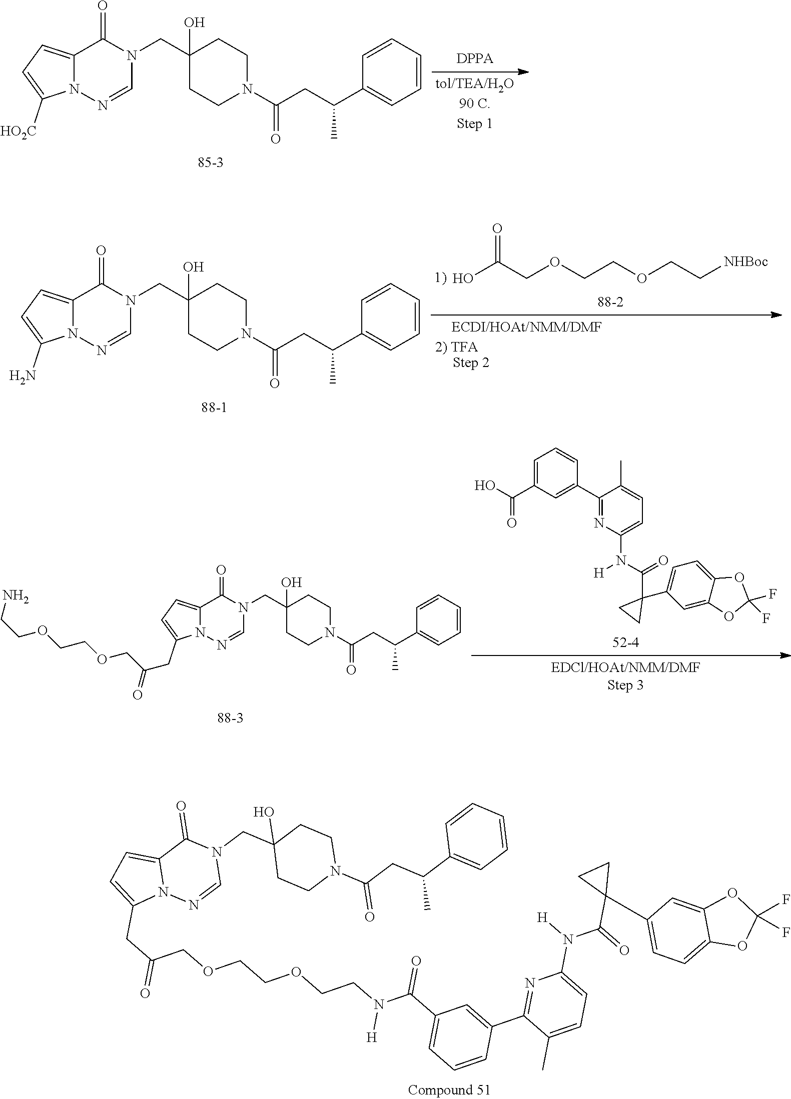 [SMILES: C[C@H](CC(=O)N1CCC(O)(Cn2cnn3c(C(=O)O)ccc3c2=O)CC1)c1ccccc1 ; C[C@H](CC(=O)N1CCC(O)(Cn2cnn3c(N)ccc3c2=O)CC1)c1ccccc1 ; CC(C)(C)OC(=O)NCCOCCOCC(=O)O ; C[C@H](CC(=O)N1CCC(O)(Cn2cnn3c(CC(=O)COCCOCCN)ccc3c2=O)CC1)c1ccccc1 ; Cc1ccc(NC(=O)C2(c3ccc4c(c3)OC(F)(F)O4)CC2)nc1-c1cccc(C(=O)O)c1 ; Cc1ccc(NC(=O)C2(c3ccc4c(c3)OC(F)(F)O4)CC2)nc1-c1cccc(C(=O)NCCOCCOCC(=O)Cc2ccc3c(=O)n(CC4(O)CCN(C(=O)C[C@@H](C)c5ccccc5)CC4)cnn23)c1]

### Example 89. Synthesis of (R)-3-(6-(1-(2,2-difluorobenzo[d][1,3]dioxol-5-yl)cyclopropane-1-carboxamido)-3-methylpyridin-2-yl)-N-(2-(2-(2-((3-((4-hydroxy-1-(3-phenylbutanoyl)piperidin-4-yl)methyl)-4-oxo-3,4-dihydropyrrolo[2,1-f][1,2,4]triazin-7-yl)oxy)ethoxy)ethoxy)ethyl)benzamide (Compound 52)

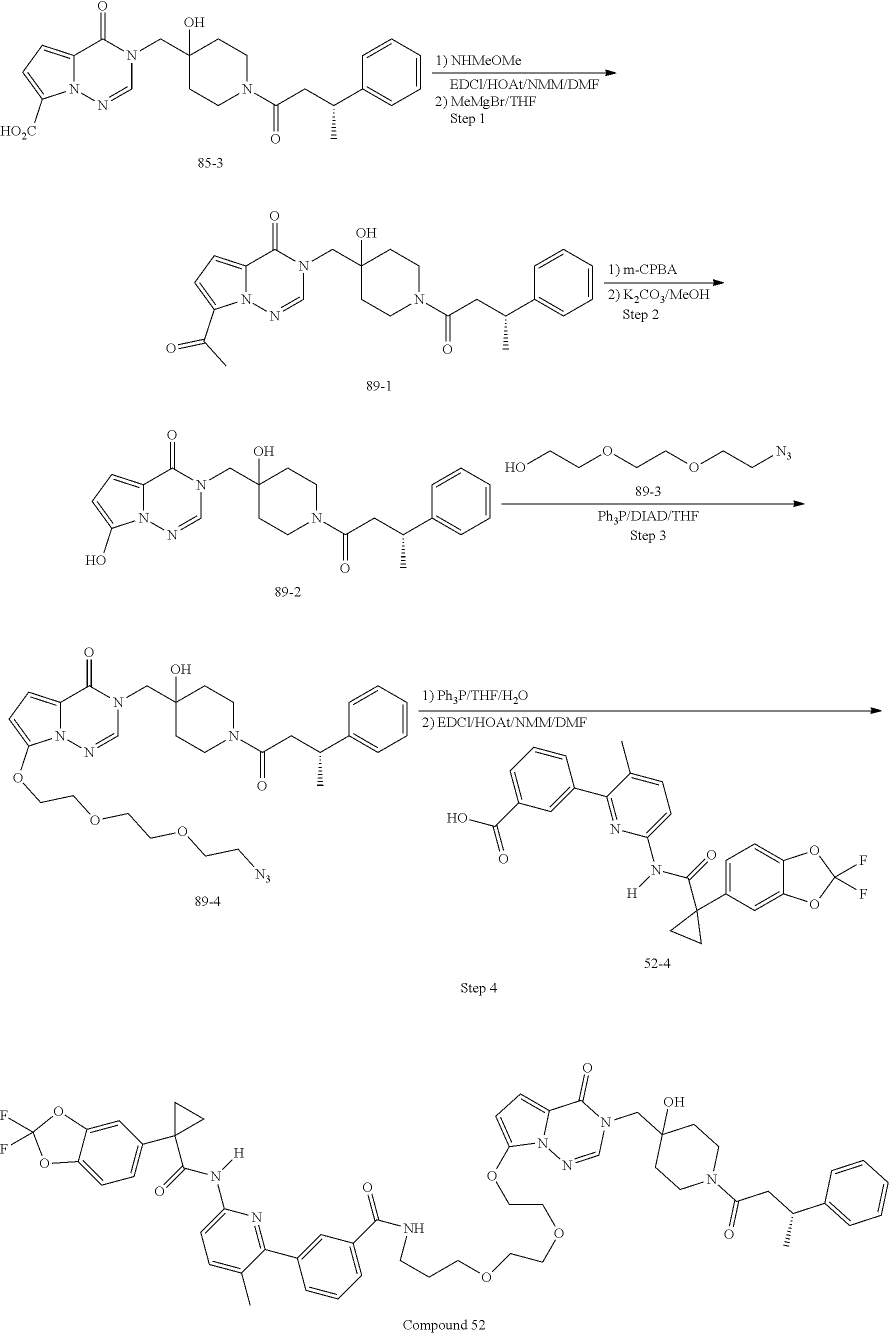

### Example 90. Synthesis of (R)-3-(6-(1-(2,2-difluorobenzo[d][1,3]dioxol-5-yl)cyclopropane-1-carboxamido)-3-methylpyridin-2-yl)-N-(2-(2-(2-((4-(3-((4-hydroxy-1-(3-phenylbutanoyl)piperidin-4-yl)methyl)-4-oxo-3,4-dihydropyrrolo[2,1-f][1,2,4]triazin-7-yl)benzyl)amino)-2-oxoethoxy)ethoxy)ethyl)benzamide (Compound 53)

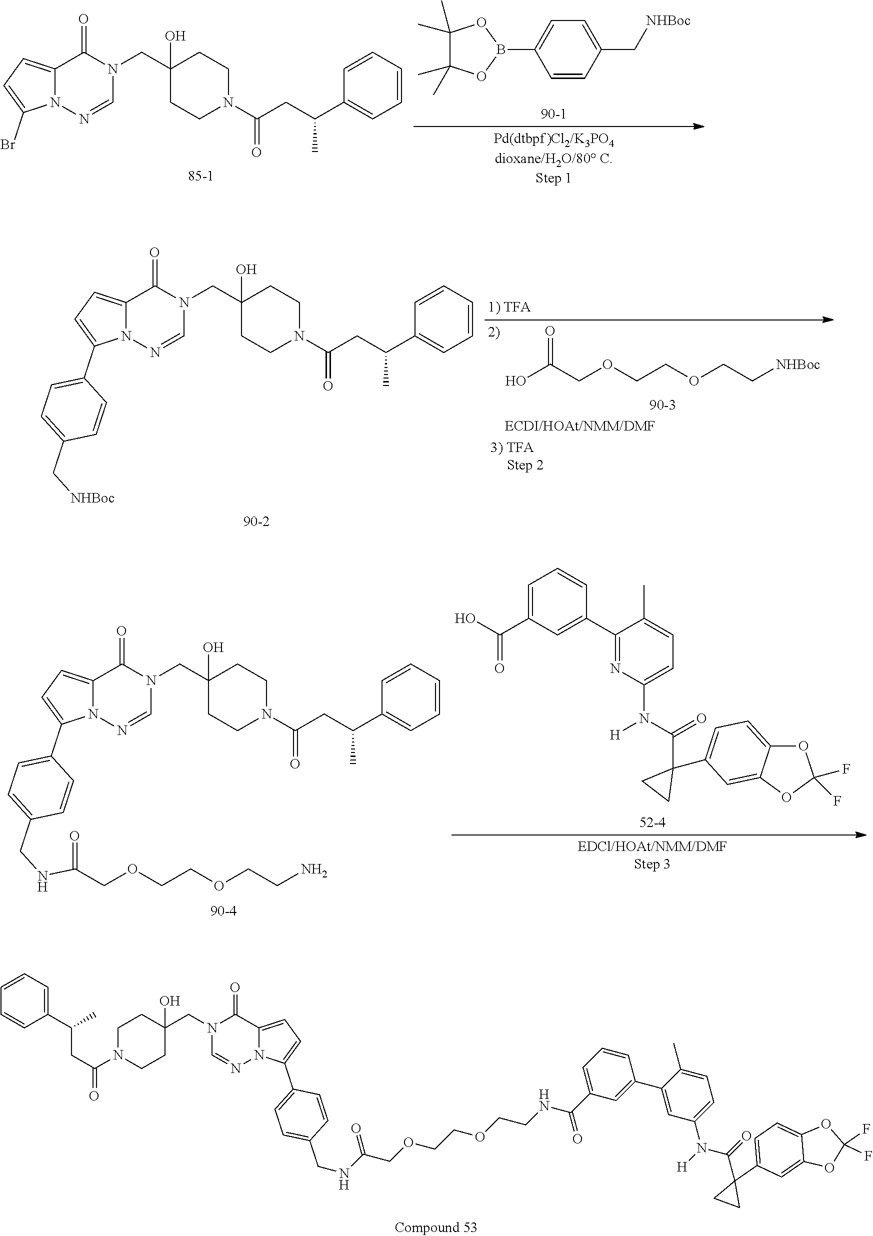

### Example 91. Synthesis of 4-(2-amino-5-(1-(2-aminoethyl)-1H-indazol-5-yl)-4-ethylpyridin-3-yl)phenol (Intermediate 91-6)

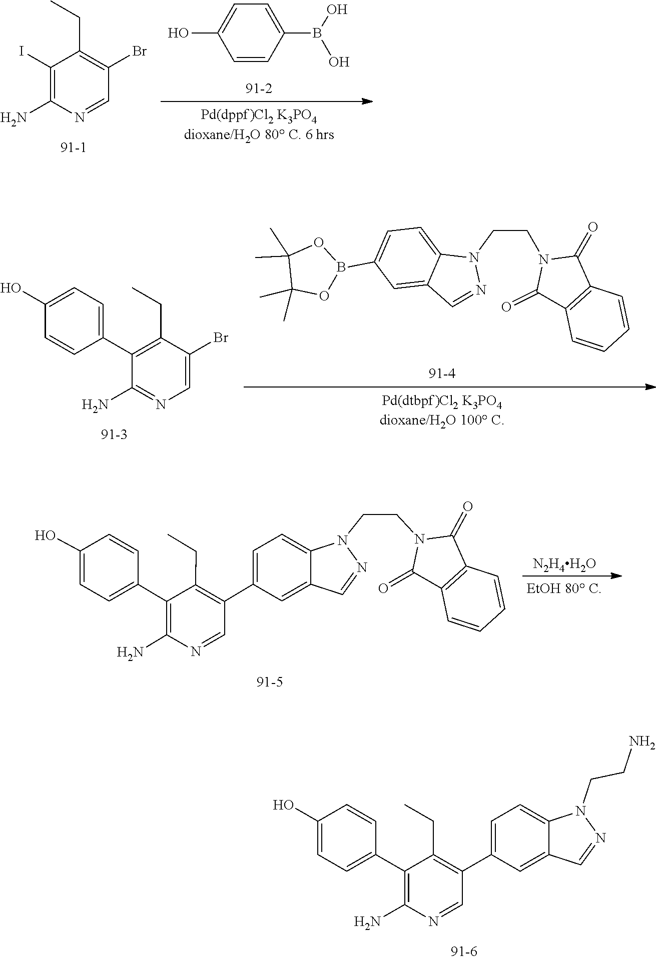

### Example 92. Synthesis of tert-butyl ((4-ethyl-6-formyl-3-(4-hydroxyphenyl)-5-(1H-indazol-5-yl)pyridin-2-yl)methyl)carbamate

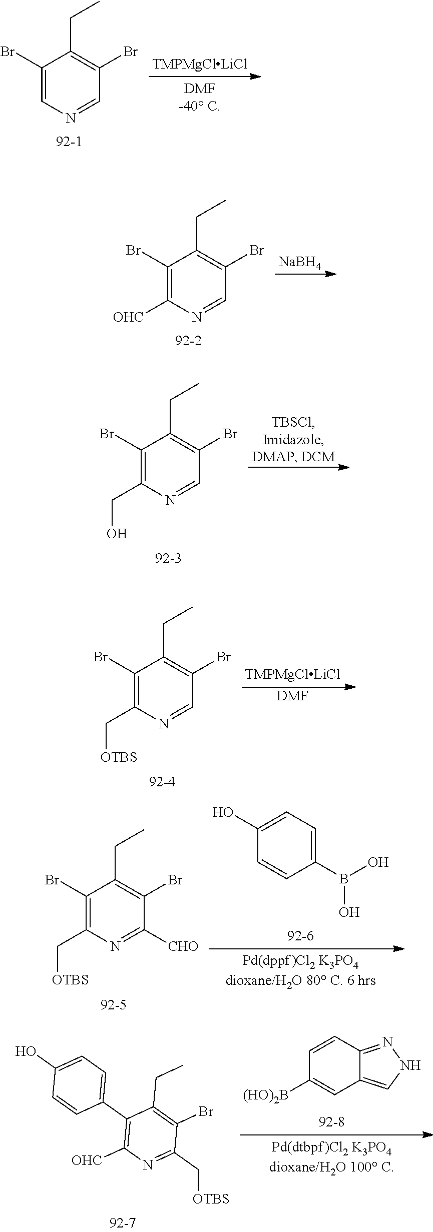

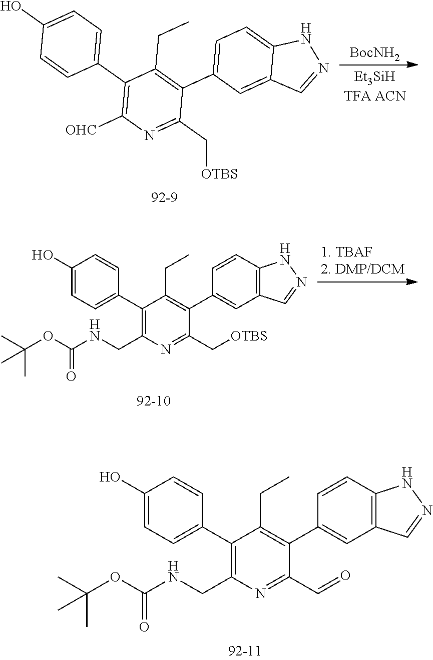

### Example 93 Synthesis of tert-butyl ((4-ethyl-6-formyl-3-(4-hydroxyphenyl)-5-(1H-indazol-5-yl)pyridin-2-yl)methyl)carbamate

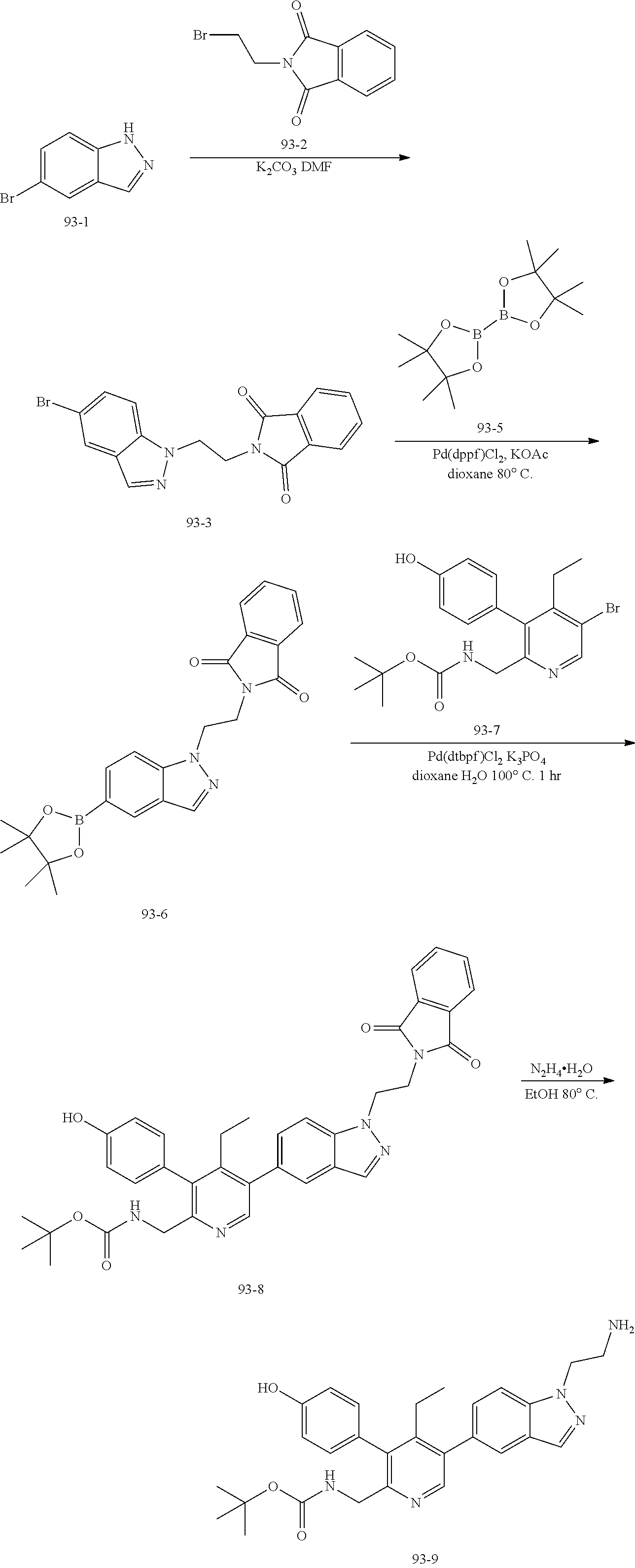

### Example 94 Synthesis of 3-(4-((6-(thiophene-2-sulfonamido)benzo[d]thiazol-2-yl)carbamoyl)piperidin-1-yl)propanoic acid

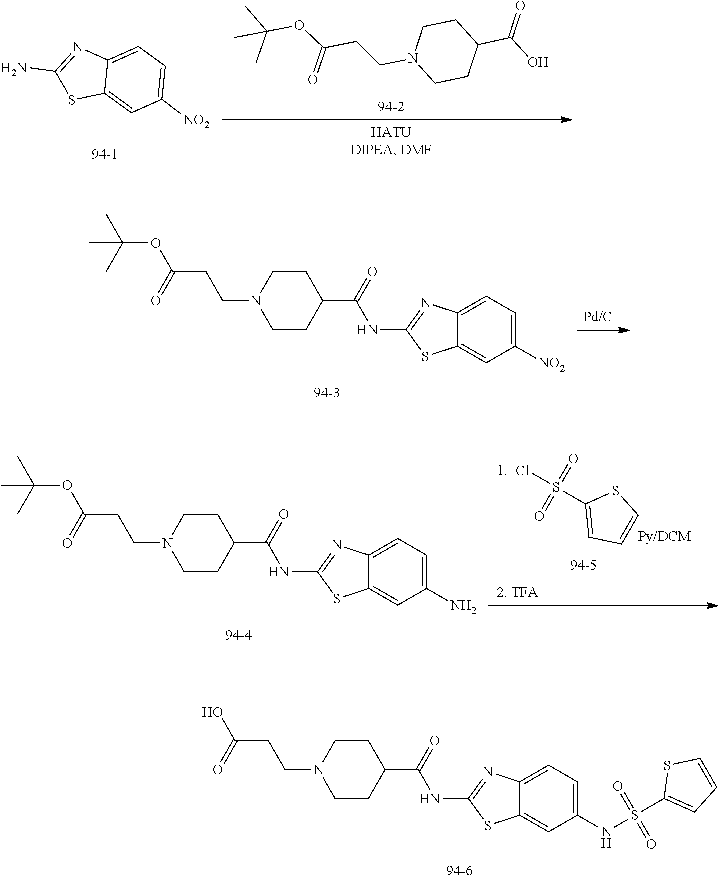 [SMILES: Nc1nc2ccc([N+](=O)[O-])cc2s1 ; CC(C)(C)OC(=O)CCN1CCC(C(=O)O)CC1 ; CC(C)(C)OC(=O)CCN1CCC(C(=O)Nc2nc3ccc([N+](=O)[O-])cc3s2)CC1 ; CC(C)(C)OC(=O)CCN1CCC(C(=O)Nc2nc3ccc(N)cc3s2)CC1 ; O=S(=O)(Cl)c1cccs1 ; O=C(O)CCN1CCC(C(=O)Nc2nc3ccc(NS(=O)(=O)c4cccs4)cc3s2)CC1]

### Example 95 Synthesis of 3-(4-((6-(thiophene-2-sulfonamido)benzo[d]thiazol-2-yl)carbamoyl)piperidin-1-yl)propanoic acid

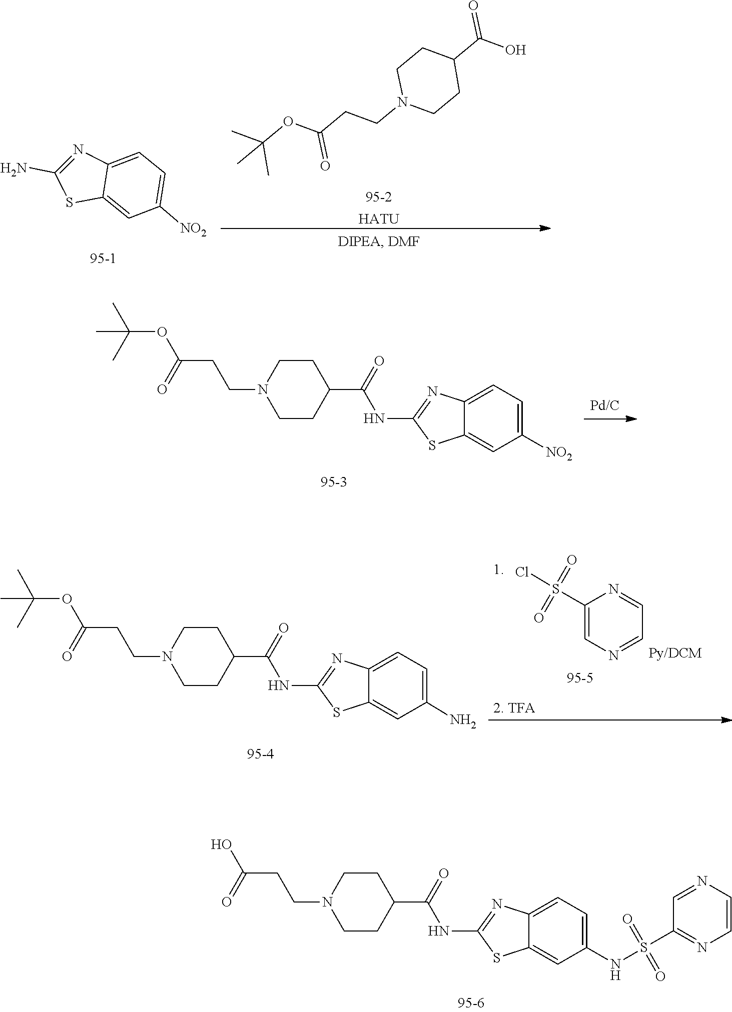

### Example 96 Synthesis of 3-(4-((6-((3-methylisoxazole)-4-sulfonamido)benzo[d]thiazol-2-yl)carbamoyl)piperidin-1-yl)propanoic acid

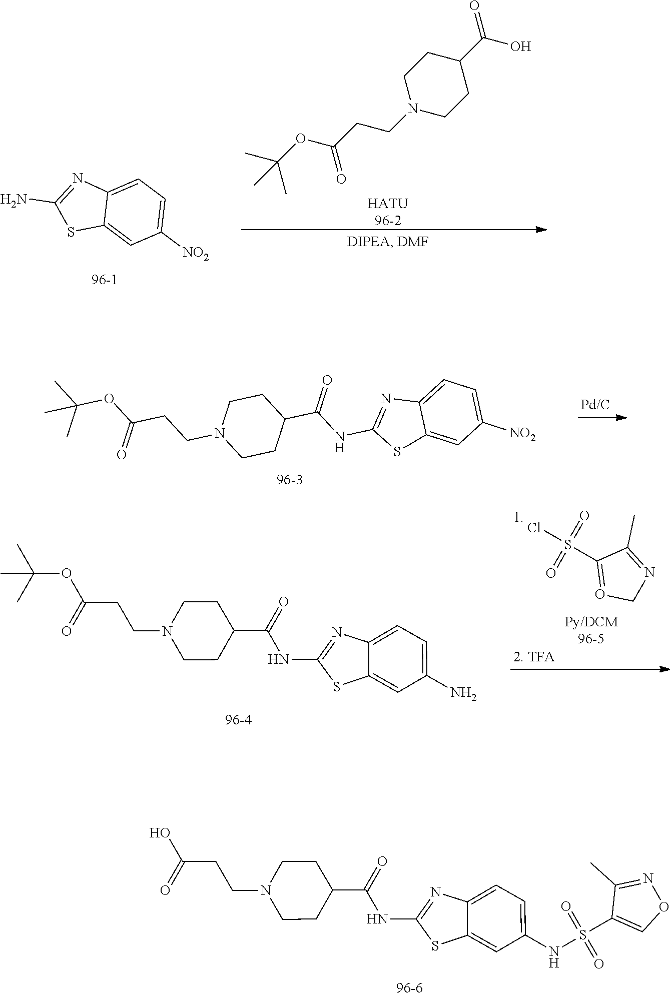

### Example 97 Synthesis of 1-(2-aminoethyl)-N-(4-fluoro-6-(thiophene-2-sulfonamido)benzo[d]thiazol-2-yl)piperidine-4-carboxamide hydrochloride

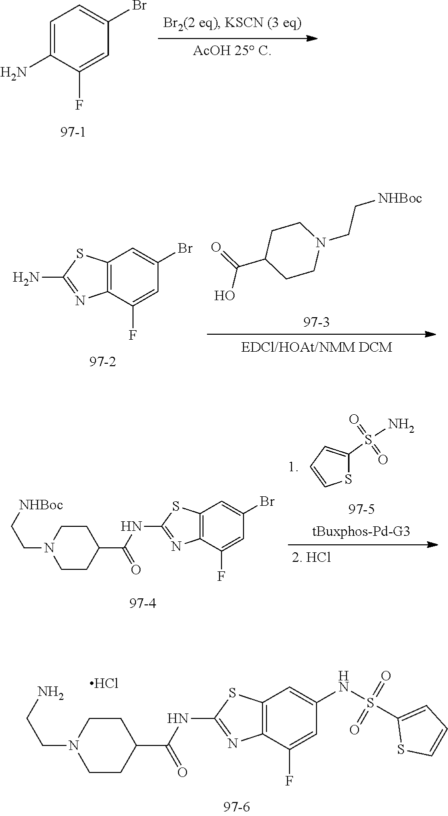

### Example 98 Synthesis of 1-(2-aminoethyl)-N-(5-fluoro-6-(thiophene-2-sulfonamido)benzo[d]thiazol-2-yl)piperidine-4-carboxamide hydrochloride & 1-(2-aminoethyl)-N-(7-fluoro-6-(thiophene-2-sulfonamido)benzo[d]thiazol-2-yl)piperidine-4-carboxamide hydrochloride

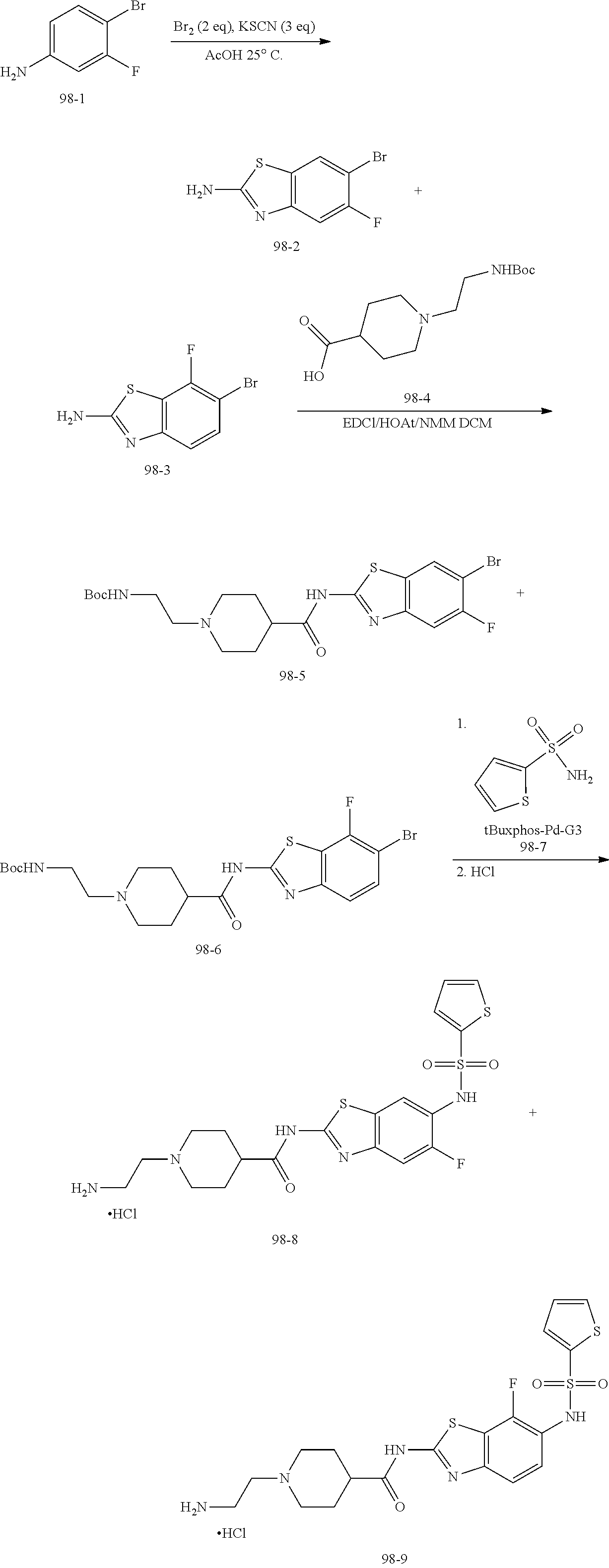

### Example 99 Synthesis of N-(1-(2-(piperidin-4-yl)ethyl)piperidin-4-yl)-6-(thiophene-2-sulfonamido)benzo[d]thiazole-2-carboxamide

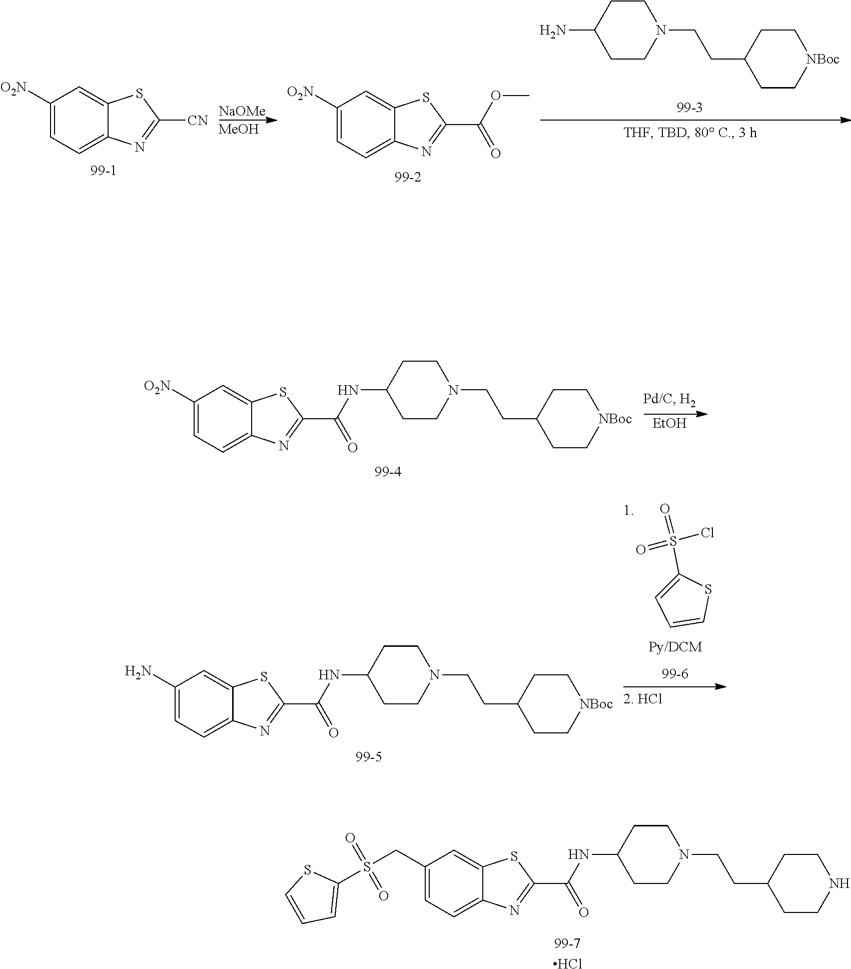

### Scheme 100 Synthesis of 1-(2-(1H-pyrazol-4-yl)ethyl)-N-(6-(thiophene-2-sulfonamido)thiazolo[4,5-c]pyridin-2-yl)piperidine-4-carboxamide

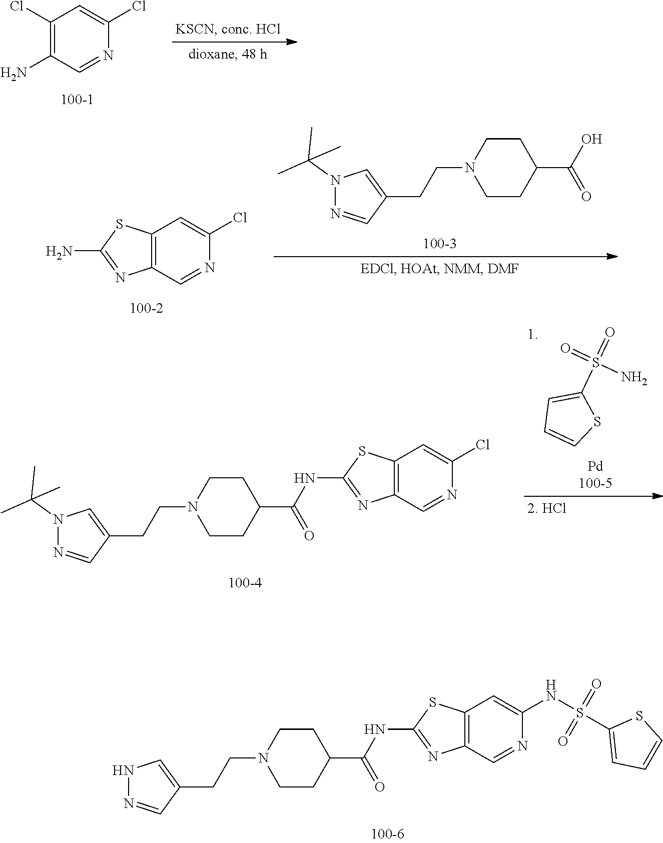 [SMILES: Nc1cnc(Cl)cc1Cl ; Nc1nc2cnc(Cl)cc2s1 ; CC(C)(C)n1cc(CCN2CCC(C(=O)O)CC2)cn1 ; CC(C)(C)n1cc(CCN2CCC(C(=O)Nc3nc4cnc(Cl)cc4s3)CC2)cn1 ; NS(=O)(=O)c1cccs1 ; O=C(Nc1nc2cnc(NS(=O)(=O)c3cccs3)cc2s1)C1CCN(CCc2cn[nH]c2)CC1]

### Example 101. Synthesis of 3-(4-((6-(pyridine-4-sulfonamido)benzo[d]oxazol-2-yl)carbamoyl)piperidin-1-yl)propanoic acid

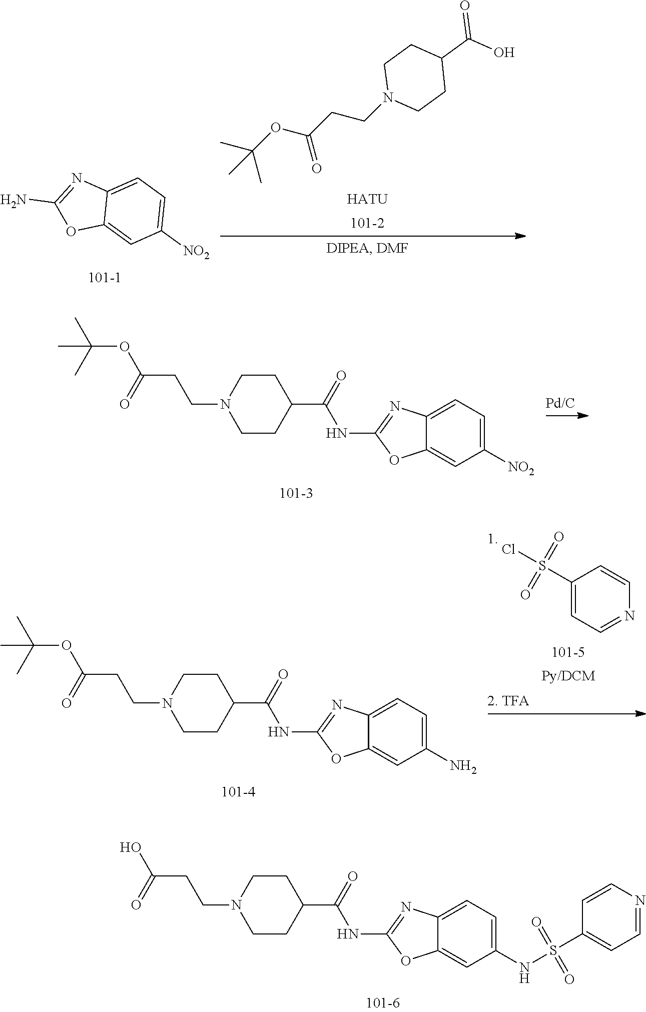

### Example 102. Synthesis of 3-(4-((5-(pyridine-2-sulfonamido)benzo[d]thiazol-2-yl)carbamoyl)piperidin-1-yl)propanoic acid

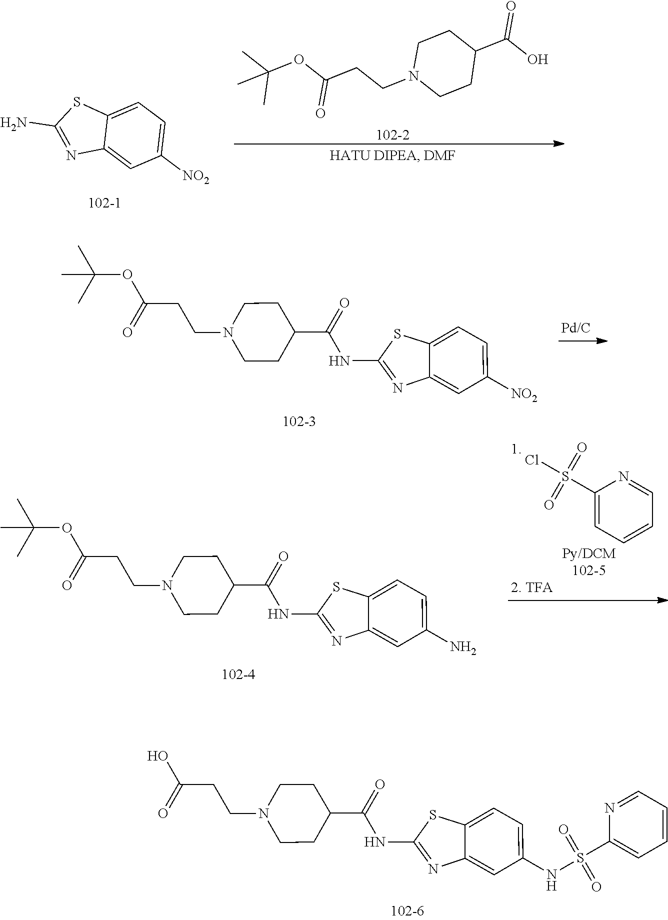

### Example 103. Synthesis of 1-(2-(1H-pyrazol-4-yl)ethyl)-N-(6-(thiophene-2-sulfonamido)thiazolo[4,5-b]pyridin-2-yl)piperidine-4-carboxamide

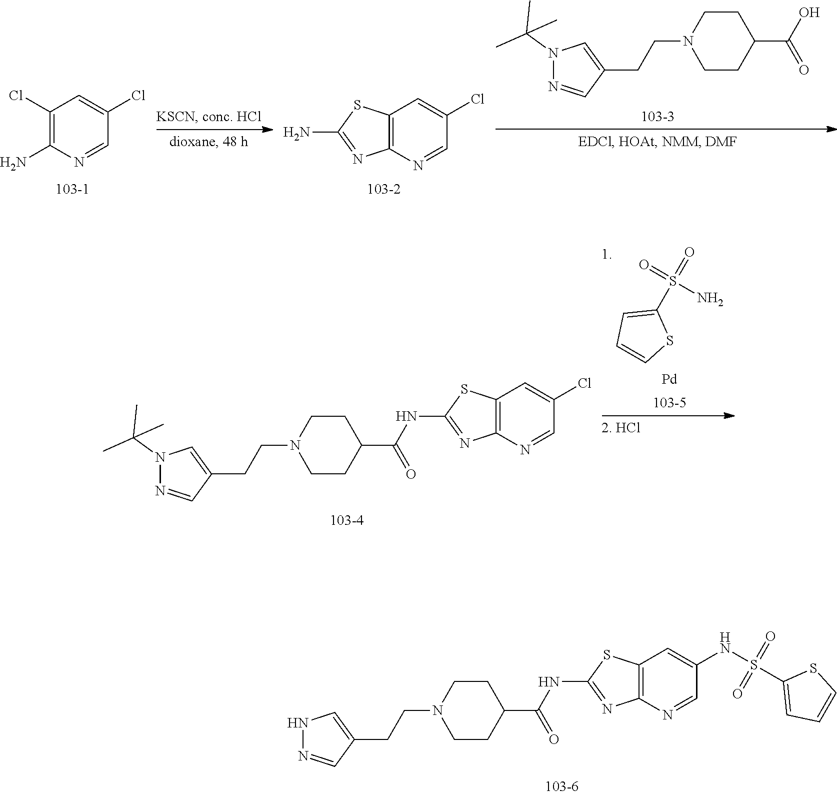

### Example 104. Synthesis of 1-(2-(1H-pyrazol-4-yl)ethyl)-N-(5-(thiophene-2-sulfonamido)thiazolo[5,4-b]pyridin-2-yl)piperidine-4-carboxamide

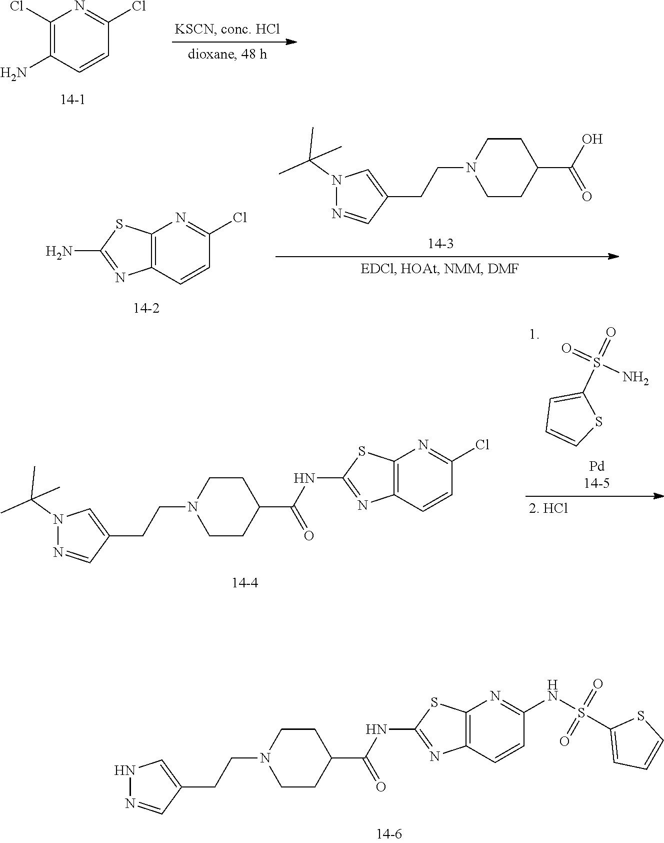 [SMILES: Nc1ccc(Cl)nc1Cl ; Nc1nc2ccc(Cl)nc2s1 ; CC(C)(C)n1cc(CCN2CCC(C(=O)O)CC2)cn1 ; CC(C)(C)n1cc(CCN2CCC(C(=O)Nc3nc4ccc(Cl)nc4s3)CC2)cn1 ; NS(=O)(=O)c1cccs1 ; O=C(Nc1nc2ccc(NS(=O)(=O)c3cccs3)nc2s1)C1CCN(CCc2cn[nH]c2)CC1]

### Example 105. Synthesis of 1-(2-aminoethyl)-N-(6-(N-(2-(dimethylamino)ethyl)thiophene-2-sulfonamido)-4-fluorobenzo[d]thiazol-2-yl)piperidine-4-carboxamide dihydrochloride

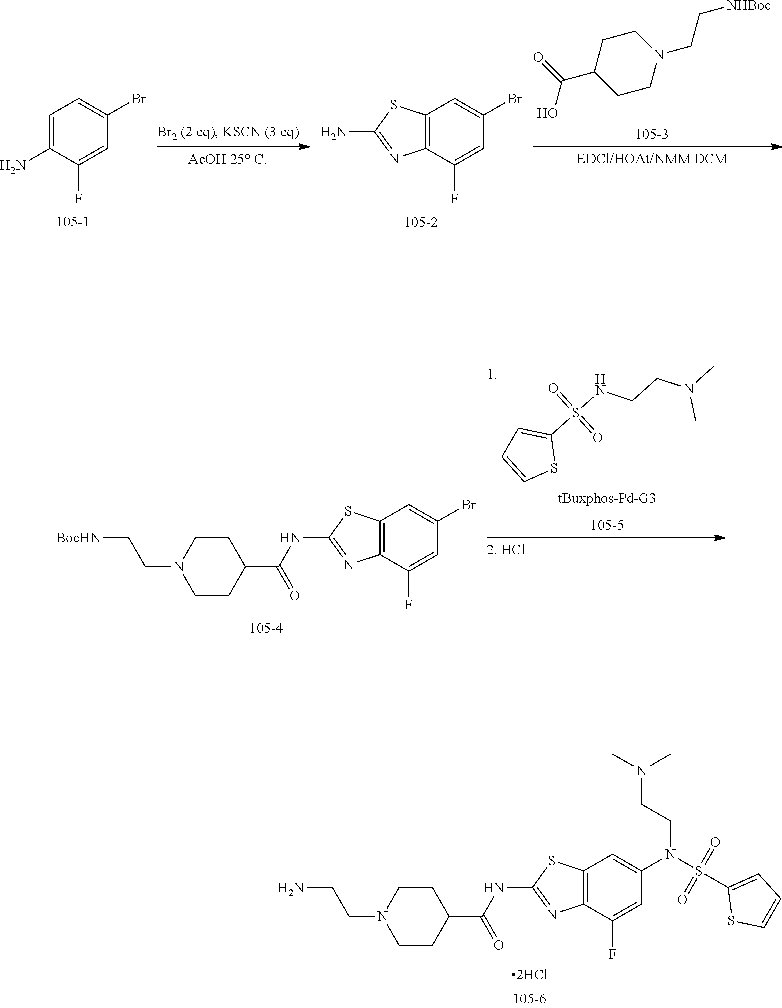

### Example 106. Synthesis of 1-(2-aminoethyl)-N-(6-(N-(2-hydroxyethyl)thiophene-2-sulfonamido)benzo[d]thiazol-2-yl)piperidine-4-carboxamide hydrochloride

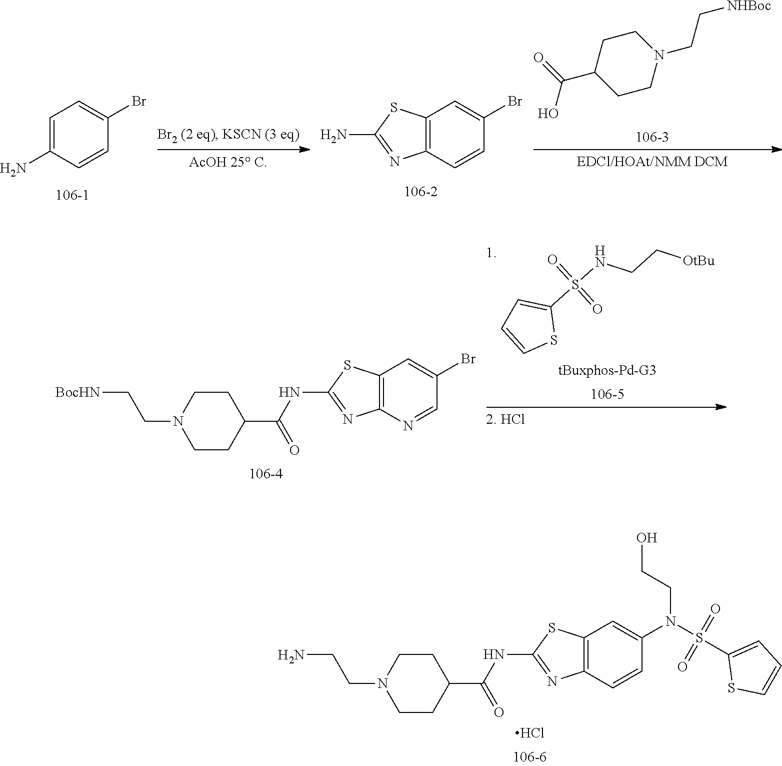

### Example 107. Synthesis of N-(2-((5-(aminomethyl)pyridin-2-yl)amino)benzo[d]thiazol-6-yl)thiophene-2-sulfonamide hydrochloride

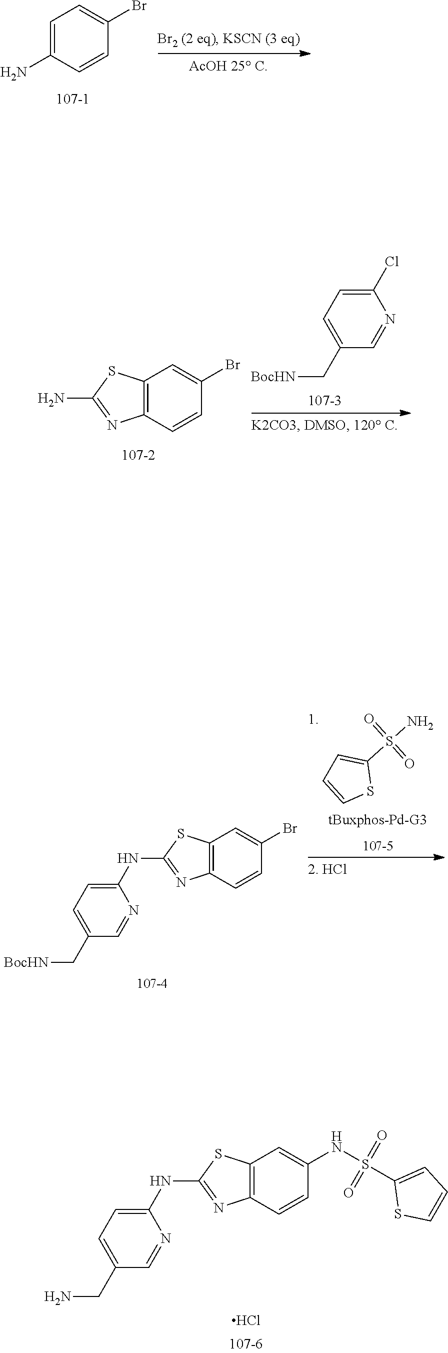

### Example 108. Synthesis of 5-(piperidin-4-ylmethyl)-N-(6-(thiophene-2-sulfonamido)benzo[d]thiazol-2-yl)-5,6,7,8-tetrahydro-4H-pyrazolo[1,5-a][1,4]diazepine-2-carboxamide hydrochloride

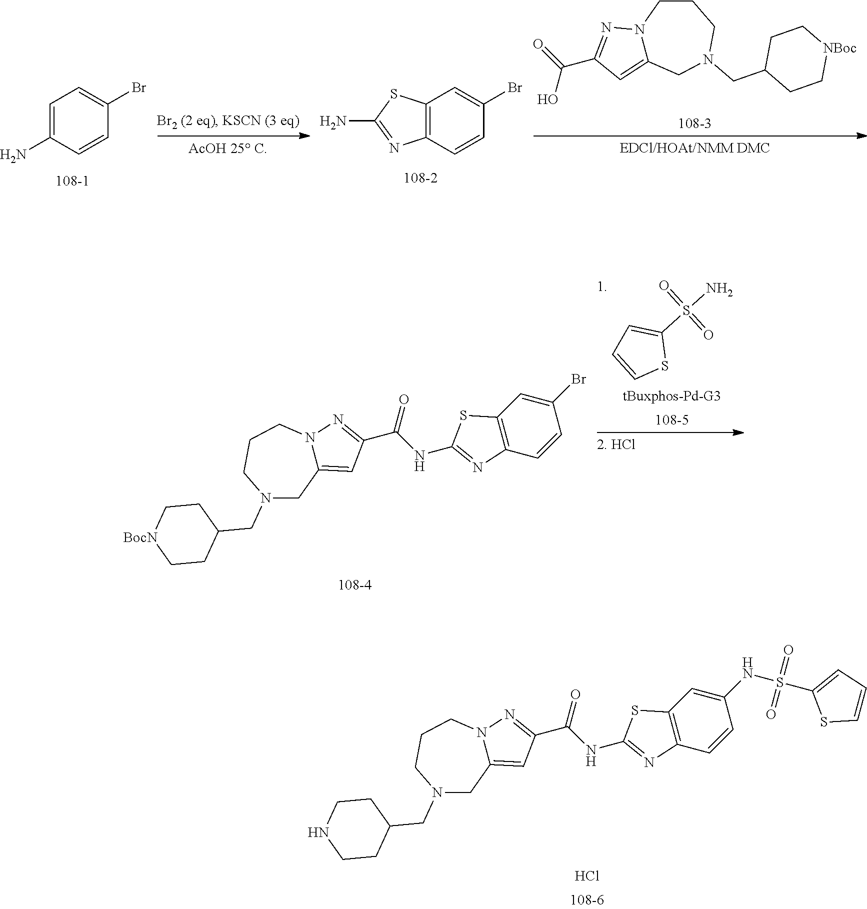

### Example 109. Synthesis of 2-(2-(5-(4,4,5,5-tetramethyl-1,3,2-dioxaborolan-2-yl)-1H-indazol-1-yl)ethyl)isoindoline-1,3-dione

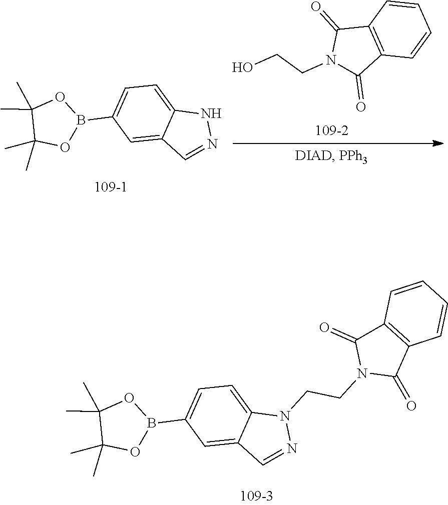 [SMILES: CC1(C)OB(c2ccc3[nH]ncc3c2)OC1(C)C ; O=C1c2ccccc2C(=O)N1CCO ; CC1(C)OB(c2ccc3c(cnn3CCN3C(=O)c4ccccc4C3=O)c2)OC1(C)C]

### Example 110. Synthesis of 1-(3-(tert-butoxy)-3-oxopropyl)piperidine-4-carboxylic acid

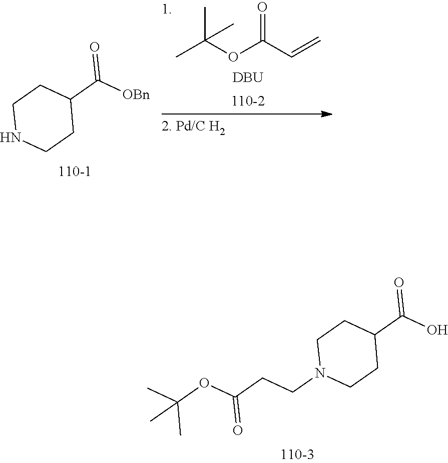

### Example 111. Synthesis of tert-butyl 4-(2-(4-aminopiperidin-1-yl)ethyl)piperidine-1-carboxylate

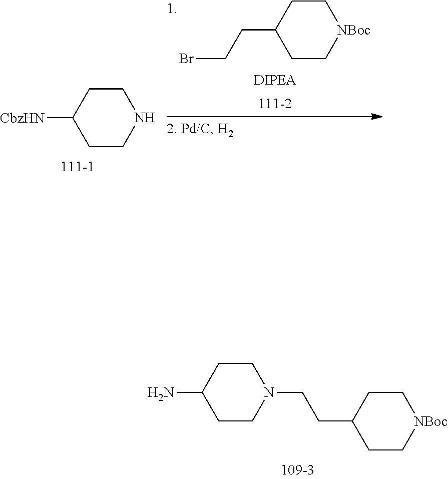

### Example 112. Synthesis of N-(2-(tert-butoxy)ethyl)thiophene-2-sulfonamide

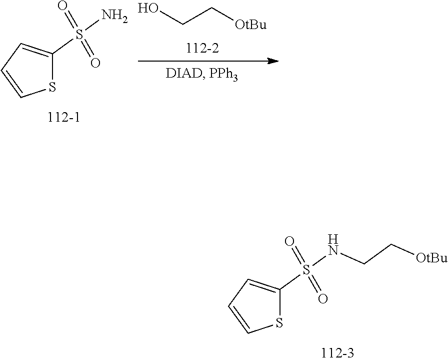 [SMILES: NS(=O)(=O)c1cccs1 ; CC(C)(C)OCCO ; CC(C)(C)OCCNS(=O)(=O)c1cccs1]

### Example 113. Synthesis of 1-(2-(1-(tert-butyl)-1H-pyrazol-4-yl)ethyl)piperidine-4-carboxylic acid

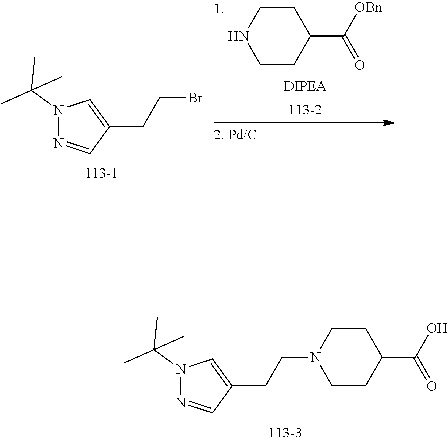

### Example 114. Synthesis of N-(2-(dimethylamino)ethyl)thiophene-2-sulfonamide

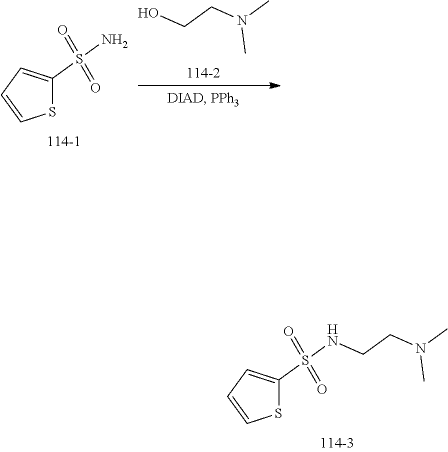

### Example 115. Synthesis of 1-(2-((tert-butoxycarbonyl)amino)ethyl)piperidine-4-carboxylic acid

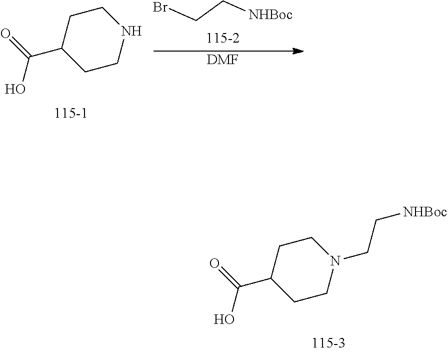

### Example 116. Synthesis of 5-((1-(tert-butoxycarbonyl)piperidin-4-yl)methyl)-5,6,7,8-tetrahydro-4H-pyrazolo[1,5-a][1,4]diazepine-2-carboxylic acid

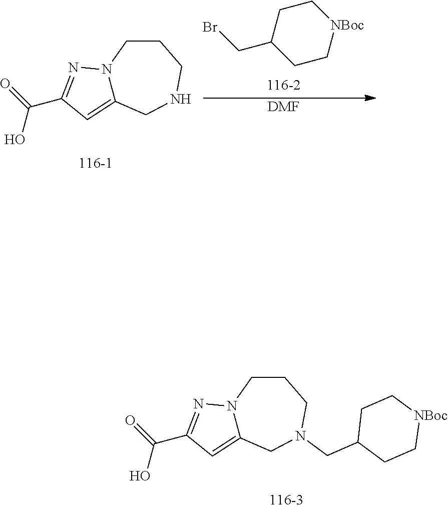

### Example 117. Synthesis of N-(2-(2-(2-((2-(5-(6-amino-4-ethyl-5-(4-hydroxyphenyl)pyridin-3-yl)-1H-indazol-1-yl)ethyl)amino)-2-oxoethoxy)ethoxy)ethyl)-3-(6-(1-(2,2-difluorobenzo[d][1,3]dioxol-5-yl)cyclopropane-1-carboxamido)-3-methylpyridin-2-yl)benzamide Compound 54

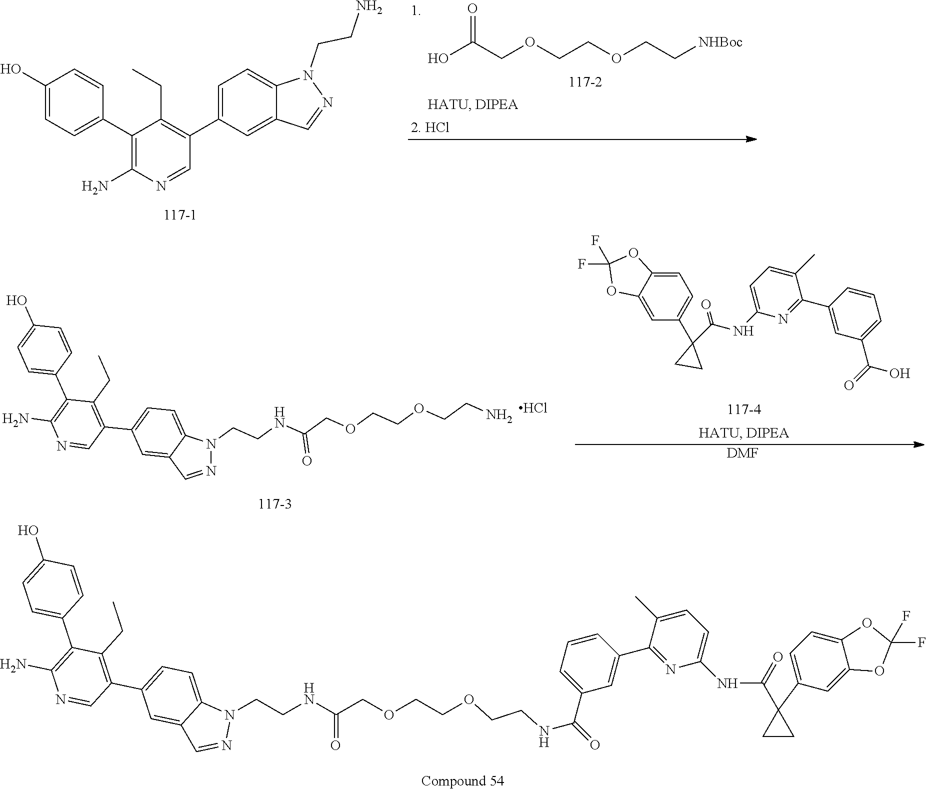

### Example 118. Synthesis of tert-butyl ((6-(12-(3-(6-(1-(2,2-difluorobenzo[d][1,3]dioxol-5-yl)cyclopropane-1-carboxamido)-3-methylpyridin-2-yl)phenyl)-12-oxo-5,8-dioxa-2,11-diazadodecyl)-4-ethyl-3-(4-hydroxyphenyl)-5-(1H-indazol-5-yl)pyridin-2-yl)methyl)carbamate Compound 55

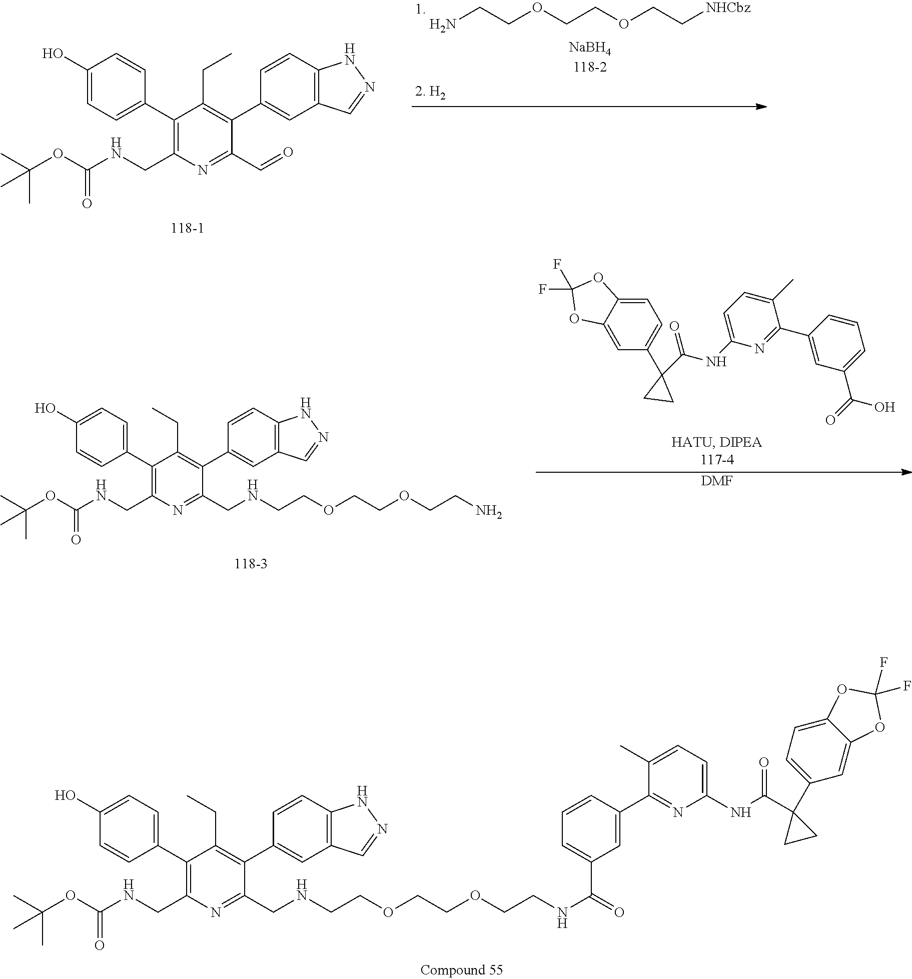

### Example 119. Synthesis of tert-butyl ((5-(1-(1-(3-(6-(1-(2,2-difluorobenzo[d][1,3]dioxol-5-yl)cyclopropane-1-carboxamido)-3-methylpyridin-2-yl)phenyl)-1,10-dioxo-5,8-dioxa-2,11-diazatridecan-13-yl)-1H-indazol-5-yl)-4-ethyl-3-(4-hydroxyphenyl)pyridin-2-yl)methyl)carbamate Compound 56

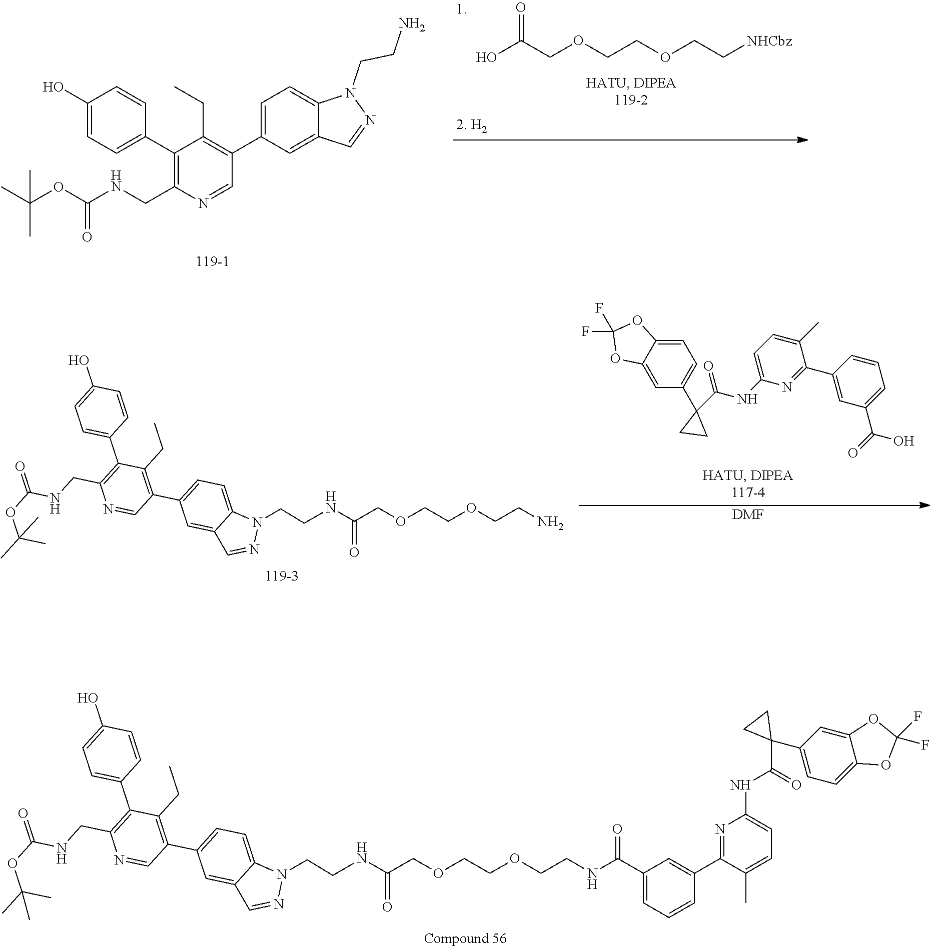

### Example 120. Synthesis of 1-(1-(3-(6-(1-(2,2-difluorobenzo[d][1,3]dioxol-5-yl)cyclopropane-1-carboxamido)-3-methylpyridin-2-yl)phenyl)-1,12-dioxo-5,8-dioxa-2,11-diazatetradecan-14-yl)-N-(6-(thiophene-2-sulfonamido)benzo[d]thiazol-2-yl)piperidine-4-carboxamide Compound 57

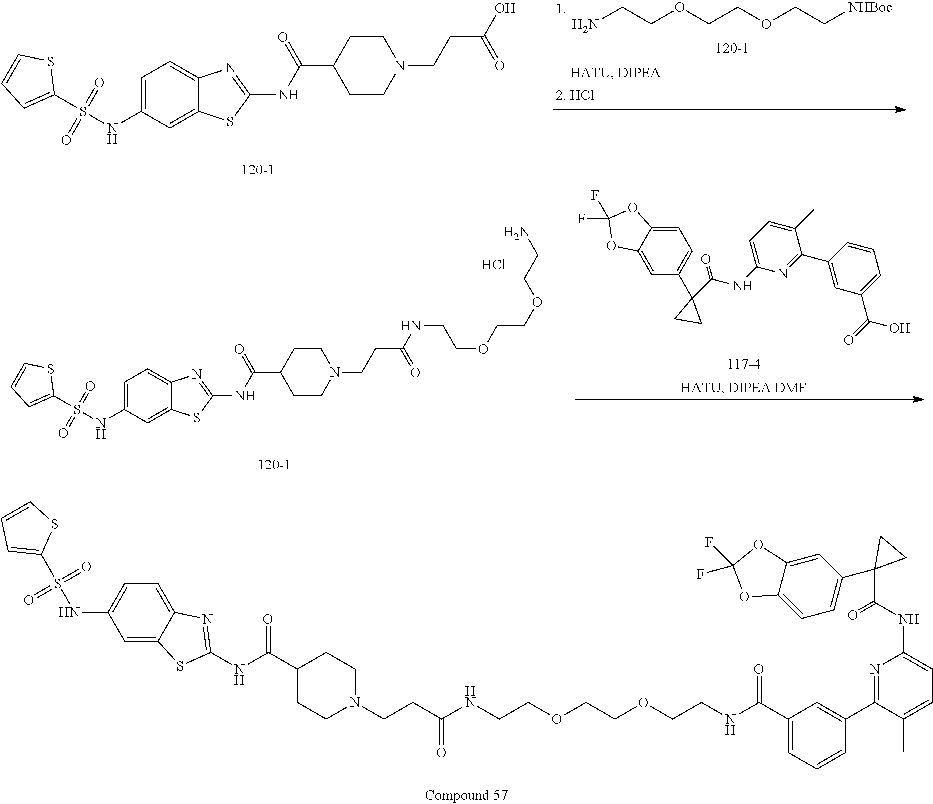 [SMILES: O=C(O)CCN1CCC(C(=O)Nc2nc3ccc(NS(=O)(=O)c4cccs4)cc3s2)CC1 ; CC(C)(C)OC(=O)NCCOCCOCCN ; NCCOCCOCCNC(=O)CCN1CCC(C(=O)Nc2nc3ccc(NS(=O)(=O)c4cccs4)cc3s2)CC1 ; Cl ; Cc1ccc(NC(=O)C2(c3ccc4c(c3)OC(F)(F)O4)CC2)nc1-c1cccc(C(=O)O)c1 ; Cc1ccc(NC(=O)C2(c3ccc4c(c3)OC(F)(F)O4)CC2)nc1-c1cccc(C(=O)NCCOCCOCCNC(=O)CCN2CCC(C(=O)Nc3nc4ccc(NS(=O)(=O)c5cccs5)cc4s3)CC2)c1]

### Example 121. Synthesis of 1-(3-((8-(3-(6-(1-(2,2-difluorobenzo[d][1,3]dioxol-5-yl)cyclopropane-1-carboxamido)-3-methylpyridin-2-yl)benzamido)octyl)amino)-3-oxopropyl)-N-(6-(pyrazine-2-sulfonamido)benzo[d]thiazol-2-yl)piperidine-4-carboxamide Compound 58

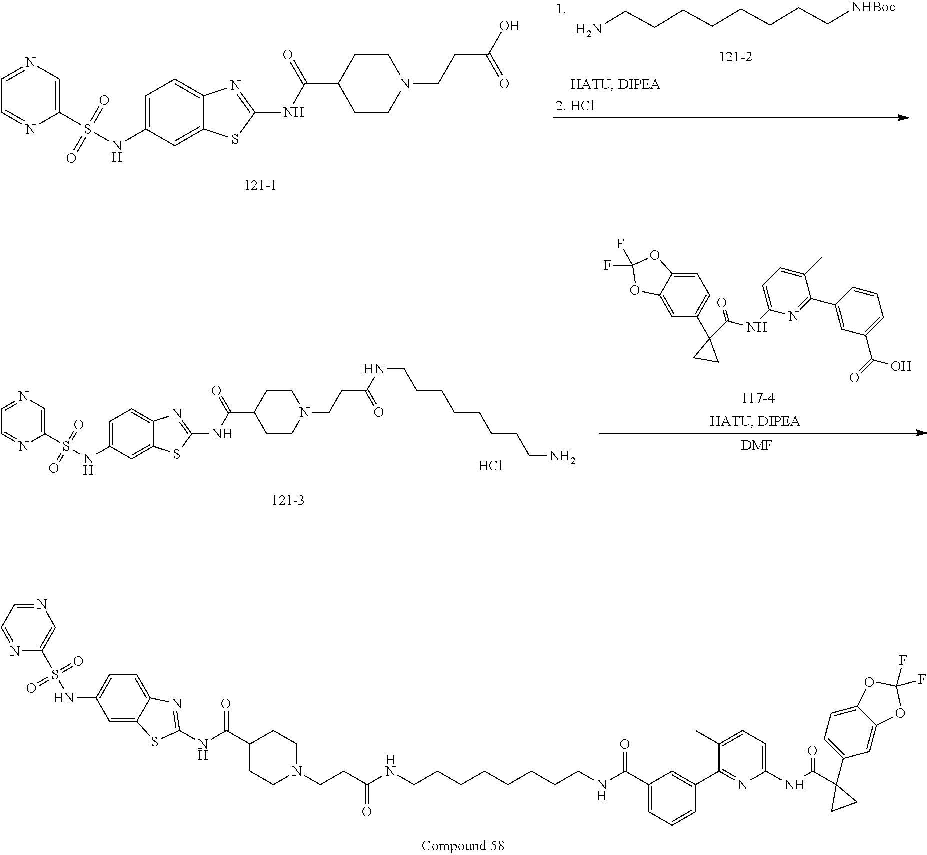

### Example 123. Synthesis of 1-(1-(3-(6-(1-(2,2-difluorobenzo[d][1,3]dioxol-5-yl)cyclopropane-1-carboxamido)-3-methylpyridin-2-yl)phenyl)-1,12-dioxo-5,8-dioxa-2,11-diazatetradecan-14-yl)-N-(6-((3-methylisoxazole)-4-sulfonamido)benzo[d]thiazol-2-yl)piperidine-4-carboxamide Compound 59

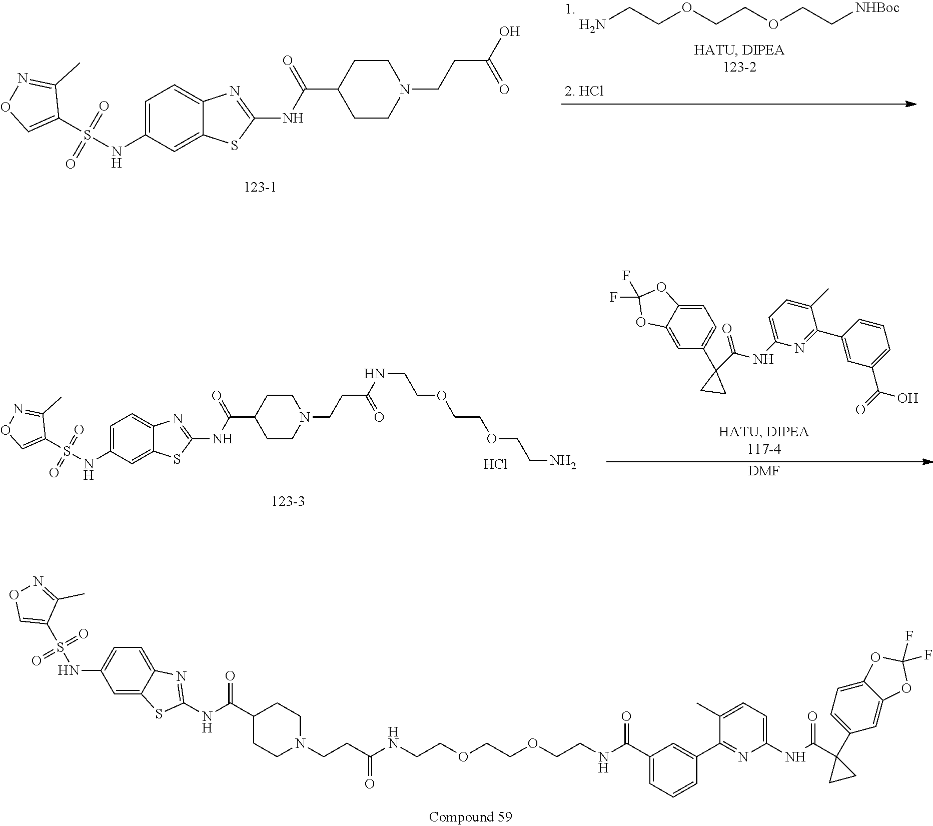

### Example 124. Synthesis of 1-(1-(3-(6-(1-(2,2-difluorobenzo[d][1,3]dioxol-5-yl)cyclopropane-1-carboxamido)-3-methylpyridin-2-yl)phenyl)-1,10-dioxo-5,8-dioxa-2,11-diazatridecan-13-yl)-N-(4-fluoro-6-(thiophene-2-sulfonamido)benzo[d]thiazol-2-yl)piperidine-4-carboxamide Compound 60

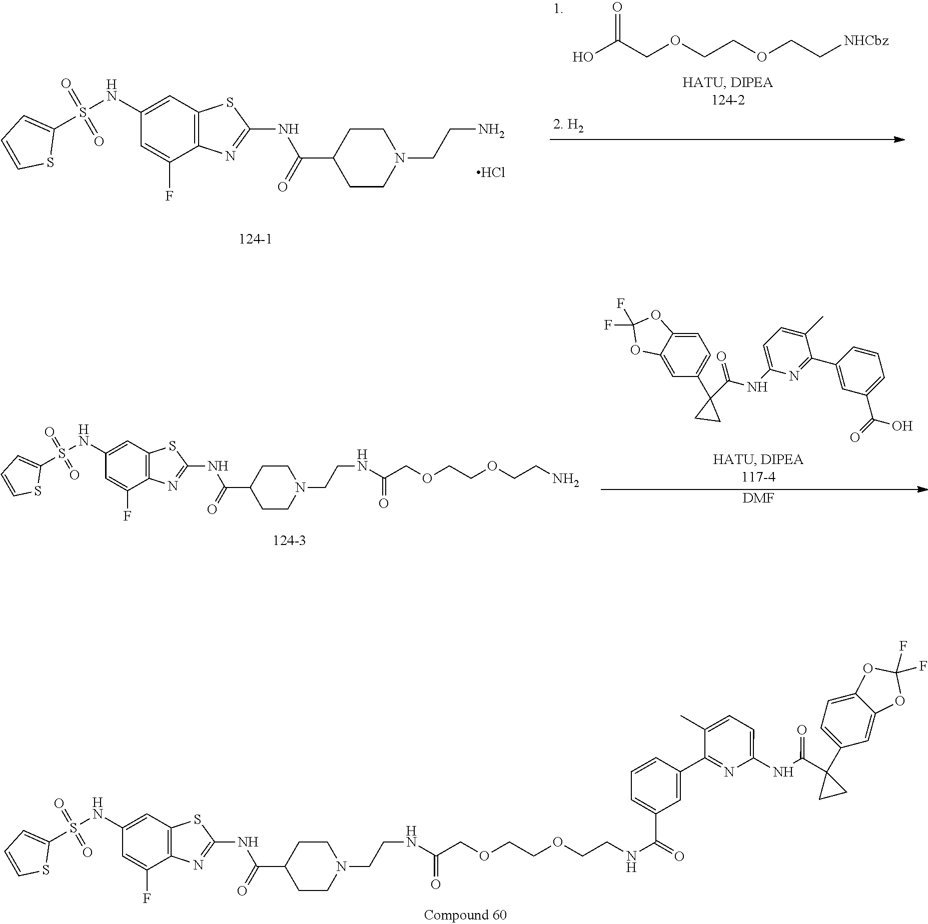

### Example 125. Synthesis of 1-(2-(8-(3-(6-(1-(2,2-difluorobenzo[d][1,3]dioxol-5-yl)cyclopropane-1-carboxamido)-3-methylpyridin-2-yl)benzamido)octanamido)ethyl)-N-(5-fluoro-6-(thiophene-2-sulfonamido)benzo[d]thiazol-2-yl)piperidine-4-carboxamide Compound 61

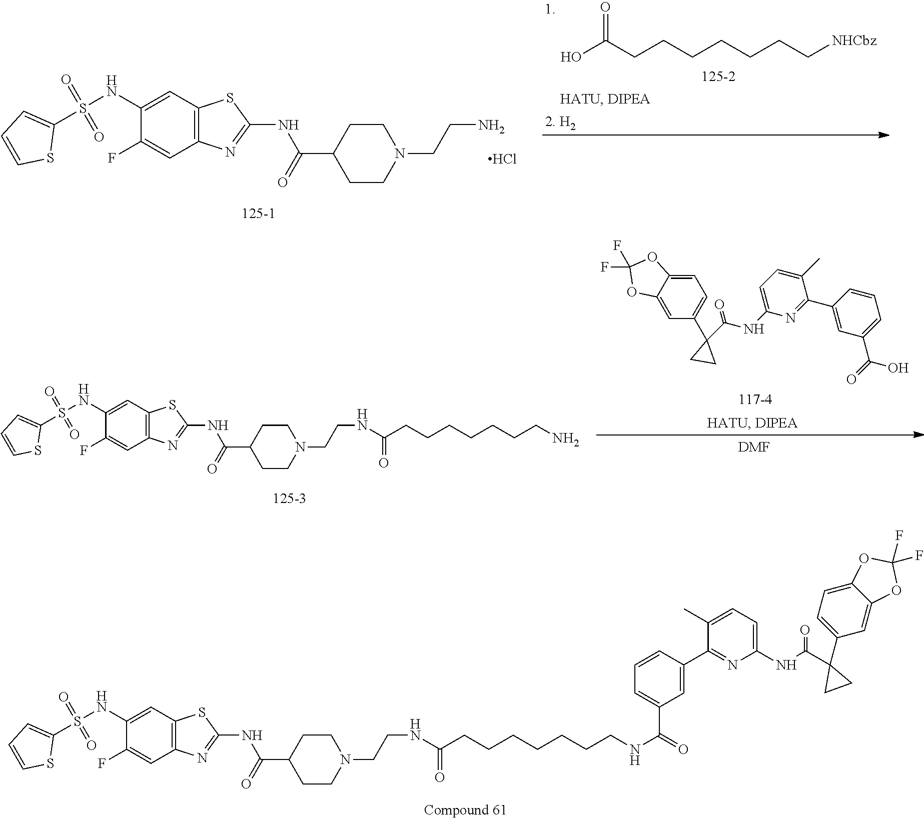

### Example 126. Synthesis of 1-(2-(2-((5-(3-(6-(1-(2,2-difluorobenzo[d][1,3]dioxol-5-yl)cyclopropane-1-carboxamido)-3-methylpyridin-2-yl)benzamido)pentyl)oxy)acetamido)ethyl)-N-(7-fluoro-6-(thiophene-2-sulfonamido)benzo[d]thiazol-2-yl)piperidine-4-carboxamide Compound 62

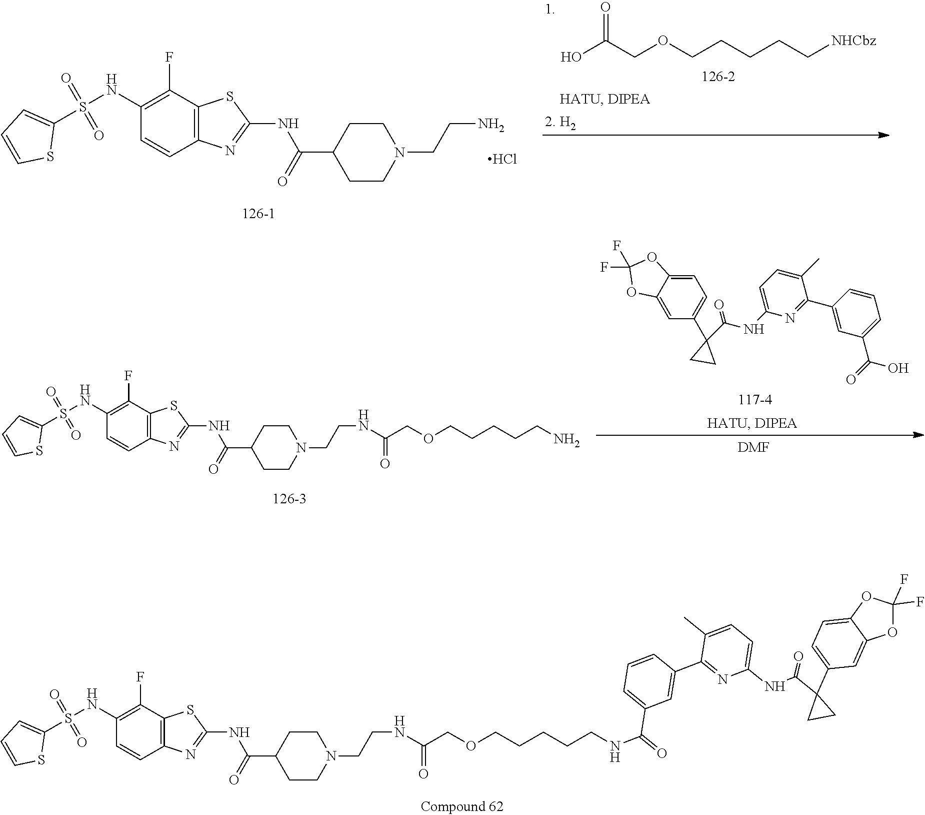

### Example 127. Synthesis of N-(1-(2-(1-(3-(6-(1-(2,2-difluorobenzo[d][1,3]dioxol-5-yl)cyclopropane-1-carboxamido)-3-methylpyridin-2-yl)benzoyl)piperidin-4-yl)ethyl)piperidin-4-yl)-6-(thiophene-2-sulfonamido)benzo[d]thiazole-2-carboxamide Compound 63

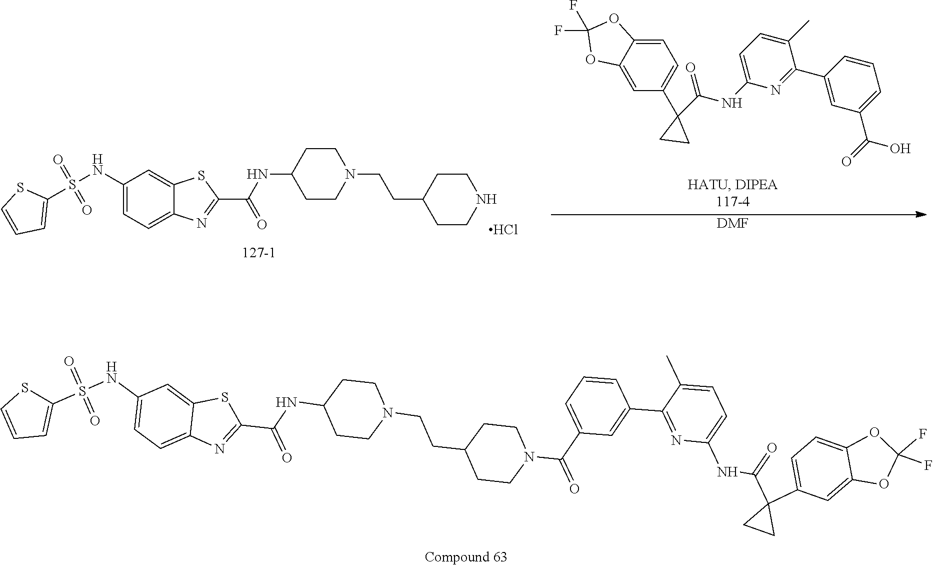

### Example 128. Synthesis of 1-(2-(1-(2-(3-(6-(1-(2,2-difluorobenzo[d][1,3]dioxol-5-yl)cyclopropane-1-carboxamido)-3-methylpyridin-2-yl)benzamido)ethyl)-1H-pyrazol-4-yl)ethyl)-N-(6-(thiophene-2-sulfonamido)thiazolo[4,5-c]pyridin-2-yl)piperidine-4-carboxamide Compound 64

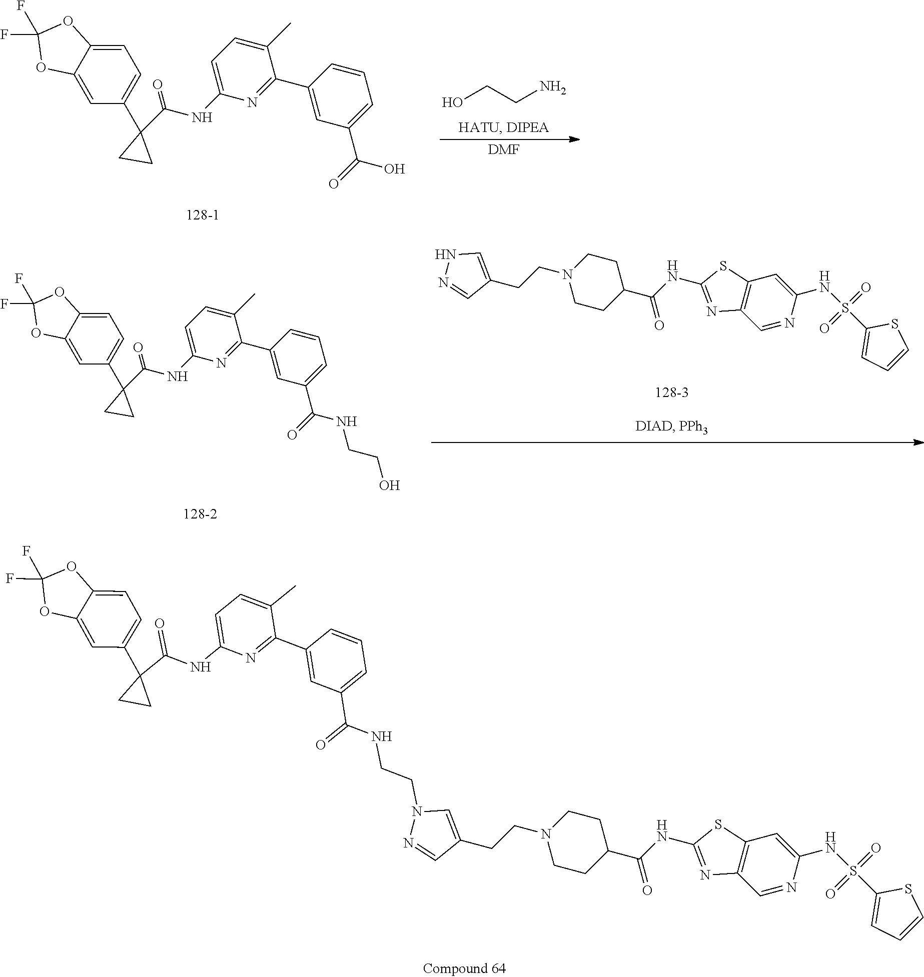

### Example 129. Synthesis of 1-(1-(3-(6-(1-(2,2-difluorobenzo[d][1,3]dioxol-5-yl)cyclopropane-1-carboxamido)-3-methylpyridin-2-yl)phenyl)-1,12-dioxo-5,8-dioxa-2,11-diazatetradecan-14-yl)-N-(6-(pyridine-4-sulfonamido)benzo[d]oxazol-2-yl)piperidine-4-carboxamide Compound 65

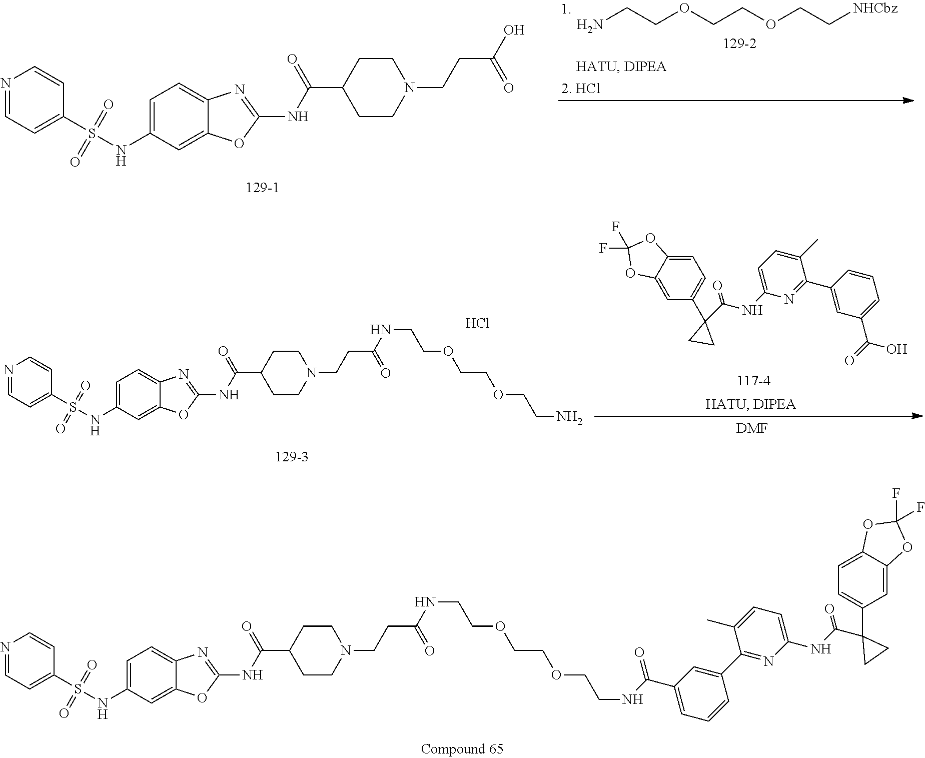

### Example 130. Synthesis of 1-(1-(3-(6-(1-(2,2-difluorobenzo[d][1,3]dioxol-5-yl)cyclopropane-1-carboxamido)-3-methylpyridin-2-yl)phenyl)-1,12-dioxo-5,8-dioxa-2,11-diazatetradecan-14-yl)-N-(5-(pyridine-2-sulfonamido)benzo[d]thiazol-2-yl)piperidine-4-carboxamide Compound 66

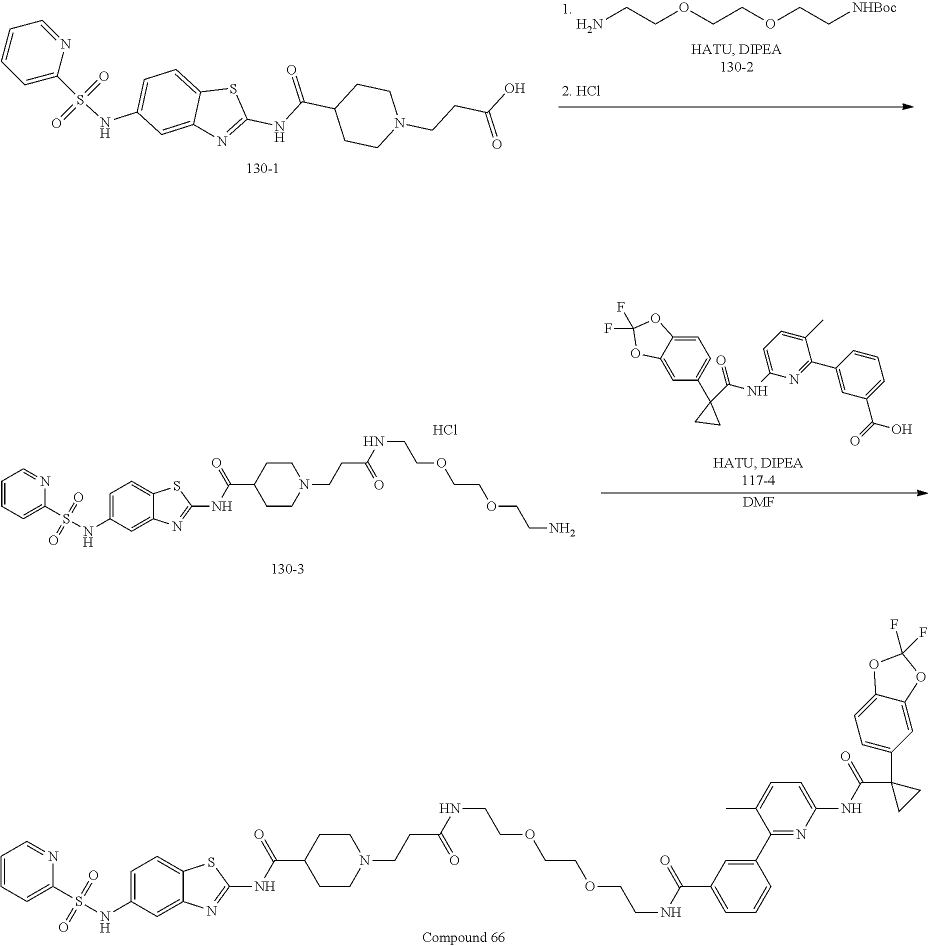

### Example 131. Synthesis of 1-(2-(1-(2-(2-(3-(6-(1-(2,2-difluorobenzo[d][1,3]dioxol-5-yl)cyclopropane-1-carboxamido)-3-methylpyridin-2-yl)benzamido)ethoxy)ethyl)-1H-pyrazol-4-yl)ethyl)-N-(6-(thiophene-2-sulfonamido)thiazolo[4,5-b]pyridin-2-yl)piperidine-4-carboxamide Compound 67

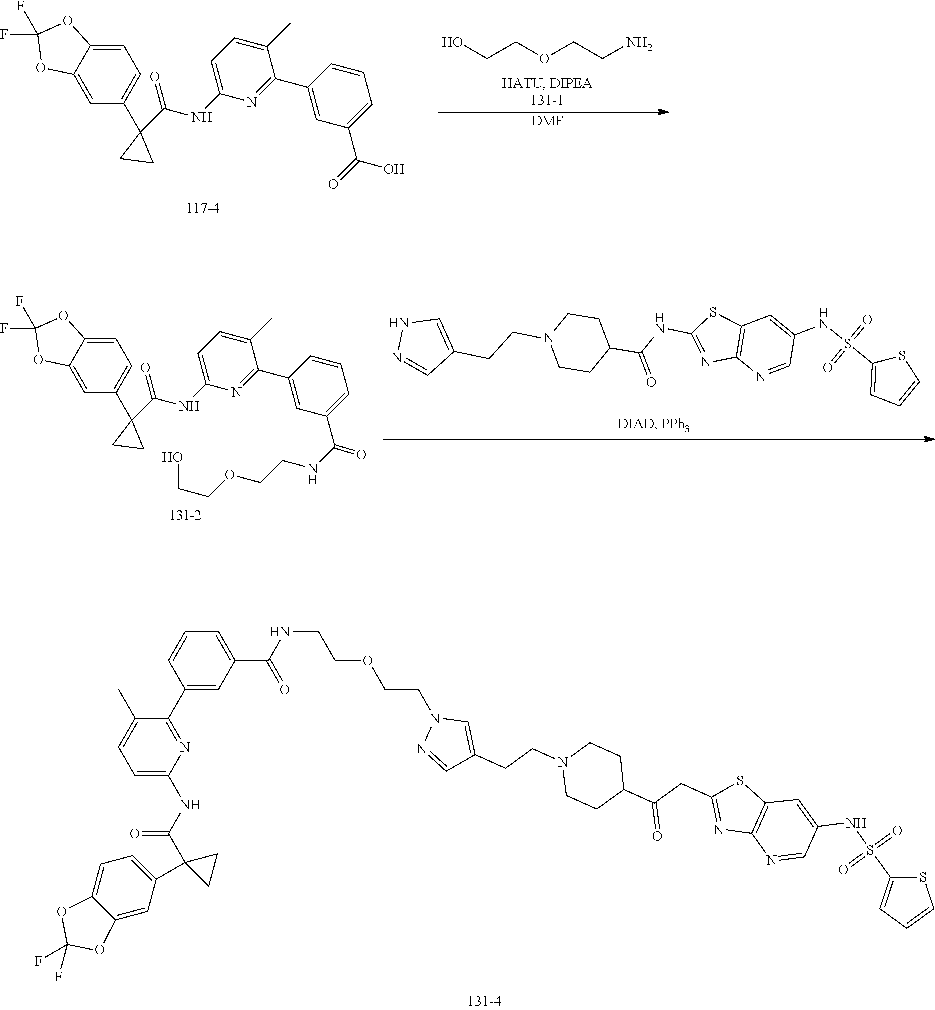

### Example 132. Synthesis of 1-(2-(1-(2-(3-(6-(1-(2,2-difluorobenzo[d][1,3]dioxol-5-yl)cyclopropane-1-carboxamido)-3-methylpyridin-2-yl)benzamido)ethyl)-1H-pyrazol-4-yl)ethyl)-N-(5-(thiophene-2-sulfonamido)thiazolo[5,4-b]pyridin-2-yl)piperidine-4-carboxamide Compound 68

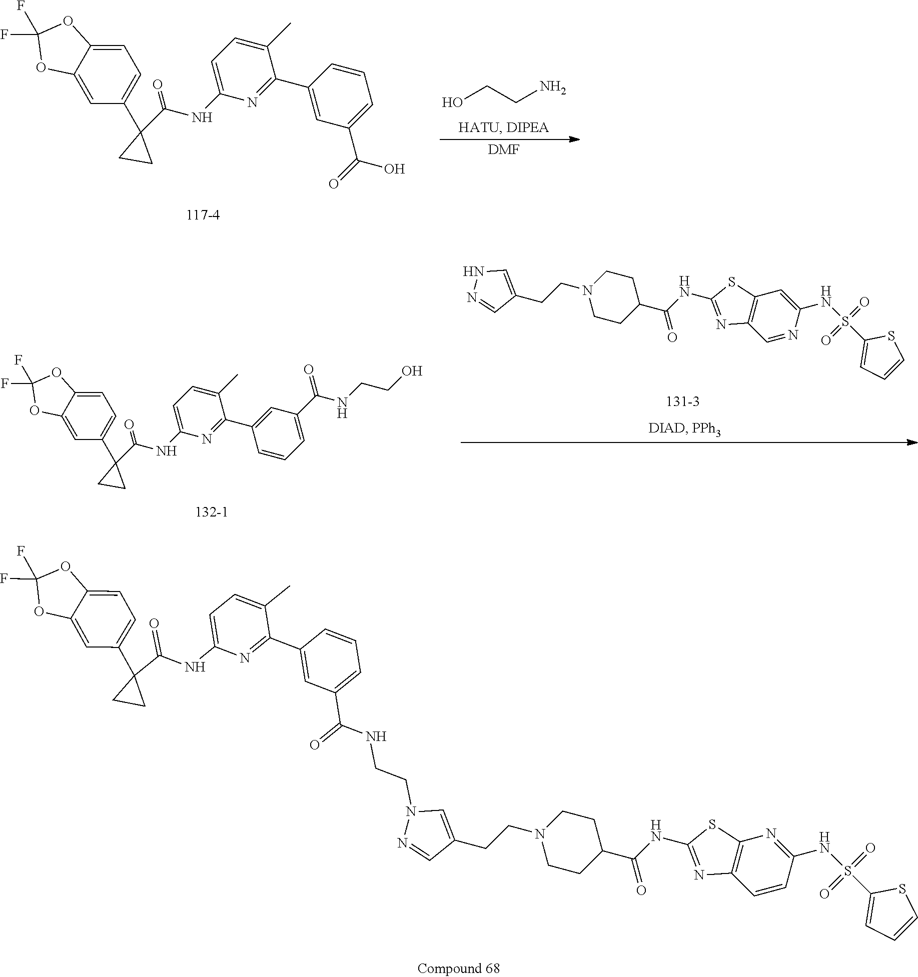

### Example 133. Synthesis of 1-(1-(3-(6-(1-(2,2-difluorobenzo[d][1,3]dioxol-5-yl)cyclopropane-1-carboxamido)-3-methylpyridin-2-yl)phenyl)-1,10-dioxo-5,8-dioxa-2,11-diazatridecan-13-yl)-N-(6-(N-(2-(dimethylamino)ethyl)thiophene-2-sulfonamido)-4-fluorobenzo[d]thiazol-2-yl)piperidine-4-carboxamide Compound 69

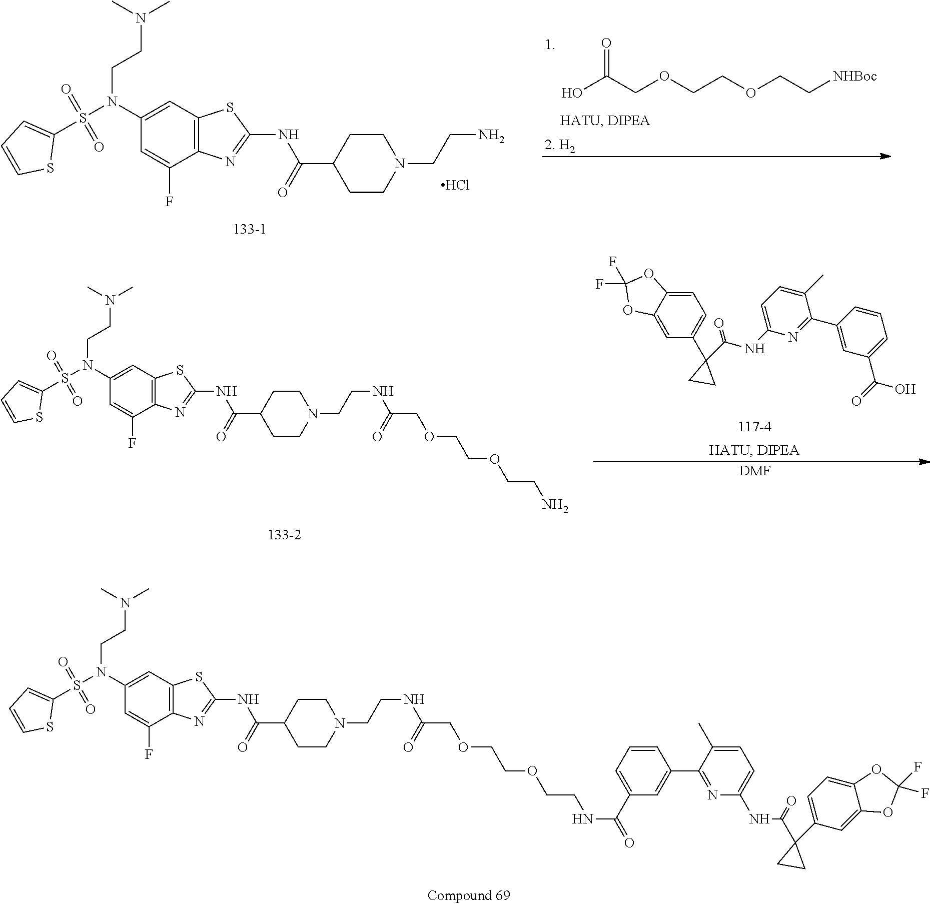

### Example 134. Synthesis of 1-(1-(3-(6-(1-(2,2-difluorobenzo[d][1,3]dioxol-5-yl)cyclopropane-1-carboxamido)-3-methylpyridin-2-yl)phenyl)-1,10-dioxo-5,8-dioxa-2,11-diazatridecan-13-yl)-N-(6-(N-(2-(dimethylamino)ethyl)thiophene-2-sulfonamido)-4-fluorobenzo[d]thiazol-2-yl)piperidine-4-carboxamide Compound 70

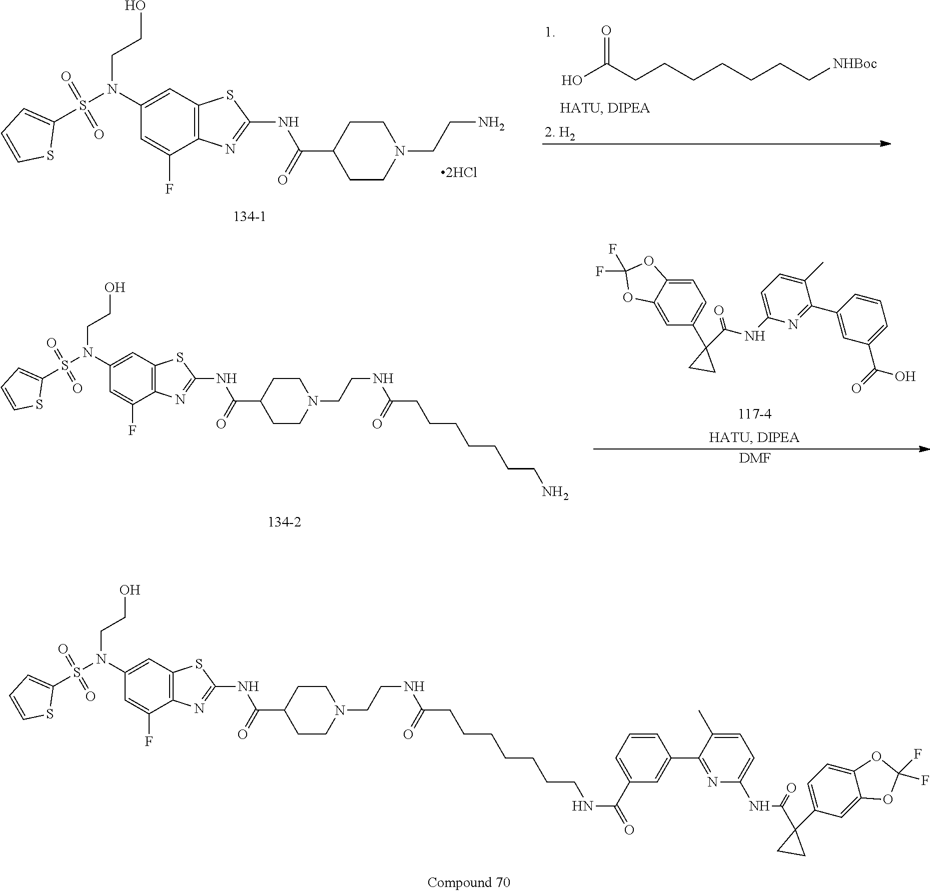

### Example 135. Synthesis of 1-(2-(8-(3-(6-(1-(2,2-difluorobenzo[d][1,3]dioxol-5-yl)cyclopropane-1-carboxamido)-3-methylpyridin-2-yl)benzamido)octanamido)ethyl)-N-(5-fluoro-6-(thiophene-2-sulfonamido)benzo[d]thiazol-2-yl)piperidine-4-carboxamide Compound 71

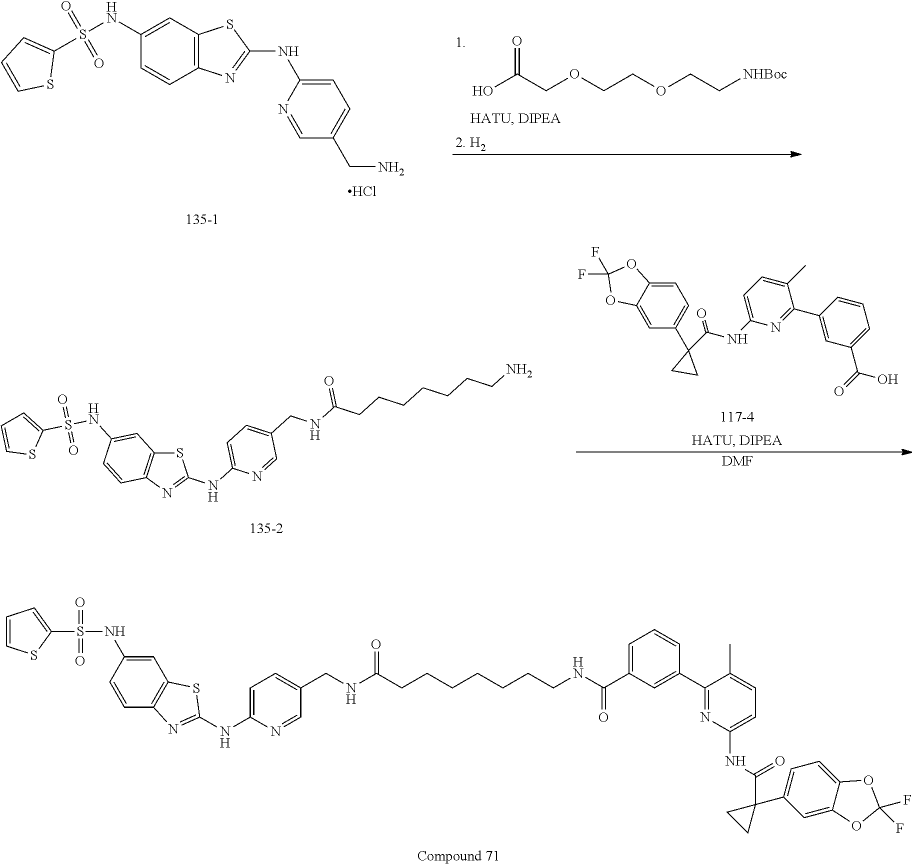

### Example 136. Synthesis of 5-((1-(2-(2-(2-(3-(6-(1-(2,2-difluorobenzo[d][1,3]dioxol-5-yl)cyclopropane-1-carboxamido)-3-methylpyridin-2-yl)benzamido)ethoxy)ethoxy)acetyl)piperidin-4-yl)methyl)-N-(6-(thiophene-2-sulfonamido)benzo[d]thiazol-2-yl)-5,6,7,8-tetrahydro-4H-pyrazolo[1,5-a][1,4]diazepine-2-carboxamide Compound 72

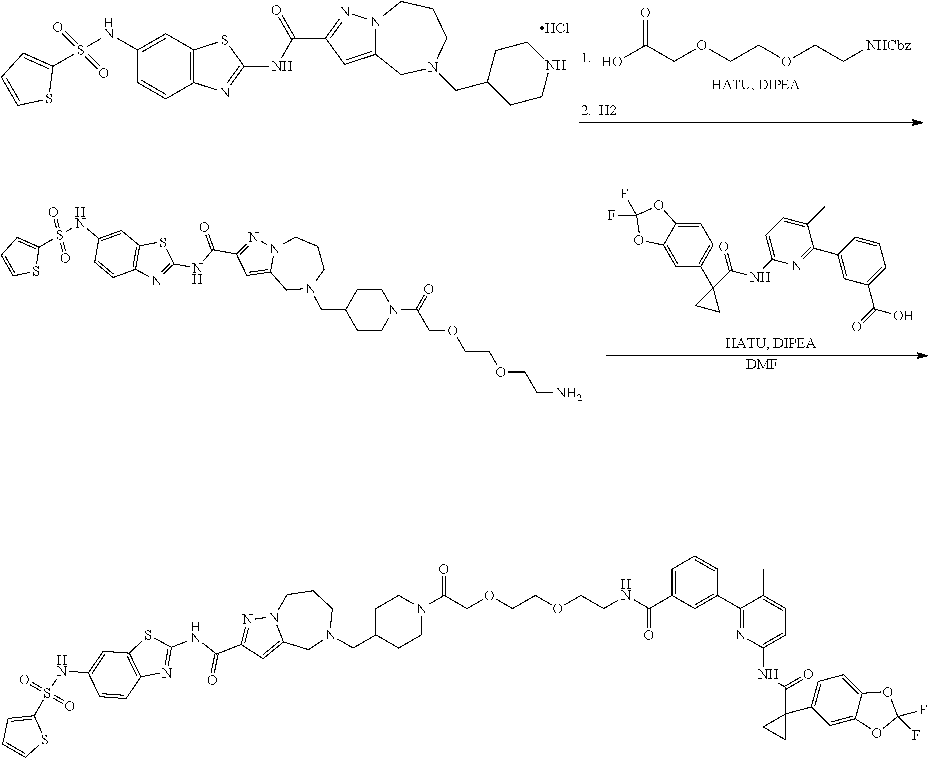

### Example 137. Synthesis of (E)-4-(2-(4-(2-(2-ethoxyphenyl)hydrazineylidene)-3-methyl-5-oxo-4,5-dihydro-1H-pyrazol-1-yl)thiazol-4-yl)benzoic acid

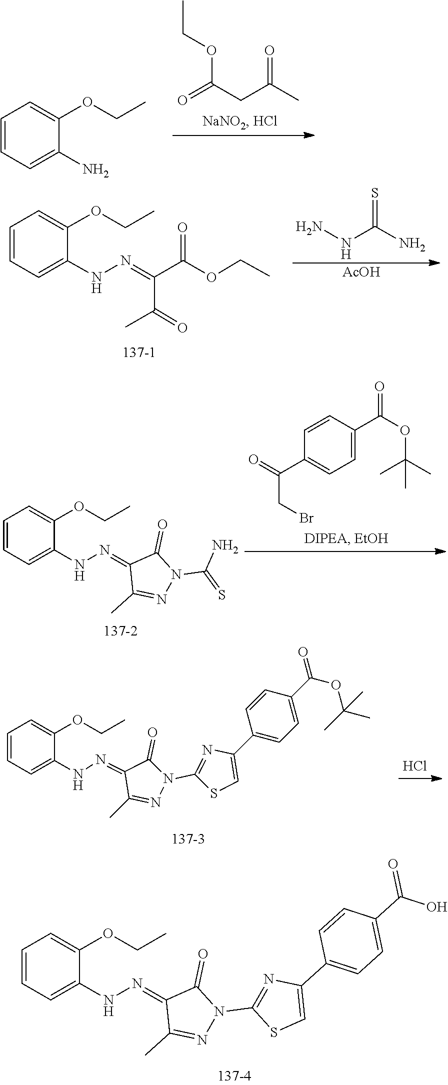

### Example 138. Synthesis of (E)-3′-(4-(2-(2-ethoxyphenyl)hydrazineylidene)-3-methyl-5-oxo-4,5-dihydro-1H-pyrazol-1-yl)-5′-hydroxy-[1,1′-biphenyl]-4-carboxylic acid

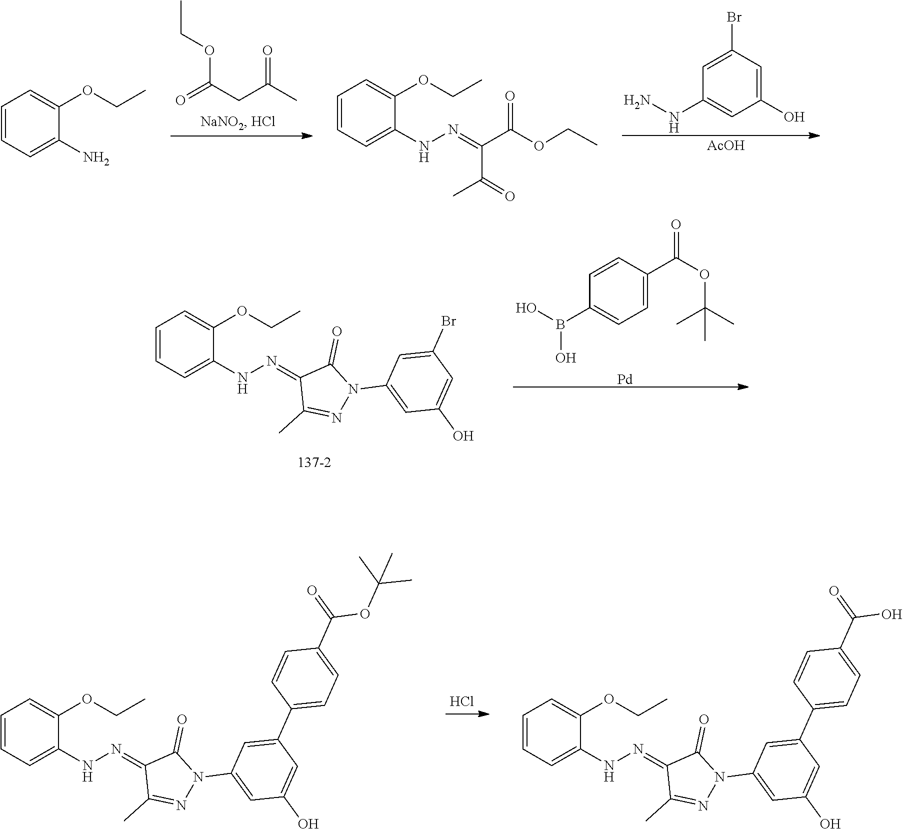

### Example 139. Synthesis of (S)-1-(4-((4-azido-2,3,5,6-tetramethylphenyl)sulfonamido)naphthalen-1-yl)pyrrolidine-3-carboxylic acid

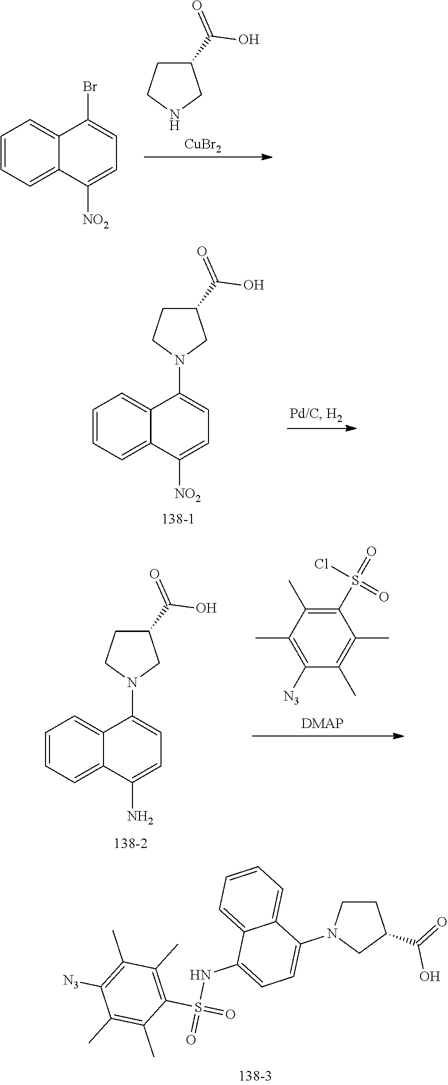

### Example 140. Synthesis of benzyl 3-(((trifluoromethyl)sulfonyl)oxy)cyclohex-2-ene-1-carboxylate

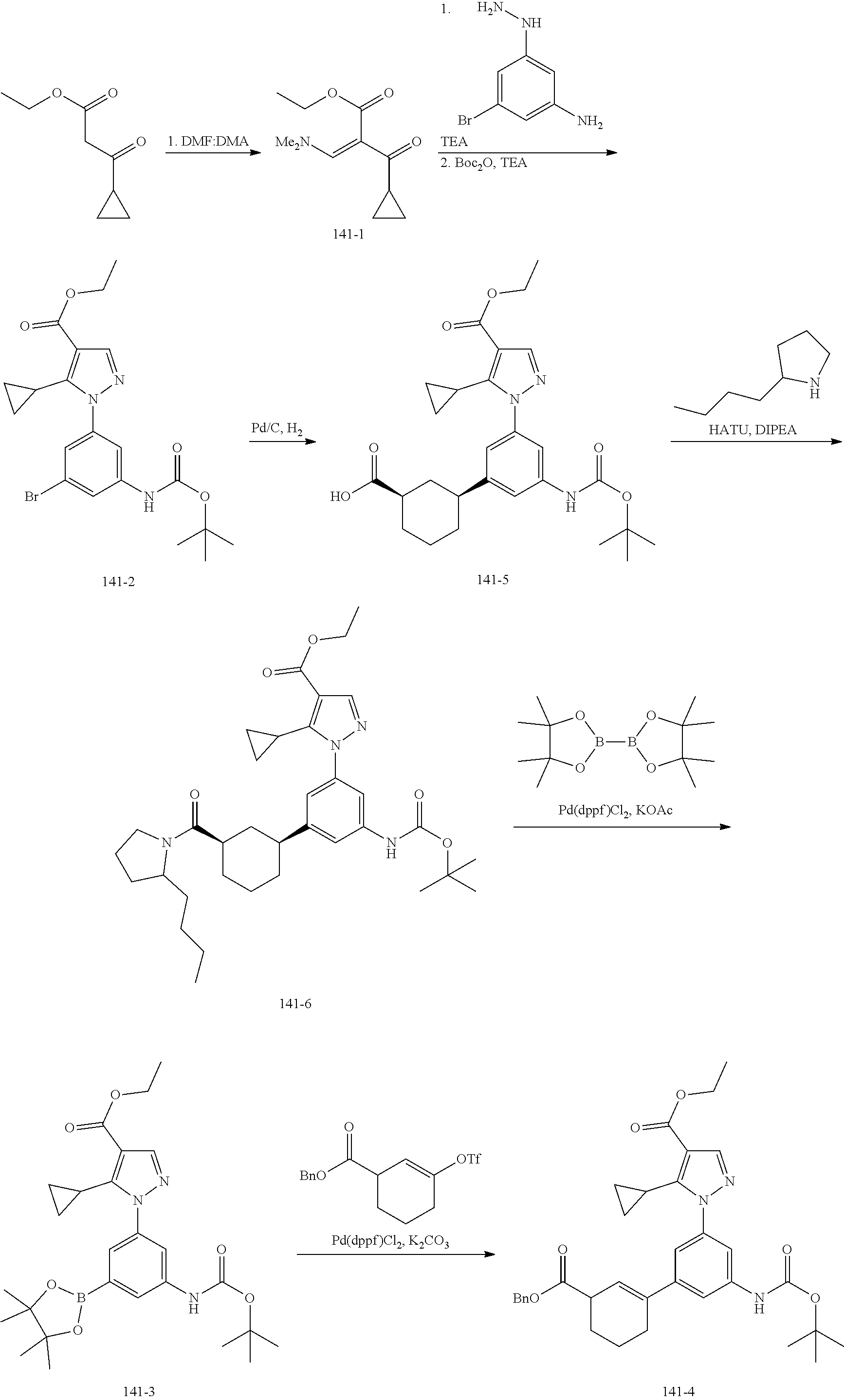

### Example 141. Synthesis of 1-(3-amino-5-((1S,3R)-3-(2-butylpyrrolidine-1-carbonyl)cyclohexyl)phenyl)-5-cyclopropyl-1H-pyrazole-4-carboxylic acid Compound 51

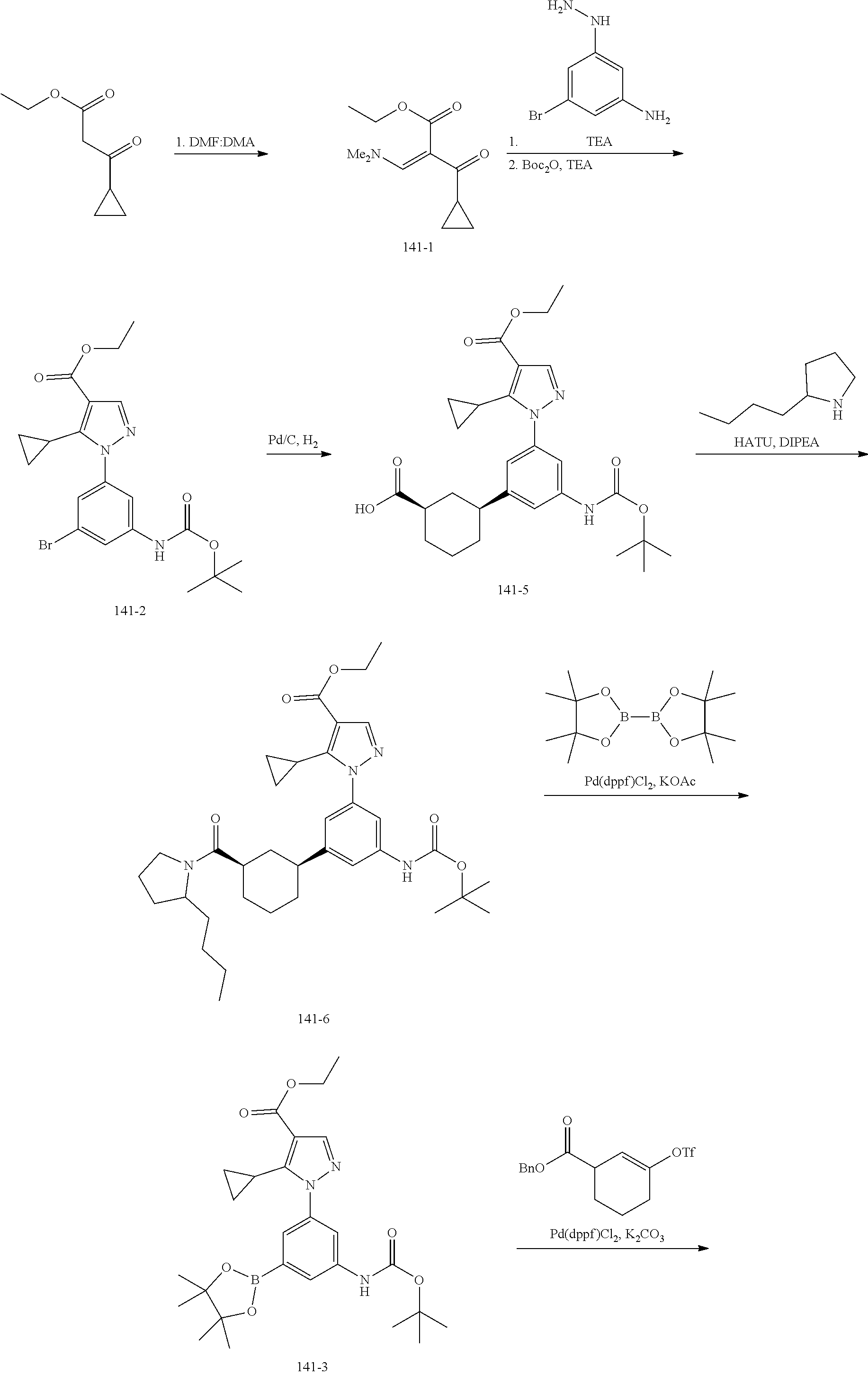

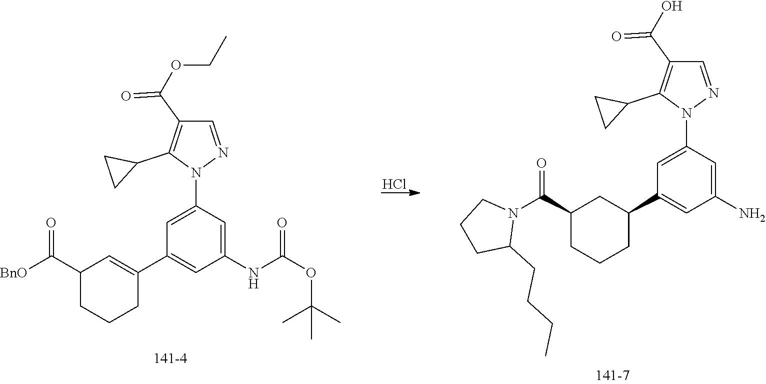 [SMILES: CCOC(=O)c1cnn(-c2cc(NC(=O)OC(C)(C)C)cc(C3=CC(C(=O)OCc4ccccc4)CCC3)c2)c1C1CC1 ; Cl ; CCCCC1CCCN1C(=O)[C@@H]1CCC[C@H](c2cc(N)cc(-n3ncc(C(=O)O)c3C3CC3)c2)C1]

### Example 142. Synthesis of 1-((4-aminophenyl)sulfonyl)-5-((4-ethoxyphenyl)sulfonamido)-N-hydroxy-2-methyl-1H-benzo[g]indole-3-carboxamide

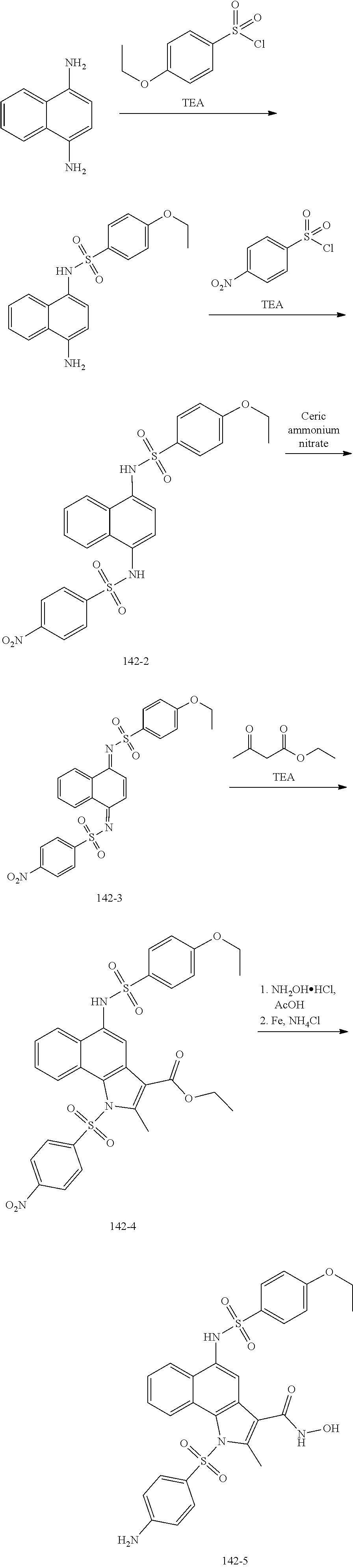

### Example 143. Synthesis of N-(3-(aminomethyl)isothiazol-5-yl)-4-(((1r,4r)-4-morpholinocyclohexyl)oxy)furo[3,2-d]pyrimidin-2-amine hydrochloride

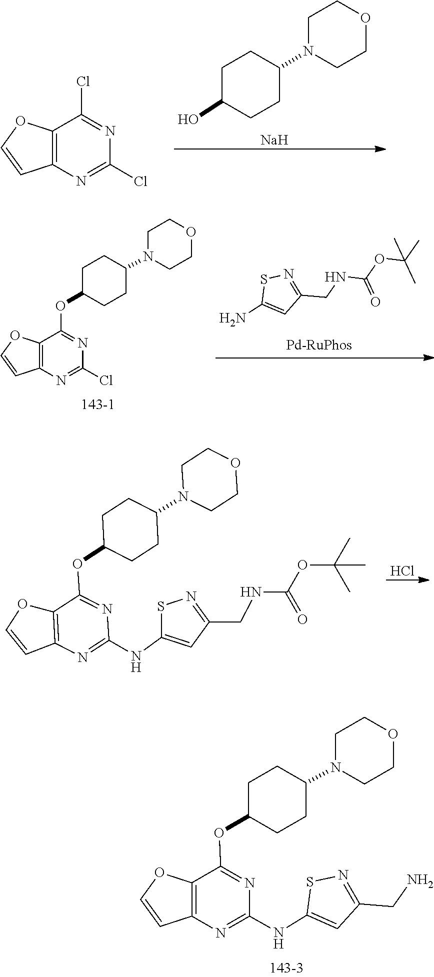

### Example 144. Synthesis of 4-(((1r,4r)-4-morpholinocyclohexyl)oxy)-N-(1-(piperidin-4-yl)-1H-pyrazol-4-yl)furo[3,2-d]pyrimidin-2-amine hydrochloride

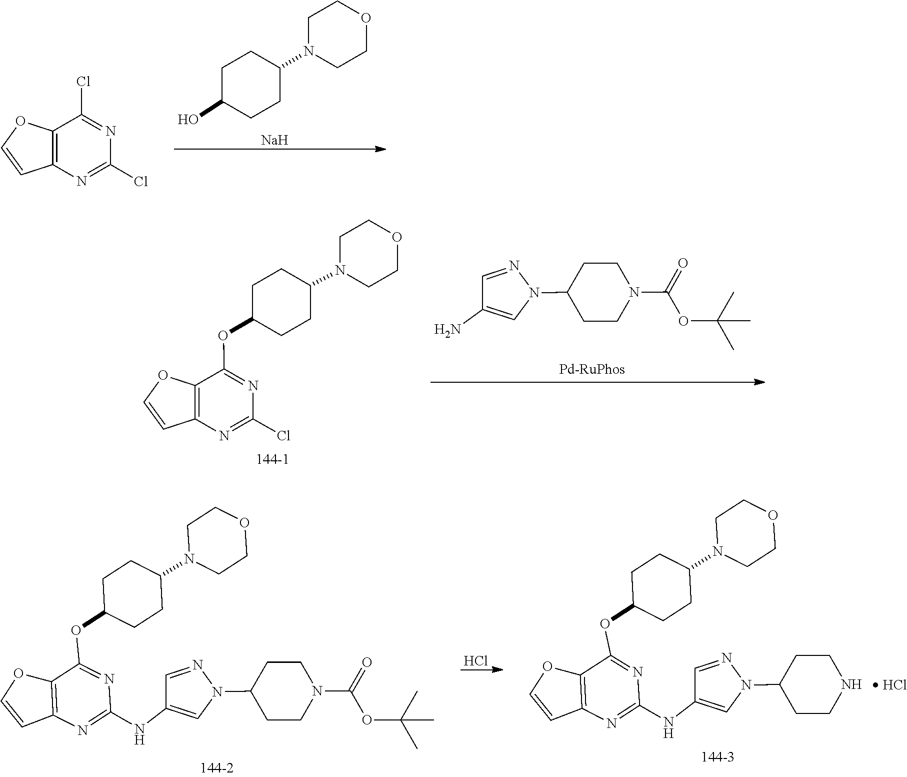

### Example 145 Synthesis of (E)-4-((4-aminobut-2-en-1-yl)amino)-3-methoxy-5-nitrobenzamide hydrochloride

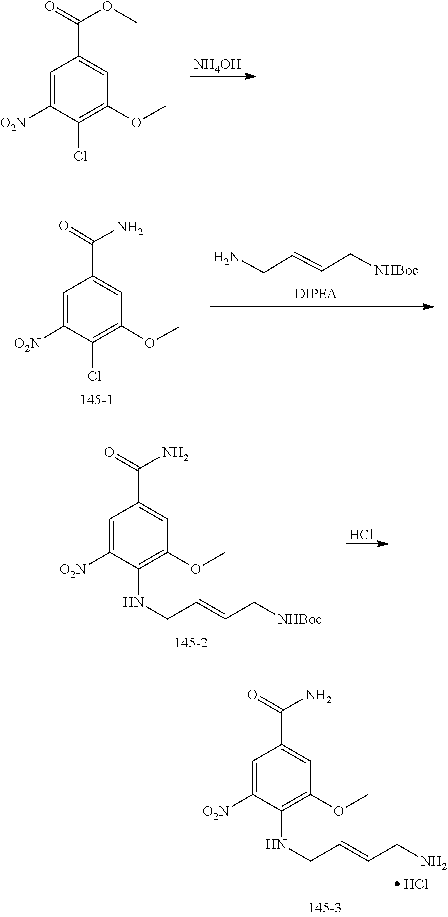

### Example 146 (Z)-1-((E)-4-((Z)-5-carbamoyl-2-((1-ethyl-3-methyl-1H-pyrazole-5-carbonyl)imino)-7-(3-(piperazin-1-yl)propoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide trifluoroacetate

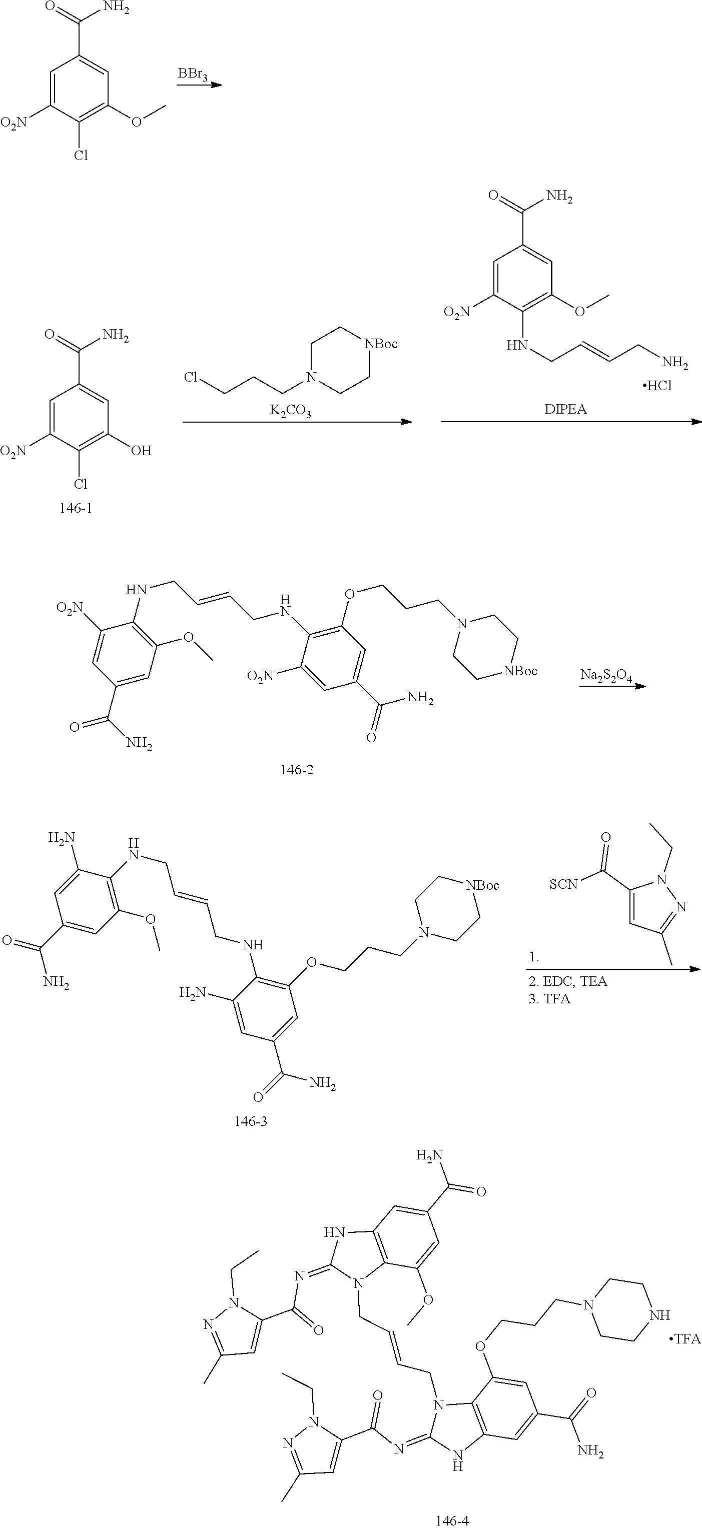

### Example 147. N-(6-amino-9,10-dioxo-9,10-dihydrophenanthren-2-yl)pivalamide

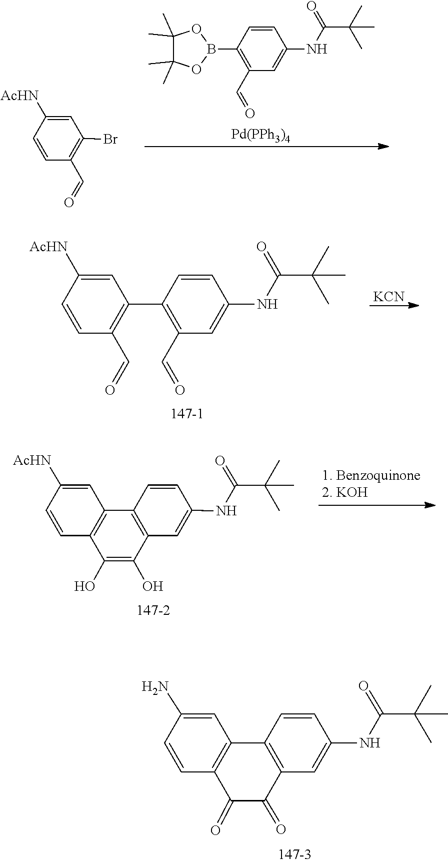

### Example 148. 1-((2R,4S,5S)-4-azido-5-(hydroxymethyl)tetrahydrofuran-2-yl)-5-((E)-2-bromovinyl)pyrimidine-2,4(1H,3H)-dione

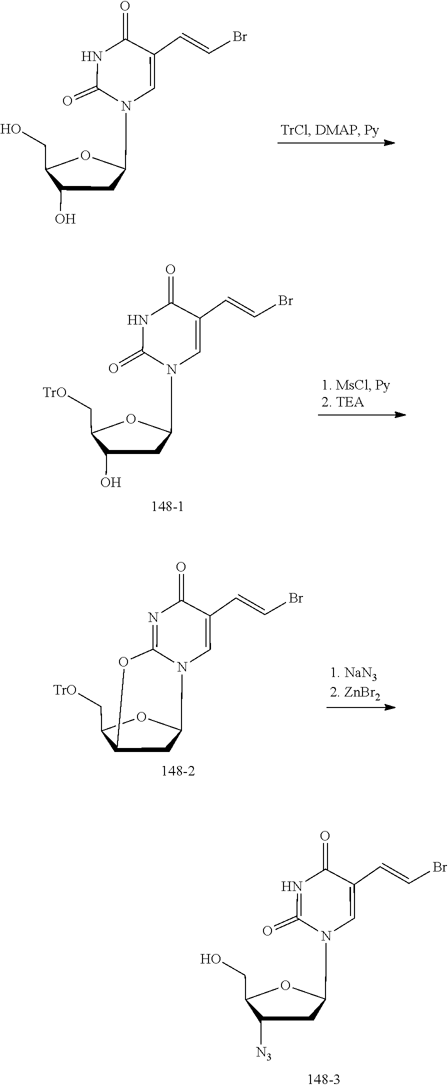

### Example 149. (R)-1-((4-(aminomethyl)phenyl)sulfonyl)-N-(4-(4-methoxyphenyl)thiazol-2-yl)piperidine-2-carboxamide

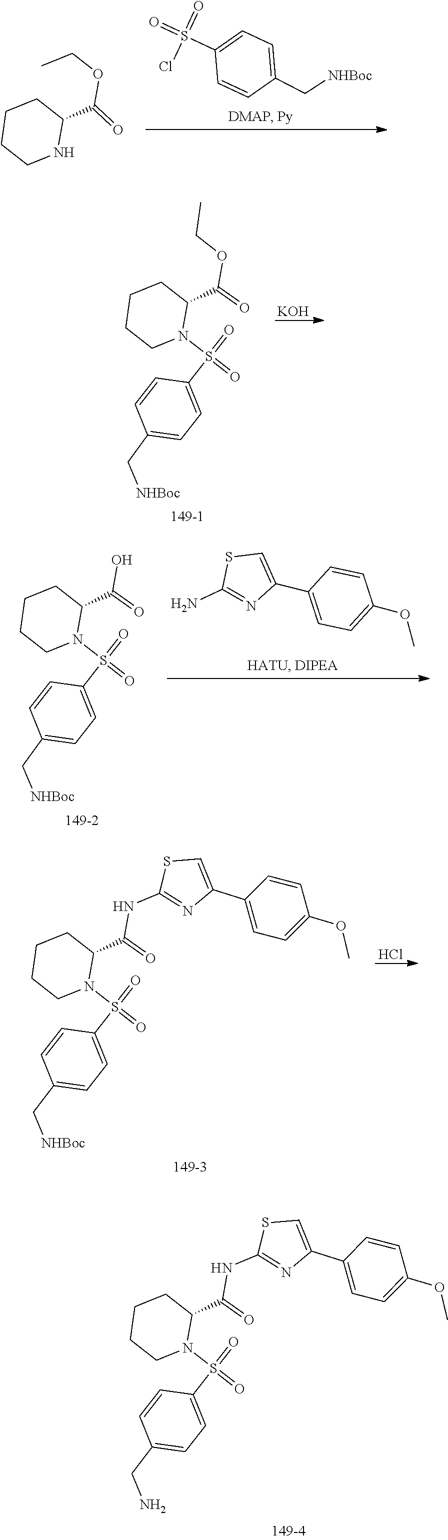

### Example 150. Synthesis of (E)-5-((1-(2-(2-(2-(4-(2-(4-(2-(2-ethoxyphenyl)hydrazineylidene)-3-methyl-5-oxo-4,5-dihydro-1H-pyrazol-1-yl)thiazol-4-yl)benzamido)ethoxy)ethoxy)acetyl)piperidin-4-yl)methyl)-N-(6-(thiophene-2-sulfonamido)benzo[d]thiazol-2-yl)-5,6,7,8-tetrahydro-4H-pyrazolo[1,5-a][1,4]diazepine-2-carboxamide Compound 73

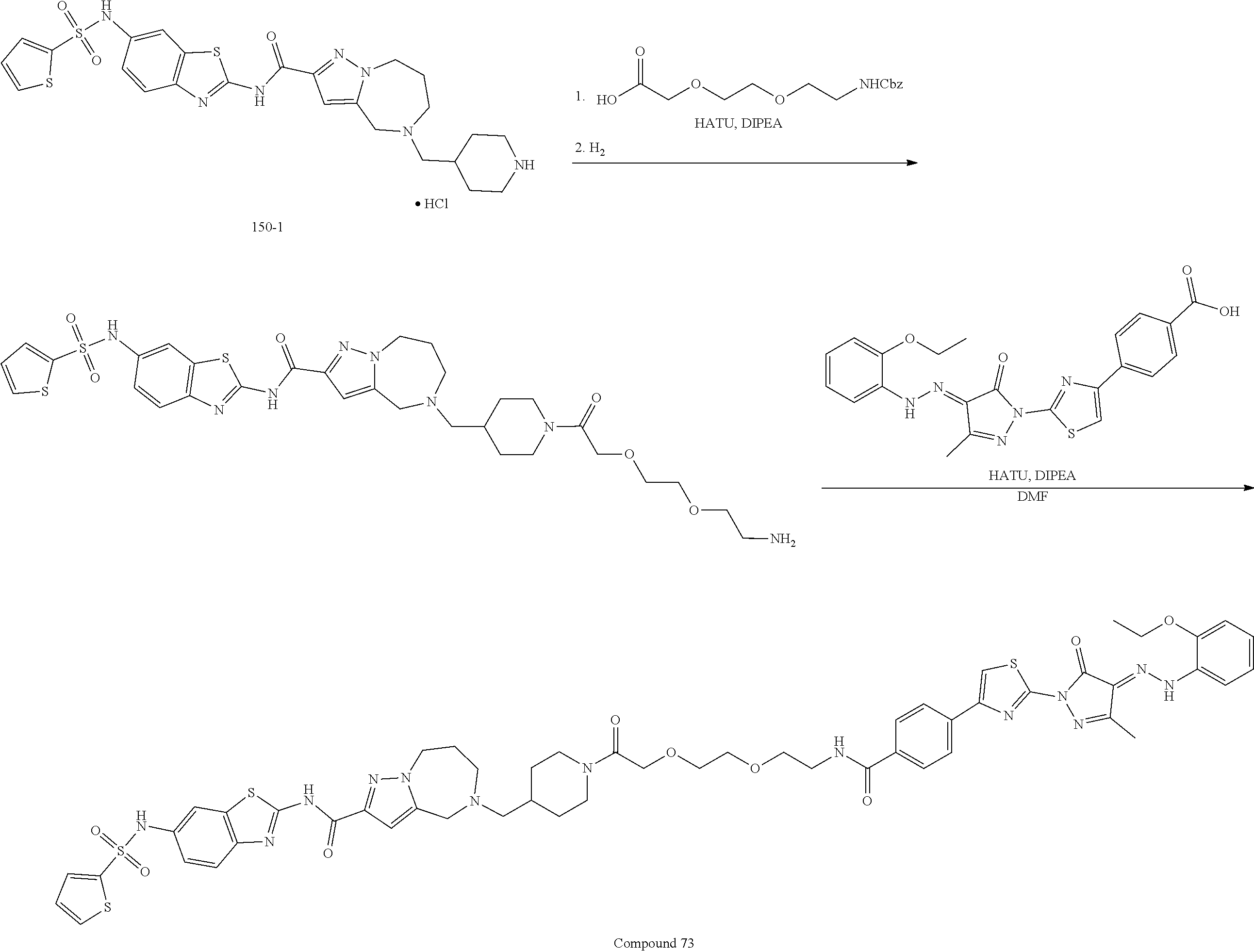

### Example 151. Synthesis of (E)-3′-(4-(2-(2-ethoxyphenyl)hydrazineylidene)-3-methyl-5-oxo-4,5-dihydro-1H-pyrazol-1-yl)-5′-hydroxy-N-(8-oxo-8-(((6-((6-(thiophene-2-sulfonamido)benzo[d]thiazol-2-yl)amino)pyridin-3-yl)methyl)amino)octyl)-[1,1′-biphenyl]-4-carboxamide Compound 74

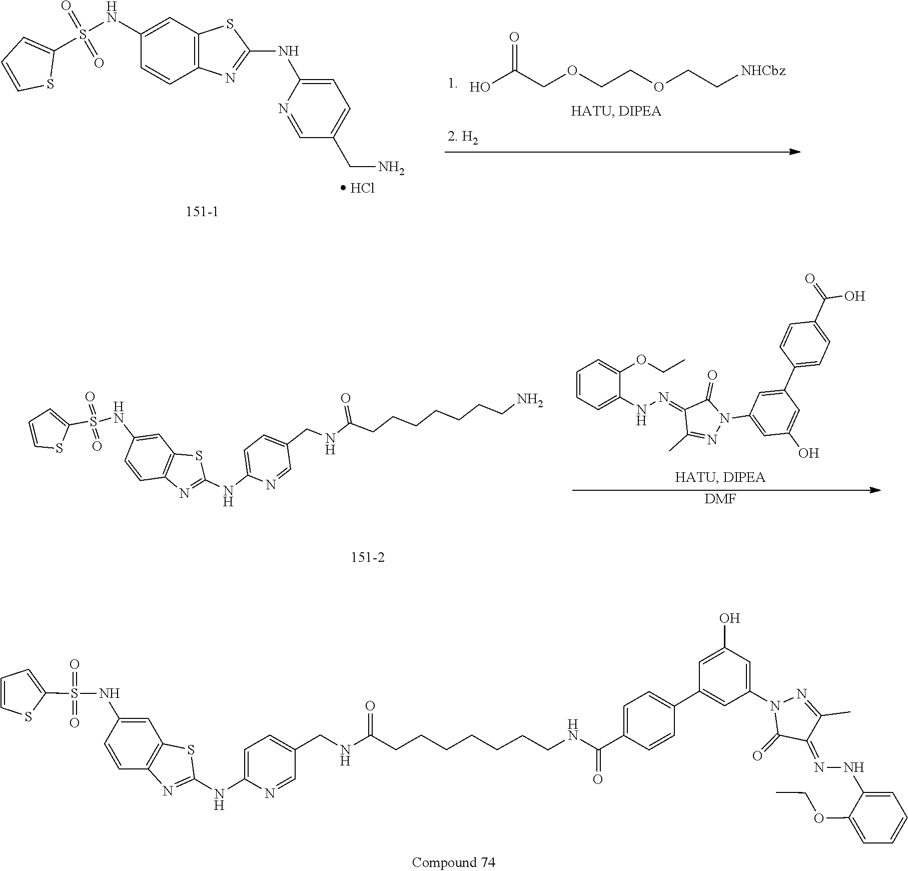

### Example 152. Synthesis of (S)-1-(4-((4-(4-(7-((2-(4-((4-fluoro-6-(N-(2-hydroxyethyl)thiophene-2-sulfonamido)benzo[d]thiazol-2-yl)carbamoyl)piperidin-1-yl)ethyl)amino)-7-oxoheptyl)-1H-1,2,3-triazol-1-yl)-2,3,5,6-tetramethylphenyl)sulfonamido)naphthalen-1-yl)pyrrolidine-3-carboxylic acid Compound

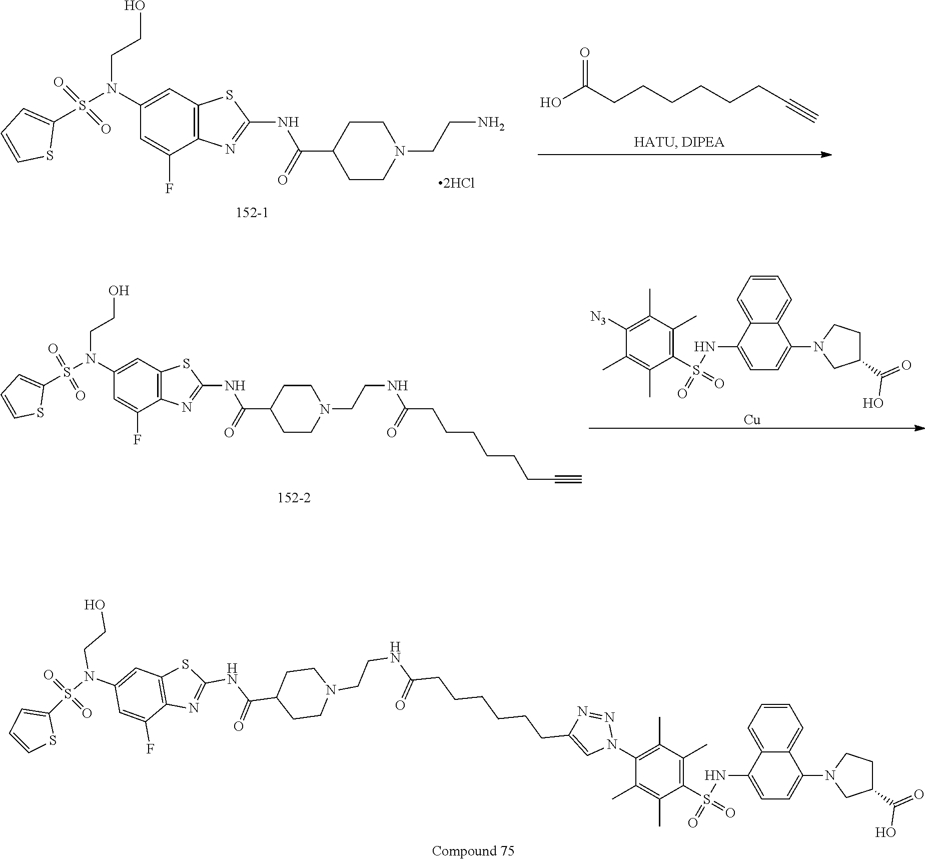

### Example 153. Synthesis of 1-(3-((1S,3R)-3-(2-butylpyrrolidine-1-carbonyl)cyclohexyl)-5-((2-(2-(2-((2-(4-((6-(N-(2-(dimethylamino)ethyl)thiophene-2-sulfonamido)-4-fluorobenzo[d]thiazol-2-yl)carbamoyl)piperidin-1-yl)ethyl)amino)-2-oxoethoxy)ethoxy)ethyl)amino)phenyl)-5-cyclopropyl-1H-pyrazole-4-carboxylic acid Compound 76

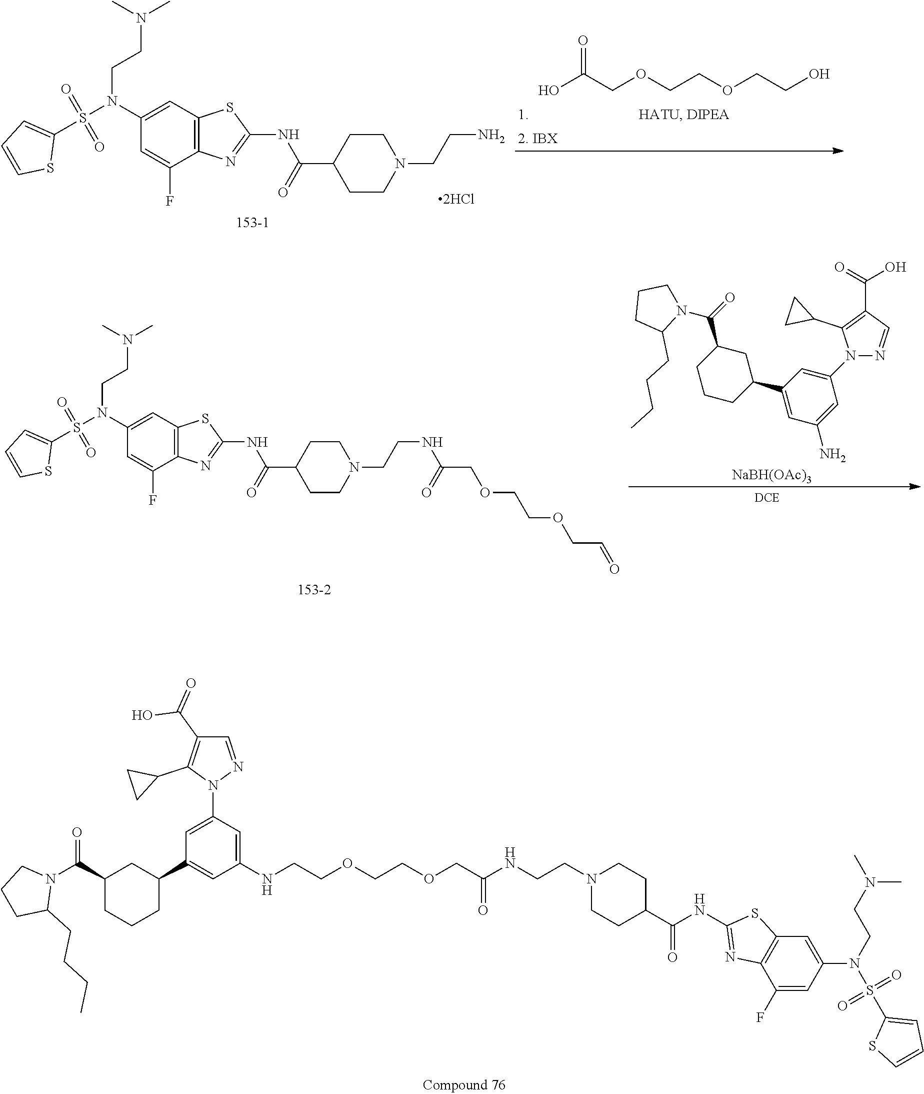 [SMILES: CN(C)CCN(c1cc(F)c2nc(NC(=O)C3CCN(CCN)CC3)sc2c1)S(=O)(=O)c1cccs1 ; O=C(O)COCCOCCO ; CN(C)CCN(c1cc(F)c2nc(NC(=O)C3CCN(CCNC(=O)COCCOCC=O)CC3)sc2c1)S(=O)(=O)c1cccs1 ; CCCCC1CCCN1C(=O)[C@@H]1CCC[C@H](c2cc(N)cc(-n3ncc(C(=O)O)c3C3CC3)c2)C1 ; CCCCC1CCCN1C(=O)[C@@H]1CCC[C@H](c2cc(NCCOCCOCC(=O)NCCN3CCC(C(=O)Nc4nc5c(F)cc(N(CCN(C)C)S(=O)(=O)c6cccs6)cc5s4)CC3)cc(-n3ncc(C(=O)O)c3C3CC3)c2)C1 ; "NaBH(OAc)3"]

### Example 154. Synthesis of 1-((4-((8-((2-(4-((6-(N-(2-(dimethylamino)ethyl)thiophene-2-sulfonamido)-4-fluorobenzo[d]thiazol-2-yl)carbamoyl)piperidin-1-yl)ethyl)amino)-8-oxooctyl)amino)phenyl)sulfonyl)-5-((4-ethoxyphenyl)sulfonamido)-N-hydroxy-2-methyl-1H-benzo[g]indole-3-carboxamide Compound 77

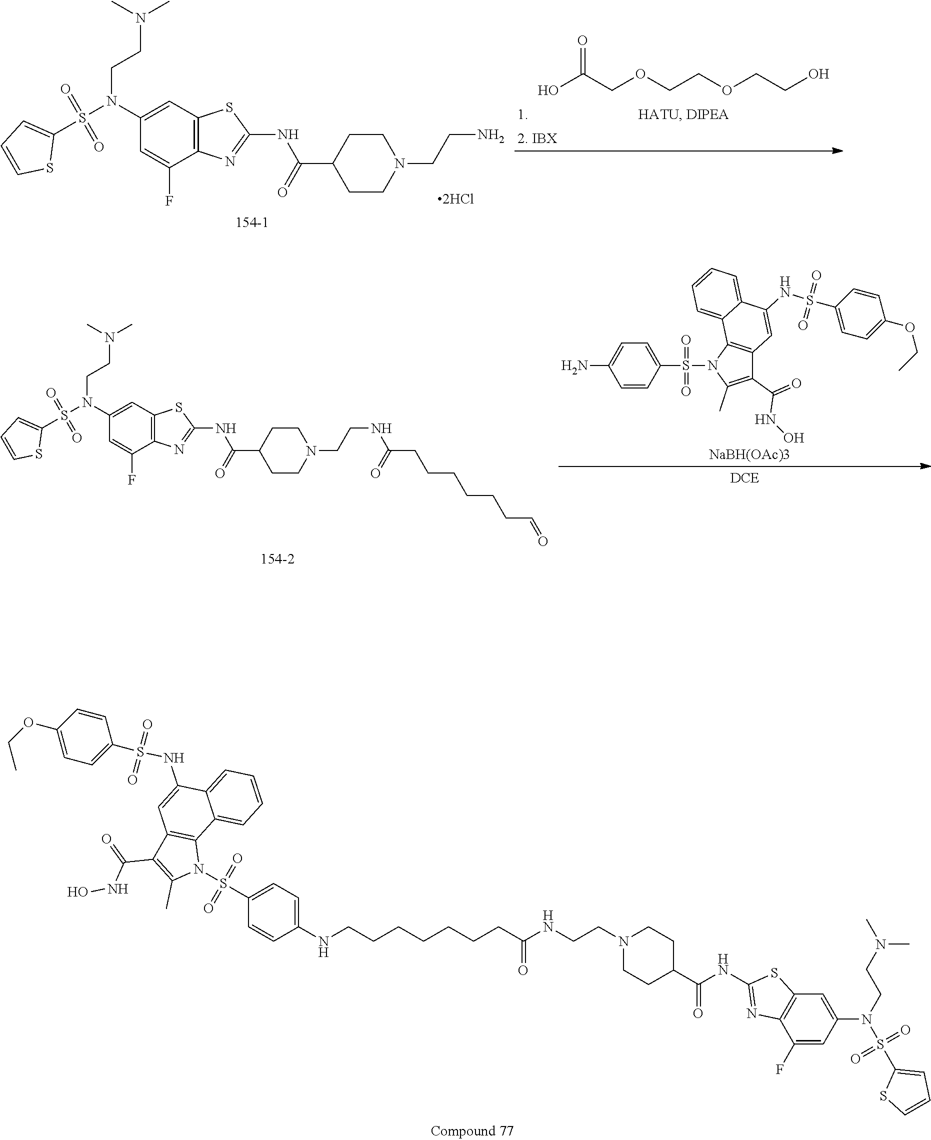 [SMILES: CN(C)CCN(c1cc(F)c2nc(NC(=O)C3CCN(CCN)CC3)sc2c1)S(=O)(=O)c1cccs1 ; O=C(O)COCCOCCO ; CN(C)CCN(c1cc(F)c2nc(NC(=O)C3CCN(CCNC(=O)CCCCCCC=O)CC3)sc2c1)S(=O)(=O)c1cccs1 ; CCOc1ccc(S(=O)(=O)Nc2cc3c(C(=O)NO)c(C)n(S(=O)(=O)c4ccc(N)cc4)c3c3ccccc23)cc1 ; CCOc1ccc(S(=O)(=O)Nc2cc3c(C(=O)NO)c(C)n(S(=O)(=O)c4ccc(NCCCCCCCC(=O)NCCN5CCC(C(=O)Nc6nc7c(F)cc(N(CCN(C)C)S(=O)(=O)c8cccs8)cc7s6)CC5)cc4)c3c3ccccc23)cc1 ; "NaBH(OAc)3"]

### Example 155. Synthesis of N1-(2-(4-((6-(N-(2-(dimethylamino)ethyl)thiophene-2-sulfonamido)-4-fluorobenzo[d]thiazol-2-yl)carbamoyl)piperidin-1-yl)ethyl)-N8-((5-((4-(((1r,4r)-4-morpholinocyclohexyl)oxy)furo[3,2-d]pyrimidin-2-yl)amino)isothiazol-3-yl)methyl)octanediamide Compound 78

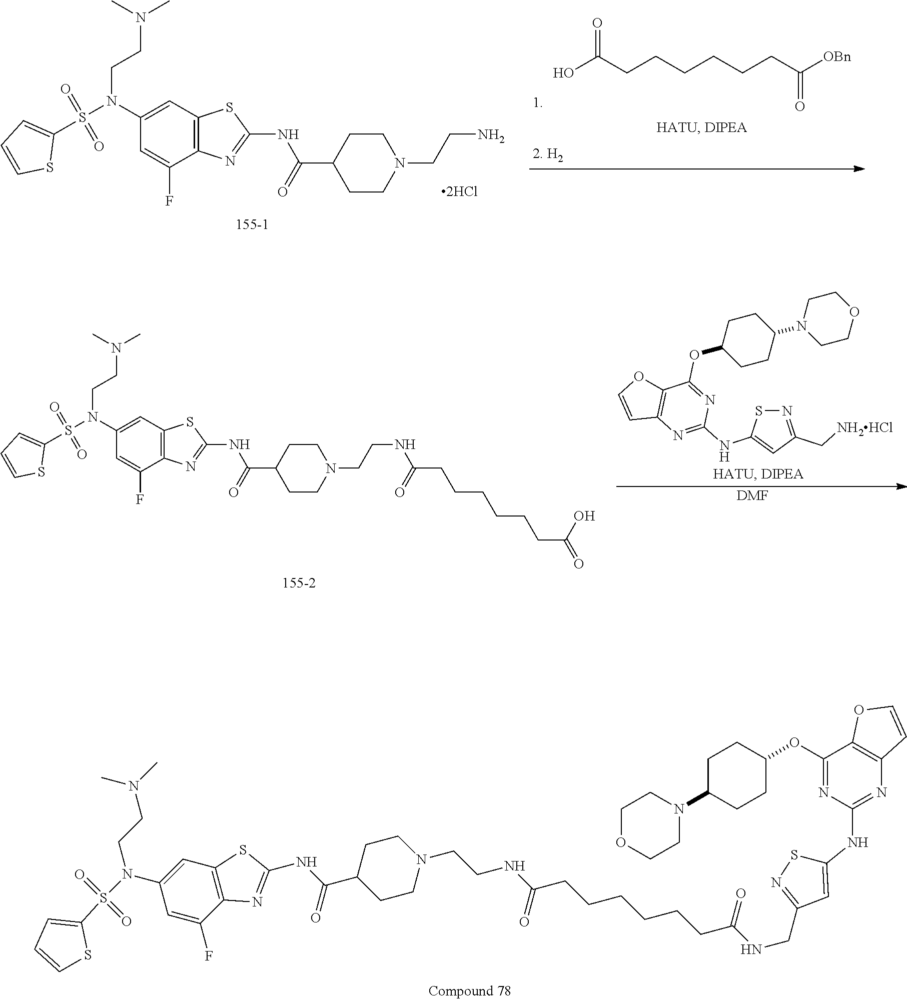

### Example 156. Synthesis of N-(6-(N-(2-hydroxyethyl)thiophene-2-sulfonamido)benzo[d]thiazol-2-yl)-1-(2-(8-(4-(4-((4-(((1r,4r)-4-morpholinocyclohexyl)oxy)furo[3,2-d]pyrimidin-2-yl)amino)-1H-pyrazol-1-yl)piperidin-1-yl)-8-oxooctanamido)ethyl)piperidine-4-carboxamide Compound 79

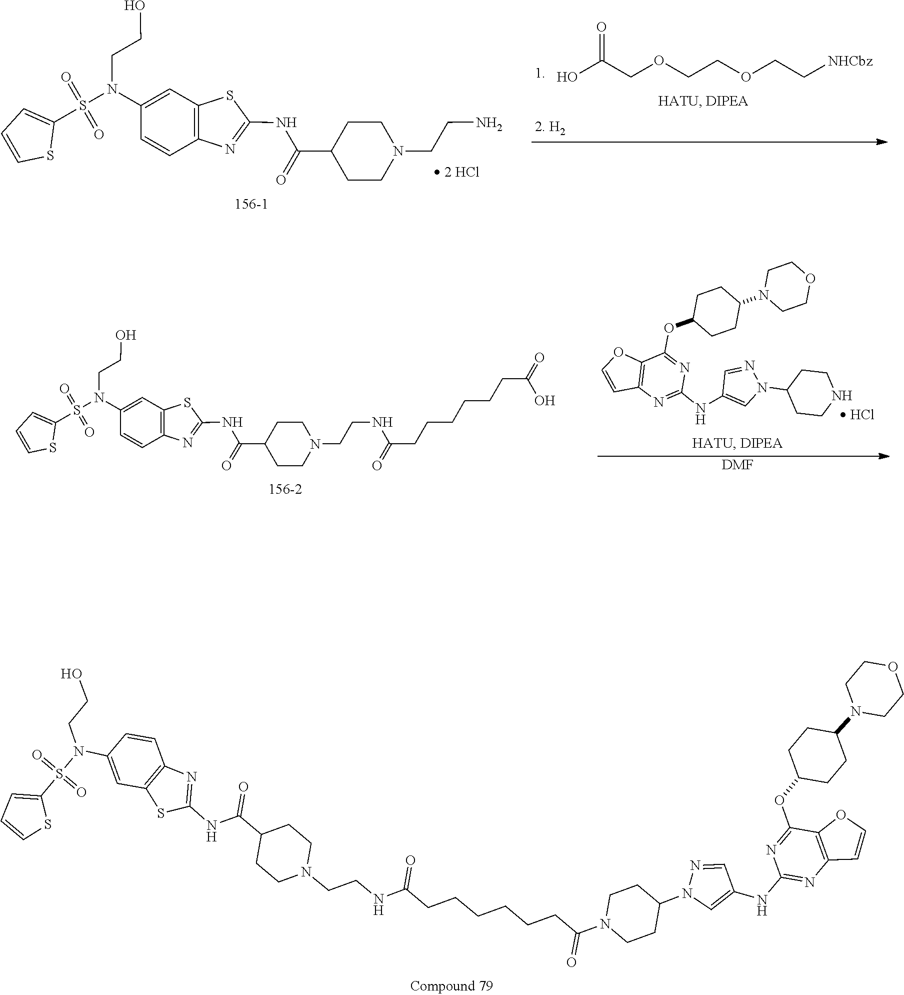

### Example 157 Synthesis of (Z)-1-((E)-4-((Z)-5-carbamoyl-2-((1-ethyl-3-methyl-1H-pyrazole-5-carbonyl)imino)-7-(3-(4-(8-oxo-8-((2-(4-((6-(thiophene-2-sulfonamido)benzo[d]thiazol-2-yl)carbamoyl)piperidin-1-yl)ethyl)amino)octyl)piperazin-1-yl)propoxy)-2,3-dihydro-1H-benzo[d]imidazol-1-yl)but-2-en-1-yl)-2-((1-ethyl-3-methyl-1H-pyrazole-5-carbonyl)imino)-7-methoxy-2,3-dihydro-1H-benzo[d]imidazole-5-carboxamide Compound 80

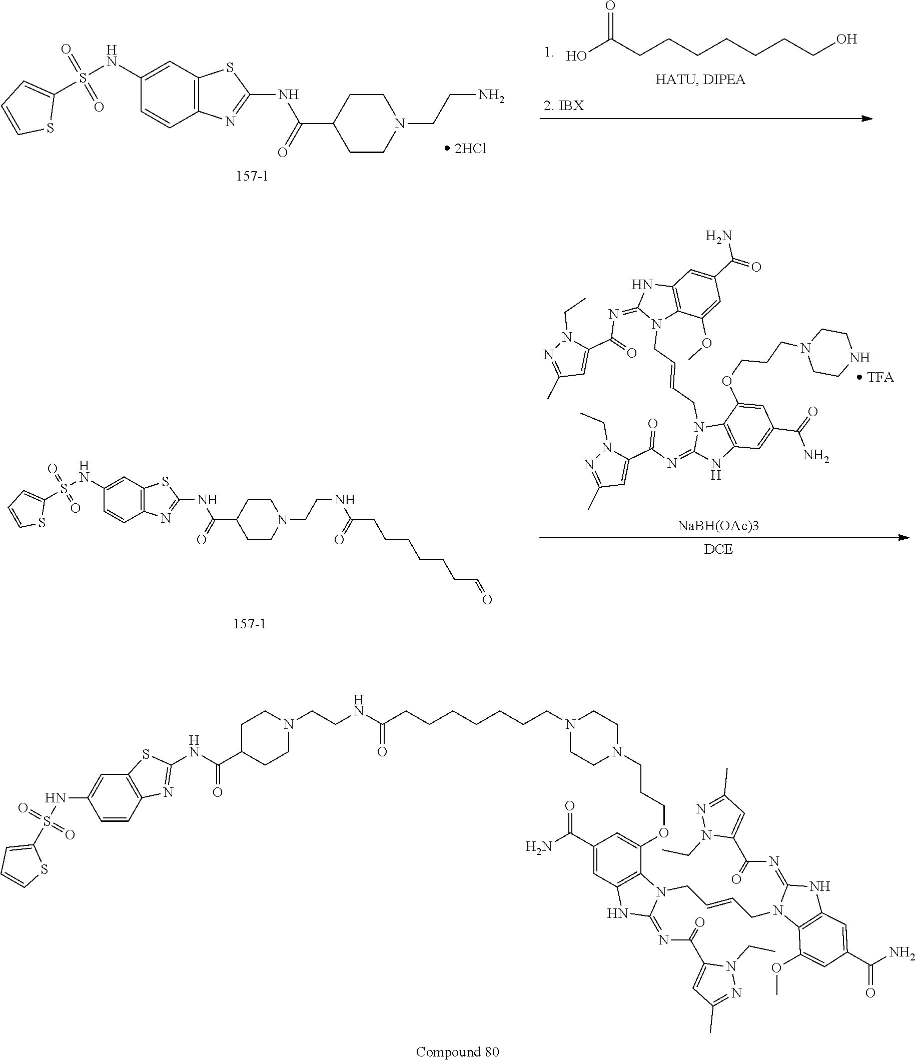 [SMILES: NCCN1CCC(C(=O)Nc2nc3ccc(NS(=O)(=O)c4cccs4)cc3s2)CC1 ; O=C(O)CCCCCCCO ; O=CCCCCCCC(=O)NCCN1CCC(C(=O)Nc2nc3ccc(NS(=O)(=O)c4cccs4)cc3s2)CC1 ; CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCNCC3)c21 ; CCn1nc(C)cc1C(=O)/N=c1/[nH]c2cc(C(N)=O)cc(OC)c2n1C/C=C/Cn1/c(=N\C(=O)c2cc(C)nn2CC)[nH]c2cc(C(N)=O)cc(OCCCN3CCN(CCCCCCCC(=O)NCCN4CCC(C(=O)Nc5nc6ccc(NS(=O)(=O)c7cccs7)cc6s5)CC4)CC3)c21 ; "NaBH(OAc)3"]

### Example 158 Synthesis of 1-(2-(8-((9,10-dioxo-7-pivalamido-9,10-dihydrophenanthren-3-yl)amino)octanamido)ethyl)-N-(6-(thiophene-2-sulfonamido)thiazolo[4,5-b]pyridin-2-yl)piperidine-4-carboxamide Compound 81

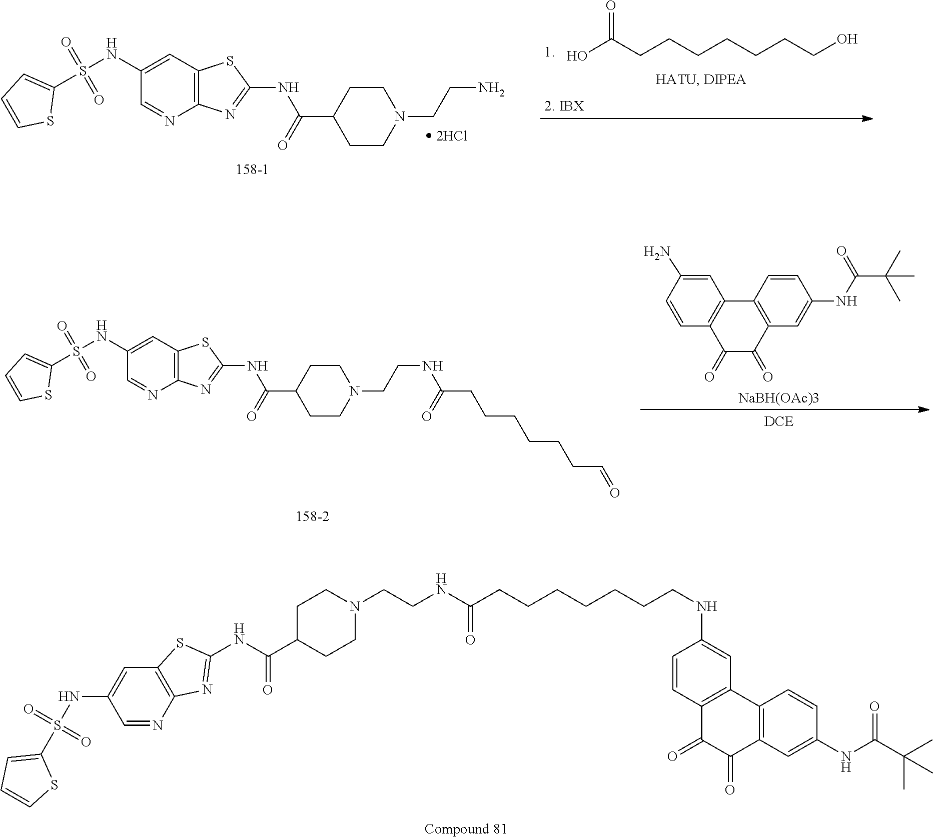

### Example 159. Synthesis of 1-(2-(7-(1-((2S,3R,5R)-5-(5-((E)-2-bromovinyl)-2,4-dioxo-3,4-dihydropyrimidin-1(2H)-yl)-2-(hydroxymethyl)tetrahydrofuran-3-yl)-1H-1,2,3-triazol-4-yl)heptanamido)ethyl)-N-(4-fluoro-6-(N-(2-hydroxyethyl)thiophene-2-sulfonamido)benzo[d]thiazol-2-yl)piperidine-4-carboxamide Compound 82

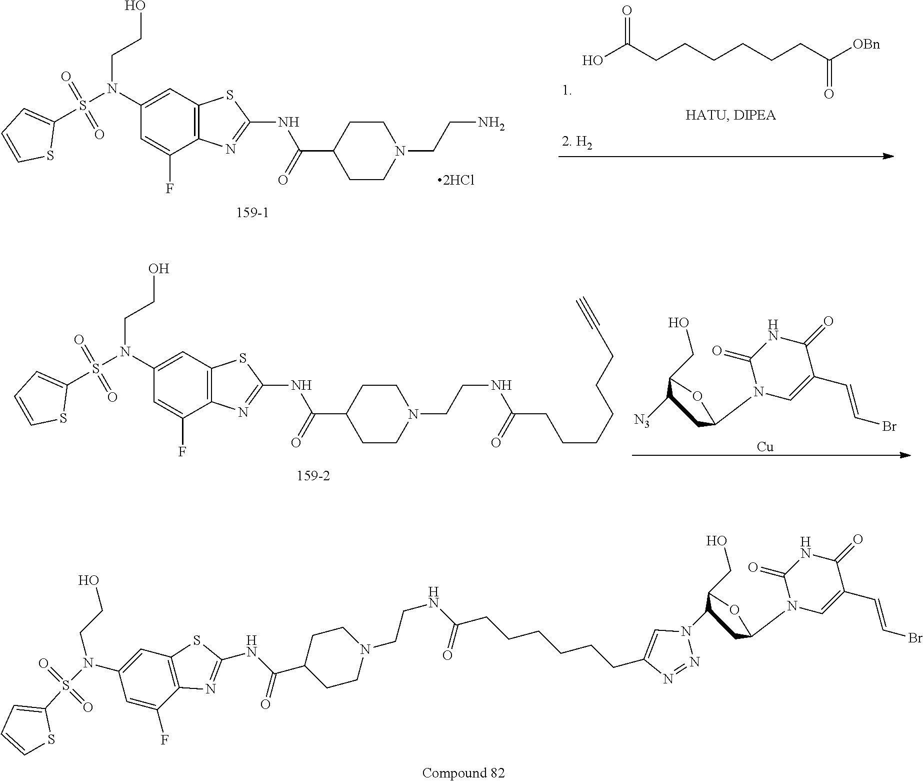

### Example 160. Synthesis of (R)—N-(4-(4-methoxyphenyl)thiazol-2-yl)-1-((4-(((8-oxo-8-((2-(4-((5-(thiophene-2-sulfonamido)thiazolo[5,4-b]pyridin-2-yl)carbamoyl)piperidin-1-yl)ethyl)amino)octyl)amino)methyl)phenyl)sulfonyl)piperidine-2-carboxamide Compound

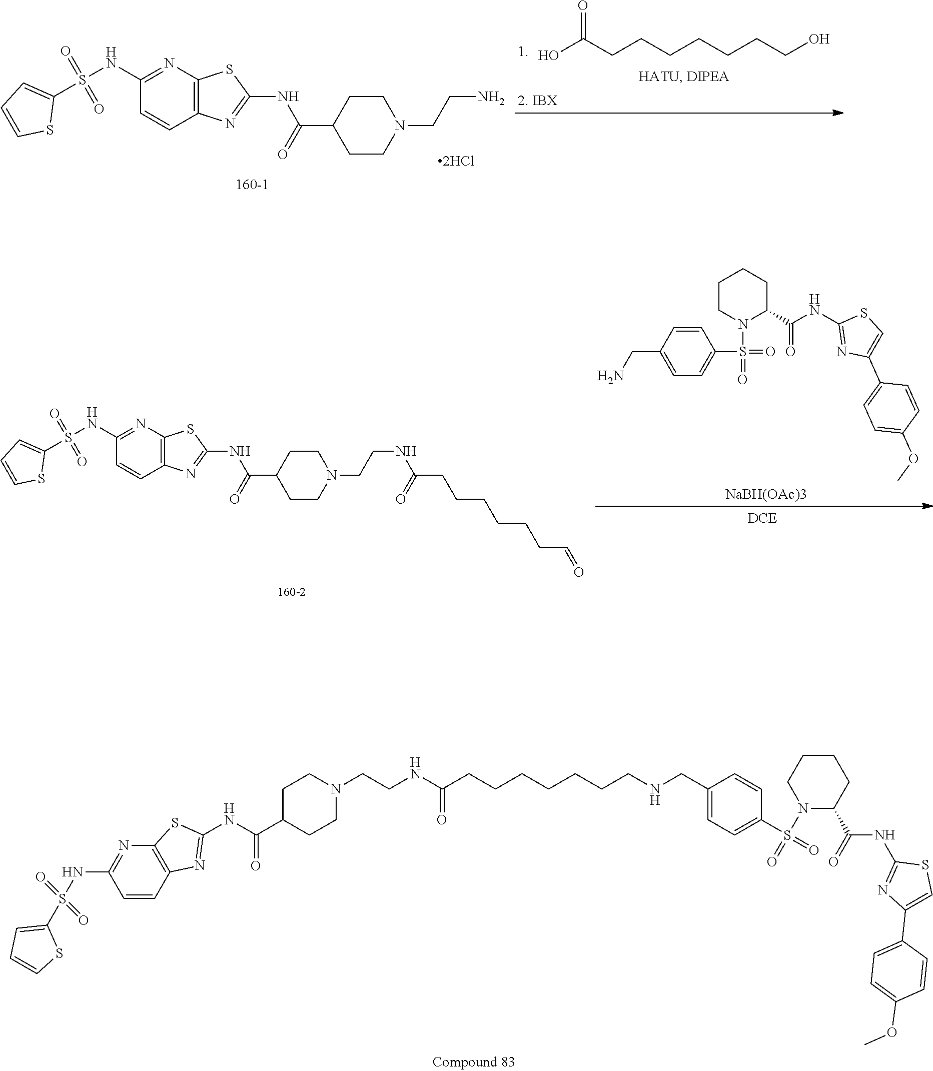

### Example 161. Synthesis of Synthesis of (S)-4-(acrylamidomethyl)-N-(2-(4-((15-(3-((4-hydroxy-1-(3-phenylbutanoyl)piperidin-4-yl)methyl)-4-oxo-3,4-dihydroquinazolin-7-yl)-11-oxo-3,6,9-trioxa-12-azapentadec-14-yn-1-yl)oxy)phenoxy)phenyl)benzamide Compound 84

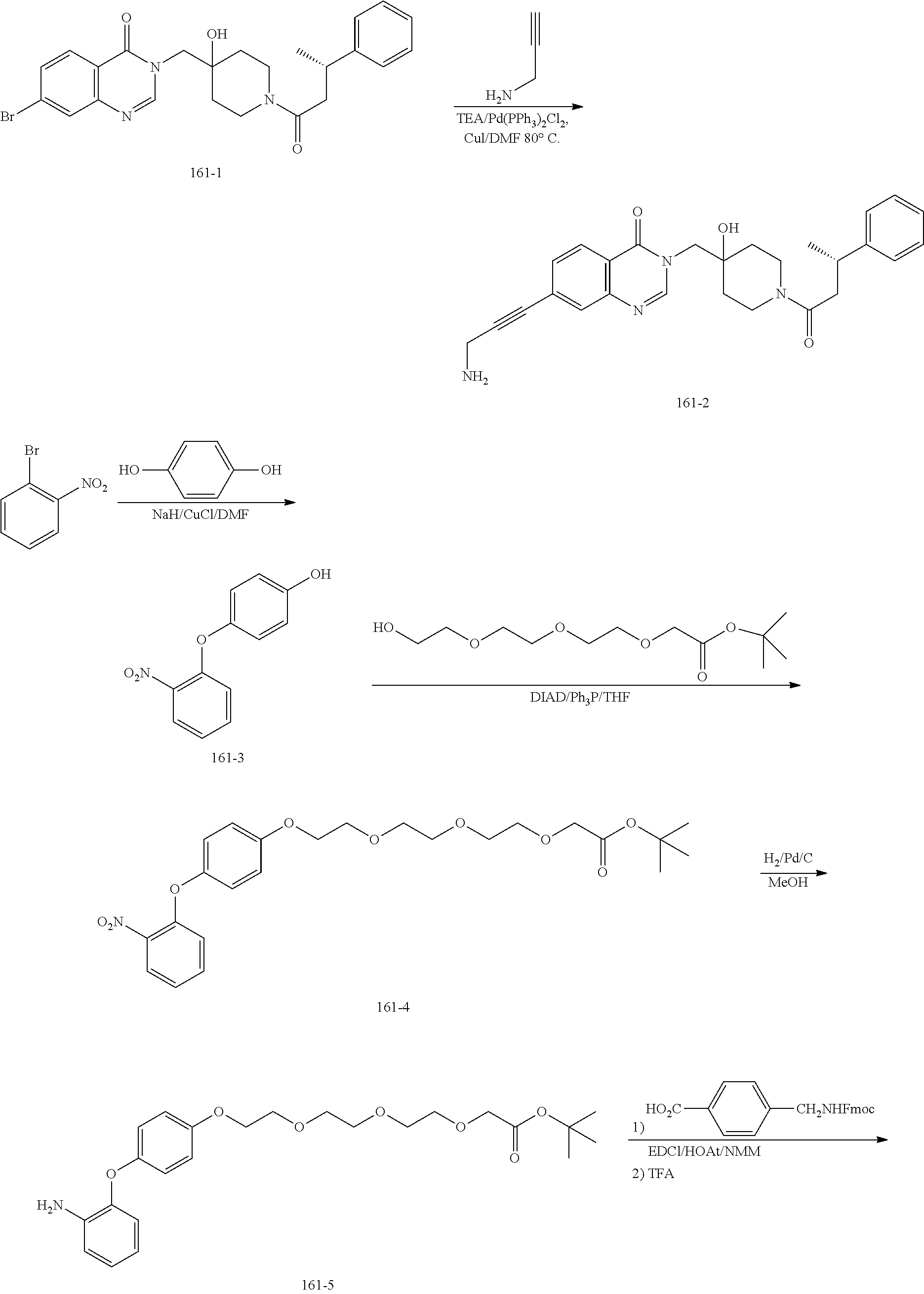

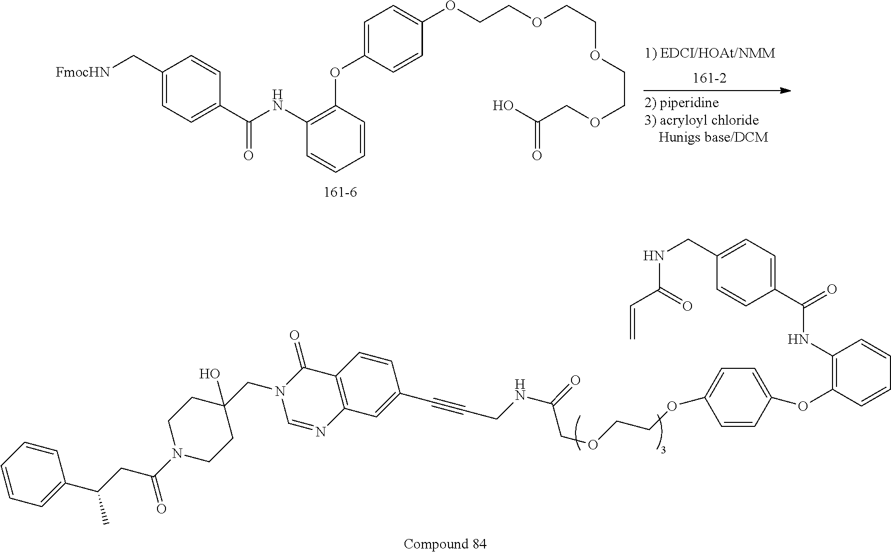

### Example 162. Synthesis of (S)-4-(acrylamidomethyl)-N-(2-(4-((5-((4-(((3-(3-((4-hydroxy-1-(3-phenylbutanoyl)piperidin-4-yl)methyl)-4-oxo-3,4-dihydroquinazolin-7-yl)prop-2-yn-1-yl)amino)methyl)-1H-pyrazol-1-yl)methyl)pyrazin-2-yl)methoxy)phenoxy)phenyl)benzamide Compound 85

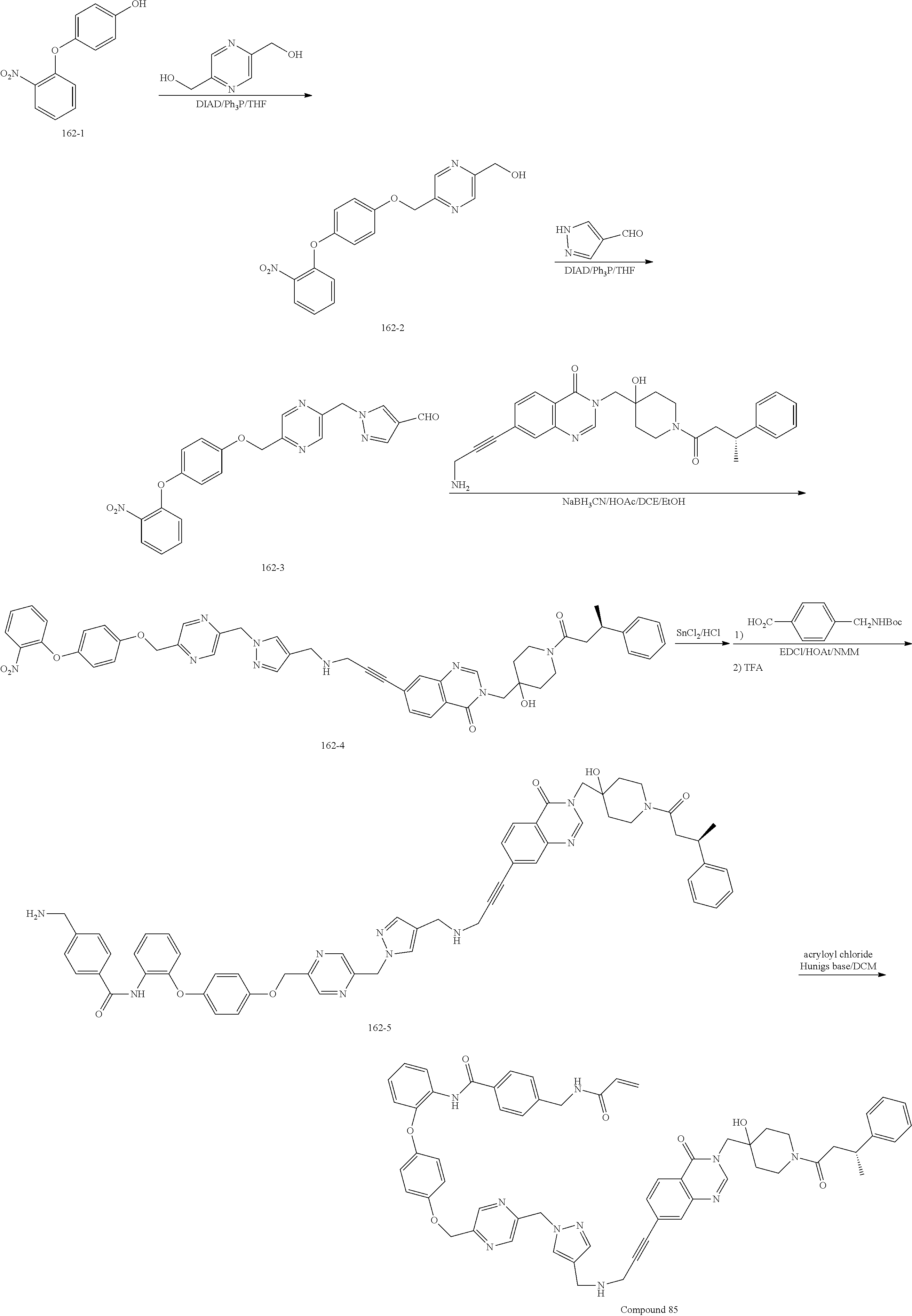

### Example 163. Synthesis of (R)-4-(acrylamidomethyl)-N-(2-(4-((5-((4-(((3-(3-((4-hydroxy-1-(3-phenylbutanoyl)piperidin-4-yl)methyl)-4-oxo-3,4-dihydroquinazolin-7-yl)propyl)amino)methyl)-1H-pyrazol-1-yl)methyl)pyrazin-2-yl)methoxy)phenoxy)phenyl)benzamide Compound 86

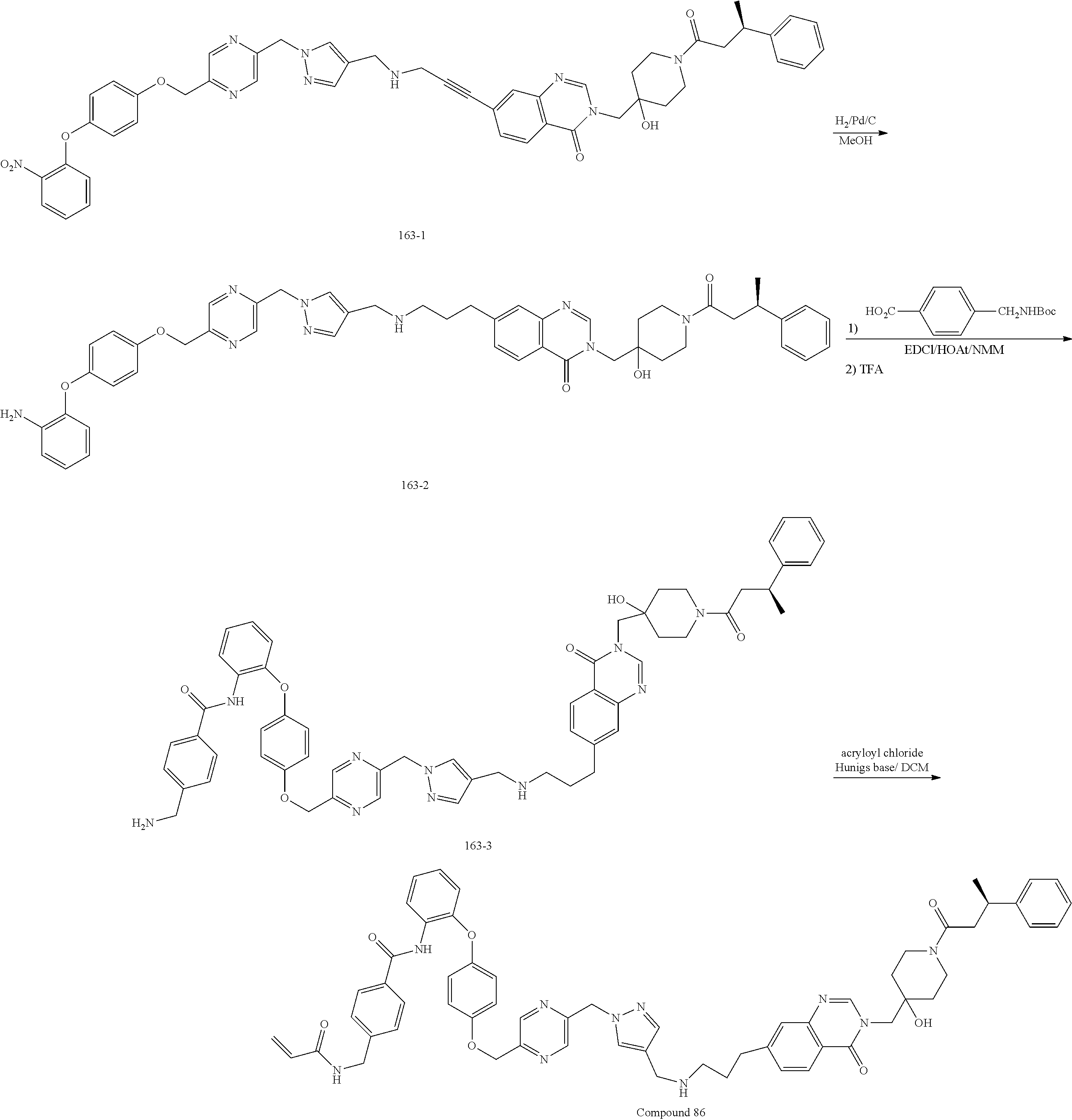

### Example 164. Synthesis of (S)—N-(1-(6′-amino-4′-ethyl-5′-(4-hydroxyphenyl)-[3,3′-bipyridin]-6-yl)-1-oxo-5,8,11-trioxa-2-azatridecan-13-yl)-1′-(2-(4-chlorophenyl)-3-methylbutanoyl)spiro[benzo[d][1,3]dioxole-2,4′-piperidine]-5-carboxamide Compound 87

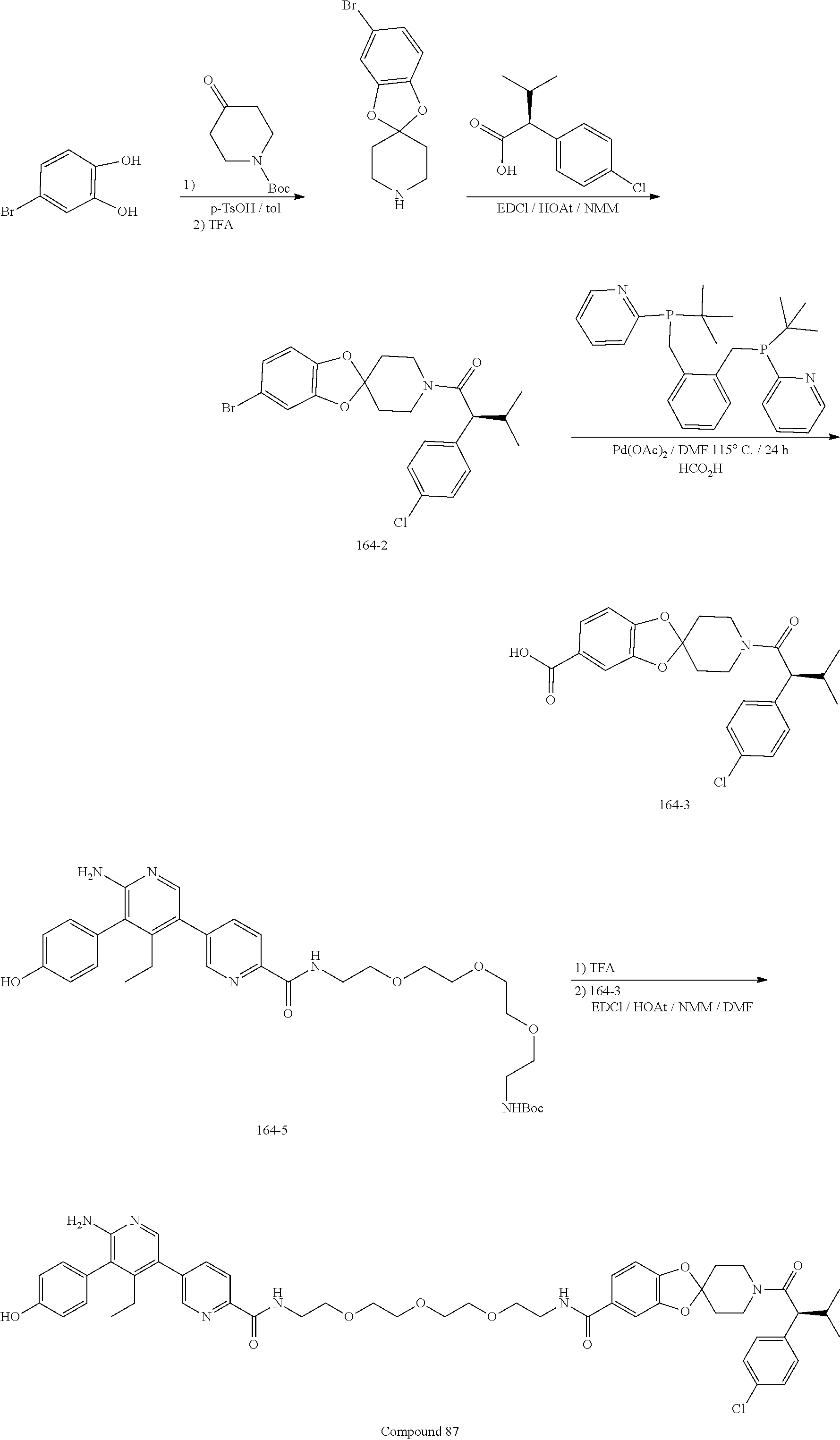

### Example 165. Synthesis of 3-((7-(5-chloro-2-(((2S,6S)-4-((1-(4-((4-(1′-((S)-2-(4-chlorophenyl)-3-methylbutanoyl)spiro[benzo[d][1,3]dioxole-2,4′-piperidin]-5-yl)but-3-yn-1-yl)oxy)butyl)-1H-pyrazol-4-yl)methyl)-2,6-dimethylpiperazin-1-yl)methyl)-3-methylphenyl)thieno[3,2-b]pyridin-2-yl)methyl)-3-azabicyclo[3.1.0]hexane-2,4-dione Compound 88

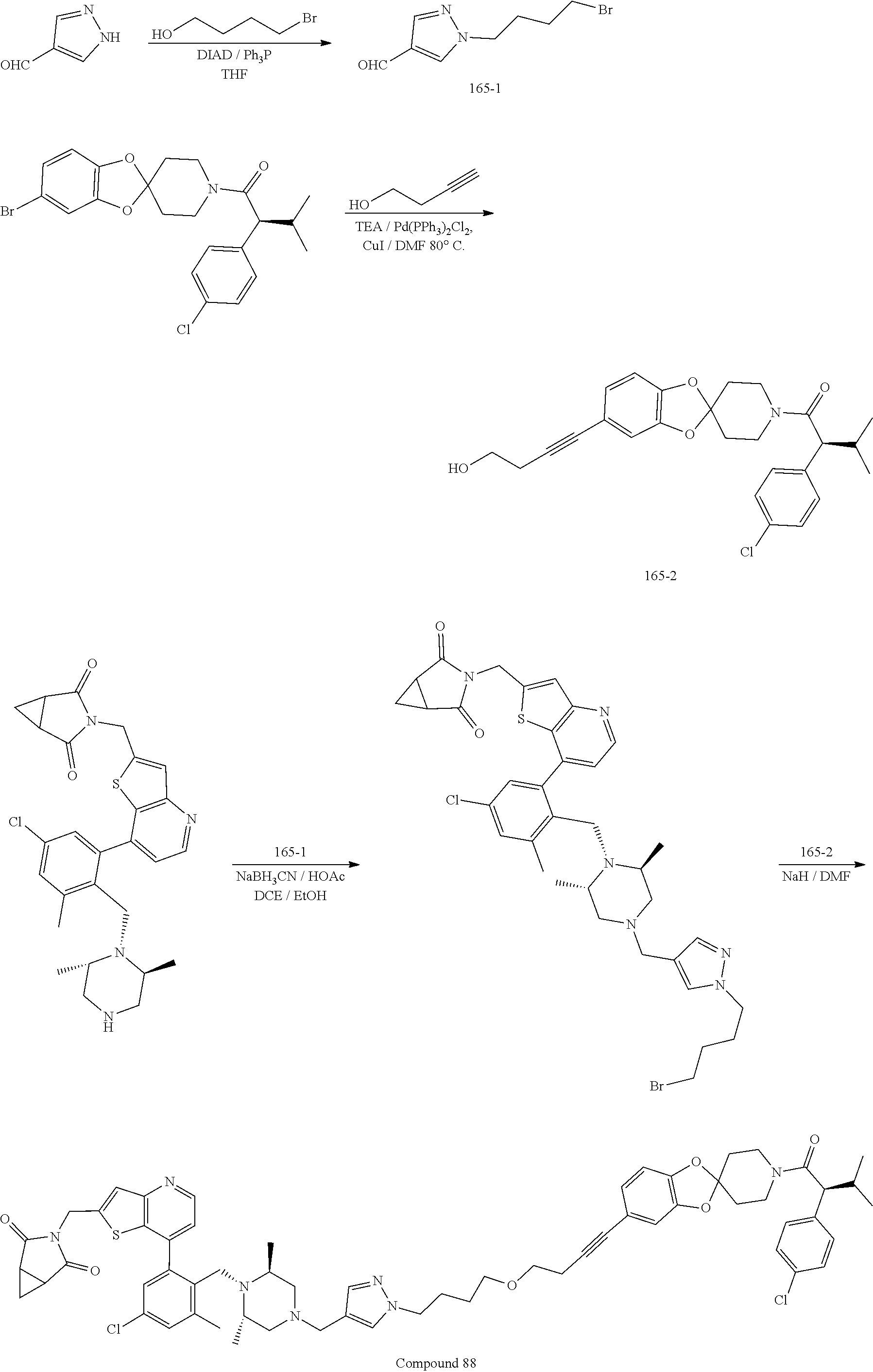

### Example 166. Synthesis of 3-((7-(5-chloro-2-(((2S,6S)-4-((1-(4-(4-(1′-((S)-2-(4-chlorophenyl)-3-methylbutanoyl)spiro[benzo[d][1,3]dioxole-2,4′-piperidin]-5-yl)butoxy)butyl)-1H-pyrazol-4-yl)methyl)-2,6-dimethylpiperazin-1-yl)methyl)-3-methylphenyl)thieno[3,2-b]pyridin-2-yl)methyl)-3-azabicyclo[3.1.0]hexane-2,4-dione Compound 89

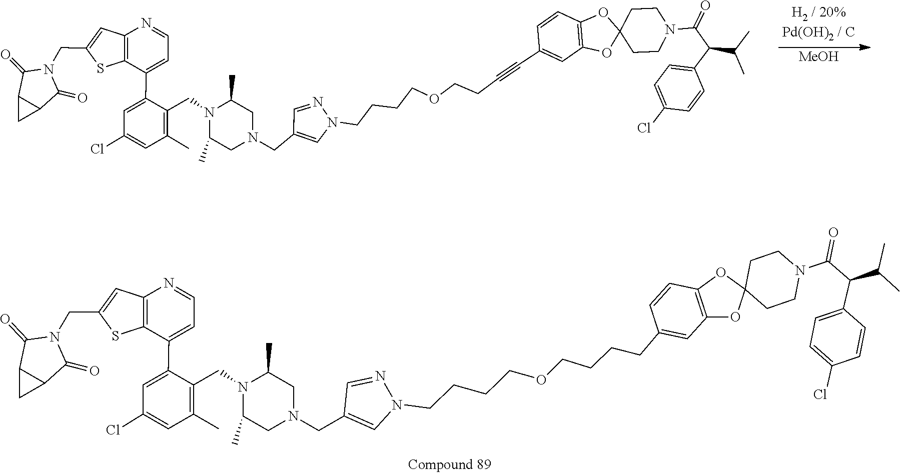

### Example 167. Synthesis of 3-((7-(5-chloro-2-(((2S,6S)-4-((1-(3-(((9-ethyl-7-(4-methylthiophen-2-yl)-9H-carbazol-3-yl)methyl)amino)propyl)-1H-pyrazol-4-yl)methyl)-2,6-dimethylpiperazin-1-yl)methyl)-3-methylphenyl)thieno[3,2-b]pyridin-2-yl)methyl)-3-azabicyclo[3.1.0]hexane-2,4-dione Compound 90

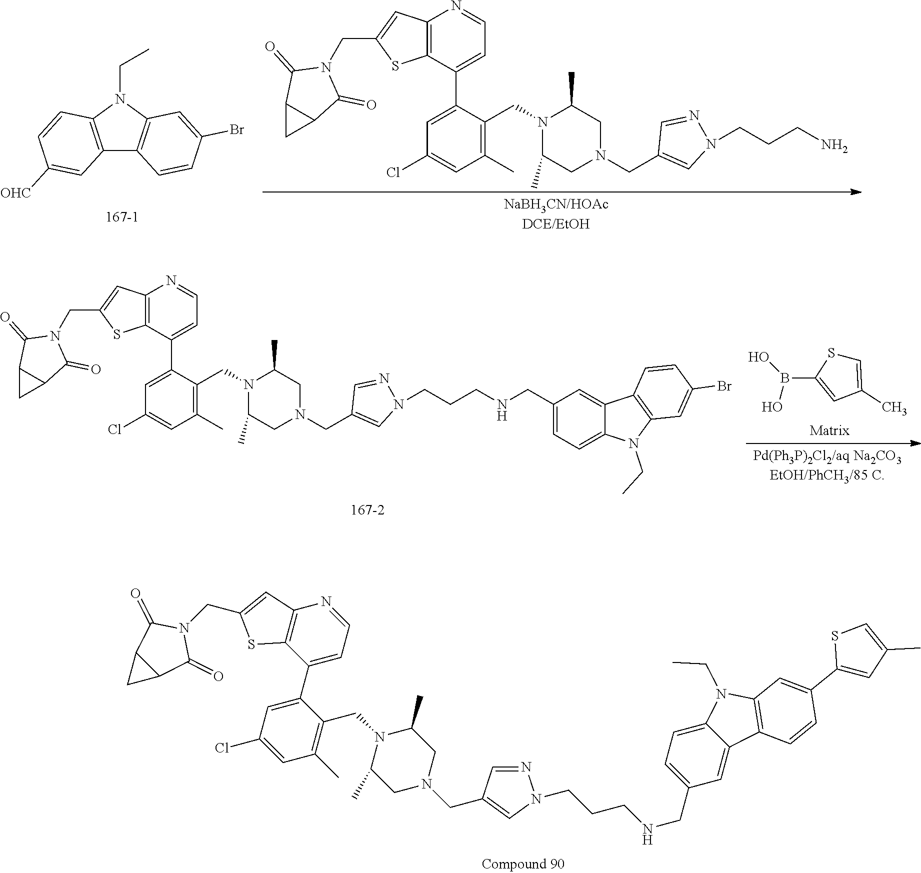

### Example 168. Synthesis of (R)-1-(2-(((9-ethyl-7-(4-methylthiophen-2-yl)-9H-carbazol-3-yl)methyl)amino)ethyl)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)-1H-pyrazole-4-carboxamide Compound 91

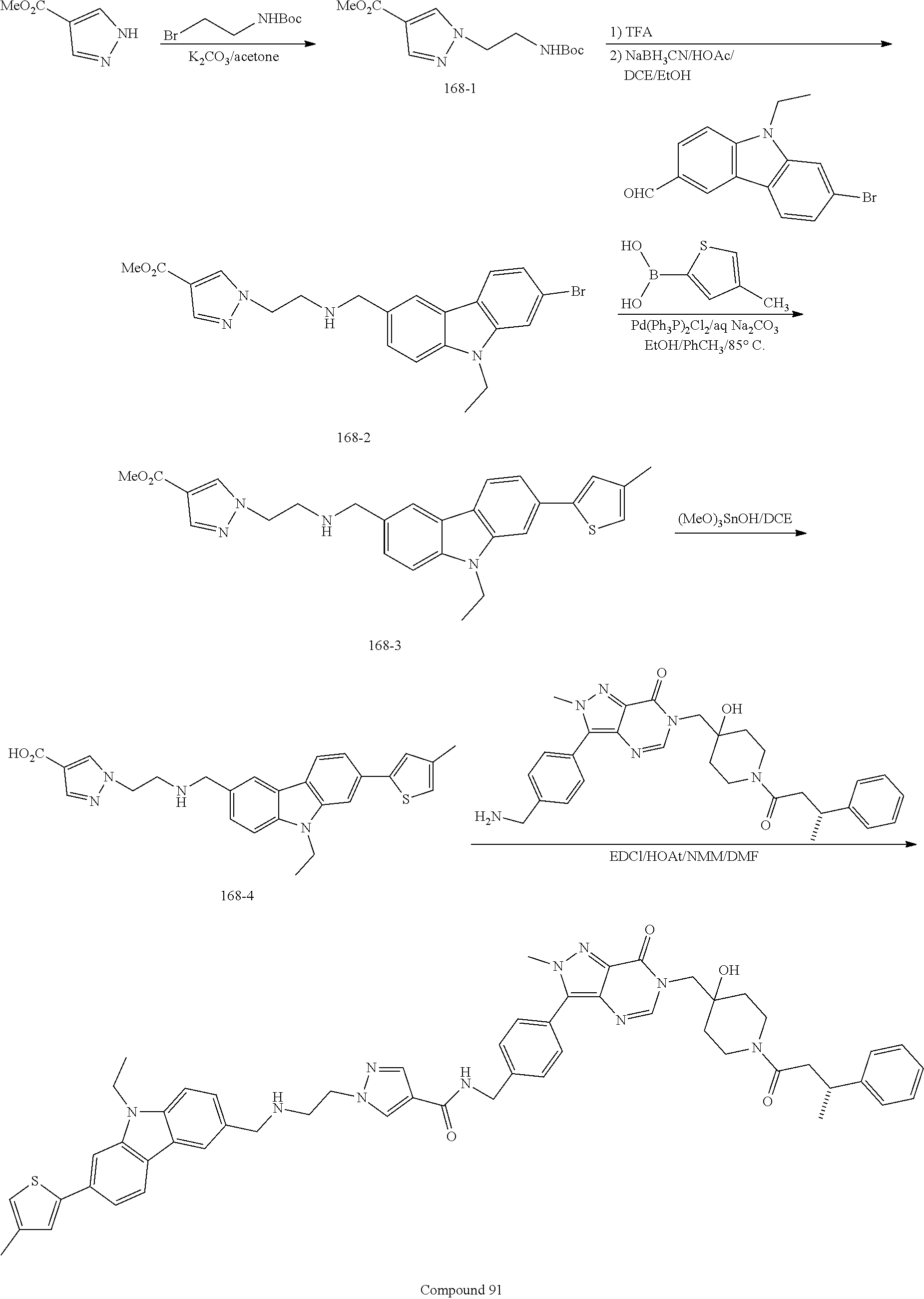

### Example 169. Synthesis of (R)-6′-amino-4′-ethyl-5′-(4-hydroxyphenyl)-N-(15-(4-((3-((5-methyl-4-oxo-2,3,4,5-tetrahydrobenzo[b][1,4]oxazepin-3-yl)carbamoyl)-1H-1,2,4-triazol-5-yl)methyl)phenyl)-13-oxo-3,6,9-trioxa-12-azapentadec-14-yn-1-yl)-[3,3′-bipyridine]-6-carboxamide Compound 92

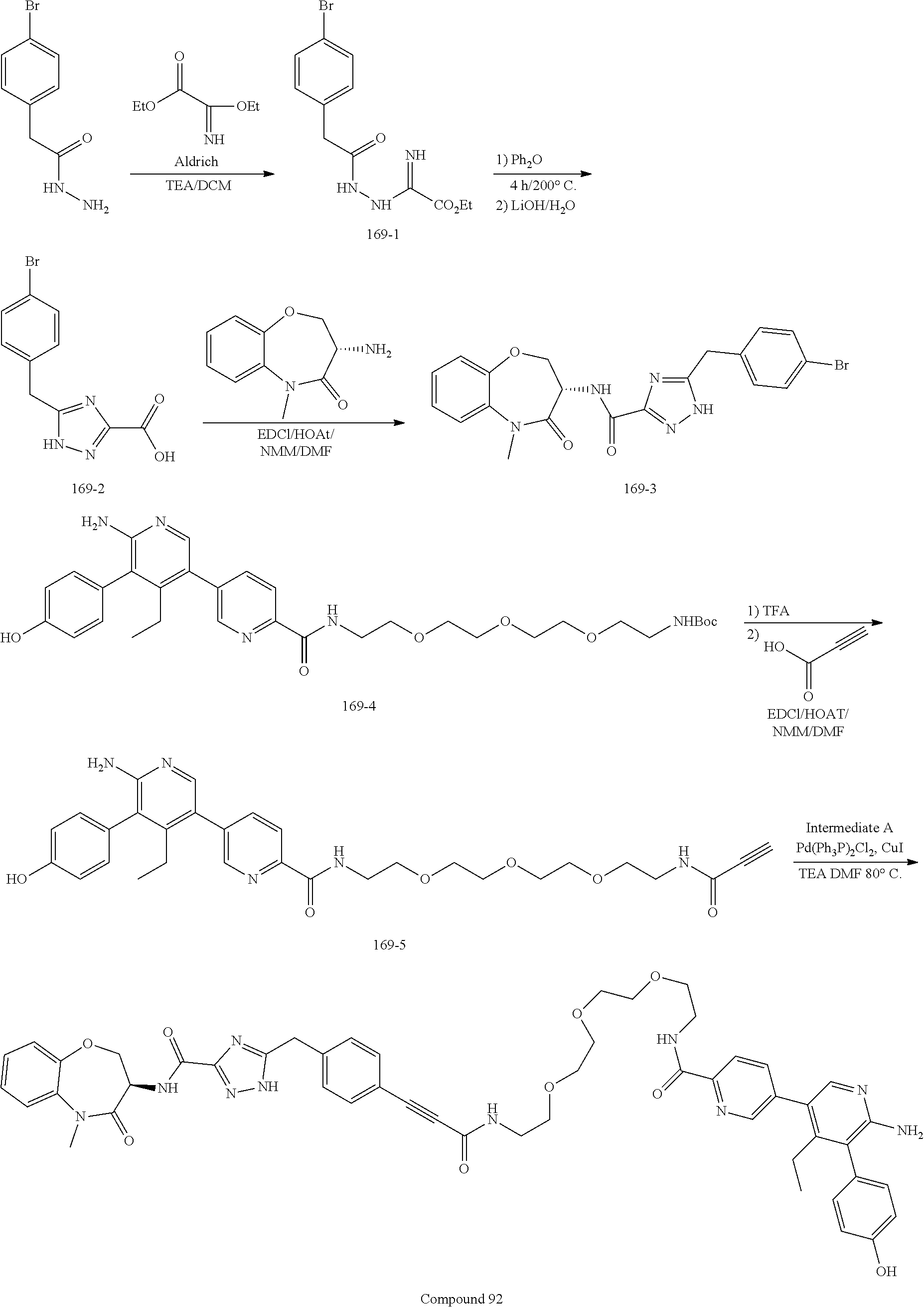

### Example 170: Synthesis of N-(3-(4-(((3R,5R)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-6-((4-((4-((3-(((R)-5-methyl-4-oxo-2,3,4,5-tetrahydrobenzo[b][1,4]oxazepin-3-yl)carbamoyl)-1H-1,2,4-triazol-5-yl)methyl)phenyl)ethynyl)-1H-pyrazol-1-yl)methyl)pyridazine-3-carboxamide Compound 93

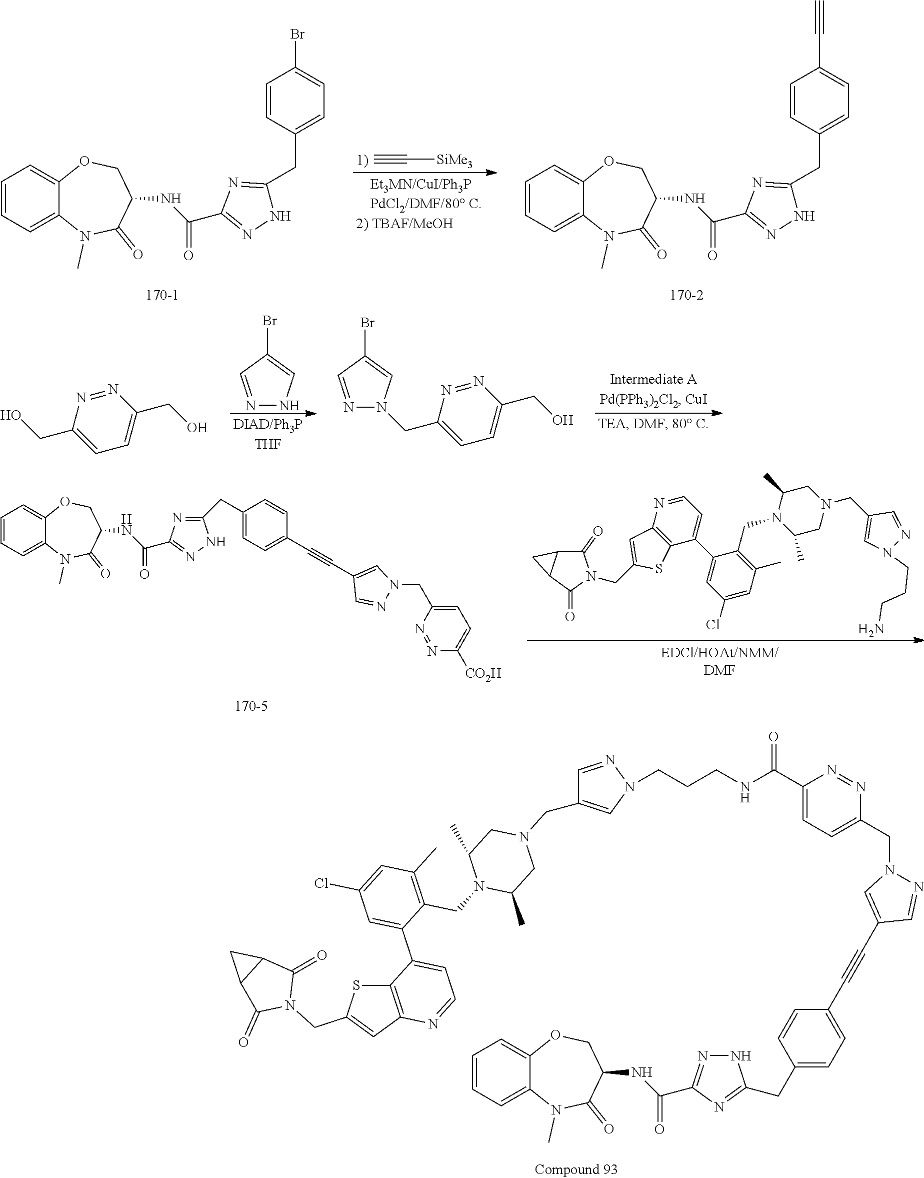

### Example 171. Synthesis of N-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-6-((4-(4-((3-(((R)-5-methyl-4-oxo-2,3,4,5-tetrahydrobenzo[b][1,4]oxazepin-3-yl)carbamoyl)-1H-1,2,4-triazol-5-yl)methyl)phenethyl)-1H-pyrazol-1-yl)methyl)pyridazine-3-carboxamide Compound 94

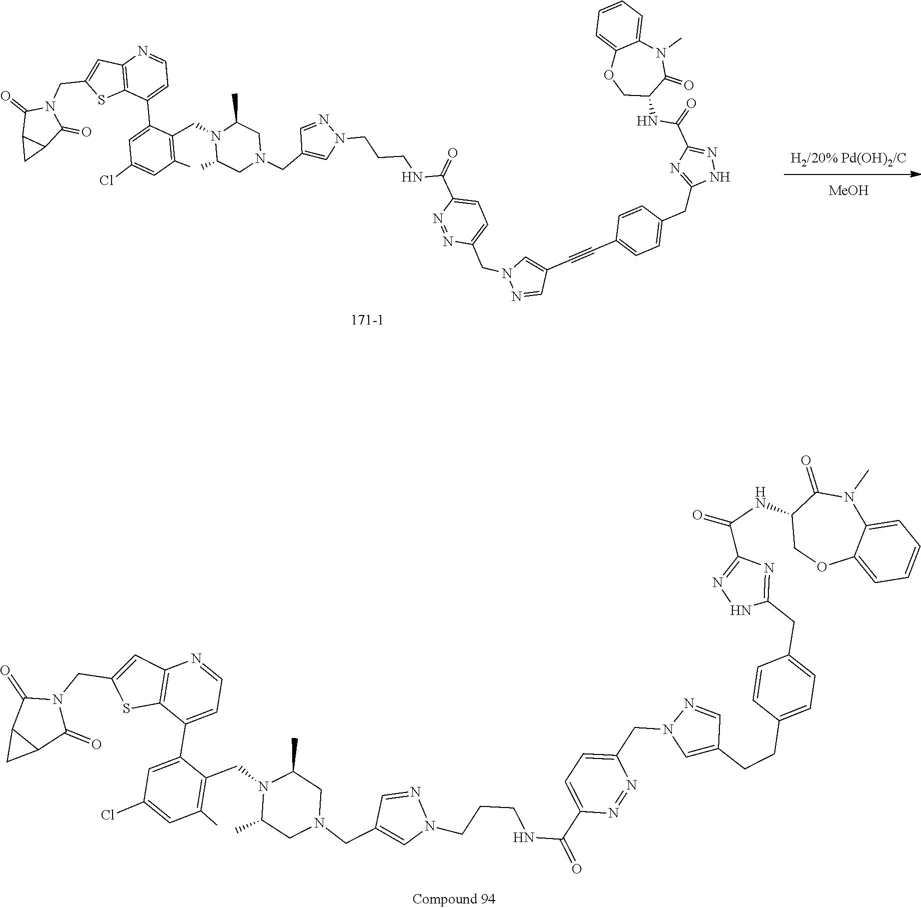

### Example 172. Synthesis of N-(3-(4-(((3R,5R)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-6-((4-((Z)-4-((3-(((S)-5-methyl-4-oxo-2,3,4,5-tetrahydrobenzo[b][1,4]oxazepin-3-yl)carbamoyl)-1H-1,2,4-triazol-5-yl)methyl)styryl)-1H-pyrazol-1-yl)methyl)pyridazine-3-carboxamide Compound 94

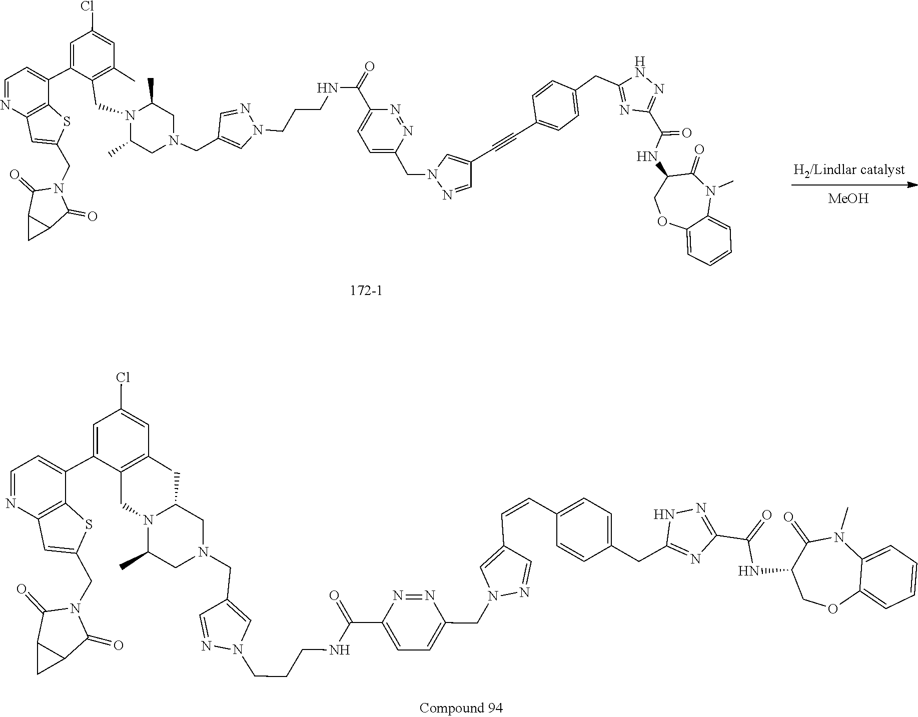

### Example 173. Synthesis of N-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-6-((4-((1S,2R)-2-(4-((3-(((S)-5-methyl-4-oxo-2,3,4,5-tetrahydrobenzo[b][1,4]oxazepin-3-yl)carbamoyl)-1H-1,2,4-triazol-5-yl)methyl)phenyl)cyclopropyl)-1H-pyrazol-1-yl)methyl)pyridazine-3-carboxamide Compound 95

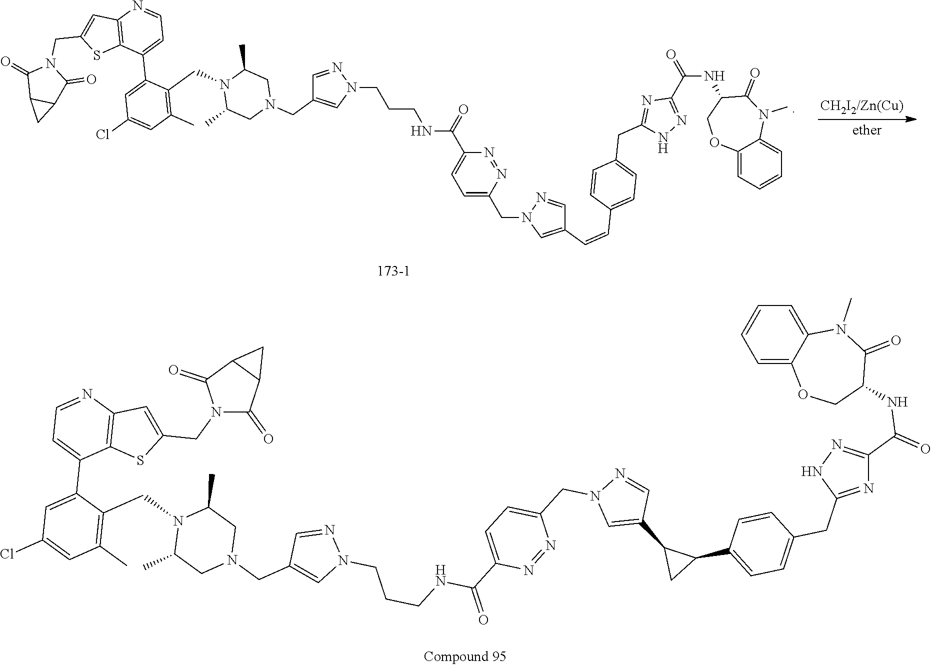

### Example 174. Synthesis of 5-(4-((4-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)butyl)sulfinyl)benzyl)-N—((S)-5-methyl-4-oxo-2,3,4,5-tetrahydrobenzo[b][1,4]oxazepin-3-yl)-1H-1,2,4-triazole-3-carboxamide Compound 96

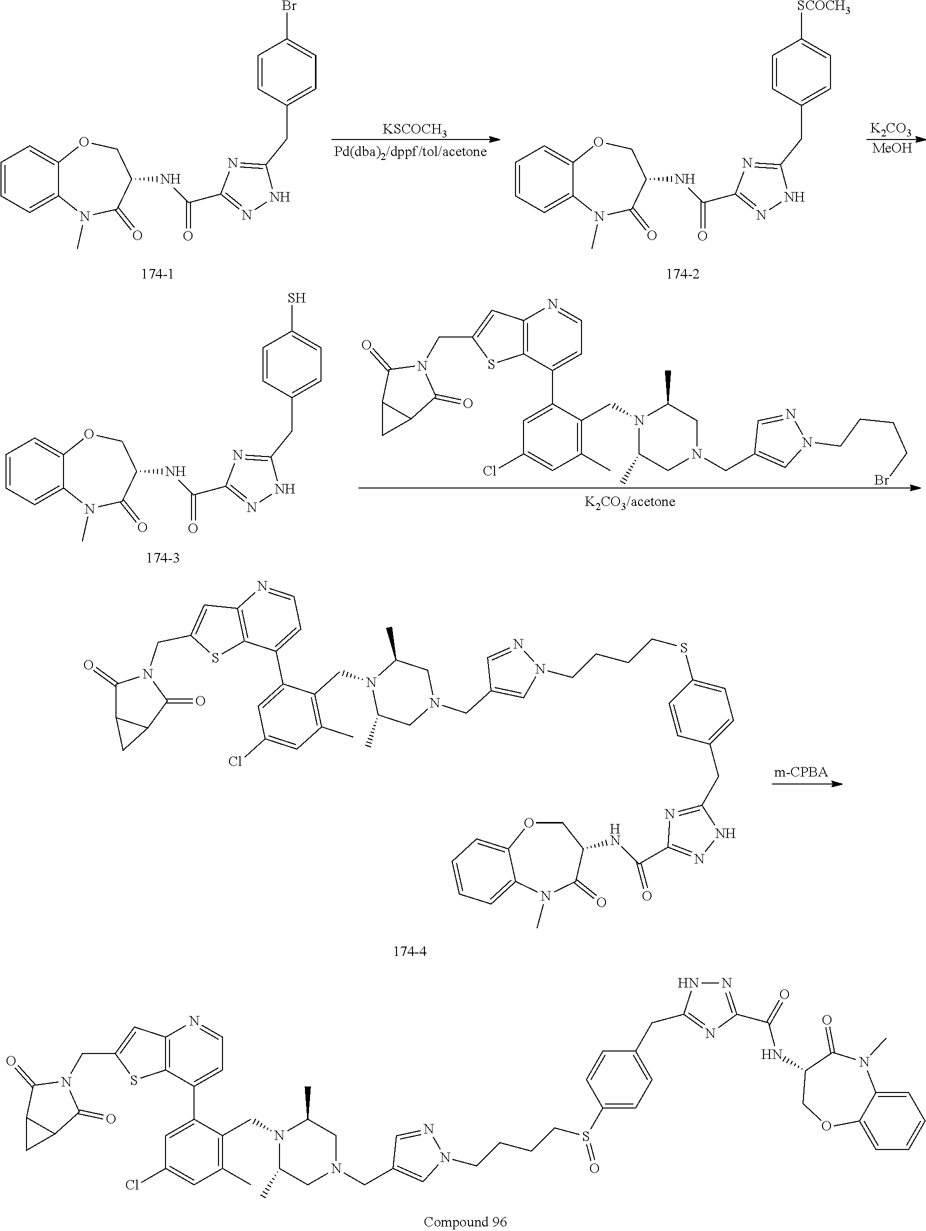

### Example 175: Synthesis of 5-(4-((4-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)butyl)sulfonyl)benzyl)-N—((S)-5-methyl-4-oxo-2,3,4,5-tetrahydrobenzo[b][1,4]oxazepin-3-yl)-1H-1,2,4-triazole-3-carboxamide Compound 97

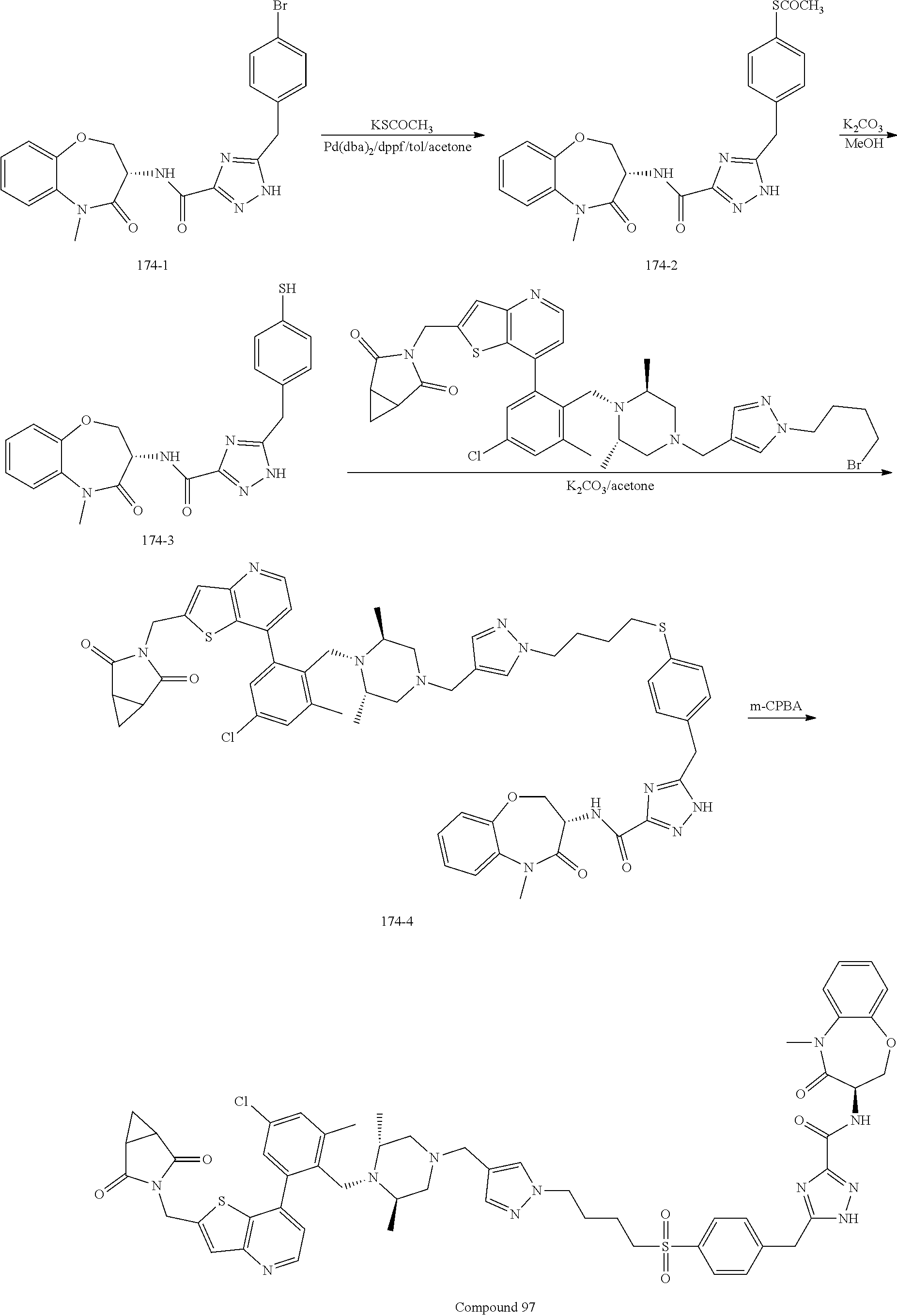

### Example 176: Synthesis of 5-(4-(6-(4-(((3-(4-(((3R,5R)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)amino)methyl)-1H-pyrazol-1-yl)hexa-1,3-diyn-1-yl)benzyl)-N—((S)-5-methyl-4-oxo-2,3,4,5-tetrahydrobenzo[b][1,4]oxazepin-3-yl)-1H-1,2,4-triazole-3-carboxamide Compound 98

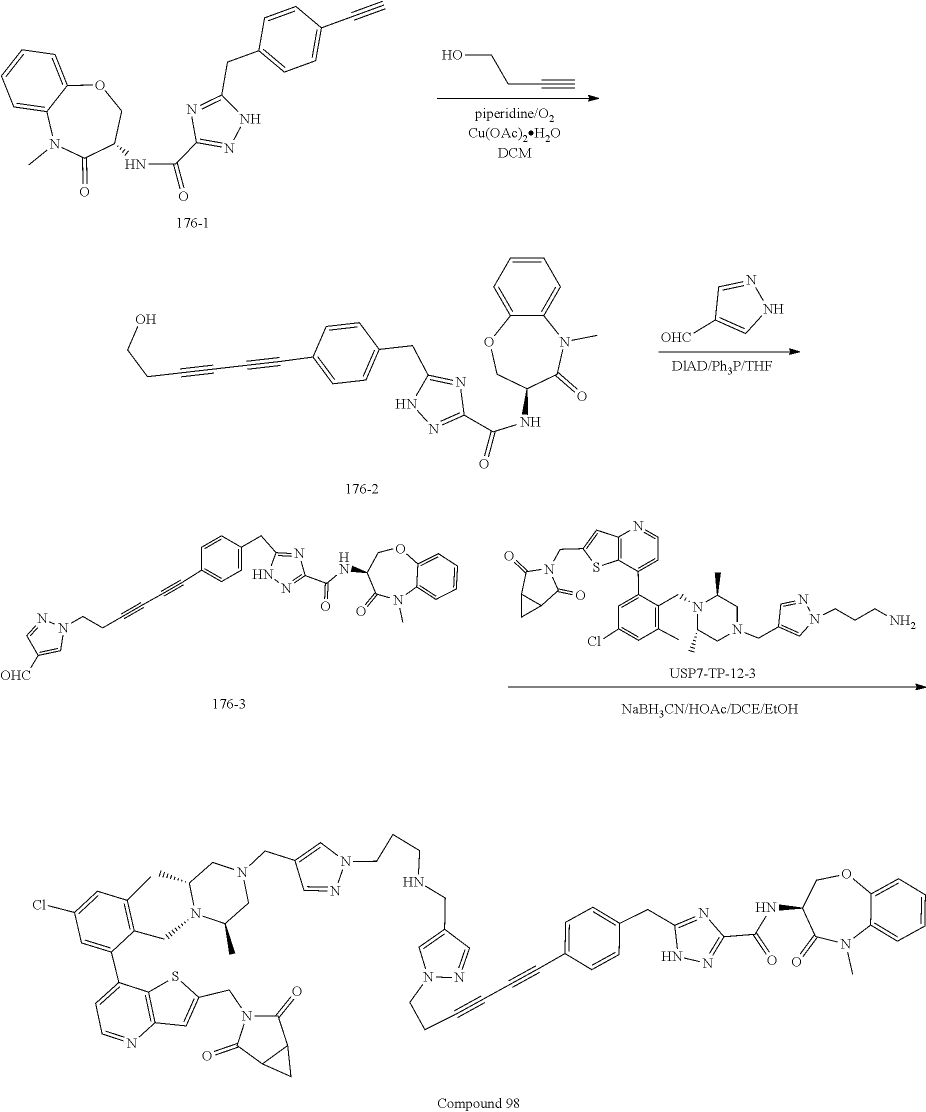

### Example 177: Synthesis of 5-(4-(6-(4-(((4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)amino)methyl)-1H-pyrazol-1-yl)hexa-1,3-diyn-1-yl)benzyl)-N—((S)-5-methyl-4-oxo-2,3,4,5-tetrahydrobenzo[b][1,4]oxazepin-3-yl)-1H-1,2,4-triazole-3-carboxamide Compound 99

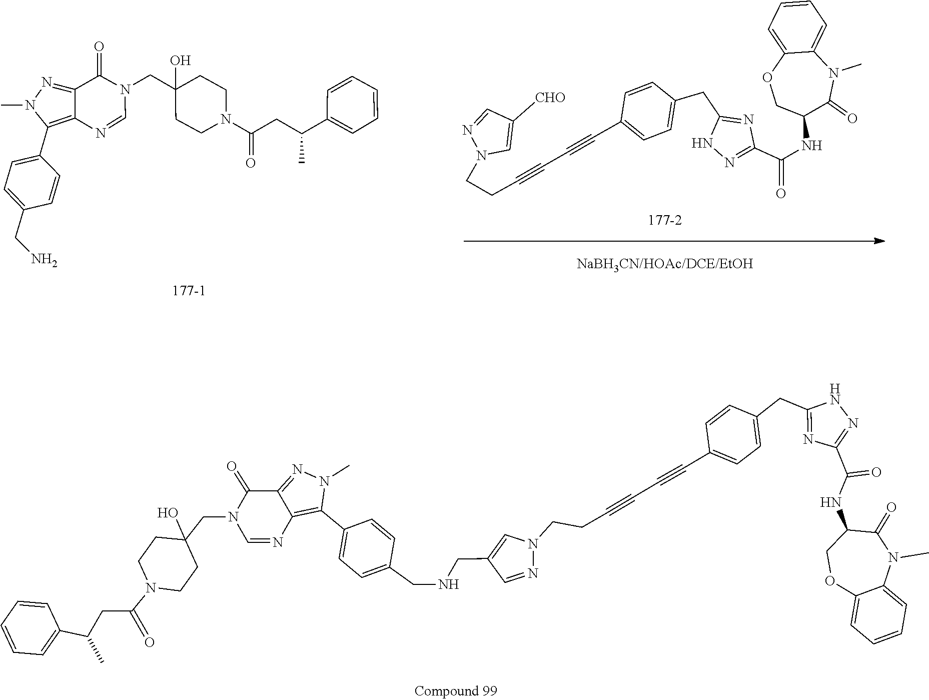

### Example 178: Synthesis of 5-(4-(6-(4-(((4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)amino)methyl)-1H-pyrazol-1-yl)hexyl)benzyl)-N—((S)-5-methyl-4-oxo-2,3,4,5-tetrahydrobenzo[b][1,4]oxazepin-3-yl)-1H-1,2,4-triazole-3-carboxamide Compound 100

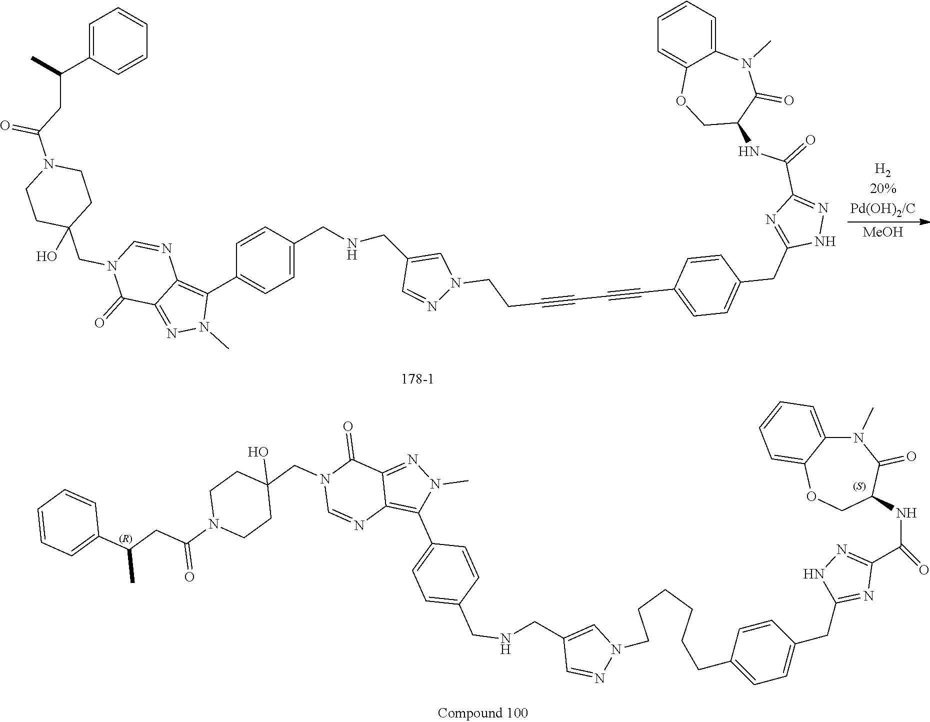

### Example 179: Synthesis of N3-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-N6-(3-(2-mercapto-6-methyl-3-(4-methylpyridin-2-yl)-4-oxo-3,4-dihydrothieno[2,3-d]pyrimidin-5-yl)prop-2-yn-1-yl)pyridazine-3,6-dicarboxamide Compound 101

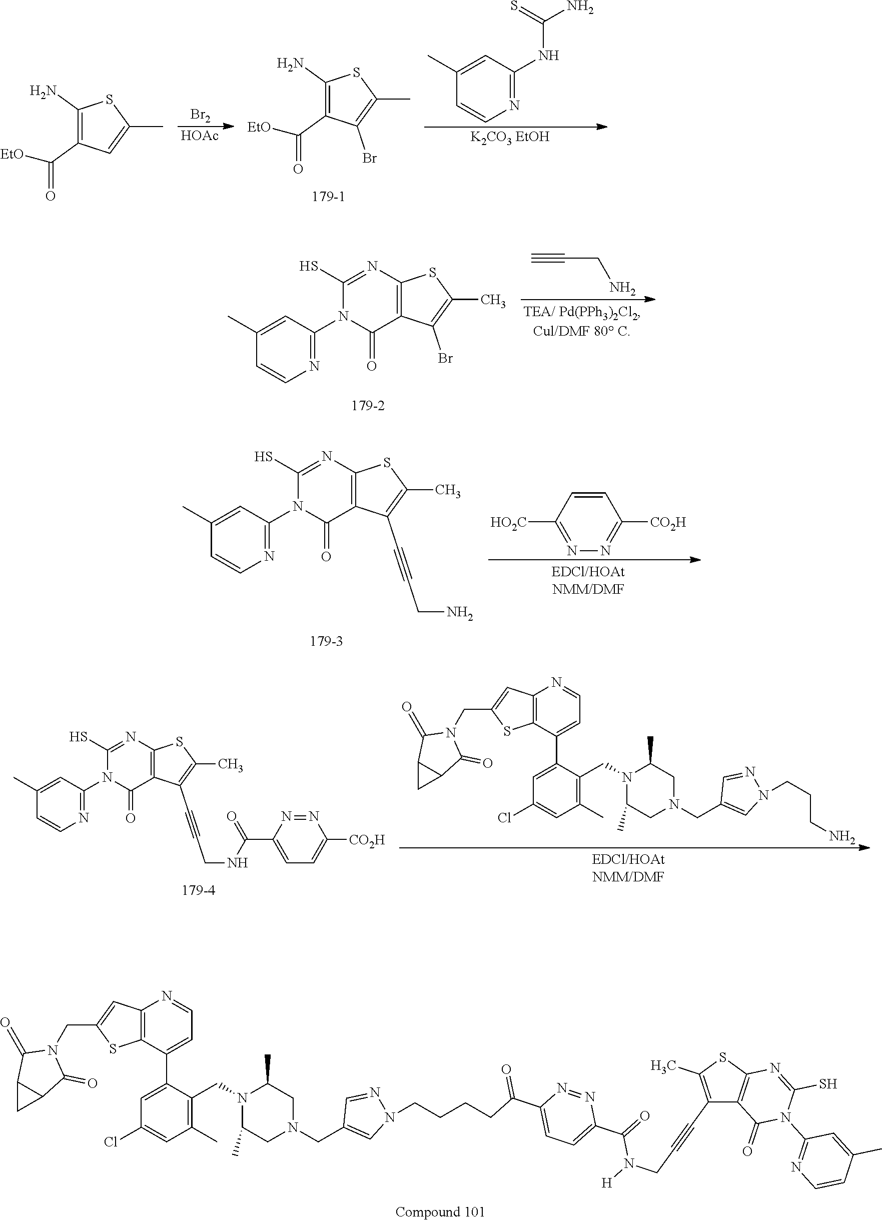

### Example 180: Synthesis of (R)—N3-(3-(3-((4-hydroxy-1-(3-phenylbutanoyl)piperidin-4-yl)methyl)-4-oxo-3,4-dihydroquinazolin-7-yl)prop-2-yn-1-yl)-N6-(3-(2-mercapto-6-methyl-3-(4-methylpyridin-2-yl)-4-oxo-3,4-dihydrothieno[2,3-d]pyrimidin-5-yl)prop-2-yn-1-yl)pyridazine-3,6-dicarboxamide Compound 102

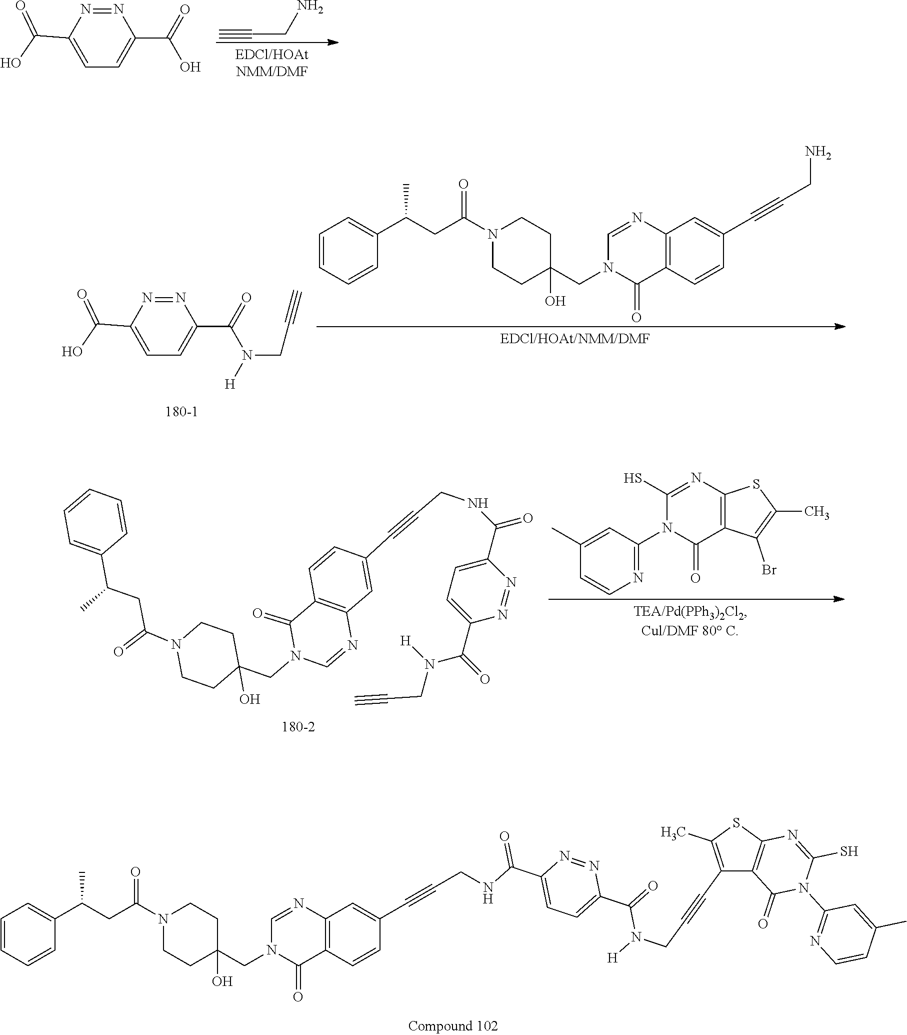

### Example 181: Synthesis of (R)—N3-(3-(3-((4-hydroxy-1-(3-phenylbutanoyl)piperidin-4-yl)methyl)-4-oxo-3,4-dihydroquinazolin-7-yl)propyl)-N6-(3-(2-mercapto-6-methyl-3-(4-methylpyridin-2-yl)-4-oxo-3,4-dihydrothieno[2,3-d]pyrimidin-5-yl)propyl)pyridazine-3,6-dicarboxamide Compound 103

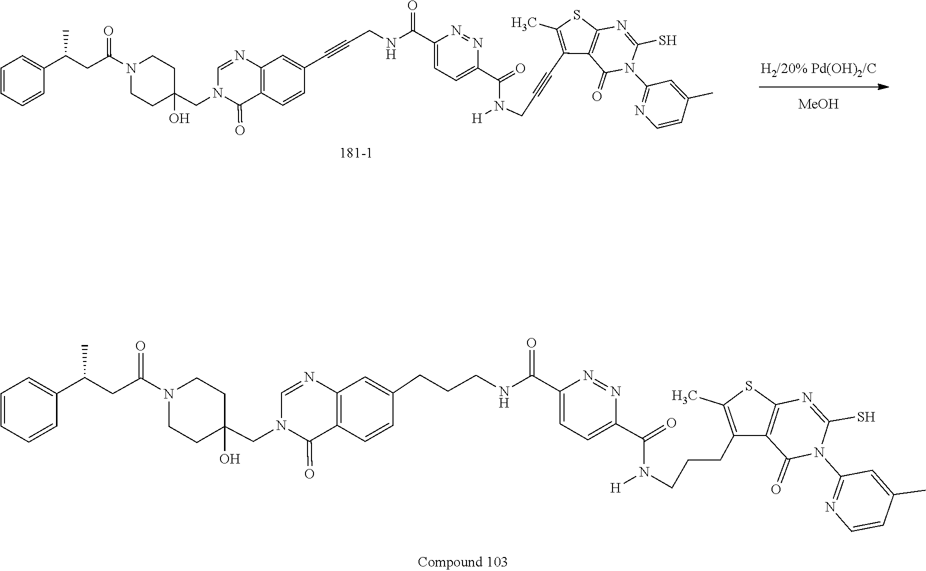 [SMILES: "MeOH" ; Cc1ccnc(-n2c(S)nc3sc(C)c(C#CCNC(=O)c4ccc(C(=O)NCC#Cc5ccc6c(=O)n(CC7(O)CCN(C(=O)C[C@@H](C)c8ccccc8)CC7)cnc6c5)nn4)c3c2=O)c1 ; Cc1ccnc(-n2c(S)nc3sc(C)c(CCCNC(=O)c4ccc(C(=O)NCCCc5ccc6c(=O)n(CC7(O)CCN(C(=O)C[C@@H](C)c8ccccc8)CC7)cnc6c5)nn4)c3c2=O)c1]

### Example 182: Synthesis of N3-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propyl)-N6-(3-(2-mercapto-6-methyl-3-(4-methylpyridin-2-yl)-4-oxo-3,4-dihydrothieno[2,3-d]pyrimidin-5-yl)propyl)pyridazine-3,6-dicarboxamide Compound 104

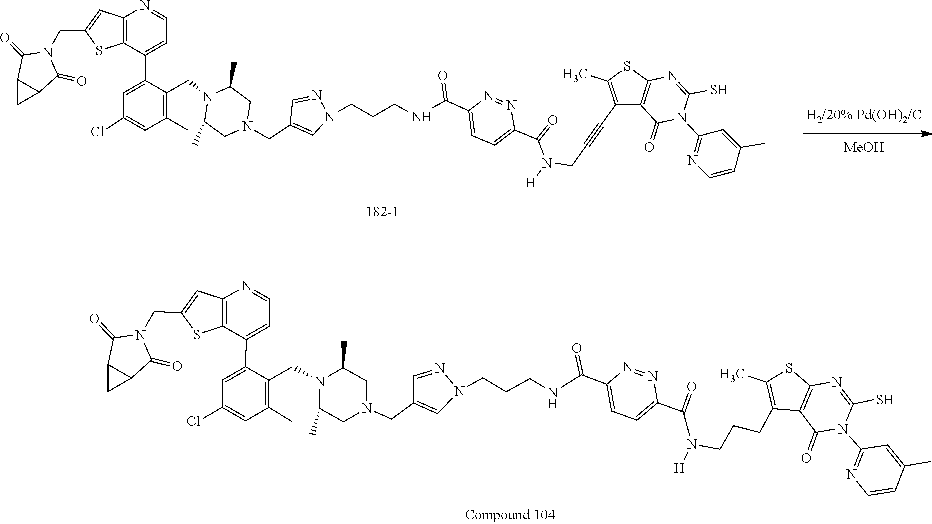

### Example 183: Synthesis of 5-(3-(((4-ethyl-3,5-bis(4-hydroxyphenyl)pyridin-2-yl)methyl)amino)prop-1-yn-1-yl)-2-mercapto-6-methyl-3-(4-methylpyridin-2-yl)thieno[2,3-d]pyrimidin-4(3H)-one Compound 105

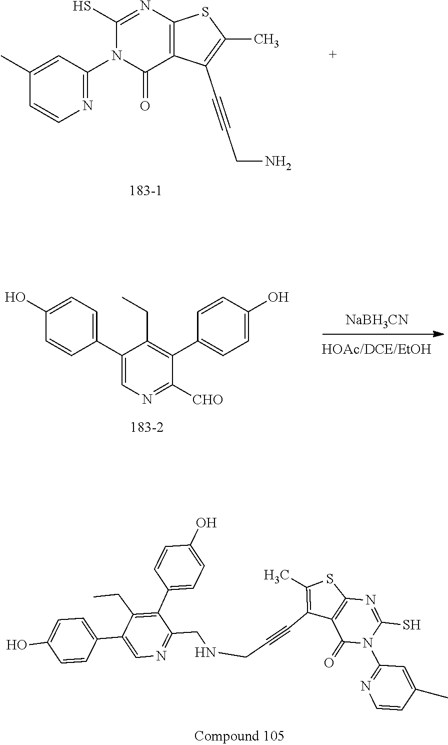 [SMILES: Cc1ccnc(-n2c(S)nc3sc(C)c(C#CCN)c3c2=O)c1 ; CCc1c(-c2ccc(O)cc2)cnc(C=O)c1-c1ccc(O)cc1 ; "NaBH3CN" ; CCc1c(-c2ccc(O)cc2)cnc(CNCC#Cc2c(C)sc3nc(S)n(-c4cc(C)ccn4)c(=O)c23)c1-c1ccc(O)cc1]

### Example 184: Synthesis of 5-(3-(((4-ethyl-3,5-bis(4-hydroxyphenyl)pyridin-2-yl)methyl)amino)propyl)-2-mercapto-6-methyl-3-(4-methylpyridin-2-yl)thieno[2,3-d]pyrimidin-4(3H)-one Compound 106

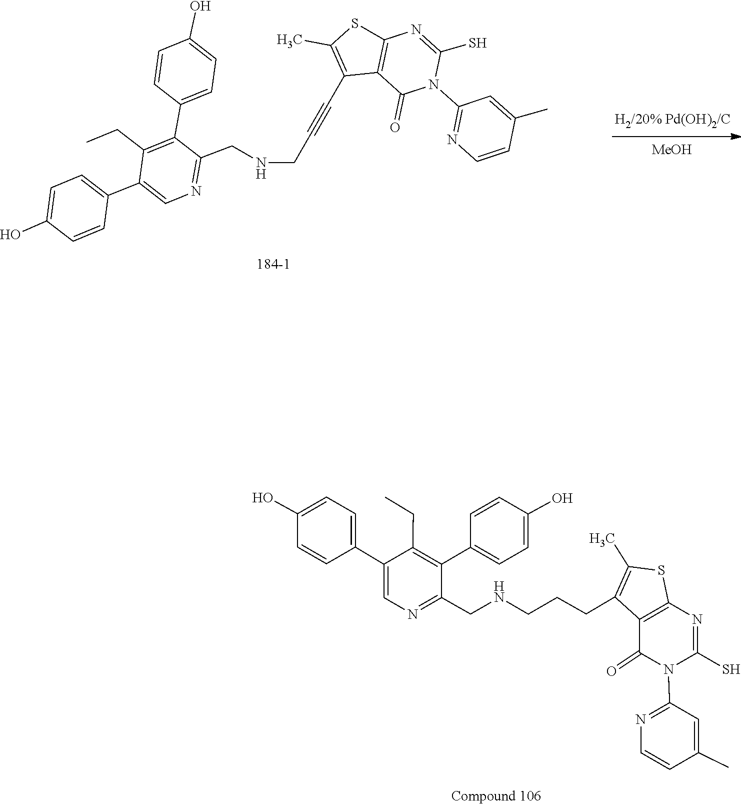 [SMILES: "MeOH" ; CCc1c(-c2ccc(O)cc2)cnc(CNCC#Cc2c(C)sc3nc(S)n(-c4cc(C)ccn4)c(=O)c23)c1-c1ccc(O)cc1 ; CCc1c(-c2ccc(O)cc2)cnc(CNCCCc2c(C)sc3nc(S)n(-c4cc(C)ccn4)c(=O)c23)c1-c1ccc(O)cc1]

### Example 185: Synthesis of (R)-5-(3-(((1-(4-(3-((4-hydroxy-1-(3-phenylbutanoyl)piperidin-4-yl)methyl)-4-oxo-3,4-dihydropyrrolo[2,1-f][1,2,4]triazin-7-yl)benzyl)-1H-pyrazol-4-yl)methyl)amino)prop-1-yn-1-yl)-2-mercapto-6-methyl-3-(4-methylpyridin-2-yl)thieno[2,3-d]pyrimidin-4(3H)-one Compound 107

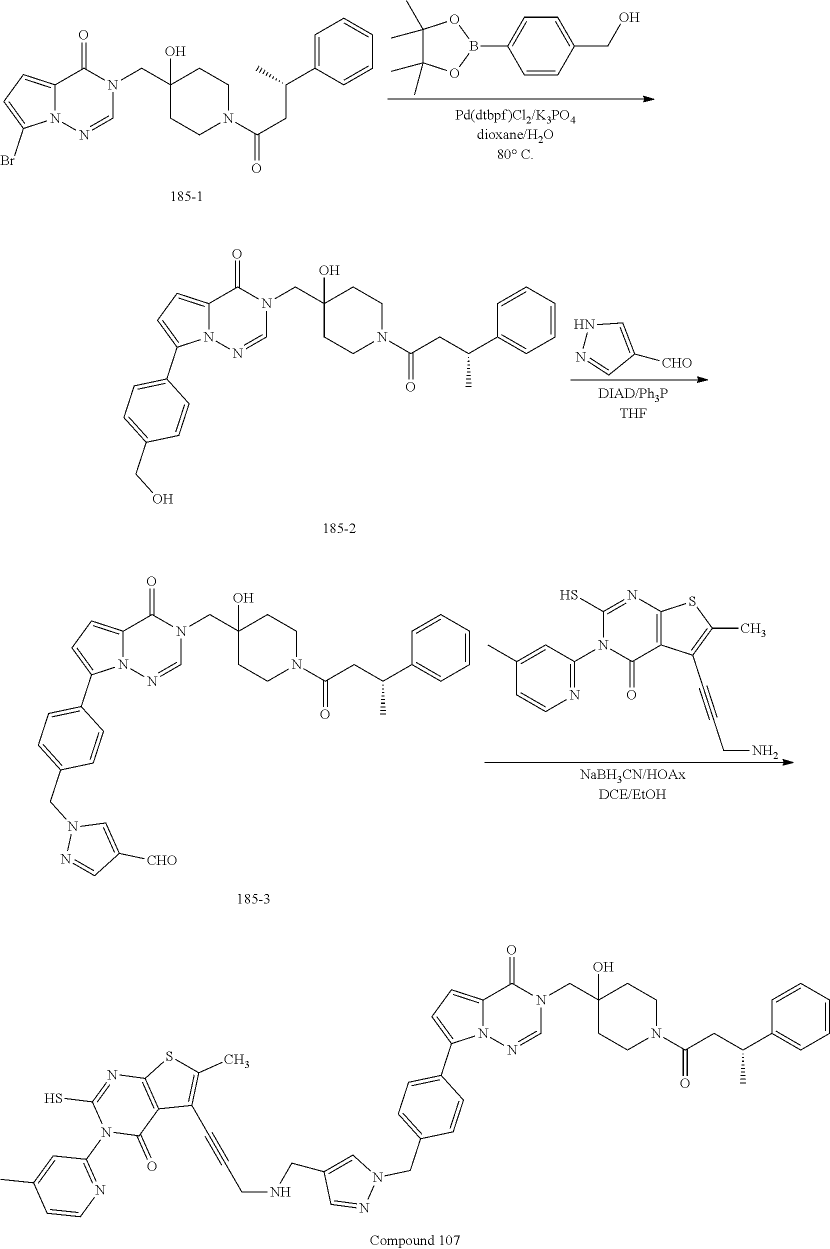

### Example 186: Synthesis of (R)-5-(3-(((1-(4-(3-((4-hydroxy-1-(3-phenylbutanoyl)piperidin-4-yl)methyl)-4-oxo-3,4-dihydropyrrolo[2,1-f][1,2,4]triazin-7-yl)benzyl)-1H-pyrazol-4-yl)methyl)amino)propyl)-2-mercapto-6-methyl-3-(4-methylpyridin-2-yl)thieno[2,3-d]pyrimidin-4(3H)-one Compound 108

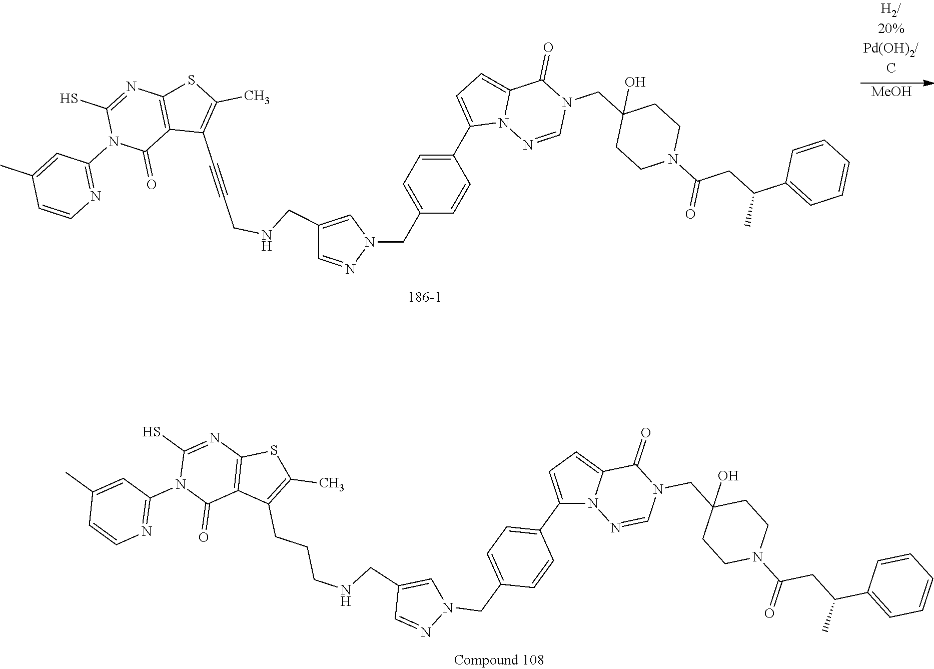 [SMILES: "MeOH" ; Cc1ccnc(-n2c(S)nc3sc(C)c(C#CCNCc4cnn(Cc5ccc(-c6ccc7c(=O)n(CC8(O)CCN(C(=O)C[C@@H](C)c9ccccc9)CC8)cnn67)cc5)c4)c3c2=O)c1 ; Cc1ccnc(-n2c(S)nc3sc(C)c(CCCNCc4cnn(Cc5ccc(-c6ccc7c(=O)n(CC8(O)CCN(C(=O)C[C@@H](C)c9ccccc9)CC8)cnn67)cc5)c4)c3c2=O)c1]

### Example 187: Synthesis of N-(1-((2R)-3-(3-(4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)propoxy)-2-hydroxypropyl)-6-fluoro-2-(1-hydroxy-2-methylpropan-2-yl)-1H-indol-5-yl)-1-(2,2-difluorobenzo[d][1,3]dioxol-5-yl)cyclopropane-1-carboxamide Compound 109

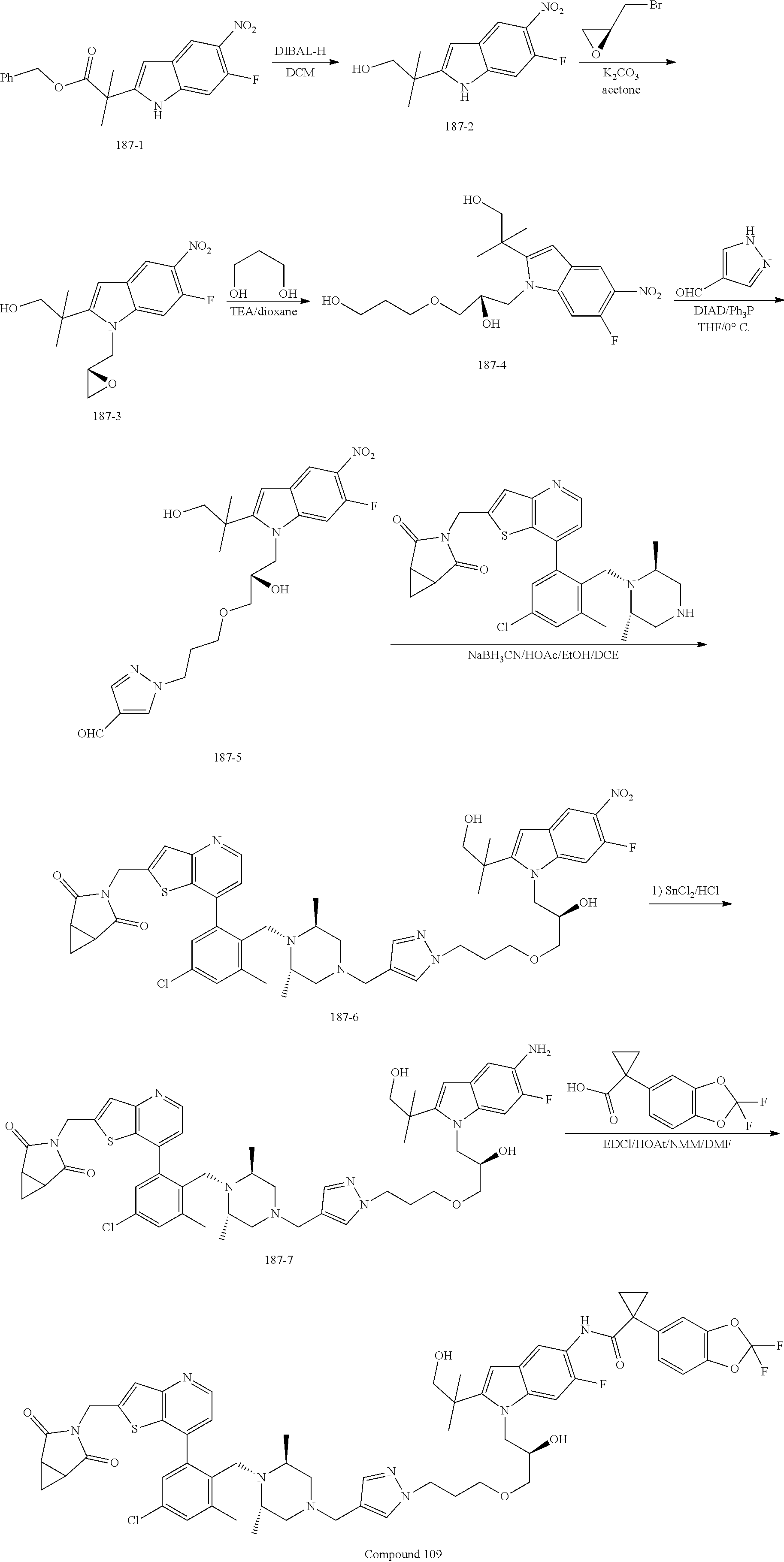

### Example 188: Synthesis of (R)—N-(1-(3-(4-(((6-amino-4-ethyl-3,5-bis(4-hydroxyphenyl)pyridin-2-yl)methoxy)methyl)-1H-1,2,3-triazol-1-yl)-2-hydroxypropyl)-6-fluoro-2-(1-hydroxy-2-methylpropan-2-yl)-1H-indol-5-yl)-1-(2,2-difluorobenzo[d][1,3]dioxol-5-yl)cyclopropane-1-carboxamide Compound 110

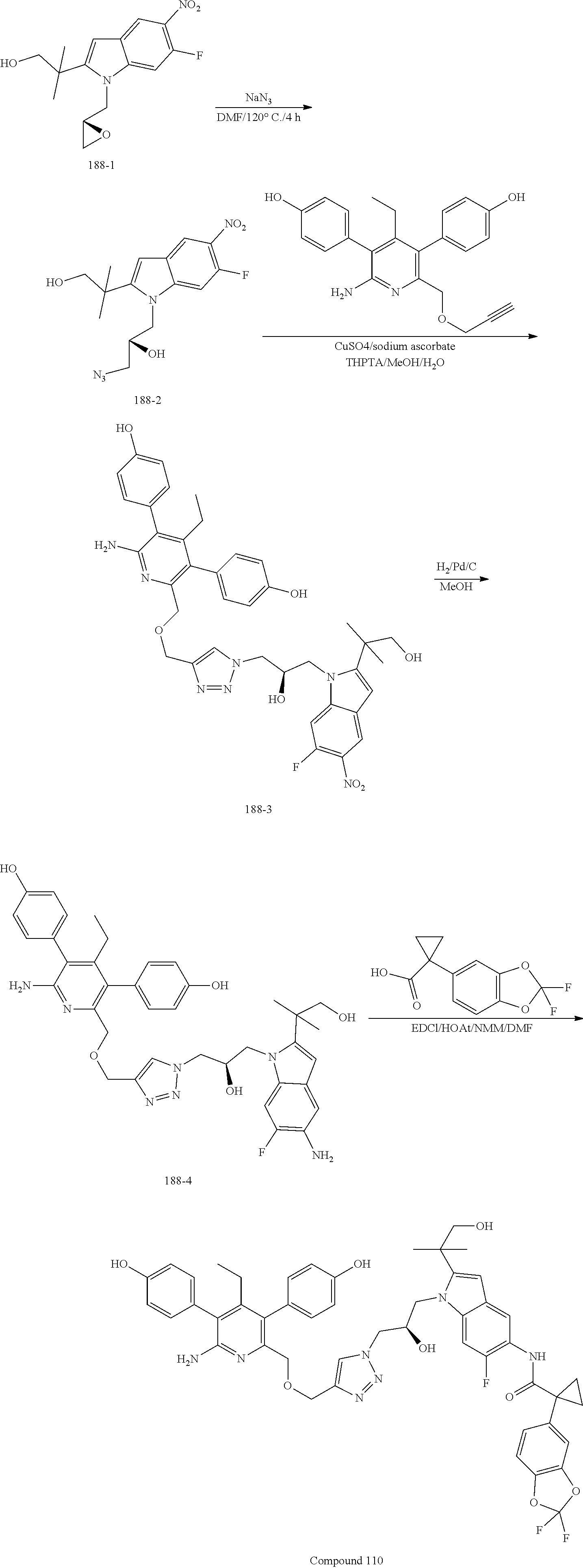

### Example 189: Synthesis of (R)—N-(1-(3-(3-(4-(((6-amino-4-ethyl-3,5-bis(4-hydroxyphenyl)pyridin-2-yl)methoxy)methyl)-1H-1,2,3-triazol-1-yl)propoxy)-2-hydroxypropyl)-6-fluoro-2-(1-hydroxy-2-methylpropan-2-yl)-1H-indol-5-yl)-1-(2,2-difluorobenzo[d][1,3]dioxol-5-yl)cyclopropane-1-carboxamide Compound 111

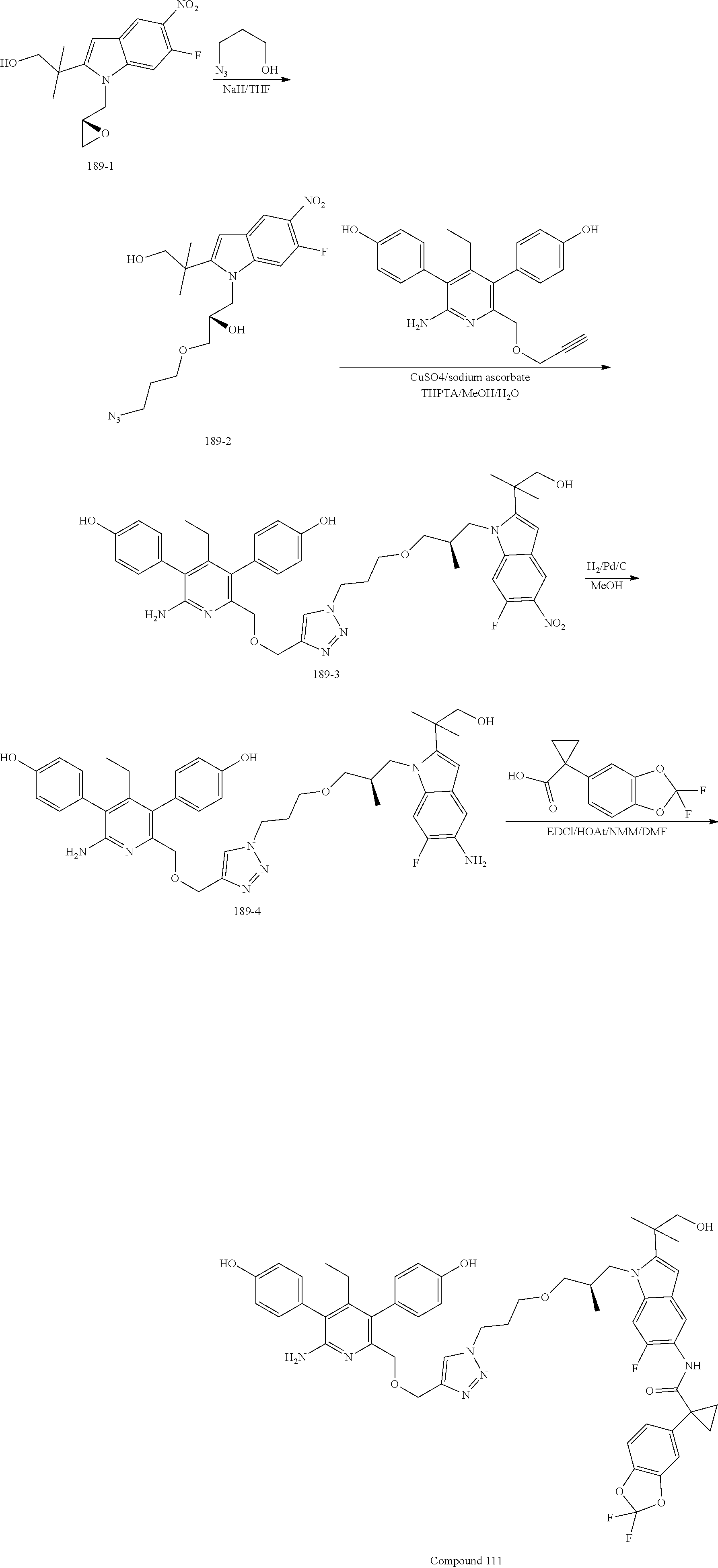

### Example 190: Synthesis of 14-(5-(1-(2,2-difluorobenzo[d][1,3]dioxol-5-yl)cyclopropane-1-carboxamido)-1-((R)-2,3-dihydroxypropyl)-6-fluoro-1H-indol-2-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-14-methyl-3,6,9,12-tetraoxapentadecanamide Compound 112

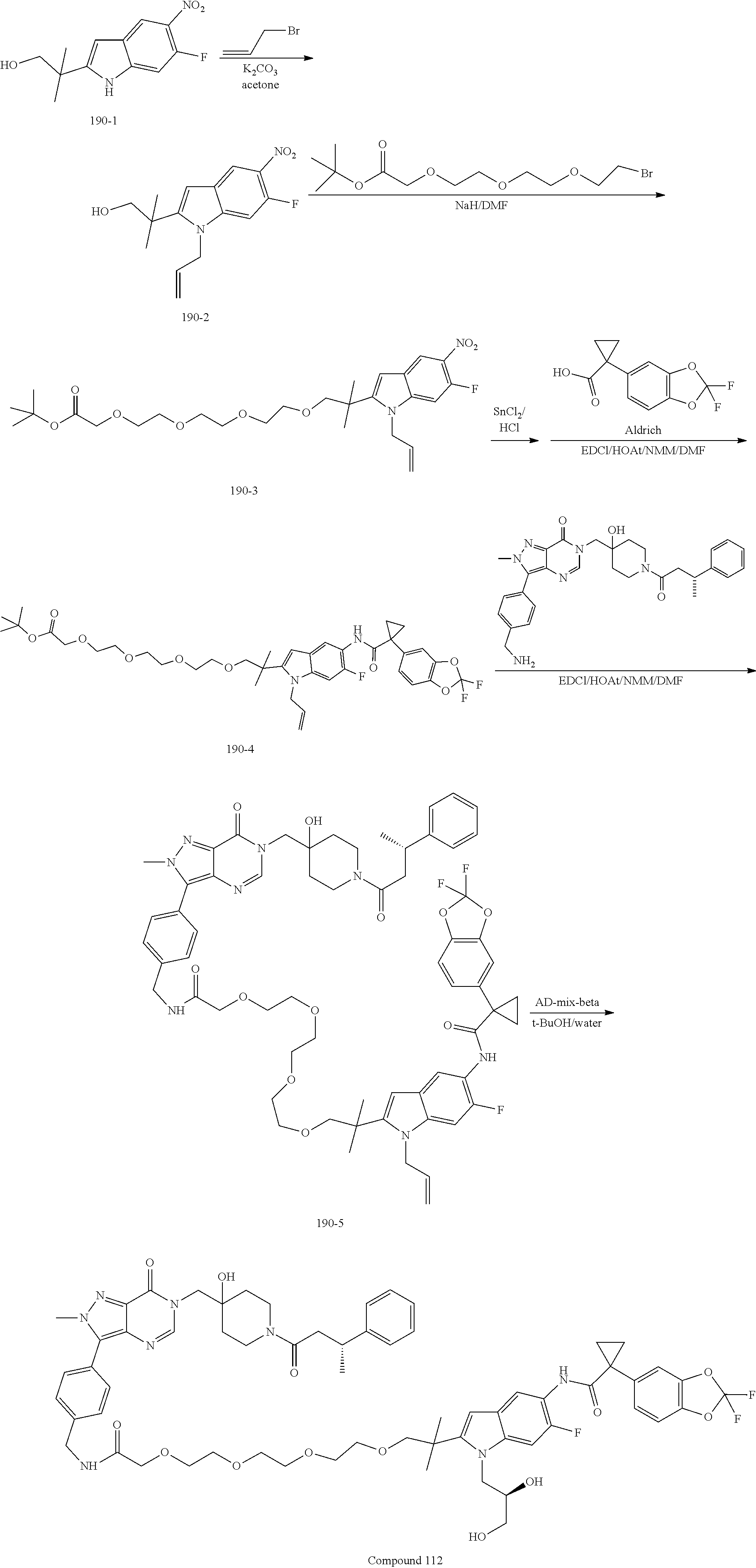

### Example 191: Synthesis of 1-(2,2-difluorobenzo[d][1,3]dioxol-5-yl)-N-(1-((R)-2,3-dihydroxypropyl)-6-fluoro-2-(1-((5-((6-((4-(((3-(3-((4-hydroxy-1-((R)-3-phenylbutanoyl)piperidin-4-yl)methyl)-4-oxo-3,4-dihydroquinazolin-7-yl)prop-2-yn-1-yl)amino)methyl)-1H-pyrazol-1-yl)methyl)pyridazin-3-yl)methoxy)pentyl)oxy)-2-methylpropan-2-yl)-1H-indol-5-yl)cyclopropane-1-carboxamide Compound 113

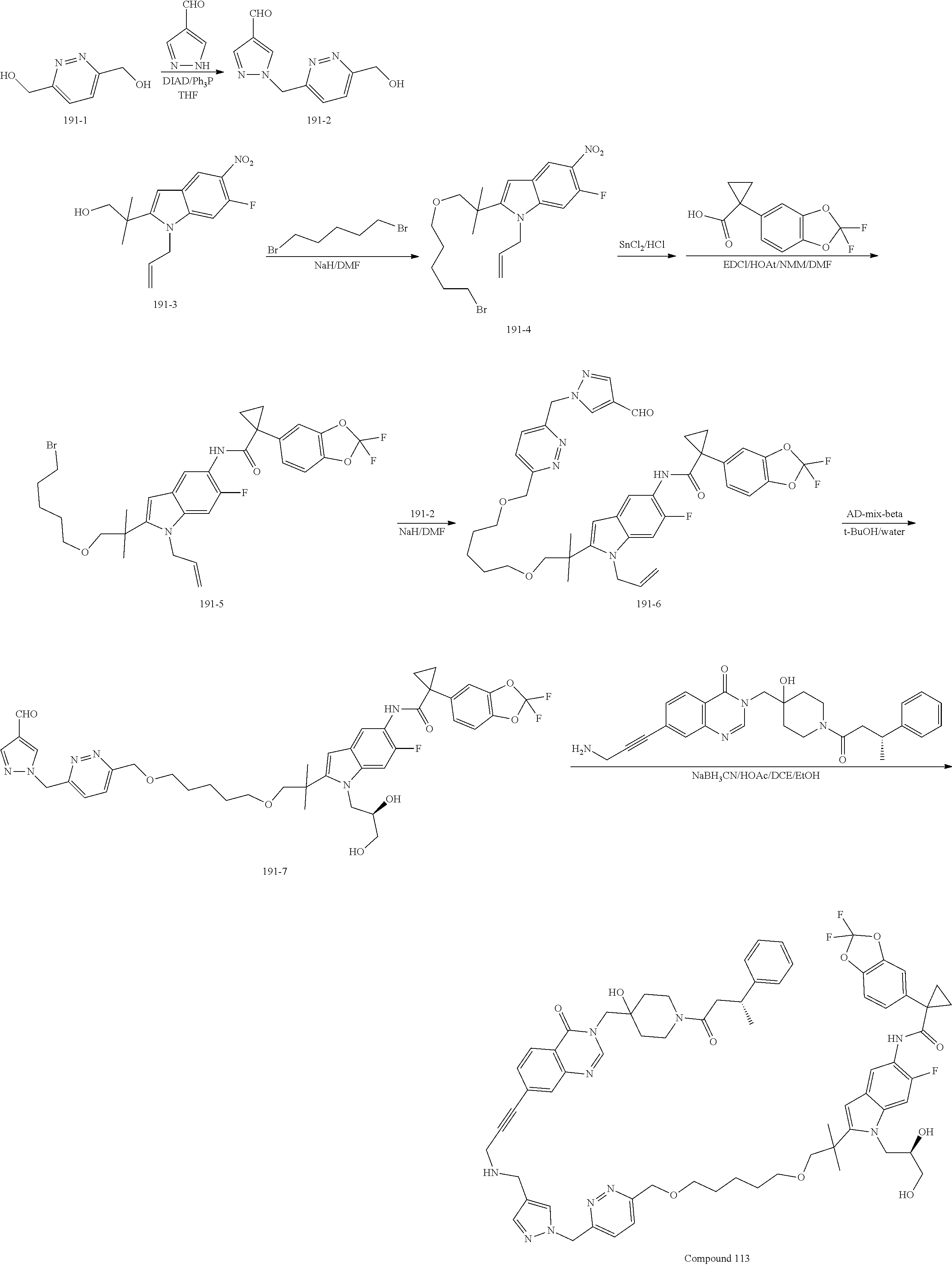

### Example 192: Synthesis of 1-((6-(((5-(2-(5-(1-(2,2-difluorobenzo[d][1,3]dioxol-5-yl)cyclopropane-1-carboxamido)-1-((R)-2,3-dihydroxypropyl)-6-fluoro-1H-indol-2-yl)-2-methylpropoxy)pentyl)oxy)methyl)pyridazin-3-yl)methyl)-N-(3-(3-((4-hydroxy-1-((R)-3-phenylbutanoyl)piperidin-4-yl)methyl)-4-oxo-3,4-dihydroquinazolin-7-yl)prop-2-yn-1-yl)-1H-pyrazole-4-carboxamide Compound 114

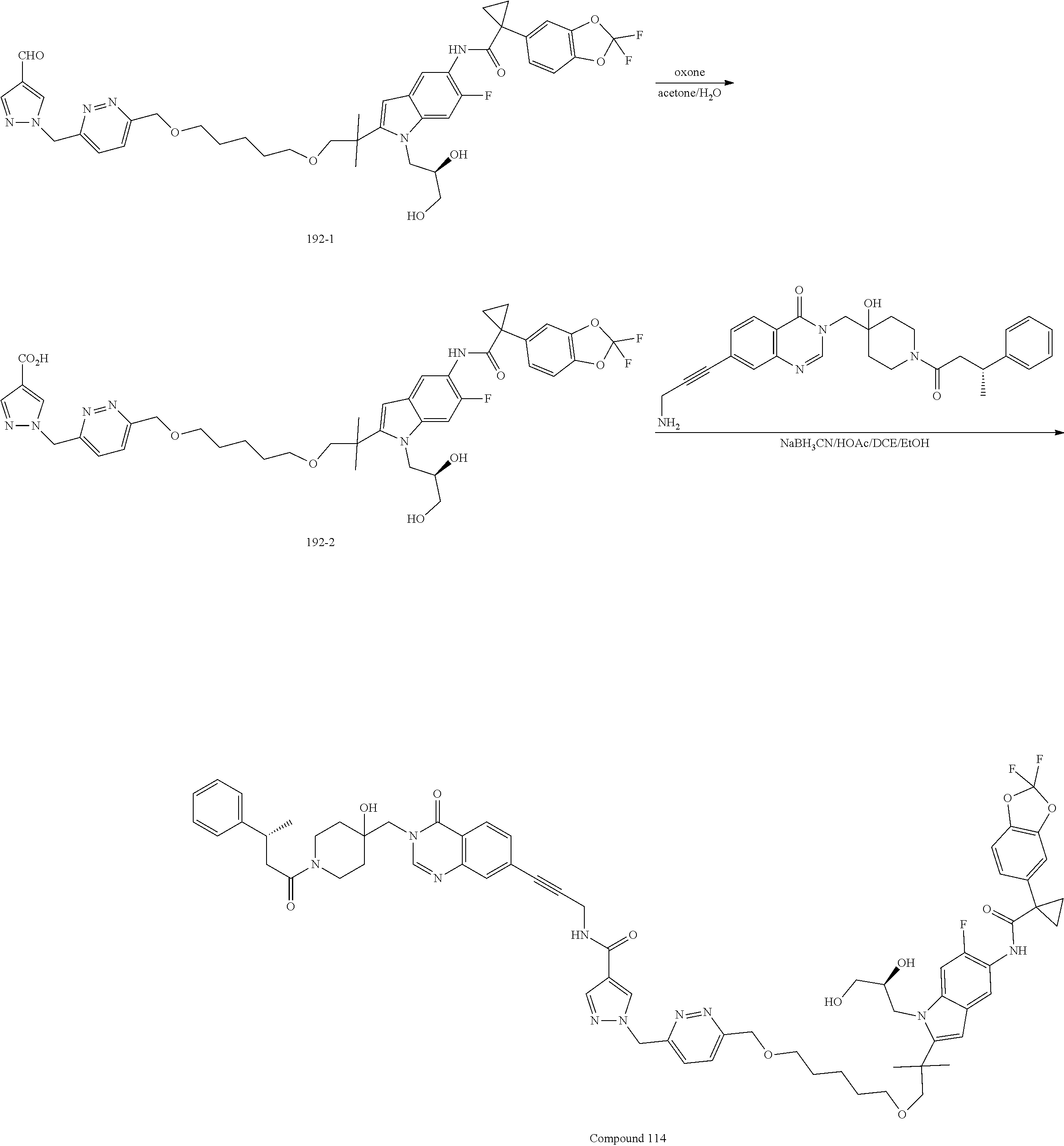 [SMILES: CC(C)(COCCCCCOCc1ccc(Cn2cc(C=O)cn2)nn1)c1cc2cc(NC(=O)C3(c4ccc5c(c4)OC(F)(F)O5)CC3)c(F)cc2n1C[C@@H](O)CO ; CC(C)(COCCCCCOCc1ccc(Cn2cc(C(=O)O)cn2)nn1)c1cc2cc(NC(=O)C3(c4ccc5c(c4)OC(F)(F)O5)CC3)c(F)cc2n1C[C@@H](O)CO ; C[C@H](CC(=O)N1CCC(O)(Cn2cnc3cc(C#CCN)ccc3c2=O)CC1)c1ccccc1 ; C[C@H](CC(=O)N1CCC(O)(Cn2cnc3cc(C#CCNC(=O)c4cnn(Cc5ccc(COCCCCCOCC(C)(C)c6cc7cc(NC(=O)C8(c9ccc%10c(c9)OC(F)(F)O%10)CC8)c(F)cc7n6C[C@H](O)CO)nn5)c4)ccc3c2=O)CC1)c1ccccc1]

### Example 193: Synthesis of N-(2-(1-((5-((6-((4-((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazine-1-carbonyl)-1H-pyrazol-1-yl)methyl)pyridazin-3-yl)methoxy)pentyl)oxy)-2-methylpropan-2-yl)-1-((S)-2,3-dihydroxypropyl)-6-fluoro-1H-indol-5-yl)-1-(2,2-difluorobenzo[d][1,3]dioxol-5-yl)cyclopropane-1-carboxamide Compound 115

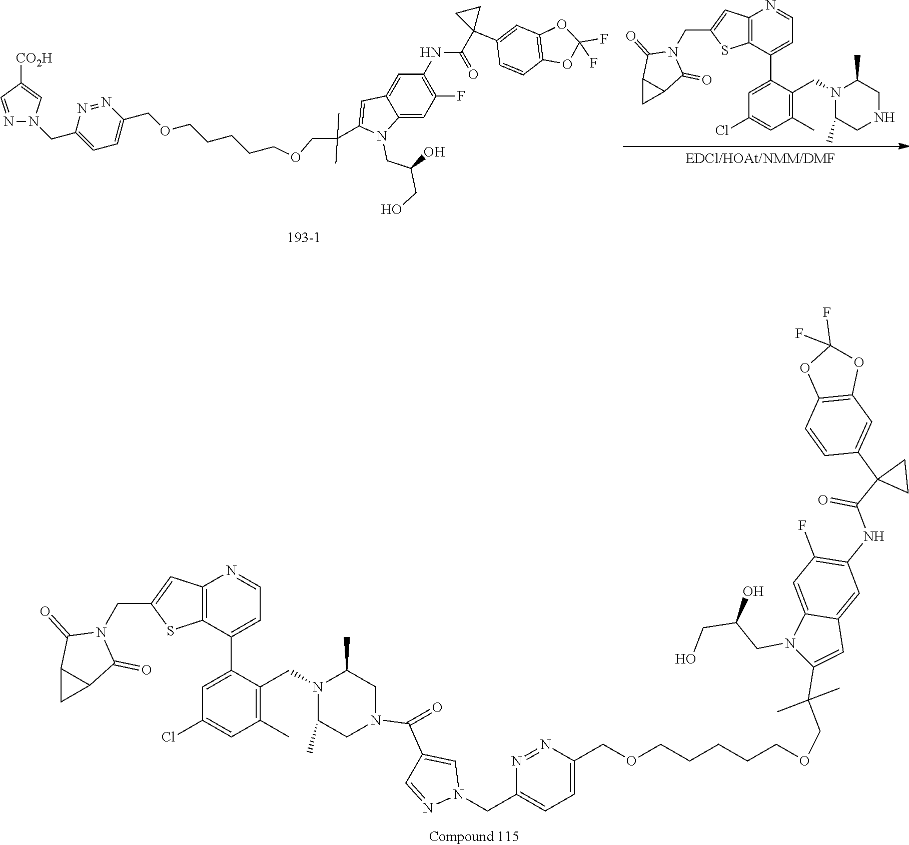

### Example 194: Synthesis of N-(2-(1-((5-((6-((4-(((3S,5S)-4-(4-chloro-2-(2-((2,4-dioxo-3-azabicyclo[3.1.0]hexan-3-yl)methyl)thieno[3,2-b]pyridin-7-yl)-6-methylbenzyl)-3,5-dimethylpiperazin-1-yl)methyl)-1H-pyrazol-1-yl)methyl)pyridazin-3-yl)methoxy)pentyl)oxy)-2-methylpropan-2-yl)-1-((S)-2,3-dihydroxypropyl)-6-fluoro-1H-indol-5-yl)-1-(2,2-difluorobenzo[d][1,3]dioxol-5-yl)cyclopropane-1-carboxamide Compound 115

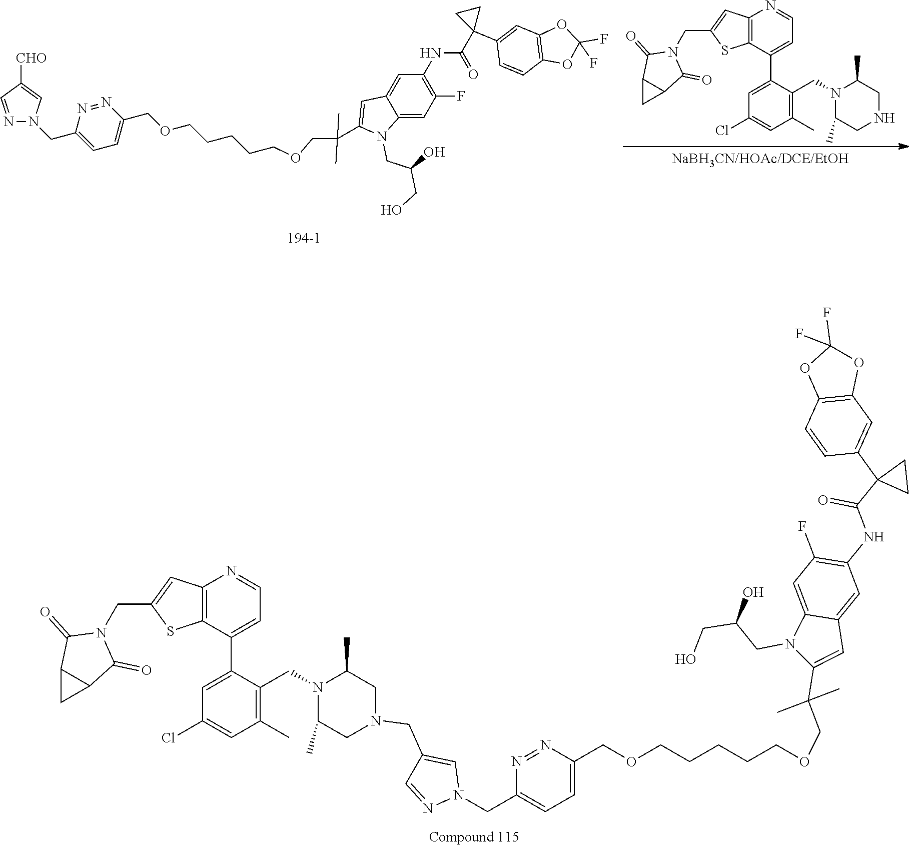

### Example 195. Synthesis of (5-((4-(4-(5-(4-((4-(4-(((6-(5-methyl-2,4-dioxo-3,4-dihydropyrimidin-1(2H)-yl)hexyl)oxy)diphenylmethyl)benzoyl)piperazin-1-yl)methyl)piperidin-1-yl)pyrazine-2-carboxamido)phenyl)piperidin-1-yl)sulfonyl)picolinoyl)glycine (Compound 116)

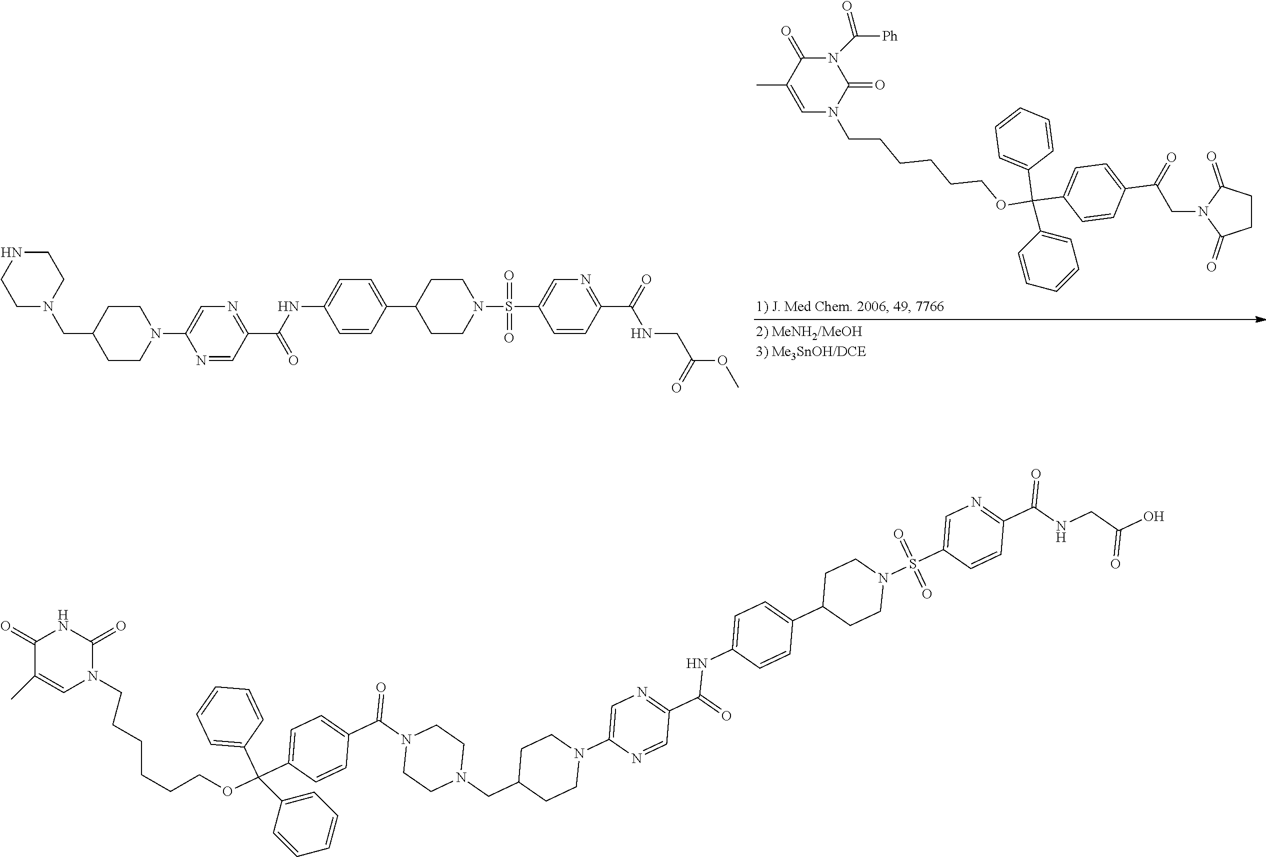 [SMILES: COC(=O)CNC(=O)c1ccc(S(=O)(=O)N2CCC(c3ccc(NC(=O)c4cnc(N5CCC(CN6CCNCC6)CC5)cn4)cc3)CC2)cn1 ; Cc1cn(CCCCCCOC(c2ccccc2)(c2ccccc2)c2ccc(C(=O)CN3C(=O)CCC3=O)cc2)c(=O)n(C(=O)c2ccccc2)c1=O ; Cc1cn(CCCCCCOC(c2ccccc2)(c2ccccc2)c2ccc(C(=O)N3CCN(CC4CCN(c5cnc(C(=O)Nc6ccc(C7CCN(S(=O)(=O)c8ccc(C(=O)NCC(=O)O)nc8)CC7)cc6)cn5)CC4)CC3)cc2)c(=O)[nH]c1=O]

### Example 196. Synthesis of (5-((4-(4-(7-(4-(((6-(5-methyl-2,4-dioxo-3,4-dihydropyrimidin-1(2H)-yl)hexyl)oxy)diphenylmethyl)benzamido)heptanamido)phenyl)piperidin-1-yl)sulfonyl)picolinoyl)glycine (Compound 117)

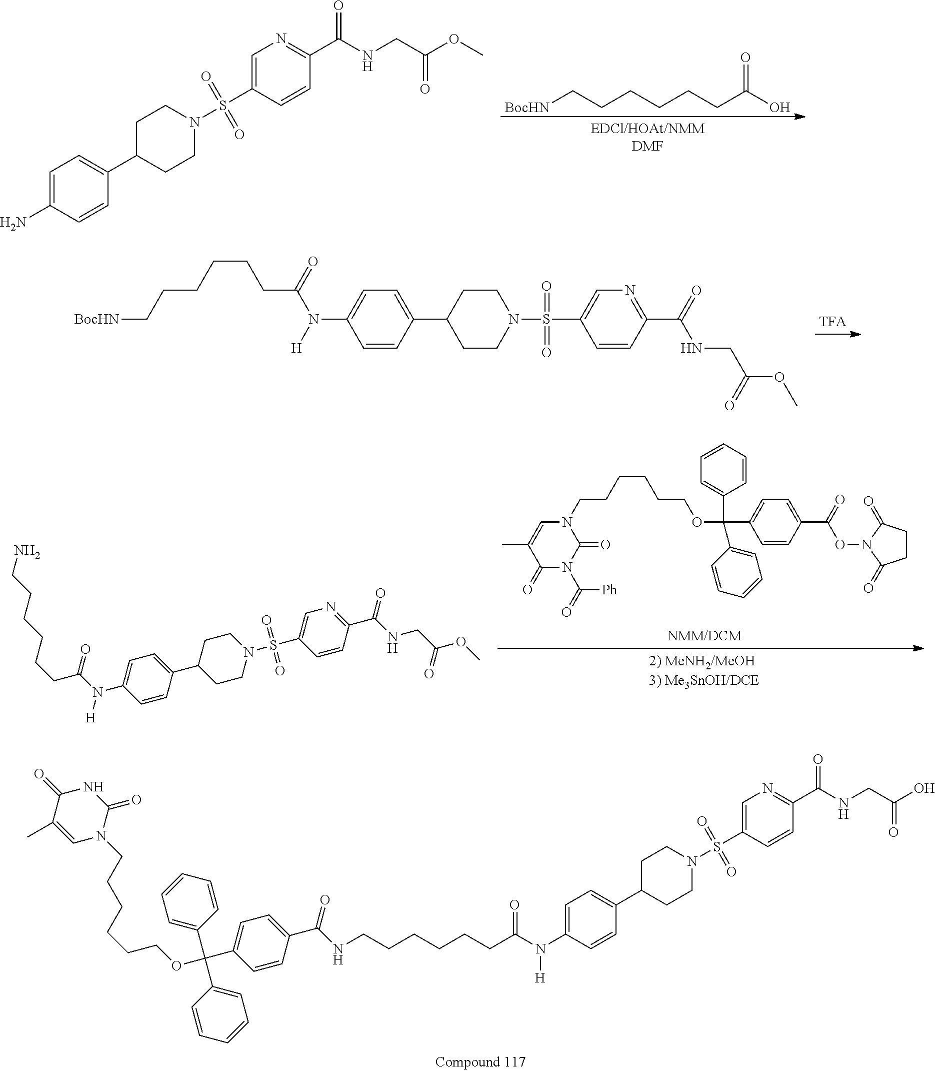

### Example 197. Synthesis of [[5-[2-[[9-ethyl-7-(4-methyl-2-thienyl)carbazol-3-yl]methylamino]ethyl]-N-[6-(2-thienylsulfonylamino)-1,3-benzothiazol-2-yl]-4,6,7,8-tetrahydropyrazolo[1,5-a][1,4]diazepine-2-carboxamide]] (Compound 118)

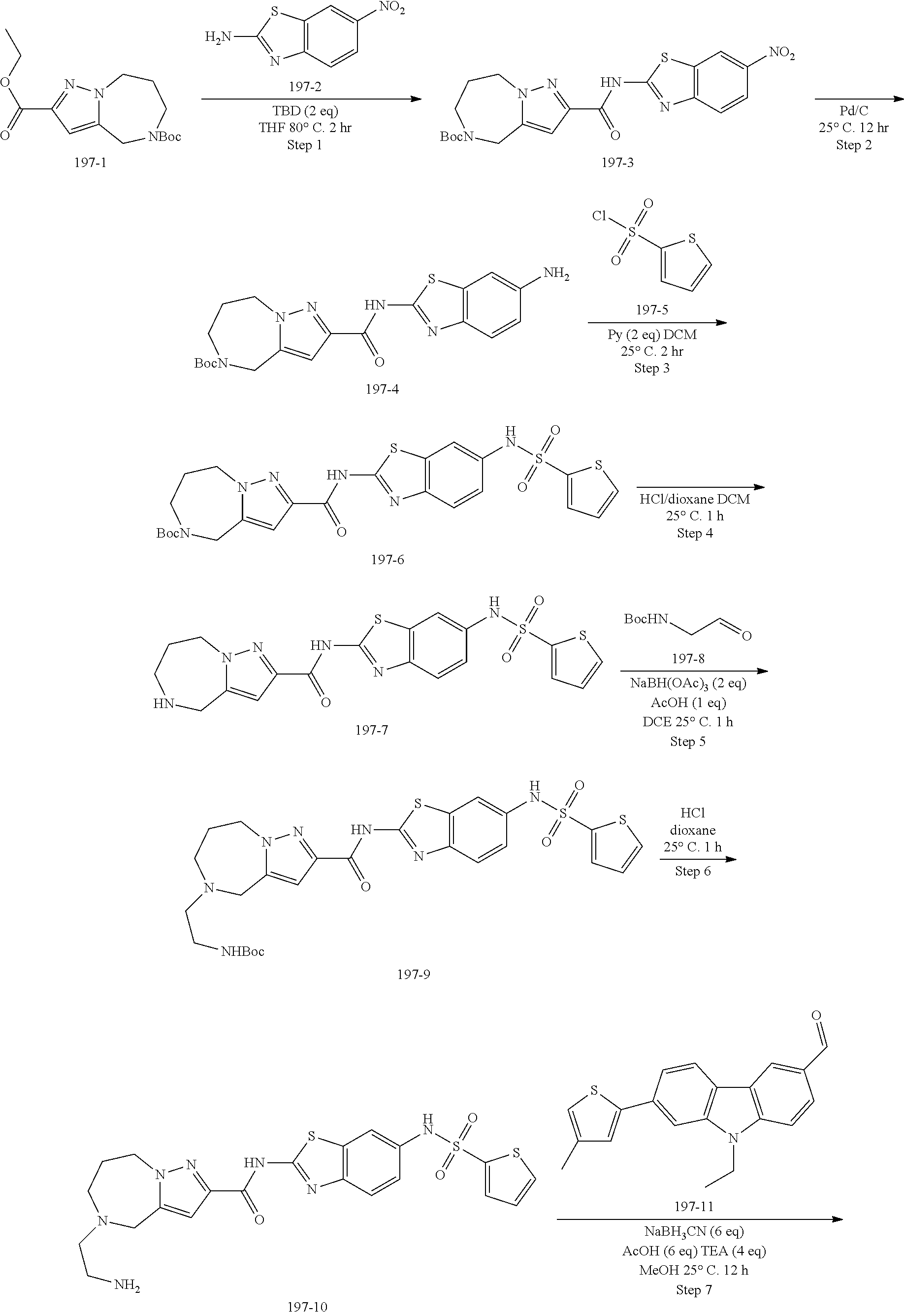

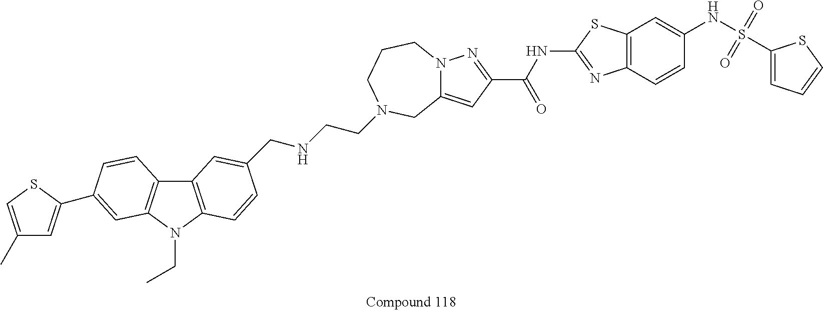 [SMILES: CCn1c2ccc(CNCCN3CCCn4nc(C(=O)Nc5nc6ccc(NS(=O)(=O)c7cccs7)cc6s5)cc4C3)cc2c2ccc(-c3cc(C)cs3)cc21]

### Step 1: Synthesis of Intermediate 197-3

Detailed Synthetic Procedure: To a solution of intermediate 197-1 (5.26 g, 26.96 mmol, 1.5 eq) and intermediate 197-2 (5.56 g, 17.97 mmol, 1 eq) in THE (60 mL) was added 3,4,6,7,8,9-hexahydro-2H-pyrimido[1,2-a]pyrimidine (5.00 g, 35.95 mmol, 2 eq). The mixture was stirred at 80° C. for 2 hrs. LCMS showed desired mass was detected. TLC (PE/EA=1:1) indicated 197-2 (Rt=0.8) was remained and product one new spot (Rt=0.2) formed. The reaction was clean according to TLC. The mixture was poured into water (20 mL) and extracted with EA (100 mL×3). The combined organic layers were dried over Na2SO4, filtered and concentrated under reduced pressure to give a residue. The residue was purified by flash silica gel chromatography (eluent of 0-100% Ethyl acetate/Petroleum ether; gradient 150 mL/min), which was concentrated under reduced pressure to give Intermediate 1-3 (8 g, 16.46 mmol, 91.57% yield, 94.319% purity) as yellow solid which was confirmed by LCMS and HNMR.

### Mass Found

LCMS: Retention time: 0.581 min, (M+H)=459.0

LCMS: Retention time: 0.588 min, (M+H)=458.9

### NMR Data

¹H NMR (400 MHz, DMSO-d6) δ=12.93-12.59 (m, 1H), 9.06 (d, J=2.4 Hz, 1H), 8.29-8.27 (m, 1H), 7.90 (d, J=8.8 Hz, 1H), 7.05-6.95 (m, 1H), 4.53 (s, 2H), 3.67 (s, 2H), 3.33-3.16 (m, 2H), 1.91-1.78 (m, 2H), 1.35 (s, 9H).

### Step 2: Synthesis of Intermediate 197-4

To a solution of Intermediate 197-3 (6.1 g, 13.30 mmol, 1 eq) in EtOH (46 mL) and H₂O (15 mL) was added Fe (2.23 g, 39.91 mmol, 3 eq) and NH₄Cl (3.56 g, 66.52 mmol, 5 eq). The mixture was stirred at 80° C. for 0.5 hr. LCMS showed the desired mass was detected. TLC (DCM/MeOH=10:1) indicated 197-3 (Rt=0.2) was consumed and product one new spot (Rt=0.8) formed. The reaction was clean according to TLC. The mixture was pour into water (20 mL) and extracted with EA (100 mL×3). The combined organic layers were dried over Na₂SO₄, filtered and concentrated under reduced pressure to give a residue. The residue was purified by flash silica gel chromatography (eluent of 0˜100% DCM/MeOH @ 150 mL/min), which was concentrated under reduced pressure to give 197-4 (5.55 g, 12.95 mmol, 97.35% yield) as yellow solid which was confirmed by LCMS and HNMR.

### Mass Found

LCMS: Retention time: 0.351 min, (M+H)=429.2

LCMS: Retention time: 0.424 min, (M+H)=429.0

### NMR Data

1H NMR (400 MHz, DMSO-d6) δ=12.15-11.53 (m, 1H), 7.43 (d, J=7.6 Hz, 1H), 7.05-6.89 (m, 2H), 6.72 (d, J=7.2 Hz, 1H), 5.19 (s, 2H), 4.51 (s, 2H), 3.66 (s, 2H), 1.79 (s, 2H), 1.35 (s, 9H).

### Step 3: Synthesis of Intermediate 197-6

Detailed Synthetic Procedure: To a solution of intermediate 197-4 (4.53 g, 10.57 mmol, 1 eq), Py (1.67 g, 21.14 mmol, 1.71 mL, 2 eq) in DCM (45 mL), and added intermediate 197-5 (1.93 g, 10.57 mmol, 1 eq), then the mixture was stirred at 0° C. for 0.5 hr. Then the mixture was stirred at 25° C. for 2 hrs. LCMS showed desired mass was detected. TLC (DCM/MeOH=10:1) indicated 197-4 (Rt=0.5) was consumed and product one new spot (Rt=0.4) formed. The reaction was clean according to TLC. The mixture was pour into water (50 mL) and extracted with EA (100 mL*3). The combined organic layers were dried over Na2SO4, filtered and concentrated under reduced pressure to give a residue. The residue was purified by flash silica gel chromatography eluent of 0-100% DCM/MeOH @ 150 mL/min), the eluent was concentrated under reduced pressure to give intermediate 197-6 (7.3 g, crude) as purple solid which was confirmed by LCMS and HNMR.

### Mass Found

LCMS: Retention time: 0.566 min, (M+H)=574.9

LCMS: Retention time: 0.510 min, (M+H)=575.5

### NMR Data

¹H NMR (400 MHz, DMSO-d6) δ=12.31 (s, 1H), 10.47 (s, 1H), 7.88-7.87 (m, 1H), 7.75 (d, J=1.6 Hz, 1H), 7.67 (d, J=8.8 Hz, 1H), 7.54-7.53 (m, 1H), 7.20-7.19 (m, 1H), 7.11-7.09 (m, 1H), 7.02-6.89 (m, 1H), 4.53 (s, 2H), 4.11 (q, J=5.2 Hz, 2H), 3.67 (s, 2H), 1.80 (s, 2H), 1.35 (s, 9H).

### Step 4: Synthesis of Intermediate 197-7

To a solution of intermediate 197-6 (6.3 g, 10.96 mmol, 1 eq) in HCl/dioxane (45 mL, 4M) and DCM (20 mL). The mixture was stirred at 25° C. for 1 hr. LCMS showed desired mass was detected. The mixture was concentrated under reduced pressure to give a residue. The residue without purification, and it was concentrated under reduced pressure to give intermediate 1-7 (3.5 g, 7.37 mmol, 67.28% yield) as white solid which was confirmed by LCMS and HNMR.

### Mass Found

LCMS: Retention time: 0.389 min, (M+H)=474.8

LCMS: Retention time: 0.318 min, (M+H)=475.1

### NMR Data

¹H NMR (400 MHz, DMSO-d6) δ=7.89-7.85 (m, 1H), 7.75 (d, J=2.0 Hz, 1H), 7.66 (d, J=8.8 Hz, 1H), 7.53-7.52 (m, 1H), 7.21-7.20 (m, 1H), 7.13-7.08 (m, 2H), 4.60-4.51 (m, 2H), 4.37 (s, 2H), 3.35 (d, J=5.2 Hz, 2H), 2.02 (s, 2H).

### Step 5: Synthesis of Intermediate 197-9

To a solution of intermediate 197-7 (200 mg, 421.43 umol, 1 eq) and intermediate 197-8 (268.33 mg, 1.69 mmol, 4 eq) in DCE (2 mL) was added AcOH (25.31 mg, 421.43 umol, 24.10 uL, 1 eq). The mixture was stirred at 25° C. for 0.25 hr, which was added sodium triacetoxyboranuide (178.63 mg, 842.85 umol, 2 eq). Then the mixture was stirred at 25° C. for 1 hr. LCMS showed desired mass was detected. The mixture was pour into water (5 mL) and extracted with EA (5 mL*3). The combined organic layers were dried over Na2SO4, filtered and concentrated under reduced pressure to give a residue. The crude product was purified by reversed-phase HPLC (0.1% FA modifier in ACN/H₂O), the eluent was lyophilizated to give intermediate 197-9 (120 mg, 189.56 umol, 44.98% yield, 97.584% purity) as yellow solid which was confirmed by LCMS and HNMR.

### Mass Found

LCMS: Retention time: 0.389 min, (M+H)=618.2

LCMS: Retention time: 0.384 min, (M+H)=618.1

### NMR Data

¹H NMR (400 MHz, DMSO-d6) δ=7.85 (d, J=4.8 Hz, 1H), 7.71 (s, 1H), 7.63 (d, J=9.2 Hz, 1H), 7.51 (d, J=3.2 Hz, 1H), 7.16 (d, J=8.4 Hz, 1H), 7.09 (t, J=4.4 Hz, 1H), 6.90 (s, 1H), 6.68-6.60 (m, 2H), 4.45-4.40 (m, 2H), 3.91 (s, 2H), 3.06 (s, 2H), 3.02 (d, J=6.0 Hz, 2H), 2.95-2.90 (m, 3H), 2.67 (s, 2H), 1.38 (s, 9H).

### Step 6: Synthesis of Intermediate 197-10

To a solution of intermediate 197-9 (100 mg, 161.87 umol, 1 eq) in DCM (0.3 mL) and HCl/dioxane (0.7 mL, 4M). The mixture was stirred at 25° C. for 0.5 hr. LCMS showed desired mass was detected. The mixture was filtered and concentrated under reduced pressure to give a residue. The residue without purification, and it was concentrated under reduced pressure to give intermediate 197-10 (150 mg, crude, HCl) as white solid.

### Mass Found

LCMS: Retention time: 0.325 min, (M+H)=518.0

### Step 7: Synthesis of Compound 1

To a solution of intermediate 197-10 (20 mg, 36.09 umol, 1 eq, HCl) in MeOH (0.4 mL) was added TEA (14.61 mg, 144.38 umol, 20.10 uL, 4 eq). The mixture was stirred at 25° C. 0.5 hr. Then NaBH₃CN (13.61 mg, 216.56 umol, 6 eq), AcOH (13.01 mg, 216.56 umol, 12.39 uL, 6 eq) and intermediate 197-11 (11.53 mg, 36.09 umol, 1 eq) was added into the mixture, which was stirred at 25° C. 12 hrs. LCMS showed the desired mass was detected. The mixture was filtered and concentrated under reduced pressure to give a residue. The residue was purified by prep-HPLC (column: Phenomenex luna C18 150*25 mm*10 um; mobile phase: [water(FA)-ACN];B %: 30%-60%, 10 min), which was lyophilizated to give Compound 118 (4.5 mg, 5.17 umol, 14.32% yield, 94.318% purity) as brown solid which was confirmed by LCMS and HNMR.

### Mass Found

LCMS: Retention time: 0.505 min, (M+H)=821.2,

LCMS: Retention time: 0.501 min, (M+H)=821.2,

### NMR Data

¹H NMR (400 MHz, DMSO-d6) δ=8.21 (s, 1H), 8.17-7.94 (m, 2H), 7.83 (s, 2H), 7.71 (s, 1H), 7.66-7.61 (m, 1H), 7.57 (d, J=8.0 Hz, 1H), 7.51-7.40 (m, 4H), 7.21-7.14 (m, 1H), 7.13-7.05 (m, 2H), 6.91 (s, 1H), 4.53-4.39 (m, 4H), 4.01 (s, 2H), 3.93 (s, 2H), 3.07 (s, 2H), 2.80 (s, 2H), 2.53 (s, 2H), 2.26 (s, 3H), 1.77 (d, J=1.6 Hz, 2H), 1.36-1.28 (m, 3H).

### Example 198. Synthesis of [[5-[3-[[9-ethyl-7-(4-methyl-2-thienyl)carbazol-3-yl]methylamino]propyl]-N-[6-(2-thienylsulfonylamino)-1,3-benzothiazol-2-yl]-4,6,7,8-tetrahydropyrazolo[1,5-a][1,4]diazepine-2-carboxamide]] (Compound 119)

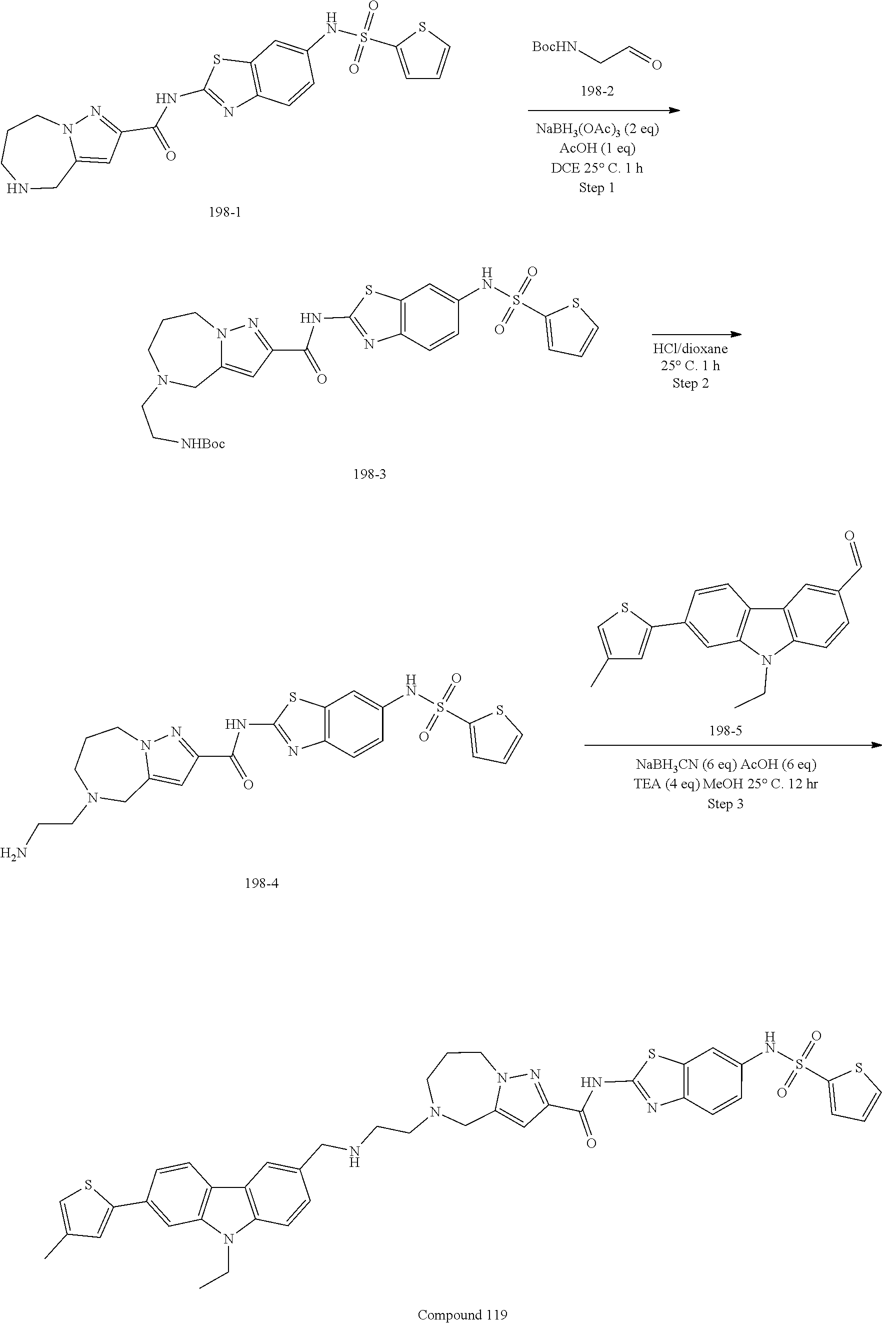 [SMILES: O=C(Nc1nc2ccc(NS(=O)(=O)c3cccs3)cc2s1)c1cc2n(n1)CCCNC2 ; CC(C)(C)OC(=O)NCC=O ; CC(C)(C)OC(=O)NCCN1CCCn2nc(C(=O)Nc3nc4ccc(NS(=O)(=O)c5cccs5)cc4s3)cc2C1 ; NCCN1CCCn2nc(C(=O)Nc3nc4ccc(NS(=O)(=O)c5cccs5)cc4s3)cc2C1 ; CCn1c2ccc(C=O)cc2c2ccc(-c3cc(C)cs3)cc21 ; CCn1c2ccc(CNCCN3CCCn4nc(C(=O)Nc5nc6ccc(NS(=O)(=O)c7cccs7)cc6s5)cc4C3)cc2c2ccc(-c3cc(C)cs3)cc21]

### Step 1: Synthesis of Intermediate 1-3

To a solution of intermediate 1-1 (200 mg, 421.43 umol, 1 eq) and intermediate 1-2 (291.98 mg, 1.69 mmol, 4 eq) in DCE (2 mL) was added AcOH (25.31 mg, 421.43 umol, 24.10 uL, 1 eq). The mixture was stirred at 25° C. for 0.25 hr, which was added sodium triacetoxyboranuide (178.63 mg, 842.85 umol, 2 eq). Then the mixture was stirred at 25° C. for 1 hr. LCMS showed desired mass was detected. TLC (DCM/MeOH=10:1) indicated 198-1 (Rt=0.5) was consumed and product one new spot (Rt=0.4) formed. The reaction was clean according to TLC. The mixture was poured into water (5 mL) and extracted with EA (5 mL*3). The combined organic layers were dried over Na2SO4, filtered and concentrated under reduced pressure to give a residue. The residue was purified by flash silica gel chromatography (eluent of 0-100% DCM/MeOH @ 80 mL/min), and was concentrated under reduced pressure to give intermediate 198-3 (180 mg, 284.29 umol, 67.46% yield, 99.784% purity) as yellow solid confirmed by LCMS and HNMR.

### Mass Found

LCMS: Retention time: 0.387 min, (M+H)=632.1

LCMS: Retention time: 0.393 min, (M+H)=632.2

### NMR Data

¹H NMR (400 MHz, DMSO-d6) δ=7.85 (d, J=4.0 Hz, 1H), 7.71 (s, 1H), 7.64 (d, J=8.0 Hz, 1H), 7.51 (d, J=1.6 Hz, 1H), 7.17 (d, J=8.8 Hz, 1H), 7.11-7.07 (m, 1H), 6.89 (s, 1H), 6.73 (s, 1H), 4.43 (d, J=3.2 Hz, 2H), 3.88 (s, 2H), 3.11-2.99 (m, 3H), 2.90 (d, J=6.0 Hz, 3H), 1.75 (s, 3H), 1.55-1.46 (m, 3H), 1.35 (s, 9H).

### Step 2: Synthesis of Intermediate 198-4

To a solution of intermediate 198-3 (100 mg, 158.28 umol, 1 eq) in DCM (0.3 mL) and HCl/dioxane (0.7 mL, 4M). The mixture was stirred at 25° C. for 1 hr. The LCMS showed desired mass was detected. The mixture was filtered and concentrated under reduced pressure to give a residue. The residue without purification, and it was concentrated under reduced pressure to give intermediate 198-4 (150 mg, crude, HCl) as yellow solid.

### Mass Found

LCMS: Retention time: 0.298 min, (M+H)=532.1

### Step 3: Synthesis of Compound 119

To a solution of intermediate 198-4 (70 mg, 123.21 umol, 1 eq, HCl) in MeOH (1 mL) was added TEA (49.87 mg, 492.84 umol, 68.60 uL, 4 eq). The mixture was stirred at 25° C. 0.5 hr. Then NaBH3CN (46.46 mg, 739.26 umol, 6 eq), AcOH (44.39 mg, 739.26 umol, 42.28 uL, 6 eq) and intermediate 198-5 (78.71 mg, 246.42 umol, 2 eq) was added into the mixture, which was stirred at 25° C. 12 hrs. LCMS showed the desired mass was detected. The mixture was concentrated under reduced pressure to give a residue. The residue was purified by prep-HPLC (column: Phenomenex luna C18 150*25 mm*10 um;mobile phase: [water(FA)-ACN];B %: 25%-55%, 10 min), eluent was lyophilizated to give Compound 119 (19 mg, 22.46 umol, 18.23% yield, 98.725% purity) as off-white solid confirmed by LCMS and HNMR.

### Mass Found

LCMS: Retention time: 0.495 min, (M+H)=835.3

LCMS: Retention time: 0.510 min, (M+H)=835.3

### NMR Data

¹H NMR (400 MHz, DMSO-d6) δ=8.26 (s, 1H), 8.13-8.05 (m, 2H), 7.85-7.79 (m, 2H), 7.69 (s, 1H), 7.61 (d, J=8.4 Hz, 1H), 7.58-7.53 (m, 1H), 7.51-7.39 (m, 4H), 7.22-7.01 (m, 3H), 6.91 (s, 1H), 4.52-4.39 (m, 4H), 4.02 (s, 2H), 3.89 (s, 2H), 3.05 (s, 2H), 2.72 (t, J=6.4 Hz, 2H), 2.38 (s, 2H), 2.27 (s, 3H), 1.79-1.63 (m, 4H), 1.29 (t, J=6.8 Hz, 3H).

### Example 199. Synthesis of [[5-[4-[[9-ethyl-7-(4-methyl-2-thienyl)carbazol-3-yl]methylamino] butyl]-N-[6-(2-thienylsulfonylamino)-1,3-benzothiazol-2-yl]-4,6,7,8-tetrahydropyrazolo[1,5-a][1,4]diazepine-2-carboxamide]] (Compound 120)

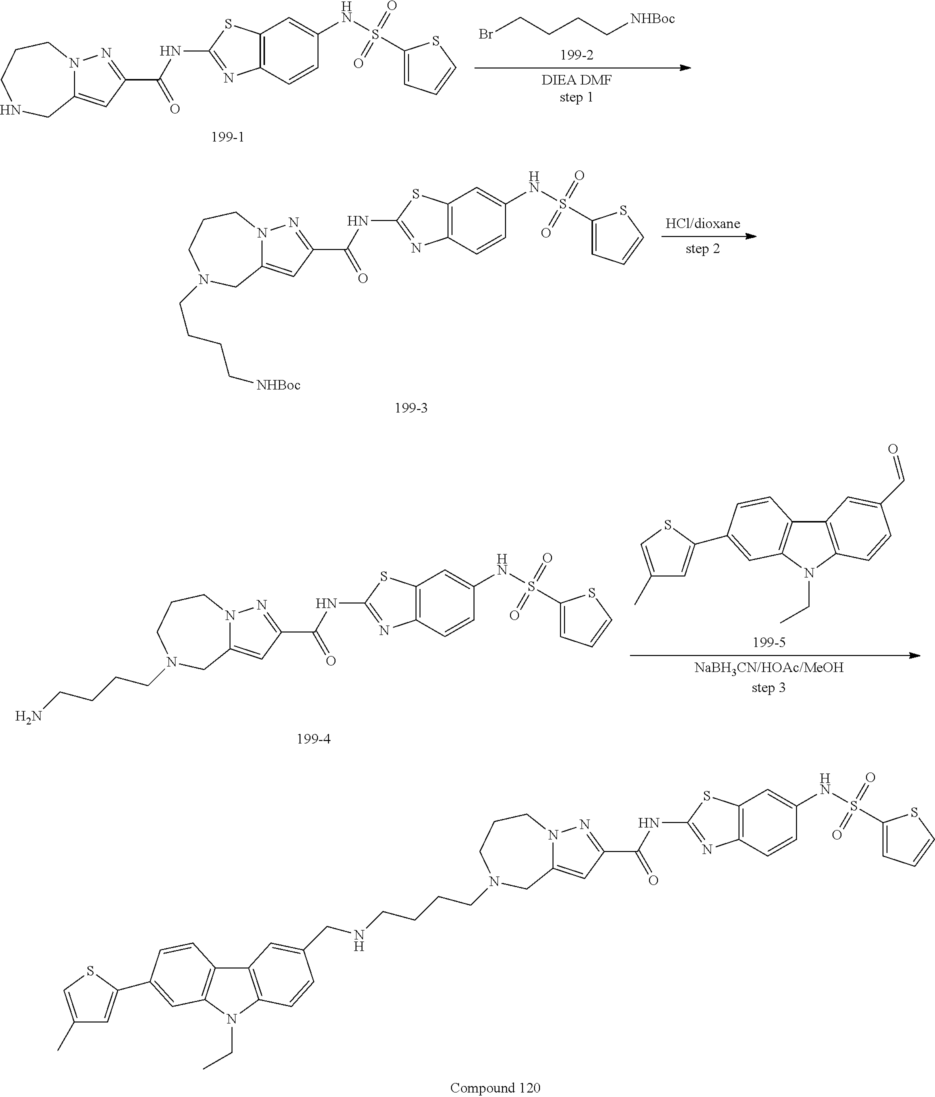

### Step 1: Synthesis of Intermediate 199-3

To a solution of intermediate 199-1 (100 mg, 210.71 umol, 1 eq) and intermediate 199-2 (159.39 mg, 632.14 umol, 129.59 uL, 3 eq) in DMF (1 mL) was added TEA (63.97 mg, 632.14 umol, 87.99 uL, 3 eq). The mixture was stirred at 25° C. for 2 hrs. LCMS showed 199-1 was consumed and one major peak with desired mass was detected. The reaction was quenched with H2O (5 mL). The mixture was extract with EA (10 mL*3). The combined organic layers dried over Na2SO4, filtered and concentrated under reduced pressure to give a residue. The residue was purified by flash silica gel chromatography (eluent of 0˜100% DCM/MeOH @ 60 mL/min, DCM:MeOH=10:1, Rt=0.5) and the eluent was concentrated under reduced pressure to give product. 199-3 (120 mg, 152.37 umol, 72.31% yield, 82% purity) as a white solid and confirmed by LCMS.

### Mass Found

LCMS: Retention time: 0.382 min, (M+H)=646.3

LCMS: Retention time: 0.395 min, (M+H)=646.3

### Step 2: Synthesis of Intermediate 198-4

To a solution of intermediate 198-3 (120 mg, 185.81 umol, 1 eq) in dioxane (1 mL) was added HCl/dioxane (4 M, 46.45 uL, 1 eq). The mixture was stirred at 25° C. for 0.5 hr. LCMS showed 198-3 was consumed and 78% of desired mass was detected. The mixture was filtered and concentrated under reduced pressure to give a residue. The crude product was used into the next step without further purification. Intermediate 198-4 (130 mg, crude, HCl) was obtained as a white solid.

### Mass Found

LCMS: Retention time: 0.302 min, (M+H)=546.1

### Step 3: Synthesis of Compound 120

To a solution of 199-4 (120 mg, 206.13 umol, 1 eq, HCl) in EtOH (1 mL) was added TEA (83.43 mg, 824.51 umol, 114.76 uL, 4 eq), the mixture was stirred at 25° C. for 15 min, Then was added intermediate 1-5 (65.84 mg, 206.13 umol, 1 eq) and AcOH (74.27 mg, 1.24 mmol, 70.73 uL, 6 eq) stirred at 25° C. for 15 min, followed by addition of NaBH3CN (25.91 mg, 412.26 umol, 2 eq). The resulting mixture was stirred at 25° C. for 1.5 hrs. LCMS showed 17% 199-4 remained and 31% of desired mass was detected. The mixture was filtered and concentrated under reduced pressure to give a residue. The residue was purified by prep-HPLC (column: Phenomenex luna C18 150*25 mm*10 um; mobile phase: [water (FA)-ACN]; B %: 22%-52%, 10 min) and the eluent was lyophilized to give product. Compound 120 (11 mg, 12.83 umol, 6.22% yield, 99% purity) was obtained as a yellow solid and confirmed by LCMS, HNMR.

### Mass Found

LCMS: Retention time: 1.853 min, (M+H)=849.2

LCMS: Retention time: 0.488 min, (M+H)=849.5

### NMR Data

¹H NMR (400 MHz, DMSO-d6) δ=8.28 (s, 1H), 8.15-8.07 (m, 2H), 7.85 (s, 1H), 7.80 (d, J=4.8 Hz, 1H), 7.67 (s, 1H), 7.62-7.57 (m, 2H), 7.52-7.41 (m, 4H), 7.18-7.11 (m, 2H), 7.08-7.05 (m, 1H), 6.90 (s, 1H), 4.54-4.45 (m, 2H), 4.45-4.37 (m, 2H), 4.00 (s, 2H), 3.88 (s, 2H), 3.04 (d, J=4.4 Hz, 2H), 2.69 (d, J=9.2 Hz, 2H), 2.32-2.27 (m, 5H), 1.75 (s, 2H), 1.49 (s, 4H), 1.35-1.30 (m, 3H)

### Example 200. Synthesis of [[5-(5-(((9-ethyl-7-(4-methylthiophen-2-yl)-9H-carbazol-3-yl)methyl)amino)pentyl)-N-(6-(thiophene-2-sulfonamido)benzo[d]thiazol-2-yl)-5,6,7,8-tetrahydro-4H-pyrazolo[1,5-a][1,4]diazepine-2-carboxamide]] (Compound 121)

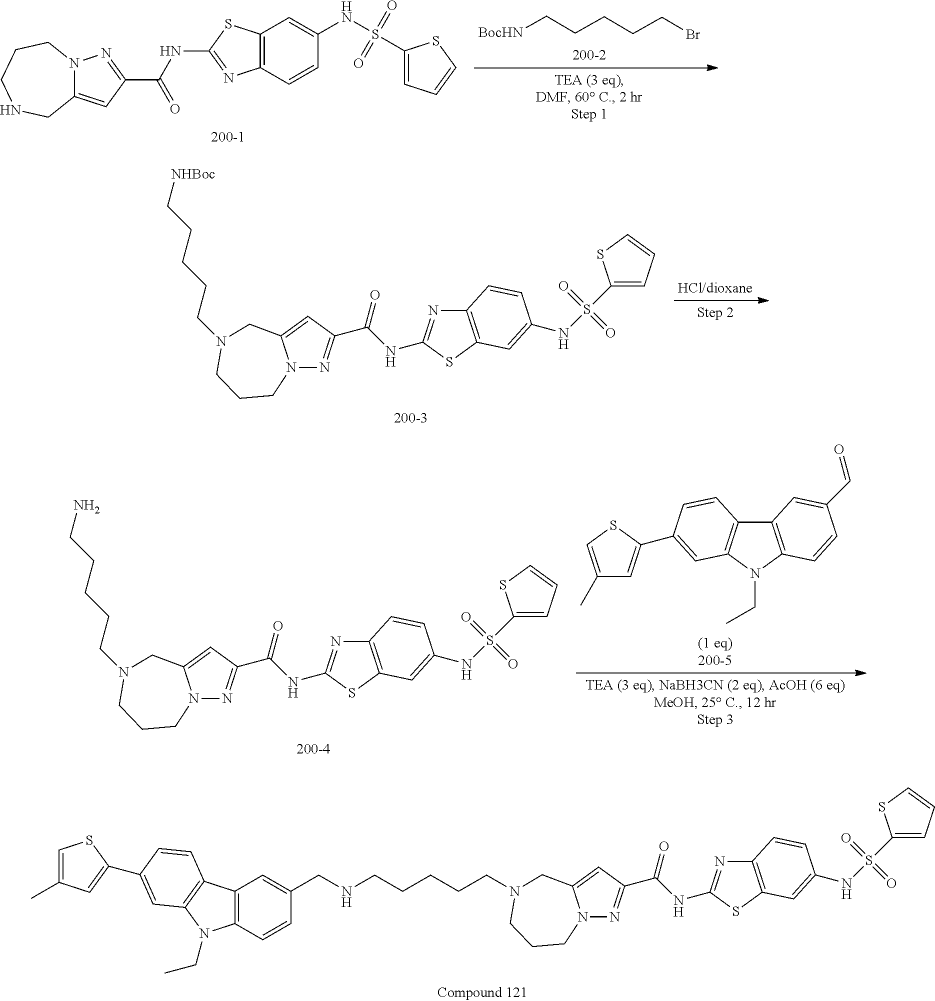

### Step 1: Synthesis of Intermediate 200-3

Detailed Synthetic Procedure: To a solution of intermediate 200-1 (0.3 g, 632.14 umol, 1 eq) and tert-butyl N-(5-bromopentyl)carbamate (504.78 mg, 1.90 mmol, 3 eq) in DMF (3 mL) was added TEA (191.90 mg, 1.90 mmol, 263.96 uL, 3 eq) at 25° C., then the mixture was stirred at 60° C. for 12 hrs. LCMS showed desired mass was detected. The mixture was quenched with H2O (5 mL) and extracted with EA 15 mL (5 mL*3). The organic layers were dried over Na2SO4, filtered and concentrated under reduced pressure to give a residue. The residue was purified by column chromatography (SiO2, PE/EA=100/0 to 0/100, EA:MeOH=10:1 Rt=0.4) and concentrated to give intermediate 200-3 (0.4 g, 606.21 umol, 95.90% yield) as yellow and confirmed by LCMS.

### Mass Found

LCMS: Retention time: 0.477 min, (M+H)=660.1

LCMS: Retention time: 0.475 min, (M+H)=660.1

### Step 2: Synthesis of Intermediate 200-4

Detailed Synthetic Procedure: To a solution of intermediate 200-3 (0.1 g, 151.55 umol, 1 eq) in dioxane (1 mL) was added HCl/dioxane (4 M, 2.00 mL, 52.79 eq), the mixture was stirred at 25° C. for 0.5 hr. LCMS showed a major peak with desired mass was detected. The mixture was filtered and concentrated to give yellow solid. The residue was taken to the next step without purification to give intermediate 200-4 (0.09 g, 150.96 umol, 99.61% yield, HCl) as a yellow solid.

### Mass Found

LCMS: Retention time: 0.296 min, (M+H)=560.1

### Step 3: Synthesis of Compound 121

Detailed Synthetic Procedure: To a solution of intermediate 200-4 (0.05 g, 83.87 umol, 1 eq, HCl) in MeOH (0.5 mL) was added TEA (25.46 mg, 251.60 umol, 35.02 uL, 3 eq), the mixture was stirred at 25° C. for 10 mins. Then AcOH (30.22 mg, 503.20 umol, 28.78 uL, 6 eq) and intermediate 200-5 (26.79 mg, 83.87 umol, 1 eq) was added to the mixture and stirred at 25° C. for 0.5 hr. The NaBH₃CN (15.81 mg, 251.60 umol, 3 eq) was added to the mixture and stirred at 25° C. for 12 hrs. LCMS showed 51.92% desired mass was detected. The mixture was filtered to give a residue. The residue was purified by preparative HPLC (column: Phenomenex luna C18 150*25 mm*10 um; mobile phase: [water (FA)-ACN]; B %:20%-50%, 10 min) and the eluent was lyophilized to give Compound 121 (12 mg, 13.75 umol, 16.40% yield, 98.92% purity) as yellow solid which was confirmed by LCMS and HNMR.

### Mass Found

LCMS: Retention time: 0.577 min, (M+H)=863.4

LCMS: Retention time: 0.681 min, (M+H)=863.1

### HNMR Data

¹H NMR (400 MHz, DMSO-d6) δ=8.33-8.25 (m, 1H), 8.13-8.07 (m, 2H), 7.84 (s, 1H), 7.80 (d, J=4.4 Hz, 1H), 7.67 (s, 1H), 7.61-7.56 (m, 2H), 7.50-7.42 (m, 4H), 7.18-7.10 (m, 2H), 7.06 (t, J=4.0 Hz, 1H), 6.89 (s, 1H), 4.50-4.39 (m, 4H), 4.00 (s, 2H), 3.87 (s, 2H), 3.04 (d, J=1.2 Hz, 2H), 2.28 (s, 7H), 1.75 (d, J=1.2 Hz, 2H), 1.51 (d, J=6.4 Hz, 2H), 1.45-1.39 (m, 2H), 1.34-1.27 (m, 5H), EC4072-379-P1A1.

### Example 201. Synthesis of 5-[7-[[9-ethyl-7-(4-methyl-2-thienyl)carbazol-3-yl]methylamino]heptyl]-N-[6-(2-thienylsulfonylamino)-1,3-benzothiazol-2-yl]-4,6,7,8-tetrahydropyrazolo[1,5-a][1,4]diazepine-2-carboxamide (Compound 122)

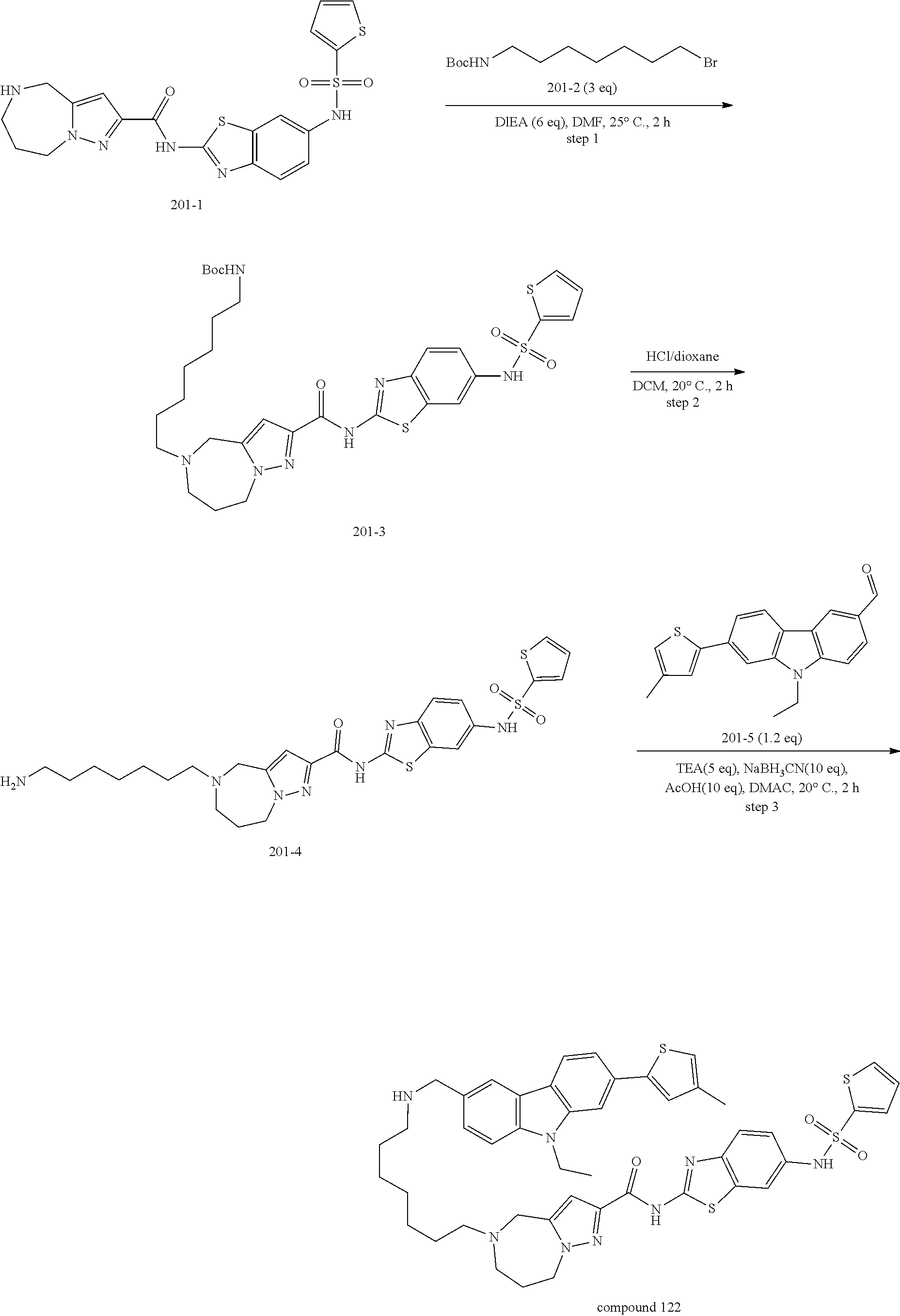 [SMILES: O=C(Nc1nc2ccc(NS(=O)(=O)c3cccs3)cc2s1)c1cc2n(n1)CCCNC2 ; CC(C)(C)OC(=O)NCCCCCCCBr ; CC(C)(C)OC(=O)NCCCCCCCN1CCCn2nc(C(=O)Nc3nc4ccc(NS(=O)(=O)c5cccs5)cc4s3)cc2C1 ; NCCCCCCCN1CCCn2nc(C(=O)Nc3nc4ccc(NS(=O)(=O)c5cccs5)cc4s3)cc2C1 ; CCn1c2ccc(C=O)cc2c2ccc(-c3cc(C)cs3)cc21 ; CCn1c2ccc(CNCCCCCCCN3CCCn4nc(C(=O)Nc5nc6ccc(NS(=O)(=O)c7cccs7)cc6s5)cc4C3)cc2c2ccc(-c3cc(C)cs3)cc21]

### Step 1: Synthesis of Intermediate 1-3

Detailed Synthetic Procedure: To a solution of intermediate 201-1 (150 mg, 293.52 umol, 1 eq, HCl) in DMF (1.5 mL) was added DIEA (227.61 mg, 1.76 mmol, 306.75 uL, 6 eq). The mixture was stirred at 25° C. for 10 min. Then intermediate 201-2 (259.08 mg, 880.56 umol, 3 eq) was added to the mixture and stirred at 25° C. for 2 hrs. LCMS showed 14% of desired molecular weight was detected. The mixture was diluted with MeOH (1 ml) and purified by reverse-phase directly (Combine flash (40 g of XB-C18, 20-35 μm, 100 Å) Mobile phase: A for H2O (0.1% FA v/v) and B for acetonitrile; Gradient: B 0%-80% in 15 min; Flow rate: 40 ml/min; Column temperature: R.T. Wavelength: 220 nm/254 nm) and the eluent was concentrated to remove MeCN and then lyophilized to afford intermediate 201-3 (55 mg, 79.95 umol, 27.24% yield, 100% purity) as a yellow solid which was confirmed by LCMS and ¹HNMR.

### Mass Found

LCMS: Rt=1.006 min, M+H=688.1

LCMS: Rt=0.789 min, M+H=688.0

### NMR Data

¹H NMR (400 MHz, DMSO-d6) δ=7.86 (d, J=4.4 Hz, 1H), 7.72 (d, J=2.0 Hz, 1H), 7.64 (d, J=8.8 Hz, 1H), 7.52-7.50 (m, 1H), 7.18-7.15 (m, 1H), 7.10-7.08 (m, 1H), 6.91 (s, 1H), 6.80-6.71 (m, 1H), 4.47-4.39 (m, 2H), 3.87 (s, 2H), 3.05 (d, J=4.0 Hz, 2H), 2.92-2.83 (m, 2H), 2.28-2.24 (m, 2H), 1.75 (s, 2H), 1.35 (s, 13H), 1.22 (s, 6H).

### Step 2: Synthesis of Intermediate 201-4

Detailed Synthetic Procedure: To a solution of intermediate 201-3 (55 mg, 79.95 umol, 1 eq) in DCM (1 mL) was added HCl/dioxane (4 M, 0.5 mL, 25.01 eq). The mixture was stirred at 25° C. for 2 hrs. LCMS showed one main peak with desired molecular weight was detected. The mixture was concentrated under reduced pressure to give the crude product. The crude product was used into the next step without further purification. Intermediate 201-4 (100 mg, 78.50 umol, 98.18% yield, 49% purity, HCl) was obtained as yellow gum.

### Step 3: Synthesis of Compound 122

Detailed Synthetic Procedure: To a solution of intermediate 201-4 (100 mg, 78.50 umol, 49% purity, 1 eq, HCl) and intermediate 201-5 (30.09 mg, 94.19 umol, 1.2 eq) in DMAC (1 mL) was added TEA (39.71 mg, 392.48 umol, 54.63 uL, 5 eq). The mixture was stirred at 25° C. for 10 min. Then AcOH (47.14 mg, 784.95 umol, 44.89 uL, 10 eq) was added to the mixture and the mixture stirred at 25° C. for 1 h. Then NaBH₃CN (73.99 mg, 1.18 mmol, 15 eq) was added to the mixture and the mixture stirred at 25° C. for 2 hrs. LCMS showed 19% of desired molecular weight was detected. The mixture diluted with MeOH (1 ml) and was purified by prep-HPLC directly (column: Phenomenex Luna C18 150*25 mm*10 um; mobile phase: [water (FA)-ACN]; B %: 20%-50%, 10 min) and the eluent was concentrated to remove MeCN and then lyophilized to afford Compound 122 (5.97 mg, 6.15 umol, 7.83% yield, 96.51% purity, FA) as an off-white gum which was confirmed by LCMS and ¹HNMR.

### Mass Found

LCMS: Rt=0.826 min, M+H=891.0

LCMS: Rt=0.886 min, M+H=891.4

### NMR Data

¹H NMR (400 MHz, DMSO-d6) δ=8.32 (s, 1H), 8.20-8.05 (m, 2H), 7.84 (s, 1H), 7.79-7.75 (m, 1H), 7.64 (d, J=2.0 Hz, 1H), 7.58-7.55 (m, 2H), 7.52-7.39 (m, 4H), 7.12 (s, 2H), 7.06-7.03 (m, 1H), 6.87 (s, 1H), 4.47 (d, J=6.8 Hz, 2H), 4.41 (d, J=2.4 Hz, 2H), 3.99 (s, 2H), 3.85 (s, 2H), 3.02 (d, J=0.8 Hz, 2H), 2.66 (s, 1H), 2.27 (s, 3H), 2.25-2.22 (m, 1H), 1.73 (s, 2H), 1.50 (s, 2H), 1.43-1.15 (m, 13H)

### Example 202. Synthesis of 5-[8-[[9-ethyl-7-(4-methyl-2-thienyl)carbazol-3-yl]methylamino]octyl]-N-[6-(2-thienylsulfonylamino)-1,3-benzothiazol-2-yl]-4,6,7,8-tetrahydropyrazolo[1,5-a][1,4]diazepine-2-carboxamide (Compound 123)

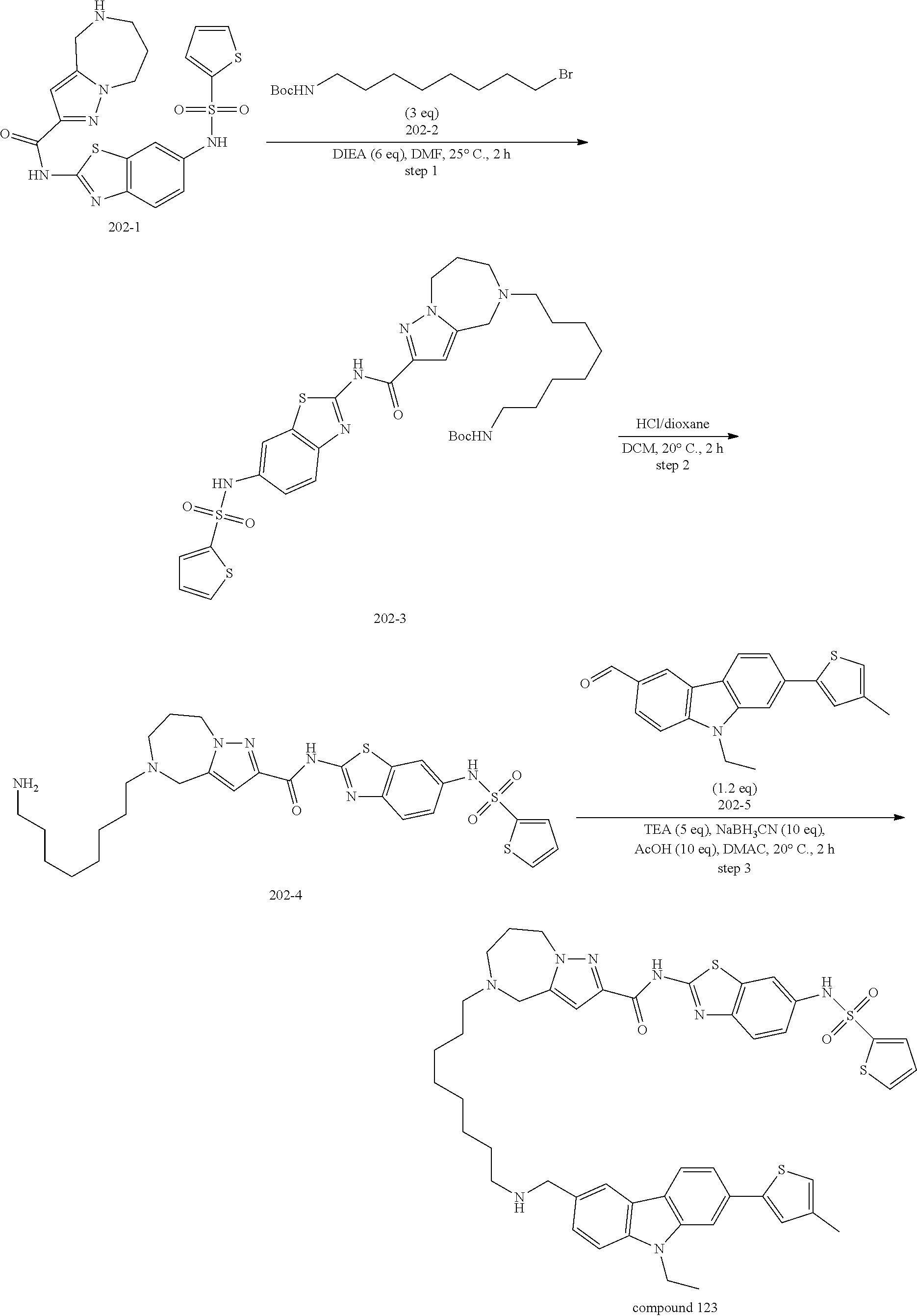 [SMILES: O=C(Nc1nc2ccc(NS(=O)(=O)c3cccs3)cc2s1)c1cc2n(n1)CCCNC2 ; CC(C)(C)OC(=O)NCCCCCCCCBr ; CC(C)(C)OC(=O)NCCCCCCCCN1CCCn2nc(C(=O)Nc3nc4ccc(NS(=O)(=O)c5cccs5)cc4s3)cc2C1 ; NCCCCCCCCN1CCCn2nc(C(=O)Nc3nc4ccc(NS(=O)(=O)c5cccs5)cc4s3)cc2C1 ; CCn1c2ccc(C=O)cc2c2ccc(-c3cc(C)cs3)cc21 ; CCn1c2ccc(CNCCCCCCCCN3CCCn4nc(C(=O)Nc5nc6ccc(NS(=O)(=O)c7cccs7)cc6s5)cc4C3)cc2c2ccc(-c3cc(C)cs3)cc21]

### Step 1: Synthesis of Intermediate 202-3

Detailed Synthetic Procedure: To a solution of intermediate 202-1 (150 mg, 293.52 umol, 1 eq, HCl) in DMF (1.5 mL) was added DIEA (227.61 mg, 1.76 mmol, 306.75 uL, 6 eq). The mixture was stirred at 20° C. for 10 min. Then intermediate 202-2 (271.44 mg, 880.56 umol, 3 eq) was added to the mixture and stirred at 20° C. for 2 hrs. LCMS showed 16% of desired molecular weight was detected. The mixture was diluted with MeOH (1 ml) and then purified by Prep-HPLC directly (Combine flash (40 g of XB-C18, 20-35 μm, 100 Å) Mobile phase: A for H2O (0.1% FA v/v) and B for acetonitrile; Gradient: B 0%-80% in 15 min; Flow rate: 40 ml/min; Column temperature: R.T. Wavelength: 220 nm/254 nm) and the eluent was concentrated to remove MeCN and then lyophilized to afford intermediate 202-3 (60 mg, 85.48 umol, 29.12% yield, 100% purity) as a yellow solid which was confirmed by LCMS and ¹HNMR Mass Found

LCMS: Rt=1.027 min, (M+H)=702.1

LCMS: Rt=0.805 min, (M+H)=702.0

### NMR Data

¹H NMR (400 MHz, DMSO-d6) δ=7.85 (s, 1H), 7.71 (s, 1H), 7.63 (d, J=8.8 Hz, 1H), 7.51 (d, J=2.8 Hz, 1H), 7.20-7.12 (m, 1H), 7.12-7.06 (m, 1H), 6.91 (s, 1H), 6.81-6.72 (m, 1H), 4.49-4.38 (m, 2H), 3.87 (s, 2H), 3.10-3.00 (m, 2H), 2.93-2.80 (m, 4H), 2.28-2.24 (m, 2H), 1.75 (d, J=4.0 Hz, 2H), 1.35 (s, 13H), 1.22 (s, 6H).

### Step 2: Synthesis of Intermediate 202-4

Detailed Synthetic Procedure: To a solution of intermediate 202-3 (60 mg, 85.48 umol, 1 eq) in DCM (1 mL) was added HCl/dioxane (4 M, 532.79 uL, 24.93 eq). The mixture was stirred at 25° C. for 2 hrs. LCMS showed one main peak with desired molecular weight was detected. The mixture was concentrated under reduced pressure to give the crude product. The crude product was used into the next step without further purification. Intermediate 202-4 (120 mg, 78.96 umol, 92.38% yield, 42% purity, HCl) was obtained as yellow gum.

### Mass Found

LCMS: Rt=0.802 min, (M+H)=602.1

### Step 3: Synthesis of Compound 123

Detailed Synthetic Procedure: To a solution of intermediate 202-4 (120 mg, 78.96 umol, 42% purity, 1 eq, HCl) and intermediate 202-5 (30.27 mg, 94.76 umol, 1.2 eq) in DMAC (1 mL) was added TEA (39.95 mg, 394.82 umol, 54.95 uL, 5 eq). The mixture was stirred at 25° C. for 10 min. Then AcOH (47.42 mg, 789.64 umol, 45.16 uL, 10 eq) was added to the mixture and the mixture stirred at 25° C. for 1 h. Then NaBH3CN (49.62 mg, 789.64 umol, 10 eq) was added to the mixture and the mixture stirred at 25° C. for 2 hrs. LCMS showed 30% of desired molecular weight was detected. The mixture was diluted with MeOH (1 ml) and then purified by prep-HPLC directly (column: Phenomenex luna C18 150*25 mm*10 um; mobile phase: [water (FA)-ACN]; B %: 20%-50%, 10 min) and the eluent was concentrated to remove MeCN and then lyophilized to afford Compound 123 (5.71 mg, 6.31 umol, 7.99% yield, 100% purity) as off-white gum which was confirmed by LCMS and ¹HNMR.

### Mass Found

LCMS: Rt=0.829 min, (M+H)=905.0

LCMS: Rt=0.806 min, (M+H)=905.5

### NMR Data

¹H NMR (400 MHz, DMSO-d6) δ=8.31 (s, 1H), 8.14 (s, 1H), 8.08 (d, J=8.0 Hz, 1H), 7.85 (s, 1H), 7.82-7.76 (m, 1H), 7.67 (s, 1H), 7.63-7.54 (m, 2H), 7.53-7.38 (m, 3H), 7.20-7.09 (m, 2H), 7.06 (s, 1H), 6.87 (s, 1H), 4.53-4.35 (m, 4H), 4.06 (s, 2H), 3.85 (s, 2H), 3.03 (s, 2H), 2.72 (d, J=1.2 Hz, 2H), 2.27 (s, 3H), 2.07 (s, 2H), 1.81-1.65 (m, 2H), 1.61-1.47 (m, 2H), 1.41-1.28 (m, 5H), 1.23 (s, 6H), 1.07-1.03 (m, 2H)

### Example 203. Synthesis of 5-(2-(2-(2-(((9-ethyl-7-(4-methylthiophen-2-yl)-9H-carbazol-3-yl)methyl)amino)ethoxy)ethoxy)ethyl)-N-(6-(thiophene-2-sulfonamido)benzo[d]thiazol-2-yl)-5,6,7,8-tetrahydro-4H-pyrazolo[1,5-a][1,4]diazepine-2-carboxamide (Compound 124)

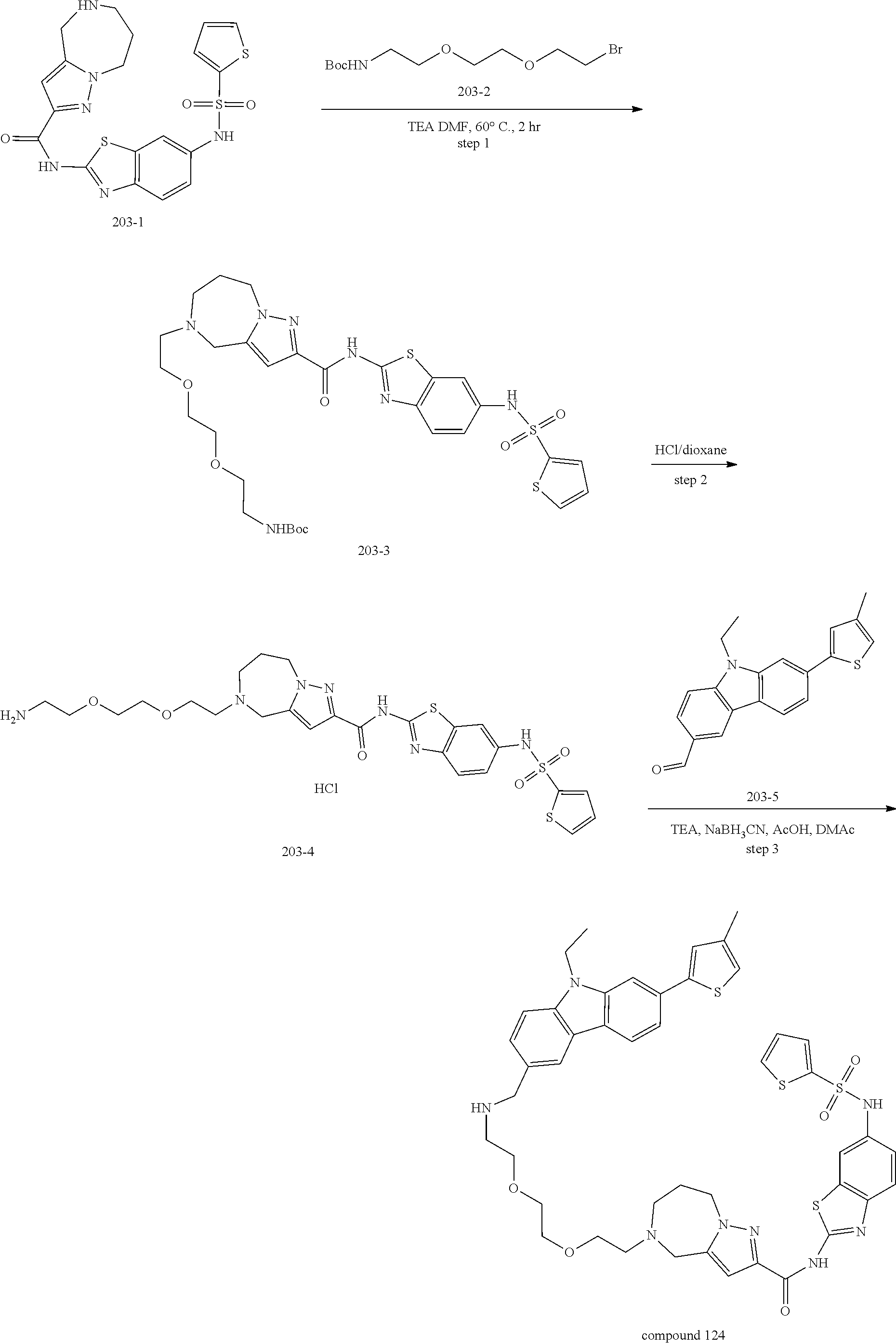

### Step 1: Synthesis of Intermediate 203-3

To a solution of Intermediate 203-1 (150 mg, 293.52 umol, 1 eq, HCl) and Intermediate 203-2 (274.91 mg, 880.56 umol, 3 eq) in DMF (1 mL) was added TEA (63.97 mg, 632.14 umol, 87.99 uL, 3 eq). The mixture was stirred at 60° C. for 2 hr. LCMS showed Reactant 1 was consumed and desired mass was detected. The reaction mixture was filtered to get the filtrate. The filtrate was purified by reverse phase column (FA) to give product Intermediate 203-3 (100 mg, 119.71 umol, 40.78% yield, 84.5% purity) as a white solid which was confirmed by LCMS.

### Mass Found

LCMS: Retention time: 0.761 min, (M+H)=706.0

LCMS: Retention time: 0.760 min, (M+H)=706.0

### Step 2: Synthesis of Intermediate 203-4

A solution of Intermediate 203-3 (100 mg, 141.67 umol, 1 eq) in HCl/dioxane (4 M, 1 mL) was stirred at 25° C. for 1 hr. TLC (PE:EA=1:1) showed 203-3 (Rf=0.5) was consumed and one new major spot (Rf=0) was detected. The mixture was filtered and concentrated under reduced pressure to give a residue. The crude product was used into the next step without further purification. Intermediate 203-4 (90 mg, 130.89 umol, 92.39% yield, 93.4% purity, HCl) as a white solid which was detected by LCMS.

### Mass Found

LCMS: Retention time: 0.626 min, (M+H)=606.0

### Step 3: Synthesis of Compound 124

Detailed Synthetic Procedure: To a solution of Intermediate 203-4 (50 mg, 77.86 umol, 1 eq, HCl) in DMAC (1 mL) was added TEA (39.39 mg, 389.28 umol, 54.18 uL, 5 eq), the mixture was stirred at 25° C. for 1 hr, then Intermediate 203-5 (22.38 mg, 70.07 umol, 0.9 eq) and AcOH (46.75 mg, 778.56 umol, 44.53 uL, 10 eq) was added and the mixture was stirred at 25° C. for 15 min, followed by addition of NaBH3CN (48.73 mg, 778.56 umol, 10 eq). The resulting mixture was stirred at 25° C. for 14 hr. LCMS showed Reactant 1 was consumed and desired mass (Rt=0.936) was detected. The mixture was filtered and concentrated under reduced pressure to give a residue. The residue was purified by prep-HPLC (column: 3_Phenomenex Luna C18 75*30 mm*3 um; mobile phase: [water(HCl)-ACN];B %: 24%-44%, 8 min) and the eluent was lyophilized to give product. Compound 124 (22 mg, 22.92 umol, 29.43% yield, 98.5% purity,) as a yellow solid confirmed by LCMS, HNMR.

### Mass Found

LCMS: Retention time: 0.817 min, (M+H)=909.0

LCMS: Retention time: 0.760 min, (M+H)=909.2

### NMR Data

¹H NMR (400 MHz, DMSO-d6) δ=12.58-12.30 (m, 1H), 11.56-11.26 (m, 1H), 10.50 (s, 1H), 9.28 (br s, 2H), 8.31 (s, 1H), 8.07 (d, J=8.0 Hz, 1H), 7.92-7.84 (m, 2H), 7.76 (d, J=1.6 Hz, 1H), 7.70-7.62 (m, 3H), 7.56-7.42 (m, 3H), 7.26-7.08 (m, 4H), 4.71 (br s, 2H), 4.59-4.47 (m, 4H), 4.33 (br s, 2H), 3.87-3.77 (m, 4H), 3.65 (s, 6H), 3.14 (br d, J=4.4 Hz, 4H), 2.27 (s, 3H), 2.15 (br s, 2H), 1.33-1.31 (m, 3H).

### Example 204. Synthesis of 5-(2-(2-(((9-ethyl-7-(4-methylthiophen-2-yl)-9H-carbazol-3-yl)methyl)amino)ethoxy)ethyl)-N-(6-(thiophene-2-sulfonamido)benzo[d]thiazol-2-yl)-5,6,7,8-tetrahydro-4H-pyrazolo[1,5-a][1,4]diazepine-2-carboxamide (Compound 125)

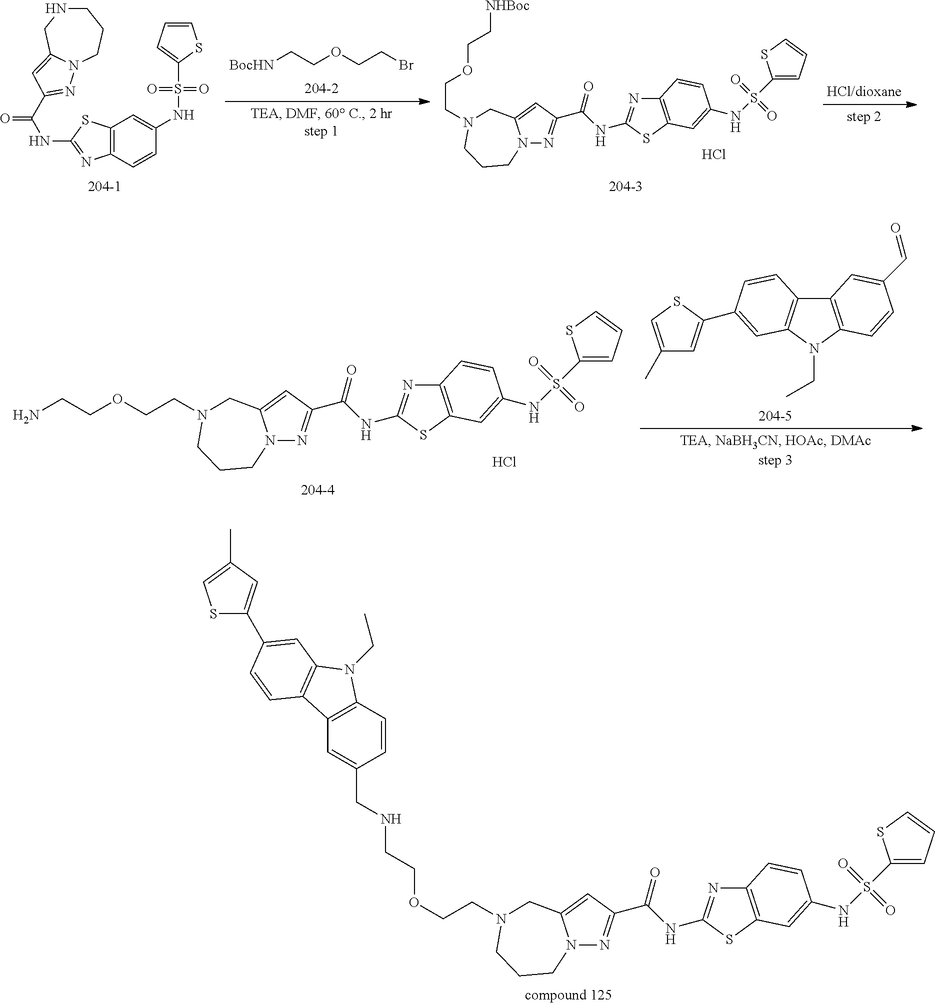

### Step 1: Synthesis of Intermediate 1-3

To a solution of Intermediate 204-1 (150 mg, 293.52 umol, 1 eq, HCl) and Intermediate 204-2 (236.12 mg, 880.56 umol, 3 eq) in DMF (1 mL) was added TEA (63.97 mg, 632.14 umol, 87.99 uL, 3 eq). The mixture was stirred at 60° C. for 2 hr. LCMS showed Reactant 1 was consumed and desired mass was detected. The reaction mixture was filtered to get the filtrate. The filtrate was purified by reverse phase (FA) to get product Intermediate 204-3 (100 mg, 151.10 umol, 42.98% yield, 83.5% purity) as a white solid which was confirmed by LCMS.

### Mass Found

LCMS: Retention time: 0.755 min, (M+H)=662.0

LCMS: Retention time: 0.755 min, (M+H)=662.0

### Step 2: Synthesis of Intermediate 204-4

Detailed Synthetic Procedure: A solution of Intermediate 204-3 (150 mg, 151.10 umol, 1 eq) in HCl/dioxane (4 M, 1 mL) was stirred at 25° C. for 1 hr. TLC (PE:EA=1:1) showed Reactant 1 (Rf=0.5) was consumed and one new major spot (Rf=0) was detected. The mixture was filtered and concentrated under reduced pressure to give a residue. The crude product was used into the next step without further purification. Intermediate 204-4 (90 mg, 137.22 umol, 90.81% yield, 91.2% purity, HCl) as a white solid which was confirmed by LCMS.

### Mass Found

LCMS: Retention time: 0.622 min, (M+H)=562.0

### Step 3: Synthesis of Compound 1

Detailed Synthetic Procedure: To a solution of Intermediate 204-4 (50 mg, 83.59 umol, 1 eq, HCl) in DMAC (1 mL) was added TEA (42.29 mg, 417.59 umol, 54.18 uL, 5 eq), the mixture was stirred at 25° C. for 1 hr, then Intermediate 204-5 (24.03 mg, 75.23 umol, 0.9 eq) and AcOH (50.20 mg, 835.90 umol, 47.81 uL, 10 eq) was added and the mixture was stirred at 25° C. for 15 min, followed by addition of NaBH₃CN (52.53 mg, 835.90 umol, 10 eq). The resulting mixture was stirred at 25° C. for 14 hr. LCMS showed Reactant 1 was consumed and desired mass was detected. The mixture was filtered and concentrated under reduced pressure to give a residue. The residue was purified by prep-HPLC (column: Phenomenex Luna C18 75*30 mm*3 um; mobile phase: [water (HCl)-ACN];B %: 24%-44%, 8 min) and the eluent was lyophilized to give product. Compound 125 (16 mg, 17.75 umol, 21.23% yield, 100% purity, HCl) as a yellow solid which was confirmed by LCMS, HNMR.

### Mass Found

LCMS: Retention time: 0.820 min, (M+H)=864.8

LCMS: Retention time: 0.755 min, (M+H)=865.2

### NMR Data

¹H NMR (400 MHz, DMSO-d6) δ=12.59-12.16 (m, 1H), 11.28 (br d, J=1.6 Hz, 1H), 10.51 (s, 1H), 9.57 (br d, J=0.8 Hz, 2H), 8.39 (s, 1H), 8.08 (d, J=8.0 Hz, 1H), 7.93-7.84 (m, 2H), 7.80-7.71 (m, 2H), 7.70-7.62 (m, 2H), 7.57-7.46 (m, 3H), 7.29 (s, 1H), 7.21-7.19 (m, 1H), 7.16-7.07 (m, 2H), 4.80 (br s, 2H), 4.72-4.58 (m, 2H), 4.56-4.48 (m, 2H), 4.37 (br s, 2H), 3.84-3.66 (m, 6H), 3.17 (br d, J=3.2 Hz, 4H), 2.28 (s, 3H), 2.20 (br s, 2H), 1.34-1.32 (m, 3H)

### Example 205. Synthesis of [[2-[(2E)-2-[5-oxo-3-phenyl-1-(4-phenylthiazol-2-yl)pyrazol-4-ylidene]hydrazino]-N-[3-[2-[[6-(2-thienylsulfonylamino)-1,3-benzothiazol-2-yl]carbamoyl]-4,6,7,8-tetrahydropyrazolo[1,5-a][1,4]diazepin-5-yl]propyl]thiazole-5-carboxamide]](Compound 126)

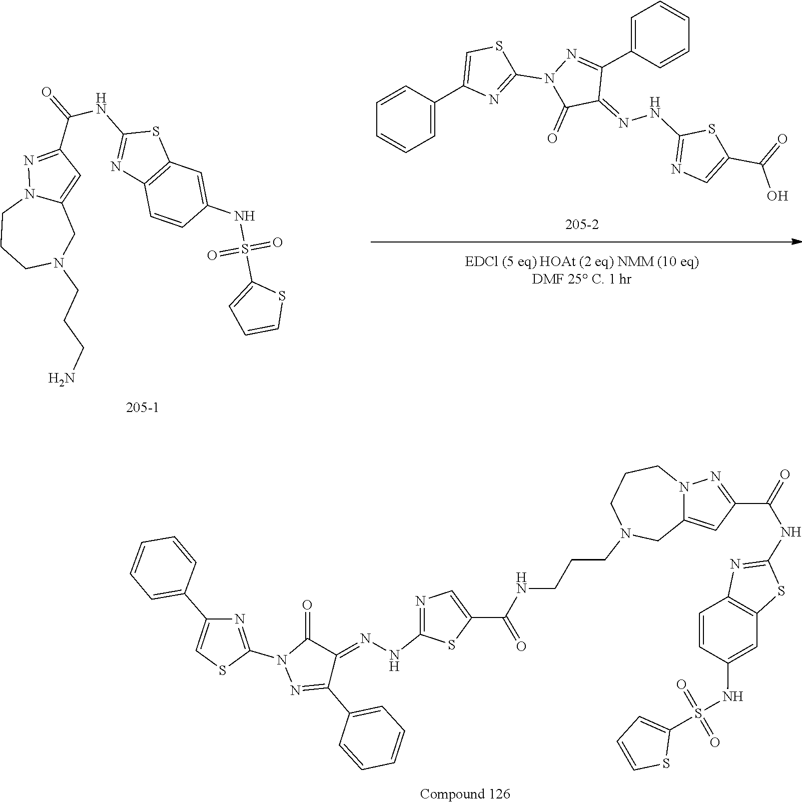

### Step 1: Synthesis of Compound 126

To a solution of intermediate 205-1 (20 mg, 35.20 umol, 1 eq, HCl) in DMF (0.5 mL) was added HOAt (9.58 mg, 70.41 umol, 9.85 uL, 2 eq), EDCI (33.74 mg, 176.01 umol, 5 eq) and NN (35.61 mg, 352.03 umol, 38.70 uL, 10 eq) which was added intermediate 205-2 (20.05 mg, 42.24 umol, 1.2 eq). The mixture was stirred at 25° C. for 1 hr. LCMS showed desired mass was detected. The mixture was concentrated under reduced pressure to give a residue. The residue was purified by prep-HPLC (column: Welch Ultimate C18 150*25 mm*5 um;mobile phase: [water(TFA)-ACN];B %: 34%-64%, 10 min), which was lyophilizated to give Compound 126 (8.5 mg, 8.60 umol, 24.43% yield, 100% purity) as orange solid which was confirmed by LCMS, FNMR, and HNMR.

### Mass Found

LCMS: Retention time: 0.475 min, (M+H)=988.1

LCMS: Retention time: 0.469 min, (M+H)=988.2

### NMR Data

¹H NMR (400 MHz, DMSO-d6) δ=12.58-12.36 (m, 1H), 10.46 (s, 1H), 10.29-9.98 (m, 1H), 8.53 (t, J=5.2 Hz, 1H), 8.19-8.10 (m, 3H), 8.00 (d, J=7.6 Hz, 2H), 7.87 (d, J=4.8 Hz, 1H), 7.76 (d, J=10.0 Hz, 2H), 7.66 (d, J=8.8 Hz, 1H), 7.54-7.41 (m, 6H), 7.38-7.31 (m, 1H), 7.24 (s, 1H), 7.19 (d, J=8.8 Hz, 1H), 7.09 (t, J=4.4 Hz, 1H), 4.95-4.76 (m, 2H), 4.61 (s, 2H), 3.45-3.11 (m, 6H), 2.29-2.08 (m, 2H), 1.89 (s, 2H).

### Example 206. Synthesis of (E)-2-(2-(5-oxo-3-phenyl-1-(4-phenylthiazol-2-yl)-1H-pyrazol-4(5H)-ylidene)hydrazinyl)-N-(6-(2-((6-(thiophene-2-sulfonamido)benzo[d]thiazol-2-yl)carbamoyl)-7,8-dihydro-4H-pyrazolo[1,5-a][1,4]diazepin-5(6H)-yl)hexyl)thiazole-5-carboxamide (Compound 127)

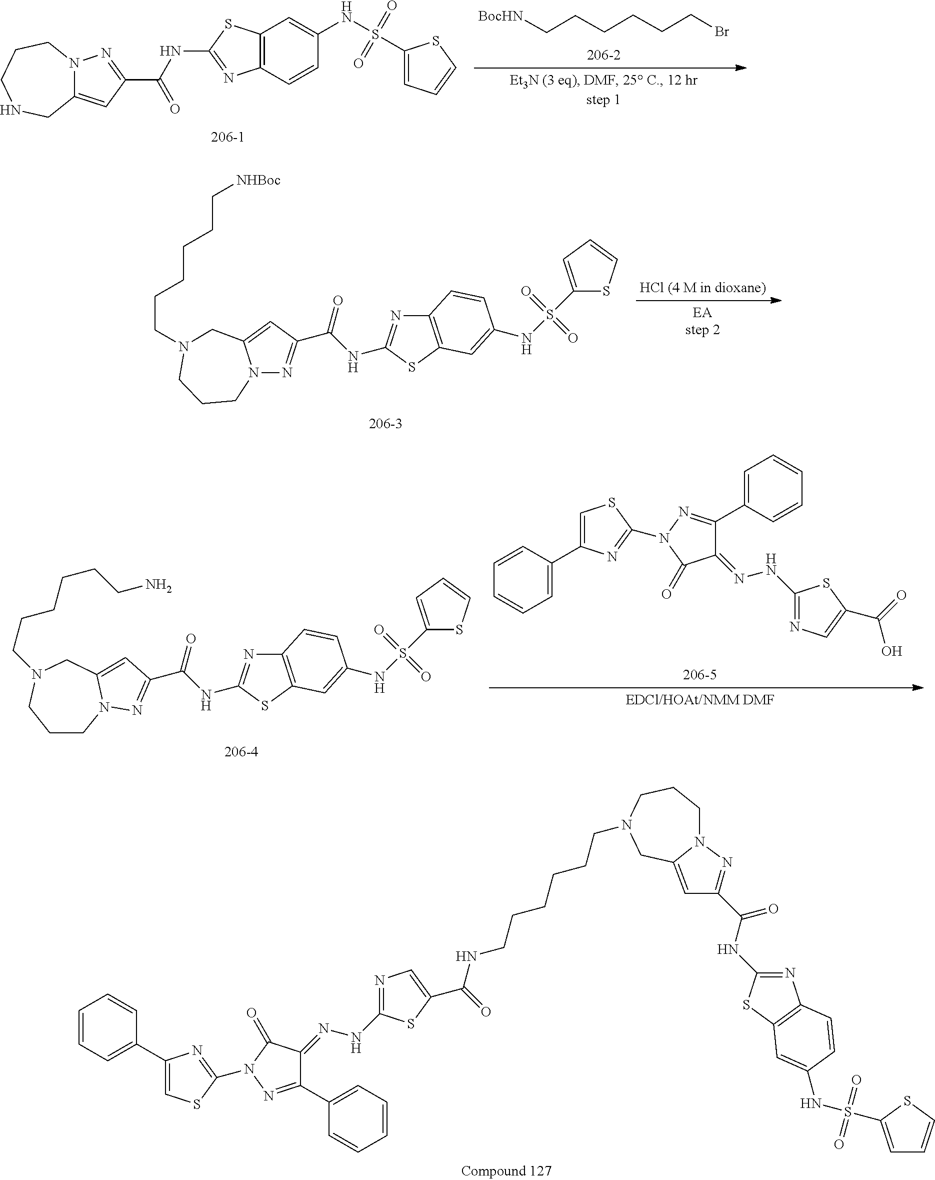

### Step 1: Synthesis of Intermediate 206-3

To a solution of intermediate 206-1 (100 mg, 210.71 umol, 1 eq.) in DMF (1.5 mL) was added Et₃N (63.97 mg, 632.14 umol, 87.99 uL, 3 eq.) and intermediate 206-2 (177.13 mg, 632.14 umol, 3 eq.). The mixture was stirred at 60° C. for 5 h. LC-MS showed a main peak with desired mass was detected. The mixture was quenched by H₂O (5 ml) and extracted with EA (3×8 mL). The organic phase was concentrated under reduced pressure to get intermediate 206-3 as a white solid (200 mg, 198.85 umol, 94.37% yield, 67% purity), which was used directly in the next step.

### Mass:

Retention time: 0.401 min, (M+H)=674.6

### Step 2: Synthesis of Intermediate 206-4

Detailed Synthetic Procedure: To a solution of intermediate 206-3 (100 mg, 99.43 umol, 67% purity, 1 eq.) in EA (0.4 mL) was added HCl/dioxane (4 M, 0.4 mL, 16.09 eq.). The mixture was stirred at 25° C. for 0.5 h. LC-MS showed a major peak with mass was detected. The reaction mixture was concentrated under reduced pressure to get intermediate 206-4 (90 mg, crude, HCl salt) as a white solid, which was used without purification.

### Mass:

Retention time: 0.308 min, (M+H)=574.0

### Step 3: Synthesis of Compound 1

Detailed Synthetic Procedure: To a solution of intermediate 1-5 (60 mg, 104.57 umol, 1.5 eq.) in DMF (0.6 mL) was added NMM (35.26 mg, 348.58 umol, 38.32 uL, 5 eq), intermediate 1-4 (33.08 mg, 69.72 umol, 1 eq.), HOAt (9.49 mg, 69.72 umol, 9.75 uL, 1 eq) and EDCI (20.05 mg, 104.57 umol, 1.5 eq). The mixture was stirred at 25° C. for 1 h. LC-MS (EC4074-236-P1A2) showed desired mass was detected. The mixture was diluted with H₂O (3 mL) and DMSO (6 mL) and the red precipitate was collected by filtration. The obtained red solid was purified by Prep-HPLC (column: Phenomenex luna C18 150*25 mm*10 um; mobile phase: [water(TFA)-ACN];B %: 40%-70%, 10 min). The eluent was concentrated and lyophilized to get Compound 1 (7 mg, 6.45 umol, 9.26% yield, 95% purity) as a red solid, which was confirmed by ¹H NMR (EC4074-236-P1A2) and LCMS (EC4074-236-P1J1).

### Mass:

Retention time: 0.441 min, (M+H)=1030.2, 5-95AB_R_220&254.1 cm, (EC4074-236-P1A2)

Retention time: 0.502 min, (M+H)=1030.1, 5-95AB_R_220&254.1 cm, (EC4074-236-P1J1)

### NMR Data:

¹H NMR (400 MHz, DMSO-d₆+D₂O) δ=8.17-8.07 (m, 3H), 7.96 (d, J=7.6 Hz, 2H), 7.78 (d, J=3.6 Hz, 1H), 7.70-7.62 (m, 3H), 7.52-7.41 (m, 6H), 7.35-7.30 (m, 1H), 7.20-7.14 (m, 2H), 7.07 (t, J=3.6 Hz, 1H), 4.66-4.59 (m, 2H), 4.55 (d, J=4.4 Hz, 2H), 3.59-3.54 (m, 2H), 3.23-3.18 (m, 2H), 3.03-2.97 (m, 2H), 1.70-1.62 (m, 2H), 1.53-1.46 (m, 2H), 1.33-1.26 (m, 4H), 1.21-1.17 (m, 2H) (EC4074-236-P1A2)

### Example 207. Synthesis of [[(E)-2-(2-(5-oxo-3-phenyl-1-(4-phenylthiazol-2-yl)-1H-pyrazol-4(5H)-ylidene)hydrazinyl)-N-(7-(2-((6-(thiophene-2-sulfonamido)benzo[d]thiazol-2-yl)carbamoyl)-7,8-dihydro-4H-pyrazolo[1,5-a][1,4]diazepin-5(6H)-yl)heptyl)thiazole-5-carboxamide]] (Compound 128)

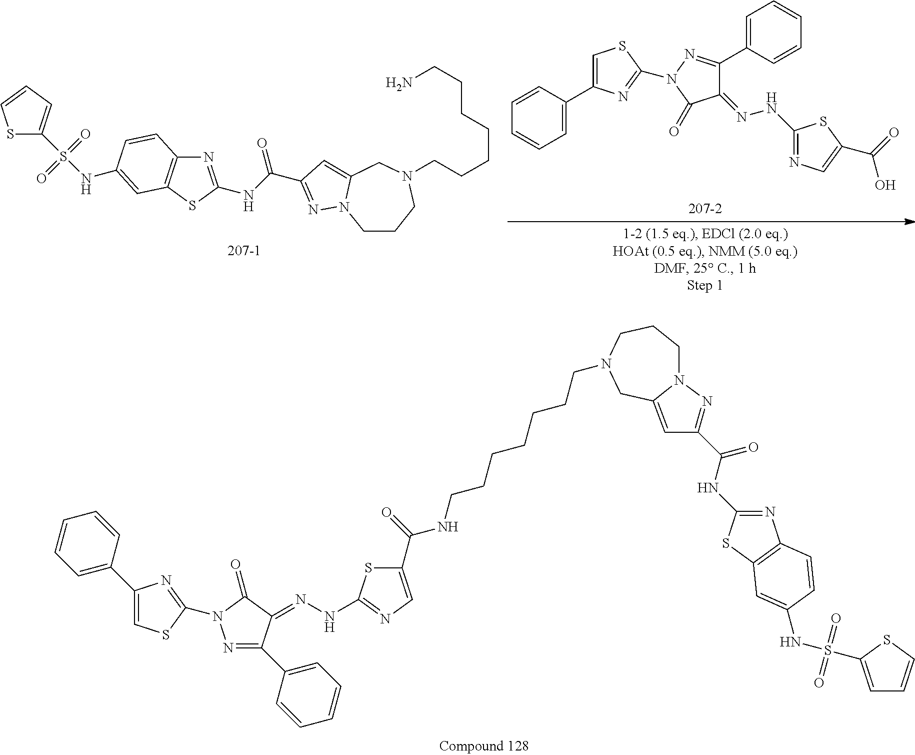

### Step 1: Synthesis of Compound 128

To a solution of Intermediate 207-1 (0.02 g, 32.04 umol, 1 eq., HCl salt) and Intermediate 207-2 (22.80 mg, 48.06 umol, 1.5 eq.) in DMF (0.2 mL) was added EDCI (12.28 mg, 64.08 umol, 2 eq.), HOAt (2.18 mg, 16.02 umol, 2.24 uL, 0.5 eq.) and NMM (16.20 mg, 160.19 umol, 17.61 uL, 5 eq.). Then the mixture was stirred at 25° C. for 1 h. LCMS showed 65% desired mass was detected. The mixture was filtered to give a residue. The residue was purified by preparative HPLC (column: Phenomenex Luna C18 150*30 mm*5 um;mobile phase: [water(TFA)-ACN];B %: 45%-75%, 10 min) and lyophilized to give Compound 128 (10 mg, 9.32 umol, 29.08% yield, 97.29% purity) as orange solid, which was confirmed by LCMS and HNMR.

### Mass Found

LCMS: Retention time: 0.530 min, (M+H)=1044.1

LCMS: Retention time: 0.510 min, (M+H)=1044.1

### HNMR Data

1H NMR (400 MHz, DMSO-d₆) δ=10.49-10.44 (m, 1H), 8.50-8.38 (m, 1H), 8.23-8.10 (m, 3H), 8.03-7.98 (m, 2H), 7.88 (d, J=3.2 Hz, 1H), 7.76 (s, 2H), 7.67 (d, J=8.8 Hz, 1H), 7.57-7.51 (m, 3H), 7.47 (t, J=7.6 Hz, 3H), 7.38-7.34 (m, 1H), 7.25 (s, 1H), 7.20 (d, J=8.0 Hz, 1H), 7.10 (t, J=4.0 Hz, 1H), 4.83-4.62 (m, 2H), 4.59 (s, 2H), 3.24 (d, J=5.6 Hz, 2H), 3.10-3.00 (m, 2H), 2.27-2.23 (m, 2H), 1.73-1.65 (m, 2H), 1.56-1.50 (m, 2H), 1.43-1.22 (m, 8H).

### Example 208. Synthesis of [2-[(2E)-2-[5-oxo-3-phenyl-1-(4-phenylthiazol-2-yl)pyrazol-4-ylidene]hydrazino]-N-[2-[2-[2-[[6-(2-thienylsulfonylamino)-1,3-benzothiazol-2-yl]carbamoyl]-4,6,7,8-tetrahydropyrazolo[1,5-a][1,4]diazepin-5-yl]ethoxy]ethyl]thiazole-5-carboxamide] (Compound 129)

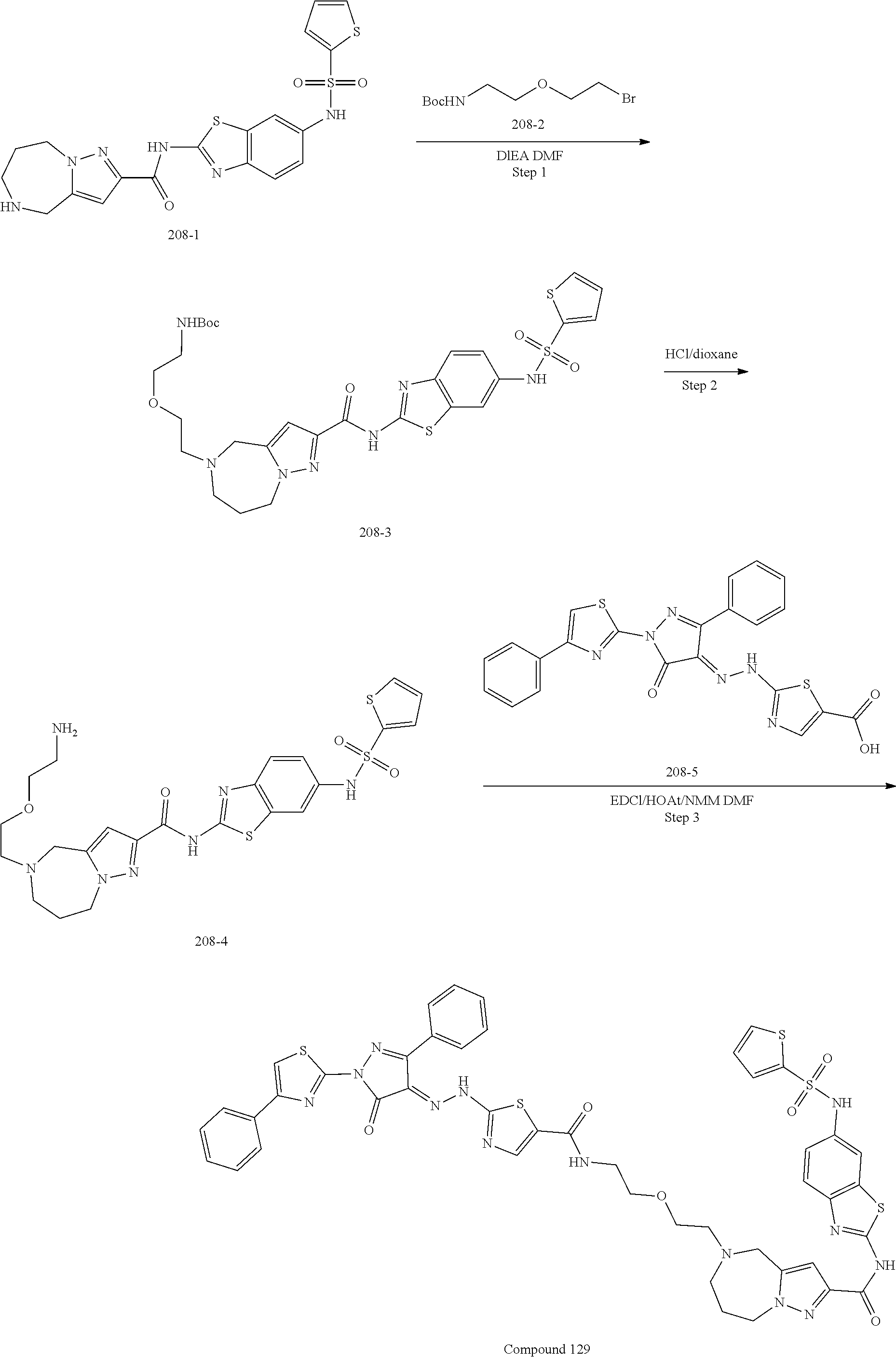 [SMILES: O=C(Nc1nc2ccc(NS(=O)(=O)c3cccs3)cc2s1)c1cc2n(n1)CCCNC2 ; CC(C)(C)OC(=O)NCCOCCBr ; CC(C)(C)OC(=O)NCCOCCN1CCCn2nc(C(=O)Nc3nc4ccc(NS(=O)(=O)c5cccs5)cc4s3)cc2C1 ; NCCOCCN1CCCn2nc(C(=O)Nc3nc4ccc(NS(=O)(=O)c5cccs5)cc4s3)cc2C1 ; O=C(O)c1cnc(N/N=C2/C(=O)N(c3nc(-c4ccccc4)cs3)N=C2c2ccccc2)s1 ; O=C(Nc1nc2ccc(NS(=O)(=O)c3cccs3)cc2s1)c1cc2n(n1)CCCN(CCOCCNC(=O)c1cnc(N/N=C3/C(=O)N(c4nc(-c5ccccc5)cs4)N=C3c3ccccc3)s1)C2]

### Step 1: Synthesis of Intermediate 208-3

To a solution of intermediate 208-1 (100 mg, 210.71 umol, 1 eq) and intermediate 208-2 (169.51 mg, 632.14 umol, 3 eq) in DMF (1 mL) was added TEA (63.97 mg, 632.14 umol, 87.99 uL, 3 eq). The mixture was stirred at 60° C. for 2 hrs. LCMS showed reactant was consumed completely and 64% of desired mass was detected. The reaction mixture was diluted with water (10 mL) and extracted with DCM (10 mL*3). The combined organic layer was dried over Na₂SO₄, filtered and concentrated in vacuo to give a residue. The residue was purified by column chromatography (SiO2, DCM:MeOH=20:1 to DCM:MeOH=10:1), then the organic liquid was concentrated in vacuo to give intermediate 208-3 (200 mg, 202.47 umol, 96.09% yield, 67% purity) was obtained as light yellow oil, which was confirmed by LCMS.

### Mass Found

Retention time=0.457 min, (M+H)=662.1

Retention time=0.391 min, (M+H)=662.3

### Step 2: Synthesis of Intermediate 208-4

Detailed Synthetic Procedure: To a solution of intermediate 208-3 (150 mg, 226.65 umol, 1 eq) in DCM (1 mL) was added HCl/dioxane (4 M, 56.66 uL, 1 eq). The mixture was stirred at 25° C. for 0.5 hr. LCMS showed reactant was consumed completely and 58% of desired mass was detected. The reaction mixture was concentrated in vacuo to give intermediate 208-4 (200 mg, 193.93 umol, 85.56% yield, 58% purity, HCl) was obtained as a white solid.

### Mass:

Retention time=0.320 min, (M+H)=562.1

### Step 3: Synthesis of Compound 129

Detailed Synthetic Procedure: To a solution of intermediate 208-4 (40 mg, 71.21 umol, 1 eq) and intermediate 208-5 (43.93 mg, 92.58 umol, 1.3 eq) in DMF (1 mL) was added EDCI (40.95 mg, 213.64 umol, 3 eq), NN (36.01 mg, 356.06 umol, 39.15 uL, 5 eq) and HOAt (9.69 mg, 71.21 umol, 9.96 uL, 1 eq). The mixture was stirred at 25° C. for 1 hr. LCMS showed reactant was consumed completely and 43% of desired mass was detected. The mixture was purified by prep-HPLC (column: Welch Ultimate C18 150*25 mm*5 um;mobile phase: [water(TFA)-ACN];B %: 35%-65%, 10 min) to give a residue. Then residue was concentrated in vacuo and lyophilized to give Compound 129 (8.63 mg, 8.48 umol, 11.90% yield, 100% purity) was obtained as a orange solid, which was confirmed by LCMS, HNMR, FNMR and 2D NMR.

### Mass:

Retention time=0.475 min, (M+H)=1018.1

Retention time=0.486 min, (M+H)=1018.0

### NMR Data:

¹H NMR (400 MHz, DMSO+D2O) δ=8.17-8.11 (m, 3H), 7.98 (d, J=7.2 Hz, 2H), 7.84-7.83 (m, 1H), 7.75 (s, 1H), 7.71 (d, J=2.0 Hz, 1H), 7.64 (d, J=8.8 Hz, 1H), 7.52-7.51 (m, 2H), 7.50-7.47 (m, 2H), 7.46-7.43 (m, 2H), 7.35 (d, J=7.6 Hz, 1H), 7.22 (s, 1H), 7.18-7.14 (m, 1H), 7.09-7.07 (m, 1H), 4.77-4.64 (m, 2H), 4.57 (br s, 2H), 3.80-3.74 (m, 2H), 3.63 (br d, J=5.6 Hz, 4H), 3.49 (br s, 2H), 3.28-3.24 (m, 2H), 2.18-2.10 (m, 2H).

### Example 209. Synthesis of [[N-[3-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-3-oxo-propyl]-4-[2-[(4E)-5-oxo-3-phenyl-4-(thiazol-2-ylhydrazono)pyrazol-1-yl]thiazol-4-yl]benzamide]] (Compound 130)

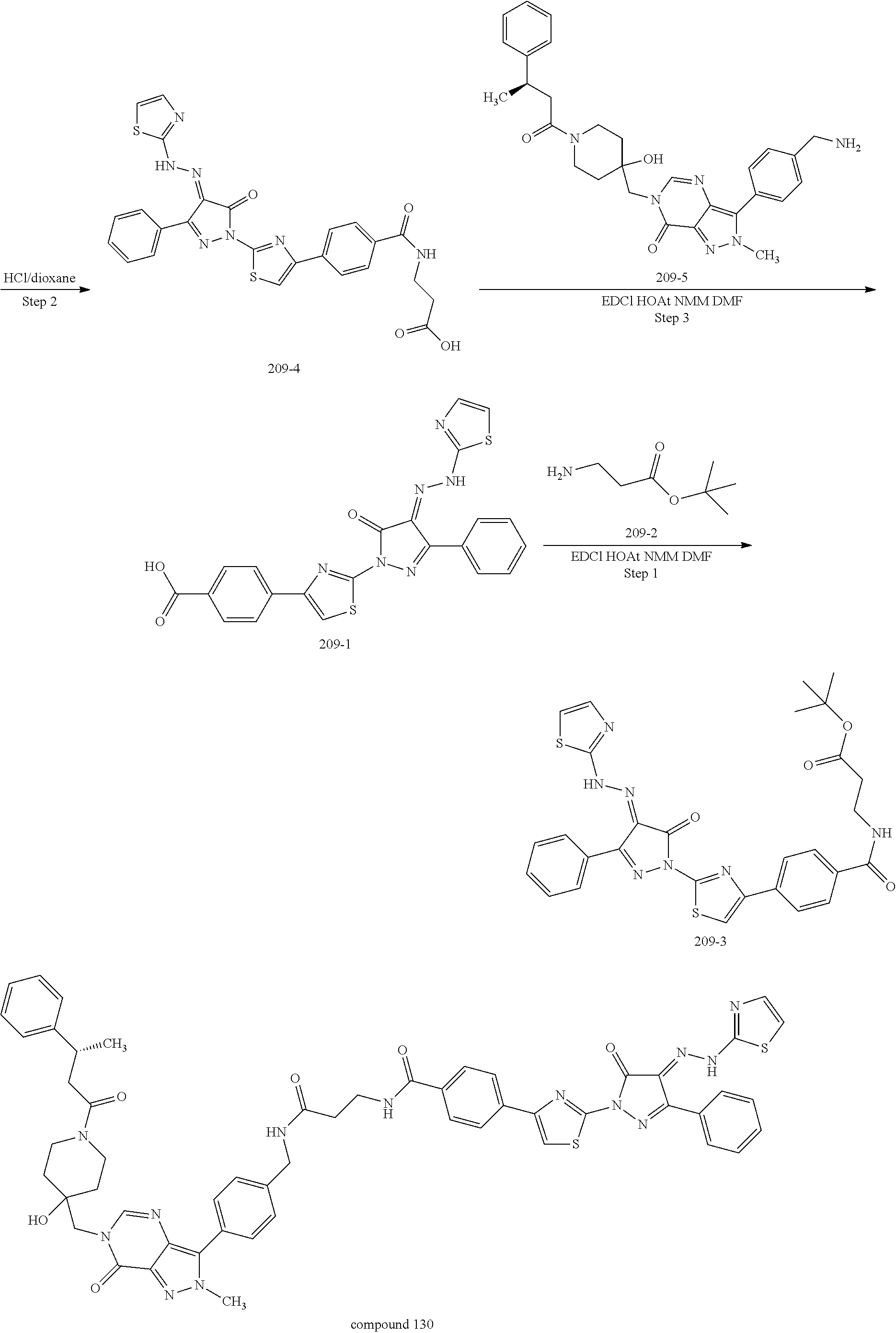

### Step 1: Synthesis of Intermediate 209-3

To a solution of intermediate 209-1 (100 mg, 210.74 umol, 1 eq) and intermediate 209-2 (36.72 mg, 252.89 umol, 1.2 eq) in DMF (0.8 mL) was added EDCI (121.20 mg, 632.23 umol, 3 eq), HOAt (28.68 mg, 210.74 umol, 29.48 uL, 1 eq) and NMM (213.16 mg, 2.11 mmol, 231.69 uL, 10 eq). The mixture was stirred at 25° C. for 1 hr. LC-MS showed Reactant 1 was consumed completely and 65% of desired mass was detected. The reaction was quenched with H2O (5 mL). The mixture was extract with EA (10 mL*3). The combined organic layers dried over Na2SO4, filtered and concentrated under reduced pressure to give a residue. The residue was purified by flash silica gel chromatography (20 g Flash Column, Eluent of 0˜100% DCM/MeOH @ 60 mL/min, DCM:MeOH=20:1, Rf=0.5) and the eluent was concentrated under reduced pressure to give product. Intermediate 209-3 (100 mg, 136.28 umol, 64.67% yield, 82% purity) as orange solid which was confirmed by LCMS.

### Mass Found

LCMS: Retention time: 0.545 min, (M+H)=602.3

LCMS: Retention time: 0.550 min, (M+H)=602.4

### Step 2: Synthesis of Intermediate 209-4

To a solution of intermediate 209-3 (60 mg, 99.72 umol, 1 eq) in dioxane (0.2 mL) was added HCl/dioxane (4 M, 600.00 uL, 24.07 eq). The mixture was stirred at 25° C. for 1 hr. LC-MS showed Reactant 1 was consumed and one major peak with desired mass was detected. The mixture was filtered and concentrated under reduced pressure to give a residue. The crude product was used into the next step without further purification. Intermediate 209-4 (70 mg, crude, HCl) was obtained as a white solid.

### Mass Found

LCMS: Retention time: 0.470 min, (M+H)=546.1

### Step 3: Synthesis of Compound 130

To a solution of intermediate 209-4 (70 mg, 120.26 umol, 1 eq, HCl) and intermediate 209-5 (61.89 mg, 112.31 umol, 9.34e-1 eq, HCl) in DMF (0.8 mL) was added EDCI (115.27 mg, 601.32 umol, 5 eq), HOAt (16.37 mg, 120.26 umol, 16.82 uL, 1 eq), and NMM (121.64 mg, 1.20 mmol, 132.22 uL, 10 eq). The mixture was stirred at 25° C. for 1 hr. LC-MS showed Reactant 1 was consumed and one major peak with desired mass was detected. The mixture was filtered and filter liquor was used to purification. The residue was purified by prep-HPLC (column: Welch Ultimate C18 150*25 mm*5 um; mobile phase: [water (TFA)-ACN]; B %: 40%-70%, 10 min) and the eluent was lyophilized to give product. Compound 130 (33 mg, 26.31 umol, 21.88% yield, 92.185% purity, TFA) as orange solid and confirmed by LCMS, HNMR, FNMR, SFC.

### Mass Found

LCMS: Retention time: 0.503 min, (M+H)=1042.3

LCMS: Retention time: 0.500 min, (M+H)=1042.4

### NMR Data

¹H NMR (400 MHz, DMSO+D2O) δ=8.18-8.10 (m, 2H), 8.05 (d, J=8.4 Hz, 2H), 7.94-7.85 (m, 4H), 7.70-7.68 (m, 1H), 7.59-7.50 (m, 5H), 7.40 (d, J=8.4 Hz, 2H), 7.34 (d, J=4.0 Hz, 1H), 7.27-7.20 (m, 4H), 7.17-7.12 (m, 1H), 4.37 (s, 2H), 4.03 (s, 3H), 4.00-3.75 (m, 4H), 3.57-3.53 (m, 4H), 3.19-3.06 (m, 2H), 2.88-2.81 (m, 1H), 2.55 (s, 2H), 1.40-1.21 (m, 4H), 1.20-1.17 (m, 3H).

### SFC Data

SFC: Retention time: 0.646, OJ-3-MeOH+ CAN (DEA)

### Example 210. Synthesis of [(R,E)-N-(4-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-4-oxobutyl)-4-(2-(5-oxo-3-phenyl-4-(2-(thiazol-2-yl)hydrazono)-4,5-dihydro-1H-pyrazol-1-yl)thiazol-4-yl)benzamide] (Compound 131)

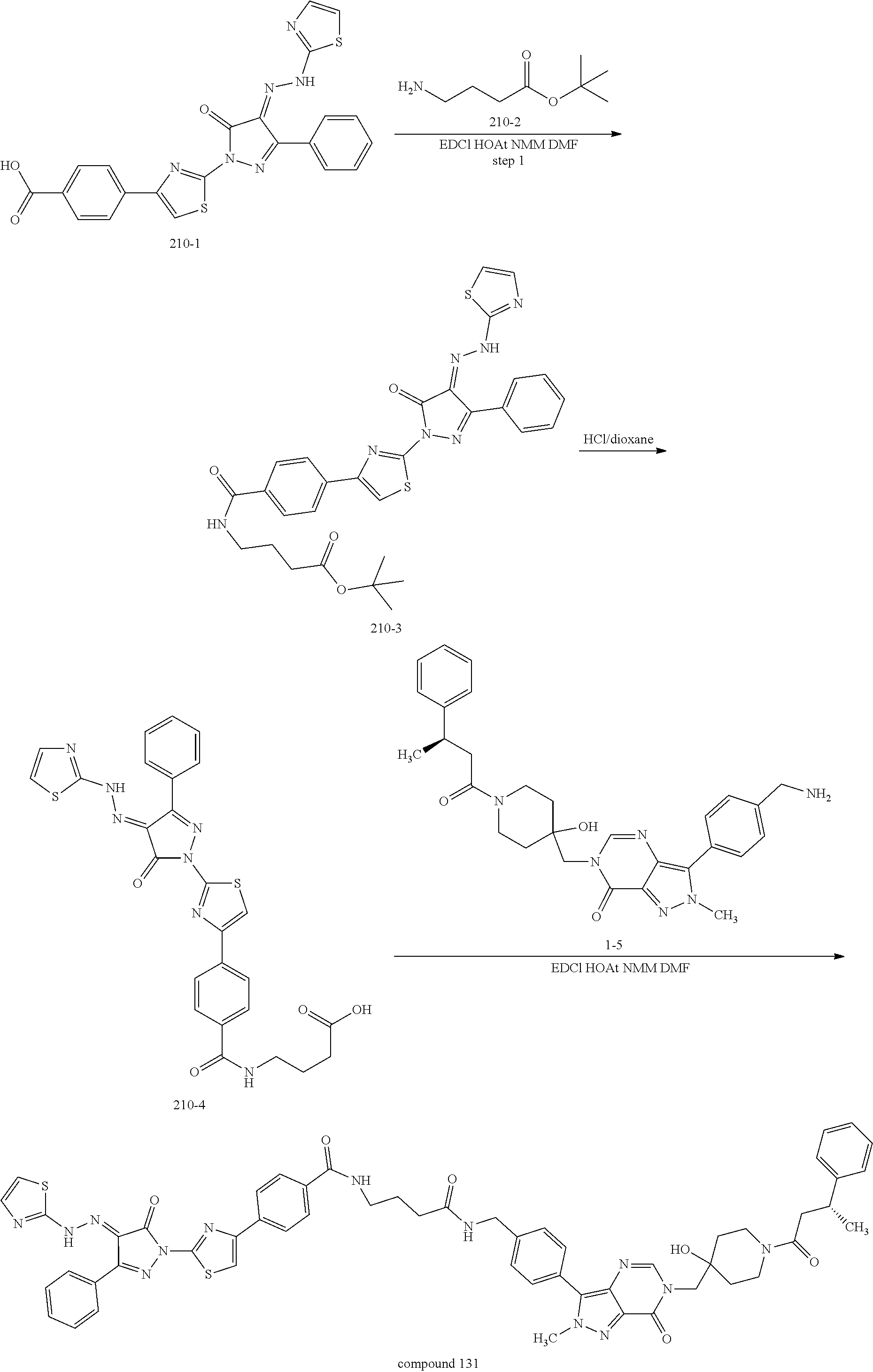 [SMILES: O=C(O)c1ccc(-c2csc(N3N=C(c4ccccc4)/C(=N\Nc4nccs4)C3=O)n2)cc1 ; CC(C)(C)OC(=O)CCCN ; CC(C)(C)OC(=O)CCCNC(=O)c1ccc(-c2csc(N3N=C(c4ccccc4)/C(=N\Nc4nccs4)C3=O)n2)cc1 ; O=C(O)CCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 210-3

Detailed Synthetic Procedure: To a solution of intermediate 210-1 (150 mg, 316.11 umol, 1 eq) in DMF (0.8 mL) was added intermediate 210-2 (92.79 mg, 474.17 μmol, 1.5 eq, HCl), NN (159.87 mg, 1.58 mmol, 173.77 uL, 5 eq), HOAt (43.03 mg, 316.11 μmol, 44.22 uL, 1 eq) and EDCI (181.80 mg, 948.34 μmol, 3 eq). The mixture was stirred at 25° C. for 2 hrs. LC-MS (EC4074-241-P1A2) showed a main peak with desired mass was detected. The mixture was washed with H₂O (5 ml) and extracted with EA 18 ml (3×8 mL). The organic phase was concentrated under reduced pressure to give a white solid, which was purified by column chromatography (SiO₂, MeOH/DCM=0%˜20%) to get intermediate 210-3 (200 mg, crude) was obtained as a red solid which was confirmed by LCMS.

### Mass:

Retention time: 0.551 min, (M+H)=616.2

Retention time: 0.552 min, (M+H)=616.3

### Step 2: Synthesis of Intermediate 1-4

Detailed Synthetic Procedure: To a solution of intermediate 210-3 (200 mg, 324.82 μmol, 1 eq) in HCl/dioxane (2 mL). The mixture was stirred at 25° C. for 2 hrs. LC-MS showed a major peak with mass was detected. The reaction mixture was concentrated under reduced pressure to get intermediate 210-4 (120 mg, 201.32 μmol, 61.98% yield, HCl) was obtained as a red solid.

### Mass:

Retention time: 0.477 min, (M+H)=560.1

### Step 3: Synthesis of Compound 131

Detailed Synthetic Procedure: To a solution of intermediate 210-4 (100 mg, 167.76 μmol, 1 eq, HCl) in DMF (1 mL) was added NN (84.84 mg, 838.80 μmol, 92.22 uL, 5 eq), HOAt (22.83 mg, 167.76 umol, 23.47 uL, 1 eq), intermediate 210-5 (129.50 mg, 251.64 umol, 1.5 eq) and EDCI (96.48 mg, 503.28 umol, 3 eq). The mixture was stirred at 25° C. for 1 hr. LCMS showed desired mass was detected. The mixture was filtered and the filter liquor to give a crude product, which was purified by prep-HPLC (column: Phenomenex luna C18 150*25 mm*10 um; mobile phase: [water(TFA)-ACN];B %: 60%-90%, 1 O0 min). The eluent was concentrated and lyophilized to get Compound 131 (15 mg, 12.54 umol, 7.47% yield, 97.81% purity, TFA) was obtained as a red solid which was confirmed by HNMR, SFC and LCMS.

### Mass Data

Retention time: 0.513 min, (M+H)=1056.2

Retention time: 0.516 min, (M+H)=1056.2

### NMR Data

¹H NMR (400 MHz, DMSO-d6) δ=8.56 (t, J=5.6 Hz, 1H), 8.47 (t, J=6.0 Hz, 1H), 8.16 (d, J=7.2 Hz, 2H), 8.07 (d, J=8.4 Hz, 2H), 7.99-7.93 (m, 4H), 7.72 (d, J=4.0 Hz, 1H), 7.66 (d, J=8.0 Hz, 2H), 7.59-7.52 (m, 3H), 7.45 (d, J=8.4 Hz, 2H), 7.36 (d, J=4.0 Hz, 1H), 7.28-7.23 (m, 4H), 7.18-7.13 (m, 1H), 4.37 (d, J=5.6 Hz, 2H), 4.09 (s, 3H), 4.03-3.98 (m, 2H), 3.32 (d, J=6.0 Hz, 2H), 3.21-3.14 (m, 2H), 2.91-2.85 (m, 1H), 2.57 (d, J=7.2 Hz, 2H), 2.27 (t, J=7.6 Hz, 2H), 1.90-1.81 (m, 2H), 1.52-1.17 (m, 9H).

### SFC Data

SFC: AS-3-MeOH+ACN (DEA)-50-3 mL-35T

### Example 211. Synthesis of [[N-[5-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-5-oxo-pentyl]-4-[2-[(4E)-5-oxo-3-phenyl-4-(thiazol-2-ylhydrazono)pyrazol-1-yl]thiazol-4-yl]benzamide]] (Compound 132)

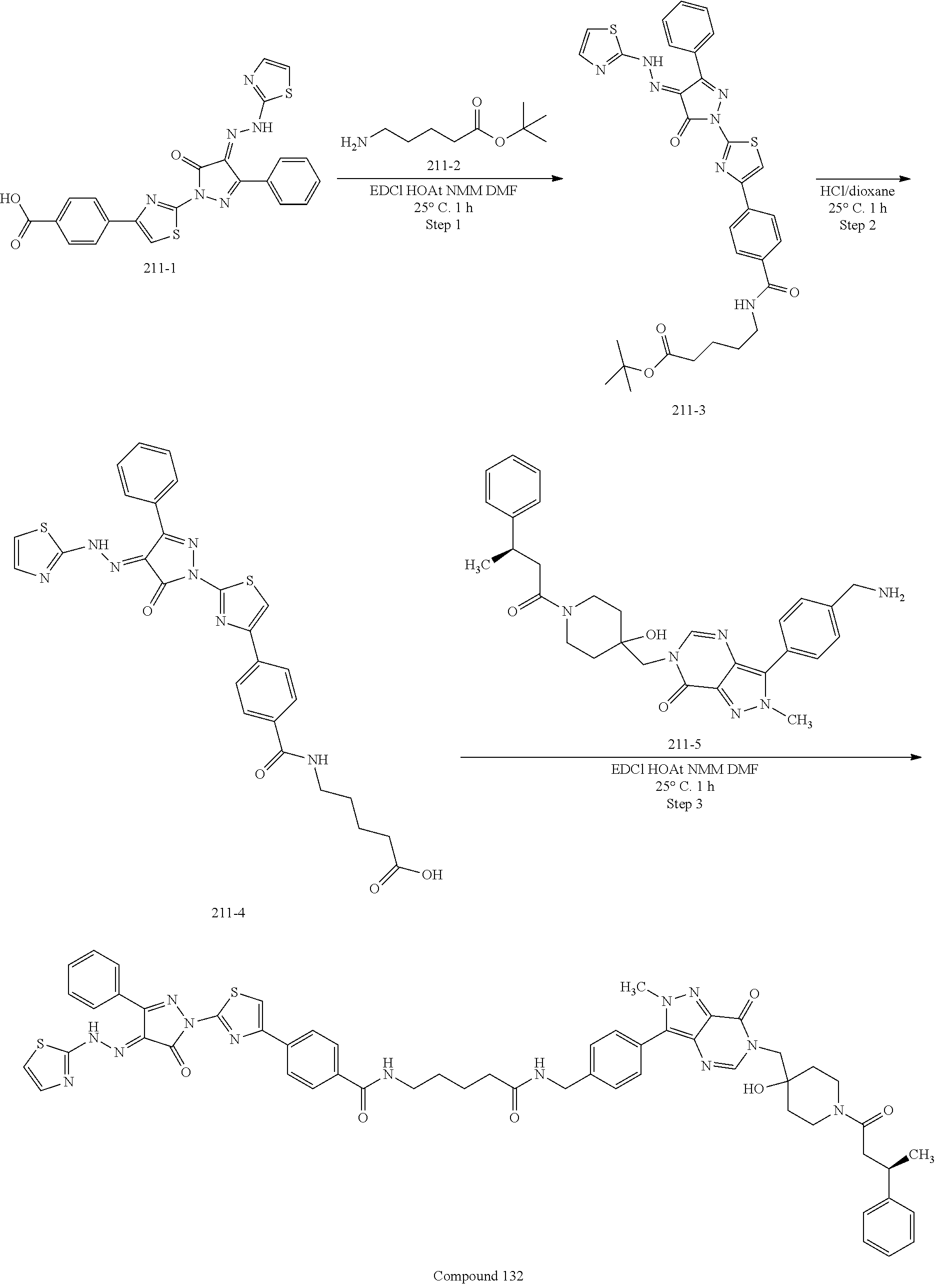 [SMILES: O=C(O)c1ccc(-c2csc(N3N=C(c4ccccc4)/C(=N\Nc4nccs4)C3=O)n2)cc1 ; CC(C)(C)OC(=O)CCCCN ; CC(C)(C)OC(=O)CCCCNC(=O)c1ccc(-c2csc(N3N=C(c4ccccc4)/C(=N\Nc4nccs4)C3=O)n2)cc1 ; O=C(O)CCCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 211-3

Detailed Synthetic Procedure: To a solution of intermediate 211-1 (100 mg, 210.74 umol, 1 eq) in DMF (1 mL) was added EDCI (202.00 mg, 1.05 mmol, 5 eq), HOAt (57.37 mg, 421.48 umol, 58.96 uL, 2 eq) and NMM (213.16 mg, 2.11 mmol, 231.69 uL, 10 eq). Then the mixture was added intermediate 211-2 (36.51 mg, 210.74 umol, 1 eq), which was stirred at 25° C. for 1 hr. The LCMS showed desired mass was detected. TLC (DCM/MeOH=10:1) indicated Reactant 1 (Rt=0.1) was remained and product one new spot (Rt=0.4) formed. The reaction was clean according to TLC. The mixture was pour into water (3 mL) and extracted with EA (5 mL*3). The combined organic layers were dried over Na2SO4, filtered and concentrated under reduced pressure to give a residue. The residue was purified by flash silica gel chromatography (20 g Silica Flash Column, Eluent of 0-100% DCM/MeOH @ 30 mL/min), which was concentrated under reduced pressure to give intermediate 211-3 (150 mg, crude) was obtained as yellow solid and confirmed by LCMS.

### Mass Found

LCMS: Retention time: 0.557 min, (M+H)=630.5

LCMS: Retention time: 0.558 min, (M+H)=630.5

### Step 2: Synthesis of Intermediate 211-4

Detailed Synthetic Procedure: To a solution of intermediate 211-3 (100 mg, 158.79 umol, 1 eq) in HCl/dioxane (0.7 mL) and DCM (0.3 mL), The mixture was stirred at 25° C. for 1 hr. The LCMS showed desired mass was detected. The mixture was filtered and concentrated under reduced pressure to give a residue. The residue without purification and it was concentrated under reduced pressure to give intermediate 211-4 (150 mg, crude) was obtained as white solid by LCMS.

### Mass Found

LCMS: Retention time: 0.483 min, (M+H)=574.5

LCMS: Retention time: 0.481 min, (M+H)=574.5

### Step 3: Synthesis of Compound 132

Detailed Synthetic Procedure: To a solution of intermediate 211-4 (50 mg, 87.16 umol, 1.2 eq) and intermediate 211-5 (37.38 mg, 72.63 umol, 1 eq) in DMF (0.5 mL) was added HOAt (19.77 mg, 145.27 umol, 20.32 uL, 2 eq) and NN (73.47 mg, 726.35 umol, 79.86 uL, 10 eq) and EDCI (69.62 mg, 363.17 umol, 5 eq). The mixture was stirred at 25° C. for 1 hr. The LCMS showed desired mass was detected. The mixture was filtered and concentrated under reduced pressure to give a residue. The residue was purified by prep-HPLC (column: Phenomenex luna C18 150*25 mm*10 um;mobile phase: [water(TFA)-ACN];B %: 40%-70%, 10 min) and the eluent was lyophilizated to give Compound 132 (15 mg, 11.95 umol, 16.46% yield, 94.384% purity, TFA) was obtained as orange solid by LCMS, SFC, FNMR and HNMR.

### Mass Found

LCMS: Retention time: 0.521 min, (M+H)=1071.2

LCMS: Retention time: 0.512 min, (M+H)=1071.4

SFC data

SFC: Retention time: 0.568 min, AS-3-IPA+ACN(DEA)-60-3 mL-35T

### NMR Data

¹H NMR (400 MHz, DMSO-d6) δ=8.54 (t, J=5.6 Hz, 1H), 8.43 (t, J=6.4 Hz, 1H), 8.16 (d, J=7.2 Hz, 2H), 8.06 (d, J=8.4 Hz, 2H), 7.99-7.90 (m, 4H), 7.72 (d, J=4.0 Hz, 1H), 7.65 (d, J=8.4 Hz, 2H), 7.58-7.51 (m, 3H), 7.44 (d, J=8.4 Hz, 2H), 7.35 (d, J=4.0 Hz, 1H), 7.29-7.22 (m, 4H), 7.19-7.11 (m, 1H), 4.36 (d, J=5.2 Hz, 2H), 4.08 (s, 3H), 4.03-3.88 (m, 4H), 3.33-3.26 (m, 3H), 3.20-3.13 (m, 2H), 2.88-2.86 (m, 1H), 2.60-2.54 (m, 3H), 2.23 (t, J=6.8 Hz, 2H), 1.66-1.53 (m, 4H), 1.49-1.22 (m, 4H), 1.20 (d, J=7.2 Hz, 3H).

### Example 212. Synthesis of [N-[6-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-6-oxo-hexyl]-4-[2-[(4E)-5-oxo-3-phenyl-4-(thiazol-2-ylhydrazono)pyrazol-1-yl]thiazol-4-yl]benzamide] (Compound 133)

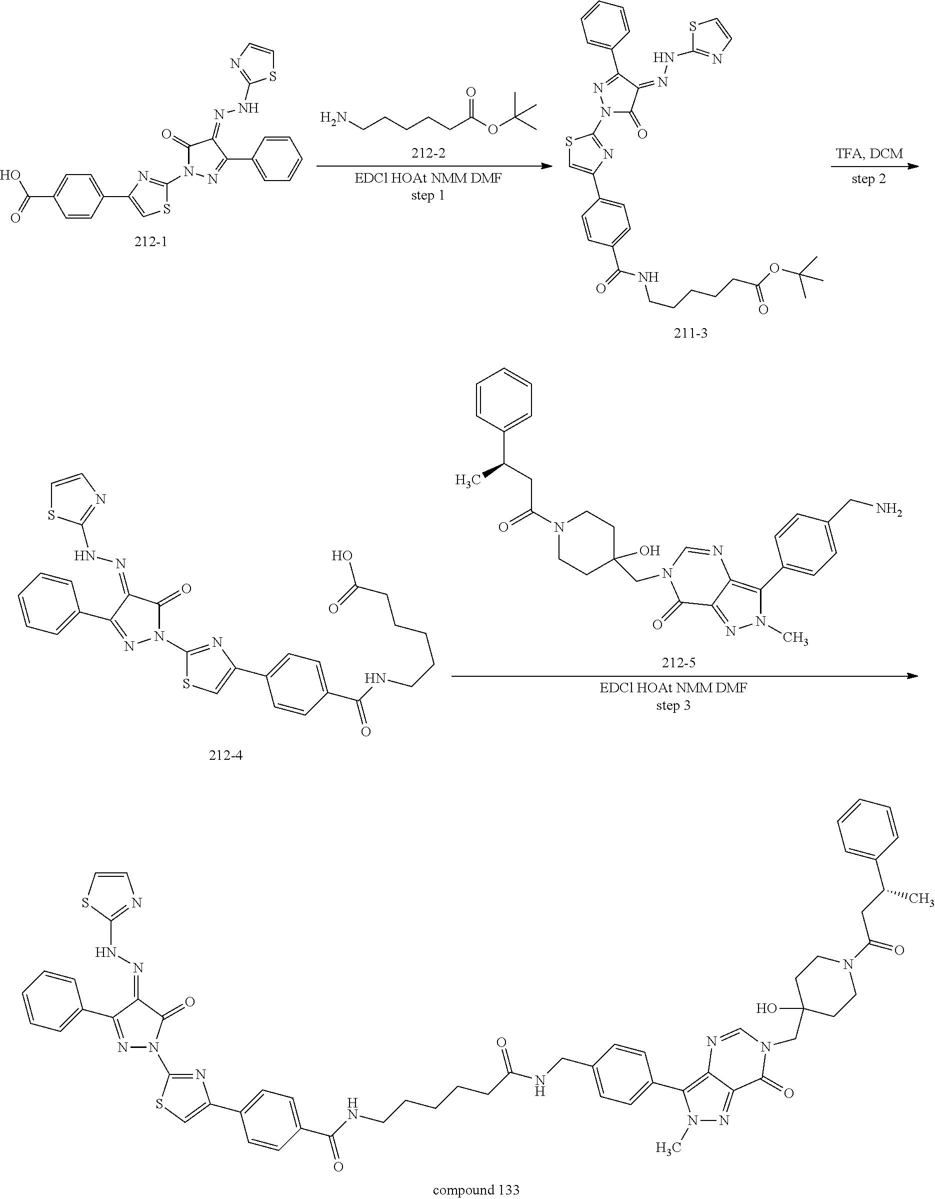

### Step 1: Synthesis of Intermediate 212-3

To a solution of intermediate 212-1 (80 mg, 168.59 umol, 1 eq) in DMF (0.8 mL) was added EDCI (96.96 mg, 505.78 umol, 3 eq), NMM (85.26 mg, 842.97 umol, 92.68 uL, 5 eq) and HOAt (22.95 mg, 168.59 umol, 23.58 uL, 1 eq). The mixture was stirred at 25° C. for 15 min. Then the intermediate 212-2 (37.89 mg, 202.31 umol, 1.2 eq) was added into the mixture, the mixture was stirred at 25° C. for 2 hrs. LCMS showed 81% of desired mass was detected. TLC (SiO2, by UV 254 nm, DCM/MeOH=10:1, Rt=0.5), TLC (SiO2, by UV 254 nm, EA=1, Rf=0.3). The reaction mixture was washed with H2O (2 mL) and mixture was extracted with EA 9 ml (3 ml*3), and combined organic phase was dried with anhydrous sodium sulfate, filtered and concentrated to give crude product. The crude product was purified by column chromatography (SiO2, DCM/MeOH=1/0 to 0/1) and the eluent was concentrated under reduced pressure to give Intermediate 212-3 (120 mg, 124.89 umol, 74.08% yield, 67% purity) as a brown oil which was confirmed by LCMS

### Mass Found

LCMS: Retention time: 0.576 min, (M+H)=644.5

LCMS: Retention time: 0.581 min, (M+H)=644.5

### Step 2: Synthesis of Intermediate 212-4

Detailed Synthetic Procedure: To a solution of intermediate 212-3 (60 mg, 93.20 umol, 1 eq) in DCM (0.6 mL) was added TFA (308.00 mg, 2.70 mmol, 0.2 mL, 28.98 eq). The mixture was stirred at 25° C. for 1 hr. LCMS showed 70% of desired mass was detected. Filtered and concentrated under reduced pressure to give intermediate 212-4 (60 mg, crude, TFA) as a red oil.

### Mass Found

LCMS: Retention time: 0.491 min, (M+H)=588.3

### Step 3: Synthesis of Compound 133

Detailed Synthetic Procedure: To a solution of intermediate 212-4 (60 mg, 102.10 umol, 1 eq) in DMF (0.5 mL) was added EDCI (58.72 mg, 306.29 umol, 3 eq), NMM (51.63 mg, 510.49 umol, 56.12 uL, 5 eq) and HOAt (13.90 mg, 102.10 umol, 14.28 uL, 1 eq), the mixture was stirred at 25° C. for 15 min. Then the intermediate 212-5 (42.03 mg, 81.68 umol, 0.8 eq) was added into the mixture and stirred at 25° C. for 2 hrs. LCMS showed 42% of desired mass was detected. The mixture was diluted with MeOH (1 ml) and purified by prep-HPLC directly (column: Phenomenex Luna C18 150*25 mm*10 um;mobile phase: [water(TFA)-ACN];B %: 40%-70%, 10 min) and the eluent was lyophilized to give Compound 133 (8.79 mg, 7.03 umol, 6.89% yield, 95.882% purity, TFA) as orange solid was confirmed by HNMR, SFC and LCMS.

### Mass Found

LCMS: Retention time: 0.525 min, (M+H)=1084.2

LCMS: Retention time: 0.525 min, (M+H)=1084.3

### NMR Data

¹H NMR (400 MHz, DMSO+D2O) δ=8.16 (d, J=7.6 Hz, 2H), 8.06 (d, J=8.4 Hz, 2H), 7.97-7.90 (m, 4H), 7.71 (d, J=4.0 Hz, 1H), 7.64 (d, J=8.0 Hz, 2H), 7.57-7.52 (m, 3H), 7.43 (d, J=8.0 Hz, 2H), 7.35 (d, J=4.0 Hz, 1H), 7.26-7.22 (m, 4H), 7.17-7.13 (m, 1H), 4.35 (s, 2H), 4.08 (s, 3H), 4.02-3.95 (m, 2H), 3.89 (d, J=7.2 Hz, 1H), 3.69-3.55 (m, 2H), 3.30-3.26 (m, 2H), 3.18-3.13 (m, 1H), 2.91-2.82 (m, 1H), 2.61-2.57 (m, 2H), 2.22-2.18 (m, 2H), 1.61-1.54 (m, 4H), 1.38-1.32 (m, 4H), 1.24-1.18 (m, 5H)

### SFC Found

SFC: Retention time: 0.769 min, AS-3-MeOH+CAN (DEA)-50-3 mL-35T

### Example 213. Synthesis of [N-[7-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-7-oxo-heptyl]-4-[2-[(4E)-5-oxo-3-phenyl-4-(thiazol-2-ylhydrazono)pyrazol-1-yl]thiazol-4-yl]benzamide] (Compound 134)

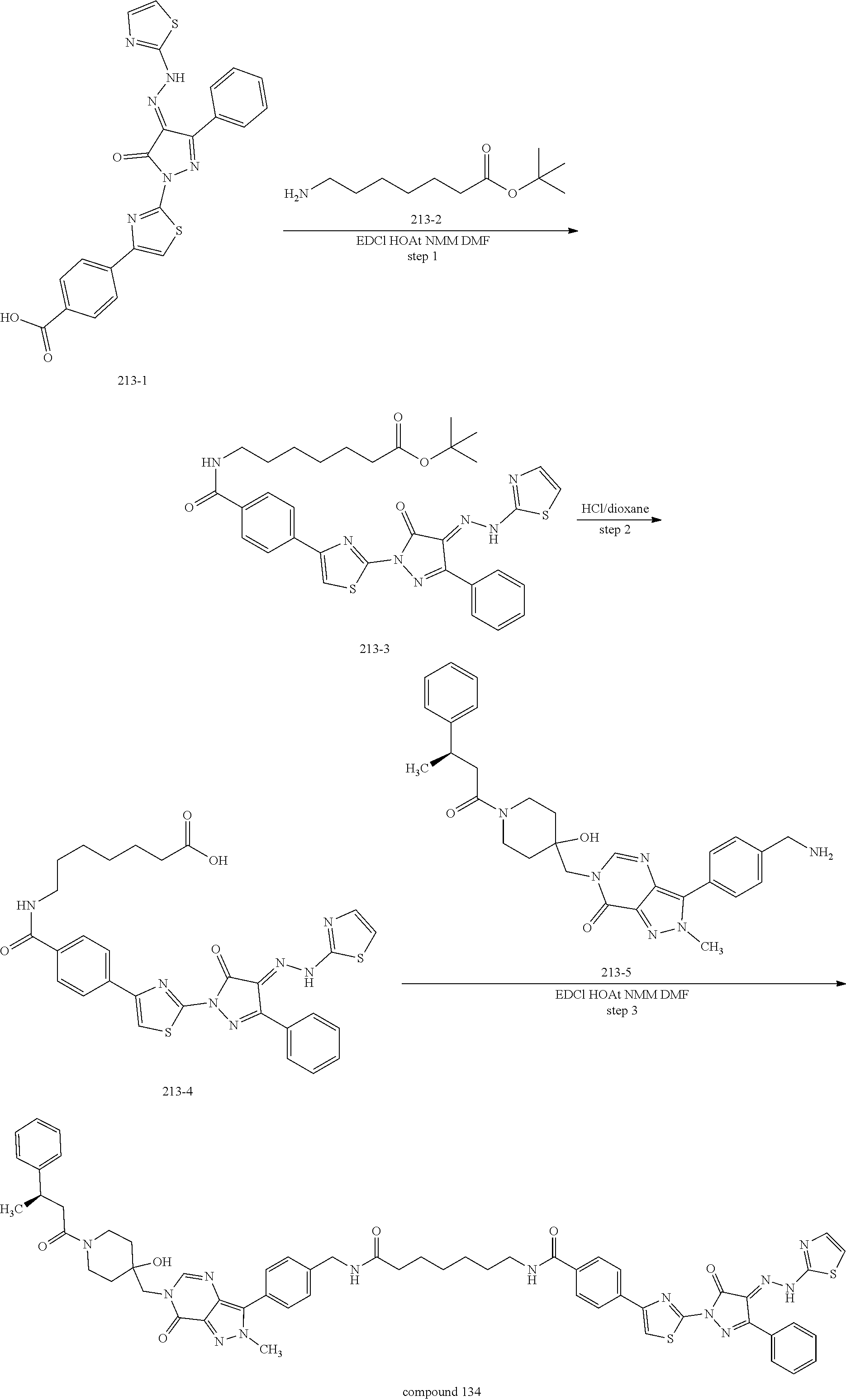 [SMILES: O=C(O)c1ccc(-c2csc(N3N=C(c4ccccc4)/C(=N\Nc4nccs4)C3=O)n2)cc1 ; CC(C)(C)OC(=O)CCCCCCN ; CC(C)(C)OC(=O)CCCCCCNC(=O)c1ccc(-c2csc(N3N=C(c4ccccc4)/C(=N\Nc4nccs4)C3=O)n2)cc1 ; O=C(O)CCCCCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCCCCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 213-3

To a solution of intermediate 213-1 (80 mg, 168.59 umol, 1 eq) in DMF (0.8 mL) was added EDCI (96.96 mg, 505.78 umol, 3 eq), NMM (85.26 mg, 842.97 umol, 92.68 uL, 5 eq) and HOAt (22.95 mg, 168.59 umol, 23.58 uL, 1 eq). The mixture was stirred at 25° C. for 15 min. Then the intermediate 213-2 (40.73 mg, 202.31 umol, 1.2 eq) was added into the mixture and stirred at 25° C. for 2 hrs. LCMS showed 85% of desired mass was detected. TLC (SiO2, by UV 254 nm, DCM/MeOH=10:1, Rt=0.5). The reaction mixture was washed with H2O (2 mL) at 25° C. and the mixture was extracted with EA 9 ml (3 ml*3), and combined organic phase was dried with anhydrous sodium sulfate, filtered and concentrated to give crude product. The crude product was purified by column chromatography (SiO2, DCM/MeOH=1/0 to 0/1) and the eluent was concentrated under reduced pressure to give intermediate 213-3 (90 mg, 99.88 umol, 59.24% yield, 73% purity) as brown oil and confirmed by LCMS.

### Mass Found

LCMS: Retention time: 0.588 min, (M+H)=658.5

LCMS: Retention time: 0.591 min, (M+H)=658.6

### Step 2: Synthesis of Intermediate 213-4

Detailed Synthetic Procedure: To a solution of intermediate 213-3 (40 mg, 60.81 umol, 1 eq) in DCM (0.6 mL) was added TFA (6.93 mg, 60.81 umol, 4.50 uL, 1 eq). The mixture was stirred at 25° C. for 1 hr. LCMS showed 72% of desired mass was detected. The mixture was concentrated under reduced pressure to give intermediate 213-4 (40 mg, crude, TFA) as a red oil.

### Mass Found

LCMS: Retention time: 0.510 min, (M+H)=602.4

### Step 3: Synthesis of Compound 134

Detailed Synthetic Procedure: To a solution of intermediate 213-4 (40 mg, 66.48 umol, 1 eq) in DMF (0.5 mL) was added EDCI (38.23 mg, 199.44 umol, 3 eq), NMM (33.62 mg, 332.39 umol, 36.54 uL, 5 eq) and HOAt (9.05 mg, 66.48 umol, 9.30 uL, 1 eq), the mixture was stirred at 25° C. for 15 min. Then the intermediate 213-5 (27.37 mg, 53.18 umol, 0.8 eq) was added into the mixture and stirred at 25° C. for 2 hrs. LCMS showed 53% of desired mass was detected. The mixture was diluted with MeOH (1 ml) and purified by prep-HPLC directly (column: Phenomenex luna C18 150*25 mm*10 um;mobile phase: [water(TFA)-ACN];B %: 42%-72%, 10 min) and the eluent was lyophilized to give Compound 134 (6.59 mg, 5.34 umol, 8.04% yield, 98.298% purity, TFA) as orange solid which was confirmed by HNMR, SFC, and LCMS.

### Mass Found

LCMS: Retention time: 0.533 min, (M+H)=1098.2

LCMS: Retention time: 0.537 min, (M+H)=1098.3

### NMR Data

¹H NMR (400 MHz, DMSO+D2O) δ=8.15 (d, J=6.8 Hz, 2H), 8.06 (d, J=8.4 Hz, 2H), 7.95-7.88 (m, 4H), 7.69 (d, J=4.0 Hz, 1H), 7.64 (d, J=8.4 Hz, 2H), 7.57-7.52 (m, 3H), 7.43 (d, J=8.4 Hz, 2H), 7.34 (d, J=4.0 Hz, 1H), 7.26-7.22 (m, 4H), 7.16-7.12 (m, 1H), 4.34 (s, 1H), 4.38-4.31 (m, 2H), 4.07 (s, 3H), 4.01-3.94 (m, 2H), 3.87 (d, J=10.4 Hz, 1H), 3.55 (d, J=2.6 Hz, 2H), 3.27-3.24 (m, 2H), 3.16-3.11 (m, 1H), 2.90-2.78 (m, 1H), 2.61-2.56 (m, 2H), 2.19-2.16 (m, 2H), 1.58-1.52 (m, 4H), 1.36-1.28 (m, 7H), 1.22-1.17 (m, 4H).

### SFC Found

SFC: Retention time: 0.870 min, AS-3-MeOH+ACN (DEA)-50-3 mL-35T

### Example 214 Synthesis of N-[8-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-8-oxo-octyl]-4-[2-[(4E)-5-oxo-3-phenyl-4-(thiazol-2-ylhydrazono)pyrazol-1-yl]thiazol-4-yl]benzamide (Compound 135)

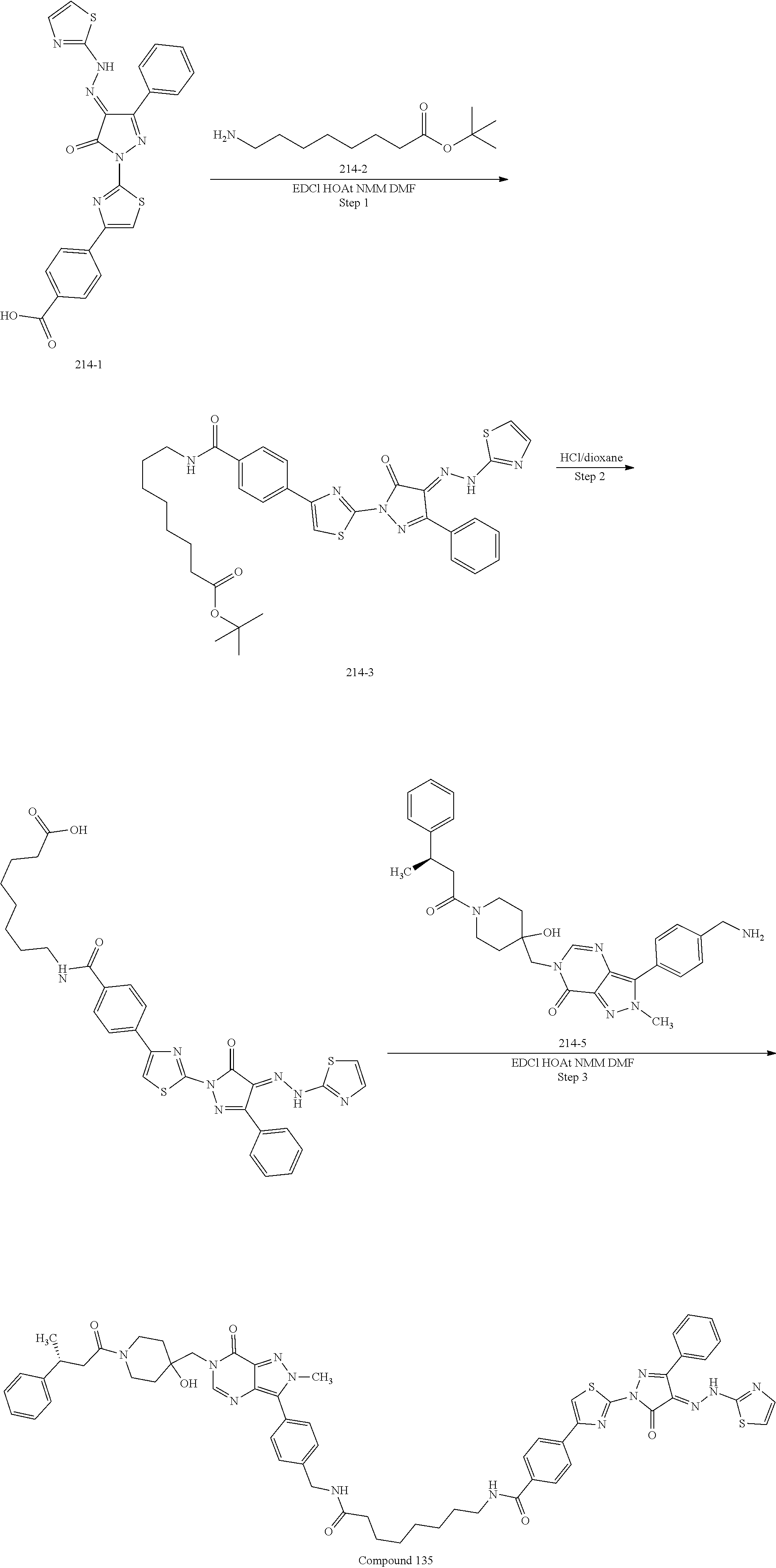 [SMILES: O=C(O)c1ccc(-c2csc(N3N=C(c4ccccc4)/C(=N\Nc4nccs4)C3=O)n2)cc1 ; CC(C)(C)OC(=O)CCCCCCCN ; CC(C)(C)OC(=O)CCCCCCCNC(=O)c1ccc(-c2csc(N3N=C(c4ccccc4)/C(=N\Nc4nccs4)C3=O)n2)cc1 ; O=C(O)CCCCCCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCCCCCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 214-3

Detailed Synthetic Procedure: To a solution of intermediate 214-1 and intermediate 214-2 in DMF (1 mL) was added EDCI (121.20 mg, 632.23 umol, 3 eq), NMM (106.58 mg, 1.05 mmol, 115.85 uL, 5 eq) and HOAt (28.68 mg, 210.74 umol, 29.48 uL, 1 eq). The mixture was stirred at 25° C. for 1 hr. LCMS showed SM was consumed completely and 59% of desired mass was detected. The reaction mixture was diluted with water (15 mL) and extracted with DCM (20 mL*3). The combined organic layer was dried over Na2SO4, filtered and concentrated in vacuo to give a residue. The residue was purified by column chromatography (SiO₂, PE:EA=1:1 to DCM:MeOH=10:1), then the organic liquid was concentrated in vacuo to give intermediate 214-3 (150 mg, 207.64 umol, 98.53% yield, 93% purity) as a orange solid which was confirmed by LCMS and HNMR.

### Mass Found

Retention time=0.605 min, (M+H)=673.0

Retention time=0.606 min, (M+H)=672.5

### NMR Data

¹H NMR (400 MHz, DMSO-d6) δ=8.48-8.46 (m, 1H), 8.20 (br d, J=7.6 Hz, 1H), 8.06 (d, J=8.4 Hz, 1H), 7.95-7.89 (m, 3H), 7.82 (s, 1H), 7.63 (d, J=3.6 Hz, 1H), 7.49 (s, 1H), 7.46-7.40 (m, 1H), 7.24 (d, J=3.6 Hz, 1H), 5.75 (s, 1H), 3.30-3.22 (m, 3H), 2.17-2.15 (m, 2H), 1.55-1.45 (m, 4H), 1.39-1.36 (m, 9H), 1.29 (br s, 4H), 1.24-1.13 (m, 2H).

### Step 2: Synthesis of Intermediate 214-4

Detailed Synthetic Procedure: To a solution of intermediate 214-3 (100 mg, 148.85 umol, 1 eq) in dioxane (1 mL) was added HCl/dioxane (4 M, 37.21 uL, 1 eq). The mixture was stirred at 25° C. for 0.5 hr. LCMS (EC5839-45-P1A6) showed SM was consumed completely and 87% of desired mass was detected. The reaction mixture was concentrated in vacuo to give intermediate 214-4 (100 mg, 141.30 umol, 94.93% yield, 87% purity) as a white solid which was confirmed by LCMS.

### Mass:

Retention time=0.522 min, (M+H)=616.5

Retention time=0.515 min, (M+H)=616.5

### Step 3: Synthesis of Compound 135

Detailed Synthetic Procedure: To a solution of intermediate 214-4 (70 mg, 113.69 umol, 1 eq) and intermediate 214-5 (70.21 mg, 136.42 umol, 1.2 eq) in DMF (2 mL) was added EDCI (65.38 mg, 341.06 umol, 3 eq), NMM (57.50 mg, 568.44 umol, 62.50 uL, 5 eq) and HOAt (15.47 mg, 113.69 umol, 15.90 uL, 1 eq). The mixture was stirred at 25° C. for 1 hr. LCMS showed SM was consumed completely and 61% of desired mass was detected. The mixture was purified by prep-HPLC (column: Phenomenex luna C18 150*25 mm*10 um;mobile phase: [water(TFA)-ACN];B %: 45%-75%, 10 min) to give a residue. Then residue was concentrated in vacuo and lyophilized to give Compound 135 (24.03 mg, 21.17 umol, 18.62% yield, 98% purity) as a orange solid which was confirmed by LCMS, SFC, and HNMR.

### Mass:

Retention time=0.551 min, (M+H)=1112.3

Retention time=0.543 min, (M+H)=1112.3

### NMR Data:

¹H NMR (400 MHz, DMSO-d6) δ=8.50-8.48 (m, 1H), 8.40-8.38 (m, 1H), 8.16 (br d, J=6.8 Hz, 2H), 8.06 (d, J=8.4 Hz, 2H), 7.98-7.95 (m, 2H), 7.92 (d, J=8.4 Hz, 2H), 7.73 (d, J=4.0 Hz, 1H), 7.65 (d, J=8.0 Hz, 2H), 7.62-7.49 (m, 4H), 7.43 (d, J=8.0 Hz, 2H), 7.36 (d, J=4.0 Hz, 1H), 7.27-7.23 (m, 4H), 7.17-7.13 (m, 1H), 4.35 (br d, J=5.6 Hz, 2H), 4.09 (s, 3H), 4.01-3.95 (m, 2H), 3.95-3.88 (m, 2H), 3.70-3.58 (m, 2H), 3.29-3.25 (m, 2H), 3.20-3.11 (m, 2H), 2.91-2.83 (m, 1H), 2.57 (br d, J=6.8 Hz, 1H), 2.17-2.16 (m, 2H), 1.58-1.52 (m, 4H), 1.37 (br s, 2H), 1.32 (br s, 6H), 1.25-1.18 (m, 5H).

### SFC Data:

Retention time: 0.887 min, AS-3-IPA+ACN(DEA)-60-3 mL-35T

### Example 215. Synthesis of N-[9-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-9-oxo-nonyl]-4-[2-[(4E)-5-oxo-3-phenyl-4-(thiazol-2-ylhydrazono)pyrazol-1-yl]thiazol-4-yl]benzamide (Compound 136)

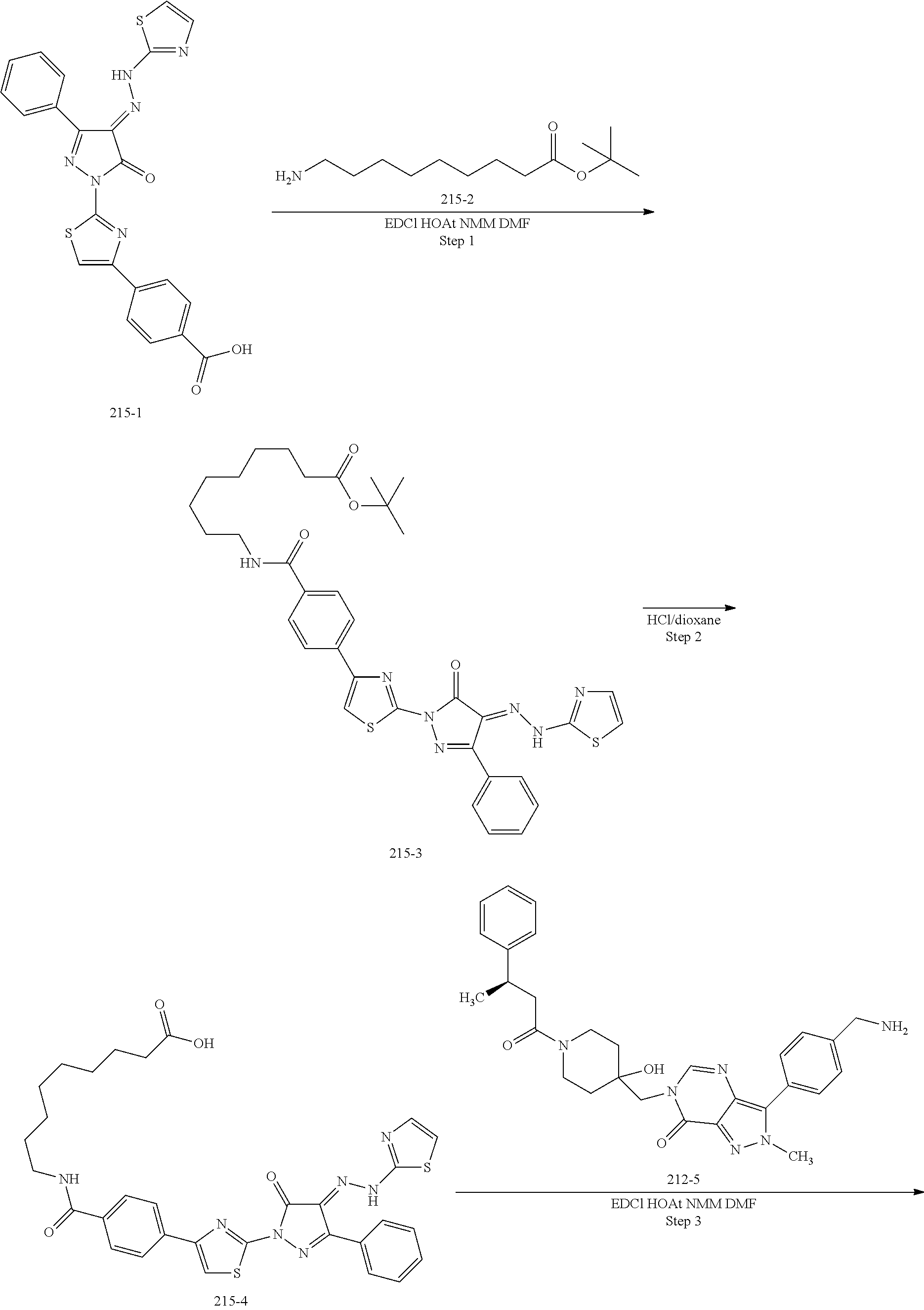 [SMILES: O=C(O)c1ccc(-c2csc(N3N=C(c4ccccc4)/C(=N\Nc4nccs4)C3=O)n2)cc1 ; CC(C)(C)OC(=O)CCCCCCCCN ; CC(C)(C)OC(=O)CCCCCCCCNC(=O)c1ccc(-c2csc(N3N=C(c4ccccc4)/C(=N\Nc4nccs4)C3=O)n2)cc1 ; O=C(O)CCCCCCCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1]

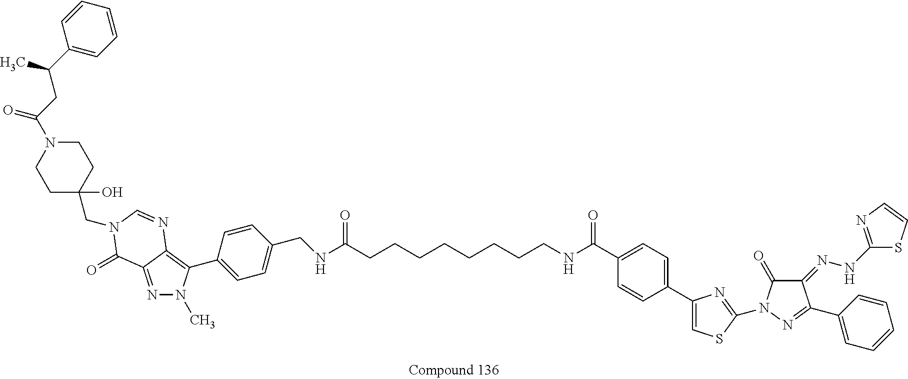 [SMILES: C[C@H](CC(=O)N1CCC(O)(Cn2cnc3c(-c4ccc(CNC(=O)CCCCCCCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 215-3

Detailed Synthetic Procedure: To a solution of intermediate 215-1 (100 mg, 210.74 umol, 1 eq) and intermediate 215-2 (62.84 mg, 273.96 umol, 1.3 eq) in DMF (1 mL) was added EDCI (121.20 mg, 632.23 umol, 3 eq), NMM (106.58 mg, 1.05 mmol, 115.85 uL, 5 eq) and HOAt (28.68 mg, 210.74 umol, 29.48 uL, 1 eq). The mixture was stirred at 25° C. for 1 hr. LCMS showed SM was consumed completely and 64% of desired mass was detected. The reaction mixture was diluted with water (15 mL) and extracted with DCM (20 mL*3). The combined organic layer was dried over Na₂SO₄, filtered and concentrated in vacuo to give a residue. The residue was purified by column chromatography (SiO₂, PE:EA=1:1 to DCM:MeOH=10:1), then the organic liquid was concentrated in vacuo to give intermediate 215-3 (150 mg, 201.21 umol, 95.48% yield, 92% purity) as a orange solid which was confirmed by LCMS and HNMR.

### Mass Found

Retention time=0.620 min, (M+H)=686.5

Retention time=0.624 min, (M+H)+23=708.6

### NMR Data

¹H NMR (400 MHz, DMSO-d6) δ=8.48-8.46 (m, 1H), 8.21 (br d, J=6.8 Hz, 1H), 8.07 (d, J=8.4 Hz, 1H), 7.95-7.90 (m, 3H), 7.82 (s, 1H), 7.64 (br d, J=3.6 Hz, 1H), 7.54-7.46 (m, 2H), 7.45-7.40 (m, 1H), 7.24 (d, J=3.6 Hz, 1H), 3.26 (br d, J=6.4 Hz, 2H), 2.33-2.32 (m, 2H), 1.55-1.46 (m, 4H), 1.39 (s, 9H), 1.33-1.24 (m, 9H).

### Step 2: Synthesis of Intermediate 215-4

Detailed Synthetic Procedure: To a solution of intermediate 215-3 (100 mg, 145.80 umol, 1 eq) in DCM (0.5 mL) was added HCl/dioxane (4 M, 36.45 uL, 1 eq). The mixture was stirred at 25° C. for 1 hr. LCMS (EC5839-49-P1A1) showed SM was consumed completely and 89% of desired mass was detected. The reaction mixture was concentrated in vacuo to give intermediate 215-4 (100 mg, 141.33 umol, 96.93% yield, 89% purity) as a orange solid.

### Mass:

Retention time=0.532 min, (M+H)=630.3.

### Step 3: Synthesis of Compound 136

Detailed Synthetic Procedure: To a solution of intermediate 215-4 (100 mg, 158.79 umol, 1 eq) and intermediate 215-5 (81.72 mg, 158.79 umol, 1 eq) in DMF (2 mL) was added EDCI (91.32 mg, 476.38 umol, 3 eq), NMM (80.31 mg, 793.96 umol, 87.29 uL, 5 eq) and HOAt (21.61 mg, 158.79 umol, 22.21 uL, 1 eq). The mixture was stirred at 25° C. for 1 hr. LCMS showed SM was consumed completely and 88% of desired mass was detected. The mixture was diluted with MeOH (2 ml) and purified by prep-HPLC directly (column: Phenomenex luna C18 150*25 mm*10 um;mobile phase: [water(TFA)-ACN];B %: 48%-78%, 10 min) to give a residue. Then residue was concentrated in vacuo and lyophilized to give Compound 136 (39.79 mg, 35.33 umol, 22.25% yield, 100% purity) as a orange solid which was confirmed by LCMS, SFC and HNMR.

### Mass:

Retention time=0.561 min, (M+H)=1126.4

Retention time=0.558 min, (M+H)=1126.3

### NMR Data:

¹H NMR (400 MHz, DMSO-d6) δ=8.49-8.48 (m, 1H), 8.38-8.37 (m, 1H), 8.16 (br d, J=6.8 Hz, 2H), 8.06 (d, J=8.4 Hz, 2H), 7.97-7.94 (m, 2H), 7.91 (d, J=8.4 Hz, 2H), 7.72 (d, J=4.0 Hz, 1H), 7.65 (d, J=8.4 Hz, 2H), 7.57-7.52 (m, 3H), 7.42 (d, J=8.4 Hz, 2H), 7.35 (d, J=4.0 Hz, 1H), 7.26-7.22 (m, 4H), 7.17-7.13 (m, 1H), 4.34 (br d, J=4.8 Hz, 2H), 4.08 (s, 3H), 4.01-3.95 (m, 2H), 3.89 (br d, J=5.6 Hz, 2H), 3.69-3.58 (m, 2H), 3.28-3.24 (m, 2H), 3.20-3.12 (m, 2H), 2.90-2.83 (m, 1H), 2.56 (br d, J=7.6 Hz, 2H), 2.16-2.14 (m, J=7.4 Hz, 2H), 1.60-1.44 (m, 6H), 1.30 (br s, 10H), 1.19 (d, J=7.2 Hz, 3H).

### SFC Data:

Retention time: 1.030 min, AS-3-IPA+ACN(DEA)-60-3 mL-35T.

### Example 216. Synthesis of N-[9-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-9-oxo-nonyl]-4-[2-[(4E)-5-oxo-3-phenyl-4-(thiazol-2-ylhydrazono)pyrazol-1-yl]thiazol-4-yl]benzamide (Compound 137)

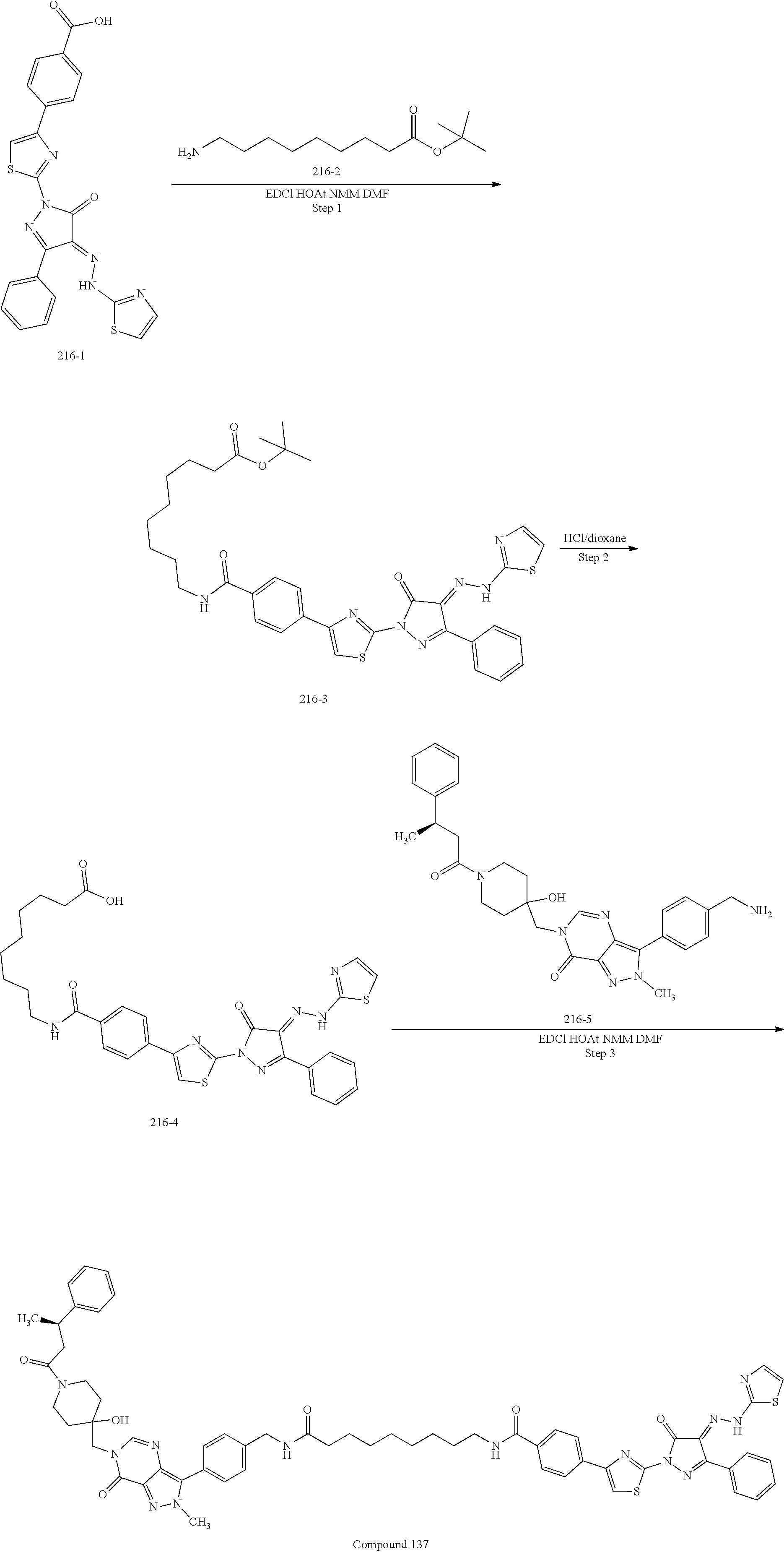 [SMILES: O=C(O)c1ccc(-c2csc(N3N=C(c4ccccc4)/C(=N\Nc4nccs4)C3=O)n2)cc1 ; CC(C)(C)OC(=O)CCCCCCCCN ; CC(C)(C)OC(=O)CCCCCCCCNC(=O)c1ccc(-c2csc(N3N=C(c4ccccc4)/C(=N\Nc4nccs4)C3=O)n2)cc1 ; O=C(O)CCCCCCCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCCCCCCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 216-3

Detailed Synthetic Procedure: To a solution of intermediate 216-1 (100 mg, 210.74 umol, 1 eq) and intermediate 216-2 (62.84 mg, 273.96 umol, 1.3 eq) in DMF (1 mL) was added EDCI (121.20 mg, 632.23 umol, 3 eq), NMM (106.58 mg, 1.05 mmol, 115.85 uL, 5 eq) and HOAt (28.68 mg, 210.74 umol, 29.48 uL, 1 eq). The mixture was stirred at 25° C. for 1 hr. LCMS showed SM was consumed completely and 64% of desired mass was detected. The reaction mixture was diluted with water (15 mL) and extracted with DCM (20 mL*3). The combined organic layer was dried over Na₂SO₄, filtered and concentrated in vacuo to give a residue. The residue was purified by column chromatography (SiO₂, PE:EA=1:1 to DCM:MeOH=10:1), then the organic liquid was concentrated in vacuo to give intermediate 216-3 (150 mg, 201.21 umol, 95.48% yield, 92% purity) as a orange solid which was confirmed by LCMS and HNMR.

### Mass Found

Retention time=0.620 min, (M+H)=686.5

Retention time=0.624 min, (M+H)+23=708.6

### NMR Data

¹H NMR (400 MHz, DMSO-d6) δ=8.48-8.46 (m, 1H), 8.21 (br d, J=6.8 Hz, 1H), 8.07 (d, J=8.4 Hz, 1H), 7.95-7.90 (m, 3H), 7.82 (s, 1H), 7.64 (br d, J=3.6 Hz, 1H), 7.54-7.46 (m, 2H), 7.45-7.40 (m, 1H), 7.24 (d, J=3.6 Hz, 1H), 3.26 (br d, J=6.4 Hz, 2H), 2.33-2.32 (m, 2H), 1.55-1.46 (m, 4H), 1.39 (s, 9H), 1.33-1.24 (m, 9H).

### Step 2: Synthesis of Intermediate 216-4

Detailed Synthetic Procedure: To a solution of intermediate 216-3 (100 mg, 145.80 umol, 1 eq) in DCM (0.5 mL) was added HCl/dioxane (4 M, 36.45 uL, 1 eq). The mixture was stirred at 25° C. for 1 hr. LCMS (EC5839-49-P1A1) showed SM was consumed completely and 89% of desired mass was detected. The reaction mixture was concentrated in vacuo to give intermediate 216-4 (100 mg, 141.33 umol, 96.93% yield, 89% purity) as a orange solid.

### Mass:

Retention time=0.532 min, (M+H)=630.3

### Step 3: Synthesis of Compound 137

Detailed Synthetic Procedure: To a solution of intermediate 216-4 (100 mg, 158.79 umol, 1 eq) and intermediate 216-5 (81.72 mg, 158.79 umol, 1 eq) in DMF (2 mL) was added EDCI (91.32 mg, 476.38 umol, 3 eq), NMM (80.31 mg, 793.96 umol, 87.29 uL, 5 eq) and HOAt (21.61 mg, 158.79 umol, 22.21 uL, 1 eq). The mixture was stirred at 25° C. for 1 hr. LCMS showed SM was consumed completely and 88% of desired mass was detected. The mixture was diluted with MeOH (2 ml) and purified by prep-HPLC directly (column: Phenomenex luna C18 150*25 mm*10 um;mobile phase: [water(TFA)-ACN];B %: 48%-78%, 10 min) to give a residue. Then residue was concentrated in vacuo and lyophilized to give Compound 137 (39.79 mg, 35.33 umol, 22.25% yield, 100% purity) as a orange solid which was confirmed by LCMS, SFC and HNMR.

### Mass:

Retention time=0.561 min, (M+H)=1126.4

Retention time=0.558 min, (M+H)=1126.3

### NMR Data:

¹H NMR (400 MHz, DMSO-d6) δ=8.49-8.48 (m, 1H), 8.38-8.37 (m, 1H), 8.16 (br d, J=6.8 Hz, 2H), 8.06 (d, J=8.4 Hz, 2H), 7.97-7.94 (m, 2H), 7.91 (d, J=8.4 Hz, 2H), 7.72 (d, J=4.0 Hz, 1H), 7.65 (d, J=8.4 Hz, 2H), 7.57-7.52 (m, 3H), 7.42 (d, J=8.4 Hz, 2H), 7.35 (d, J=4.0 Hz, 1H), 7.26-7.22 (m, 4H), 7.17-7.13 (m, 1H), 4.34 (br d, J=4.8 Hz, 2H), 4.08 (s, 3H), 4.01-3.95 (m, 2H), 3.89 (br d, J=5.6 Hz, 2H), 3.69-3.58 (m, 2H), 3.28-3.24 (m, 2H), 3.20-3.12 (m, 2H), 2.90-2.83 (m, 1H), 2.56 (br d, J=7.6 Hz, 2H), 2.16-2.14 (m, J=7.4 Hz, 2H), 1.60-1.44 (m, 6H), 1.30 (br s, 10H), 1.19 (d, J=7.2 Hz, 3H).

### SFC Data:

Retention time: 1.030 min, AS-3-IPA+ACN(DEA)-60-3 mL-35T

### Example 217. Synthesis of N-[2-[3-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-3-oxo-propoxy]ethyl]-4-[2-[(4E)-5-oxo-3-phenyl-4-(thiazol-2-ylhydrazono)pyrazol-1-yl]thiazol-4-yl]benzamide (Compound 138)

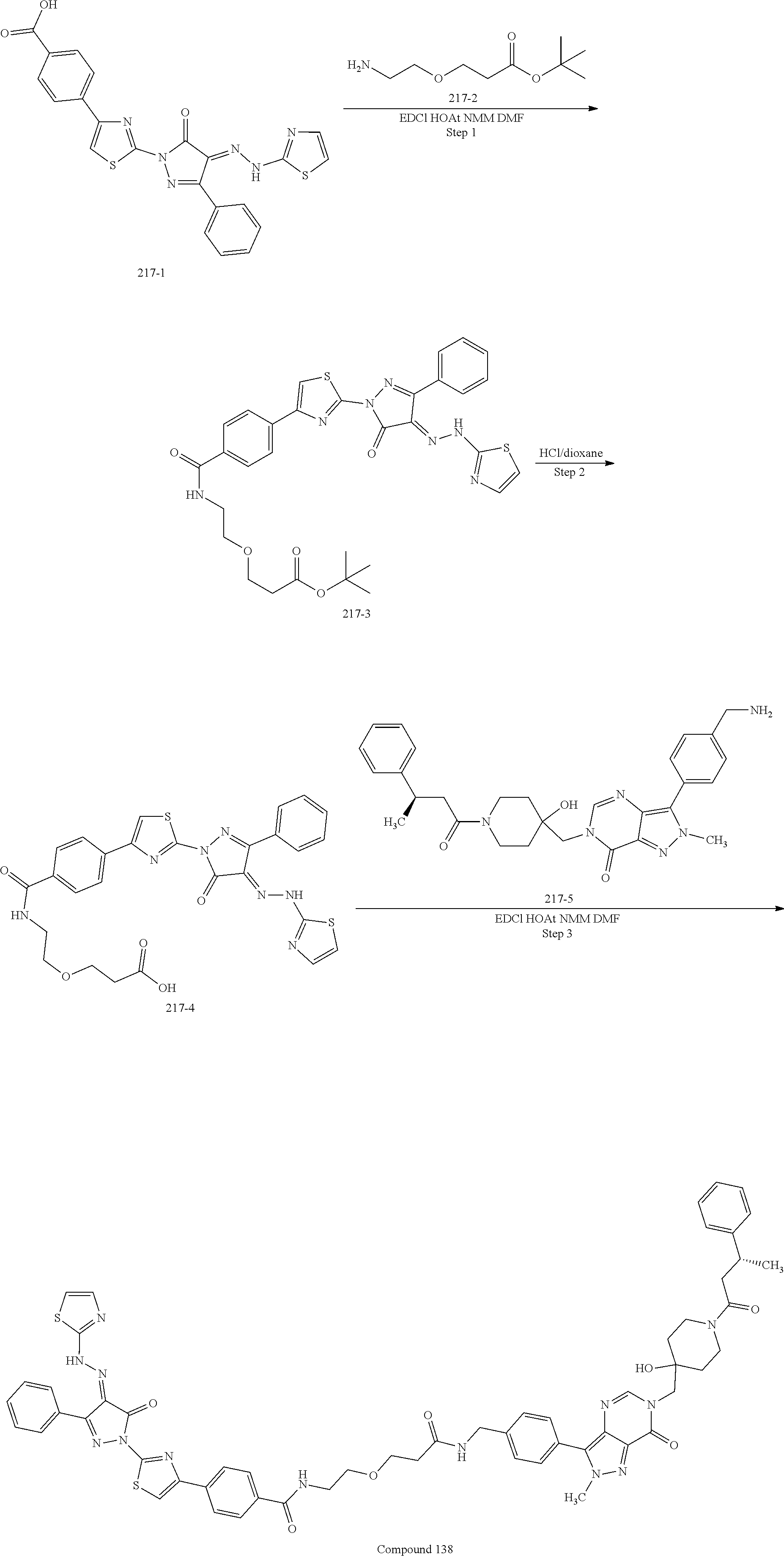 [SMILES: O=C(O)c1ccc(-c2csc(N3N=C(c4ccccc4)/C(=N\Nc4nccs4)C3=O)n2)cc1 ; CC(C)(C)OC(=O)CCOCCN ; CC(C)(C)OC(=O)CCOCCNC(=O)c1ccc(-c2csc(N3N=C(c4ccccc4)/C(=N\Nc4nccs4)C3=O)n2)cc1 ; O=C(O)CCOCCNC(=O)c1ccc(-c2csc(N3N=C(c4ccccc4)/C(=N\Nc4nccs4)C3=O)n2)cc1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCOCCNC(=O)c5ccc(-c6csc(N7N=C(c8ccccc8)/C(=N\Nc8nccs8)C7=O)n6)cc5)cc4)n(C)nc3c2=O)CC1)c1ccccc1]

### Step 1: Synthesis of Intermediate 217-3

To a solution of intermediate 217-1 (80 mg, 168.59 umol, 1 eq) and intermediate 217-2 (47.86 mg, 252.89 umol, 1.5 eq) in DMF (0.8 mL) was added NN (85.26 mg, 842.97 umol, 92.68 uL, 5 eq), EDCI (161.60 mg, 842.97 umol, 5 eq) and HOAt (34.42 mg, 252.89 umol, 35.38 uL, 1.5 eq). The mixture was stirred at 25° C. for 2 hrs. LCMS showed 61.63% of desired compound was detected. TLC (DCM:MeOH=20:1, by UV=254 nm) showed one new main spot (Rt=0.40) was formed. The reaction mixture was washed with water (12 mL) and extracted with EA (20 mL*3), the combined organic phase was dried by Na2SO4, concentrated under reduced pressure to give a residue. The residue was purified by flash silica gel chromatography (40 g Silica Flash Column, Eluent of 0-50% Methanol/Dichloromethane @ 20 mL/min) and the eluent was concentrated to give intermediate 217-3 (92 mg, 110.32 umol, 65.44% yield, 77.43% purity) as a white solid which was confirmed by LCMS.

### Mass Found

LCMS: Retention time=0.554 min, M+H=646.5

LCMS: Retention time=0.553 min, M+H=646.6

### Step 2: Synthesis of Intermediate 217-4

Detailed Synthetic Procedure: To a solution of intermediate 217-3 (92 mg, 110.32 umol, 77.435% purity, 1 eq) in HCl/dioxane (1 mL, 4M), the mixture was stirred at 25° C. for 2 hrs. LC-MS showed 66.18% of desired compound was detected. Concentrated under reduced pressure to give a residue.

The crude product was used into the next step without further purification. Intermediate 217-4 (93 mg, 104.38 umol, 94.62% yield, 66.18% purity) was obtained as a red solid.

### Mass Found

LCMS: Retention time: 0.473 min, (M+H)=590.5

### Step 3: Synthesis of Compound 138

Detailed Synthetic Procedure: To a solution of intermediate 217-4 (20.00 mg, 33.92 umol, 1 eq) and intermediate 217-5 (26.18 mg, 50.88 umol, 1.5 eq) in DMF (0.3 mL) was added NMM (17.15 mg, 169.60 umol, 18.65 uL, 5 eq), HOAt (6.93 mg, 50.88 umol, 7.12 uL, 1.5 eq) and EDCI (32.51 mg, 169.60 umol, 5 eq). The mixture was stirred at 25° C. for 2 hrs. LCMS showed 41.29% of desired compound was detected. The reaction mixture was diluted with water and purified by prep-HPLC (column: Phenomenex luna C18 150*25 mm*10 um; mobile phase: [water(TFA)-ACN];B %: 38%-68%, 10 min), the eluent was concentrated to remove ACN and lyophilized to give Compound 138 (10 mg, 8.79 umol, 25.90% yield, 95.44% purity) as a white solid and confirmed by HNMR, SFC and LCMS.

### Mass Found:

LCMS: Retention time=0.508 min, M+H=1086.2

LCMS: Retention time=0.515 min, M+H=1086.3

SFC: Retention time: AS-3-MeOH+CAN (DEA)-50-3 mL-35T

### NMR Data:

¹HNMR (400 MHz, DMSO-d6) δ=8.54 (t, J=5.2 Hz, 1H), 8.49 (t, J=5.6 Hz, 1H), 8.16 (d, J=6.4 Hz, 2H), 8.06 (d, J=8.4 Hz, 2H), 7.98-7.91 (m, 4H), 7.73 (d, J=4.0 Hz, 1H), 7.65 (d, J=8.0 Hz, 2H), 7.60-7.51 (m, 3H), 7.45 (d, J=8.4 Hz, 2H), 7.37 (d, J=4.0 Hz, 1H), 7.30-7.23 (m, 4H), 7.21-7.15 (m, 1H), 4.38 (d, J=4.8 Hz, 2H), 4.07 (s, 3H), 4.02-3.94 (m, 2H), 3.89-3.87 (m, 1H), 3.72 (t, J=6.4 Hz, 2H), 3.68-3.60 (m, 1H), 3.60-3.54 (m, 2H), 3.51-3.43 (m, 2H), 3.26-3.12 (m, 2H), 2.94-2.81 (m, 1H), 2.65-2.53 (m, 2H), 2.48-2.45 (m, 2H), 1.41-1.23 (m, 4H), 1.22-1.19 (m, 3H).

### Example 218. Synthesis of [N-[7-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-7-oxo-heptyl]-2-[(2E)-2-[5-oxo-3-phenyl-1-(4-phenylthiazol-2-yl)pyrazol-4-ylidene]hydrazino]thiazole-5-carboxamide] (Compound 139)

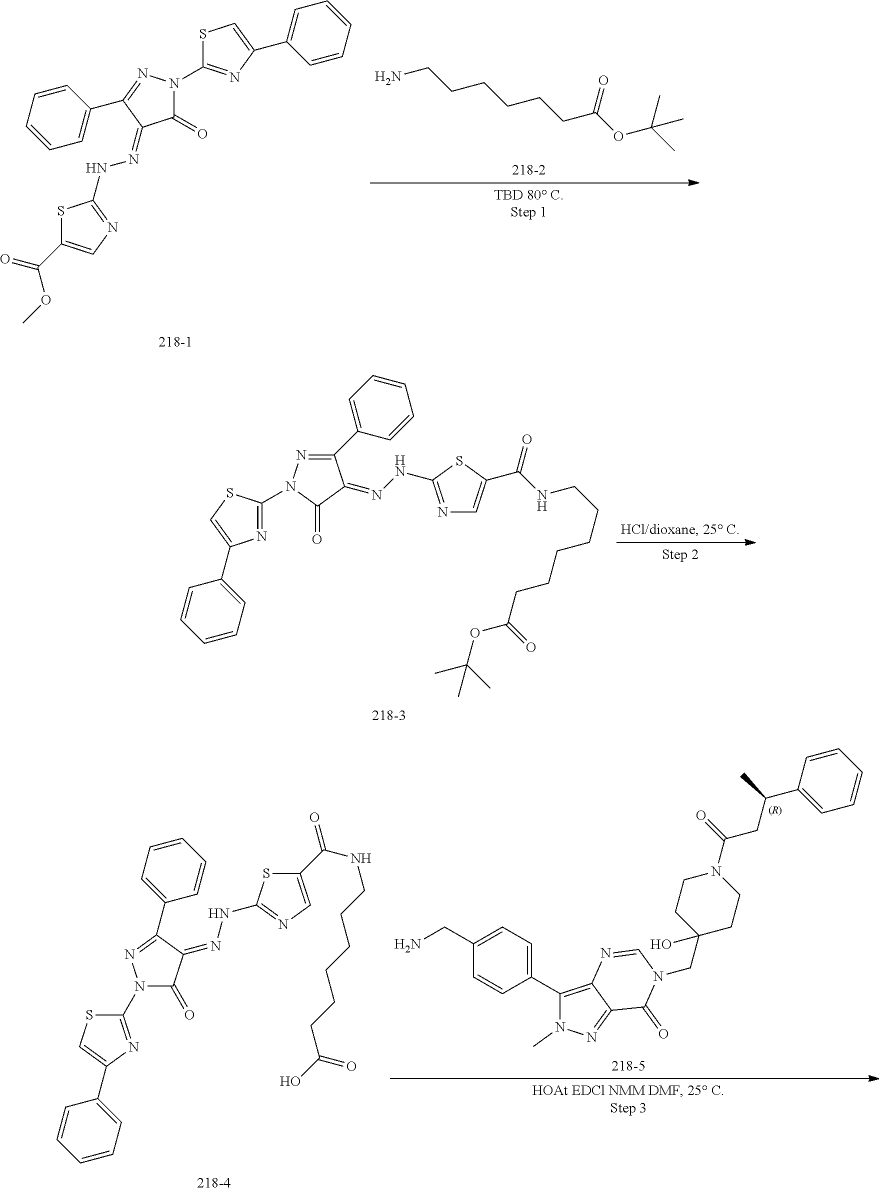

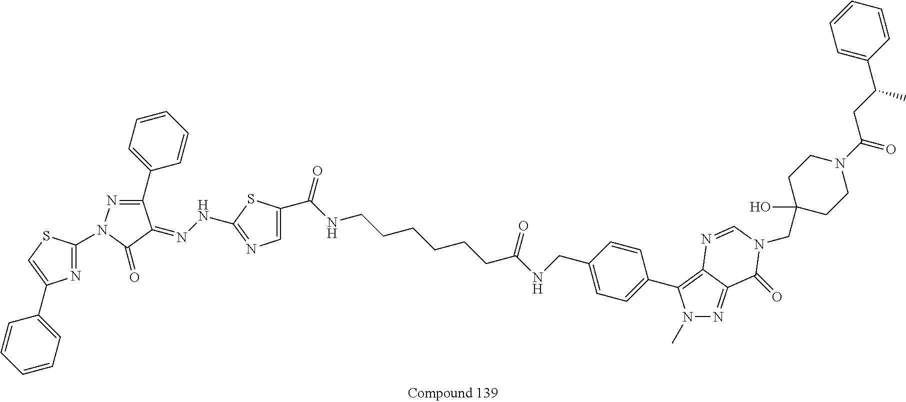 [SMILES: C[C@H](CC(=O)N1CCC(O)(Cn2cnc3c(-c4ccc(CNC(=O)CCCCCCNC(=O)c5cnc(N/N=C6/C(=O)N(c7nc(-c8ccccc8)cs7)N=C6c6ccccc6)s5)cc4)n(C)nc3c2=O)CC1)c1ccccc1]

### Step 1: Synthesis of Intermediate 218-3

To a solution of intermediate 218-1 (100 mg, 204.69 umol, 1 eq) and intermediate 218-2 (49.45 mg, 245.63 umol, 1.2 eq) in THE (1 mL) was added TBD (37.04 mg, 266.10 umol, 1.3 eq). The mixture was stirred at 80° C. for 2 hrs. LCMS showed desired molecular weight was detected. The reaction solution was concentrated in vacuum. The residue was diluted with H₂O (20 mL) and extracted with DCM (25 mL*2). The combined organic layers were washed with brine (10 mL), dried over anhydrous Na₂SO₄, filtered and concentrated in vacuum to get intermediate 218-3 (130 mg, 197.63 umol, 96.55% yield) was obtained as a red oil.

### Mass Found

Retention time: 1.038 min, (M+H)=658.2,

### Step 2: Synthesis of Intermediate 218-4

Detailed Synthetic Procedure: To a solution of intermediate 218-3 (120 mg, 182.43 umol, 1 eq) in HCl/dioxane (1 mL). The mixture was stirred at 25° C. for 1 hr. LCMS (EW33821-51-P1B) showed desired molecular weight was detected. The reaction solution was concentrated in vacuum to get intermediate 218-4 (116 mg, 181.77 umol, 99.64% yield, HCl) was obtained as a red solid.

### Mass Found

Retention time: 1.002 min, (M+H)=602.4

### Step 3: Synthesis of Compound 139

Detailed Synthetic Procedure: To a solution of intermediate 218-4 (116 mg, 192.79 umol, 1 eq) and intermediate 218-5 (118.99 mg, 215.92 umol, 1.12 eq, HCl) in DMF (1 mL) was added EDCI (110.87 mg, 578.36 umol, 3 eq) and HOAt (26.24 mg, 192.79 umol, 26.97 uL, 1 eq) and NN (97.50 mg, 963.94 umol, 105.98 uL, 5 eq). The mixture was stirred at 25° C. for 2 hrs. LCMS showed desired molecular weight was detected. The residue was diluted with H₂O (20 mL) and extracted with DCM (25 mL*2). The combined organic layers were washed with brine (10 mL), dried with anhydrous Na₂SO₄, filtered and concentrated in vacuum. The crude was purified by prep-HPLC (column: Waters Xbridge 150*25 mm*5 um; mobile phase: [water (ammonia hydroxide v/v)-ACN]; B %: 26%-56%, 8.5 min) and the eluent was lyophilized to get Compound 139 (18.49 mg, 16.41 umol, 8.51% yield, 97.48% purity) was obtained as orange solid, which was confirmed by 1HNMR, LCMS and SFC.

### Mass:

Retention time: 1.035 min, (M/2+H)=550.2

Retention time: 0.800 min, (M+H)=1098.5

### NMR Data:

1H NMR (400 MHz, DMSO-d6) δ=8.46-8.40 (m, 1H), 8.23-8.14 (m, 3H), 8.05-7.96 (m, 3H), 7.77 (s, 1H), 7.67 (m, 2H), 7.58-7.50 (m, 3H), 7.49-7.43 (m, 4H), 7.36 (m, 1H), 7.29-7.24 (m, 4H), 7.20-7.12 (m, 1H), 4.36 (m, 2H), 4.10 (s, 3H), 4.06-3.96 (m, 2H), 3.91 (m, 1H), 3.70-3.61 (m, 2H), 3.27-3.17 (m, 4H), 2.94-2.75 (m, 3H), 2.19 (s, 2H), 1.64-1.43 (m, 6H), 1.33 (m, 8H), 1.21 (m, 3H).

SFC: Rt=4.313 min; method details: column: Chiralcel OD-RH 150×4.6 mm I.D., Sum; mobile phase: A (water with 0.375% TFA); B (acetonitril with 0.1875% TFA); B in A from 10% to 80%; flow rate: 1.0 mL/min; wavelength: 220 nm

### Example 219. Synthesis of [(R)-3-(((9-ethyl-7-(4-methylthiophen-2-yl)-9H-carbazol-3-yl)methyl)amino)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)propanamide](Compound 140)

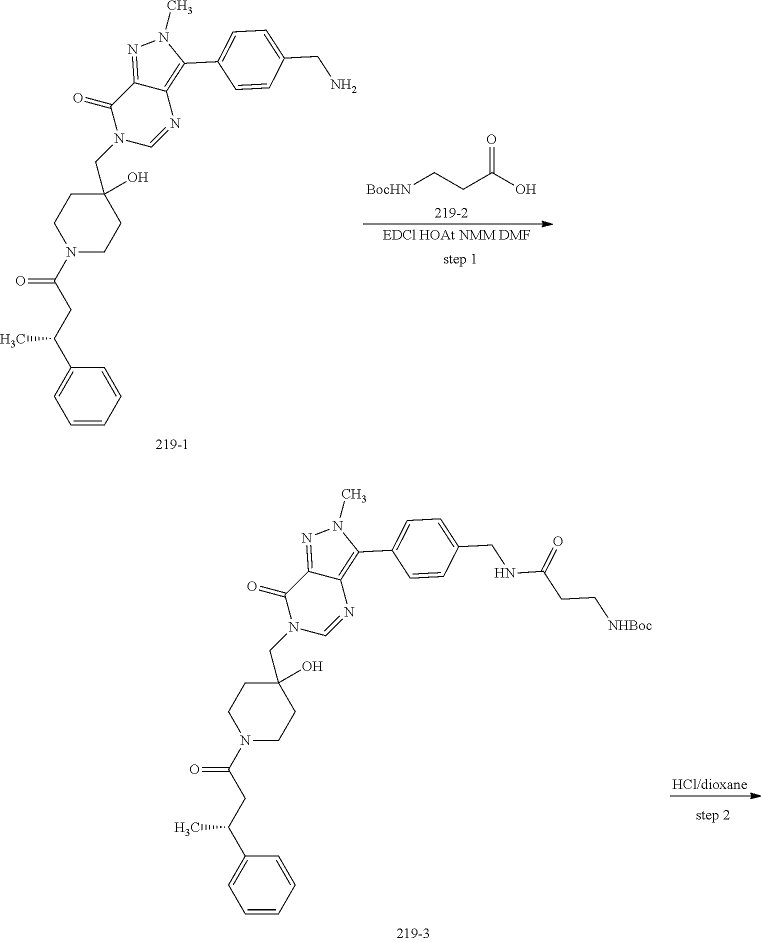 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCC(=O)O ; C[C@H](CC(=O)N1CCC(O)(Cn2cnc3c(-c4ccc(CNC(=O)CCNC(=O)OC(C)(C)C)cc4)n(C)nc3c2=O)CC1)c1ccccc1]

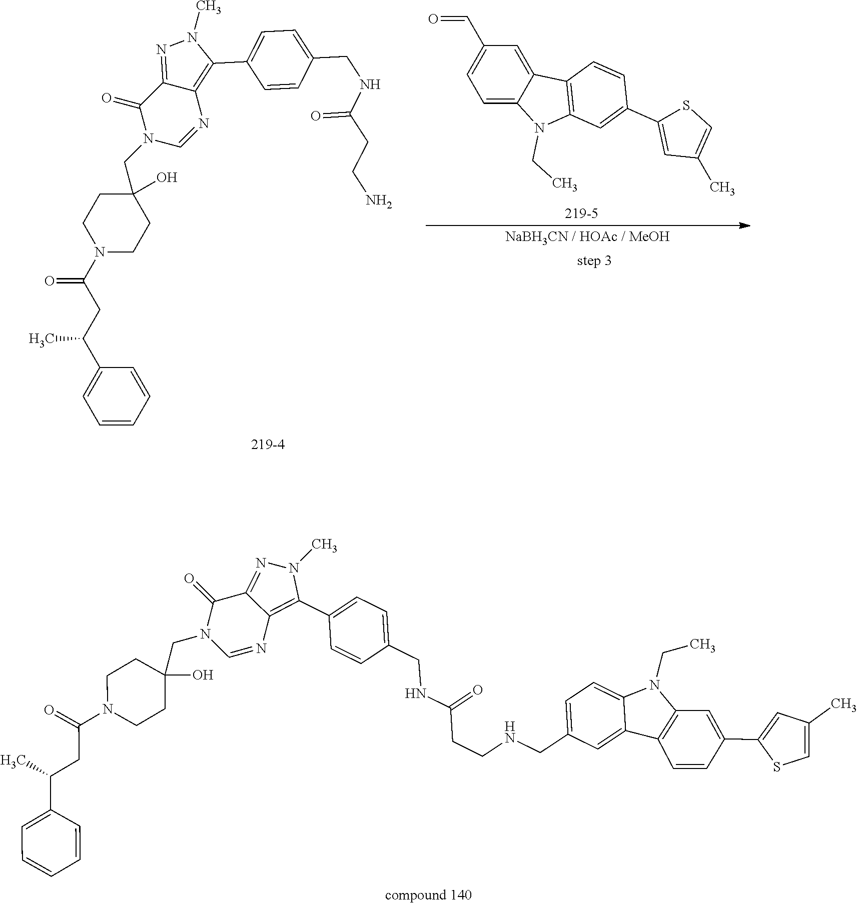 [SMILES: C[C@H](CC(=O)N1CCC(O)(Cn2cnc3c(-c4ccc(CNC(=O)CCN)cc4)n(C)nc3c2=O)CC1)c1ccccc1 ; CCn1c2ccc(C=O)cc2c2ccc(-c3cc(C)cs3)cc21 ; CCn1c2ccc(CNCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 219-3

To a solution of intermediate 219-1 (70 mg, 127.02 umol, 1 eq, HCl) in DMF (0.7 mL) was added NMM (64.24 mg, 635.12 umol, 69.83 uL, 5 eq), intermediate 219-2 (36.05 mg, 190.54 umol, 1.5 eq), HOAt (51.87 mg, 381.07 umol, 53.31 uL, 3 eq) and EDCI (121.75 mg, 635.12 umol, 5 eq). The mixture was stirred at 25° C. for 2 hrs. LCMS showed a main peak with desired mass was detected. The mixture was washed with H₂O (5 ml), and extracted with EA 18 ml (3*8 mL). The organic phase was concentrated under reduced pressure to give a white solid, which was purified by column chromatography (SiO₂, MeOH/DCM=0%˜20%) and concentrated to get intermediate 219-3 (90 mg, crude) was obtained as a white solid which was confirmed by LCMS.

### Mass Found

Retention time: 0.428 min, (M+H)=686.5

Retention time: 0.432 min, (M+H)=686.4

### Step 2: Synthesis of Intermediate 219-4

Detailed Synthetic Procedure: To a solution of intermediate 219-3 (90 mg, 131.23 umol, 1 eq) in HCl/dioxane (1 mL). The mixture was stirred at 25° C. for 1 hr. LCMS showed a major peak with mass was detected. The reaction mixture was concentrated under reduced pressure to get intermediate 219-4 (70 mg, 112.51 umol, 85.74% yield, HCl) was obtained as a white solid which.

### Mass Found

Retention time: 0.345 min, (M+H)=586.4

### Step 3: Synthesis of compound 140

Detailed Synthetic Procedure: To a solution of intermediate 219-4 (70 mg, 119.52 umol, 1 eq) in MeOH (1 mL) was added TEA (48.37 mg, 478.06 umol, 66.54 uL, 4 eq), then the mixture was stirred at 25° C. for 15 min. After that the mixture was added NaBH3CN (45.06 mg, 717.10 umol, 6 eq), intermediate 219-5 (30.54 mg, 95.61 umol, 0.8 eq) and AcOH (43.06 mg, 717.10 umol, 41.01 uL, 6 eq). The mixture was stirred at 25° C. for 3 hrs. LCMS showed a major peak with mass was detected. The reaction mixture was filtered and the filter liquor was a crude product, which was purified by prep-HPLC (column: Phenomenex luna C18 150*25 mm*10 um; mobile phase: [water (FA)-ACN];B %: 30%-60%, 10 min). The eluent was concentrated and lyophilized to get compound 140 (45 mg, 50.61 umol, 42.35% yield, 100% purity) was obtained as a white solid which was confirmed by HNMR, SFC and LCMS.

### Mass Found

Retention time: 0.585 min, (M+H)=890.1

Retention time: 0.584 min, (M+H)=890.3

### NMR Data

¹H NMR (400 MHz, DMSO-d6) δ=8.64 (t, J=5.2 Hz, 1H), 8.18-8.13 (m, 1H), 8.08 (d, J=8.4 Hz, 1H), 7.95 (d, J=10.0 Hz, 1H), 7.84 (s, 1H), 7.66-7.57 (m, 3H), 7.52-7.40 (m, 5H), 7.30-7.21 (m, 4H), 7.18-7.10 (m, 2H), 4.85 (s, 1H), 4.54-4.43 (m, 2H), 4.38 (d, J=5.6 Hz, 2H), 4.12-3.84 (m, 9H), 3.67-3.60 (m, 1H), 3.21-3.14 (m, 3H), 3.01 (s, 2H), 2.93-2.84 (m, 1H), 2.63-2.54 (m, 2H), 2.28 (s, 3H), 1.35-1.26 (m, 5H), 1.23-1.12 (m, 5H).

### SFC Data

SFC: Retention time: OD-3-MeOH+ACN (DEA)-60-3 mL-35T

### Example 220. Synthesis of [(R)-5-(((9-ethyl-7-(4-methylthiophen-2-yl)-9H-carbazol-3-yl)methyl)amino)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)pentanamide](Compound 141)

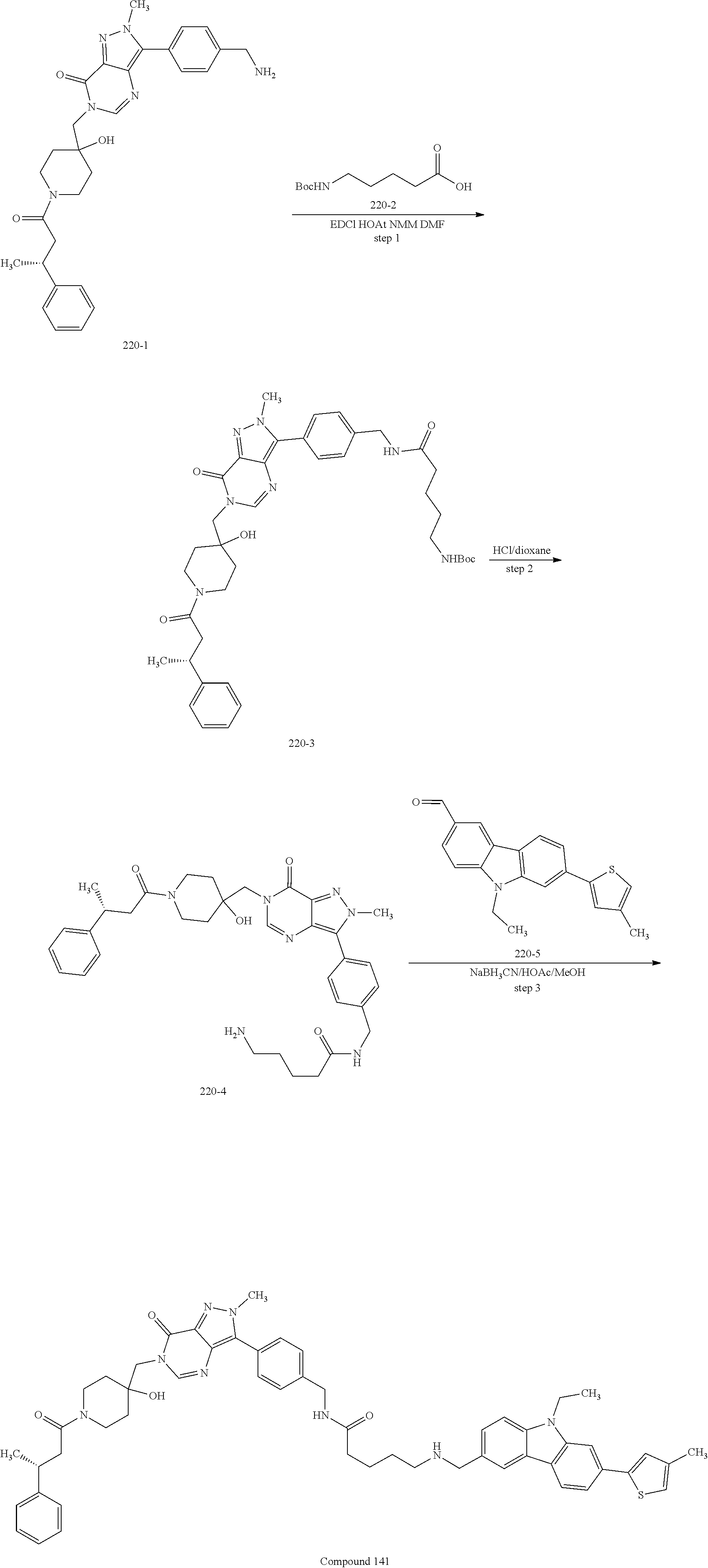 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCCCC(=O)O ; C[C@H](CC(=O)N1CCC(O)(Cn2cnc3c(-c4ccc(CNC(=O)CCCCNC(=O)OC(C)(C)C)cc4)n(C)nc3c2=O)CC1)c1ccccc1 ; C[C@H](CC(=O)N1CCC(O)(Cn2cnc3c(-c4ccc(CNC(=O)CCCCN)cc4)n(C)nc3c2=O)CC1)c1ccccc1 ; CCn1c2ccc(C=O)cc2c2ccc(-c3cc(C)cs3)cc21 ; CCn1c2ccc(CNCCCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 220-3

Detailed Synthetic Procedure: To a solution of intermediate 220-1 (70 mg, 127.02 umol, 1 eq, HCl) in DMF (0.7 mL) was added NMM (64.24 mg, 635.12 umol, 69.83 uL, 5 eq) intermediate 220-2 (41.40 mg, 190.54 umol, 1.5 eq), HOAt (51.87 mg, 381.07 umol, 53.31 uL, 3 eq) and EDCI (121.75 mg, 635.12 umol, 5 eq). The mixture was stirred at 25° C. for 4 hrs. LCMS showed a main peak with desired mass was detected. The mixture was washed with H₂O (5 ml), and extracted with EA 18 ml (3×8 mL). The organic phase was concentrated under reduced pressure to give a white solid, which was purified by column chromatography (SiO₂, MeOH/DCM=0%˜20%) to get intermediate 220-3 (100 mg, crude) was obtained as a white solid which was confirmed by LCMS.

### Mass Found

Retention time: 0.438 min, (M+H)=714.3

Retention time: 0.450 min, (M+H)=714.5

### Step 2: Synthesis of Intermediate 220-4

Detailed Synthetic Procedure: To a solution of intermediate 220-3 (100 mg, 140.08 umol, 1 eq) in HCl/dioxane (1 mL, 4M). The mixture was stirred at 25° C. for 1 hr. LCMS showed a major peak with mass was detected. The reaction mixture was concentrated under reduced pressure to get intermediate 220-4 (80 mg, 123.04 umol, 87.83% yield, HCl) was obtained as a white solid.

### Mass Found

Retention time: 0.343 min, (M+H)=614.4

### Step 3: Synthesis of Compound 141

Detailed Synthetic Procedure: To a solution of intermediate 220-4 (80.00 mg, 130.35 umol, 1 eq) in MeOH (1 mL) was added TEA (52.76 mg, 521.39 umol, 72.57 uL, 4 eq), then the mixture was stirred at 25° C. for 15 min. After that the mixture was added NaBH3CN (49.15 mg, 782.08 umol, 6 eq), intermediate 220-5 (33.31 mg, 104.28 umol, 0.8 eq) and AcOH (46.97 mg, 782.08 umol, 44.73 uL, 6 eq). The mixture was stirred at 25° C. for 12 hrs. LCMS showed desired mass was detected. The reaction mixture was filtered and the filter liquor was a crude product, which was purified by prep-HPLC (column: Phenomenex luna C18 150*25 mm*10 um;mobile phase: [water(FA)-ACN];B %: 29%-59%, 10 min). The eluent was concentrated and lyophilized to get Compound 141 (26 mg, 26.84 umol, 20.59% yield, 94.69% purity) was obtained as a white solid and characterized by HNMR and LCMS.

### Mass Found

Retention time: 0.480 min, (M+H)=917.3

Retention time: 0.484 min, (M+H)=917.3

### NMR Data:

¹HNMR (400 MHz, METHANOL-d4) δ=8.46 (s, 1H), 8.10 (s, 1H), 7.98 (d, J=7.6 Hz, 1H), 7.84-7.74 (m, 1H), 7.67 (s, 1H), 7.49-7.41 (m, 6H), 7.34-7.15 (m, 6H), 6.95 (s, 1H), 4.46-4.34 (m, 4H), 4.29-3.49 (m, 10H), 3.28-2.81 (m, 6H), 2.79-2.32 (m, 5H), 1.82-1.72 (m, 4H), 1.42-1.18 (m, 10H)

### SFC Data

SFC: Retention time: OD-3-MeOH+ACN(DEA)-60-3 mL-35T

### Example 221. Synthesis of [(R)-7-(((9-ethyl-7-(4-methylthiophen-2-yl)-9H-carbazol-3-yl)methyl)amino)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)heptanamide](Compound 142)

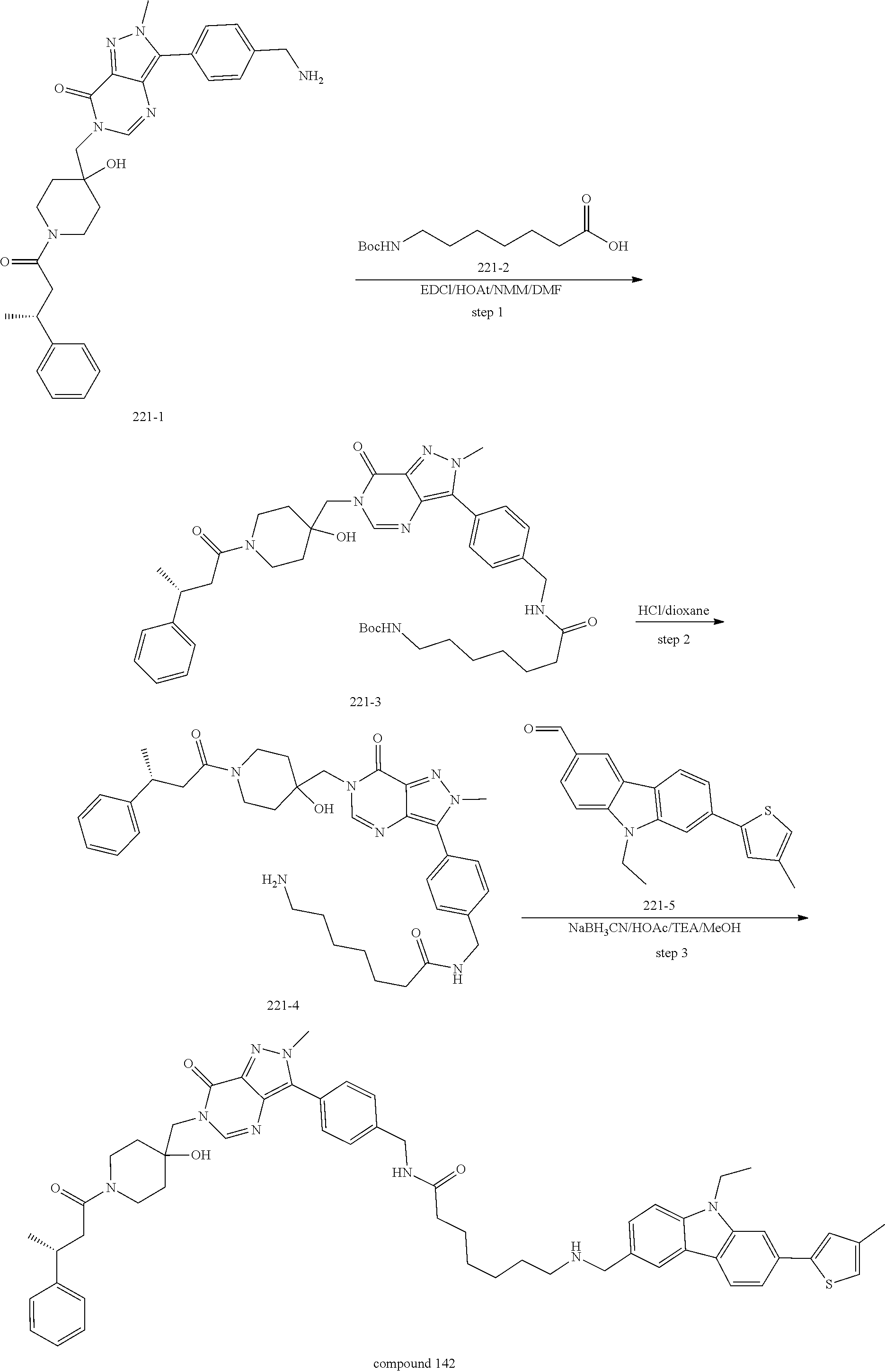 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCCCCCC(=O)O ; C[C@H](CC(=O)N1CCC(O)(Cn2cnc3c(-c4ccc(CNC(=O)CCCCCCNC(=O)OC(C)(C)C)cc4)n(C)nc3c2=O)CC1)c1ccccc1 ; C[C@H](CC(=O)N1CCC(O)(Cn2cnc3c(-c4ccc(CNC(=O)CCCCCCN)cc4)n(C)nc3c2=O)CC1)c1ccccc1 ; CCn1c2ccc(C=O)cc2c2ccc(-c3cc(C)cs3)cc21 ; CCn1c2ccc(CNCCCCCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 221-3

Detailed Synthetic Procedure: To a solution of intermediate 221-2 (10.68 mg, 43.55 umol, 1.2 eq) in DMF (0.2 mL) was added EDCI (13.91 mg, 72.58 umol, 2 eq), HOAt (4.94 mg, 36.29 umol, 5.08 uL, 1 eq) and NMM (18.35 mg, 181.46 umol, 19.95 uL, 5 eq). The mixture was stirred at 25° C. for 30 min. Then intermediate 221-1 (20 mg, 36.29 umol, 1 eq, HCl) was added into the mixture, the mixture was stirred at 25° C. for 1 hr. LCMS showed 77.59% of desired mass was detected. TLC (EA/MeOH=10:1, Rt=0.5) showed a new spot was detected. The reaction mixture was added H2O (1 mL) and then extracted with EA (1 mL*3), dried by Na2SO4, filtered and concentrated to give crude product. The residue was purified by column chromatography (SiO2, EA/MeOH=1/0 to 0/1), the eluent was concentrated to give intermediate 221-3 (20 mg, 24.89 umol, 68.57% yield, 92.31% purity) as red solid was confirmed by LCMS.

### Mass Data

LCMS: Retention time: 0.466 min, (M+H)=742.6

LCMS: Retention time: 0.468 min, (M+H)=742.7

### Step 2: Synthesis of Intermediate 221-4

Detailed Synthetic Procedure: To a solution of intermediate 221-3 (20 mg, 26.96 umol, 1 eq) in dioxane (0.1 mL) was added HCl/dioxane (4 M, 0.2 mL). The mixture was stirred at 25° C. for 0.5 hr. LCMS (EC3406-254-P1A1) showed 85.202% of desired mass was detected. The reaction mixture was concentrated to give crude product. The residue was used in next step directly and no further purification. The intermediate 221-4 (20 mg, crude, HCl) as red solid was confirmed by LCMS.

### Mass Data

LCMS: Retention time: 0.350 min, (M+H)=642.4

LCMS: Retention time: 0.350 min, (M+H)=642.4

### Step 3: Synthesis of Compound 142

Detailed Synthetic Procedure: To a solution of intermediate 221-4 (10 mg, 14.74 umol, 1 eq, HCl) in MeOH (0.1 mL) was added TEA (4.48 mg, 44.23 umol, 6.16 uL, 3 eq), the mixture was stirred at 25° C. for 0.5 hr, then intermediate 221-5 (4.71 mg, 14.74 umol, 1 eq) and AcOH (5.31 mg, 88.46 umol, 5.06 uL, 6 eq) was added the reaction mixture. The mixture was stirred at 25° C. for 0.5 hr and then NaBH3CN (2.78 mg, 44.23 umol, 3 eq) was added the reaction and stirred at 25° C. for 1 hr. LCMS showed 39.120% of desired mass was detected. The reaction mixture was added H2O (1 mL) and then extracted with EA (1 mL*3), the combined organic phase was dried by Na2SO4, concentrated to give crude product. The residue was purified by prep-HPLC (column: Phenomenex luna C18 150*25 mm*10 um;mobile phase: [water(FA)-ACN];B %: 30%-60%, 10 min) and the eluent was lyophilized to give Compound 1 (10 mg, 10.39 umol, 70.50% yield, 98.25% purity) as white solid was confirmed by LCMS, HNMR, and SFC.

### Mass Data

LCMS: Retention time: 0.626 min, (M+H)=945.0

LCMS: Retention time: 0.493 min, (M+H)=945.4

### NMR Data

¹H NMR (400 MHz, DMSO-d6) δ=8.41-8.34 (m, 1H), 8.28 (s, 1H), 8.09 (s, 2H), 7.96 (br d, J=9.6 Hz, 1H), 7.84 (s, 1H), 7.66 (br d, J=7.6 Hz, 2H), 7.55 (br d, J=8.4 Hz, 1H), 7.49 (s, 1H), 7.47-7.41 (m, 4H), 7.26 (br s, 4H), 7.19-7.14 (m, 1H), 7.13 (s, 1H), 4.88-4.84 (m, 1H), 4.48 (br d, J=6.4 Hz, 2H), 4.35 (br d, J=5.6 Hz, 2H), 4.11-4.06 (m, 3H), 4.05-4.00 (m, 1H), 3.95 (br s, 3H), 3.90 (br d, J=5.6 Hz, 1H), 2.92-2.87 (m, 2H), 2.66-2.62 (m, 2H), 2.28 (br s, 5H), 2.19-2.15 (m, 4H), 1.62-1.45 (m, 6H), 1.36-1.29 (m, 9H), 1.21 (br d, J=6.4 Hz, 3H).

SFC: Retention time: 1.024 min, OD-3-IPA+ACN(DEA)-60-3 mL-35T.

### Example 222. Synthesis of [[9-[[9-ethyl-7-(4-methyl-2-thienyl)carbazol-3-yl]methylamino]-N-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methyl]nonanamide]] (Compound 143)

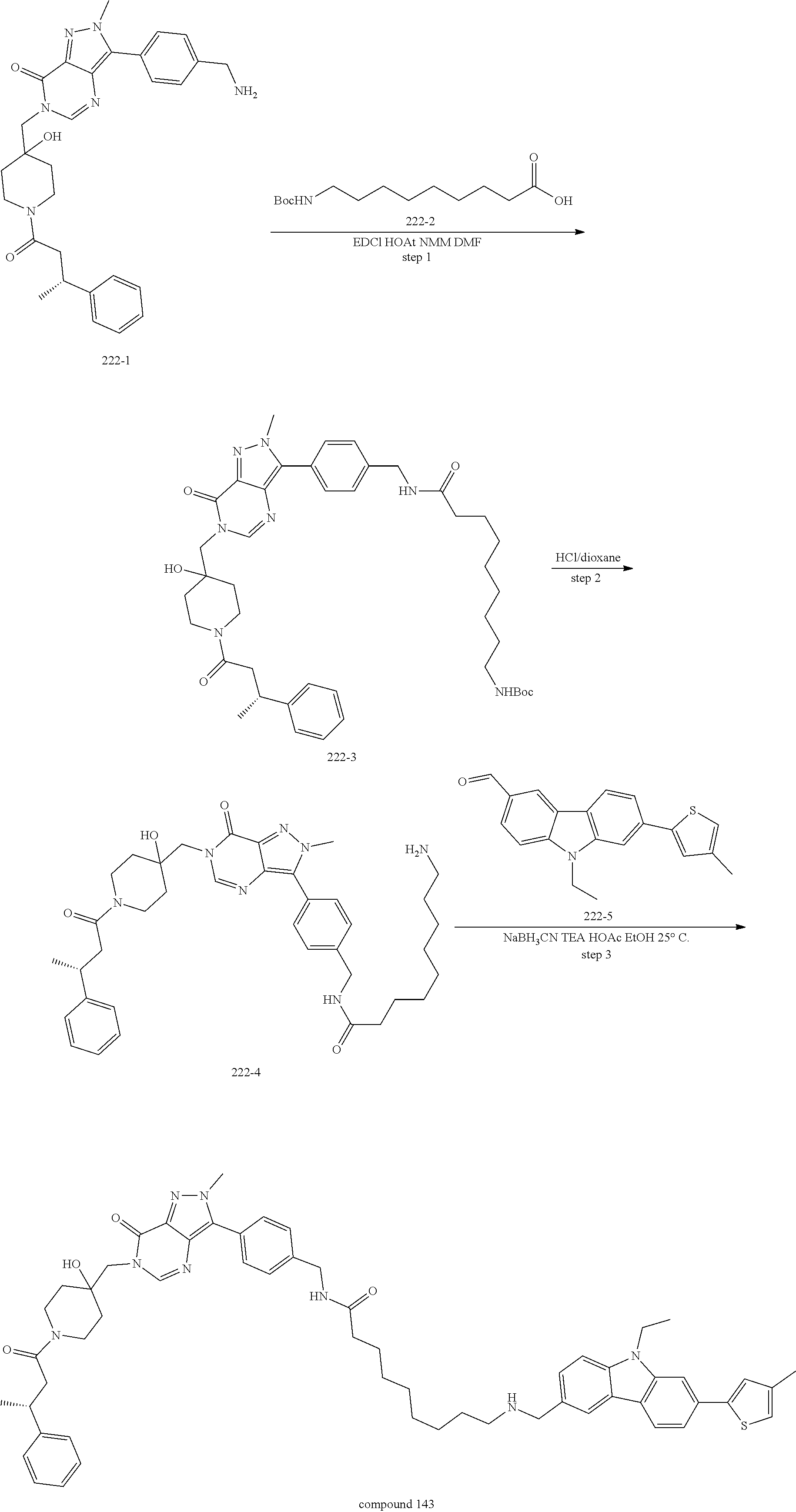 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCCCCCCCC(=O)O ; C[C@H](CC(=O)N1CCC(O)(Cn2cnc3c(-c4ccc(CNC(=O)CCCCCCCCNC(=O)OC(C)(C)C)cc4)n(C)nc3c2=O)CC1)c1ccccc1 ; C[C@H](CC(=O)N1CCC(O)(Cn2cnc3c(-c4ccc(CNC(=O)CCCCCCCCN)cc4)n(C)nc3c2=O)CC1)c1ccccc1 ; CCn1c2ccc(C=O)cc2c2ccc(-c3cc(C)cs3)cc21 ; CCn1c2ccc(CNCCCCCCCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 222-3

Detailed Synthetic Procedure: To a solution of intermediate 222-1 (100 mg, 181.46 umol, 1 eq, HCl) and intermediate 222-2 (74.41 mg, 272.19 umol, 1.5 eq) in DMF (1 mL) was added EDCI (173.93 mg, 907.31 umol, 5 eq), HOAt (24.70 mg, 181.46 umol, 25.38 uL, 1 eq) and NMM (183.54 mg, 1.81 mmol, 199.50 uL, 10 eq). The mixture was stirred at 25° C. for 1 hr. LCMS (EC3201-311-P1A1) showed Reactant 1 was consumed and one major peak with desired mass was detected. The reaction was quenched with H2O (5 mL). The mixture was extract with EA (10 mL*3). The combined organic layers dried over Na2SO4, filtered and concentrated under reduced pressure to give a residue. The residue was purified by flash silica gel chromatography (20 g Silica Flash Column, Eluent of 0˜100% DCM/MeOH @ 60 mL/min, DCM:MeOH=10:1, Rt=0.5) and the eluent was concentrated under reduced pressure to give intermediate 222-3 (100 mg, 120.78 umol, 66.56% yield, 93% purity) as a yellow solid and confirmed by LCMS.

### Mass Data

LCMS: Retention time: 0.442 min, (M+H)=770.4

LCMS: Retention time: 0.500 min, (M+H)=770.3

### Step 2: Synthesis of Intermediate 222-4

Detailed Synthetic Procedure: To a solution of intermediate 222-3 (100 mg, 129.88 umol, 1 eq) in dioxane (1 mL) was added HCl/dioxane (4 M, 1 mL). The mixture was stirred at 25° C. for 0.5 hr. LCMS showed Reactant 1 was consumed and one major peak with desired mass was detected. The mixture was filtered and concentrated under reduced pressure to give a residue. The crude product was used into the next step without further purification. Intermediate 222-4 (100 mg, crude, HCl) was obtained as a white solid.

### Mass Data

LCMS: Retention time: 0.370 min, (M+H)=670.3

### Step 3: Synthesis of Compound 143

Detailed Synthetic Procedure: To a solution of intermediate 222-4 (80 mg, 113.26 umol, 1 eq, HCl) in EtOH (0.8 mL) was added TEA (45.84 mg, 453.06 umol, 63.06 uL, 4 eq) was stirred at 25° C. for 15 min, and then was added AcOH (40.81 mg, 679.58 umol, 38.87 uL, 6 eq) and intermediate 222-5 (36.18 mg, 113.26 umol, 1 eq), the mixture was stirred at 25° C. for 15 min, followed by addition of NaBH3CN (14.24 mg, 226.53 umol, 2 eq). The resulting mixture was stirred at 25° C. for 11.5 hrs. LCMS (EC3201-318-P1A2) showed 18% of Reactant 1 remained and 67% of desired mass was detected. The mixture was filtered and filter liquor was used into purification. The residue was purified by prep-HPLC (column: Phenomenex luna C18 150*25 mm*10 um; mobile phase: [water (FA)-ACN]; B %: 35%-65%, 10 min) and the eluent was lyophilized to give Compound 143 (49 mg, 47.59 umol, 42.02% yield, 99.004% purity, FA) as off-white solid and confirmed by LCMS, SFC, HNMR.

### Mass Data

LCMS: Retention time: 0.500 min, (M+H)=973.6

LCMS: Retention time: 0.509 min, (M+H)=973.9

### NMR Data

¹H NMR (400 MHz, DMSO-d6) δ=8.30-8.26 (m, 1H), 8.20 (s, 1H), 8.05-7.97 (m, 2H), 7.87 (d, J=10.0 Hz, 1H), 7.75 (s, 1H), 7.55 (d, J=7.2 Hz, 2H), 7.48 (d, J=8.4 Hz, 1H), 7.43-7.29 (m, 5H), 7.15 (s, 4H), 7.08-6.99 (m, 2H), 4.42-4.35 (m, 2H), 4.24 (d, J=5.6 Hz, 2H), 3.99 (s, 3H), 3.95-3.85 (m, 4H), 3.80 (d, J=6.8 Hz, 1H), 3.57-3.50 (m, 1H), 3.18-3.02 (m, 3H), 2.84-2.71 (m, 1H), 2.64-2.57 (m, 2H), 2.47 (d, J=7.2 Hz, 1H), 2.18 (s, 3H), 2.08-2.04 (m, 2H), 1.44 (s, 4H), 1.29-1.07 (m, 18H).

### SFC Data

SFC: Retention time: 0.747 min, OD-3-MeOH+CAN (DEA)-60-3 mL-35T

### Example 223. Synthesis of [[4-[[9-ethyl-7-(4-methyl-2-thienyl)carbazol-3-yl]methylamino]-N—[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methyl]butanamide]] (Compound 144)

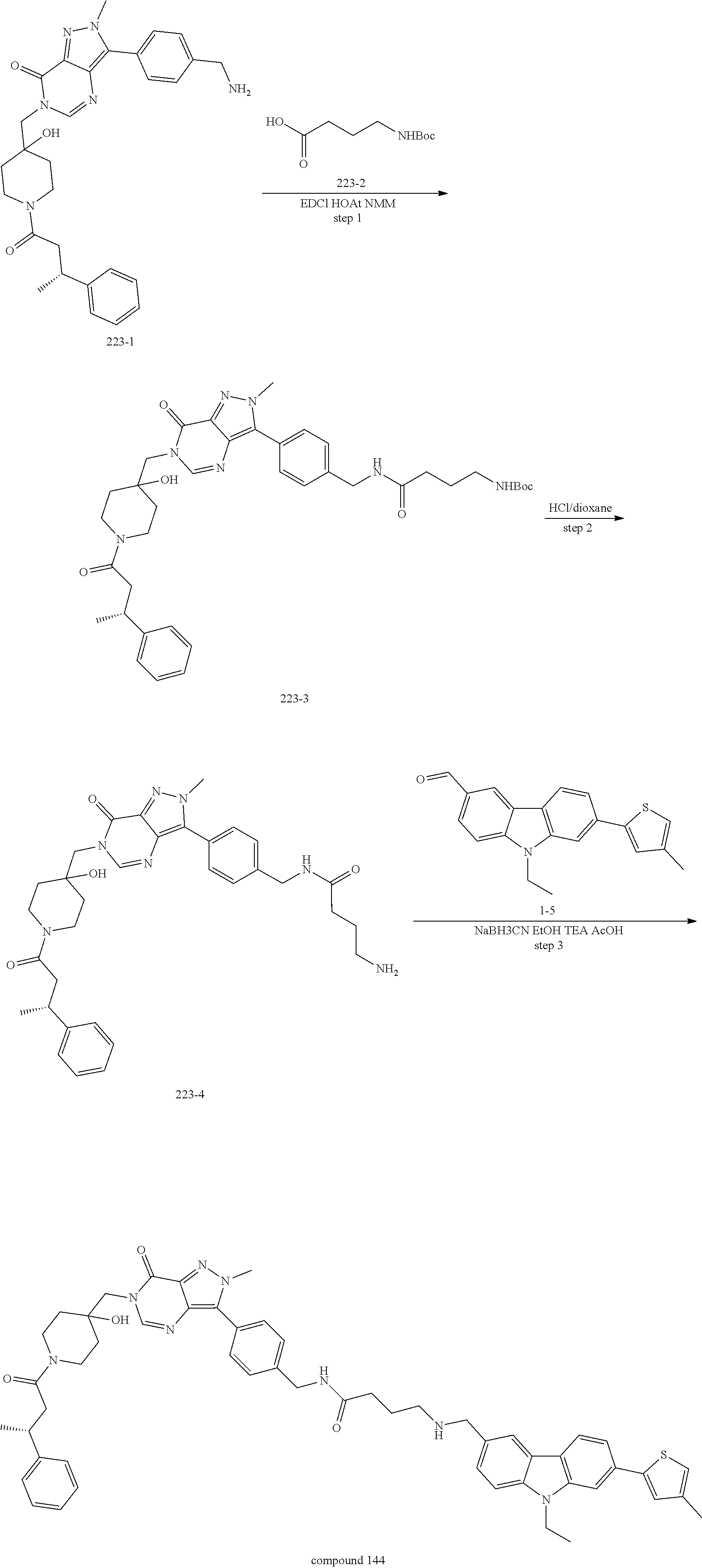 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCCC(=O)O ; C[C@H](CC(=O)N1CCC(O)(Cn2cnc3c(-c4ccc(CNC(=O)CCCNC(=O)OC(C)(C)C)cc4)n(C)nc3c2=O)CC1)c1ccccc1 ; C[C@H](CC(=O)N1CCC(O)(Cn2cnc3c(-c4ccc(CNC(=O)CCCN)cc4)n(C)nc3c2=O)CC1)c1ccccc1 ; CCn1c2ccc(C=O)cc2c2ccc(-c3cc(C)cs3)cc21 ; CCn1c2ccc(CNCCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 223-3

Detailed Synthetic Procedure: To a solution of intermediate 223-1 (100 mg, 181.46 umol, 1 eq, HCl) and intermediate 223-2 (55.32 mg, 272.19 umol, 1.5 eq) in DMF (1 mL) was added EDCI (173.93 mg, 907.31 umol, 5 eq), HOAt (24.70 mg, 181.46 umol, 25.38 uL, 1 eq) and NMM (183.54 mg, 1.81 mmol, 199.50 uL, 10 eq). The mixture was stirred at 25° C. for 1 hr. LCMS showed Reactant 1 was consumed completely and one major peak with desired mass was detected. The reaction was quenched with H2O (5 mL). The mixture was extract with EA (10 mL*3). The combined organic layers dried over Na2SO4, filtered and concentrated under reduced pressure to give a residue. The residue was purified by flash silica gel chromatography (ISCO®; 20 g SepaFlash® Silica Flash Column, Eluent of 0-100% DCM/MeOH @ 60 mL/min, DCM:MeOH=10:1, Rt=0.5) and the eluent was concentrated under reduced pressure to give intermediate 223-3 (100 mg, 130.03 umol, 71.66% yield, 91% purity) as a yellow solid which was confirmed by LCMS

### Mass Data

LCMS: Retention time: 0.497 min, (M+H)=700.5

LCMS: Retention time: 0.279 min, (M+H)=700.1

### Step 2: Synthesis of Intermediate 223-4

Detailed Synthetic Procedure: To a solution of intermediate 223-3 (100 mg, 142.89 umol, 1 eq) in dioxane (1 mL) was added HCl/dioxane (4 M, 1 ml). The mixture was stirred at 25° C. for 0.5 hr. LCMS (EC3201-314-P1A1) showed Reactant 1 was consumed and one major peak with desired mass was detected. The mixture was filtered and concentrated under reduced pressure to give a residue. The crude product was used into the next step without further purification. Intermediate 223-4 (100 mg, crude, HCl) was obtained as a white solid.

### Mass Data

LCMS: Retention time: 0.339 min, (M+H)=600.2

### Step 3: Synthesis of Compound 144

Detailed Synthetic Procedure: To a solution of intermediate 223-4 (80 mg, 125.75 umol, 1 eq, HCl) in EtOH (0.2 mL) was added TEA (50.90 mg, 503.00 umol, 70.01 uL, 4 eq) was stirred at 25° C. for 15 min, and then was added AcOH (45.31 mg, 754.50 umol, 43.15 uL, 6 eq) and intermediate 223-5 (40.17 mg, 125.75 umol, 1 eq), the mixture was stirred at 25° C. for 15 min, followed by addition of NaBH3CN (15.80 mg, 251.50 umol, 2 eq). The resulting mixture was stirred at 25° C. for 12 hrs. LCMS showed Reactant 1 was consumed completely and one main peak with desired mass was detected. The mixture was filtered and concentrated under reduced pressure to give a residue. The residue was purified by prep-HPLC (column: Phenomenex luna C18 150*25 mm*10 um; mobile phase: [water (FA)-ACN]; B %: 30%-60%, 10 min) and the eluent was lyophilized to give Compound 144 (24 mg, 25.11 umol, 19.97% yield, 99.3% purity, FA) as off-white solid and confirmed by LCMS, HNMR, SFC.

### Mass Data

LCMS: Retention time: 0.482 min, (M+H)=903.7

LCMS: Retention time: 0.491 min, (M+H)=903.8

### NMR Data

¹H NMR (400 MHz, DMSO-d6) δ=8.39-8.35 (m, 1H), 8.18 (s, 1H), 8.06-7.95 (m, 2H), 7.87 (d, J=10.0 Hz, 1H), 7.74 (s, 1H), 7.54 (d, J=8.0 Hz, 2H), 7.48 (d, J=8.4 Hz, 1H), 7.43-7.36 (m, 2H), 7.33 (d, J=8.0 Hz, 3H), 7.20-7.11 (m, 4H), 7.10-6.98 (m, 2H), 4.42-4.32 (m, 2H), 4.25 (d, J=4.8 Hz, 2H), 3.98 (s, 3H), 3.92-3.80 (m, 4H), 3.58-3.51 (m, 1H), 3.16-3.04 (m, 3H), 2.83-2.71 (m, 1H), 2.65-2.55 (m, 2H), 2.51-2.50 (m, 1H), 2.48 (d, J=7.2 Hz, 2H), 2.20-2.13 (m, 5H), 1.76-1.67 (m, 2H), 1.47-1.26 (m, 2H), 1.24-1.19 (m, 5H), 1.11 (d, J=6.8 Hz, 3H).

### SFC Data

SFC: Retention time: 0.686, OD-3-MeOH+CAN (DEA)-60-3 mL-35T

### Example 224. Synthesis of [6-[[9-ethyl-7-(4-methyl-2-thienyl)carbazol-3-yl]methylamino]-N-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methyl]hexanamide] (Compound 145)

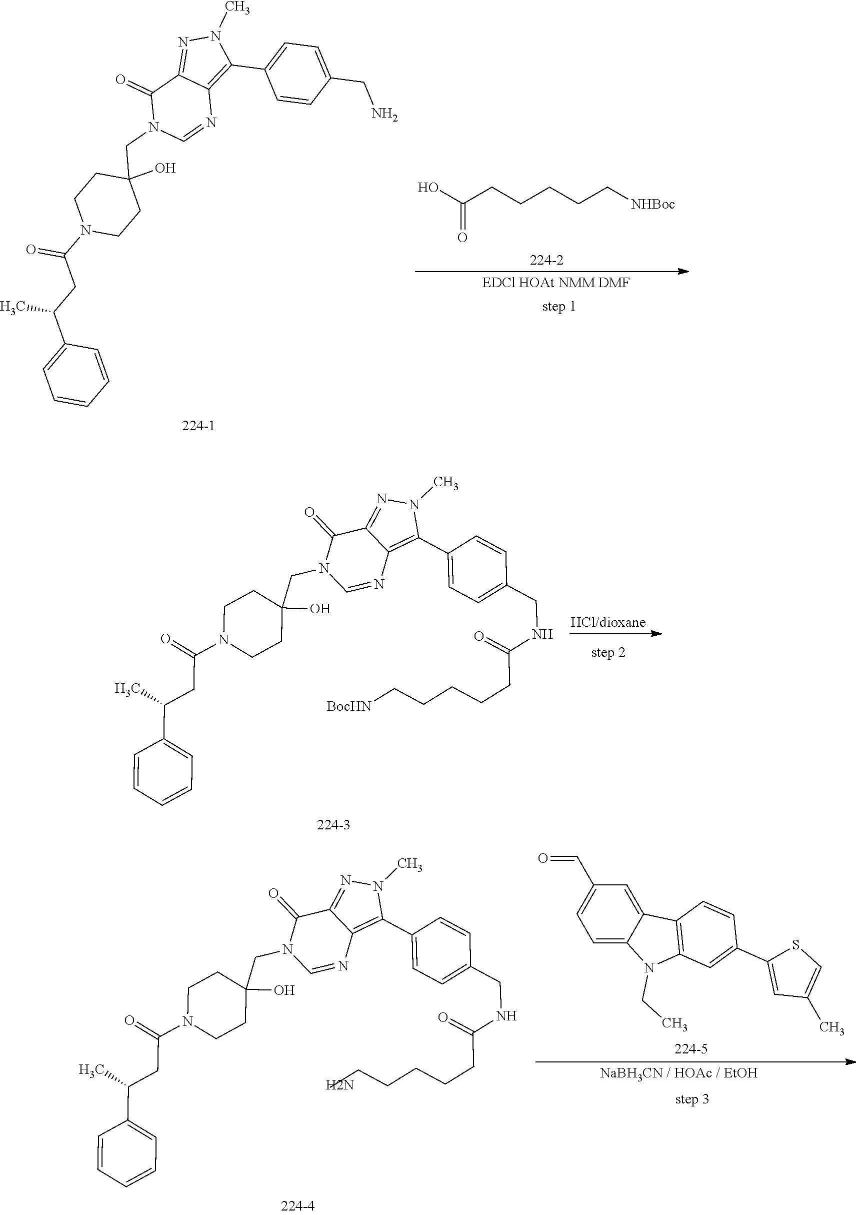

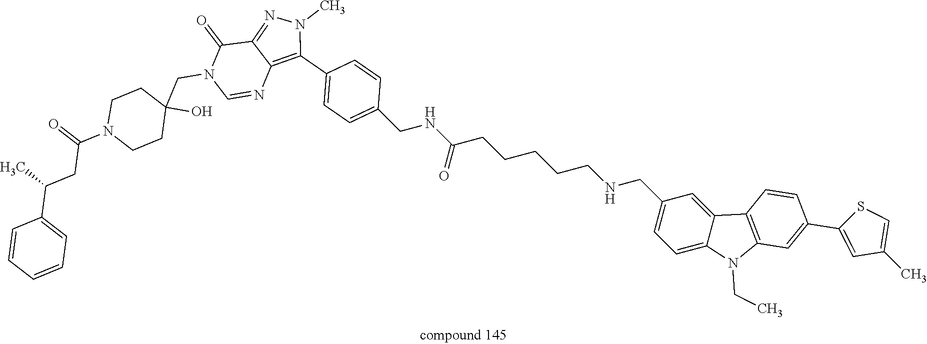 [SMILES: CCn1c2ccc(CNCCCCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 224-3

Detailed Synthetic Procedure: To a solution of Intermediate 224-2 (43.15 mg, 186.55 umol, 1.2 eq) in DMF (1 mL) was added EDCI (149.01 mg, 777.28 umol, 5 eq), NMM (157.24 mg, 1.55 mmol, 170.91 uL, 10 eq) and HOAT (42.32 mg, 310.91 umol, 43.49 uL, 2 eq), the mixture was stirred at 25° C. for 15 min. Then the Intermediate 224-1 (80 mg, 155.46 umol, 1 eq) was added into the mixture, the mixture was stirred at 25° C. for 1.5 hr. LCMS (EC3404-272-P1A1) showed 91% of desired mass was detected. (SiO2, by UV 254 nm, DCM:MeOH=10:1, Rf=0.6). The reaction mixture was washed with H2O (2 mL) at 25° C., and mixture was extracted with EA 9 ml (3 ml*3), and combined organic phase was dried with anhydrous sodium sulfate, filtered and concentrated to give crude product. The crude product was purified by column chromatography (SiO2, DCM/MeOH=1/0 to 0/1) and the eluent was concentrated under reduced pressure to give Intermediate 224-3 (90 mg, 119.94 umol, 77.15% yield, 97% purity) as a brown solid which was confirmed by LCMS.

### Mass Found

LCMS: Retention time: 0.451 min, (M+H)=728.6

LCMS: Retention time: 0.454 min, (M+H)=728.6

### Step 2: Synthesis of Intermediate 224-4

Detailed Synthetic Procedure: The Intermediate 224-3 (90 mg, 123.65 umol, 1 eq) was added in solution of HCl/dioxane (1 mL, 4 M). The mixture was stirred at 25° C. for 1 h. LCMS showed 95% of desired mass was detected. The mixture was concentrated under reduced pressure to give Intermediate 224-4 (90 mg, crude, HCl) as a white solid.

### Mass Found

LCMS: Retention time: 0.345 min, (M+H)=628.4

### Step 3: Synthesis of Compound 145

Detailed Synthetic Procedure: To a solution of Intermediate 224-4 (40 mg, 63.72 umol, 1 eq) in MeOH (0.5 mL) was added TEA (25.79 mg, 254.87 umol, 35.47 uL, 4 eq). The mixture was stirred at 25° C. for 15 min. Then NaBH3CN (24.02 mg, 382.30 umol, 6 eq), Intermediate 224-5 (20.35 mg, 63.72 umol, 1 eq) and AcOH (22.96 mg, 382.30 umol, 21.86 uL, 6 eq) was added into the mixture, the mixture was stirred at 25° C. for 12 hr. LCMS showed 57% of desired mass was detected. The reaction mixture was filtered and filtrate was concentrated under reduced pressure to give a residue. The crude product was purified by prep-HPLC (column: Phenomenex luna C18 150*25 mm*10 um;mobile phase: [water(FA)-ACN];B %: 27%-57%, 10 min) and the eluent was lyophilized to give Compound 145 (12.71 mg, 13.01 umol, 20.41% yield, 100% purity, FA) as off-white solid which was confirmed by HNMR, LCMS and SFC.

### Mass Found

LCMS: Retention time: 0.462 min, (M+H)=931.9

LCMS: Retention time: 0.493 min, (M+H)=931.4

### SFC Found

SFC: Retention time: 1.222 min, AD-3-IPA+CAN (DEA)-60-3 mL-35T.

### NMR Data

1H NMR (400 MHz, DMSO-d6) δ=8.40-8.37 (m, 1H), 8.34-8.28 (m, 1H), 8.12-8.06 (m, 2H), 7.96 (d, J=10.0 Hz, 1H), 7.83 (s, 1H), 7.64 (d, J=8.0 Hz, 2H), 7.57-7.53 (m, 1H), 7.50-7.45 (m, 2H), 7.45-7.41 (m, 3H), 7.28-7.23 (m, 4H), 7.16-7.13 (m, 1H), 7.12 (s, 1H), 4.49-4.46 (m, 2H), 4.34 (d, J=5.6 Hz, 2H), 4.07 (s, 3H), 3.97-3.89 (m, 4H), 3.65-3.60 (m, 1H), 3.21-3.12 (m, 3H), 2.91-2.85 (m, 1H), 2.67-2.64 (m, 2H), 2.57 (d, J=8.0 Hz, 2H), 2.28 (s, 3H), 2.20-2.15 (m, 2H), 1.59-1.51 (m, 4H), 1.34-1.30 (m, 9H), 1.20 (d, J=6.8 Hz, 3H)

### Example 225. Synthesis of [8-[[9-ethyl-7-(4-methyl-2-thienyl)carbazol-3-yl]methylamino]-N-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methyl]octanamide] (Compound 146)

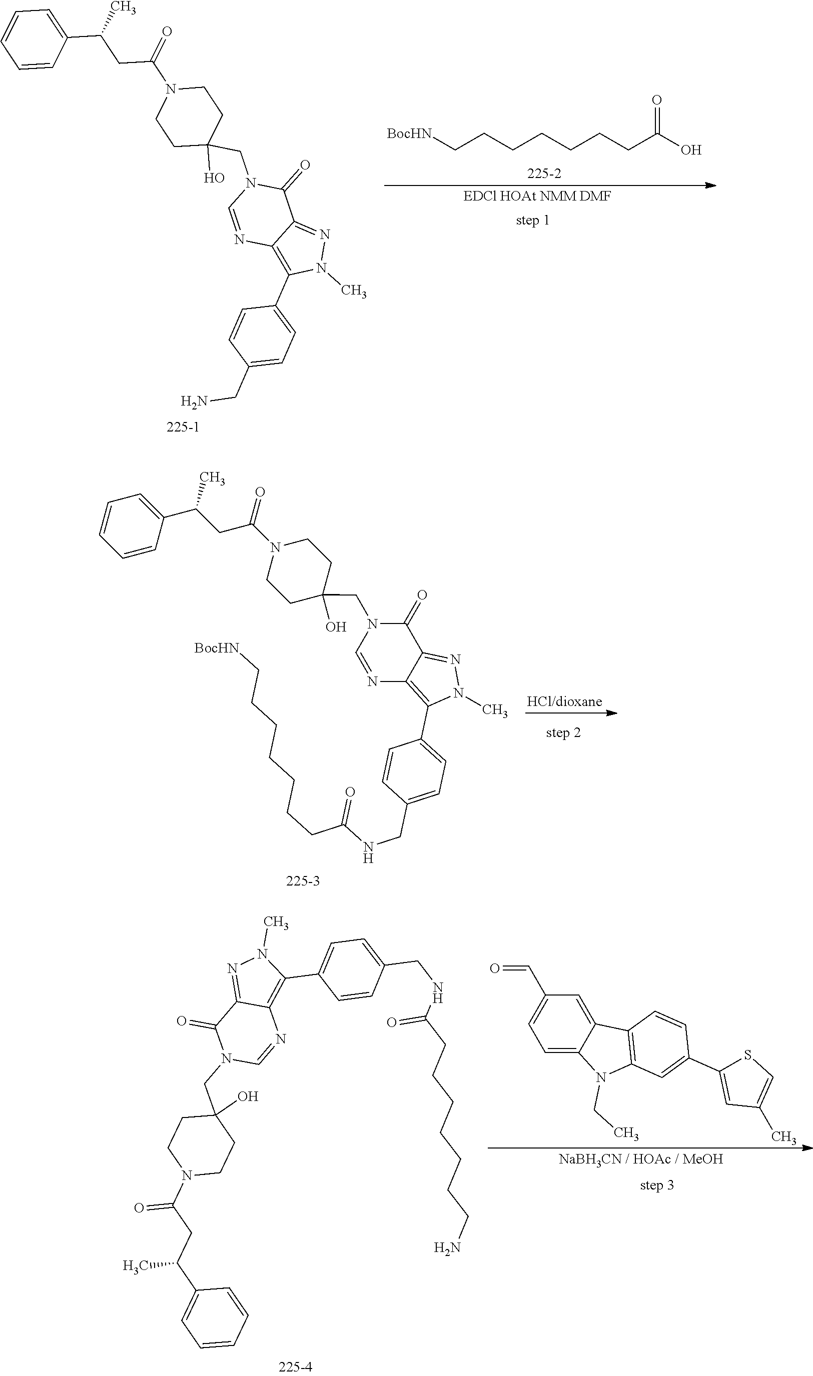 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCCCCCCC(=O)O ; C[C@H](CC(=O)N1CCC(O)(Cn2cnc3c(-c4ccc(CNC(=O)CCCCCCCNC(=O)OC(C)(C)C)cc4)n(C)nc3c2=O)CC1)c1ccccc1 ; C[C@H](CC(=O)N1CCC(O)(Cn2cnc3c(-c4ccc(CNC(=O)CCCCCCCN)cc4)n(C)nc3c2=O)CC1)c1ccccc1 ; CCn1c2ccc(C=O)cc2c2ccc(-c3cc(C)cs3)cc21]

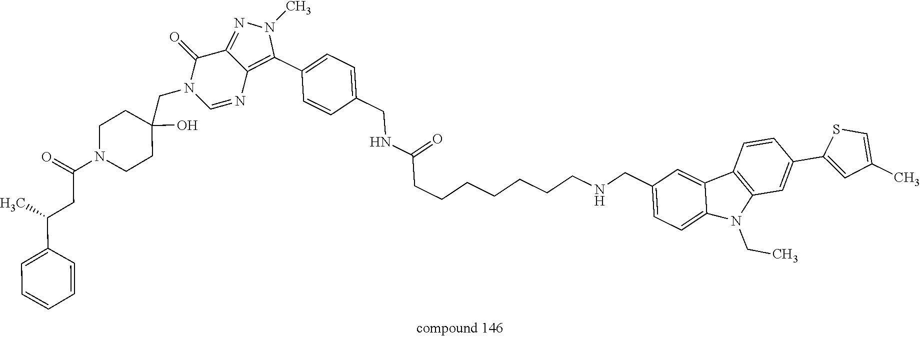 [SMILES: CCn1c2ccc(CNCCCCCCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 225-3

Detailed Synthetic Procedure: To a solution of intermediate 225-2 (60.47 mg, 233.18 umol, 1.2 eq) in DMF (1 mL) was added EDCI (186.26 mg, 971.60 umol, 5 eq) HOAt (52.90 mg, 388.64 umol, 54.37 uL, 2 eq) and NN (196.55 mg, 1.94 mmol, 213.64 uL, 10 eq) at 25° C. After addition, the mixture was stirred at this temperature for 0.5 hr, and then intermediate 225-1 (100 mg, 194.32 umol, 1 eq) was added at 25° C. The resulting mixture was stirred at 25° C. for 1.5 hr. LCMS showed the starting material was consumed completely and one major peak with desired mass was detected. The mixture was washed with water (2 mL) and extract with DCM (3 mL*3). The combined organic layers dried over sodium sulfate, filtered and concentrated under reduced pressure to give a residue. The residue was purified by column chromatography (SiO2, DCM/MeOH=1/0 to 0/1, Rf=0.50, DCM:MeOH=10:1) to get intermediate 225-3 (130 mg, 142.74 umol, 73.45% yield, 83% purity) as a brown solid by LCMS.

### Mass Found:

LCMS: Rt=0.428 min, (M+H)=756.6

LCMS: Rt=0.480 min, (M-100+H)=656.4

### Step 2: Synthesis of Intermediate 225-4

Detailed Synthetic Procedure: To a solution of intermediate 225-3 (80 mg, 105.83 umol, 1 eq) in dioxane (0.4 mL) was added HCl/dioxane (4 M, 0.8 mL). The mixture was stirred at 25° C. for 0.5 hr. LCMS showed the starting material was consumed completely and one major peak with desired mass was detected. The reaction mixture was concentrated in vacuo. The crude product intermediate 225-4 (80 mg, crude, HCl) was brown oil and it was used into the next step without further purification.

### Mass Found:

LCMS: Rt=0.358 min, (M+H)=656.5

### Step 3: Synthesis of Compound 146

Detailed Synthetic Procedure: To a solution of intermediate 225-4 (80 mg, 115.56 umol, 1 eq, HCl) in MeOH (0.8 mL) was added TEA (46.77 mg, 462.24 umol, 64.34 uL, 4 eq). The mixture was stirred at 25° C. for 0.2 min. Then NaBH3CN (43.57 mg, 693.35 umol, 6 eq), intermediate 225-5 (18.46 mg, 57.78 umol, 0.5 eq) and AcOH (41.64 mg, 693.35 umol, 39.65 uL, 6 eq) was added into the mixture, the mixture was stirred at 25° C. for 1.8 hr. LCMS showed the starting material was consumed completely and 42% of desired mass was detected. The reaction mixture was concentrated in vacuo. The residue was purified by prep-HPLC (column: Phenomenex luna C18 150*25 mm*10 um;mobile phase: [water(FA)-ACN];B %: 30%-60%, 10 min) and the eluent was lyophilized to give compound 146 (15 mg, 15.59 umol, 13.49% yield, 99.685% purity) as an off-white solid by LCMS, HNMR and SFC.

### Mass Found:

LCMS: Rt=0.509 min, (M+H)=959.8

LCMS: Rt=0.509 min, (M+H)=959.7

### SFC Data:

SFC: Rt=1.116 min, OD-3-IPA+ACN (DEA)-60-3 mL-35T

### HNMR Data:

¹H NMR (400 MHz, DMSO-d6) δ=8.37-8.35 (m, 1H), 8.24 (s, 1H), 8.14-8.08 (m, 2H), 7.96 (d, J=9.6 Hz, 1H), 7.85 (s, 1H), 7.65 (d, J=7.6 Hz, 2H), 7.59 (d, J=8.0 Hz, 1H), 7.49 (s, 2H), 7.45-7.40 (m, 3H), 7.25 (d, J=4.0 Hz, 4H), 7.15 (s, 1H), 7.13 (s, 1H), 4.52-4.46 (m, 2H), 4.34 (d, J=5.6 Hz, 2H), 4.08 (s, 3H), 4.04 (s, 2H), 3.98 (d, J=8.8 Hz, 1H), 3.90 (d, J=6.4 Hz, 1H), 3.67-3.60 (m, 1H), 3.21-3.09 (m, 3H), 2.92-2.84 (m, 1H), 2.73-2.72 (m, 2H), 2.61-2.54 (m, 2H), 2.27 (s, 3H), 2.16-2.14 (m, 2H), 1.53 (d, J=4.4 Hz, 4H), 1.36-1.24 (m, 13H), 1.20 (d, J=6.8 Hz, 3H).

### Example 226. Synthesis of 3-[2-[2-[[9-ethyl-7-(4-methyl-2-thienyl)carbazol-3-yl]methylamino]ethoxy]ethoxy]-N-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methyl]propanamide (Compound 147)

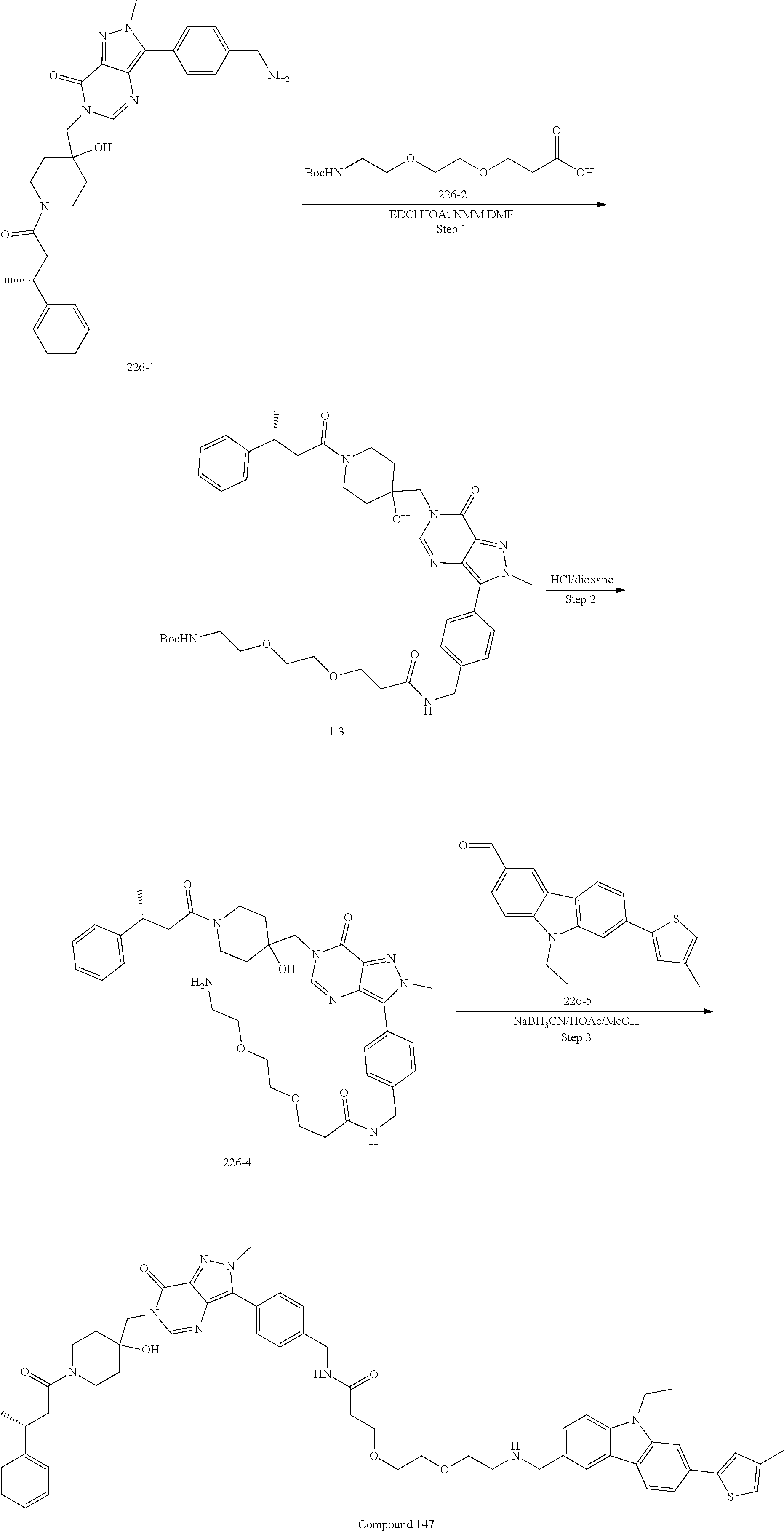 [SMILES: C[C@H](CC(=O)N1CCC(O)(Cn2cnc3c(-c4ccc(CN)cc4)n(C)nc3c2=O)CC1)c1ccccc1 ; CC(C)(C)OC(=O)NCCOCCOCCC(=O)O ; C[C@H](CC(=O)N1CCC(O)(Cn2cnc3c(-c4ccc(CNC(=O)CCOCCOCCNC(=O)OC(C)(C)C)cc4)n(C)nc3c2=O)CC1)c1ccccc1 ; C[C@H](CC(=O)N1CCC(O)(Cn2cnc3c(-c4ccc(CNC(=O)CCOCCOCCN)cc4)n(C)nc3c2=O)CC1)c1ccccc1 ; CCn1c2ccc(C=O)cc2c2ccc(-c3cc(C)cs3)cc21 ; CCn1c2ccc(CNCCOCCOCCC(=O)NCc3ccc(-c4c5ncn(CC6(O)CCN(C(=O)C[C@@H](C)c7ccccc7)CC6)c(=O)c5nn4C)cc3)cc2c2ccc(-c3cc(C)cs3)cc21]

### Step 1: Synthesis of Intermediate 226-3

To a solution of intermediate 226-1 (30 mg, 58.30 umol, 1 eq) and intermediate 226-2 (24.25 mg, 87.44 umol, 1.5 eq) in DMF (0.3 mL) was added NN (29.48 mg, 291.48 umol, 32.05 uL, 5 eq), EDCI (55.88 mg, 291.48 umol, 5 eq) and HOAt (11.90 mg, 87.44 umol, 12.23 uL, 1.5 eq). The mixture was stirred at 25° C. for 2 hr. LC-MS showed 81.5% of desired compound was detected. TLC (PE:EA=1:1, by UV=254 nm) showed two new main spots (Rf=0.35, 0.45) was observed. The reaction mixture was washed with water (10 mL) and extracted with EA (25 mL*3), the combined organic phase was dried by Na2SO4, concentrated under reduced pressure to give a residue. The residue was purified by flash silica gel chromatography (ISCO®; 20 g SepaFlash® Silica Flash Column, Eluent of 0˜50% Methanol/Dichloromethane @ 20 mL/min) and the eluent was concentrated to give product. Intermediate 226-3 (31 mg, 37.44 umol, 64.23% yield, 93.47% purity) was obtained as a brown solid and confirmed by SFC. LCMS showed 93.47% desired product was detected.

### Mass Found:

LCMS: Retention time=0.443 min, M+H=674.4

LCMS: Retention time=0.543 min, M+H=674.2

### SFC Found:

SFC: Retention time: 1.207 min, OD-3-MeOH+ACN (DEA)-40-3 mL-35T.

### Step 2: Synthesis of Intermediate 226-4

The solution of Intermediate 226-3 (78 mg, 100.79 umol, 1 eq) in HCl/dioxane (0.8 mL, 4 M) was stirred at 25° C. for 2 hr. LC-MS showed 95.16% of desired compound was detected. Concentrated under reduced pressure to give a residue. The crude product was used into the next step without further purification. Intermediate 226-4 (70 mg, 93.79 umol, 93.06% yield, 95.16395.163% purity, HCl) was obtained as an orange oil.

### Mass Found

LCMS: Retention time: 0.346 min, (M+H)=674.4, 5-95AB_0.8 min

### Step 3: Synthesis of Compound 147

Detailed Synthetic Procedure: To a solution of Intermediate 226-4 (50 mg, 74.21 umol, 1 eq) in MeOH (0.3 mL) was added TEA (30.04 mg, 296.82 umol, 41.31 uL, 4 eq) at 25° C., the mixture was stirred at 25° C. for 10 min. Then HOAc (26.74 mg, 445.24 umol, 25.46 uL, 6 eq), Intermediate 226-5 (18.96 mg, 59.36 umol, 0.8 eq) was added and the mixture was stirred at 25° C. for 30 min. Then NaBH3CN (27.98 mg, 445.24 umol, 6 eq) were added at 25° C. The resulting mixture was stirred at 25° C. for 3 hr. LC-MS showed 61.41% of desired mass was detected. The reaction mixture was filtered and the filtrate was purified by flash silica gel chromatogr. The crude was purified by prep-HPLC (column: Phenomenex luna C18 150*25 mm*10 um; mobile phase: [water (FA)-ACN]; B %: 27%-57%, 10 min) and the eluent was concentrated to remove ACN and lyophilized to give product. Compound 147 (15 mg, 15.08 umol, 20.32% yield, 98.218% purity) was obtained as an off-white solid and confirmed by 1H NMR and LCMS and SFC.

### Mass Found

LCMS: Retention time=0.604 min, M+H=977.5

LCMS: Retention time=0.491 min, M+H=977.8

### SFC Found

SFC: Retention time: D-3-IPA+ACN DEA)-60-3 mL-35T

### NMR Data

1H NMR (400 MHz, CHLOROFORM-d) δ=7.98-7.79 (m, 3H), 7.59-7.53 (m, 1H), 7.48 (d, J=8.0 Hz, 1H), 7.40-7.30 (m, 6H), 7.28-7.13 (m, 6H), 6.91 (s, 1H), 4.44 (d, J=5.6 Hz, 2H), 4.38-4.20 (m, 3H), 4.17-4.06 (m, 2H), 3.96 (s, 3H), 3.84 (s, 1H), 3.79-3.76 (m, 2H), 3.70-3.59 (m, 6H), 3.55 (d, J=14.0 Hz, 1H), 3.36-3.21 (m, 3H), 3.01-2.89 (m, 3H), 2.66-2.58 (m, 1H), 2.54-2.43 (m, 3H), 2.34 (s, 3H), 1.58-1.50 (m, 1H), 1.48-1.39 (m, 5H), 1.37-1.29 (m, 4H).

### Example 227. Synthesis of [3-[4-[[[9-ethyl-7-(4-methyl-2-thienyl) carbazol-3-yl]methylamino] methyl] phenyl]-6-[[4-hydroxy-1-[(3R)-3-phenylbutanoyl]-4-piperidyl]methyl]-2-methyl-pyrazolo [4, 3-d] pyrimidin-7-one] (Compound 148)

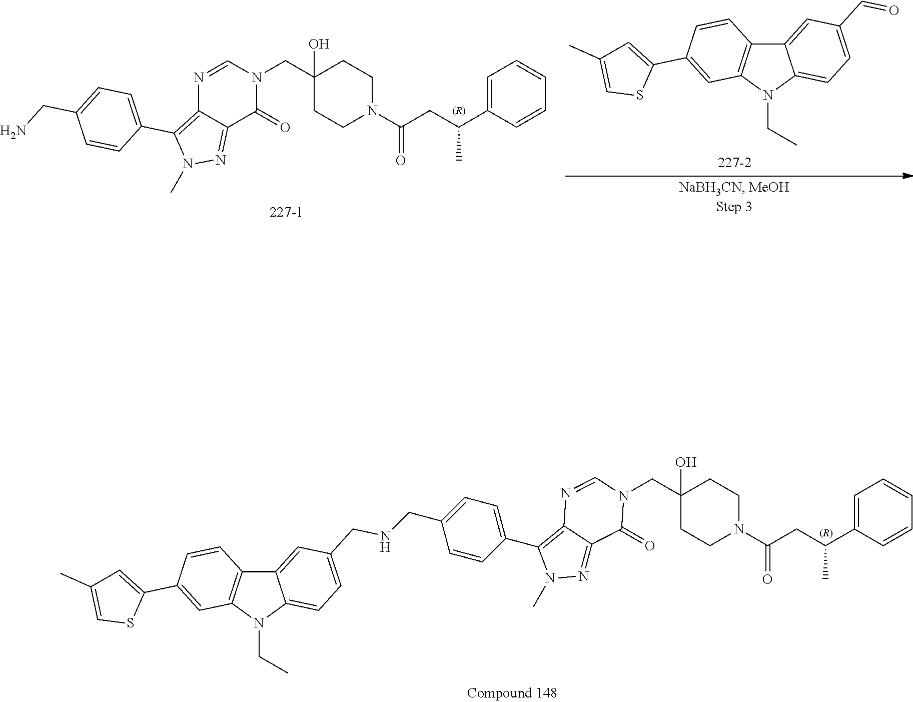

### Step 1: Synthesis of Compound 148

Detailed Synthetic Procedure: To a solution of intermediate 227-1 (30.00 mg, 58.30 umol, 1 eq) in MeOH (0.3 mL) was added TEA (23.60 mg, 233.18 umol, 32.46 uL, 4 eq) stirred at 25° C. for 10 min, then the intermediate 227-2 (18.62 mg, 58.30 umol, 1 eq) and AcOH (21.00 mg, 349.77 umol, 20.00 uL, 6 eq) was added in, stirred at 25° C. for 10 min and the NaBH3CN (21.98 mg, 349.77 umol, 6 eq) was added in. The mixture was stirred at 25° C. for 12 h. LCMS showed intermediate 227-1 was consumed and desired mass was detected. The reaction was concentrated under reduced pressure to give a residue. The residue was purified by prep-HPLC (column: Phenomenex luna C18 150*25 mm*10 um; mobile phase: [water (FA)-ACN]; B %: 30%-60%, 10 min). The eluent was lyophilization to give product. Compound 148 (8 mg, 9.68 umol, 16.61% yield, 99% purity) was obtained as an off-white solid. It was confirmed by LCMS, HNMR, SFC.

### Mass Found

LCMS: Retention time: 0.715 min, (M+H)=818.4

LCMS: Retention time: 0.492 min, (M+H)=818.3

### SFC Data

SFC: Retention time: 0.536 min, AS-3-IPA+CAN (DEA)-60-3 mL-35T.

### NMR Data

1H NMR (400 MHz, DMSO-d6) δ=8.25 (s, 1H), 8.17-8.08 (m, 2H), 7.98 (d, J=10.0 Hz, 1H), 7.83 (s, 1H), 7.68 (d, J=8.0 Hz, 2H), 7.61-7.54 (m, 3H), 7.51-7.47 (m, 2H), 7.42 (d, J=8.4 Hz, 1H), 7.29-7.23 (m, 4H), 7.18-7.11 (m, 2H), 4.90-4.83 (m, 1H), 4.51-4.45 (m, 2H), 4.11 (s, 3H), 4.06-3.97 (m, 2H), 3.91 (s, 2H), 3.85 (s, 2H), 3.74-3.57 (m, 2H), 3.21-3.16 (m, 2H), 2.93-2.83 (m, 1H), 2.63-2.53 (m, 2H), 2.28 (s, 3H), 1.40-1.29 (m, 6H), 1.21 (d, J=6.8 Hz, 4H).

### Example 228. Synthesis of [[(R,E)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)-2-(2-(5-oxo-3-phenyl-1-(4-phenylthiazol-2-yl)-1H-pyrazol-4(5H)-ylidene)hydrazinyl)thiazole-5-carboxamide]] (Compound 149)

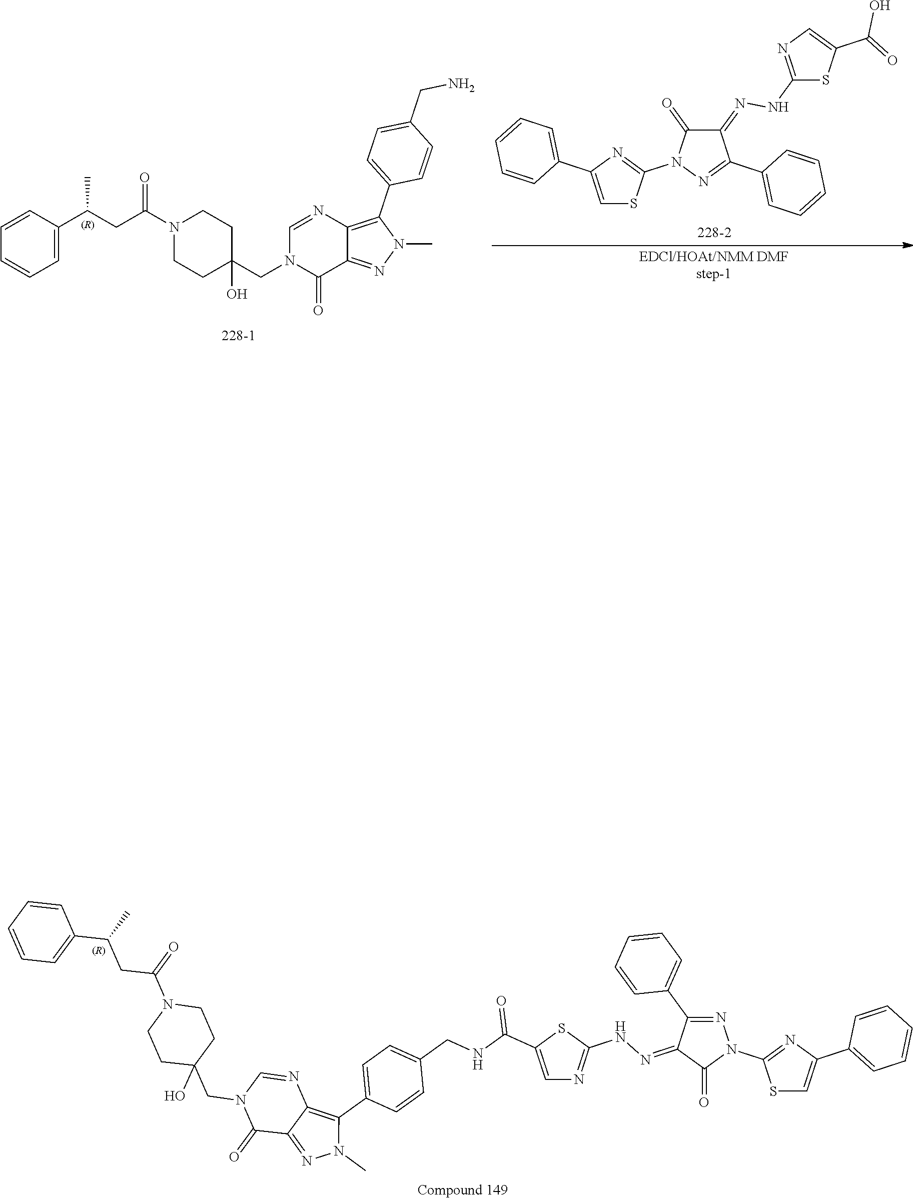

### Step 1: Synthesis of Compound 149

Detailed Synthetic Procedure: To a solution of intermediate 228-1 (30.00 mg, 58.30 umol, 1 eq), intermediate 228-2 (33.19 mg, 69.95 umol, 1.2 eq) in DMF (0.3 mL) was added EDCI (22.35 mg, 116.59 umol, 2.0 eq) and HOAt (7.93 mg, 58.30 umol, 8.15 uL, 1 eq) and NMM (29.48 mg, 291.48 umol, 32.05 uL, 5 eq). The mixture was stirred at 25° C. for 1 hr. LC-MS showed Reactant 1 was consumed completely and one major peak with desired mass was detected. The reaction mixture was diluted with MeOH (0.3 ml) and then submitted for by prep-HPLC purification directly (column: Phenomenex C18 150*25 mm*10 um; mobile phase: [water (NH4HCO3)-ACN]; B %: 30%-60%, 8 min), the eluent was concentrated to remove ACN and lyophilized to give product Compound 149 (6.5 mg, 5.99 umol, 10.28% yield, 100% purity, TFA) as an orange solid which was confirmed by HNMR, FNMR, SFC, and LCMS

### Mass:

Retention time: 0.591 min, (M+H)=971.4

Retention time: 0.567 min, (M+H)=971.2

### SFC:

Retention time: 1.093 min, OJ-3-IPA+ACN (DEA)-50-3 mL-35T.

### NMR Data:

1H NMR (400 MHz, DMSO-d6) δ=9.31-9.09 (m, 1H), 8.28 (s, 1H), 8.17 (d, J=7.6 Hz, 2H), 8.02-7.96 (m, 3H), 7.81 (s, 1H), 7.70 (d, J=8.4 Hz, 2H), 7.62-7.51 (m, 5H), 7.49-7.44 (m, 2H), 7.39-7.32 (m, 1H), 7.29-7.23 (m, 4H), 7.19-7.12 (m, 1H), 4.56 (d, J=5.6 Hz, 2H), 4.11 (s, 3H), 4.06 (s, 2H), 3.98-3.90 (m, 2H), 3.25-3.16 (m, 2H), 2.91-2.83 (m, 1H), 2.62-2.56 (m, 2H), 1.42-1.23 (m, 4H), 1.20 (d, J=7.2 Hz, 3H).

### Example 229. Synthesis of [[(E)-5-(2-(2-(5-oxo-3-phenyl-1-(4-phenylthiazol-2-yl)-1H-pyrazol-4(5H)-ylidene)hydrazinyl)thiazole-5-carbonyl)-N-(6-(thiophene-2-sulfonamido)benzo[d]thiazol-2-yl)-5,6,7,8-tetrahydro-4H-pyrazolo[1,5-a][1,4]diazepine-2-carboxamide]] (Compound 150)

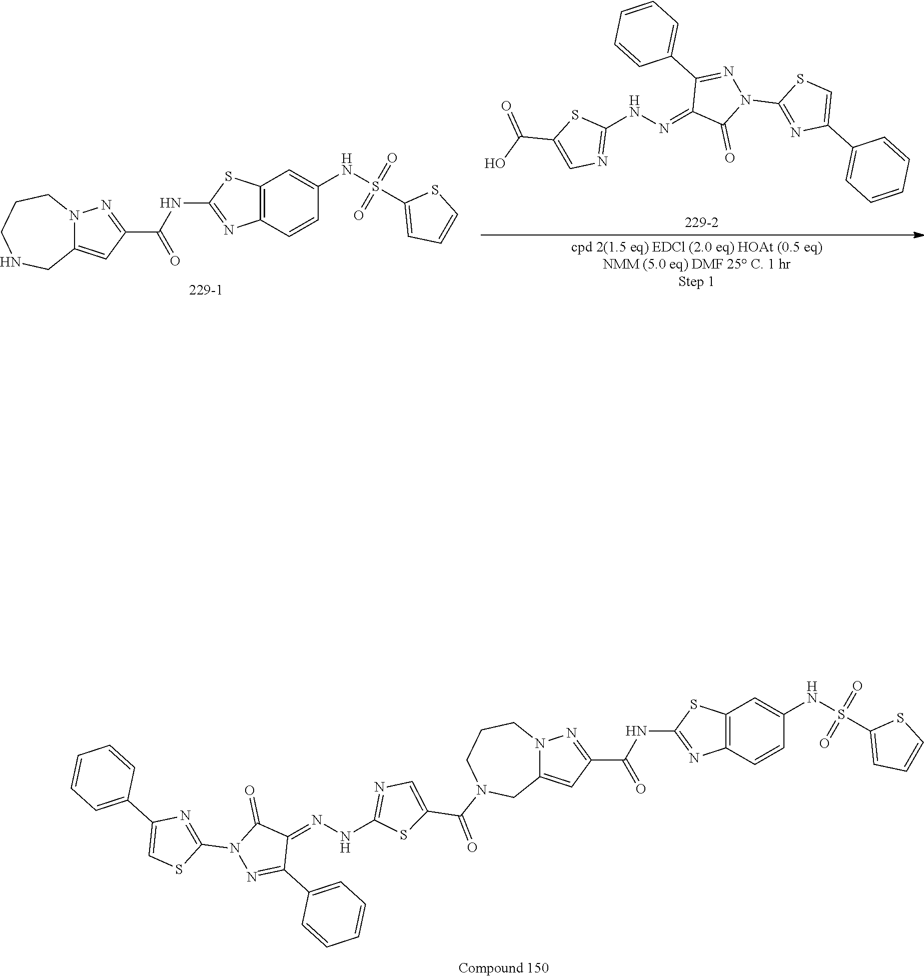 [SMILES: O=C(Nc1nc2ccc(NS(=O)(=O)c3cccs3)cc2s1)c1cc2n(n1)CCCNC2 ; O=C(O)c1cnc(N/N=C2/C(=O)N(c3nc(-c4ccccc4)cs3)N=C2c2ccccc2)s1 ; O=C(Nc1nc2ccc(NS(=O)(=O)c3cccs3)cc2s1)c1cc2n(n1)CCCN(C(=O)c1cnc(N/N=C3/C(=O)N(c4nc(-c5ccccc5)cs4)N=C3c3ccccc3)s1)C2]

### Step 1: Synthesis of Compound 150

Detailed Synthetic Procedure: To a solution of intermediate 229-1 (0.05 g, 105.36 umol, 1 eq) and intermediate 229-2 (74.99 mg, 158.03 umol, 1.5 eq) in DMF (0.5 mL) was added EDCI (40.39 mg, 210.71 umol, 2 eq), HOAt (7.17 mg, 52.68 umol, 7.37 uL, 0.5 eq) and NN (53.28 mg, 526.78 umol, 57.92 uL, 5 eq), the mixture was stirred at 25° C. for 1 hr. LCMS showed 54.82% desired mass was detected. The mixture was filtered to give a residue. The residue was purified by preparative HPLC (column: Phenomenex luna C18 150*25 mm*10 um; mobile phase: [water (TFA)-ACN]; B %: 60%-90%, 10 min) and lyophilized to give compound 150 (7 mg, 7.52 umol, 7.14% yield, 100% purity) as an orange solid which was confirmed by LCMS, HNMR and FNMR.

### Mass Found

LCMS: Retention time: 0.568 min, (M+H)=931.4

LCMS: Retention time: 0.589 min, (M+H)=931.5

### NMR Data

1H NMR (400 MHz, DMSO-d6) δ=12.38-12.12 (m, 1H), 10.43 (s, 1H), 8.15 (d, J=7.2 Hz, 2H), 8.03-7.99 (m, 3H), 7.89-7.85 (m, 1H), 7.81 (s, 1H), 7.74 (d, J=2.0 Hz, 1H), 7.66 (d, J=8.8 Hz, 1H), 7.57-7.52 (m, 3H), 7.47 (t, J=7.6 Hz, 3H), 7.38-7.34 (m, 1H), 7.35-7.17 (m, 1H), 7.12-7.08 (m, 1H), 7.04 (s, 1H), 4.96-4.88 (m, 2H), 4.65-4.59 (m, 2H), 4.05 (s, 2H), 2.08 (s, 2H).

### Example 230. Synthesis of [3-[4-[[4-[[9-ethyl-7-(4-methyl-2-thienyl)carbazol-3-yl]methylamino]-1-piperidyl]methyl]phenyl]-6-[[4-hydroxy-1-[(3R)-3-phenylbutanoyl]-4-piperidyl]methyl]-2-methyl-pyrazolo[4,3-d]pyrimidin-7-one] (Compound 151)

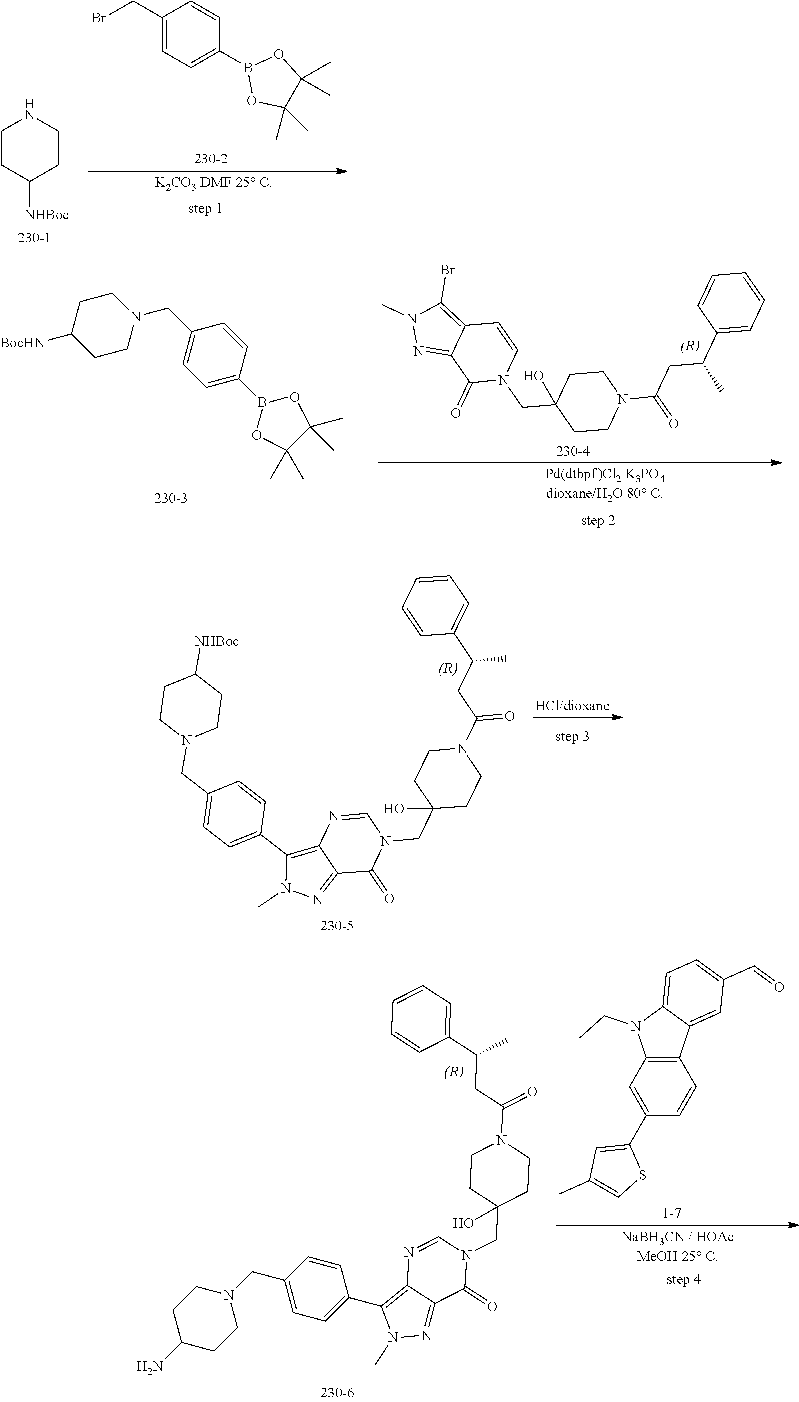

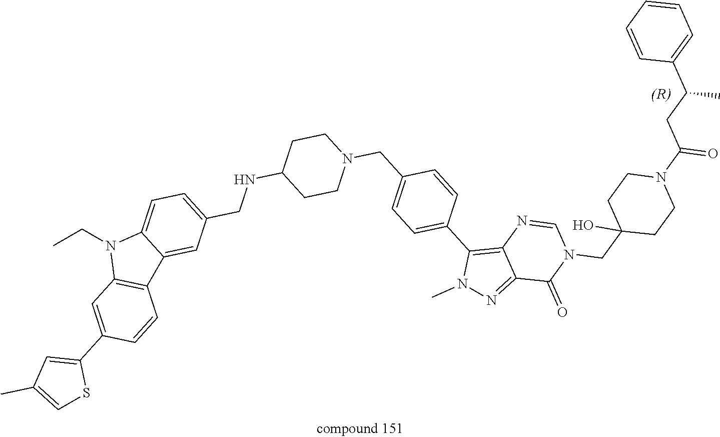

### Step 1: Synthesis of Intermediate 230-3

Detailed Synthetic Procedure: To a solution of intermediate 230-1 (500 mg, 2.50 mmol, 1 eq) and intermediate 230-1 (889.75 mg, 3.00 mmol, 1.2 eq) in DMF (5 mL) was added K2CO3 (1.04 g, 7.49 mmol, 3 eq). The mixture was stirred at 25° C. for 2 hr. LCMS showed the starting material was consumed completely and 38% of desired mass was detected. The mixture was washed with water (5 mL) and extract with DCM (8 mL*3). The combined organic layers dried over sodium sulfate, filtered and concentrated under reduced pressure to give a residue. The intermediate 230-3 (500 mg, crude) was a white solid and it was used into the next step without further purification.

### Mass Found:

LCMS: Rt=0.400 min, (M+H)=417.0

### Step 2: Synthesis of Intermediate 230-5

Detailed Synthetic Procedure: A mixture of intermediate 230-3 (255.76 mg, 614.28 umol, 1.2 eq), intermediate 230-4 (250.00 mg, 511.90 umol, 1 eq), Pd(dtbpf)Cl2 (66.73 mg, 102.38 umol, 0.2 eq) and K3PO4 (325.98 mg, 1.54 mmol, 3 eq) in dioxane (2.5 mL) and H2O (0.5 mL) was degassed and purged with N2 for 3 times, and then the mixture was stirred at 80° C. for 2 hr under N2 atmosphere. LCMS showed the starting material was consumed completely and 42% of desired mass was detected. The mixture was washed with water (5 mL) and extract with DCM (8 mL*3). The combined organic layers was dried over sodium sulfate, filtered and concentrated under reduced pressure to give a residue. The residue was purified by column chromatography (SiO2, EA/MeOH=1/0 to 0/1, Rf=0.35, EA:MeOH=2:1) to give intermediate 230-5 (350 mg, 366.12 umol, 71.52% yield, 73% purity) as a black solid which was confirmed by LCMS.

### Mass Found:

LCMS: Rt=0.389 min, (M+H)=698.4

LCMS: Rt=0.389 min, (M+H)=698.4

### Step 3: Synthesis of Intermediate 230-6

Detailed Synthetic Procedure: To a solution of intermediate 230-5 (300 mg, 429.88 umol, 1 eq) in dioxane (3 mL) was added HCl/dioxane (4 M, 3.00 mL). The mixture was stirred at 25° C. for 0.5 hr. LCMS showed the starting material was consumed completely and 61% of desired mass was detected. The reaction mixture was concentrated in vacuo. The intermediate 230-6 (300 mg, crude, HCl) was a black solid and it was used into the next step without further purification.

### Mass Found:

LCMS: Rt=0.298 min, (M+H)=598.6

### Step 4: Synthesis of Compound 151

Detailed Synthetic Procedure: To a solution of intermediate 230-6 in MeOH (3 mL) was added TEA (203.14 mg, 2.01 mmol, 279.42 uL, 4 eq) at 25° C. for 10 min. Then intermediate 230-7 (128.25 mg, 401.51 umol, 0.8 eq) and HOAc (180.83 mg, 3.01 mmol, 172.22 uL, 6 eq) was added at 25° C. for 20 min. Then NaBH3CN (189.24 mg, 3.01 mmol, 6 eq) was added. The resulting mixture was stirred at 25° C. for 11.5 hr. LC-MS showed the starting material was consumed completely and 52% of desired mass was detected. The mixture was washed with water (5 mL) and extract with DCM (8 mL*3). The combined organic layers was dried over sodium sulfate, filtered and concentrated under reduced pressure to give a residue. The residue was purified by prep-HPLC (column: Waters xbridge 150*25 mm 10 um;mobile phase: [water(NH4HCO3)-ACN];B %: 58%-88%, 9 min) and the eluent was lyophilized to give compound 151 (60 mg, 66.37 umol, 13.22% yield, 99.687% purity) as a gray solid which was confirmed by LCMS, HNMR and SFC.

### Mass Found:

LCMS: Rt=0.756 min, (M+H)=901.9

LCMS: Rt=0.751 min, (M+H)=901.9

### SFC Data:

SFC: Rt=0.937 min, OD-3-IPA+ACN(DEA)-60-3 mL-35T.

### HNMR Data:

¹H NMR (400 MHz, DMSO-d6) δ=8.12 (d, J=8.0 Hz, 1H), 8.07 (s, 1H), 7.98 (d, J=10.0 Hz, 1H), 7.83 (s, 1H), 7.67 (d, J=8.0 Hz, 2H), 7.56-7.52 (m, 1H), 7.51-7.47 (m, 3H), 7.47-7.41 (m, 2H), 7.30-7.24 (m, 4H), 7.19-7.14 (m, 1H), 7.13 (s, 1H), 4.87 (d, J=4.8 Hz, 1H), 4.48-4.45 (m, 2H), 4.11 (s, 3H), 4.04-3.89 (m, 4H), 3.72-3.59 (m, 1H), 3.53 (s, 2H), 3.26-3.12 (m, 3H), 2.91-2.77 (m, 3H), 2.63-2.54 (m, 3H), 2.28 (s, 3H), 2.04-1.97 (m, 2H), 1.92-1.85 (m, 2H), 1.41-1.32 (m, 7H), 1.27-1.16 (m, 5H).

### Example 231. Synthesis of [N-[[4-[6-[[4-hydroxy-1-(3-phenylbutanoyl)-4-piperidyl]methyl]-2-methyl-7-oxo-pyrazolo[4,3-d]pyrimidin-3-yl]phenyl]methyl]-4-[2-[(4E)-5-oxo-3-phenyl-4-(thiazol-2-ylhydrazono)pyrazol-1-yl]thiazol-4-yl]benzamide] (Compound 152)

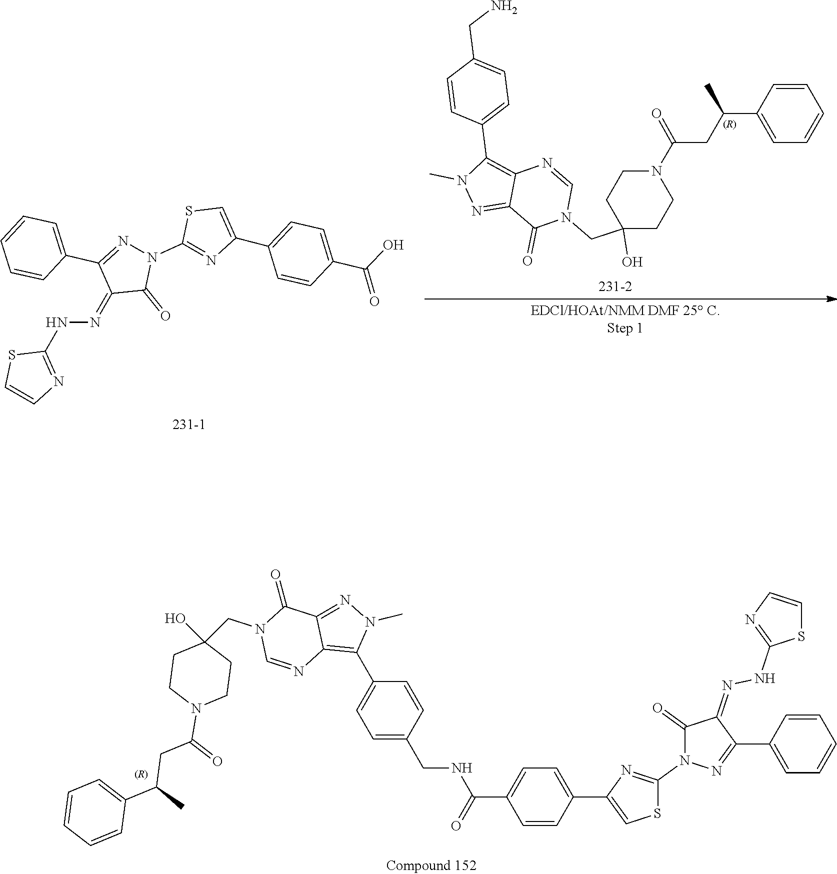

### Step 1: Synthesis of Compound 152

Detailed Synthetic Procedure: To a solution of intermediate 231-1 (30 mg, 63.22 umol, 1 eq) in DMF (0.5 mL) was added EDCI (36.36 mg, 189.66 umol, 3 eq) NMM (31.97 mg, 316.10 umol, 34.75 uL, 5 eq) and HOAt (12.91 mg, 94.83 umol, 13.27 uL, 1.5 eq), the mixture was stirred at 25° C. for 10 min and then the intermediate 231-2 (39.04 mg, 75.86 umol, 1.2 eq) was added and stirred at 25° C. for 12 h. LCMS showed intermediate 231-1 was consumed and desired mass was detected. The reaction mixture was diluted with water and orange precipitate was collected by filtration and then the obtained orange solid was purified by prep-HPLC (column: Phenomenex luna C18 150*25 mm*10 um;mobile phase: [water(TFA)-ACN];B %: 45%-75%, 10 min) The eluent was lyophilizated to give product Compound 1 (10 mg, 10.30 umol, 16.29% yield, 100% purity) as orange solid which was confirmed by LCMS, HNMR, FNMR, and SFC.

### Mass Found

LCMS: Retention time: 0.535 min, (M+H)=971.7

LCMS: Retention time: 0.536 min, (M+H)=971.3

### SFC Data

SFC: Retention time: 1.054 min, AS-3-MeOH+ACN (DEA)-60-3 mL-35T.

### NMR Data

1H NMR (400 MHz, DMSO-d6) δ=9.22-919 (m, 1H), 8.17 (d, J=6.4 Hz, 2H), 8.14-8.09 (m, 2H), 8.03 (d, J=8.4 Hz, 2H), 8.00-7.96 (m, 2H), 7.73-7.67 (m, 3H), 7.58-7.51 (m, 5H), 7.34 (d, J=4.0 Hz, 1H), 7.28-7.23 (m, 4H), 7.18-7.13 (m, 1H), 4.60 (d, J=4.8 Hz, 2H), 4.11 (s, 3H), 4.06-3.86 (m, 4H), 3.19-3.16 (m, 2H), 2.91-2.84 (m, 1H), 2.61-2.56 (m, 2H), 1.40-1.29 (m, 3H), 1.24-1.18 (m, 4H).

### Example 232. Synthesis of [5-[4-[2-[(4E)-5-oxo-3-phenyl-4-(thiazol-2-ylhydrazono)pyrazol-1-yl]thiazol-4-yl]benzoyl]-N-[6-(2-thienylsulfonylamino)-1,3-benzothiazol-2-yl]-4,6,7,8-tetrahydropyrazolo[1,5-a][1,4]diazepine-2-carboxamide] (Compound 153)

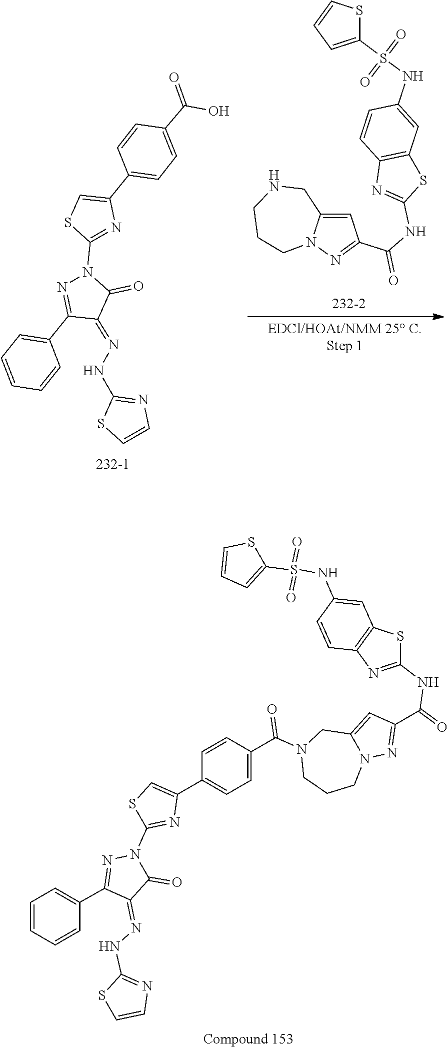 [SMILES: O=C(O)c1ccc(-c2csc(N3N=C(c4ccccc4)/C(=N\Nc4nccs4)C3=O)n2)cc1 ; O=C(Nc1nc2ccc(NS(=O)(=O)c3cccs3)cc2s1)c1cc2n(n1)CCCNC2 ; O=C(Nc1nc2ccc(NS(=O)(=O)c3cccs3)cc2s1)c1cc2n(n1)CCCN(C(=O)c1ccc(-c3csc(N4N=C(c5ccccc5)/C(=N\Nc5nccs5)C4=O)n3)cc1)C2]

### Step 1: Synthesis of Compound 153

Detailed Synthetic Procedure: To a solution of intermediate 232-1 (20 mg, 42.15 umol, 1 eq) in DMF (0.5 mL) was added EDCI (24.24 mg, 126.45 umol, 3 eq), HOAt (8.61 mg, 63.22 umol, 8.84 uL, 1.5 eq) and NMM (21.32 mg, 210.74 umol, 23.17 uL, 5 eq) stirred at 25° C. for 10 min and then the intermediate 232-2 (24.00 mg, 50.58 umol, 1.2 eq) was added in. The mixture was stirred at 25° C. for 2 hr. LCMS showed intermediate 232-1 was consumed and desired mass was detected. The mixture was filtered to give a residue. The residue was purified by prep-HPLC (column: Welch Ultimate C18 150*25 mm*5 um; mobile phase: [water(TFA)-ACN];B %: 47%-77%, 10 min). The eluent was lyophilization to give product. Compound 153 (9 mg, 9.47 umol, 22.48% yield, 98% purity) was obtained as an orange solid. It was confirmed by LCMS, HNMR, FNMR.

### Mass Found

LCMS: Retention time: 0.554 min, (M+H)=931.1

LCMS: Retention time: 0.541 min, (M+H+2)=931.0

### NMR Data

1H NMR (400 MHz, DMSO-d6) δ=12.44-12.21 (m, 1H), 10.44 (s, 1H), 8.16 (d, J=7.6 Hz, 2H), 8.13-8.01 (m, 2H), 7.99-7.86 (m, 2H), 7.77-7.69 (m, 2H), 7.68-7.61 (m, 1H), 7.60-7.28 (m, 8H), 7.19 (d, J=8.0 Hz, 1H), 7.10 (t, J=3.6 Hz, 1H), 4.93-4.83 (m, 1H), 4.69-4.53 (m, 3H), 4.04-3.89 (m, 2H), 2.03-1.89 (m, 2H).

### Example 233. Synthesis of 5-[[9-ethyl-7-(4-methyl-2-thienyl)carbazol-3-yl]methyl]-N-[6-(2-thienylsulfonylamino)-1,3-benzothiazol-2-yl]-4,6,7,8-tetrahydropyrazolo[1,5-a][1,4]diazepine-2-carboxamide (Compound 154)

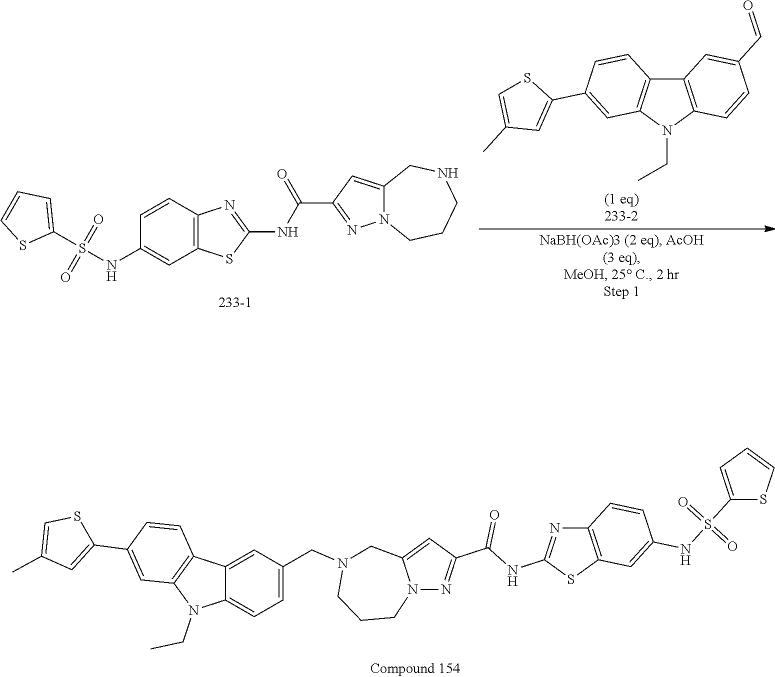

### Step 1: Synthesis of Compound 154

Detailed Synthetic Procedure: To a solution of intermediate 233-1 (30 mg, 63.21 umol, 1 eq) and intermediate 233-2 (22.21 mg, 69.54 umol, 1.1 eq) in MeOH (1 mL) was added NaBH₃CN (11.92 mg, 189.64 umol, 3 eq) and HOAc (3.80 mg, 63.21 umol, 3.62 uL, 1 eq). The mixture was stirred at 25° C. for 2 hr. LCMS (EC5839-72-P1A3) showed SM was consumed completely and 36% of desired mass was detected. The mixture was concentrated under reduced pressure to give a residue. The mixture was purified by prep-HPLC (column: Phenomenex luna C18 150*25 mm*10 um; mobile phase: [water (FA)-ACN];B %: 32%-62%, 10 min) to give a residue. Then residue was concentrated in vacuo and lyophilized to give Compound 154 (21.11 mg, 26.05 umol, 41.21% yield, 96% purity) as an off-white solid and confirmed by LCMS and 1H NMR.

### Mass:

Retention time=0.683 min, (M+H)=778.2

Retention time=0.600 min, (M+H)=778.0

### NMR Data:

1H NMR (400 MHz, DMSO-d6) δ=12.25-12.12 (m, 1H), 10.44 (s, 1H), 8.12 (d, J=7.6 Hz, 1H), 8.03 (br s, 1H), 7.87 (d, J=5.6 Hz, 1H), 7.83 (s, 1H), 7.74 (s, 1H), 7.66 (d, J=8.8 Hz, 1H), 7.57-7.52 (m, 2H), 7.49 (s, 1H), 7.44-7.37 (m, 2H), 7.19 (br d, J=8.8 Hz, 1H), 7.14-7.07 (m, 2H), 6.84 (s, 1H), 4.52-4.44 (m, 4H), 3.93 (br s, 2H), 3.70 (br s, 2H), 3.09 (br s, 2H), 2.28 (s, 3H), 1.90-1.81 (m, 2H), 1.35-1.32 (m, 3H)

### Example 234. Synthesis of [3-[6-[[1-(2,2-difluoro-1,3-benzodioxol-5-yl)cyclopropanecarbonyl]amino]-3-methyl-2-pyridyl]-N-[9-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-9-oxo-nonyl]benzamide] (Compound 155)

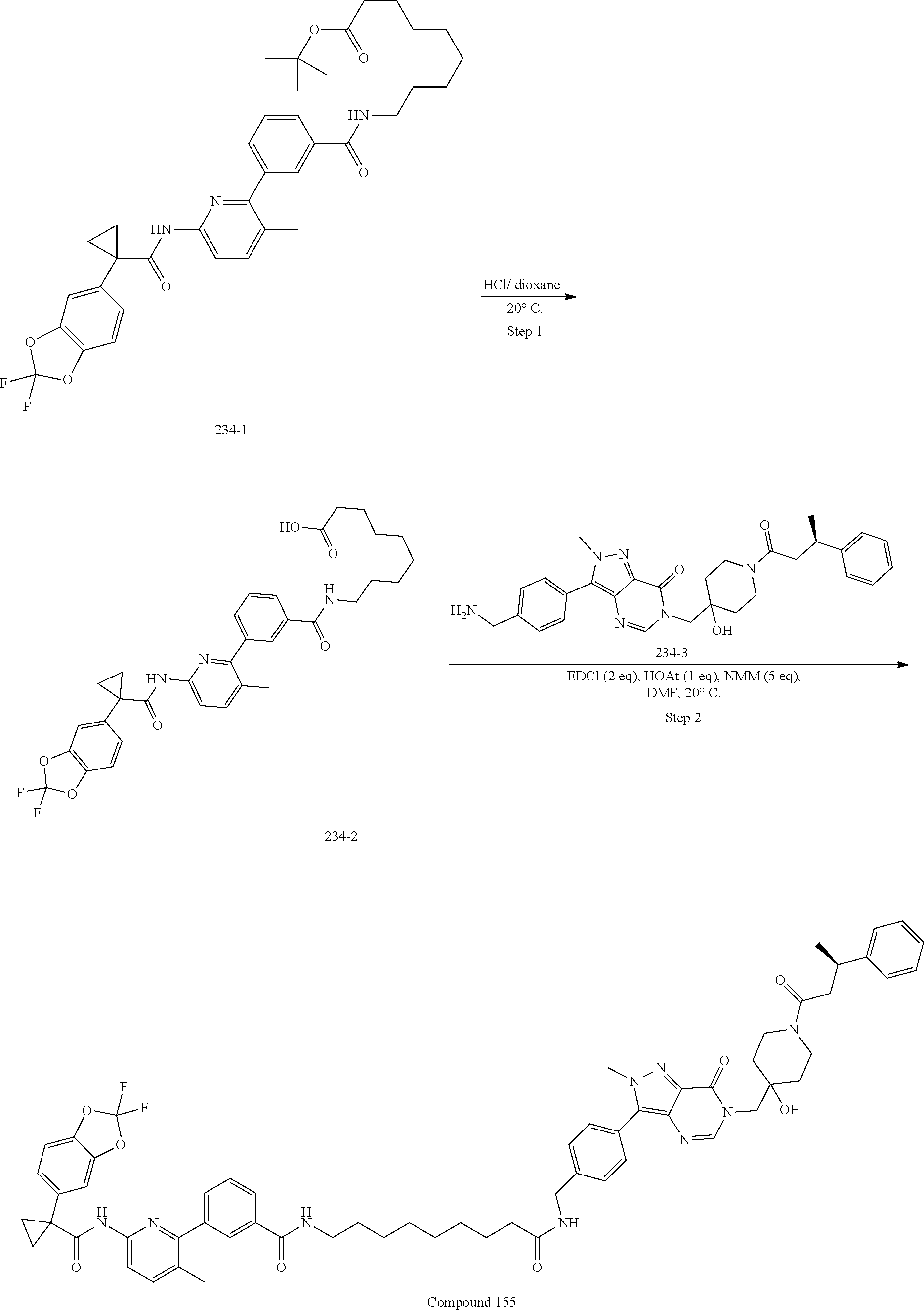 [SMILES: Cc1ccc(NC(=O)C2(c3ccc4c(c3)OC(F)(F)O4)CC2)nc1-c1cccc(C(=O)NCCCCCCCCC(=O)OC(C)(C)C)c1 ; Cc1ccc(NC(=O)C2(c3ccc4c(c3)OC(F)(F)O4)CC2)nc1-c1cccc(C(=O)NCCCCCCCCC(=O)O)c1 ; C[C@H](CC(=O)N1CCC(O)(Cn2cnc3c(-c4ccc(CN)cc4)n(C)nc3c2=O)CC1)c1ccccc1 ; Cc1ccc(NC(=O)C2(c3ccc4c(c3)OC(F)(F)O4)CC2)nc1-c1cccc(C(=O)NCCCCCCCCC(=O)NCc2ccc(-c3c4ncn(CC5(O)CCN(C(=O)C[C@@H](C)c6ccccc6)CC5)c(=O)c4nn3C)cc2)c1]

### Step 1: Synthesis of Intermediate 234-2

Detailed Synthetic Procedure: A mixture of intermediate 234-1 (130 mg, 195.86 umol, 1 eq) in HCl/dioxane (4 M, 1.5 mL) was stirred at 20° C. for 2 hrs. LCMS showed desired molecular weight was detected. The mixture was concentrated to give intermediate 234-2 (115 mg, 189.26 umol, 96.63% yield) as white solid.

### Mass Found:

Retention time: 0.946 min, (M+H)=608.2

### Step 2: Synthesis of Compound 155

Detailed Synthetic Procedure: To a solution of intermediate 234-2 (80 mg, 131.66 umol, 1 eq) and intermediate 234-3 (79.81 mg, 144.82 umol, 1.1 eq, HCl) in DMF (1.5 mL) was added EDCI (50.48 mg, 263.31 umol, 2 eq), HOAt (17.92 mg, 131.66 umol, 18.42 uL, 1 eq) and NMM (66.59 mg, 658.28 umol, 72.38 uL, 5 eq). The mixture was stirred at 20° C. for 2 hrs. LCMS (EW33785-40-P1A2) showed desired molecular weight was detected. The mixture was diluted with water (20 mL) and filtered. The cake was washed with water (10 mL) and collected. The crude was purified by prep-HPLC (column: Phenomenex luna C18 150*25 mm*10 um; mobile phase: [water (FA)-ACN]; B %: 62%-92%, 10 min) and lyophilized to give Compound 155 (55.86 mg, 50.59 umol, 38.42% yield, 100% purity) as white solid which was confirmed by LCMS, 1HNMR, 19FNMR and SFC.

### Mass Found:

Retention time: 0.979 min, (M+H)=1104.4

Retention time: 1.012 min, (M+H)=1104.5

### SFC Found

SFC: Retention time: 1.105 min; AD-3-MeOH+CAN (DEA)-60-3 mL-35T.

### NMR Data:

1H NMR (400 MHz, METHANOL-d4) δ=8.06 (d, J=8.4 Hz, 1H), 8.00-7.91 (m, 1H), 7.83 (td, J=2.4, 6.0 Hz, 1H), 7.80 (s, 1H), 7.72 (d, J=8.4 Hz, 1H), 7.60 (d, J=8.0 Hz, 2H), 7.54-7.47 (m, 4H), 7.37 (d, J=1.6 Hz, 1H), 7.34-7.28 (m, 2H), 7.27-7.24 (m, 2H), 7.23-7.13 (m, 2H), 4.45 (s, 2H), 4.26-4.14 (m, 1H), 4.13-4.06 (m, 3H), 4.05-3.81 (m, 2H), 3.74-3.58 (m, 1H), 3.37-3.33 (m, 2H), 3.28-3.12 (m, 2H), 3.07-2.86 (m, 1H), 2.83-2.68 (m, 1H), 2.64-2.43 (m, 1H), 2.29-2.24 (m, 2H), 2.23 (s, 3H), 1.70-1.55 (m, 7H), 1.41-1.30 (m, 13H), 1.26-1.20 (m, 2H)

### Example 235. Synthesis of [3-[6-[[1-(2,2-difluoro-1,3-benzodioxol-5-yl)cyclopropanecarbonyl]amino]-3-methyl-2-pyridyl]-N-[6-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-6-oxo-hexyl]benzamide] (Compound 156)

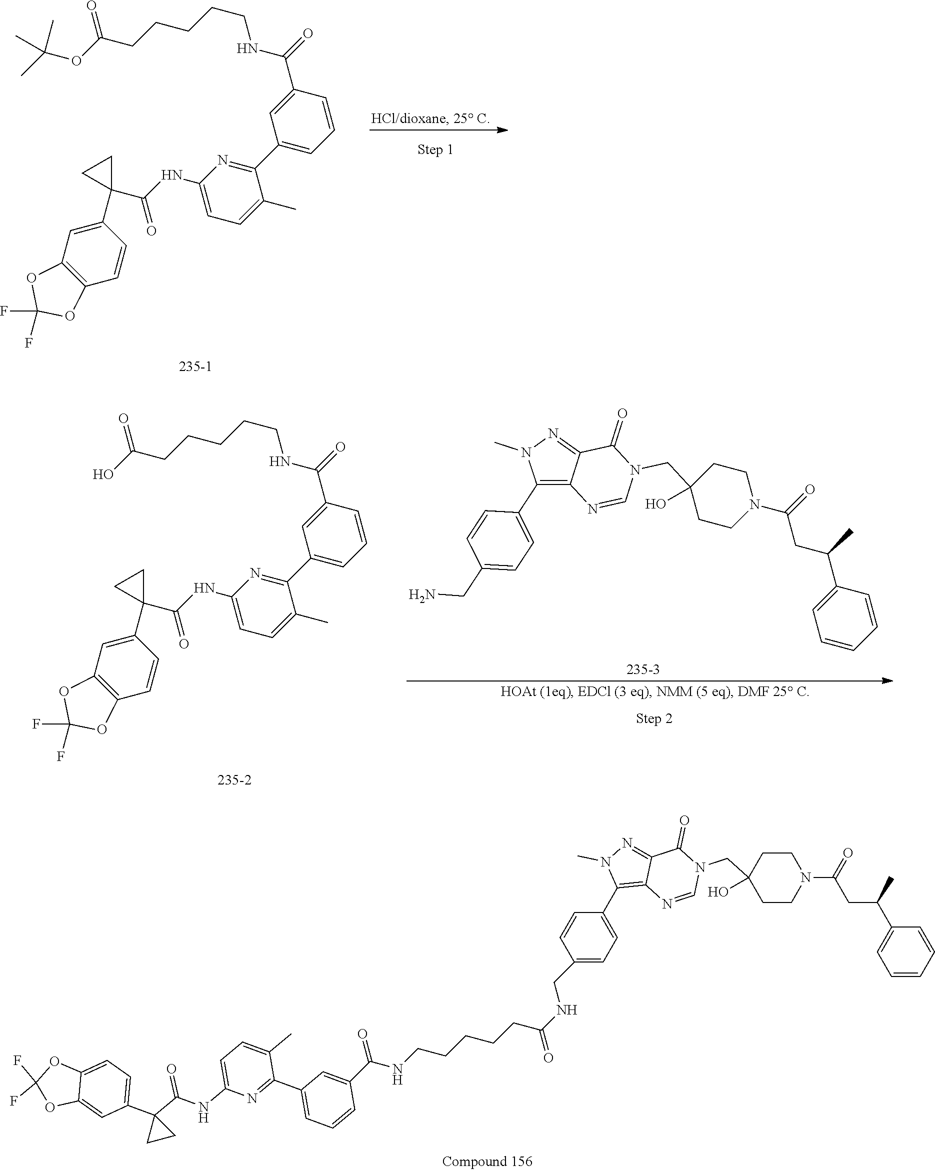 [SMILES: Cc1ccc(NC(=O)C2(c3ccc4c(c3)OC(F)(F)O4)CC2)nc1-c1cccc(C(=O)NCCCCCC(=O)OC(C)(C)C)c1 ; Cc1ccc(NC(=O)C2(c3ccc4c(c3)OC(F)(F)O4)CC2)nc1-c1cccc(C(=O)NCCCCCC(=O)O)c1 ; C[C@H](CC(=O)N1CCC(O)(Cn2cnc3c(-c4ccc(CN)cc4)n(C)nc3c2=O)CC1)c1ccccc1 ; Cc1ccc(NC(=O)C2(c3ccc4c(c3)OC(F)(F)O4)CC2)nc1-c1cccc(C(=O)NCCCCCC(=O)NCc2ccc(-c3c4ncn(CC5(O)CCN(C(=O)C[C@@H](C)c6ccccc6)CC5)c(=O)c4nn3C)cc2)c1]

### Step 1: Synthesis of Intermediate 235-2

Detailed Synthetic Procedure: A solution of intermediate 235-1 (150 mg, 241.29 umol, 1 eq) in HCl/dioxane (1 mL) was stirred at 25° C. for 1 hr. LCMS showed desired molecular weight was detected. The reaction solution was concentrated in vacuum to give intermediate 235-2 (136 mg, 240.47 umol, 99.66% yield) as white oil.

### Mass Found:

Retention time: 0.939 min, (M+H)=566.4

### Step 2: Synthesis of Compound 156

Detailed Synthetic Procedure: To a solution of intermediate 235-2 (136 mg, 240.47 umol, 1 eq) and intermediate 235-3 (139.14 mg, 252.49 umol, 1.05 eq, HCl) in DMF (1 mL) was added EDCI (138.29 mg, 721.40 umol, 3 eq) and HOAt (32.73 mg, 240.47 umol, 33.64 uL, 1 eq) and NN (121.62 mg, 1.20 mmol, 132.19 uL, 5 eq). The mixture was stirred at 25° C. for 2 hrs. LCMS showed desired molecular weight was detected. The residue was diluted with H₂O (20 mL) and extracted with DCM (25 mL*2). The combined organic layers were washed with brine (10 mL), dried over anhydrous Na₂SO₄, filtered and concentrated in vacuum. The crude was purified by prep-HPLC (column: Waters Xbridge 150*25 mm*5 um; mobile phase: [water (ammonia hydroxide v/v)-ACN]; B %: 48%-78%, 8.5 min) to give Compound 1 (85.4 mg, 80.40 umol, 33.44% yield, 100% purity) was obtained as off-white solid which was confirmed by 1HNMR, 19FNMR, LCMS and SFC.

### Mass Found:

Retention time: 0.992 min, (M/2+H)=532.1

Retention time: 1.046 min, (M+H)=1062.6

### SFC Found

SFC: Retention time: 2.365 min; OJ-3-MeOH (DEA)-5-40-3 mL-35T.

### NMR Data:

1H NMR (400 MHz, DMSO-d6) δ=8.94 (s, 1H), 8.47 (m, 1H), 8.44-8.35 (m, 1H), 7.97 (d, 1H), 7.93-7.85 (m, 3H), 7.73 (d, J=8.4 Hz, 1H), 7.65 (d, 2H), 7.55 (s, 2H), 7.53-7.48 (m, 1H), 7.43 (d, 2H), 7.40-7.33 (m, 2H), 7.30-7.23 (m, 4H), 7.16 (m, 1H), 4.86 (d, J=4.4 Hz, 1H), 4.35 (m, 2H), 4.09 (s, 3H), 4.06-3.96 (m, 2H), 3.92 (m, 1H), 3.71-3.60 (m, 1H), 3.30 (s, 3H), 3.26 (m, 2H), 3.19 (m, 2H), 2.96-2.80 (m, 2H), 2.21 (s, 3H), 2.19 (s, 1H), 1.63-1.49 (m, 7H), 1.44-1.26 (m, 6H), 1.21 (d, 3H) 19F NMR (377 MHz, DMSO-d6)

### Example 236. Synthesis of [[(E)-5-(5-(4-(2-(5-oxo-3-phenyl-4-(2-(thiazol-2-yl)hydrazono)-4,5-dihydro-1H-pyrazol-1-yl)thiazol-4-yl)benzamido)pentyl)-N-(6-(thiophene-2-sulfonamido)benzo[d]thiazol-2-yl)-5,6,7,8-tetrahydro-4H-pyrazolo[1,5-a][1,4]diazepine-2-carboxamide]] (Compound 157)

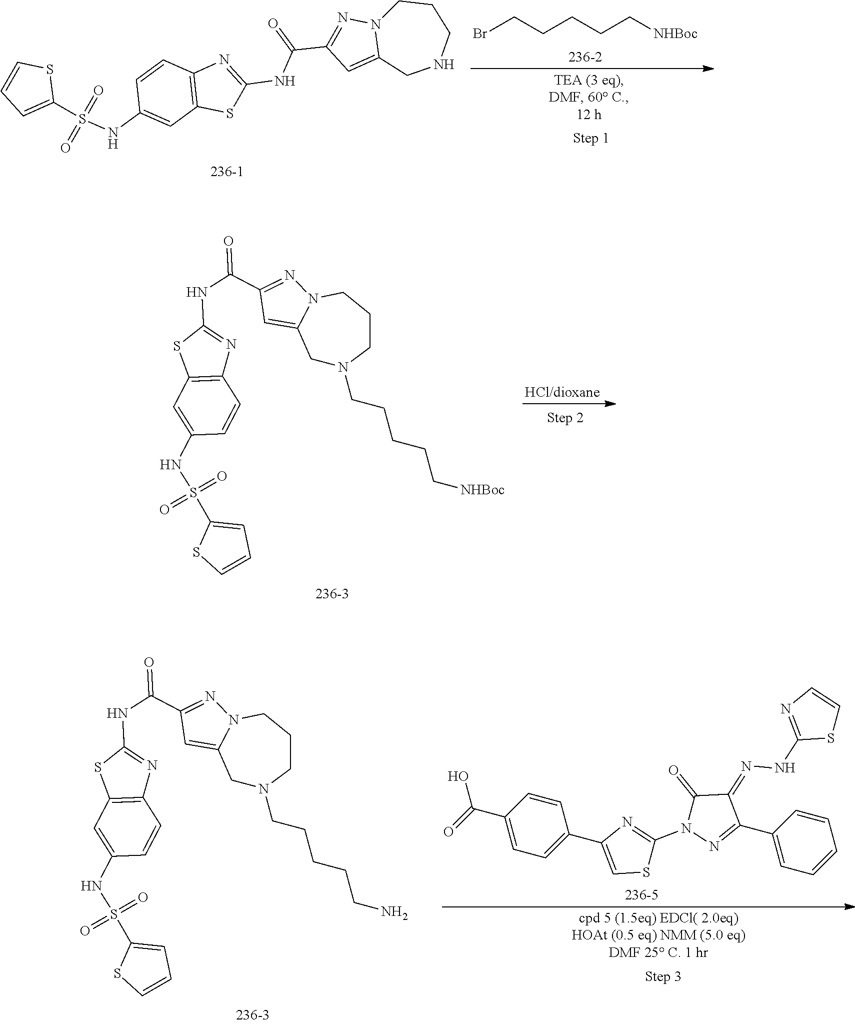

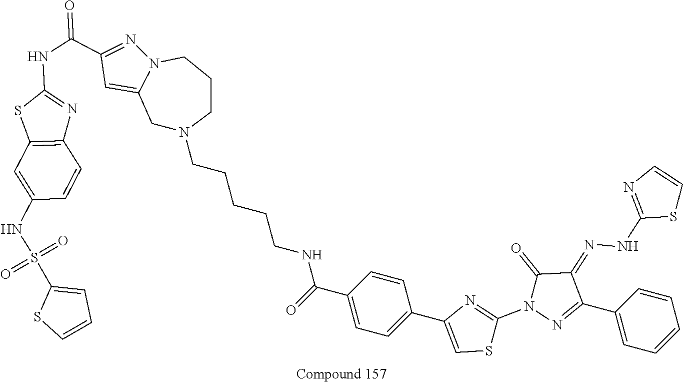 [SMILES: O=C(NCCCCCN1CCCn2nc(C(=O)Nc3nc4ccc(NS(=O)(=O)c5cccs5)cc4s3)cc2C1)c1ccc(-c2csc(N3N=C(c4ccccc4)/C(=N\Nc4nccs4)C3=O)n2)cc1]

### Step 1: Synthesis of Intermediate 236-3

Detailed Synthetic Procedure: To a solution of intermediate 236-1 (0.3 g, 632.14 umol, 1 eq) and intermediate 236-2 (504.78 mg, 1.90 mmol, 3 eq) in DMF (3 mL) was added TEA (191.90 mg, 1.90 mmol, 263.96 uL, 3 eq) at 25° C., then the mixture was stirred at 60° C. for 12 hrs. LCMS showed desired mass was detected. The mixture was quenched with H2O (5 mL) and extracted with EA 15 mL (5 mL*3). The organic layers were dried over Na2SO4, filtered and concentrated under reduced pressure to give a residue. The residue was purified by column chromatography (SiO2, EA:MeOH=10:1 Rf=0.4) and concentrated to give intermediate 236-3 (0.4 g, 606.21 umol, 95.90% yield, N/A purity) as yellow solid and confirmed by LCMS.

### Mass Found

LCMS: Retention time: 0.477 min, (M+H)=660.1

LCMS: Retention time: 0.475 min, (M+H)=660.1

### Step 2: Synthesis of Intermediate 236-4

Detailed Synthetic Procedure: To a solution of intermediate 236-3 (0.1 g, 151.55 umol, 1 eq) in dioxane (1 mL) was added HCl/dioxane (4 M, 2.00 mL), the mixture was stirred at 25° C. for 0.5 hr. LCMS showed 86.78% desired mass was detected. The mixture was concentrated to give a yellow solid intermediate 263-4 (0.08 g, 134.19 umol, 88.54% yield, N/A purity, HCl). The product was taken to the next step directly without purification.

### Mass Found

LCMS: Retention time: 0.270 min, (M+H)=560.2

### Step 3: Synthesis of Compound 157

Detailed Synthetic Procedure: To a solution of intermediate 236-4 (0.07 g, 117.41 umol, 1 eq, HCl) and intermediate 236-5 (55.71 mg, 117.41 umol, 1 eq) in DMF (0.7 mL) was added EDCI (45.02 mg, 234.83 umol, 2 eq), HOAt (7.99 mg, 58.71 umol, 8.21 uL, 0.5 eq) and NMM (59.38 mg, 587.06 umol, 64.54 uL, 5 eq), the mixture was stirred at 25° C. for 1 hr. LCMS showed 66.79% desired mass was detected. The mixture was quenched with water and the precipitated solid was collected by filtration. The obtained solid was purified by Prep-HPLC (column: Phenomenex luna C18 150*25 mm*10 um; mobile phase: [water (TFA)-ACN]; B %: 40%-70%, 10 min) and the eluent was lyophilized to give compound 157 (8 mg, 7.87 umol, 6.70% yield, 100% purity) as an orange solid which was confirmed by LCMS, HNMR, and FNMR.

### Mass Found

LCMS: Retention time: 0.477 min, (M+H)=1016.1

LCMS: Retention time: 0.476 min, (M+H)=1016.0

### NMR Data

1H NMR (400 MHz, DMSO-d6) δ=12.68-12.33 (m, 1H), 10.56-10.38 (m, 1H), 8.59-8.42 (m, 1H), 8.18 (d, J=6.8 Hz, 2H), 8.08 (d, J=8.4 Hz, 2H), 7.97-7.86 (m, 4H), 7.76 (d, J=2.4 Hz, 1H), 7.71 (d, J=3.6 Hz, 1H), 7.67 (d, J=8.4 Hz, 1H), 7.58-7.47 (m, 4H), 7.33 (d, J=3.6 Hz, 1H), 7.26 (s, 1H), 7.22-7.17 (m, 1H), 7.12-7.08 (m, 1H), 4.84-4.64 (m, 2H), 4.59 (d, J=3.6 Hz, 2H), 3.63-3.59 (m, 2H), 3.08-3.03 (m, 2H), 2.43-2.40 (m, 2H), 2.29-2.09 (m, 2H), 1.74 (s, 2H), 1.63-1.56 (m, 2H), 1.40-1.32 (m, 2H).

### Example 237. Synthesis of [3-[6-[[1-(2,2-difluoro-1,3-benzodioxol-5-yl)cyclopropanecarbonyl]amino]-3-methyl-2-pyridyl]-N-[8-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-8-oxo-octyl]benzamide] (Compound 158)

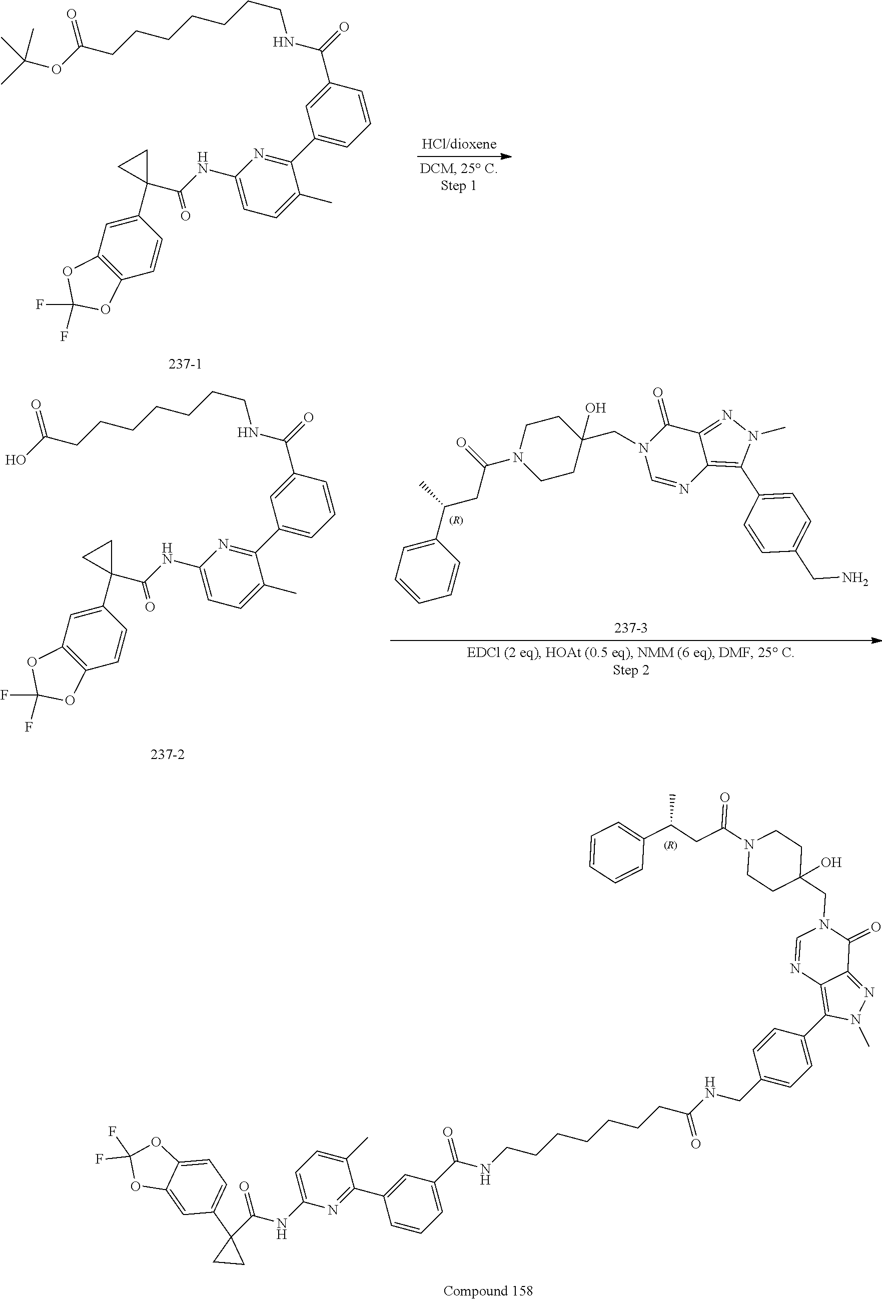

### Step 1: Synthesis of Intermediate 237-2

Detailed Synthetic Procedure: To a solution of intermediate 237-1 (70 mg, 107.74 umol, 1 eq) in DCM (0.5 mL) was added HCl/dioxane (4 M, 1 ml). The mixture was stirred at 25° C. for 2 hrs. LCMS showed desired molecular weight was detected. The mixture was concentrated under vacuum to give intermediate 237-2 (63 mg, 106.13 umol, 98.51% yield) as a yellow solid.

### Mass Found:

Retention time: 0.946 min, (M+H)⁺=594.1

### Step 2: Synthesis of Compound 1

Detailed Synthetic Procedure: To a mixture of intermediate 237-2 (63 mg, 106.13 umol, 1 eq) and intermediate 237-3 (58.49 mg, 106.13 umol, 1 eq, HCl) in DMF (1 mL) was added EDCI (40.69 mg, 212.26 umol, 2 eq), HOAt (7.22 mg, 53.06 umol, 7.42 uL, 0.5 eq) and NN (64.41 mg, 636.77 umol, 70.01 uL, 6 eq). The mixture was stirred at 25° C. for 3 hrs. LCMS showed desired molecular weight was detected. The mixture was poured into water (10 mL) and the formed precipitate was filtered. The obtained solid was further purified by prep-HPLC (column: Phenomenex Synergi C18 150*25 mm*10 um; mobile phase: [water (FA)-ACN]; B %: 61%-81%, 10 min) and the eluent was lyophilized to get Compound 158 (24.43 mg, 21.94 umol, 20.67% yield, 97.9% purity) as a white solid which was confirmed by LCMS, ¹HNMR, ¹⁹FNMR and SFC.

### Mass Found:

Retention time: 0.934 min, (M+H)⁺=1090.4

Retention time: 0.917 min, (M+H)⁺=1090.4

### NMR Data:

1H NMR (400 MHz, DMSO-d6) δ=8.94 (s, 1H), 8.47 (t, J=5.6 Hz, 1H), 8.39 (t, J=6.0 Hz, 1H), 7.98 (d, J=10.0 Hz, 1H), 7.93-7.88 (m, 2H), 7.86 (d, J=7.6 Hz, 1H), 7.73 (d, J=8.8 Hz, 1H), 7.66 (d, J=8.0 Hz, 2H), 7.59-7.54 (m, 2H), 7.52-7.47 (m, 1H), 7.43 (d, J=8.4 Hz, 2H), 7.40-7.33 (m, 2H), 7.30-7.23 (m, 4H), 7.20-7.13 (m, 1H), 4.87 (d, J=4.8 Hz, 1H), 4.35 (d, J=6.0 Hz, 2H), 4.10 (s, 3H), 4.06-3.87 (m, 3H), 3.75-3.59 (m, 1H), 3.28-3.13 (m, 4H), 2.93-2.83 (m, 1H), 2.62-2.54 (m, 2H), 2.21 (s, 3H), 2.17 (t, J=7.2 Hz, 2H), 1.61-1.44 (m, 7H), 1.37 (br d, J=12.8 Hz, 2H), 1.31 (br s, 7H), 1.21 (d, J=6.8 Hz, 3H), 1.19-1.15 (m, 2H).

SFC: Rt=0.504 min; OJ-3-MeOH+ACN (DEA)-40-3ML-35T

### Example 238. Synthesis of [3-[6-[[1-(2,2-difluoro-1,3-benzodioxol-5-yl)cyclopropanecarbonyl]amino]-3-methyl-2-pyridyl]-N-[7-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-7-oxo-heptyl]benzamide] (Compound 159)

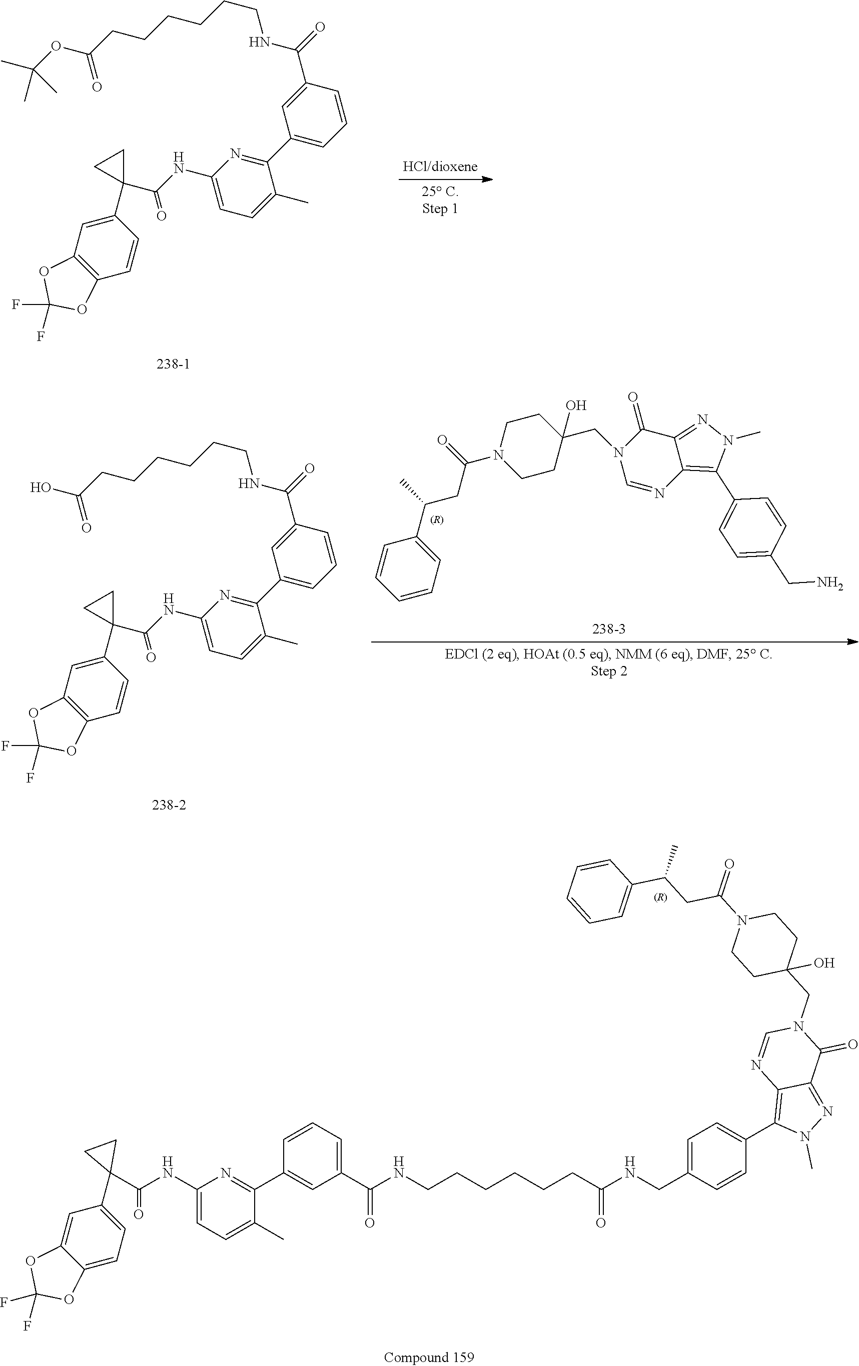

### Step 1: Synthesis of Intermediate 238-2

Detailed Synthetic Procedure: A mixture of intermediate 238-1 (80 mg, 125.85 umol, 1 eq) in HCl/dioxane (1 Ml, 4M) was stirred at 25° C. for 2 hrs. LCMS showed desired molecular weight was detected. The reaction mixture was concentrated under reduce pressure to get intermediate 238-2 (72.5 mg, 125.09 umol, 99.40% yield) as red solid.

### Mass:

Retention time: 0.944 min, (M+H)=580.1

### Step 2: Synthesis of Compound 159

Detailed Synthetic Procedure: To a solution of intermediate 238-2 (72 mg, 124.23 umol, 1 eq) in DMF (1 mL) was added intermediate 238-3 (68.46 mg, 124.23 umol, 1 eq, HCl), EDCI (47.63 mg, 248.45 umol, 2 eq), HOAt (8.45 mg, 62.11 umol, 8.69 uL, 0.5 eq) and NMM (75.39 mg, 745.35 umol, 81.95 uL, 6 eq). The mixture was stirred at 25° C. for 14 hrs. LCMS showed desired molecular weight was detected. The reaction mixture was poured into water (10 mL) and filtered to get the filter cake. The filter cake was purified by reversed-phase HPLC (0.1% FA condition) and the eluent was lyophilized to give Compound 159 (30.11 mg, 26.25 umol, 21.13% yield, 93.83% purity) as white solid which was confirmed by LCMS, chiral SFC and ¹HNMR.

### Mass Found:

Retention time: 0.920 min, (M+H)=1076.3

Retention time: 0.909 min, (M+H)=1076.4

### NMR Data:

1H NMR (400 MHz, DMSO-d6) δ=8.93 (s, 1H), 8.54-8.27 (m, 2H), 7.99-7.82 (m, 4H), 7.72 (d, J=8.4 Hz, 1H), 7.66 (d, J=8.4 Hz, 2H), 7.60-7.53 (m, 2H), 7.53-7.47 (m, 1H), 7.43 (d, J=8.0 Hz, 2H), 7.40-7.31 (m, 2H), 7.30-7.22 (m, 4H), 7.19-7.12 (m, 1H), 4.86 (d, J=4.8 Hz, 1H), 4.35 (d, J=6.0 Hz, 2H), 4.09 (s, 3H), 4.06-3.85 (m, 3H), 3.71-3.58 (m, 1H), 3.27-3.12 (m, 4H), 2.94-2.80 (m, 1H), 2.58 (d, J=7.2 Hz, 2H), 2.25-2.13 (m, 5H), 1.61-1.46 (m, 7H), 1.42-1.28 (m, 7H), 1.24-1.18 (m, 3H), 1.18-1.14 (m, 2H)

SFC: Rt=0.506 min; OJ-3-MeOH+ACN (DEA)-40-3ML-35T

### Example 239. Synthesis of [3-[6-[[1-(2,2-difluoro-1,3-benzodioxol-5-yl)cyclopropanecarbonyl]amino]-3-methyl-2-pyridyl]-N-[5-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-5-oxo-pentyl]benzamide] (Compound 160)

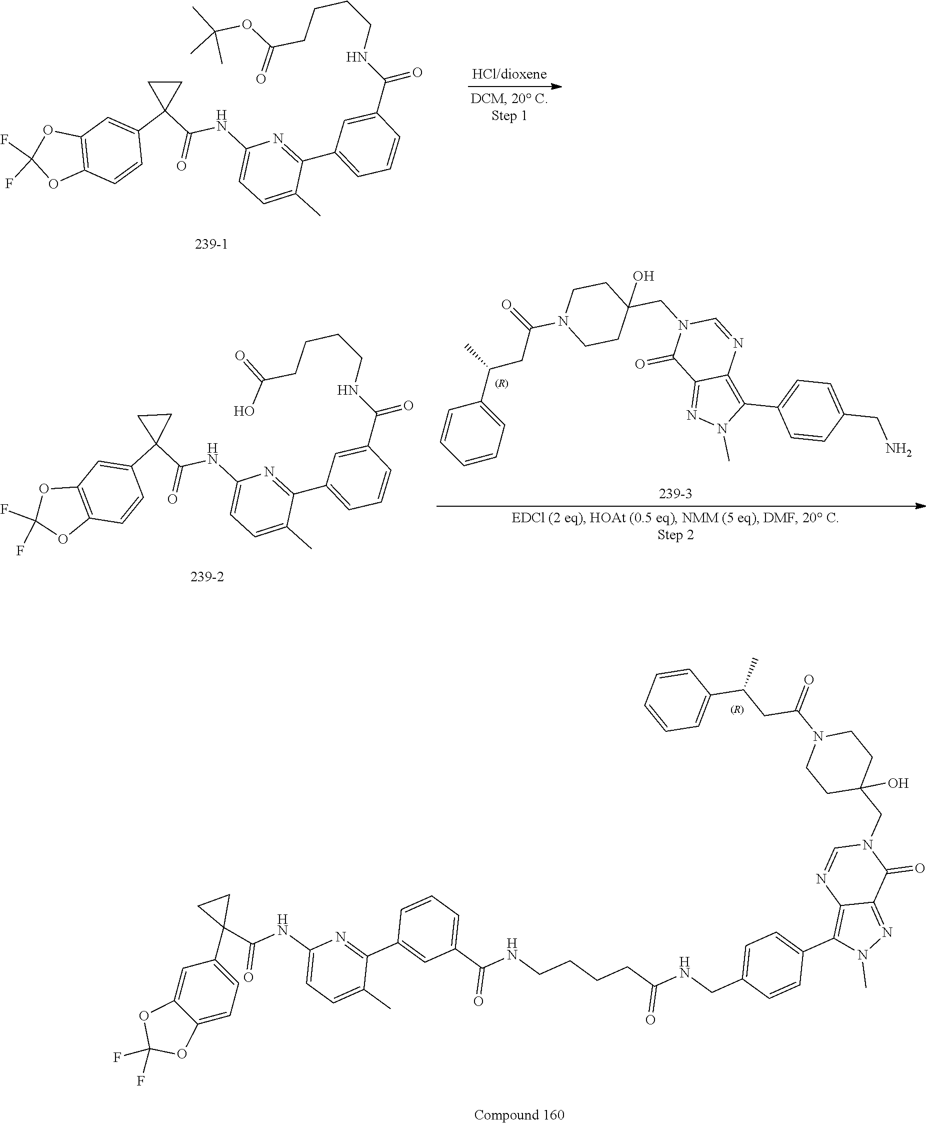

### Step 1: Synthesis of Intermediate 239-2

Detailed Synthetic Procedure: To a solution of intermediate 239-1 (80 mg, 131.66 umol, 1 eq) in DCM (1 mL) was added HCl/dioxane (4 M, 1 mL). The mixture was stirred at 20° C. for 1 hr. LCMS showed desired molecular weight was detected. The reaction mixture was concentrated under reduced pressure to give intermediate 239-2 (70 mg, crude) as yellow oil.

### Mass:

Retention time: 0.948 min, (M+H)=552.3

### Step 2: Synthesis of Compound 160

Detailed Synthetic Procedure: To a solution of intermediate 239-2 (70 mg, 126.92 umol, 0.82 eq) in DMF (3 mL) was added intermediate 239-3 (102.32 mg, 185.67 umol, 1.2 eq, HCl), NN (78.25 mg, 773.62 umol, 85.06 uL, 5 eq), HOAt (10.53 mg, 77.36 umol, 10.82 uL, 0.5 eq) and EDCI (59.32 mg, 309.45 umol, 2 eq). The mixture was stirred at 20° C. for 12 hrs. LCMS showed desired molecular weight was detected. The reaction mixture was quenched by H₂O (50 mL) and extracted with EtOAc (50 mL*2). The combined organic layers were washed with brine (90 mL) and concentrated under reduced pressure. The residue was purified by prep-HPLC (column: Phenomenex luna C18 150*25 mm*10 um; mobile phase: [water (FA)-ACN]; B %: 55%-85%, 10 min) and the eluent was lyophilized to give Compound 160 (66.66 mg, 63.02 umol, 40.73% yield, 99.097% purity) as white solid which was confirmed by LCMS, 1HNMR, 19FNMR and chiral SFC.

### Mass Found:

Retention time: 0.912 min, (M+H)=1048.6

Retention time: 0.920 min, (M+H)=1048.6

### NMR Data:

1H NMR (400 MHz, DMSO-d6) δ=9.01-8.91 (m, 1H), 8.56-8.48 (m, 1H), 8.45-8.40 (m, 1H), 8.02-7.95 (m, 1H), 7.93-7.85 (m, 3H), 7.75-7.69 (m, 1H), 7.67-7.62 (m, 2H), 7.59-7.54 (m, 2H), 7.53-7.48 (m, 1H), 7.47-7.42 (m, 2H), 7.40-7.33 (m, 2H), 7.31-7.23 (m, 4H), 7.21-7.12 (m, 1H), 4.97-4.79 (m, 1H), 4.41-4.31 (m, 2H), 4.14-3.90 (m, 6H), 3.72-3.59 (m, 1H), 3.30-3.26 (m, 2H), 3.23-3.09 (m, 2H), 2.96-2.81 (m, 1H), 2.63-2.56 (m, 2H), 2.25-2.17 (m, 5H), 1.66-1.45 (m, 7H), 1.43-1.28 (m, 3H), 1.28-1.12 (m, 6H)

SFC: Rt=0.507 min; OJ-3-MeOH+ACN (DEA)-40-3ML-35T

### Example 240. Synthesis of [3-[6-[[1-(2,2-difluoro-1,3-benzodioxol-5-yl)cyclopropanecarbonyl]amino]-3-methyl-2-pyridyl]-N-[4-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-4-oxo-butyl]benzamide] (Compound 161)

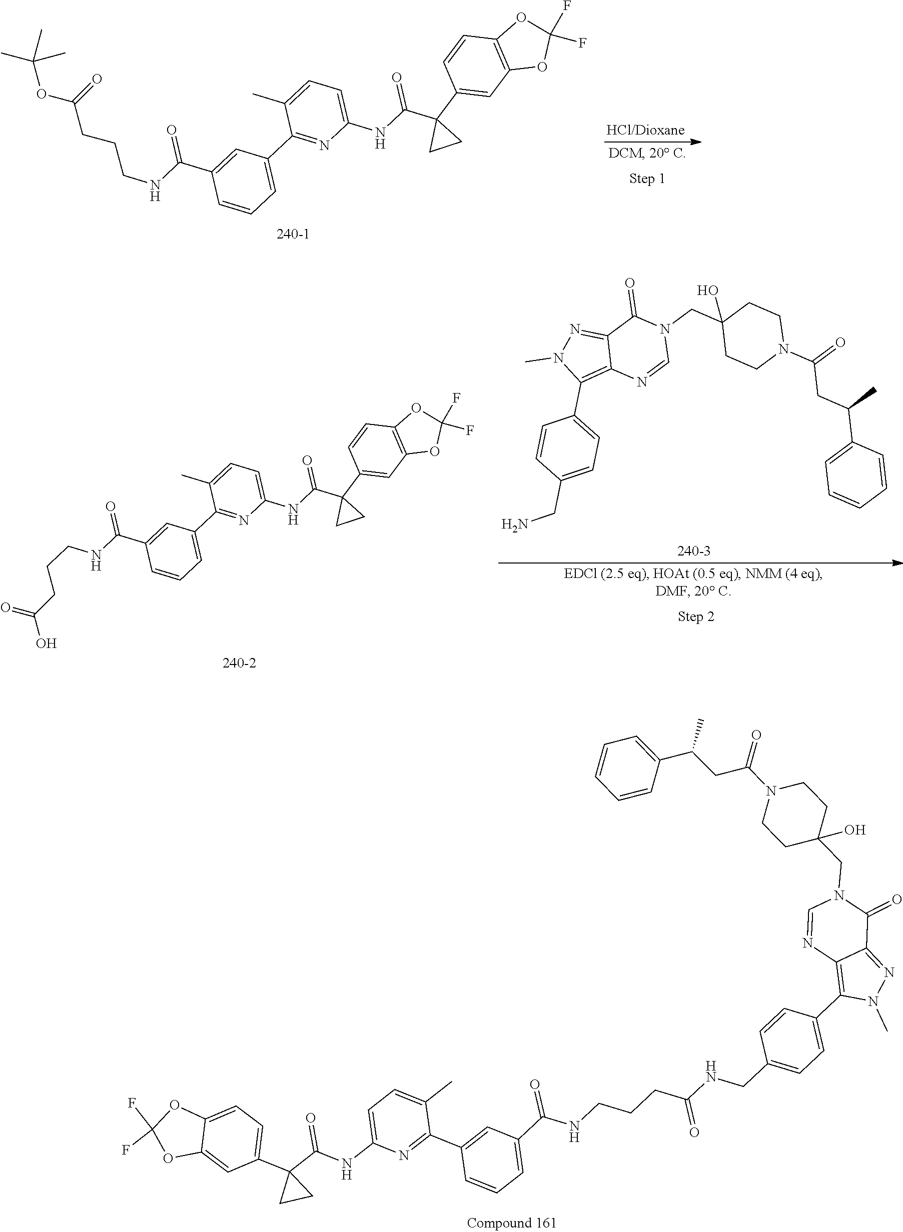 [SMILES: Cc1ccc(NC(=O)C2(c3ccc4c(c3)OC(F)(F)O4)CC2)nc1-c1cccc(C(=O)NCCCC(=O)OC(C)(C)C)c1 ; Cc1ccc(NC(=O)C2(c3ccc4c(c3)OC(F)(F)O4)CC2)nc1-c1cccc(C(=O)NCCCC(=O)O)c1 ; C[C@H](CC(=O)N1CCC(O)(Cn2cnc3c(-c4ccc(CN)cc4)n(C)nc3c2=O)CC1)c1ccccc1 ; Cc1ccc(NC(=O)C2(c3ccc4c(c3)OC(F)(F)O4)CC2)nc1-c1cccc(C(=O)NCCCC(=O)NCc2ccc(-c3c4ncn(CC5(O)CCN(C(=O)C[C@@H](C)c6ccccc6)CC5)c(=O)c4nn3C)cc2)c1]

### Step 1: Synthesis of Intermediate 240-2

Detailed Synthetic Procedure: To a mixture of intermediate 240-1 (70 mg, 117.92 umol, 1 eq) in DCM (1 mL) was added HCl/dioxane (4 M, 1 mL). The mixture was stirred at 20° C. for 1 hr. LCMS showed desired molecular weight was detected. The reaction mixture was concentrated to give intermediate 240-2 (65 mg, crude) as white solid.

### Mass Found:

Retention time: 0.885 min, (M+H)=538.1

### Step 2: Synthesis of Compound 1

Detailed Synthetic Procedure: To a mixture of intermediate 240-2 (65 mg, 120.93 umol, 1 eq), intermediate 240-3 (66.64 mg, 120.93 umol, 1 eq, HCl) and NMM (48.93 mg, 483.72 umol, 53.18 uL, 4 eq) in DMF (1 mL) was added HOAt (8.23 mg, 60.47 umol, 8.46 uL, 0.5 eq) and EDCI (57.96 mg, 302.33 umol, 2.5 eq). The reaction mixture was stirred at 20° C. for 1 hr. LCMS showed desired molecular weight was detected. The reaction mixture was diluted with MeOH (2 mL) and purified by prep-HPLC (column: Phenomenex luna C18 150*25 mm*10 um; mobile phase: [water (FA)-ACN]; B %: 48%-78%, 10 min) and the eluent was lyophilizated to give Compound 1 (20.66 mg, 19.86 umol, 16.42% yield, 99.4% purity) as white solid which was confirmed by LCMS, SFC, HNMR, and FNMR.

### Mass Found:

Retention time: 1.003 min, (M12+H)=518.0

Retention time: 0.910 min, (M+H)=1034.6

### NMR Data:

¹H NMR (400 MHz, DMSO-d6) δ=8.94 (s, 1H), 8.53 (t, J=5.6 Hz, 1H), 8.48-8.41 (m, 1H), 7.97 (d, J=10.0 Hz, 1H), 7.93-7.86 (m, 3H), 7.72 (d, J=8.4 Hz, 1H), 7.68-7.62 (m, J=8.4 Hz, 2H), 7.59-7.48 (m, 3H), 7.44 (d, J=8.4 Hz, 2H), 7.39-7.35 (m, 1H), 7.35-7.31 (m, 1H), 7.29-7.21 (m, 4H), 7.19-7.12 (m, 1H), 4.99-4.78 (m, 1H), 4.35 (d, J=6.0 Hz, 2H), 4.09 (s, 3H), 4.06-3.96 (m, 2H), 3.95-3.88 (m, 1H), 3.71-3.60 (m, 1H), 3.29-3.26 (m, 2H), 3.24-3.12 (m, 2H), 2.93-2.81 (m, 1H), 2.58 (d, J=7.6 Hz, 2H), 2.29-2.22 (m, 2H), 2.21 (s, 3H), 1.81 (quin, J=7.2 Hz, 2H), 1.53-1.47 (m, 2H), 1.45-1.23 (m, 4H), 1.20 (d, J=6.8 Hz, 3H), 1.18-1.13 (m, 2H).

SFC Data: Rt=0.523 min; method details: column: Chiralcel OJ-3 50×4.6 mm I.D., 3 um; mobile phase: phase A for CO₂, and phase B for MeOH+ACN (0.05% DEA); gradient elution: 40% MeOH+ACN (0.05% DEA) in CO₂; flow rate: 3 mL/min; detector: PDA; column temp: 35° C.; back pressure: 100 Bar.

### Example 241. Synthesis of [3-[6-[[1-(2,2-difluoro-1,3-benzodioxol-5-yl)cyclopropanecarbonyl]amino]-3-methyl-2-pyridyl]-N-[3-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-3-oxo-propyl]benzamide] (Compound 162)

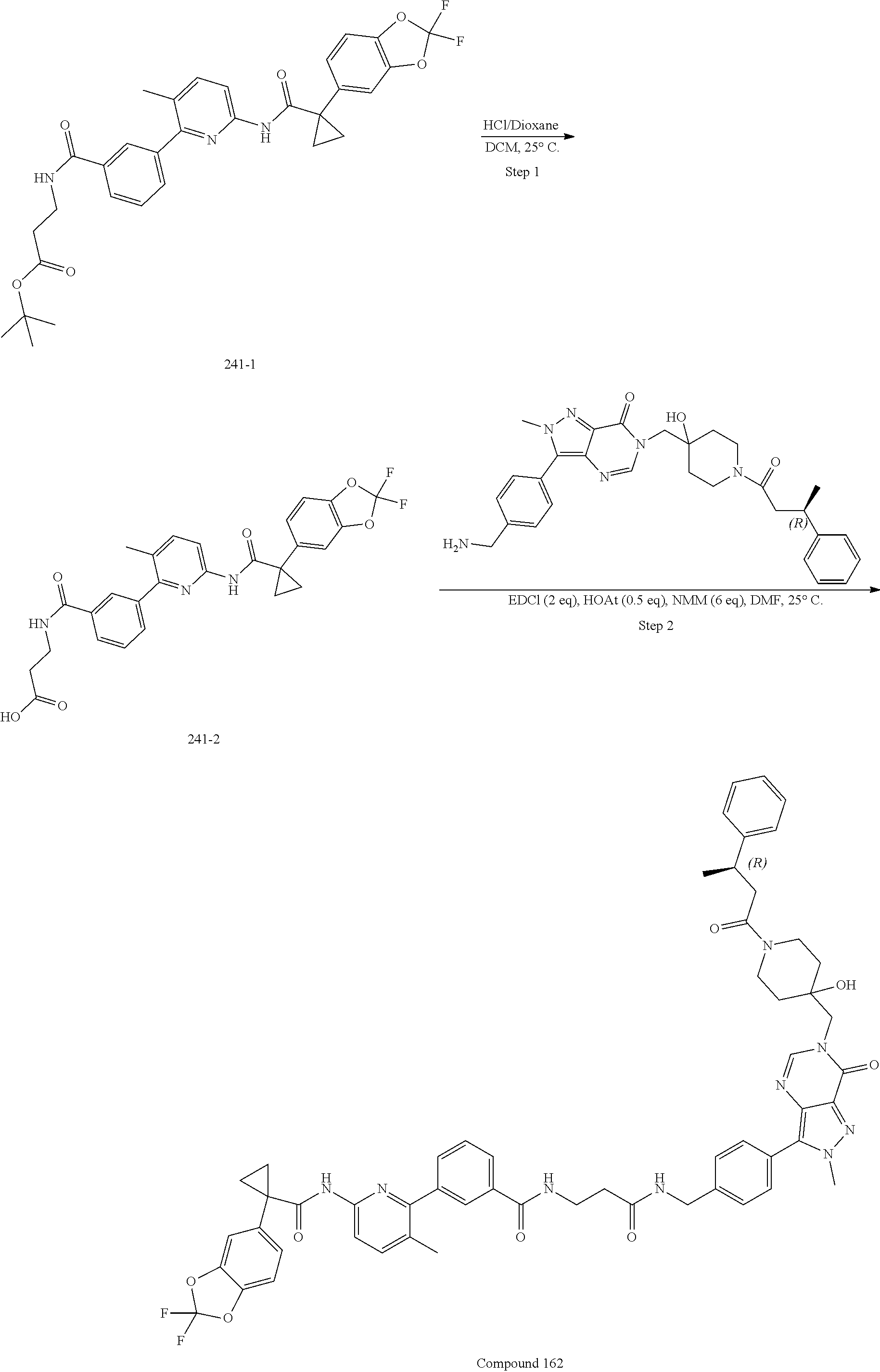

### Step 1: Synthesis of Intermediate 241-2

Detailed Synthetic Procedure: To a solution of intermediate 241-1 (100 mg, 172.54 umol, 1 eq) in DCM (0.2 mL) was added HCl/dioxane (4 M, 1 mL). The reaction was stirred at 25° C. for 2 hrs. LCMS showed desired molecular weight was detected. The reaction mixture was concentrated in vacuum to give intermediate 241-2 (90 mg, 171.93 umol, 99.65% yield) as white solid.

### Mass:

Retention time: 0.884 min, (M+H)=524.0

### Step 2: Synthesis of Compound 1

Detailed Synthetic Procedure: To a solution of intermediate 241-2 (90 mg, 171.93 umol, 1 eq) and intermediate 241-3 (94.74 mg, 171.93 umol, 1 eq, HCl) in DMF (1 mL) was added EDCI (65.92 mg, 343.85 umol, 2 eq), HOAt (11.70 mg, 85.96 umol, 12.03 uL, 0.5 eq) and NN (104.34 mg, 1.03 mmol, 113.41 uL, 6 eq). The reaction mixture was stirred at 25° C. for 12 hrs. LCMS showed desired molecular weight was detected. The reaction mixture was poured into water (10 mL) and solid precipitated. The mixture was filtered and the filter cake was collected and purified by prep-HPLC (column: Phenomenex Synergi C18 150*25 mm*10 um; mobile phase: [water (FA)-ACN]; B %: 52%-79%, 9 min) and lyophilized to give Compound 162 (48.32 mg, 45.48 umol, 26.46% yield, 96.02% purity) as off-white solid which was confirmed by LCMS, HNMR and SFC.

### Mass Found:

Retention time: 0.932 min, (M/2+H)=510.7

Retention time: 0.909 min, (M+H)=1020.6

### NMR Data:

¹H NMR (400 MHz, DMSO-d6) δ=8.92 (s, 1H), 8.61 (br t, J=5.2 Hz, 1H), 8.52 (t, J=6.0 Hz, 1H), 7.97 (d, J=10.0 Hz, 1H), 7.92-7.85 (m, 3H), 7.68 (d, J=8.4 Hz, 1H), 7.60-7.49 (m, 5H), 7.42 (d, J=8.4 Hz, 2H), 7.38-7.23 (m, 6H), 7.18-7.13 (m, 1H), 4.87 (d, J=3.6 Hz, 1H), 4.37 (d, J=5.6 Hz, 2H), 4.06 (s, 3H), 4.04-3.87 (m, 3H), 3.71-3.59 (m, 1H), 3.55-3.49 (m, 2H), 3.24-3.10 (m, 2H), 2.88 (s, 1H), 2.65-2.51 (m, 4H), 2.17 (s, 3H), 1.56-1.26 (m, 6H), 1.20 (d, J=6.8 Hz, 3H), 1.17-1.14 (m, 2H)

SFC Data: Rt=0.499 min; method details: column: Chiralcel OJ-3 50×4.6 mm I.D., 3 um; mobile phase: phase A for CO₂, and phase B for MeOH+ACN (0.05% DEA); gradient elution: 40% MeOH+ACN (0.05% DEA) in CO₂; flow rate: 3 mL/min; detector: PDA; column temp: 35° C.; back pressure: 100 Bar

### Example 242. Synthesis of [3-[6-[[1-(2,2-difluoro-1,3-benzodioxol-5-yl)cyclopropanecarbonyl]amino]-3-methyl-2-pyridyl]-N-[2-[2-[2-[3-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-3-oxo-propoxy]ethoxy]ethoxy]ethyl]benzamide] (Compound 163)

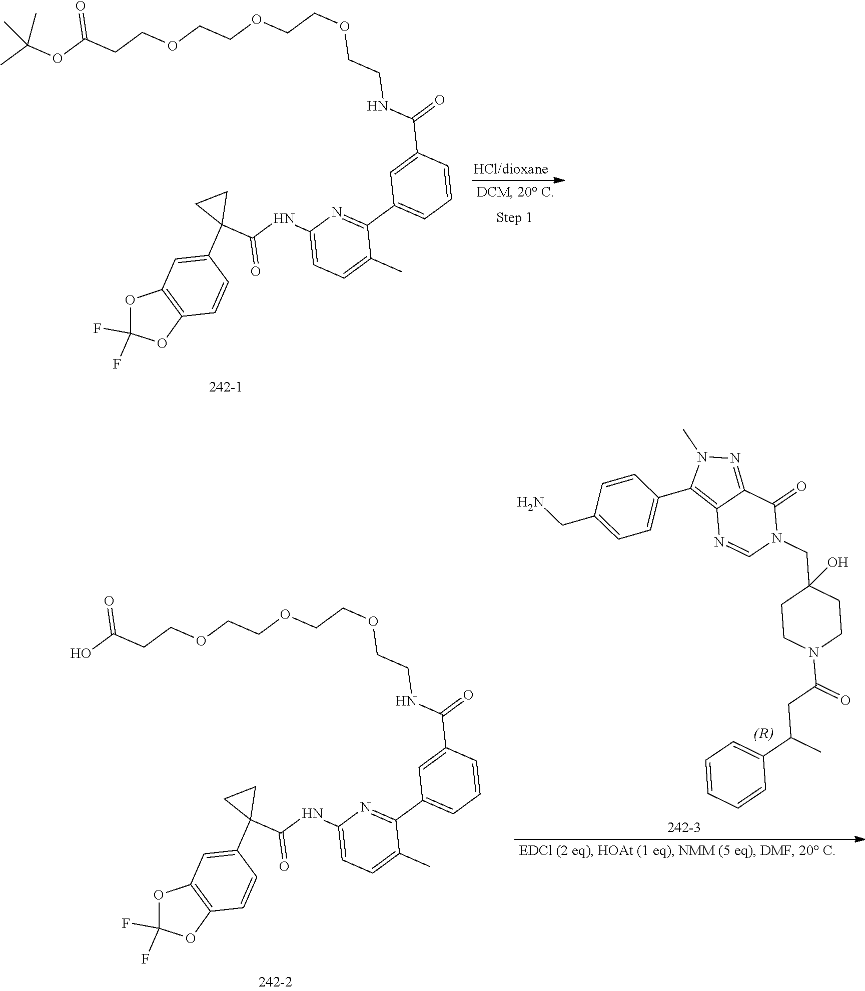

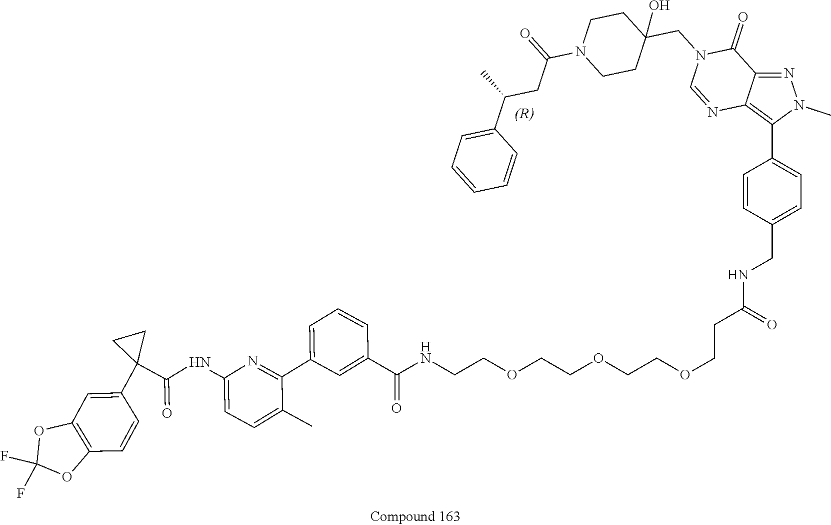

### Step 1: Synthesis of Intermediate 242-2

Detailed Synthetic Procedure: To a solution of intermediate 242-1 (90 mg, 126.45 umol, 1 eq) in DCM (0.5 mL) was added HCl/dioxane (4 M, 1.58 mL). The mixture was stirred at 20° C. for 1 h. LCMS showed desired molecular weight was detected. The reaction mixture was concentrated under reduced pressure to give intermediate 242-2 (87 mg, crude) as white solid.

### Mass:

Retention time: 0.856 min, (M+H)=656.2

### Step 2: Synthesis of Compound 163

Detailed Synthetic Procedure: To a solution of intermediate 242-2 (87 mg, 125.70 umol, 1 eq), intermediate 242-3 (76.20 mg, 138.27 umol, 1.1 eq, HCl) in DMF (1.5 mL) was added EDCI (48.20 mg, 251.41 umol, 2 eq), HOAt (17.11 mg, 125.70 umol, 17.58 uL, 1 eq) and NMM (63.57 mg, 628.52 umol, 69.10 uL, 5 eq). The mixture was stirred at 20° C. for 16 hrs. LC-MS showed desired compound was detected. The reaction mixture was concentrated under reduced pressure. The residue was purified by prep-HPLC (column: Phenomenex luna C18 150*40 mm*15 um; mobile phase: [water (FA)-ACN]; B %: 45%-75%, 10 min) and lyophilized to give Compound 163 (43.99 mg, 37.80 umol, 30.07% yield, 99% purity) as white solid which was confirmed by LCMS, 1HNMR, and SFC.

### Mass Found:

Retention time: 0.885 min

Retention time: 0.880 min

### NMR Data:

1H NMR (400 MHz, DMSO-d6) δ=8.96 (s, 1H), 8.55 (br t, J=5.4 Hz, 1H), 8.46 (br t, J=5.8 Hz, 1H), 8.41 (br s, 1H), 8.02-7.82 (m, 4H), 7.73 (d, J=8.4 Hz, 1H), 7.66 (d, J=8.2 Hz, 1H), 7.60-7.48 (m, 3H), 7.44 (d, J=8.2 Hz, 2H), 7.40-7.31 (m, 2H), 7.29-7.22 (m, 4H), 7.19-7.12 (m, 1H), 4.88 (br s, 1H), 4.37 (br d, J=5.8 Hz, 2H), 4.16-3.85 (m, 6H), 3.73-3.60 (m, 3H), 3.55-3.45 (m, 10H), 3.44-3.38 (m, 3H), 3.22-3.12 (m, 2H), 2.96-2.81 (m, 1H), 2.63-2.56 (m, 1H), 2.41 (br t, J=6.4 Hz, 2H), 2.21 (s, 3H), 1.57-1.46 (m, 2H), 1.44-1.28 (m, 3H), 1.26-1.12 (m, 6H)

### SFC:

Rt=1.026 min; method details: column: Chiralcel OD-3 50×4.6 mm I.D., 3 um; mobile phase: phase A for CO₂, and phase B for MeOH+ACN (0.05% DEA); gradient elution: 60% MeOH+ACN (0.05% DEA) in CO₂; flow rate: 3 mL/min; detector: PDA; column temp: 35° C.; back pressure: 100 Bar

### Example 243. Synthesis of [3-[6-[[1-(2,2-difluoro-1,3-benzodioxol-5-yl)cyclopropanecarbonyl]amino]-3-methyl-2-pyridyl]-N-[2-[2-[3-[[4-[6-[[4-hydroxy-1-[(3R)-3-phenylbutanoyl]-4-piperidyl]methyl]-2-methyl-7-oxo-pyrazolo[4,3-d]pyrimidin-3-yl]phenyl]methylamino]-3-oxo-propoxy]ethoxy]ethyl]benzamide] (Compound 164)

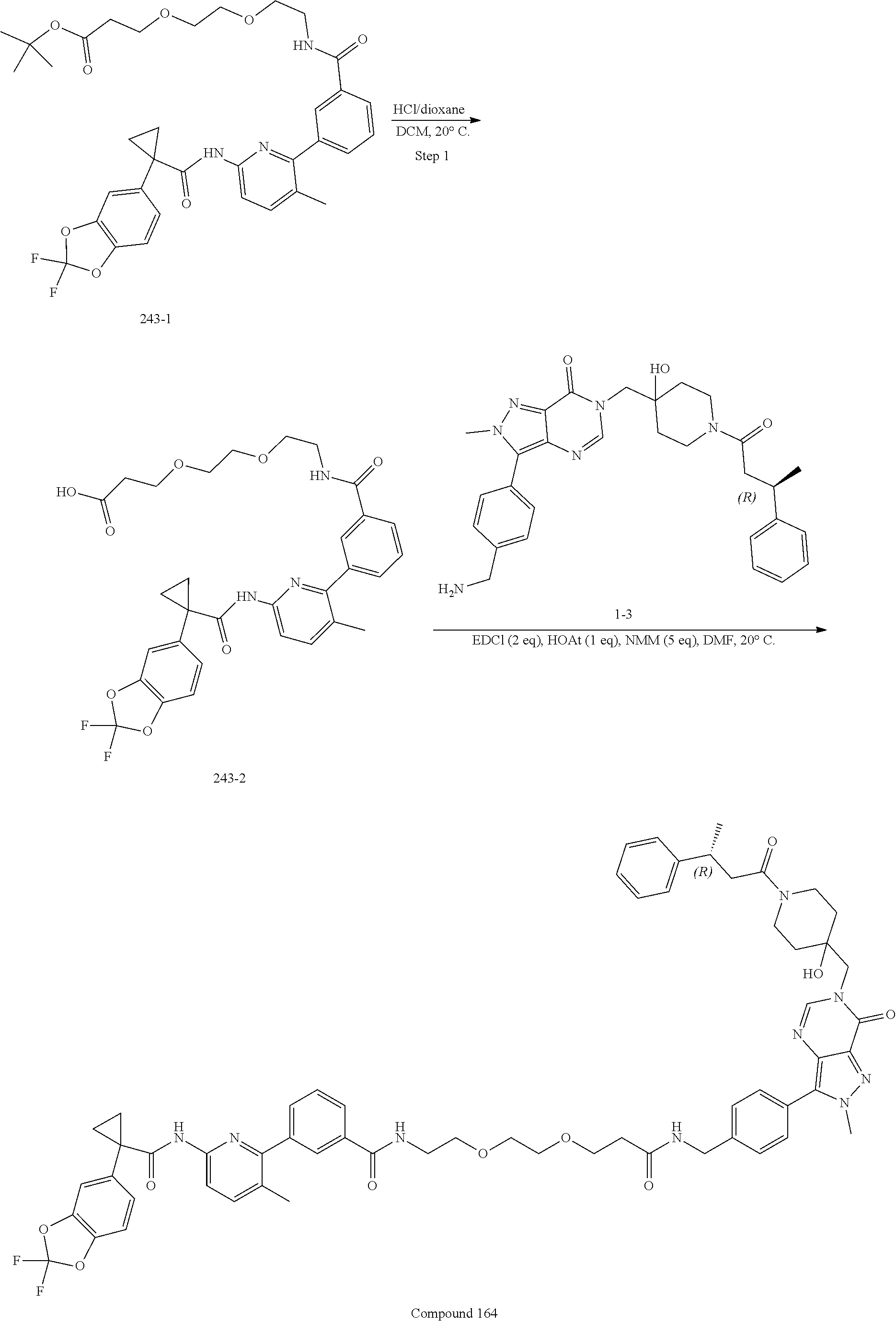

### Step 1: Synthesis of Intermediate 243-2

Detailed Synthetic Procedure: To a solution of intermediate 243-1 (70 mg, 104.84 umol, 1 eq) in DCM (0.5 mL) was added HCl/dioxane (4 M, 1.57 mL). The mixture was stirred at 20° C. for 1 hr. LCMS showed desired molecular weight was detected. The reaction mixture was concentrated to give intermediate 243-2 (68 mg, crude) as white solid.

### Mass Found:

Retention time: 0.736 min, (M+H)=612.2

### Step 2: Synthesis of Compound 164

Detailed Synthetic Procedure: To a solution of intermediate 243-2 (68 mg, 111.19 umol, 1 eq) and intermediate 243-3 (67.40 mg, 122.30 umol, 1.1 eq, HCl) in DMF (1 mL) was added EDCI (42.63 mg, 222.37 umol, 2 eq), HOAt (15.13 mg, 111.19 umol, 15.55 uL, 1 eq) and NN (56.23 mg, 555.93 umol, 61.12 uL, 5 eq). The mixture was stirred at 20° C. for 16 hrs. LCMS showed desired molecular weight was detected. The reaction mixture was diluted with water and purified by prep-HPLC directly (column: Phenomenex luna C18 150*40 mm*15 um; mobile phase: [water (FA)-ACN]; B %: 43%-73%, 10 min) and lyophilized to give Compound 164 (30.16 mg, 27.22 umol, 24.48% yield, 100% purity) as a white solid which was confirmed by LCMS, 1HNMR, 19FNMR and SFC.

### Mass Found:

Retention time: 0.882 min, (M+H)=1108.8

Retention time: 0.880 min, (M+H)=1108.5

### NMR Data:

1H NMR (400 MHz, DMSO-d6) δ=8.96 (s, 1H), 8.55 (t, J=5.4 Hz, 1H), 8.45 (t, J=6.0 Hz, 1H), 8.24 (s, 1H), 7.97 (d, J=10.4 Hz, 1H), 7.94-7.83 (m, 3H), 7.73 (d, J=8.4 Hz, 1H), 7.65 (d, J=8.2 Hz, 2H), 7.61-7.31 (m, 7H), 7.30-7.22 (m, 4H), 7.19-7.12 (m, 1H), 4.87 (d, J=5.0 Hz, 1H), 4.36 (d, J=5.8 Hz, 2H), 4.16-3.82 (m, 6H), 3.66 (br t, J=6.4 Hz, 3H), 3.58-3.48 (m, 6H), 3.45-3.38 (m, 2H), 3.31-3.11 (m, 2H), 2.94-2.82 (m, 1H), 2.63-2.55 (m, 2H), 2.41 (br t, J=6.4 Hz, 3H), 2.21 (s, 3H), 1.54-1.48 (m, 2H), 1.41-1.28 (m, 2H), 1.24-1.14 (m, 6H) SFC: SFC: Retention time=0.418 min; OJ-3-MeOH+ACN (DEA)-40-3ML-35T

### Example 244. Synthesis of (R)-3-(6-(1-(2,2-difluorobenzo[d][1,3]dioxol-5-yl)cyclopropane-1-carboxamido)-3-methylpyridin-2-yl)-N-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)benzamide (Compound 165)

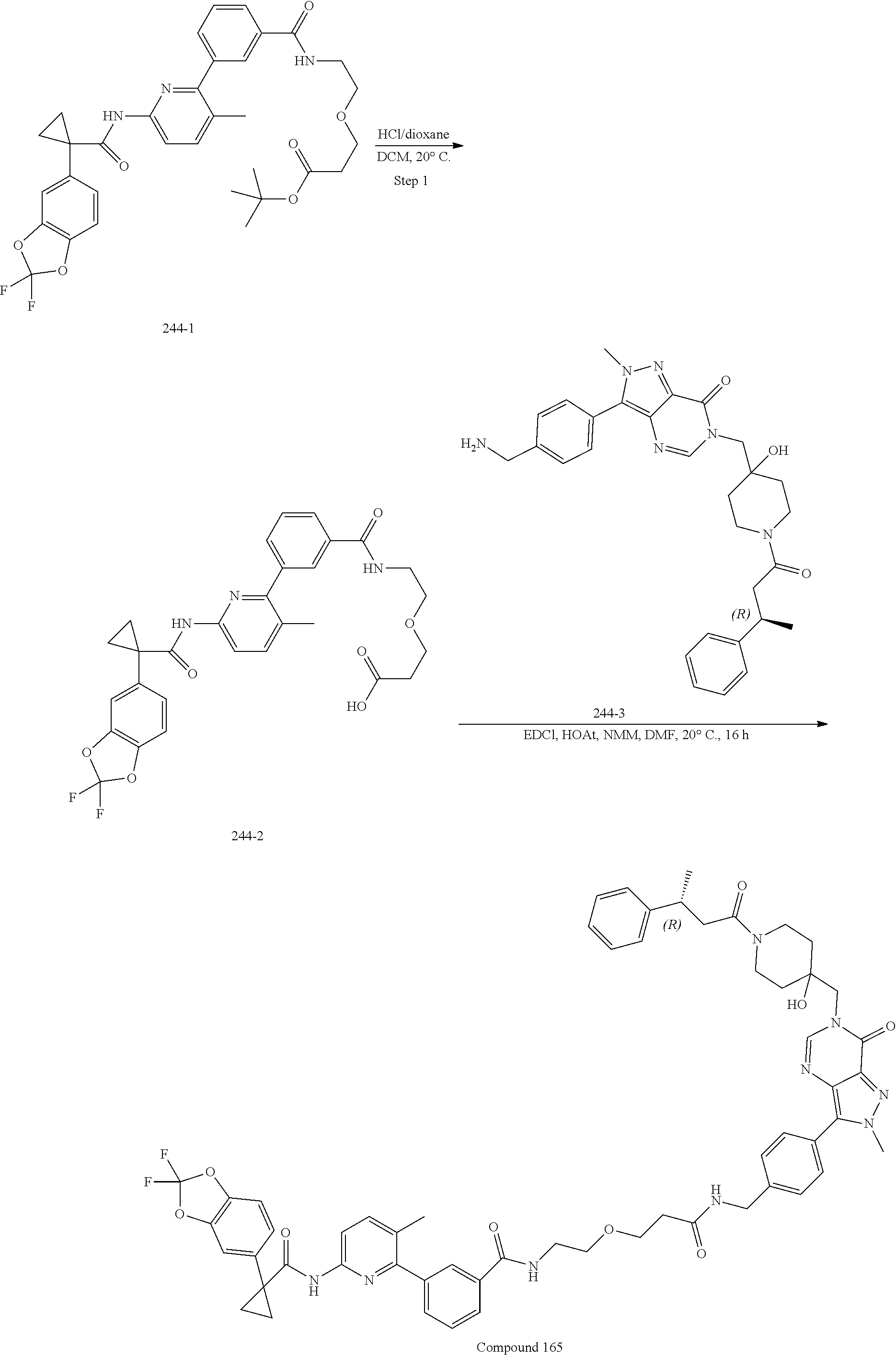

### Step 1: Synthesis of Intermediate 244-2

Detailed Synthetic Procedure: To a solution of Intermediate 244-1 (60 mg, 96.21 umol, 1 eq) in DCM (0.3 mL) was added HCl/dioxane (4 M, 962.09 uL, 40 eq). The mixture was stirred at 20° C. for 1 h. LC-MS showed desired compound was detected. The reaction mixture was concentrated under reduced pressure to give a residue. The crude product was used into the next step without further purification. Intermediate 244-2 (55 mg, crude) was obtained as a white solid.

### Mass Found:

Retention time: 1.041 min, (M+H)=582.2

### Step 2: Synthesis of Compound 165

Detailed Synthetic Procedure: To a solution of Intermediate 244-2 (55 mg, 96.91 umol, 1 eq) Intermediate 244-3 (58.75 mg, 106.60 umol, 1.1 eq, HCl) in DMF (1 mL) was added EDCI (37.16 mg, 193.82 umol, 2 eq) NN (49.01 mg, 484.55 umol, 53.27 uL, 5 eq) and HOAt (13.19 mg, 96.91 umol, 13.56 uL, 1 eq). The mixture was stirred at 20° C. for 16 hr. LC-MS (EW33835-57-P1C2) showed desired compound was detected. The reaction mixture was diluted with MeOH and purified by prep-HPLC directly (column: Phenomenex luna C18 150*25 mm*10 um; mobile phase: [water (FA)-ACN]; B %: 53%-83%, 10 min). Compound 165 (23.73 mg, 20.74 umol, 21.40% yield, 93% purity) was obtained as a white solid confirmed by 1HNMR, LCMS, and SFC.

### Mass Found:

Retention time: 0.891 min, (M+H)=1064.4

Retention time: 0.869 min, (M+H)=1064.5

### NMR Data:

1H NMR (400 MHz, DMSO-d₆) δ=8.95 (br s, 1H), 8.65-8.33 (m, 2H), 8.01-7.82 (m, 4H), 7.72 (br d, J=8.2 Hz, 1H), 7.67-7.61 (m, 2H), 7.59-7.46 (m, 3H), 7.45-7.31 (m, 4H), 7.30-7.10 (m, 5H), 4.87 (br s, 1H), 4.43-4.24 (m, 2H), 4.17-3.85 (m, 6H), 3.77-3.38 (m, 8H), 3.24-3.09 (m, 2H), 2.96-2.80 (m, 1H), 2.58 (br d, J=7.6 Hz, 2H), 2.20 (br s, 3H), 1.59-1.03 (m, 12H)

### SFC:

Rt=0.809 min; method details: Column: Chiralcel OD-3 50×4.6 mm I.D., 3 um Mobile phase: Phase A for CO₂, and Phase B for MeOH+CAN (0.05% DEA); Gradient elution: 60% MeOH+ACN (0.05% DEA) in CO₂ Flow rate: 3 mL/min; Detector: PDAColumn Temp: 35C; Back Pressure: 100Bar

### Example 245. Synthesis of [5-(1-((9-ethyl-7-(4-methylthiophen-2-yl)-9H-carbazol-3-yl)methyl)piperidin-4-yl)-N-(6-(thiophene-2-sulfonamido)benzo[d]thiazol-2-yl)-5,6,7,8-tetrahydro-4H-pyrazolo[1,5-a][1,4]diazepine-2-carboxamide] (Compound 166)

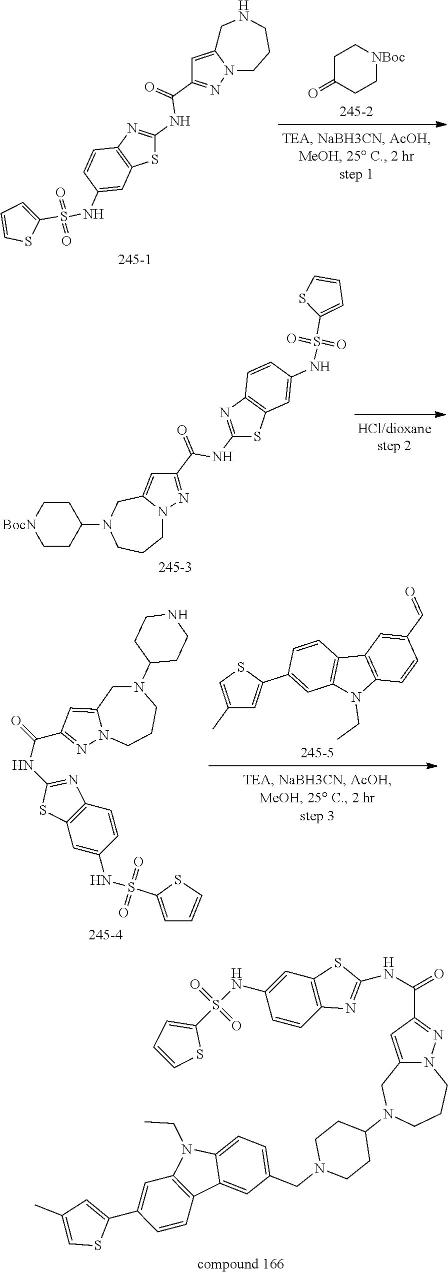 [SMILES: O=C(Nc1nc2ccc(NS(=O)(=O)c3cccs3)cc2s1)c1cc2n(n1)CCCNC2 ; CC(C)(C)OC(=O)N1CCC(=O)CC1 ; CC(C)(C)OC(=O)N1CCC(N2CCCn3nc(C(=O)Nc4nc5ccc(NS(=O)(=O)c6cccs6)cc5s4)cc3C2)CC1 ; O=C(Nc1nc2ccc(NS(=O)(=O)c3cccs3)cc2s1)c1cc2n(n1)CCCN(C1CCNCC1)C2 ; CCn1c2ccc(C=O)cc2c2ccc(-c3cc(C)cs3)cc21 ; CCn1c2ccc(CN3CCC(N4CCCn5nc(C(=O)Nc6nc7ccc(NS(=O)(=O)c8cccs8)cc7s6)cc5C4)CC3)cc2c2ccc(-c3cc(C)cs3)cc21]

### Step 1: Synthesis of Intermediate 245-3

Detailed Synthetic Procedure: To a solution of intermediate 245-1 (100 mg, 195.68 umol, 1 eq) in MeOH (1 mL) was added AcOH (70.50 mg, 1.17 mmol, 67.15 uL, 6 eq), intermediate 245-2 (233.93 mg, 1.17 mmol, 6 eq) and NaBH3CN (73.78 mg, 1.17 mmol, 6 eq). The mixture was stirred at 25° C. for 4 hr. The mixture was washed with H₂O (3 ml), and extracted with DCM 18 ml (3×6 mL). The organic phase was concentrated under reduced pressure to give a white solid which was purified by column chromatography (SiO₂, MeOH/DCM=0%˜20%) to give intermediate 245-3 (200 mg, 191.54 umol, 97.88% yield, 63% purity) as a white solid which was confirmed by LCMS.

### Mass Found:

Retention time: 0.393 min, (M+H)=658.3

Retention time: 0.407 min, (M+H)=658.3

### Step 2: Synthesis of Intermediate 245-4

Detailed Synthetic Procedure: To a solution of intermediate 245-3 (200 mg, 304.03 umol, 1 eq) in HCl/dioxane (2 mL, 4M). The mixture was stirred at 25° C. for 1 hr. The reaction mixture was concentrated under reduced pressure to get intermediate 245-4 (200 mg, crude, HCl) was obtained as a white solid.

### Mass Found:

Retention time: 0.308 min, (M+H)=558.1

### Step 3: Synthesis of Compound 166

Detailed Synthetic Procedure: To a solution of intermediate 245-4 (100 mg, 179.30 umol, 1 eq) in MeOH (1 mL) was added TEA (72.57 mg, 717.22 umol, 99.83 uL, 4 eq), the mixture was stirred at 25° C. for 0.5 hr. Then the mixture was added intermediate 245-5 (91.64 mg, 286.89 umol, 1.6 eq) AcOH (64.61 mg, 1.08 mmol, 61.53 uL, 6 eq) and NaBH3CN (67.61 mg, 1.08 mmol, 6 eq). The mixture was stirred at 25° C. for 11.5 hr. The mixture was washed with H₂O (5 ml), and extracted with DCM 18 ml (3×6 mL). The organic phase was concentrated under reduced pressure to give a crude product. Then the crude product was purified by prep-HPLC (column: Phenomenex luna C18 150*25 mm*10 um; mobile phase: [water (FA)-ACN];B %: 25%-55%, 0 0 min). The eluent was concentrated and lyophilized to give Compound 166 (15 mg, 16.37 umol, 9.13% yield, 99% purity, FA) as a white solid which was confirmed by HNMR and LCMS.

### Mass:

Retention time: 0.491 min, (M+H)=861.4

Retention time: 0.482 min, (M+H)=861.5

### NMR Data:

1H NMR (400 MHz, DMSO-d6) δ=8.21 (s, 1H), 8.13 (d, J=8.0 Hz, 1H), 7.99 (s, 1H), 7.86-7.82 (m, 2H), 7.71 (d, J=1.2 Hz, 1H), 7.63 (d, J=8.8 Hz, 1H), 7.54-7.48 (m, 3H), 7.42-7.35 (m, 2H), 7.18-7.07 (m, 3H), 6.89 (s, 1H), 4.49-4.40 (m, 4H), 3.96-3.92 (m, 2H), 3.57 (s, 2H), 3.15-3.11 (m, 2H), 2.89-2.81 (m, 2H), 2.54-2.52 (m, 3H), 2.28 (s, 3H), 1.96-1.87 (m, 2H), 1.81-1.72 (m, 4H), 1.36-1.31 (m, 3H)

### Example 245. Synthesis of (R)-4-((3-(4-((1-(5-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-5-oxopentyl)piperidin-4-yl)amino)-1-(2,2,2-trifluoroethyl)-1H-indol-2-yl)prop-2-yn-1-yl)amino)-3-methoxy-N-methylbenzamide (Compound 167)

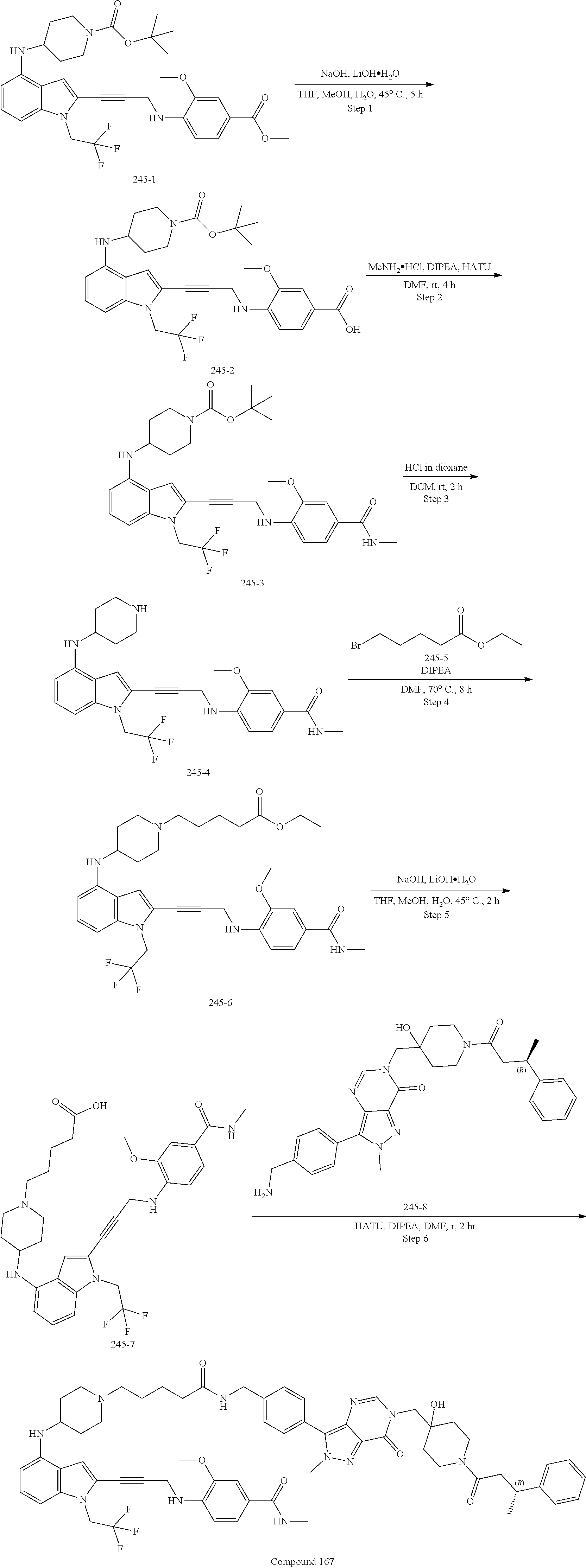

### Step 1: 4-((3-(4-((1-(tert-Butoxycarbonyl)piperidin-4-yl)amino)-1-(2,2,2-trifluoroethyl)-1H-indol-2-yl)prop-2-yn-1-yl)amino)-3-methoxybenzoic acid (245-2)

Into a 50 mL single-necked round-bottomed flask containing a well-stirred solution of tert-butyl 4-((2-(3-((2-methoxy-4-(methoxycarbonyl)phenyl)amino)prop-1-yn-1-yl)-1-(2,2,2-trifluoroethyl)-1H-indol-4-yl)amino)piperidine-1-carboxylate (245-1, 2 g, 3.25 mmol) in MeOH (20 mL), THE (20 mL) and H₂O (10 mL) were added NaOH (1.30 g, 32.5 mmol) and LiOH·H₂O (1.365 g, 32.5 mmol) at room temperature. The resultant mixture was heated at 45° C. for 5 hours before allowed to attain room temperature and concentrated under reduced pressure. The residue was dissolved in H₂O (30 mL) and neutralized to pH ˜7. The solid thus precipitated out was filtered and dried to afford 4-((3-(4-((1-(tert-butoxycarbonyl)piperidin-4-yl)amino)-1-(2,2,2-trifluoroethyl)-1H-indol-2-yl)prop-2-yn-1-yl)amino)-3-methoxybenzoic acid (245-2, 1.8 g, 98% purity, 90% yield) as a light yellow solid. LCMS: 599.2 (M−H), Rt. 2.45 min, 97.89% (Max).

### Step 2: tert-Butyl 4-((2-(3-((2-methoxy-4-(methylcarbamoyl)phenyl)amino)prop-1-yn-1-yl)-1-(2,2,2-trifluoroethyl)-1H-indol-4-yl)amino)piperidine-1-carboxylate (245-3)

Into a 50 mL single-necked round-bottomed flask containing a well-stirred solution of 4-((3-(4-((1-(tert-butoxycarbonyl)piperidin-4-yl)amino)-1-(2,2,2-trifluoroethyl)-1H-indol-2-yl)prop-2-yn-1-yl)amino)-3-methoxybenzoic acid (245-2, 1.9 g, 3.16 mmol) in DMF (20 mL) were added DIPEA (2.11 mL, 12.0 mmol) and HATU (1.80 g, 4.74 mmol) at 25° C. The resulting mixture was stirred at ambient temperature for 5 min. Subsequently, methanamine hydrochloride (0.84 g, 12.5 mmol) was added and stirring was continued for another 4 hours. Afterwards, the mixture was poured into to ice-cold water (100 mL) and the solid thus precipitated out was filtered and dried to afford tert-butyl 4-((2-(3-((2-methoxy-4-(methylcarbamoyl)phenyl)amino)prop-1-yn-1-yl)-1-(2,2,2-trifluoroethyl)-1H-indol-4-yl)amino)piperidine-1-carboxylate (245-3, 1.92 g, 98% purity, 94% yield) as a brown solid.

LCMS: 614.2 (M+H)⁺, Rt. 2.42 min, 97.89% (Max).

### Step 3: 3-Methoxy-N-methyl-4-((3-(4-(piperidin-4-ylamino)-1-(2,2,2-trifluoroethyl)-1H-indol-2-yl)prop-2-yn-1-yl)amino)benzamide dihydrochloride (245-4)

Into a 25 mL single-necked round-bottomed flask containing a well-stirred solution of tert-butyl 4-((2-(3-((2-methoxy-4-(methylcarbamoyl)phenyl)amino)prop-1-yn-1-yl)-1-(2,2,2-trifluoroethyl)-1H-indol-4-yl)amino)piperidine-1-carboxylate (245-3, 1.7 g, 2.77 mmol) in DCM (20 mL) was added HCl (4 M in 1,4-dioxane, 15 mL, 60.0 mmol) at 25° C. The resulting mixture was stirred at room temperature for 2 hours and concentrated under reduced pressure. The crude material was triturated with MTBE (25 mL) to afford 3-methoxy-N-methyl-4-((3-(4-(piperidin-4-ylamino)-1-(2,2,2-trifluoroethyl)-1H-indol-2-yl)prop-2-yn-1-yl)amino)benzamide dihydrochloride (245-4, 1.4 g, 96% purity, 78% yield) as a light brown solid.

LCMS: 514.0 (M+H)⁺, Rt. 2.62 min, 96.15% (Max).

### Step 4: Ethyl 5-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)pentanoate (245-6)

Into a 25 mL single-necked round-bottomed flask containing a well-stirred solution of 3-methoxy-N-methyl-4-((3-(4-(piperidin-4-ylamino)-1-(2,2,2-trifluoroethyl)-1H-indol-2-yl)prop-2-yn-1-yl)amino)benzamide dihydrochloride (245-4, 230 mg, 0.39 mmol) in anhydrous DMF (5 mL) were added DIPEA (507 mg, 3.92 mmol, 0.68 mL) and ethyl 5-bromopentanoate (245-5, 123 mg, 0.59 mmol) at room temperature. The resultant solution was heated at 70° C. for 8 hours. Afterwards, the mixture was cooled to ambient temperature and diluted with water (10 mL). The aqueous layer was extracted with EtOAc (3×10 mL). The combined organic layers were washed with water (2×10 mL), brine (10 mL), dried (anhydrous Na₂SO₄), filtered and concentrated under reduced pressure to afford the crude material. Purification by a flash silica-gel (230-400 mesh) column chromatography (0-10% MeOH/DCM) afforded ethyl 5-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)pentanoate (245-6, 175 mg, 95% purity, 66% yield) as a light brown solid.

UP-LCMS: 642.1 (M+H)⁺, Rt. 1.70 min, 94.78% (Max).

### Step 5: 5-(4-((2-(3-((2-Methoxy-4-(methylcarbamoyl)phenyl)amino)prop-1-yn-1-yl)-1-methyl-1H-indol-4-yl)amino)piperidin-1-yl)pentanoic acid (245-7)

Into a 25 mL single-necked round-bottomed flask containing a well-stirred solution of ethyl 5-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)pentanoate (245-6, 150 mg, 0.23 mmol) in MeOH (3 mL), THE (3 mL) and H₂O (3 mL) were added NaOH (93 mg, 2.33 mmol) LiOH·H₂O (98 mg, 2.33 mmol) at room temperature. The resultant mixture was heated at 45° C. for 2 hours before allowed to attain room temperature and concentrated under reduced pressure. The residue was dissolved in H₂O (10 mL) and neutralized to pH ˜7. The solid thus precipitated out was filtered and dried to afford 5-(4-((2-(3-((2-methoxy-4-(methylcarbamoyl)phenyl)amino)prop-1-yn-1-yl)-1-methyl-1H-indol-4-yl)amino)piperidin-1-yl)pentanoic acid (245-7, 125 mg, 97% purity, 84% yield) as yellow solid. LCMS: 614.3 (M+H)⁺, Rt. 1.56 min, 97.54% (Max).

### Step 6: (R)-4-((3-(4-((1-(5-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-5-oxopentyl)piperidin-4-yl)amino)-1-(2,2,2-trifluoroethyl)-1H-indol-2-yl)prop-2-yn-1-yl)amino)-3-methoxy-N-methylbenzamide (Compound 167)

Into a 25 mL single-necked round-bottomed flask containing a well-stirred solution of 5-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)pentanoic acid (7, 125 mg, 0.20 mmol) in anhydrous DMF (3 mL) were added DIPEA (0.35 mL, 2.03 mmol) and HATU (116 mg, 0.30 mmol) at room temperature. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (245-8, 115 mg, 0.22 mmol) was added and stirring was continued for another 2 hours. Afterwards, the mixture was concentrated under reduced pressure to afford the crude material that was purified by a reversed-phase preparatory HPLC [Column: X-Bridge C8 (19×150) mm, 5 μm; Mobile phase A: 10 mM Ammonium bicarbonate in water and Mobile phase B: Acetonitrile; Flow rate: 12 mL/min] to afford (R)-4-((3-(4-((1-(5-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-5-oxopentyl)piperidin-4-yl)amino)-1-(2,2,2-trifluoroethyl)-1H-indol-2-yl)prop-2-yn-1-yl)amino)-3-methoxy-N-methylbenzamide (Compound 167, 65 mg, 99.9% purity, 29% yield) as a light yellow solid.

¹H NMR (300 MHz, DMSO-d₆) δ=8.41 (t, J=5.7 Hz, 1H), 8.14-8.06 (m, 1H), 7.97 (d, J=7.5 Hz, 1H), 7.67 (d, J=8.3 Hz, 2H), 7.48-7.40 (m, 3H), 7.37-7.33 (m, 1H), 7.30-7.22 (m, 4H), 7.20-7.12 (m, 1H), 7.06 (s, 1H), 6.98 (t, J=7.9 Hz, 1H), 6.76 (d, J=8.3 Hz, 1H), 6.67 (d, J=8.3 Hz, 1H), 6.14 (d, J=8.0 Hz, 1H), 5.98 (t, J=6.3 Hz, 1H), 5.46 (d, J=7.8 Hz, 1H), 4.98-4.83 (m, 3H), 4.34 (dd, J=6.0, 13.3 Hz, 4H), 4.10 (s, 3H), 4.05-3.94 (m, 2H), 3.90 (br s, 1H), 3.84 (s, 3H), 3.73-3.57 (m, 1H), 3.24-3.13 (m, 2H), 2.95-2.82 (m, 3H), 2.76 (d, J=4.4 Hz, 3H), 2.67-2.55 (m, 3H), 2.35-2.25 (m, 3H), 2.20 (t, J=7.0 Hz, 2H), 2.07-1.87 (m, 4H), 1.63-1.26 (m, 11H), 1.21 (d, J=6.9 Hz, 3H). LCMS: (Method A) 1110.3 (M+H)⁺, Rt. 1.78 min, 99.95% (Max); HPLC: (Method A) Rt. 4.10 min, 99.84% (Max).

### Example 246. Synthesis of (R)-4-((3-(4-((1-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropyl)piperidin-4-yl)amino)-1-(2,2,2-trifluoroethyl)-1H-indol-2-yl)prop-2-yn-1-yl)amino)-3-methoxy-N-methylbenzamide (Compound 168)

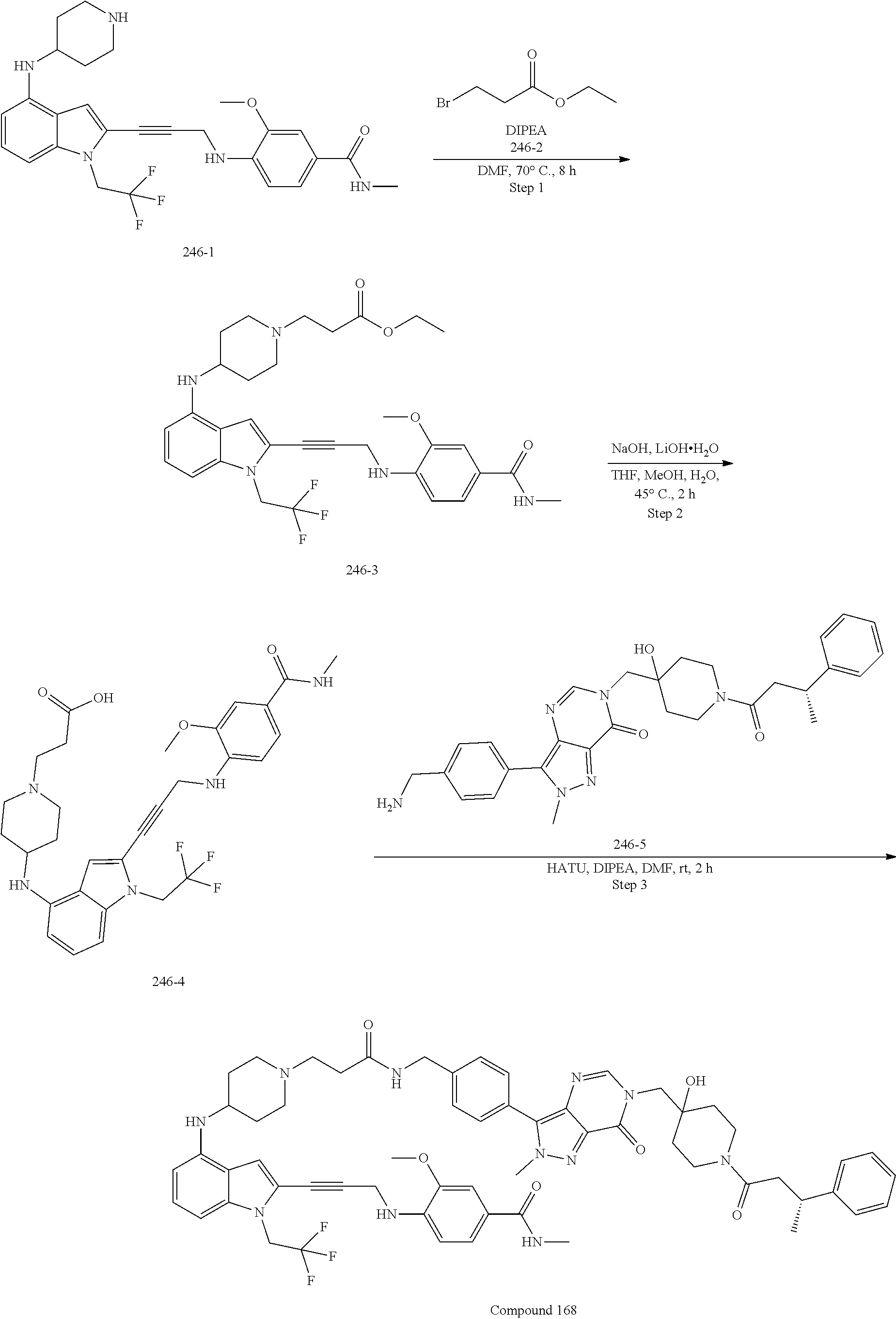

### Step 1: Ethyl 3-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)propanoate (246-3)

Into a single-necked round-bottomed flask containing a well-stirred solution of 3-methoxy-N-methyl-4-((3-(4-(piperidin-4-ylamino)-1-(2,2,2-trifluoroethyl)-1H-indol-2-yl)prop-2-yn-1-yl)amino)benzamide hydrochloride (246-1, 0.25 g, 0.45 mmol) in anhydrous DMF (3 mL) were added DIPEA (1.70 mL, 9.73 mmol) and ethyl 3-bromopropanoate (246-2, 0.22 g, 1.21 mmol) at room temperature. The resultant solution was heated at 70° C. for 8 hours. Afterwards, the mixture was cooled to ambient temperature and diluted with water (15 mL). The aqueous layer was extracted with EtOAc (3×15 mL). The combined organic layers were washed with water (2×15 mL), brine (15 mL), dried (anhydrous Na₂SO₄), filtered and concentrated under reduced pressure to afford the crude material. Purification by a flash silica-gel (230-400 mesh) column chromatography (5-10% MeOH/DCM) afforded ethyl 3-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)propanoate (246-3, 0.13 g, 87% purity, 40% yield) as a brown solid.

LCMS: 614.1 (M+H)⁺, Rt. 2.77 min, 86.49% (Max).

### Step 2: 3-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)propanoic acid (246-4)

Into a 25 mL single-necked round-bottomed flask containing a well-stirred solution of ethyl 3-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)propanoate (246-3, 0.13 g, 0.21 mmol) in MeOH (3 mL), THE (3 mL) and H₂O (3 mL) were added NaOH (0.085 g, 2.12 mmol) and LiOH·H₂O (0.05 g, 2.12 mmol)) at room temperature. The resultant mixture was heated at 45° C. for 2 hours before allowed to attain room temperature and concentrated under reduced pressure. The residue was dissolved in H₂O (10 mL) and neutralized to pH ˜6. The solid thus precipitated out was filtered and dried to afford 3-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)propanoic acid (246-4, 0.1 g, 98% purity, 79% yield).

LCMS: 586.3 (M+H)⁺, Rt. 1.49 min, 97.63% (Max).

### Step 3: (R)-4-((3-(4-((1-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropyl)piperidin-4-yl)amino)-1-(2,2,2-trifluoroethyl)-1H-indol-2-yl)prop-2-yn-1-yl)amino)-3-methoxy-N-methylbenzamide (246-6)

Into a 25 mL single-necked round-bottomed flask containing a well-stirred 3-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)propanoic acid (246-4, 0.1 g, 0.17 mmol) in anhydrous DMF (2 mL) were added DIPEA (0.3 mL, 1.71 mmol) and HATU (0.097 g, 0.26 mmol) at room temperature. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 0.105 g, 0.20 mmol) was added and stirring was continued for another 2 hours. Afterwards, the mixture was concentrated under reduced pressure to afford the crude material that was purified by a reversed-phase preparatory HPLC [Column: X-Select C18 (250×21.2 mm) 5 μm; Mobile phase A: 10 mM Ammonium bicarbonate in water and Mobile phase B: Acetonitrile; Flow rate-12 mL/min] to afford (R)-4-((3-(4-((1-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropyl)piperidin-4-yl)amino)-1-(2,2,2-trifluoroethyl)-1H-indol-2-yl)prop-2-yn-1-yl)amino)-3-methoxy-N-methylbenzamide (Compound 168, 22.51 mg, 98.7% purity, 12% yield) as light yellow solid.

¹H NMR (400 MHz, DMSO-d₆) δ=8.55 (t, J=5.4 Hz, 1H), 8.13-8.07 (m, 1H), 7.97 (d, J=10.1 Hz, 1H), 7.68 (d, J=8.3 Hz, 2H), 7.48 (d, J=8.3 Hz, 2H), 7.44-7.40 (m, 1H), 7.36-7.34 (m, 1H), 7.29-7.24 (m, 4H), 7.19-7.14 (m, 1H), 7.05 (s, 1H), 6.99 (t, J=8.1 Hz, 1H), 6.76 (d, J=8.4 Hz, 1H), 6.68 (d, J=8.5 Hz, 1H), 6.16 (d, J=8.0 Hz, 1H), 5.99 (t, J=6.3 Hz, 1H), 5.48 (d, J=8.4 Hz, 1H), 4.95-4.85 (m, 3H), 4.38 (d, J=5.5 Hz, 2H), 4.32 (d, J=6.3 Hz, 2H), 4.08 (s, 3H), 4.06-3.96 (m, 2H), 3.94-3.88 (m, 1H), 3.84 (s, 3H), 3.72-3.59 (m, 1H), 3.24-3.10 (m, 2H), 2.96-2.81 (m, 2H), 2.75 (d, J=4.4 Hz, 3H), 2.66-2.56 (m, 2H), 2.42-2.35 (m, 4H), 2.18-2.05 (m, 3H), 2.00-1.91 (m, 2H), 1.55-1.42 (m, 3H), 1.42-1.28 (m, 3H), 1.21 (d, J=6.9 Hz, 3H).

LCMS: (Method D) 1082.6 (M+H)⁺, Rt. 2.36 min, 98.71% (Max). HPLC: Rt. 6.20 min, 98.68% (Max).

### Example 246. Synthesis of (R)-4-((3-(4-((1-(2-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)piperidin-4-yl)amino)-1-(2,2,2-trifluoroethyl)-1H-indol-2-yl)prop-2-yn-1-yl)amino)-3-methoxy-N-methylbenzamide (Compound 169)

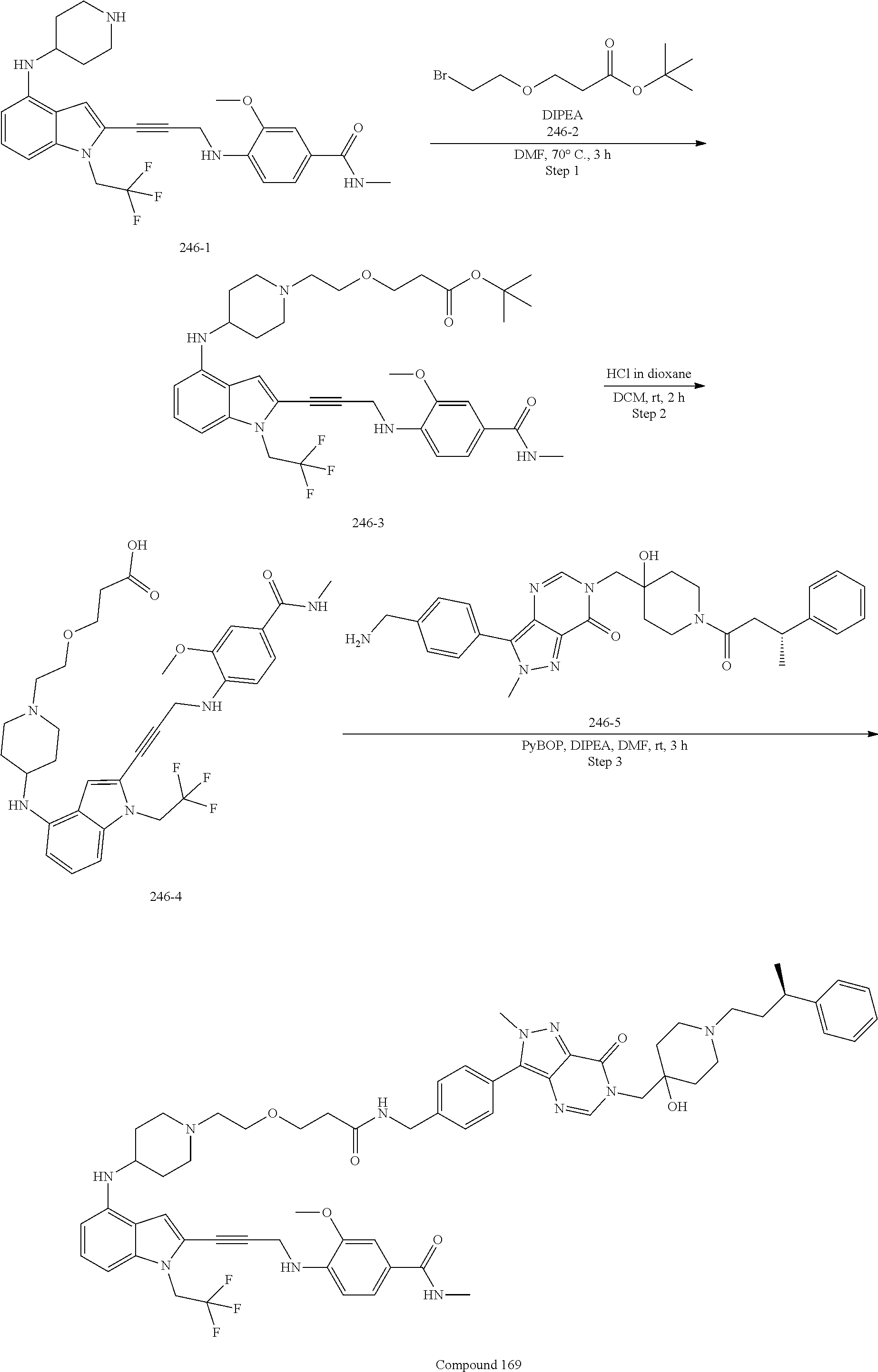

### Step 1: tert-Butyl 3-(2-(4-((2-(3-((2-methoxy-4-(methylcarbamoyl)phenyl)amino)prop-1-yn-PP-1 34c 3 1-yl)-1-(2,2,2-trifluoroethyl)-1H-indol-4-yl)amino)piperidin-1-yl)ethoxy)propanoate (246-3)

Into a 10 mL single-necked round-bottomed flask containing a well-stirred solution of 3-methoxy-N-methyl-4-((3-(4-(piperidin-4-ylamino)-1-(2,2,2-trifluoroethyl)-1H-indol-2-yl)prop-2-yn-1-yl)amino)benzamide hydrochloride (246-1, 0.25 g, 0.45 mmol) in anhydrous DMF (3.0 mL) were added DIPEA (1.70 mL, 9.73 mmol) and tert-butyl 3-(2-bromoethoxy)propanoate (246-2, 0.37 g, 1.45 mmol) at room temperature. The resultant solution was heated at 70° C. for 3 hours. Afterwards, the mixture was cooled to ambient temperature and diluted with water (15 mL). The aqueous layer was extracted with EtOAc (3×15 mL). The combined organic layers were washed with water (2×15 mL), brine (15 mL), dried (anhydrous Na₂SO₄), filtered and concentrated under reduced pressure to afford the crude material. Purification by a flash silica-gel (230-400 mesh) column chromatography (5-10% MeOH/DCM) afforded tert-butyl 3-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)propanoate (246-3, 0.14 g, 95% purity, 43% yield) as a brown solid. LCMS: 686.1 (M+H)⁺, Rt. 2.98 min, 95.32% (Max).

### Step 2: 3-(2-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)ethoxy)propanoic acid hydrochloride (246-4)

Into a 25 mL single-necked round-bottomed flask containing a well-stirred solution of tert-butyl 3-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)propanoate (246-3, 0.14 g, 0.20 mmol) in DCM (3 mL) was added HCl (4M in dioxane, 1.3 mL, 5.10 mmol)) at 25° C. The resulting mixture was stirred at room temperature for 2 hours and concentrated under reduced pressure. The crude material was triturated with MTBE (5 mL) to afford 3-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)propanoic acid hydrochloride (246-4, 0.135 g, 89% purity, 89% yield) as a brown solid. LCMS: 630.0 (M+H)⁺, Rt. 2.13 min, 89.51 (Max).

### Step 3: (R)-4-((3-(4-((1-(2-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)piperidin-4-yl)amino)-1-(2,2,2-trifluoroethyl)-1H-indol-2-yl)prop-2-yn-1-yl)amino)-3-methoxy-N-methylbenzamide (Compound 169)

Into a 25 mL single-necked round-bottomed flask containing a well-stirred solution 3-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)propanoic acid hydrochloride (246-4, 0.1 g, 0.15 mmol) in anhydrous DMF (3 mL) were added DIPEA (0.26 mL, 1.50 mmol) and PyBOP (0.117 g, 0.225 mmol) at room temperature. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 0.093 g, 0.18 mmol) was added and stirring was continued for another 3 hours. Afterwards, the mixture was concentrated under reduced pressure to afford the crude material that was purified by a reversed-phase preparatory HPLC [Column: KROMOSIL-C18 (250×21.2 mm) 5 μm; Mobile phase A: 10 mM Ammonium bicarbonate in H₂O and Mobile phase B: Acetonitrile; Flow Rate: 15 mL/min] to afford (R)-4-((3-(4-((1-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)piperidin-4-yl)amino)-1-(2,2,2-trifluoroethyl)-1H-indol-2-yl)prop-2-yn-1-yl)amino)-3-methoxy-N-methylbenzamide (Compound 169, 24.8 mg, 98.5% purity, 14% yield) as a pale yellow solid.

¹H NMR (400 MHz, DMSO-d₆) δ=8.48 (t, J=6.0 Hz, 1H), 8.11 (q, J=4.3 Hz, 1H), 7.97 (d, J=10.0 Hz, 1H), 7.66 (d, J=8.0 Hz, 2H), 7.48-7.39 (m, 3H), 7.35 (d, J=2.0 Hz, 1H), 7.30-7.22 (m, 5H), 7.20-7.12 (m, 1H), 7.05 (s, 1H), 6.97 (t, J=8.0 Hz, 1H), 6.75 (d, J=8.5 Hz, 1H), 6.67 (d, J=8.0 Hz, 1H), 6.12 (d, J=8.0 Hz, 1H), 5.99 (t, J=6.5 Hz, 1H), 5.45 (d, J=8.0 Hz, 1H), 4.95-4.84 (m, 3H), 4.38 (d, J=5.5 Hz, 2H), 4.31 (d, J=6.0 Hz, 2H), 4.09 (s, 3H), 4.07-3.94 (m, 3H), 3.84 (s, 3H), 3.66 (t, J=6.3 Hz, 2H), 3.51 (t, J=6.0 Hz, 2H), 3.24-3.10 (m, 2H), 3.05-2.99 (m, 1H), 2.92-2.83 (m, 3H), 2.75 (d, J=4.5 Hz, 3H), 2.62-2.56 (m, 2H), 2.46-2.40 (m, 3H), 2.16-2.05 (m, 2H), 1.94-1.86 (m, 2H), 1.56-1.26 (m, 6H), 1.21 (d, J=7.0 Hz, 3H). LCMS: 1127.4 (M+H)⁺, Rt. 1.73 min, 98.49% (Max). HPLC: Rt. 4.03 min, 95.62% (Max).

### Example 247. Synthesis of (R)-4-((3-(4-((1-(2-(2-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethoxy)ethyl)piperidin-4-yl)amino)-1-(2,2,2-trifluoroethyl)-1H-indol-2-yl)prop-2-yn-1-yl)amino)-3-methoxy-N-methylbenzamide (Compound 170)

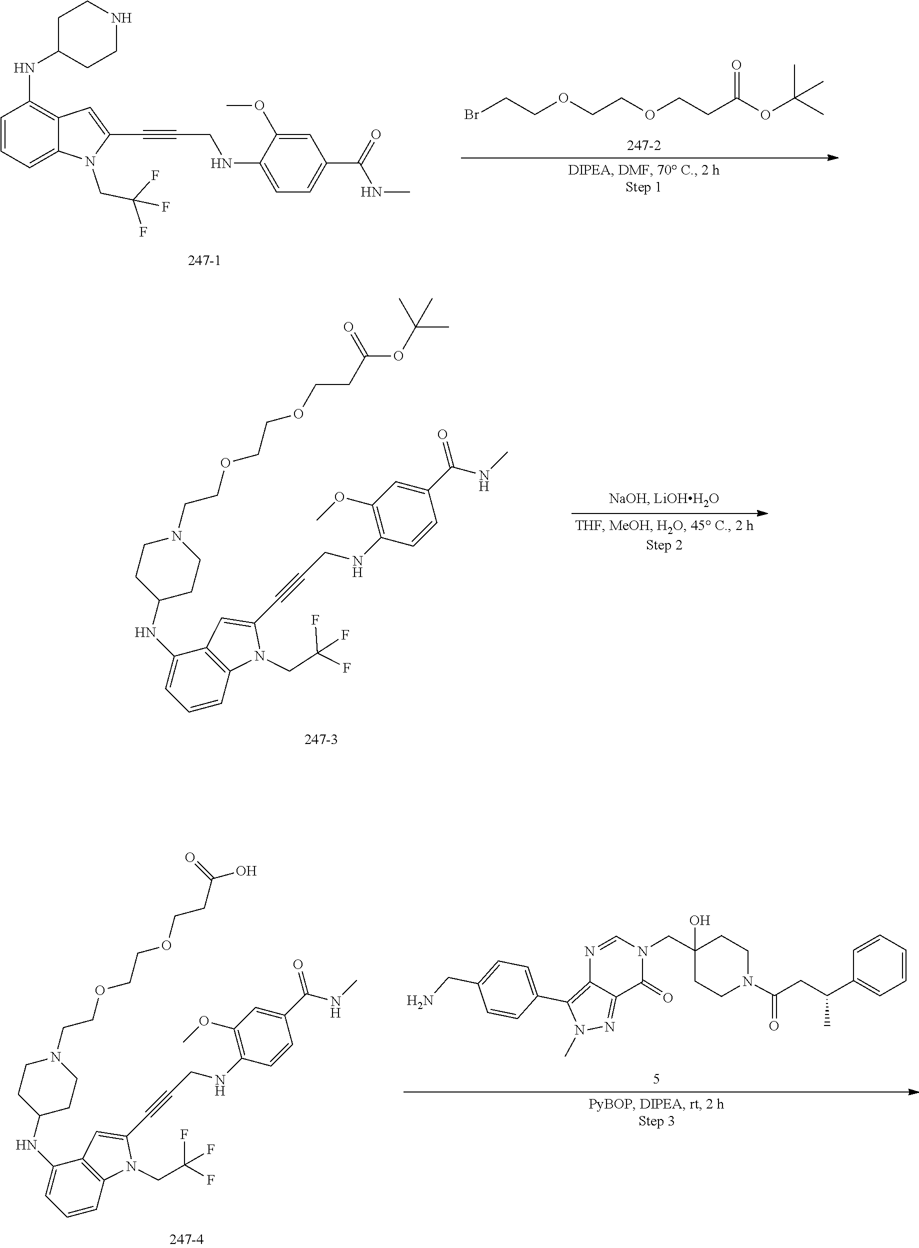 [SMILES: CNC(=O)c1ccc(NCC#Cc2cc3c(NC4CCNCC4)cccc3n2CC(F)(F)F)c(OC)c1 ; CC(C)(C)OC(=O)CCOCCOCCBr ; CNC(=O)c1ccc(NCC#Cc2cc3c(NC4CCN(CCOCCOCCC(=O)OC(C)(C)C)CC4)cccc3n2CC(F)(F)F)c(OC)c1 ; CNC(=O)c1ccc(NCC#Cc2cc3c(NC4CCN(CCOCCOCCC(=O)O)CC4)cccc3n2CC(F)(F)F)c(OC)c1 ; C[C@H](CC(=O)N1CCC(O)(Cn2cnc3c(-c4ccc(CN)cc4)n(C)nc3c2=O)CC1)c1ccccc1]

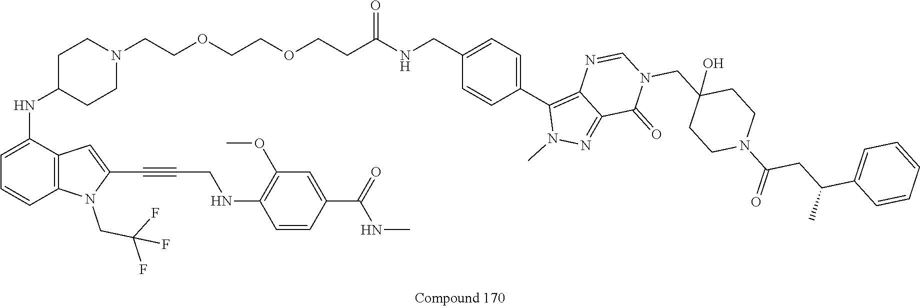 [SMILES: CNC(=O)c1ccc(NCC#Cc2cc3c(NC4CCN(CCOCCOCCC(=O)NCc5ccc(-c6c7ncn(CC8(O)CCN(C(=O)C[C@@H](C)c9ccccc9)CC8)c(=O)c7nn6C)cc5)CC4)cccc3n2CC(F)(F)F)c(OC)c1]

### Step 1. tert-Butyl 3-(2-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)ethoxy)propanoate (247-3)

In a 30 mL pressure-relief vial containing a well-stirred solution of 3-methoxy-N-methyl-4-((3-(4-(piperidin-4-ylamino)-1-(2,2,2-trifluoroethyl)-1H-indol-2-yl)prop-2-yn-1-yl)amino)benzamide hydrochloride (247-1, 300 mg, 0.54 mmol) in DMF (3 mL) were added DIPEA (2.0 mL, 11.7 mmol) and tert-butyl 3-(2-(2-bromoethoxy)ethoxy)propanoate (247-2, 405 mg, 1.36 mmol). The resultant mixture was heated at 70° C. for 2 hours and then allowed to attain room temperature. Afterwards, the mixture was diluted with water (15 mL) and extracted with EtOAc (2×10 mL). The combined organic layers were washed with brine (30 mL), dried over anhydrous sodium sulphate, filtered and concentrated under reduced pressure to get the crude material that was purified by a flash silica-gel (230-400 mesh) column chromatography (10% DCM in methanol) to afford tert-butyl 3-(2-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)ethoxy)propanoate (247-3, 200 mg, 98.6% purity, 49% yield) as a brown solid. LCMS: 730.4 (M+H)⁺, Rt. 1.88 min, 98.66% (Max).

### Step 2: 3-(2-(2-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)ethoxy)ethoxy)propanoic acid (247-4)

In a 30 mL pressure-relief vial containing a well-stirred solution of tert-butyl 3-(2-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)ethoxy)propanoate (247-3, 200 mg, 0.27 mmol) in THE (2 mL), methanol (2.0 mL) and water (2.0 mL) were added NaOH (110 mg, 2.74 mmol) and LiOH·H₂O (65.6 mg, 2.74 mmol) at room temperature The resultant mixture was heated at 45° C. for 5 hours before allowed to attain room temperature, diluted with H₂O (30 mL) and neutralized to pH ˜7. The mixture was extracted with 5% MeOH in DCM (2×10 mL). The combined organic layers were washed with brine (30 mL), dried over anhydrous sodium sulphate, filtered and concentrated under reduced pressure to get the crude 3-(2-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)ethoxy)propanoic acid (247-4, 100 mg, 97% purity, 52.5% yield) as a brown solid. LCMS: 674.3 (M+H)⁺, Rt. 1.56 min, 96.93% (Max).

### Step 3. (R)-4-((3-(4-((1-(2-(2-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethoxy)ethyl)piperidin-4-yl)amino)-1-(2,2,2-trifluoroethyl)-1H-indol-2-yl)prop-2-yn-1-yl)amino)-3-methoxy-N-methylbenzamide (Compound 170)

In a 10 mL pressure-relief vial containing a well-stirred solution of, 3-(2-(2-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)ethoxy)ethoxy)propanoic acid (247-4, 60 mg, 0.09 mmol) in DMF (1.0 mL) was added DIPEA (115 mg, 0.89 mmol) and PyBOP (69.5 mg, 0.13 mmol) at room temperature. After 15 mins, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (247-5, 50 mg, 0.10 mmol) was added and stirring was continued for another 2 hours. Afterwards, solvent was evaporated under reduced pressure and the residue was purified by a reversed-phase preparatory HPLC [Column: EVO-C18 (250×21.2 mm) 5 μm; Mobile Phase A: 10 mM Ammonium bicarbonate in water and Mobile Phase B: acetonitrile] to afford (R)-4-((3-(4-((1-(2-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethoxy)ethyl)piperidin-4-yl)amino)-1-(2,2,2-trifluoroethyl)-1H-indol-2-yl)prop-2-yn-1-yl)amino)-3-methoxy-N-methylbenzamide (Compound 170, 22.64 mg, 21% yield, 96.6% purity) as a light yellow solid.

1H NMR (400 MHz, DMSO-d₆) δ=8.47 (t, J=5.8 Hz, 1H), 8.15-8.07 (m, 1H), 7.97 (d, J=10.0 Hz, 1H), 7.66 (d, J=8.0 Hz, 2H), 7.49-7.39 (m, 3H), 7.35 (s, 1H), 7.30-7.21 (m, 5H), 7.19-7.12 (m, 1H), 7.06 (s, 1H), 6.98 (t, J=8.0 Hz, 1H), 6.75 (d, J=8.0 Hz, 1H), 6.67 (d, J=8.5 Hz, 1H), 6.14 (d, J=7.5 Hz, 1H), 6.00 (t, J=6.5 Hz, 1H), 5.46 (d, J=8.0 Hz, 1H), 4.97-4.83 (m, 3H), 4.37 (d, J=5.5 Hz, 2H), 4.31 (d, J=6.5 Hz, 2H), 4.10 (s, 3H), 4.04-3.87 (m, 3H), 3.84 (s, 3H), 3.67 (t, J=6.0 Hz, 3H), 3.54-3.47 (m, 6H), 3.23-3.11 (m, 3H), 2.92-2.82 (m, 3H), 2.75 (d, J=4.5 Hz, 3H), 2.14-2.00 (m, 3H), 1.96-1.83 (m, 3H), 1.54-1.24 (m, 7H), 1.20 (d, J=7.0 Hz, 3H). LCMS: (Method A) 1170.4 (M+H)⁺, Rt. 1.74 min, 96.68% (Max); HPLC: Rt. 3.52 min, 96.48% (Max).

### Example 248. Synthesis of (R)-4-((3-(4-((1-(7-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-7-oxoheptyl)piperidin-4-yl)amino)-1-(2,2,2-trifluoroethyl)-1H-indol-2-yl)prop-2-yn-1-yl)amino)-3-methoxy-N-methylbenzamide (Compound 171)

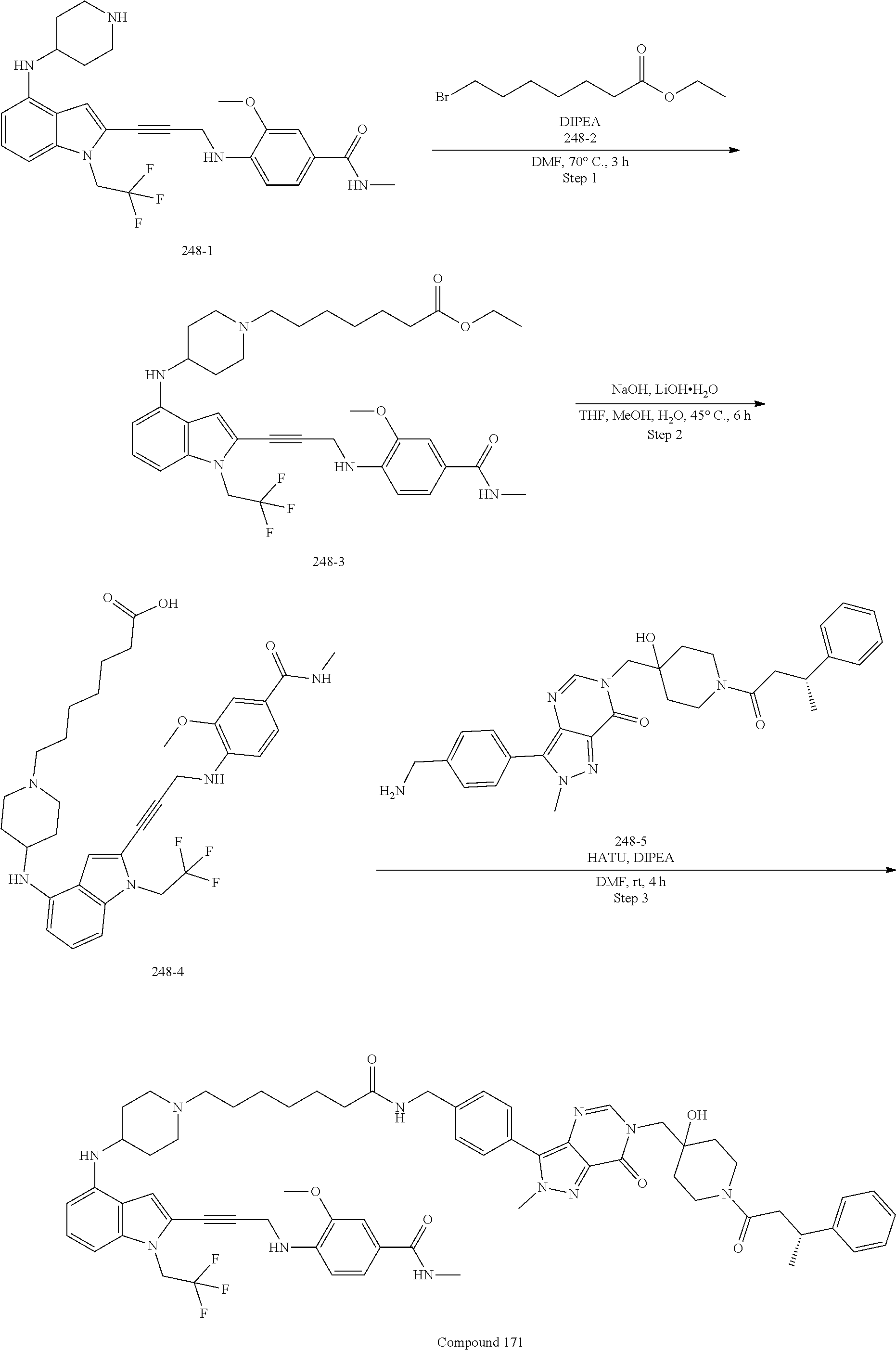 [SMILES: CNC(=O)c1ccc(NCC#Cc2cc3c(NC4CCNCC4)cccc3n2CC(F)(F)F)c(OC)c1 ; CCOC(=O)CCCCCCBr ; CCOC(=O)CCCCCCN1CCC(Nc2cccc3c2cc(C#CCNc2ccc(C(=O)NC)cc2OC)n3CC(F)(F)F)CC1 ; CNC(=O)c1ccc(NCC#Cc2cc3c(NC4CCN(CCCCCCC(=O)O)CC4)cccc3n2CC(F)(F)F)c(OC)c1 ; C[C@H](CC(=O)N1CCC(O)(Cn2cnc3c(-c4ccc(CN)cc4)n(C)nc3c2=O)CC1)c1ccccc1 ; CNC(=O)c1ccc(NCC#Cc2cc3c(NC4CCN(CCCCCCC(=O)NCc5ccc(-c6c7ncn(CC8(O)CCN(C(=O)C[C@@H](C)c9ccccc9)CC8)c(=O)c7nn6C)cc5)CC4)cccc3n2CC(F)(F)F)c(OC)c1]

### Step 1. Ethyl 7-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)heptanoate (248-3)

Into a 25 mL single-necked round-bottomed flask containing a well-stirred solution of 4-((3-(4-((1-imino-1l5-chlorinan-4-yl)amino)-1-(2,2,2-trifluoroethyl)-1H-indol-2-yl)prop-2-yn-1-yl)amino)-3-methoxy-N-methylbenzamide hydrochloride (248-1, 250 mg, 0.42 mmol) in DMF (4 mL), was added DIPEA (1.48 mL, 8.53 mmol). The reaction mixture was stirred at room temperature for 1 h. Subsequently, ethyl 7-bromoheptanoate (248-2, 303 mg, 1.27 mmol) was added and the mixture was heated at 70° C. for another 3 h. After completion, the mixture was cooled to ambient temperature, diluted with water (10 mL) and extracted with EtOAc (3×10 mL). The combined organic layers were washed with water (2×10 mL), brine (10 mL), dried (anhydrous Na₂SO₄), filtered and concentrated under reduced pressure to afford the crude material that was purified by a flash silica-gel (230-400 mesh) column chromatography (10% MeOH/DCM) to afford ethyl 7-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)heptanoate (3, 120 mg, 98% purity, 41% yield) as a brown solid. LCMS: (Method B) 670.3 (M+H)⁺, Rt. 1.82 min, 98.64% (Max);

### Step 2. 7-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)heptanoic acid (248-4)

Into a 25 mL single-necked round-bottomed flask containing a well-stirred solution of ethyl 7-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)heptanoate (248-3, 120 mg, 0.18 mmol) in MeOH (2 mL), THE (2 mL) and H₂O (2 mL), were added NaOH (71.7 mg, 1.80 mmol) and LiOH (42.9 mg, 1.80 mmol) at room temperature. The resultant mixture was heated at 45° C. for 6 hours before allowed to attain room temperature and concentrated under reduced pressure. The residue was diluted with water (10 mL), neutralized to pH ˜7 and then extracted with DCM (2×10 mL). The combined organic layers were washed with brine solution, dried over anhydrous sodium sulphate and concentrated under reduced pressure to the afford crude material that was washed with MTBE to obtain 7-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)heptanoic acid (248-4, 75 mg, 72% purity, 47% yield) as a brown solid. LCMS: (Method C) 642.2 (M+H)⁺, Rt. 1.87 min, 72.46% (Max);

### Step 3. (R)-4-((3-(4-((1-(7-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-7-oxoheptyl)piperidin-4-yl)amino)-1-(2,2,2-trifluoroethyl)-1H-indol-2-yl)prop-2-yn-1-yl)amino)-3-methoxy-N-methylbenzamide (Compound 171)

Into a 25 mL single-necked round-bottomed flask containing a well-stirred solution of 7-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)heptanoic acid (248-4, 75 mg, 0.12 mmol) in DMF (3 mL) were added HATU (66.7 mg, 0.17 mmol) and DIPEA (0.204 mL, 1.17 mmol). The reaction mixture was stirred at room temperature for 1 h. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (248-5, 72.2 mg, 0.14 mmol) was added and the reaction was allowed to stir for another 3 h. After completion, the mixture was concentrated under reduced pressure to afford the crude material that was purified by a reversed-phase preparatory HPLC [Column: EVO-C18 (250×21.2 mm) 5 μm; Mobile phase A: 10 mM Ammonium bicarbonate in water and Mobile phase B: Acetonitrile in THF; Flow rate: 15 mL/min] to afford (R)-4-((3-(4-((1-(7-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-7-oxoheptyl)piperidin-4-yl)amino)-1-(2,2,2-trifluoroethyl)-1H-indol-2-yl)prop-2-yn-1-yl)amino)-3-methoxy-N-methylbenzamide (Compound 171, 6 mg, 98% purity, 4% yield) as an off-white solid.

¹H NMR (400 MHz, DMSO-d₆) δ=8.41 (t, J=5.9 Hz, 1H), 8.11 (q, J=4.5 Hz, 1H), 7.98 (d, J=10.1 Hz, 1H), 7.67 (d, J=8.3 Hz, 2H), 7.47-7.39 (m, 3H), 7.35 (d, J=1.8 Hz, 1H), 7.30-7.22 (m, 4H), 7.20-7.13 (m, 1H), 7.06 (s, 1H), 6.99 (t, J=8.0 Hz, 1H), 6.76 (d, J=8.3 Hz, 1H), 6.68 (d, J=8.1 Hz, 1H), 6.14 (d, J=7.9 Hz, 1H), 5.99 (t, J=6.3 Hz, 1H), 5.47 (d, J=7.9 Hz, 1H), 4.95-4.85 (m, 3H), 4.36 (d, J=6.0 Hz, 2H), 4.31 (d, J=6.5 Hz, 2H), 4.10 (s, 3H), 4.07-3.95 (m, 2H), 3.84 (s, 3H), 3.71-3.59 (m, 1H), 3.22-3.12 (m, 2H), 2.93-2.82 (m, 3H), 2.75 (d, J=4.5 Hz, 3H), 2.65-2.56 (m, 2H), 2.31-2.25 (m, 2H), 2.17 (t, J=7.4 Hz, 2H), 2.06-1.97 (m, 2H), 1.96-1.88 (m, 2H), 1.60-1.33 (m, 9H), 1.33-1.23 (m, 7H), 1.21 (d, J=6.9 Hz, 3H). LCMS: (Method A) 1139.4 (M+H)⁺, Rt. 1.67 min, 99.61% (Max); HPLC: Rt. 4.23 min, 98.86% (Max);

### Example 249. Synthesis of (R)-4-((3-(4-((1-(9-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-9-oxononyl)piperidin-4-yl)amino)-1-(2,2,2-trifluoroethyl)-1H-indol-2-yl)prop-2-yn-1-yl)amino)-3-methoxy-N-methylbenzamide (Compound 172)

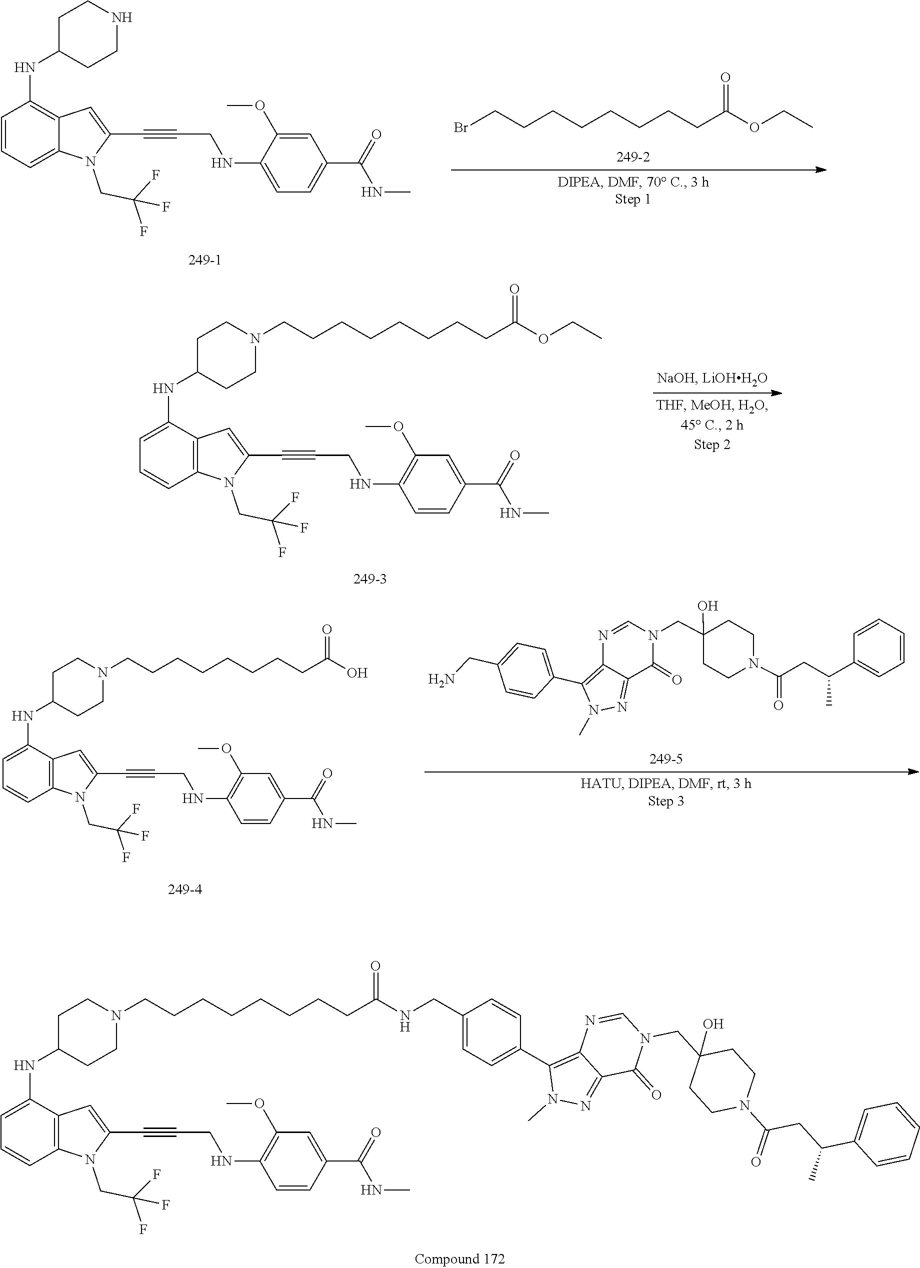 [SMILES: CNC(=O)c1ccc(NCC#Cc2cc3c(NC4CCNCC4)cccc3n2CC(F)(F)F)c(OC)c1 ; CCOC(=O)CCCCCCCCBr ; CCOC(=O)CCCCCCCCN1CCC(Nc2cccc3c2cc(C#CCNc2ccc(C(=O)NC)cc2OC)n3CC(F)(F)F)CC1 ; CNC(=O)c1ccc(NCC#Cc2cc3c(NC4CCN(CCCCCCCCC(=O)O)CC4)cccc3n2CC(F)(F)F)c(OC)c1 ; C[C@H](CC(=O)N1CCC(O)(Cn2cnc3c(-c4ccc(CN)cc4)n(C)nc3c2=O)CC1)c1ccccc1 ; CNC(=O)c1ccc(NCC#Cc2cc3c(NC4CCN(CCCCCCCCC(=O)NCc5ccc(-c6c7ncn(CC8(O)CCN(C(=O)C[C@@H](C)c9ccccc9)CC8)c(=O)c7nn6C)cc5)CC4)cccc3n2CC(F)(F)F)c(OC)c1]

### Step 1. Ethyl 9-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)nonanoate (249-3)

Into a 20 mL pressure-relief vial containing a well-stirred solution of 3-methoxy-N-methyl-4-((3-(4-(piperidin-4-ylamino)-1-(2,2,2-trifluoroethyl)-1H-indol-2-yl)prop-2-yn-1-yl)amino)benzamide (249-1, 250 mg, 0.49 mmol) in DMF (4 mL) were added ethyl 9-bromononanoate (249-2, 387 mg, 1.46 mmol) and DIPEA (1.70 mL, 9.74 mmol) at RT. The resultant mixture was heated at 70° C. for 3 h. Afterwards, water (50 mL) was added and extracted with EtOAc (2×25 mL). The combined organic layers were washed with water, brine, dried (Na₂SO₄) and concentrated under reduced pressure to obtain the crude material. Purification by a silica-gel (230-400 mesh size) flash column chromatography (10% DCM in Methanol) afforded the desired product Ethyl 9-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)nonanoate (249-3, 160 mg, 97% purity, 46% yield) as a brown solid. LCMS: (Method B) 698.1 (M+H)⁺, Rt. 3.38 min, 97.14% (Max);

### Step 2: 9-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)nonanoic acid (249-4)

Into a 20 mL pressure-relief vial containing a well-stirred solution of 9-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)nonanoate (249-3, 150 mg, 0.21 mmol) in MeOH (2 mL), THE (2 mL) and H₂O (2 mL), were added NaOH (86 mg, 2.15 mmol)) and LiOH (51.5 mg, 2.149 mmol) at room temperature. The resultant mixture was heated at 45° C. for 6 hours before allowed to attain room temperature and concentrated under reduced pressure. The residue was diluted with water (10 mL), neutralized to pH ˜7 and then extracted with DCM (2×10 mL). The combined organic layers were washed with brine solution, dried over anhydrous sodium sulphate and concentrated under reduced pressure to the afford crude material that was washed with MTBE to obtain 9-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)nonanoic acid (249-4, 130 mg, 92% purity, 83% yield). LCMS: (Method B) 670.1 (M+H)⁺, Rt. 2.26 min, 92.79% (Max)

### Step 3. (R)-4-((3-(4-((1-(9-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-9-oxononyl)piperidin-4-yl)amino)-1-(2,2,2-trifluoroethyl)-1H-indol-2-yl)prop-2-yn-1-yl)amino)-3-methoxy-N-methylbenzamide (Compound 172)

Into a 25 mL single-necked round-bottomed flask containing a well-stirred solution of 9-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)nonanoic acid (249-4, 65.1 mg, 0.10 mmol) in DMF (2 mL) were added HATU (73.9 mg, 0.19 mmol) and DIPEA (0.17 mL, 0.97 mmol). The reaction mixture was stirred at room temperature for 1 h. Subsequently, ((R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 50 mg, 0.01 mmol) was added and the reaction was allowed to stir for another 3 h. After completion, the mixture was concentrated under reduced pressure to afford the crude material that was purified by a reversed-phase preparatory HPLC [Column: KROMOSIL-C18 (250×21.2 mm) 5 μm; Mobile phase A: 10 mM Ammonium bicarbonate in water and Mobile phase B: Acetonitrile in THF; Flow Rate: 15 mL/min] to afford (R)-4-((3-(4-((1-(9-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-9-oxononyl)piperidin-4-yl)amino)-1-(2,2,2-trifluoroethyl)-1H-indol-2-yl)prop-2-yn-1-yl)amino)-3-methoxy-N-methylbenzamide (Compound 172, 17.3 mg, 99% purity, 15% yield)

¹H NMR (400 MHz, DMSO-d₆) δ=8.40 (t, J=5.9 Hz, 1H), 8.13-8.07 (m, 1H), 7.98 (d, J=10.1 Hz, 1H), 7.67 (d, J=8.3 Hz, 2H), 7.46-7.40 (m, 3H), 7.35 (d, J=1.8 Hz, 1H), 7.30-7.21 (m, 4H), 7.20-7.12 (m, 1H), 7.06 (s, 1H), 6.99 (t, J=8.0 Hz, 1H), 6.76 (d, J=8.3 Hz, 1H), 6.68 (d, J=8.6 Hz, 1H), 6.15 (d, J=8.0 Hz, 1H), 5.98 (t, J=6.2 Hz, 1H), 5.46 (d, J=8.0 Hz, 1H), 4.95-4.84 (m, 3H), 4.35 (d, J=5.8 Hz, 2H), 4.31 (d, J=6.1 Hz, 2H), 4.10 (s, 3H), 4.07-3.97 (m, 2H), 3.95-3.88 (m, 1H), 3.84 (s, 3H), 3.72-3.58 (m, 1H), 3.22-3.11 (m, 2H), 2.89-2.80 (m, 3H), 2.76 (d, J=4.5 Hz, 3H), 2.62-2.56 (m, 2H), 2.29-2.22 (m, 2H), 2.17 (t, J=7.4 Hz, 2H), 2.04-1.86 (m, 4H), 1.60-1.48 (m, 3H), 1.48-1.34 (m, 6H), 1.33-1.24 (m, 10H), 1.21 (d, J=7.0 Hz, 3H). LCMS: (Method A) 1166.4 (M+H)⁺, Rt. 1.76 min, 98.6% (Max); HPLC: Rt. 4.39 min, 97.89% (Max).

### Example 250. Synthesis of (R)-4-((3-(4-((1-(4-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-4-oxobutanoyl)piperidin-4-yl)amino)-1-(2,2,2-trifluoroethyl)-1H-indol-2-yl)prop-2-yn-1-yl)amino)-3-methoxy-N-methylbenzamide (Compound 173)

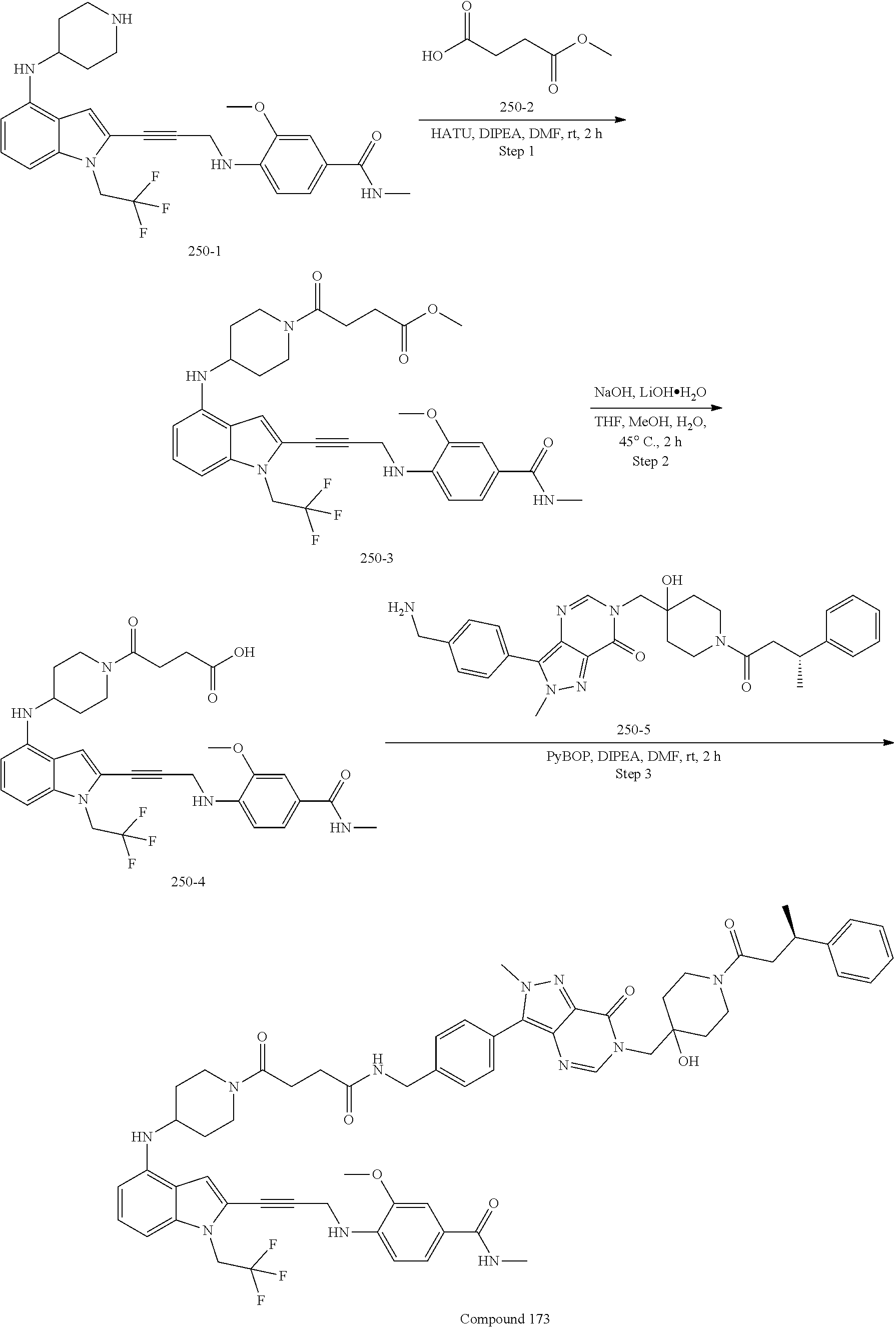

### Step 1: Methyl 4-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-4-oxobutanoate (250-3)

Into a 25 mL single-necked round-bottomed flask containing a well-stirred solution of 4-methoxy-4-oxobutanoic acid (250-2, 39.4 mg, 0.30 mmol) in anhydrous DMF (3 mL) were added DIPEA (0.52 mL, 2.98 mmol) and HATU (136 mg, 0.36 mmol) at room temperature. Subsequently, 3-methoxy-N-methyl-4-((3-(4-(piperidin-4-ylamino)-1-(2,2,2-trifluoroethyl)-1H-indol-2-yl)prop-2-yn-1-yl)amino)benzamide dihydrochloride (250-1, 175 mg, 0.30 mmol) was added and stirring was continued for another 2 hours. Afterwards, the mixture was concentrated under reduced pressure to afford the crude material. Purification by a flash silica-gel (230-400 mesh) column chromatography (0-20% MeOH/DCM) afforded methyl 4-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-4-oxobutanoate (250-3, 95 mg, 95% purity, 43% yield) as a brown solid.

LCMS: 628.0 (M+H)⁺, Rt. 2.58 min, 85.34% (Max);

### Step 2: 4-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)-4-oxobutanoic acid (250-4)

Into a 25 mL single-necked round-bottomed flask containing a well-stirred solution of methyl 4-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-4-oxobutanoate (250-3, 90 mg, 0.14 mmol) in MeOH (3 mL), THE (3 mL) and H₂O (3 mL) were added NaOH (57.4 mg, 1.43 mmol) LiOH·H₂O (60 mg, 1.43 mmol) at room temperature. The resultant mixture was heated at 45° C. for 2 hours before allowed to attain room temperature and concentrated under reduced pressure. The residue was dissolved in H₂O (10 mL) and neutralized to pH ˜6. The solid thus precipitated out was filtered and dried to afford 4-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-4-oxobutanoic acid (250-4, 85 mg, 86% purity, 83% yield) as a light brown solid. UPLC-MS: (Method C) 613.9 (M+H)⁺, Rt. 1.75 min, 86.40% (Max);

### Step 3: (R)-4-((3-(4-((1-(4-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-4-oxobutanoyl)piperidin-4-yl)amino)-1-(2,2,2-trifluoroethyl)-1H-indol-2-yl)prop-2-yn-1-yl)amino)-3-methoxy-N-methylbenzamide (Compound 173)

Into a 25 mL single-necked round-bottomed flask containing a well-stirred solution 4-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-4-oxobutanoic acid (250-4, 40 mg, 0.06 mmol) in anhydrous DMF (5 mL) were added DIPEA (0.10 mL, 0.65 mmol) and PyBOP (50.9 mg, 0.1 mmol)) at room temperature. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 36.9 mg, 0.07 mmol) was added and stirring was continued for another 2 hours. Afterwards, the mixture was concentrated under reduced pressure to afford the crude material that was purified by a reversed-phase preparatory HPLC [Column: X-Bridge C18 (19×150) mm, 5 μm; Mobile phase A: 10 mM Ammonium bicarbonate in water and Mobile phase B: Acetonitrile; Flow Rate: 12 mL/min] to afford (R)-4-((3-(4-((1-(4-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-4-oxobutanoyl)piperidin-4-yl)amino)-1-(2,2,2-trifluoroethyl)-1H-indol-2-yl)prop-2-yn-1-yl)amino)-3-methoxy-N-methylbenzamide (Compound 173, 19 mg, 99% purity, 26% yield) as an off-white solid.

¹H NMR (300 MHz, DMSO-d₆) δ=8.45 (t, J=5.7 Hz, 1H), 8.13-8.05 (m, 1H), 7.98 (d, J=7.7 Hz, 1H), 7.66 (d, J=8.2 Hz, 2H), 7.52-7.38 (m, 3H), 7.37-7.32 (m, 1H), 7.31-7.23 (m, 4H), 7.21-7.11 (m, 1H), 7.08-6.96 (m, 2H), 6.76 (d, J=8.3 Hz, 1H), 6.70 (d, J=7.9 Hz, 1H), 6.24 (d, J=7.8 Hz, 1H), 5.98 (t, J=6.4 Hz, 1H), 5.54 (d, J=8.6 Hz, 1H), 4.97-4.83 (m, 3H), 4.37 (d, J=5.7 Hz, 2H), 4.31 (d, J=6.3 Hz, 2H), 4.10 (s, 3H), 4.04-3.88 (m, 4H), 3.84 (s, 3H), 3.72-3.54 (m, 2H), 3.24-3.09 (m, 3H), 2.81-2.70 (m, 5H), 2.67-2.56 (m, 4H), 2.06-1.90 (m, 2H), 1.47-1.25 (m, 6H), 1.21 (d, J=6.8 Hz, 3H). Note: some protons are obscured by the solvent signals. LCMS: 1110.3 (M+H)⁺, Rt. 1.89 min, 99.38% (Max); HPLC: (Rt. 4.13 min, 98.25% (Max).

### Example 251. Synthesis of (R)-4-((3-(4-((1-(8-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-8-oxooctanoyl)piperidin-4-yl)amino)-1-(2,2,2-trifluoroethyl)-1H-indol-2-yl)prop-2-yn-1-yl)amino)-3-methoxy-N-methylbenzamide (Compound 174)

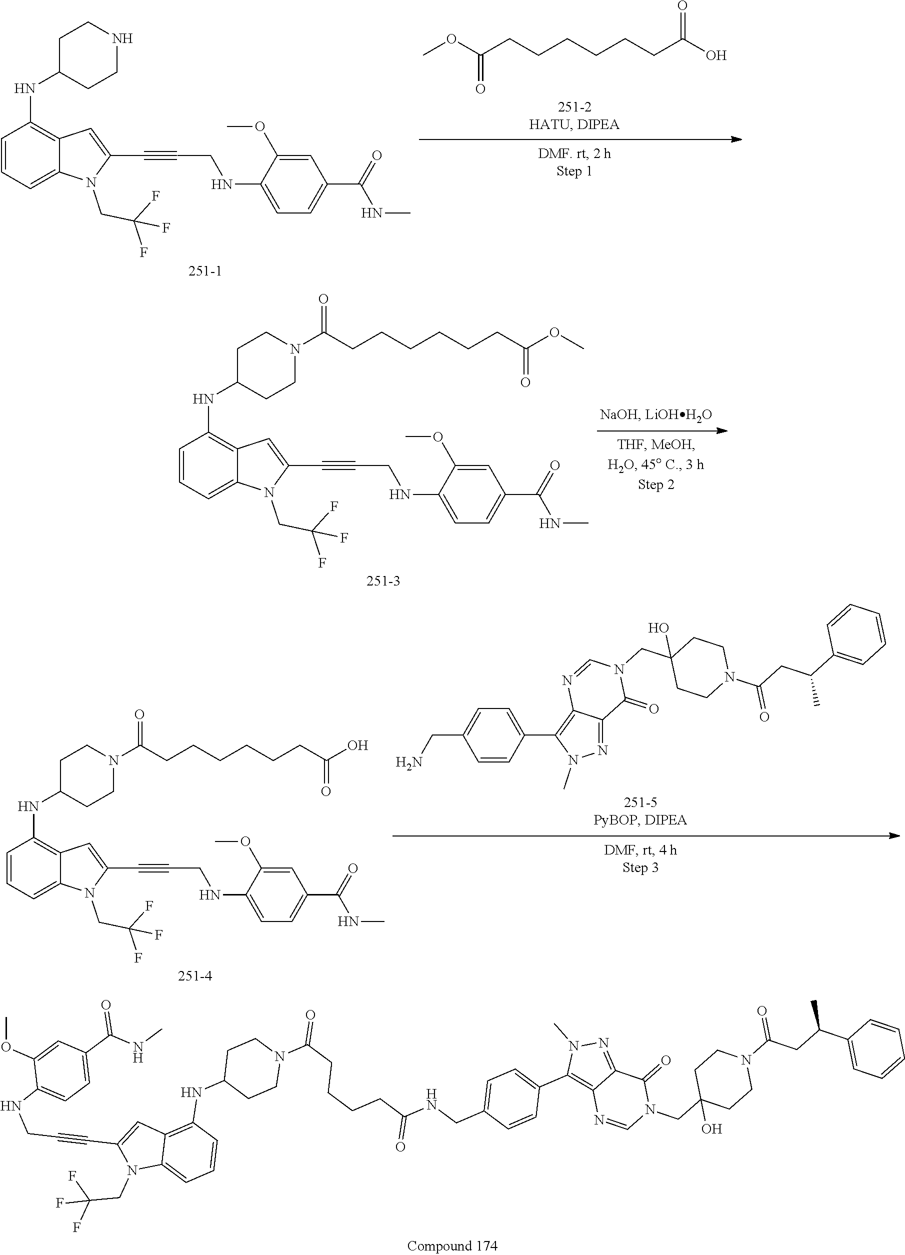 [SMILES: CNC(=O)c1ccc(NCC#Cc2cc3c(NC4CCNCC4)cccc3n2CC(F)(F)F)c(OC)c1 ; COC(=O)CCCCCCC(=O)O ; CNC(=O)c1ccc(NCC#Cc2cc3c(NC4CCN(C(=O)CCCCCCC(=O)OC)CC4)cccc3n2CC(F)(F)F)c(OC)c1 ; CNC(=O)c1ccc(NCC#Cc2cc3c(NC4CCN(C(=O)CCCCCCC(=O)O)CC4)cccc3n2CC(F)(F)F)c(OC)c1 ; C[C@H](CC(=O)N1CCC(O)(Cn2cnc3c(-c4ccc(CN)cc4)n(C)nc3c2=O)CC1)c1ccccc1 ; CNC(=O)c1ccc(NCC#Cc2cc3c(NC4CCN(C(=O)CCCCC(=O)NCc5ccc(-c6c7ncn(CC8(O)CCN(C(=O)C[C@@H](C)c9ccccc9)CC8)c(=O)c7nn6C)cc5)CC4)cccc3n2CC(F)(F)F)c(OC)c1]

### Step 1: Methyl 8-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-8-oxooctanoate (251-3)

Into a 25 mL single-necked round-bottomed flask containing a well-stirred solution of 8-methoxy-8-oxooctanoic acid (251-2, 49.1 mg, 0.26 mmol) in DMF (3 mL), were added HATU (165 mg, 0.43 mmol) and DIPEA (0.50 mL, 2.90 mmol). The reaction mixture was stirred at room temperature for 1 h. Subsequently, 3-methoxy-N-methyl-4-((3-(4-(piperidin-4-ylamino)-1-(2,2,2-trifluoroethyl)-1H-indol-2-yl)prop-2-yn-1-yl)amino)benzamide dihydrochloride (251-1, 170 mg, 0.29 mmol) was added and stirring was continued for another 2 h. After completion of reaction, the mixture was cooled to ambient temperature and diluted with water (10 mL). The aqueous layer was extracted with EtOAc (3×10 mL). The combined organic layers were washed with water (2×10 mL), brine (10 mL), dried (anhydrous Na₂SO₄), filtered and concentrated under reduced pressure to afford the crude material that was purified by a flash silica-gel (230-400 mesh) column chromatography (100% EtOAc) to afford methyl 8-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-8-oxooctanoate (251-3, 100 mg, 92% purity, 46% yield) as a light brown solid. LCMS: (Method B) 684.0 (M+H)⁺, Rt. 2.76 min, 92.53% (Max)

### Step 2. 8-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)-8-oxooctanoic acid (251-4)

Into a 25 mL single-necked round-bottomed flask containing a well-stirred solution of methyl 8-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-8-oxooctanoate (251-3, 100 mg, 0.14 mmol) in MeOH (2 mL), THE (2 mL) and H₂O (2 mL), were added NaOH (58.5 mg, 1.46 mmol) and LiOH·H₂O (35.0 mg, 1.46 mmol) at room temperature. The resultant mixture was heated at 45° C. for 3 hours before allowed to attain room temperature and concentrated under reduced pressure. The residue was dissolved in H₂O (10 mL) and neutralized to pH ˜7. The solid thus precipitated out was filtered and dried to afford 8-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-8-oxooctanoic acid (251-4, 70 mg, 96% purity, 69% yield) as a light brown solid. UPLC-MS: (Method C) 669.9 (M+H)⁺, Rt. 1.86 min, 92.53% (Max)

### Step 3. (R)-4-((3-(4-((1-(8-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-8-oxooctanoyl)piperidin-4-yl)amino)-1-(2,2,2-trifluoroethyl)-1H-indol-2-yl)prop-2-yn-1-yl)amino)-3-methoxy-N-methylbenzamide (Compound 174)

Into a 25 mL single-necked round-bottomed flask containing a well-stirred solution of 8-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-8-oxooctanoic acid (251-4, 70 mg, 0.10 mmol) in DMF (3 mL) were added DIPEA (0.183 mL, 1.045 mmol), PyBOP (82 mg, 0.16 mmol). The reaction mixture was stirred at room temperature for 1 h. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (251-5, 64 mg, 0.12 mmol) was added and the mixture was stirred for another 3 hours. After completion of reaction, the mixture was concentrated under reduced pressure to afford the crude material that was purified by a reversed-phase preparatory HPLC [Column: KROMOSIL-C18 (250×21.2 mm) 5 μm; Mobile phase A: 10 mM Ammonium bicarbonate in water and Mobile phase B: Acetonitrile; Flow rate: 15 mL/min] to afford (R)-4-((3-(4-((1-(8-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-8-oxooctanoyl)piperidin-4-yl)amino)-1-(2,2,2-trifluoroethyl)-1H-indol-2-yl)prop-2-yn-1-yl)amino)-3-methoxy-N-methylbenzamide (Compound 174, 48 mg, 96% purity, 38% yield) as a pale yellow solid.

¹H NMR (400 MHz, DMSO-d₆) δ=8.41 (t, J=6.0 Hz, 1H), 8.15-8.07 (m, 1H), 7.98 (d, J=10.0 Hz, 1H), 7.67 (d, J=8.5 Hz, 2H), 7.48-7.38 (m, 3H), 7.35 (d, J=2.0 Hz, 1H), 7.30-7.22 (m, 4H), 7.20-7.12 (m, 1H), 7.04 (s, 1H), 7.01 (t, J=8.0 Hz, 1H), 6.75 (d, J=8.5 Hz, 1H), 6.70 (d, J=8.5 Hz, 1H), 6.22 (d, J=8.0 Hz, 1H), 5.99 (t, J=6.3 Hz, 1H), 5.53 (d, J=8.0 Hz, 1H), 4.97-4.84 (m, 3H), 4.39-4.27 (m, 5H), 4.10 (s, 3H), 4.05-3.93 (m, 2H), 3.84 (s, 3H), 3.71-3.53 (m, 2H), 3.27-3.08 (m, 4H), 2.93-2.82 (m, 1H), 2.75 (d, J=4.5 Hz, 3H), 2.31 (t, J=7.3 Hz, 2H), 2.17 (t, J=7.3 Hz, 2H), 2.02-1.87 (m, 2H), 1.61-1.43 (m, 5H), 1.41-1.24 (m, 10H), 1.21 (d, J=7.0 Hz, 3H). LCMS: (Method A) 1167.5 (M+H)⁺, Rt. 2.01 min, 96.73% (Max); HPLC: (Method A) Rt. 4.31 min, 97.51% (Max)

### Example 252. Synthesis of (R)-4-((3-(4-((1-(6-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-6-oxohexanoyl)piperidin-4-yl)amino)-1-(2,2,2-trifluoroethyl)-1H-indol-2-yl)prop-2-yn-1-yl)amino)-3-methoxy-N-methylbenzamide (Compound 175)

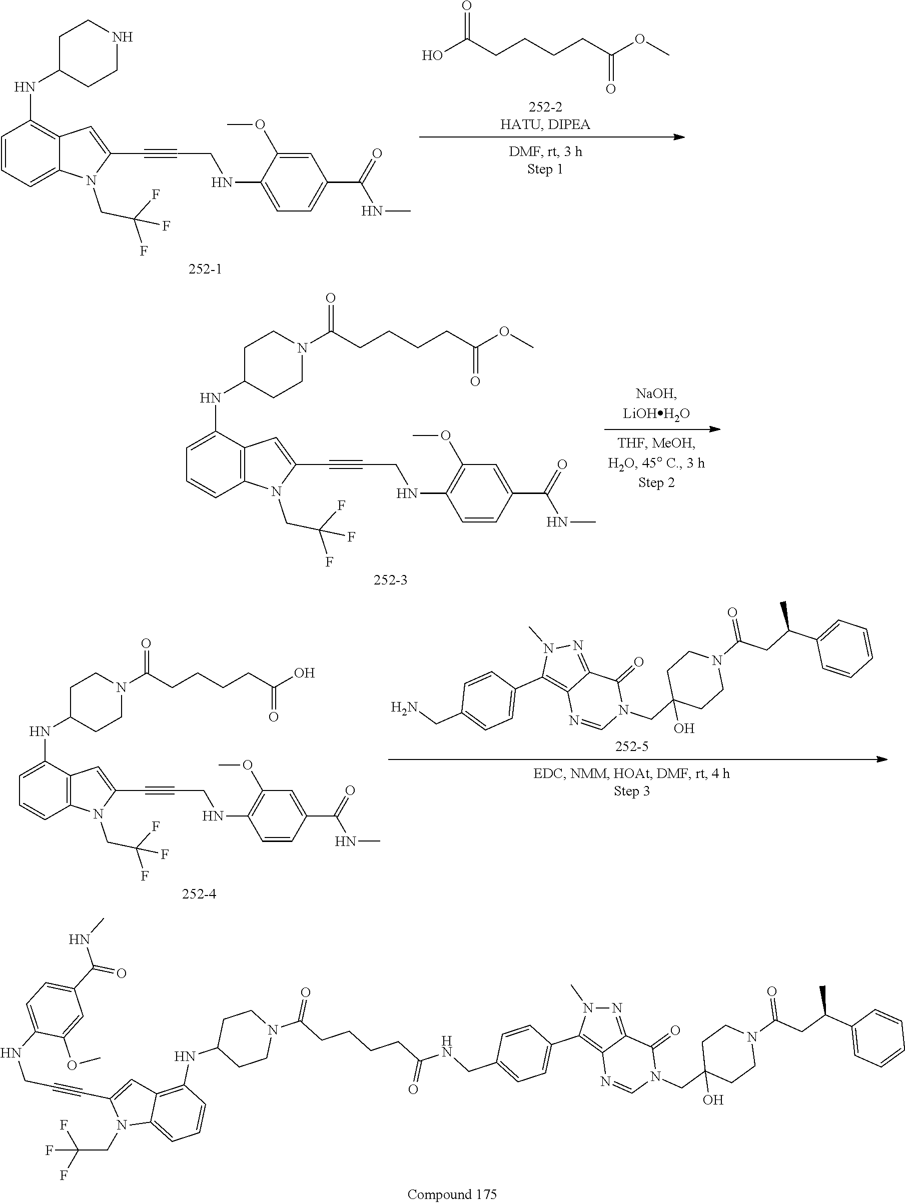 [SMILES: CNC(=O)c1ccc(NCC#Cc2cc3c(NC4CCNCC4)cccc3n2CC(F)(F)F)c(OC)c1 ; COC(=O)CCCCC(=O)O ; CNC(=O)c1ccc(NCC#Cc2cc3c(NC4CCN(C(=O)CCCCC(=O)OC)CC4)cccc3n2CC(F)(F)F)c(OC)c1 ; CNC(=O)c1ccc(NCC#Cc2cc3c(NC4CCN(C(=O)CCCCC(=O)O)CC4)cccc3n2CC(F)(F)F)c(OC)c1 ; C[C@H](CC(=O)N1CCC(O)(Cn2cnc3c(-c4ccc(CN)cc4)n(C)nc3c2=O)CC1)c1ccccc1 ; CNC(=O)c1ccc(NCC#Cc2cc3c(NC4CCN(C(=O)CCCCC(=O)NCc5ccc(-c6c7ncn(CC8(O)CCN(C(=O)C[C@@H](C)c9ccccc9)CC8)c(=O)c7nn6C)cc5)CC4)cccc3n2CC(F)(F)F)c(OC)c1]

### Step 1. Methyl 6-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-6-oxohexanoate (252-3)

Into a 25 mL single-necked round-bottomed flask containing a well-stirred solution of 6-methoxy-6-oxohexanoic acid (252-2, 46 mg, 0.29 mmol) in DMF (1.8 mL), were added HATU (181 mg, 0.48 mmol) and DIPEA (0.55 mL, 3.18 mmol). The reaction was stirred at RT for 1 h. Subsequently, 3-methoxy-N-methyl-4-((3-(4-(piperidin-4-ylamino)-1-(2,2,2-trifluoroethyl)-1H-indol-2-yl)prop-2-yn-1-yl)amino)benzamide hydrochloride (252-1, 175 mg, 0.32 mmol) was added and stirring was continued for another 2 h. Afterwards, the reaction mixture was concentrated under reduced pressure and washed with MTBE to obtain the crude methyl 6-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-6-oxohexanoate (252-3, 147 mg,) as brown solid, which as such was used for the next step. LCMS: (Method A) 656.3 (M+H)⁺, Rt. 2.00 min.

### Step 2. 6-(4-((2-(3-((2-Methoxy-4-(methylcarbamoyl)phenyl)amino)prop-1-yn-1-yl)-1-(2,2,2-trifluoroethyl)-1H-indol-4-yl)amino)piperidin-1-yl)-6-oxohexanoic acid (252-4)

Into a 25 mL single-necked round-bottomed flask containing a well-stirred solution of methyl 6-(4-((2-(3-((2-methoxy-4-(methylcarbamoyl)phenyl)amino)prop-1-yn-1-yl)-1-(2,2,2 trifluoroethyl)-1H-indol-4-yl)amino)piperidin-1-yl)-6-oxohexanoate (3, 140 mg, 0.21 mmol) in MeOH (3 mL), THE (3 mL) and H₂O (3 mL), were added NaOH (8.54 mg, 0.21 mmol) and LiOH·H₂O (8.96 mg, 0.21 mmol) at room temperature. The resultant mixture was heated at 45° C. for 3 hours before allowed to attain room temperature and concentrated under reduced pressure. The residue was dissolved in H₂O (10 mL) and neutralized to pH ˜7. The solid thus precipitated out was filtered and dried to afford 6-(4-((2-(3-((2-methoxy-4-(methylcarbamoyl)phenyl)amino)prop-1-yn-1-yl)-1-(2,2,2-trifluoroethyl)-1H-indol-4 yl)amino)piperidin-1-yl)-6-oxohexanoic acid (252-4, 67 mg, 84.3% purity, 42% yield) as a brown gummy solid. LCMS: (Method A) 642.2 (M+H)⁺, Rt. 1.77 min, 84.35% (Max);

### Step 3. (R)-4-((3-(4-((1-(6-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-6-oxohexanoyl)piperidin-4-yl)amino)-1-(2,2,2-trifluoroethyl)-1H-indol-2-yl)prop-2-yn-1-yl)amino)-3-methoxy-N-methylbenzamide (Compound 175)

Into a 25 mL single-necked round-bottomed flask containing a well-stirred solution of 6-(4-((2-(3-((2-methoxy-4-(methylcarbamoyl)phenyl)amino)prop-1-yn-1-yl)-1-(2,2,2-trifluoroethyl)-1H-indol-4-yl)amino)piperidin-1-yl)-6-oxohexanoic acid (252-4, 93 mg, 0.14 mmol) in DMF (1.5 mL) were added EDC (28 mg, 0.14 mmol), 1-hydroxy-7-azabenzotriazole (20 mg, 0.14 mmol) and N-methylmorpholine (15 mg, 0.14 mmol). The reaction mixture was stirred at room temperature for 1 h. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (75 mg, 0.14 mmol) was added and the reaction was allowed to stir for another 4 h. After completion of reaction, the mixture was concentrated under reduced pressure to afford the crude material that was purified by a reversed-phase preparatory HPLC [Column: KROMOSIL-C18 (250×21.2 mm) 5 μm; Mobile Phase A: 0.1% Ammonium bicarbonate in water and Mobile Phase B: Acetonitrile; Flow rate: 15 mL/min] to afford (R)-4-((3-(4-((1-(6-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-6-oxohexanoyl)piperidin-4-yl)amino)-1-(2,2,2-trifluoroethyl)-1H-indol-2-yl)prop-2-yn-1-yl)amino)-3-methoxy-N-methylbenzamide (Compound 175, 40.17 mg, 95.3% purity, 23% yield) as a pale yellow solid.

¹H NMR (400 MHz, DMSO-d₆) δ=8.43 (t, J=5.8 Hz, 1H), 8.16-8.07 (m, 1H), 7.97 (d, J=10.0 Hz, 1H), 7.66 (d, J=8.5 Hz, 2H), 7.47-7.38 (m, 3H), 7.35 (s, 1H), 7.31-7.21 (m, 4H), 7.20-7.12 (m, 1H), 7.05-6.96 (m, 2H), 6.75 (d, J=8.0 Hz, 1H), 6.70 (d, J=8.0 Hz, 1H), 6.22 (d, J=8.0 Hz, 1H), 5.99 (t, J=6.3 Hz, 1H), 5.53 (d, J=8.0 Hz, 1H), 4.96-4.83 (m, 3H), 4.40-4.26 (m, 5H), 4.10 (s, 3H), 4.06-3.87 (m, 4H), 3.84 (s, 3H), 3.71-3.54 (m, 2H), 3.24-3.09 (m, 3H), 2.93-2.81 (m, 1H), 2.75 (d, J=4.5 Hz, 3H), 2.20 (t, J=7.0 Hz, 2H), 2.05-1.87 (m, 3H), 1.64-1.47 (m, 5H), 1.42-1.24 (m, 6H), 1.21 (d, J=7.0 Hz, 3H). Note: a few aliphatic protons are obscured by the solvent signals. LCMS: (Method B) 1139.1 (M+H)⁺, Rt. 2.56 min, 94.15% (Max); HPLC: (Method A) Rt. 4.07 min, 93.93% (Max).

### Example 253. Synthesis of (R)—N-(2-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)-3-methoxy-4-((3-(4-((1-methylpiperidin-4-yl)amino)-1-(2,2,2-trifluoroethyl)-1H-indol-2-yl)prop-2-yn-1-yl)amino)benzamide (Compound 176)

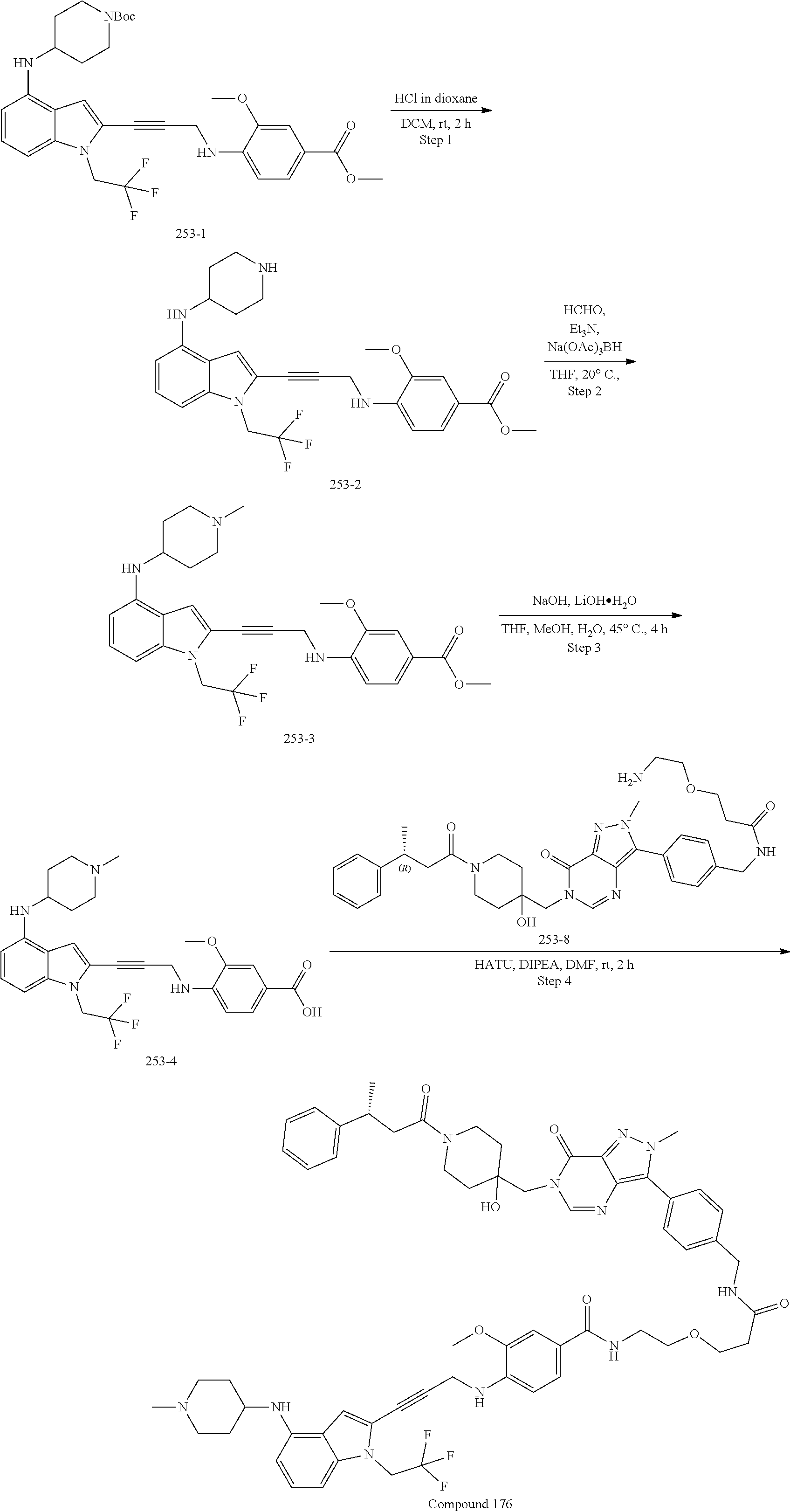

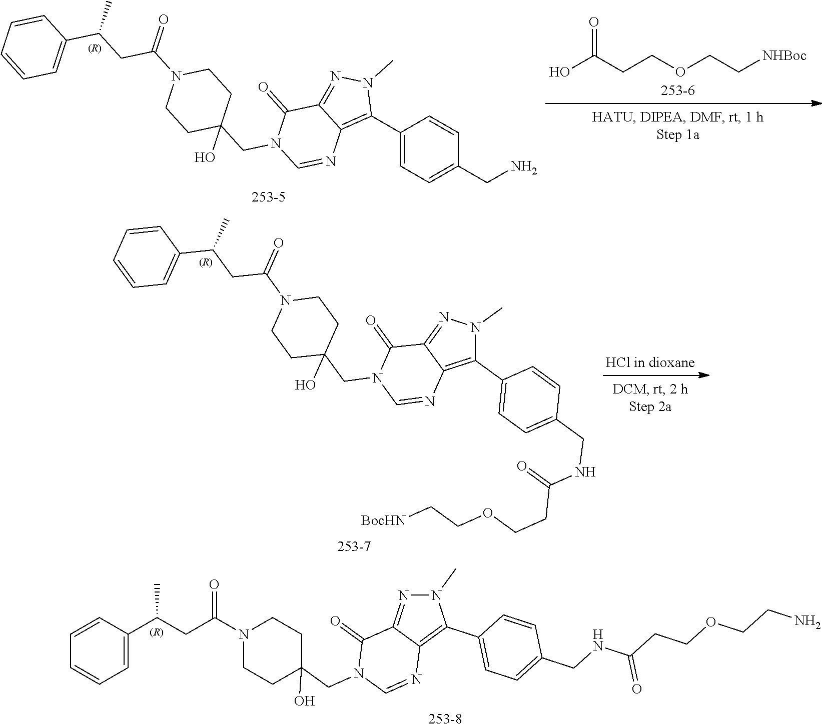

### Step 1: Methyl 3-methoxy-4-((3-(4-(piperidin-4-ylamino)-1-(2,2,2-trifluoroethyl)-1H-indol-2-yl)prop-2-yn-1-yl)amino)benzoate hydrochloride (253-2)

Into a 25 mL single-necked round-bottomed flask containing a well-stirred solution of tert-butyl 4-((2-(3-((2-methoxy-4-(methoxycarbonyl)phenyl)amino)prop-1-yn-1-yl)-1-(2,2,2-trifluoroethyl)-1H-indol-4-yl)amino)piperidine-1-carboxylate (253-1, 500 mg, 0.81 mmol) in dry DCM (10 mL) was added HCl in 4M Dioxane (5 mL, 20 mmol) at 25° C. The resulting mixture was stirred at room temperature for 2 hours and concentrated under reduced pressure. The crude material was triturated with MTBE (5 mL) to afford (methyl 3-methoxy-4-((3-(4-(piperidin-4-ylamino)-1-(2,2,2-trifluoroethyl)-1H-indol-2-yl)prop-2-yn-1-yl)amino)benzoate hydrochloride (253-2, 448 mg, 98% purity, 98% yield) as a light brown solid. LCMS: (Method A) 515.2 (M+H)⁺, Rt. 1.66 min, 98.47% (Max).

### Step 2: Methyl 3-methoxy-4-((3-(4-((1-methylpiperidin-4-yl)amino)-1-(2,2,2-trifluoroethyl)-1H-indol-2-yl)prop-2-yn-1-yl)amino)benzoate (253-3)

Into a 25 mL single-necked round-bottomed flask containing a well-stirred solution of methyl 3-methoxy-4-((3-(4-(piperidin-4-ylamino)-1-(2,2,2-trifluoroethyl)-1H-indol-2-yl)prop-2-yn-1-yl)amino)benzoate hydrochloride (253-2, 0.52 g, 0.94 mmol) in THE (3 mL) were added Et₃N (0.12 mL, 0.88 mmol) and aqueous HCHO (0.06 mL, 0.88 mmol) at 20° C. After 2 hours, NaBH(OAc)₃ (0.240 g, 1.13 mmol) was added and stirring was continued for another 1 hour. Thereafter, the mixture was poured into to ice-cold water (25 mL). The aqueous layer was extracted with EtOAc (2×25 mL). The combined organic layers were washed with water (2×25 mL), brine (25 mL), dried (anhydrous Na₂SO₄), filtered and concentrated under reduced pressure to afford the crude material. Purification by a flash silica-gel (230-400 mesh) column chromatography (5-10% MeOH/DCM) afforded methyl 3-methoxy-4-((3-(4-((1-methylpiperidin-4-yl)amino)-1-(2,2,2-trifluoroethyl)-1H-indol-2-yl)prop-2-yn-1-yl)amino)benzoate (3, 0.302 g, 98% purity, 61% yield). LCMS: (Method A) 529.3 (M+H)⁺, Rt. 1.67 min, 97.89% (Max).

### Step 3: 3-Methoxy-4-((3-(4-((1-methylpiperidin-4-yl)amino)-1-(2,2,2-trifluoroethyl)-1H-indol-2-yl)prop-2-yn-1-yl)amino)benzoic acid (253-4)

Into a 25 mL single-necked round-bottomed flask containing a well-stirred solution of methyl 3-methoxy-4-((3-(4-((1-methylpiperidin-4-yl)amino)-1-(2,2,2-trifluoroethyl)-1H-indol-2-yl)prop-2-yn-1-yl)amino)benzoate (253-3, 0.3 g, 0.57 mmol) in in MeOH (3 mL), THE (3 mL) and H₂O (3 mL) were added NaOH (0.227 g, 5.68 mmol) and LiOH·H₂O (0.238 g, 5.68 mmol) at room temperature. The resultant mixture was heated at 45° C. for 4 hours before allowed to attain room temperature and concentrated under reduced pressure. The residue was dissolved in H₂O (10 mL) and neutralized to pH ˜7. The solid thus precipitated out was filtered and dried to afford crude material. Purification by a flash silica-gel (230-400 mesh) column chromatography (5-10% MeOH/DCM) afforded 3-methoxy-4-((3-(4-((1-methylpiperidin-4-yl)amino)-1-(2,2,2-trifluoroethyl)-1H-indol-2-yl)prop-2-yn-1-yl)amino)benzoic acid (253-4, 0.15 g, 95% purity, 49% yield) as a pale yellow solid. LCMS: (Method B) 515.0 (M+H)⁺, Rt. 2.13 min, 94.81% (Max).

### Step 1a. tert-Butyl (R)-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)carbamate (253-7)

Into a 50 mL single-necked round-bottomed flask containing a well-stirred solution of 4 3-(2-((tert-butoxycarbonyl)amino)ethoxy)propanoic acid (253-6, 91 mg, 0.39 mmol) in DMF (2 mL) were added N-methylmorpholine (0.42 mL, 3.89 mmol), EDC·HCl (112 mg, 0.58 mmol) and HOAt (89 mg, 0.58 mmol) at 25° C. The resulting mixture was stirred at ambient temperature for 5 minutes. Subsequently, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 200 mg, 0.39 mmol) was added and stirring was continued for another 1 hour. Afterwards, the mixture was poured into to ice-cold water (20 mL). The aqueous layer was extracted with EtOAc (3×10 mL). The combined organic layers were washed with water (2×10 mL), brine (10 mL), dried (anhydrous Na₂SO₄), filtered and concentrated under reduced pressure to afford the crude material. Purification by a flash silica-gel (230-400 mesh) column chromatography (10% MeOH/DCM) afforded tert-butyl (R)-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)carbamate (7, 0.195 g, 99% purity, 68% yield) as an off-white solid. LCMS: (Method D) 730.4 (M+H)⁺, Rt. 1.78 min, 99.11% (Max).

### Step 2a. (R)-3-(2-aminoethoxy)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)propanamide hydrochloride (253-8)

Into a 25 mL single-necked round-bottomed flask containing a well-stirred solution of tert-butyl (R)-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)carbamate 253-(7, 0.197 g, 0.27 mmol) in DCM (3 mL) was added HCl in 4M Dioxane (1.70 mL, 6.75 mmol) at 25° C. The resulting mixture was stirred at room temperature for 2 hours and concentrated under reduced pressure. The crude material was triturated with MTBE (5 mL) to afford (R)-3-(2-aminoethoxy)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)propanamide hydrochloride (253-8, 0.22 g, 79% purity, 97% yield) as an off-white solid. LCMS: (Method B) 630.1 (M+H)⁺, Rt. 2.08 min, 79.26% (Max).

### Step 4: (R)—N-(2-(3-((4-(6-((4-Hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)-3-methoxy-4-((3-(4-((1-methylpiperidin-4-yl)amino)-1-(2,2,2-trifluoroethyl)-1H-indol-2-yl)prop-2-yn-1-yl)amino)benzamide (Compound 176)

Into a 25 mL single-necked round-bottomed flask containing a well-stirred solution 3-methoxy-4-((3-(4-((1-methylpiperidin-4-yl)amino)-1-(2,2,2-trifluoroethyl)-1H-indol-2-yl)prop-2-yn-1-yl)amino)benzoic acid (253-4, 0.147 g, 0.28 mmol) in anhydrous DMF (5 mL) were added N-methylmorpholine (0.5 mL, 4.28 mmol), EDC·HCl (0.11 g, 0.57 mmol) and HOAt (0.087 g, 0.57 mmol) at room temperature. Subsequently, (R)-3-(2-aminoethoxy)-N-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)propanamide hydrochloride (253-8, 0.19 g, 0.285 mmol) was added and stirring was continued for another 2 hours. Afterwards, the mixture was concentrated under reduced pressure to afford the crude material that was purified by a reversed-phase preparatory HPLC [Column: X-Bridge C8 (19.1×250) mm, 5 μm; Mobile phase A: 10 mM Ammonium bicarbonate in H₂O and Mobile phase B: MeCN] to afford (R)—N-(2-(3-((4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)amino)-3-oxopropoxy)ethyl)-3-methoxy-4-((3-(4-((1-methylpiperidin-4-yl)amino)-1-(2,2,2-trifluoroethyl)-1H-indol-2-yl)prop-2-yn-1-yl)amino)benzamide (Compound 176, 9.5 mg, 95% purity) as a light yellow solid.

¹H NMR (400 MHz, DMSO-d₆) δ=8.47 (t, J=5.9 Hz, 1H), 8.17 (t, J=5.3 Hz, 1H), 7.97 (d, J=10.1 Hz, 1H), 7.65 (d, J=8.3 Hz, 2H), 7.48-7.40 (m, 3H), 7.37 (d, J=1.8 Hz, 1H), 7.32-7.21 (m, 4H), 7.21-7.13 (m, 1H), 7.06 (s, 1H), 6.99 (t, J=8.1 Hz, 1H), 6.75 (d, J=8.3 Hz, 1H), 6.68 (d, J=8.4 Hz, 1H), 6.15 (d, J=7.8 Hz, 1H), 6.00 (t, J=6.4 Hz, 1H), 5.47 (d, J=7.8 Hz, 1H), 4.96-4.83 (m, 2H), 4.36 (br d, J=5.6 Hz, 2H), 4.31 (br d, J=6.1 Hz, 2H), 4.09 (s, 3H), 4.05-3.87 (m, 3H), 3.83 (s, 3H), 3.70 (t, J=6.4 Hz, 2H), 3.66-3.58 (m, 1H), 3.55-3.49 (m, 2H), 3.45-3.38 (m, 4H), 3.22-3.12 (m, 2H), 2.93-2.74 (m, 3H), 2.65-2.56 (m, 2H), 2.19 (s, 3H), 2.09-1.98 (m, 2H), 1.96-1.86 (m, 2H), 1.55-1.43 (m, 2H), 1.41-1.27 (m, 3H), 1.21 (d, J=7.0 Hz, 3H). Note: Some aliphatic protons are obscured by the solvent signals. LCMS: (Method A) 1127.4 (M+H)⁺, Rt. 1.64 min, 95.10% (Max); HPLC: (Method B) Rt. 5.68 min, 97.19% (Max).

### Example 254. Synthesis of 3-(2-(4-((R)-3-(3,6-dibromo-9H-carbazol-9-yl)-2-hydroxypropyl)piperazin-1-yl)ethoxy)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)propanamide (Compound 177)

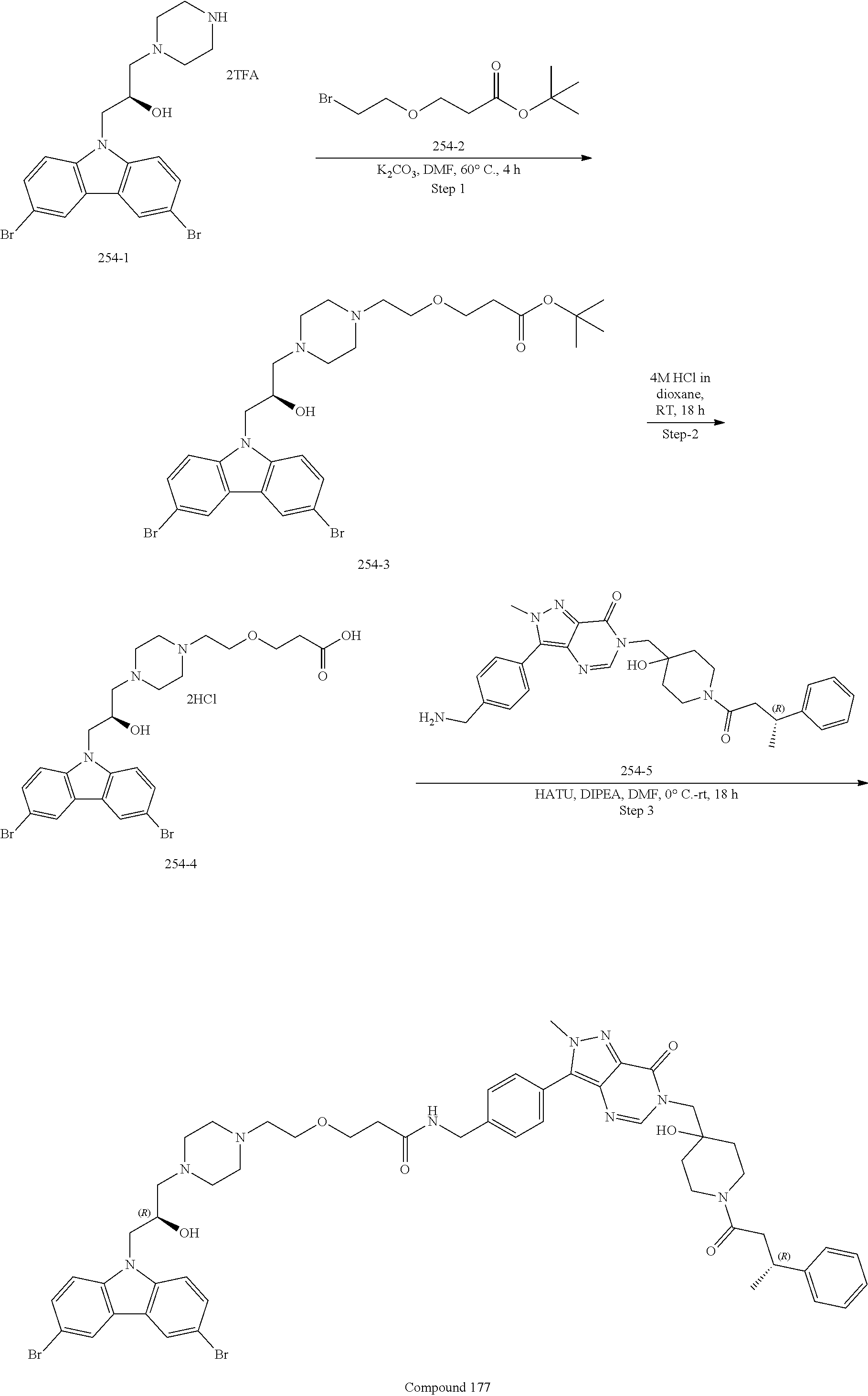

### Step 1: tert-butyl (R)-3-(2-(4-(3-(3,6-dibromo-9H-carbazol-9-yl)-2-hydroxypropyl)piperazin-1-yl)ethoxy)propanoate (254-3)

To a solution of (R)-1-(3,6-dibromo-9H-carbazol-9-yl)-3-(piperazin-1-yl)propan-2-ol bis(2,2,2-trifluoroacetate) (254-1, 0.25 g, 0.36 mmol) in DMF (2.5 mL), K₂CO₃ (0.248 g, 1.80 mmol) and tert-butyl 3-(2-bromoethoxy)propanoate (254-2, 0.137 g, 0.54 mmol) were added at RT. The resulting mixture was heated to 60° C. for 3.0 h. After completion (monitored by LCMS), the reaction was quenched with cold water (20 mL) and extracted with DCM (2×10 mL). The combined organic layer was dried over sodium sulphate and concentrated under vacuum to get the title compound (254-3, 0.21 g, 87% yield) as a pale-yellow gum. ¹H NMR (300 MHz, DMSO-d₆) δ=8.46 (d, J=1.5 Hz, 2H), 7.67-7.55 (m, 4H), 4.94 (d, J=5.0 Hz, 1H), 4.49-4.39 (m, 1H), 4.33-4.23 (m, 1H), 4.08-3.96 (m, 1H), 3.57 (t, J=6.1 Hz, 2H), 3.47 (t, J=5.7 Hz, 2H), 2.47-2.23 (m, 14H), 1.40 (s, 9H). LCMS: (Method C) 639.8 (M+H)⁺, Rt. 3.13 min, 91.07% (Max).

### Step 2: (R)-3-(2-(4-(3-(3,6-dibromo-9H-carbazol-9-yl)-2-hydroxypropyl)piperazin-1-yl)ethoxy)propanoic acid dihydrochloride (254-4)

To a stirred solution of tert-butyl (R)-3-(2-(4-(3-(3,6-dibromo-9H-carbazol-9-yl)-2-hydroxypropyl)piperazin-1-yl)ethoxy)propanoate (254-3, 0.2 g, 0.313 mmol) in 1,4-dioxane (5.0 mL) was added HCl (4 M in dioxane) (1.56 mL, 6.26 mmol) at RT and the resulting mixture was stirred at RT for 18 h. After completion (monitored by LCMS), the reaction mixture was concentrated under reduced pressure. The residue was triturated with MTBE, the solvent was decanted, and the resulting residue was dried to afford the title compound (254-4, 0.19 g, 86% yield) as an off-white solid. ¹H NMR (300 MHz, DMSO-d₆) δ=8.49 (d, J=1.9 Hz, 2H), 7.73 (d, J=8.8 Hz, 2H), 7.67-7.59 (m, 2H), 4.52-4.36 (m, 3H), 3.87-3.60 (m, 12H), 3.54-3.40 (m, 4H), 2.49-2.44 (m, 2H). LCMS: (Method B) 584.1 (M+H)⁺, Rt. 1.77 min, 92.8% (Max).

### Step 3: 3-(2-(4-((R)-3-(3,6-dibromo-9H-carbazol-9-yl)-2-hydroxypropyl)piperazin-1-yl)ethoxy)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)propanamide. (Compound 177)

To a solution of (R)-3-(2-(4-(3-(3,6-dibromo-9H-carbazol-9-yl)-2-hydroxypropyl)piperazin-1-yl)ethoxy)propanoic acid dihydrochloride (254-4, 0.15 g, 0.23 mmol), in DMF (2.5 mL), DIPEA (0.148 g, 1.14 mmol) and HATU (0.130 g, 0.34 mmol) were added at 0° C. After 10 minutes of stirring, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (254-5, 0.118 g, 0.23 mmol) was added and the reaction mixture was stirred for 6 h at RT. After completion (monitored by LCMS), the reaction was quenched with ice-water and extracted with DCM (2×10 mL). The combined organic extract was washed with brine (10 mL) and concentrated under reduced pressure. The crude residue was purified by reverse phase preparative HPLC (Purification method: X SELECT C18 (19×250) mm, 5 micron; Mobile phase A: 10 mM Ammonium bicarbonate and Mobile phase B: Acetonitrile, Flow rate=14 mL/minute) to get the title compound (52 mg, 20.38%, yield) as a white solid. ¹H NMR (400 MHz, DMSO-d₆) δ=8.50-8.42 (m, 3H), 7.95 (d, J=10.5 Hz, 1H), 7.66 (d, J=8.3 Hz, 2H), 7.63-7.55 (m, 4H), 7.45 (d, J=8.4 Hz, 2H), 7.31-7.23 (m, 4H), 7.20-7.12 (m, 1H), 4.93 (d, J=5.1 Hz, 1H), 4.85 (d, J=5.4 Hz, 1H), 4.46-4.36 (m, 3H), 4.30-4.20 (m, 1H), 4.10 (s, 3H), 4.02-3.84 (m, 4H), 3.68-3.58 (m, 3H), 3.51 (t, J=5.8 Hz, 2H), 3.23-3.14 (m, 2H), 2.93-2.83 (m, 1H), 2.62-2.56 (m, 2H), 2.45-2.21 (m, 14H), 1.56-1.25 (m, 4H), 1.21 (d, J=6.9 Hz, 3H). LCMS: (Method C) 1081.8 (M+H)⁺, Rt. 2.82 min, 97.99% (Max), HPLC: (Method A) Rt. 4.25 min, 96.44%.

### Example 258. Synthesis of 9-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)nonanamide (Compound 179)

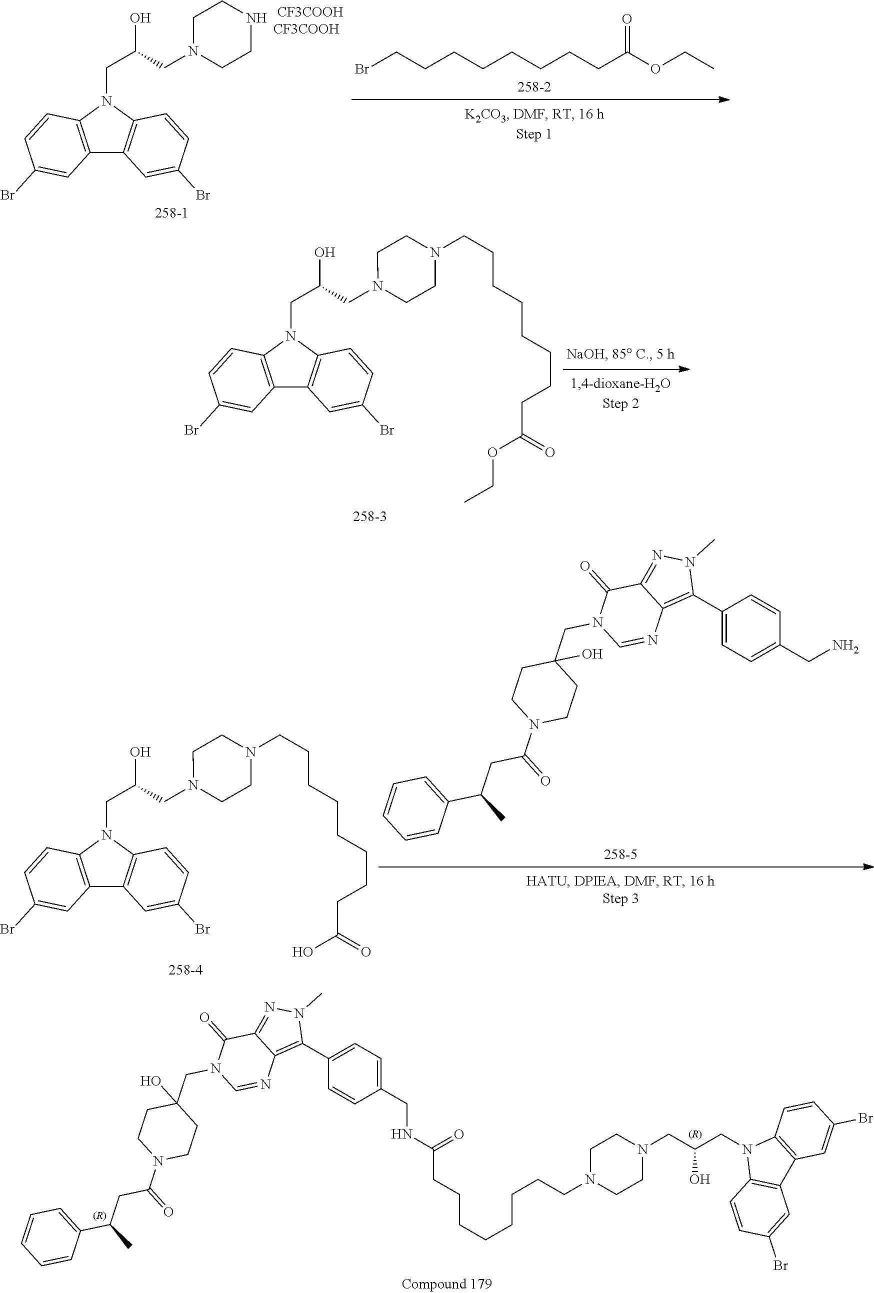

### Step 1: Ethyl (R)-9-(4-(3-(3,6-dibromo-9H-carbazol-9-yl)-2-hydroxypropyl)piperazin-1-yl)nonanoate (258-3)

To a stirred solution of (R)-1-(3,6-dibromo-9H-carbazol-9-yl)-3-(piperazin-1-yl)propan-2-ol bis(2,2,2-trifluoroacetate) (258-1, 0.15 g, 0.22 mmol) in DMF (0.75 mL), K₂CO₃ (0.15 g, 1.08 mmol) and ethyl 9-bromononanoate (258-2, 0.06 g, 0.24 mmol) were added, and the reaction mixture was stirred at RT for 16 h. After completion (monitored by TLC), the reaction mixture was diluted with water and extracted with EtOAc (2×5 mL). The combined organic extract was dried over anhydrous sodium sulphate, filtered, and concentrated under reduced pressure. The crude residue was purified by flash column chromatography using 230-400 mesh silica gel eluting with 5-10% MeOH in DCM as gradient to afford the title compound (258-3, 0.13 g, 88% yield) as pale brown oil. ¹H NMR (400 MHz, DMSO-d₆) δ=8.45 (d, J=1.6 Hz, 2H), 7.64-7.57 (m, 4H), 4.95 (s, 1H), 4.48-4.42 (m, 1H), 4.32-4.25 (m, 1H), 4.08-4.00 (m, 3H), 2.35-2.24 (m, 14H), 1.53-1.40 (m, 4H), 1.32-1.25 (m, 8H), 1.17 (t, J=9.6 Hz, 3H). LCMS: (Method C) 652.2 (M+H)⁺, Rt. 3.58 min, 95.34% (Max).

### Step 2: (R)-9-(4-(3-(3,6-dibromo-9H-carbazol-9-yl)-2-hydroxypropyl)piperazin-1-yl)nonanoic acid (258-4)

To a stirred solution of ethyl (R)-9-(4-(3-(3,6-dibromo-9H-carbazol-9-yl)-2-hydroxypropyl)piperazin-1-yl)nonanoate (258-3, 80 mg, 0.12 mmol) in 1,4-dioxane (1 mL), a solution of sodium hydroxide (15 mg, 0.37 mmol) in water (0.5 mL) was added and the reaction is stirred for 5 h at 80° C. After completion (monitored by TLC), the reaction mixture was concentrated under reduced pressure to remove dioxane. The resulting mixture was diluted with water and acidified using 1.5 N HCl and extracted using DCM (2×5 mL). The combined organic extract was dried over anhydrous sodium sulphate, filtered, and concentrated under reduced pressure to afford the title compound (258-4, 30 mg, 33% yield) as a brown solid. ¹H NMR (400 MHz, DMSO-d₆) δ=8.46 (d, J=1.6 Hz, 2H), 7.64-7.58 (m, 4H), 4.91 (br s, 1H), 4.46-4.44 (m, 1H), 4.31-4.30 (m, 1H), 4.02 (br s, 1H), 2.48-2.29 (m, 10H), 2.27-2.17 (m, 4H), 1.50-1.47 (m, 2H), 1.40-1.37 (m, 2H), 1.26-1.24 (m, 8H). LCMS: (Method A) 624.1 (M+H)⁺, Rt. 1.91 min, 95.84% (Max).

### Step 3: 9-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)nonanamide (Compound 179)

To a stirred solution of (R)-9-(4-(3-(3,6-dibromo-9H-carbazol-9-yl)-2-hydroxypropyl)piperazin-1-yl)nonanoic acid (258-4, 50 mg, 0.04 mmol) and (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 21 mg, 0.04 mmol) in DMF (0.5 mL) at 0° C., were added DIPEA (0.035 mL, 0.20 mmol) and HATU (23 mg, 0.06 mmol) and the reaction mixture was stirred at RT for 16 h. After completion (monitored by TLC), the reaction mixture was poured into ice cold-water (6 mL), and the precipitated solid was filtered and dried. The crude product was purified by reverse phase HPLC purification (Purification method: KROMOSIL C18 (19×250) mm, 5 micron; Mobile phase A: 10 mM Ammonium bicarbonate and Mobile phase B: MeCN, Flow rate=15 mL/minute) to afford the title compound (9 mg, 20% yield) as a white solid. ¹H NMR (400 MHz, DMSO-d₆) δ=8.45 (d, J=2.0 Hz, 2H), 8.41-8.37 (m, 1H), 7.98-7.96 (m, 1H), 7.68-7.56 (m, 6H), 7.45-7.42 (m, 2H), 7.27-7.24 (m, 4H), 7.18-7.15 (m, 1H), 4.87-4.86 (m, 2H), 4.43-4.29 (m, 4H), 4.10 (s, 3H), 4.04-3.90 (m, 4H), 3.75-3.59 (m, 1H), 3.53-3.38 (m, 1H), 3.28-3.06 (m, 2H), 2.91-2.88 (m, 2H), 2.45-2.12 (m, 14H), 1.65-1.55 (m, 3H), 1.48-1.38 (m, 3H), 1.27-1.19 (m, 13H). LCMS: (Method C) 1119.9 (M+H)⁺, Rt. 3.16 min, 98.17% (Max). HPLC: (Method C) Rt. 7.42 min, 99.77% (Max).

### Example 259. Synthesis of 9-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)nonanamide (Compound 180)

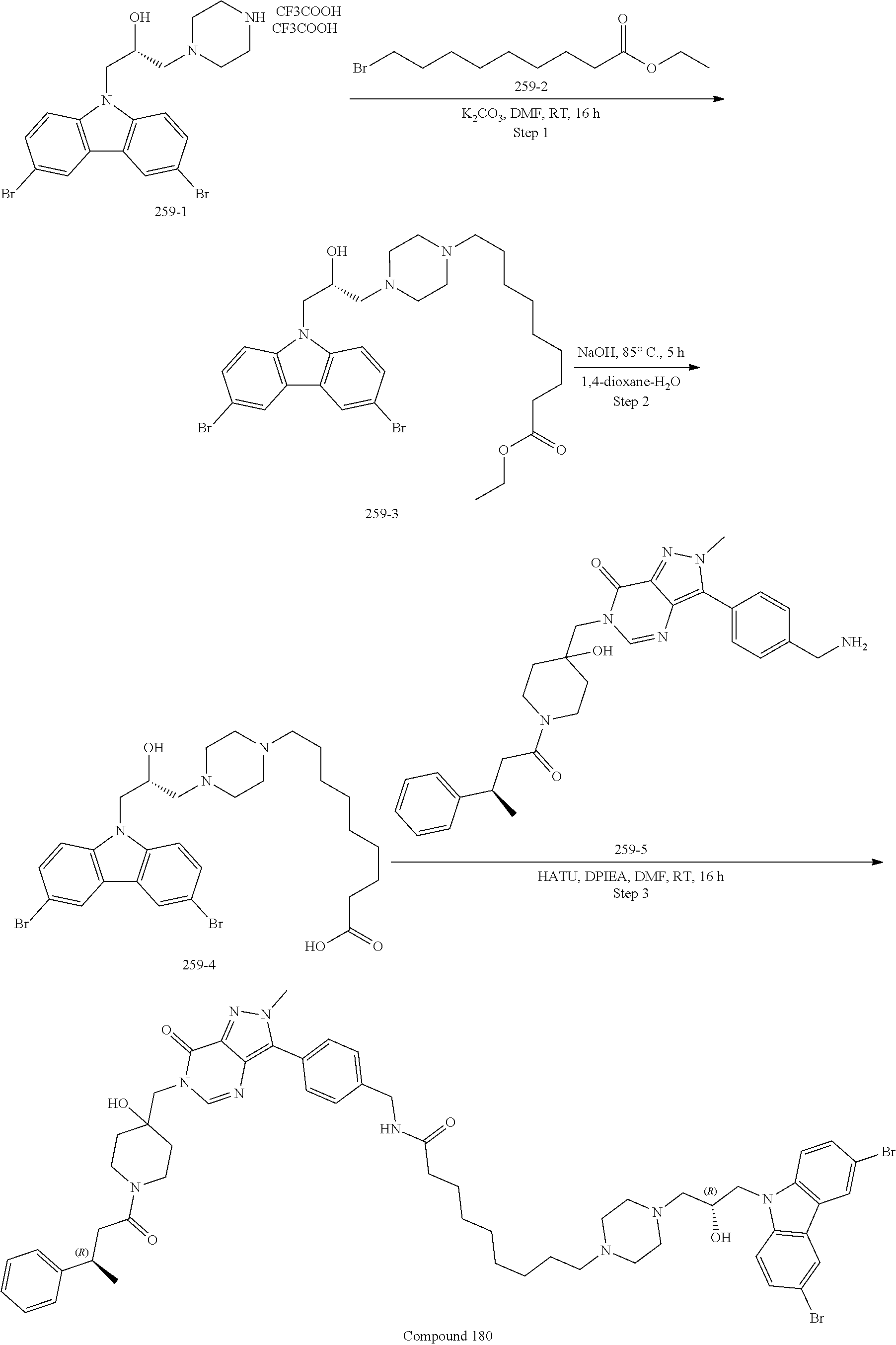

### Step 1: Ethyl (R)-9-(4-(3-(3,6-dibromo-9H-carbazol-9-yl)-2-hydroxypropyl)piperazin-1-yl)nonanoate (259-3)

To a stirred solution of (R)-1-(3,6-dibromo-9H-carbazol-9-yl)-3-(piperazin-1-yl)propan-2-ol bis(2,2,2-trifluoroacetate) (259-1, 0.15 g, 0.22 mmol) in DMF (0.75 mL), K₂CO₃ (0.15 g, 1.08 mmol) and ethyl 9-bromononanoate (259-2, 0.06 g, 0.24 mmol) were added, and the reaction mixture was stirred at RT for 16 h. After completion (monitored by TLC), the reaction mixture was diluted with water and extracted with EtOAc (2×5 mL). The combined organic extract was dried over anhydrous sodium sulphate, filtered, and concentrated under reduced pressure. The crude residue was purified by flash column chromatography using 230-400 mesh silica gel eluting with 5-10% MeOH in DCM as gradient to afford the title compound (259-3, 0.13 g, 88% yield) as pale brown oil. ¹H NMR (400 MHz, DMSO-d₆) δ=8.45 (d, J=1.6 Hz, 2H), 7.64-7.57 (m, 4H), 4.95 (s, 1H), 4.48-4.42 (m, 1H), 4.32-4.25 (m, 1H), 4.08-4.00 (m, 3H), 2.35-2.24 (m, 14H), 1.53-1.40 (m, 4H), 1.32-1.25 (m, 8H), 1.17 (t, J=9.6 Hz, 3H). LCMS: (Method C) 652.2 (M+H)⁺, Rt. 3.58 min, 95.34% (Max).

### Step 2: (R)-9-(4-(3-(3,6-dibromo-9H-carbazol-9-yl)-2-hydroxypropyl)piperazin-1-yl)nonanoic acid (259-4)

To a stirred solution of ethyl (R)-9-(4-(3-(3,6-dibromo-9H-carbazol-9-yl)-2-hydroxypropyl)piperazin-1-yl)nonanoate (259-3, 80 mg, 0.12 mmol) in 1,4-dioxane (1 mL), a solution of sodium hydroxide (15 mg, 0.37 mmol) in water (0.5 mL) was added and the reaction is stirred for 5 h at 80° C. After completion (monitored by TLC), the reaction mixture was concentrated under reduced pressure to remove dioxane. The resulting mixture was diluted with water and acidified using 1.5 N HCl and extracted using DCM (2×5 mL). The combined organic extract was dried over anhydrous sodium sulphate, filtered, and concentrated under reduced pressure to afford the title compound (259-4, 30 mg, 33% yield) as a brown solid. ¹H NMR (400 MHz, DMSO-d₆) δ=8.46 (d, J=1.6 Hz, 2H), 7.64-7.58 (m, 4H), 4.91 (br s, 1H), 4.46-4.44 (m, 1H), 4.31-4.30 (m, 1H), 4.02 (br s, 1H), 2.48-2.29 (m, 10H), 2.27-2.17 (m, 4H), 1.50-1.47 (m, 2H), 1.40-1.37 (m, 2H), 1.26-1.24 (m, 8H). LCMS: (Method A) 624.1 (M+H)⁺, Rt. 1.91 min, 95.84% (Max).

### Step 3: 9-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)nonanamide (Compound 180)

To a stirred solution of (R)-9-(4-(3-(3,6-dibromo-9H-carbazol-9-yl)-2-hydroxypropyl)piperazin-1-yl)nonanoic acid (259-4, 50 mg, 0.04 mmol) and (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (259-5, 21 mg, 0.04 mmol) in DMF (0.5 mL) at 0° C., were added DIPEA (0.035 mL, 0.20 mmol) and HATU (23 mg, 0.06 mmol) and the reaction mixture was stirred at RT for 16 h. After completion (monitored by TLC), the reaction mixture was poured into ice cold-water (6 mL), and the precipitated solid was filtered and dried. The crude product was purified by reverse phase HPLC purification (Purification method: KROMOSIL C18 (19×250) mm, 5 micron; Mobile phase A: 10 mM Ammonium bicarbonate and Mobile phase B: MeCN, Flow rate=15 mL/minute) to afford the title compound (9 mg, 20% yield) as a white solid. ¹H NMR (400 MHz, DMSO-d₆) δ=8.45 (d, J=2.0 Hz, 2H), 8.41-8.37 (m, 1H), 7.98-7.96 (m, 1H), 7.68-7.56 (m, 6H), 7.45-7.42 (m, 2H), 7.27-7.24 (m, 4H), 7.18-7.15 (m, 1H), 4.87-4.86 (m, 2H), 4.43-4.29 (m, 4H), 4.10 (s, 3H), 4.04-3.90 (m, 4H), 3.75-3.59 (m, 1H), 3.53-3.38 (m, 1H), 3.28-3.06 (m, 2H), 2.91-2.88 (m, 2H), 2.45-2.12 (m, 14H), 1.65-1.55 (m, 3H), 1.48-1.38 (m, 3H), 1.27-1.19 (m, 13H). LCMS: (Method C) 1119.9 (M+H)⁺, Rt. 3.16 min, 98.17% (Max). HPLC: (Method C) Rt. 7.42 min, 99.77% (Max).

### Example 260. Synthesis of 6-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)hexanamide (Compound 181)

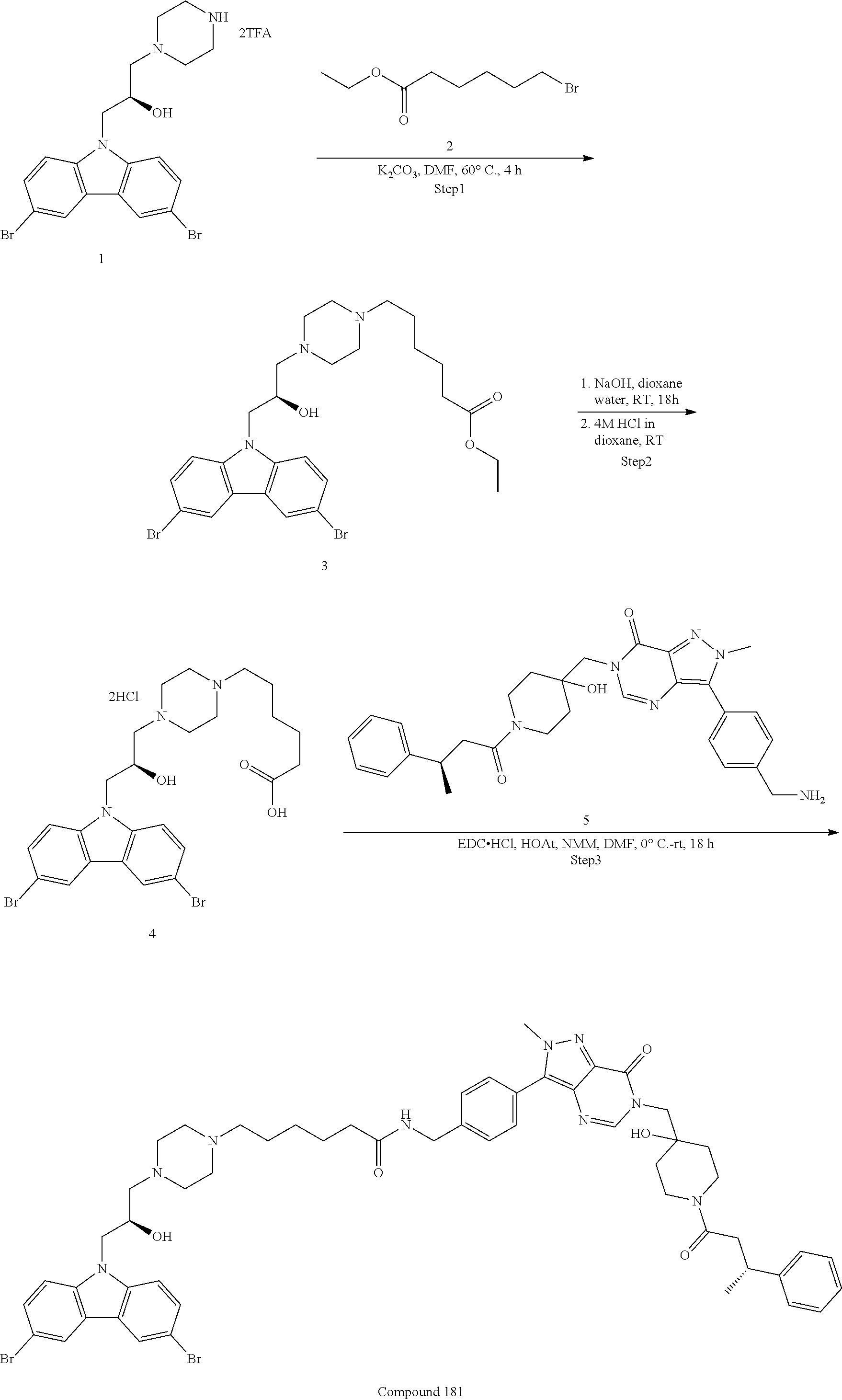 [SMILES: O[C@H](CN1CCNCC1)Cn1c2ccc(Br)cc2c2cc(Br)ccc21 ; CCOC(=O)CCCCCBr ; CCOC(=O)CCCCCN1CCN(C[C@@H](O)Cn2c3ccc(Br)cc3c3cc(Br)ccc32)CC1 ; O=C(O)CCCCCN1CCN(C[C@@H](O)Cn2c3ccc(Br)cc3c3cc(Br)ccc32)CC1 ; C[C@H](CC(=O)N1CCC(O)(Cn2cnc3c(-c4ccc(CN)cc4)n(C)nc3c2=O)CC1)c1ccccc1 ; C[C@H](CC(=O)N1CCC(O)(Cn2cnc3c(-c4ccc(CNC(=O)CCCCCN5CCN(C[C@@H](O)Cn6c7ccc(Br)cc7c7cc(Br)ccc76)CC5)cc4)n(C)nc3c2=O)CC1)c1ccccc1]

### Step 1: ethyl (R)-6-(4-(3-(3,6-dibromo-9H-carbazol-9-yl)-2-hydroxypropyl)piperazin-1-yl)hexanoate (260-3)

To stirred solution of (R)-1-(3,6-dibromo-9H-carbazol-9-yl)-3-(piperazin-1-yl)propan-2-ol bis(2,2,2-trifluoroacetate) (260-1, 0.250 g, 0.36 mmol) in DMF (2.5 mL), potassium carbonate (0.075 g, 0.539 mmol) and ethyl 6-bromohexanoate (260-2, 0.104 g, 0.47 mmol) were added and the reaction mixture heated at 60° C. for 3 h. After completion (monitored by LCMS), the reaction mixture was quenched with cold water (10 mL), extracted with DCM (2×10 mL). The combined organic layer was washed with brine (10 mL), dried over sodium sulphate, and concentrated to get the title compound (260-3, 0.15 g, 28% Yield) as a light brown gum. ¹H NMR (300 MHz, DMSO-d₆) δ=8.46 (d, J=1.3 Hz, 2H), 7.67-7.56 (m, 4H), 4.94 (d, J=4.8 Hz, 1H), 4.50-4.38 (m, 1H), 4.34-4.23 (m, 1H), 4.10-3.96 (m, 3H), 2.46-2.15 (m, 14H), 1.59-1.46 (m, 2H), 1.46-1.34 (m, 2H), 1.33-1.22 (m, 2H), 1.18 (t, J=7.1 Hz, 3H). LCMS: (Method C) 609.9 (M+H)⁺, Rt. 3.29 min, 76.56% (Max).

### Step 2: (R)-3-(2-(4-(3-(3,6-dibromo-9H-carbazol-9-yl)-2-hydroxypropyl)piperazin-1-yl)ethoxy)propanoic acid dihydrochloride (260-4)

To a stirred solution of ethyl (R)-6-(4-(3-(3,6-dibromo-9H-carbazol-9-yl)-2-hydroxypropyl)piperazin-1-yl)hexanoate (260-3, 0.125 g, 0.20 mmol) in 1,4-dioxane (1 mL), a solution of NaOH (25 mg, 0.61 mmol) in water (0.5 mL) was added and the resulting reaction mixture was stirred at RT overnight. After completion (monitored by LCMS), the reaction mixture was concentrated under vacuum. The residue was dissolved in dioxane, a solution of 4 M HCl in dioxane (1.0 mL) was added, and the mixture was stirred for 15 min at RT. The reaction mixture was concentrated under vacuum to get the title compound (260-4, 0.134 g, 100% yield) as an off-white solid. ¹H NMR (400 MHz, DMSO-d₆) δ=12.50-10.76 (m, 2H), 8.48 (d, J=2.0 Hz, 2H), 7.73 (d, J=8.5 Hz, 2H), 7.61 (dd, J=2.0, 9.0 Hz, 2H), 4.43 (br s, 3H), 3.82-3.60 (m, 4H), 3.54-3.49 (m, 3H), 3.30-3.01 (m, 5H), 2.27-2.17 (m, 2H), 1.76-1.63 (m, 2H), 1.58-1.45 (m, 2H), 1.38-1.26 (m, 2H). LCMS: (Method A) 582.0 (M+H)⁺, Rt. 1.64 min, 97.8% (Max).

### Step 3: 6-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)hexanamide (Compound 181)

To a stirred solution of (R)-6-(4-(3-(3,6-dibromo-9H-carbazol-9-yl)-2-hydroxypropyl)piperazin-1-yl)hexanoic acid dihydrochloride (260-4, 0.134 g, 0.20 mmol) in DMF (3 mL), EDC·HCl (59 mg, 0.31 mmol), 1-hydroxy-7-azabenzotriazole (42 mg, 0.31 mmol) and N-methyl morpholine (0.113 mL, 1.02 mmol) were added at 0° C. The resulting reaction mixture was stirred for 15 min at 0° C., then (R)-6-(4-(3-(3,6-dibromo-9H-carbazol-9-yl)-2-hydroxypropyl)piperazin-1-yl)hexanoic acid dihydrochloride (260-5, 0.134 g, 0.20 mmol) was added at 0° C. and the reaction mixture stirred for 18 h at RT. The reaction was monitored by LCMS. After completion, the reaction mixture was diluted with ice-water (10 mL) and extracted with DCM (2×10 mL). The combined organic layer was washed with brine (10 mL), dried over sodium sulphate, and concentrated under reduced pressure. The crude residue was purified by reverse phase preparative HPLC (Purification method: Xbridge C18 (19×250) mm, 5 micron; Mobile phase A: 10 mM Ammonium bicarbonate and Mobile phase B: Acetonitrile, Flow rate=12 mL/minute) to get the title compound (40.22 mg 17% yield), as a white solid. ¹H NMR (400 MHz, DMSO-d₆) δ=8.45 (d, J=1.8 Hz, 2H), 8.40 (t, J=5.9 Hz, 1H), 7.97 (d, J=10.1 Hz, 1H), 7.68-7.56 (m, 6H), 7.44 (d, J=8.4 Hz, 2H), 7.31-7.21 (m, 4H), 7.19-7.13 (m, 1H), 5.00-4.92 (m, 1H), 4.86 (d, J=4.9 Hz, 1H), 4.48-4.41 (m, 1H), 4.36 (d, J=5.8 Hz, 2H), 4.32-4.24 (m, 1H), 4.11 (s, 3H), 4.08-3.85 (m, 5H), 3.71-3.60 (m, 1H), 3.24-3.13 (m, 2H), 2.94-2.84 (m, 1H), 2.66-2.55 (m, 3H), 2.42-2.21 (m, 10H), 2.18 (t, J=7.4 Hz, 2H), 1.62-1.54 (m, 2H), 1.50-1.34 (m, 4H), 1.34-1.24 (m, 4H), 1.21 (d, J=7.0 Hz, 3H). LCMS: (Method C) 1077.9, (M+H)⁺, Rt. 2.94 min, 97.15% (Max). HPLC: (Method B) Rt. 6.53 min, 92.38%.

### Example 261. Synthesis of 5-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)pentanamide (Compound 182)

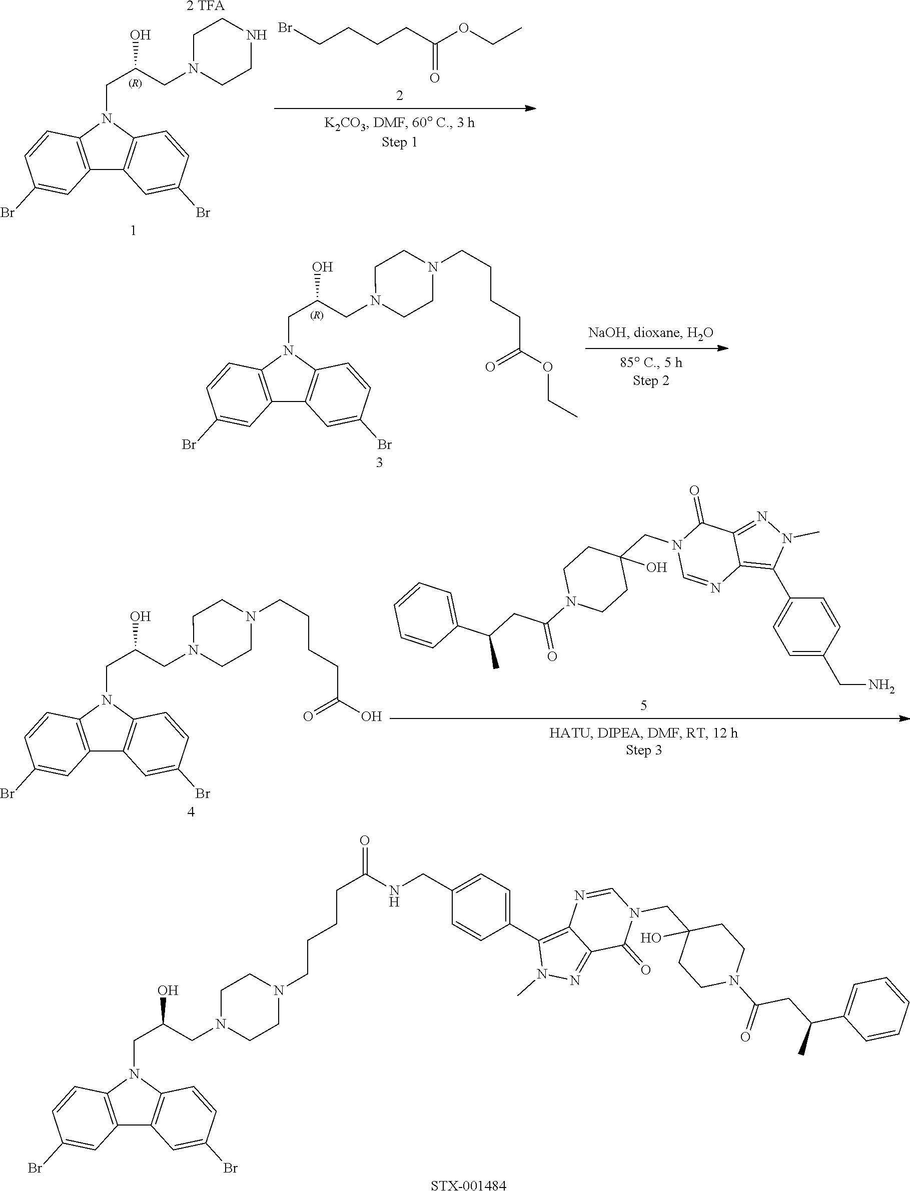

### Step 1: (R)-5-(4-(3-(3,6-dibromo-9H-carbazol-9-yl)-2-hydroxypropyl)piperazin-1-yl)pentanoate (261-3)

To a stirred solution of (R)-1-(3,6-dibromo-9H-carbazol-9-yl)-3-(piperazin-1-yl)propan-2-ol bis(2,2,2-trifluoroacetate) (261-1, 0.2 g, 0.28 mmol) in DMF (1 mL), K₂CO₃ (0.2 g, 1.4 mmol) and ethyl 5-bromopentanoate (261-2, 70 mg, 0.34 mmol) were added at RT, and the reaction mixture was stirred at 60° C. for 3 h. After completion (monitored by TLC), the reaction mixture was diluted with water and extracted with DCM (3×10 mL). The combined organic extract was dried over anhydrous sodium sulphate, filtered, and concentrated under reduced pressure. The crude residue was purified by flash column chromatography using 230-400 mesh silica gel eluting with 3-4% MeOH in DCM as gradient to afford the title compound (261-3, 50 mg, 28% yield) as gummy mass. ¹H NMR (400 MHz, CHLOROFORM-d) δ=8.16 (d, J=1.6 Hz, 2H), 7.58 (dd, J=2.0, 8.8 Hz, 2H), 7.40 (d, J=8.8 Hz, 2H), 4.40-4.27 (m, 2H), 4.19-4.11 (m, 3H), 2.68-2.47 (m, 2H), 2.42-2.28 (m, 12H), 1.67-1.51 (m, 4H), 1.34-1.10 (m, 3H). LCMS: (Method B) 596.0 (M+H)⁺, Rt. 2.28 min, 97.24% (Max).

### Step 2: (R)-5-(4-(3-(3,6-dibromo-9H-carbazol-9-yl)-2-hydroxypropyl)piperazin-1-yl)pentanoic acid dihydrochloride (261-4)

To a stirred solution of ethyl (R)-5-(4-(3-(3,6-dibromo-9H-carbazol-9-yl)-2-hydroxypropyl)piperazin-1-yl)pentanoate (261-3, 0.1 g, 0.17 mmol) in 1,4-dioxane (1 mL), a solution of sodium hydroxide (34 mg, 0.84 mmol) in water (0.5 mL) was added and the reaction was stirred for 16 h at RT. After completion (monitored by TLC), the reaction mixture was concentrated under reduced pressure to remove dioxane. The reaction mixture was diluted with water, acidified using 4 M HCl in dioxane and concentrated under reduced pressure to get the title compound (261-4, 90 mg, 80% yield) as a brown solid. ¹H NMR (400 MHz, DMSO-d₆) δ=11.90 (s, 1H), 8.47 (d, J=2.4 Hz, 2H), 7.73 (d, J=11.6 Hz, 2H), 7.62-7.59 (m, 2H), 4.46-4.38 (m, 3H), 3.63-3.04 (m, 12H), 2.30-2.18 (m, 2H), 1.79-1.69 (m, 2H), 1.52-1.48 (m, 2H). LCMS: (Method B) 568.1 (M+H), Rt. 1.85 min, 95.72% (Max).

### Step 3: 5-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)pentanamide, Compound 182

To a stirred solution of (R)-5-(4-(3-(3,6-dibromo-9H-carbazol-9-yl)-2-hydroxypropyl)piperazin-1-yl)pentanoic acid dihydrochloride (261-4, 95 mg, 0.15 mmol) in DMF (1.0 mL), DIPEA (0.13 ml, 0.74 mmol) and HATU (85 mg, 0.22 mmol) were added at 0° C. Then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (261-5, 76 mg, 0.15 mmol) was added at RT and the reaction mixture was stirred for 16 h. After completion (monitored by TLC), the reaction mixture was poured into ice-cold water (6 mL), the precipitated solid was filtered and dried. The crude product was purified by reverse phase HPLC purification (Purification method: Mobile phase A: 10 mM Ammonium bicarbonate and Mobile phase B: MeCN) to afford the title compound (20 mg, 12% yield) as an off-white solid. ¹H-NMR (400 MHz, DMSO-d₆) δ=8.45 (d, J=2.4 Hz, 2H), 8.41-8.38 (m, 1H), 7.98-7.96 (m, 1H), 7.68-7.56 (m, 6H), 7.45-7.42 (m, 2H), 7.18-7.13 (m, 4H), 7.27-7.24 (m, 1H), 4.97-4.86 (m, 2H), 4.46-4.24 (m, 4H), 4.10 (s, 3H), 3.99-3.89 (m, 4H), 3.72-3.59 (m, 1H), 3.25-3.12 (m, 2H), 2.87-2.79 (m, 1H), 2.64-2.51 (m, 1H), 2.42-2.14 (m, 15H), 1.55-1.53 (m, 2H), 1.46-1.24 (m, 6H) 1.24-1.19 (m, 3H). LCMS: (Method C) 1063.9 (M+H)⁺, Rt. 2.89 min, 97.90% (Max). HPLC: (Method A) Rt. 4.33 min, 98.79% (Max).

### Example 262. Synthesis of 4-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)butanamide (Compound 183)

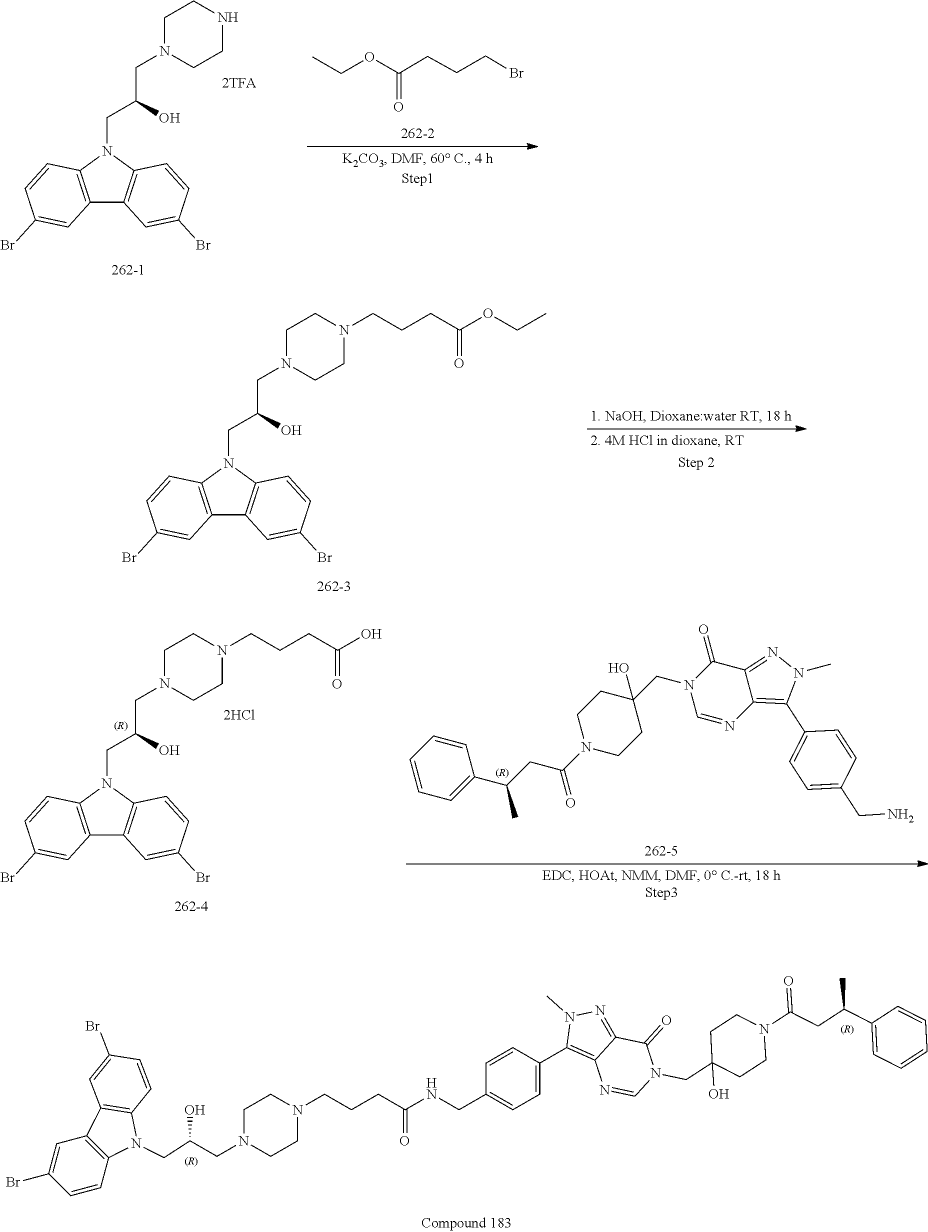

### Step 1: ethyl (ethyl (R)-4-(4-(3-(3,6-dibromo-9H-carbazol-9-yl)-2-hydroxypropyl)piperazin-1-yl)butanoate (262-3)

To stirred solution of (R)-1-(3,6-dibromo-9H-carbazol-9-yl)-3-(piperazin-1-yl)propan-2-ol bis(2,2,2-trifluoroacetate) (262-1, 0.250 g, 0.36 mmol) in DMF (2.5 mL) at RT, potassium carbonate (75 mg, 0.54 mmol) and ethyl 4-bromobutanoate (262-2, 91 mg, 0.47 mmol) was added and the reaction mixture heated at 60° C. for 3 h. After completion (monitored by LCMS), the reaction mixture was quenched with cold water (10 mL) and extracted with DCM (2×10 mL). The combined organic layer was washed with brine (10 mL), dried over sodium sulphate, and concentrated to get the title compound (262-3, 0.209 g, 96% yield) as a light brown gum. ¹H NMR (400 MHz, DMSO-d₆) δ=8.46 (d, J=1.6 Hz, 2H), 7.66-7.57 (m, 4H), 4.98-4.91 (m, 1H), 4.51-4.42 (m, 1H), 4.33-4.23 (m, 1H), 4.08-3.98 (m, 3H), 2.44-2.23 (m, 13H), 2.38-2.22 (m, 1H), 1.71-1.62 (m, 2H), 1.18 (t, J=7.1 Hz, 3H). LCMS: (Method A) 582.0 (M+H)⁺, Rt. 1.74 min, 96.36% (Max).

### Step 2: ((R)-4-(4-(3-(3,6-dibromo-9H-carbazol-9-yl)-2-hydroxypropyl)piperazin-1-yl)butanoic acid dihydrochloride (262-4)

To a solution of ethyl (R)-4-(4-(3-(3,6-dibromo-9H-carbazol-9-yl)-2-hydroxypropyl)piperazin-1-yl)butanoate (262-3, 0.346 g, 0.59 mmol) in 1,4-dioxane (2.3 mL) at RT, a solution of NaOH (71 mg, 1.79 mmol) in water (1.15 mL) was added and the resulting reaction mixture was stirred at RT overnight. After completion (monitored by LCMS), the reaction mixture was concentrated under vacuum. The residue was dissolved in dioxane, a solution of 4 M HCl in dioxane (1.0 mL) was added and the mixture was stirred for 15 min at RT. The mixture was then concentrated to get the title compound (262-4, 0.373 g, 99% yield) as an off-white solid. 1H NMR (300 MHz, DMSO-d₆) δ=8.49-8.43 (m, 2H), 7.70-7.54 (m, 4H), 4.51-4.40 (m, 1H), 4.37-4.23 (m, 1H), 4.10-3.98 (m, 1H), 3.42-3.17 (m, 3H), 3.53-3.15 (m, 4H), 2.48-2.21 (m, 10H), 1.76-1.60 (m, 2H). LCMS: (Method A) 554.0 (M+H)⁺, Rt. 1.63 min, 99.0% (Max).

### Step 3: 4-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)butanamide (Compound 183)

To a stirred solution of (R)-4-(4-(3-(3,6-dibromo-9H-carbazol-9-yl)-2-hydroxypropyl)piperazin-1-yl)butanoic acid dihydrochloride (262-4, 0.248 g, 0.40 mmol) in DMF (2.5 mL), EDC·HCl (0.114 g, 0.59 mmol), 1-hydroxy-7-azabenzotriazole (81 mg, 0.59 mmol), N-methyl morpholine (0.218 mL, 1.98 mmol) were added at 0° C. and the resulting reaction mixture was stirred for 15 min at 0° C. Then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (262-5, 0.204 g, 0.40 mmol) was added at 0° C. and the reaction mixture stirred for 18 h at RT. After completion (monitored by LCMS), the reaction mixture was quenched with ice-water and extracted with DCM (2×10 mL). The combined organic layer was washed with brine and concentrated. The crude residue was purified by reverse phase preparative HPLC (Purification method: Xbridge C8 (19×250) mm, 5 micron; Mobile phase A: 10 mM Ammonium bicarbonate and Mobile phase B: Acetonitrile, Flow rate=14 mL/minute) to get the title compound (54.98 mg 13% yield), as a white solid. ¹H NMR (400 MHz, DMSO-d₆) δ=8.48-8.39 (m, 3H), 7.96 (d, J=10.0 Hz, 1H), 7.70-7.56 (m, 6H), 7.45 (d, J=8.5 Hz, 2H), 7.32-7.22 (m, 4H), 7.20-7.13 (m, 1H), 4.95 (d, J=5.0 Hz, 1H), 4.87 (d, J=5.0 Hz, 1H), 4.45 (dd, J=3.3, 14.8 Hz, 1H), 4.36 (d, J=6.0 Hz, 2H), 4.28 (dd, J=6.8, 14.8 Hz, 1H), 4.10 (s, 3H), 4.07-3.83 (m, 4H), 3.72-3.60 (m, 1H), 3.28-3.12 (m, 3H), 2.95-2.82 (m, 1H), 2.58 (d, J=7.0 Hz, 3H), 2.45-2.35 (m, 6H), 2.32-2.23 (m, 4H), 2.19 (t, J=7.5 Hz, 2H), 1.76-1.65 (m, 2H), 1.56-1.24 (m, 4H), 1.21 (d, J=7.0 Hz, 3H). LCMS: (Method C) 1049.9, (M+H)⁺, Rt. 2.84 min, 97.88% (Max). HPLC: (Method A) Rt. 4.34 min, 98.70%.

### Example 263. Synthesis of 3-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)propanamide (Compound 184)

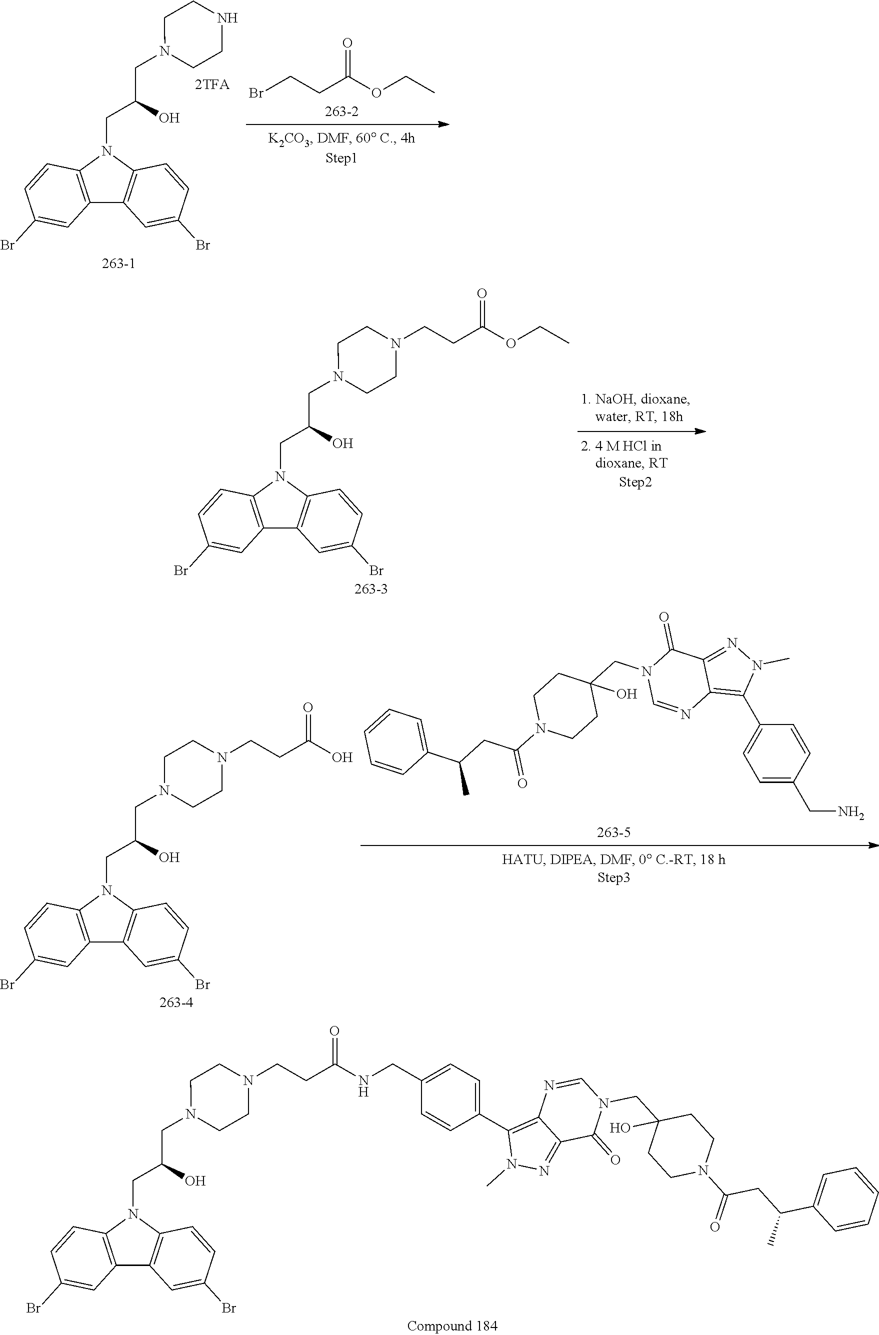

### Step 1: ethyl (R)-3-(4-(3-(3,6-dibromo-9H-carbazol-9-yl)-2-hydroxypropyl)piperazin-1-yl)propanoate (263-3)

To a solution of (R)-1-(3,6-dibromo-9H-carbazol-9-yl)-3-(piperazin-1-yl)propan-2-ol bis(2,2,2-trifluoroacetate) (263-1, 0.250 g, 0.36 mmol) in DMF (2.5 mL), potassium carbonate (75 mg, 0.54 mmol) and ethyl 3-bromopropanoate (263-2, 98 mg, 0.54 mmol) were added and the reaction mixture heated at 60° C. for 4 h. After completion (monitored by LCMS), the reaction mixture was quenched with cold water (10 mL) and extracted with DCM (2×10 mL). The combined organic layer was washed with brine (10 mL), dried over sodium sulphate, and concentrated to get the title compound (263-3, 0.173 g, 63% yield) as a pale-yellow gum. ¹H NMR (400 MHz, DMSO-d₆) δ=8.46 (d, J=2.0 Hz, 2H), 7.65-7.57 (m, 4H), 4.95 (d, J=5.5 Hz, 1H), 4.48-4.40 (m, 1H), 4.28 (dd, J=6.5, 15.0 Hz, 1H), 4.10-3.97 (m, 3H), 2.60-2.53 (m, 2H), 2.49-2.21 (m, 12H), 1.18 (t, J=7.0 Hz, 3H). LCMS: (Method A) 567.8 (M+H)⁺, Rt. 3.05 min, 74.95% (Max).

### Step 2: (R)-3-(4-(3-(3,6-dibromo-9H-carbazol-9-yl)-2-hydroxypropyl)piperazin-1-yl)propanoic acid dihydrochloride (263-4)

To a stirred solution of ethyl (R)-3-(4-(3-(3,6-dibromo-9H-carbazol-9-yl)-2-hydroxypropyl)piperazin-1-yl)propanoate (263-3, 0.17 g, 0.300 mmol) in 1,4-dioxane (1.0 mL), a solution of NaOH (71 mg, 1.79 mmol) in water (1.0 mL) was added and the resulting reaction mixture was stirred at RT overnight. After completion (monitored by LCMS), the reaction mixture was concentrated under vacuum. The residue was dissolved in 1,4-dioxane and a solution of 4 M HCl in dioxane was added, and the mixture was stirred at RT for 15 min. The mixture was concentrated to get the title compound (263-4, 0.170 g, 89% yield) as an off-white solid. 1H NMR (300 MHz, DMSO-d₆) δ=13.08-11.26 (m, 2H), 8.47 (d, J=1.8 Hz, 2H), 7.77 (d, J=8.8 Hz, 2H), 7.60 (dd, J=1.9, 8.8 Hz, 2H), 4.57-4.40 (m, 3H), 3.98-3.60 (m, 7H), 3.55-3.40 (m, 5H), 2.98-2.78 (m, 2H). LCMS: (Method A) 540.0 (M+H)⁺, Rt. 1.89 min, 96.21% (Max).

### Step 3: 4-(4-((R)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)butanamide Compound 184

To a solution of (R)-3-(4-(3-(3,6-dibromo-9H-carbazol-9-yl)-2-hydroxypropyl)piperazin-1-yl)propanoic acid dihydrochloride (263-4, 0.17 g, 0.278 mmol) in DMF (2.5 mL), DIPEA (0.243 mL, 1.39 mmol) and HATU (0.158 g, 0.42 mmol) were added at 0° C. and the resulting reaction mixture was stirred for 15 min at 0° C. Then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (0.143 g, 0.28 mmol) was added at 0° C. and the reaction mixture stirred at RT for 18 h. After completion (monitored by LCMS), the reaction was quenched with ice-water (10 mL) and extracted with DCM (2×10 mL). The combined organic layer was washed with brine and concentrated. The crude residue was purified by reverse phase preparative HPLC (Purification method: Xbridge C8 (19×250) mm, 5 micron; Mobile phase A: 10 mM Ammonium bicarbonate and Mobile phase B: Acetonitrile, Flow rate=12 mL/minute) to get the title compound (16.0 mg, 5.3% yield) as a pale-yellow solid. 1H NMR (400 MHz, DMSO-d₆) δ=8.55-8.49 (m, 1H), 8.45 (d, J=2.0 Hz, 2H), 7.96 (d, J=10.5 Hz, 1H), 7.68-7.58 (m, 6H), 7.49 (d, J=8.5 Hz, 2H), 7.31-7.22 (m, 4H), 7.20-7.13 (m, 1H), 4.98-4.84 (m, 2H), 4.48-4.42 (m, 1H), 4.40-4.34 (m, 2H), 4.32-4.24 (m, 1H), 4.10 (s, 3H), 4.06-3.87 (m, 4H), 3.71-3.58 (m, 1H), 3.27-3.09 (m, 2H), 2.94-2.81 (m, 1H), 2.63-2.56 (m, 4H), 2.44-2.25 (m, 12H), 1.57-1.23 (m, 4H), 1.23-1.18 (m, 3H). LCMS: (Method A) 1036.1, (M+H)⁺, Rt. 2.27 min, 97.96% (Max), HPLC: (Method A) Rt. 4.23 min, 96.18% (Max).

### Example 264. Synthesis of (R)—N1-(2-((3-((2-fluoro-9H-fluoren-9-ylidene)methyl)pyridin-2-yl)oxy)ethyl)-N4-(4-(6-((4-hydroxy-1-(3-phenylbutanoyl)piperidin-4-yl)methyl)-2-methyl-7-oxo-6,7-dihydro-2H-pyrazolo[4,3-d] pyrimidin-3-yl)benzyl)succinamide (Compound 185)

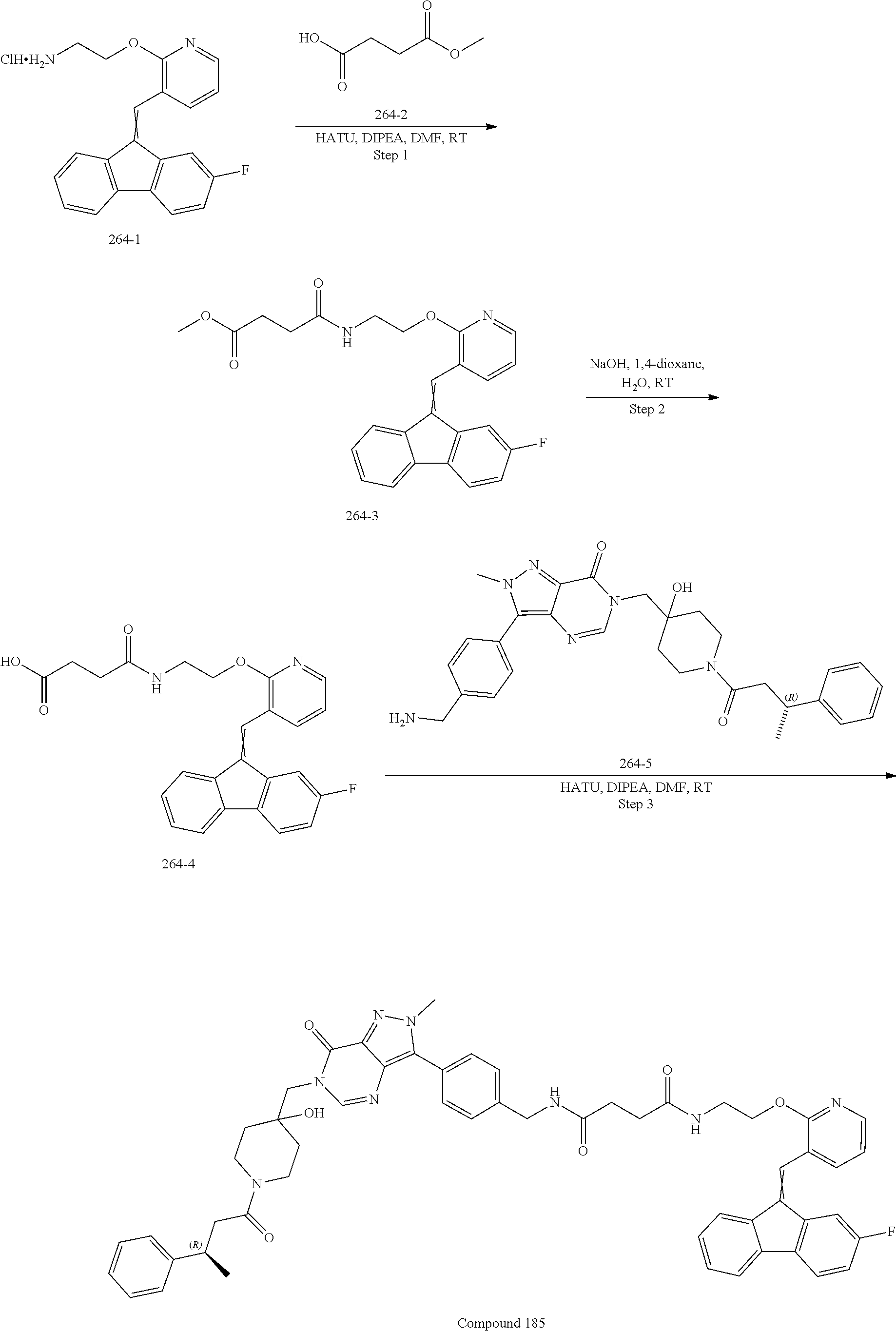

### Step 2: methyl 4-((2-((3-((2-fluoro-9H-fluoren-9-ylidene)methyl)pyridin-2-yl)oxy)ethyl)amino)-4-oxobutanoate (264-3)

To a stirred solution of 4-methoxy-4-oxobutanoic acid (264-2, 64 mg, 0.49 mmol) in DMF (3 mL) at RT were added DIPEA (0.18 mL, 1.02 mmol) and HATU (0.232 g, 0.61 mmol) and the reaction mixture was stirred for 10 minutes at RT. Then 2-((3-((2-fluoro-9H-fluoren-9-ylidene)methyl)pyridin-2-yl)oxy)ethan-1-amine hydrochloride (264-1, 0.15 g, 0.41 mmol) was added at 0° C. and the reaction mixture was stirred at RT overnight. The reaction was monitored by UPLC, which showed product formation. The reaction mixture was diluted with water (10 mL) and extracted with EtOAc (2×20 mL). The combined organic extract was washed with brine (5 mL), dried over anhydrous sodium sulphate, filtered, and concentrated under reduced pressure. The crude residue was purified by flash column chromatography (silica-gel, 100-200 mesh size) using hexane-EtOAc (50 to 80%) as an eluent to get the title compound (264-3, 0.12 g, 56% yield) as a yellow solid. ¹H NMR (400 MHz, CHLOROFORM-d) δ=8.30-8.21 (m, 1H), 8.06-7.95 (m, 1H), 7.72-7.65 (m, 2H), 7.59-7.52 (m, 1H), 7.50-7.47 (m, 1H), 7.51-7.32 (m, 3H), 7.18-7.04 (m, 3H), 6.25-6.07 (m, 1H), 4.60-4.47 (m, 2H), 3.75-3.72 (m, 1H), 3.70-3.61 (m, 5H), 2.84 (s, 4H), 2.64-2.58 (m, 2H), 2.40-2.30 (m, 2H). LCMS: (Method C) 447.0 (M+H)⁺, Rt. 2.82 min, 85.67% (Max).

### Step 3: 4-((2-((3-((2-fluoro-9H-fluoren-9-ylidene)methyl)pyridin-2-yl)oxy)ethyl)amino)-4-oxobutanoic acid (264-4)

To a stirred solution of methyl 4-((2-((3-((2-fluoro-9H-fluoren-9-ylidene)methyl)pyridin-2-yl)oxy)ethyl)amino)-4-oxobutanoate (264-3, 0.115 g, 0.26 mmol) in 1,4-dioxane (3 mL) and water (0.6 mL) at RT was added NaOH (21 mg, 0.51 mmol) and the reaction mixture was stirred at RT overnight. The reaction mixture was monitored by UPLC. After completion, the reaction mixture was concentrated under reduced pressure. The residue was cooled and acidified with 4 M HCl in 1,4-dioxane. The mixture was concentrated under vacuum to get the title compound (264-4, 90 mg, 64% yield) as a yellow solid. LCMS: (Method C) 432.9 (M+H), Rt. 2.15 min, 79.71% (Max).

### Step 4: (R)—N1-(2-((3-((2-fluoro-9H-fluoren-9-ylidene)methyl)pyridin-2-yl)oxy)ethyl)-N4-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)succinamide (Compound 185)

To a stirred solution of 4-((2-((3-((2-fluoro-9H-fluoren-9-ylidene)methyl)pyridin-2-yl)oxy)ethyl)amino)-4-oxobutanoic acid (264-4, 90 mg, 0.21 mmol) in DMF (3 mL) were added DIPEA (0.09 mL, 0.52 mmol) and HATU (119 mg, 0.31 mmol) at RT. After stirring for 10 min, (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (264-5, 129 mg, 0.25 mmol) was added at 0° C. and the reaction mixture was stirred at RT overnight. After completion (monitored by UPLC), the reaction mixture was diluted with water (10 mL) and extracted with EtOAc (2×20 mL). The combined organic extract was washed with brine (5 mL), dried over anhydrous sodium sulphate, filtered, and concentrated under reduced pressure. The crude residue was purified by reverse phase preparative HPLC purification (Purification method: X SELECT C18 (19×150) mm, 5 μm); Mobile phase A: 10 mM Ammonium bicarbonate/MeCN and Mobile phase B: MeCN in THF, Flow rate=14 mL/minute) to get the title compound (65 mg, 33% yield) as an off-white solid. ¹H NMR (400 MHz, DMSO-d₆) δ=8.49-8.38 (m, 1H), 8.29-8.21 (m, 1H), 8.09-7.82 (m, 6H), 7.79-7.72 (m, 1H), 7.70-7.57 (m, 2H), 7.46-7.38 (m, 3H), 7.38-7.32 (m, 1H), 7.30-7.22 (m, 5H), 7.19-7.09 (m, 3H), 4.88 (d, J=5.0 Hz, 1H), 4.42-4.29 (m, 4H), 4.12-3.85 (m, 6H), 3.71-3.59 (m, 1H), 3.45-3.41 (m, 2H), 3.27-3.10 (m, 2H), 2.93-2.81 (m, 1H), 2.65-2.56 (m, 2H), 2.44-2.33 (m, 4H), 1.59-1.24 (m, 4H), 1.21 (d, J=7.0 Hz, 3H). LCMS: (Method C) 929.0 (M+H)⁺, Rt. 2.72 min, 97.29% (Max). HPLC: (Method A) Rt. 4.96 min, 98.40% (Max).

### Example 265. Synthesis of (R)—N1-(2-((3-((2-fluoro-9H-fluoren-9-ylidene)methyl)pyridin-2-yl)oxy)ethyl)-N6-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)adipamide (Compound 186)

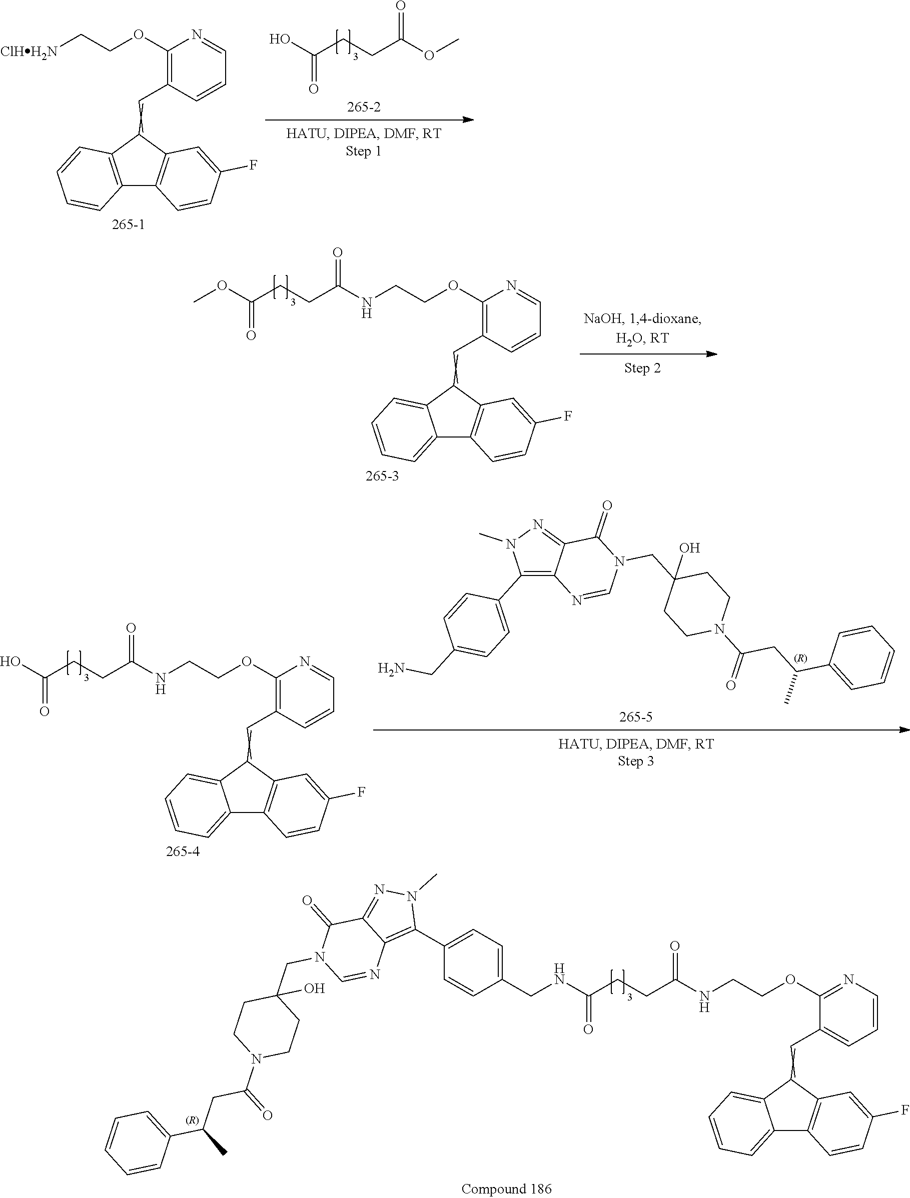

### Step 1: Methyl 6-((2-((3-((2-fluoro-9H-fluoren-9-ylidene)methyl)pyridin-2-yl)oxy)ethyl)amino)-6-oxohexanoate (265-3)

To a stirred solution of 6-methoxy-6-oxohexanoic acid (265-2, 78 mg, 0.49 mmol) in DMF (3 mL) at RT were added DIPEA (0.131 g, 1.02 mmol) and HATU (0.232 g, 0.61 mmol) and the reaction mixture was stirred for at RT 10 min. Then 2-((3-((2-fluoro-9H-fluoren-9-ylidene)methyl)pyridin-2-yl)oxy)ethan-1-amine hydrochloride (265-1, 0.15 g, 0.41 mmol) was added at 0° C. and the reaction mixture was stirred at RT overnight. The reaction mixture was monitored by UPLC which showed the desired product formation. The reaction mixture was diluted with water (10 mL) and extracted with EtOAc (2×20 mL). The combined organic extract was washed with brine (5 mL), dried over anhydrous sodium sulphate, filtered, and concentrated under reduced pressure. The crude residue was purified by flash column chromatography (silica-gel, 100-200 mesh size) using hexane-EtOAc (50 to 80%) as an eluent to obtain the title compound (265-3, 0.13 g, 62% yield) as a yellow solid. ¹H NMR (400 MHz, CHLOROFORM-d) δ=8.30-8.20 (m, 1H), 7.98-7.89 (m, 1H), 7.73-7.64 (m, 2H), 7.59-7.45 (m, 2H), 7.44-7.34 (m, 2H), 7.17-7.02 (m, 3H), 6.03-5.75 (m, 1H), 4.58-4.47 (m, 2H), 3.68-3.60 (m, 5H), 2.29-2.20 (m, 2H), 1.56-1.53 (m, 2H). LCMS: (Method C) 475.0 (M+H)⁺, Rt. 2.87 min, 91.82% (Max).

### Step 2: 6-((2-((3-((2-fluoro-9H-fluoren-9-ylidene)methyl)pyridin-2-yl)oxy)ethyl)amino)-6-oxohexanoic acid (265-4)

To a stirred solution of methyl 6-((2-((3-((2-fluoro-9H-fluoren-9-ylidene)methyl)pyridin-2-yl)oxy)ethyl)amino)-6-oxohexanoate (265-3, 0.115 g, 0.24 mmol) in 1,4-dioxane (3 mL) and water (0.6 mL) at RT was added NaOH (0.019 g, 0.48 mmol) and the reaction mixture was stirred at RT overnight. After completion (monitored by UPLC), the reaction mixture was concentrated. The residue was cooled to 0° C., diluted with cold water (1 mL), and acidified with 4 N HCl in 1,4-dioxane. The mixture was concentrated under vacuum to get the title compound (265-4, 95 mg, 52% yield) as a yellow solid. 1H NMR (400 MHz, DMSO-d₆) δ=8.31-8.23 (m, 1H), 8.11-7.82 (m, 5H), 7.79-7.73 (m, 1H), 7.48-7.32 (m, 2H), 7.29-7.21 (m, 1H), 7.19-7.04 (m, 2H), 4.39-4.33 (m, 2H), 3.47-3.37 (m, 2H), 2.41-2.35 (m, 2H), 2.32-2.23 (m, 2H). LCMS: (Method C) 461.0 (M+H)⁺, Rt. 2.17 min, 61.62% (Max).

### Step 3: (R)—N1-(2-((3-((2-fluoro-9H-fluoren-9-ylidene)methyl)pyridin-2-yl)oxy)ethyl)-N6-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl) adipamide (Compound 186)

To a stirred solution of 6-((2-((3-((2-fluoro-9H-fluoren-9-ylidene)methyl)pyridin-2-yl)oxy)ethyl)amino)-6-oxohexanoic acid (265-4, 90 mg, 0.19 mmol) in DMF (3 mL) at RT were added DIPEA (0.084 mL, 63 mg, 0.49 mmol), HATU (0.111 g, 0.29 mmol), and the reaction mixture was stirred for 10 min at RT. Then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 0.121 g, 0.23 mmol) was added at 0° C. and the reaction mixture was stirred at RT overnight. The reaction mixture was diluted with water (10 mL) and extracted with EtOAc (2×20 mL). The combined organic extract was washed with brine (5 mL), dried over anhydrous sodium sulphate, filtered, and concentrated under reduced pressure. The resulting crude residue was purified by reverse phase preparative HPLC purification (Purification method: X SELECT C18 (19×250) mm 5 micron; Mobile phase A: 10 mM Ammonium bicarbonate/MeCN and Mobile phase B: MeCN in THF, Flow rate=12 mL/minute) to get the title compound (42 mg, 22% yield) as an off-white solid. 1H NMR (400 MHz, DMSO-d₆) δ=8.41-8.33 (m, 1H), 8.30-8.21 (m, 1H), 8.05-7.83 (m, 6H), 7.77-7.71 (m, 1H), 7.67-7.62 (m, 2H), 7.47-7.34 (m, 4H), 7.30-7.21 (m, 5H), 7.18-7.07 (m, 3H), 4.91-4.84 (m, 1H), 4.43-4.30 (m, 4H), 4.12-4.07 (m, 3H), 4.07-3.84 (m, 3H), 3.72-3.59 (m, 1H), 3.47-3.37 (m, 2H), 3.27-3.13 (m, 2H), 2.94-2.80 (m, 1H), 2.65-2.56 (m, 2H), 2.20-1.99 (m, 4H), 1.52-1.27 (m, 8H), 1.24-1.18 (m, 3H). LCMS: (Method C) 957.0 (M+H)⁺, Rt. 2.72 min, 95.59% (Max). HPLC: (Method A) Rt. 4.99 min, 98.31% (Max).

### Example 266. Synthesis of (R)—N1-(2-((3-((2-fluoro-9H-fluoren-9-ylidene)methyl)pyridin-2-yl)oxy)ethyl)-N8-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)octanediamide (Compound 187)

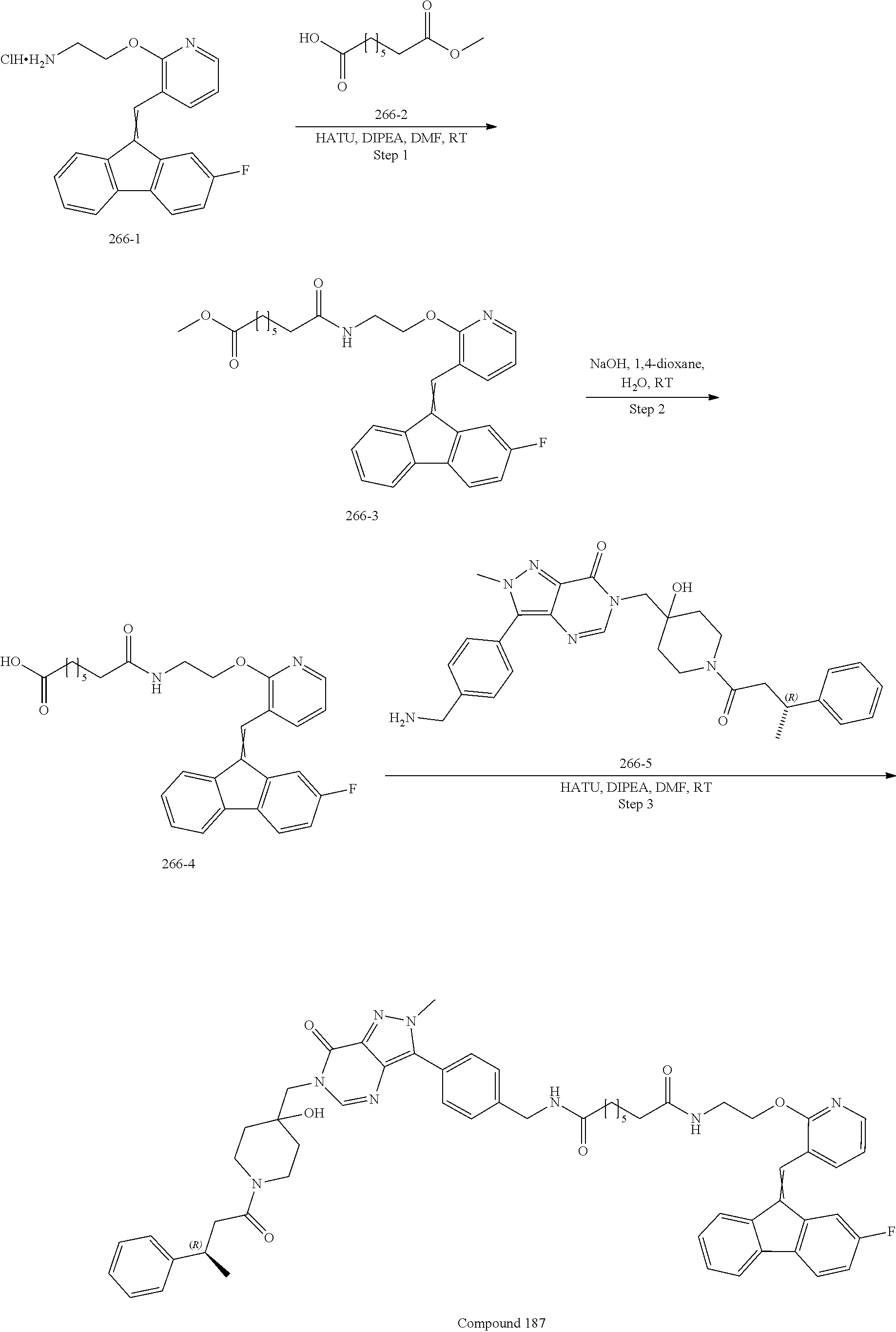

### Step 1: Methyl 8-((2-((3-((2-fluoro-9H-fluoren-9-ylidene)methyl)pyridin-2-yl)oxy)ethyl)amino)-8-oxooctanoate (266-3)

To a stirred solution of 8-methoxy-8-oxooctanoic acid (266-2, 73 mg, 0.39 mmol) in DMF (3 mL) at RT were added DIPEA (0.142 mL, 0.81 mmol), HATU (0.186 g, 0.49 mmol), and the reaction mixture was stirred for 10 min at RT. Then 2-((3-((2-fluoro-9H-fluoren-9-ylidene)methyl)pyridin-2-yl)oxy)ethan-1-amine hydrochloride (266-1, 0.12 g, 0.32 mmol) was added at 0° C. and the reaction mixture was stirred at RT overnight. The reaction mixture was diluted with water (5 mL) and extracted with EtOAc (2×15 mL). The combined organic extract was washed with brine (2 mL), dried over anhydrous sodium sulphate, filtered, and concentrated under reduced pressure. The crude residue was purified by flash column chromatography (silica-gel, 240-400 mesh size) using hexane-EtOAc (60 to 70%) as an eluent to obtain the title compound (266-3, 0.12 g, 71% yield) as a yellow solid. ¹H NMR (300 MHz, DMSO-d₆) δ=8.33-8.21 (m, 1H), 8.09-8.01 (m, 1H), 8.00-7.78 (m, 4H), 7.78-7.68 (m, 1H), 7.53-7.31 (m, 2H), 7.31-7.20 (m, 1H), 7.20-7.06 (m, 2H), 4.45-4.27 (m, 2H), 3.55 (d, J=1.6 Hz, 3H), 3.42 (q, J=5.5 Hz, 2H), 2.24-2.10 (m, 2H), 2.06-1.93 (m, 2H), 1.54-1.32 (m, 4H), 1.28-1.06 (m, 4H). LCMS: (Method C) 503.0 (M+H), Rt. 2.98 min, 96.98% (Max).

### Step 2: 8-((2-((3-((2-fluoro-9H-fluoren-9-ylidene)methyl)pyridin-2-yl)oxy)ethyl)amino)-8-oxooctanoic acid (266-4)

To a stirred solution of methyl 8-((2-((3-((2-fluoro-9H-fluoren-9-ylidene)methyl)pyridin-2-yl)oxy)ethyl)amino)-8-oxooctanoate (266-3, 0.115 g, 0.23 mmol) in 1,4-dioxane-water (5 mL:1 mL) at 0° C. was added NaOH (18 mg, 0.46 mmol) and the reaction mixture was stirred at RT overnight. The reaction mixture was monitored by UPLC. After completion, the reaction mixture was concentrated, and the residue was acidified with 4 M HCl in 1,4-dioxane. The resulting mixture was concentrated and dried under vacuum to get the title compound (266-4, 0.12 g, 95% yield) as a yellow solid. LCMS: (Method C) 489.2 (M+H)⁺, Rt. 2.02 min, 88.92% (Max).

### Step 3: (R)—N1-(2-((3-((2-fluoro-9H-fluoren-9-ylidene)methyl)pyridin-2-yl)oxy)ethyl)-N8-(4-(6-((4-hydroxy-1-(3-phenylbutanoyl)piperidin-4-yl)methyl)-2-methyl-7-oxo-6,7-dihydro-2H-pyrazolo[4,3-d]pyrimidin-3-yl)benzyl)octanediamide (Compound 187)

To a stirred solution of 8-((2-((3-((2-fluoro-9H-fluoren-9-ylidene)methyl)pyridin-2-yl)oxy)ethyl)amino)-8-oxooctanoic acid (266-4, 0.11 g, 0.22 mmol) in DMF (3 mL) at RT were added DIPEA (0.1 mL, 0.56 mmol), HATU (0.128 g, 0.34 mmol) and the reaction mixture was stirred for 10 min at 0° C. Then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 0.139 g, 0.27 mmol) was added at 0° C. and the reaction mixture was stirred at RT overnight. After completion (monitored by TLC), the reaction mixture was diluted with water (10 mL) and extracted with EtOAc (2×20 mL). The combined organic extract was washed with brine (5 mL), dried over anhydrous sodium sulphate, filtered, and concentrated under reduced. The crude residue was purified by reverse phase preparative HPLC purification (Purification method: X BRIDGE C8 (19×150) mm, 5 μm); Mobile phase A: 10 mM Ammonium bicarbonate/MeCN and Mobile phase B: MeCN in THF, Flow rate=14 mL/minute). The fractions were concentrated, the residue was diluted with DCM, and washed with sat NaHCO₃ solution. The organic layer was concentrated and lyophilized to get the title compound (35.5 mg, 16% yield) as a pale-yellow solid. ¹H NMR (400 MHz, DMSO-d₆) δ=8.43-8.32 (m, 1H), 8.23 (s, 1H), 8.07-8.01 (m, 1H), 7.99-7.82 (m, 5H), 7.76-7.71 (m, 1H), 7.68-7.61 (m, 2H), 7.50-7.33 (m, 4H), 7.31-7.20 (m, 5H), 7.19-7.05 (m, 3H), 4.87 (d, J=5.0 Hz, 1H), 4.42-4.29 (m, 4H), 4.13-3.86 (m, 6H), 3.73-3.60 (m, 1H), 3.45-3.38 (m, 2H), 3.24-3.12 (m, 2H), 2.93-2.82 (m, 1H), 2.65-2.55 (m, 2H), 2.14-2.05 (m, 2H), 2.04-1.95 (m, 2H), 1.51-1.28 (m, 7H), 1.21 (d, J=6.5 Hz, 3H), 1.21-1.06 (m, 4H). LCMS: (Method B) 985.5 (M+H), Rt. 2.18 min, 99.94% (Max). HPLC: (Method A) Rt. 5.08 min, 99.94% (Max).

### Example 267. Synthesis of 4-(4-((S)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-4-oxobutanamide (Compound 188)

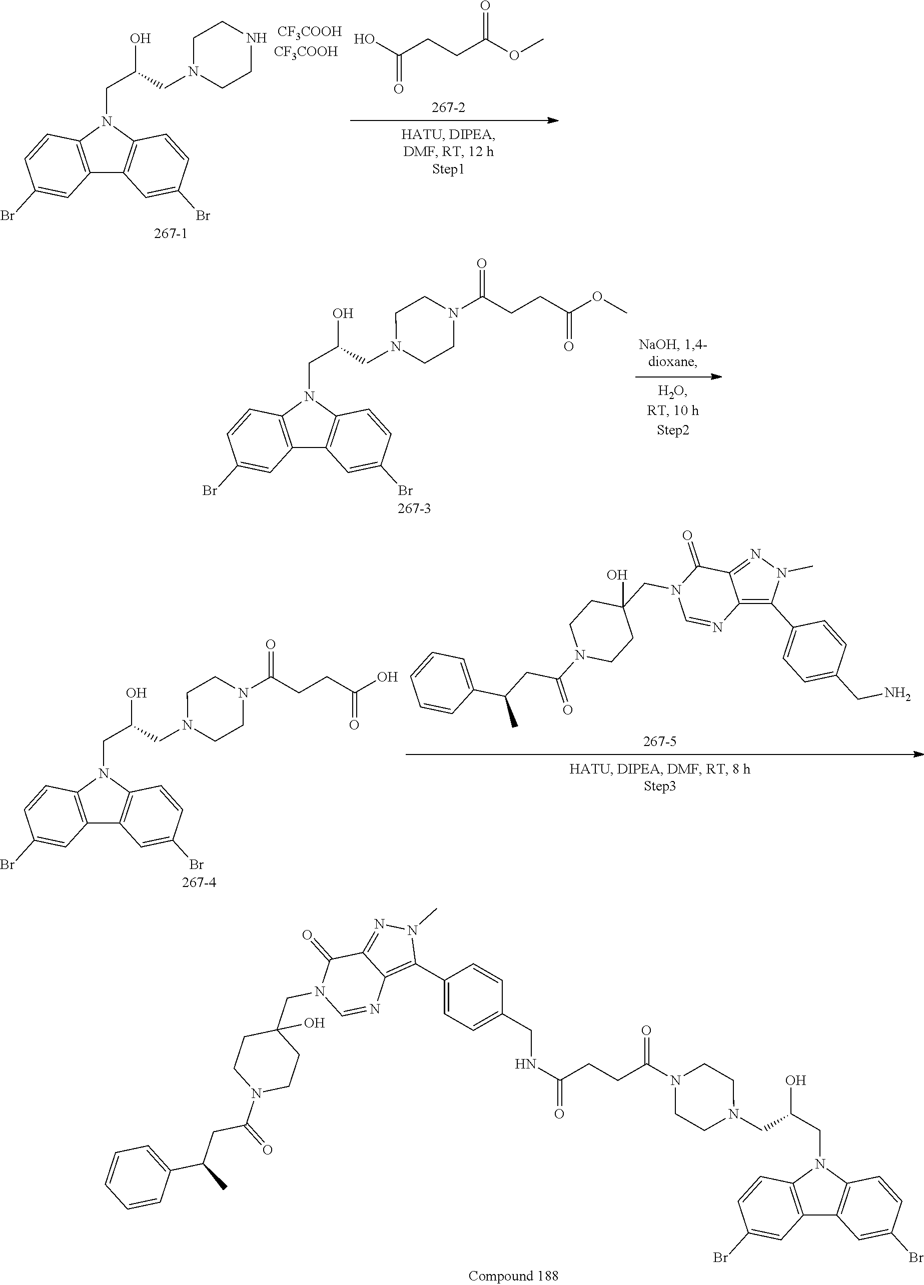

### Step 1: Methyl (R)-4-(4-(3-(3,6-dibromo-9H-carbazol-9-yl)-2-hydroxypropyl)piperazin-1-yl)-4-oxobutanoate (267-3)

To a stirred solution of 4-methoxy-4-oxobutanoic acid (267-2, 25 mg, 0.19 mmol) in DMF (0.2 mL) at 0° C. were added DIPEA (0.033 mL, 0.19 mmol) and HATU (72 mg, 0.19 mmol) followed by the addition of (R)-1-(3,6-dibromo-9H-carbazol-9-yl)-3-(piperazin-1-yl)propan-2-ol bis(2,2,2-trifluoroacetate) (267-1, 0.132 g, 0.19 mmol). The reaction mixture was stirred at RT for 12 h. After completion (monitored by LCMS), the reaction mixture was diluted with ice-cold water (5 mL) and extracted with DCM (2×5 mL). The combined organic extract was washed with water (5 mL), dried over anhydrous sodium sulphate, filtered and concentrated under reduced pressure to afford methyl (R)-4-(4-(3-(3,6-dibromo-9H-carbazol-9-yl)-2-hydroxypropyl)piperazin-1-yl)-4-oxobutanoate (267-3, 80 mg, 51% yield) as a brown liquid. ¹H NMR (300 MHz, DMSO-d₆) δ=8.50-8.41 (m, 2H), 7.70-7.53 (m, 4H), 5.07-4.91 (m, 1H), 4.55-4.41 (m, 1H), 4.38-4.22 (m, 1H), 4.15-3.98 (m, 1H), 3.57 (s, 3H), 3.42 (br s, 4H), 2.60-2.54 (m, 2H), 2.44-2.30 (m, 6H). LCMS: (Method C) 581.8 (M+H)⁺, Rt. 2.89 min, 67.55% (Max).

### Step 2: (R)-4-(4-(3-(3,6-dibromo-9H-carbazol-9-yl)-2-hydroxypropyl)piperazin-1-yl)-4-oxobutanoic acid (267-4)

To a stirred solution of methyl (R)-4-(4-(3-(3,6-dibromo-9H-carbazol-9-yl)-2-hydroxypropyl)piperazin-1-yl)-4-oxobutanoate (267-3, 0.2 g, 0.34 mmol) in 1,4-dioxane (2 mL) was added a solution of NaOH (41 mg, 1.03 mmol) in water (1.0 mL) and the reaction mixture was stirred at RT for 10 h. After completion (monitored by LCMS), the reaction mixture was concentrated under reduced pressure and the residue was dried by azeotropic co-distillation with toluene (2×3 mL) to get the title compound (267-4, 0.17 g, 71% yield) as a pale-yellow solid. LCMS: (Method C) 567.8 (M+H)⁺, Rt. 2.26 min, 81.23% (Max).

### Step 3: 4-(4-((S)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-4-oxobutanamide (Compound 188)

To a stirred solution of (R)-4-(4-(3-(3,6-dibromo-9H-carbazol-9-yl)-2-hydroxypropyl)piperazin-1-yl)-4-oxobutanoic acid (267-4, 0.15 g, 0.26 mmol) in DMF (3 mL) at 0° C. were added DIPEA (0.171 g, 1.32 mmol) followed by HATU (0.151 g, 0.40 mmol). The reaction mixture was stirred for 10 min and then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 0.136 g, 0.26 mmol) was added and the reaction mixture was stirred at RT for 8 h. The reaction mixture was diluted with ice-cold water (5 mL), upon which solid precipitated. The solid was collected through filtration (110 mg) and then purified by reverse phase preparative HPLC purification (Purification method: X-bridge C8 (19×150) mm, 5 μm); Mobile phase A: 0.1% TFA in water and Mobile phase B: MeCN, Flow rate=12 mL/minute). The fractions were concentrated, and the residue was treated with aq. NaHCO₃ solution. The mixture was extracted with DCM (2×8 mL) and the combined organic layer was dried over anhydrous Na₂SO₄, filtered, concentrated, and then lyophilized to get the title compound (25 mg, 9% yield) as an off-white solid. 1H NMR (400 MHz, DMSO-d₆) δ=8.51-8.40 (m, 3H), 7.98 (d, J=10.0 Hz, 1H), 7.71-7.56 (m, 6H), 7.46 (d, J=8.5 Hz, 2H), 7.31-7.23 (m, 4H), 7.21-7.11 (m, 1H), 5.01 (d, J=5.0 Hz, 1H), 4.87 (d, J=5.0 Hz, 1H), 4.54-4.43 (m, 1H), 4.39-4.24 (m, 3H), 4.15-4.09 (m, 3H), 4.08-3.86 (m, 4H), 3.72-3.60 (m, 1H), 3.51-3.42 (m, 4H), 3.26-3.12 (m, 2H), 3.06-2.96 (m, 1H), 2.93-2.80 (m, 1H), 2.64-2.56 (m, 4H), 2.46-2.37 (m, 6H), 1.59-1.45 (m, 1H), 1.42-1.28 (m, 3H), 1.23-1.19 (m, 3H). LCMS: (Method B) 1064.2 (M+H)⁺, Rt. 1.95 min, 99.05% (Max). HPLC: (Method A) Rt. 4.47 min, 99.17% (Max).

### Example 268. Synthesis of 6-(4-((S)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-6-oxohexanamide (Compound 189)

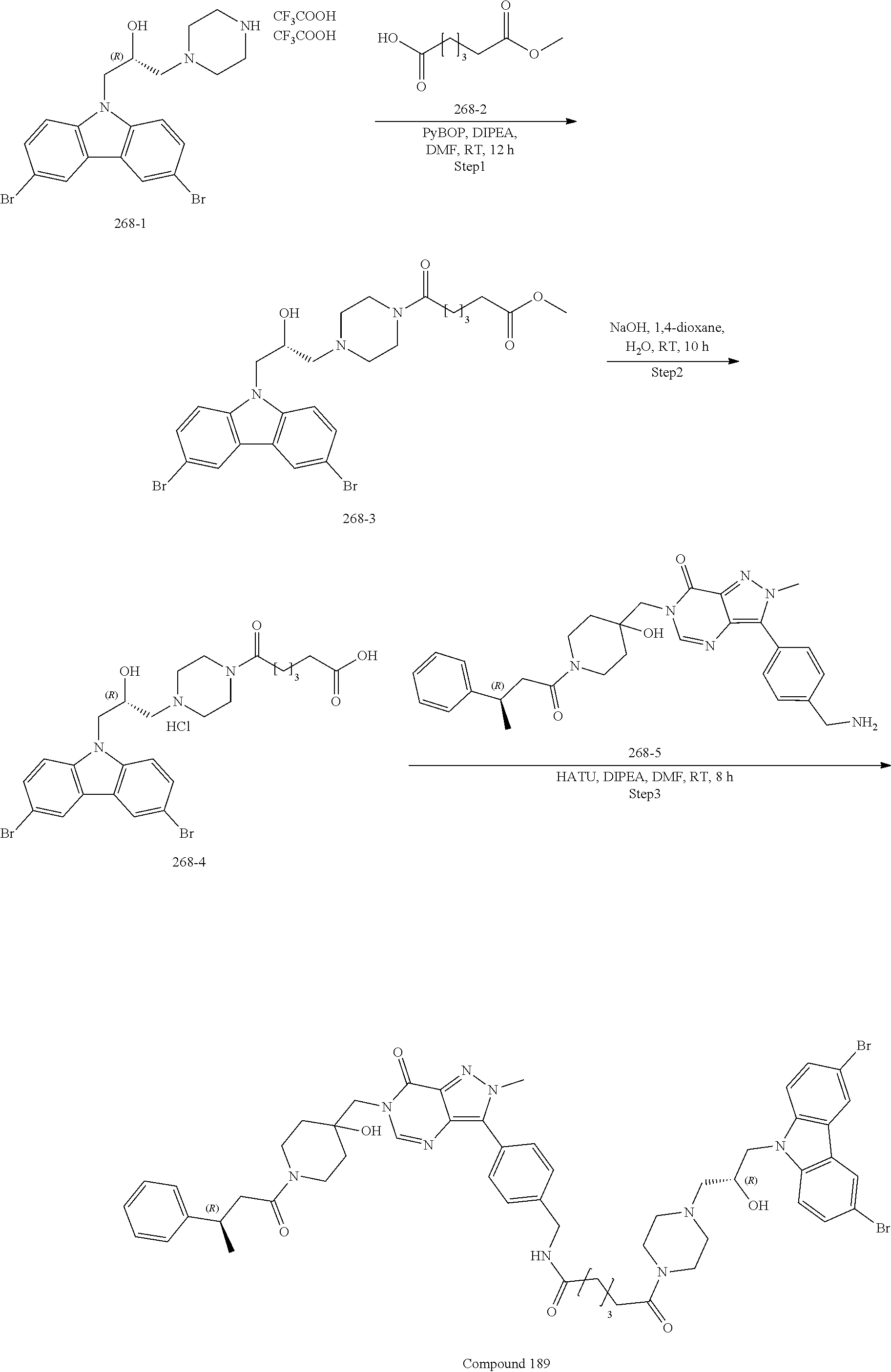

### Step 1: Methyl (R)-6-(4-(3-(3,6-dibromo-9H-carbazol-9-yl)-2-hydroxypropyl)piperazin-1-yl)-6-oxohexanoate (268-3)

To a stirred solution of 6-methoxy-6-oxohexanoic acid (268-2, 59 mg, 0.37 mmol) in DMF (3 mL) at 0° C. were added DIPEA (0.32 mL, 1.85 mmol) and PyBOP (0.193 g, 0.37 mmol) followed by the addition of (R)-1-(3,6-dibromo-9H-carbazol-9-yl)-3-(piperazin-1-yl)propan-2-ol bis(2,2,2-trifluoroacetate) (268-1, 0.2 g, 0.37 mmol). The reaction mixture was stirred at RT for 5 h. After completion (monitored by LCMS), the reaction mixture was diluted with water (10 mL) and extracted with DCM (2×10 mL). The combined organic extract was washed with water (10 mL), dried over anhydrous sodium sulphate, filtered, and concentrated under reduced pressure to get the title compound (268-3, 0.17 g, 49% yield) as a brown liquid. LCMS: (Method C) 609.8 (M+H)⁺, Rt. 2.94 min, 72.40% (Max).

### Step 2: (R)-6-(4-(3-(3,6-dibromo-9H-carbazol-9-yl)-2-hydroxypropyl)piperazin-1-yl)-6-oxohexanoic acid hydrochloride (268-4)

To a stirred solution of methyl (R)-6-(4-(3-(3,6-dibromo-9H-carbazol-9-yl)-2-hydroxypropyl)piperazin-1-yl)-6-oxohexanoate (268-3, 0.22 g, 0.36 mmol) in 1,4-dioxane (2 mL) was added a solution of NaOH (43 mg, 1.08 mmol) in water (1.0 mL) and the reaction mixture was stirred at RT for 10 h. As the reaction was not complete, more NaOH (29 mg, 0.72 mmol) was added, and the reaction mixture was heated to 50° C. for 4 h. After completion (monitored by LCMS), the reaction mixture was concentrated under reduced pressure. The residue was acidified with 4 M HCl in 1,4-dioxane and then dried under vacuum to get the title compound (268-4, 0.16 g, 70% yield) as a pale-yellow solid. ¹H NMR (400 MHz, DMSO-d₆) δ=8.51-8.36 (m, 2H), 7.75-7.45 (m, 4H), 5.22-4.89 (m, 1H), 4.55-4.40 (m, 1H), 4.39-4.26 (m, 1H), 4.12-3.98 (m, 1H), 3.12-2.95 (m, 2H), 2.44-2.10 (m, 10H), 1.60-1.37 (m, 4H). LCMS: (Method B) 596.1 (M+H)⁺, Rt. 1.84 min, 86.78% (Max).

### Step 3: 6-(4-((S)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-6-oxohexanamide (Compound 189)

To a stirred solution of (R)-6-(4-(3-(3,6-dibromo-9H-carbazol-9-yl)-2-hydroxypropyl)piperazin-1-yl)-6-oxohexanoic acid hydrochloride (268-4, 0.15 g, 0.24 mmol) in DMF (3 mL) at 0° C. were added DIPEA (0.041 mL, 0.24 mmol) followed by HATU (90 mg, 0.24 mmol). The reaction mixture was stirred for 10 min and then (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (5, 0.122 g, 0.24 mmol) was added and the reaction mixture was stirred at RT for 8 h. The reaction mixture was diluted with ice-cold water (10 mL), upon which solid precipitated. The solid was collected through filtration and then purified by reverse phase preparative HPLC purification [Purification method: Kromasil C18 (250×21.2) mm, 5 micron; Mobile phase A: 10 mM Ammonium bicarbonate in H₂O and Mobile phase B: MeCN, Flow rate-15 mL/min] to get the title compound (50 mg, 18% yield) as a white solid. ¹H NMR (400 MHz, DMSO-d₆) δ=8.51-8.38 (m, 3H), 7.97 (d, J=10.3 Hz, 1H), 7.70-7.56 (m, 6H), 7.44 (d, J=8.4 Hz, 2H), 7.33-7.22 (m, 4H), 7.19-7.12 (m, 1H), 5.00 (br d, J=4.6 Hz, 1H), 4.87 (d, J=5.1 Hz, 1H), 4.46 (br dd, J=3.8, 15.1 Hz, 1H), 4.39-4.27 (m, 3H), 4.10 (s, 3H), 4.08-3.87 (m, 4H), 3.73-3.60 (m, 1H), 3.49-3.39 (m, 4H), 3.26-3.13 (m, 2H), 2.94-2.84 (m, 1H), 2.65-2.56 (m, 2H), 2.41-2.27 (m, 8H), 2.24-2.14 (m, 2H), 1.63-1.44 (m, 5H), 1.41-1.25 (m, 3H), 1.23-1.18 (m, 3H). LCMS: (Method C) 1091.8 (M+H)⁺, Rt. 2.80 min, 95.04% (Max). HPLC: (Method A) Rt. 4.47 min, 93.69% (Max).

### Example 269. Synthesis of 8-(4-((S)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-8-oxooctanamide (Compound 190)

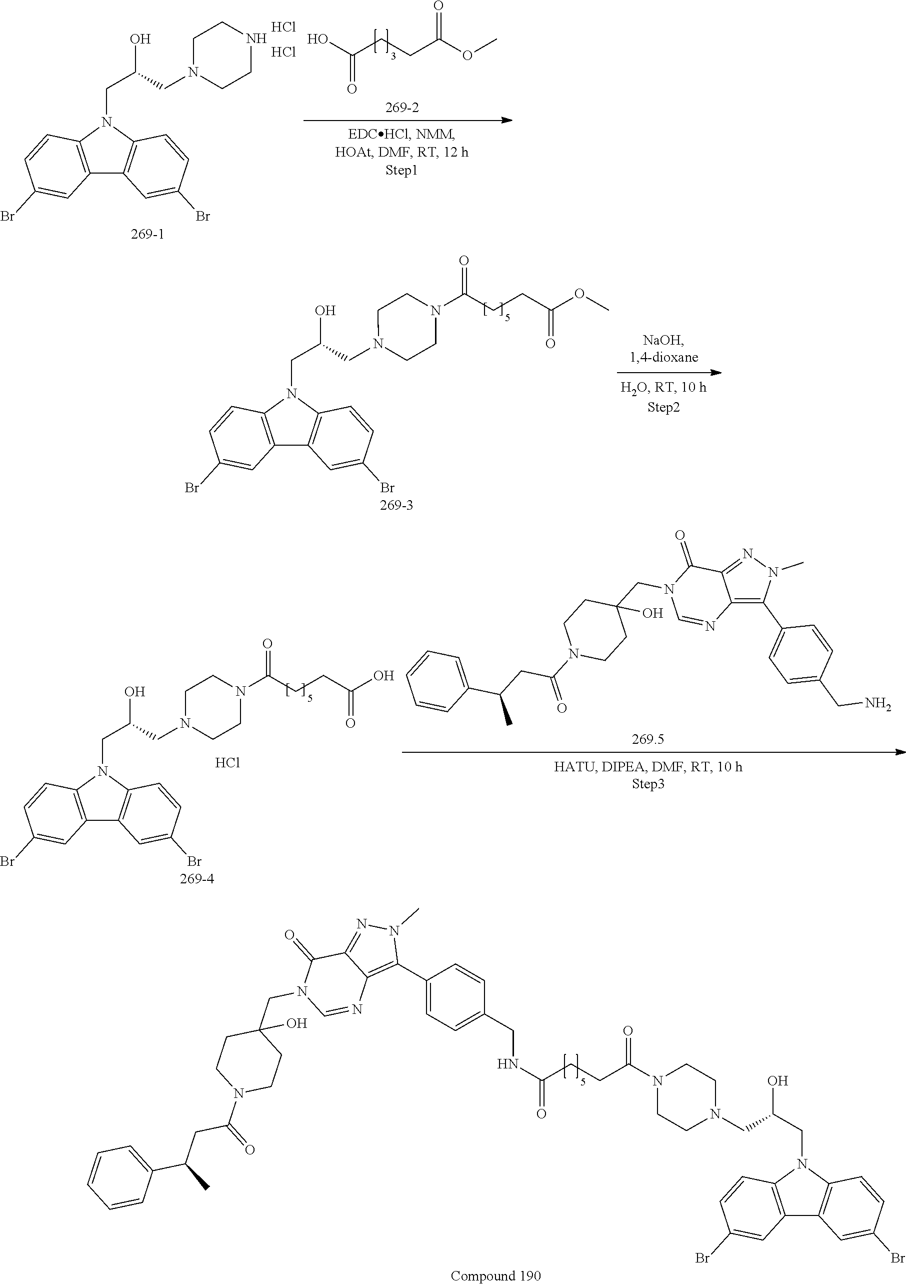

### Step 1: Methyl (R)-8-(4-(3-(3,6-dibromo-9H-carbazol-9-yl)-2-hydroxypropyl)piperazin-1-yl)-8-oxooctanoate (269-3)

To a stirred solution of 8-methoxy-8-oxooctanoic acid (269-2, 52 mg, 0.28 mmol) in DMF (2 mL) were added 1-hydroxy-7-azabenzotriazole (0.058 mL, 0.42 mmol) and N-methylmorpholine (0.15 mL, 1.39 mmol) and EDC·HCl (80 mg, 0.42 mmol) at 0° C. and the reaction mixture was stirred for 10 min at the same temperature. Then (R)-1-(3,6-dibromo-9H-carbazol-9-yl)-3-(piperazin-1-yl)propan-2-ol dihydrochloride (269-1, 0.15 g, 0.28 mmol) was added and the reaction mixture was stirred at RT for 8 h. After completion (monitored by LCMS), the reaction mixture was diluted with ice-cold water (8 mL) and extracted with DCM (2×8 mL). The combined organic extract was washed with water (8 mL), dried over anhydrous sodium sulphate, filtered, and concentrated under reduced pressure to get the title compound (269-3, 0.15 g, 64% yield) as a yellow solid. 1H NMR (300 MHz, DMSO-d₆) δ=8.46 (d, J=1.3 Hz, 2H), 7.68-7.57 (m, 4H), 5.00 (d, J=4.8 Hz, 1H), 4.59-4.22 (m, 2H), 4.17-3.88 (m, 1H), 3.58 (s, 3H), 3.48-3.39 (m, 4H), 2.39-2.26 (m, 10H), 1.58-1.43 (m, 4H), 1.30-1.21 (m, 4H). LCMS: (Method B) 638.0 (M+H)⁺, Rt. 2.30 min, 78.92% (Max).

### Step 2: (R)-8-(4-(3-(3,6-dibromo-9H-carbazol-9-yl)-2-hydroxypropyl)piperazin-1-yl)-8-oxooctanoic acid (269-4)

To a stirred solution of methyl (R)-8-(4-(3-(3,6-dibromo-9H-carbazol-9-yl)-2-hydroxypropyl)piperazin-1-yl)-8-oxooctanoate (269-3, 0.2 g, 0.31 mmol) in 1,4-dioxane (2 mL) was added NaOH (63 mg, 1.57 mmol) dissolved in water (1.0 mL) and the reaction mixture was stirred at RT for 10 h. After completion (monitored by LCMS), the reaction mixture was concentrated under vacuum and then acidified using 4 M HCl in 1,4-dioxane. The acidified mixture was concentrated under vacuum to get the title compound (269-4, 0.185 g, 89% yield) as an off-white solid. LCMS: (Method C) 623.8 (M+H)⁺, Rt. 2.34 min, 77.29% (Max).

### Step 3: 8-(4-((S)-3-(3,6-dibromo-9H-carbazol-9-yl)-2-hydroxypropyl)piperazin-1-yl)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)-8-oxooctanamide (Compound 190)

To a stirred solution of (R)-8-(4-(3-(3,6-dibromo-9H-carbazol-9-yl)-2-hydroxypropyl)piperazin-1-yl)-8-oxooctanoic acid (269-4, 0.18 g, 0.29 mmol) in DMF (3 mL) at 0° C., were added DIPEA (0.25 mL, 1.44 mmol) and HATU (0.165 g, 0.43 mmol). After 10 minutes of stirring at 0° C., (R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (269-5, 0.149 g, 0.29 mmol) was added and the reaction mixture was stirred at RT for 10 h. After completion (monitored by LCMS), the reaction mixture was diluted with ice-cold water (10 mL). The precipitated solid was collected by filtration. The crude product was purified by reverse phase preparative HPLC purification (Purification method: Kinetics (19×150) mm, 5 μm); Mobile phase A: 10 mM Ammonium bicarbonate/MeCN and Mobile phase B: MeCN in THF, Flow rate=15 mL/minute) to get the title compound (70 mg, 20% yield) as an off-white solid. ¹H NMR (400 MHz, DMSO-d₆) δ=8.50-8.44 (m, 2H), 8.41 (t, J=6.0 Hz, 1H), 7.97 (d, J=10.5 Hz, 1H), 7.71-7.57 (m, 6H), 7.44 (d, J=8.5 Hz, 2H), 7.31-7.22 (m, 4H), 7.20-7.12 (m, 1H), 5.00 (d, J=5.0 Hz, 1H), 4.87 (d, J=5.5 Hz, 1H), 4.52-4.24 (m, 4H), 4.10 (s, 3H), 4.08-3.85 (m, 4H), 3.72-3.59 (m, 1H), 3.46-3.38 (m, 4H), 3.26-3.13 (m, 3H), 2.93-2.82 (m, 1H), 2.66-2.54 (m, 2H), 2.40-2.24 (m, 7H), 2.21-2.12 (m, 2H), 1.60-1.43 (m, 5H), 1.40-1.25 (m, 7H), 1.23-1.19 (m, 3H). LCMS: (Method C) 1119.8 (M+H)⁺, Rt. 2.87 min, 99.62% (Max). HPLC: (Method C) Rt. 6.56 min, 93.80% (Max).

### Example 270. Synthesis of 3-(2-(2-(4-((R)-3-(3,6-dibromo-9H-carbazol-9-yl)-2-hydroxypropyl)piperazin-1-yl)ethoxy)ethoxy)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)propanamide (Compound 191)

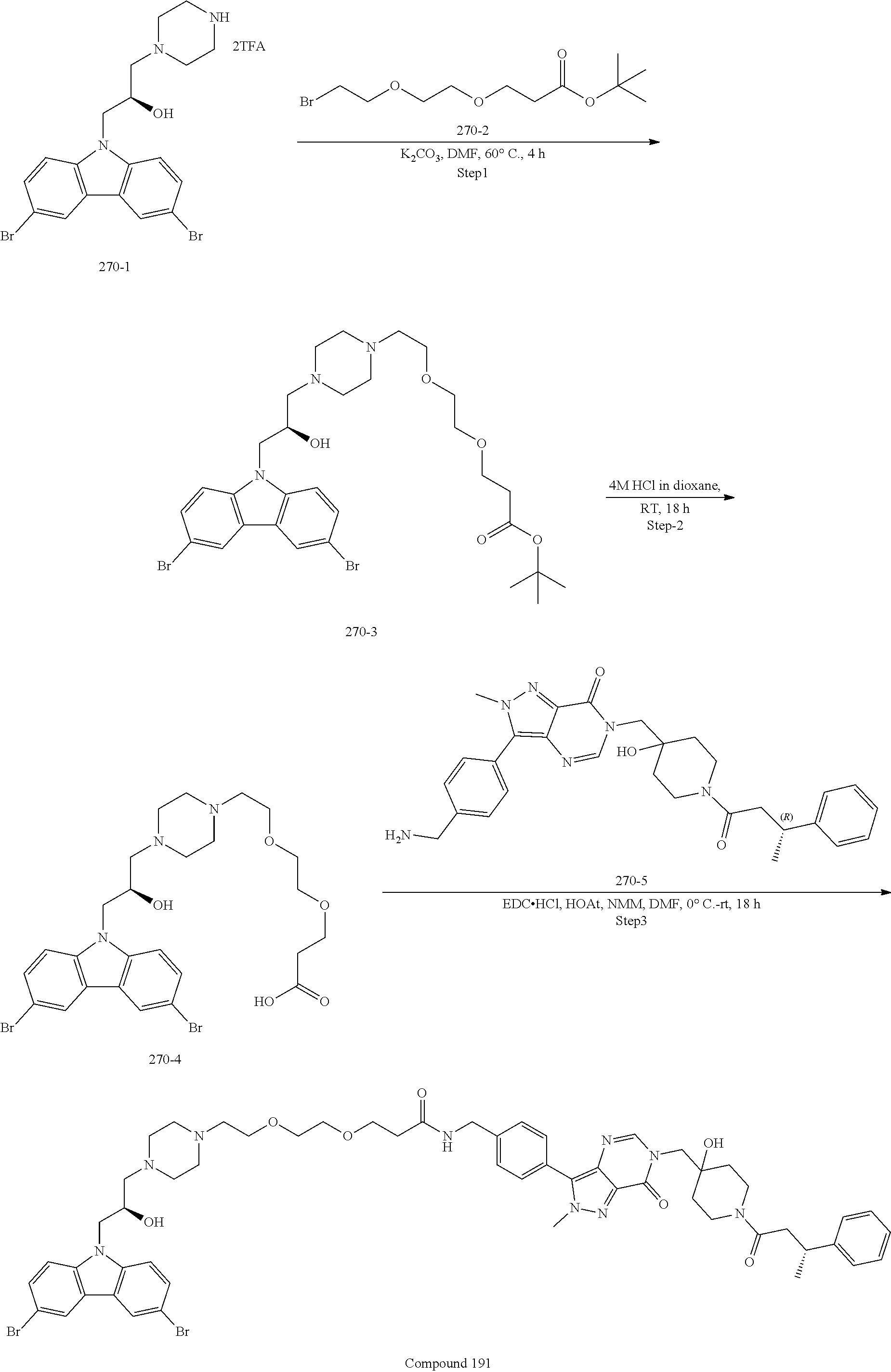

### Step 1: tert-butyl (R)-3-(2-(2-(4-(3-(3,6-dibromo-9H-carbazol-9-yl)-2-hydroxypropyl)piperazin-1-yl)ethoxy)ethoxy)propanoate (270-3)

To a solution of (R)-1-(3,6-dibromo-9H-carbazol-9-yl)-3-(piperazin-1-yl)propan-2-ol bis(2,2,2-trifluoroacetate) (270-1, 0.25 g, 0.36 mmol) in DMF (2.5 mL), K₂CO₃ (0.248 g, 1.80 mmol) and tert-butyl 3-(2-(2-bromoethoxy)ethoxy)propanoate (270-2, 0.139 g, 0.47 mmol) were added at RT and the resulting mixture was heated to 60° C. for 3.0 h. After completion (monitored by LCMS), the reaction mixture was quenched with cold-water (20 mL) and extracted with DCM (2×10 mL). The combined organic layer was dried over sodium sulphate and concentrated under reduced pressure to get the title compound (270-3, 0.17 g, 57% yield) as a pale-yellow gum. ¹H NMR (300 MHz, DMSO-d₆) δ=8.46 (d, J=1.4 Hz, 2H), 7.65-7.57 (m, 4H), 4.94 (d, J=5.0 Hz, 1H), 4.52-4.40 (m, 1H), 4.34-4.22 (m, 1H), 4.07-3.96 (m, 1H), 3.62-3.56 (m, 2H), 3.51-3.47 (m, 6H), 2.44-2.24 (m, 14H), 1.39 (s, 9H). LCMS: (Method C) 683.8 (M+H)⁺, Rt. 3.23 min, 82.77% (Max).

### Step 2: (R)-3-(2-(2-(4-(3-(3,6-dibromo-9H-carbazol-9-yl)-2-hydroxypropyl)piperazin-1-yl)ethoxy)ethoxy)propanoic acid dihydrochloride (270-4)

To a solution of tert-butyl (R)-3-(2-(2-(4-(3-(3,6-dibromo-9H-carbazol-9-yl)-2-hydroxypropyl)piperazin-1-yl)ethoxy)ethoxy)propanoate (270-3, 0.170 g, 0.25 mmol) in 1,4-dioxane (2.5 mL) was added HCl (2.49 mL, 9.95 mmol, 4 M in dioxane) at RT and the resulting mixture was stirred at RT for 18 h. After completion (monitored by LCMS), the reaction mixture was concentrated under reduced pressure. The residue was triturated with MTBE and the dried under vacuum to get the title compound (270-4, 0.17 g, 87% yield) as an off-white solid. 1H NMR (400 MHz, DMSO-d₆) δ=8.49 (d, J=1.9 Hz, 2H), 7.71 (d, J=8.6 Hz, 2H), 7.63 (dd, J=1.9, 8.8 Hz, 2H), 4.49-4.33 (m, 3H), 3.81-3.75 (m, 2H), 3.65-3.59 (m, 6H), 3.56-3.50 (m, 12H), 2.47-2.44 (m, 2H). LCMS: (Method B) 627.8 (M+H)⁺, Rt. 2.31 min, 88.85% (Max).

### Step 3: 3-(2-(2-(4-((R)-3-(3,6-dibromo-9H-carbazol-9-yl)-2-hydroxypropyl)piperazin-1-yl)ethoxy)ethoxy)-N-(4-(6-((4-hydroxy-1-((R)-3-phenylbutanoyl)piperidin-4-yl)methyl)-2-methyl-7-oxo-6,7-dihydro-2H-pyrazolo[4,3-d]pyrimidin-3-yl)benzyl)propenamide (Compound 191)

To a solution of (R)-3-(2-(2-(4-(3-(3,6-dibromo-9H-carbazol-9-yl)-2-hydroxypropyl)piperazin-1-yl)ethoxy)ethoxy)propanoic acid dihydrochloride (270-4, 0.170 g, 0.24 mmol) in DMF (3.0 mL), EDC·HCl (70 mg, 0.36 mmol), 1-hydroxy-7-azabenzotriazole (50 mg, 0.36 mmol), N-methyl morpholine (0.133 mL, 1.21 mmol) were added at 0° C. After 10 min of stirring, ((R)-3-(4-(aminomethyl)phenyl)-6-((4-hydroxy-1-(3-phenylbutanoyl)piperidin-4-yl)methyl)-2-methyl-2,6-dihydro-7H-pyrazolo[4,3-d]pyrimidin-7-one (270-5, 0.125 g, 0.24 mmol) was added and the reaction mixture was stirred for 6 h at RT. After completion (monitored by LCMS), the reaction was quenched with ice-water and extracted with DCM (2×10 mL). The combined organic layer was washed with brine (10 mL), filtered, and concentrated under reduced pressure. The crude residue was purified by reverse phase preparative HPLC (Purification method: Xbridge C18 (19×250) mm, 5 micron; Mobile phase A: 10 mM Ammonium bicarbonate and Mobile phase B: Acetonitrile, Flow rate=12 mL/minute) to get the title compound (47 mg, 17%, yield) as a white solid. ¹H NMR (400 MHz, DMSO-d₆) δ=8.49-8.42 (m, 3H), 7.97 (d, J=10.5 Hz, 1H), 7.69-7.56 (m, 6H), 7.45 (d, J=8.0 Hz, 2H), 7.30-7.22 (m, 4H), 7.19-7.12 (m, 1H), 4.97-4.91 (m, 1H), 4.87 (d, J=5.0 Hz, 1H), 4.46-4.35 (m, 3H), 4.32-4.23 (m, 1H), 4.13-4.07 (m, 3H), 4.06-3.87 (m, 4H), 3.71-3.62 (m, 3H), 3.52 (br s, 5H), 3.27-3.12 (m, 3H), 2.94-2.84 (m, 1H), 2.62-2.57 (m, 2H), 2.46-2.34 (m, 11H), 2.32-2.21 (m, 3H), 1.61-1.27 (m, 4H), 1.21 (d, J=7.0 Hz, 3H). LCMS: (Method C) 1123.9 (M+H)⁺, Rt. 2.82 min, 98.76% (Max), HPLC: (Method A) Rt. 4.28 min, 99.88 (Max) %.

All publications and patent applications cited in this specification are herein incorporated by reference as if each individual publication or patent application were specifically and individually indicated to be incorporated by referenced. All crystal structures cited by RCSB PDB code are also incorporated by reference.

Although the foregoing invention has been described in some detail by way of illustration and example for the purposes of clarity of understanding, it will be readily apparent to one of ordinary skill in the art in light of the teaching of this invention that certain changes and modifications may be made thereto without departing from the spirit or scope of the invention as defined in the embodiments and/or claims.

We claim:

1. A compound of Formula 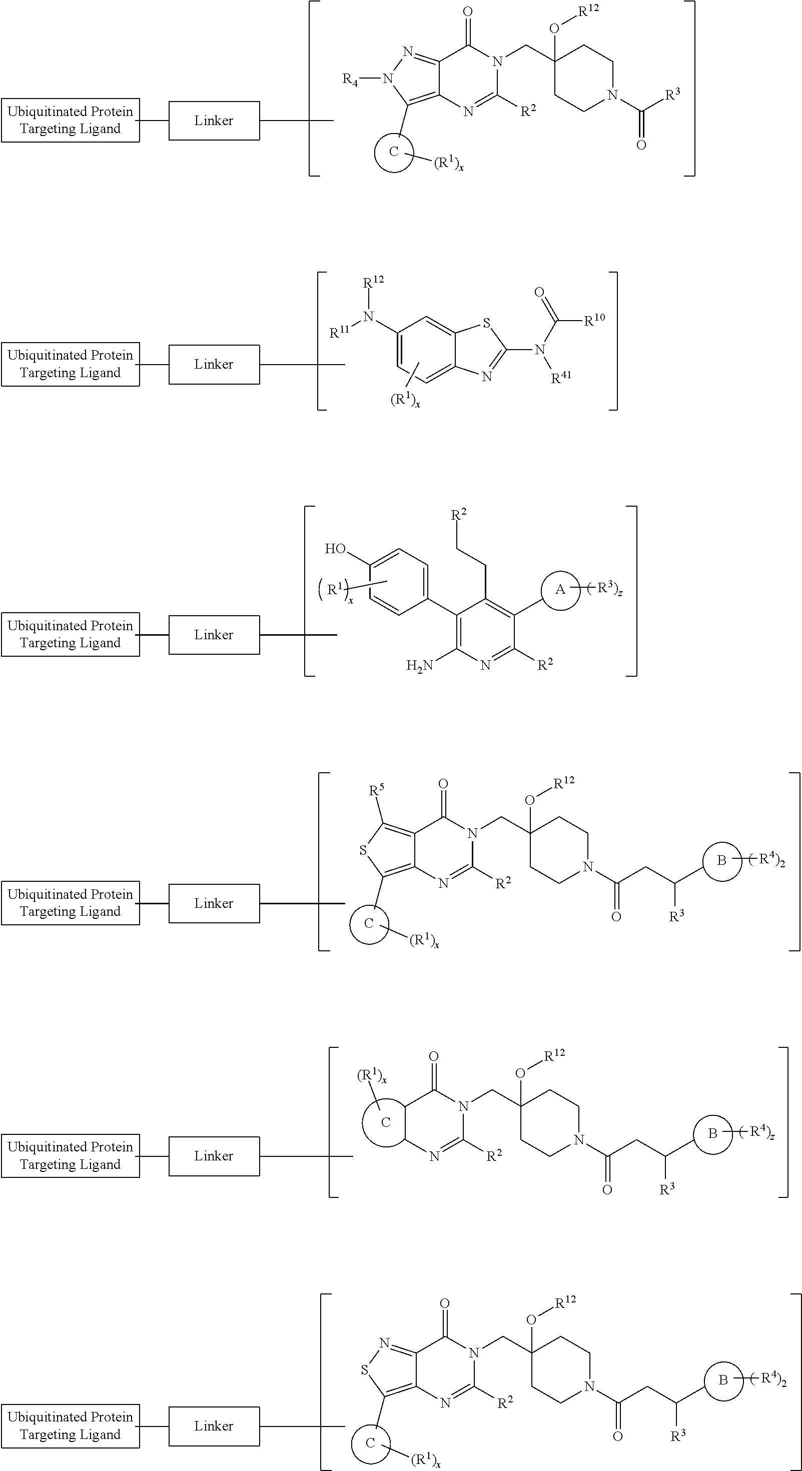 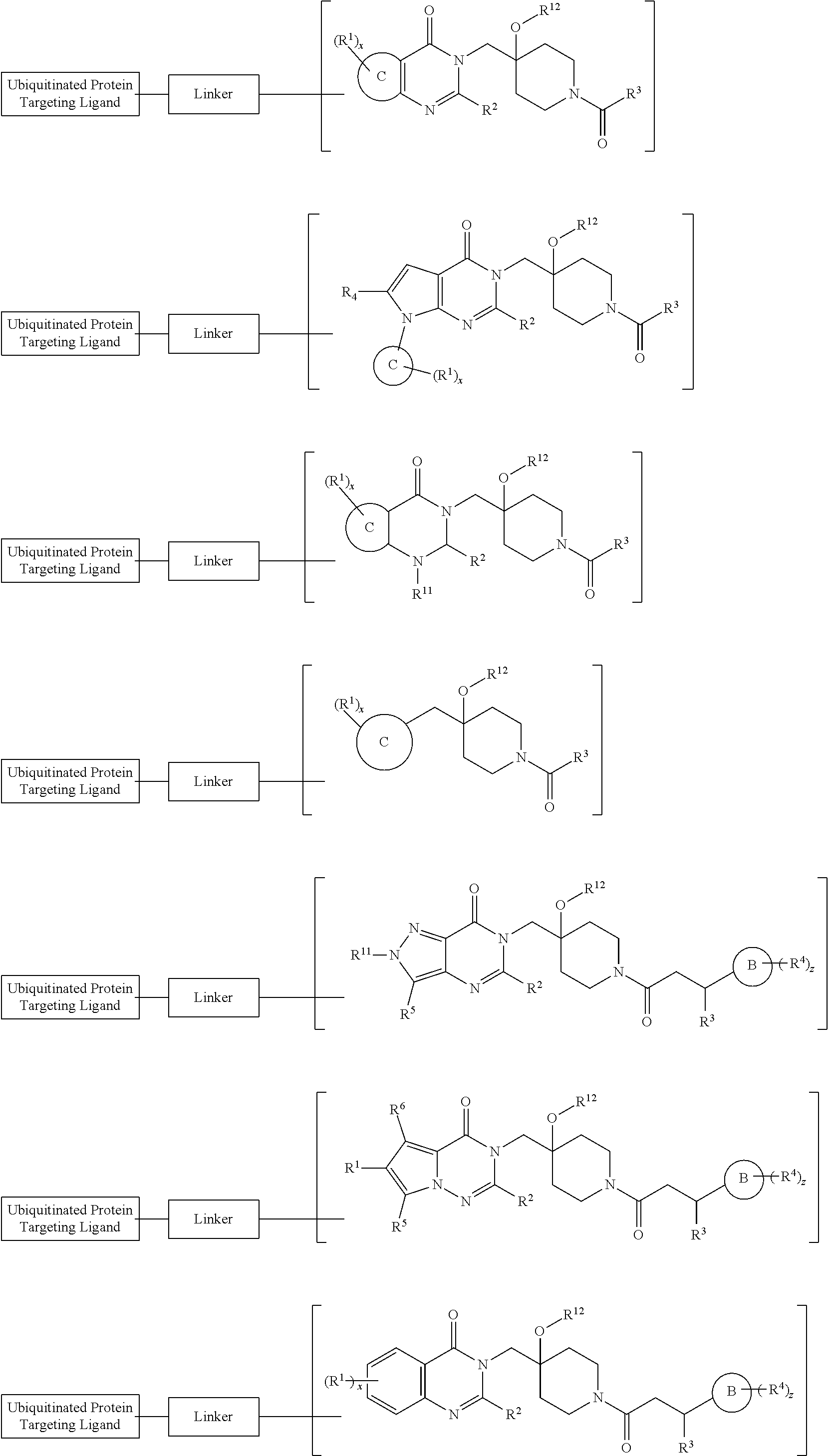 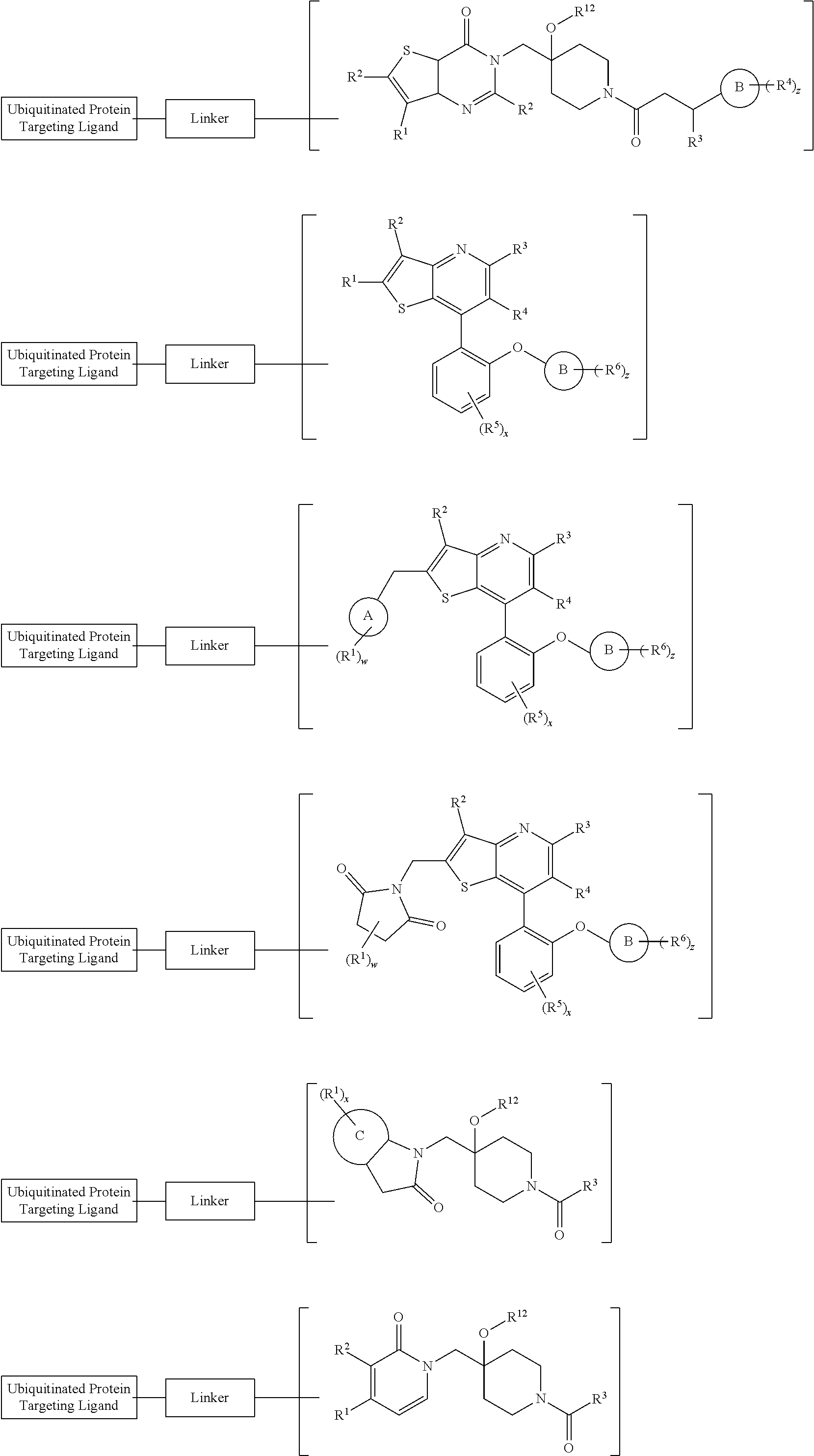 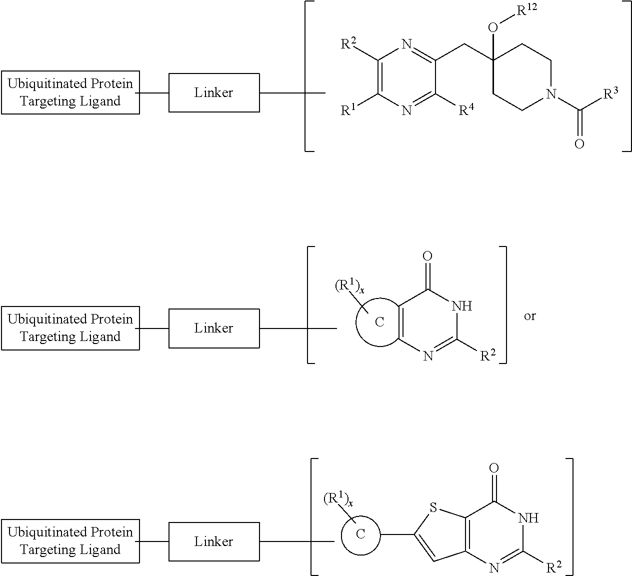
or a pharmaceutically acceptable salt thereof;
wherein: 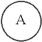
is an aryl, heteroaryl, heterocycle, or cycloalkyl group; 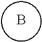
is an aryl, heteroaryl, heterocycle, or cycloalkyl group; 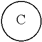
is an aryl, heteroaryl, heterocycle, or cycloalkyl group; 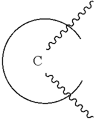
is a fused heterocycle, aryl, heteroaryl, cycloalkyl, or cycloalkenyl group;
x is 0, 1, 2, 3, or 4 as allowed by valence;
z is 0, 1, 2, 3, or 4 as allowed by valence;
w is 0, 1, 2, 3, or 4 as allowed by valence;
R¹ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²¹;
R² is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²²;
R³ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²³;
R⁴ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁴;
R⁵ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁵;
R⁶ is independently selected at each instance from hydrogen, halogen, alkyl, haloalkyl, alkenyl, alkynyl, heterocycle, aryl, heteroaryl, cyano, nitro, —C(O)R¹⁰, —OC(O)R¹⁰, —NR¹¹C(O)R¹⁰, —OR¹¹, —NR¹¹R¹², —S(O)R¹⁰, —S(O)₂R¹⁰, —OS(O)R¹⁰, —OS(O)₂R¹⁰, —NR¹¹S(O)R¹⁰, —NR¹¹S(O)₂R¹⁰, and —SR¹¹, wherein each alkyl, haloalkyl, alkenyl, alkynyl, heterocycle, aryl, and heteroaryl is optionally substituted as allowed by valence with 1, 2, 3, or 4 substituents selected from R²⁶;
R¹⁰ is independently selected at each instance from hydrogen, alkyl, haloalkyl, alkenyl, alkynyl, —OR¹¹, —NR¹¹R¹², —SR¹¹, aryl, heterocycle, and heteroaryl; each of which alkyl, haloalkyl, alkenyl, alkynyl, heterocycle, aryl, and heteroaryl is optionally substituted as allowed by valence with 1, 2, 3, or 4 substituents selected from R³⁰;
R¹¹ and R¹² are independently selected at each instance from hydrogen, alkyl, haloalkyl, alkenyl, alkynyl, aryl, heterocycle, heteroaryl, —C(O)R⁴⁰, —S(O)R⁴⁰, and —S(O)₂R⁴⁰; each of which alkyl, haloalkyl, alkenyl, alkynyl, aryl, heterocycle, and heteroaryl is optionally substituted as allowed by valence with 1, 2, 3, or 4 substituents selected from R³¹;
R²¹, R²², R²³, R²⁴, R²⁵, and R²⁶ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R³⁰ and R³¹ are independently selected at each instance from hydrogen, halogen, alkyl, haloalkyl, alkenyl, alkynyl, heterocycle, aryl, heteroaryl, cyano, nitro, —C(O)R⁴⁰, —OC(O)R⁴⁰, —NR⁴¹C(O)R⁴⁰, —OR⁴¹, —NR⁴¹R⁴², —S(O)R⁴⁰, —S(O)₂R⁴⁰, —OS(O)R⁴⁰, —OS(O)₂R⁴⁰, —NR⁴¹S(O)R⁴⁰, —NR⁴¹S(O)₂R⁴⁰, and —SR⁴¹, wherein each alkyl, haloalkyl, alkenyl, alkynyl, heterocycle, aryl, and heteroaryl is optionally substituted as allowed by valence with 1, 2, 3, or 4 substituents selected from R⁴³;
R⁴⁰ is independently selected at each instance from hydrogen, alkyl, haloalkyl, alkenyl, alkynyl, aryl, heterocycle, heteroaryl, amino, hydroxyl, alkoxy, —NHalkyl, and —N(alkyl)₂, each of which except hydrogen is optionally substituted as allowed by valence with 1, 2, 3, or 4 substituents selected from R⁴³;
R⁴¹ and R⁴² are independently selected at each instance from hydrogen, alkyl, haloalkyl, alkenyl, alkynyl, aryl, heterocycle, and heteroaryl; each of which except hydrogen is optionally substituted as allowed by valence with 1, 2, 3, or 4 substituents selected from R⁴³;
R⁴³ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl;
the Ubiquitinated Protein Targeting Ligand is a ligand that binds a Target Ubiquitinated Protein;
the Linker is a bond or a bivalent moiety that links the Protein Targeting Ligand and the USP7 Targeting; and
and wherein Linker-Ubiquitinated Protein Targeting Ligand replaces a R¹, R², R³, R⁴, R⁵, R⁶, R¹⁰, R¹¹, or R¹² group; or Linker-Ubiquitinated Protein Targeting Ligand is covalently attached to a R¹, R², R³, R⁴, R⁵, R⁶, R¹⁰, R¹¹, or R¹² group as allowed by valence; or Linker-Ubiquitinated Protein Targeting Ligand is covalently attached in a position other than R¹, R², R³, R⁴, R⁵, R⁶, R¹⁰, R¹¹, and R¹².

2. The compound of claim 1, wherein Linker-Ubiquitinated Protein Targeting Ligand replaces a R¹, R², R³, R⁴, R⁵, R⁶, R¹⁰, R¹¹, or R¹² group.

3. The compound of claim 1, wherein Linker-Ubiquitinated Protein Targeting Ligand is covalently attached to a R¹, R², R³, R⁴, R⁵, R⁶, R¹⁰, R¹¹, or R² group as allowed by valence.

4. The compound of claim 1, wherein Linker-Ubiquitinated Protein Targeting Ligand is covalently attached in a position other than R¹, R², R³, R⁴, R⁵, R⁶, R¹⁰, R¹¹, and R¹².

5. The compound of claim 1, wherein Linker-Ubiquitinated Protein Targeting Ligand replaces R1.

6. The compound of claim 1, wherein Linker-Ubiquitinated Protein Targeting Ligand replaces R².

7. The compound of claim 1, wherein Linker-Ubiquitinated Protein Targeting Ligand replaces R³.

8. The compound of claim 1, wherein Linker-Ubiquitinated Protein Targeting Ligand replaces R⁴.

9. The compound of claim 1, wherein Linker-Ubiquitinated Protein Targeting Ligand replaces R⁵.

10. The compound of claim 1, wherein Linker-Ubiquitinated Protein Targeting Ligand replaces R⁶.

11. The compound of claim 1, wherein Linker-Ubiquitinated Protein Targeting Ligand replaces R¹⁰.

12. The compound of claim 1, wherein Linker-Ubiquitinated Protein Targeting Ligand replaces R¹¹.

13. The compound of claim 1, wherein Linker-Ubiquitinated Protein Targeting Ligand replaces R¹².

14. The compound of claim 1, wherein the compound is of Formula: 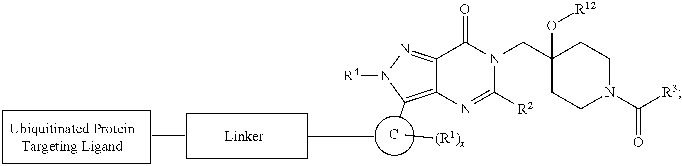
or a pharmaceutically acceptable salt thereof.

15. The compound of claim 1, wherein the compound is of Formula: 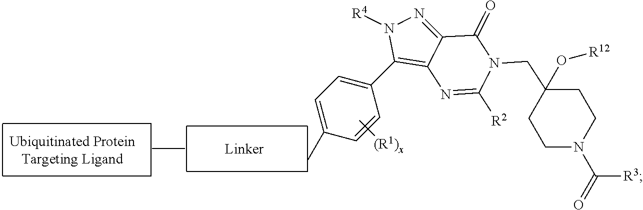
or a pharmaceutically acceptable salt thereof.

16. The compound of claim 1, wherein the compound is of Formula: 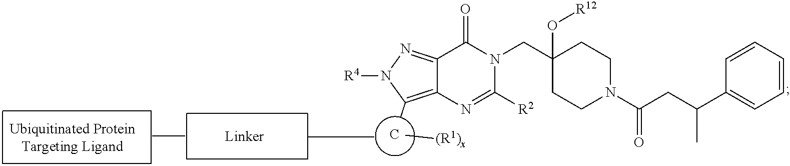
or a pharmaceutically acceptable salt thereof.

17. The compound of any one of claims 1-16, wherein R⁴ is methyl.

18. The compound of any one of claims 1-16, wherein R⁴ is hydrogen.

19. The compound of any one of claims 1-18, wherein R² is hydrogen.

20. The compound of any one of claims 1-18, wherein R² is alkyl, haloalkyl, or halogen.

21. The compound of claim 1, wherein the compound is of Formula: 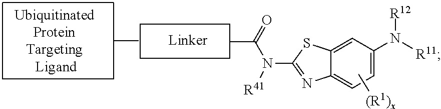
or a pharmaceutically acceptable salt thereof.

22. The compound of claim 1, wherein the compound is of Formula: 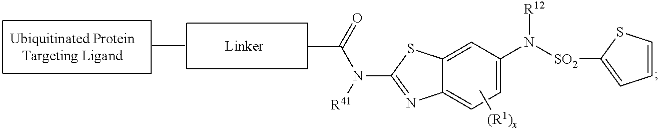
or a pharmaceutically acceptable salt thereof.

23. The compound of claim 21 or 22, wherein R⁴¹ is hydrogen.

24. The compound of claim 21 or 22, wherein R⁴¹ is alkyl.

25. The compound of any one of claims 1-24, wherein R¹² is hydrogen.

26. The compound of any one of claims 1-24, wherein R¹² is alkyl.

27. The compound of any one of claims 1-26, wherein x is 0.

28. The compound of any one of claims 1-26, wherein x is 1.

29. The compound of any one of claims 1-26, wherein x is 2.

30. The compound of any one of claims 1-26, wherein x is 3.

31. The compound of any one of claims 28-30, wherein R¹ is selected from F, Cl, alkyl, and haloalkyl.

32. The compound of any one of claims 1-31, wherein Linker is 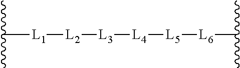
L₁, L₂, L₃, L₄, L₅, and L₆ are independently selected from the group consisting of a bond, alkyl, alkene, alkyne, haloalkyl, alkoxy, aryl, heterocycle, heteroaryl, bicycle, —C(O)—, —C(O)O—, —OC(O)—, —SO₂—, —S(O)—, —C(S)—, —C(O)NR¹¹—, —NR¹¹C(O)—, —O—, —S—, —NR¹¹—, —P(O)(OR¹¹)O—, —P(O)(OR¹¹)—, polyethylene glycol, lactic acid, and glycolic acid, each of which except bond is optionally substituted with 1, 2, 3, or 4 substituents independently selected from R⁴⁴; wherein L₁, L₂, L₃, L₄, L₅, and L₆ are selected such that there are no more than two of the same moieties connected together (e.g, L₁, L₂, and L₃ cannot all three be —C(O)—) and O and N atoms are not directly linked together except within aromatic rings (e.g. L₁ and L₂ cannot both be —O—or NR¹¹);
R⁴⁴ is independently selected at each instance from hydrogen, alkyl, haloalkyl, alkenyl, alkynyl, aryl, heterocycle, heteroaryl, amino, hydroxyl, alkoxy, —NR¹¹R¹², halogen, cyano, nitro, —OC(O)R⁴⁰, —NR¹¹C(O)R⁴⁰, —C(O)R⁴⁰, —OP(O)(R⁴⁰)₂, —P(O)(R⁴⁰)₂, —NR¹¹P(O)(R⁴⁰)₂, —SR¹¹, —OR¹¹, —S(O)R⁴⁰, —S(O)₂R⁴⁰, and —N(alkyl)C(O)R⁴⁰, each of which except hydrogen is optionally substituted as allowed by valence with 1, 2, 3, or 4 substituents selected from R⁴⁵; and
R⁴⁵ is independently selected at each instance from hydrogen, halogen, cyano, nitro, alkyl, haloalkyl, alkenyl, alkynyl, aryl, heterocycle, heteroaryl, amino, hydroxyl, alkoxy, —NHalkyl, —N(alkyl)₂, —OC(O)alkyl, —NHC(O)alkyl, and —N(alkyl)C(O)alkyl.

33. The compound of claim 31, wherein L₁ is bond.

34. The compound of claim 31, wherein L₁ is alkyl optionally substituted with 1, 2, 3, or 4 substituents independently selected from R⁴⁴.

35. The compound of claim 31, wherein L₁ is haloalkyl optionally substituted with 1, 2, 3, or 4 substituents independently selected from R⁴⁴.

36. The compound of claim 31, wherein L₁ is aryl optionally substituted with 1, 2, 3, or 4 substituents independently selected from R⁴⁴.

37. The compound of claim 31, wherein L₁ is heterocycle optionally substituted with 1, 2, 3, or 4 substituents independently selected from R⁴⁴.

38. The compound of claim 31, wherein L₁ is heteroaryl optionally substituted with 1, 2, 3, or 4 substituents independently selected from R⁴⁴.

39. The compound of claim 31, wherein L₁ is bicycle optionally substituted with 1, 2, 3, or 4 substituents independently selected from R⁴⁴.

40. The compound of claim 31, wherein L₁ is —C(O)—.

41. The compound of claim 31, wherein L₁ is —SO₂—.

42. The compound of claim 31, wherein L₁ is —C(O)O—, —OC(O)—, —NR¹¹C(O)—, and —C(O)NR¹¹—.

43. The compound of claim 31, wherein L₁ is —O—.

44. The compound of claim 31, wherein L₁ is —S—.

45. The compound of claim 31, wherein L₁ is —NR¹¹—.

46. The compound of claim 31, wherein L₁ is polyethylene glycol.

47. The compound of claim 31, wherein L₁ is lactic acid or glycolic acid.

48. The compound of any one of claims 31-47, wherein L₂ is bond.

49. The compound of any one of claims 31-47, wherein L₂ is alkyl optionally substituted with 1, 2, 3, or 4 substituents independently selected from R⁴⁴.

50. The compound of any one of claims 31-47, wherein L₂ is haloalkyl optionally substituted with 1, 2, 3, or 4 substituents independently selected from R⁴⁴.

51. The compound of any one of claims 31-47, wherein L₂ is aryl optionally substituted with 1, 2, 3, or 4 substituents independently selected from R⁴⁴.

52. The compound of any one of claims 31-47, wherein L₂ is heterocycle optionally substituted with 1, 2, 3, or 4 substituents independently selected from R⁴⁴.

53. The compound of any one of claims 31-47, wherein L₂ is heteroaryl optionally substituted with 1, 2, 3, or 4 substituents independently selected from R⁴⁴.

54. The compound of any one of claims 31-47, wherein L₂ is bicycle optionally substituted with 1, 2, 3, or 4 substituents independently selected from R⁴⁴.

55. The compound of any one of claims 31-47, wherein L₂ is polyethylene glycol.

56. The compound of any one of claims 31-47, wherein L₂ is lactic acid or glycolic acid.

57. The compound of any one of claims 31-56, wherein L₃ is bond.

58. The compound of any one of claims 31-56, wherein L₃ is alkyl optionally substituted with 1, 2, 3, or 4 substituents independently selected from R⁴⁴.

59. The compound of any one of claims 31-56, wherein L₃ is haloalkyl optionally substituted with 1, 2, 3, or 4 substituents independently selected from R⁴⁴.

60. The compound of any one of claims 31-56, wherein L₃ is aryl optionally substituted with 1, 2, 3, or 4 substituents independently selected from R⁴⁴.

61. The compound of any one of claims 31-56, wherein L₃ is heterocycle optionally substituted with 1, 2, 3, or 4 substituents independently selected from R⁴⁴.

62. The compound of any one of claims 31-56, wherein L₃ is heteroaryl optionally substituted with 1, 2, 3, or 4 substituents independently selected from R⁴⁴.

63. The compound of any one of claims 31-56, wherein L₃ is bicycle optionally substituted with 1, 2, 3, or 4 substituents independently selected from R⁴⁴.

64. The compound of any one of claims 31-56, wherein L₃ is —C(O)—.

65. The compound of any one of claims 31-56, wherein L₃ is —SO₂—.

66. The compound of any one of claims 31-56, wherein L₃ is —C(O)O—, —OC(O)—, —NR¹¹C(O)—, and —C(O)NR¹¹—.

67. The compound of any one of claims 31-56, wherein L₃ is —O—.

68. The compound of any one of claims 31-56, wherein L₃ is —S—.

69. The compound of any one of claims 31-56, wherein L₃ is —NR¹¹—.

70. The compound of any one of claims 31-56, wherein L₃ is polyethylene glycol.

71. The compound of any one of claims 31-56, wherein L₃ is lactic acid or glycolic acid.

72. The compound of any one of claims 31-71, wherein L₄ is bond.

73. The compound of any one of claims 31-71, wherein L₄ is alkyl optionally substituted with 1, 2, 3, or 4 substituents independently selected from R⁴⁴.

74. The compound of any one of claims 31-71, wherein L₄ is haloalkyl optionally substituted with 1, 2, 3, or 4 substituents independently selected from R⁴⁴.

75. The compound of any one of claims 31-71, wherein L₄ is aryl optionally substituted with 1, 2, 3, or 4 substituents independently selected from R⁴⁴.

76. The compound of any one of claims 31-71, wherein L₄ is heterocycle optionally substituted with 1, 2, 3, or 4 substituents independently selected from R⁴⁴.

77. The compound of any one of claims 31-71, wherein L₄ is heteroaryl optionally substituted with 1, 2, 3, or 4 substituents independently selected from R⁴⁴.

78. The compound of any one of claims 31-71, wherein L₄ is bicycle optionally substituted with 1, 2, 3, or 4 substituents independently selected from R⁴⁴.

79. The compound of any one of claims 31-71, wherein L₄ is polyethylene glycol.

80. The compound of any one of claims 31-71, wherein L₄ is lactic acid or glycolic acid.

81. The compound of any one of claims 31-80, wherein L₅ is bond.

82. The compound of any one of claims 31-80, wherein L₅ is alkyl optionally substituted with 1, 2, 3, or 4 substituents independently selected from R⁴⁴.

83. The compound of any one of claims 31-80, wherein L₅ is haloalkyl optionally substituted with 1, 2, 3, or 4 substituents independently selected from R⁴⁴.

84. The compound of any one of claims 31-80, wherein L₅ is aryl optionally substituted with 1, 2, 3, or 4 substituents independently selected from R⁴⁴.

85. The compound of any one of claims 31-80, wherein L₅ is heterocycle optionally substituted with 1, 2, 3, or 4 substituents independently selected from R⁴⁴.

86. The compound of any one of claims 31-80, wherein L₅ is heteroaryl optionally substituted with 1, 2, 3, or 4 substituents independently selected from R⁴⁴.

87. The compound of any one of claims 31-80, wherein L₅ is bicycle optionally substituted with 1, 2, 3, or 4 substituents independently selected from R⁴⁴.

88. The compound of any one of claims 31-80, wherein L₅ is —C(O)—.

89. The compound of any one of claims 31-80, wherein L₅ is —SO₂—.

90. The compound of any one of claims 31-80, wherein L₅ is —C(O)O—, —OC(O)—, —NR¹¹C(O)—, and —C(O)NR¹¹—.

91. The compound of any one of claims 31-80, wherein L₅ is —O—.

92. The compound of any one of claims 31-80, wherein L₅ is —S—.

93. The compound of any one of claims 31-80, wherein L₅ is —NR¹¹—.

94. The compound of any one of claims 31-80, wherein L₅ is polyethylene glycol.

95. The compound of any one of claims 31-80, wherein L₅ is lactic acid or glycolic acid.

96. The compound of any one of claims 31-95, wherein L₆ is bond.

97. The compound of any one of claims 31-95, wherein L₆ is alkyl optionally substituted with 1, 2, 3, or 4 substituents independently selected from R⁴⁴.

98. The compound of any one of claims 31-95, wherein L₆ is haloalkyl optionally substituted with 1, 2, 3, or 4 substituents independently selected from R⁴⁴.

99. The compound of any one of claims 31-95, wherein L₆ is aryl optionally substituted with 1, 2, 3, or 4 substituents independently selected from R⁴⁴.

100. The compound of any one of claims 31-95, wherein L₆ is heterocycle optionally substituted with 1, 2, 3, or 4 substituents independently selected from R⁴⁴.

101. The compound of any one of claims 31-95, wherein L₆ is heteroaryl optionally substituted with 1, 2, 3, or 4 substituents independently selected from R⁴⁴.

102. The compound of any one of claims 31-95, wherein L₆ is bicycle optionally substituted with 1, 2, 3, or 4 substituents independently selected from R⁴⁴.

103. The compound of any one of claims 31-95, wherein L₆ is polyethylene glycol.

104. The compound of any one of claims 31-95, wherein L₆ is lactic acid or glycolic acid.

105. The compound of any one of claims 31-95, wherein L₁ is bound to USP7 Targeting Ligand.

106. The compound of any one of claims 31-95, wherein L₁ is bound to Ubiquitinated Protein Targeting Ligand.

107. The compound of any one of claims 31-106, wherein R⁴⁴ is independently selected at each instance from alkyl, halogen, and haloalkyl.

108. The compound of any one of claims 31-106, wherein R⁴⁴ is alkyl optionally substituted as allowed by valence with 1, 2, 3, or 4 substituents selected from R⁴⁵.

109. The compound of any one of claims 31-106, wherein R⁴⁴ is aryl optionally substituted as allowed by valence with 1, 2, 3, or 4 substituents selected from R⁴⁵.

110. The compound of any one of claims 31-106, wherein R⁴⁴ is heterocycle optionally substituted as allowed by valence with 1, 2, 3, or 4 substituents selected from R⁴⁵.

111. The compound of any one of claims 31-106, wherein R⁴⁴ is heteroaryl optionally substituted as allowed by valence with 1, 2, 3, or 4 substituents selected from R⁴⁵.

112. The compound of any one of claims 31-106, wherein R⁴⁴ is amino optionally substituted as allowed by valence with 1, 2, 3, or 4 substituents selected from R⁴⁵.

113. The compound of any one of claims 31-106, wherein R⁴⁴ is hydroxyl optionally substituted as allowed by valence with 1, 2, 3, or 4 substituents selected from R⁴⁵.

114. The compound of any one of claims 31-106, wherein R⁴⁴ is alkoxy optionally substituted as allowed by valence with 1, 2, 3, or 4 substituents selected from R⁴⁵.

115. The compound of any one of claims 31-114, wherein R⁴⁵ is independently selected from halogen, alkyl, and haloalkyl.

116. The compound of any one of claims 31-114, wherein R⁴⁵ is independently selected from amino, hydroxyl, alkoxy, —NHalkyl, —N(alkyl)₂, —OC(O)alkyl, —NHC(O)alkyl, and —N(alkyl)C(O)alkyl.

117. The compound of any one of claims 1-116, wherein the Ubiquitinated Protein Targeting Ligand is a ligand that binds CFTR.

118. The compound of claim 117, wherein the Ubiquitinated Protein Targeting Ligand is selected from FIG. 2A, FIG. 2B, FIG. 2C, and FIG. 2D.

119. The compound of any one of claims 1-116, wherein the Ubiquitinated Protein Targeting Ligand is a ligand that binds phenylalanine hydroxylase.

120. The compound of claim 119, wherein the Ubiquitinated Protein Targeting Ligand is selected from FIG. 3A, FIG. 3B, and FIG. 3C.

121. The compound of any one of claims 1-116, wherein the Ubiquitinated Protein Targeting Ligand is a ligand that binds p53.

122. The compound of claim 121, wherein the Ubiquitinated Protein Targeting Ligand is selected from FIG. 4A, FIG. 4B, and FIG. 4C.

123. The compound of any one of claims 1-116, wherein the Ubiquitinated Protein Targeting Ligand is a ligand that binds rhodopsin.

124. The compound of claim 123, wherein the Ubiquitinated Protein Targeting Ligand is selected from FIG. 5A and FIG. 5B.

125. The compound of any one of claims 1-116, wherein the Ubiquitinated Protein Targeting Ligand is a ligand that binds c-myc.

126. The compound of claim 125, wherein the Ubiquitinated Protein Targeting Ligand is selected from FIG. 6A and FIG. 6B.

127. The compound of any one of claims 1-116, wherein the Ubiquitinated Protein Targeting Ligand is a ligand that binds RIPK1.

128. The compound of claim 127, wherein the Ubiquitinated Protein Targeting Ligand is selected from FIG. 7A, FIG. 7B, FIG. 7C, FIG. 7D, and FIG. 7E.

129. The compound of any one of claims 1-116, wherein the Ubiquitinated Protein Targeting Ligand is a ligand that binds RIPK1.

130. The compound of claim 129, wherein the Ubiquitinated Protein Targeting Ligand is selected from FIG. 8.

131. The compound of any one of claims 1-116, wherein the Ubiquitinated Protein Targeting Ligand is a ligand that binds CDKN1B.

132. The compound of claim 131, wherein the Ubiquitinated Protein Targeting Ligand is selected from FIG. 9A and FIG. 9B.

133. The compound of any one of claims 1-116, wherein the Ubiquitinated Protein Targeting Ligand is a ligand that binds ABCA4.

134. The compound of claim 133, wherein the Ubiquitinated Protein Targeting Ligand is selected from FIG. 10.

135. The compound of any one of claims 1-116, wherein the Ubiquitinated Protein Targeting Ligand is a ligand that binds ABCB11.

136. The compound of claim 136, wherein the Ubiquitinated Protein Targeting Ligand is selected from FIG. 11A and FIG. 11B.

137. The compound of any one of claims 1-116, wherein the Ubiquitinated Protein Targeting Ligand is a ligand that binds choline acetylase.

138. The compound of claim 137, wherein the Ubiquitinated Protein Targeting Ligand is selected from FIG. 12.

139. The compound of any one of claims 1-116, wherein the Ubiquitinated Protein Targeting Ligand is a ligand that binds CYLD.

140. The compound of claim 139, wherein the Ubiquitinated Protein Targeting Ligand is selected from FIG. 13.

141. The compound of any one of claims 1-116, wherein the Ubiquitinated Protein Targeting Ligand is a ligand that binds NEMO.

142. The compound of claim 141, wherein the Ubiquitinated Protein Targeting Ligand is selected from FIG. 14.

143. The compound of any one of claims 1-116, wherein the Ubiquitinated Protein Targeting Ligand is a ligand that binds AH receptor-interacting protein.

144. The compound of claim 143, wherein the Ubiquitinated Protein Targeting Ligand is selected from FIG. 15A and FIG. 15B.

145. The compound of any one of claims 1-116, wherein the Ubiquitinated Protein Targeting Ligand is a ligand that binds PDCD4.

146. The compound of claim 145, wherein the Ubiquitinated Protein Targeting Ligand is selected from FIG. 16.

147. The compound of any one of claims 1-116, wherein the Ubiquitinated Protein Targeting Ligand is a ligand that binds RIPK2.

148. The compound of claim 147, wherein the Ubiquitinated Protein Targeting Ligand is selected from FIG. 17A, FIG. 17B, FIG. 17C, and FIG. 17D.

149. The compound of any one of claims 1-116, wherein the Ubiquitinated Protein Targeting Ligand is a ligand that binds BAX.

150. The compound of claim 149, wherein the Ubiquitinated Protein Targeting Ligand is selected from FIG. 18A, FIG. 18B, and FIG. 18C.

151. The compound of any one of claims 1-116, wherein the Ubiquitinated Protein Targeting Ligand is a ligand that binds P21.

152. The compound of claim 151, wherein the Ubiquitinated Protein Targeting Ligand is selected from FIG. 19A and FIG. 19B.

153. The compound of any one of claims 1-116, wherein the Ubiquitinated Protein Targeting Ligand is a ligand that binds SERPINA1.

154. The compound of claim 153, wherein the Ubiquitinated Protein Targeting Ligand is selected from FIG. 20.

155. The compound of any one of claims 1-116, wherein the Ubiquitinated Protein Targeting Ligand is a ligand that binds PKLR.

156. The compound of claim 155, wherein the Ubiquitinated Protein Targeting Ligand is selected from FIG. 21A, FIG. 21B, and FIG. 21C.

157. The compound of any one of claims 1-116, wherein the Ubiquitinated Protein Targeting Ligand is a ligand that binds KEAP1.

158. The compound of claim 157, wherein the Ubiquitinated Protein Targeting Ligand is selected from FIG. 22.

159. The compound of any one of claims 1-116, wherein the Ubiquitinated Protein Targeting Ligand is a ligand that binds PTEN.

160. The compound of claim 159, wherein the Ubiquitinated Protein Targeting Ligand is selected from FIG. 23.

161. The compound of any one of claims 1-116, wherein the Ubiquitinated Protein Targeting Ligand is a ligand that binds IRAK4.

162. The compound of claim 161, wherein the Ubiquitinated Protein Targeting Ligand is selected from FIG. 24.

163. The compound of any one of claims 1-116, wherein the Ubiquitinated Protein Targeting Ligand is a ligand that binds TK2.

164. The compound of claim 163, wherein the Ubiquitinated Protein Targeting Ligand is selected from FIG. 25A and FIG. 25B.

165. The compound of any one of claims 1-116, wherein the Ubiquitinated Protein Targeting Ligand is a ligand that binds KCNQ1.

166. The compound of claim 165, wherein the Ubiquitinated Protein Targeting Ligand is selected from FIG. 26.

167. The compound of any one of claims 1-116, wherein the Ubiquitinated Protein Targeting Ligand is a ligand that binds STING1.

168. The compound of claim 167, wherein the Ubiquitinated Protein Targeting Ligand is selected from FIG. 27.

169. A pharmaceutical composition comprising an effective amount of a compound of any one of claims 1-168 or a pharmaceutically acceptable salt thereof and a pharmaceutically acceptable carrier.

170. A method of treating a disorder mediated by the Target Ubiquitinated Protein in a human comprising administering an effective amount of a compound or a pharmaceutically acceptable salt thereof of any one of claims 1-168.

171. A compound of any one of claims 1-168 or a pharmaceutically acceptable salt thereof for use in the manufacture of a medicament for the treatment of a disorder mediated by the Target Ubiquitinated Protein.

172. Use of a compound of any one of claims 1-168 or a pharmaceutically acceptable salt thereof in the treatment of a disorder mediated by the Target Ubiquitinated Protein in a human.

173. A pharmaceutical composition that comprises an effective amount of a compound of any one of claims 1-168 or a pharmaceutically acceptable salt thereof for use in the treatment of a disorder mediated by the Target Ubiquitinated Protein in a human optionally with a pharmaceutically acceptable carrier.
